(19) United States
(12) Patent Application Publication
(10) Pub. No.: US 2005/0233341 A1
(43) Pub. Date: Oct. 20, 2005

(54) Methods for identifying risk of melanoma and treatments thereof

(76) Inventors: Richard R. Roth, La Jolla, CA (US); Matthew Roberts Nelson, San Marco, CA (US); Stefan M. Kammerer, San Diego, CA (US); Andreas Braun, San Diego, CA (US); Carolyn R. Hoyal-Wrightson, San Diego, CA (US)

Correspondence Address: BIOTECHNOLOGY LAW GROUP, c/o PORTFOLIO IP, P.O. BOX 52050, MINNEAPOLIS, MN, 55402, US

(21) Appl. No.: 10/840,590

(22) Filed: May 5, 2004

Related U.S. Application Data
(60) Provisional application No. 60/489,703, filed on Jul. 23, 2003.

(51) Int. Cl. C12Q 1/68; A61K 38/16; A61K 31/4745; A61K 33/24

(52) U.S. Cl. 435/6; 424/649; 514/8; 514/283

(57) ABSTRACT

Provided herein are methods for identifying risk of melanoma in a subject and/or subjects at risk of melanoma, reagents and kits for carrying out the methods, methods for identifying candidate therapeutics for treating melanoma, therapeutic methods for treating melanoma in a subject and compositions comprising one or more melanoma cells and one or more NRP1, NID2, ENDO180, CDK10, FPGT, CARK, PCLO or REPS2 directed agents. These embodiments are based upon an analysis of polymorphic variations in a NRP1, NID2, ENDO180, CDK10, FPGT, CARK, PCLO or REPS2 nucleic acid, exemplified by nucleotide sequences of SEQ ID NO: 1-17.

### RELATED PATENT APPLICATIONS

This patent application claims the benefit of provisional patent application 60/489,703 filed Jul. 23, 2003, having attorney docket number 524593004001. This patent application also claims the benefit of U.S. patent application Ser. Nos. 10/703,817, 10/704,513 and 10/703,789, each filed on Nov. 6, 2003, and having attorney docket no. 524592006100, 524592006200 and 524592006300, respectively. Each of the above-identified patent applications names Richard B. Roth et al. as inventors and is hereby incorporated herein by reference in its entirety, including all drawings and cited documents.

### FIELD OF THE INVENTION

The invention relates to genetic methods for identifying risk of melanoma and treatments that specifically target the disease.

### BACKGROUND

In some parts of the world, especially among western countries, the number of people who develop melanoma is increasing faster than any other cancer. In the United States, for example, the number of new cases of melanoma has more than doubled in the past twenty years. The probability of developing melanoma increases with age, but this disease effects people of all age groups. Melanoma is one of the most common cancers in young adults.

Melanoma occurs when melanocytes (pigment cells) become malignant. Most pigment cells are in skin, and when melanoma begins its etiology in the skin it is referred to as coetaneous melanoma. Melanoma may also occur in the eye and is called ocular melanoma or intraocular melanoma. Rarely, melanoma arises in the meninges, the digestive tract, lymph nodes or other areas where melanocytes are found. Within the skin, melanocytes are located throughout the lower part of the epidermis, the latter being the surface layer of the skin. Melanocytes produce melanin, which is the pigment that gives skin its natural color. When skin is exposed to the sun, melanocytes produce more pigment, causing the skin to tan or darken.

Sometimes, clusters of melanocytes and surrounding tissue form benign growths referred to as moles or nevi (singular form is nevus). Cells in or near the nevi can divide without control or order and form malignant tumors. When melanoma spreads, cancer cells often are found in the lymph nodes. If the cancer has reached the lymph nodes, it may mean that cancer cells have spread to other parts of the body such as the liver, lungs or brain, giving rise to metastatic melanoma.

Melanoma is currently diagnosed by assessing risk factors and by performing biopsies. Risk factors for melanoma are a family history of melanoma, the presence of dysplastic nevi, patient history of melanoma, weakened immune system, many ordinary nevi, exposure levels to ultraviolet radiation, exposure to severe sunburns especially as a child or teenager, and fair skin. In a biopsy, a pathologist typically examines the biopsied tissue under a microscope to identify cancer cells. Depending upon the thickness of a tumor, if one exists, a physician may order chest x-ray, blood tests, liver scans, bone scans, and brain scans to determine whether the cancer spread to other tissues. Also, a test that identifies p16 nucleotide sequences is sold.

Upon a diagnosis of melanoma, the standard treatment is surgery. Side effects of surgery typically are pain and scarring. Surgery is generally not effective, however, in controlling melanoma that is known to have spread to other parts of the body. In such cases, physicians may utilize other methods of treatment, such as chemotherapy, biological therapy, radiation therapy, or a combination of these methods. Chemotherapy agents for treating melanoma include cisplatin, vinblastine, and dacarbazine. Chemotherapy can lead to side effects such as an increased probability of infection, bruising and bleeding, weakness and fatigue, hair loss, poor appetite, nausea and vomiting, and mouth and lip sores. Side effects of radiation therapy include fatigue and hair loss in the treated area. Biological therapies currently utilized for treatment of melanoma include interferon and interleuken-2. Side effects caused by biological therapies include flu-like symptoms, such as chills, fever, muscle aches, weakness, loss of appetite, nausea, vomiting, and diarrhea; bleeding and bruising skin; rashes, and swelling.

Certain melanoma therapeutics are in clinical trials. For example, canvaxin, which is a whole cell allogenic vaccine developed by irradiating tumor cells from two different patients, is under study. In addition, MAGE-1 and 3 minigenes and peptides and gp 100 peptides are being tested. Upcoming studies include testing of agents such as dacarbazine with a bcl-2 antisense oligonucleotide, and paclitaxel in combination with a matrix metalloprotease inhibitor.

### SUMMARY

It has been discovered that polymorphic variations in NRP1, NID2, ENDO180, CDK10, FPGT, CARK, PCLO and/or REPS2 loci in human genomic DNA are associated with occurrence of melanoma. Thus, featured herein are methods for identifying a subject at risk of melanoma and/or determining risk of melanoma in a subject, which comprise detecting the presence or absence of one or more polymorphic variations associated with melanoma in a nucleic acid sample from the subject. The one or more polymorphic variations often are detected in or near a NRP1, NID2, ENDO180, CDK10, FPGT, CARK, PCLO or REPS2 nucleotide sequence, which are set forth as SEQ ID NO: 1-17 respectively, or a substantially identical nucleotide sequence thereof.

Also featured are nucleic acids that encode a NRP1, NID2 ENDO180, CDK10, FPGT, CARK, PCLO or REPS2 polypeptide, and include one or more polymorphic variations associated with melanoma, and oligonucleotides which hybridize to those nucleic acids. Also provided are polypeptides encoded by nucleic acids having a NRP1, NID2, ENDO180, CDK10, FPGT, CARK, PCLO or REPS2 nucleotide sequence, which include the full-length polypeptide, isoforms and fragments thereof. In an embodiment, an NID2 protein is encoded which includes a serine at amino acid position 656. In addition, featured are methods for genotyping a nucleic acid from an individual at positions described herein, methods for identifying candidate therapeutic molecules for treating melanoma and related disorders, as well as methods of treating melanoma in a subject by administering a therapeutic molecule.

Also provided are compositions comprising a melanoma cell and/or a NRP1, NID2, ENDO180, CDK10, FPGT, CARK, PCLO or REPS2 nucleic acid, or a fragment or substantially identical nucleic acid thereof, with a RNAi, siRNA, a complementary or antisense DNA or RNA, or ribozyme nucleic acid designed from a NRP1, NID2, ENDO180, CDK10, FPGT, CARK, PCLO or REPS2 nucleotide sequence. In an embodiment, the nucleic acid is designed from a NRP1, NID2, ENDO180, CDK10, FPGT, CARK, PCLO or REPS2 nucleotide sequence that includes one or more melanoma associated polymorphic variations, and in some instances, specifically interacts with such a nucleotide sequence. Further, provided are arrays of nucleic acids bound to a solid surface, in which one or more nucleic acid molecules of the array are NRP1, NID2, ENDO180, CDK10, FPGT, CARK, PCLO or REPS2 nucleic acids, or a fragment or substantially identical nucleic acid thereof, or a complementary nucleic acid of the foregoing. Featured also are compositions comprising a melanoma cell and/or a protein, polypeptide or peptide encoded by a NRP1, NID2, ENDO180, CDK10, FPGT, CARK, PCLO or REPS2 nucleic acid with an antibody that specifically binds to the protein, polypeptide or peptide. In an embodiment, the antibody specifically binds to an epitope in a NRP1, NID2, ENDO180, CDK10, FPGT, CARK, PCLO or REPS2 protein, polypeptide or peptide that includes a non-synonymous amino acid modification associated with melanoma, such as a serine at position 656 of a NID2 protein, polypeptide or peptide.

### BRIEF DESCRIPTION OF THE DRAWINGS

FIGS. 1A, 1B and 1C show proximal SNPs in and around a NRP1 region for males and females combined, females alone, and males alone, respectively. FIGS. 2A, 2B and 2C show proximal SNPs in and around a ENDO180 region for males and females combined, females alone, and males alone, respectively. FIGS. 3A, 3B and 3C show proximal SNPs in and around a CDK10 region for males and females combined, females alone, and males alone, respectively. FIGS. 4A, 4B and 4C show proximal SNPs in and around a PCLO region for males and females combined, females alone, and males alone, respectively. FIGS. 5A, 5B and 5C show proximal SNPs in and around a FPGT/CARK region for males and females combined, females alone, and males alone, respectively. FIGS. 6A, 6B and 6C show proximal SNPs in and around a REPS2 region for males and females combined, females alone, and males alone, respectively. The position of each SNP on the chromosome is shown on the x-axis and the y-axis provides the negative logarithm of the p-value comparing the estimated allele to that of the control group. Also shown are exons and introns of the genes in the approximate chromosomal positions.

FIG. 7A depicts effects of NRP1-directed siRNA on melanoma M14 cell line proliferation according to a WST 1 assay. FIG. 7B shows effects of NRP1-directed siRNA on melanoma A375 cell line proliferation according to a WST 1 assay.

FIG. 8 depicts effects of CDK10_siRNA on melanoma A375 cell line proliferation according to a WST-1 assay.

### DETAILED DESCRIPTION

It has been discovered that polymorphic variants in and around NRP1, NID2, ENDO180, CDK10, FPGT, CARK, PCLO or REPS2 nucleotide sequences are associated with occurrence of melanoma in subjects. Thus, detecting genetic determinants associated with an increased risk of melanoma occurrence can lead to early identification of melanoma or susceptibility to melanoma and early prescription of preventative measures and treatments. Also, associating NRP1, NID2, ENDO180, CDK10, FPGT, CARK, PCLO or REPS2 polymorphic variants with melanoma has provided new targets for screening molecules useful in melanoma prognostics/diagnostics and melanoma treatments.

Melanoma and Sample Selection

Melanoma is typically described as a malignant neoplasm derived from cells capable of forming melanin. Melanomas arise most commonly in the skin of any part of the body, or in the eye, and rarely, in the mucous membranes of the genitalia, anus, oral cavity, or other sites. Melanoma occurs mostly in adults and may originate de novo or from a pigmented nevus or lentigo maligna. Melanomas frequently metastasize widely to regions such as lymph-nodes, skin, liver, lungs, and brain.

In the early phases, the cutaneous form is characterized by proliferation of cells at the dermal-epidermal junction that soon invade adjacent tissues. The cells vary in amount and pigmentation of cytoplasm; the nuclei are relatively large and irregular in shape, with prominent acidophilic nucleoli; and mitotic figures tend to be numerous. Other criteria for melanomas are asymmetry, irregular borders, heterogeneous color, large diameter, and a recent change in shape, size or pigmentation. Excised melanoma skin samples are often subjected to the following analyses: diagnosis of the melanocytic nature of the lesion and confirmation of its malignancy; maximum tumor thickness in millimeters (according to Breslow's method); assessment of completeness of excision of invasive and in situ components and microscopic measurements of the shortest extent of clearance; level of invasion (Clark); presence and extent of regression; presence and extent of ulceration; histological type and special variants; pre-existing lesion; mitotic rate; vascular invasion; neurotropism; cell type; tumor lymphocyte infiltration; and growth phase, vertical or radial.

Based in part upon selection criteria set forth above, individuals having melanoma can be selected for genetic studies. Also, individuals having no history of cancer or melanoma often are selected for genetic studies. Other selection criteria can include: a tissue or fluid sample is derived from an individual characterized as Caucasian; a sample is derived from an individual of German paternal and maternal descent; and relevant phenotype information is available for the individual. Phenotype information corresponding to each individual can include sex of the individual, number of nevi (e.g., actual number or relative number (e.g., few, moderate, numerous)), hair color (e.g., black, brown, blond, red), diagnosis of melanoma (e.g., tumor thickness, date of primary diagnosis, age of individual as of primary diagnosis, post-operative tumor classification, presence of nodes, occurrence of metastases, subtype, location), country or origin of mother and father, presence of certain conditions for each individual (e.g., coronary heart disease, cardiomyopathy, arteriosclerosis, abnormal blood clotting/thrombosis, emphysema, asthma, diabetes type 1, diabetes type 2, Alzheimer's disease, epilepsy, schizophrenia, manic depression/bipolar disorder, autoimmune disease, thyroid disorder, and hypertension), presence of cancer in the donor individual or blood relative (e.g., melanoma, basaliom/spinaliom/lentigo malignant/mycosis fungoides, breast cancer, colon cancer, rectum cancer, lung cancer, lung cancer, bronchus cancer, prostate cancer, stomach cancer, leukemia, lymphoma, or other cancer in donor, donor parent, donor aunt or uncle, donor offspring or donor grandparent).

Provided herein is a set of blood samples and a set of corresponding nucleic acid samples isolated from the blood samples, where the blood samples are donated from individuals diagnosed with melanoma. The sample set often includes blood samples or nucleic acid samples from 100 or more, 150 or more, or 200 or more individuals having melanoma, and sometimes from 250 or more, 300 or more, 400 or more, or 500 or more individuals. The individuals can have parents from any place of origin, and in an embodiment, the set of samples are extracted from individuals of German paternal and German maternal ancestry. The samples in each set may be selected based upon five or more criteria and/or phenotypes set forth above.

Polymorphic Variants Associated with Melanoma

A genetic analysis described hereafter linked melanoma with polymorphic variants of NRP1, NID2, ENDO180, CDK10, FPGT, CARK, PCLO or REPS2 from human subjects. Genomic nucleotide sequences representative of NRP1, NID2, ENDO180, CDK10, FPGT, CARK, PCLO or REPS2 nucleic acids are set forth in SEQ ID NOS: 1-7, respectively, and are incorporated herein by reference from published database entries (see Examples section and http address at www.ncbi.nlm.nih.gov/LocusLink/). The following is a description of molecules encoded by such regions.

NRP1

NRP1 is known as neuropilin 1, NRP, VEGF165R (Vascular Endothelial Growth Factor-165 Receptor). NRP1 protein is 923 amino acids in length and the corresponding genomic nucleotide sequence has been mapped to chromosomal position 10p 12.

Neuropilin is a type I transmembrane protein and is an epitope recognized by a monoclonal antibody that labels specific subsets of axons in the developing Xenopus nervous system. Neuropilin comprises in its extracellular domain several distinctive motifs; its cytoplasmic domain is short (40 amino acids) and is highly conserved among Xenopus, mouse, and chick. An aberrant retinal pathway and visual centers in Xenopus tadpoles share a common cell surface molecule, A5 antigen (Dev. Biol. 135: 231-240, 1989). The gene encoding human neuropilin was cloned and the structure of the protein product was characterized (He & Tessier-Lavigne, Neuropilin is a receptor for the axonal chemorepellent semaphorin III. Cell 90: 739-751, 1997).

A VEGF receptor (NCBI MIM 192240) was discovered which reportedly binds VEGF165 but not VEGF121. This isoform-specific VEGF receptor (VEGF165R) is identical to human neuropilin-1, which is also a receptor for the collapsin/semaphorin family that mediates neuronal cell guidance (Soker et al., Neuropilin-1 is expressed by endothelial and tumor cells as an isoform-specific receptor for vascular endothelial growth factor. Cell 92: 735-745, 1998). It also was shown that when coexpressed in cells with KDR (VEGFR2; NCBI MIM 191306), neuropilin-1 enhances the binding of VEGF165 to KDR and VEGF165-mediated chemotaxis. Conversely, inhibition of VEGF165 binding to neuropilin-1 inhibits its binding to KDR and its mitogenic activity for endothelial cells. It was proposed that neuropilin-1 is a novel VEGF receptor that modulates VEGF binding to KDR and subsequent bioactivity and therefore may regulate VEGF-induced angiogenesis.

It was concluded that NRP1-mediated interactions are a necessary element in the initiation of the primary immune response and offer another example, like that of agrin (NCBI MIM 103320), of a molecule shared by neurologic and immunologic synapses (Tordjman et al., A neuronal receptor, neuropilin-1, is essential for the initiation of the primary immune response. Nature Immun. 3: 477-482, 2002). It was shown that VEGF selectively upregulates NRP1 but not NRP2 via the VEGF receptor 2-dependent pathway. In a murine model of VEGF-dependent angioproliferative retinopathy, intense NRP1 mRNA expression was observed in the newly formed vessels. Furthermore, selective NRP1 inhibition in this model suppressed neovascular formation substantially. These results suggested that VEGF can not only activate endothelial cells directly but also contribute to robust angiogenesis in vivo by a mechanism that involves upregulation of its cognate receptor expression (Oh et al., Selective induction of neuropilin-1 by vascular endothelial growth factor (VEGF): a mechanism contributing to VEGF-induced angiogenesis. Proc. Nat. Acad. Sci. 99: 383-388, 2002).

Transgenic mice died in utero at embryonic day 8.5 when both NRP1 and NRP2, which they called Np1 and Np2, respectively, were knocked out. The yolk sacs of these mice were avascular. Mice deficient for NRP2 but heterozygous for NRP1 or deficient for NRP1 but heterozygous for NRP2 were also embryonic lethal and survived to embryonic days 10 to 10.5. Other details of the abnormal vascular phenotype resembled those of VEGF and Vefgr2 knockouts. The results suggested that neuropilins are early genes in embryonic vessel development and that both NRP1 and NRP2 are required (Takashima et al., Targeting of both mouse neuropilin-1 and neuropilin-2 genes severely impairs developmental yolk sac and embryonic angiogenesis. Proc. Nat. Acad. Sci. 99: 3657-3662, 2002).

It was shown in zebrafish the NRP1 protein was a functional receptor for human VEGF165. Whole-mount in situ hybridization showed that transcripts of the zebrafish NRP1 gene during embryonic and early larval development were detected mainly in neuronal and vascular tissues. A knockdown of the gene in embryos resulted in vascular defects. Embryos treated with VEGFR2 kinase inhibitor had a similar vessel defect, suggesting that knockdown of zebrafish NRP1 reduces VEGF activity. To determine whether NRP1 and VEGF activities are interdependent in vivo, zebrafish NRP1 and VEGF morpholinos were coinjected into embryos at concentrations that individually did not significantly inhibit blood vessel development. The result was a potent inhibition of blood cell circulation via both intersegmental and axial vessels, demonstrating that VEGF and NRP1 act synergistically to promote a functional circulatory system. These results provided the first physiologic demonstration that NRP1. regulated angiogenesis through a VEGF-dependent pathway (Lee et al., Neuropilin-1 is required for vascular development and is a mediator of VEGF-dependent angiogenesis in zebrafish. Proc. Nat. Acad. Sci. 99: 10470-10475, 2002).

NID2

The protein Nidogen 2 is also known as NID2 or osteonidogen. NID2 protein is 1376 amino acids in length and has a predicted molecular weight of 151,153 daltons (but see below). NID2 is a member of the nidogen superfamily and has EGF homology; LDL receptor YWTD-containing repeat homology; and thyroglobulin type I repeat homology. The NID2 genomic sequence has been mapped to chromosomal position 14q21-q22.

Basement membranes, which are composed of type IV collagens (see NCBI MIM 120130), laminins (see LAMC1; NCBI MIM 150290), perlecan (HSPG2; NCBI MIM 142461), and nidogen (see NID1; NCBI MIM 131390), are thin pericellular protein matrices that control a large number of cellular activities, including adhesion, migration, differentiation, gene expression, and apoptosis. By sequencing several overlapping cDNA clones in both directions, a cDNA encoding NID2 was obtained Kohfeldt et al., Nidogen-2: a new basement membrane protein with diverse binding properties. J. Molec. Biol. 282: 99-109, 1998). Sequence analysis predicted that the 1,375-amino acid NID2 protein, which is 46% identical to NID1, contains a 30-residue signal peptide, 49 primarily central cys residues, 5 potential N-linked glycosylation sites, 2 tyr residues in a potential O-sulfation sequence, and a YGD rather than an RGD cell adhesion sequence. Electron microscopy confirmed the presence of 3 deduced globular domains connected by a link and a rod-like region. Additional predicted structures similar to those found in NID1 include 5 epidermal growth factor (EGF; NCBI MIM 131530)-like modules, 2 of which have potential calcium-binding sequences; 2 thyroglobulin (NCBI MIM 188450) type I modules; and 5 low density lipoprotein (LDL) receptor (NCBI MIM 606945) modules. SDS-PAGE analysis showed that NID2 is expressed as a 200-kD protein, larger than the calculated mass of 148 kD, presumably due to oligosaccharide substitution as indicated by hexosamine analysis. Northern blot analysis revealed ubiquitous expression of a 5.5-kb NID2 transcript that was strongest in heart and placenta, moderate in pancreas, kidney, and skeletal muscle, and weakest in brain. Immunoblot analysis detected expression of NID2 in muscle, heart, placenta, kidney, skin, and testis, with weaker expression in liver and brain. Immunofluorescence analysis of mouse tissues showed staining of basement membranes usually in close colocalization with NID 1.

It was determined that the mouse NID2 gene contains 21 exons (Schymeinsky et al., Gene structure and functional analysis of the mouse nidogen-2 gene: nidogen-2 is not essential for basement membrane formation in mice. Molec. Cell. Biol. 22: 6820-6830, 2002). The promoter region contains several putative SP1 (NCBI MIM 189906) and CAAT recognition sites, but lacks a TATA box. Mice carrying a phenotypic null mutation in the NID2 gene have been developed. NID2-deficient mice showed no overt abnormalities, and their basement membranes appeared normal by ultrastructural analysis and immunostaining. NID2 deficiency did not lead to hemorrhages, and NID2 did not appear essential for basement membrane formation or maintenance.

ENDO180

The ENDO180 nucleotide sequence encodes a protein also known and sometimes referenced herein as UPARAP (urokinase plasminogen activator receptor-associated protein), KIAA0709, MRC2 (mannose receptor, C type 2), endocytic receptor (macrophage mannose receptor family) and is described as a novel mesenchymally expressed member of the macrophage mannose receptor family of endocytic receptors. ENDO180 is 1479 amino acids in length and has a molecular weight of about 180 kDa. ENDO180 has been mapped to chromosomal position 17q24.1.

ENDO180 is a member of the mannose receptor family (East et al., Biochim Biophys Acta. 2002 Sep. 19; 1572(2-3): 364-86. The mannose receptor family comprises four glycoproteins each of which is a type I transmembrane receptor with an N-terminal cysteine-rich domain, a single fibronectin type II (FNII) domain and eight to ten C-type lectin-like domains (CTLDs). Characteristically, these proteins are able to recycle between the plasma membrane and the endosomal apparatus due to discrete motifs present within their cytoplasmic domains. The family includes mannose receptor (MR), M-type receptor for secretory phospholipases A(2) (PLA(2)R), DEC-205/gp200-MR6 and ENDO180/uPARAP. Despite their overall structural similarity, these four receptors have evolved to use different domains to interact with discrete ligands. In addition, they differ in their ability to mediate endocytic and phagocytic events and in their intracellular destinations. Together, they represent a unique group of multidomain, multifunctional receptors.

Members of this receptor family are unusual in that they contain 8-10 C-type lectin carbohydrate recognition domains in a single polypeptide backbone, however, only the macrophage mannose receptor had been shown to function as a lectin (Sheikh et al., J Cell Sci. 2000 March; 113 (Pt 6): 1021-32). Sequence analysis of ENDO180 reveals that the second carbohydrate recognition domain has retained key conserved amino acids found in other functional C-type lectins. The protein also displays Ca(2+)-dependent binding to N-acetylglucosamine but not mannose affinity columns. ENDO180 predominantly is expressed in vivo and in vitro on fibroblasts, endothelial cells and macrophages, and the distribution and post-translational processing in these cells is consistent with ENDO180 functioning to internalize glycosylated ligands from the extracellular milieu for release in an endosomal compartment.

The uptake and lysosomal degradation of collagen by fibroblasts constitute a major pathway in the turnover of connective tissue (Engelholm, et al., J. Cell Biol. 2003 Mar. 31; 160(7): 1009-15). It was reported that ENDO180 is a key player in this process. Fibroblasts from mice with a targeted deletion in the ENDO180 gene displayed a near to complete abrogation of collagen endocytosis. These cells had diminished initial adhesion to a range of different collagens, as well as impaired migration on fibrillar collagen.

CDK10

The protein CDK10 (cyclin-dependent kinase (CDC2-like) 10) is also known as cyclin-dependent kinase 10 isoform 1 (331 amino acids); cyclin-dependent kinase 10 isoform 2 (314 amino acids); cyclin-dependent kinase 10 isoform 3 (123 amino acids); CDC2-related protein kinase; cell division protein kinase 10; cyclin-dependent kinase related protein; serine/threonine protein kinase and PISSLRE. CDK10 has been mapped to chromosomal position 16q24.

The protein encoded by CDK10 belongs to the CDK subfamily of the Ser/Thr protein kinase family. The CDK subfamily members are highly similar to the gene products of S. cerevisiae cdc28, and S. pombe cdc2, and are known to be essential for cell cycle progression. This kinase has been shown to play a role in cellular proliferation. Its function is limited to cell cycle G2-M phase. At least three alternatively spliced transcript variants encoding different isoforms have been reported, two of which contain multiple non-AUG translation initiation sites. Cyclin-dependent kinases (CDKs) are CDC2 (NCBI MIM 116940)-related kinases that bind to cyclin to form active holoenzymes that play a pivotal role in the regulation of the eukaryotic cell cycle. A 360-amino acid protein PISSLRE was predicted based on the amino acid sequence of the region corresponding to the conserved CDC2 PSTAIRE motif. PISSLRE contains all the structural elements characteristic of CDKs and unique extensions at both ends. Sequence comparisons revealed that it shares 41% and 50% protein sequence identity with CDC2 and CDC2L1 (NCBI MIM 176873), respectively. It was determined that PISSLRE was expressed broadly in human tissues as a 2-kb mRNA. An additional 3.5-kb transcript was observed in some tissues (Brambilla et al., Molecular cloning of PISSLRE, a novel putative member of the cdk family of protein serine/threonine kinases. Oncogene 9: 3037-3041, 1994).

FPGT

The protein FPGT (fucose-1-phosphate guanylyltransferase) is also known as GFPP and GDP-beta-L-fucose pyrophosphorylase. FPGT contains about 594 amino acids. FPGT has been mapped to chromosomal position 1p31.1.

L-fucose is a key sugar in glycoproteins and other complex carbohydrates since it may be involved in many of the functional roles of these macromolecules, such as in cell-cell recognition. The fucosyl donor for these fucosylated oligosaccharides is GDP-beta-L-fucose. There are two alternate pathways for the biosynthesis of GDP-fucose; the major pathway converts GDP-alpha-D-mannose to GDP-beta-L-fucose. FPGT participates in an alternate pathway that is present in certain mammalian tissues, such as liver and kidney, and appears to function as a salvage pathway to reutilize L-fucose arising from the turnover of glycoproteins and glycolipids. This pathway involves the phosphorylation of L-fucose to form beta-L-fucose-1-phosphate, and then condensation of the beta-L-fucose-1-phosphate with GTP by fucose-1-phosphate guanylyltransferase to form GDP-beta-L-fucose.

GFPP was purified from pig kidney and a partial protein sequence was determined. Human cDNAs encoding a region similar to one of the pig GFPP peptides also were identified. Using a PCR strategy with primers based on one of the ESTs, a cDNA corresponding to the entire human GFPP coding region was cloned. The predicted GFPP protein contains 594 amino acids. When expressed in mammalian cells, the human enzyme exhibited high levels of GFPP activity. Northern blot analysis indicated that the 3.5-kb GFPP mRNA is expressed in several human tissues (Pastuszak et al., GDP-L-fucose pyrophosphorylase: purification, cDNA cloning, and properties of the enzyme. J. Biol. Chem. 273: 30165-30174, 1998).

CARK

CARK is a cardiac-specific kinase gene localized on 1p31.1. Sequence analysis suggests that CARK is a distant family member of integrin-linked kinase. Northern blot and 76-tissue array analyses showed that CARK is highly expressed in heart, but is undetectable in other tissues. Immunohistochemical analysis predominantly localized CARK in the nucleus of cardiac myocytes. In vitro kinase assay showed that CARK is a functional kinase. The yeast two-hybrid system showed that CARK could directly interact with cardiac troponin I, results that were further confirmed by coimmunoprecipitation in vivo (Zhao Y et al. J Mol Med. 2003 May; 81(5): 297-304).

PCLO

The protein PCLO is also known as piccolo (presynaptic cytomatrix protein), ACZ, KIAA0559 and aczonin. PCLO contains about 1225 amino acids. PCLO has been mapped to chromosomal position 7q11.23-q21.1.

Synaptic vesicles dock and fuse in the active zone of the plasma membrane at chemical synapses. The presynaptic cytoskeletal matrix (PCM), which is associated with the active zone and is situated between synaptic vesicles, is thought to be involved in maintaining the neurotransmitter release site in register with the postsynaptic reception apparatus. The cycling of synaptic vesicles is a multistep process involving a number of proteins (see NCBI MIM 603215). Among the components of the PCM that orchestrate these events are Bassoon (BSN; NCBI MIM 604020), RIM (RBBP8; NCBI MIM 604124), Oboe, and Piccolo.

By screening human brain cDNAs for those encoding proteins larger than 50 kD, a partial cDNA encoding PCLO, referred to as KIAA0559, was identified. RT-PCR analysis detected PCLO expression in kidney, with little or no expression in all other tissues tested (Nagase et al., Prediction of the coding sequences of unidentified human genes. IX. The complete sequences of 100 new cDNA clones from brain which can code for large proteins in vitro. DNA Res. 5: 31-39, 1998).

By searching EST and genome databases with a murine Pclo cDNA probe, genomic sequences and a brain-specific EST (KIAA0559) encoding human PCLO were identified. Sequence analysis indicated that the deduced 4,880-amino acid rat Pclo protein is 86% identical to human PCLO. In addition, PCLO shares significant amino acid sequence homology with BSN. BSN and PCLO share 10 homology regions, or PBH regions. PBH1 and PBH2 contain 2 double-zinc finger motifs. PBH4, PBH6, and PBH8 are likely to form coiled-coil structures. At the C terminus, unlike BSN but like RIM and Oboe, PCLO contains a PDZ domain and a C2 domain. The PCLO C2 domain contains all the asp residues required for calcium binding. PCLO also contains multiple proline-rich segments. Confocal microscopy analysis of cultured hippocampal neurons showed colocalization of BSN and PCLO at identical GABAergic and glutamergic synapses, of synaptotagmin (see SYT1; NCBI MIM 185605) and PCLO along dendritic profiles, and of PCLO zinc fingers and PRA1 (NCBI MIM 604925) at nerve terminals (Fenster et al., Piccolo, a presynaptic zinc finger protein structurally related to Bassoon. Neuron 25: 203-214, 2000).

Using cAMP-GEFII (NCBI MIM 606058) as bait in a yeast 2-hybrid screen, mouse piccolo was cloned from an insulin-secreting cell line cDNA library. Northern blot analysis of mouse tissues revealed high levels in cerebrum and cerebellum and moderate levels in pituitary gland, pancreatic islets, and a pheochromocytoma-derived mouse cell line. In situ hybridization of mouse brain revealed piccolo mRNA expressed in cerebral cortex, hippocampus, olfactory bulb, cerebellar cortex, and pituitary gland. The distribution of piccolo mRNA largely overlapped that of cAMP-GEFII and Rim2 (NCBI MIM 606630) mRNA in tissues, cell lines, and mouse brain. Mouse piccolo interacts with both cAMP-GefI and Rim2. In the presence of Ca(2+), the C2A domain of piccolo could homodimerize, it could interact with the C2A domain of Rim2, or it could bind the cAMP GefII-Rim2 complex. It did not bind cAMP-GefII directly. Treatment of pancreatic islets with antisense piccolo oligonucleotides inhibited insulin secretion induced by a cAMP analog and high glucose stimulation. Piccolo serves as a Ca(2+) sensor in exocytosis in pancreatic beta cells and that the formation of a cAMP-GEFII-RIM2 piccolo complex is required (Fujimoto et al., Piccolo, a Ca(2+) sensor in pancreatic beta-cells: involvement of cAMP-GEFII-Rim2-piccolo complex in cAMP-dependent exocytosis. J. Biol. Chem. 277: 50497-50502, 2002).

By comparing the human PCLO genomic sequence with the rat Pclo cDNA, it was determined that the human PCLO gene contains at least 19 exons and spans over 350 kb.

REPS2

The protein REPS2 (RALBP1 associated Eps domain containing 2) is also known as POB1 and partner of Ral-binding protein 1. REPS2 contains about 521 amino acids. REPS2 has been mapped to chromosomal position Xp22.22.

Small G proteins have GDP-bound inactive and GTP-bound active forms; RAL proteins (e.g., RALA; NCBI MIM 179550) shift from the inactive to the active state through the actions of RALGDS (NCBI MIM 601619). RALGDS interacts with the active form of RAS (see HRAS; NCBI MIM 190020).

Using RALA-binding protein-1 (RALBP1; NCBI MIM 605801) as bait in a yeast 2-hybrid screen of a brain cDNA library, cDNAs encoding REP S2, which is designated POB 1, were identified (Ikeda et al., Identification and characterization of a novel protein interacting with Ral-binding protein 1, a putative effector protein of Ral. J. Biol. Chem. 273: 814-821, 1998). Sequence analysis predicted that the 521-amino acid protein has 2 potential initiator methionines in its N terminus, a central EPS 15 (NCBI MIM 600051)-like domain, and 2 proline-rich regions and a putative coiled-coil structure in its C terminus. Northern blot analysis revealed strong expression in rat cerebrum, cerebellum, lung, and testis, with weak expression in kidney and no expression in heart, thymus, liver, spleen, or adrenal gland. Immunoprecipitation and immunoblot analyses confirmed that the C-terminal 146 amino acids of REPS2 and the C-terminal 147 residues of RALBP 1 interact in intact cells. RAL interacts with a distinct region of RALBP 1, just N terminal of the REPS2-binding domain, and both proteins can interact simultaneously with RALBP1. Immunoblot analysis established that REPS2 is tyrosine phosphorylated in response to epidermal growth factor (EGF; NCBI MIM 131530) and interacts with the EGF receptor (EGFR; NCBI MIM 131550), possibly through the adaptor protein GRB2 (NCBI MIM 108355), with which REPS2 interacts specifically.

Using nuclear magnetic resonance spectroscopy, it was shown that the EPS 15 homology domain of REPS2 consists of 2 EF-hand structures, the second of which binds calcium (Koshiba et al., Solution structure of the Eps 15 homology domain of a human POB1 (partner of RalBP1). FEBS Lett. 442: 138-142, 1999).

As used herein, “polymorphic site” refers to a region in a nucleic acid at which two or more alternative nucleotide sequences are observed in a significant number of nucleic acid samples from a population of individuals. A polymorphic site may be a nucleotide sequence of two or more nucleotides, an inserted nucleotide or nucleotide sequence, a deleted nucleotide or nucleotide sequence, or a microsatellite, for example. A polymorphic site that is two or more nucleotides in length may be 3, 4, 5, 6, 7, 8, 9, 10, 11, 12, 13, 14, 15 or more, 20 or more, 30 or more, 50 or more, 75 or more, 100 or more, 500 or more, or about 1000 nucleotides in length, where all or some of the nucleotide sequences differ within the region. A polymorphic site is often one nucleotide in length, which is referred to herein as a “single nucleotide polymorphism” or a “SNP.”

Where there are two, three, or four alternative nucleotide sequences at a polymorphic site, each nucleotide sequence is referred to as a “polymorphic variant.” Where two polymorphic variants exist, for example, the polymorphic variant represented in aminority of samples from a population is sometimes referred to as a “minor allele” and the polymorphic variant that is more prevalently represented is sometimes referred to as a “major allele.” Many organisms possess a copy of each chromosome (e.g., humans), and those individuals who possess two major alleles or two minor alleles are often referred to as being “homozygous” with respect to the polymorphism, and those individuals who possess one major allele and one minor allele are normally referred to as being “heterozygous” with respect to the polymorphism. Individuals who are homozygous with respect to one allele are sometimes predisposed to a different phenotype as compared to individuals who are heterozygous or homozygous with respect to another allele. As shown hereafter, certain polymorphic variants of NRP1, NID2, ENDO180, CDK10, FPGT, CARK, PCLO or REPS2 nucleotide sequences are associated with melanoma.

A genotype or polymorphic variant may be expressed in terms of a “haplotype,” which as used herein refers to two or more polymorphic variants occurring within genomic DNA in a group of individuals within a population. For example, two SNPs may exist within a gene where each SNP position includes a cytosine variation and an adenine variation. Certain individuals in a population may carry one allele (heterozygous) or two alleles (homozygous) having the gene with a cytosine at each SNP position. As the two cytosines corresponding to each SNP in the gene travel together on one or both alleles in these individuals, the individuals can be characterized as having a cytosine/cytosine haplotype with respect to the two SNPs in the gene.

As used herein, “phenotype” refers to a trait which can be compared between individuals, such as presence or absence of a condition, a visually observable difference in appearance between individuals, metabolic variations, physiological variations, variations in the function of biological molecules, and the like. An example of a phenotype is occurrence of melanoma.

Researchers sometimes report a polymorphic variant in a database without determining whether the variant is represented in a significant fraction of a population. Because a subset of these reported polymorphic variants are not represented in a statistically significant portion of the population, some of them are sequencing errors and/or not biologically relevant. Thus, it often is not known whether a reported polymorphic variant is statistically significant or biologically relevant until the presence of the variant is detected in a population of individuals and the frequency of the variant is determined. Methods for detecting a polymorphic variant in a population are described herein, such as in Example 2. A polymorphic variant is statistically significant and often biologically relevant if it is represented in 5% or more of a population, sometimes 10% or more, 15% or more, or 20% or more of a population, and often 25% or more, 30% or more, 35% or more, 40% or more, 45% or more, or 50% or more of a population.

A polymorphic variant may be detected on either or both strands of a double-stranded nucleic acid. Also, a polymorphic variant may be located within an intron or exon of a gene or within a portion of a regulatory region such as a promoter, a 5′ untranslated region (UTR), a 3′ UTR, and in DNA (e.g., genomic DNA (gDNA) and complementary DNA (cDNA)), RNA (e.g., mRNA, tRNA, and rRNA), or a polypeptide. Polymorphic variations may or may not result in detectable differences in gene expression, polypeptide structure, or polypeptide function.

For duplex DNA, a polymorphic variation may be reported from one strand or its complementary strand. For example, a thymine at a particular position in SEQ ID NO: 1-17 can be reported as an adenine from the complementary strand. Also, while polymorphic variations at all positions within a haplotype often are reported from the same strand orientation, polymorphic variations at certain positions within a haplotype sometimes are reported from one strand orientation while others are reported from the other. The latter sometimes occurs even though it is understood by the person of ordinary skill in the art that polymorphic variants in a haplotype occur within one strand in a nucleic acid. Where a haplotype is reported from mixed strand orientations, a person of ordinary skill in the art can determine the orientation of each polymorphic variation in the haplotype by analyzing the orientation of each extension oligonucleotide utilized to identify each polymorphic variation.

In genetic analyses that associated polymorphic variations in NRP1, NID2, ENDO180, CDK10, FPGT, CARK, PCLO or REPS2 with melanoma, samples from individuals having melanoma and individuals not having cancer were allelotyped and genotyped. The term “allelotype” as used herein refers to a process for determining the allele frequency for a polymorphic variant in pooled DNA samples from cases and controls. By pooling DNA from each group, an allele frequency for each SNP in each group is calculated. These allele frequencies are then compared to one another. Particular SNPs are considered as being associated with a particular disease when allele frequency differences calculated between case and control pools are statistically significant. The terms “genotyped” and “genotyping” as used herein refer to a process for determining a genotype of one or more individuals, where a “genotype” is a representation of one or more polymorphic variants in a nucleic acid sample from an individual or a population. It was determined that SNPs associated with an increased risk of melanoma existed in NRP1, NID2, ENDO180, CDK10, FPGT, CARK, PCLO or REPS2 nucleotide sequences at rs1360457, rs753050, rs729647, rs8404, rs2034453, rs1044639 and rs1904528. Another polymorphic variation associated with an increased risk of melanoma was identified at rs3742536, which coded for a non-synonymous amino acid change at amino acid position 656 in a NID2 amino acid sequence (a proline or serine is encoded at this amino acid position with serine being associated with an increased risk of melanoma).

Polymorphic variants in and around the NRP1 region were tested for association with melanoma. These include polymorphic variants at positions selected from the group consisting of rs7910405, rs5784331, rs6481845, rs2065364, rs10643659, rs7896887, rs7086456, rs6481846, rs7475391, rs7477281, rs7475393, rs7475394, rs7477282, rs7475396, rs2768420, rs9418057, rs7070290, rs1360457, rs1411924, rs5784335, rs2776925, rs2776924, rs1952983, rs869636, rs869637, rs2804470, rs2776928, rs999966, rs2804475, rs4934858, rs4934863, rs4934864, rs1331326, rs1331325, rs4934871, rs4277057, rs2776930, rs1331324, rs2776932, rs4934896, rs2776933, rs4934901, rs1015300, rs2776934, rs2776935, rs2776936, rs2804491, rs2804492, rs2804493, rs2776937, rs2804494, rs4934914, rs2776922, rs2804495, rs2804496, rs4934597, rs2804497, rs2804498, rs2026319, rs1888684, rs3750733, rs4934927, rs2776946, rs4934599, rs4934934, rs2776944, rs2776943, rs2804886, rs2804900, rs2804902, rs2768397, rs2768399, rs2776941, rs2804888, rs2804499, rs2804889, rs2804891, rs2776940, rs2804897, rs2804896, rs2776939 rs2804894, rs2804501, rs2183715, rs2254822, rs2150686, rs2804893, rs2804502, rs2804892, rs2183714, rs2804503, rs1320485, rs4934603, rs2804890 and rs1854145. Polymorphic variants at the following positions in particular were associated with an increased risk of melanoma: rs6481845, rs10643659, rs7475391, rs7475394, rs7475396, rs9418057, rs1360457, rs5784335, rs1331325, rs4934871, rs2776930, rs2776935, rs2776936, rs2804491, rs2804494, rs2776922, rs2804495, rs2804496, rs1888684, rs2804888, rs2804891, rs2776940, rs2804897, rs2183714, and rs1854145. Tables in the Examples section hereafter show particular polymorphic variants indicative of a higher risk of melanoma in males and/or females. In specific embodiments, ppolymorphic variants at positions selected from the group consisting of rs7910405, rs5784331, rs6481845, rs2065364, rs10643659, rs7896887, rs7086456, rs6481846, rs7475391, rs7477281, rs7475393, rs7475394, rs7477282, rs7475396, rs2768420, rs9418057, rs7070290, rs1411924 and rs5784335 were tested for an association with increased risk of melanoma. Polymorphic variants at the following positions in particular were associated with an increased risk of melanoma: rs6481845, rs10643659, rs7475391, rs7475394, rs7475396, rs9418057, rs5784335. In particular, the following polymorphic variants were associated with risk of melanoma: a thymine at position rs6481845, a guanine-thymine-thymine-thymine deletion at position rs10643659, a cytosine at position rs7475391, a thymine at position rs7475394, a cytosine at position rs7475396, a thymine at position rs9418057 and a thymine at position rs5784335.

Variants in and around the ENDO180 region were tested for association with increased risk of melanoma. These include polymorphic variants at positions selected from the group consisting of rs2429393, rs2429392, rs2465421, rs2465410, rs2460302, rs2460301, rs2465459, rs2429391, rs2014055, rs2252814, rs8079582, rs2429390, rs7209331, rs7405815, rs4968706, rs4968613, rs729647, rs8072984, rs9896444, rs4968617, rs7350907, rs2429389, rs2460300, rs2465454, rs2465452, rs7219711, rs2465440, rs2429388, rs3786130, rs2460289, rs2465428, rs740698, rs2465425, rs2460290, rs3826537, rs3169756, rs1558237, rs2429385, rs5821368, rs8072538, rs2251393, rs2251391, rs2068657, rs8072519, rs7220505, rs2460288, rs4968736, position 16727 in SEQ ID NO: 3. Polymorphic variants at the following positions in particular were associated with an increased risk of melanoma: rs2429390, rs7209331, rs4968706, rs8072984, rs9896444, rs4968617, rs7350907, rs8072519. Tables in the Examples section hereafter show particular polymorphic variants indicative of a higher risk of melanoma in males and/or females. In particular, the following polymorphic variants were associated with risk of melanoma: a cytosine at position rs2429390, an adenine at position rs7209331, a cytosine at position rs4968706, a guanine at position rs729647, a guanine at position rs8072984, a thymine at position rs9896444, a cytosine at position rs4968617, a guanine at position rs7350907, a thymine at position rs8072519.

Polymorphic variants in and around the CDK10 region were tested for association with melanoma. These include polymorphic variants at positions selected from the group consisting of rs460879, rs460984, rs2437957, rs3815949, rs2437956, rs2437955, rs2434872, rs467357, rs258337, rs258336, rs258335, rs164752, rs154663, rs164753, rs258332, rs2010623, rs187283, rs258330, rs258328, rs459920, rs166297, rs258318, rs258317, rs171805, rs2081984, rs464586, rs460319, rs447735, rs2377058, rs604868, rs2118193, rs467035, rs2115401, rs417323, rs2377233, rs9746114, rs3751700, rs2277905, rs397891, rs3794638, rs3794637, rs258324, rs258323, rs2075880, rs258322, rs258321, rs164744, rs3833829, rs164743, rs164742, rs187282, rs465507, rs2162943, rs1946482, rs8404, rs1045814, rs4247353, rs462769, rs3803690, rs3751696, rs9972864, rs10567226, rs417414, rs154661, rs4785704, rs4785584, rs4785585, rs4785705, rs4785586, rs4785587, rs2016968, rs3751693, rs3809646, rs4785590, rs2099105, rs4785708, rs4785710, rs4785591, rs4785592, rs4785593, rs4785711, rs4785712, rs4785713, rs4785714, rs4785594, rs3803689, rs4785715, rs3764258, rs4785716, rs4785717, rs2077001, rs2377049, rs2003522, rs1230, rs1800359, rs1061646, rs1800358, rs2286392, rs2074904 and rs2074903. Polymorphic variants at the following positions in particular were associated with an increased risk of melanoma: rs460879, rs460984, rs3815949, rs2437955, rs258337, rs164752, rs164753, rs258318, rs464586, rs460319, rs2377233, rs2075880, rs258322, rs258321, rs164744, rs164743, rs164742, rs8404, rs417414, rs4785708, rs4785592, rs4785714, rs4785716 and rs1800359. Tables in the Examples section hereafter show particular polymorphic variants indicative of a higher risk of melanoma in males and/or females. In specific embodiments, polymorphic variants at positions selected from the group consisting of rs9746114, rs3833829, rs8404, rs1045814, rs3751696, rs9972864, rs10567226, position 36589 in SEQ ID NO: 4, position 44544 in SEQ ID NO: 4 and position 48304 in SEQ ID NO: 4. A polymorphic variant position rs8404, especially a cytosine at this position, was associated with an increased risk of melanoma.

Polymorphic variants in and around the FPGT and CARK regions were tested for association with melanoma. These include polymorphic variants at positions selected from the group consisting of rs1412825, rs4422957, rs3835390, rs944795, rs792310, rs526736, rs9804006, rs577367, rs575754, rs573721, rs487917, rs792307, rs476350, rs9425023, rs9425024, rs571848, rs489941, rs491623, rs956, rs520806, rs522759, rs545664, rs545721, rs536355, rs553044, rs581353, rs496008, rs7366854, rs560808, rs485929, rs477134, rs505634, rs542136, rs545680, rs522042, rs575961, rs792321, rs524306, rs495027, rs525358, rs471496, rs504866, rs532396, rs533371, rs567060, rs4650251, rs3753183, rs518769, rs474215, rs500203, rs532221, rs539534, rs531412, rs9970514, rs568049, rs9727250, rs792323, rs3765651, rs3765654, rs481387, rs483259, rs2034453, rs7536997, rs792324, rs6660350, rs492302, rs10493539, rs496720, rs570631, rs4593767, rs576802, rs518574, rs524252, rs792327, rs792328, rs2039407, rs503770, rs503904, rs514012, rs1412827, rs473834, rs480267, rs572180, rs1333029, rs485414, rs1412823, rs2027013, rs548881 and rs792329. Polymorphic variants at the following positions in particular were associated with an increased risk of melanoma: rs577367, rs487917, rs792307, rs489941, rs491623, rs536355, rs496008, rs560808, rs542136, rs524306, rs495027, rs504866, rs4650251, rs3753183, rs474215, rs500203, rs532221, rs531412, rs3765654, rs481387, rs483259, rs2034453, rs6660350, rs2039407, rs473834 and rs2027013. Tables in the Examples section hereafter show particular polymorphic variants indicative of a higher risk of melanoma in males and/or females. In specific embodiments, polymorphic variants at positions selected from the group consisting of rs3835390, rs9804006, rs9425023, rs9425024, rs489941, rs496008, rs7366854, rs4650251, rs539534, rs531412, rs9970514, rs568049, rs9727250, rs3765654, rs7536997, rs6660350, rs10493539, rs496720, position 206 in SEQ ID NO: 5, position 9261 in SEQ ID NO: 5, position 37592 in SEQ ID NO: 5. Polymorphic variants at the following positions in particular were associated with an increased risk of melanoma: rs489941, rs496008, rs4650251, rs531412, rs3765654, rs6660350, and position 206 in SEQ ID NO: 5. In particular, the following polymorphic variants were associated with risk of melanoma: an adenine at position rs489941, a thymine at position rs496008, an adenine at position rs4650251, an adenine at position rs531412, a cytosine at position rs3765654, a cytosine at position rs6660350, and a guanine at position 206 in SEQ ID NO: 5.

Polymorphic variants in and around the PCLO region were tested for association with melanoma. These include polymorphic variants at positions selected from the group consisting of rs2158220, rs1024465, rs4732483, rs4732485, rs1859176, rs1859177, rs2074401, rs4299948, rs4348435, rs4348436, rs4413718, rs985199, rs7791413, rs2522830, rs2214414, rs2189246, rs5885312, rs3064840, rs2051791, rs6467914, rs2214415, rs2189247, rs3064869, rs2522831, rs2715147, rs3064870, rs2715148, rs2522832, rs1044639, rs2715149, rs2522833, rs2247523, rs2371214, rs2189248, rs2715150, rs2715151, rs2371215, rs2715152, rs2715153, rs972346, rs2715154, rs2522834, rs2023847, rs2715155, rs2715156, rs10715043, rs2301722, rs2257207, rs2715157, rs2715158, rs2715159, rs2522835, rs2522836, rs2522837, rs2522838, rs2522839, rs2522840, rs2522841, rs10555175, rs10592258, rs2522842, rs6956848, rs2522843, rs2522844, rs2522845, rs2715161, rs6972720, rs4377905, rs4579453, rs4260846, rs1986481, rs4628206 and rs2888017. Polymorphic variants at the following positions in particular were associated with an increased risk of melanoma: rs1859176, rs2522830, rs2214414, rs2051791, rs6467914, rs2189247, rs3064869, rs2715148, rs1044639, rs2522833, rs2371214, rs2715151, rs2371215, rs2715153, rs2715154, rs2023847, rs2715155, rs2715156, rs2715157, rs2715158, rs2715159, rs2522835, rs2522837, rs10555175, rs6956848, rs2522845, rs4579453 and rs1986481. Tables in the Examples section hereafter show particular polymorphic variants indicative of a higher risk of melanoma in males and/or females. In specific embodiments, polymorphic variants at positions selected from the group consisting of rs7791413, rs2189246, rs5885312, rs3064840, rs6467914, rs3064869, rs3064870, rs10715043, rs2522841, rs10555175, rs10592258, rs6956848, rs6972720, position 53843 in SEQ ID NO: 6, position 63624 in SEQ ID NO: 6, position 65039 in SEQ ID NO: 6. Polymorphic variants at the following positions in particular were associated with an increased risk of melanoma: rs6467914, rs3064869, rs10555175, rs6956848, position 63624 in SEQ ID NO: 6, position 65039 in SEQ ID NO: 6. In particular, the following polymorphic variants were associated with risk of melanoma: a thymine at position rs6467914, an adenine at position rs3064869, a thymine at position rs10555175, a guanine at position rs6956848, position in SEQ ID NO: 6 selected from the group consisting of a guanine at position 63624 and an adenine-thymine insert at position 65039.

Polymorphic variants in and around the REPS2 region were tested for association with melanoma. These include polymorphic variants at positions selected from the group consisting of rs1904525, rs5924557, rs5924616, rs5924617, rs7883612, rs5924618, rs5924619, rs5901596, rs7878645, rs5924564, rs5924535, rs6629197, rs7060904, rs4828523, rs5924568, rs5924569, rs1904528, rs5924570, rs1904527, rs2052763, rs1549529, rs5924573, rs5901600, rs2288304, rs4240072, rs3055191, rs6632943, rs5924580, rs4828544, rs1807412, rs2010456, rs5969758, rs1965051, rs9887218, rs1426800, rs5969759, rs6527766, rs1365529, rs5924582, rs2114458, rs5924583, rs5924545, rs5969761, rs6629201, rs6629202, rs5924584, rs5924585, rs933946, rs5969762, rs713355, rs1365530, rs1961490, rs6527769, rs2006847, rs2382813, rs2033120, rs2162394, rs1346246, rs1365527, rs1426801, rs5924546, rs5924547, rs1365528, rs4828524, rs5924588, rs5969764, rs5924589, rs5924590, rs5924591, rs5924592, rs5924548, rs5924593, rs4828547, rs714070, rs5969768, rs4828526, rs5969738, rs5924595, rs5924549, rs4828528, rs5969769, rs4007744, rs2891073, rs5924599, rs1549531, rs5924601, rs5901609, rs5969739, rs5924602, rs1426798, rs7891150, rs4828550, rs5924603, rs1807947, rs954253, rs2382814, rs1896178, rs1896177, rs5924605, rs5924606, position 10537 in SEQ ID NO: 7, position 25216 in SEQ ID NO: 7 and position 119368 in SEQ ID NO: 7. Polymorphic variants at the following positions in particular were associated with an increased risk of melanoma: rs5924619, rs5924564, rs4828523, rs5924569, rs2052763, rs1549529, rs5924573, rs4240072, rs5924580, rs4828544, rs5969758, rs1965051, rs1426800, rs1365529, rs5924545, rs5969761, rs6629202, rs5924584, rs1365530, rs6527769, rs2382813, rs2033120, rs2162394, rs1346246, rs1365527, rs4828524, rs5924588, rs5969764, rs5924589, rs5924590, rs5924593, rs4828547, rs5969768, rs4828526, rs5924595, rs5924549, rs5969769, rs4007744, rs2891073, rs5924599, rs1549531, rs5924601, rs5901609, rs5969739, rs5924602, rs1426798, rs4828550, rs5924603, rs1807947, rs2382814, rs5924606, and position 25216 in SEQ ID NO: 7. Tables in the Examples section hereafter show particular polymorphic variants indicative of a higher risk of melanoma in males and/or females. In particular, the following polymorphic variants were associated with risk of melanoma: a thymine at position rs5924619, a cytosine at position rs5924564, a guanine at position rs4828523, an adenine at position rs5924569, a guanine at position rs2052763, an adenine at position rs1549529, an adenine at position rs5924573, an adenine at position rs4240072, a guanine at position rs5924580, a cytosine at position rs4828544, a guanine at position rs5969758, an adenine at position rs1965051, an adenine at position rs1426800, a cytosine at position rs1365529, an adenine at position rs5924545, a thymine at position rs5969761, a thymine at position rs6629202, a guanine at position rs5924584, a cytosine at position rs1365530, a cytosine at position rs6527769, a guanine at position rs2382813, a guanine at position rs2033120, an adenine at position rs2162394, a cytosine at position rs1346246, a cytosine at position rs1365527, an adenine at position rs4828524, a thymine at position rs5924588, a thymine at position rs5969764, a thymine at position rs5924589, a thymine at position rs5924590, a guanine at position rs5924593, a guanine at position rs4828547, a cytosine at position rs5969768, a guanine at position rs4828526, an adenine at position rs5924595, a guanine at position rs5924549, an adenine at position rs5969769, a thymine at position rs4007744, a thymine at position rs2891073, a guanine at position rs5924599, an adenine at position rs1549531, a guanine at position rs5924601, a cytosine at position rs5901609, a cytosine at position rs5969739, a thymine at position rs5924602, an adenine at position rs1426798, an adenine at position rs4828550, a thymine at position rs5924603, an adenine at position rs1807947, a thymine at position rs2382814, an adenine at position rs5924606, and an adenine at position 25216 in SEQ ID NO: 7.

The embodiments described hereafter may be directed to any of the polymorphic variants described herein. Sometimes, embodiments are directed to any of the polymorphic variants described herein with the proviso that the embodiments are not directed to a polymorphic variant at one or more positions selected from the group consisting of rs1360457, rs753050, rs729647, rs8404, rs2034453, rs1044639 and rs1904528.

Additional Polymorphic Variants Associated with Melanoma

Also provided is a method for identifying polymorphic variants proximal to an incident, founder polymorphic variant associated with melanoma. Thus, featured herein are methods for identifying a polymorphic variation associated with melanoma that is proximal to an incident polymorphic variation associated with melanoma, which comprises identifying a polymorphic variant proximal to the incident polymorphic variant associated with melanoma, where the incident polymorphic variant is in a nucleotide sequence set forth in SEQ ID NO: 1-17. The nucleotide sequence often comprises a polynucleotide sequence selected from the group consisting of (a) a polynucleotide sequence set forth in SEQ ID NO: 1-17; (b) a polynucleotide sequence that encodes a polypeptide having an amino acid sequence encoded by a nucleotide sequence set forth in SEQ ID NO: 1-17; and (c) a polynucleotide sequence that encodes a polypeptide having an amino acid sequence that is 90% or more identical to an amino acid sequence encoded by a nucleotide sequence set forth in SEQ ID NO: 1-17 or a polynucleotide sequence 90% or more identical to the polynucleotide sequence set forth in SEQ ID NO: 1-17. The presence or absence of an association of the proximal polymorphic variant with NIDDM then is determined using a known association method, such as a method described in the Examples hereafter. In an embodiment, the incident polymorphic variant is described in SEQ ID NO: 1-17 or in the Examples below. In another embodiment, the proximal polymorphic variant identified sometimes is a publicly disclosed polymorphic variant, which for example, sometimes is published in a publicly available database. In other embodiments, the polymorphic variant identified is not publicly disclosed and is discovered using a known method, including, but not limited to, sequencing a region surrounding the incident polymorphic variant in a group of nucleic samples. Thus, multiple polymorphic variants proximal to an incident polymorphic variant are associated with melanoma using this method.

The proximal polymorphic variant often is identified in a region surrounding the incident polymorphic variant. In certain embodiments, this surrounding region is about 50 kb flanking the first polymorphic variant (e.g about 50 kb 5′ of the first polymorphic variant and about 50 kb 3′ of the first polymorphic variant), and the region sometimes is composed of shorter flanking sequences, such as flanking sequences of about 40 kb, about 30 kb, about 25 kb, about 20 kb, about 15 kb, about 10 kb, about 7 kb, about 5 kb, or about 2 kb 5′ and 3′ of the incident polymorphic variant. In other embodiments, the region is composed of longer flanking sequences, such as flanking sequences of about 55 kb, about 60 kb, about 65 kb, about 70 kb, about 75 kb, about 80 kb, about 85 kb, about 90 kb, about 95 kb, or about 100 kb 5′ and 3′ of the incident polymorphic variant.

In certain embodiments, polymorphic variants associated with melanoma are identified iteratively. For example, a first proximal polymorphic variant is associated with melanoma using the methods described above and then another polymorphic variant proximal to the first proximal polymorphic variant is identified (e.g., publicly disclosed or discovered) and the presence or absence of an association of one or more other polymorphic variants proximal to the first proximal polymorphic variant with melanoma is determined.

The methods described herein are useful for identifying or discovering additional polymorphic variants that may be used to further characterize a gene, region or loci associated with a condition, a disease (e.g., melanoma), or a disorder. For example, allelotyping or genotyping data from the additional polymorphic variants may be used to identify a functional mutation or a region of linkage disequilibrium. In certain embodiments, polymorphic variants identified or discovered within a region comprising the first polymorphic variant associated with melanoma are genotyped using the genetic methods and sample selection techniques described herein, and it can be determined whether those polymorphic variants are in linkage disequilibrium with the first polymorphic variant. The size of the region in linkage disequilibrium with the first polymorphic variant also can be assessed using these genotyping methods. Thus, provided herein are methods for determining whether a polymorphic variant is in linkage disequilibrium with a first polymorphic variant associated with melanoma, and such information can be used in prognosis methods described herein.

Isolated Nucleic Acids and Variants Thereof

Featured herein are isolated NRP1, NID2, ENDO180, CDK10, FPGT, CARK, PCLO or REPS2 nucleic acids, which include the nucleotide sequence of SEQ ID NO: 1-17, NRP1, NID2, ENDO180, CDK10, FPGT, CARK, PCLO or REPS2 nucleic acid variants, and substantially identical nucleic acids and fragments of the foregoing. Nucleotide sequences of NRP1, NID2, ENDO180, CDK10, FPGT, CARK, PCLO or REPS2 nucleic acids sometimes are referred to herein as “NRP1, NID2, ENDO180, CDK10, FPGT, CARK, PCLO or REPS2 nucleotide sequences.” A “NRP1, NID2, ENDO180, CDK10, FPGT, CARK, PCLO or REPS2 nucleic acid variant” refers to one allele that may have one or more different polymorphic variations as compared to another allele in another subject or the same subject. A polymorphic variation in the NRP1, NID2, ENDO180, CDK10, FPGT, CARK, PCLO or REPS2 nucleic acid variant may be represented on one or both strands in a double-stranded nucleic acid or on one chromosomal complement (heterozygous) or both chromosomal complements (homozygous). A NRP1, NID2, ENDO180, CDK10, FPGT, CARK, PCLO or REPS2 nucleic acid may comprise one or more polymorphic variations associated with melanoma described herein.

As used herein, the term “nucleic acid” includes DNA molecules (e.g., a complementary DNA (cDNA) and genomic DNA (gDNA)) and RNA molecules (e.g., mRNA, rRNA, siRNA and tRNA) and analogs of DNA or RNA, for example, by use of nucleotide analogs. The nucleic acid molecule can be single-stranded and it is often double-stranded. The term “isolated or purified nucleic acid” refers to nucleic acids that are separated from other nucleic acids present in the natural source of the nucleic acid. For example, with regard to genomic DNA, the term “isolated” includes nucleic acids which are separated from the chromosome with which the genomic DNA is naturally associated. An “isolated” nucleic acid is often free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. As used herein, the term “NRP1, NID2, ENDO180, CDK10, FPGT, CARK, PCLO or REPS2 gene” refers to a nucleotide sequence that encodes a NRP1, NID2, ENDO180, CDK10, FPGT, CARK, PCLO or REPS2 polypeptide.

In particular embodiments, a nucleic acid comprises one or more polymorphic variations independently selected from the group consisting of an adenine at position 16727 in SEQ ID NO: 3, a guanine at position 36589 in SEQ ID NO: 4, a thymine at position 44544 in SEQ ID NO: 4, a guanine at position 48304 in SEQ ID NO: 4, a guanine at position 53843 in SEQ ID NO: 6, a guanine at position 63624 in SEQ ID NO: 6, an adenine-thymine deletion at position 65039 in SEQ ID NO: 6, a guanine at position 206 in SEQ ID NO: 5, an adenine at position 9261 in SEQ ID NO: 5, a thymine at position 37592 in SEQ ID NO: 5, a guanine at position 10537 in SEQ ID NO: 7, a cytosine at position 25216 in SEQ ID NO: 7 and a thymine at position 119368 in SEQ ID NO: 7. The nucleic acid often comprises a part of or all of a nucleotide sequence in SEQ ID NO: 3, 4, 5, 6 and/or 7, and sometimes such a nucleotide sequence is a 5′ and/or 3′ sequence flanking a polymorphic variant described above that is 5, 6, 7 . . . 50, 51, 52 . . . 100, 101, 102 . . . 500, 501, 502 . . . 999 or 1000 nucleotides in length. Other embodiments are directed to methods of identifying a polymorphic variation at one or more positions in a nucleic acid (e.g., genotyping at one or more positions in the nucleic acid), where the one or more positions are independently selected from the group consisting of position 16727 in SEQ ID NO: 3, position 36589 in SEQ ID NO: 4, position 44544 in SEQ ID NO: 4, position 48304 in SEQ ID NO: 4, position 53843 in SEQ ID NO: 6, position 63624 in SEQ ID NO: 6, position 65039 in SEQ ID NO: 6, position 206 in SEQ ID NO: 5, position 9261 in SEQ ID NO: 5, position 37592 in SEQ ID NO: 5, position 10537 in SEQ ID NO: 7, position 25216 in SEQ ID NO: 7 and position 119368 in SEQ ID NO: 7.

Also included herein are nucleic acid fragments. These fragments are typically a nucleotide sequence identical to a nucleotide sequence in SEQ ID NO: 1-17, a nucleotide sequence substantially identical to a nucleotide sequence in SEQ ID NO: 1-17, or a nucleotide sequence that is complementary to the foregoing. The nucleic acid fragment may be identical, substantially identical or homologous to a nucleotide sequence in an exon or an intron in SEQ ID NO: 1-17 and may encode a full-length or mature polypeptide, or may encode a domain or part of a domain of a NRP1, NID2, ENDO180, CDK10, FPGT, CARK, PCLO or REPS2 polypeptide. Sometimes, the fragment will comprises one or more of the polymorphic variations described herein as being associated with melanoma. The nucleic acid fragment is often 50, 100, or 200 or fewer base pairs in length, and is sometimes about 300, 400, 500, 600, 700, 800, 900, 1000, 1100, 1200, 1300, or 1400 base pairs in length. A nucleic acid fragment that is complementary to a nucleotide sequence identical or substantially identical to the nucleotide sequence of SEQ ID NO: 1-17 and hybridizes to such a nucleotide sequence under stringent conditions is often referred to as a “probe.” Nucleic acid fragments often include one or more polymorphic sites, or sometimes have an end that is adjacent to a polymorphic site as described hereafter.

An example of a nucleic acid fragment is an oligonucleotide. As used herein, the term “oligonucleotide” refers to a nucleic acid comprising about 8 to about 50 covalently linked nucleotides, often comprising from about 8 to about 35 nucleotides, and more often from about 10 to about 25 nucleotides. The backbone and nucleotides within an oligonucleotide may be the same as those of naturally occurring nucleic acids, or analogs or derivatives of naturally occurring nucleic acids, provided that oligonucleotides having such analogs or derivatives retain the ability to hybridize specifically to a nucleic acid comprising a targeted polymorphism. Oligonucleotides described herein may be used as hybridization probes or as components of prognostic or diagnostic assays, for example, as described herein.

Oligonucleotides are typically synthesized using standard methods and equipment, such as the ABI™3900 High Throughput DNA Synthesizer and the EXPEDITE™ 8909 Nucleic Acid Synthesizer, both of which are available from Applied Biosystems (Foster City, Calif.). Analogs and derivatives are exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; WO 00/56746; WO 01/14398, and related publications. Methods for synthesizing oligonucleotides comprising such analogs or derivatives are disclosed, for example, in the patent publications cited above and in U.S. Pat. Nos. 5,614,622; 5,739,314; 5,955,599; 5,962,674; 6,117,992; in WO 00/75372; and in related publications.

Oligonucleotides may also be linked to a second moiety. The second moiety may be an additional nucleotide sequence such as a tail sequence (e.g., a polyadenosine tail), an adapter sequence (e.g., phage M13 universal tail sequence), and others. Alternatively, the second moiety may be a non-nucleotide moiety such as a moiety which facilitates linkage to a solid support or a label to facilitate detection of the oligonucleotide. Such labels include, without limitation, a radioactive label, a fluorescent label, a chemiluminescent label, a paramagnetic label, and the like. The second moiety may be attached to any position of the oligonucleotide, provided the oligonucleotide can hybridize to the nucleic acid comprising the polymorphism.

Uses for Nucleic Acids

Nucleic acid coding sequences depicted in SEQ ID NO: 1-17, or substantially identical sequences thereof, may be used for diagnostic purposes for detection and control of polypeptide expression. Also, included are oligonucleotide sequences such as antisense nucleic acids (e.g., DNA, RNA or PNA), inhibitory RNA and small-interfering RNA (siRNA), and ribozymes that function to inhibit translation of a polypeptide. Antisense techniques and RNA interference techniques are known in the art and are described herein.

Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. For example, hammerhead motif ribozyme molecules may be engineered that specifically and efficiently catalyze endonucleolytic cleavage of RNA sequences encoded by a nucleotide sequence set forth in SEQ ID NO: 1-17 or a substantially identical sequence thereof. Specific ribozyme cleavage sites within any potential RNA target are initially identified by scanning the target molecule for ribozyme cleavage sites which include the following sequences, GUA, GUU and GUC. Once identified, short RNA sequences of between fifteen (15) and twenty (20) ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

Antisense RNA and DNA molecules, siRNA and ribozymes may be prepared by any method known in the art for the synthesis of RNA molecules. These include techniques for chemically synthesizing oligodeoxyribonucleotides well known in the art such as solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

DNA encoding a polypeptide also may have a number of uses for the diagnosis of diseases, including melanoma, resulting from aberrant expression of a target gene described herein. For example, the nucleic acid sequence may be used in hybridization assays of biopsies or autopsies to diagnose abnormalities of expression or function (e.g., Southern or Northern blot analysis, in situ hybridization assays).

In addition, the expression of a polypeptide during embryonic development may also be determined using nucleic acid encoding the polypeptide. As addressed, infra, production of functionally impaired polypeptide is the cause of various disease states, melanoma. In situ hybridizations using polypeptide as a probe may be employed to predict problems related to melanoma. Further, as indicated, infra, administration of human active polypeptide, recombinantly produced as described herein, may be used to treat disease states related to functionally impaired polypeptide. Alternatively, gene therapy approaches may be employed to remedy deficiencies of functional polypeptide or to replace or compete with dysfunctional polypeptide.

Expression Vectors, Host Cells, and Genetically Engineered Cells

Provided herein are nucleic acid vectors, often expression vectors, which contain a NRP1, NID2, ENDO180, CDK10, FPGT, CARK, PCLO or REPS2 nucleic acid. As used herein, the term “vector” refers to a nucleic acid molecule capable of transporting another nucleic acid to which it has been linked and can include a plasmid, cosmid, or viral vector. The vector can be capable of autonomous replication or it can integrate into a host DNA. Viral vectors may include replication defective retroviruses, adenoviruses and adeno-associated viruses for example.

A vector can include a NRP1, NID2, ENDO180, CDK10, FPGT, CARK, PCLO or REPS2 nucleic acid in a form suitable for expression of the nucleic acid in a host cell. The recombinant expression vector typically includes one or more regulatory sequences operatively linked to the nucleic acid sequence to be expressed. The term “regulatory sequence” includes promoters, enhancers and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of polypeptide desired, and the like. Expression vectors can be introduced into host cells to produce NRP1, NID2, ENDO180, CDK10, FPGT, CARK, PCLO or REPS2 polypeptides, including fusion polypeptides, encoded by NRP1, NID2, ENDO180, CDK10, FPGT, CARK, PCLO or REPS2 nucleic acids.

Recombinant expression vectors can be designed for expression of NRP1, NID2, ENDO180, CDK10, FPGT, CARK, PCLO or REPS2 polypeptides in prokaryotic or eukaryotic cells. For example, NRP1, NID2, ENDO180, CDK10, FPGT, CARK, PCLO or REPS2 polypeptides can be expressed in E. coli, insect cells (e.g., using baculovirus expression vectors), yeast cells, or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of polypeptides in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: 1) to increase expression of recombinant polypeptide; 2) to increase the solubility of the recombinant polypeptide; and 3) to aid in the purification of the recombinant polypeptide by acting as a ligand in affinity purification. Often, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith & Johnson, Gene 67: 31-40 (1988)), pMAL (New England Biolabs, Beverly, Mass.) and pRIT5 (Pharmacia, Piscataway, N.J.) which fuse glutathione S-transferase (GST), maltose E binding polypeptide, or polypeptide A, respectively, to the target recombinant polypeptide.

Purified fusion polypeptides can be used in screening assays and to generate antibodies specific for NRP1, NID2, ENDO180, CDK10, FPGT, CARK, PCLO or REPS2 polypeptides. In a therapeutic embodiment, fusion polypeptide expressed in a retroviral expression vector is used to infect bone marrow cells that are subsequently transplanted into irradiated recipients. The pathology of the subject recipient is then examined after sufficient time has passed (e.g., six (6) weeks).

Expressing the polypeptide in host bacteria with an impaired capacity to proteolytically cleave the recombinant polypeptide is often used to maximize recombinant polypeptide expression (Gottesman, S., Gene Expression Technology: Methods in Enzymology, Academic Press, San Diego, Calif. 185: 119-128 (1990)). Another strategy is to alter the nucleotide sequence of the nucleic acid to be inserted into an expression vector so that the individual codons for each amino acid are those preferentially utilized in E. coli (Wada et al., Nucleic Acids Res. 20: 2111-2118 (1992)). Such alteration of nucleotide sequences can be carried out by standard DNA synthesis techniques.

When used in mammalian cells, the expression vector's control functions are often provided by viral regulatory elements. For example, commonly used promoters are derived from polyoma, Adenovirus 2, cytomegalovirus and Simian Virus 40. Recombinant mammalian expression vectors are often capable of directing expression of the nucleic acid in a particular cell type (e.g., tissue-specific regulatory elements are used to express the nucleic acid). Non-limiting examples of suitable tissue-specific promoters include an albumin promoter (liver-specific; Pinkert et al., Genes Dev. 1: 268-277 (1987)), lymphoid-specific promoters (Calame & Eaton, Adv. Immunol. 43: 235-275 (1988)), promoters of T cell receptors (Winoto & Baltimore, EMBO J. 8: 729-733 (1989)) promoters of immunoglobulins (Banerji et al., Cell 33: 729-740 (1983); Queen & Baltimore, Cell 33: 741-748 (1983)), neuron-specific promoters (e.g., the neurofilament promoter; Byrne & Ruddle, Proc. Natl. Acad. Sci. USA 86: 5473-5477 (1989)), pancreas-specific promoters (Edlund et al., Science 230: 912-916 (1985)), and mammary gland-specific promoters (e.g., milk whey promoter; U.S. Pat. No. 4,873,316 and European Application Publication No. 264,166). Developmentally-regulated promoters are sometimes utilized, for example, the murine hox promoters (Kessel & Gruss, Science 249: 374-379 (1990)) and the a-fetopolypeptide promoter (Campes & Tilghman, Genes Dev. 3: 537-546 (1989)).

A NRP1, NID2, ENDO180, CDK10, FPGT, CARK, PCLO or REPS2 nucleic acid may also be cloned into an expression vector in an antisense orientation. Regulatory sequences (e.g., viral promoters and/or enhancers) operatively linked to a NRP1, NID2, ENDO180, CDK10, FPGT, CARK, PCLO or REPS2 nucleic acid cloned in the antisense orientation can be chosen for directing constitutive, tissue specific or cell type specific expression of antisense RNA in a variety of cell types. Antisense expression vectors can be in the form of a recombinant plasmid, phagemid or attenuated virus. For a discussion of the regulation of gene expression using antisense genes see Weintraub et al., Antisense RNA as a molecular tool for genetic analysis, Reviews—Trends in Genetics, Vol. 1(1) (1986).

Also provided herein are host cells that include a NRP1, NID2, ENDO180, CDK10, FPGT, CARK, PCLO or REPS2 nucleic acid within a recombinant expression vector or NRP1, NID2, ENDO180, CDK10, FPGT, CARK, PCLO or REPS2 nucleic acid sequence fragments which allow it to homologously recombine into a specific site of the host cell genome. The terms “host cell” and “recombinant host cell” are used interchangeably herein. Such terms refer not only to the particular subject cell but rather also to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, a NRP1, NID2, ENDO180, CDK10, FPGT, CARK, PCLO or REPS2 polypeptide can be expressed in bacterial cells such as E. coli, insect cells, yeast or mammalian cells (such as Chinese hamster ovary cells (CHO) or COS cells). Other suitable host cells are known to those skilled in the art.

Vectors can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, transduction/infection, DEAE-dextran-mediated transfection, lipofection, or electroporation.

A host cell provided herein can be used to produce (i.e., express) a NRP1, NID2, ENDO180, CDK10, FPGT, CARK, PCLO or REPS2 polypeptide. Accordingly, further provided are methods for producing a NRP1, NID2, ENDO180, CDK10, FPGT, CARK, PCLO or REPS2 polypeptide using the host cells described herein. In one embodiment, the method includes culturing host cells into which a recombinant expression vector encoding a NRP1, NID2, ENDO180, CDK10, FPGT, CARK, PCLO or REPS2 polypeptide has been introduced in a suitable medium such that a NRP1, NID2, ENDO180, CDK10, FPGT, CARK, PCLO or REPS2 polypeptide is produced. In another embodiment, the method further includes isolating a NRP1, NID2, ENDO180, CDK10, FPGT, CARK, PCLO or REPS2 polypeptide from the medium or the host cell.

Also provided are cells or purified preparations of cells which include a NRP1, NID2, ENDO180, CDK10, FPGT, CARK, PCLO or REPS2 transgene, or which otherwise misexpress NRP1, NID2, ENDO180, CDK10, FPGT, CARK, PCLO or REPS2 polypeptide. Cell preparations can consist of human or non-human cells, e.g., rodent cells, e.g., mouse or rat cells, rabbit cells, or pig cells. In embodiments, the cell or cells include a NRP1, NID2, ENDO180, CDK10, FPGT, CARK, PCLO or REPS2 transgene (e.g., a heterologous form of a NRP1, NID2, ENDO180, CDK10, FPGT, CARK, PCLO or REPS2 such as a human gene expressed in non-human cells). The NRP1, NID2, ENDO180, CDK10, FPGT, CARK, PCLO or REPS2 transgene can be misexpressed, e.g., overexpressed or underexpressed. In other embodiments, the cell or cells include a gene which misexpress an endogenous NRP1, NID2, ENDO180, CDK10, FPGT, CARK, PCLO or REPS2 polypeptide (e.g., expression of a gene is disrupted, also known as a knockout). Such cells can serve as a model for studying disorders which are related to mutated or mis-expressed NRP1, NID2, ENDO180, CDK10, FPGT, CARK, PCLO or REPS2 alleles or for use in drug screening. Also provided are human cells (e.g., a hematopoietic stem cells) transformed with a NRP1, NID2, ENDO180, CDK10, FPGT, CARK, PCLO or REPS2 nucleic acid.

Also provided are cells or a purified preparation thereof (e.g., human cells) in which an endogenous NRP1, NID2, ENDO180, CDK10, FPGT, CARK, PCLO or REPS2 nucleic acid is under the control of a regulatory sequence that does not normally control the expression of the endogenous NRP1, NID2, ENDO180, CDK10, FPGT, CARK, PCLO or REPS2 gene. The expression characteristics of an endogenous gene within a cell (e.g., a cell line or microorganism) can be modified by inserting a heterologous DNA regulatory element into the genome of the cell such that the inserted regulatory element is operably linked to the endogenous NRP1, NID2, ENDO180, CDK10, FPGT, CARK, PCLO or REPS2 gene. For example, an endogenous NRP1, NID2, ENDO180, CDK10, FPGT, CARK, PCLO or REPS2 gene (e.g., a gene which is “transcriptionally silent,” not normally expressed, or expressed only at very low levels) may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published on May 16, 1991.

Transgenic Animals

Non-human transgenic animals that express a heterologous NRP1, NID2, ENDO180, CDK10, FPGT, CARK, PCLO or REPS2 polypeptide (e.g., expressed from a NRP1, NID2, ENDO180, CDK10, FPGT, CARK, PCLO or REPS2 nucleic acid isolated from another organism) can be generated. Such animals are useful for studying the function and/or activity of a NRP1, NID2, ENDO180, CDK10, FPGT, CARK, PCLO or REPS2 polypeptide and for identifying and/or evaluating modulators of NRP1, NID2, ENDO180, CDK10, FPGT, CARK, PCLO or REPS2 nucleic acid and NRP1, NID2, ENDO180, CDK10, FPGT, CARK, PCLO or REPS2 polypeptide activity. As used herein, a “transgenic animal” is a non-human animal such as a mammal (e.g., a non-human primate such as chimpanzee, baboon, or macaque; an ungulate such as an equine, bovine, or caprine; or a rodent such as a rat, a mouse, or an Israeli sand rat), a bird (e.g., a chicken or a turkey), an amphibian (e.g., a frog, salamander, or newt), or an insect (e.g., Drosophila melanogaster), in which one or more of the cells of the animal includes a NRP1, NID2, ENDO180, CDK10, FPGT, CARK, PCLO or REPS2 transgene. A transgene is exogenous DNA or a rearrangement (e.g., a deletion of endogenous chromosomal DNA) that is often integrated into or occurs in the genome of cells in a transgenic animal. A transgene can direct expression of an encoded gene product in one or more cell types or tissues of the transgenic animal, and other transgenes can reduce expression (e.g., a knockout). Thus, a transgenic animal can be one in which an endogenous NRP1, NID2 ENDO180, CDK10, FPGT, CARK, PCLO or REPS2 gene has been altered by homologous recombination between the endogenous gene and an exogenous DNA molecule introduced into a cell of the animal (e.g., an embryonic cell of the animal) prior to development of the animal.

Intronic sequences and polyadenylation signals can also be included in the transgene to increase expression efficiency of the transgene. One or more tissue-specific regulatory sequences can be operably linked to a NRP1, NID2, ENDO180, CDK10, FPGT, CARK, PCLO or REPS2 transgene to direct expression of a NRP1, NID2 ENDO180, CDK10, FPGT, CARK, PCLO or REPS2 polypeptide to particular cells. A transgenic founder animal can be identified based upon the presence of a NRP1, NID2, ENDO180, CDK10, FPGT, CARK, PCLO or REPS2 transgene in its genome and/or expression of NRP1, NID2, ENDO180, CDK10, FPGT, CARK, PCLO or REPS2 mRNA in tissues or cells of the animals. A transgenic founder animal can then be used to breed additional animals carrying the transgene. Moreover, transgenic animals carrying a transgene encoding a NRP1, NID2, ENDO180, CDK10, FPGT, CARK, PCLO or REPS2 polypeptide can further be bred to other transgenic animals carrying other transgenes.

NRP1, NID2, ENDO180, CDK10, FPGT, CARK, PCLO or REPS2 polypeptides can be expressed in transgenic animals or plants by introducing, for example, a nucleic acid encoding the polypeptide into the genome of an animal. In embodiments the nucleic acid is placed under the control of a tissue specific promoter, e.g., a milk or egg specific promoter, and recovered from the milk or eggs produced by the animal. Also included is a population of cells from a transgenic animal.

NRP1. NID2. ENDO180. CDK10. FPGT. CARK, PCLO or REPS2 Polypeptides

Also featured herein are isolated NRP1, NID2, ENDO180, CDK10, FPGT, CARK, PCLO or REPS2 polypeptides, including proteins and peptides, that include an amino acid sequence set forth in SEQ ID NO: 18-27, or a substantially identical sequence thereof or variant thereof. In an embodiment, an NID2 polypeptide includes a serine at amino acid position 656. Isolated NRP1, NID2 ENDO180, CDK10, FPGT, CARK, PCLO or REPS2 polypeptides featured herein include both the full-length polypeptide and the mature polypeptide (i.e., the polypeptide minus the signal sequence or propeptide domain). A NRP1, NID2, ENDO180, CDK10, FPGT, CARK, PCLO or REPS2 polypeptide is a polypeptide encoded by a NRP1, NID2, ENDO180, CDK10, FPGT, CARK, PCLO or REPS2 nucleic acid, where one nucleic acid can encode one or more different polypeptides. An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of a NRP1, NID2, ENDO180, CDK10, FPGT, CARK, PCLO or REPS2 polypeptide or NRP1, NID2, ENDO180, CDK10, FPGT, CARK, PCLO or REPS2 polypeptide variant having less than about 30%, 20%, 10% and more preferably 5% (by dry weight), of non-NRP1, NID2, ENDO180, CDK10, FPGT, CARK, PCLO or REPS2 polypeptide (also referred to herein as a “contaminating protein”), or of chemical precursors or non-NRP1, NID2, ENDO180, CDK10, FPGT, CARK, PCLO or REPS2 chemicals. When the NRP1, NID2, ENDO180, CDK10, FPGT, CARK, PCLO or REPS2 polypeptide or a biologically active portion thereof is recombinantly produced, it is also preferably substantially free of culture medium, specifically, where culture medium represents less than about 20%, sometimes less than about 10%, and often less than about 5% of the volume of the polypeptide preparation. Isolated or purified NRP1, NID2, ENDO180, CDK10, FPGT, CARK, PCLO or REPS2 polypeptide preparations are sometimes 0.01 milligrams or more or 0.1 milligrams or more, and often 1.0 milligrams or more and 10 milligrams or more in dry weight.

Further included herein are NRP1, NID2, ENDO180, CDK10, FPGT, CARK, PCLO or REPS2 polypeptide fragments. The polypeptide fragment may be a domain or part of a domain of a NRP1, NID2, ENDO180, CDK10, FPGT, CARK PCLO or REPS2 polypeptide. The polypeptide fragment may have increased, decreased or unexpected biological activity. The polypeptide fragment is often 50 or fewer, 100 or fewer, or 200 or fewer amino acids in length, and is sometimes 300, 400, 500, 600, or 700, or fewer amino acids in length. A fragment sometimes includes a substantial portion of a domain, examples of which are described herein.

Substantially identical polypeptides may depart from the amino acid sequences set forth in SEQ ID NO: 18-27 in different manners. For example, conservative amino acid modifications may be introduced at one or more positions in the amino acid sequences of SEQ ID NO: 18-27. A “conservative amino acid substitution” is one in which the amino acid is replaced by another amino acid having a similar structure and/or chemical function. Families of amino acid residues having similar structures and functions are well known. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Also, essential and non-essential amino acids may be replaced. A “non-essential” amino acid is one that can be altered without abolishing or substantially altering the biological function of a NRP1, NID2, ENDO180, CDK10, FPGT, CARK, PCLO or REPS2 polypeptide, whereas altering an “essential” amino acid abolishes or substantially alters the biological function of a NRP1, NID2, ENDO180, CDK10, FPGT, CARK, PCLO or REPS2 polypeptide. Amino acids that are conserved among NRP1, NID2, ENDO180, CDK10, FPGT, CARK, PCLO or REPS2 polypeptides are typically essential amino acids.

Also, NRP1, NID2, ENDO180, CDK10, FPGT, CARK PCLO or REPS2 polypeptides and polypeptide variants may exist as chimeric or fusion polypeptides. As used herein, a NRP1, NID2, ENDO180, CDK10, FPGT, CARK, PCLO or REPS2 “chimeric polypeptide” or “fusion polypeptide” includes a NRP1, NID2, ENDO180, CDK10, FPGT, CARK, PCLO or REPS2 polypeptide linked to a non-NRP1, NID2, ENDO180, CDK10, FPGT, CARK, PCLO or REPS2 polypeptide. A “non-NRP1, NID2, ENDO180, CDK10, FPGT, CARK, PCLO or REPS2 polypeptide” refers to a polypeptide having an amino acid sequence corresponding to a polypeptide which is not substantially identical to the NRP1, NID2, ENDO180, CDK10, FPGT, CARK, PCLO or REPS2 polypeptide, which includes, for example, a polypeptide that is different from the NRP1, NID2, ENDO180, CDK10, FPGT, CARK, PCLO or REPS2 polypeptide and derived from the same or a different organism. The NRP1, NID2, ENDO180, CDK10, FPGT, CARK, PCLO or REPS2 polypeptide in the fusion polypeptide can correspond to an entire or nearly entire NRP1, NID2, ENDO180, CDK10, FPGT, CARK, PCLO or REPS2 polypeptide or a fragment thereof. The non-NRP1, NID2, ENDO180, CDK10, FPGT, CARK, PCLO or REPS2 polypeptide can be fused to the N-terminus or C-terminus of the NRP1, NID2, ENDO180, CDK10, FPGT, CARK, PCLO or REPS2 polypeptide.

Fusion polypeptides can include a moiety having high affinity for a ligand. For example, the fusion polypeptide can be a GST-NRP1, NID2, ENDO180, CDK10, FPGT, CARK, PCLO or REPS2 fusion polypeptide in which the NRP1, NID2, ENDO180, CDK10, FPGT, CARK, PCLO or REPS2 sequences are fused to the C-terminus of the GST sequences, or a polyhistidine-NRP1, NID2, ENDO180, CDK10, FPGT, CARK, PCLO or REPS2 fusion polypeptide in which the NRP1, NID2 ENDO180, CDK10, FPGT, CARK, PCLO or REPS2 polypeptide is fused at the N- or C-terminus to a string of histidine residues. Such fusion polypeptides can facilitate purification of recombinant NRP1, NID2, ENDO180, CDK10, FPGT, CARK, PCLO or REPS2. Expression vectors are commercially available that already encode a fusion moiety (e.g., a GST polypeptide), and a NRP1, NID2, ENDO180, CDK10, FPGT, CARK, PCLO or REPS2 nucleic acid can be cloned into an expression vector such that the fusion moiety is linked in-frame to the NRP1, NID2, ENDO180, CDK10, FPGT, CARK, PCLO or REPS2 polypeptide. Further, the fusion polypeptide can be a NRP1, NID2, ENDO180, CDK10, FPGT, CARK, PCLO or REPS2 polypeptide containing a heterologous signal sequence at its N-terminus. In certain host cells (e.g., mammalian host cells), expression, secretion, cellular internalization, and cellular localization of a NRP1, NID2, ENDO180, CDK10, FPGT, CARK, PCLO or REPS2 polypeptide can be increased through use of a heterologous signal sequence. Fusion polypeptides can also include all or a part of a serum polypeptide (e.g., an IgG constant region or human serum albumin).

NRP1, NID2, ENDO180, CDK10, FPGT, CARK, PCLO or REPS2 polypeptides or fragments thereof can be incorporated into pharmaceutical compositions and administered to a subject in vivo. Administration of these NRP1, NID2, ENDO180, CDK10, FPGT, CARK, PCLO or REPS2 polypeptides can be used to affect the bioavailability of a NRP1, NID2, ENDO180, CDK10, FPGT, CARK, PCLO or REPS2 substrate and may effectively increase NRP1, NID2, ENDO180, CDK10, FPGT, CARK, PCLO or REPS2 biological activity in a cell. NRP1, NID2, ENDO180, CDK10, FPGT, CARK, PCLO or REPS2 fusion polypeptides may be useful therapeutically for the treatment of disorders caused by, for example, (i) aberrant modification or mutation of a gene encoding a NRP1, NID2, ENDO180, CDK10, FPGT, CARK, PCLO or REPS2 polypeptide; (ii) mis-regulation of the NRP1, NID2, ENDO180, CDK10, FPGT, CARK, PCLO or REPS2 gene; and (iii) aberrant post-translational modification of a NRP1, NID2, ENDO180, CDK10, FPGT, CARK, PCLO or REPS2 polypeptide. Also, NRP1, NID2, ENDO180, CDK10, FPGT, CARK, PCLO or REPS2 polypeptides can be used as immunogens to produce anti-NRP1, NID2, ENDO180, CDK10, FPGT, CARK, PCLO or REPS2 antibodies in a subject, to purify NRP1, NID2, ENDO180, CDK10, FPGT, CARK, PCLO or REPS2 ligands or binding partners, and in screening assays to identify molecules which inhibit or enhance the interaction of NRP1, NID2, ENDO180, CDK10, FPGT, CARK, PCLO or REPS2 with a NRP1, NID2, ENDO180, CDK10, FPGT, CARK, PCLO or REPS2 substrate.

In addition, polypeptides can be chemically synthesized using techniques known in the art (See, e.g., Creighton, 1983 Proteins. New York, N.Y.: W. H. Freeman and Company; and Hunkapiller et al., (1984) Nature July 12-18; 310(5973): 105-11). For example, a relative short fragment can be synthesized by use of a peptide synthesizer. Furthermore, if desired, nonclassical amino acids or chemical amino acid analogs can be introduced as a substitution or addition into the fragment sequence. Nonclassical amino acids or chemical amino acid analogs include but are not limited to ornithine (hereinafter referred to as Z), diaminobutyric acid (hereinafter referred to as B), norleucine (hereinafter referred to as O), pyrylalanine, thienylalanine, naphthylalanine and phenylglycine. Other examples of non-naturally occurring amino acids and non-classical amino acid replacements are alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, beta-alanine*, L-alpha-amino butyric acid*, L-gamma-amino butyric acid*, L-alpha-amino isobutyric acid*, L-epsilon-amino caproic acid#, 7-amino heptanoic acid*, L-methionine sulfone*, L-norleucine*, L-norvaline*, p-nitro-L-phenylalanine*, L-hydroxyproline#, L-thioproline*, methyl derivatives of phenylalanine (Phe) such as 4-methyl-Phe*, pentamethyl-Phe*, L-Phe (4-amino)#, L-Tyr (methyl)*, L-Phe (4-isopropyl)*, L-Tic (1,2,3,4-tetrahydroisoquinoline-3-carboxyl acid)*, L-diaminopropionic acid, L-Phe (4-benzyl)*, 2,4-diaminobutyric acid, 4-aminobutyric acid (gamma-Abu), 2-amino butyric acid (alpha-Abu), 6-amino hexanoic acid (epsilon-Ahx), 2-amino isobutyric acid (Aib), 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, fluoroamino acids, designer amino acids such as beta-methyl amino acids, Ca-methyl amino acids, Na-methyl amino acids, naphthyl alanine, amino acid analogs in general and the like. The notation * indicates a derivative having hydrophobic characteristics, # indicates a derivative having hydrophilic characteristics, and #* indicates a derivative having amphipathic characteristics.

Polypeptides and polypeptide fragments sometimes are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4; acetylation, formylation, oxidation, reduction; metabolic synthesis in the presence of tunicamycin; and the like. Additional post-translational modifications include, for example, N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The polypeptide fragments may also be modified with a detectable label, such as an enzymatic, fluorescent, isotopic or affinity label to allow for detection and isolation of the polypeptide.

Also provided are chemically modified derivatives of polypeptides that can provide additional advantages such as increased solubility, stability and circulating time of the polypeptide, or decreased immunogenicity (see e.g., U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the molecular weight often utilized is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polymers should be attached to the polypeptide with consideration of effects on functional or antigenic domains of the polypeptide. There are a number of attachment methods available to those skilled in the art (e.g., EP 0 401 384 (coupling PEG to G-CSF) and Malik et al. (1992) Exp Hematol. September; 20(8): 1028-35 (pegylation of GM-CSF using tresyl chloride)). For example, polyethylene glycol may be covalently bound through amino acid residues via a reactive group, such as a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues, glutamic acid residues and the C-terminal amino acid residue. Sulfhydryl groups may also be used as a reactive group for attaching the polyethylene glycol molecules. For therapeutic purposes, the attachment sometimes is at an amino group, such as attachment at the N-terminus or lysine group.

Proteins can be chemically modified at the N-terminus. Using polyethylene glycol as an illustration of such a composition, one may select from a variety of polyethylene glycol molecules (by molecular weight, branching, etc.), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus may be accomplished by reductive alkylation, which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

Substantially Identical NRP1, NID2. ENDO180. CDK10. FPGT. CARK, PCLO or REPS2 Nucleic Acids and Polypeptides

Nucleotide sequences and polypeptide sequences that are substantially identical to the nucleotide sequence of SEQ ID NO: 1-17 and the polypeptide sequences of SEQ ID NO: 18-27 are included herein. The term “substantially identical” as used herein refers to two or more nucleic acids or polypeptides sharing one or more identical nucleotide sequences or polypeptide sequences, respectively. Included are nucleotide sequences or polypeptide sequences that are 55%, 60%, 65%, 70%, 75%, 80%, 85%, 90%, 95% or more (each often within a 1%, 2%, 3% or 4% variability) identical to the NRP1, NID2, ENDO180, CDK10, FPGT, CARK, PCLO or REPS2 nucleotide sequence in SEQ ID NO: 1-17 or the NRP1, NID2, ENDO180, CDK10, FPGT, CARK, PCLO or REPS2 polypeptide sequences of SEQ ID NO: 18-27. One test for determining whether two nucleic acids are substantially identical is to determine the percent of identical nucleotide sequences or polypeptide sequences shared between the nucleic acids or polypeptides.

Calculations of sequence identity are often performed as follows. Sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is sometimes 30% or more, 40% or more, 50% or more, often 60% or more, and more often 70%, 80%, 90%, 100% of the length of the reference sequence. The nucleotides or amino acids at corresponding nucleotide or polypeptide positions, respectively, are then compared among the two sequences. When a position in the first sequence is occupied by the same nucleotide or amino acid as the corresponding position in the second sequence, the nucleotides or amino acids are deemed to be identical at that position. The percent identity between the two sequences is a function of the number of identical positions shared by the sequences, taking into account the number of gaps, and the length of each gap, introduced for optimal alignment of the two sequences.

Comparison of sequences and determination of percent identity between two sequences can be accomplished using a mathematical algorithm. Percent identity between two amino acid or nucleotide sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Also, percent identity between two amino acid sequences can be determined using the Needleman & Wunsch, J. Mol. Biol. 48: 444-453 (1970) algorithm which has been incorporated into the GAP program in the GCG software package (available at the http address www.gcg.com), using either a Blossum 62 matrix or a PAM250 matrix, and a gap weight of 16, 14, 12, 10, 8, 6, or 4 and a length weight of 1, 2, 3, 4, 5, or 6. Percent identity between two nucleotide sequences can be determined using the GAP program in the GCG software package (available at http address www.gcg.com), using a NWSgapdna.CMP matrix and a gap weight of 40, 50, 60, 70, or 80 and a length weight of 1, 2, 3, 4, 5, or 6. A set of parameters often used is a Blossum 62 scoring matrix with a gap open penalty of 12, a gap extend penalty of 4, and a frameshift gap penalty of 5.

Another manner for determining if two nucleic acids are substantially identical is to assess whether a polynucleotide homologous to one nucleic acid will hybridize to the other nucleic acid under stringent conditions. As use herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1989). Aqueous and non-aqueous methods are described in that reference and either can be used. An example of stringent hybridization conditions is hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C.

An example of a substantially identical nucleotide sequence to SEQ ID NO: 1-17 is one that has a different nucleotide sequence and still encodes a polypeptide sequence set forth in SEQ ID NO: 18-25. Another example is a nucleotide sequence that encodes a polypeptide having a polypeptide sequence that is more than 70% identical to, sometimes more than 75%, 80%, or 85% identical to, and often more than 90% and 95% or more identical to the polypeptide sequences set forth in SEQ ID NO: 18-25.

NRP1, NID2, ENDO180, CDK10, FPGT, CARK, PCLO or REPS2 nucleotide sequences and polypeptide sequences can be used as “query sequences” to perform a search against public databases to identify other family members or related sequences, for example. Such searches can be performed using the NBLAST and XBLAST programs (version 2.0) of Altschul et al., J. Mol. Biol. 215: 403-10 (1990). BLAST nucleotide searches can be performed with the NBLAST program, score=100, wordlength=12 to obtain nucleotide sequences homologous to NRP1, NID2, ENDO180, CDK10, FPGT, CARK, PCLO or REPS2 nucleic acid molecules. BLAST polypeptide searches can be performed with the XBLAST program, score=50, wordlength=3 to obtain amino acid sequences homologous to NRP1, NID2, ENDO180, CDK10, FPGT, CARK, PCLO or REPS2 polypeptides. To obtain gapped alignments for comparison purposes, Gapped BLAST can be utilized as described in Altschul et al., Nucleic Acids Res. 25(17): 3389-3402 (1997). When utilizing BLAST and Gapped BLAST programs, default parameters of the respective programs (e.g., XBLAST and NBLAST) can be used (see the http address www.ncbi.nlm.nih.gov).

A nucleic acid that is substantially identical to the nucleotide sequence of SEQ ID NO: 1-17 may include polymorphic sites at positions equivalent to those described herein (e.g., position 32137 in SEQ ID NO: 1) when the sequences are aligned. For example, using the alignment procedures described herein, SNPs in a sequence substantially identical to the sequence of SEQ ID NO: 1-17 can be identified at nucleotide positions that match (i.e., align) with nucleotides at SNP positions in SEQ ID NO: 1-17. Also, where a polymorphic variation is an insertion or deletion, insertion or deletion of a nucleotide sequence from a reference sequence can change the relative positions of other polymorphic sites in the nucleotide sequence.

Substantially identical NRP1, NID2, ENDO180, CDK10, FPGT, CARK, PCLO or REPS2 nucleotide and polypeptide sequences include those that are naturally occurring, such as allelic variants (same locus), splice variants, homologs (different locus), and orthologs (different organism) or can be non-naturally occurring. Non-naturally occurring variants can be generated by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product). Orthologs, homologs, allelic variants, and splice variants can be identified using methods known in the art. These variants normally comprise a nucleotide sequence encoding a polypeptide that is 50%, about 55% or more, often about 70-75% or more, more often about 80-85% or more, and typically about 90-95% or more identical to the amino acid sequences shown in SEQ ID NO: 18-27 or a fragment thereof. Such nucleic acid molecules can readily be identified as being able to hybridize under stringent conditions to the nucleotide sequence shown in SEQ ID NO: 1-17 or a fragment of this sequence. Nucleic acid molecules corresponding to orthologs, homologs, and allelic variants of the NRP1, NID2, ENDO180, CDK10, FPGT, CARK, PCLO or REPS2 nucleotide sequence can further be identified by mapping the sequence to the same chromosome or locus as the NRP1, NID2, ENDO180, CDK10, FPGT, CARK, PCLO or REPS2 nucleotide sequence or variant.

Also, substantially identical NRP1, NID2, ENDO180, CDK10, FPGT, CARK, PCLO or REPS2 nucleotide sequences may include codons that are altered with respect to the naturally occurring sequence for enhancing expression of a NRP1, NID2 ENDO180, CDK10, FPGT, CARK, PCLO or REPS2 polypeptide or polypeptide variant in a particular expression system. For example, the nucleic acid can be one in which one or more codons are altered, and often 10% or more or 20% or more of the codons are altered for optimized expression in bacteria (e.g., E. coli.), yeast (e.g., S. cervesiae), human (e.g., 293 cells), insect, or rodent (e.g., hamster) cells.

Methods for Identifying Subjects at Risk of Melanoma and Risk of Melanoma

Methods for determining whether a subject is at risk of melanoma are provided herein. These methods include detecting the presence or absence of one or more polymorphic variations associated with melanoma in a NRP1, NID2, ENDO180, CDK10, FPGT, CARK, PCLO or REPS2 nucleotide sequence, or substantially identical sequence thereof, in a sample from a subject, where the presence of such a polymorphic variation is indicative of the subject being at risk of melanoma. These genetic tests are useful for prognosing and/or diagnosing melanoma and often are useful for determining whether an individual is at an increased, intermediate or decreased risk of developing or having melanoma.

Thus, featured herein is a method for identifying a subject at risk of melanoma, which comprises detecting in a nucleic acid sample from the subject the presence or absence of a polymorphic variation associated with melanoma at a polymorphic site in a NRP1, NID2, ENDO180, CDK10, FPGT, CARK, PCLO or REPS2 nucleotide sequence. The nucleotide sequence often is selected from the group consisting of: (a) a nucleotide sequence of SEQ ID NO: 1-17; (b) a nucleotide sequence which encodes a polypeptide consisting of the amino acid sequence encoded by the nucleotide sequence of SEQ ID NO: 1-17; (c) a nucleotide sequence which encodes a polypeptide that is 90% or more identical to the amino acid sequence encoded by the nucleotide sequence of SEQ ID NO: 1-17; and (d) a fragment of a nucleotide sequence of (a), (b), or (c), where the fragment often comprises a polymorphic site; whereby the presence of the polymorphic variation is indicative of the subject being at risk of melanoma. A polymorphic variation assayed in the genetic test often is located in an intron, sometimes in a region surrounding the NRP1, NID2, ENDO180, CDK10, FPGT, CARK, PCLO or REPS2 open reading frame (e.g., within 50 kilobases (kb), 40 kb, 30 kb, 20 kb, 15, kb, 10 kb, 5 kb, 4 kb, 3 kb, 2 kb, or 1 kb of the open reading frame initiation site or termination site), and sometimes in an exon. Sometimes the polymorphic variation is not located in an exon (e.g., it sometimes is located in an intron or region upstream or downstream of a terminal intron or exon). As used herein, “SEQ ID NO: 1-17” refers to individual sequences in SEQ ID NO: 1, 2, 3, 4, 5, 6, 7, 8, 9, 10, 11, 12, 13, 14, 15, 16 or 17, each sequence being separately applicable to embodiments described herein.

Results from such genetic tests may be combined with other test results to diagnose melanoma. For example, genetic test results may be gathered, a patient sample may be ordered based on a determined predisposition to melanoma (e.g., a skin biopsy), the patient sample is analyzed, and the results of the analysis may be utilized to diagnose melanoma. Also, melanoma diagnostic tests are generated by stratifying populations into subpopulations having different progressions of melanoma and detecting polymorphic variations associated with different progressions of the melanoma, as described in further detail hereafter. In another embodiment, genetic test results are gathered, a patient's risk factors for developing melanoma are analyzed (e.g., exposure to sun and skin pigmentation), and a patient sample may be ordered based on a determined risk of melanoma.

Risk of melanoma sometimes is expressed as a probability, such as an odds ratio, percentage, or risk factor. The risk assessment is based upon the presence or absence of one or more polymorphic variants described herein, and also may be based in part upon phenotypic traits of the individual being tested. Methods for calculating risks based upon patient data are well known (see, e.g., Agresti, Categorical Data Analysis, 2nd Ed. 2002. Wiley). Allelotyping and genotyping analyses may be carried out in populations other than those exemplified herein to enhance the predictive power of the prognostic method. These further analyses are executed in view of the exemplified procedures described herein, and may be based upon the same polymorphic variations or additional polymorphic variations.

The nucleic acid sample typically is isolated from a biological sample obtained from a subject. For example, nucleic acid can be isolated from blood, saliva, sputum, urine, cell scrapings, and biopsy tissue. The nucleic acid sample can be isolated from a biological sample using standard techniques, such as the technique described in Example 2. As used herein, the term “subject” refers primarily to humans but also refers to other mammals such as dogs, cats, and ungulates (e.g., cattle, sheep, and swine). Subjects also include avians (e.g., chickens and turkeys), reptiles, and fish (e.g., salmon), as embodiments described herein can be adapted to nucleic acid samples isolated from any of these organisms. The nucleic acid sample may be isolated from the subject and then directly utilized in a method for determining the presence of a polymorphic variant, or alternatively, the sample may be isolated and then stored (e.g., frozen) for a period of time before being subjected to analysis.

The presence or absence of a polymorphic variant is determined using one or both chromosomal complements represented in the nucleic acid sample. Determining the presence or absence of a polymorphic variant in both chromosomal complements represented in a nucleic acid sample from a subject having a copy of each chromosome is useful for determining the zygosity of an individual for the polymorphic variant (i.e., whether the individual is homozygous or heterozygous for the polymorphic variant). Any oligonucleotide-based diagnostic may be utilized to determine whether a sample includes the presence or absence of a polymorphic variant in a sample. For example, primer extension methods, ligase sequence determination methods (e.g., U.S. Pat. Nos. 5,679,524 and 5,952,174, and WO 01/27326), mismatch sequence determination methods (e.g., U.S. Pat. Nos. 5,851,770; 5,958,692; 6,110,684; and 6,183,958), microarray sequence determination methods, restriction fragment length polymorphism (RFLP), single strand conformation polymorphism detection (SSCP) (e.g., U.S. Pat. Nos. 5,891,625 and 6,013,499), PCR-based assays (e.g., TAQMAN® PCR System (Applied Biosystems)), and nucleotide sequencing methods may be used.

Oligonucleotide extension methods typically involve providing a pair of oligonucleotide primers in a polymerase chain reaction (PCR) or in other nucleic acid amplification methods for the purpose of amplifying a region from the nucleic acid sample that comprises the polymorphic variation. One oligonucleotide primer is complementary to a region 3′ of the polymorphism and the other is complementary to a region 5′ of the polymorphism. A PCR primer pair may be used in methods disclosed in U.S. Pat. Nos. 4,683,195; 4,683,202, 4,965,188; 5,656,493; 5,998,143; 6,140,054; WO 01/27327; and WO 01/27329 for example. PCR primer pairs may also be used in any commercially available machines that perform PCR, such as any of the GENEAMP® Systems available from Applied Biosystems. Also, those of ordinary skill in the art will be able to design oligonucleotide primers based upon a nucleotide sequence of SEQ ID NO: 1-17 without undue experimentation using knowledge readily available in the art.

Also provided are extension oligonucleotides that hybridize to the amplified fragment adjacent to the polymorphic variation. As used herein, the term “adjacent” refers to the 3′ end of the extension oligonucleotide being sometimes 1 nucleotide from the 5′ end of the polymorphic site, often 2 or 3, and at times 4, 5, 6, 7, 8, 9, or 10 nucleotides from the 5′ end of the polymorphic site, in the nucleic acid when the extension oligonucleotide is hybridized to the nucleic acid. The extension oligonucleotide then is extended by one or more nucleotides, often 2 or 3 nucleotides, and the number and/or type of nucleotides that are added to the extension oligonucleotide determine whether the polymorphic variant is present. Oligonucleotide extension methods are disclosed, for example, in U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; and WO 01/20039. Oligonucleotide extension methods using mass spectrometry are described, for example, in U.S. Pat. Nos. 5,547,835; 5,605,798; 5,691,141; 5,849,542; 5,869,242; 5,928,906; 6,043,031; and 6,194,144, and a method often utilized is described herein in Example 2.

A microarray can be utilized for determining whether a polymorphic variant is present or absent in a nucleic acid sample. A microarray sometimes includes an oligonucleotides described herein, and methods for making and using oligonucleotide microarrays suitable for prognostic use are disclosed in U.S. Pat. Nos. 5,492,806; 5,525,464; 5,589,330; 5,695,940; 5,849,483; 6,018,041; 6,045,996; 6,136,541; 6,142,681; 6,156,501; 6,197,506; 6,223,127; 6,225,625; 6,229,911; 6,239,273; WO 00/52625; WO 01/25485; and WO 01/29259. The microarray typically comprises a solid support and the oligonucleotides sometimes are linked to the solid support by covalent or non-covalent interactions. The oligonucleotides sometimes are linked to the solid support directly or by a spacer molecule. A microarray sometimes comprise one or more oligonucleotides complementary to a portion of SEQ ID NO: 1-17, or complementary to a variant described herein.

A kit may also be utilized for determining whether a polymorphic variant is present or absent in a nucleic acid sample. A kit often comprises one or more pairs of oligonucleotide primers useful for amplifying a fragment of SEQ ID NO: 1-17 or a substantially identical sequence thereof, where the fragment includes a polymorphic site. The kit sometimes comprises a polymerizing agent, for example, a thermostable nucleic acid polymerase such as one disclosed in U.S. Pat. No. 4,889,818 or 6,077,664. Also, the kit often comprises an elongation oligonucleotide that hybridizes to a NRP1, NID2, ENDO180, CDK180, FPGT, CARK, PCLO or REPS2 nucleic acid in a nucleic acid sample adjacent to the polymorphic site. Where the kit includes an elongation oligonucleotide, it also often comprises chain elongating nucleotides, such as dATP, dTTP, dGTP, dCTP, and dITP, including analogs of dATP, dTTP, dGTP, dCTP and dITP, provided that such analogs are substrates for a thermostable nucleic acid polymerase and can be incorporated into a nucleic acid chain elongated from the extension oligonucleotide. Along with chain elongating nucleotides would be one or more chain terminating nucleotides such as ddATP, ddTTP, ddGTP, ddCTP, and the like. In an embodiment, the kit comprises one or more oligonucleotide primer pairs, a polymerizing agent, chain elongating nucleotides, at least one elongation oligonucleotide, and one or more chain terminating nucleotides. Kits optionally include buffers, vials, microtitre plates, and instructions for use. NRP1, NID2, ENDO180, CDK10, FPGT, CARK, PCLO or REPS2 directed hits may be utilized to prognose or diagnose melanoma for a significant fraction of melanoma occurrences, such as in 50% or more melanoma occurrences, or sometimes 60% or more, 70% or more, 80% or more, 90% or more, or 95% or more melanoma occurrences.

Using a polymorphism detection technology (e.g., a technique described above or below in Example 2), mutations and polymorphisms in or around the NRP1, NID2, ENDO180, CDK10, FPGT, CARK, PCLO or REPS2 locus may be detected in melanocytic lesions, which include nevi, radial growth phase (RGP) melanomas, vertical growth phase (VGP) melanomas, and melanoma metastases. The mutations can be detected within 50 kilobases (kb), 40 kb, 30 kb, 20 kb, 15, kb, 10 kb, 5 kb, 4 kb, 3 kb, 2 kb, or 1 kb from the NRP1, NID2, ENDO180, CDK10, FPGT, CARK, PCLO or REPS2 open reading frame initiation or termination site. Therefore, provided herein are methods for genotyping NRP1, NID2, ENDO180, CDK10, FPGT, CARK, PCLO or REPS2 mutations in melanocytic lesions and metastases (e.g., described in Example 2). Mutations in or around the NRP1, NID2, ENDO180, CDK10, FPGT, CARK, PCLO or REPS2 loci present in later stage melanomas, such as VGP melanomas and melanoma metastases, are indicative of melanomas particularly likely to continue to progress and/or metastasize (e.g., from RGP to VGP melanoma or melanoma metastases), i.e., aggressive melanomas. Thus, provided herein are methods for identifying subjects at risk of a progressive or aggressive melanoma by determining the presence or absence of one or more NRP1, NID2, ENDO180, CDK10, FPGT, CARK, PCLO or REPS2 mutations in the DNA sample of a subject that exist in melanocytic lesions and/or metastases. Identifying the presence of one or more of these mutations is useful for identifying subjects in need of aggressive treatments of melanoma, and once identified using such methods, a subject often is given information concerning preventions and treatments of the disease, and sometimes is treated with an aggressive melanoma treatment method (e.g., surgery or administration of drugs), as described in more detail hereafter.

Determining the presence of a polymorphic variant, or a combination of two or more polymorphic variants, in a nucleic acid set forth in SEQ ID NO: 1-17, or SEQ ID NO: 1-7, of the sample often is indicative of a predisposition to melanoma. In certain embodiments, nucleic acid variants of other loci, such as the BRAF locus described in U.S. application Ser. No. 10/661,966 filed Sep. 11, 2003 for example, are detected in combination with one or more nucleic acid variants in the NRP1, NID2, ENDO180, CDK10, FPGT, CARK, PCLO or REPS2 loci.

The following variants are associated with an increased risk of melanoma: a thymine at the chromosome position indicated by rs1360457, an adenine at the chromosome position indicated by rs753050, a guanine at the chromosome position indicated by rs729647, a thymine at the chromosome position indicated by rs3742536, a guanine at position 32137 in SEQ ID NO: 1, a thymine at position 12008 in SEQ ID NO: 1, a cytosine at position 32720 in SEQ ID NO: 1, an adenine at position 43721 in SEQ ID NO: 1, a guanine at position 44339 in SEQ ID NO: 1, a thymine at position 45640 in SEQ ID NO: 1, an adenine at position 48768 in SEQ ID NO: 1, a guanine at position 74247 in SEQ ID NO: 1, a thymine at position 75828 in SEQ ID NO: 1, an adenine at position 76381 in SEQ ID NO: 1, and a cytosine at position 84909. An individual identified as being at risk of melanoma may be heterozygous or homozygous with respect to the allele associated with melanoma. A subject homozygous for an allele associated with an increased risk of melanoma (e.g., a thymine at position 45640 in SEQ ID NO: 1) is at a comparatively high risk of melanoma, a subject heterozygous for an allele associated with an increased risk of melanoma is at a comparatively intermediate risk of melanoma, and a subject homozygous for an allele associated with a decreased risk of melanoma (e.g., a cytosine at position 45640 in SEQ ID NO: 1, see Examples section below) is at a comparatively lower risk of melanoma. A genotype may be assessed for a complementary strand, such that the complementary nucleotide at a particular position is detected (e.g., if a thymine or cytosine is detected at position 45640 in SEQ ID NO: 1, the complementary strand would yield an adenine or guanine, respectively, where the adenine is associated with increased risk of melanoma).

Also featured are methods for determining risk of melanoma and/or identifying a subject at risk of melanoma by contacting a NRP1, NID2, ENDO180, CDK10, FPGT, CARK, PCLO or REPS2 polypeptide or protein from a subject with an antibody that specifically binds to an epitope associated with increased risk of melanoma in the polypeptide. In an embodiment, the antibody specifically binds to an epitope that comprises a serine at amino acid position 656 in a NID2 polypeptide.

Applications of Genomic Information to Pharmacogenomics

Pharmacogenomics is a discipline that involves tailoring a treatment for a subject according to the subject's genotype as a particular treatment regimen may exert a differential effect depending upon the subject's genotype. Based upon the outcome of a prognostic test described herein, a clinician or physician may target pertinent information and preventative or therapeutic treatments to a subject who would be benefited by the information or treatment and avoid directing such information and treatments to a subject who would not be benefited (e.g., the treatment has no therapeutic effect and/or the subject experiences adverse side effects).

For example, where a candidate therapeutic exhibits a significant interaction with a major allele and a comparatively weak interaction with a minor allele (e.g., an order of magnitude or greater difference in the interaction), such a therapeutic typically would not be administered to a subject genotyped as being homozygous for the minor allele, and sometimes not administered to a subject genotyped as being heterozygous for the minor allele. In another example, where a candidate therapeutic is not significantly toxic when administered to subjects who are homozygous for a major allele but is comparatively toxic when administered to subjects heterozygous or homozygous for a minor allele, the candidate therapeutic is not typically administered to subjects who are genotyped as being heterozygous or homozygous with respect to the minor allele.

The prognostic methods described herein are applicable to general pharmacogenomic approaches towards addressing melanoma. For example, a nucleic acid sample from an individual may be subjected to a prognostic test described herein. Where one or more polymorphic variations associated with increased risk of melanoma are identified in a subject, one or more melanoma treatments or prophylactic regimens may be prescribed to that subject. Subjects genotyped as having one or more of the alleles described herein that are associated with increased risk of melanoma often are prescribed a prophylactic regimen designed to minimize the occurrence of melanoma. An example of a prophylactic regimen often prescribed is directed towards minimizing ultraviolet (UV) light exposure. Such a regimen may include, for example, prescription of a lotion applied to the skin that minimizes UV penetration and/or counseling individuals of other practices for reducing UV exposure, such as by wearing protective clothing and minimizing sun exposure.

In certain embodiments, a treatment regimen is specifically prescribed and/or administered to individuals who will most benefit from it based upon their risk of developing melanoma assessed by the prognostic methods described herein. Thus, provided are methods for identifying a subject predisposed to melanoma and then prescribing a therapeutic or preventative regimen to individuals identified as having a predisposition. Thus, certain embodiments are directed to a method for reducing melanoma in a subject, which comprises: detecting the presence or absence of a polymorphic variant associated with melanoma in a nucleotide sequence of a nucleic acid sample from a subject, where the nucleotide sequence comprises a polynucleotide sequence selected from the group consisting of: (a) a nucleotide sequence set forth in SEQ ID NO: 1-17; (b) a nucleotide sequence which encodes a polypeptide consisting of an amino acid sequence encoded by a nucleotide sequence of SEQ ID NO: 1-17; (c) a nucleotide sequence which encodes a polypeptide that is 90% or more identical to an amino acid sequence encoded by a nucleotide sequence of SEQ ID NO: 1-17 or a nucleotide sequence about 90% or more identical to the nucleotide sequence in SEQ ID NO: 1-17; and (d) a fragment of a polynucleotide sequence of (a), (b), or (c); and prescribing or administering a treatment regimen to a subject from whom the sample originated where the presence of a polymorphic variation associated with melanoma is detected in the nucleotide sequence. In these methods, predisposition results may be utilized in combination with other test results to diagnose melanoma.

The treatment sometimes is preventative (e.g., is prescribed or administered to reduce the probability that a melanoma associated condition arises or progresses), sometimes is therapeutic, and sometimes delays, alleviates or halts the progression of a melanoma associated condition. Any known preventative or therapeutic treatment for alleviating or preventing the occurrence of a melanoma associated disorder is prescribed and/or administered. For example, the treatment sometimes is or includes a drug that reduces melanoma, including, for example, cisplatin, carmustine (BCNU), vinblastine, vincristine, and bleomycin, and/or a molecule that interacts with a nucleic acid or polypeptide described hereafter. In another example, the melanoma treatment is surgery. Surgery to remove (excise) a melanoma is the standard treatment for this disease. It is necessary to remove not only the tumor but also some normal tissue around it in order to minimize the chance that any cancer will be left in the area. It is common for lymph nodes near the tumor to be removed during surgery because cancer can spread through the lymphatic system. Surgery is generally not effective in controlling melanoma that is known to have spread to other parts of the body. In such cases, doctors may use other methods of treatment, such as chemotherapy, biological therapy, radiation therapy, or a combination of these methods.

As therapeutic approaches for melanoma continue to evolve and improve, the goal of treatments for melanoma related disorders is to intervene even before clinical signs (e.g., identification of irregular nevi based on A—asymmetry, B—border irregularity, C—color variation, D—diameter of >6 mm as described by Friedman R J, et al. in CA Cancer J. Clin. 1985 May-June; 35(3): 130-51) first manifest. Thus, genetic markers associated with susceptibility to melanoma prove useful for early diagnosis, prevention and treatment of melanoma.

As melanoma preventative and treatment information can be specifically targeted to subjects in need thereof (e.g., those at risk of developing melanoma or those that have early signs of melanoma), provided herein is a method for preventing or reducing the risk of developing melanoma in a subject, which comprises: (a) detecting the presence or absence of a polymorphic variation associated with melanoma at a polymorphic site in a nucleotide sequence in a nucleic acid sample from a subject; (b) identifying a subject with a predisposition to melanoma, whereby the presence of the polymorphic variation is indicative of a predisposition to melanoma in the subject; and (c) if such a predisposition is identified, providing the subject with information about methods or products to prevent or reduce melanoma or to delay the onset of melanoma. Also provided is a method of targeting information or advertising to a subpopulation of a human population based on the subpopulation being genetically predisposed to a disease or condition, which comprises: (a) detecting the presence or absence of a polymorphic variation associated with melanoma at a polymorphic site in a nucleotide sequence in a nucleic acid sample from a subject; (b) identifying the subpopulation of subjects in which the polymorphic variation is associated with melanoma; and (c) providing information only to the subpopulation of subjects about a particular product which may be obtained and consumed or applied by the subject to help prevent or delay onset of the disease or condition.

Pharmacogenomics methods also may be used to analyze and predict a response to a melanoma treatment or a drug. For example, if pharmacogenomics analysis indicates a likelihood that an individual will respond positively to a melanoma treatment with a particular drug, the drug may be administered to the individual. Conversely, if the analysis indicates that an individual is likely to respond negatively to treatment with a particular drug, an alternative course of treatment may be prescribed. A negative response may be defined as either the absence of an efficacious response or the presence of toxic side effects. The response to a therapeutic treatment can be predicted in a background study in which subjects in any of the following populations are genotyped: a population that responds favorably to a treatment regimen, a population that does not respond significantly to a treatment regimen, and a population that responds adversely to a treatment regiment (e.g., exhibits one or more side effects). These populations are provided as examples and other populations and subpopulations may be analyzed. Based upon the results of these analyses, a subject is genotyped to predict whether he or she will respond favorably to a treatment regimen, not respond significantly to a treatment regimen, or respond adversely to a treatment regimen.

The tests described herein also are applicable to clinical drug trials. One or more polymorphic variants indicative of response to an agent for treating melanoma or to side effects to an agent for treating melanoma may be identified using the methods described herein. Thereafter, potential participants in clinical trials of such an agent may be screened to identify those individuals most likely to respond favorably to the drug and exclude those likely to experience side effects. In that way, the effectiveness of drug treatment may be measured in individuals who respond positively to the drug, without lowering the measurement as a result of the inclusion of individuals who are unlikely to respond positively in the study and without risking undesirable safety problems.

Thus, another embodiment is a method of selecting an individual for inclusion in a clinical trial of a treatment or drug comprising the steps of: (a) obtaining a nucleic acid sample from an individual; (b) determining the identity of a polymorphic variation which is associated with a positive response to the treatment or the drug, or at least one polymorphic variation which is associated with a negative response to the treatment or the drug in the nucleic acid sample, and (c) including the individual in the clinical trial if the nucleic acid sample contains said polymorphic variation associated with a positive response to the treatment or the drug or if the nucleic acid sample lacks said polymorphic variation associated with a negative response to the treatment or the drug. In addition, the methods for selecting an individual for inclusion in a clinical trial of a treatment or drug encompass methods with any further limitation described in this disclosure, or those following, specified alone or in any combination. The polymorphic variation may be in a sequence selected individually or in any combination from the group consisting of (i) a polynucleotide sequence set forth in SEQ ID NO: 1-17; (ii) a polynucleotide sequence that is 90% or more identical to an amino acid sequence encoded by a nucleotide sequence of SEQ ID NO: 1-17; (iii) a polynucleotide sequence that encodes a polypeptide having an amino acid sequence identical to or 90% or more identical to an amino acid sequence encoded by a nucleotide sequence of SEQ ID NO: 1-17; and (iv) a fragment of a polynucleotide sequence of (i), (ii), or (iii) comprising the polymorphic site. The including step (c) optionally comprises administering the drug or the treatment to the individual if the nucleic acid sample contains the polymorphic variation associated with a positive response to the treatment or the drug and the nucleic acid sample lacks said biallelic marker associated with a negative response to the treatment or the drug.

Also provided herein is a method of partnering between a diagnostic/prognostic testing provider and a provider of a consumable product, which comprises: (a) the diagnostic/prognostic testing provider detects the presence or absence of a polymorphic variation associated with melanoma at a polymorphic site in a nucleotide sequence in a nucleic acid sample from a subject; (b) the diagnostic/prognostic testing provider identifies the subpopulation of subjects in which the polymorphic variation is associated with melanoma; (c) the diagnostic/prognostic testing provider forwards information to the subpopulation of subjects about a particular product which may be obtained and consumed or applied by the subject to help prevent or delay onset of the disease or condition; and (d) the provider of a consumable product forwards to the diagnostic test provider a fee every time the diagnostic/prognostic test provider forwards information to the subject as set forth in step (c) above.

Compositions Comprising a NRP1. NID2. ENDO180. CDK10. FPGT. CARK. PCLO or REPS2 Directed Molecule

Featured herein is a composition comprising a melanoma cell and one or more NRP1, NID2, ENDO180, CDK10, FPGT, CARK, PCLO or REPS2 directed molecules. NRP1, NID2, ENDO180, CDK10, FPGT, CARK, PCLO or REPS2 directed molecules include, but are not limited to, a compound that binds to a NRP1, NID2, ENDO180, CDK10, FPGT, CARK, PCLO or REPS2 nucleic acid or polypeptide; an RNAi or siRNA molecule having a strand complementary to a NRP1, NID2, ENDO180, CDK10, FPGT, CARK, PCLO or REPS2 DNA sequence; an antisense nucleic acid complementary to an RNA encoded by a NRP1, NID2, ENDO180, CDK10, FPGT, CARK, PCLO or REPS2 DNA sequence; a nucleic acid complementary to a NRP1, NID2, ENDO180, CDK10, FPGT, CARK, PCLO or REPS2 RNA, cDNA or gDNA nucleotide sequence, sometimes linked to a solid support or an array; a ribozyme that hybridizes to a NRP1, NID2, ENDO180, CDK10, FPGT, CARK, PCLO or REPS2 nucleotide sequence; an NRP1, NID2, ENDO180, CDK10, FPGT, CARK, PCLO or REPS2 polypeptide, protein or fragment thereof, or a nucleic acid that encodes the foregoing; a nucleic acid aptamer that specifically binds a NRP1, NID2, ENDO180, CDK10, FPGT, CARK, PCLO or REPS2 polypeptide, protein, nucleic acid or variant thereof; and an antibody or fragment thereof that specifically binds to a NRP1, NID2, ENDO180, CDK10, FPGT, CARK, PCLO or REPS2 polypeptide, protein, nucleic acid or variant thereof. Compositions comprising an antibody often include an adjuvant known in the art. The melanoma cell may be in a group of melanoma cells and/or other types of cells cultured in vitro or in a tissue having melanoma cells (e.g., a melanocytic lesion) maintained in vitro or present in an animal in vivo (e.g., a rat, mouse, ape or human). In certain embodiments, a composition comprises a component from a melanoma cell or from a subject having a melanoma cell instead of the melanoma cell or in addition to the melanoma cell, where the component sometimes is a nucleic acid molecule (e.g., genomic DNA), a protein mixture or isolated protein, for example. The aforementioned compositions have utility in diagnostic, prognostic and pharmacogenomic methods described previously and in melanoma therapeutics described hereafter. Certain NRP1, NID2, ENDO180, CDK10, FPGT, CARK, PCLO or REPS2 directed molecules are described in greater detail below.

Compounds

Compounds can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive (see, e.g., Zuckermann et al., J. Med. Chem. 37: 2678-85 (1994)); spatially addressable parallel solid phase or solution phase libraries; synthetic library methods requiring deconvolution; “one-bead one-compound” library methods; and synthetic library methods using affinity chromatography selection. Biological library and peptoid library approaches are typically limited to peptide libraries, while the other approaches are applicable to peptide, non-peptide oligomer or small molecule libraries of compounds (Lam, Anticancer Drug Des. 12: 145, (1997)). Examples of methods for synthesizing molecular libraries are described, for example, in DeWitt et al., Proc. Natl. Acad. Sci. USA. 90: 6909 (1993); Erb et al., Proc. Natl. Acad. Sci. USA 91: 11422 (1994); Zuckermann et al., J. Med. Chem. 37: 2678 (1994); Cho et al., Science 261: 1303 (1993); Carrell et al., Angew. Chem. Int. Ed. Engl. 33: 2059 (1994); Carell et al., Angew. Chem. Int. Ed. Engl. 33: 2061 (1994); and in Gallop et al., J. Med. Chem. 37: 1233 (1994).

Libraries of compounds may be presented in solution (e.g., Houghten, Biotechniques 13: 412421 (1992)), or on beads (Lam, Nature 354: 82-84 (1991)), chips (Fodor, Nature 364: 555-556 (1993)), bacteria or spores (Ladner, U.S. Pat. No. 5,223,409), plasmids (Cull et al., Proc. Natl. Acad. Sci. USA 89: 1865-1869 (1992)) or on phage (Scott and Smith, Science 249: 386-390 (1990); Devlin, Science 249: 404-406 (1990); Cwirla et al., Proc. Natl. Acad. Sci. 87: 6378-6382 (1990); Felici, J. Mol. Biol. 222: 301-310 (1991); Ladner supra.).

A compound may alter expression or activity of NRP1, NID2, ENDO180, CDK10, FPGT, CARK, PCLO or REPS2 polypeptides and may be a small molecule. Small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Antisense Nucleic Acid Molecules, Ribozymes, RNAi, siRNA and Modified NRP1, NID2. ENDO180. CDK10. FPGT, CARK, PCLO or REPS2 Nucleic Acid Molecules

An “antisense” nucleic acid refers to a nucleotide sequence complementary to a “sense” nucleic acid encoding a polypeptide, e.g., complementary to the coding strand of a double-stranded cDNA molecule or complementary to an mRNA sequence. The antisense nucleic acid can be complementary to an entire NRP1, NID2, ENDO180, CDK10, FPGT, CARK, PCLO or REPS2 coding strand, or to only a portion thereof (e.g., coding regions of human NRP1, NID2, ENDO180, CDK10, FPGT, CARK, PCLO or REPS2 in SEQ ID NO: 1-17). In another embodiment, the antisense nucleic acid molecule is antisense to a “noncoding region” of the coding strand of a nucleotide sequence encoding NRP1, NID2, ENDO180, CDK10, FPGT, CARK, PCLO or REPS2 (e.g., 5′ and 3′ untranslated regions).

An antisense nucleic acid can be designed such that it is complementary to the entire coding region of NRP1, NID2, ENDO180, CDK10, FPGT, CARK, PCLO or REPS2 mRNA, and often the antisense nucleic acid is an oligonucleotide antisense to only a portion of a coding or noncoding region of NRP1, NID2, ENDO180, CDK10, FPGT, CARK, PCLO or REPS2 mRNA. For example, the antisense oligonucleotide can be complementary to the region surrounding the translation start site of NRP1, NID2, ENDO180, CDK10, FPGT, CARK, PCLO or REPS2 mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length. The antisense nucleic acids, which include the ribozymes described hereafter, can be designed to target NRP1, NID2, ENDO180, CDK10, FPGT, CARK, PCLO or REPS2 nucleic acid or NRP1, NID2, ENDO180, CDK10, FPGT, CARK, PCLO or REPS2 nucleic acid variants. Among the variants, minor alleles and major alleles can be targeted, and those associated with a higher risk of melanoma are often designed, tested, and administered to subjects.

An antisense nucleic acid can be constructed using chemical synthesis and enzymatic ligation reactions using standard procedures. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

When utilized as therapeutics, antisense nucleic acids typically are administered to a subject (e.g., by direct injection at a tissue site) or generated in situ such that they hybridize with or bind to cellular mRNA and/or genomic DNA encoding a NRP1, NID2, ENDO180, CDK10, FPGT, CARK, PCLO or REPS2 polypeptide and thereby inhibit expression of the polypeptide, for example, by inhibiting transcription and/or translation. Alternatively, antisense nucleic acid molecules can be modified to target selected cells and then are administered systemically. For systemic administration, antisense molecules can be modified such that they specifically bind to receptors or antigens expressed on a selected cell surface, for example, by linking antisense nucleic acid molecules to peptides or antibodies which bind to cell surface receptors or antigens. Antisense nucleic acid molecules can also be delivered to cells using the vectors described herein. Sufficient intracellular concentrations of antisense molecules are achieved by incorporating a strong promoter, such as a pol II or pol III promoter, in the vector construct.

Antisense nucleic acid molecules sometimes are α-anomeric nucleic acid molecules. An α-anomeric nucleic acid molecule forms specific double-stranded hybrids with complementary RNA in which, contrary to the usual β-units, the strands run parallel to each other (Gaultier et al., Nucleic Acids. Res. 15: 6625-6641 (1987)). Antisense nucleic acid molecules can also comprise a 2′—O— methylribonucleotide (Inoue et al., Nucleic Acids Res. 15: 6131-6148 (1987)) or a chimeric RNA-DNA analogue (Inoue et al., FEBS Lett. 215: 327-330 (1987)). Antisense nucleic acids sometimes are composed of DNA or PNA or any other nucleic acid derivatives described previously.

In another embodiment, an antisense nucleic acid is a ribozyme. A ribozyme having specificity for a NRP1, NID2, ENDO180, CDK10, FPGT, CARK, PCLO or REPS2 encoding nucleic acid can include one or more sequences complementary to the nucleotide sequence of a NRP1, NID2, ENDO180, CDK10, FPGT, CARK, PCLO or REPS2 DNA sequence disclosed herein (e.g., SEQ ID NO: 1-17), and a sequence having a known catalytic region responsible for mRNA cleavage (see e.g., U.S. Pat. No. 5,093,246 or Haselhoff and Gerlach, Nature 334: 585-591 (1988)). For example, a derivative of a Tetrahymena L-19 IVS RNA is sometimes utilized in which the nucleotide sequence of the active site is complementary to the nucleotide sequence to be cleaved in a NRP1, NID2, ENDO180, CDK10, FPGT, CARK, PCLO or REPS2 encoding mRNA (see e.g., Cech et al. U.S. Pat. No. 4,987,071; and Cech et al. U.S. Pat. No. 5,116,742). Also, NRP1, NID2, ENDO180, CDK10, FPGT, CARK, PCLO or REPS2 mRNA can be used to select a catalytic RNA having a specific ribonuclease activity from a pool of RNA molecules (see e.g., Bartel & Szostak, Science 261: 1411-1418 (1993)).

NRP1, NID2, ENDO180, CDK10, FPGT, CARK, PCLO or REPS2 directed molecules include in certain embodiments nucleic acids that can form triple helix structures with a NRP1, NID2, ENDO180, CDK10, FPGT, CARK, PCLO or REPS2 nucleotide sequence, especially one that includes a regulatory region that controls NRP1, NID2, ENDO180, CDK10, FPGT, CARK, PCLO or REPS2 expression. NRP1, NID2, ENDO180, CDK10, FPGT, CARK, PCLO or REPS2 gene expression can be inhibited by targeting nucleotide sequences complementary to the regulatory region of the NRP1, NID2, ENDO180, CDK10, FPGT, CARK, PCLO or REPS2 (e.g., NRP1, NID2, ENDO80, CDK10, FPGT, CARK, PCLO or REPS2 promoter and/or enhancers) to form triple helical structures that prevent transcription of the NRP1, NID2, ENDO180, CDK10, FPGT, CARK, PCLO or REPS2 gene in target cells (see e.g., Helene, Anticancer Drug Des. 6(6): 569-84 (1991); Helene et al., Ann. N.Y. Acad. Sci. 660: 27-36 (1992); and Maher, Bioassays 14(12): 807-15 (1992). Potential sequences that can be targeted for triple helix formation can be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

NRP1, NID2, ENDO180, CDK10, FPGT, CARK, PCLO or REPS2 directed molecules include RNAi and siRNA nucleic acids. Gene expression may be inhibited by the introduction of double-stranded RNA (dsRNA), which induces potent and specific gene silencing, a phenomenon called RNA interference or RNAi. See, e.g., Fire et al., U.S. Pat. No. 6,506,559; Tuschl et al. PCT International Publication No. WO 01/75164; Kay et al. PCT International Publication No. WO 03/010180A1; or Bosher J M, Labouesse, Nat Cell Biol 2000 February; 2(2): E31-6. This process has been improved by decreasing the size of the double-stranded RNA to 20-24 base pairs (to create small-interfering RNAs or siRNAs) that “switched off” genes in mammalian cells without initiating an acute phase response, i.e., a host defense mechanism that often results in cell death (see, e.g., Caplen et al. Proc Natl Acad Sci USA. 2001 Aug. 14; 98(17): 9742-7 and Elbashir et al. Methods 2002 February; 26(2): 199-213). There is increasing evidence of post-transcriptional gene silencing by RNA interference (RNAi) for inhibiting targeted expression in mammalian cells at the mRNA level, in human cells. There is additional evidence of effective methods for inhibiting the proliferation and migration of tumor cells in human patients, and for inhibiting metastatic cancer development (see, e.g., U.S. patent application No. U.S.2001000993183; Caplen et al. Proc Natl Acad Sci USA; and Abderrahmani et al. Mol Cell Biol 2001 November 21(21): 7256-67).

An “siRNA” or “RNAi” refers to a nucleic acid that forms a double stranded RNA and has the ability to reduce or inhibit expression of a gene or target gene when the siRNA is delivered to or expressed in the same cell as the gene or target gene. “siRNA” refers to short double-stranded RNA formed by the complementary strands. Complementary portions of the siRNA that hybridize to form the double stranded molecule often have substantial or complete identity to the target molecule sequence. In one embodiment, an siRNA refers to a nucleic acid that has substantial or complete identity to a target gene and forms a double stranded siRNA, such as a nucleotide sequence set forth in SEQ ID NO: 1-17, for example.

When designing the siRNA molecules, the targeted region often is selected from a given DNA sequence beginning 50 to 100 nucleotides downstream of the start codon. See, e.g., Elbashir et al,. Methods 26: 199-213 (2002). Initially, 5′ or 3′ UTRs and regions nearby the start codon were avoided assuming that UTR-binding proteins and/or translation initiation complexes may interfere with binding of the siRNP or RISC endonuclease complex. Sometimes regions of the target 23 nucleotides in length conforming to the sequence motif AA(N19)TT (N, an nucleotide), and regions with approximately 30% to 70% G/C-content (often about 50% G/C-content) often are selected. If no suitable sequences are found, the search often is extended using the motif NA(N21). The sequence of the sense siRNA sometimes corresponds to (N19) TT or N21 (position 3 to 23 of the 23-nt motif), respectively. In the latter case, the 3′ end of the sense siRNA often is converted to TT. The rationale for this sequence conversion is to generate a symmetric duplex with respect to the sequence composition of the sense and antisense 3′ overhangs. The antisense siRNA is synthesized as the complement to position 1 to 21 of the 23-nt motif. Because position 1 of the 23-nt motif is not recognized sequence-specifically by the antisense siRNA, the 3′-most nucleotide residue of the antisense siRNA can be chosen deliberately. However, the penultimate nucleotide of the antisense siRNA (complementary to position 2 of the 23-nt motif) often is complementary to the targeted sequence. For simplifying chemical synthesis, TT often is utilized. siRNAs corresponding to the target motif NAR(N 17)YNN, where R is purine (A,G) and Y is pyrimidine (C,U), often are selected. Respective 21 nucleotide sense and antisense siRNAs often begin with a purine nucleotide and can also be expressed from pol III expression vectors without a change in targeting site. Expression of RNAs from pol III promoters often is efficient when the first transcribed nucleotide is a purine.

The sequence of the siRNA can correspond to the full length target gene, or a subsequence thereof. Often, the siRNA is about 15 to about 50 nucleotides in length (e.g., each complementary sequence of the double stranded siRNA is 15-50 nucleotides in length, and the double stranded siRNA is about 15-50 base pairs in length, sometimes about 20-30 nucleotides in length or about 20-25 nucleotides in length, e.g., 20, 21, 22, 23, 24, 25, 26, 27, 28, 29, or 30 nucleotides in length. The siRNA sometimes is about 21 nucleotides in length. Methods of using siRNA are well known in the art, and specific siRNA molecules may be purchased from a number of companies including Dharmacon Research, Inc.

Antisense, ribozyme, RNAi and siRNA nucleic acids can be altered to form modified NRP1, NID2 ENDO180, CDK10, FPGT, CARK, PCLO or REPS2 nucleic acid molecules. The nucleic acids can be altered at base moieties, sugar moieties or phosphate backbone moieties to improve stability, hybridization, or solubility of the molecule. For example, the deoxyribose phosphate backbone of nucleic acid molecules can be modified to generate peptide nucleic acids (see Hyrup et al., Bioorganic & Medicinal Chemistry 4 (1): 5-23 (1996)). As used herein, the terms “peptide nucleic acid” or “PNA” refers to a nucleic acid mimic such as a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. Synthesis of PNA oligomers can be performed using standard solid phase peptide synthesis protocols as described, for example, in Hyrup et al., (1996) supra and Perry-O'Keefe et al., Proc. Natl. Acad. Sci. 93: 14670-675 (1996).

PNAs of NRP1, NID2, ENDO180, CDK10, FPGT, CARK, PCLO or REPS2 nucleic acids can be used in prognostic, diagnostic, and therapeutic applications. For example, PNAs can be used as antisense or antigene agents for sequence-specific modulation of gene expression by, for example, inducing transcription or translation arrest or inhibiting replication. PNAs of NRP1, NID2, ENDO180, CDK10, FPGT, CARK, PCLO or REPS2 nucleic acid molecules can also be used in the analysis of single base pair mutations in a gene, (e.g., by PNA-directed PCR clamping); as “artificial restriction enzymes” when used in combination with other enzymes, (e.g., S1 nucleases (Hyrup (1996) supra)); or as probes or primers for DNA sequencing or hybridization (Hyrup et al., (1996) supra; Perry-O'Keefe supra).

In other embodiments, oligonucleotides may include other appended groups such as peptides (e.g., for targeting host cell receptors in vivo), or agents facilitating transport across cell membranes (see e.g., Letsinger et al., Proc. Natl. Acad. Sci. USA 86: 6553-6556 (1989); Lemaitre et al., Proc. Natl. Acad. Sci. USA 84: 648-652 (1987); PCT Publication No. WO88/09810) or the blood-brain barrier (see, e.g., PCT Publication No. WO89/10134). In addition, oligonucleotides can be modified with hybridization-triggered cleavage agents (See, e.g., Krol et al., Bio-Techniques 6: 958-976 (1988)) or intercalating agents. (See, e.g., Zon, Pharm. Res. 5: 539-549 (1988)). To this end, the oligonucleotide may be conjugated to another molecule, (e.g., a peptide, hybridization triggered cross-linking agent, transport agent, or hybridization-triggered cleavage agent).

Also included herein are molecular beacon oligonucleotide primer and probe molecules having one or more regions complementary to a NRP1, NID2, ENDO180, CDK10, FPGT, CARK, PCLO or REPS2 nucleic acid, two complementary regions one having a fluorophore and one a quencher such that the molecular beacon is useful for quantifying the presence of the NRP1, NID2, ENDO180, CDK10, FPGT, CARK, PCLO or REPS2 nucleic acid in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

Anti-NRP1. NID2. ENDO180. CDK10, FPGT. CARK. PCLO or REPS2 Antibodies

The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments which can be generated by treating the antibody with an enzyme such as pepsin. An antibody sometimes is a polyclonal, monoclonal, recombinant (e.g., a chimeric or humanized), fully human, non-human (e.g., murine), or a single chain antibody. An antibody may have effector function and can fix complement, and is sometimes coupled to a toxin or imaging agent.

A full-length NRP1, NID2, ENDO180, CDK10, FPGT, CARK, PCLO or REPS2 polypeptide or antigenic peptide fragment can be used as an immunogen or can be used to identify anti-NRP1, NID2 ENDO180, CDK10, FPGT, CARK, PCLO or REPS2 antibodies made with other immunogens, e.g., cells, membrane preparations, and the like. An antigenic peptide of NRP1, NID2, ENDO180, CDK10, FPGT, CARK, PCLO or REPS2 often includes at least 8 amino acid residues of the amino acid sequences set forth in SEQ ID NO: 18-27 and encompasses an epitope of NRP1, NID2, ENDO180, CDK10, FPGT, CARK, PCLO or REPS2. Antigenic peptides sometimes include 10 or more amino acids, 15 or more amino acids, 20 or more amino acids, or 30 or more amino acids. Hydrophilic and hydrophobic fragments of NRP1, NID2, ENDO180, CDK10, FPGT, CARK, PCLO or REPS2 polypeptides sometimes are used as immunogens.

Epitopes encompassed by the antigenic peptide are regions of NRP1, NID2, ENDO180, CDK10, FPGT, CARK, PCLO or REPS2 located on the surface of the polypeptide (e.g., hydrophilic regions) as well as regions with high antigenicity. For example, an Emini surface probability analysis of the human NRP1, NID2 ENDO180, CDK10, FPGT, CARK, PCLO or REPS2 polypeptide sequence can be used to indicate the regions that have a particularly high probability of being localized to the surface of the NRP1, NID2, ENDO180, CDK10, FPGT, CARK, PCLO or REPS2 polypeptide and are thus likely to constitute surface residues useful for targeting antibody production. The antibody may bind an epitope on any domain or region on NRP1, NID2, ENDO180, CDK10, FPGT, CARK, PCLO or REPS2 polypeptides described herein.

Also, chimeric, humanized, and completely human antibodies are useful for applications which include repeated administration to subjects. Chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al International Application No. PCT/US86/02269; Akira, et al European Patent Application 184,187; Taniguchi, M., European Patent Application 171,496; Morrison et al European Patent Application 173,494; Neuberger et al PCT International Publication No. WO 86/01533; Cabilly et al U.S. Pat. No. 4,816,567; Cabilly et al European Patent Application 125,023; Better et al., Science 240: 1041-1043 (1988); Liu et al., Proc. Natl. Acad. Sci. USA 84: 3439-3443 (1987); Liu et al., J. Immunol. 139: 3521-3526 (1987); Sun et al., Proc. Natl. Acad. Sci. USA 84: 214-218 (1987); Nishimura et al., Canc. Res. 47: 999-1005 (1987); Wood et al., Nature 314: 446-449 (1985); and Shaw et al., J. Natl. Cancer Inst. 80: 1553-1559 (1988); Morrison, S. L., Science 229: 1202-1207 (1985); Oi et al., BioTechniques 4: 214 (1986); Winter U.S. Pat. No. 5,225,539; Jones et al., Nature 321: 552-525 (1986); Verhoeyan et al., Science 239: 1534; and Beidler et al., J. Immunol. 141: 4053-4060 (1988).

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar, Int. Rev. Immunol. 13: 65-93 (1995); and U.S. Pat. Nos. 5,625,126; 5,633,425; 5,569,825; 5,661,016; and 5,545,806. In addition, companies such as Abgenix, Inc. (Fremont, Calif.) and Medarex, Inc. (Princeton, N.J.), can be engaged to provide human antibodies directed against a selected antigen using technology similar to that described above. Completely human antibodies that recognize a selected epitope also can be generated using a technique referred to as “guided selection.” In this approach a selected non-human monoclonal antibody (e.g., a murine antibody) is used to guide the selection of a completely human antibody recognizing the same epitope. This technology is described for example by Jespers et al., Bio/Technology 12: 899-903 (1994).

An anti-NRP1, NID2, ENDO180, CDK10, FPGT, CARK, PCLO or REPS2 antibody can be a single chain antibody. A single chain antibody (scFV) can be engineered (see, e.g., Colcher et al., Ann. NY Acad. Sci. 880: 263-80 (1999); and Reiter, Clin. Cancer Res. 2: 245-52 (1996)). Single chain antibodies can be dimerized or multimerized to generate multivalent antibodies having specificities for different epitopes of the same target NRP1, NID2, ENDO180, CDK10, FPGT, CARK, PCLO or REPS2 polypeptide.

Antibodies also may be selected or modified so that they exhibit reduced or no ability to bind an Fc receptor. For example, an antibody may be an isotype or subtype, fragment or other mutant, which does not support binding to an Fc receptor (e.g., it has a mutagenized or deleted Fc receptor binding region).

Also, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, melphalan, carmustine (BCNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Antibody conjugates can be used for modifying a given biological response. For example, the drug moiety may be a protein or polypeptide possessing a desired biological activity. Such proteins may include, for example, a toxin such as abrin, ricin A, pseudomonas exotoxin, or diphtheria toxin; a polypeptide such as tumor necrosis factor, gamma-interferon, alpha-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Also, an antibody can be conjugated to a second antibody to form an antibody heteroconjugate as described by Segal in U.S. Pat. No. 4,676,980, for example.

An anti-NRP1, NID2, ENDO180, CDK10, FPGT, CARK, PCLO or REPS2 antibody (e.g., monoclonal antibody) can be used to isolate NRP1, NID2, ENDO180, CDK10, FPGT, CARK, PCLO or REPS2 polypeptides by standard techniques, such as affinity chromatography or immunoprecipitation. Moreover, an anti-NRP1, NID2, ENDO180, CDK10, FPGT, CARK, PCLO or REPS2 antibody can be used to detect a NRP1, NID2, ENDO180, CDK10, FPGT, CARK, PCLO or REPS2 polypeptide (e.g., in a cellular lysate or cell supernatant) in order to evaluate the abundance and pattern of expression of the polypeptide. Anti-NRP1, NID2, ENDO180, CDK10, FPGT, CARK, PCLO or REPS2 antibodies can be used diagnostically to monitor polypeptide levels in tissue as part of a clinical testing procedure, e.g., to determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, 3-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H. Also, an anti-NRP1, NID2, ENDO180, CDK10, FPGT, CARK, PCLO or REPS2 antibody can be utilized as a test molecule for determining whether it can treat melanoma, and as a therapeutic for administration to a subject for treating melanoma.

An antibody can be made by immunizing with a purified NRP1, NID2, ENDO180, CDK10, FPGT, CARK, PCLO or REPS2 antigen, or a fragment thereof, e.g., a fragment described herein, a membrane associated antigen, tissues, e.g., crude tissue preparations, whole cells, preferably living cells, lysed cells, or cell fractions.

Included herein are antibodies which bind only a native NRP1, NID2, ENDO180, CDK10, FPGT, CARK, PCLO or REPS2 polypeptide, only denatured or otherwise non-native NRP1, NID2, ENDO180, CDK10, FPGT, CARK, PCLO or REPS2 polypeptide, or which bind both, as well as those having linear or conformational epitopes. Conformational epitopes sometimes can be identified by selecting antibodies that bind to native but not denatured NRP1, NID2, ENDO180, CDK10, FPGT, CARK, PCLO or REPS2 polypeptide. Also featured are antibodies that specifically bind to a NRP1, NID2, ENDO180, CDK10, FPGT, CARK PCLO or REPS2 protein or polypeptide variant associated with melanoma. For example, the antibody sometimes specifically binds to an epitope that comprises a serine at amino acid position 656 in a NID2 protein, polypeptide or peptide.

Screening Assays

Featured herein are methods for identifying a candidate therapeutic for treating melanoma and detecting occurrence of melanoma. The methods comprise contacting a test molecule with a NRP1, NID2, ENDO180, CDK10, FPGT, CARK, PCLO or REPS2 nucleic acid, substantially identical nucleic acid, polypeptide, substantially identical polypeptide, or fragment of the foregoing in a system. The nucleic acid often is a NRP1, NID2, ENDO180, CDK10, FPGT, CARK, PCLO or REPS2 nucleotide sequence represented by SEQ ID NO: 1-17, respectively; sometimes is a nucleotide sequence substantially identical to the nucleotide sequence of SEQ ID NO: 1-17 or sometimes a fragment thereof; and the NRP1, NID2, ENDO180, CDK10, FPGT, CARK, PCLO or REPS2 polypeptide or fragment thereof is a polypeptide encoded by any of these nucleic acids. The method also comprises determining the presence or absence of an interaction between the test molecule and the NRP1, NID2, ENDO180, CDK10, FPGT, CARK, PCLO or REPS2 nucleic acid or polypeptide, where the presence of an interaction between the test molecule and the NRP1, NID2, ENDO180, CDK10, FPGT, CARK, PCLO or REPS2 nucleic acid or polypeptide identifies the test molecule as a candidate melanoma therapeutic.

As used herein, the term “test molecule” and “candidate therapeutic” refers to modulators of regulation of transcription and translation of NRP1, NID2, ENDO180, CDK10, FPGT, CARK, PCLO or REPS2 nucleic acids and modulations of expression and activity of NRP1, NID2, ENDO180, CDK10, FPGT, CARK, PCLO or REPS2 polypeptides. The term “modulator” as used herein refers to a molecule which agonizes or antagonizes NRP1, NID2, ENDO180, CDK10, FPGT, CARK, PCLO or REPS2 DNA replication and/or DNA processing (e.g., methylation), NRP1, NID2, ENDO180, CDK10, FPGT, CARK, PCLO or REPS2 RNA transcription and/or RNA processing (e.g., removal of intronic sequences and/or translocation from the nucleus), NRP1, NID2, ENDO180, CDK10, FPGT, CARK, PCLO or REPS2 polypeptide production (e.g., translation of the polypeptide from mRNA, and/or post-translational modification such as glycosylation, phosphorylation, and proteolysis of pro-polypeptides), and/or NRP1, NID2, ENDO180, CDK10, FPGT, CARK, PCLO or REPS2 function (e.g., conformational changes, binding of nucleotides or nucleotide analogs, interaction with binding partners, effect on phosphorylation, and/or effect on occurrence of melanoma). Test molecules and candidate therapeutics include, but are not limited to, compounds, RNAi or siRNA molecules, antisense nucleic acids, ribozymes, NRP1, NID2, ENDO180, CDK10, FPGT, CARK, PCLO or REPS2 polypeptides or fragments thereof, and immunotherapeutics (e.g., antibodies and HLA-presented polypeptide fragments).

As used herein, the term “system” refers to a cell free in vitro environment and a cell-based environment such as a collection of cells, a tissue, an organ, or an organism. A system is “contacted” with a test molecule in a variety of manners, including adding molecules in solution and allowing them to interact with one another by diffusion, cell injection, and any administration routes in an animal. As used herein, the term “interaction” refers to an effect of a test molecule on a NRP1, NID2, ENDO180, CDK10, FPGT, CARK, PCLO or REPS2 nucleic acid, polypeptide, or variant thereof (collectively referred to as a “NRP1, NID2, ENDO180, CDK10, FPGT, CARK, PCLO or REPS2 molecule”), where the effect is sometimes binding between the test molecule and the nucleic acid or polypeptide, and is often an observable change in cells, tissue, or organism.

There are many standard methods for detecting the presence or absence of interaction between a test molecule and a NRP1, NID2, ENDO180, CDK10, FPGT, CARK, PCLO or REPS2 nucleic acid or polypeptide. For example, titrametric, acidimetric, radiometric, NMR, monolayer, polarographic, spectrophotometric, fluorescent, and ESR assays probative of NRP1, NID2, ENDO180, CDK10, FPGT, CARK, PCLO or REPS2 function may be utilized.

NRP1, NID2, ENDO180, CDK10, FPGT, CARK, PCLO or REPS2 activity and/or NRP1, NID2, ENDO180, CDK10, FPGT, CARK, PCLO or REPS2 interactions can be detected and quantified using assays known in the art and described in Examples hereafter.

An interaction can be determined by labeling the test molecule and/or the NRP1, NID2, ENDO180, CDK10, FPGT, CARK, PCLO or REPS2 molecule, where the label is covalently or non-covalently attached to the test molecule or NRP1, NID2, ENDO180, CDK10, FPGT, CARK, PCLO or REPS2 molecule. The label is sometimes a radioactive molecule such as ¹²⁵I, ¹³¹I, ³⁵S or ³H, which can be detected by direct counting of radioemission or by scintillation counting. Also, enzymatic labels such as horseradish peroxidase, alkaline phosphatase, or luciferase may be utilized where the enzymatic label can be detected by determining conversion of an appropriate substrate to product. Also, presence or absence of an interaction can be determined without labeling. For example, a microphysiometer (e.g., Cytosensor) is an analytical instrument that measures the rate at which a cell acidifies its environment using a light-addressable potentiometric sensor (LAPS). Changes in this acidification rate can be used as an indication of an interaction between a test molecule and NRP1, NID2, ENDO180, CDK10, FPGT, CARK, PCLO or REPS2 (McConnell, H. M. et al., Science 257: 1906-1912 (1992)).

In cell-based systems, cells typically include a NRP1, NID2, ENDO180, CDK10, FPGT, CARK, PCLO or REPS2 nucleic acid or polypeptide or variants thereof and are often of mammalian origin, although the cell can be of any origin. Whole cells, cell homogenates, and cell fractions (e.g., cell membrane fractions) can be subjected to analysis. Where interactions between a test molecule with a NRP1, NID2, ENDO180, CDK10, FPGT, CARK, PCLO or REPS2 polypeptide or variant thereof are monitored, soluble and/or membrane bound forms of the polypeptide or variant may be utilized. Where membrane-bound forms of the polypeptide are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3cholamidopropyl)dimethylamminio]-2-hydroxy-1-propane sulfonate (CHAPSO), or N-dodecyl-N,N-dimethyl-3-ammonio-1-propane sulfonate.

An interaction between two molecules can also be detected by monitoring fluorescence energy transfer (FET) (see, for example, Lakowicz et al., U.S. Pat. No. 5,631,169; Stavrianopoulos et al. U.S. Pat. No. 4,868,103). A fluorophore label on a first, “donor” molecule is selected such that its emitted fluorescent energy will be absorbed by a fluorescent label on a second, “acceptor” molecule, which in turn is able to fluoresce due to the absorbed energy. Alternately, the “donor” polypeptide molecule may simply utilize the natural fluorescent energy of tryptophan residues. Labels are chosen that emit different wavelengths of light, such that the “acceptor” molecule label may be differentiated from that of the “donor”. Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. An FET binding event can be conveniently measured through standard fluorometric detection means well known in the art (e.g., using a fluorimeter).

In another embodiment, determining the presence or absence of an interaction between a test molecule and a NRP1, NID2, ENDO180, CDK10, FPGT, CARK, PCLO or REPS2 molecule can be effected by using real-time Biomolecular Interaction Analysis (BIA) (see, e.g., Sjolander & Urbaniczk, Anal. Chem. 63: 2338-2345 (1991) and Szabo et al., Curr. Opin. Struct. Biol. 5: 699-705 (1995)). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In another embodiment, the NRP1, NID2, ENDO180, CDK10, FPGT, CARK, PCLO or REPS2 molecule or test molecules are anchored to a solid phase. The NRP1, NID2, ENDO180, CDK10, FPGT, CARK, PCLO or REPS2 molecule/test molecule complexes anchored to the solid phase can be detected at the end of the reaction. The target NRP1, NID2, ENDO180, CDK10, FPGT, CARK, PCLO or REPS2 molecule is often anchored to a solid surface, and the test molecule, which is not anchored, can be labeled, either directly or indirectly, with detectable labels discussed herein.

It may be desirable to immobilize a NRP1, NID2, ENDO180, CDK10, FPGT, CARK, PCLO or REPS2 molecule, an anti-NRP1, NID2 ENDO180, CDK10, FPGT, CARK, PCLO or REPS2 antibody, or test molecules to facilitate separation of complexed from uncomplexed forms of NRP1, NID2, ENDO180, CDK10, FPGT, CARK, PCLO or REPS2 molecules and test molecules, as well as to accommodate automation of the assay. Binding of a test molecule to a NRP1, NID2, ENDO180, CDK10, FPGT, CARK, PCLO or REPS2 molecule can be accomplished in any vessel suitable for containing the reactants. Examples of such vessels include microtitre plates, test tubes, and micro-centrifuge tubes. In one embodiment, a fusion polypeptide can be provided which adds a domain that allows a NRP1, NID2, ENDO180, CDK10, FPGT, CARK, PCLO or REPS2 molecule to be bound to a matrix. For example, glutathione-5-transferase/NRP1, NID2, ENDO180, CDK10, FPGT, CARK, PCLO or REPS2 fusion polypeptides or glutathione-5-transferase/target fusion polypeptides can be adsorbed onto glutathione sepharose beads (Sigma Chemical, St. Louis, Mo.) or glutathione derivatized microtitre plates, which are then combined with the test compound or the test compound and either the non-adsorbed target polypeptide or NRP1, NID2, ENDO180, CDK10, FPGT, CARK, PCLO or REPS2 polypeptide, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of NRP1, NID2, ENDO180, CDK10, FPGT, CARK, PCLO or REPS2 binding or activity determined using standard techniques.

Other techniques for immobilizing a NRP1, NID2, ENDO180, CDK10, FPGT, CARK, PCLO or REPS2 molecule on matrices include using biotin and streptavidin. For example, biotinylated NRP1, NID2, ENDO180, CDK10, FPGT, CARK, PCLO or REPS2 polypeptide or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with NRP1, NID2, ENDO180, CDK10, FPGT, CARK, PCLO or REPS2 polypeptide or test molecules but which do not interfere with binding of the NRP1, NID2 ENDO180, CDK10, FPGT, CARK, PCLO or REPS2 polypeptide to its test molecule. Such antibodies can be derivatized to the wells of the plate, and unbound target or NRP1, NID2, ENDO180, CDK10, FPGT, CARK, PCLO or REPS2 polypeptide trapped in the wells by antibody conjugation. Methods for detecting such complexes, in addition to those described above for the GST-immobilized complexes, include immunodetection of complexes using antibodies reactive with the NRP1, NID2, ENDO180, CDK10, FPGT, CARK, PCLO or REPS2 polypeptide or target molecule, as well as enzyme-linked assays which rely on detecting an enzymatic activity associated with the NRP1, NID2, ENDO180, CDK10, FPGT, CARK, PCLO or REPS2 polypeptide or test molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., Trends Biochem Sci August; 18(8): 284-7 (1993)); chromatography (gel filtration chromatography, ion-exchange chromatography); electrophoresis (see, e.g., Ausubel et al., eds. Current Protocols in Molecular Biology, J Wiley: New York (1999)); and immunoprecipitation (see, for example, Ausubel, F. et al., eds. Current Protocols in Molecular Biology, J. Wiley: New York (1999)). Such resins and chromatographic techniques are known to one skilled in the art (see, e.g., Heegaard, J. Mol. Recognit. Winter; 11(1-6): 141-8 (1998); Hage & Tweed, J. Chromatogr. B Biomed. Sci. Appl. October 10; 699 (1-2): 499-525 (1997)). Further, fluorescence energy transfer may also be conveniently utilized, as described herein, to detect binding without further purification of the complex from solution.

In another embodiment, modulators of NRP1, NID2, ENDO180, CDK10, FPGT, CARK, PCLO or REPS2 expression are identified. For example, a cell or cell free mixture is contacted with a candidate compound and the expression of NRP1, NID2, ENDO180, CDK10, FPGT, CARK, PCLO or REPS2 mRNA or polypeptide evaluated relative to the level of expression of NRP1, NID2, ENDO180, CDK10, FPGT, CARK, PCLO or REPS2 mRNA or polypeptide in the absence of the candidate compound. When expression of NRP1, NID2, ENDO180, CDK10, FPGT, CARK, PCLO or REPS2 mRNA or polypeptide is greater in the presence of the candidate compound than in its absence, the candidate compound is identified as a stimulator of NRP1, NID2, ENDO180, CDK10, FPGT, CARK, PCLO or REPS2 mRNA or polypeptide expression. Alternatively, when expression of NRP1, NID2, ENDO180, CDK10, FPGT, CARK, PCLO or REPS2 mRNA or polypeptide is less (statistically significantly less) in the presence of the candidate compound than in its absence, the candidate compound is identified as an inhibitor of NRP1, NID2, ENDO180, CDK10, FPGT, CARK, PCLO or REPS2 mRNA or polypeptide expression. The level of NRP1, NID2, ENDO180, CDK10, FPGT, CARK, PCLO or REPS2 mRNA or polypeptide expression can be determined by methods described herein for detecting NRP1, NID2, ENDO180, CDK10, FPGT, CARK, PCLO or REPS2 mRNA or polypeptide.

In an embodiment, binding partners that interact with a NRP1, NID2, ENDO180, CDK10, FPGT, CARK, PCLO or REPS2 molecule are detected. The NRP1, NID2, ENDO180, CDK10, FPGT, CARK, PCLO or REPS2 molecules can interact with one or more cellular or extracellular macromolecules, such as polypeptides, in vivo, and these molecules that interact with NRP1, NID2, ENDO180, CDK10, FPGT, CARK, PCLO or REPS2 molecules are referred to herein as “binding partners.” Molecules that disrupt such interactions can be useful in regulating the activity of the target gene product. Such molecules can include, but are not limited to molecules such as antibodies, peptides, and small molecules (e.g., siRNA). The preferred target genes/products for use in this embodiment are the NRP1, NID2, ENDO180, CDK10, FPGT, CARK, PCLO or REPS2 genes herein identified. In an alternative embodiment, provided are methods for determining the ability of the test compound to modulate the activity of a NRP1, NID2 ENDO180, CDK10, FPGT, CARK, PCLO or REPS2 polypeptide through modulation of the activity of a downstream effector of a NRP1, NID2, ENDO180, CDK10, FPGT, CARK, PCLO or REPS2 target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), e.g., a substrate, a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtitre plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

Also, binding partners of NRP1, NID2, ENDO180, CDK10, FPGT, CARK, PCLO or REPS2 molecules can be identified in a two-hybrid assay or three-hybrid assay (see, e.g., U.S. Pat. No. 5,283,317; Zervos et al., Cell 72: 223-232 (1993); Madura et al., J. Biol. Chem. 268: 12046-12054 (1993); Bartel et al., Biotechniques 14: 920-924 (1993); Iwabuchi et al., Oncogene 8: 1693-1696 (1993); and Brent WO94/10300), to identify other polypeptides, which bind to or interact with NRP1, NID2, ENDO180, CDK10, FPGT, CARK, PCLO or REPS2 (“NRP1, NID2, ENDO180, CDK10, FPGT, CARK, PCLO or REPS2-binding polypeptides” or “NRP1, NID2, ENDO180, CDK10, FPGT, CARK, PCLO or REPS2-bp”) and are involved in NRP1, NID2, ENDO180, CDK10, FPGT, CARK, PCLO or REPS2 activity. Such NRP1, NID2, ENDO180, CDK10, FPGT, CARK, PCLO or REPS2-bps can be activators or inhibitors of signals by the NRP1, NID2, ENDO180, CDK10, FPGT, CARK, PCLO or REPS2 polypeptides or NRP1, NID2, ENDO180, CDK10, FPGT, CARK, PCLO or REPS2 targets as, for example, downstream elements of a NRP1, NID2, ENDO180, CDK10, FPGT, CARK, PCLO or REPS2-mediated signaling pathway.

A two-hybrid system is based on the modular nature of most transcription factors, which consist of separable DNA-binding and activation domains. Briefly, the assay utilizes two different DNA constructs. In one construct, the gene that codes for a NRP1, NID2, ENDO180, CDK10, FPGT, CARK, PCLO or REPS2 polypeptide is fused to a gene encoding the DNA binding domain of a known transcription factor (e.g., GAL-4). In the other construct, a DNA sequence, from a library of DNA sequences, that encodes an unidentified polypeptide (“prey” or “sample”) is fused to a gene that codes for the activation domain of the known transcription factor. (Alternatively the: NRP1, NID2, ENDO180, CDK10, FPGT, CARK, PCLO or REPS2 polypeptide can be the fused to the activator domain.) If the “bait” and the “prey” polypeptides are able to interact, in vivo, forming a NRP1, NID2, ENDO180, CDK10, FPGT, CARK, PCLO or REPS2-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the polypeptide which interacts with the NRP1, NID2, ENDO180, CDK10, FPGT, CARK, PCLO or REPS2 polypeptide.

Candidate therapeutics for treating melanoma are identified from a group of test molecules that interact with a NRP1, NID2, ENDO180, CDK10, FPGT, CARK, PCLO or REPS2 nucleic acid or polypeptide. Test molecules often are ranked according to the degree with which they interact or modulate (e.g., agonize or antagonize) DNA replication and/or processing, RNA transcription and/or processing, polypeptide production and/or processing, and/or function of NRP1, NID2, ENDO180, CDK10, FPGT, CARK, PCLO or REPS2 molecules, for example, and then top ranking modulators are selected. Also, pharmacogenomic information described herein can determine the rank of a modulator. Candidate therapeutics typically are formulated for administration to a subject.

Therapeutic Treatments

Formulations or pharmaceutical compositions typically include in combination with a pharmaceutically acceptable carrier a compound, an antisense nucleic acid, a ribozyme, an antibody, a binding partner that interacts with a NRP1, NID2, ENDO180, CDK10, FPGT, CARK, PCLO or REPS2 polypeptide, a NRP1, NID2, ENDO180, CDK10, FPGT, CARK, PCLO or REPS2 nucleic acid, or a fragment thereof. The formulated molecule may be one that is identified by a screening method described above. Also, formulations may comprise a NRP1, NID2, ENDO180, CDK10, FPGT, CARK, PCLO or REPS2 polypeptide or fragment thereof and a pharmaceutically acceptable carrier. As used herein, the term “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the methods of preparation often utilized are vacuum drying and freeze-drying which yields a powder of the active ingredient plus any additional desired ingredient from a previously sterile-filtered solution thereof.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams (e.g., sunscreen) as generally known in the art. Molecules can also be prepared in the form of suppositories (e.g., with conventional suppository bases such as cocoa butter and other glycerides) or retention enemas for rectal delivery.

In one embodiment, active molecules are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Molecules which exhibit high therapeutic indices often are utilized. While molecules that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such molecules lies preferably within a range of circulating concentrations that include the ED₅₀ with little or no toxicity. The dosage may vary within this range depending upon the dosage form employed and the route of administration utilized. For any molecules used in the method, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, sometimes about 0.01 to 25 mg/kg body weight, often about 0.1 to 20 mg/kg body weight, and more often about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, sometimes between 2 to 8 weeks, often between about 3 to 7 weeks, and more often for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

With regard to polypeptide formulations, featured herein is a method for treating melanoma in a subject, which comprises contacting one or more cells in the subject with a first NRP1, NID2, ENDO180, CDK10, FPGT, CARK, PCLO or REPS2 polypeptide, where genomic DNA in the subject comprises a second NRP1, NID2, ENDO180, CDK10, FPGT, CARK, PCLO or REPS2 nucleic acid having one or more polymorphic variations associated with melanoma. The first NRP1, NID2, ENDO180, CDK10, FPGT, CARK, PCLO or REPS2 polypeptide comprises fewer polymorphic variations associated with melanoma than the second NRP1, NID2, ENDO180, CDK10, FPGT, CARK, PCLO or REPS2 polypeptide. The first and second NRP1, NID2, ENDO180, CDK10, FPGT, CARK, PCLO or REPS2 polypeptides are encoded by a nucleic acid which comprises a nucleotide sequence selected from the group consisting of the nucleotide sequence of SEQ ID NO: 1-17; a nucleotide sequence which encodes a polypeptide consisting of an amino acid sequence set forth in SEQ ID NO: 18-27; and a nucleotide sequence which encodes a polypeptide that is 90% or more identical to an amino acid sequence set forth in SEQ ID NO: 18-27. The second NRP1, NID2, ENDO180, CDK10, FPGT, CARK, PCLO or REPS2 polypeptide also may be encoded by a fragment of the foregoing nucleic acids comprising the one or more polymorphic variations. The subject is often a human.

For antibodies, a dosage of 0.1 mg/kg of body weight (generally 10 mg/kg to 20 mg/kg) is often utilized. If the antibody is to act in the brain, a dosage of 50 mg/kg to 100 mg/kg is often appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al., J. Acquired Immune Deficiency Syndromes and Human Retrovirology 14: 193 (1997).

Antibody conjugates can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a polypeptide such as tumor necrosis factor, .alpha.-interferon, .beta.-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Alternatively, an antibody can be conjugated to a second antibody to form an antibody heteroconjugate as described by Segal in U.S. Pat. No. 4,676,980.

For compounds, exemplary doses include milligram or microgram amounts of the compound per kilogram of subject or sample weight, for example, about 1 microgram per kilogram to about 500 milligrams per kilogram, about 100 micrograms per kilogram to about 5 milligrams per kilogram, or about 1 microgram per kilogram to about 50 micrograms per kilogram. It is understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

NRP1, NID2, ENDO180, CDK10, FPGT, CARK, PCLO or REPS2 nucleic acid molecules can be inserted into vectors and used in gene therapy methods for treating melanoma. Featured herein is a method for treating melanoma in a subject, which comprises contacting one or more cells in the subject with a first NRP1, NID2, ENDO180, CDK10, FPGT, CARK, PCLO or REPS2 nucleic acid. Genomic DNA in the subject comprises a second NRP1, NID2, ENDO180, CDK10, FPGT, CARK, PCLO or REPS2 nucleic acid comprising one or more polymorphic variations associated with melanoma, and the first NRP1, NID2, ENDO180, CDK10, FPGT, CARK, PCLO or REPS2 nucleic acid comprises fewer polymorphic variations associated with melanoma. The first and second NRP1, NID2, ENDO180, CDK10, FPGT, CARK, PCLO or REPS2 nucleic acids typically comprise a nucleotide sequence selected from the group consisting of the nucleotide sequence of SEQ ID NO: 1-17; a nucleotide sequence which encodes a polypeptide consisting of an amino acid sequence set forth in SEQ ID NO: 18-27; and a nucleotide sequence which encodes a polypeptide that is 90% or more identical to an amino acid sequence set forth in SEQ ID NO: 18-27. The second NRP1, NID2 ENDO180, CDK10, FPGT, CARK, PCLO or REPS2 nucleic acid may also be a fragment of the foregoing comprising one or more polymorphic variations. The subject is often a human.

Gene therapy vectors can be delivered to a subject by, for example, intravenous injection, local administration (see U.S. Pat. No. 5,328,470) or by stereotactic injection (see e.g., Chen et al., (1994) Proc. Natl. Acad. Sci. USA 91: 3054-3057). Pharmaceutical preparations of gene therapy vectors can include a gene therapy vector in an acceptable diluent, or can comprise a slow release matrix in which the gene delivery vehicle is imbedded. Alternatively, where the complete gene delivery vector can be produced intact from recombinant cells (e.g., retroviral vectors) the pharmaceutical preparation can include one or more cells which produce the gene delivery system. Examples of gene delivery vectors are described herein.

Pharmaceutical compositions can be included in a container, pack, or dispenser together with instructions for administration.

Pharmaceutical compositions of active ingredients can be administered by any of the paths described herein for therapeutic and prophylactic methods for treating melanoma. With regard to both prophylactic and therapeutic methods of treatment, such treatments may be specifically tailored or modified, based on knowledge obtained from pharmacogenomic analyses described herein. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides.

Administration of a prophylactic agent can occur prior to the manifestation of symptoms characteristic of the NRP1, NID2, ENDO180, CDK10, FPGT, CARK, PCLO or REPS2 aberrance, such that a disease or disorder is prevented or, alternatively, delayed in its progression. Depending on the type of NRP1, NID2, ENDO180, CDK10, FPGT, CARK, PCLO or REPS2 aberrance, for example, a NRP1, NID2, ENDO180, CDK10, FPGT, CARK, PCLO or REPS2 molecule, NRP1, NID2, ENDO180, CDK10, FPGT, CARK, PCLO or REPS2 agonist, or NRP1, NID2, ENDO180, CDK10, FPGT, CARK, PCLO or REPS2 antagonist agent can be used for treating the subject. The appropriate agent can be determined based on screening assays described herein.

As discussed, successful treatment of NRP1, NID2, ENDO180, CDK10, FPGT, CARK, PCLO or REPS2 disorders can be brought about by tech niques that serve to inhibit the expression or activity of target gene products. For example, compounds (e.g., an agent identified using an assays described above) that exhibit negative modulatory activity can be used to prevent and/or treat melanoma. Such molecules can include, but are not limited to peptides, phosphopeptides, small organic or inorganic molecules, or antibodies (including, for example, polyclonal, monoclonal, humanized, anti-idiotypic, chimeric or single chain antibodies, and FAb, F(ab′)₂ and FAb expression library fragments, scFV molecules, and epitope-binding fragments thereof).

Further, antisense and ribozyme molecules that inhibit expression of the target gene can also be used to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular polypeptide, it can be preferable to co-administer normal target gene polypeptide into the cell or tissue in order to maintain the requisite level of cellular or tissue target gene activity.

Another method by which nucleic acid molecules may be utilized in treating or preventing a disease characterized by NRP1, NID2, ENDO180, CDK10, FPGT, CARK, PCLO or REPS2 expression is through the use of aptamer molecules specific for NRP1, NID2, ENDO180, CDK10, FPGT, CARK, PCLO or REPS2 polypeptide. Aptamers are nucleic acid molecules having a tertiary structure which permits them to specifically bind to polypeptide ligands (see, e.g., Osborne, et al., Curr. Opin. Chem. Biol. 1 (1): 5-9 (1997); and Patel, D. J., Curr. Opin. Chem. Biol. June; 1 (1): 3246 (1997)). Since nucleic acid molecules may in many cases be more conveniently introduced into target cells than therapeutic polypeptide molecules may be, aptamers offer a method by which NRP1, NID2, ENDO180, CDK10, FPGT, CARK, PCLO or REPS2 polypeptide activity may be specifically decreased without the introduction of drugs or other molecules which may have pluripotent effects.

Antibodies can be generated that are both specific for target gene product and that reduce target gene product activity. Such antibodies may, therefore, by administered in instances whereby negative modulatory techniques are appropriate for the treatment of NRP1, NID2, ENDO180, CDK10, FPGT, CARK, PCLO or REPS2 disorders. For a description of antibodies, see the Antibody section above.

In circumstances where injection of an animal or a human subject with a NRP1, NID2, ENDO180, CDK10, FPGT, CARK, PCLO or REPS2 polypeptide or epitope for stimulating antibody production is harmful to the subject, it is possible to generate an immune response against NRP1, NID2, ENDO180, CDK10, FPGT, CARK, PCLO or REPS2 through the use of anti-idiotypic antibodies (see, for example, Herlyn, D., Ann. Med.; 31(1): 66-78 (1999); and Bhattacharya-Chatterjee & Foon, Cancer Treat. Res.; 94: 51-68 (1998)). If an anti-idiotypic antibody is introduced into a mammal or human subject, it should stimulate the production of anti-anti-idiotypic antibodies, which should be specific to the NRP1, NID2, ENDO180, CDK10, FPGT, CARK, PCLO or REPS2 polypeptide. Vaccines directed to a disease characterized by NRP1, NID2, ENDO180, CDK10, FPGT, CARK, PCLO or REPS2 expression may also be generated in this fashion.

In instances where the target antigen is intracellular and whole antibodies are used, internalizing antibodies often are utilized. Lipofectin or liposomes can be used to deliver the antibody or a fragment of the Fab region that binds to the target antigen into cells. Where fragments of the antibody are used, the smallest inhibitory fragment that binds to the target antigen often are utilized. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al., Proc. Natl. Acad. Sci. USA 90: 7889-7893 (1993)).

NRP1, NID2, ENDO180, CDK10, FPGT, CARK, PCLO or REPS2 molecules and compounds that inhibit target gene expression, synthesis and/or activity can be administered to a patient at therapeutically effective doses to prevent, treat or ameliorate NRP1, NID2, ENDO180, CDK10, FPGT, CARK, PCLO or REPS2 disorders. A therapeutically effective dose refers to that amount of the compound sufficient to result in amelioration of symptoms of the disorders.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices often are utilized. While compounds that exhibit toxic side effects can be used, care should be taken to design a delivery system that targets such compounds to the site of affected tissue in order to minimize potential damage to uninfected cells and, thereby, reduce side effects.

Data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of effective dose determination for an individual is the ability to directly assay levels of “free” and “bound” compound in the serum of the test subject. Such assays may utilize antibody mimics and/or “biosensors” that have been created through molecular imprinting techniques.

The compound which is able to modulate NRP1, NID2, ENDO180, CDK10, FPGT, CARK, PCLO or REPS2 activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell et al., Current Opinion in Biotechnology 7: 89-94 (1996) and in Shea, Trends in Polymer Science 2: 166-173 (1994). Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, et al., Nature 361: 645-647 (1993). Through the use of isotope-labeling, the “free” concentration of compound which modulates the expression or activity of NRP1, NID2, ENDO180, CDK10, FPGT, CARK, PCLO or REPS2 can be readily monitored and used in calculations of IC₅₀. Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. A rudimentary example of such a “biosensor” is discussed in Kriz et al., Analytical Chemistry 67: 2142-2144 (1995).

Provided herein are methods of modulating NRP1, NID2, ENDO180, CDK10, FPGT, CARK, PCLO or REPS2 expression or activity for therapeutic purposes. Accordingly, in an exemplary embodiment, the modulatory method involves contacting a cell with NRP1, NID2, ENDO180, CDK10, FPGT, CARK, PCLO or REPS2 or an agent that modulates one or more activities of NRP1, NID2, ENDO180, CDK10, FPGT, CARK, PCLO or REPS2 polypeptide activity associated with the cell. An agent that modulates NRP1, NID2, ENDO180, CDK10, FPGT, CARK, PCLO or REPS2 polypeptide activity can be an agent as described herein, such as a nucleic acid or a polypeptide, a naturally-occurring target molecule of a NRP1, NID2, ENDO180, CDK10, FPGT, CARK, PCLO or REPS2 polypeptide (e.g., a NRP1, NID2, ENDO180, CDK10, FPGT, CARK, PCLO or REPS2 substrate or receptor), a NRP1, NID2, ENDO180, CDK10, FPGT, CARK, PCLO or REPS2 antibody, a NRP1, NID2, ENDO180, CDK10, FPGT, CARK, PCLO or REPS2 agonist or antagonist, a peptidomimetic of a NRP1, NID2, ENDO180, CDK10, FPGT, CARK, PCLO or REPS2 agonist or antagonist, or other small molecule.

In one embodiment, the agent stimulates one or more NRP1, NID2, ENDO180, CDK10, FPGT, CARK, PCLO or REPS2 activities. Examples of such stimulatory agents include active NRP1, NID2 ENDO180, CDK10, FPGT, CARK, PCLO or REPS2 polypeptide and a nucleic acid molecule encoding NRP1, NID2, ENDO180, CDK10, FPGT, CARK, PCLO or REPS2. In another embodiment, the agent inhibits one or more NRP1, NID2, ENDO180, CDK10, FPGT, CARK, PCLO or REPS2 activities. Examples of such inhibitory agents include antisense NRP1, NID2, ENDO180, CDK10, FPGT, CARK, PCLO or REPS2 nucleic acid molecules, anti-NRP1, NID2, ENDO180, CDK10, FPGT, CARK, PCLO or REPS2 antibodies, and NRP1, NID2, ENDO180, CDK10, FPGT, CARK, PCLO or REPS2 inhibitors. These modulatory methods can be performed in vitro (e.g., by culturing the cell with the agent) or, alternatively, in vivo (e.g., by administering the agent to a subject). As such, provided are methods of treating an individual afflicted with a disease or disorder characterized by aberrant or unwanted expression or activity of a NRP1, NID2, ENDO180, CDK10, FPGT, CARK, PCLO or REPS2 polypeptide or nucleic acid molecule. In one embodiment, the method involves administering an agent (e.g., an agent identified by a screening assay described herein), or combination of agents that modulates (e.g., upregulates or downregulates) NRP1, NID2, ENDO180, CDK10, FPGT, CARK, PCLO or REPS2 expression or activity. In another embodiment, the method involves administering a NRP1, NID2, ENDO180, CDK10, FPGT, CARK, PCLO or REPS2 polypeptide or nucleic acid molecule as therapy to compensate for reduced, aberrant, or unwanted NRP1, NID2, ENDO180, CDK10, FPGT, CARK, PCLO or REPS2 expression or activity.

Stimulation of NRP1, NID2, ENDO180, CDK10, FPGT, CARK, PCLO or REPS2 activity is desirable in situations in which NRP1, NID2, ENDO180, CDK10, FPGT, CARK, PCLO or REPS2 is abnormally downregulated and/or in which increased NRP1, NID2, ENDO180, CDK10, FPGT, CARK, PCLO or REPS2 activity is likely to have a beneficial effect. For example, stimulation of NRP1, NID2, ENDO180, CDK10, FPGT, CARK, PCLO or REPS2 activity is desirable in situations in which a NRP1, NID2, ENDO180, CDK10, FPGT, CARK, PCLO or REPS2 is downregulated and/or in which increased NRP1, NID2, ENDO180, CDK10, FPGT, CARK, PCLO or REPS2 activity is likely to have a beneficial effect. Likewise, inhibition of NRP1, NID2, ENDO180, CDK10, FPGT, CARK, PCLO or REPS2 activity is desirable in situations in which NRP1, NID2, ENDO180, CDK10, FPGT, CARK, PCLO or REPS2 is abnormally upregulated and/or in which decreased NRP1, NID2, ENDO180, CDK10, FPGT, CARK, PCLO or REPS2 activity is likely to have a beneficial effect.

The examples set forth below are intended to illustrate but not limit the invention.

### EXAMPLES

In the following studies a group of subjects were selected according to specific parameters relating to melanoma. Nucleic acid samples obtained from individuals in the study group were subjected to genetic analysis, which identified associations between melanoma and certain polymorphic regions in the NRP1, NID2, ENDO180, CDK10, FPGT, CARK, PCLO or REPS2 genes. Methods are described for producing NRP1, NID2, ENDO180, CDK10, FPGT, CARK, PCLO or REPS2 polypeptides and polypeptide variants in vitro or in vivo. NRP1, NID2, ENDO180, CDK10, FPGT, CARK, PCLO or REPS2 nucleic acids or polypeptides and variants thereof are utilized for screening test molecules for those that interact with NRP1, NID2, ENDO180, CDK10, FPGT, CARK, PCLO or REPS2 molecules. Test molecules identified as interactors with NRP1, NID2, ENDO180, CDK10, FPGT, CARK, PCLO or REPS2 molecules and variants are further screened in vivo to determine whether they treat melanoma.

### Example 1

### Samples and Pooling Strategies

Sample Selection

Blood samples were collected from individuals diagnosed with melanoma, which were referred to as case samples. Also, blood samples were collected from individuals not diagnosed with melanoma or a history of melanoma; these samples served as gender and age-matched controls. A database was created that listed all phenotypic trait information gathered from individuals for each case and control sample. Genomic DNA was extracted from each of the blood samples for genetic analyses.

DNA Extraction from Blood Samples

Six to ten milliliters of whole blood was transferred to a 50 ml tube containing 27 ml of red cell lysis solution (RCL). The tube was inverted until the contents were mixed. Each tube was incubated for 10 minutes at room temperature and inverted once during the incubation. The tubes were then centrifuged for 20 minutes at 3000×g and the supernatant was carefully poured off. 100-200 μl of residual liquid was left in the tube and was pipetted repeatedly to resuspend the pellet in the residual supernatant. White cell lysis solution (WCL) was added to the tube and pipetted repeatedly until completely mixed. While no incubation was normally required, the solution was incubated at 37° C. or room temperature if cell clumps were visible after mixing until the solution was homogeneous. Two ml of protein precipitation was added to the cell lysate. The mixtures were vortexed vigorously at high speed for 20 sec to mix the protein precipitation solution uniformly with the cell lysate, and then centrifuged for 10 minutes at 3000×g. The supernatant containing the DNA was then poured into a clean 15 ml tube, which contained 7 ml of 100% isopropanol. The samples were mixed by inverting the tubes gently until white threads of DNA were visible. Samples were centrifuged for 3 minutes at 2000×g and the DNA was visible as a small white pellet. The supernatant was decanted and 5 ml of 70% ethanol was added to each tube. Each tube was inverted several times to wash the DNA pellet, and then centrifuged for 1 minute at 2000×g. The ethanol was decanted and each tube was drained on clean absorbent paper. The DNA was dried in the tube by inversion for 10 minutes, and then 1000 μl of 1×TE was added. The size of each sample was estimated, and less TE buffer was added during the following DNA hydration step if the sample was smaller. The DNA was allowed to rehydrate overnight at room temperature, and DNA samples were stored at 2-8° C.

DNA was quantified by placing samples on a hematology mixer for at least 1 hour. DNA was serially diluted (typically 1:80, 1:160, 1:320, and 1:640 dilutions) so that it would be within the measurable range of standards. 125 μl of diluted DNA was transferred to a clear U-bottom microtitre plate, and 125 μl of 1×TE buffer was transferred into each well using a multichannel pipette. The DNA and 1×TE were mixed by repeated pipetting at least 15 times, and then the plates were sealed. 50 μl of diluted DNA was added to wells A5-H12 of a black flat bottom microtitre plate. Standards were inverted six times to mix them, and then 50 μl of 1×TE buffer was pipetted into well A1, 1000 ng/ml of standard was pipetted into well A2, 500 ng/ml of standard was pipetted into well A3, and 250 ng/ml of standard was pipetted into well A4. PicoGreen (Molecular Probes, Eugene, Oreg.) was thawed and freshly diluted 1:200 according to the number of plates that were being measured. PicoGreen was vortexed and then 501 μl was pipetted into all wells of the black plate with the diluted DNA. DNA and PicoGreen were mixed by pipetting repeatedly at least 10 times with the multichannel pipette. The plate was placed into a Fluoroskan Ascent Machine (microplate fluorometer produced by Labsystems) and the samples were allowed to incubate for 3 minutes before the machine was run using filter pairs 485 nm excitation and 538 nm emission wavelengths. Samples having measured DNA concentrations of greater than 450 ng/μl were re-measured for conformation. Samples having measured DNA concentrations of 20 ng/μl or less were re-measured for confirmation.

Pooling Strategies

Samples were placed into one of four groups, based on gender and disease status. The four groups were male case samples, male control samples, female case samples, and female control samples. A select set of samples from each group were utilized to generate pools, and one pool was created for each group. Each individual sample in a pool was represented by an equal amount of genomic DNA. For example, where 25 ng of genomic DNA was utilized in each PCR reaction and there were 200 individuals in each pool, each individual would provide 125 pg of genomic DNA. Inclusion or exclusion of samples for a pool was based upon the following criteria: the sample was derived from an individual characterized as Caucasian; the sample was derived from an individual of German paternal and maternal descent; the database included relevant phenotype information for the individual; case samples were derived from individuals diagnosed with melanoma; control samples were derived from individuals free of cancer; and sufficient genomic DNA was extracted from each blood sample for all allelotyping and genotyping reactions performed during the study. Phenotype information included sex of the individual, number of nevi (few, moderate, numerous), hair color (black, brown, blond, red), diagnosed with melanoma (tumor thickness, date of primary diagnosis, age of individual as of primary diagnosis, post-operative tumor classification, presence of nodes, occurrence of metastases, subtype, location), country or origin of mother and father, presence of certain conditions for each individual (coronary heart disease, cardiomyopathy, arteriosclerosis, abnormal blood clotting/thrombosis, emphysema, asthma, diabetes type 1, diabetes type 2, Alzheimer's disease, epilepsy, schizophrenia, manic depression/bipolar disorder, autoimmune disease, thyroid disorder, and hypertension), presence of cancer in the donor individual or blood relative (melanoma, basaliom/spinaliom/lentigo malignant/mycosis fungoides, breast cancer, colon cancer, rectum cancer, lung cancer, lung cancer, bronchus cancer, prostate cancer, stomach cancer, leukemia, lymphoma, or other cancer in donor, donor parent, donor aunt or uncle, donor offspring or donor grandparent. Samples that met these criteria were added to appropriate pools based on gender and disease status.

The selection process yielded the pools set forth in Table 1, which were used in the studies that follow:

TABLE 1

| | Male | Male | Female | Female |
|---|---|---|---|---|
| | control | case | control | case |
| Pool size (Number) | 217 | 236 | 233 | 266 |
| Pool Criteria | control | case | control | case |
| (ex: case/control) | | | | |
| Mean Age (ex: years) | 48 | 51 | 47 | 49 |

### Example 2

### Association of Polymorphic Variants with Melanoma

A whole-genome screen was performed to identify particular SNPs associated with occurrence of melanoma. As described in Example 1, two sets of samples were utilized: female individuals having melanoma (female cases) and samples from female individuals not having melanoma or any history of melanoma (female controls), and male individuals having melanoma (male cases) and samples from male individuals not having melanoma or any history of melanoma (male controls). The initial screen of each pool was performed in an allelotyping study, in which certain samples in each group were pooled. By pooling DNA from each group, an allele frequency for each SNP in each group was calculated. These allele frequencies were then compared to one another. Particular SNPs were considered as being associated with melanoma when allele frequency differences calculated between case and control pools were statistically significant. SNP disease association results obtained from the allelotyping study were then validated by genotyping each associated SNP across all samples from each pool. The results of the genotyping were then analyzed, allele frequencies for each group were calculated from the individual genotyping results, and a p-value was calculated to determine whether the case and control groups had statistically significantly differences in allele frequencies for a particular SNP. When the genotyping results agreed with the original allelotyping results, the SNP disease association was considered validated at the genetic level.

SNP Panel Used for Genetic Analyses

A whole-genome SNP screen began with an initial screen of approximately 25,000 SNPs over each set of disease and control samples using a pooling approach. The pools studied in the screen are described in Example 1. The SNPs analyzed in this study were part of a set of 25,488 SNPs confirmed as being statistically polymorphic as each is characterized as having a minor allele frequency of greater than 10%. The SNPs in the set reside in genes or in close proximity to genes, and many reside in gene exons. Specifically, SNPs in the set are located in exons, introns, and within 5,000 base-pairs upstream of a transcription start site of a gene. In addition, SNPs were selected according to the following criteria: they are located in ESTs; they are located in Locuslink or Ensembl genes; and they are located in Genomatix promoter predictions. SNPs in the set were also selected on the basis of even spacing across the genome, as depicted in Table 2.

TABLE 2

| General Statistics | | Spacing Statistics | | |
|---|---|---|---|---|
| Total # of SNPs | 28,532 | Median | 34,424 | bp |
| # of Exonic SNPs | 8,497 (30%) | Minimum* | 1,000 | bp |
| # SNPs with | 26,625 (93%) | Maximum* | 3,000,000 | bp |
| refSNP ID | | | | |
| Gene Coverage | 23,874 | Mean | 122,412 | bp |
| Chromosome | All | Std | 354, | bp |
| Coverage | | Deviation | | |

*Excludes outliers

Genotyping Results

The genetic studies summarized above and described in more detail below identified allelic variants associated with melanoma. The allelic variants identified from the SNP panel described in Table 2 are summarized below in Table 3.

TABLE 3

| | | | | | | | Melanoma |
|---|---|---|---|---|---|---|---|
| SNP | Chromo- | Contig | Contig | Sequence | Sequence | Allelic | Associated |
| Reference | some | Identification | Position | Identification | Position | Variability | Allele |
| rs1360457 | 10 | NT_008862 | 15592254 | NM_003873 | intronic | T/C | T |
| rs753050 | 14 | NT_026437 | 32425413 | NM_007361 | intronic | A/G | A |
| rs729647 | 17 | NT_010940 | 19400937 | NM_006039 | intronic | A/G | G |
| rs8404 | 16 | 89506442 | NT_010542 | 1320117 | NM_003674 | UTR | C/T |
| rs1044639 | 7 | 82063609 | NT_007933 | 7685796 | XM_168530 | UTR | C/A |
| rs2034453 | 1 | 74082116 | NT_032977 | 36275269 | NM_015978 | intronic | A/G |
| rs2034453 | 1 | | NT_032977 | | NM_003838 | intragenic | |
| rs1904528 | X | 12970302 | NT_011757 | 12970302 | NM_004726 | UTR | C/T |

Table 3 includes information pertaining to the incident polymorphic variant associated with melanoma identified herein. Public information pertaining to the polymorphism and the genomic sequence that includes the polymorphism are indicated. The genomic sequence identified in Table 3 may be accessed at the http address www.ncbi.nih.gov/entrez/query.fcgi, for example, by using the publicly available SNP reference number (e.g., rs1360457). The chromosome position refers to the position of the SNP within NCBI's Genome Build 34 and dbSNP 119, which may be accessed at the following http address: www.ncbi.nlm.nih.gov/mapview/map_search.cgi?chr=hum_chr.inf&query=. The “Contig Position” provided in Table 3 corresponds to a nucleotide position set forth in the contig sequence, and designates the polymorphic site corresponding to the SNP reference number. The sequence containing the polymorphisms also may be referenced by the “Sequence Identification” set forth in Table 3. The “Sequence Identification” corresponds to cDNA sequence that encodes associated target polypeptides (e.g., NRP1 of the invention. The position of the SNP within the cDNA sequence is provided in the “Sequence Position” column of Table 3. In the case of rs2034453, genetic evidence suggests an association with a region on chromosome 1 band p31.1 that includes two genes: fucose-1-phosphate guanylyltransferase (FPGT) (NM_{—}003838) and cardiac ankyrin repeat kinase (CARK) (NM_{—}015978). Also, the allelic variation at the polymorphic site and the allelic variant identified as associated with melanoma is specified in Table 3. All nucleotide sequences referenced and accessed by the parameters set forth in Table 3 are incorporated herein by reference.

Assay for Verifying, Allelotyping, and Genotyping SNPs

A MassARRAY™ system (Sequenom, Inc.) was utilized to perform SNP genotyping in a high-throughput fashion. This genotyping platform was complemented by a homogeneous, single-tube assay method (hME™ or homogeneous MassEXTEND™ (Sequenom, Inc.)) in which two genotyping primers anneal to and amplify a genomic target surrounding a polymorphic site of interest. A third primer (the MassEXTEND™ primer), which is complementary to the amplified target up to but not including the polymorphism, was then enzymatically extended one or a few bases through the polymorphic site and then terminated.

For each polymorphism, SpectroDESIGNER™ software (Sequenom, Inc.) was used to generate a set of PCR primers and a MassEXTEND™ primer which where used to genotype the polymorphism. Other primer design software could be used or one of ordinary skill in the art could manually design primers based on his or her knowledge of the relevant factors and considerations in designing such primers. Table 4 shows PCR primers and Table 5 shows extension primers used for analyzing the polymorphism set forth in Table 3. The initial PCR amplification reaction was performed in a 5 μl total volume containing 1×PCR buffer with 1.5 mM MgCl₂ (Qiagen), 200 μM each of dATP, dGTP, dCTP, dTTP (Gibco-BRL), 2.5 ng of genomic DNA, 0.1 units of HotStar DNA polymerase (Qiagen), and 200 nM each of forward and reverse PCR primers specific for the polymorphic region of interest.

TABLE 4

PCR Primers

SNP

| | | |
|---|---|---|
| Reference | Forward PCR primer | Reverse PCR primer |

| | | |
|---|---|---|
| rs1360457 | TTGTCCCAACTGAGGCTTTG | AACAGCCTTTCAGCTTTGGC |
| rs753050 | ACGTTGGATGCCTCTGTTTCCAACTCAAGG | ACGTTGGATGTTCACAGAGGTTACTAAGGG |
| rs729647 | AACCAACCCACTGGGTTGAC | TGATCTGGGAGAGTTTGGAG |
| rs8404 | GCCTCCTGTTGGGTCCTC | AAGGTATGGGGTGGGAGC |
| rs1044639 | CGCAAACAAAAAGGACACAC | CTCCTTTGTTTCCACCATCC |
| rs2034453 | TTGCTGGACAATAGAAAGAC | GTGACTGGAAACTGAGAATG |
| rs1904528 | GAAGACTGAAAAAAATCCACG | GCTATCTCTTTCACATTGCTC |

Samples were incubated at 95° C. for 15 minutes, followed by 45 cycles of 95° C. for 20 seconds, 56° C. for 30 seconds, and 72° C. for 1 minute, finishing with a 3 minute final extension at 72° C. Following amplification, shrimp alkaline phosphatase (SAP) (0.3 units in a 2 μl volume) (Amersham Pharmacia) was added to each reaction (total reaction volume was 7 μl) to remove any residual dNTPs that were not consumed in the PCR step. Samples were incubated for 20 minutes at 37° C., followed by 5 minutes at 85° C. to denature the SAP.

Once the SAP reaction was complete, a primer extension reaction was initiated by adding a polymorphism-specific MassEXTEND™ primer cocktail to each sample. Each MassEXTEND™ cocktail included a specific combination of dideoxynucleotides (ddNTPs) and deoxynucleotides (dNTPs) used to distinguish polymorphic alleles from one another. Methods for verifying, allelotyping and genotyping SNPs are disclosed, for example, in U.S. Pat. No. 6,258,538, the content of which is hereby incorporated by reference. In Table 5, ddNTPs are shown and the fourth nucleotide not shown is the dNTP.

TABLE 5

Extension Primers

| | |
|---|---|
| SNP | Termination |

| | | |
|---|---|---|
| Reference | Extend Probe | Mix |

| | | |
|---|---|---|
| rs1360457 | CAGCTTTGGCCAGGAGATG | ACT |
| rs753050 | AGCACATAACACAGCATGGC | ACT |
| rs729647 | GGTTGACGTCAACACAGGC | ACT |
| rs8404 | CGAGACTACCAGGAGAGCCC | ACG |
| rs1044639 | CATCCATCCAACCTGGCTC | CGT |
| rs2034453 | AAACTGAGAATGTTGATGGACA | ACT |
| rs1904528 | TCACATTGCTCTGCACTTTTG | ACG |

The MassEXTEND™ reaction was performed in a total volume of 9 μl, with the addition of 1× ThermoSequenase buffer, 0.576 units of ThermoSequenase (Amersham Pharmacia), 600 nM MassEXTEND™ primer, 2 mM of ddATP and/or ddCTP and/or ddGTP and/or ddTTP, and 2 mM of dATP or dCTP or dGTP or dTTP. The deoxy nucleotide (dNTP) used in the assay normally was complementary to the nucleotide at the polymorphic site in the amplicon. Samples were incubated at 94° C. for 2 minutes, followed by 55 cycles of 5 seconds at 94° C., 5 seconds at 52° C., and 5 seconds at 72° C.

Following incubation, samples were desalted by adding 16 μl of water (total reaction volume was 25 μl), 3 mg of SpectroCLEAN™ sample cleaning beads (Sequenom, Inc.) and allowed to incubate for 3 minutes with rotation. Samples were then robotically dispensed using a piezoelectric dispensing device (SpectroJET™ (Sequenom, Inc.)) onto either 96-spot or 384-spot silicon chips containing a matrix that crystallized each sample (SpectroCHIP™ (Sequenom, Inc.)). Subsequently, MALDI-TOF mass spectrometry (Biflex and Autoflex MALDI-TOF mass spectrometers (Bruker Daltonics) can be used) and SpectroTYPER RT™ software (Sequenom, Inc.) were used to analyze and interpret the SNP genotype for each sample.

Genetic Analysis

The minor allelic frequencies for the polymorphisms set forth in Table 3 were verified as being 10% or greater using the extension assay described above in a group of samples isolated from 92 individuals originating from the state of Utah in the United States, Venezuela and France (Coriell cell repositories).

Genotyping results for the allelic variant set forth in Table 3 are shown for female pools in Table 6 and for male pools in Table 8. In Table 6, “F case” and “F control” refer to female case and female control groups, and in Table 7, “M case” and “M control” refer to male case and male control groups.

TABLE 6
Female Genotyping Results

| SNP | | | p- | Odds |
|---|---|---|---|---|
| Reference | F case | F control | value | Ratio |
| rs1360457 | C = 0.869 | C = 0.903 | 0.089 | 0.71 |
| | T = 0.131 | T = 0.097 | | |
| rs753050 | A = 0.270 | A = 0.210 | 0.021 | 0.71 |
| | G = 0.730 | G = 0.790 | | |
| rs729647 | A = 0.578 | A = 0.620 | 0.187 | 1.19 |
| | G = 0.422 | G = 0.380 | | |
| rs8404 | C = 0.144 | C = 0.094 | 0.016 | 0.62 |
| | T = 0.856 | T = 0.906 | | |
| rs1044639 | A = 0.614 | A = 0.528 | 0.006 | 1.42 |
| | C = 0.386 | C = 0.472 | | |
| rs2034453 | G = 0.854 | G = 0.901 | 0.023 | 0.64 |
| | A = 0.146 | A = 0.099 | | |
| rs1904528 | T = 0.771 | T = 0.838 | 0.008 | 0.65 |
| | C = 0.229 | C = 0.162 | | |

TABLE 7
Male Genotyping Results

| SNP | | | p- | Odds |
|---|---|---|---|---|
| Reference | M case | M control | value | Ratio |
| rs1360457 | C = 0.876 | C = 0.917 | 0.042 | 0.66 |
| | T = 0.124 | T = 0.083 | | |
| rs753050 | A = 0.270 | A = 0.190 | 0.009 | 0.66 |
| | G = 0.730 | G = 0.810 | | |
| rs729647 | A = 0.543 | A = 0.640 | 0.004 | 1.48 |
| | G = 0.457 | G = 0.360 | | |
| rs8404 | C = 0.136 | C = 0.098 | 0.071 | 0.69 |
| | T = 0.864 | T = 0.902 | | |
| rs1044639 | A = 0.611 | A = 0.551 | 0.072 | 1.28 |
| | C = 0.389 | C = 0.449 | | |
| rs2034453 | G = 0.896 | G = 0.890 | 0.767 | 1.07 |
| | A = 0.104 | A = 0.110 | | |
| rs1904528 | T = 0.790 | T = 0.822 | 0.396 | 0.82 |
| | C = 0.210 | C = 0.178 | | |

The single marker alleles set forth in Table 3 were considered validated, since the genotyping data for the females, males or both pools were significantly associated with melanoma, and because the genotyping results agreed with the original allelotyping results. Particularly significant associations with melanoma are indicated by a calculated p-value of less than 0.05 for genotype results, which are set forth in bold text.

The odds ratios were calculated for the alleles at each SNP and the results are shown in Tables 6 and 7. An odds ratio is an unbiased estimate of relative risk which can be obtained from most case-control studies. Relative risk (RR) is an estimate of the likelihood of disease in the exposed group (susceptibility allele or genotype carriers) compared to the unexposed group (not carriers). It can be calculated by the following equation:
RR═I_{A}/Iₐ
I_{A} is the incidence of disease in the A carriers and Iₐ is the incidence of disease in the non-carriers.
RR>1 indicates the A allele increases disease susceptibility.
RR<1 indicates the a allele increases disease susceptibility.
   For example, RR=1.5 indicates that carriers of the A allele have 1.5 times the risk of disease than non-carriers, i.e., 50% more likely to get the disease.

Case-control studies do not allow the direct estimation of I_{A} and Iₐ, therefore relative risk cannot be directly estimated. However, the odds ratio (OR) can be calculated using the following equation:
OR=(n_{DA}n_{da})/(n_{dA}n_{Da})=p_{DA}(1−p_{dA})/p_{dA}(1−p_{DA}), or
OR=((case f)/(1−case f))/((control f)/(1−control f)), where f=susceptibility allele frequency.

An odds ratio can be interpreted in the same way a relative risk is interpreted and can be directly estimated using the data from case-control studies, i.e., case and control allele frequencies. The higher the odds ratio value, the larger the effect that particular allele has on the development of melanoma, thus possessing that particular allele translates to having a higher risk of developing melanoma.

NID2 Non-Synonomous SNP

In addition, a non-synonomous, coding SNP (rs3742536) located at amino acid position 656 (P656S) of NID2 was genotyped and found to be significantly associated with the occurrence of melanoma. This SNP (rs3742536) was genotyped as described in Examples 1 and 2 herein using the following PCR primers, extend primer and termination mix: forward primer—ACGTTGGATGAGACCAACATTCAAGGCCAG; reverse primer—ACGTTGGATGAGTGGTACAGCTCCTTGTAG; extend primer—GGCTGTGAAATTTGCTG; and termination mix-ACT. The genotyping results for female (F) and male (M) cases and controls are shown in Table 8.

TABLE 8
Genotpying Results

| | Chromosome | | | | | | Odds |
|---|---|---|---|---|---|---|---|
| Rs number | Position | Sex | Alleles | Case AF | Control AF | p-Value | Ratio |
| rs3742536 | 50497467* | Male | C/T | T = 0.261 | T = 0.192 | 0.0144 | 0.67 |
| | | | | C = 0.739 | C = 0.808 | | |
| | | Female | | T = 0.257 | T = 0.205 | 0.0561 | 0.75 |
| | | | | C = 0.743 | C = 0.795 | | |

*based on NCBI's Build 34

### Example 3

Samples and Pooling Strategies for the Replication Cohort

The single marker alleles set forth in Table 3 were genotyped again in a replication cohort to further validate their association with melanoma. Like the original study population described in Examples 1 and 2, the replication cohort consisted of individuals diagnosed with melanoma (cases) and individuals free of melanoma (controls). The case and control samples were selected and genotyped as described below.

Sample Selection

Blood samples were collected from individuals diagnosed with melanoma, which were referred to case samples. Also, blood samples were collected from individuals not diagnosed with me lanoma or a history of melanoma; these samples served as gender and age-matched controls.

DNA Extraction from Blood Samples

Blood samples for DNA preparation were taken in 5 EDTA tubes. If it was not possible to get a blood sample from a patient, a sample from the cheek mucosa was taken. Red blood cells were lysed to facilitate their separation from the white blood cells. The white cells were pelleted and lysed to release the DNA. Lysis was done in the presence of a DNA preservative using an anionic detergent to solubilize the cellular components. Contaminating RNA was removed by treatment with an RNA digesting enzyme. Cytoplasmic and nuclear proteins were removed by salt precipitation.

Genomic DNA was then isolated by precipitation with alcohol (2-propanol and then ethanol) and rehydrated in water. The DNA was transferred to 2-ml tubes and stored at 4° C. for short-term storage and at −70° C. for long-term storage.

Pooling Strategies

Samples were placed into one of four groups based on disease status. The four groups were female case samples, female control samples, male case samples, and male control samples. A select set of samples from each group were utilized to generate pools, and one pool was created for each group.

Replication samples were obtained from QIMR (Queensland Institute of Medical Research) through Nick Martin. All samples are of Australian descent. Sample sources were as follows: a.) Queensland Familial Melanoma Study-702 cutaneous malignant melanoma cases plus 46 unaffected relatives; and b.) Twin mole study-2367 controls, consisting of adolescent twins and siblings closest in age that had nevi counted and other skin and pigmentary phenotypes assessed. The subjects available for replication from Australia included 702 mostly unrelated melanoma cases from the Queensland Familial Melanoma study and 2367 controls from the Twin Mole study with subjects organized into pedigrees with up to eight unaffected individuals.

To facilitate a direct comparison with the discovery study design and subsequent meta analyses, we selected a subset of the Australian sample to produce a research data set of unrelated cases and controls. For cases, this was done by selecting the proband from all families within the familial melanoma study. For controls, this was accomplished by selecting the pedigree founders from the twin study, usually the father and mother of the collected twins. The resulting data set consisted of 376 female and 300 male cases, and 640 female and 515 male controls.

### Example 4

### Association of Polymorphic Variants with Melanoma in the Replication Cohort

The associated SNPs from the initial scan were re-validated by genotyping the associated SNP across the replication cohort described in Example 3. The results of the genotyping were then analyzed, allele frequencies for each group were calculated from the individual genotyping results, and a p-value was calculated to determine whether the case and control groups had statistically significantly differences in allele frequencies for a particular SNP. The replication genotyping results were considered significant with a calculated p-value of less than 0.05 for genotype results, which are set forth in bold text. See Tables 8 and 9 herein.

Assay for Verifying Allelotyping, and Genotyping SNPs

Genotyping of the replication cohort was performed using the same methods used for the original genotyping, as described herein. A MassARRAY™ system (Sequenom, Inc.) was utilized to perform SNP genotyping in a high-throughput fashion. This genotyping platform was complemented by a homogeneous, single-tube assay method (hME™ or homogeneous MassEXTEND™ (Sequenom, Inc.)) in which two genotyping primers anneal to and amplify a genomic target surrounding a polymorphic site of interest. A third primer (the MassEXTEND™ primer), which is complementary to the amplified target up to but not including the polymorphism, was then enzymatically extended one or a few bases through the polymorphic site and then terminated.

For each polymorphism, SpectroDESIGNER™ software (Sequenom, Inc.) was used to generate a set of PCR primers and a MassEXTEND™ primer which where used to genotype the polymorphism. Other primer design software could be used or one of ordinary skill in the art could manually design primers based on his or her knowledge of the relevant factors and considerations in designing such primers. Table 4 shows PCR primers and Table 5 shows extension probes used for analyzing (e.g., genotyping) polymorphisms. The initial PCR amplification reaction was performed in a 5 μl total volume containing 1×PCR buffer with 1.5 mM MgCl₂ (Qiagen), 200 μM each of dATP, dGTP, dCTP, dTTP (Gibco-BRL), 2.5 ng of genomic DNA, 0.1 units of HotStar DNA polymerase (Qiagen), and 200 nM each of forward and reverse PCR primers specific for the polymorphic region of interest.

Samples were incubated at 95° C. for 15 minutes, followed by 45 cycles of 95° C. for 20 seconds, 56° C. for 30 seconds, and 72° C. for 1 minute, finishing with a 3 minute final extension at 72° C. Following amplification, shrimp alkaline phosphatase (SAP) (0.3 units in a 2 μl volume) (Amersham Pharmacia) was added to each reaction (total reaction volume was 7 μl) to remove any residual dNTPs that were not consumed in the PCR step. Samples were incubated for 20 minutes at 37° C., followed by 5 minutes at 85° C. to denature the SAP.

Once the SAP reaction was complete, a primer extension reaction was initiated by adding a polymorphism-specific MassEXTEND™ primer cocktail to each sample. Each MassEXTEND™ cocktail included a specific combination of dideoxynucleotides (ddNTPs) and deoxynucleotides (dNTPs) used to distinguish polymorphic alleles from one another. Methods for verifying, allelotyping and genotyping SNPs are disclosed, for example, in U.S. Pat. No. 6,258,538, the content of which is hereby incorporated by reference. In Table 5, ddNTPs are shown and the fourth nucleotide not shown is the dNTP.

The MassEXTEND™ reaction was performed in a total volume of 9 μl, with the addition of 1× ThermoSequenase buffer, 0.576 units of ThermoSequenase (Amersham Pharmacia), 600 nM MassEXTEND™ primer, 2 mM of ddATP and/or ddCTP and/or ddGTP and/or ddTTP, and 2 mM of dATP or dCTP or dGTP or dTTP. The deoxy nucleotide (dNTP) used in the assay normally was complementary to the nucleotide at the polymorphic site in the amplicon. Samples were incubated at 94° C. for 2 minutes, followed by 55 cycles of 5 seconds at 94° C., 5 seconds at 52° C., and 5 seconds at 72° C.

Following incubation, samples were desalted by adding 16 μl of water (total reaction volume was 25 μl), 3 mg of SpectroCLEAN™ sample cleaning beads (Sequenom, Inc.) and allowed to incubate for 3 minutes with rotation. Samples were then robotically dispensed using a piezoelectric dispensing device (SpectroJET™ (Sequenom, Inc.)) onto either 96-spot or 384-spot silicon chips containing a matrix that crystallized each sample (SpectroCHIP™ (Sequenom, Inc.)). Subsequently, MALDI-TOF mass spectrometry (Biflex and Autoflex MALDI-TOF mass spectrometers (Bruker Daltonics) can be used) and SpectroTYPER RT™ software (Sequenom, Inc.) were used to analyze and interpret the SNP genotype for each sample.

Genetic Analysis

The minor allelic frequencies for the polymorphisms set forth in Table 3 were verified as being 10% or greater using the extension assay described above in a group of samples isolated from 92 individuals originating from the state of Utah in the United States, Venezuela and France (Coriell cell repositories).

Replication genotyping results are shown for females and males in Table 9. P-values and odds ratios are provided in Table 10.

TABLE 9
Female and Male Replication Genotyping Results

| | | Allele | | | Allele | | |
|---|---|---|---|---|---|---|---|
| | | frequency-females | | | frequency-males | | |
| dbSNP | | | Con- | | | Con- | |
| rs# | Gene | Case | trol | Delta | Case | trol | Delta |
| rs8404 | CDK10 | 0.87 | 0.91 | −0.043 | 0.88 | 0.91 | −0.029 |
| rs1044639 | PCLO | 0.60 | 0.57 | 0.031 | 0.61 | 0.56 | 0.042 |
| rs2034453 | FPGT/ | 0.85 | 87.00 | −0.022 | 0.84 | 0.87 | −0.026 |
| | TNNI3K | | | | | | |
| | (CARK) | | | | | | |
| rs1904528 | REPS2 | 0.78 | 0.79 | −0.005 | 0.77 | 0.82 | −0.051 |

TABLE 10
Female and Male Replication Genotyping Analysis

| | | Replication F | | Replication M | | Summary | |
|---|---|---|---|---|---|---|---|
| dbSNP | | p- | | p- | | p- | |
| rs# | Gene | value | OR | value | OR | value | OR |
| rs8404 | CDK10 | 0.002 | 0.65 | 0.054 | 0.72 | 0.000004 | 0.67 |
| rs1044639 | PCLO | 0.180 | 1.13 | 0.100 | 1.19 | 0.000290 | 1.23 |
| rs2034453 | FPGT/ | 0.170 | 0.83 | 0.150 | 0.81 | 0.014000 | 0.82 |
| | TNNI3K | | | | | | |
| | (CARK) | | | | | | |
| rs1904528 | REPS2 | 0.800 | 0.97 | 0.076 | 0.73 | 0.033000 | 0.80 |

Meta analyses, combining the results of the German discovery sample and the Australian replication sample, were carried out using a random effects (DerSimonian-Laird) procedure. Statistically significant associations with melanoma are indicated by a calculated p-value of less than 0.05 for genotype results, which are set forth in bold text.

The absence of a statistically significant association in the replication cohort should not be interpreted as minimizing the value of the original finding. There are many reasons why a biologically derived association identified in a sample from one population would not replicate in a sample from another population. The most important reason is differences in population history. Due to bottlenecks and founder effects, there may be common disease predisposing alleles present in one population that are relatively rare in another, leading to a lack of association in the candidate region. Also, because common diseases such as melanoma are the result of susceptibilities in many genes and many environmental risk factors, differences in population-specific genetic and environmental backgrounds could mask the effects of a biologically relevant allele.

### Example 5

### NRP1 Proximal SNPs

It has been discovered that a polymorphic variation in a gene encoding the receptor neuropilin 1 (NRP1 is associated with the occurrence of melanoma.

Ninety-five allelic variants located within or nearby the target gene were identified and subsequently allelotyped in melanoma case and control sample sets as described in Examples 1 and 2. The polymorphic variants are set forth in Table 11. The chromosome position provided in column four of Table 11 is based on Genome “Build 34” of NCBI's GenBank and dbSNP 119.

TABLE 11

| | | | | | Allele | |
|---|---|---|---|---|---|---|
| | | | Position | | Present | |
| | Chromo- | Chromosome | in SEQ | Allele | in SEQ | IUPAC |
| dbSNP rs# | some | Position | ID NO. 1 | Variants | ID NO: 1 | Code |
| rs7910405 | 10 | 33590041 | 2,141 | T/C | T | Y |
| rs5784331 | 10 | 33590861 | 2,961 | —/A | A | N |
| rs6481845 | 10 | 33596683 | 8,783 | T/C | T | Y |
| rs2065364 | 10 | 33598008 | 10,108 | C/T | T | Y |
| rs10643659 | 10 | 33599549 | 11,649 | —/GTTT | G | N |
| rs7896887 | 10 | 33603031 | 15,131 | A/G | G | R |
| rs7086456 | 10 | 33606418 | 18,518 | C/T | C | Y |
| rs6481846 | 10 | 33606937 | 19,037 | G/A | G | R |
| rs7475391 | 10 | 33606955 | 19,055 | C/T | T | Y |
| rs7477281 | 10 | 33606956 | 19,056 | C/T | C | Y |
| rs7475393 | 10 | 33606959 | 19,059 | C/T | T | Y |
| rs7475394 | 10 | 33606966 | 19,066 | C/T | T | Y |
| rs7477282 | 10 | 33606970 | 19,070 | C/T | C | Y |
| rs7475396 | 10 | 33606978 | 19,078 | C/T | T | Y |
| rs2768420 | 10 | 33609638 | 21,738 | G/A | G | R |
| rs9418057 | 10 | 33612486 | 24,586 | C/T | T | Y |
| rs7070290 | 10 | 33615210 | 27,310 | G/A | G | R |
| rs1360457 | 10 | 33620935 | 33,035 | T/C | A | R |
| rs1411924 | 10 | 33623568 | 35,668 | G/A | C | Y |
| rs5784335 | 10 | 33626177 | 38,277 | T/— | T | N |
| rs2776925 | 10 | 33718511 | 130,611 | C/T | A | R |
| rs2776924 | 10 | 33718757 | 130,857 | G/C | T | S |
| rs1952983 | 10 | 33719174 | 131,274 | A/C | T | K |
| rs869636 | 10 | 33722278 | 134,378 | C/T | A | R |
| rs869637 | 10 | 33722444 | 134,544 | C/T | G | R |
| rs2804470 | 10 | 33723869 | 135,969 | G/A | G | R |
| rs2776928 | 10 | 33724644 | 136,744 | T/C | T | Y |
| rs999966 | 10 | 33724924 | 137,024 | C/T | A | R |
| rs2804475 | 10 | 33724926 | 137,026 | C/G | G | S |
| rs4934858 | 10 | 33728527 | 140,627 | C/T | C | Y |
| rs4934863 | 10 | 33729317 | 141,417 | C/T | T | Y |
| rs4934864 | 10 | 33729327 | 141,427 | C/T | G | R |
| rs1331326 | 10 | 33730129 | 142,229 | G/A | C | Y |
| rs1331325 | 10 | 33730408 | 142,508 | A/G | T | Y |
| rs4934871 | 10 | 33733328 | 145,428 | G/A | G | R |
| rs4277057 | 10 | 33736929 | 149,029 | G/T | T | K |
| rs2776930 | 10 | 33738120 | 150,220 | C/T | A | R |
| rs1331324 | 10 | 33739704 | 151,804 | G/C | G | S |
| rs2776932 | 10 | 33742645 | 154,745 | C/T | G | R |
| rs4934896 | 10 | 33743848 | 155,948 | A/G | A | R |
| rs2776933 | 10 | 33745084 | 157,184 | T/C | C | Y |
| rs4934901 | 10 | 33746285 | 158,385 | A/T | T | W |
| rs1015300 | 10 | 33747483 | 159,583 | G/C | A | S |
| rs2776934 | 10 | 33750081 | 162,181 | G/C | G | S |
| rs2776935 | 10 | 33750537 | 162,637 | G/A | G | R |
| rs2776936 | 10 | 33751120 | 163,220 | C/T | T | Y |
| rs2804491 | 10 | 33751126 | 163,226 | A/C | A | M |
| rs2804492 | 10 | 33752285 | 164,385 | T/C | T | Y |
| rs2804493 | 10 | 33755915 | 168,015 | A/G | G | R |
| rs2776937 | 10 | 33756428 | 168,528 | A/G | A | R |
| rs2804494 | 10 | 33759655 | 171,755 | G/C | T | S |
| rs4934914 | 10 | 33761218 | 173,318 | G/A | G | R |
| rs2776922 | 10 | 33762121 | 174,221 | T/C | C | Y |
| rs2804495 | 10 | 33762739 | 174,839 | G/T | C | M |
| rs2804496 | 10 | 33764040 | 176,140 | T/C | A | R |
| rs4934597 | 10 | 33764960 | 177,060 | T/C | T | Y |
| rs2804497 | 10 | 33766276 | 178,376 | G/T | A | M |
| rs2804498 | 10 | 33770946 | 183,046 | T/C | A | R |
| rs2026319 | 10 | 33771741 | 183,841 | G/C | C | S |
| rs1888684 | 10 | 33773216 | 185,316 | T/C | G | R |
| rs3750733 | 10 | 33773490 | 185,590 | A/G | C | Y |
| rs4934927 | 10 | 33778301 | 190,401 | G/C | G | S |
| rs2776946 | 10 | 33782804 | 194,904 | T/C | T | Y |
| rs4934599 | 10 | 33784201 | 196,301 | T/C | T | Y |
| rs4934934 | 10 | 33785140 | 197,240 | A/G | A | R |
| rs2776944 | 10 | 33785311 | 197,411 | T/G | T | K |
| rs2776943 | 10 | 33786453 | 198,553 | C/T | A | R |
| rs2804886 | 10 | 33787179 | 199,279 | G/A | G | R |
| rs2804900 | 10 | 33787641 | 199,741 | G/A | T | Y |
| rs2804902 | 10 | 33787672 | 199,772 | T/C | C | Y |
| rs2768397 | 10 | 33788724 | 200,824 | G/C | C | S |
| rs2768399 | 10 | 33788911 | 201,011 | A/G | A | R |
| rs2776941 | 10 | 33790186 | 202,286 | C/G | A | S |
| rs2804888 | 10 | 33791746 | 203,846 | G/A | A | R |
| rs2804499 | 10 | 33791760 | 203,860 | C/G | T | S |
| rs2804889 | 10 | 33792103 | 204,203 | G/A | G | R |
| rs2804891 | 10 | 33792647 | 204,747 | G/A | C | Y |
| rs2776940 | 10 | 33794228 | 206,328 | A/T | A | W |
| rs2804897 | 10 | 33794781 | 206,881 | C/T | C | Y |
| rs2804896 | 10 | 33795259 | 207,359 | T/C | C | Y |
| rs2776939 | 10 | 33795324 | 207,424 | C/T | C | Y |
| rs2804894 | 10 | 33797330 | 209,430 | T/C | C | Y |
| rs2804501 | 10 | 33798562 | 210,662 | A/T | C | W |
| rs2183715 | 10 | 33799002 | 211,102 | A/C | T | K |
| rs2254822 | 10 | 33799071 | 211,171 | G/A | T | Y |
| rs2150686 | 10 | 33799262 | 211,362 | G/A | A | R |
| rs2804893 | 10 | 33799778 | 211,878 | T/C | A | R |
| rs2804502 | 10 | 33800099 | 212,199 | T/C | A | R |
| rs2804892 | 10 | 33800318 | 212,418 | T/C | C | Y |
| rs2183714 | 10 | 33803309 | 215,409 | G/A | T | Y |
| rs2804503 | 10 | 33804955 | 217,055 | C/T | T | Y |
| rs1320485 | 10 | 33806274 | 218,374 | T/C | G | R |
| rs4934603 | 10 | 33808882 | 220,982 | A/G | T | Y |
| rs2804890 | 10 | 33808922 | 221,022 | C/T | A | R |
| rs1854145 | 10 | 33809260 | 221,360 | A/G | G | R |

Assay for Verifying and Allelotyping SNPs

The methods used to verify and allelotype the seventy-six proximal SNPs of Table 11 are the same methods described in Examples 1 and 2 herein. The primers used in these assays are provided in Table 12 and Table 13, respectively.

TABLE 12

| | | |
|---|---|---|
| dbSNP rs # | Forward PCR Primer | Reverse PCR Primer |

| | | |
|---|---|---|
| rs6481844 | GGAGAAAACCAAGATGGCAC | CATAATTGGCAACTCCCCAG |
| rs7910405 | GAGTCAGGACTTGCAAACAG | CAGACTGAGAACCAACTTAG |
| rs5784331 | GAATTGGGAGGCATGCTTTG | AGCAGATTTACTCCCTTCCC |
| rs6481845 | AATGCCATCAACTTGGGCTC | CAGCAGCTACCAAGCAGTAT |
| rs2065364 | AATCTTGCTGGTGCTCACTC | CAGAAGGAGACACTGTCTCA |
| rs10643659 | TTATTACGTAAGTTCCTTCC | CAGTCTACCTCTGAAAATAG |
| rs7896887 | CCTCTCCCACCACTGTTTTA | AAGTGCGGTGATAGGAAAGC |
| rs7086456 | CAGTCTACCTCTGAAAATAG | TTATTACGTAAGTTCCTTCC |
| rs6481846 | ATTGGAATCCCTCCTGGATC | TTTTGAACAGGGCACCACAG |
| rs747539 | TTTAGGAATGGAAATCCTGGG | CCTGGTTGTCAGGCTTTAAA |
| rs747728 | TTTATTACGTAAGTTCCTTCC | CAGTCTACCTCTGAAAATAG |
| rs7475393 | AAATAGCTCATGTACCCCAG | CCTGCCATAGAGCATATTAC |
| rs7475394 | TTATTACGTAAGTTCCTTCC | CAGTCTACCTCTGAAAATAG |
| rs7477282 | TTATTACGTAAGTTCCTTCC | CAGTCTACCTCTGAAAATAG |
| rs7475396 | TTATTACGTAAGTTCCTTCC | CAGTCTACCTCTGAAAATAG |
| rs2768420 | TTTCCTTGACAGATAACTGC | GCAGCTAAAAGACATCATTG |
| rs9418057 | TTATTACGTAAGTTCCTTCC | CAGTCTACCTCTGAAAATAG |
| rs7070290 | GTGGCACTAGTTTTCAGAAG | GCCCTATGAAAACCAGATTC |
| rs1360457 | CAGGATTATCTGACAGGAGG | TATTTTCCTTTCCCACCAGC |
| rs1411924 | TCCTTCCTCTCCTCCTTCTC | CCTACTTTTCCTTCAAACAC |
| rs5784335 | TGATTCACATTCTCCTCCCC | TAGGCATGAGCACTTTGTGG |
| rs2776925 | ACCACTCATGTCTGCCTGCT | TGAAGGATCTTTACTCAGGC |
| rs2776924 | CCCCAAGAATCTTGCACTTC | AGGTCTTCAAGACCTGTTCC |
| rs1952983 | GCTGGCACTCATTTTCTCTC | AGTTCCACATAGCTGGAGAG |
| rs869636 | CACACTCACTGAAGCGTTTG | CCAAATAACATGCTGATCGC |
| rs869637 | AAGTGTTCTTCTCCCACAGC | CCTTTAACCCTTACAGAGCC |
| rs2804470 | GGTGCAGCTAAAGAGAAACG | TGGCCGAGGGTTAATTTTTG |
| rs2776928 | GACAGAAAGATTTGTGACGC | CATTTGACAGCTGAGATCCC |
| rs999966 | GCAGTGGGCACTAACCTAAG | GGTTGCGGTTATCTTAGCAG |
| rs2804475 | TGTGGCATCATTTGTCTTCC | CATCATTCCCCCAGTTCTAG |
| rs4934858 | GATCATTGGTGGAAACAGGG | CCACCCATCATTACAATCGC |
| rs4934863 | GCTTCTATTAGGGTGTTGGC | CAGTGCTCACTCAGTTAGTC |
| rs4934864 | AGTCTCACCAAGACCTTCTG | CACCTAGGGATTTATAGACG |
| rs1331326 | TTTAAACATGGTCTCGTGGC | CTACAGTGGCAAAGAACACG |
| rs1331325 | CCTTCTTCATTCATCAGGGC | AAAACTGCCATTCCTCCCAC |
| rs4934871 | GGAATATAAGTCCTGCAGCC | TGCACATATAGACGTATCAC |
| rs4277057 | ACACCCTGCATTTTCTCTTG | GCAAATGTGCTAAGGCATCG |
| rs2776930 | CCTCTTCCAAAAGTAAGCCC | GGAAGAAACCAGTCCAAGAG |
| rs1331324 | GTGCTTATTACCCGAGTGAC | CCTGTACGTGTACAGGTTTG |
| rs2776932 | CTGCAAGAACCTGAACTCTG | AGAAATCGAGGTGTTGGCTG |
| rs4934896 | ACAGATATGCTGCAGTGACC | ATTACCCAAGTAGGCTTCCC |
| rs2776933 | CTGAGTATCTGGGACTACAG | TGGTTAACACGGTGAAACCC |
| rs4934901 | GCACTAGTCCAGCATTTTCC | ACTTTGCTTCATACCATGGA |
| rs1015300 | CCTCTTCTGTAATCTCTAGTG | AACGGACAAGTACTCTAAGC |
| rs2776934 | CTCCCTTTCTTGAGGTTGAC | CAAACACGAATCTAGGAGCC |
| rs2776935 | AATACTCTCACCACCACCAC | CCCAGCACAAATAAGTGCTC |
| rs2776936 | CAATCCCAGGGGATTATAGC | TGTTTACCGATGCTCGTACC |
| rs2804491 | AACTCCTGACCTCAAGTGAC | AAATGAACTCCTCTTGGCCG |
| rs2804492 | ATGCCTGAAATCCCAGCTAC | AGTGGCTCAATCTTGGCTCC |
| rs2804493 | TCAGCTAATTCCCAGCATTC | GGAGTTGACAGTTCAAATCCT |
| rs2776937 | GAAATCCTGTGGTAGGAATC | GACATCTCATTTTCTAGGTG |
| rs2804494 | CCTAGACCTTAGTCTTACCC | TCAGAAGACCACATTCTCAG |
| rs4934914 | TAAGTCCTGCAGCCACTTTG | GCATACACAAATATATGCAC |
| rs2776922 | TTGAAATCCTTTCCACTGGC | GTTCTGTTGCCTTCATGCTC |
| rs2804495 | GGAAGATCTTTGAGAGTTAGC | AGGAACAGCCACATTTTGAC |
| rs2804496 | AAAGAAGAAGGCCCCAACAC | TTCTTGGCCCTGATTTCTCC |
| rs4934597 | TTCTTGACCTCGTGATCCAC | AATTAACCCTCGGCCAGGTG |
| rs2804497 | AACTCCCTCCTTCCCTTAAC | CTCAGGGTGTATTTTACACG |
| rs2804498 | AGCACTTAAGACAGCCACTC | GTGGCCAGATTCTCTGAATG |
| rs2026319 | AATTGGCATGCTGATCTGGG | GGGCTTTTCCTTACCTCTGT |
| rs1888684 | AACAGCCTTTCAGCTTTGGC | TTGTCCCAACTGAGGCTTTG |
| rs3750733 | CTGCCTGTCACTTACCGTTG | GCTCCTCTGCGCCGTGCTC |
| rs4934927 | TTTATTCATGGCAGAAGGCG | ATAAAGTGCAGTACCTCCCC |
| rs2776946 | CCCATAAAGGAAGCAGAAGC | TTTGTCCCCTATCCTCTCTC |
| rs4934599 | TTTTCGAGACGGAGTCTTGC | GAGGCGGAGATTACAGTAAG |
| rs4934934 | GGATGAAATGAGAATGACCC | GAGGAGTCGACTGTTGAATG |
| rs2776944 | CATAAGGCCATTCATGAGGG | GAAAGAGCTTGGGATCATGG |
| rs2776943 | GGAGTTGACAGTTCAAATCCT | TCAGCTAATTCCCAGCATTC |
| rs2804886 | GAAAAGAACATTATTTGCAGC | CTCCCTTTTTCTTATTAGTC |
| rs2804900 | CTAATTTCACCTGTAGGATG | TTCACATGGTGAAAGCAGGG |
| rs2804902 | AAGTTTGAGAACCACTGGGC | CATGAATCCCTCTGCAACTC |
| rs2768397 | CACACCAGAGCTCTTTAATC | GAGAGATTGAACAGAGCCAG |
| rs2768399 | CAAGATGAGATTTGGGTGGG | TGGACTGGTTTCTTCCTCAC |
| rs277694 | TTGGTTTATGGTCTTGGCGTC | GGGAAGAAAAGACGACCTAC |
| rs2804888 | TAACATGTCCACAGCCTTCC | GGATGGTTTCACTACTGTATG |
| rs2804499 | TGGCTCACTCGCAAACATTG | ATGTTGGCCAGGCTGGTTTC |
| rs2804889 | ACACCCAACTCTCTATGAGC | AGGTCCTGACCATGCATGAG |
| rs2804891 | GTGAACAAAAGTAGACTCCC | GCACTCTCTGGGATAATTTC |
| rs2776940 | TACATGAAGGCAATCGCACC | TGGTGGTGGATAAGCCAATG |
| rs2804897 | ATCTGGTCAGGTTTGGTCTC | CAAATAGAACTGACATCTGC |
| rs2804896 | AAGACCCTCTTCCTGGTTTG | TGCTTTCTCTCTCTTCTCCC |
| rs2776939 | TTTCCAGCTTGCAGAACCTC | TAGAGCATGATCTAGGTCCC |
| rs2804894 | CATTAATATGTGGCCATGGG | AGGGGTACACAATTCAATCC |
| rs2804501 | AGTTCGTTGCACCTTGAGAG | GACACTACTAACTGCCCTAG |
| rs2183715 | ATGGATTGAGCCACACTCTC | TGTCTCTAGACGTGTGTTGG |
| rs2254822 | TTCAGTGAAAGTTGCCTGTG | AGAGGTGCAATAACTGGTGG |
| rs2150686 | TTCAGTGAAAGTTGCCTGTG | AGAGGTGCAATAACTGGTGG |
| rs2804893 | ATACAAAAATTAGCCGGGCG | CTCCTGGGTTCAAGAGATTC |
| rs2804502 | AATGTGGCAGCATTTCAGGC | CTGGGCTGCCTATATTGTTC |
| rs2804892 | TAATTTCCTCCATCCCTCCC | CCCATGAAAGACTTAACCTTC |
| rs2183714 | TTTAAACATGGTCTCGTGGC | CTACAGTGGCAAAGAACACG |
| rs2804503 | GTCCTCTCTCTGTTTTAGGC | AATCTGTTTGCTTACTTTTC |
| rs1320485 | ATGCCTGAAATCCCAGCTAC | AGTGGCTCAATCTTGGCTCC |
| rs4934603 | CTCTCCTCTGTGTAACCAAC | GTTACAAGTATGCGATCTTCC |
| rs2804890 | ACTTAAGTGGCATCAGGTAG | ATCACAAATTGGAAGCCAAC |
| rs1854145 | GGATCCTAATTTGCGCTGAC | TCTGGGCAGTAAGTTTCAGG |

TABLE 13

Term

| | | |
|---|---|---|
| dbSNP rs # | Extend Primer | Mix |

| | | |
|---|---|---|
| rs6481844 | AACAACTGGAAAACCTTGGCT | ACT |
| rs7910405 | GATGTTCAGTGCTTGCTTTGC | ACT |
| rs5784331 | ATGATTCCAGGGGATTTTTTTTT | ACG |
| rs6481845 | AGAAGGTGAATGCTATAACTAAA | ACT |
| rs2065364 | TCTCCTCCCCATGTGCT | ACG |
| rs10643659 | CATATTATTTCAGAAATTATGGTTG | ACT |
| rs7896887 | CTGGGACTACAGACGCCC | ACT |
| rs7086456 | TCATGTACCCCAGTAATGTATA | ACG |
| rs6481846 | GAGAGAAGGAAGGAAGGAAAA | ACG |
| rs7475391 | TTCGTTTTCCTTCCTTCCTTC | ACG |
| rs7477281 | TCGTTTTCCTTCCTTCCTTCT | ACG |
| rs7475393 | TTTTCCTTCCTTCCTTCTCTC | ACG |
| rs7475394 | CCTTCCTTCTCTCTCTCTCT | ACG |
| rs7477282 | TCCTTCTCTCTCTCTCTTTCT | ACG |
| rs7475396 | TCTCTCTCTTTCTCTCTCTCT | ACG |
| rs2768420 | CACCATGAGATGCAGGA | ACG |
| rs9418057 | TGGTGCTCACTCTTTGCGG | ACG |
| rs7070290 | GATGGAGTTTAAAAGCACAAAG | ACG |
| rs1360457 | CAGCTTTGGCCAGGAGATG | ACT |
| rs1411924 | ATTTATAATCTACATTGTACACAC | ACG |
| rs5784335 | TTCCATGTTCATAATTTTTTTTTT | ACT |
| rs2776925 | CCAGTGGTGTGAGCTTC | ACG |
| rs2776924 | GAGGTTGACTCATGATTT | ACT |
| rs1952983 | CCTGATTGAAGCAAAACCAGAA | ACT |
| rs869636 | TGTTCAACAGACCCTTC | ACG |
| rs869637 | GTAATCTCTAGTGTTTATTGAAAG | ACG |
| rs2804470 | TTGCAGCATTTCCCTAA | ACG |
| rs2776928 | GTGGATCCTAATGATCAC | ACT |
| rs999966 | GGAGAACTGCTTGAACC | ACG |
| rs2804475 | AGAACTGCTTGAACCCG | ACT |
| rs4934858 | TGGGATTTCGTTACCTAA | ACG |
| rs4934863 | CAACCTGAGGATGTATAGAA | ACG |
| rs4934864 | GGATGTATAGAATGCCTATATG | ACG |
| rs1331326 | TTCCTTGATCTCTTTCTTC | ACG |
| rs1331325 | TTGCGCTGACAAAAACC | ACT |
| rs4934871 | GAGATATGTAGGACCCC | ACG |
| rs4277057 | AGAGGGTAGTAAATCCTG | CGT |
| rs2776930 | TGCTATTACTACTTCTACCTAC | ACG |
| rs1331324 | GTGTGTGCACATCTCAA | ACT |
| rs2776932 | CGTAGTTCTTCGCCCCT | ACG |
| rs4934896 | ACCCCATCTAAACAAAAG | ACT |
| rs2776933 | TAGCCGGGCGTGGTGGT | ACT |
| rs4934901 | TGGGATCATGAATCCTAG | CGT |
| rs1015300 | CTAAACCCTTGTGACATG | ACT |
| rs2776934 | TTTTACTGACTCTTCAAGTG | ACT |
| rs2776935 | TATCTTCATCTGCAGCC | ACG |
| rs2776936 | GACAGTTCAAATCCTTTTTTT | ACG |
| rs280449 | TTGTCTAGTATTTCTAAACACTG | ACT |
| rs2804492 | CTTAGTCTTACCCATGAAC | ACT |
| rs2804493 | GGAGTTGCCAATTATGTATAAG | ACT |
| rs2776937 | GGTGGGGATAAGGAGCT | ACT |
| rs2804494 | GGGTGTTGGAGTGGGTG | ACT |
| rs4934914 | CCCTCATTCATACAGTTT | ACG |
| rs2776922 | TTTGTGACGCAGCAACT | ACT |
| rs2804495 | GGTTGATTGGCCTGTAT | CGT |
| rs2804496 | AGGCAGGCATCACTTGA | ACT |
| rs4934597 | TTTTTCCTAACTCAGTGC | ACT |
| rs2804497 | GTATAGTCAACTTCCACC | CGT |
| rs2804498 | TCCATCATAAAATGTGACAAT | ACT |
| rs2026319 | TGACTTCTGAGAAAGTGA | ACT |
| rs1888684 | ATGCTGATCTGGGAAGGTAG | ACT |
| rs3750733 | TTACCGTTGCGAAAAGC | ACT |
| rs4934927 | TGAATCTTTGGTAACACC | ACT |
| rs2776946 | GTGCTAGGATTACAGGC | ACT |
| rs4934599 | CACAGAGCTCACCCACC | ACT |
| rs4934934 | AGGAAAAAGGCTTATCAAG | ACT |
| rs2776944 | ACTTGACCTAGCACCAG | ACT |
| rs2776943 | GGCTAAAAAGTGTGTCAA | ACG |
| rs2804886 | GATGTGCTCAGCTCTTA | ACG |
| rs2804900 | CTGTAATCTCCGCCTCC | ACG |
| rs2804902 | GGCTGGAGTGCAGTGGC | ACT |
| rs2768397 | CTGGCTAGCCTTTTAGAA | ACT |
| rs2768399 | ACAAAAACCACCATCAAA | ACT |
| rs2776941 | ATGCCAACTGAGAAGGA | ACT |
| rs2804888 | CTCCCTTCTGGGAGGAT | ACG |
| rs2804499 | CCCAATCCTCCCAGAAG | ACT |
| rs2804889 | GGTCTCCAAATGAGTGG | ACG |
| rs2804891 | GAAACAGGGATGGAGATA | ACG |
| rs2776940 | CAGCAACAGGTCTCACC | CGT |
| rs2804897 | GAGAGGAATGGAGAAAAA | ACG |
| rs2804896 | GTACTGGGATTACAGGC | ACT |
| rs2776939 | AAACGTCAGAGCCAGCC | ACG |
| rs2804894 | TCTCACTGGGGACCCAC | ACT |
| rs2804501 | GCATTTTCCTCTCTCTG | CGT |
| rs2183715 | GATAGAGGTCAAGGAGAG | ACT |
| rs2254822 | GTAAGCCCTACCTATGC | ACG |
| rs2150686 | GTTGCCTGTGTTCTGGA | ACG |
| rs2804893 | GCAATTCTCCTGCCTCA | ACT |
| rs2804502 | GTGAATATGGAAAAAACCAA | ACT |
| rs2804892 | TGCCATCTTCTCATTGT | ACT |
| rs2183714 | TTGTGTAGAATAGAGCCC | ACG |
| rs2804503 | TTGTCCTCTGTGCTAGA | ACG |
| rs1320485 | TGCTGGTGAGCAGGGAG | ACT |
| rs4934603 | GCAGAAGGCGAAGTGGT | ACT |
| rs2804890 | GTGCAGTACCTCCCCCC | ACG |
| rs1854145 | CTCATTTTCTCTCTATTCCC | ACT |

Genetic Analysis

Allelotyping results are shown for female (F) and male (M) cases and controls in Table 14 and Table 15, respectively. Allele frequency is noted in the fourth and fifth columns for melanoma pools and control pools, respectively.

TABLE 14
Females

| | | | | | | | | Melanoma |
|---|---|---|---|---|---|---|---|---|
| | Chromo- | Chromosome | | Female Case | Female Control | F | Odds | Associated |
| dbSNP rs# | some | Position | Alleles | AF | AF | p-Value | Ratio | Allele |
| rs6481844 | 10 | 33588137 | T/C | T = 0.587 | T = 0.612 | 0.440 | 1.110 | C |
| | | | | C = 0.412 | C = 0.387 | | | |
| rs7910405 | 10 | 33590041 | T/C | T = 0.926 | T = 0.890 | 0.090 | 0.640 | T |
| | | | | C = 0.073 | C = 0.109 | | | |
| rs5784331 | 10 | 33590861 | —/A | — = 0.375 | — = 0.386 | 0.725 | 1.049 | A |
| | | | | A = 0.624 | A = 0.613 | | | |
| rs6481845 | 10 | 33596683 | T/C | T = 0.329 | T = 0.305 | 0.445 | 0.896 | T |
| | | | | C = 0.670 | C = 0.694 | | | |
| rs2065364 | 10 | 33598008 | A/G | A = 0.354 | A = 0.396 | 0.192 | 1.197 | G |
| | | | | G = 0.645 | G = 0.603 | | | |
| rs10643659 | 10 | 33599549 | —/GTTT | — = 0.198 | — = 0.125 | 0.006 | 0.582 | — |
| | | | | GTTT = 0.801 | GTTT = 0.874 | | | |
| rs7896887 | 10 | 33603031 | A/G | A = 0.631 | A = 0.571 | 0.066 | 0.778 | A |
| | | | | G = 0.368 | G = 0.428 | | | |
| rs7086456 | 10 | 33606418 | C/T | C = 0.857 | C = 0.853 | 0.870 | 0.965 | C |
| | | | | T = 0.142 | T = 0.146 | | | |
| rs6481846 | 10 | 33606937 | G/A | G = 0.849 | G = 0.837 | 0.663 | 0.919 | G |
| | | | | A = 0.150 | A = 0.162 | | | |
| rs7475391 | 10 | 33606955 | C/T | C = 0.705 | C = 0.608 | 0.014 | 0.647 | C |
| | | | | T = 0.294 | T = 0.391 | | | |
| rs7477281 | 10 | 33606956 | C/T | C = 0.291 | C = 0.236 | 0.079 | 0.752 | C |
| | | | | T = 0.708 | T = 0.763 | | | |
| rs7475393 | 10 | 33606959 | C/T | C = 0.549 | C = 0.486 | 0.159 | 0.777 | C |
| | | | | T = 0.450 | T = 0.513 | | | |
| rs7475394 | 10 | 33606966 | C/T | C = 0.729 | C = 0.815 | 0.007 | 1.641 | T |
| | | | | T = 0.270 | T = 0.184 | | | |
| rs7477282 | 10 | 33606970 | C/T | C = 0.908 | C = 0.899 | 0.738 | 0.903 | C |
| | | | | T = 0.091 | T = 0.100 | | | |
| rs7475396 | 10 | 33606978 | C/T | C = 0.209 | C = 0.104 | ˜0.001 | 0.438 | C |
| | | | | T = 0.790 | T = 0.895 | | | |
| rs2768420 | 10 | 33609638 | T/C | T = 0.789 | T = 0.791 | 0.925 | 1.015 | C |
| | | | | C = 0.210 | C = 0.208 | | | |
| rs9418057 | 10 | 33612486 | C/T | C = 0.865 | C = 0.916 | 0.019 | 1.698 | T |
| | | | | T = 0.134 | T = 0.083 | | | |
| rs7070290 | 10 | 33615210 | G/A | G = 0.948 | G = 0.941 | 0.725 | 0.879 | G |
| | | | | A = 0.051 | A = 0.058 | | | |
| rs1360457 | 10 | 33620935 | T/C | T = 0.278 | T = 0.210 | 0.020 | 0.692 | T |
| | | | | C = 0.721 | C = 0.789 | | | |
| rs1411924 | 10 | 33623568 | G/A | G = 0.165 | G = 0.126 | 0.110 | 0.729 | G |
| | | | | A = 0.834 | A = 0.873 | | | |
| rs5784335 | 10 | 33626177 | T/— | T = 0.219 | T = 0.129 | ˜0.001 | 0.526 | T |
| | | | | — = 0.780 | — = 0.870 | | | |
| rs2776925 | 10 | 33572278 | C/T | C = 0.436 | C = 0.452 | 0.608 | 1.071 | T |
| | | | | T = 0.563 | T = 0.547 | | | |
| rs2776924 | 10 | 33572524 | G/C | G = 0.755 | G = 0.724 | 0.307 | 0.850 | G |
| | | | | C = 0.244 | C = 0.275 | | | |
| rs1952983 | 10 | 33572941 | A/C | A = 0.616 | A = 0.553 | 0.081 | 0.771 | A |
| | | | | C = 0.383 | C = 0.446 | | | |
| rs869636 | 10 | 33576045 | C/T | C = 0.442 | C = 0.444 | 0.949 | 1.009 | T |
| | | | | T = 0.557 | T = 0.555 | | | |
| rs869637 | 10 | 33576211 | C/T | C = 0.888 | C = 0.914 | 0.225 | 1.338 | T |
| | | | | T = 0.111 | T = 0.085 | | | |
| rs2804470 | 10 | 33577636 | G/A | G = 0.181 | G = 0.240 | 0.071 | 1.422 | A |
| | | | | A = 0.818 | A = 0.759 | | | |
| rs2776928 | 10 | 33578411 | T/C | T = 0.893 | T = 0.920 | 0.177 | 1.385 | C |
| | | | | C = 0.106 | C = 0.079 | | | |
| rs999966 | 10 | 33578691 | C/T | C = 0.828 | C = 0.828 | 0.995 | 1.001 | T |
| | | | | T = 0.171 | T = 0.171 | | | |
| rs2804475 | 10 | 33578693 | C/G | C = 0.035 | C = 0.061 | 0.177 | 1.808 | G |
| | | | | G = 0.964 | G = 0.938 | | | |
| rs4934858 | 10 | 33582294 | C/T | C = 0.505 | C = 0.525 | 0.552 | 1.084 | T |
| | | | | T = 0.494 | T = 0.474 | | | |
| rs4934863 | 10 | 33583084 | C/T | C = 0.190 | C = 0.189 | 0.966 | 0.992 | C |
| | | | | T = 0.809 | T = 0.810 | | | |
| rs4934864 | 10 | 33583094 | C/T | C = 0.536 | C = 0.508 | 0.426 | 0.892 | C |
| | | | | T = 0.463 | T = 0.491 | | | |
| rs1331326 | 10 | 33583896 | G/A | G = 0.437 | G = 0.465 | 0.436 | 1.118 | A |
| | | | | A = 0.562 | A = 0.534 | | | |
| rs1331325 | 10 | 33584175 | A/G | A = 0.261 | A = 0.189 | 0.009 | 0.660 | A |
| | | | | G = 0.738 | G = 0.810 | | | |
| rs4934871 | 10 | 33587095 | G/A | G = 0.769 | G = 0.709 | 0.039 | 0.734 | G |
| | | | | A = 0.230 | A = 0.290 | | | |
| rs4277057 | 10 | 33590696 | G/T | G = 0.867 | G = 0.860 | 0.747 | 0.938 | G |
| | | | | T = 0.132 | T = 0.139 | | | |
| rs2776930 | 10 | 33591887 | C/T | C = 0.210 | C = 0.159 | 0.048 | 0.708 | C |
| | | | | T = 0.789 | T = 0.840 | | | |
| rs1331324 | 10 | 33593471 | G/C | G = 0.621 | G = 0.611 | 0.764 | 0.960 | G |
| | | | | C = 0.378 | C = 0.388 | | | |
| rs2776932 | 10 | 33596412 | C/T | C = 0.189 | C = 0.227 | 0.172 | 1.265 | T |
| | | | | T = 0.810 | T = 0.772 | | | |
| rs4934896 | 10 | 33597615 | A/G | A = 0.860 | A = 0.829 | 0.219 | 0.790 | A |
| | | | | G = 0.139 | G = 0.170 | | | |
| rs2776933 | 10 | 33598851 | T/C | T = | T = 0.535 | | | |
| | | | | C = | C = 0.465 | | | |
| rs4934901 | 10 | 33600052 | A/T | A = 0.837 | A = 0.804 | 0.199 | 0.800 | A |
| | | | | T = 0.162 | T = 0.195 | | | |
| rs1015300 | 10 | 33601250 | G/C | G = 0.948 | G = 0.956 | 0.713 | 1.200 | C |
| | | | | C = 0.051 | C = 0.043 | | | |
| rs2776934 | 10 | 33603848 | G/C | G = 0.256 | G = 0.293 | 0.222 | 1.204 | C |
| | | | | C = 0.743 | C = 0.706 | | | |
| rs2776935 | 10 | 33604304 | G/A | G = 0.459 | G = 0.379 | 0.013 | 0.719 | G |
| | | | | A = 0.540 | A = 0.620 | | | |
| rs2776936 | 10 | 33604887 | C/T | C = 0.293 | C = 0.213 | 0.006 | 0.654 | C |
| | | | | T = 0.706 | T = 0.786 | | | |
| rs2804491 | 10 | 33604893 | A/C | A = 0.236 | A = 0.177 | 0.035 | 0.700 | A |
| | | | | C = 0.763 | C = 0.822 | | | |
| rs2804492 | 10 | 33606052 | T/C | T = 0.603 | T = 0.627 | 0.492 | 1.108 | C |
| | | | | C = 0.396 | C = 0.372 | | | |
| rs2804493 | 10 | 33609682 | A/G | A = 0.234 | A = 0.210 | 0.383 | 0.868 | A |
| | | | | G = 0.765 | G = 0.789 | | | |
| rs2776937 | 10 | 33610195 | A/G | A = 0.642 | A = 0.599 | 0.269 | 0.832 | A |
| | | | | G = 0.357 | G = 0.400 | | | |
| rs2804494 | 10 | 33613422 | G/C | G = 0.381 | G = 0.311 | 0.044 | 0.734 | G |
| | | | | C = 0.618 | C = 0.688 | | | |
| rs4934914 | 10 | 33614985 | G/A | G = 0.920 | G = 0.926 | 0.720 | 1.098 | A |
| | | | | A = 0.079 | A = 0.073 | | | |
| rs2776922 | 10 | 33615888 | T/C | T = 0.293 | T = 0.205 | 0.003 | 0.622 | T |
| | | | | C = 0.706 | C = 0.794 | | | |
| rs2804495 | 10 | 33616506 | G/T | G = 0.399 | G = 0.323 | 0.031 | 0.717 | G |
| | | | | T = 0.600 | T = 0.676 | | | |
| rs2804496 | 10 | 33617807 | T/C | T = 0.364 | T = 0.250 | ˜0.001 | 0.582 | T |
| | | | | C = 0.635 | C = 0.749 | | | |
| rs4934597 | 10 | 33618727 | T/C | T = 0.224 | T = 0.197 | 0.334 | 0.849 | T |
| | | | | C = 0.775 | C = 0.802 | | | |
| rs2804497 | 10 | 33620043 | G/T | G = 0.968 | G = 0.970 | 0.891 | 1.068 | T |
| | | | | T = 0.031 | T = 0.029 | | | |
| rs2804498 | 10 | 33624713 | T/C | T = 0.757 | T = 0.770 | 0.644 | 1.077 | C |
| | | | | C = 0.242 | C = 0.229 | | | |
| rs2026319 | 10 | 33625508 | G/C | G = 0.901 | G = 0.908 | 0.742 | 1.083 | C |
| | | | | C = 0.098 | C = 0.091 | | | |
| rs1888684 | 10 | 33626983 | T/C | T = 0.295 | T = 0.228 | 0.051 | 0.707 | T |
| | | | | C = 0.704 | C = 0.771 | | | |
| rs3750733 | 10 | 33627257 | A/G | A = 0.177 | A = | | | |
| | | | | G = 0.823 | G = | | | |
| rs4934927 | 10 | 33632068 | G/C | G = 0.896 | G = 0.906 | 0.608 | 1.127 | C |
| | | | | C = 0.103 | C = 0.093 | | | |
| rs2776946 | 10 | 33636571 | T/C | T = 0.375 | T = 0.329 | 0.190 | 0.818 | T |
| | | | | C = 0.624 | C = 0.670 | | | |
| rs4934599 | 10 | 33637968 | T/C | T = 0.088 | T = 0.074 | 0.479 | 0.834 | T |
| | | | | C = 0.911 | C = 0.925 | | | |
| rs4934934 | 10 | 33638907 | A/G | A = 0.917 | A = 0.915 | 0.911 | 0.973 | A |
| | | | | G = 0.082 | G = 0.084 | | | |
| rs2776944 | 10 | 33639078 | T/G | T = 0.550 | T = 0.520 | 0.448 | 0.887 | T |
| | | | | G = 0.449 | G = 0.479 | | | |
| rs2776943 | 10 | 33640220 | C/T | C = 0.314 | C = 0.259 | 0.077 | 0.765 | C |
| | | | | T = 0.685 | T = 0.740 | | | |
| rs2804886 | 10 | 33640946 | G/A | G = 0.870 | G = 0.909 | 0.077 | 1.495 | A |
| | | | | A = 0.129 | A = 0.090 | | | |
| rs2804900 | 10 | 33641408 | G/A | G = 0.754 | G = 0.739 | 0.644 | 0.925 | G |
| | | | | A = 0.246 | A = 0.261 | | | |
| rs2804902 | 10 | 33641439 | T/C | T = 0.692 | T = 0.680 | 0.694 | 0.945 | T |
| | | | | C = 0.307 | C = 0.319 | | | |
| rs2768397 | 10 | 33642491 | G/C | G = 0.904 | G = 0.934 | 0.187 | 1.515 | C |
| | | | | C = 0.095 | C = 0.065 | | | |
| rs2768399 | 10 | 33642678 | A/G | A = 0.245 | A = 0.202 | 0.172 | 0.782 | A |
| | | | | G = 0.754 | G = 0.797 | | | |
| rs2776941 | 10 | 33643953 | C/G | C = 0.697 | C = 0.731 | 0.265 | 1.185 | G |
| | | | | G = 0.302 | G = 0.268 | | | |
| rs2804888 | 10 | 33645513 | G/A | G = 0.053 | G = 0.072 | 0.290 | 1.392 | A |
| | | | | A = 0.946 | A = 0.927 | | | |
| rs2804499 | 10 | 33645527 | C/G | C = 0.796 | C = 0.835 | 0.184 | 1.298 | G |
| | | | | G = 0.203 | G = 0.164 | | | |
| rs2804889 | 10 | 33645870 | G/A | G = 0.815 | G = 0.859 | 0.083 | 1.380 | A |
| | | | | A = 0.184 | A = 0.140 | | | |
| rs2804891 | 10 | 33646414 | G/A | G = 0.303 | G = 0.232 | 0.015 | 0.696 | G |
| | | | | A = 0.696 | A = 0.767 | | | |
| rs2776940 | 10 | 33647995 | A/T | A = 0.274 | A = 0.197 | 0.009 | 0.654 | A |
| | | | | T = 0.725 | T = 0.802 | | | |
| rs2804897 | 10 | 33648548 | C/T | C = 0.842 | C = 0.900 | 0.011 | 1.691 | T |
| | | | | T = 0.157 | T = 0.099 | | | |
| rs2804896 | 10 | 33649026 | T/C | T = 0.540 | T = 0.495 | 0.193 | 0.835 | T |
| | | | | C = 0.459 | C = 0.504 | | | |
| rs2776939 | 10 | 33649091 | C/T | C = 0.400 | C = 0.359 | 0.214 | 0.841 | C |
| | | | | T = 0.599 | T = 0.640 | | | |
| rs2804894 | 10 | 33651097 | T/C | T = 0.747 | T = 0.797 | 0.078 | 1.328 | C |
| | | | | C = 0.252 | C = 0.202 | | | |
| rs2804501 | 10 | 33652329 | A/T | A = 0.381 | A = 0.373 | 0.817 | 0.969 | A |
| | | | | T = 0.618 | T = 0.626 | | | |
| rs2183715 | 10 | 33652769 | A/C | A = 0.904 | A = 0.907 | 0.884 | 1.037 | C |
| | | | | C = 0.095 | C = 0.092 | | | |
| rs2254822 | 10 | 33652838 | G/A | G = | G = | | | |
| | | | | A = | A = | | | |
| rs2150686 | 10 | 33653029 | G/A | G = 0.116 | G = 0.126 | 0.632 | 1.105 | A |
| | | | | A = 0.883 | A = 0.873 | | | |
| rs2804893 | 10 | 33653545 | T/C | T = 0.173 | T = 0.132 | 0.144 | 0.729 | T |
| | | | | C = 0.827 | C = 0.868 | | | |
| rs2804502 | 10 | 33653866 | T/C | T = 0.765 | T = 0.814 | 0.070 | 1.347 | C |
| | | | | C = 0.234 | C = 0.185 | | | |
| rs2804892 | 10 | 33654085 | T/C | T = 0.871 | T = 0.911 | 0.063 | 1.515 | C |
| | | | | C = 0.128 | C = 0.088 | | | |
| rs2183714 | 10 | 33657076 | G/A | G = 0.267 | G = 0.185 | 0.012 | 0.627 | G |
| | | | | A = 0.732 | A = 0.814 | | | |
| rs2804503 | 10 | 33658722 | C/T | C = 0.416 | C = 0.376 | 0.237 | 0.845 | C |
| | | | | T = 0.583 | T = 0.623 | | | |
| rs1320485 | 10 | 33660041 | T/C | T = 0.403 | T = 0.392 | 0.745 | 0.956 | T |
| | | | | C = 0.596 | C = 0.607 | | | |
| rs4934603 | 10 | 33662649 | A/G | A = 0.585 | A = 0.642 | 0.112 | 1.272 | G |
| | | | | G = 0.414 | G = 0.357 | | | |
| rs2804890 | 10 | 33662689 | C/T | C = | C = 0.559 | | | |
| | | | | T = | T = 0.441 | | | |
| rs1854145 | 10 | 33663027 | A/G | A = 0.042 | A = 0.029 | 0.485 | 0.674 | A |
| | | | | G = 0.957 | G = 0.970 | | | |

TABLE 15
Males

| | | | | | | | | Melanoma |
|---|---|---|---|---|---|---|---|---|
| | Chromo- | Chromosome | | Male Case | Male Control | M | Odds | Associated |
| dbSNP rs# | some | Position | Alleles | AF | AF | p-Value | Ratio | Allele |
| rs6481844 | 10 | 33588137 | T/C | T = 0.625 | T = 0.575 | 0.134 | 0.810 | T |
| | | | | C = 0.374 | C = 0.424 | | | |
| rs7910405 | 10 | 33590041 | T/C | T = 0.906 | T = 0.872 | 0.129 | 0.703 | T |
| | | | | C = 0.093 | C = 0.127 | | | |
| rs5784331 | 10 | 33590861 | —/A | — = 0.386 | — = 0.386 | 0.998 | 1.000 | A |
| | | | | A = 0.613 | A = 0.613 | | | |
| rs6481845 | 10 | 33596683 | T/C | T = 0.342 | T = 0.252 | 0.004 | 0.647 | T |
| | | | | C = 0.657 | C = 0.747 | | | |
| rs2065364 | 10 | 33598008 | A/G | A = 0.397 | A = 0.338 | 0.086 | 0.775 | A |
| | | | | G = 0.602 | G = 0.661 | | | |
| rs10643659 | 10 | 33599549 | —/GTTT | — = 0.185 | — = 0.123 | 0.022 | 0.617 | — |
| | | | | GTTT = 0.814 | GTTT = 0.876 | | | |
| rs7896887 | 10 | 33603031 | A/G | A = 0.600 | A = 0.560 | 0.256 | 0.845 | A |
| | | | | G = 0.399 | G = 0.439 | | | |
| rs7086456 | 10 | 33606418 | C/T | C = 0.838 | C = 0.866 | 0.321 | 1.247 | T |
| | | | | T = 0.161 | T = 0.133 | | | |
| rs6481846 | 10 | 33606937 | G/A | G = 0.810 | G = 0.863 | 0.059 | 1.474 | A |
| | | | | A = 0.189 | A = 0.136 | | | |
| rs7475391 | 10 | 33606955 | C/T | C = 0.675 | C = 0.466 | ˜0.001 | 0.419 | C |
| | | | | T = 0.324 | T = 0.533 | | | |
| rs7477281 | 10 | 33606956 | C/T | C = 0.271 | C = 0.238 | 0.300 | 0.840 | C |
| | | | | T = 0.728 | T = 0.761 | | | |
| rs7475393 | 10 | 33606959 | C/T | C = 0.502 | C = 0.463 | 0.333 | 0.856 | C |
| | | | | T = 0.497 | T = 0.536 | | | |
| rs7475394 | 10 | 33606966 | C/T | C = 0.757 | C = 0.835 | 0.006 | 1.630 | T |
| | | | | T = 0.242 | T = 0.164 | | | |
| rs7477282 | 10 | 33606970 | C/T | C = | C = | | | |
| | | | | T = | T = | | | |
| rs7475396 | 10 | 33606978 | C/T | C = | C = | | | |
| | | | | T = | T = | | | |
| rs2768420 | 10 | 33609638 | T/C | T = 0.780 | T = 0.782 | 0.941 | 1.013 | C |
| | | | | C = 0.219 | C = 0.217 | | | |
| rs9418057 | 10 | 33612486 | C/T | C = 0.870 | C = 0.917 | 0.035 | 1.654 | T |
| | | | | T = 0.129 | T = 0.082 | | | |
| rs7070290 | 10 | 33615210 | G/A | G = 0.941 | G = 0.952 | 0.557 | 1.227 | A |
| | | | | A = 0.058 | A = 0.047 | | | |
| rs1360457 | 10 | 33620935 | T/C | T = 0.264 | T = 0.204 | 0.081 | 0.716 | T |
| | | | | C = 0.735 | C = 0.795 | | | |
| rs1411924 | 10 | 33623568 | G/A | G = 0.134 | G = 0.098 | 0.121 | 0.701 | G |
| | | | | A = 0.865 | A = 0.901 | | | |
| rs5784335 | 10 | 33626177 | T/— | T = 0.183 | T = 0.113 | 0.024 | 0.574 | T |
| | | | | — = 0.816 | — = 0.886 | | | |
| rs2776925 | 10 | 33572278 | C/T | C = 0.440 | C = 0.459 | 0.571 | 1.082 | T |
| | | | | T = 0.559 | T = 0.540 | | | |
| rs2776924 | 10 | 33572524 | G/C | G = 0.751 | G = 0.726 | 0.470 | 0.881 | G |
| | | | | C = 0.248 | C = 0.273 | | | |
| rs1952983 | 10 | 33572941 | A/C | A = 0.613 | A = 0.590 | 0.511 | 0.908 | A |
| | | | | C = 0.386 | C = 0.409 | | | |
| rs869636 | 10 | 33576045 | C/T | C = 0.428 | C = 0.440 | 0.720 | 1.053 | T |
| | | | | T = 0.571 | T = 0.559 | | | |
| rs869637 | 10 | 33576211 | C/T | C = 0.944 | C = 0.941 | 0.908 | 0.958 | C |
| | | | | T = 0.055 | T = 0.058 | | | |
| rs2804470 | 10 | 33577636 | G/A | G = 0.189 | G = 0.167 | 0.415 | 0.858 | G |
| | | | | A = 0.810 | A = 0.832 | | | |
| rs2776928 | 10 | 33578411 | T/C | T = 0.918 | T = 0.936 | 0.361 | 1.295 | C |
| | | | | C = 0.081 | C = 0.063 | | | |
| rs999966 | 10 | 33578691 | C/T | C = 0.822 | C = 0.827 | 0.840 | 1.039 | T |
| | | | | T = 0.177 | T = 0.172 | | | |
| rs2804475 | 10 | 33578693 | C/G | C = 0.052 | C = 0.032 | 0.435 | 0.598 | C |
| | | | | G = 0.947 | G = 0.967 | | | |
| rs4934858 | 10 | 33582294 | C/T | C = | C = | | | |
| | | | | T = | T = | | | |
| rs4934863 | 10 | 33583084 | C/T | C = 0.203 | C = 0.236 | 0.294 | 1.207 | T |
| | | | | T = 0.796 | T = 0.763 | | | |
| rs4934864 | 10 | 33583094 | C/T | C = 0.572 | C = 0.520 | 0.163 | 0.810 | C |
| | | | | T = 0.427 | T = 0.479 | | | |
| rs1331326 | 10 | 33583896 | G/A | G = 0.441 | G = 0.420 | 0.586 | 0.916 | G |
| | | | | A = 0.558 | A = 0.579 | | | |
| rs1331325 | 10 | 33584175 | A/G | A = 0.205 | A = 0.149 | 0.037 | 0.678 | A |
| | | | | G = 0.794 | G = 0.850 | | | |
| rs4934871 | 10 | 33587095 | G/A | G = 0.751 | G = 0.754 | 0.921 | 1.018 | A |
| | | | | A = 0.248 | A = 0.245 | | | |
| rs4277057 | 10 | 33590696 | G/T | G = 0.843 | G = 0.855 | 0.626 | 1.105 | T |
| | | | | T = 0.156 | T = 0.144 | | | |
| rs2776930 | 10 | 33591887 | C/T | C = 0.226 | C = 0.170 | 0.056 | 0.702 | C |
| | | | | T = 0.773 | T = 0.829 | | | |
| rs1331324 | 10 | 33593471 | G/C | G = 0.631 | G = 0.572 | 0.086 | 0.782 | G |
| | | | | C = 0.368 | C = 0.427 | | | |
| rs2776932 | 10 | 33596412 | C/T | C = 0.206 | C = 0.175 | 0.328 | 0.816 | C |
| | | | | T = 0.793 | T = 0.824 | | | |
| rs4934896 | 10 | 33597615 | A/G | A = 0.841 | A = 0.826 | 0.582 | 0.896 | A |
| | | | | G = 0.158 | G = 0.173 | | | |
| rs2776933 | 10 | 33598851 | T/C | T = | T = 0.495 | | | |
| | | | | C = | C = 0.505 | | | |
| rs4934901 | 10 | 33600052 | A/T | A = 0.812 | A = 0.826 | 0.689 | 1.104 | T |
| | | | | T = 0.187 | T = 0.173 | | | |
| rs1015300 | 10 | 33601250 | G/C | G = 0.966 | G = 0.936 | 0.124 | 0.519 | G |
| | | | | C = 0.033 | C = 0.063 | | | |
| rs2776934 | 10 | 33603848 | G/C | G = 0.256 | G = 0.219 | 0.215 | 0.816 | G |
| | | | | C = 0.743 | C = 0.780 | | | |
| rs2776935 | 10 | 33604304 | G/A | G = 0.482 | G = 0.387 | 0.006 | 0.680 | G |
| | | | | A = 0.517 | A = 0.612 | | | |
| rs2776936 | 10 | 33604887 | C/T | C = | C = | | | |
| | | | | T = | T = | | | |
| rs2804491 | 10 | 33604893 | A/C | A = 0.225 | A = 0.167 | 0.047 | 0.694 | A |
| | | | | C = 0.774 | C = 0.832 | | | |
| rs2804492 | 10 | 33606052 | T/C | T = 0.635 | T = 0.587 | 0.186 | 0.817 | T |
| | | | | C = 0.364 | C = 0.412 | | | |
| rs2804493 | 10 | 33609682 | A/G | A = 0.212 | A = 0.210 | 0.935 | 0.986 | A |
| | | | | G = 0.787 | G = 0.789 | | | |
| rs2776937 | 10 | 33610195 | A/G | A = 0.621 | A = 0.582 | 0.303 | 0.849 | A |
| | | | | G = 0.378 | G = 0.417 | | | |
| rs2804494 | 10 | 33613422 | G/C | G = 0.363 | G = 0.314 | 0.138 | 0.802 | G |
| | | | | C = 0.636 | C = 0.685 | | | |
| rs4934914 | 10 | 33614985 | G/A | G = 0.930 | G = 0.949 | 0.279 | 1.405 | A |
| | | | | A = 0.069 | A = 0.050 | | | |
| rs2776922 | 10 | 33615888 | T/C | T = 0.265 | T = 0.223 | 0.164 | 0.796 | T |
| | | | | C = 0.734 | C = 0.776 | | | |
| rs2804495 | 10 | 33616506 | G/T | G = 0.350 | G = 0.322 | 0.392 | 0.883 | G |
| | | | | T = 0.649 | T = 0.677 | | | |
| rs2804496 | 10 | 33617807 | T/C | T = 0.332 | T = 0.212 | 0.001 | 0.542 | T |
| | | | | C = 0.667 | C = 0.787 | | | |
| rs4934597 | 10 | 33618727 | T/C | T = 0.192 | T = 0.160 | 0.235 | 0.801 | T |
| | | | | C = 0.807 | C = 0.839 | | | |
| rs2804497 | 10 | 33620043 | G/T | G = 0.977 | G = 0.995 | 0.108 | 4.348 | T |
| | | | | T = 0.023 | T = 0.005 | | | |
| rs2804498 | 10 | 33624713 | T/C | T = 0.768 | T = 0.777 | 0.759 | 1.053 | C |
| | | | | C = 0.231 | C = 0.222 | | | |
| rs2026319 | 10 | 33625508 | G/C | G = 0.919 | G = 0.949 | 0.310 | 1.636 | C |
| | | | | C = 0.080 | C = 0.050 | | | |
| rs1888684 | 10 | 33626983 | T/C | T = 0.245 | T = 0.161 | 0.038 | 0.590 | T |
| | | | | C = 0.754 | C = 0.838 | | | |
| rs3750733 | 10 | 33627257 | A/G | A = | A = | | | |
| | | | | G = | G = | | | |
| rs4934927 | 10 | 33632068 | G/C | G = 0.908 | G = 0.934 | 0.193 | 1.455 | C |
| | | | | C = 0.091 | C = 0.065 | | | |
| rs2776946 | 10 | 33636571 | T/C | T = 0.351 | T = 0.315 | 0.326 | 0.853 | T |
| | | | | C = 0.648 | C = 0.684 | | | |
| rs4934599 | 10 | 33637968 | T/C | T = 0.084 | T = 0.060 | 0.226 | 0.707 | T |
| | | | | C = 0.915 | C = 0.939 | | | |
| rs4934934 | 10 | 33638907 | A/G | A = 0.923 | A = 0.935 | 0.552 | 1.191 | G |
| | | | | G = 0.076 | G = 0.064 | | | |
| rs2776944 | 10 | 33639078 | T/G | T = | T = | | | |
| | | | | G = | G = | | | |
| rs2776943 | 10 | 33640220 | C/T | C = 0.266 | C = 0.235 | 0.307 | 0.849 | C |
| | | | | T = 0.733 | T = 0.764 | | | |
| rs2804886 | 10 | 33640946 | G/A | G = 0.905 | G = 0.934 | 0.179 | 1.485 | A |
| | | | | A = 0.094 | A = 0.065 | | | |
| rs2804900 | 10 | 33641408 | G/A | G = 0.746 | G = 0.738 | 0.810 | 0.957 | G |
| | | | | A = 0.254 | A = 0.262 | | | |
| rs2804902 | 10 | 33641439 | T/C | T = 0.710 | T = 0.678 | 0.327 | 0.860 | T |
| | | | | C = 0.289 | C = 0.321 | | | |
| rs2768397 | 10 | 33642491 | G/C | G = 0.951 | G = 0.945 | 0.753 | 0.892 | G |
| | | | | C = 0.048 | C = 0.054 | | | |
| rs2768399 | 10 | 33642678 | A/G | A = 0.200 | A = 0.154 | 0.108 | 0.727 | A |
| | | | | G = 0.799 | G = 0.845 | | | |
| rs2776941 | 10 | 33643953 | C/G | C = 0.739 | C = 0.731 | 0.836 | 0.963 | C |
| | | | | G = 0.260 | G = 0.268 | | | |
| rs2804888 | 10 | 33645513 | G/A | G = 0.034 | G = 0.074 | 0.043 | 2.246 | A |
| | | | | A = 0.965 | A = 0.925 | | | |
| rs2804499 | 10 | 33645527 | C/G | C = 0.855 | C = 0.856 | 0.993 | 1.002 | G |
| | | | | G = 0.144 | G = 0.143 | | | |
| rs2804889 | 10 | 33645870 | G/A | G = 0.854 | G = 0.879 | 0.323 | 1.238 | A |
| | | | | A = 0.145 | A = 0.120 | | | |
| rs2804891 | 10 | 33646414 | G/A | G = 0.282 | G = 0.240 | 0.167 | 0.805 | G |
| | | | | A = 0.717 | A = 0.759 | | | |
| rs2776940 | 10 | 33647995 | A/T | A = 0.235 | A = 0.196 | 0.170 | 0.793 | A |
| | | | | T = 0.764 | T = 0.803 | | | |
| rs2804897 | 10 | 33648548 | C/T | C = 0.892 | C = 0.924 | 0.170 | 1.467 | T |
| | | | | T = 0.107 | T = 0.075 | | | |
| rs2804896 | 10 | 33649026 | T/C | T = 0.453 | T = 0.525 | 0.059 | 1.331 | C |
| | | | | C = 0.546 | C = 0.474 | | | |
| rs2776939 | 10 | 33649091 | C/T | C = 0.370 | C = 0.349 | 0.535 | 0.913 | C |
| | | | | T = 0.629 | T = 0.650 | | | |
| rs2804894 | 10 | 33651097 | T/C | T = 0.779 | T = 0.816 | 0.206 | 1.257 | C |
| | | | | C = 0.220 | C = 0.183 | | | |
| rs2804501 | 10 | 33652329 | A/T | A = 0.357 | A = 0.374 | 0.625 | 1.072 | T |
| | | | | T = 0.642 | T = 0.626 | | | |
| rs2183715 | 10 | 33652769 | A/C | A = 0.921 | A = 0.925 | 0.856 | 1.051 | C |
| | | | | C = 0.078 | C = 0.074 | | | |
| rs2254822 | 10 | 33652838 | G/A | G = 0.420 | G = 0.419 | 0.979 | 0.996 | G |
| | | | | A = 0.579 | A = 0.580 | | | |
| rs2150686 | 10 | 33653029 | G/A | G = 0.106 | G = 0.112 | 0.800 | 1.060 | A |
| | | | | A = 0.893 | A = 0.887 | | | |
| rs2804893 | 10 | 33653545 | T/C | T = 0.145 | T = 0.127 | 0.515 | 0.857 | T |
| | | | | C = 0.855 | C = 0.873 | | | |
| rs2804502 | 10 | 33653866 | T/C | T = 0.799 | T = 0.810 | 0.689 | 1.077 | C |
| | | | | C = 0.200 | C = 0.189 | | | |
| rs2804892 | 10 | 33654085 | T/C | T = 0.903 | T = 0.932 | 0.174 | 1.465 | C |
| | | | | C = 0.096 | C = 0.067 | | | |
| rs2183714 | 10 | 33657076 | G/A | G = 0.219 | G = 0.187 | 0.300 | 0.825 | G |
| | | | | A = 0.780 | A = 0.812 | | | |
| rs2804503 | 10 | 33658722 | C/T | C = 0.385 | C = 0.380 | 0.871 | 0.977 | C |
| | | | | T = 0.614 | T = 0.619 | | | |
| rs1320485 | 10 | 33660041 | T/C | T = 0.411 | T = 0.352 | 0.124 | 0.776 | T |
| | | | | C = 0.588 | C = 0.647 | | | |
| rs4934603 | 10 | 33662649 | A/G | A = 0.591 | A = 0.620 | 0.420 | 1.131 | G |
| | | | | G = 0.408 | G = 0.379 | | | |
| rs2804890 | 10 | 33662689 | C/T | C = 0.476 | C = | | | |
| | | | | T = 0.524 | T = | | | |
| rs1854145 | 10 | 33663027 | A/G | A = 0.019 | A = 0.067 | 0.007 | 3.680 | G |
| | | | | G = 0.981 | G = 0.933 | | | |

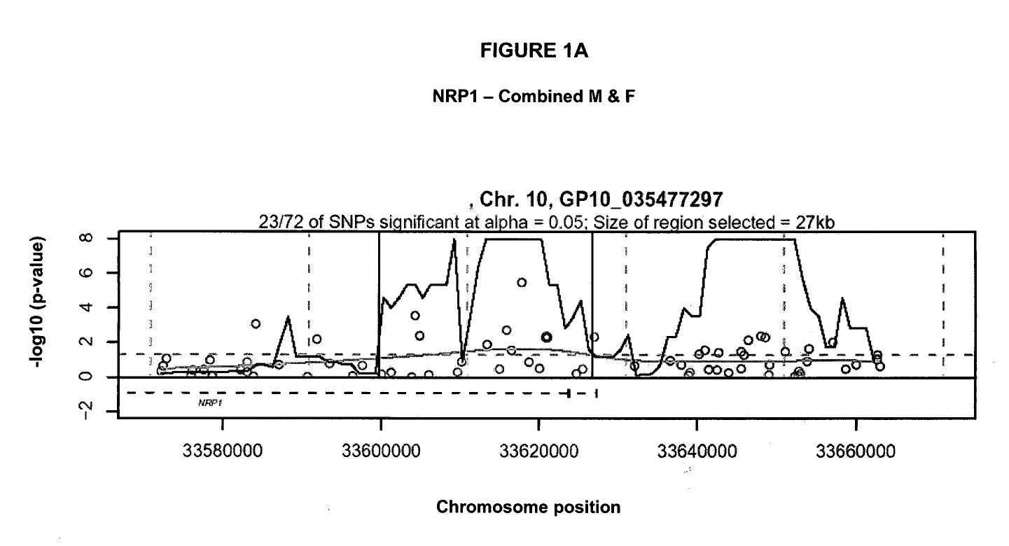

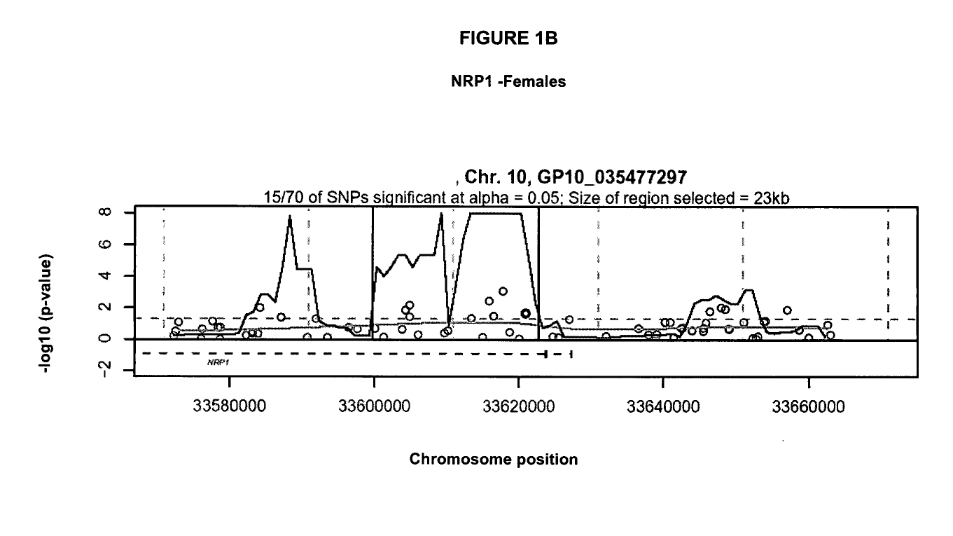

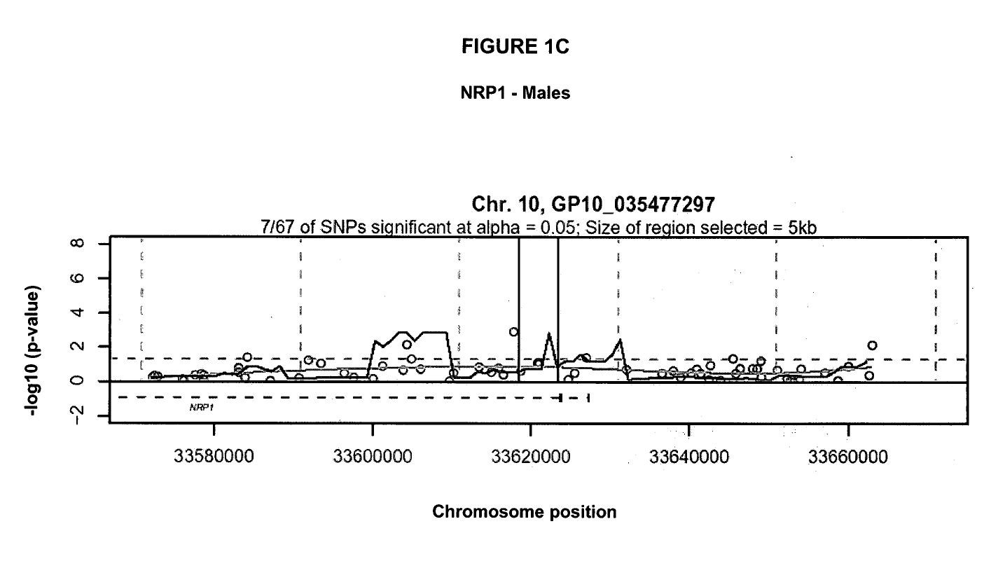

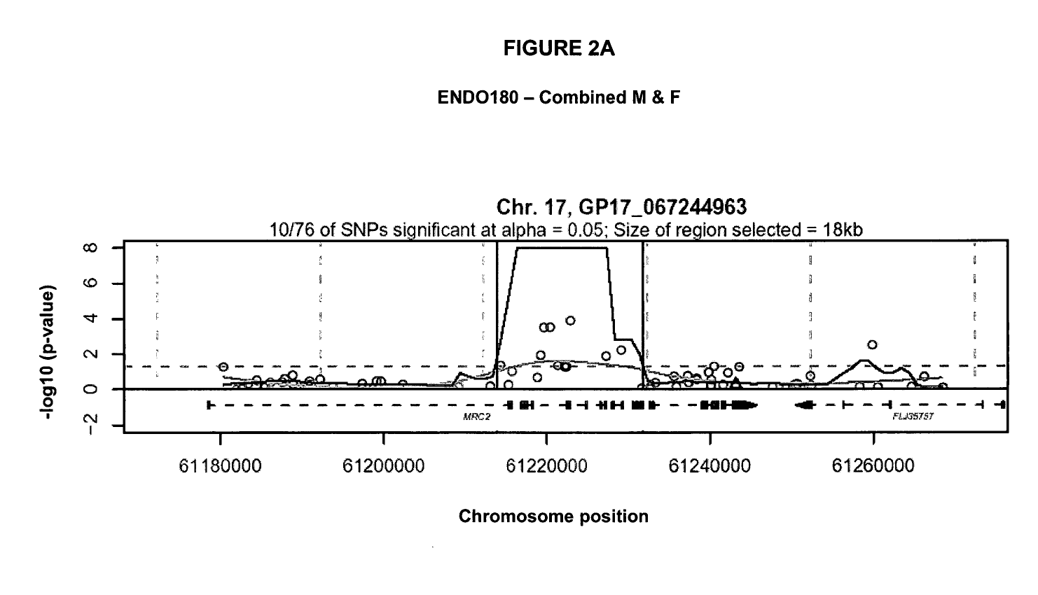

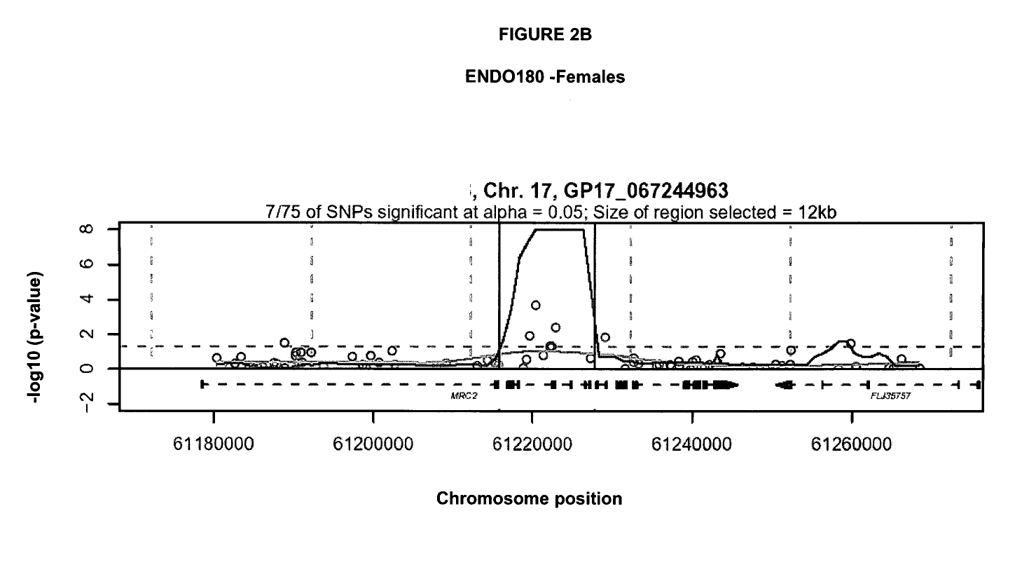

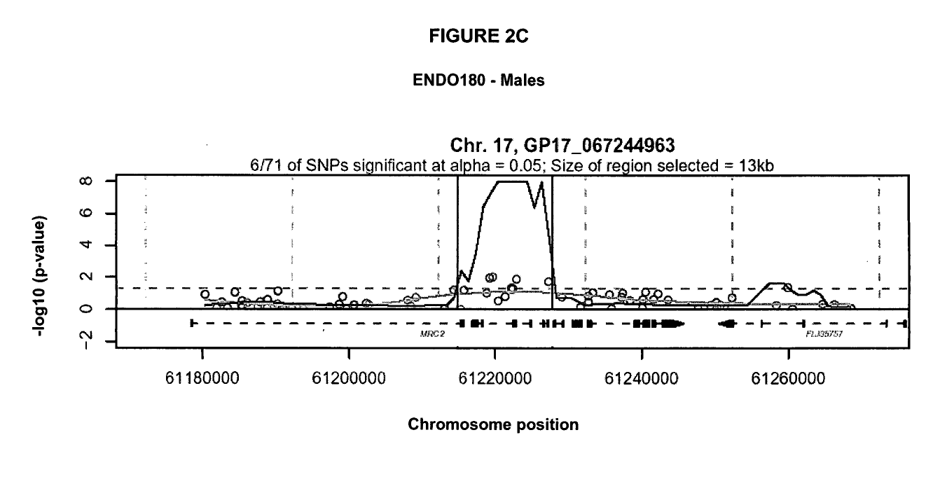

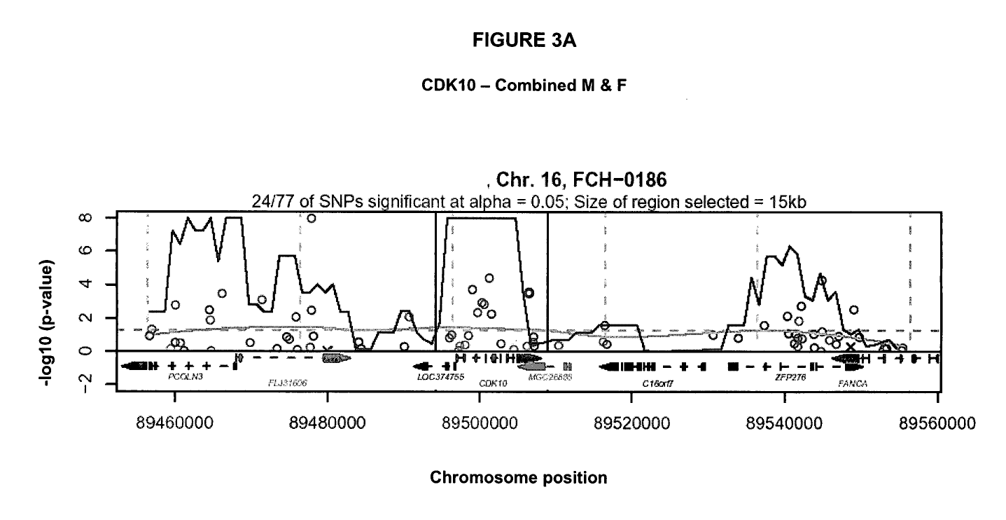

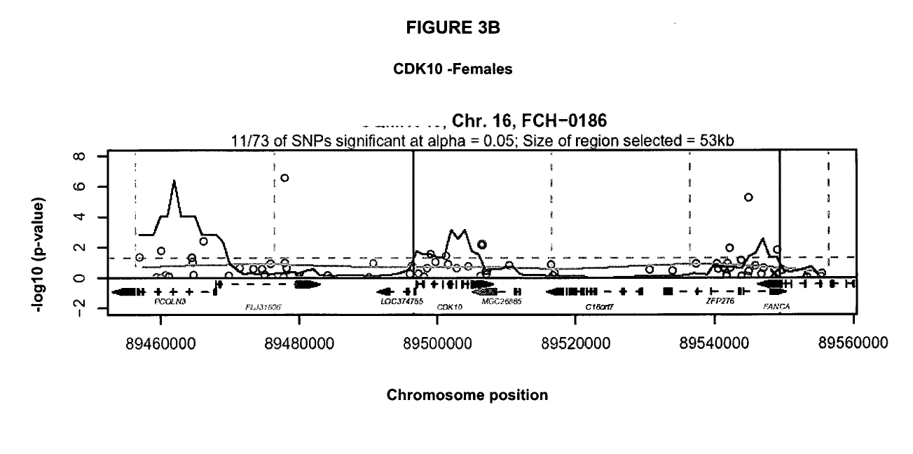

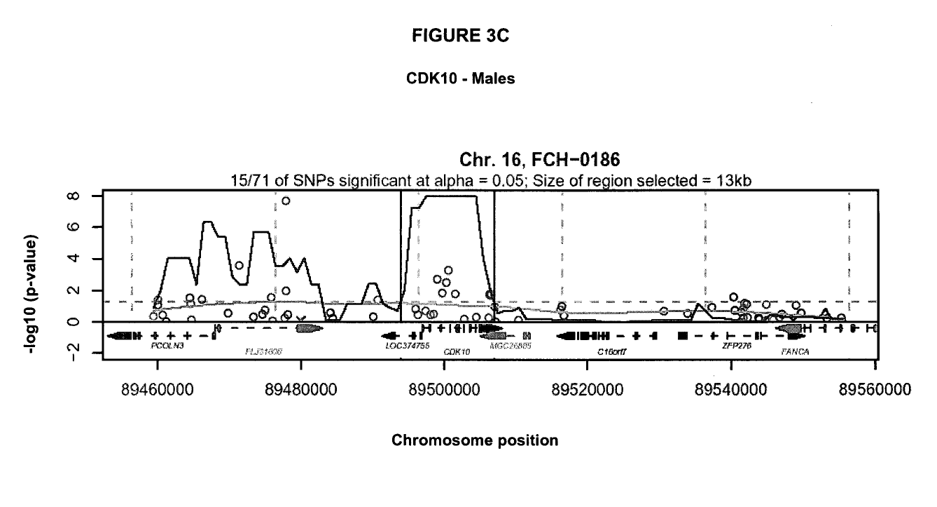

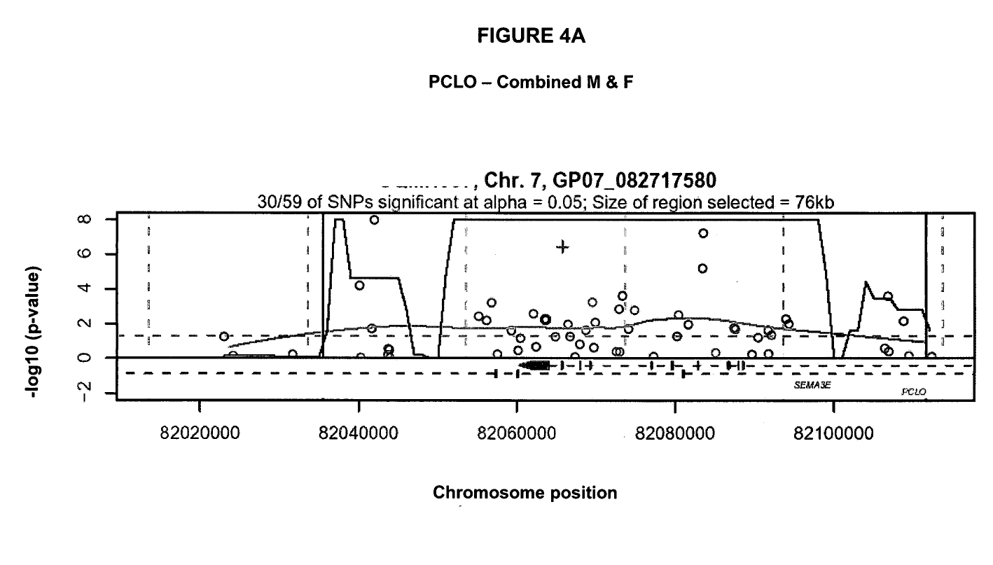

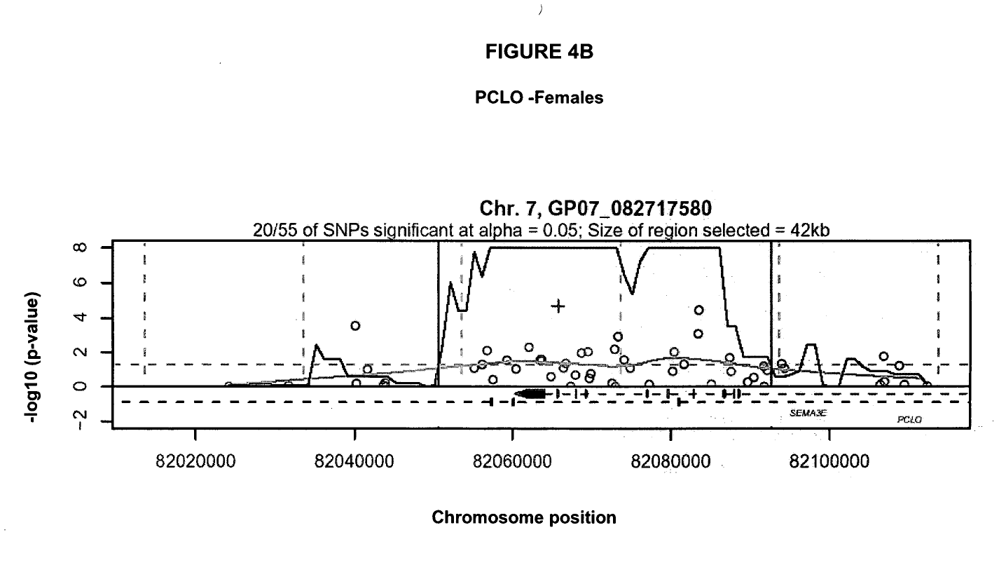

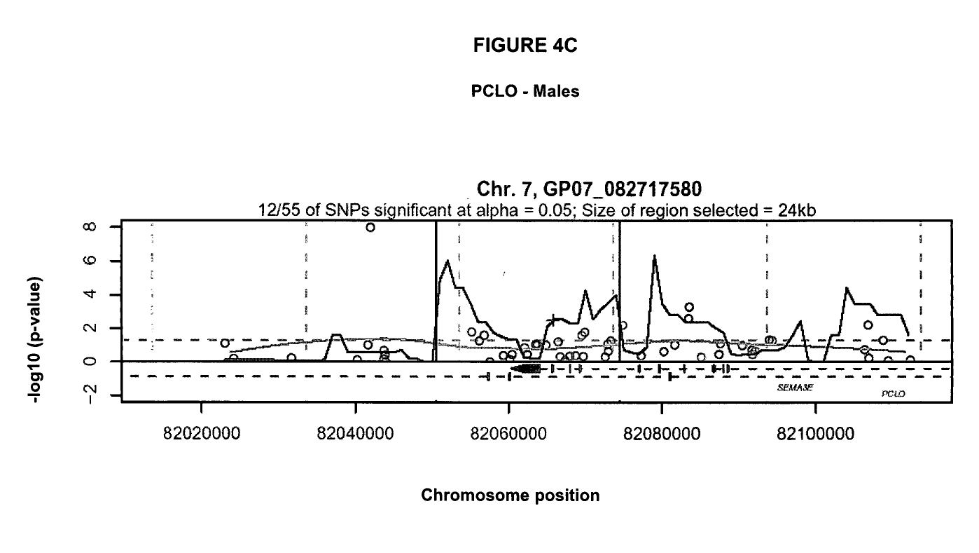

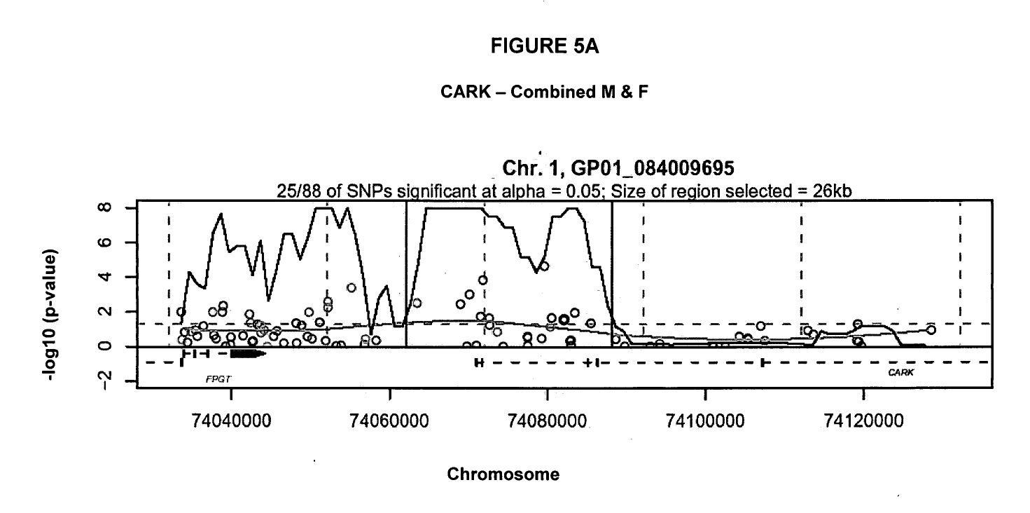

Allelotyping results were considered particularly significant with a calculated p-value of less than or equal to 0.05 for allelotype results. These values are indicated in bold. The assay failed for those SNPs in which the allele frequency is blank. The combined allelotyping p-values for males and females were plotted in FIG. 1A and separately for females and males in FIGS. 1B and 1C, respectively. The position of each SNP on the chromosome is presented on the x-axis. The y-axis gives the negative logarithm (base 10) of the p-value comparing the estimated allele frequency in the case group to that of the control group. The minor allele frequency of the control group for each SNP designated by an X or other symbol on the graphs in FIGS. 1A, 1B and 1C can be determined by consulting Table 14 or 15. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brooks/Cole.). The black line (or generally top-most curve, e.g., see peak in left-most graph just to the left of position 92150000) provides a local test for excess statistical significance to identify regions of association. This was created by use of a 10 kb sliding window with 1 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 1-8 were truncated at that value.

Finally, the exons and introns of the genes in the covered region are plotted below each graph at the appropriate chromosomal positions. The gene boundary is indicated by the broken horizontal line. The exon positions are shown as thick, unbroken bars. An arrow is placed at the 3′ end of each gene to show the direction of transcription.

### Example 6

### ENDO180 Proximal SNPs

It has been discovered that a polymorphic variation in a gene encoding ENDO180 is associated with the occurrence of melanoma.

Forty-eight allelic variants located within or nearby the target gene were identified and subsequently allelotyped in melanoma case and control sample sets as described in Examples 1 and 2. One of the SNPs, designated by SNP_ID “AA” in column 1 of Table 16, is novel and was identified through SNP discovery methods using dye terminator sequencing. Methods of diedeoxynucleotide chain termination chemistry derived sequencing are described by Sanger et al. (Proc. Nat. Acad. Sci. USA 74, 5463-5467, 1997). Briefly, terminators are labeled with fluorescent dyes for automated detection. Fluorescein is measured by an argon ion laser in the sequencing instrument, which transfers light energy to a rhodamine acceptor dye. Each acceptor dye emits light at a characteristic wavelength for detection, identifying the nucleotide that terminated extension of the DNA fragment. Novel SNPs are identified when sequence from multiple samples are compared on one or more strands. The forward and reverse primers used to identify AA are GGCTGACTCAACAGCAGAAG and GGAGGCCTGACAGCAAGG, respectively. All of the polymorphic variants from ENDO180 are set forth in Table 16. The chromosome position provided in column four of Table 16 is based on Genome “Build 34” of NCBI's GenBank and dbSNP 119.

TABLE 16

| | relative_position | | | Snp | Genome | Deduced |
|---|---|---|---|---|---|---|
| SNP Id | in SEQ ID NO: 3 | Chrom | ChromPosition | Alleles | Letter | IUPAC |
| rs2429393 | 224 | 17 | 61200674 | A/C | G | K |
| rs2429392 | 1,874 | 17 | 61202324 | A/G | C | Y |
| rs2465421 | 2,215 | 17 | 61202665 | T/C | T | Y |
| rs2465410 | 7,585 | 17 | 61208035 | T/C | G | R |
| rs2460302 | 8,025 | 17 | 61208475 | C/G | G | S |
| rs2460301 | 8,687 | 17 | 61209137 | A/G | T | Y |
| rs2465459 | 12,603 | 17 | 61213053 | C/G | C | S |
| rs2429391 | 13,885 | 17 | 61214335 | C/T | G | R |
| rs2014055 | 14,838 | 17 | 61215288 | C/T | G | R |
| rs2252814 | 15,269 | 17 | 61215719 | C/A | G | K |
| AA | 16,727 | 17 | 61217177 | G/A | G | R |
| rs8079582 | 18,388 | 17 | 61218838 | C/T | C | Y |
| rs2429390 | 18,792 | 17 | 61219242 | C/T | G | R |
| rs7209331 | 19,195 | 17 | 61219645 | A/G | G | R |
| rs7405815 | 19,925 | 17 | 61220375 | C/T | T | Y |
| rs4968706 | 19,949 | 17 | 61220399 | C/T | T | Y |
| rs4968613 | 20,900 | 17 | 61221350 | C/T | T | Y |
| rs729647 | 21,847 | 17 | 61222297 | G/A | C | Y |
| rs8072984 | 22,454 | 17 | 61222904 | G/A | A | R |
| rs9896444 | 24,193 | 17 | 61224643 | C/T | C | Y |
| rs4968617 | 26,825 | 17 | 61227275 | A/C | A | M |
| rs7350907 | 28,667 | 17 | 61229117 | T/G | T | K |
| rs2429389 | 32,261 | 17 | 61232711 | T/C | A | R |
| rs2460300 | 32,268 | 17 | 61232718 | C/T | A | R |
| rs2465454 | 32,873 | 17 | 61233323 | T/G | A | M |
| rs2465452 | 35,165 | 17 | 61235615 | C/T | G | R |
| rs7219711 | 35,449 | 17 | 61235899 | A/C | C | M |
| rs2465440 | 36,833 | 17 | 61237283 | G/T | G | K |
| rs2429388 | 36,952 | 17 | 61237402 | T/C | A | R |
| rs3786130 | 37,964 | 17 | 61238414 | T/C | T | Y |
| rs2460289 | 39,654 | 17 | 61240104 | G/A | G | R |
| rs2465428 | 39,707 | 17 | 61240157 | C/A | G | K |
| rs740698 | 40,072 | 17 | 61240522 | G/A | C | Y |
| rs2465425 | 41,164 | 17 | 61241614 | T/C | A | R |
| rs2460290 | 41,767 | 17 | 61242217 | T/C | T | Y |
| rs3826537 | 42,724 | 17 | 61243174 | T/C | A | R |
| rs3169756 | 43,139 | 17 | 61243589 | G/T | T | K |
| rs1558237 | 47,241 | 17 | 61247691 | T/C | A | R |
| rs2429385 | 49,720 | 17 | 61250170 | T/C | G | R |
| rs5821368 | 50,036 | 17 | 61250486 | —/T | T | N |
| rs8072538 | 50,836 | 17 | 61251286 | G/A | G | R |
| rs2251393 | 51,853 | 17 | 61252303 | C/T | G | R |
| rs2251391 | 51,946 | 17 | 61252396 | T/C | A | R |
| rs2068657 | 57,864 | 17 | 61258314 | A/G | C | Y |
| rs8072519 | 59,414 | 17 | 61259864 | C/T | C | Y |
| rs7220505 | 60,074 | 17 | 61260524 | C/T | C | Y |
| rs2460288 | 65,721 | 17 | 61266171 | T/C | C | Y |
| rs4968736 | 67,995 | 17 | 61268445 | G/T | T | K |

Assay for Verifying and Allelotyping SNPs

The methods used to verify and allelotype the seventy-six proximal SNPs of Table 16 are the same methods described in Examples 1 and 2 herein. The primers used in these assays are provided in Table 17 and Table 18, respectively.

TABLE 17

| | | |
|---|---|---|
| dbSNP rs # | Forward PCR Primer | Reverse PCR Primer |

| | | |
|---|---|---|
| rs2429393 | GCCTTCTTGTCATTCTTTTT | AGCCGAGATAGTGCCACTC |
| rs2429392 | CTGGGGGACAGAGTGAAACT | CAAGTGTTACTTGAAAGACC |
| rs2465421 | AACAACTTAGATACCCCCCC | CTCAGGAACTAATCATTCTGG |
| rs2465410 | GACCATATGGTGATAAGGGC | GAGAATTTCATAGATGCCAGC |
| rs2460302 | AAGAGAAAAGGCGTCCTGAG | CTCTGCCCGCTTTGTATGTG |
| rs2460301 | TCCTTTGTCACATGAGGCAC | AAAGTTGGGTGGATTTGAAC |
| rs2465459 | TCCTGGGTCCAAATCTCAAG | TGACTGTCTTTGGAGAATGG |
| rs2429391 | TCTTTCCACCTATCTCCTCG | TCTGTAAAGTGGGTGTGCTG |
| rs2014055 | TGCAGTCCATGGCTGAAGAT | TGTGATGCCTTCGTGTCTTG |
| rs2252814 | AATCTGTCCCTCATGGCTCC | ACCTATGTGCTCTGCCCTAC |
| AA | AGAAGGTCTCGCAGTCGTTA | TAGTGTAGCCTTTTGGTGGG |
| rs8079582 | TTAAGGGGTGGTGAGTGATG | CTACTGAGCTCAAGCAATCC |
| rs2429390 | GATTCAATTACTCTGGGGCG | AGTATGGTCATCCAACCAGC |
| rs7209331 | TGGGCAAGCAGCTTAGTCTC | TCACAACAACCTTGCAAGGG |
| rs7405815 | AGTGCAGTGGCACAATCTCC | GAGGCTGGAGCATGAGAATC |
| rs4968706 | TCTCATCTCCAGGTTCAAGC | GTGGCACACTCATGCCTATAA |
| rs4968613 | TTGCCAAGACCAAGTAAGGG | TTCAGGTTCACCTTCTAGGG |
| rs729647 | TCTGGGAGAGTTTGGAGAGG | AACCAACCCACTGGGTTGAC |
| rs8072984 | TAACTAGGATGTCCTTCAGC | ATCTGGGGCCCGGTGAGAT |
| rs9896444 | AACTGCTTGTGCTCTCATCC | TTCTGACATGTCTGTGTCCC |
| rs4968617 | TCCTCGCTTCACCAAGGCAA | GGATGAAGTCATGTACACCC |
| rs7350907 | TGAACAGGATTGTCCTTAGC | CTACTGCCTTTCCCACCATG |
| rs2429389 | TGTACATGACAGCCAGCCGA | ATGACTTCTGCCCAGGCTCG |
| rs2460300 | ACAGACTGATGACTTCTGCC | CAGAGTGCGCAGTCAGCTGT |
| rs2465454 | CACTGATCTAGAACACTCAC | TCACTGTAGTAGCACCTTCC |
| rs2465452 | TGATCTCAGGTGATCCACCC | TTCAAAAGAACATTACACGG |
| rs7219711 | TTTTTAAGGTAGAAGAGGTG | CAGCCTGGGCGAGAGATTGA |
| rs2465440 | AAGAGGCTGTCCCACAAACC | CTCAGGCTGTAATGCCCCTT |
| rs2429388 | CCTGCCATACCCCTTAATAC | TATTCAGCTCAGTCCAGCCG |
| rs3786130 | TAGTAGAGACGGGGTTTCAC | GAAGCTGCTGCTAATAGATC |
| rs2460289 | CCCAGACCAAGGCTTGGTAG | CTCCCTGTCCATTTGTTAGC |
| rs2465428 | AGGCACAAGGCCAGGAGAAG | ACACCTACCAAGCCTTGGTC |
| rs740698 | GTGGGCTCCCGACACTTTTG | AATAGTCAGGATAGGGTGCG |
| rs2465425 | GGGACAGGGAGTCGGTTCTA | AGGAAAGAAGGCACTGAGGG |
| rs2460290 | AGGCATGAGCCACCACATCA | GCCTCAGTTTCCTCGTATGT |
| rs3826537 | CATCCTGGTGTCAGACATGG | AACTGGACAGGCAGCTGGTG |
| rs3169756 | ACAGAGCTGAGGCTGCCAG | CTTCTTTGCAGAGCCCGAGG |
| rs1558237 | ACCACATGGCTCTCCTGGAA | TTTGCCTTGTGCCTGCAATG |
| rs2429385 | CCTGGTTGAAACTACTACTG | CGAGATTGCGCCATCGCACT |
| rs5821368 | CCTACTGGTTGGTTTTCAAG | TCACCTTAAAATCTGGTCCC |
| rs8072538 | TGGCTCATGCCTATAAGCTC | CTATGGCCCAGGCTGATTTC |
| rs2251393 | ACCAAAGCGTTCACAGTTAG | ATAGATGCTCAAGCCAGACC |
| rs2251391 | CTTGGGGACGTAGAAAGAAG | CATTTCTCAAAGCCAGGTCG |
| rs2068657 | ACCTTCAGCTAGGCTCTTCG | GATTGGATGAAAAGCGAGGC |
| rs8072519 | GTCTAGCGTCTAGTAGCTTC | ATTCATGCACTGCCCTGTAC |
| rs7220505 | CCTACTTTCATTTGGGTGCC | TGAGCCATGATTGGTGCCAC |
| rs2460288 | ATGCGGCGAGCCAAATAACA | GTGTAACCTGTGACCTAAGC |
| rs4968736 | AGAAAGCAGCAAGGAACGAG | TGCTGGTGGCTGATCCTTAG |

TABLE 18!Term? ? !dbSNP rs #? Extend Primer? Mix

| | | |
|---|---|---|
| rs2429393 | AGTGTCGCTCTATTGCT | ACT |
| rs2429392 | GAGTGAAACTCGTCTCC | ACT |
| rs2465421 | CAAACAAAAGAAATGTGTAGC | ACT |
| rs2465410 | CAATACCCTCAGTGCCT | ACT |
| rs2460302 | GCTCTGTGTTCTTTGTAC | ACT |
| rs2460301 | ACATGAGGCACAGCTTC | ACT |
| rs2465459 | AGTTCTCTCTCTTCCCC | ACT |
| rs2429391 | ACCCTCTGTAGAAGTCA | ACG |
| rs2014055 | GCTGAAGATGAGGAAGA | ACG |
| rs2252814 | TCCAGTCCCCTGGACAT | CGT |
| AA | CAGTCGTTACCTGCCACCA | ACG |
| rs8079582 | GGCTCACACCTATAATCC | ACG |
| rs2429390 | CATTTCTAACAAGCCCTC | ACG |
| rs7209331 | CTCATTGCAAATGGAGAAT | ACT |
| rs7405815 | GTTCAAGCGATTCTCATC | ACG |
| rs4968706 | TCAAGCGATTCTCATGC | ACG |
| rs4968613 | CCTCAGCTGCCCTCGGA | ACG |
| rs729647 | GAGGTGACTGGGAGGCCT | ACG |
| rs8072984 | ATTTTGTGGCCCTGTGA | ACG |
| rs9896444 | TGAGGCTTATGGGTTCATCC | ACG |
| rs4968617 | CACCAAGGCAAGTCAAA | ACT |
| rs7350907 | TTGTCCTTAGCTAGCCT | ACT |
| rs2429389 | GCGCAGTCAGCTGTGCA | ACT |
| rs2460300 | CCAGGCTCGCCCAGCAT | ACG |
| rs2465454 | CCAAATTATTTGGACAACC | ACT |
| rs2465452 | CCAAAGTGCTGGGATTA | ACG |
| rs7219711 | GAAGAGGTGTAATTTTTTTTTT | ACT |
| rs2465440 | TCTTAGGAGGTGAGCAG | CGT |
| rs2429388 | CCCTTAATACACTTGCAC | ACT |
| rs3786130 | GCGCCCAGCCCTAGGAC | ACT |
| rs2460289 | GGCTTGGTAGGTGTCAG | ACG |
| rs2465428 | CACACCAGGCAAAGACC | CGT |
| rs740698 | CACTTTTGCCCTGGGCC | ACG |
| rs2465425 | AGTCGGTTCTAGAACAC | ACT |
| rs2460290 | CCACCACATCAGTCTGG | ACT |
| rs3826537 | CATGGAAATGAATGAGCA | ACT |
| rs3169756 | CAGGTGGGAAGAGAGACAC | CGT |
| rs1558237 | TCTCCTGGAACGGAGCC | ACT |
| rs2429385 | TTTTTTTGAGATGGAGTTTC | ACT |
| rs5821368 | CATCACTTTAACTGGAACATA | ACG |
| rs8072538 | GGCCAAGGCAGGTAGAC | ACG |
| rs2251393 | CACAGTTAGATTTTCACCT | ACG |
| rs2251391 | TAGAAAGAAGGGCTGGC | ACT |
| rs2068657 | AGGCTCTTCGCGGACAC | ACT |
| rs8072519 | TCCCTGATCCTCATCTC | ACG |
| rs7220505 | GGTCTCACTCTGTCACA | ACG |
| rs2460288 | GGATGAGGTGAAAACAG | ACT |
| rs4968736 | GCAGTGTGGCCAGGGTC | CGT |

Genetic Analysis

Allelotyping results are shown for female (F) and male (M) cases and controls in Table 19 and Table 20, respectively. Allele frequency is noted in the fourth and fifth columns for melanoma pools and control pools, respectively.

TABLE 19
Females

| | | | | | | | | Melanoma |
|---|---|---|---|---|---|---|---|---|
| | Chromo- | Chromosome | | Female Case | Female Control | F | Odds | Associated |
| dbSNP rs# | some | Position | Alleles | AF | AF | p-Value | Ratio | Allele |
| rs2429393 | 17 | 61200674 | A/C | A = 0.754 | A = 0.729 | 0.416 | 0.879 | A |
| | | | | C = 0.245 | C = 0.270 | | | |
| rs2429392 | 17 | 61202324 | A/G | A = 0.753 | A = 0.703 | 0.089 | 0.775 | A |
| | | | | G = 0.246 | G = 0.296 | | | |
| rs2465421 | 17 | 61202665 | T/C | T = 0.456 | T = 0.477 | 0.535 | 1.089 | C |
| | | | | C = 0.543 | C = 0.522 | | | |
| rs2465410 | 17 | 61208035 | T/C | T = 0.873 | T = 0.862 | 0.619 | 0.905 | T |
| | | | | C = 0.126 | C = 0.137 | | | |
| rs2460302 | 17 | 61208475 | C/G | C = 0.500 | C = 0.515 | 0.681 | 1.059 | G |
| | | | | G = 0.499 | G = 0.484 | | | |
| rs2460301 | 17 | 61209137 | A/G | A = 0.482 | A = 0.508 | 0.456 | 1.106 | G |
| | | | | G = 0.517 | G = 0.491 | | | |
| rs2465459 | 17 | 61213053 | C/G | C = 0.688 | C = 0.673 | 0.644 | 0.934 | C |
| | | | | G = 0.311 | G = 0.326 | | | |
| rs2429391 | 17 | 61214335 | C/T | C = 0.473 | C = 0.437 | 0.304 | 0.867 | C |
| | | | | T = 0.526 | T = 0.562 | | | |
| rs2014055 | 17 | 61215288 | C/T | C = 0.807 | C = 0.785 | 0.416 | 0.874 | C |
| | | | | T = 0.192 | T = 0.214 | | | |
| rs2252814 | 17 | 61215719 | C/A | C = 0.562 | C = 0.541 | 0.562 | 0.917 | C |
| | | | | A = 0.437 | A = 0.458 | | | |
| AA | 17 | 61217177 | G/A | G = 0.893 | G = 0.922 | 0.247 | 1.416 | A |
| | | | | A = 0.106 | A = 0.077 | | | |
| rs8079582 | 17 | 61218838 | C/T | C = 0.902 | C = 0.906 | 0.856 | 1.044 | T |
| | | | | T = 0.097 | T = 0.093 | | | |
| rs2429390 | 17 | 61219242 | CAT | C = 0.452 | C = 0.415 | 0.272 | 0.859 | C |
| | | | | T = 0.547 | T = 0.584 | | | |
| rs7209331 | 17 | 61219645 | A/G | A = 0.226 | A = 0.154 | 0.011 | 0.626 | A |
| | | | | G = 0.773 | G = 0.845 | | | |
| rs7405815 | 17 | 61220375 | C/T | C = 0.078 | C = 0.054 | 0.214 | 0.676 | C |
| | | | | T = 0.921 | T = 0.945 | | | |
| rs4968706 | 17 | 61220399 | C/T | C = 0.165 | C = 0.063 | ˜0.001 | 0.340 | C |
| | | | | T = 0.834 | T = 0.936 | | | |
| rs4968613 | 17 | 61221350 | C/T | C = 0.132 | C = 0.100 | 0.157 | 0.731 | C |
| | | | | T = 0.867 | T = 0.899 | | | |
| rs729647 | 17 | 61222297 | A/G | A = 0.700 | A = 0.711 | 0.035 | 1.053 | G |
| | | | | G = 0.299 | G = 0.288 | | | |
| rs8072984 | 17 | 61222904 | G/A | G = 0.216 | G = 0.137 | 0.003 | 0.580 | G |
| | | | | A = 0.783 | A = 0.862 | | | |
| rs9896444 | 17 | 61224643 | C/T | C = 0.893 | C = 0.932 | 0.059 | 1.649 | T |
| | | | | T = 0.106 | T = 0.067 | | | |
| rs4968617 | 17 | 61227275 | A/C | A = 0.695 | A = 0.732 | 0.230 | 1.201 | C |
| | | | | C = 0.304 | C = 0.267 | | | |
| rs7350907 | 17 | 61229117 | T/G | T = 0.774 | T = 0.845 | 0.014 | 1.586 | G |
| | | | | G = 0.225 | G = 0.154 | | | |
| rs2429389 | 17 | 61232711 | T/C | T = 0.011 | T = 0.026 | 0.227 | 2.403 | C |
| | | | | C = 0.989 | C = 0.974 | | | |
| rs2460300 | 17 | 61232718 | C/T | C = 0.983 | C = 0.973 | 0.385 | 0.609 | T |
| | | | | T = 0.167 | T = 0.027 | | | |
| rs2465454 | 17 | 61233323 | T/G | T = 0.186 | T = 0.028 | 0.471 | 1.533 | T |
| | | | | G = 0.981 | G = 0.972 | | | |
| rs2465452 | 17 | 61235615 | C/T | C = 0.047 | C = 0.039 | 0.671 | 0.826 | C |
| | | | | T = 0.952 | T = 0.960 | | | |
| rs7219711 | 17 | 61235899 | A/C | A = 0.809 | A = 0.790 | 0.512 | 0.891 | A |
| | | | | C = 0.190 | C = 0.209 | | | |
| rs2465440 | 17 | 61237283 | G/T | G = 0.672 | G = 0.653 | 0.568 | 0.918 | G |
| | | | | T = 0.327 | T = 0.346 | | | |
| rs2429388 | 17 | 61237402 | T/C | T = 0.030 | T = 0.037 | 0.660 | 1.227 | C |
| | | | | C = 0.969 | C = 0.962 | | | |
| rs3786130 | 17 | 61238414 | T/C | T = 0.733 | T = 0.749 | 0.660 | 1.090 | C |
| | | | | C = 0.266 | C = 0.250 | | | |
| rs2460289 | 17 | 61240104 | G/A | G = 0.045 | G = 0.030 | 0.368 | 0.655 | G |
| | | | | A = 0.954 | A = 0.969 | | | |
| rs2465428 | 17 | 61240157 | C/A | C = 0.016 | C = 0.021 | 0.685 | 1.353 | A |
| | | | | A = 0.984 | A = 0.978 | | | |
| rs740698 | 17 | 61240522 | G/A | G = 0.142 | G = 0.113 | 0.291 | 0.766 | G |
| | | | | A = 0.857 | A = 0.886 | | | |
| rs2465425 | 17 | 61241614 | T/C | T = 0.027 | T = 0.032 | 0.725 | 1.210 | C |
| | | | | C = 0.972 | C = 0.967 | | | |
| rs2460290 | 17 | 61242217 | T/C | T = 0.039 | T = 0.028 | 0.553 | 0.716 | T |
| | | | | C = 0.960 | C = 0.971 | | | |
| rs3826537 | 17 | 61243174 | T/C | T = 0.644 | T = 0.676 | 0.379 | 1.154 | C |
| | | | | C = 0.355 | C = 0.323 | | | |
| rs3169756 | 17 | 61243589 | G/T | G = 0.067 | G = 0.042 | 0.125 | 0.609 | G |
| | | | | T = 0.932 | T = 0.957 | | | |
| rs1558237 | 17 | 61247691 | T/C | T = 0.070 | T = 0.063 | 0.726 | 0.892 | T |
| | | | | C = 0.929 | C = 0.936 | | | |
| rs2429385 | 17 | 61250170 | T/C | T = 0.956 | T = 0.952 | 0.811 | 0.913 | T |
| | | | | C = 0.043 | C = 0.047 | | | |
| rs5821368 | 17 | 61250486 | —/T | — = 0.375 | — = 0.396 | 0.524 | 1.091 | T |
| | | | | T = 0.624 | T = 0.603 | | | |
| rs8072538 | 17 | 61251286 | G/A | G = 0.016 | G = 0.022 | 0.619 | 1.379 | A |
| | | | | A = 0.984 | A = 0.978 | | | |
| rs2251393 | 17 | 61252303 | C/T | C = 0.222 | C = 0.203 | 0.532 | 0.896 | C |
| | | | | T = 0.777 | T = 0.796 | | | |
| rs2251391 | 17 | 61252396 | T/C | T = 0.146 | T = 0.099 | 0.078 | 0.648 | T |
| | | | | C = 0.853 | C = 0.900 | | | |
| rs2068657 | 17 | 61258314 | A/G | A = 0.061 | A = 0.062 | 0.960 | 1.016 | G |
| | | | | G = 0.938 | G = 0.937 | | | |
| rs8072519 | 17 | 61259864 | C/T | C = 0.763 | C = 0.828 | 0.032 | 1.496 | T |
| | | | | T = 0.236 | T = 0.171 | | | |
| rs7220505 | 17 | 61260524 | C/T | C = 0.714 | C = 0.701 | 0.704 | 0.942 | C |
| | | | | T = 0.285 | T = 0.298 | | | |
| rs2460288 | 17 | 61266171 | T/C | T = 0.168 | T = 0.198 | 0.250 | 1.221 | C |
| | | | | C = 0.831 | C = 0.801 | | | |
| rs4968736 | 17 | 61268445 | G/T | G = 0.302 | G = 0.306 | 0.884 | 1.021 | T |
| | | | | T = 0.697 | T = 0.693 | | | |

TABLE 20
Males

| | | | | | | | | Melanoma |
|---|---|---|---|---|---|---|---|---|
| | Chromo- | Chromosome | | Male Case | Male Control | M | Odds | Associated |
| dbSNP rs# | some | Position | Alleles | AF | AF | p-Value | Ratio | Allele |
| rs2429393 | 17 | 61200674 | A/C | A = 0.705 | A = 0.726 | 0.524 | 1.108 | C |
| | | | | C = 0.294 | C = 0.273 | | | |
| rs2429392 | 17 | 61202324 | A/G | A = 0.692 | A = 0.720 | 0.412 | 1.142 | G |
| | | | | G = 0.307 | G = 0.279 | | | |
| rs2465421 | 17 | 61202665 | T/C | T = 0.490 | T = 0.466 | 0.496 | 0.907 | T |
| | | | | C = 0.509 | C = 0.533 | | | |
| rs2465410 | 17 | 61208035 | T/C | T = 0.848 | T = 0.875 | 0.263 | 1.259 | C |
| | | | | C = 0.151 | C = 0.124 | | | |
| rs2460302 | 17 | 61208475 | C/G | C = 0.545 | C = 0.527 | 0.618 | 0.933 | C |
| | | | | G = 0.454 | G = 0.472 | | | |
| rs2460301 | 17 | 61209137 | A/G | A = 0.536 | A = 0.489 | 0.178 | 0.830 | A |
| | | | | G = 0.463 | G = 0.510 | | | |
| rs2465459 | 17 | 61213053 | C/G | C = 0.659 | C = 0.655 | 0.905 | 0.982 | C |
| | | | | G = 0.340 | G = 0.344 | | | |
| rs2429391 | 17 | 61214335 | C/T | C = 0.503 | C = 0.437 | 0.063 | 0.768 | C |
| | | | | T = 0.496 | T = 0.562 | | | |
| rs2014055 | 17 | 61215288 | C/T | C = 0.812 | C = 0.812 | 0.994 | 0.999 | C |
| | | | | T = 0.187 | T = 0.187 | | | |
| rs2252814 | 17 | 61215719 | C/A | C = 0.605 | C = 0.539 | 0.064 | 0.762 | C |
| | | | | A = 0.394 | A = 0.460 | | | |
| AA | 17 | 61217177 | G/A | G = 0.908 | G = 0.943 | 0.067 | 1.685 | A |
| | | | | A = 0.091 | A = 0.056 | | | |
| rs8079582 | 17 | 61218838 | C/T | C = 0.908 | C = 0.940 | 0.095 | 1.594 | T |
| | | | | T = 0.091 | T = 0.059 | | | |
| rs2429390 | 17 | 61219242 | C/T | C = 0.477 | C = 0.390 | 0.011 | 0.701 | C |
| | | | | T = 0.522 | T = 0.609 | | | |
| rs7209331 | 17 | 61219645 | A/G | A = 0.167 | A = 0.100 | 0.009 | 0.558 | A |
| | | | | G = 0.832 | G = 0.899 | | | |
| rs7405815 | 17 | 61220375 | C/T | C = 0.067 | C = 0.045 | 0.212 | 0.658 | C |
| | | | | T = 0.932 | T = 0.954 | | | |
| rs4968706 | 17 | 61220399 | C/T | C = 0.096 | C = 0.071 | 0.293 | 0.725 | C |
| | | | | T = 0.903 | T = 0.928 | | | |
| rs4968613 | 17 | 61221350 | C/T | C = 0.111 | C = 0.081 | 0.155 | 0.706 | C |
| | | | | T = 0.888 | T = 0.918 | | | |
| rs729647 | 17 | 61222297 | A/G | A = 0.660 | A = 0.740 | 0.033 | 1.472 | G |
| | | | | G = 0.339 | G = 0.259 | | | |
| rs8072984 | 17 | 61222904 | G/A | G = 0.161 | G = 0.098 | 0.013 | 0.567 | G |
| | | | | A = 0.838 | A = 0.901 | | | |
| rs9896444 | 17 | 61224643 | C/T | C = 0.917 | C = 0.965 | 0.024 | 2.511 | T |
| | | | | T = 0.082 | T = 0.034 | | | |
| rs4968617 | 17 | 61227275 | A/C | A = 0.681 | A = 0.757 | 0.018 | 1.463 | C |
| | | | | C = 0.318 | C = 0.242 | | | |
| rs7350907 | 17 | 61229117 | T/G | T = 0.804 | T = 0.845 | 0.165 | 1.326 | G |
| | | | | G = 0.195 | G = 0.154 | | | |
| rs2429389 | 17 | 61232711 | T/C | T = 0.055 | T = 0.031 | 0.142 | 0.548 | T |
| | | | | C = 0.944 | C = 0.968 | | | |
| rs2460300 | 17 | 61232718 | CAT | C = 0.961 | C = 0.974 | 0.339 | 1.571 | T |
| | | | | T = 0.038 | T = 0.025 | | | |
| rs2465454 | 17 | 61233323 | T/G | T = 0.056 | T = 0.027 | 0.088 | 0.479 | T |
| | | | | G = 0.943 | G = 0.972 | | | |
| rs2465452 | 17 | 61235615 | C/T | C = 0.057 | C = 0.032 | 0.117 | 0.545 | C |
| | | | | T = 0.942 | T = 0.967 | | | |
| rs7219711 | 17 | 61235899 | A/C | A = 0.789 | A = 0.793 | 0.903 | 1.023 | C |
| | | | | C = 0.210 | C = 0.206 | | | |
| rs2465440 | 17 | 61237283 | G/T | G = 0.672 | G = 0.625 | 0.173 | 0.813 | G |
| | | | | T = 0.327 | T = 0.374 | | | |
| rs2429388 | 17 | 61237402 | T/C | T = 0.053 | T = 0.026 | 0.096 | 0.492 | T |
| | | | | C = 0.946 | C = 0.973 | | | |
| rs3786130 | 17 | 61238414 | T/C | T = 0.754 | T = 0.796 | 0.272 | 1.270 | C |
| | | | | C = 0.245 | C = 0.203 | | | |
| rs2460289 | 17 | 61240104 | G/A | G = 0.039 | G = 0.044 | 0.806 | 1.123 | A |
| | | | | A = 0.960 | A = 0.955 | | | |
| rs2465428 | 17 | 61240157 | C/A | C = 0.047 | C = 0.028 | 0.236 | 0.590 | C |
| | | | | A = 0.952 | A = 0.971 | | | |
| rs740698 | 17 | 61240522 | G/A | G = 0.130 | G = 0.083 | 0.081 | 0.602 | G |
| | | | | A = 0.869 | A = 0.916 | | | |
| rs2465425 | 17 | 61241614 | T/C | T = 0.040 | T = 0.019 | 0.237 | 0.470 | T |
| | | | | C = 0.960 | C = 0.981 | | | |
| rs2460290 | 17 | 61242217 | T/C | T = 0.084 | T = 0.048 | 0.105 | 0.551 | T |
| | | | | C = 0.915 | C = 0.951 | | | |
| rs3826537 | 17 | 61243174 | T/C | T = | T = | | | |
| | | | | C = | C = | | | |
| rs3169756 | 17 | 61243589 | G/T | G = 0.050 | G = 0.031 | 0.250 | 0.619 | G |
| | | | | T = 0.949 | T = 0.968 | | | |
| rs1558237 | 17 | 61247691 | T/C | T = 0.071 | T = 0.068 | 0.891 | 0.960 | T |
| | | | | C = 0.928 | C = 0.931 | | | |
| rs2429385 | 17 | 61250170 | T/C | T = 0.934 | T = 0.950 | 0.366 | 1.342 | C |
| | | | | C = 0.065 | C = 0.049 | | | |
| rs5821368 | 17 | 61250486 | —/T | — = 0.399 | — = 0.407 | 0.808 | 1.034 | T |
| | | | | T = 0.600 | T = 0.592 | | | |
| rs8072538 | 17 | 61251286 | G/A | G = 0.029 | G = 0.023 | 0.646 | 0.786 | G |
| | | | | A = 0.970 | A = 0.976 | | | |
| rs2251393 | 17 | 61252303 | C/T | C = 0.215 | C = 0.178 | 0.187 | 0.793 | C |
| | | | | T = 0.784 | T = 0.821 | | | |
| rs2251391 | 17 | 61252396 | T/C | T = | T = | | | |
| | | | | C = | C = | | | |
| rs2068657 | 17 | 61258314 | A/G | A = 0.072 | A = 0.060 | 0.557 | 0.832 | A |
| | | | | G = 0.927 | G = 0.939 | | | |
| rs8072519 | 17 | 61259864 | C/T | C = 0.793 | C = 0.848 | 0.042 | 1.457 | T |
| | | | | T = 0.206 | T = 0.151 | | | |
| rs7220505 | 17 | 61260524 | C/T | C = 0.710 | C = 0.707 | 0.944 | 0.989 | C |
| | | | | T = 0.289 | T = 0.292 | | | |
| rs2460288 | 17 | 61266171 | T/C | T = 0.169 | T = 0.187 | 0.505 | 1.131 | C |
| | | | | C = 0.830 | C = 0.812 | | | |
| rs4968736 | 17 | 61268445 | G/T | G = 0.277 | G = 0.283 | 0.850 | 1.029 | T |
| | | | | T = 0.722 | T = 0.716 | | | |

Allelotyping results were considered particularly significant with a calculated p-value of less than or equal to 0.05 for allelotype results. These values are indicated in bold. The assay failed for those SNPs in which the allele frequency is blank. The combined allelotyping p-values for males and females were plotted in FIG. 2A and separately for females and males in FIGS. 2B and 2C, respectively. The position of each SNP on the chromosome is presented on the x-axis. The y-axis gives the negative logarithm (base 10) of the p-value comparing the estimated allele frequency in the case group to that of the control group. The minor allele frequency of the control group for each SNP designated by an X or other symbol on the graphs in FIGS. 2A, 2B and 2C can be determined by consulting Table 19 or 20. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brooks/Cole.). The black line (or generally top-most curve, e.g., see peak in left-most graph just to the left of position 92150000) provides a local test for excess statistical significance to identify regions of association. This was created by use of a 10 kb sliding window with 1 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 10⁻⁸ were truncated at that value.

Finally, the exons and introns of the genes in the covered region are plotted below each graph at the appropriate chromosomal positions. The gene boundary is indicated by the broken horizontal line. The exon positions are shown as thick, unbroken bars. An arrow is placed at the 3′ end of each gene to show the direction of transcription.

### Example 7

### CDK10 Proximal SNPs

It has been discovered that a polymorphic variation (rs8404) in the untranslated region of a gene encoding cyclin-dependent kinase 10 (CDK10) is associated with the occurrence of melanoma. See Table 3. Subsequently, one-hundred-three allelic variants located within or nearby the target gene were identified and subsequently allelotyped in melanoma case and control sample sets as described in Examples 1 and 2. Several SNPs, designated by SNP_ID “AA”, “AB”, and “AC” in column 1 of Table 10, are novel and were identified through SNP discovery methods using dye terminator sequencing. Methods of diedeoxynucleotide chain termination chemistry derived sequencing are described by Sanger et al. (Proc. Nat. Acad. Sci. USA 74, 5463-5467, 1997). Briefly, terminators are labeled with fluorescent dyes for automated detection. Fluorescein is measured by an argon ion laser in the sequencing instrument, which transfers light energy to a rhodamine acceptor dye. Each acceptor dye emits light at a characteristic wavelength for detection, identifying the nucleotide that terminated extension of the DNA fragment. Novel SNPs are identified when sequence from multiple samples are compared on one or more strands. The forward and reverse primers used to identify AA are TTCTTCTTGCCCAGATGGAG and GGCTAGCTTTGGGAGATGTTT, respectively. The forward and reverse primers used to identify AB are CACAGAACTGAGTGGCTGGA and AGAGCTGAGCCTGCAGTGA, respectively. The forward and reverse primers used to identify AC are GGAGGCTGTGTCCTGTGGAG and CTATGGGAGGCAGGAGCTGA, respectively. The polymorphic variants are set forth in Table 21. The chromosome position provided in column four of Table 21 is based on Genome “Build 34” of NCBI's GenBank and dbSNP build “119”.

TABLE 21

| | | Position | | | Allele | |
|---|---|---|---|---|---|---|
| dbSNP | Chromo- | in SEQ | Chromosome | Allele | Present in | IUPAC |
| rs# | some | ID NO.: 4 | Position | Variants | SEQ ID NO: 4 | Code |
| rs460879 | 16 | 234 | 89456684 | T/C | C | Y |
| rs460984 | 16 | 519 | 89456969 | T/C | C | Y |
| rs2437957 | 16 | 2,993 | 89459443 | C/T | G | R |
| rs3815949 | 16 | 3,261 | 89459711 | C/G | C | S |
| rs2437956 | 16 | 3,596 | 89460046 | G/A | T | Y |
| rs2437955 | 16 | 3,620 | 89460070 | G/T | C | M |
| rs2434872 | 16 | 4,260 | 89460710 | C/G | C | S |
| rs467357 | 16 | 4,742 | 89461192 | G/A | A | R |
| rs258337 | 16 | 8,055 | 89464505 | A/T | T | W |
| rs258336 | 16 | 8,176 | 89464626 | T/G | T | K |
| rs258335 | 16 | 8,289 | 89464739 | A/G | G | R |
| rs164752 | 16 | 9,735 | 89466185 | G/A | G | R |
| rs154663 | 16 | 13,380 | 89469830 | T/C | T | Y |
| rs164753 | 16 | 14,940 | 89471390 | T/C | C | Y |
| rs258332 | 16 | 15,028 | 89471478 | G/A | G | R |
| rs2010623 | 16 | 16,370 | 89472820 | C/T | A | R |
| rs187283 | 16 | 16,681 | 89473131 | G/A | A | R |
| rs258330 | 16 | 16,919 | 89473369 | T/G | T | K |
| rs258328 | 16 | 17,659 | 89474109 | C/T | C | Y |
| rs459920 | 16 | 18,172 | 89474622 | T/C | T | Y |
| rs166297 | 16 | 18,530 | 89474980 | G/A | A | R |
| rs258318 | 16 | 19,395 | 89475845 | T/C | T | Y |
| rs258317 | 16 | 19,583 | 89476033 | C/T | C | Y |
| rs171805 | 16 | 20,959 | 89477409 | G/A | A | R |
| rs2081984 | 16 | 21,271 | 89477721 | C/T | C | Y |
| rs464586 | 16 | 21,433 | 89477883 | C/T | A | R |
| rs460319 | 16 | 21,438 | 89477888 | G/T | G | K |
| rs447735 | 16 | 21,694 | 89478144 | A/G | T | Y |
| rs2377058 | 16 | 22,176 | 89478626 | T/C | A | R |
| rs604868 | 16 | 23,522 | 89479972 | G/A | T | Y |
| rs2118193 | 16 | 27,248 | 89483698 | C/T | G | R |
| rs467035 | 16 | 27,630 | 89484080 | C/G | C | S |
| rs2115401 | 16 | 27,954 | 89484404 | C/T | T | Y |
| rs417323 | 16 | 33,622 | 89490072 | T/G | T | K |
| rs2377233 | 16 | 34,247 | 89490697 | C/G | G | S |
| AA | 16 | 36,589 | 89493039 | G/C | C | S |
| rs9746114 | 16 | 38,672 | 89495122 | C/G | G | S |
| rs3751700 | 16 | 39,539 | 89495989 | G/A | G | R |
| rs2277905 | 16 | 39,846 | 89496296 | A/G | A | R |
| rs397891 | 16 | 40,376 | 89496826 | C/G | G | S |
| rs3794638 | 16 | 40,781 | 89497231 | G/T | C | M |
| rs3794637 | 16 | 40,915 | 89497365 | G/A | C | Y |
| rs258324 | 16 | 41,600 | 89498050 | C/A | T | K |
| rs258323 | 16 | 42,039 | 89498489 | C/G | G | S |
| rs2075880 | 16 | 42,561 | 89499011 | A/G | A | R |
| rs258322 | 16 | 43,248 | 89499698 | C/T | A | R |
| rs258321 | 16 | 43,818 | 89500268 | C/T | A | R |
| rs164744 | 16 | 44,113 | 89500563 | A/C | T | K |
| rs3833829 | 16 | 44,126 | 89500576 | A/— | C | N |
| AB | 16 | 44,544 | 89500994 | T/C | C | Y |
| rs164743 | 16 | 44,776 | 89501226 | A/G | T | Y |
| rs164742 | 16 | 45,070 | 89501520 | C/T | A | R |
| rs187282 | 16 | 46,318 | 89502768 | T/C | A | R |
| rs465507 | 16 | 47,837 | 89504287 | C/G | C | S |
| rs2162943 | 16 | 47,997 | 89504447 | G/A | C | Y |
| AC | 16 | 48,304 | 89504754 | G/C | C | S |
| rs1946482 | 16 | 49,755 | 89506205 | T/C | T | Y |
| rs8404 | 16 | 49,992 | 89506442 | C/T | T | Y |
| rs1045814 | 16 | 50,260 | 89506710 | G/A | C | Y |
| rs4247353 | 16 | 50,559 | 89507009 | T/C | T | Y |
| rs462769 | 16 | 50,608 | 89507058 | A/G | T | Y |
| rs3803690 | 16 | 50,704 | 89507154 | C/G | G | S |
| rs3751696 | 16 | 50,964 | 89507414 | A/T | A | W |
| rs9972864 | 16 | 52,542 | 89508992 | A/G | G | R |
| rs10567226 | 16 | 53,307 | 89509757 | TTT/— | T | N |
| rs417414 | 16 | 53,493 | 89509943 | A/G | C | Y |
| rs154661 | 16 | 53,916 | 89510366 | G/C | G | S |
| rs4785704 | 16 | 59,715 | 89516165 | G/A | A | R |
| rs4785584 | 16 | 59,729 | 89516179 | C/T | T | Y |
| rs4785585 | 16 | 59,750 | 89516200 | C/T | C | Y |
| rs4785705 | 16 | 59,759 | 89516209 | T/C | C | Y |
| rs4785586 | 16 | 59,761 | 89516211 | G/A | G | R |
| rs4785587 | 16 | 59,964 | 89516414 | A/G | G | R |
| rs2016968 | 16 | 60,251 | 89516701 | C/G | C | S |
| rs3751693 | 16 | 61,741 | 89518191 | C/T | G | R |
| rs3809646 | 16 | 74,200 | 89530650 | A/G | C | Y |
| rs4785590 | 16 | 77,512 | 89533962 | G/C | G | S |
| rs2099105 | 16 | 80,900 | 89537350 | A/G | T | Y |
| rs4785708 | 16 | 83,914 | 89540364 | C/T | C | Y |
| rs4785710 | 16 | 84,101 | 89540551 | T/C | C | Y |
| rs4785591 | 16 | 84,858 | 89541308 | C/T | C | Y |
| rs4785592 | 16 | 84,860 | 89541310 | C/G | G | S |
| rs4785593 | 16 | 84,972 | 89541422 | C/T | C | Y |
| rs4785711 | 16 | 85,291 | 89541741 | C/G | C | S |
| rs4785712 | 16 | 85,299 | 89541749 | G/C | C | S |
| rs4785713 | 16 | 85,443 | 89541893 | G/C | C | S |
| rs4785714 | 16 | 85,749 | 89542199 | C/A | C | M |
| rs4785594 | 16 | 85,861 | 89542311 | C/G | G | S |
| rs3803689 | 16 | 87,403 | 89543853 | A/G | C | Y |
| rs4785715 | 16 | 87,480 | 89543930 | G/A | A | R |
| rs3764258 | 16 | 88,359 | 89544809 | C/G | C | S |
| rs4785716 | 16 | 88,472 | 89544922 | T/C | T | Y |
| rs4785717 | 16 | 88,478 | 89544928 | G/A | A | R |
| rs2077001 | 16 | 89,435 | 89545885 | C/T | G | R |
| rs2377049 | 16 | 90,313 | 89546763 | A/C | T | K |
| rs2003522 | 16 | 90,631 | 89547081 | C/G | C | S |
| rs1230 | 16 | 92,200 | 89548650 | C/T | C | Y |
| rs1800359 | 16 | 92,606 | 89549056 | A/G | A | R |
| rs1061646 | 16 | 93,322 | 89549772 | T/C | G | R |
| rs1800358 | 16 | 96,664 | 89553114 | C/T | T | Y |
| rs2286392 | 16 | 96,894 | 89553344 | C/T | A | R |
| rs2074904 | 16 | 98,891 | 89555341 | T/C | A | R |
| rs2074903 | 16 | 99,008 | 89555458 | A/G | T | Y |

Assay for Verifying and Allelotyping SNPs

The methods used to verify and allelotype the one-hundred-three proximal SNPs of Table 21 are the same methods described in Examples 1 and 2 herein. The primers and probes used in these assays are provided in Table 22 and Table 23, respectively.

TABLE 22

| | | |
|---|---|---|
| dbSNP rs # | Forward PCR primer | Reverse PCR primer |

| | | |
|---|---|---|
| rs460879 | TCCAGCCTCTAACCCACAAC | CGAAACGCTGTGTCACCAAC |
| rs460984 | ATGCTCTGGACAACAGGTGG | TGAGGGAACGAAAAGTGCAG |
| rs2437957 | ATCCAGCTTCCCTCAACCTC | CAGCTCAGAGTTGACGGAAG |
| rs3815949 | TTTTTCTGCAGAAGGGCCTG | CACACACCACCAAGCTCTTC |
| rs2437956 | TCAACACTGGGCTCCTAGGG | TCTCCTGACCTCGTGATCTG |
| rs2437955 | TCAACACTGGGCTCCTAGGG | TCTCCTGACCTCGTGATCTG |
| rs2434872 | TAGACACCAGCTCTGTAGGG | ATTACAGGCATGAGCCACTG |
| rs467357 | TCATCTCTGTGCTCTGGACG | TGTGTGGTGCAGCCCAGAAT |
| rs258337 | TAGAATCCAAAAGATGGTGG | TGCTGGGATTACAGGTGAAC |
| rs258336 | CTTTGAGGAGAAAGAGTCTG | TTAAAGCCAGGGTTTGAGGC |
| rs258335 | TCCTGACCTCAAGTGATCTG | AAAGAGCTGATTTCCAGCGG |
| rs164752 | AAGTGCTGGAATTACAGCCG | AAAATATGCTGGCCAGGCCG |
| rs154663 | ATTGCTCGAACCTTGAACCG | GACAGAGTCTCACTCTGTAG |
| rs164753 | AGTGAGAAGAACAGAAGGGC | GCCAAGCCACATCAATATCC |
| rs258332 | TGTGTCCTCACCCAAATCTC | CCTGCCCCCATAATTCAATC |
| rs2010623 | CCATGACGCCCAGCTAATTT | AACCAGGAGTTTGAGGCCAG |
| rs187283 | CCGGGTTCAAGCAATTCTTC | AATTACCCAGGCATGGTGGC |
| rs258330 | GCTTTTTTCACTCAGCATGG | GGTAGAGTATACTTAATTGC |
| rs258328 | TCAGGTTTCACCATGTTGCC | CAGAACTTTGGGAAGCCTAG |
| rs459920 | AACAGCCATGTGTAACCCTC | TAATCGCAGGAGAGACACAC |
| rs166297 | ACTTCATTCCTTGAACCGGG | ACTGCATTCTCATGAGGAGC |
| rs258318 | TGCGTCTGGCCTAATATTTC | CACTATTGAAATTTTCTAGTC |
| rs258317 | TGAACTCCTGACCTCAAGTG | GCCAAGTGTAGTGATTCACG |
| rs171805 | ACCATGGTCTGCTTGGCAAC | GCCTTTTGTTATGCCCAGAC |
| rs2081984 | TTCCAAGTAGCTGGGACCAC | TATTACACTGGAACAGCTCG |
| rs464586 | GACCTTCAAGATCCTGTCTC | ATAGCCTCATCTGTGTCAGC |
| rs460319 | GACCTTCAAGATCCTGTCTC | ATAGCCTCATCTGTGTCAGC |
| rs447735 | ATGGCTTGGCTTGTCTGTAC | CACAGCAGCTTCTGCTATAG |
| rs2377058 | GGGTTAGAAGACTCAGTCAC | TACCAAGTGTCCCACAAAGC |
| rs604868 | CAACCCTGGTGTACCCTCC | CTGCCCCCGCCGTGAGAAA |
| rs2118193 | TTTTTGAGACGGAGTCTCGC | AGGAGAATGGCTGAACCTGG |
| rs467035 | CGTGTCTGTTTCCACTCTTG | CCCTGTGTAGAAAAGAAGGC |
| rs211540 | TTGGCATCTTTGGGCGTTTTG | CAACCCGCTTCAGCCTTGAG |
| rs417323 | GAGGACTGAAGAAAGAATTTG | GCGAGACTCTGTCTCAAATA |
| rs2377233 | TCTCTTTTTGGACTTCAGGG | TCCCACAGTGCTAGGATTAC |
| AA | GGTCCTGCAGATAACAATGC | AGCCGTAGGTATCAGACATG |
| rs9746114 | TGAATGGAGTCTAGGTCTGG | TGTTTATGGCCAGTTTTGCG |
| rs3751700 | TCAGGTCTTCCATGAGTGAG | TTCAAGCTTAGCTTCTGGGC |
| rs2277905 | TCCTCCAGTTGGGAGTCCTT | CACCTGGAAGACTCTCTCAC |
| rs397891 | CGGGAGCGTCTCTTGGTAAC | CAGGCGCGAAAGCTCCTTC |
| rs3794638 | AGCCTGCTCTTCCCAAGTCC | CGGTCCCTGGAGATCTGAG |
| rs3794637 | GAGCCTTCAAGCTCGAACTC | AGCCGCGAGCCACTTGTTTG |
| rs258324 | AAGCAGAGGATGTGAGAAGG | AAGCATCTGGCCTGTCACTC |
| rs258323 | CACGCCCAGCTAATTTTTTC | AGTCAGGAGATCTAGAGCAC |
| rs2075880 | AAGTGCTGGGATTACAAGCG | AAAGCATTGGCCCAGTATCG |
| rs258322 | TCTGAAACAGGGCAGAAACC | GGATGCTTACGTTTACCCAG |
| rs25832 | TTTGGCAACAGAGTGAGACCC | AGCTTCCAGAATGACCTACG |
| rs164744 | TCAGTCCCCACTCTGTGCAA | ACAGAGCAAAGTTGGGCACC |
| rs3833829 | ATGCCCAGTGACACTCAGTC | CCTGCCCAGTTCAAATGAGG |
| AB | ATGGAGACTGCAGAGCCCTC | TGGTGTGGCCCAGCACATCA |
| rs164743 | AAAAGTGCTTATTGGGCCGG | AACTGCCGACCTCAGGTGAT |
| rs164742 | AACGAGCGAAACTCCGTCTC | TGATAGCAGGTGCTGCAGTG |
| rs187282 | TGGGAACCCCTGAGCTTTTA | GTTGGAAGCACAAATTCGGC |
| rs465507 | AAGCACCCACCTCAATACGG | TCCGAGTTGGGACAGGTTTC |
| rs2162943 | TGAGCTCAGGAACCAGGTGAC | CAGCATCGACATGTGGTGAG |
| AC | TGACTGCAGCCAGACCTTTG | TGCCTGTCCTTCACAGTGTC |
| rs1946482 | TCCTCGCTATGTTGGAAATG | ACCACAACTCAAAGACAGCG |
| rs8404 | AAGGTATGGGGTGGGAGC | GCCTCCTGTTGGGTCCTC |
| rs1045814 | AGAGGGGCTGTCACAGTGGA | TGGGTCCAGTGGTAATGGTG |
| rs4247353 | AGCTGCTGTGACACCCAAGG | AAGCTCTGCTGGAGCCAATC |
| rs462769 | AAGCCAGGTCAGCCGTGCA | TCCTCAAGGGCCTGTTGGTG |
| rs3803690 | ACACTCGTGTGGAGAATCCC | TCCCTGCAGTACGGAGTACG |
| rs3751696 | ATACAGAGAGTCCCACCTCC | TCTGGTTGCCTTGTCCCTGG |
| rs9972864 | TACAAGCGTGAGCCACTGTG | ACTGTACTCCAGACAGAGCG |
| rs10567226 | TCCATCCTGGTGACAGAGTG | ACAGCCGTGGGACGGTAGAA |
| rs417414 | CCACACCCAGCTCATTTTTG | GAGATTGACACCAGCCTAAC |
| rs154661 | CCCTTGAGGCCTCCCAGGG | GGCCAGGAAGCAGCAGAAC |
| rs4785704 | CACGCCCGGCTGATTTTTTG | CACGAGGTCAGGAGATCAAG |
| rs4785584 | TAATCCCAGCACTTTGGGAG | GGTTTCACCGTGTTAGCCAG |
| rs4785585 | CCTGTAATCCCAGCACTTTG | AGGATGGTCTTGATCTCCTG |
| rs4785705 | AGGATGGTCTTGATCTCCTG | CCTGTAATCCCAGCACTTTG |
| rs4785586 | ATCTCCTGACCTCGTGATCC | AAATCCTACAAATGGCCGGG |
| rs4785587 | AACAAATCCTGGCGTGGGAG | GGATACAGCTGAGCCAGGAC |
| rs2016968 | GACACTTCTGTCTCCCTATG | AAAAGCAGTAGCTGTGGACC |
| rs3751693 | ATCAGGTACCTGCATGGATG | GCAGACACTACCTTCTGGAG |
| rs3809646 | AAAAATGCACGAACGCACGG | TAGAGGAGATGTAGCAGGAG |
| rs4785590 | ACCTCTTGCTGGTGCTAGAC | CTAGAGCAGTGGAGACATTC |
| rs2099105 | ATCAGAGGCTGCATAGCACC | GATGACTAGAAGGGAGACTG |
| rs4785708 | TGTTCATGCTGAAGCTGCTG | GAAAAGAGAAACGGGAGCAG |
| rs4785710 | TTGTTCCCAGGAGTCATGGC | AGGTGTCCTGGGATTCAGAC |
| rs4785591 | ATTGGGTGGCCCCTTGTTTG | GAGGAGGTGAGTGGTCATTG |
| rs4785592 | ATTGGGTGGCCCCTTGTTTG | GAGGAGGTGAGTGGTCATTG |
| rs4785593 | CGCACTGAGAAGAACTGTTG | GGCTAGGTCATGAAAACCAG |
| rs4785711 | TAAAGAGTGCATTGAGGCCG | AACTCCTGACCTCATGATCC |
| rs4785712 | CAAAGTGCTGGGATTACAGG | AACAACATGAGACCCTGTCG |
| rs4785713 | AAGTGAGCCTCTCACCTCAG | TAGTTTACACTGCCGAGTCC |
| rs4785714 | CTGTGAAGGACTGAAAGCTC | TGAGAGCAGTGGTGAGAGAA |
| rs4785594 | GCACTTGTGGTTCCTTTCTG | TATCGTGGTGAAGAAGTTCC |
| rs3803689 | GTGTTTCTGCAGCCTAACTC | TAGGACTGCTTCAGGTCTCG |
| rs4785715 | TGAAGCAGTCCTAGCCAGTG | AAGAAACTTCTAGACCCCCC |
| rs3764258 | TTCATGCCTTCTGCCTTGCC | ATCTCAGCCTTCCAAAGTGC |
| rs4785716 | ATGAAGGCACACCAGCTCTG | TAATGACACGACCTACACAC |
| rs4785717 | ATGAAGGCACACCAGCTCTG | CGGTCTTAATGACACGACCT |
| rs2077001 | AGGCTAAGGCAGTAGGATTG | TTTGGTAAAGACGGCCTCAC |
| rs2377049 | CACAAAGTGCTCAATCCCAG | ACCATATTGATTGGGCTGGG |
| rs2003522 | CTGAGATTGGGCTGTTGCAC | CTTGGCATTTTACTCTACGC |
| rs1230 | TCTCCTCGACTGCTTTAGTG | CCAGTGGTTTATTTTCCCGC |
| rs1800359 | AAAGAGCTTCTCACACGTGG | GGCAGCTGTCAATTCTCATG |
| rs1061646 | ATAGGCAGAGATGTCCAGAG | AAAAAGCGAAAGGCAGCAGC |
| rs1800358 | TTCTCTCTGTCCCAGTTTCC | AATCGCAAAGTGCAGTGCAG |
| rs2286392 | CAGGTCCACGTGAGAGTGTG | CAGTCAGCAGCTCTCAGAAC |
| rs2074904 | TGTAGTGGCCTGTAGGAGCA | AGATGAGGGTGGCTGAGATG |
| rs2074903 | CGTCAATTAAGGCTCAAAGC | CCTTCTCCAAATTCCACGTC |

TABLE 23

Term

| | | |
|---|---|---|
| dbSNP rs # | Extend Primer | Mix |

| | | |
|---|---|---|
| rs460879 | ACCAACCCCACGCTCTG | ACT |
| rs460984 | CTTGTGAAGCTGAGTGG | ACT |
| rs2437957 | CGGAAGGTGGATCAGCG | ACG |
| rs3815949 | TCCAGCTGCTTTCCCCC | ACT |
| rs2437956 | TCGGCCTCCCAAAGAGC | ACG |
| rs2437955 | ACAGGCGTGAGCCACCG | CGT |
| rs2434872 | GCGGATCCTATCATTTTTA | ACT |
| rs467357 | GTGCAGCCCAGAATGGTTTC | ACG |
| rs258337 | AAAGAATCCCAAGGGAC | CGT |
| rs258336 | CTGGGCCAATTTAAAAGT | ACT |
| rs258335 | TTTCCAGCGGTACAGTC | ACT |
| rs164752 | GCTCAGGCCTGTAACCC | ACG |
| rs154663 | GTGCGATCTCGCTCACC | ACT |
| rs164753 | TCCTAACAGCTGCCACA | ACT |
| rs258332 | CCATAATTCAATCACCTCC | ACG |
| rs2010623 | GCCAACGTGGCGAAACC | ACG |
| rs187283 | GGCGCACACCTGTAGTC | ACG |
| rs258330 | AAGAGAAATGGAACACTTC | ACT |
| rs258328 | CAGGCAGATCACTTGAG | ACG |
| rs459920 | CCTCCCTCTTCCTCATTCC | ACT |
| rs166297 | CTCTTCTAAGCCCATTC | ACG |
| rs258318 | GACTTATTTCATCTTCCTCA | ACT |
| rs258317 | ATTCACGCCTGTAATCC | ACG |
| rs171805 | AGGGCTCCTTGGTCTAG | ACG |
| rs2081984 | GGCACCTGGGTGACTTG | ACG |
| rs464586 | CTAAGCACTGAGCGATA | ACG |
| rs460319 | CTGCACTAAGCACTGAG | CGT |
| rs447735 | TTCTGCTATAGGTCTCTGACA | ACT |
| rs2377058 | CCACAAAGCTGTAAAACA | ACT |
| rs604868 | CGCCGTGAGAAACTGCAG | ACG |
| rs2118193 | GCAGTGAGCTGGGATCG | ACG |
| rs467035 | AAGAAGGCACCAGACTC | ACT |
| rs2115401 | CCTTGAGCACTGGAGTC | ACG |
| rs417323 | ACACTTTTTATATTTGACAAACTT | ACT |
| rs2377233 | GCTAGGATTACAGGTGT | ACT |
| AA | TTGGCCTTTTGCAGTACCTC | ACT |
| rs9746114 | GGCTGTGGGCTGTTGGGAA | ACT |
| rs3751700 | CAGCTATGTCAGCATTC | ACG |
| rs2277905 | CTCTCACCTGGAGGACC | ACT |
| rs397891 | AAGCTCCTTCCCGGGCTTC | ACT |
| rs3794638 | GGCTCAGGGATGCCTCG | CGT |
| rs3794637 | TGTGGGGAGAATTTACA | ACG |
| rs258324 | TGATGGCCTAGTCTCAA | CGT |
| rs258323 | TAGAGCACCCTGACTAA | ACT |
| rs2075880 | CCACCTTTTCTCATCAGA | ACT |
| rs258322 | ACCCAGAAATGGTACAA | ACG |
| rs258321 | GTGGTGTTTTTGGTTTTTT | ACG |
| rs164744 | CCCCTGCCCAGTTCAAA | ACT |
| rs3833829 | CCGAGACGCGTTCCCCC | ACT |
| AB | CCCTCCTGGCAGACAGCT | ACT |
| rs164743 | GTGCTGGGATTACAGGC | ACT |
| rs164742 | CCCCACCTCAGGGAGAA | ACG |
| rs187282 | CACAAATTCGGCTGAGGCCT | ACT |
| rs465507 | GGACAGGTTTCCAGGCCA | ACT |
| rs2162943 | CATGTGGTGAGGAGATA | ACG |
| AC | CCTGCCAGTGTGAGGCAC | ACT |
| rs1946482 | CACCCACCACTCCTCCC | ACT |
| rs8404 | CGAGACTACCAGGAGAGCCC | ACG |
| rs1045814 | CTGTCACAGTGGATGCACC | ACG |
| rs4247353 | CTGACCTGGCTTCGGGG | ACT |
| rs462769 | ACCCCAAGCTCTGCTGG | ACT |
| rs3803690 | GAGTACGGGGCTCAGGA | ACT |
| rs3751696 | GGTTGGCCTGGACTCTCCA | CGT |
| rs9972864 | GCCACTGTGCCCGGCCTA | ACT |
| rs10567226 | GACTCCGTCTCAAAAAAAAAAAAA | ACT |
| rs417414 | CAACATGGTGAAACCCC | ACT |
| rs154661 | GTCCAAACCAGGGCTGTCC | ACT |
| rs4785704 | ATCAAGACCATCCTGGC | ACG |
| rs4785584 | GTTAGCCAGGATGGTCT | ACG |
| rs4785585 | CTCCTGACCTCGTGATC | ACG |
| rs4785705 | CAGCACTTTGGGAGGCC | ACT |
| rs4785586 | CCCAGCACTTTGGGAGG | ACG |
| rs4785587 | TGAGCCAGGACGTTCCC | ACT |
| rs2016968 | CTCTGTGCTTGCACCCT | ACT |
| rs3751693 | GTGTCCTTCTGTTCGTT | ACG |
| rs3809646 | CCAAAATCCGAGTGACG | ACT |
| rs4785590 | TGCTGAAAACCCTGCCC | ACT |
| rs2099105 | CTCAAGTTCTCCCCACC | ACT |
| rs4785708 | GAGCAGTGGAGGAGGCC | ACG |
| rs4785710 | GGATTCAGACTCAACCC | ACT |
| rs4785591 | AGTGGTCATTGTGGGAA | ACG |
| rs4785592 | TGGTCATTGTGGGAACG | ACT |
| rs4785593 | GGGACAGGGAGAGAAGG | ACG |
| rs4785711 | GGGATTACAGGCGTGAG | ACT |
| rs4785712 | AGAGTGCATTGAGGCCG | ACT |
| rs4785713 | TCATGTTGGCAGTCCCA | ACT |
| rs4785714 | GGTGAGAGAATCCGTAT | CGT |
| rs4785594 | GAAGTTCCTTTCTGTTCTT | ACT |
| rs3803689 | TCAAAGTGTCCTTGGGC | ACT |
| rs4785715 | CAAGTGAAAGTCTGGCC | ACG |
| rs3764258 | GATTACAGGTGTGAGCA | ACT |
| rs4785716 | GACCTACACACATGTGAA | ACT |
| rs4785717 | GACACGACCTACACACA | ACG |
| rs2077001 | ACGGCCTCACTATGTTG | ACG |
| rs2377049 | CTGGTCTTGAACTGACT | ACT |
| rs2003522 | TCTTGCTCTGTTGCCCA | ACT |
| rs1230 | CGCAAACGCTGAGTGACT | ACG |
| rs1800359 | CAATTCTCATGTCCCCCAC | ACT |
| rs1061646 | GGTCTGCAACACCAAGAA | ACT |
| rs1800358 | CCAGTCCGGGTTGGGTGC | ACG |
| rs2286392 | TCAGAACGCCAGGATGC | ACG |
| rs2074904 | CCTCCGCTGCCCCAGCC | ACT |
| rs2074903 | CCATGAGTGTGGGTAATAAACT | ACT |

Genetic Analysis

Allelotyping results are shown for female (F) and male (M) cases and controls in Table 24 and Table 25, respectively. Allele frequency is noted in the fourth and fifth columns for melanoma pools and control pools, respectively.

TABLE 24
Females

| | | | | | | | | Melanoma |
|---|---|---|---|---|---|---|---|---|
| | Chromo- | Chromosome | | Female | Female | F | Odds | Associated |
| dbSNP rs# | some | Position | Alleles | Case AF | Control AF | p-Value | Ratio | Allele |
| rs460879 | 16 | 89456684 | T/C | T = 0.492 | T = 0.595 | 0.037 | 1.520 | C |
| | | | | C = 0.508 | C = 0.405 | | | |
| rs460984 | 16 | 89456969 | T/C | T = 0.780 | T = 0.846 | 0.043 | 1.552 | C |
| | | | | C = 0.219 | C = 0.153 | | | |
| rs2437957 | 16 | 89459443 | C/T | C = 0.501 | C = 0.508 | 0.849 | 1.026 | T |
| | | | | T = 0.498 | T = 0.491 | | | |
| rs3815949 | 16 | 89459711 | C/G | C = 0.002 | C = 0.043 | 0.001 | 18.006 | G |
| | | | | G = 0.998 | G = 0.957 | | | |
| rs2437956 | 16 | 89460046 | G/A | G = 0.754 | G = 0.756 | 0.962 | 1.009 | A |
| | | | | A = 0.245 | A = 0.243 | | | |
| rs2437955 | 16 | 89460070 | G/T | G = 0.846 | G = 0.753 | 0.016 | 0.554 | G |
| | | | | T = 0.153 | T = 0.246 | | | |
| rs2434872 | 16 | 89460710 | C/G | C = 0.530 | C = 0.546 | 0.620 | 1.069 | G |
| | | | | G = 0.469 | G = 0.453 | | | |
| rs467357 | 16 | 89461192 | G/A | G = 0.649 | G = 0.659 | 0.779 | 1.044 | A |
| | | | | A = 0.350 | A = 0.340 | | | |
| rs258337 | 16 | 89464505 | A/T | A = 0.571 | A = 0.643 | 0.047 | 1.351 | T |
| | | | | T = 0.428 | T = 0.356 | | | |
| rs258336 | 16 | 89464626 | T/G | T = 0.618 | T = 0.555 | 0.093 | 0.771 | T |
| | | | | G = 0.381 | G = 0.444 | | | |
| rs258335 | 16 | 89464739 | A/G | A = 0.769 | A = 0.752 | 0.602 | 0.911 | A |
| | | | | G = 0.230 | G = 0.247 | | | |
| rs164752 | 16 | 89466185 | G/A | G = 0.128 | G = 0.057 | 0.003 | 0.411 | G |
| | | | | A = 0.871 | A = 0.942 | | | |
| rs154663 | 16 | 89469830 | T/C | T = 0.196 | T = 0.208 | 0.679 | 1.082 | C |
| | | | | C = 0.803 | C = 0.791 | | | |
| rs164753 | 16 | 89471390 | T/C | T = 0.864 | T = 0.832 | 0.219 | 0.780 | T |
| | | | | C = 0.135 | C = 0.167 | | | |
| rs258332 | 16 | 89471478 | G/A | G = 0.950 | G = 0.941 | 0.681 | 0.840 | G |
| | | | | A = 0.050 | A = 0.059 | | | |
| rs2010623 | 16 | 89472820 | C/T | C = 0.961 | C = 0.978 | 0.473 | 1.810 | T |
| | | | | T = 0.039 | T = 0.022 | | | |
| rs187283 | 16 | 89473131 | G/A | G = 0.955 | G = 0.973 | 0.470 | 1.690 | A |
| | | | | A = 0.045 | A = 0.027 | | | |
| rs258330 | 16 | 89473369 | T/G | T = 0.583 | T = 0.626 | 0.244 | 1.199 | G |
| | | | | G = 0.416 | G = 0.373 | | | |
| rs258328 | 16 | 89474109 | C/T | C = 0.870 | C = 0.906 | 0.340 | 1.430 | T |
| | | | | T = 0.130 | T = 0.094 | | | |
| rs459920 | 16 | 89474622 | T/C | T = 0.723 | T = 0.687 | 0.254 | 0.841 | T |
| | | | | C = 0.276 | C = 0.312 | | | |
| rs166297 | 16 | 89474980 | G/A | G = 0.920 | G = 0.908 | 0.574 | 0.864 | G |
| | | | | A = 0.079 | A = 0.091 | | | |
| rs258318 | 16 | 89475845 | T/C | T = 0.204 | T = 0.250 | 0.116 | 1.300 | C |
| | | | | C = 0.795 | C = 0.749 | | | |
| rs258317 | 16 | 89476033 | C/T | C = 0.978 | C = 0.981 | 0.853 | 1.123 | T |
| | | | | T = 0.022 | T = 0.019 | | | |
| rs171805 | 16 | 89477409 | G/A | G = 0.979 | G = 0.954 | 0.239 | 0.450 | G |
| | | | | A = 0.021 | A = 0.046 | | | |
| rs2081984 | 16 | 89477721 | C/T | C = 0.874 | C = 0.870 | 0.830 | 0.958 | C |
| | | | | T = 0.125 | T = 0.129 | | | |
| rs464586 | 16 | 89477883 | C/T | C = 0.354 | C = 0.157 | ˜0.001 | 0.340 | C |
| | | | | T = 0.645 | T = 0.842 | | | |
| rs460319 | 16 | 89477888 | G/T | G = 0.166 | G = 0.213 | 0.103 | 1.362 | T |
| | | | | T = 0.833 | T = 0.786 | | | |
| rs447735 | 16 | 89478144 | A/G | A = 0.699 | A = 0.660 | 0.224 | 0.833 | A |
| | | | | G = 0.300 | G = 0.339 | | | |
| rs2377058 | 16 | 89478626 | T/C | T = 0.780 | T = 0.796 | 0.609 | 1.100 | C |
| | | | | C = 0.220 | C = 0.204 | | | |
| rs604868 | 16 | 89479972 | G/A | G = 0.118 | G = 0.116 | 0.912 | 0.976 | G |
| | | | | A = 0.881 | A = 0.883 | | | |
| rs2118193 | 16 | 89483698 | C/T | C = 0.814 | C = | | | |
| | | | | T = 0.186 | T = | | | |
| rs467035 | 16 | 89484080 | C/G | C = 0.647 | C = 0.632 | 0.653 | 0.937 | C |
| | | | | G = 0.352 | G = 0.367 | | | |
| rs2115401 | 16 | 89484404 | C/T | C = 0.660 | C = 0.660 | 0.999 | 1.000 | C |
| | | | | T = 0.339 | T = 0.339 | | | |
| rs417323 | 16 | 89490072 | T/G | T = 0.634 | T = 0.641 | 0.877 | 1.027 | G |
| | | | | G = 0.365 | G = 0.358 | | | |
| rs2377233 | 16 | 89490697 | C/G | C = 0.891 | C = 0.845 | 0.110 | 0.668 | C |
| | | | | G = 0.108 | G = 0.154 | | | |
| AA | 16 | 89493039 | G/C | G = 0.077 | G = 0.042 | 0.052 | 0.535 | G |
| ds000175 | | | | C = 0.922 | C = 0.957 | | | |
| rs9746114 | 16 | 89495122 | C/G | C = 0.953 | C = 0.948 | 0.763 | 0.897 | C |
| | | | | G = 0.046 | G = 0.051 | | | |
| rs3751700 | 16 | 89495989 | G/A | G = 0.974 | G = 0.953 | 0.524 | 0.544 | G |
| | | | | A = 0.025 | A = 0.046 | | | |
| rs2277905 | 16 | 89496296 | A/G | A = 0.919 | A = 0.886 | 0.160 | 0.687 | A |
| | | | | G = 0.080 | G = 0.113 | | | |
| rs397891 | 16 | 89496826 | C/G | C = 0.695 | C = | | | |
| | | | | G = 0.305 | G = | | | |
| rs3794638 | 16 | 89497231 | G/T | G = 0.924 | G = 0.901 | 0.489 | 0.751 | G |
| | | | | T = 0.075 | T = 0.098 | | | |
| rs3794637 | 16 | 89497365 | G/A | G = | G = | | | |
| | | | | A = | A = | | | |
| rs258324 | 16 | 89498050 | C/A | C = 0.897 | C = 0.891 | 0.777 | 0.934 | C |
| | | | | A = 0.102 | A = 0.108 | | | |
| rs258323 | 16 | 89498489 | C/G | C = 0.823 | C = 0.779 | 0.225 | 0.760 | C |
| | | | | G = 0.176 | G = 0.220 | | | |
| rs2075880 | 16 | 89499011 | A/G | A = 0.623 | A = 0.695 | 0.027 | 1.380 | G |
| | | | | G = 0.376 | G = 0.304 | | | |
| rs258322 | 16 | 89499698 | C/T | C = 0.909 | C = 0.950 | 0.090 | 1.903 | T |
| | | | | T = 0.090 | T = 0.049 | | | |
| rs258321 | 16 | 89500268 | C/T | C = | C = | | | |
| | | | | T = | T = | | | |
| rs164744 | 16 | 89500563 | A/C | A = | A = | | | |
| | | | | C = | C = | | | |
| rs3833829 | 16 | 89500576 | A/— | A = 0.103 | A = 0.140 | 0.123 | 1.411 | — |
| | | | | — = 0.896 | — = 0.859 | | | |
| AB | 16 | 89500994 | T/C | T = 0.526 | T = 0.465 | 0.064 | 0.783 | T |
| ds000276 | | | | C = 0.473 | C = 0.534 | | | |
| rs164743 | 16 | 89501226 | A/G | A = 0.161 | A = 0.244 | 0.036 | 1.681 | G |
| | | | | G = 0.838 | G = 0.755 | | | |
| rs164742 | 16 | 89501520 | C/T | C = 0.652 | C = 0.700 | 0.128 | 1.247 | T |
| | | | | T = 0.347 | T = 0.299 | | | |
| rs187282 | 16 | 89502768 | T/C | T = 0.060 | T = 0.036 | 0.226 | 0.588 | T |
| | | | | C = 0.939 | C = 0.963 | | | |
| rs465507 | 16 | 89504287 | C/G | C = 0.194 | C = 0.151 | 0.112 | 0.735 | C |
| | | | | G = 0.805 | G = 0.848 | | | |
| rs2162943 | 16 | 89504447 | G/A | G = 0.796 | G = 0.833 | 0.172 | 1.281 | A |
| | | | | A = 0.203 | A = 0.166 | | | |
| AC | 16 | 89504754 | G/C | G = 0.238 | G = 0.175 | 0.105 | 0.681 | G |
| ds000173 | | | | C = 0.761 | C = 0.824 | | | |
| rs1946482 | 16 | 89506205 | T/C | T = 0.900 | T = 0.892 | 0.714 | 0.915 | T |
| | | | | C = 0.099 | C = 0.107 | | | |
| rs8404 | 16 | 89506442 | C/T | C = 0.227 | C = 0.141 | 0.006 | 0.563 | C |
| | | | | T = 0.772 | T = 0.858 | | | |
| rs1045814 | 16 | 89506710 | G/A | G = 0.659 | G = 0.664 | 0.864 | 1.025 | A |
| | | | | A = 0.340 | A = 0.335 | | | |
| rs4247353 | 16 | 89507009 | T/C | T = 0.376 | T = 0.418 | 0.341 | 1.193 | C |
| | | | | C = 0.623 | C = 0.581 | | | |
| rs462769 | 16 | 89507058 | A/G | A = 0.665 | A = 0.687 | 0.540 | 1.106 | G |
| | | | | G = 0.334 | G = 0.312 | | | |
| rs3803690 | 16 | 89507154 | C/G | C = 0.668 | C = 0.702 | 0.326 | 1.167 | G |
| | | | | G = 0.331 | G = 0.297 | | | |
| rs3751696 | 16 | 89507414 | A/T | A = | A = | | | |
| | | | | T = | T = | | | |
| rs9972864 | 16 | 89508992 | A/G | A = 0.048 | A = 0.050 | 0.872 | 1.058 | G |
| | | | | G = 0.951 | G = 0.949 | | | |
| rs10567226 | 16 | 89509757 | TTT/— | TTT = 0.916 | TTT = 0.930 | 0.430 | 1.233 | — |
| | | | | — = 0.083 | — = 0.069 | | | |
| rs417414 | 16 | 89509943 | A/G | A = 0.297 | A = 0.273 | | 0.886 | A |
| | | | | G = 0.702 | G = 0.726 | | | |
| rs154661 | 16 | 89510366 | G/C | G = 0.045 | G = 0.081 | 0.148 | 1.838 | C |
| | | | | C = 0.954 | C = 0.918 | | | |
| rs4785704 | 16 | 89516165 | G/A | G = 0.058 | G = 0.056 | 0.899 | 0.950 | G |
| | | | | A = 0.942 | A = 0.944 | | | |
| rs4785584 | 16 | 89516179 | C/T | C = 0.875 | C = 0.860 | 0.577 | 0.880 | C |
| | | | | T = 0.125 | T = 0.140 | | | |
| rs4785585 | 16 | 89516200 | C/T | C = 0.797 | C = 0.801 | 0.882 | 1.030 | T |
| | | | | T = 0.203 | T = 0.199 | | | |
| rs4785705 | 16 | 89516209 | T/C | T = 0.513 | T = 0.482 | 0.383 | 0.880 | T |
| | | | | C = 0.487 | C = 0.518 | | | |
| rs4785586 | 16 | 89516211 | G/A | G = | G = | | | |
| | | | | A = | A = | | | |
| rs4785587 | 16 | 89516414 | A/G | A = 0.758 | A = 0.714 | 0.134 | 0.795 | A |
| | | | | G = 0.241 | G = 0.285 | | | |
| rs2016968 | 16 | 89516701 | C/G | C = 0.655 | C = 0.639 | 0.649 | 0.931 | C |
| | | | | G = 0.344 | G = 0.360 | | | |
| rs3751693 | 16 | 89518191 | C/T | C = 0.377 | C = 0.367 | 0.816 | 0.960 | C |
| | | | | T = 0.623 | T = 0.633 | | | |
| rs3809646 | 16 | 89530650 | A/G | A = 0.779 | A = 0.746 | 0.282 | 0.831 | A |
| | | | | G = 0.220 | G = 0.253 | | | |
| rs4785590 | 16 | 89533962 | G/C | G = 0.657 | G = 0.621 | 0.333 | 0.856 | G |
| | | | | C = 0.342 | C = 0.378 | | | |
| rs2099105 | 16 | 89537350 | A/G | A = 0.439 | A = 0.386 | 0.117 | 0.802 | A |
| | | | | G = 0.560 | G = 0.613 | | | |
| rs4785708 | 16 | 89540364 | C/T | C = 0.690 | C = 0.640 | 0.117 | 0.799 | C |
| | | | | T = 0.309 | T = 0.359 | | | |
| rs4785710 | 16 | 89540551 | T/C | T = 0.553 | T = 0.598 | 0.251 | 1.203 | C |
| | | | | C = 0.446 | C = 0.401 | | | |
| rs4785591 | 16 | 89541308 | C/T | C = 0.716 | C = 0.755 | 0.200 | 1.225 | T |
| | | | | T = 0.283 | T = 0.244 | | | |
| rs4785592 | 16 | 89541310 | C/G | C = 0.031 | C = 0.010 | 0.037 | 0.268 | C |
| | | | | G = 0.969 | G = 0.990 | | | |
| rs4785593 | 16 | 89541422 | C/T | C = 0.396 | C = 0.355 | 0.218 | 0.843 | C |
| | | | | T = 0.603 | T = 0.644 | | | |
| rs4785711 | 16 | 89541741 | C/G | C = 0.977 | C = 0.983 | 0.730 | 1.376 | G |
| | | | | G = 0.023 | G = 0.017 | | | |
| rs4785712 | 16 | 89541749 | G/C | G = 0.239 | G = 0.223 | 0.584 | 0.914 | G |
| | | | | C = 0.760 | C = 0.776 | | | |
| rs4785713 | 16 | 89541893 | G/C | G = 0.581 | G = 0.527 | 0.113 | 0.804 | G |
| | | | | C = 0.418 | C = 0.472 | | | |
| rs4785714 | 16 | 89542199 | C/A | C = 0.718 | C = 0.631 | 0.011 | 0.673 | C |
| | | | | A = 0.281 | A = 0.368 | | | |
| rs4785594 | 16 | 89542311 | C/G | C = 0.708 | C = 0.753 | 0.210 | 1.256 | G |
| | | | | G = 0.291 | G = 0.246 | | | |
| rs3803689 | 16 | 89543853 | A/G | A = 0.681 | A = 0.623 | 0.069 | 0.776 | A |
| | | | | G = 0.318 | G = 0.376 | | | |
| rs4785715 | 16 | 89543930 | G/A | G = 0.631 | G = 0.642 | 0.738 | 1.047 | A |
| | | | | A = 0.368 | A = 0.357 | | | |
| rs3764258 | 16 | 89544809 | C/G | C = 0.903 | C = 0.910 | 0.760 | 1.077 | G |
| | | | | G = 0.096 | G = 0.089 | | | |
| rs4785716 | 16 | 89544922 | T/C | T = 0.859 | T = 0.956 | ˜0.001 | 3.627 | C |
| | | | | C = 0.140 | C = 0.043 | | | |
| rs4785717 | 16 | 89544928 | G/A | G = 0.600 | G = 0.571 | 0.367 | 0.887 | G |
| | | | | A = 0.399 | A = 0.428 | | | |
| rs2077001 | 16 | 89545885 | C/T | C = 0.412 | C = 0.460 | 0.160 | 1.217 | T |
| | | | | T = 0.587 | T = 0.539 | | | |
| rs2377049 | 16 | 89546763 | A/C | A = 0.772 | A = 0.792 | 0.541 | 1.127 | C |
| | | | | C = 0.227 | C = 0.207 | | | |
| rs2003522 | 16 | 89547081 | C/G | C = 0.387 | C = 0.345 | 0.224 | 0.833 | C |
| | | | | G = 0.612 | G = 0.654 | | | |
| rs1230 | 16 | 89548650 | C/T | C = 0.645 | C = 0.666 | 0.527 | 1.098 | T |
| | | | | T = 0.354 | T = 0.333 | | | |
| rs1800359 | 16 | 89549056 | A/G | A = 0.705 | A = 0.776 | 0.015 | 1.448 | G |
| | | | | G = 0.294 | G = 0.223 | | | |
| rs1061646 | 16 | 89549772 | T/C | T = 0.387 | T = 0.355 | 0.308 | 0.869 | T |
| | | | | C = 0.612 | C = 0.644 | | | |
| rs1800358 | 16 | 89553114 | C/T | C = 0.199 | C = 0.199 | 0.987 | 1.003 | C |
| | | | | T = 0.800 | T = 0.800 | | | |
| rs2286392 | 16 | 89553344 | C/T | C = 0.266 | C = 0.270 | 0.881 | 1.023 | T |
| | | | | T = 0.733 | T = 0.729 | | | |
| rs2074904 | 16 | 89555341 | T/C | T = 0.914 | T = 0.919 | 0.807 | 1.063 | C |
| | | | | C = 0.085 | C = 0.080 | | | |
| rs2074903 | 16 | 89555458 | A/G | A = 0.921 | A = 0.906 | 0.507 | 0.829 | A |
| | | | | G = 0.078 | G = 0.093 | | | |

TABLE 25
Males

| | | | | | | | | Melanoma |
|---|---|---|---|---|---|---|---|---|
| | Chromo- | Chromosome | | Male | Male | M | Odds | Associated |
| dbSNP rs# | some | Position | Alleles | Case AF | Control AF | p-Value | Ratio | Allele |
| rs460879 | 16 | 89456684 | T/C | T = 0.532 | T = | | | |
| | | | | C = 0.468 | C = | | | |
| rs460984 | 16 | 89456969 | T/C | T = | T = | | | |
| | | | | C = | C = | | | |
| rs2437957 | 16 | 89459443 | C/T | C = 0.541 | C = 0.512 | 0.430 | 0.890 | C |
| | | | | T = 0.458 | T = 0.487 | | | |
| rs3815949 | 16 | 89459711 | C/G | C = 0.015 | C = 0.002 | 0.207 | 0.108 | C |
| | | | | G = 0.985 | G = 0.998 | | | |
| rs2437956 | 16 | 89460046 | G/A | G = 0.780 | G = 0.723 | 0.081 | 0.737 | G |
| | | | | A = 0.219 | A = 0.276 | | | |
| rs2437955 | 16 | 89460070 | G/T | G = 0.886 | G = 0.825 | 0.037 | 0.605 | G |
| | | | | T = 0.113 | T = 0.174 | | | |
| rs2434872 | 16 | 89460710 | C/G | C = 0.544 | C = 0.585 | 0.352 | 1.183 | G |
| | | | | G = 0.455 | G = 0.414 | | | |
| rs467357 | 16 | 89461192 | G/A | G = 0.654 | G = 0.650 | 0.908 | 0.982 | G |
| | | | | A = 0.345 | A = 0.349 | | | |
| rs258337 | 16 | 89464505 | A/T | A = 0.577 | A = 0.654 | 0.028 | 1.385 | T |
| | | | | T = 0.422 | T = 0.345 | | | |
| rs258336 | 16 | 89464626 | T/G | T = 0.588 | T = 0.522 | 0.068 | 0.765 | T |
| | | | | G = 0.411 | G = 0.477 | | | |
| rs258335 | 16 | 89464739 | A/G | A = 0.777 | A = 0.791 | 0.709 | 1.085 | G |
| | | | | G = 0.222 | G = 0.208 | | | |
| rs164752 | 16 | 89466185 | G/A | G = 0.101 | G = 0.054 | 0.036 | 0.511 | G |
| | | | | A = 0.898 | A = 0.945 | | | |
| rs154663 | 16 | 89469830 | T/C | T = 0.172 | T = 0.207 | 0.270 | 1.252 | C |
| | | | | C = 0.827 | C = 0.792 | | | |
| rs164753 | 16 | 89471390 | T/C | T = 0.902 | T = 0.808 | ˜0.001 | 0.454 | T |
| | | | | C = 0.097 | C = 0.191 | | | |
| rs258332 | 16 | 89471478 | G/A | G = 0.952 | G = 0.933 | 0.383 | 0.700 | G |
| | | | | A = 0.048 | A = 0.067 | | | |
| rs2010623 | 16 | 89472820 | C/T | C = 0.971 | C = 0.952 | 0.535 | 0.590 | C |
| | | | | T = 0.029 | T = 0.048 | | | |
| rs187283 | 16 | 89473131 | G/A | G = 0.967 | G = 0.971 | 0.904 | 1.130 | A |
| | | | | A = 0.033 | A = 0.029 | | | |
| rs258330 | 16 | 89473369 | T/G | T = 0.632 | T = 0.606 | 0.456 | 0.895 | T |
| | | | | G = 0.367 | G = 0.393 | | | |
| rs258328 | 16 | 89474109 | C/T | C = 0.855 | C = 0.782 | 0.062 | 0.610 | C |
| | | | | T = 0.145 | T = 0.218 | | | |
| rs459920 | 16 | 89474622 | T/C | T = 0.718 | T = 0.686 | 0.317 | 0.857 | T |
| | | | | C = 0.281 | C = 0.313 | | | |
| rs166297 | 16 | 89474980 | G/A | G = 0.944 | G = 0.916 | 0.171 | 0.643 | G |
| | | | | A = 0.055 | A = 0.083 | | | |
| rs258318 | 16 | 89475845 | T/C | T = 0.149 | T = 0.223 | 0.027 | 1.633 | C |
| | | | | C = 0.850 | C = 0.776 | | | |
| rs258317 | 16 | 89476033 | C/T | C = 0.974 | C = 0.978 | 0.820 | 1.176 | T |
| | | | | T = 0.025 | T = 0.021 | | | |
| rs171805 | 16 | 89477409 | G/A | G = | G = 0.964 | | | |
| | | | | A = | A = 0.036 | | | |
| rs2081984 | 16 | 89477721 | C/T | C = 0.860 | C = 0.845 | 0.552 | 0.887 | C |
| | | | | T = 0.139 | T = 0.154 | | | |
| rs464586 | 16 | 89477883 | C/T | C = 0.304 | C = 0.132 | ˜0.001 | 0.350 | C |
| | | | | T = 0.695 | T = 0.867 | | | |
| rs460319 | 16 | 89477888 | G/T | G = 0.156 | G = 0.230 | 0.010 | 1.617 | T |
| | | | | T = 0.843 | T = 0.769 | | | |
| rs447735 | 16 | 89478144 | A/G | A = 0.686 | A = 0.654 | 0.327 | 0.864 | A |
| | | | | G = 0.313 | G = 0.345 | | | |
| rs2377058 | 16 | 89478626 | T/C | T = 0.840 | T = 0.840 | 0.609 | 1.000 | T |
| | | | | C = 0.160 | C = 0.160 | | | |
| rs604868 | 16 | 89479972 | G/A | G = 0.103 | G = 0.109 | 0.791 | 1.063 | A |
| | | | | A = 0.896 | A = 0.890 | | | |
| rs2118193 | 16 | 89483698 | C/T | C = | C = | | | |
| | | | | T = | T = | | | |
| rs467035 | 16 | 89484080 | C/G | C = 0.662 | C = 0.620 | 0.254 | 0.834 | C |
| | | | | G = 0.337 | G = 0.379 | | | |
| rs2115401 | 16 | 89484404 | C/T | C = 0.672 | C = 0.651 | 0.564 | 0.909 | C |
| | | | | T = 0.327 | T = 0.348 | | | |
| rs417323 | 16 | 89490072 | T/G | T = 0.625 | T = 0.663 | 0.444 | 1.183 | G |
| | | | | G = 0.374 | G = 0.336 | | | |
| rs2377233 | 16 | 89490697 | C/G | C = 0.899 | C = 0.833 | 0.038 | 0.562 | C |
| | | | | G = 0.100 | G = 0.166 | | | |
| AA | 16 | 89493039 | G/C | G = 0.049 | G = 0.065 | 0.405 | 1.335 | C |
| | | | | C = 0.950 | C = 0.934 | | | |
| rs9746114 | 16 | 89495122 | C/G | C = 0.971 | C = 0.942 | 0.166 | 0.471 | C |
| | | | | G = 0.028 | G = 0.057 | | | |
| rs3751700 | 16 | 89495989 | G/A | G = 0.987 | G = 0.941 | 0.142 | 0.212 | G |
| | | | | A = 0.013 | A = 0.059 | | | |
| rs2277905 | 16 | 89496296 | A/G | A = 0.955 | A = 0.934 | 0.332 | 0.663 | A |
| | | | | G = 0.044 | G = 0.065 | | | |
| rs397891 | 16 | 89496826 | C/G | C = 0.608 | C = | | | |
| | | | | G = 0.392 | G = | | | |
| rs3794638 | 16 | 89497231 | G/T | G = | G = | | | |
| | | | | T = | T = | | | |
| rs3794637 | 16 | 89497365 | G/A | G = 0.874 | G = 0.832 | 0.189 | 0.716 | G |
| | | | | A = 0.125 | A = 0.167 | | | |
| rs258324 | 16 | 89498050 | C/A | C = 0.916 | C = 0.893 | 0.334 | 0.770 | C |
| | | | | A = 0.083 | A = 0.106 | | | |
| rs258323 | 16 | 89498489 | C/G | C = 0.816 | C = 0.785 | 0.300 | 0.824 | C |
| | | | | G = 0.183 | G = 0.214 | | | |
| rs2075880 | 16 | 89499011 | A/G | A = 0.596 | A = 0.707 | 0.001 | 1.631 | G |
| | | | | G = 0.403 | G = 0.292 | | | |
| rs258322 | 16 | 89499698 | C/T | C = 0.908 | C = 0.954 | 0.014 | 2.128 | T |
| | | | | T = 0.091 | T = 0.045 | | | |
| rs258321 | 16 | 89500268 | C/T | C = 0.606 | C = 0.735 | 0.003 | 1.801 | T |
| | | | | T = 0.393 | T = 0.264 | | | |
| rs164744 | 16 | 89500563 | A/C | A = 0.715 | A = 0.573 | ˜0.001 | 0.536 | A |
| | | | | C = 0.284 | C = 0.426 | | | |
| rs3833829 | 16 | 89500576 | A/— | A = 0.103 | A = 0.143 | 0.152 | 1.451 | — |
| | | | | — = 0.896 | — = 0.856 | | | |
| AB | 16 | 89500994 | T/C | T = 0.492 | T = 0.425 | 0.078 | 0.764 | T |
| | | | | C = 0.507 | C = 0.574 | | | |
| rs164743 | 16 | 89501226 | A/G | A = | A = | | | |
| | | | | G = | G = | | | |
| rs164742 | 16 | 89501520 | C/T | C = 0.678 | C = 0.760 | 0.015 | 1.501 | T |
| | | | | T = 0.321 | T = 0.239 | | | |
| rs187282 | 16 | 89502768 | T/C | T = 0.066 | T = 0.048 | 0.625 | 0.713 | T |
| | | | | C = 0.933 | C = 0.951 | | | |
| rs465507 | 16 | 89504287 | C/G | C = 0.218 | C = 0.171 | 0.106 | 0.739 | C |
| | | | | G = 0.781 | G = 0.828 | | | |
| rs2162943 | 16 | 89504447 | G/A | G = 0.822 | G = 0.797 | 0.470 | 0.851 | G |
| | | | | A = 0.177 | A = 0.202 | | | |
| AC | 16 | 89504754 | G/C | G = 0.253 | G = 0.219 | 0.325 | 0.826 | G |
| | | | | C = 0.746 | C = 0.780 | | | |
| rs1946482 | 16 | 89506205 | T/C | T = 0.896 | T = 0.878 | 0.512 | 0.836 | T |
| | | | | C = 0.103 | C = 0.121 | | | |
| rs8404 | 16 | 89506442 | C/T | C = 0.195 | C = 0.121 | 0.017 | 0.567 | C |
| | | | | T = 0.804 | T = 0.878 | | | |
| rs1045814 | 16 | 89506710 | G/A | G = 0.637 | G = 0.675 | 0.328 | 1.183 | A |
| | | | | A = 0.362 | A = 0.324 | | | |
| rs4247353 | 16 | 89507009 | T/C | T = | T = | | | |
| | | | | C = | C = | | | |
| rs462769 | 16 | 89507058 | A/G | A = 0.663 | A = 0.719 | 0.103 | 1.300 | G |
| | | | | G = 0.336 | G = 0.280 | | | |
| rs3803690 | 16 | 89507154 | C/G | C = 0.687 | C = 0.690 | 0.931 | 1.016 | G |
| | | | | G = 0.312 | G = 0.309 | | | |
| rs3751696 | 16 | 89507414 | A/T | A = 0.725 | A = 0.698 | 0.480 | 0.877 | A |
| | | | | T = 0.274 | T = 0.301 | | | |
| rs9972864 | 16 | 89508992 | A/G | A = 0.047 | A = 0.049 | 0.930 | 1.038 | G |
| | | | | G = 0.952 | G = 0.950 | | | |
| rs10567226 | 16 | 89509757 | TTT/— | TTT = 0.925 | TTT = 0.936 | 0.609 | 1.185 | — |
| | | | | — = 0.074 | — = 0.063 | | | |
| rs417414 | 16 | 89509943 | A/G | A = 0.287 | A = 0.235 | ˜0.001 | 0.766 | A |
| | | | | G = 0.712 | G = 0.764 | | | |
| rs154661 | 16 | 89510366 | G/C | G = 0.025 | G = 0.017 | 0.728 | 0.655 | G |
| | | | | C = 0.975 | C = 0.983 | | | |
| rs4785704 | 16 | 89516165 | G/A | G = 0.049 | G = 0.045 | 0.855 | 0.920 | G |
| | | | | A = 0.951 | A = 0.955 | | | |
| rs4785584 | 16 | 89516179 | c/t | C = 0.894 | C = 0.869 | 0.336 | 0.780 | C |
| | | | | T = 0.106 | T = 0.131 | | | |
| rs4785585 | 16 | 89516200 | c/t | C = 0.810 | C = 0.811 | 0.977 | 1.010 | T |
| | | | | T = 0.190 | T = 0.189 | | | |
| rs4785705 | 16 | 89516209 | T/C | T = 0.500 | T = 0.481 | 0.621 | 0.930 | T |
| | | | | C = 0.500 | C = 0.519 | | | |
| rs4785586 | 16 | 89516211 | G/A | G = 0.804 | G = 0.852 | 0.163 | 1.408 | A |
| | | | | A = 0.195 | A = 0.147 | | | |
| rs4785587 | 16 | 89516414 | A/G | A = 0.742 | A = 0.689 | 0.097 | 0.770 | A |
| | | | | G = 0.257 | G = 0.310 | | | |
| rs2016968 | 16 | 89516701 | C/G | C = 0.667 | C = 0.635 | 0.376 | 0.865 | C |
| | | | | G = 0.332 | G = 0.364 | | | |
| rs3751693 | 16 | 89518191 | C/T | C = 0.390 | C = 0.382 | 0.853 | 0.970 | C |
| | | | | T = 0.610 | T = 0.618 | | | |
| rs3809646 | 16 | 89530650 | A/G | A = 0.786 | A = 0.739 | 0.197 | 0.769 | A |
| | | | | G = 0.213 | G = 0.260 | | | |
| rs4785590 | 16 | 89533962 | G/C | G = 0.643 | G = 0.605 | 0.280 | 0.852 | G |
| | | | | C = 0.356 | C = 0.394 | | | |
| rs2099105 | 16 | 89537350 | A/G | A = 0.420 | A = 0.365 | 0.112 | 0.794 | A |
| | | | | G = 0.579 | G = 0.634 | | | |
| rs4785708 | 16 | 89540364 | C/T | C = 0.674 | C = 0.600 | 0.025 | 0.725 | C |
| | | | | T = 0.325 | T = 0.399 | | | |
| rs4785710 | 16 | 89540551 | T/C | T = 0.572 | T = 0.620 | 0.175 | 1.223 | C |
| | | | | C = 0.427 | C = 0.379 | | | |
| rs4785591 | 16 | 89541308 | C/T | C = 0.767 | C = 0.768 | 0.954 | 1.010 | T |
| | | | | T = 0.232 | T = 0.231 | | | |
| rs4785592 | 16 | 89541310 | C/G | C = 0.022 | C = 0.003 | 0.090 | 0.154 | C |
| | | | | G = 0.978 | G = 0.997 | | | |
| rs4785593 | 16 | 89541422 | C/T | C = 0.381 | C = 0.340 | 0.221 | 0.839 | C |
| | | | | T = 0.618 | T = 0.659 | | | |
| rs4785711 | 16 | 89541741 | C/G | C = 0.982 | C = 0.990 | 0.527 | 1.721 | G |
| | | | | G = 0.018 | G = 0.010 | | | |
| rs4785712 | 16 | 89541749 | G/C | G = 0.233 | G = 0.186 | 0.129 | 0.756 | G |
| | | | | C = 0.766 | C = 0.813 | | | |
| rs4785713 | 16 | 89541893 | G/C | G = 0.548 | G = 0.483 | 0.061 | 0.772 | G |
| | | | | C = 0.451 | C = 0.516 | | | |
| rs4785714 | 16 | 89542199 | C/A | C = 0.659 | C = 0.599 | 0.070 | 0.772 | C |
| | | | | A = 0.340 | A = 0.400 | | | |
| rs4785594 | 16 | 89542311 | C/G | C = 0.729 | C = 0.752 | 0.491 | 1.129 | G |
| | | | | G = 0.270 | G = 0.247 | | | |
| rs3803689 | 16 | 89543853 | A/G | A = 0.637 | A = 0.614 | 0.526 | 0.906 | A |
| | | | | G = 0.362 | G = 0.385 | | | |
| rs4785715 | 16 | 89543930 | G/A | G = 0.641 | G = 0.658 | 0.625 | 1.074 | A |
| | | | | A = 0.358 | A = 0.341 | | | |
| rs3764258 | 16 | 89544809 | C/G | C = 0.917 | C = 0.912 | 0.804 | 0.936 | C |
| | | | | G = 0.082 | G = 0.087 | | | |
| rs4785716 | 16 | 89544922 | T/C | T = | T = | | | |
| | | | | C = | C = | | | |
| rs4785717 | 16 | 89544928 | G/A | G = 0.599 | G = 0.539 | 0.075 | 0.780 | G |
| | | | | A = 0.400 | A = 0.460 | | | |
| rs2077001 | 16 | 89545885 | C/T | C = 0.461 | C = 0.474 | 0.703 | 1.056 | T |
| | | | | T = 0.538 | T = 0.525 | | | |
| rs2377049 | 16 | 89546763 | A/C | A = 0.777 | A = 0.800 | 0.467 | 1.149 | C |
| | | | | C = 0.222 | C = 0.199 | | | |
| rs2003522 | 16 | 89547081 | C/G | C = 0.355 | C = 0.315 | 0.309 | 0.837 | C |
| | | | | G = 0.644 | G = 0.684 | | | |
| rs1230 | 16 | 89548650 | C/T | C = 0.663 | C = 0.672 | 0.774 | 1.043 | T |
| | | | | T = 0.336 | T = 0.327 | | | |
| rs1800359 | 16 | 89549056 | A/G | A = 0.755 | A = 0.805 | 0.085 | 1.340 | G |
| | | | | G = 0.244 | G = 0.194 | | | |
| rs1061646 | 16 | 89549772 | T/C | T = 0.375 | T = 0.332 | 0.261 | 0.830 | T |
| | | | | C = 0.624 | C = 0.667 | | | |
| rs1800358 | 16 | 89553114 | C/T | C = 0.204 | C = 0.176 | 0.362 | 0.835 | C |
| | | | | T = 0.795 | T = 0.823 | | | |
| rs2286392 | 16 | 89553344 | C/T | C = 0.268 | C = 0.286 | 0.582 | 1.094 | T |
| | | | | T = 0.731 | T = 0.713 | | | |
| rs2074904 | 16 | 89555341 | T/C | T = 0.931 | T = 0.919 | 0.537 | 0.839 | T |
| | | | | C = 0.068 | C = 0.080 | | | |
| rs2074903 | 16 | 89555458 | A/G | A = 0.915 | A = 0.914 | 0.943 | 0.981 | A |
| | | | | G = 0.084 | G = 0.085 | | | |

Allelotyping results were considered significant with a calculated p-value of less than or equal to 0.05 for allelotype results. These values are indicated in bold. The assay failed for those SNPs in which the allele frequency is blank. The combined allelotyping p-values for males and females were plotted in FIG. 3A and separately for females and males in FIGS. 3B and 3C, respectively. The position of each SNP on the chromosome is presented on the x-axis. The y-axis gives the negative logarithm (base 10) of the p-value comparing the estimated allele frequency in the case group to that of the control group. The minor allele frequency of the control group for each SNP designated by an X or other symbol on the graphs in FIGS. 3A, 3B and 3C can be determined by consulting Table 24 or 25. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brooks/Cole.). The black line (or generally top-most curve, e.g., see peak in left-most graph just to the left of position 92150000) provides a local test for excess statistical significance to identify regions of association. This was created by use of a 10 kb sliding window with 1 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 10⁻⁸ were truncated at that value.

Finally, the exons and introns of the genes in the covered region are plotted below each graph at the appropriate chromosomal positions. The gene boundary is indicated by the broken horizontal line. The exon positions are shown as thick, unbroken bars. An arrow is place at the 3′ end of each gene to show the direction of transcription.

### Example 8

### PCLO Proximal SNPs

It has been discovered that a polymorphic variation (rs1044639) in the untranslated region of a gene encoding presynaptic cytomatrix protein (PCLO) is associated with the occurrence of melanoma. See Table 3. Subsequently, seventy-six allelic variants located within or nearby the target gene were identified and subsequently allelotyped in melanoma case and control sample sets as described in Examples 1 and 2. Several SNPs, designated by SNP_ID “AD”, “AB”, and “AF” in column 1 of Table 26, are novel and were identified through SNP discovery methods using dye terminator sequencing. Methods of diedeoxynucleotide chain termination chemistry derived sequencing are described by Sanger et al. (Proc. Nat. Acad. Sci. USA 74, 5463-5467, 1997). Briefly, terminators are labeled with fluorescent dyes for automated detection. Fluorescein is measured by an argon ion laser in the sequencing instrument, which transfers light energy to a rhodamine acceptor dye. Each acceptor dye emits light at a characteristic wavelength for detection, identifying the nucleotide that terminated extension of the DNA fragment. Novel SNPs are identified when sequence from multiple samples are compared on one or more strands. The forward and reverse primers used to identify AD are CGTGCCTGATGGGATATACA and TTGGCTCAAAGGCAGCTAAT, respectively. The forward and reverse primers used to identify AE are TGCAACAACAAAGAGAACCA and TTCTCAGAGCCTTGTAGACTTCC, respectively. The forward and reverse primers used to identify AF are GTGAACTCATGCAGCAAGGA and GGTGGAACCTTTGCCAGTTA, respectively. The polymorphic variants are set forth in Table 26. The chromosome position provided in column four of Table 26 is based on Genome “Build 34” of NCBI's GenBank and dbSNP build “119”.

TABLE 26

| | | | | | Allele | |
|---|---|---|---|---|---|---|
| | Chromo- | Position in | Chromosome | Allele | Present in | IUPAC |
| dbSNP rs# | some | SEQ ID NO: 6 | Position | Variants | SEQ ID NO: 6 | Code |
| rs2158220 | 7 | 220 | 82023020 | T/C | T | Y |
| rs1024465 | 7 | 1,365 | 82024165 | G/A | A | R |
| rs4732483 | 7 | 5,798 | 82028598 | T/C | C | Y |
| rs4732485 | 7 | 8,877 | 82031677 | C/T | T | Y |
| rs1859176 | 7 | 17,309 | 82040109 | C/T | C | Y |
| rs1859177 | 7 | 17,446 | 82040246 | C/T | C | Y |
| rs2074401 | 7 | 18,837 | 82041637 | A/G | A | R |
| rs4299948 | 7 | 19,159 | 82041959 | T/G | G | K |
| rs4348435 | 7 | 20,902 | 82043702 | G/A | A | R |
| rs4348436 | 7 | 20,923 | 82043723 | G/C | C | S |
| rs4413718 | 7 | 21,066 | 82043866 | G/C | G | S |
| rs985199 | 7 | 21,147 | 82043947 | G/A | G | R |
| rs7791413 | 7 | 29,008 | 82051808 | T/C | T | Y |
| rs2522830 | 7 | 32,354 | 82055154 | G/A | G | R |
| rs2214414 | 7 | 33,363 | 82056163 | T/C | C | Y |
| rs2189246 | 7 | 33,623 | 82056423 | A/G | G | R |
| rs5885312 | 7 | 33,669 | 82056469 | CTTAAGAA/— | T | N |
| rs3064840 | 7 | 33,672 | 82056472 | AAGAACTT/— | T | N |
| rs2051791 | 7 | 33,989 | 82056789 | C/T | T | Y |
| rs6467914 | 7 | 34,596 | 82057396 | C/T | T | Y |
| rs2214415 | 7 | 34,733 | 82057533 | G/A | A | R |
| rs2189247 | 7 | 36,496 | 82059296 | C/A | C | M |
| rs3064869 | 7 | 36,669 | 82059469 | —/ACA | C | N |
| rs2522831 | 7 | 37,351 | 82060151 | T/C | T | Y |
| rs2715147 | 7 | 37,656 | 82060456 | T/C | C | Y |
| rs3064870 | 7 | 38,055 | 82060855 | —/TAAG | G | N |
| rs2715148 | 7 | 39,286 | 82062086 | A/C | A | M |
| rs2522832 | 7 | 39,615 | 82062415 | A/G | A | R |
| rs1044639 | 7 | 40,809 | 82063609 | G/T | T | K |
| rs2715149 | 7 | 42,063 | 82064863 | A/T | A | W |
| rs2522833 | 7 | 42,959 | 82065759 | C/A | A | M |
| rs2247523 | 7 | 43,655 | 82066455 | C/G | C | S |
| rs2371214 | 7 | 43,923 | 82066723 | T/C | T | Y |
| rs2189248 | 7 | 44,556 | 82067356 | T/C | T | Y |
| rs2715150 | 7 | 45,146 | 82067946 | A/G | A | R |
| rs2715151 | 7 | 45,914 | 82068714 | C/G | C | S |
| rs2371215 | 7 | 46,745 | 82069545 | T/A | T | W |
| rs2715152 | 7 | 46,917 | 82069717 | G/A | G | R |
| rs2715153 | 7 | 47,109 | 82069909 | T/C | C | Y |
| rs972346 | 7 | 49,786 | 82072586 | C/T | C | Y |
| rs2715154 | 7 | 50,117 | 82072917 | G/T | G | K |
| rs2522834 | 7 | 50,197 | 82072997 | T/C | C | Y |
| rs2023847 | 7 | 50,544 | 82073344 | G/T | T | K |
| rs2715155 | 7 | 51,305 | 82074105 | A/G | A | R |
| rs2715156 | 7 | 52,085 | 82074885 | A/G | G | R |
| rs10715043 | 7 | 53,523 | 82076323 | —/G | G | N |
| AD | 7 | 53,843 | 82076643 | C/G | C | S |
| rs2301722 | 7 | 54,485 | 82077285 | A/C | A | M |
| rs2257207 | 7 | 57,435 | 82080235 | A/T | A | W |
| rs2715157 | 7 | 57,625 | 82080425 | A/G | A | R |
| rs2715158 | 7 | 58,857 | 82081657 | G/C | G | S |
| rs2715159 | 7 | 60,659 | 82083459 | A/G | A | R |
| rs2522835 | 7 | 60,747 | 82083547 | A/T | T | W |
| rs2522836 | 7 | 62,304 | 82085104 | C/A | C | M |
| AE | 7 | 63,624 | 82086424 | T/G | T | K |
| rs2522837 | 7 | 64,633 | 82087433 | A/G | A | R |
| rs2522838 | 7 | 64,808 | 82087608 | A/T | T | W |
| AF | 7 | 65,039 | 82087839 | AT/— | A | N |
| rs2522839 | 7 | 66,899 | 82089699 | G/A | A | R |
| rs2522840 | 7 | 67,666 | 82090466 | G/T | T | K |
| rs2522841 | 7 | 68,445 | 82091245 | T/G | G | K |
| rs10555175 | 7 | 68,577 | 82091377 | —/TTAT | T | N |
| rs10592258 | 7 | 68,581 | 82091381 | —/TATTATT | T | N |
| rs2522842 | 7 | 68,913 | 82091713 | G/T | G | K |
| rs6956848 | 7 | 68,950 | 82091750 | G/T | T | K |
| rs2522843 | 7 | 68,973 | 82091773 | C/A | C | M |
| rs2522844 | 7 | 69,343 | 82092143 | T/C | C | Y |
| rs2522845 | 7 | 71,160 | 82093960 | T/G | T | K |
| rs2715161 | 7 | 71,532 | 82094332 | C/T | C | Y |
| rs6972720 | 7 | 71,926 | 82094726 | C/T | T | Y |
| rs4377905 | 7 | 83,562 | 82106362 | A/G | A | R |
| rs4579453 | 7 | 83,972 | 82106772 | T/C | T | Y |
| rs4260846 | 7 | 84,087 | 82106887 | C/A | C | M |
| rs1986481 | 7 | 85,934 | 82108734 | A/T | A | W |
| rs4628206 | 7 | 86,594 | 82109394 | A/T | T | W |
| rs2888017 | 7 | 89,454 | 82112254 | A/G | G | R |

Assay for Verifying and Allelotyping SNPs

The methods used to verify and allelotype the seventy-six proximal SNPs of Table 26 are the same methods described in Examples 1 and 2 herein. The primers and probes used in these assays are provided in Table 27 and Table 28, respectively.

TABLE 27

| | | |
|---|---|---|
| dbSNP rs # | Forward PCR primer | Reverse PCR primer |

| | | |
|---|---|---|
| rs2158220 | GGGCAGGAGAGAGATTCTAT | CCACAGCCCTTATTACAGTC |
| rs1024465 | AGAGATTGTGTTAAGGCATC | CAAGCAAAACTCTTCTAAAG |
| rs4732483 | CCCACCAAGACTGTAAAAGC | CAGGAAACAACAGATGCTGG |
| rs4732485 | AAGTTCTTCCTGACAGCTTC | AGGGAGTAGTGCAAGATAGG |
| rs1859176 | GCTTATAGTGATTCATGCCC | GGACAAGTTGTTTTTCACTG |
| rs1859177 | CTACCAAAAGGGCACTTGAC | GATTAGTTATTGAGCAGAGGG |
| rs2074401 | CCAGCATTTGTCAGATGAGG | GGTTTAGGTTTCAATTCTGG |
| rs4299948 | GAGAGAGGTTGGAATGACAC | GAGGGAGACTCCATCTCAAA |
| rs4348435 | CTAAGGCTACTACGAGCATG | AAGGGTTAACAGAGTGCCTG |
| rs4348436 | TTTTGGTTGAATTCTAAGGC | AAGGGTTAACAGAGTGCCTG |
| rs4413718 | TGTAGCTTTTTCTCTAGCCG | GAGGACTGTAAGCTTAGAATG |
| rs985199 | TTCCATCTTTTTAGAACGCC | GAGAAAAAGCTACATGGCAC |
| rs7791413 | CCACTCTTTCCTTTACTTGG | TAAGATACATCTTTGGGGCC |
| rs2522830 | AATGCCAAAAGGATATTTGC | TGTATGTAAAAATGATGACC |
| rs2214414 | TTTCAGGGATCTGCAAGGTC | AAAACTCCACTGTACACTCG |
| rs2189246 | GACAGTCACCTTAACAATTAC | GAAACAAGAAGCTTAGAGAC |
| rs5885312 | GAGTTGTGAAGTTCTGAGTG | TCAGCTTTCTTGAAGAGATTC |
| rs3064840 | CTAGAGTTGTGAAGTTCTGAG | CAGCTTTCTTGAAGAGATTC |
| rs2051791 | GTCATTGGCAATGAGGATGC | CAACTACCTAATTCAAATGTC |
| rs6467914 | GAAAATTCATGTGTTTTTGGC | GTATACTGAACAAGACCATG |
| rs2214415 | TTCCACTTTTCATCCACATG | TCTTCCCCCAGATCATGTTG |
| rs2189247 | TCAAACAAAAATAGCTCAGG | GAAGCATTCCTAAAACCTTG |
| rs3064869 | ACCAAACCAACTAGCATGGG | GCCTAAACAACTCTCCGTAG |
| rs2522831 | GTACAGAGTGTTTTGTCATCC | TGCTCACGCATAAAACAGGG |
| rs2715147 | GATCCATGTATATTTCCAGGC | CTAGTTTTTCTCTCTGCACTT |
| rs3064870 | CTCTCACCTTTTCTTCCATC | GGGAAGTGTTTGATTACTGTG |
| rs2715148 | TACCAGTTGGAATGTGGATC | CAAGAGAGAACTTTCACCATG |
| rs2522832 | AATGCCATGCCCACAAAAGC | AGCACACAGCAAGAACATCC |
| rs1044639 | CTCCTTTGTTTCCACCATCC | CGCAAACAAAAAGGACACAC |
| rs2715149 | AAACTGAGCTACTGCCTCTG | GTTCAGTATATCTAGGTAAGG |
| rs2522833 | TACCACCTTGGAGTGTTATC | TTCCTTGGTTTATATCCTTG |
| rs2247523 | GGGACAAAATGGAAAAGAATG | GGTAGTCATATCCCTATTTC |
| rs2371214 | TTGATCCTAGGCAGGAGTAC | GAAGGACTTCAGTAAGTCAC |
| rs2189248 | TACACTTACTATGTACTTGT | AGATTAAAAATCACAAGATC |
| rs2715150 | ACCATAGCAGCAAATAAGGG | ACACTGGAGGTGACAGTTTG |
| rs2715151 | TAATTTTTGCCTACAGTTAC | GGCATTGTGGTGCTAATCTC |
| rs2371215 | TAAGTTGAATGCACAGTGAC | TAGAACACCTACAAGCATTTC |
| rs2715152 | CATCTTTTTCTCAGTACACTC | GCCCCATAAACAATAATTTGG |
| rs2715153 | AATGCAGATGTCCCAAGTTC | ATAAATTTTTGGTTTATGTC |
| rs972346 | GGTGAGAAAACTTGAAGCTC | CCTATGTTTGGATCCTGGTC |
| rs2715154 | GCGTAATTGTTCCACAACAC | CATCGTGAACAAGTTAGGCC |
| rs2522834 | TGGAGAATCAAACTCATTAC | TGGCCAGATACATTTAGAAA |
| rs2023847 | CAGCCTGACTTTATGCTCAC | CAAAATCTGTGGAGTTGAAGG |
| rs2715155 | GAAGGCTTCTTTGGTGTATC | GTTTAGTAAAGGAAAACCAG |
| rs2715156 | ATTGATCAGAGGGTGGGAAG | ATAAGAGTCCTGTTAGCTAG |
| rs10715043 | AGGGACCAAGTTCAAACACC | CTGCACTCCAGCCTCAAAAA |
| AD | TATACACCTGCACTGGACAT | GATTTAAGTTCTCGAGTCTTC |
| rs2301722 | AGGCAGCTAATAAGCTACCC | TTCCAAGGCCTTTGCTTGTC |
| rs2257207 | GTACCGAAAGGTATCTATAGG | GCTGTCAGCACTGAAATGTA |
| rs2715157 | GCACAATTGTGGCTGAATAC | GAAGAAAAAAATGCTTAACTC |
| rs2715158 | AAGCAGAAGTGGAGTGTGAG | AGGTCATCAGGGTAGTACTG |
| rs2715159 | CCACAAAAGACCCTAATAGC | AGTAGCATAATTCCTCTGGC |
| rs2522835 | CCATTCCATATGTCTGTCTG | AGCCAGAGGAATTATGCTAC |
| rs2522836 | ACAGAGTCTCACTCTGTCCC | TCACTTGAATATGGGAGGCG |
| AE | GAGACATTTTTCAAGTTCCTC | CTCTTCTGAGTACAAAACTG |
| rs2522837 | AAACATAGGAAAAAGTGATG | CATGATGAGATAGATTTAAC |
| rs2522838 | CCTGTGAAAGTAATGCTTGG | CTAAAAAGAATCAAGTACGC |
| AF | TGCAGAGGCAGATGTAATTG | GCTACAATGCCCTAGATTTTG |
| rs2522839 | TACACCACTGCACTCCAGTC | CCACAATTATGTTGTTGAACC |
| rs2522840 | TGTGCTCCTGCAGAAATGTG | GGAGGAAGAGTACATTAATA |
| rs2522841 | CTGGAAAATATTACCCATGTG | TCTGTGTACTTTCATACCTG |
| rs10555175 | ATAGTGCAAGACCCTGTCTC | TGAAAGTACACAGATGAGAC |
| rs10592258 | ATAGTGCAAGACCCTGTCTC | TGAAAGTACACAGATGAGAC |
| rs2522842 | AATCCTATCATCGATGCTAC | CCATGCCTGGCCTTTAATAG |
| rs6956848 | CTTTTTCAGTGTTTAGTAGCC | GAGTTTTGCTGAATATGTAGC |
| rs2522843 | TCGATGATAGGATTTGTTGC | TACTAAGTTCTTGAATTACC |
| rs2522844 | CCAATTGTTTTCTAGTAGACT | TGGTAGGCACTACACAATTC |
| rs2522845 | ATGTACACCAGTGGTTTGCC | AAACCAGTAGTGGATCAGCC |
| rs2715161 | TAGCTCCTACATATGGAGTG | AACAGGGCTGGAATTGGAGG |
| rs6972720 | TCTCCAGATCCTATTCTATG | CACAACTCATGTACATCAGG |
| rs4377905 | AATGCAAACTGTCTAAGCAC | TATGGCTGTATCAATATCAC |
| rs4579453 | TCTCAGCTCACTGCACTGTC | GACTGTAATCCCAGCTACTC |
| rs4260846 | AGAGCAAGATGCCGTCTCAG | GGAAATATACCATATAAATG |
| rs1986481 | AAGCATGACATTATGTTTCC | AGTACCCAGGTGTTCTTAGC |
| rs4628206 | GCGTTAGGTACACATGACAC | CTTCACAGAAGAGAGTTTCC |
| rs2888017 | CACTAGTCATCAGGGAAATC | CATCTCCTTGCCAGCATCTC |

TABLE 28

Term

| | | |
|---|---|---|
| dbSNP rs # | Extend Primer | Mix |

| | | |
|---|---|---|
| rs2158220 | CTGAGGCCTGCCCCTGA | ACT |
| rs1024465 | TTAAGGCATCTCTGTATATACTAA | ACG |
| rs4732483 | CAGATGCTGGAGAGGAT | ACT |
| rs4732485 | GACAGCTTCAGATATTTCA | ACG |
| rs1859176 | CATGCCCAATTCATATAGATA | ACG |
| rs1859177 | TCTGTCTTGTTCCCATC | ACG |
| rs2074401 | CAGATGAGGTTTCTGAAG | ACT |
| rs4299948 | AAGGACTATAGGGCTACTA | ACT |
| rs4348435 | CATGCCTACCTGTTAATATG | ACG |
| rs4348436 | GAATTCTAAGGCTACTACGA | ACT |
| rs4413718 | AAGAAGTGGTCTACTGTC | ACT |
| rs985199 | GAACGCCTTATCTAAGCA | ACG |
| rs7791413 | CTTGGTACTTAGTAATACACTTA | ACT |
| rs2522830 | AGGATATTTGCAAACGTG | ACG |
| rs2214414 | TGCAAGGTCAAAATATTCTT | ACT |
| rs2189246 | ATCAAGTTTTCAGCTTTCTTGAA | ACT |
| rs5885312 | GTGAAGTTCTGAGTGAAACAAG | CGT |
| rs3064840 | GTGAAGTTCTGAGTGAAACAAG | CGT |
| rs2051791 | GATGCTTTAAAACAAAAACTACA | ACG |
| rs6467914 | ATGTGTTTTTGGCTTTTTAGTGA | ACG |
| rs2214415 | CCATAATATTTCACCCATTCT | ACG |
| rs2189247 | AAAATAGCTCAGGTTTTTCA | CGT |
| rs3064869 | CATGGGGAAAACAACAACAAC | CGT |
| rs2522831 | ACATTAAGTGGATGAGGT | ACT |
| rs2715147 | TATTTCCAGGCACTTCA | ACT |
| rs3064870 | TTCTTCATGTTATTCACGTTCTT | CGT |
| rs2715148 | GAATGTGGATCAGTGTTT | ACT |
| rs2522832 | AGACAATCTCTAAAAATTAA | ACT |
| rs1044639 | CATCCATCCAACCTGGCTC | CGT |
| rs2715149 | CCTCTGTGGGATAAAACA | CGT |
| rs2522833 | GAGTGTTATCGAGGTGAG | CGT |
| rs2247523 | TGAGTAAGTTGCAATTACAAA | ACT |
| rs2371214 | CAGGAGTACTCTAGATTAGT | ACT |
| rs2189248 | CAAATAATCATGAAATTGGTAGC | ACT |
| rs2715150 | AAATAAGGGAAAGGAAGTC | ACT |
| rs2715151 | TTGCCTACAGTTACTTATCT | ACT |
| rs2371215 | GTAGTTTTCTTTGCCCT | CGT |
| rs2715152 | CAATGTTTTAGTTTTGCTTTTC | ACG |
| rs2715153 | TTTGGTTCATCAATTGTAAAATA | ACT |
| rs972346 | GAAGCTCAGAAAAATTAAGG | ACG |
| rs2715154 | TTCCACAACACTCACTT | CGT |
| rs2522834 | CATTTTAACTTTTGTCCAATCA | ACT |
| rs2023847 | TAAGAATCCCCCTGTTT | CGT |
| rs2715155 | GGTGTATCTACTTTCATAAAATT | ACT |
| rs2715156 | AGGGTGGGAAGAAAAAA | ACT |
| rs10715043 | TCAAACACCTATTATCTTTTTTTTT | ACT |
| AD | GCACTGGACATCCAGAAGAC | ACT |
| rs2301722 | GGTCTTTCTTTTAATCACAC | ACT |
| rs2257207 | CGAAAGGTATCTATAGGTTTAATA | CGT |
| rs2715157 | AACAAATATTTGAGAAAACTGC | ACT |
| rs2715158 | TGTGCAGAAGGTTGACA | ACT |
| rs2715159 | CCTAATAGCCAAAGCAATTTT | ACT |
| rs2522835 | CATATGTCTGTCTGCTTTTA | CGT |
| rs2522836 | CAGTGGTGTGATCTCTG | CGT |
| AE | TTCAAGTTCCTCATAATAACTAC | ACT |
| rs2522837 | CAATGGATTTAACAAATTTGTAG | ACT |
| rs2522838 | ATGCTTGGAGCCATTTC | CGT |
| AF | AATTGTAGATGAAGATGTAGACA | CGT |
| rs2522839 | GACAGAGTGGGACCTGT | ACG |
| rs2522840 | TGCAGAAATGTGACAATG | CGT |
| rs252284 | TTATTACCCATGTGAATTATGATAT | ACT |
| rs10555175 | GACCCTGTCTCAAAAAATAAATAA | CGT |
| rs10592258 | GACCCTGTCTCAAAAAATAAATAA | CGT |
| rs2522842 | GATGCTACATATTCAGCAAAA | CGT |
| rs6956848 | CCTATTAAGCAAAAGACTTTGC | CGT |
| rs2522843 | CAAAGTCTTTTGCTTAATAGG | CGT |
| rs2522844 | ACACCATAAAATGTTAGACATAA | ACT |
| rs2522845 | CTGCTGGCTTCCTACTT | ACT |
| rs2715161 | CTACATATGGAGTGAGAACA | ACG |
| rs6972720 | ATGGGTACCCTTAGACACAC | ACG |
| rs4377905 | AACTGTCTAAGCACTCTAAA | ACT |
| rs4579453 | TCTGCCTCCCTGGTTCA | ACT |
| rs4260846 | ACAAACAAACAAACAAAAAAC | CGT |
| rs1986481 | TTTTGCCAACTATTATCCC | CGT |
| rs4628206 | CATGACACAGACCACAT | CGT |
| rs2888017 | GAAATCCAAATTAAAACCACA | ACT |

Genetic Analysis

Allelotyping results are shown for female (F) and male (M) cases and controls in Table 29 and Table 30, respectively. Allele frequency is noted in the fourth and fifth columns for melanoma pools and control pools, respectively.

TABLE 29
Females

| | | | | | | | | Melanoma |
|---|---|---|---|---|---|---|---|---|
| | Chromo- | Chromosome | | Female | Female | F | Odds | Associated |
| dbSNP rs# | some | Position | Alleles | Case AF | Control AF | p-Value | Ratio | Allele |
| rs2158220 | 7 | 82023020 | T/C | T = | T = | | | |
| | | | | C = | C = | | | |
| rs1024465 | 7 | 82024165 | G/A | G = 0.845 | G = 0.845 | 0.987 | 0.997 | A |
| | | | | A = 0.154 | A = 0.154 | | | |
| rs4732483 | 7 | 82028598 | T/C | T = 0.112 | T = 0.108 | 0.866 | 0.960 | T |
| | | | | C = 0.888 | C = 0.892 | | | |
| rs4732485 | 7 | 82031677 | C/T | C = 0.050 | C = 0.047 | 0.868 | 0.946 | C |
| | | | | T = 0.949 | T = 0.952 | | | |
| rs1859176 | 7 | 82040109 | C/T | C = 0.647 | C = 0.761 | ˜0.001 | 1.738 | T |
| | | | | T = 0.352 | T = 0.238 | | | |
| rs1859177 | 7 | 82040246 | C/T | C = 0.979 | C = 0.981 | 0.655 | 1.291 | T |
| | | | | T = 0.021 | T = 0.019 | | | |
| rs2074401 | 7 | 82041637 | A/G | A = 0.620 | A = 0.557 | 0.096 | 0.771 | A |
| | | | | G = 0.379 | G = 0.442 | | | |
| rs4299948 | 7 | 82041959 | T/G | T = | T = | | | |
| | | | | G = | G = | | | |
| rs4348435 | 7 | 82043702 | G/A | G = 0.735 | G = 0.742 | 0.828 | 1.033 | A |
| | | | | A = 0.264 | A = 0.257 | | | |
| rs4348436 | 7 | 82043723 | G/C | G = 0.813 | G = 0.821 | 0.782 | 1.050 | C |
| | | | | C = 0.186 | C = 0.178 | | | |
| rs4413718 | 7 | 82043866 | G/C | G = 0.960 | G = 0.968 | 0.596 | 1.267 | C |
| | | | | C = 0.039 | C = 0.031 | | | |
| rs985199 | 7 | 82043947 | G/A | G = 0.244 | G = 0.244 | 0.980 | 1.004 | G |
| | | | | A = 0.755 | A = 0.755 | | | |
| rs7791413 | 7 | 82051808 | T/C | T = 0.697 | T = 0.650 | 0.195 | 0.809 | T |
| | | | | C = 0.302 | C = 0.349 | | | |
| rs2522830 | 7 | 82055154 | G/A | G = 0.630 | G = 0.569 | 0.082 | 0.775 | G |
| | | | | A = 0.369 | A = 0.430 | | | |
| rs2214414 | 7 | 82056163 | T/C | T = 0.582 | T = 0.651 | 0.049 | 1.340 | C |
| | | | | C = 0.417 | C = 0.348 | | | |
| rs2189246 | 7 | 82056423 | A/G | A = 0.160 | A = 0.126 | 0.197 | 0.756 | A |
| | | | | G = 0.839 | G = 0.873 | | | |
| rs5885312 | 7 | 82056469 | CTTAAGAA/— | CTTAAGAA = 0.070 | CTTAAGAA = 0.101 | 0.137 | 1.504 | — |
| | | | | — = 0.929 | — = 0.898 | | | |
| rs3064840 | 7 | 82056472 | AAGAACTT/— | AAGAACTT = 0.042 | AAGAACTT = 0.050 | 0.624 | 1.196 | — |
| | | | | — = 0.957 | — = 0.949 | | | |
| rs2051791 | 7 | 82056789 | C/T | C = 0.449 | C = 0.541 | 0.007 | 1.445 | T |
| | | | | T = 0.550 | T = 0.458 | | | |
| rs6467914 | 7 | 82057396 | C/T | C = 0.121 | C = 0.204 | 0.001 | 1.852 | T |
| | | | | T = 0.878 | T = 0.795 | | | |
| rs2214415 | 7 | 82057533 | G/A | G = 0.576 | G = 0.611 | 0.387 | 1.156 | A |
| | | | | A = 0.423 | A = 0.388 | | | |
| rs2189247 | 7 | 82059296 | C/A | C = 0.689 | C = 0.607 | 0.027 | 0.697 | C |
| | | | | A = 0.310 | A = 0.392 | | | |
| rs3064869 | 7 | 82059469 | —/ACA | — = 0.555 | — = 0.635 | 0.015 | 1.393 | A |
| | | | | ACA = 0.444 | ACA = 0.364 | | | |
| rs2522831 | 7 | 82060151 | T/C | T = | T = | | | |
| | | | | C = | C = | | | |
| rs2715147 | 7 | 82060456 | T/C | T = 0.597 | T = 0.656 | 0.090 | 1.285 | C |
| | | | | C = 0.402 | C = 0.343 | | | |
| rs3064870 | 7 | 82060855 | —/TAAG | — = 0.279 | — = 0.252 | 0.398 | 0.872 | — |
| | | | | TAAG = 0.720 | TAAG = 0.747 | | | |
| rs2715148 | 7 | 82062086 | A/C | A = 0.558 | A = 0.463 | 0.004 | 0.681 | A |
| | | | | C = 0.441 | C = 0.536 | | | |
| rs2522832 | 7 | 82062415 | A/G | A = | A = | | | |
| | | | | G = | G = | | | |
| rs1044639 | 7 | 82063609 | G/T | G = 0.413 | G = 0.485 | 0.026 | 1.339 | T |
| | | | | T = 0.586 | T = 0.514 | | | |
| rs2715149 | 7 | 82064863 | A/T | A = 0.927 | A = 0.954 | 0.256 | 1.648 | T |
| | | | | T = 0.072 | T = 0.045 | | | |
| rs2522833 | 7 | 82065759 | C/A | C = 0.384 | C = 0.523 | ˜0.001 | 1.762 | A |
| | | | | A = 0.615 | A = 0.476 | | | |
| rs2247523 | 7 | 82066455 | C/G | C = 0.633 | C = 0.575 | 0.079 | 0.783 | C |
| | | | | G = 0.366 | G = 0.424 | | | |
| rs2371214 | 7 | 82066723 | T/C | T = 0.637 | T = 0.569 | 0.042 | 0.752 | T |
| | | | | C = 0.362 | C = 0.430 | | | |
| rs2189248 | 7 | 82067356 | T/C | T = 0.276 | T = 0.274 | 0.945 | 0.990 | T |
| | | | | C = 0.723 | C = 0.725 | | | |
| rs2715150 | 7 | 82067946 | A/G | A = 0.787 | A = 0.749 | 0.205 | 0.811 | A |
| | | | | G = 0.212 | G = 0.250 | | | |
| rs2715151 | 7 | 82068714 | C/G | C = 0.632 | C = 0.546 | 0.010 | 0.699 | C |
| | | | | G = 0.367 | G = 0.453 | | | |
| rs2371215 | 7 | 82069545 | T/A | T = 0.626 | T = 0.536 | 0.008 | 0.688 | T |
| | | | | A = 0.373 | A = 0.463 | | | |
| rs2715152 | 7 | 82069717 | G/A | G = 0.680 | G = 0.649 | 0.322 | 0.868 | G |
| | | | | A = 0.319 | A = 0.350 | | | |
| rs2715153 | 7 | 82069909 | T/C | T = 0.549 | T = 0.604 | 0.168 | 1.254 | C |
| | | | | C = 0.450 | C = 0.395 | | | |
| rs972346 | 7 | 82072586 | C/T | C = 0.949 | C = 0.957 | 0.631 | 1.182 | T |
| | | | | T = 0.050 | T = 0.042 | | | |
| rs2715154 | 7 | 82072917 | G/T | G = 0.652 | G = 0.558 | 0.006 | 0.673 | G |
| | | | | T = 0.347 | T = 0.441 | | | |
| rs2522834 | 7 | 82072997 | T/C | T = 0.424 | T = 0.424 | 0.995 | 0.999 | C |
| | | | | C = 0.575 | C = 0.575 | | | |
| rs2023847 | 7 | 82073344 | G/T | G = 0.464 | G = 0.357 | 0.001 | 0.641 | G |
| | | | | T = 0.535 | T = 0.642 | | | |
| rs2715155 | 7 | 82074105 | A/G | A = 0.674 | A = 0.592 | 0.026 | 0.701 | A |
| | | | | G = 0.325 | G = 0.407 | | | |
| rs2715156 | 7 | 82074885 | A/G | A = 0.496 | A = 0.556 | 0.080 | 1.276 | G |
| | | | | G = 0.503 | G = 0.443 | | | |
| rs10715043 | 7 | 82076323 | —/G | — = 0.501 | — = 0.558 | 0.082 | 1.258 | G |
| | | | | G = 0.498 | G = 0.441 | | | |
| AD | 7 | 82076643 | C/G | C = 0.973 | C = 0.965 | 0.582 | 0.773 | C |
| | | | | G = 0.026 | G = 0.034 | | | |
| rs2301722 | 7 | 82077285 | A/C | A = 0.966 | A = 0.961 | 0.729 | 0.864 | A |
| | | | | C = 0.033 | C = 0.038 | | | |
| rs2257207 | 7 | 82080235 | A/T | A = 0.612 | A = 0.557 | 0.123 | 0.798 | A |
| | | | | T = 0.387 | T = 0.442 | | | |
| rs2715157 | 7 | 82080425 | A/G | A = 0.614 | A = 0.529 | 0.009 | 0.707 | A |
| | | | | G = 0.385 | G = 0.470 | | | |
| rs2715158 | 7 | 82081657 | G/C | G = 0.742 | G = 0.680 | 0.048 | 0.740 | G |
| | | | | C = 0.257 | C = 0.319 | | | |
| rs2715159 | 7 | 82083459 | A/G | A = 0.726 | A = 0.619 | ˜0.001 | 0.613 | A |
| | | | | G = 0.273 | G = 0.380 | | | |
| rs2522835 | 7 | 82083547 | A/T | A = 0.542 | A = 0.685 | ˜0.001 | 1.840 | T |
| | | | | T = 0.457 | T = 0.314 | | | |
| rs2522836 | 7 | 82085104 | C/A | C = 0.532 | C = 0.519 | 0.714 | 0.948 | C |
| | | | | A = 0.467 | A = 0.480 | | | |
| AE | 7 | 82086424 | T/G | T = 0.957 | T = 0.991 | 0.009 | 5.183 | G |
| | | | | G = 0.043 | G = 0.009 | | | |
| rs2522837 | 7 | 82087433 | A/G | A = 0.649 | A = 0.564 | 0.019 | 0.700 | A |
| | | | | G = 0.350 | G = 0.435 | | | |
| rs2522838 | 7 | 82087608 | A/T | A = 0.458 | A = 0.512 | 0.127 | 1.239 | T |
| | | | | T = 0.541 | T = 0.487 | | | |
| AF | 7 | 82087839 | AT/— | AT = 0.668 | AT = 0.592 | 0.028 | 0.721 | A |
| | | | | — = 0.331 | — = 0.407 | | | |
| rs2522839 | 7 | 82089699 | G/A | G = 0.650 | G = 0.676 | 0.534 | 1.126 | A |
| | | | | A = 0.349 | A = 0.323 | | | |
| rs2522840 | 7 | 82090466 | G/T | G = 0.511 | G = 0.555 | 0.288 | 1.193 | T |
| | | | | T = 0.488 | T = 0.444 | | | |
| rs2522841 | 7 | 82091245 | T/G | T = 0.422 | T = 0.488 | 0.067 | 1.305 | G |
| | | | | G = 0.577 | G = 0.511 | | | |
| rs10555175 | 7 | 82091377 | —/TTAT | — = 0.623 | — = 0.707 | 0.018 | 1.460 | T |
| | | | | TTAT = 0.376 | TTAT = 0.292 | | | |
| rs10592258 | 7 | 82091381 | —/TATTATT | — = 0.618 | — = 0.682 | 0.073 | 1.326 | T |
| | | | | TATTATT = 0.381 | TATTATT = 0.317 | | | |
| rs2522842 | 7 | 82091713 | G/T | G = 0.648 | G = 0.584 | 0.062 | 0.764 | G |
| | | | | T = 0.351 | T = 0.415 | | | |
| rs6956848 | 7 | 82091750 | G/T | G = | G = | | | |
| | | | | T = | T = | | | |
| rs2522843 | 7 | 82091773 | C/A | C = 0.567 | C = 0.569 | 0.956 | 1.007 | A |
| | | | | A = 0.432 | A = 0.430 | | | |
| rs2522844 | 7 | 82092143 | T/C | T = 0.459 | T = 0.517 | 0.109 | 1.262 | C |
| | | | | C = 0.540 | C = 0.482 | | | |
| rs2522845 | 7 | 82093960 | T/G | T = 0.657 | T = 0.592 | 0.045 | 0.757 | T |
| | | | | G = 0.342 | G = 0.407 | | | |
| rs2715161 | 7 | 82094332 | C/T | C = 0.616 | C = 0.558 | 0.083 | 0.786 | C |
| | | | | T = 0.383 | T = 0.441 | | | |
| rs6972720 | 7 | 82094726 | C/T | C = 0.777 | C = 0.779 | 0.940 | 1.012 | T |
| | | | | T = 0.222 | T = 0.220 | | | |
| rs4377905 | 7 | 82106362 | A/G | A = 0.955 | A = 0.960 | 0.760 | 1.123 | G |
| | | | | G = 0.044 | G = 0.039 | | | |
| rs4579453 | 7 | 82106772 | T/C | T = 0.954 | T = 0.911 | 0.016 | 0.490 | T |
| | | | | C = 0.045 | C = 0.088 | | | |
| rs4260846 | 7 | 82106887 | C/A | C = 0.729 | C = 0.709 | 0.510 | 0.906 | C |
| | | | | A = 0.270 | A = 0.290 | | | |
| rs1986481 | 7 | 82108734 | A/T | A = 0.437 | A = 0.375 | 0.058 | 0.772 | A |
| | | | | T = 0.562 | T = 0.624 | | | |
| rs4628206 | 7 | 82109394 | A/T | A = 0.854 | A = 0.846 | 0.757 | 0.941 | A |
| | | | | T = 0.145 | T = 0.153 | | | |
| rs2888017 | 7 | 82112254 | A/G | A = 0.868 | A = 0.865 | 0.907 | 0.976 | A |
| | | | | G = 0.131 | G = 0.134 | | | |

TABLE 30
Males

| | | | | | | | | Melanoma |
|---|---|---|---|---|---|---|---|---|
| | Chromo- | Chromosome | | Male | Male | M | Odds | Associated |
| dbSNP rs# | some | Position | Alleles | Case AF | Control AF | p-Value | Ratio | Allele |
| rs2158220 | 7 | 82023020 | T/C | T = 0.740 | T = 0.682 | 0.076 | 0.753 | T |
| | | | | C = 0.259 | C = 0.317 | | | |
| rs1024465 | 7 | 82024165 | G/A | G = 0.864 | G = 0.878 | 0.622 | 1.127 | A |
| | | | | A = 0.135 | A = 0.121 | | | |
| rs4732483 | 7 | 82028598 | T/C | T = 0.115 | T = 0.111 | 0.903 | 0.970 | T |
| | | | | C = 0.885 | C = 0.889 | | | |
| rs4732485 | 7 | 82031677 | C/T | C = 0.040 | C = 0.030 | 0.572 | 0.741 | C |
| | | | | T = 0.959 | T = 0.969 | | | |
| rs1859176 | 7 | 82040109 | C/T | C = | C = | | | |
| | | | | T = | T = | | | |
| rs1859177 | 7 | 82040246 | C/T | C = 0.989 | C = 0.985 | 0.743 | 0.716 | C |
| | | | | T = 0.011 | T = 0.015 | | | |
| rs2074401 | 7 | 82041637 | A/G | A = 0.633 | A = 0.574 | 0.095 | 0.781 | A |
| | | | | G = 0.366 | G = 0.425 | | | |
| rs4299948 | 7 | 82041959 | T/G | T = 0.089 | T = 0.337 | 1.332 | 5.184 | G |
| | | | | G = 0.910 | G = 0.662 | | | |
| rs4348435 | 7 | 82043702 | G/A | G = 0.760 | G = 0.772 | 0.669 | 1.073 | A |
| | | | | A = 0.239 | A = 0.227 | | | |
| rs4348436 | 7 | 82043723 | G/C | G = 0.831 | G = 0.864 | 0.205 | 1.293 | C |
| | | | | C = 0.168 | C = 0.135 | | | |
| rs4413718 | 7 | 82043866 | G/C | G = 0.959 | G = 0.972 | 0.375 | 1.486 | C |
| | | | | C = 0.040 | C = 0.027 | | | |
| rs985199 | 7 | 82043947 | G/A | G = 0.224 | G = 0.222 | 0.949 | 0.989 | G |
| | | | | A = 0.775 | A = 0.777 | | | |
| rs7791413 | 7 | 82051808 | T/C | T = 0.725 | T = 0.698 | 0.396 | 0.877 | T |
| | | | | C = 0.274 | C = 0.301 | | | |
| rs2522830 | 7 | 82055154 | G/A | G = 0.659 | G = 0.574 | 0.015 | 0.696 | G |
| | | | | A = 0.340 | A = 0.425 | | | |
| rs2214414 | 7 | 82056163 | T/C | T = 0.556 | T = 0.632 | 0.054 | 1.369 | C |
| | | | | C = 0.443 | C = 0.367 | | | |
| rs2189246 | 7 | 82056423 | A/G | A = | A = | | | |
| | | | | G = | G = | | | |
| rs5885312 | 7 | 82056469 | CTTAAGAA/— | CTTAAGAA = 0.057 | CTTAAGAA = 0.061 | 0.803 | 1.083 | — |
| | | | | — = 0.942 | — = 0.938 | | | |
| rs3064840 | 7 | 82056472 | AAGAACTT/— | AAGAACTT = 0.043 | AAGAACTT = 0.039 | 0.859 | 0.919 | A |
| | | | | — = 0.956 | — = 0.960 | | | |
| rs2051791 | 7 | 82056789 | C/T | C = 0.469 | C = 0.562 | 0.025 | 1.455 | T |
| | | | | T = 0.530 | T = 0.437 | | | |
| rs6467914 | 7 | 82057396 | C/T | C = 0.149 | C = 0.164 | 0.569 | 1.121 | T |
| | | | | T = 0.850 | T = 0.835 | | | |
| rs2214415 | 7 | 82057533 | G/A | G = 0.623 | G = 0.621 | 0.976 | 0.994 | G |
| | | | | A = 0.376 | A = 0.378 | | | |
| rs2189247 | 7 | 82059296 | C/A | C = 0.653 | C = 0.626 | 0.408 | 0.886 | C |
| | | | | A = 0.346 | A = 0.373 | | | |
| rs3064869 | 7 | 82059469 | —/ACA | — = 0.553 | — = 0.605 | 0.127 | 1.238 | A |
| | | | | ACA = 0.446 | ACA = 0.394 | | | |
| rs2522831 | 7 | 82060151 | T/C | T = 0.714 | T = 0.699 | 0.709 | 0.932 | T |
| | | | | C = 0.285 | C = 0.300 | | | |
| rs2715147 | 7 | 82060456 | T/C | T = 0.572 | T = 0.611 | 0.338 | 1.176 | C |
| | | | | C = 0.427 | C = 0.388 | | | |
| rs3064870 | 7 | 82060855 | —/TAAG | — = 0.231 | — = 0.207 | 0.420 | 0.869 | — |
| | | | | TAAG = 0.768 | TAAG = 0.792 | | | |
| rs2715148 | 7 | 82062086 | A/C | A = 0.557 | A = 0.502 | 0.141 | 0.802 | A |
| | | | | C = 0.442 | C = 0.497 | | | |
| rs2522832 | 7 | 82062415 | A/G | A = 0.662 | A = 0.628 | 0.335 | 0.861 | A |
| | | | | G = 0.337 | G = 0.371 | | | |
| rs1044639 | 7 | 82063609 | G/T | G = 0.429 | G = 0.488 | 0.090 | 1.273 | T |
| | | | | T = 0.570 | T = 0.511 | | | |
| rs2715149 | 7 | 82064863 | A/T | A = 0.907 | A = 0.940 | 0.097 | 1.620 | T |
| | | | | T = 0.092 | T = 0.059 | | | |
| rs2522833 | 7 | 82065759 | C/A | C = 0.396 | C = 0.503 | 0.003 | 1.543 | A |
| | | | | A = 0.603 | A = 0.496 | | | |
| rs2247523 | 7 | 82066455 | C/G | C = 0.646 | C = 0.582 | 0.061 | 0.764 | C |
| | | | | G = 0.353 | G = 0.417 | | | |
| rs2371214 | 7 | 82066723 | T/C | T = 0.619 | T = 0.592 | 0.471 | 0.893 | T |
| | | | | C = 0.380 | C = 0.407 | | | |
| rs2189248 | 7 | 82067356 | T/C | T = 0.246 | T = 0.238 | 0.821 | 0.958 | T |
| | | | | C = 0.753 | C = 0.761 | | | |
| rs2715150 | 7 | 82067946 | A/G | A = 0.802 | A = 0.778 | 0.441 | 0.868 | A |
| | | | | G = 0.197 | G = 0.221 | | | |
| rs2715151 | 7 | 82068714 | C/G | C = 0.631 | C = 0.597 | 0.414 | 0.864 | C |
| | | | | G = 0.368 | G = 0.402 | | | |
| rs2371215 | 7 | 82069545 | T/A | T = 0.620 | T = 0.544 | 0.025 | 0.731 | T |
| | | | | A = 0.379 | A = 0.455 | | | |
| rs2715152 | 7 | 82069717 | G/A | G = 0.718 | G = 0.688 | 0.450 | 0.866 | G |
| | | | | A = 0.281 | A = 0.311 | | | |
| rs2715153 | 7 | 82069909 | T/C | T = 0.491 | T = 0.595 | 0.016 | 1.523 | C |
| | | | | C = 0.508 | C = 0.404 | | | |
| rs972346 | 7 | 82072586 | C/T | C = 0.959 | C = 0.970 | 0.485 | 1.362 | T |
| | | | | T = 0.040 | T = 0.029 | | | |
| rs2715154 | 7 | 82072917 | G/T | G = 0.632 | G = 0.573 | 0.084 | 0.784 | G |
| | | | | T = 0.367 | T = 0.426 | | | |
| rs2522834 | 7 | 82072997 | T/C | T = 0.386 | T = 0.428 | 0.222 | 1.189 | C |
| | | | | C = 0.613 | C = 0.571 | | | |
| rs2023847 | 7 | 82073344 | G/T | G = 0.467 | G = 0.400 | 0.052 | 0.761 | G |
| | | | | T = 0.532 | T = 0.599 | | | |
| rs2715155 | 7 | 82074105 | A/G | A = | A = | | | |
| | | | | G = | G = | | | |
| rs2715156 | 7 | 82074885 | A/G | A = 0.411 | A = 0.533 | 0.006 | 1.634 | G |
| | | | | G = 0.588 | G = 0.466 | | | |
| rs10715043 | 7 | 82076323 | —/G | — = 0.474 | — = 0.533 | 0.092 | 1.263 | G |
| | | | | G = 0.525 | G = 0.466 | | | |
| AD | 7 | 82076643 | C/G | C = 0.967 | C = 0.977 | 0.506 | 1.410 | G |
| | | | | G = 0.032 | G = 0.022 | | | |
| rs2301722 | 7 | 82077285 | A/C | A = 0.965 | A = 0.976 | 0.418 | 1.487 | C |
| | | | | C = 0.034 | C = 0.023 | | | |
| rs2257207 | 7 | 82080235 | A/T | A = 0.622 | A = 0.582 | 0.234 | 0.843 | A |
| | | | | T = 0.377 | T = 0.417 | | | |
| rs2715157 | 7 | 82080425 | A/G | A = | A = | | | |
| | | | | G = | G = | | | |
| rs2715158 | 7 | 82081657 | G/C | G = 0.748 | G = 0.682 | 0.091 | 0.724 | G |
| | | | | C = 0.251 | C = 0.317 | | | |
| rs2715159 | 7 | 82083459 | A/G | A = 0.733 | A = 0.634 | 0.002 | 0.632 | A |
| | | | | G = 0.266 | G = 0.365 | | | |
| rs2522835 | 7 | 82083547 | A/T | A = 0.534 | A = 0.651 | ˜0.001 | 1.630 | T |
| | | | | T = 0.465 | T = 0.348 | | | |
| rs2522836 | 7 | 82085104 | C/A | C = 0.570 | C = 0.544 | 0.506 | 0.899 | C |
| | | | | A = 0.429 | A = 0.455 | | | |
| AE | 7 | 82086424 | T/G | T = 0.940 | T = 0.967 | 0.217 | 1.886 | G |
| | | | | G = 0.059 | G = 0.032 | | | |
| rs2522837 | 7 | 82087433 | A/G | A = 0.639 | A = 0.606 | 0.341 | 0.869 | A |
| | | | | G = 0.360 | G = 0.393 | | | |
| rs2522838 | 7 | 82087608 | A/T | A = 0.451 | A = 0.517 | 0.078 | 1.301 | T |
| | | | | T = 0.548 | T = 0.482 | | | |
| AF | 7 | 82087839 | AT/— | AT = 0.676 | AT = 0.592 | 0.028 | 0.721 | A |
| | | | | — = 0.323 | — = 0.407 | | | |
| rs2522839 | 7 | 82089699 | G/A | G = | G = | | | |
| | | | | A = | A = | | | |
| rs2522840 | 7 | 82090466 | G/T | G = 0.488 | G = 0.550 | 0.101 | 1.284 | T |
| | | | | T = 0.511 | T = 0.449 | | | |
| rs2522841 | 7 | 82091245 | T/G | T = 0.411 | T = 0.462 | 0.181 | 1.230 | G |
| | | | | G = 0.588 | G = 0.537 | | | |
| rs10555175 | 7 | 82091377 | —/TTAT | — = 0.620 | — = 0.679 | 0.089 | 1.294 | T |
| | | | | TTAT = 0.379 | TTAT = 0.320 | | | |
| rs10592258 | 7 | 82091381 | —/TATTATT | — = 0.613 | — = 0.670 | 0.090 | 1.283 | T |
| | | | | TATTATT = 0.386 | TATTATT = 0.329 | | | |
| rs2522842 | 7 | 82091713 | G/T | G = 0.647 | G = 0.603 | 0.190 | 0.829 | G |
| | | | | T = 0.352 | T = 0.396 | | | |
| rs6956848 | 7 | 82091750 | G/T | G = 0.035 | G = 0.150 | ˜0.001 | 4.818 | T |
| | | | | T = 0.964 | T = 0.849 | | | |
| rs2522843 | 7 | 82091773 | C/A | C = 0.576 | C = 0.542 | 0.352 | 0.872 | C |
| | | | | A = 0.423 | A = 0.457 | | | |
| rs2522844 | 7 | 82092143 | T/C | T = 0.458 | T = 0.501 | 0.219 | 1.187 | C |
| | | | | C = 0.541 | C = 0.498 | | | |
| rs2522845 | 7 | 82093960 | T/G | T = 0.677 | T = 0.604 | 0.047 | 0.725 | T |
| | | | | G = 0.322 | G = 0.395 | | | |
| rs2715161 | 7 | 82094332 | C/T | C = 0.666 | C = 0.591 | 0.051 | 0.725 | C |
| | | | | T = 0.333 | T = 0.408 | | | |
| rs6972720 | 7 | 82094726 | C/T | C = 0.796 | C = 0.791 | 0.839 | 0.966 | C |
| | | | | T = 0.203 | T = 0.208 | | | |
| rs4377905 | 7 | 82106362 | A/G | A = 0.955 | A = 0.975 | 0.180 | 1.828 | G |
| | | | | G = 0.044 | G = 0.024 | | | |
| rs4579453 | 7 | 82106772 | T/C | T = 0.959 | T = 0.899 | 0.006 | 0.382 | T |
| | | | | C = 0.040 | C = 0.100 | | | |
| rs4260846 | 7 | 82106887 | C/A | C = 0.726 | C = 0.707 | 0.590 | 0.912 | C |
| | | | | A = 0.273 | A = 0.292 | | | |
| rs1986481 | 7 | 82108734 | A/T | A = 0.485 | A = 0.417 | 0.049 | 0.760 | A |
| | | | | T = 0.514 | T = 0.582 | | | |
| rs4628206 | 7 | 82109394 | A/T | A = 0.863 | A = 0.858 | 0.826 | 0.954 | A |
| | | | | T = 0.136 | T = 0.141 | | | |
| rs2888017 | 7 | 82112254 | A/G | A = 0.902 | A = 0.895 | 0.767 | 0.927 | A |
| | | | | G = 0.097 | G = 0.104 | | | |

Allelotyping results were considered significant with a calculated p-value of less than or equal to 0.05 for allelotype results. These values are indicated in bold. The assay failed for those SNPs in which the allele frequency is blank. The combined allelotyping p-values for males and females were plotted in FIG. 4A and separately for females and males in FIGS. 4B and 4C, respectively. The position of each SNP on the chromosome is presented on the x-axis. The y-axis gives the negative logarithm (base 10) of the p-value comparing the estimated allele frequency in the case group to that of the control group. The minor allele frequency of the control group for each SNP designated by an X or other symbol on the graphs in FIGS. 4A, 4B and 4C can be determined by consulting Table 29 or 30. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brooks/Cole.). The black line (or generally top-most curve, e.g., see peak in left-most graph just to the left of position 92150000) provides a local test for excess statistical significance to identify regions of association. This was created by use of a 10 kb sliding window with 1 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 10⁻⁸ were truncated at that value.

Finally, the exons and introns of the genes in the covered region are plotted below each graph at the appropriate chromosomal positions. The gene boundary is indicated by the broken horizontal line. The exon positions are shown as thick, unbroken bars. An arrow is place at the 3′ end of each gene to show the direction of transcription.

### Example 9

### FPGT/CARK Proximal SNPs

It has been discovered that a polymorphic variation (rs2034453) in an intron of a gene encoding cardiac troponin I interacting kinase (TNNI3K), also referred to as cardiac ankyrin repeat kinase (CARK), and near a gene encoding fucose-1-phosphate guanylyltransferase (FPGT) is associated with the occurrence of melanoma. See Table 3. Subsequently, ninety-two allelic variants located within or nearby the target genes were identified and subsequently allelotyped in melanoma case and control sample sets as described in Examples 1 and 2. Several SNPs, designated by SNP_ID “AG”, “AH”, and “AI” in column 1 of Table 20, are novel and were identified through SNP discovery methods using dye terminator sequencing. Methods of diedeoxynucleotide chain termination chemistry derived sequencing are described by Sanger et al. (Proc. Nat. Acad. Sci. USA 74, 5463-5467, 1997). Briefly, terminators are labeled with fluorescent dyes for automated detection. Fluorescein is measured by an argon ion laser in the sequencing instrument, which transfers light energy to a rhodamine acceptor dye. Each acceptor dye emits light at a characteristic wavelength for detection, identifying the nucleotide that terminated extension of the DNA fragment. Novel SNPs are identified when sequence from multiple samples are compared on one or more strands. The forward and reverse primers used to identify AG are GGGTGGCTCACTCTCTAGGAA and CCTAGCAGCTGCCATAGCC, respectively. The forward and reverse primers used to identify AH are AACTTGTCCACAACCCTCAGTT and TGAAGAGTTTGAATGTGAAGCTG, respectively. The forward and reverse primers used to identify AI are TGGCTTAGGAGAAAATGATCTTG and TTTTCTGTAACATTATACCAAGGAAC, respectively. The polymorphic variants are set forth in Table 31. The chromosome position provided in column four of Table 31 is based on Genome “Build 34” of NCBI's GenBank and dbSNP build “119”.

TABLE 31

| | | Position | | | Allele | |
|---|---|---|---|---|---|---|
| | Chromo- | in SEQ | Chromosome | Allele | Present in | IUPAC |
| dbSNP rs# | some | ID NO: 5 | Position | Variants | SEQ ID NO: 5 | Code |
| AG | 1 | 206 | 74033656 | G/T | T | K |
| rs1412825 | 1 | 299 | 74033749 | G/C | C | S |
| rs4422957 | 1 | 634 | 74034084 | C/T | T | Y |
| rs3835390 | 1 | 990 | 74034440 | AAAAG/— | A | N |
| rs944795 | 1 | 1,433 | 74034883 | C/G | G | S |
| rs792310 | 1 | 1,978 | 74035428 | G/A | G | R |
| rs526736 | 1 | 2,227 | 74035677 | A/T | A | W |
| rs9804006 | 1 | 2,992 | 74036442 | T/C | T | Y |
| rs577367 | 1 | 4,171 | 74037621 | C/T | A | R |
| rs575754 | 1 | 4,313 | 74037763 | T/C | G | R |
| rs573721 | 1 | 4,586 | 74038036 | G/T | C | M |
| rs487917 | 1 | 5,381 | 74038831 | C/T | G | R |
| rs792307 | 1 | 5,485 | 74038935 | G/A | C | Y |
| rs476350 | 1 | 5,792 | 74039242 | T/C | C | Y |
| rs9425023 | 1 | 6,396 | 74039846 | T/C | T | Y |
| rs9425024 | 1 | 6,458 | 74039908 | G/T | T | K |
| rs571848 | 1 | 8,004 | 74041454 | A/G | A | R |
| rs489941 | 1 | 8,808 | 74042258 | A/C | C | M |
| rs491623 | 1 | 8,959 | 74042409 | A/G | G | R |
| rs956 | 1 | 9,125 | 74042575 | T/A | T | W |
| AH | 1 | 9,261 | 74042711 | G/A | G | R |
| rs520806 | 1 | 9,826 | 74043276 | C/T | C | Y |
| rs522759 | 1 | 10,067 | 74043517 | T/C | T | Y |
| rs545664 | 1 | 10,262 | 74043712 | G/A | A | R |
| rs545721 | 1 | 10,284 | 74043734 | C/T | T | Y |
| rs536355 | 1 | 10,661 | 74044111 | G/A | A | R |
| rs553044 | 1 | 11,058 | 74044508 | G/T | T | K |
| rs581353 | 1 | 11,871 | 74045321 | A/G | A | R |
| rs496008 | 1 | 12,291 | 74045741 | T/A | A | W |
| rs7366854 | 1 | 13,150 | 74046600 | C/T | C | Y |
| rs560808 | 1 | 14,718 | 74048168 | C/A | C | M |
| rs485929 | 1 | 14,770 | 74048220 | A/G | A | R |
| rs477134 | 1 | 15,399 | 74048849 | T/G | T | K |
| rs505634 | 1 | 16,136 | 74049586 | G/A | A | R |
| rs542136 | 1 | 16,345 | 74049795 | G/A | A | R |
| rs545680 | 1 | 16,725 | 74050175 | T/C | T | Y |
| rs522042 | 1 | 17,567 | 74051017 | G/A | G | R |
| rs575961 | 1 | 17,695 | 74051145 | C/T | T | Y |
| rs792321 | 1 | 18,453 | 74051903 | A/G | A | R |
| rs524306 | 1 | 18,738 | 74052188 | T/A | A | W |
| rs495027 | 1 | 18,744 | 74052194 | A/C | C | M |
| rs525358 | 1 | 19,789 | 74053239 | A/G | A | R |
| rs471496 | 1 | 20,391 | 74053841 | G/A | G | R |
| rs504866 | 1 | 21,725 | 74055175 | C/T | T | Y |
| rs532396 | 1 | 23,377 | 74056827 | T/C | C | Y |
| rs533371 | 1 | 23,497 | 74056947 | G/A | A | R |
| rs567060 | 1 | 24,849 | 74058299 | A/G | A | R |
| rs4650251 | 1 | 30,033 | 74063483 | A/G | G | R |
| rs3753183 | 1 | 35,584 | 74069034 | G/T | T | K |
| rs518769 | 1 | 36,397 | 74069847 | A/G | G | R |
| rs474215 | 1 | 36,736 | 74070186 | T/C | C | Y |
| AI | 1 | 37,592 | 74071042 | T/C | C | Y |
| rs500203 | 1 | 38,136 | 74071586 | T/A | A | W |
| rs532221 | 1 | 38,435 | 74071885 | T/A | T | W |
| rs539534 | 1 | 39,222 | 74072672 | C/T | T | Y |
| rs531412 | 1 | 39,270 | 74072720 | G/A | A | R |
| rs9970514 | 1 | 40,243 | 74073693 | C/G | G | S |
| rs568049 | 1 | 40,988 | 74074438 | C/T | T | Y |
| rs9727250 | 1 | 44,065 | 74077515 | T/C | C | Y |
| rs792323 | 1 | 44,079 | 74077529 | C/T | C | Y |
| rs3765651 | 1 | 45,847 | 74079297 | A/C | C | M |
| rs3765654 | 1 | 46,196 | 74079646 | C/G | C | S |
| rs481387 | 1 | 46,935 | 74080385 | C/T | C | Y |
| rs483259 | 1 | 47,114 | 74080564 | T/C | C | Y |
| rs2034453 | 1 | 48,666 | 74082116 | A/G | A | R |
| rs7536997 | 1 | 49,437 | 74082887 | G/A | G | R |
| rs792324 | 1 | 49,556 | 74083006 | T/C | T | Y |
| rs6660350 | 1 | 50,036 | 74083486 | C/T | C | Y |
| rs492302 | 1 | 50,624 | 74084074 | G/C | T | S |
| rs10493539 | 1 | 52,062 | 74085512 | A/G | A | R |
| rs496720 | 1 | 55,198 | 74088648 | G/A | A | R |
| rs570631 | 1 | 56,340 | 74089790 | T/A | T | W |
| rs4593767 | 1 | 59,578 | 74093028 | G/A | G | R |
| rs576802 | 1 | 60,781 | 74094231 | T/A | A | W |
| rs518574 | 1 | 67,523 | 74100973 | A/G | A | R |
| rs524252 | 1 | 69,082 | 74102532 | C/T | C | Y |
| rs792327 | 1 | 70,006 | 74103456 | G/A | A | R |
| rs792328 | 1 | 70,437 | 74103887 | C/A | A | M |
| rs2039407 | 1 | 70,855 | 74104305 | A/T | A | W |
| rs503770 | 1 | 71,931 | 74105381 | T/C | T | Y |
| rs503904 | 1 | 71,990 | 74105440 | A/G | A | R |
| rs514012 | 1 | 73,566 | 74107016 | A/G | A | R |
| rs1412827 | 1 | 74,082 | 74107532 | A/G | A | R |
| rs473834 | 1 | 79,519 | 74112969 | G/A | A | R |
| rs480267 | 1 | 80,209 | 74113659 | A/G | G | R |
| rs572180 | 1 | 85,684 | 74119134 | G/T | G | K |
| rs1333029 | 1 | 85,807 | 74119257 | G/A | G | R |
| rs485414 | 1 | 85,927 | 74119377 | A/G | A | R |
| rs1412823 | 1 | 86,251 | 74119701 | A/T | T | W |
| rs2027013 | 1 | 87,438 | 74120888 | T/G | A | M |
| rs548881 | 1 | 95,030 | 74128480 | C/T | C | Y |
| rs792329 | 1 | 97,175 | 74130625 | C/T | A | R |

Assay for Verifying and Allelotyping SNPs

The methods used to verify and allelotype the ninety-two proximal SNPs of Table 31 are the same methods described in Examples 1 and 2 herein. The primers and probes used in these assays are provided in Table 32 and Table 33, respectively.

TABLE 32

| | | |
|---|---|---|
| dbSNP rs # | Forward PCR primer | Reverse PCR primer |

| | | |
|---|---|---|
| AG | ATTTGTCTGACTGTGTTGGG | TTTGTCACCCAAACTGAACC |
| rs1412825 | TTGGCTGGCGCCCGGAAAA | TCCTGAGACCGTTGCTAGAG |
| rs4422957 | GTTTGCTGGACTGTCACTTC | ATTAGCTAGCTACTCCTGGG |
| rs3835390 | TGAGGGAGTAACAGAACATG | AGGGATTAAGTGTCATAGCC |
| rs944795 | GAAGGGATCTTTGGGTTGTG | CCAGCTTTGCAAATCAATTC |
| rs792310 | ACTGACCATAAAGCGCGTAC | AAAGGAGTTACCCCTTGGAG |
| rs526736 | CCTTGAAAAACTTCCAGAATG | ACTGATAAGGTCCAAAGGAG |
| rs9804006 | CACTAGTTATCTTGGGCCAG | AAAGTGCTGGGATTACAGGC |
| rs577367 | TTTTTAAGCATTGTGGCCTC | TACTAGACTTTTGCCAGTAC |
| rs575754 | GAAAAGTAGGAAGGACATGG | CAAAGGTTTTCATGTAACCA |
| rs573721 | CTGGTTTGAAGCCTAAAAATG | CCTGGTATCTAATTTAATTTT |
| rs487917 | TCACCTAGTGATGTTGGAGC | TACCAGCATCACCACTTGAG |
| rs792307 | GGGACAACCTATGTATATGC | AGAGCCAGACTCCATCTCAA |
| rs476350 | ATACCAACACTTTGGGAGGC | GGTTTCACCGTGTTAGCCAG |
| rs9425023 | TATATGCCACTTGTTACTGC | TGGCAACAAGAAAATTTCTC |
| rs9425024 | CCCAGGGTTTTATAACCCAT | GATTTGCAGTAACAAGTGGC |
| rs571848 | CAGGGAACAAATTTTTCTAG | CTCAAAAAGGCAAAGTGTAC |
| rs489941 | TTGTTTCTCAGCATCACCAG | CTCCTATCAGTCCTTCTAAC |
| rs491623 | AGAAGCAGGCAGGAAAATGG | CATAACTTGAGATTCTAGCC |
| rs956 | AGTCTGTGCTACTTTATGTC | CTCCTTGGGAAAAACTTTGG |
| AH | TGATGGCACTTCAGATTTGG | CCAAAGTTTTTCCCAAGGAG |
| rs520806 | GTCAGAAAGAGATTTCTCACA | TTCAGACAGGTCACTTCTGG |
| rs522759 | CAAAGCAACCAGAATCCATG | GCTCAATACTGTATTTTAACC |
| rs545664 | CCCAGAAAACAGAATGTGCC | GACCAAGTTCACATGTGAGC |
| rs545721 | GACCAAGTTCACATGTGAGC | CCCAGAAAACAGAATGTGCC |
| rs536355 | ATAGCAGGCTTTTACACACC | GACCTACATGGATAAGTTTG |
| rs553044 | CAAGTCCAGACGGCATCATG | CTGCCTTCTGTCACATATAG |
| rs581353 | ATCACCTACCTGCACATCAG | CAATCAGTTACTTGCATGCAC |
| rs496008 | ACCAACACATCATAGCTAAG | GAGAAAACAGTGGATACTGG |
| rs7366854 | GGATATTAGCCCTTCGTCAG | CTACCATCAGAGTGAACAGG |
| rs560808 | CAATGCCTTATTGTGTGCAG | AGGCTTTCTGCAACTTTGCG |
| rs485929 | ACGCAAAGTTGCAGAAAGCC | TTCAATGCCTCATACACCCC |
| rs477134 | CTGAATCATTGGAGCTAGGG | CTGTGCTCTGATATGTATCC |
| rs505634 | AGGAGTGAATGTGGATAGAG | TTCTCCTCATCTTCCTCACC |
| rs542136 | CTCTGTTATTGGAAAAAATTC | AAGCCAGCAGGATTATGAAG |
| rs545680 | AATGAAAGTACACTCTACAG | CCCCCACAAATTCTGTAACG |
| rs522042 | GAAGGAGTCTTGCTCTGTTG | AGGAGAATGGCCTGAACCTG |
| rs575961 | GCACAAAATATACCATCGTC | GCACTGGACACCTGTTATTC |
| rs792321 | GTTTCCCATGGCCTAAGATG | CTTCTCTGGTCTCCTGATAG |
| rs524306 | TTGGTTTGGGATAAAGGAGG | AAGCCTCCCAAAAATATGCC |
| rs495027 | CCTAATAATCTAAAGCCTCCC | TTGGTTTGGGATAAAGGAGG |
| rs525358 | ATCTCTTAGTTTGGTACGCG | CACACTACACTTTGACTGGG |
| rs471496 | TCATTTCCAAGAAATATGTG | GTTTATTTACTTTGAACCTG |
| rs504866 | TCTAAATTGTGTCCAGACTC | TAAAAATCTTAGACAGTGGG |
| rs532396 | TTCTAACTGTGGTGTTTCTG | CAAGACTTGTAATCAAATCC |
| rs533371 | GGAAATTGTACATGCAAAGG | TTACTGGTACGGGAAGAATG |
| rs567060 | ATAAACCAAGAGGCATGGTG | AACAAGAGCGAAACCCTGTC |
| rs4650251 | TAGAGCAATCAGAGAAGAGG | GTGGAGTCTTTAGGTTTTTCC |
| rs3753183 | AGCTTTGGCTTTCAAAGGTG | TGTGGGAGAATTCAACTGAG |
| rs518769 | GGAAGCCTTACCTCTGAAAC | CAAACAACTCTCTTTTTGAA |
| rs474215 | CAGTTCTGACCATGTGACTC | ATGGAAGGTGCATGAAAGAG |
| AI | CCAGTACAAGTTTGGGTTGG | GTCACTGCACTTGAACTTGG |
| rs500203 | TCGAATGGGTTCTTCATTAC | GCAAAATGTAGACACAGATC |
| rs532221 | CTTTGTATAAGTGTCTTCAG | TATGTCTGAGGTTCTACGGG |
| rs539534 | GCAATGACCAAATGACACATC | CATGGGTGATTTTATTTGCC |
| rs531412 | CCTTGTTACATCCATTGACG | GCAATGACCAAATGACACATC |
| rs9970514 | TCCTACCATACCCTGCTTAC | TCTTGCCTACACACCTTTAC |
| rs568049 | GATAAGGAAGCTGAGGAGAG | CCATTAACATAAGAGGCCCC |
| rs9727250 | AATGGCGTGAATCCAGGAGG | TTTCAGACGGAGTCTCGCTC |
| rs792323 | TTTTTCAGACGGAGTCTCGC | AATGGCGTGAATCCAGGAGG |
| rs3765651 | CCCAAAGGTTACTTTCTTTC | TGGATCACAAAACTGCACAC |
| rs3765654 | GGCAAAAGAAGAGAAAATGG | CTTCTAGTCAAACGTTTCTG |
| rs481387 | AACCTGAAGTGAGCCTTAAG | CCATTCAATAAGTCTGGCAC |
| rs483259 | CAGAACTGCACATGAGATTC | TTTGAGTCTCTCCACTGTTG |
| rs2034453 | GTGACTGGAAACTGAGAATG | TTGCTGGACAATAGAAAGAC |
| rs7536997 | AGGAATGAACAACTCCGGAC | CGACCTTCGCAGTGAGTGTT |
| rs792324 | TGCTTCTCACTCTTTGGGTC | TGTGATCTTGCTGGCTTCAG |
| rs6660350 | CTAGTAGATCCCACGCAGGG | AGTCCCATCCACCACCCAA |
| rs492302 | TCTTATTTCTAATATCTCGG | TTCCTCCATATGTGGAAAAC |
| rs10493539 | GAAGCACTTAAAGATTCCCC | GGCATTTATCACTTTTCCAC |
| rs496720 | TGTGTCTAGCTCAGGGATTG | TGGTCCGTTTTACAGAGAGC |
| rs570631 | TTGCATTTTGTCCCATTTCC | AAGACTGAAGTGGCAACAGG |
| rs4593767 | ATTGATATATGAAGAAACAG | GTCCAATCTGCTGTTTTCAAC |
| rs576802 | TCTACTTTTGCATTGCTTCC | GGCAATGGATGACAATAGTA |
| rs518574 | GAGCTCCAGAGGAAAAATTG | AGGTGGTGGTAAAAGAAGGG |
| rs524252 | TGGGAGATCTATTCTACCTC | TGGAAGAATGAGGCTTAGAC |
| rs792327 | TGGTCCGTTTTACAGAGAGC | TGTGTCTAGCTCAGGGATTG |
| rs792328 | TCAGCAGGCCATAGCTGCCTC | ATGCAAGAGCCCATGGCAGG |
| rs2039407 | AATGACTATGTGCACTGGAG | TAAGGTGGTTACAATTACTC |
| rs503770 | AATGCATCTTTGGTCCTGAC | TTGAGATGCCATTGATAGGC |
| rs503904 | GAAGTCAGGACCAAAGATGC | CTAAGCTGCTTTCCCATTCC |
| rs514012 | TCTTGTTCAAGCGGTAGAGG | CTCAGATGGTGGAAAGTAGG |
| rs1412827 | GCTTTGGATAGTCAATTCAG | CCTCTGTATATCTAGCTACC |
| rs473834 | TCTTGTGGGTATAAGCTGTG | CTACCTTCACATCTGTAGTC |
| rs480267 | CATCCAAATGTTTCAGTAGAG | TTGGAGAGGAAACTAAAGCG |
| rs572180 | AGCTTTTCTTGCTTATGCTC | CAGGTCTTTGGCATAGACTC |
| rs1333029 | GGGATTTCTCATATCAGGTTG | GCCCTGGAAAATGATGGAAG |
| rs485414 | GCACTGAATTTGCTTCCAAC | GTGTATATACATTATCTACAT |
| rs1412823 | GTATTCCTAATTAAGGGGAAG | AAACCTCTGAGCTCACTATG |
| rs2027013 | CTTCCCGTTTTTCTACCTGC | GAGCCAATGAAAGAAGAAAGG |
| rs548881 | GTAGTTCTATAAGGTTTTGCC | CACCCTGGGAAACTATAAAC |
| rs792329 | CACCACGCCCAGCTAATTTT | GTCAGGAGATCAAGACCATC |

TABLE 33

Term

| | | |
|---|---|---|
| dbSNP rs # | Extend Primer | Mix |

| | | |
|---|---|---|
| AG | CTGTGTTGGGGCTGGGCC | CGT |
| rs1412825 | GCGCCCGGAAAACCATGAT | ACT |
| rs4422957 | CCGTTTACTTCTCATCTG | ACG |
| rs3835390 | TGGAGGAATTATGGATTAAAAATT | ACT |
| rs944795 | CCTAACACCAAGTGTCC | ACT |
| rs792310 | TTGGCTCCAGCAGGATC | ACG |
| rs526736 | TTCCAGAATGTTACTTAAAAGA | CGT |
| rs9804006 | GGGCCAGTTACTTAACTCTTG | ACT |
| rs577367 | GCATTGTGGCCTCTGGCTA | ACG |
| rs575754 | GGACATGGTCTCAGAAT | ACT |
| rs573721 | GAAGCCTAAAAATGTGTAAAA | CGT |
| rs487917 | CCATTGTAAAGTCCTAGCA | ACG |
| rs792307 | GTATATGCTGTCCATAATTGAG | ACG |
| rs476350 | GGCAGGCAGATCACGAG | ACT |
| rs9425023 | TCAGGAAAGAATAATGAAATCCA | ACT |
| rs9425024 | CCCATAAGTTACAAATCAAAATAT | CGT |
| rs571848 | AAAAGCAGTTTGATGTAGAG | ACT |
| rs489941 | TAAAGTCAGTTCATCTGGTAATT | ACT |
| rs491623 | ACAGGTGATAAACAGTTTC | ACT |
| rs956 | GTCAATATAAAAATTGGTAAAC | CGT |
| AH | GAATGACAAAGACATAACATCCT | ACG |
| rs520806 | AAGAGATTTCTCACAAATTGA | ACG |
| rs522759 | CAGAATCCATGCATTGT | ACT |
| rs545664 | GCTATTAGCCAGGCAGCAA | ACG |
| rs545721 | GCTGCCTGGCTAATAGCTA | ACG |
| rs536355 | CTGAGTATATGATGTAAG | ACG |
| rs553044 | GTATGTAAACAGCAACTCTG | CGT |
| rs581353 | GTCTAAAGATGTTCATCCC | ACT |
| rs496008 | TGCACATGTAAATGATTTTTTT | CGT |
| rs7366854 | CTTCGTCAGATGAGTAGGTTG | ACG |
| rs560808 | GTGTGCAGTAATAACAATGAT | CGT |
| rs485929 | AGAAAGCCTGCAAAGGA | ACT |
| rs477134 | TGGAGCTAGGGAGGAAG | ACT |
| rs505634 | ATGTGGATAGAGAAATTCAAA | ACG |
| rs542136 | TTTTCTTCCTTGTAGCATC | ACG |
| rs545680 | CACTCTACAGTGTGGGA | ACT |
| rs522042 | TAGCTCACTCCAAGCTC | ACG |
| rs575961 | CATCGTCATAGTAGAGATTC | ACG |
| rs792321 | AAGATGAATGGACTCTCCTGT | ACT |
| rs524306 | GGACATATTTACTTCTAGCTGAT | CGT |
| rs495027 | CCTCCCAAAAATATGCCTTTG | ACT |
| rs525358 | CGAGAGTGCTCGGTGCATA | ACT |
| rs471496 | GAAATATGTGTTTTAACGAGAA | ACG |
| rs504866 | GTGTCCAGACTCTTGATT | ACG |
| rs532396 | CTGTGGTGTTTCTGATGCCT | ACT |
| rs533371 | TGCAAAGGTGTTAGAAGCACCTC | ACG |
| rs567060 | AGGCATGGTGCTTAGAT | ACT |
| rs4650251 | GAAGAGGGAAAAAAGGACTTC | ACT |
| rs3753183 | CGATGAGTTTGAAAATCCA | CGT |
| rs518769 | CTGAGCAAAAATATATGGTGA | ACT |
| rs474215 | GTGACTCTAATATCCAAGCTA | ACT |
| AI | AAGTTTCTTCCTTTCTCCAGG | ACT |
| rs500203 | AAACATCTGCATTTTAAAC | CGT |
| rs532221 | TGTCTTCAGTATTCACCTTA | CGT |
| rs539534 | GACCAAATGACACATCTTTAAAA | ACG |
| rs531412 | AACTATATTATACATGGGTGATTT | ACG |
| rs9970514 | TCCTGATTTATTCATTGATTTTTG | ACT |
| rs568049 | GAGGAGAGGATTATCCAGTC | ACG |
| rs9727250 | TCGCGCCACTGCACTCCA | ACT |
| rs792323 | AGGCTGGAGTGCAGTGG | ACG |
| rs3765651 | TTCCTAGCCTTTACCTTT | ACT |
| rs3765654 | AAAAGAAGAGAAAATGGAATTTCT | ACT |
| rs481387 | AGGCGTATAGTAGGTGTTTAG | ACG |
| rs483259 | CTCTTATTTAAACTTGATCTCTC | ACT |
| rs2034453 | AAACTGAGAATGTTGATGGACA | ACT |
| rs7536997 | AACAACTCCAGATGCGCCG | ACG |
| rs792324 | GTCCGCACTGCCTTTAT | ACT |
| rs6660350 | CACGCAGGGGCCGCAGG | ACG |
| rs492302 | TCTCGGATCTTCCATCC | ACT |
| rs10493539 | TCATCAAGCAGTTGCTTCACA | ACT |
| rs496720 | GTAAATGCACCAATCAGCACC | ACG |
| rs570631 | TCCCATTTCCATTATTTTTTTT | CGT |
| rs4593767 | GAAGAAACAGAAAAATGTGC | ACG |
| rs576802 | TTGCATTGCTTCCAAAATGATC | CGT |
| rs518574 | AGAGGAAAAATTGCCTATG | ACT |
| rs524252 | TTCTCAAGGATATAGCTGGAG | ACG |
| rs792327 | ATGCACCAATCAGCACC | ACG |
| rs792328 | GGGAAGCTCAGGCATGG | CGT |
| rs2039407 | GTGCACTGGAGCAGTTCTG | CGT |
| rs503770 | CTGCTAAGTCTGAGTCCCAT | ACT |
| rs503904 | AAGATGCATTTATCAGATTGTATA | ACT |
| rs514012 | GTAGAGGATTGATTAGAACTGA | ACT |
| rs1412827 | TTCTAACAGTGTATTTAATCATCA | ACT |
| rs473834 | CAGAAGAGTCTCTGGGGAG | ACG |
| rs480267 | ATTTTGTTTACCAAGAAGCCTC | ACT |
| rs572180 | CTTATGCTCTTCTACCTCA | CGT |
| rs1333029 | GTTGATTTGGTTAGCAATAAT | ACG |
| rs485414 | AACATCCTAGGTCCTCT | ACT |
| rs1412823 | CCTAATTAAGGGGAAGAAGAAG | CGT |
| rs2027013 | AAGAAGAAAGGTTGAAGAC | ACT |
| rs548881 | GGTTTTGCCTAATATATTTTGATG | ACG |
| rs792329 | GACCATCCTGGCTAACA | ACG |

Genetic Analysis

Allelotyping results are shown for female (F) and male (M) cases and controls in Table 34 and Table 35, respectively. Allele frequency is noted in the fourth and fifth columns for melanoma pools and control pools, respectively.

TABLE 34
Females

| | | | | | | | | Melanoma |
|---|---|---|---|---|---|---|---|---|
| | Chromo- | Chromosome | | Female | Female | F | Odds | Associated |
| dbSNP rs# | some | Position | Alleles | Case AF | Control AF | p-Value | Ratio | Allele |
| AG | 1 | 74033656 | G/T | G = 0.167 | G = 0.106 | 0.027 | 0.593 | G |
| | | | | T = 0.832 | T = 0.893 | | | |
| rs1412825 | 1 | 74033749 | G/C | G = 0.539 | G = 0.499 | 0.269 | 0.853 | G |
| | | | | C = 0.460 | C = 0.500 | | | |
| rs4422957 | 1 | 74034084 | C/T | C = 0.484 | C = 0.427 | 0.089 | 0.796 | C |
| | | | | T = 0.515 | T = 0.572 | | | |
| rs3835390 | 1 | 74034440 | AAAAG/— | AAAAG = 0.580 | AAAAG = 0.623 | 0.199 | 1.199 | — |
| | | | | — = 0.419 | — = 0.376 | | | |
| rs944795 | 1 | 74034883 | C/G | C = 0.592 | C = 0.534 | 0.088 | 0.791 | C |
| | | | | G = 0.407 | G = 0.465 | | | |
| rs792310 | 1 | 74035428 | G/A | G = 0.508 | G = 0.566 | 0.083 | 1.265 | A |
| | | | | A = 0.491 | A = 0.433 | | | |
| rs526736 | 1 | 74035677 | A/T | A = 0.637 | A = 0.653 | 0.698 | 1.070 | T |
| | | | | T = 0.362 | T = 0.346 | | | |
| rs9804006 | 1 | 74036442 | T/C | T = 0.748 | T = 0.700 | 0.112 | 0.785 | T |
| | | | | C = 0.251 | C = 0.299 | | | |
| rs577367 | 1 | 74037621 | C/T | C = 0.540 | C = 0.460 | 0.025 | 0.727 | C |
| | | | | T = 0.459 | T = 0.539 | | | |
| rs575754 | 1 | 74037763 | T/C | T = 0.923 | T = 0.938 | 0.414 | 1.257 | C |
| | | | | C = 0.076 | C = 0.061 | | | |
| rs573721 | 1 | 74038036 | G/T | G = 0.145 | G = 0.151 | 0.861 | 1.050 | T |
| | | | | T = 0.854 | T = 0.848 | | | |
| rs487917 | 1 | 74038831 | C/T | C = 0.567 | C = 0.642 | 0.020 | 1.369 | T |
| | | | | T = 0.432 | T = 0.357 | | | |
| rs792307 | 1 | 74038935 | G/A | G = 0.530 | G = 0.617 | 0.002 | 1.431 | A |
| | | | | G = 0.495 | G = 0.597 | | | |
| rs792307 | 1 | 74038935 | G/A | A = 0.469 | A = 0.382 | 0.002 | 1.431 | A |
| | | | | A = 0.504 | A = 0.402 | | | |
| rs476350 | 1 | 74039242 | T/C | T = 0.036 | T = 0.042 | 0.729 | 1.192 | C |
| | | | | C = 0.963 | C = 0.957 | | | |
| rs9425023 | 1 | 74039846 | T/C | T = 0.595 | T = 0.626 | 0.340 | 1.142 | C |
| | | | | C = 0.404 | C = 0.373 | | | |
| rs9425024 | 1 | 74039908 | G/T | G = 0.955 | G = 0.954 | 0.976 | 0.989 | G |
| | | | | T = 0.044 | T = 0.045 | | | |
| rs571848 | 1 | 74041454 | A/G | A = 0.555 | A = 0.607 | 0.116 | 1.237 | G |
| | | | | G = 0.444 | G = 0.392 | | | |
| rs489941 | 1 | 74042258 | A/C | A = 0.564 | A = 0.465 | 0.003 | 0.672 | A |
| | | | | C = 0.435 | C = 0.534 | | | |
| rs491623 | 1 | 74042409 | A/G | A = 0.621 | A = 0.528 | 0.008 | 0.682 | A |
| | | | | G = 0.378 | G = 0.471 | | | |
| rs956 | 1 | 74042575 | T/A | T = 0.613 | T = 0.642 | 0.400 | 1.129 | A |
| | | | | A = 0.386 | A = 0.357 | | | |
| AH | 1 | 74042711 | G/A | G = 0.976 | G = 0.967 | 0.501 | 0.707 | G |
| | | | | A = 0.023 | A = 0.032 | | | |
| rs520806 | 1 | 74043276 | C/T | C = 0.586 | C = 0.643 | 0.079 | 1.276 | T |
| | | | | T = 0.413 | T = 0.356 | | | |
| rs522759 | 1 | 74043517 | T/C | T = 0.691 | T = 0.742 | 0.088 | 1.287 | C |
| | | | | C = 0.308 | C = 0.257 | | | |
| rs545664 | 1 | 74043712 | G/A | G = 0.562 | G = 0.503 | 0.074 | 0.789 | G |
| | | | | A = 0.437 | A = 0.496 | | | |
| rs545721 | 1 | 74043734 | C/T | C = 0.510 | C = 0.458 | 0.172 | 0.813 | C |
| | | | | T = 0.489 | T = 0.541 | | | |
| rs536355 | 1 | 74044111 | G/A | G = 0.534 | G = 0.465 | 0.049 | 0.758 | G |
| | | | | A = 0.465 | A = 0.534 | | | |
| rs553044 | 1 | 74044508 | G/T | G = 0.503 | G = 0.487 | 0.658 | 0.938 | G |
| | | | | T = 0.496 | T = 0.512 | | | |
| rs581353 | 1 | 74045321 | A/G | A = 0.604 | A = 0.643 | 0.281 | 1.183 | G |
| | | | | G = 0.395 | G = 0.356 | | | |
| rs496008 | 1 | 74045741 | T/A | T = 0.864 | T = 0.811 | 0.040 | 0.674 | T |
| | | | | A = 0.135 | A = 0.188 | | | |
| rs7366854 | 1 | 74046600 | C/T | C = 0.840 | C = 0.851 | 0.677 | 1.089 | T |
| | | | | T = 0.159 | T = 0.148 | | | |
| rs560808 | 1 | 74048168 | C/A | C = 0.566 | C = 0.640 | 0.033 | 1.360 | A |
| | | | | A = 0.433 | A = 0.359 | | | |
| rs485929 | 1 | 74048220 | A/G | A = 0.716 | A = 0.718 | 0.943 | 1.011 | G |
| | | | | G = 0.283 | G = 0.281 | | | |
| rs477134 | 1 | 74048849 | T/G | T = 0.560 | T = 0.624 | 0.056 | 1.306 | G |
| | | | | G = 0.439 | G = 0.375 | | | |
| rs505634 | 1 | 74049586 | G/A | G = 0.923 | G = 0.945 | 0.251 | 1.447 | A |
| | | | | A = 0.076 | A = 0.054 | | | |
| rs542136 | 1 | 74049795 | G/A | G = 0.625 | G = 0.525 | 0.007 | 0.661 | G |
| | | | | A = 0.374 | A = 0.474 | | | |
| rs545680 | 1 | 74050175 | T/C | T = 0.685 | T = 0.719 | 0.276 | 1.175 | C |
| | | | | C = 0.314 | C = 0.280 | | | |
| rs522042 | 1 | 74051017 | G/A | G = 0.699 | G = | | | |
| | | | | A = 0.301 | A = | | | |
| rs575961 | 1 | 74051145 | C/T | C = 0.979 | C = 0.948 | 0.116 | 0.390 | C |
| | | | | T = 0.020 | T = 0.051 | | | |
| rs792321 | 1 | 74051903 | A/G | A = 0.644 | A = 0.675 | 0.384 | 1.148 | G |
| | | | | G = 0.355 | G = 0.324 | | | |
| rs524306 | 1 | 74052188 | T/A | T = 0.559 | T = 0.468 | 0.005 | 0.693 | T |
| | | | | A = 0.440 | A = 0.531 | | | |
| rs495027 | 1 | 74052194 | A/C | A = 0.585 | A = 0.499 | 0.011 | 0.707 | A |
| | | | | C = 0.414 | C = 0.500 | | | |
| rs525358 | 1 | 74053239 | A/G | A = 0.150 | A = 0.141 | 0.731 | 0.930 | A |
| | | | | G = 0.849 | G = 0.858 | | | |
| rs471496 | 1 | 74053841 | G/A | G = 0.115 | G = 0.123 | 0.743 | 1.078 | A |
| | | | | A = 0.884 | A = 0.876 | | | |
| rs504866 | 1 | 74055175 | C/T | C = 0.577 | C = 0.465 | 0.002 | 0.639 | C |
| | | | | T = 0.422 | T = 0.534 | | | |
| rs532396 | 1 | 74056827 | T/C | T = 0.951 | T = 0.955 | 0.790 | 1.102 | C |
| | | | | C = 0.048 | C = 0.044 | | | |
| rs533371 | 1 | 74056947 | G/A | G = 0.528 | G = 0.484 | 0.184 | 0.840 | G |
| | | | | A = 0.471 | A = 0.515 | | | |
| rs567060 | 1 | 74058299 | A/G | A = 0.670 | A = 0.707 | 0.248 | 1.189 | G |
| | | | | G = 0.329 | G = 0.292 | | | |
| rs4650251 | 1 | 74063483 | A/G | A = 0.571 | A = 0.473 | 0.003 | 0.675 | A |
| | | | | G = 0.428 | G = 0.526 | | | |
| rs3753183 | 1 | 74069034 | G/T | G = 0.090 | G = 0.053 | 0.046 | 0.571 | G |
| | | | | T = 0.909 | T = 0.946 | | | |
| rs518769 | 1 | 74069847 | A/G | A = 0.875 | A = 0.875 | 0.996 | 1.001 | A |
| | | | | G = 0.124 | G = 0.124 | | | |
| rs474215 | 1 | 74070186 | T/C | T = 0.556 | T = 0.452 | 0.002 | 0.658 | T |
| | | | | C = 0.443 | C = 0.547 | | | |
| AI | 1 | 74071042 | T/C | T = 0.031 | T = 0.041 | 0.551 | 1.349 | C |
| | | | | C = 0.968 | C = 0.958 | | | |
| rs500203 | 1 | 74071586 | T/A | T = 0.507 | T = 0.425 | 0.016 | 0.718 | T |
| | | | | A = 0.492 | A = 0.574 | | | |
| rs532221 | 1 | 74071885 | T/A | T = 0.725 | T = 0.783 | 0.043 | 1.368 | A |
| | | | | A = 0.274 | A = 0.216 | | | |
| rs539534 | 1 | 74072672 | C/T | C = | C = | | | |
| | | | | T = | T = | | | |
| rs531412 | 1 | 74072720 | G/A | G = 0.682 | G = 0.753 | 0.034 | 1.423 | A |
| | | | | A = 0.317 | A = 0.246 | | | |
| rs9970514 | 1 | 74073693 | C/G | C = 0.421 | C = 0.474 | 0.135 | 1.241 | G |
| | | | | G = 0.578 | G = 0.525 | | | |
| rs568049 | 1 | 74074438 | C/T | C = 0.783 | C = 0.781 | 0.971 | 0.993 | C |
| | | | | T = 0.216 | T = 0.218 | | | |
| rs9727250 | 1 | 74077515 | T/C | T = 0.074 | T = 0.057 | 0.336 | 0.758 | T |
| | | | | C = 0.925 | C = 0.942 | | | |
| rs792323 | 1 | 74077529 | C/T | C = 0.815 | C = 0.817 | 0.933 | 1.014 | T |
| | | | | T = 0.462 | T = 0.436 | | | |
| rs3765651 | 1 | 74079297 | A/C | A = 0.666 | A = 0.710 | 0.174 | 1.232 | C |
| | | | | C = 0.333 | C = 0.289 | | | |
| rs3765654 | 1 | 74079646 | C/G | C = 0.799 | C = 0.686 | ˜0.001 | 0.551 | C |
| | | | | G = 0.200 | G = 0.313 | | | |
| rs481387 | 1 | 74080385 | C/T | C = 0.517 | C = 0.447 | 0.048 | 0.755 | C |
| | | | | T = 0.482 | T = 0.552 | | | |
| rs483259 | 1 | 74080564 | T/C | T = 0.580 | T = 0.665 | 0.015 | 1.438 | C |
| | | | | C = 0.419 | C = 0.334 | | | |
| rs2034453 | 1 | 74082116 | A/G | A = 0.208 | A = 0.143 | 0.015 | 0.634 | A |
| | | | | G = 0.791 | G = 0.856 | | | |
| rs7536997 | 1 | 74082887 | G/A | G = | G = | | | |
| | | | | A = | A = | | | |
| rs792324 | 1 | 74083006 | T/C | T = 0.067 | T = 0.053 | 0.496 | 0.779 | T |
| | | | | T = 0.038 | T = 0.036 | | | |
| rs792324 | 1 | 74083006 | T/C | C = 0.932 | C = 0.946 | 0.496 | 0.779 | T |
| | | | | C = 0.961 | C = 0.963 | | | |
| rs6660350 | 1 | 74083486 | C/T | C = 0.191 | C = 0.126 | 0.010 | 0.607 | C |
| | | | | T = 0.808 | T = 0.873 | | | |
| rs492302 | 1 | 74084074 | G/C | G = | G = 0.389 | | | |
| | | | | C = | C = 0.611 | | | |
| rs10493539 | 1 | 74085512 | A/G | A = 0.375 | A = 0.330 | 0.175 | 0.821 | A |
| | | | | G = 0.624 | G = 0.669 | | | |
| rs496720 | 1 | 74088648 | G/A | G = 0.964 | G = 0.977 | 0.394 | 1.598 | A |
| | | | | A = 0.035 | A = 0.022 | | | |
| rs570631 | 1 | 74089790 | T/A | T = 0.375 | T = 0.392 | 0.688 | 1.074 | A |
| | | | | A = 0.624 | A = 0.607 | | | |
| rs4593767 | 1 | 74093028 | G/A | G = 0.231 | G = 0.270 | 0.182 | 1.234 | A |
| | | | | A = 0.768 | A = 0.729 | | | |
| rs576802 | 1 | 74094231 | T/A | T = 0.860 | T = 0.846 | 0.569 | 0.898 | T |
| | | | | A = 0.139 | A = 0.153 | | | |
| rs518574 | 1 | 74100973 | A/G | A = 0.972 | A = 0.969 | 0.861 | 0.920 | A |
| | | | | G = 0.027 | G = 0.030 | | | |
| rs524252 | 1 | 74102532 | C/T | C = 0.828 | C = 0.826 | 0.925 | 0.983 | C |
| | | | | T = 0.171 | T = 0.173 | | | |
| rs792327 | 1 | 74103456 | G/A | G = 0.957 | G = 0.960 | 0.893 | 1.070 | A |
| | | | | A = 0.043 | A = 0.040 | | | |
| rs792328 | 1 | 74103887 | C/A | C = | C = 0.337 | | | |
| | | | | A = | A = 0.663 | | | |
| rs2039407 | 1 | 74104305 | A/T | A = 0.930 | A = 0.962 | 0.049 | 1.924 | T |
| | | | | T = 0.069 | T = 0.037 | | | |
| rs503770 | 1 | 74105381 | T/C | T = 0.853 | T = 0.816 | 0.137 | 0.764 | T |
| | | | | C = 0.146 | C = 0.183 | | | |
| rs503904 | 1 | 74105440 | A/G | A = 0.838 | A = 0.809 | 0.269 | 0.820 | A |
| | | | | G = 0.161 | G = 0.190 | | | |
| rs514012 | 1 | 74107016 | A/G | A = 0.860 | A = 0.811 | 0.062 | 0.698 | A |
| | | | | G = 0.139 | G = 0.188 | | | |
| rs1412827 | 1 | 74107532 | A/G | A = 0.385 | A = 0.327 | 0.091 | 0.777 | A |
| | | | | G = 0.614 | G = 0.672 | | | |
| rs473834 | 1 | 74112969 | G/A | G = 0.142 | G = 0.191 | 0.050 | 1.425 | A |
| | | | | A = 0.857 | A = 0.808 | | | |
| rs480267 | 1 | 74113659 | A/G | A = 0.317 | A = 0.356 | 0.273 | 1.190 | G |
| | | | | G = 0.682 | G = 0.643 | | | |
| rs572180 | 1 | 74119134 | G/T | G = 0.868 | G = 0.838 | 0.202 | 0.786 | G |
| | | | | T = 0.131 | T = 0.161 | | | |
| rs1333029 | 1 | 74119257 | G/A | G = 0.945 | G = 0.971 | 0.088 | 1.948 | A |
| | | | | A = 0.054 | A = 0.028 | | | |
| rs485414 | 1 | 74119377 | A/G | A = 0.911 | A = 0.906 | 0.824 | 0.947 | A |
| | | | | G = 0.088 | G = 0.093 | | | |
| rs1412823 | 1 | 74119701 | A/T | A = 0.774 | A = 0.779 | 0.876 | 1.030 | T |
| | | | | T = 0.225 | T = 0.220 | | | |
| rs2027013 | 1 | 74120888 | T/G | T = 0.148 | T = 0.075 | 0.005 | 0.470 | T |
| | | | | G = 0.852 | G = 0.925 | | | |
| rs548881 | 1 | 74128480 | C/T | C = 0.898 | C = 0.862 | 0.176 | 0.709 | C |
| | | | | T = 0.101 | T = 0.137 | | | |
| rs792329 | 1 | 74130625 | C/T | C = 0.732 | C = 0.744 | 0.688 | 1.070 | T |
| | | | | T = 0.268 | T = 0.256 | | | |

TABLE 35
Males

| | | | | | | | | Melanoma |
|---|---|---|---|---|---|---|---|---|
| | Chromo- | Chromosome | | Male | Male | M | Odds | Associated |
| dbSNP rs# | some | Position | Alleles | Case AF | Control AF | p-Value | Ratio | Allele |
| AG | 1 | 74033656 | G/T | G = 0.136 | G = 0.103 | 0.178 | 0.730 | G |
| | | | | T = 0.863 | T = 0.896 | | | |
| rs1412825 | 1 | 74033749 | G/C | G = 0.520 | G = 0.518 | 0.947 | 0.990 | G |
| | | | | C = 0.479 | C = 0.481 | | | |
| rs4422957 | 1 | 74034084 | C/T | C = 0.462 | C = 0.450 | 0.739 | 0.955 | C |
| | | | | T = 0.537 | T = 0.549 | | | |
| rs3835390 | 1 | 74034440 | AAAAG/— | AAAAG = 0.618 | AAAAG = 0.599 | 0.588 | 0.924 | A |
| | | | | — = 0.381 | — = 0.400 | | | |
| rs944795 | 1 | 74034883 | C/G | C = 0.553 | C = 0.540 | 0.707 | 0.948 | C |
| | | | | G = 0.446 | G = 0.459 | | | |
| rs792310 | 1 | 74035428 | G/A | G = 0.548 | G = 0.564 | 0.630 | 1.069 | A |
| | | | | A = 0.451 | A = 0.435 | | | |
| rs526736 | 1 | 74035677 | A/T | A = | A = | | | |
| | | | | T = | T = | | | |
| rs9804006 | 1 | 74036442 | T/C | T = 0.724 | T = 0.691 | 0.306 | 0.853 | T |
| | | | | C = 0.275 | C = 0.308 | | | |
| rs577367 | 1 | 74037621 | C/T | C = | C = | | | |
| | | | | T = | T = | | | |
| rs575754 | 1 | 74037763 | T/C | T = 0.929 | T = 0.946 | 0.343 | 1.346 | C |
| | | | | C = 0.070 | C = 0.053 | | | |
| rs573721 | 1 | 74038036 | G/T | G = 0.094 | G = 0.137 | 0.210 | 1.525 | T |
| | | | | T = 0.905 | T = 0.862 | | | |
| rs487917 | 1 | 74038831 | C/T | C = 0.629 | C = 0.684 | 0.159 | 1.282 | T |
| | | | | T = 0.370 | T = 0.315 | | | |
| rs792307 | 1 | 74038935 | G/A | G = 0.591 | G = 0.654 | 0.286 | 1.306 | A |
| | | | | G = 0.543 | G = 0.583 | | | |
| rs792307 | 1 | 74038935 | G/A | A = 0.408 | A = 0.345 | 0.286 | 1.306 | A |
| | | | | A = 0.456 | A = 0.416 | | | |
| rs476350 | 1 | 74039242 | T/C | T = 0.052 | T = 0.047 | 0.767 | 0.886 | T |
| | | | | C = 0.947 | C = 0.952 | | | |
| rs9425023 | 1 | 74039846 | T/C | T = 0.614 | T = 0.594 | 0.577 | 0.919 | T |
| | | | | C = 0.385 | C = 0.405 | | | |
| rs9425024 | 1 | 74039908 | G/T | G = 0.953 | G = 0.981 | 0.138 | 2.541 | T |
| | | | | T = 0.047 | T = 0.019 | | | |
| rs571848 | 1 | 74041454 | A/G | A = 0.600 | A = 0.602 | 0.944 | 1.010 | G |
| | | | | G = 0.399 | G = 0.397 | | | |
| rs489941 | 1 | 74042258 | A/C | A = 0.500 | A = 0.481 | 0.601 | 0.928 | A |
| | | | | C = 0.499 | C = 0.518 | | | |
| rs491623 | 1 | 74042409 | A/G | A = 0.577 | A = 0.565 | 0.771 | 0.954 | A |
| | | | | G = 0.422 | G = 0.434 | | | |
| rs956 | 1 | 74042575 | T/A | T = 0.624 | T = 0.627 | 0.939 | 1.011 | A |
| | | | | A = 0.375 | A = 0.372 | | | |
| AH | 1 | 74042711 | G/A | G = 0.966 | G = 0.961 | 0.762 | 0.878 | G |
| | | | | A = 0.033 | A = 0.038 | | | |
| rs520806 | 1 | 74043276 | C/T | C = 0.631 | C = 0.664 | 0.324 | 1.155 | T |
| | | | | T = 0.368 | T = 0.335 | | | |
| rs522759 | 1 | 74043517 | T/C | T = 0.724 | T = 0.752 | 0.361 | 1.154 | C |
| | | | | C = 0.275 | C = 0.247 | | | |
| rs545664 | 1 | 74043712 | G/A | G = 0.534 | G = 0.524 | 0.794 | 0.961 | G |
| | | | | A = 0.465 | A = 0.475 | | | |
| rs545721 | 1 | 74043734 | C/T | C = 0.534 | C = 0.479 | 0.182 | 0.803 | C |
| | | | | T = 0.465 | T = 0.520 | | | |
| rs536355 | 1 | 74044111 | G/A | G = | G = | | | |
| | | | | A = | A = | | | |
| rs553044 | 1 | 74044508 | G/T | G = 0.471 | G = 0.490 | 0.594 | 1.079 | T |
| | | | | T = 0.528 | T = 0.509 | | | |
| rs581353 | 1 | 74045321 | A/G | A = 0.632 | A = 0.647 | 0.666 | 1.064 | G |
| | | | | G = 0.367 | G = 0.352 | | | |
| rs496008 | 1 | 74045741 | T/A | T = 0.824 | T = 0.822 | 0.947 | 0.988 | T |
| | | | | A = 0.175 | A = 0.177 | | | |
| rs7366854 | 1 | 74046600 | C/T | C = 0.847 | C = 0.854 | 0.788 | 1.057 | T |
| | | | | T = 0.152 | T = 0.145 | | | |
| rs560808 | 1 | 74048168 | C/A | C = 0.593 | C = 0.614 | 0.530 | 1.093 | A |
| | | | | A = 0.406 | A = 0.385 | | | |
| rs485929 | 1 | 74048220 | A/G | A = 0.692 | A = 0.715 | 0.481 | 1.113 | G |
| | | | | G = 0.307 | G = 0.284 | | | |
| rs477134 | 1 | 74048849 | T/G | T = 0.587 | T = 0.612 | 0.464 | 1.110 | G |
| | | | | G = 0.412 | G = 0.387 | | | |
| rs505634 | 1 | 74049586 | G/A | G = 0.939 | G = 0.948 | 0.664 | 1.177 | A |
| | | | | A = 0.060 | A = 0.051 | | | |
| rs542136 | 1 | 74049795 | G/A | G = | G = | | | |
| | | | | A = | A = | | | |
| rs545680 | 1 | 74050175 | T/C | T = 0.707 | T = 0.714 | 0.826 | 1.035 | C |
| | | | | C = 0.292 | C = 0.285 | | | |
| rs522042 | 1 | 74051017 | G/A | G = | G = | | | |
| | | | | A = | A = | | | |
| rs575961 | 1 | 74051145 | C/T | C = 0.979 | C = 0.963 | 0.261 | 0.547 | C |
| | | | | T = 0.020 | T = 0.036 | | | |
| rs792321 | 1 | 74051903 | A/G | A = 0.667 | A = 0.672 | 0.877 | 1.025 | G |
| | | | | G = 0.332 | G = 0.327 | | | |
| rs524306 | 1 | 74052188 | T/A | T = 0.517 | T = 0.478 | 0.260 | 0.854 | T |
| | | | | A = 0.482 | A = 0.521 | | | |
| rs495027 | 1 | 74052194 | A/C | A = 0.562 | A = 0.500 | 0.082 | 0.778 | A |
| | | | | C = 0.437 | C = 0.499 | | | |
| rs525358 | 1 | 74053239 | A/G | A = 0.127 | A = 0.132 | 0.833 | 1.050 | G |
| | | | | G = 0.872 | G = 0.867 | | | |
| rs471496 | 1 | 74053841 | G/A | G = 0.097 | G = 0.096 | 0.984 | 0.994 | G |
| | | | | A = 0.902 | A = 0.903 | | | |
| rs504866 | 1 | 74055175 | C/T | C = 0.604 | C = 0.506 | 0.032 | 0.672 | C |
| | | | | T = 0.395 | T = 0.493 | | | |
| rs532396 | 1 | 74056827 | T/C | T = 0.960 | T = 0.963 | 0.879 | 1.070 | C |
| | | | | C = 0.039 | C = 0.036 | | | |
| rs533371 | 1 | 74056947 | G/A | G = 0.510 | G = 0.510 | 0.986 | 1.002 | G |
| | | | | A = 0.489 | A = 0.489 | | | |
| rs567060 | 1 | 74058299 | A/G | A = 0.699 | A = 0.697 | 0.966 | 0.993 | A |
| | | | | G = 0.300 | G = 0.302 | | | |
| rs4650251 | 1 | 74063483 | A/G | A = 0.521 | A = 0.477 | 0.213 | 0.839 | A |
| | | | | G = 0.478 | G = 0.522 | | | |
| rs3753183 | 1 | 74069034 | G/T | G = 0.080 | G = 0.042 | 0.031 | 0.503 | G |
| | | | | T = 0.919 | T = 0.957 | | | |
| rs518769 | 1 | 74069847 | A/G | A = 0.873 | A = 0.870 | 0.908 | 0.974 | A |
| | | | | G = 0.126 | G = 0.129 | | | |
| rs474215 | 1 | 74070186 | T/C | T = 0.485 | T = 0.434 | 0.134 | 0.813 | T |
| | | | | C = 0.514 | C = 0.565 | | | |
| AI | 1 | 74071042 | T/C | T = 0.048 | T = 0.031 | 0.381 | 0.635 | T |
| | | | | C = 0.951 | C = 0.968 | | | |
| rs500203 | 1 | 74071586 | T/A | T = 0.453 | T = 0.423 | 0.381 | 0.885 | T |
| | | | | A = 0.546 | A = 0.576 | | | |
| rs532221 | 1 | 74071885 | T/A | T = 0.722 | T = 0.822 | ˜0.001 | 1.776 | A |
| | | | | A = 0.277 | A = 0.177 | | | |
| rs539534 | 1 | 74072672 | C/T | C = 0.536 | C = 0.576 | 0.353 | 1.174 | T |
| | | | | T = 0.463 | T = 0.423 | | | |
| rs531412 | 1 | 74072720 | G/A | G = 0.740 | G = 0.765 | 0.525 | 1.141 | A |
| | | | | A = 0.259 | A = 0.234 | | | |
| rs9970514 | 1 | 74073693 | C/G | C = 0.443 | C = 0.462 | 0.576 | 1.083 | G |
| | | | | G = 0.556 | G = 0.537 | | | |
| rs568049 | 1 | 74074438 | C/T | C = 0.773 | C = 0.777 | 0.914 | 1.023 | T |
| | | | | T = 0.226 | T = 0.222 | | | |
| rs9727250 | 1 | 74077515 | T/C | T = 0.073 | T = 0.059 | 0.499 | 0.796 | T |
| | | | | C = 0.926 | C = 0.940 | | | |
| rs792323 | 1 | 74077529 | C/T | C = 0.820 | C = 0.824 | 0.881 | 1.029 | T |
| | | | | T = 0.434 | T = 0.406 | | | |
| rs3765651 | 1 | 74079297 | A/C | A = 0.704 | A = 0.704 | 0.996 | 0.999 | C |
| | | | | C = 0.295 | C = 0.295 | | | |
| rs3765654 | 1 | 74079646 | C/G | C = 0.745 | C = 0.673 | 0.031 | 0.705 | C |
| | | | | G = 0.254 | G = 0.326 | | | |
| rs481387 | 1 | 74080385 | C/T | C = 0.479 | C = 0.462 | 0.622 | 0.934 | C |
| | | | | T = 0.520 | T = 0.537 | | | |
| rs483259 | 1 | 74080564 | T/C | T = 0.636 | T = 0.660 | 0.478 | 1.110 | C |
| | | | | C = 0.363 | C = 0.339 | | | |
| rs2034453 | 1 | 74082116 | A/G | A = 0.172 | A = 0.150 | 0.463 | 0.852 | A |
| | | | | G = 0.827 | G = 0.849 | | | |
| rs7536997 | 1 | 74082887 | G/A | G = 0.947 | G = 0.955 | 0.727 | 1.194 | A |
| | | | | A = 0.052 | A = 0.044 | | | |
| rs792324 | 1 | 74083006 | T/C | T = 0.070 | T = 0.060 | 0.633 | 0.850 | T |
| | | | | T = 0.037 | T = 0.044 | | | |
| rs792324 | 1 | 74083006 | T/C | C = 0.929 | C = 0.939 | 0.633 | 0.850 | T |
| | | | | C = 0.962 | C = 0.955 | | | |
| rs6660350 | 1 | 74083486 | C/T | C = 0.192 | C = 0.159 | 0.259 | 0.792 | C |
| | | | | T = 0.807 | T = 0.840 | | | |
| rs492302 | 1 | 74084074 | G/C | G = | G = | | | |
| | | | | C = | C = | | | |
| rs10493539 | 1 | 74085512 | A/G | A = 0.385 | A = 0.322 | 0.124 | 0.759 | A |
| | | | | G = 0.614 | G = 0.677 | | | |
| rs496720 | 1 | 74088648 | G/A | G = 0.960 | G = 0.969 | 0.635 | 1.285 | A |
| | | | | A = 0.039 | A = 0.030 | | | |
| rs570631 | 1 | 74089790 | T/A | T = 0.384 | T = 0.373 | 0.769 | 0.953 | T |
| | | | | A = 0.615 | A = 0.626 | | | |
| rs4593767 | 1 | 74093028 | G/A | G = 0.300 | G = 0.260 | 0.236 | 0.821 | G |
| | | | | A = 0.699 | A = 0.739 | | | |
| rs576802 | 1 | 74094231 | T/A | T = 0.833 | T = 0.865 | 0.209 | 1.282 | A |
| | | | | A = 0.166 | A = 0.134 | | | |
| rs518574 | 1 | 74100973 | A/G | A = 0.966 | A = 0.968 | 0.880 | 1.069 | G |
| | | | | G = 0.033 | G = 0.031 | | | |
| rs524252 | 1 | 74102532 | C/T | C = 0.839 | C = 0.840 | 0.966 | 1.008 | T |
| | | | | T = 0.160 | T = 0.159 | | | |
| rs792327 | 1 | 74103456 | G/A | G = 0.963 | G = 0.978 | 0.477 | 1.720 | A |
| | | | | A = 0.037 | A = 0.022 | | | |
| rs792328 | 1 | 74103887 | C/A | C = | C = | | | |
| | | | | A = | A = | | | |
| rs2039407 | 1 | 74104305 | A/T | A = 0.945 | A = 0.942 | 0.872 | 0.937 | A |
| | | | | T = 0.054 | T = 0.057 | | | |
| rs503770 | 1 | 74105381 | T/C | T = 0.824 | T = 0.825 | 0.946 | 1.013 | C |
| | | | | C = 0.175 | C = 0.174 | | | |
| rs503904 | 1 | 74105440 | A/G | A = 0.814 | A = 0.809 | 0.870 | 0.970 | A |
| | | | | G = 0.185 | G = 0.190 | | | |
| rs514012 | 1 | 74107016 | A/G | A = 0.821 | A = 0.801 | 0.473 | 0.880 | A |
| | | | | G = 0.178 | G = 0.198 | | | |
| rs1412827 | 1 | 74107532 | A/G | A = 0.365 | A = 0.387 | 0.544 | 1.101 | G |
| | | | | G = 0.634 | G = 0.612 | | | |
| rs473834 | 1 | 74112969 | G/A | G = 0.178 | G = 0.185 | 0.803 | 1.046 | A |
| | | | | A = 0.821 | A = 0.814 | | | |
| rs480267 | 1 | 74113659 | A/G | A = 0.332 | A = 0.364 | 0.439 | 1.153 | G |
| | | | | G = 0.667 | G = 0.635 | | | |
| rs572180 | 1 | 74119134 | G/T | G = 0.845 | G = 0.850 | 0.816 | 1.047 | T |
| | | | | T = 0.154 | T = 0.149 | | | |
| rs1333029 | 1 | 74119257 | G/A | G = 0.961 | G = 0.977 | 0.294 | 1.732 | A |
| | | | | A = 0.038 | A = 0.022 | | | |
| rs485414 | 1 | 74119377 | A/G | A = 0.897 | A = 0.923 | 0.233 | 1.376 | G |
| | | | | G = 0.102 | G = 0.076 | | | |
| rs1412823 | 1 | 74119701 | A/T | A = 0.761 | A = 0.761 | 0.995 | 0.999 | T |
| | | | | T = 0.238 | T = 0.238 | | | |
| rs2027013 | 1 | 74120888 | T/G | T = 0.093 | T = | | | |
| | | | | G = 0.907 | G = | | | |
| rs548881 | 1 | 74128480 | C/T | C = 0.920 | C = 0.898 | 0.361 | 0.768 | C |
| | | | | T = 0.079 | T = 0.101 | | | |
| rs792329 | 1 | 74130625 | C/T | C = 0.754 | C = 0.756 | 0.951 | 1.010 | T |
| | | | | T = 0.246 | T = 0.244 | | | |

Allelotyping results were considered significant with a calculated p-value of less than or equal to 0.05 for allelotype results. These values are indicated in bold. The assay failed for those SNPs in which the allele frequency is blank. The combined allelotyping p-values for males and females were plotted in FIG. 5A and separately for females and males in FIGS. 5B and 5C, respectively. The position of each SNP on the chromosome is presented on the x-axis. The y-axis gives the negative logarithm (base 10) of the p-value comparing the estimated allele frequency in the case group to that of the control group. The minor allele frequency of the control group for each SNP designated by an X or other symbol on the graphs in FIGS. 5A, 5B and 5C can be determined by consulting Table 34 or 35. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brooks/Cole.). The black line (or generally top-most curve, e.g., see peak in left-most graph just to the left of position 92150000) provides a local test for excess statistical significance to identify regions of association. This was created by use of a 10 kb sliding window with 1 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 10⁻⁸ were truncated at that value.

Finally, the exons and introns of the genes in the covered region are plotted below each graph at the appropriate chromosomal positions. The gene boundary is indicated by the broken horizontal line. The exon positions are shown as thick, unbroken bars. An arrow is place at the 3′ end of each gene to show the direction of transcription.

### Example 10

### REPS2 Proximal SNPs

It has been discovered that a polymorphic variation (rs1904528) in the untranslated region of a gene encoding presynaptic cytomatrix protein (REPS2) is associated with the occurrence of melanoma. See Table 3. Subsequently, one-hundred-three allelic variants located within or nearby the target genes were identified and subsequently allelotyped in melanoma case and control sample sets as described in Examples 1 and 2. Several SNPs, designated by SNP_ID “AJ”, “AK”, and “AL” in column 1 of Table 36, are novel and were identified through SNP discovery methods using dye terminator sequencing. Methods of diedeoxynucleotide chain termination chemistry derived sequencing are described by Sanger et al. (Proc. Nat. Acad. Sci. USA 74, 5463-5467, 1997). Briefly, terminators are labeled with fluorescent dyes for automated detection. Fluorescein is measured by an argon ion laser in the sequencing instrument, which transfers light energy to a rhodamine acceptor dye. Each acceptor dye emits light at a characteristic wavelength for detection, identifying the nucleotide that terminated extension of the DNA fragment. Novel SNPs are identified when sequence from multiple samples are compared on one or more strands. The forward and reverse primers used to identify AJ are GCTTTAGGATAGGGAGGAATGG and CCCAGCGCCAGGTAACTAAC, respectively. The forward and reverse primers used to identify AL are CCACCCTTCCTCTGACTGTT and ACAACCCCATCAAAAAGTGG, respectively. The polymorphic variants are set forth in Table 36. The chromosome position provided in column four of Table 36 is based on Genome “Build 34” of NCBI's GenBank and dbSNP build “119”.

TABLE 36

| | | Position | | | Allele | |
|---|---|---|---|---|---|---|
| | Chromo- | in SEQ | Chromosome | Allele | Present in | IUPAC |
| dbSNP rs# | some | ID NO: 7 | Position | Variants | SEQ ID NO: 7 | Code |
| rs1904525 | X | 223 | 16315373 | T/C | C | Y |
| rs5924557 | X | 790 | 16315940 | C/T | C | Y |
| rs5924616 | X | 834 | 16315984 | A/T | T | W |
| rs5924617 | X | 1,167 | 16316317 | G/A | G | R |
| rs7883612 | X | 1,372 | 16316522 | G/A | G | R |
| rs5924618 | X | 2,448 | 16317598 | C/T | C | Y |
| rs5924619 | X | 5,291 | 16320441 | T/C | T | Y |
| AJ | X | 10,537 | 16325687 | G/T | T | K |
| rs5901596 | X | 13,495 | 16328645 | —/TGG | G | N |
| rs7878645 | X | 13,861 | 16329011 | T/C | C | Y |
| rs5924564 | X | 14,299 | 16329449 | C/G | C | S |
| rs5924535 | X | 23,962 | 16339112 | A/G | G | R |
| AK | X | 25,216 | 16340366 | A/C | A | M |
| rs6629197 | X | 33,304 | 16348454 | C/T | T | Y |
| rs7060904 | X | 40,831 | 16355981 | G/T | T | K |
| rs4828523 | X | 41,424 | 16356574 | A/G | G | R |
| rs5924568 | X | 41,898 | 16357048 | C/G | G | S |
| rs5924569 | X | 41,984 | 16357134 | A/G | A | R |
| rs1904528 | X | 50,013 | 16365163 | C/T | G | R |
| rs5924570 | X | 50,632 | 16365782 | C/G | G | S |
| rs1904527 | X | 54,491 | 16369641 | A/G | C | Y |
| rs2052763 | X | 55,422 | 16370572 | G/A | C | Y |
| rs1549529 | X | 62,416 | 16377566 | A/G | A | R |
| rs5924573 | X | 64,172 | 16379322 | A/G | A | R |
| rs5901600 | X | 69,271 | 16384421 | —/T | T | N |
| rs2288304 | X | 70,244 | 16385394 | T/C | T | Y |
| rs4240072 | X | 71,687 | 16386837 | A/G | T | Y |
| rs3055191 | X | 73,571 | 16388721 | —/ | G | N |
| rs6632943 | X | 78,150 | 16393300 | T/C | C | Y |
| rs5924580 | X | 80,031 | 16395181 | G/A | A | R |
| rs4828544 | X | 82,908 | 16398058 | T/C | C | Y |
| rs1807412 | X | 85,218 | 16400368 | A/G | C | Y |
| rs2010456 | X | 85,269 | 16400419 | G/A | G | R |
| rs5969758 | X | 87,416 | 16402566 | G/A | G | R |
| rs1965051 | X | 89,285 | 16404435 | A/G | T | Y |
| re9887218 | X | 89,644 | 16404794 | T/C | C | Y |
| rs1426800 | X | 92,647 | 16407797 | G/A | T | Y |
| rs5969759 | X | 102,294 | 16417444 | A/G | G | R |
| rs6527766 | X | 103,071 | 16418221 | T/C | C | Y |
| rs1365529 | X | 106,614 | 16421764 | T/C | C | Y |
| rs5924582 | X | 107,231 | 16422381 | G/A | A | R |
| rs2114458 | X | 109,076 | 16424226 | G/A | A | R |
| rs5924583 | X | 109,520 | 16424670 | T/C | C | Y |
| rs5924545 | X | 111,210 | 16426360 | G/A | A | R |
| rs5969761 | X | 113,905 | 16429055 | C/T | T | Y |
| rs6629201 | X | 114,439 | 16429589 | C/T | C | Y |
| rs6629202 | X | 114,544 | 16429694 | C/T | T | Y |
| rs5924584 | X | 114,886 | 16430036 | A/G | G | R |
| rs5924585 | X | 115,261 | 16430411 | A/G | G | R |
| rs933946 | X | 116,964 | 16432114 | T/C | G | R |
| AL | X | 119,368 | 16434518 | C/T | C | Y |
| rs5969762 | X | 122,315 | 16437465 | A/G | G | R |
| rs713355 | X | 126,768 | 16441918 | T/C | G | R |
| rs1365530 | X | 127,291 | 16442441 | G/C | C | S |
| rs1961490 | X | 127,365 | 16442515 | A/T | A | W |
| rs6527769 | X | 128,866 | 16444016 | C/G | C | S |
| rs2006847 | X | 133,171 | 16448321 | A/G | G | R |
| rs2382813 | X | 136,375 | 16451525 | A/G | G | R |
| rs2033120 | X | 136,531 | 16451681 | C/G | C | S |
| rs2162394 | X | 136,895 | 16452045 | A/G | T | Y |
| rs1346246 | X | 138,428 | 16453578 | C/A | G | K |
| rs1365527 | X | 139,052 | 16454202 | G/C | G | S |
| rs1426801 | X | 139,255 | 16454405 | T/C | G | R |
| rs5924546 | X | 140,611 | 16455761 | G/C | C | S |
| rs5924547 | X | 142,144 | 16457294 | G/C | C | S |
| rs1365528 | X | 144,730 | 16459880 | G/A | C | Y |
| rs4828524 | X | 147,464 | 16462614 | A/G | A | R |
| rs5924588 | X | 148,435 | 16463585 | C/T | T | Y |
| rs5969764 | X | 154,005 | 16469155 | C/T | T | Y |
| rs5924589 | X | 158,068 | 16473218 | T/G | T | K |
| rs5924590 | X | 160,286 | 16475436 | G/T | T | K |
| rs5924591 | X | 163,778 | 16478928 | G/T | T | K |
| rs5924592 | X | 165,697 | 16480847 | A/G | A | R |
| rs5924548 | X | 165,741 | 16480891 | G/A | G | R |
| rs5924593 | X | 165,785 | 16480935 | C/G | G | S |
| rs4828547 | X | 167,511 | 16482661 | A/G | G | R |
| rs714070 | X | 168,378 | 16483528 | A/G | C | Y |
| rs5969768 | X | 175,465 | 16490615 | T/C | C | Y |
| rs4828526 | X | 176,485 | 16491635 | G/A | G | R |
| rs5969738 | X | 177,613 | 16492763 | G/A | A | R |
| rs5924595 | X | 178,208 | 16493358 | G/A | A | R |
| rs5924549 | X | 178,275 | 16493425 | A/G | G | R |
| rs4828528 | X | 179,554 | 16494704 | T/C | C | Y |
| rs5969769 | X | 180,382 | 16495532 | G/A | A | R |
| rs4007744 | X | 182,507 | 16497657 | C/T | T | Y |
| rs2891073 | X | 184,214 | 16499364 | T/C | C | Y |
| rs5924599 | X | 185,900 | 16501050 | C/G | G | S |
| rs1549531 | X | 191,011 | 16506161 | G/A | A | R |
| rs5924601 | X | 192,972 | 16508122 | A/G | G | R |
| rs5901609 | X | 195,135 | 16510285 | C/— | C | N |
| rs5969739 | X | 196,266 | 16511416 | C/T | C | Y |
| rs5924602 | X | 197,399 | 16512549 | T/G | T | K |
| rs1426798 | X | 198,610 | 16513760 | A/C | A | M |
| rs7891150 | X | 198,851 | 16514001 | C/T | T | Y |
| rs4828550 | X | 199,052 | 16514202 | G/A | A | R |
| rs5924603 | X | 200,639 | 16515789 | T/C | T | Y |
| rs1807947 | X | 209,336 | 16524486 | A/G | T | Y |
| rs954253 | X | 209,379 | 16524529 | A/G | A | R |
| rs2382814 | X | 210,011 | 16525161 | T/G | T | K |
| rs1896178 | X | 210,380 | 16525530 | A/G | C | Y |
| rs1896177 | X | 210,400 | 16525550 | A/G | T | Y |
| rs5924605 | X | 210,554 | 16525704 | C/G | G | S |
| rs5924606 | X | 213,071 | 16528221 | A/G | A | R |

Assay for Verifying and Allelotyping SNPs

The methods used to verify and allelotype the one-hundred-three proximal SNPs of Table 36 are the same methods described in Examples 1 and 2 herein. The primers and probes used in these assays are provided in Table 37 and Table 38, respectively.

TABLE 37

| | | |
|---|---|---|
| dbSNP rs # | Forward PCR primer | Reverse PCR primer |

| | | |
|---|---|---|
| rs1904525 | CAAGTTACTGTCTCAGCCTG | CAAGTTACTGTCTCAGCCTG |
| rs5924557 | ACACTCAGGCTACTTCTTAC | ACACTCAGGCTACTTCTTAC |
| rs5924616 | AGCACACGTAATTTGGGTCC | AGCACACGTAATTTGGGTCC |
| rs5924617 | AAAGAGCAGTTCCCCTACAC | AAAGAGCAGTTCCCCTACAC |
| rs7883612 | CCTCCATGATATGGTTTGTC | CCTCCATGATATGGTTTGTC |
| rs5924618 | TGATCCTCCCACCTCAGGTT | TGATCCTCCCACCTCAGGTT |
| rs5924619 | AGATGGCTGTTGACTGAGAG | AGATGGCTGTTGACTGAGAG |
| AJ | CAGAGTGTTCAGGGTGGTAT | CAGAGTGTTCAGGGTGGTAT |
| rs5901596 | AAAGTCAGCAACCAGCAGAC | AAAGTCAGCAACCAGCAGAC |
| rs7878645 | TTGCAGTGAGCCAAGATCAC | TTGCAGTGAGCCAAGATCAC |
| rs5924564 | GATGAACCTACATTGACACC | GATGAACCTACATTGACACC |
| rs5924535 | AGGTTGCTATGGGAATGGAG | AGGTTGCTATGGGAATGGAG |
| AK | GGGACAGTTTTCTTGATTTCC | GGGACAGTTTTCTTGATTTCC |
| rs6629197 | ACTTCCCAGACGGGATGGCT | ACTTCCCAGACGGGATGGCT |
| rs7060904 | CCAATTGGTAGACAACCAAG | CCAATTGGTAGACAACCAAG |
| rs4828523 | GAGTCTCCTGCTCTTGAAAG | GAGTCTCCTGCTCTTGAAAG |
| rs5924568 | GATGCGATCACTCACGTCTG | GATGCGATCACTCACGTCTG |
| rs5924569 | ACATAGGAAGACCCCCATCT | ACATAGGAAGACCCCCATCT |
| rs1904528 | GCTATCTCTTTCACATTGCTC | GCTATCTCTTTCACATTGCTC |
| rs5924570 | TCTAATATGTGTCGGCTCTG | TCTAATATGTGTCGGCTCTG |
| rs1904527 | TCCATCCAGATCATTGCATG | TCCATCCAGATCATTGCATG |
| rs2052763 | TGTTATGAAATGGAGCCACG | TGTTATGAAATGGAGCCACG |
| rs1549529 | AGGATAGTAATGGCCTGGTG | AGGATAGTAATGGCCTGGTG |
| rs5924573 | GGGAGTAGCTTACATCTTGG | GGGAGTAGCTTACATCTTGG |
| rs5901600 | CATACCAAGGATTACAATGTC | CATACCAAGGATTACAATGTC |
| rs2288304 | GGGAGTGGATTCTAATGTTC | GGGAGTGGATTCTAATGTTC |
| rs4240072 | AAGCTAGCCCCCTGATAAAG | AAGCTAGCCCCCTGATAAAG |
| rs3055191 | CCCTCCTCTCTTCTCTACTG | CCCTCCTCTCTTCTCTACTG |
| rs6632943 | TTGACAAAGGAACACTGAGG | TTGACAAAGGAACACTGAGG |
| rs5924580 | CCGCAAACTATTGCAAGGAC | CCGCAAACTATTGCAAGGAC |
| rs4828544 | TATGAGGCATCAAGAGTAGC | TATGAGGCATCAAGAGTAGC |
| rs1807412 | AGTTCGAAACTAACCTGGCC | AGTTCGAAACTAACCTGGCC |
| rs2010456 | GTGGCTCACTTACGCCTATA | GTGGCTCACTTACGCCTATA |
| rs5969758 | AGCTATACCTTGATGCCTGC | AGCTATACCTTGATGCCTGC |
| rs1965051 | TGGTTAAACAGCAGTGTCTG | TGGTTAAACAGCAGTGTCTG |
| rs9887218 | CCATGTTTCCAAAGACAGGG | CCATGTTTCCAAAGACAGGG |
| rs1426800 | GTGGGATTCACTTCCTAAGG | GTGGGATTCACTTCCTAAGG |
| rs5969759 | ACAACTGGCACCATTTTAGC | ACAACTGGCACCATTTTAGC |
| rs6527766 | ATTCCACCCGTCAGCATAAG | ATTCCACCCGTCAGCATAAG |
| rs1365529 | TTTGCATGCACCCTAAAGAG | TTTGCATGCACCCTAAAGAG |
| rs5924582 | ATGATCGTACCACTGAACCC | ATGATCGTACCACTGAACCC |
| rs2114458 | TAATGTGGAGAACTCACAGG | TAATGTGGAGAACTCACAGG |
| rs5924583 | GGAGTTCAAGACCAACCTAG | GGAGTTCAAGACCAACCTAG |
| rs5924545 | TACTTGAGTGGCAAGTCAGG | TACTTGAGTGGCAAGTCAGG |
| rs5969761 | CGTGTTTGTGTTTCAGTATT | CGTGTTTGTGTTTCAGTATT |
| rs6629201 | CACTCACTCTCTGACTTACC | CACTCACTCTCTGACTTACC |
| rs6629202 | CTCTGCCTTTTCTATGTTTC | CTCTGCCTTTTCTATGTTTC |
| rs5924584 | ACTATTGTGTCCCAACTGCC | ACTATTGTGTCCCAACTGCC |
| rs5924585 | GTATCCATCCCCTCAAGCAT | GTATCCATCCCCTCAAGCAT |
| rs933946 | CCTTTAGTTCCTACCTTTGC | CCTTTAGTTCCTACCTTTGC |
| AL | AATACCTGCAGGCAGGTAAG | AATACCTGCAGGCAGGTAAG |
| rs5969762 | TTGGGATTGGAGACACATGG | TTGGGATTGGAGACACATGG |
| rs713355 | ATGTGGTCTATCAGAGCTAG | ATGTGGTCTATCAGAGCTAG |
| rs1365530 | CCTCTTCTCAAAACAGTTCTG | CCTCTTCTCAAAACAGTTCTG |
| rs1961490 | TAAAAACCCTGAGGGAGTTC | TAAAAACCCTGAGGGAGTTC |
| rs6527769 | TCTGAACAAAGCTGCAGCTG | TCTGAACAAAGCTGCAGCTG |
| rs2006847 | CACTGGAATCTGATCGTAGC | CACTGGAATCTGATCGTAGC |
| rs2382813 | ACCTAATTCAGCAGAGCACC | ACCTAATTCAGCAGAGCACC |
| rs2033120 | TTCTAGGACAGCAAAGGAGG | TTCTAGGACAGCAAAGGAGG |
| rs2162394 | TTTTTGCTGGTAGGGATGGG | TTTTTGCTGGTAGGGATGGG |
| rs1346246 | GTTTATATGCAGCACCTGGG | GTTTATATGCAGCACCTGGG |
| rs1365527 | ATGGTCTGTTTGAGCCTATC | ATGGTCTGTTTGAGCCTATC |
| rs1426801 | TGGTAGAACTGCAGTGTAAG | TGGTAGAACTGCAGTGTAAG |
| rs5924546 | ACCTCTGAAAACCTTCTTCG | ACCTCTGAAAACCTTCTTCG |
| rs5924547 | CACAGGCATCTGAGGAAATG | CACAGGCATCTGAGGAAATG |
| rs1365528 | CCTTTCTCACCCTTGCAAAC | CCTTTCTCACCCTTGCAAAC |
| rs4828524 | CCTGGAATGCAGCTGAAATG | CCTGGAATGCAGCTGAAATG |
| rs5924588 | TTAAAGTGCACAATTCAGTG | TTAAAGTGCACAATTCAGTG |
| rs5969764 | GGCAACCACTAACCTATTTTG | GGCAACCACTAACCTATTTTG |
| rs5924589 | ATTTTTGCCATCATCTTCCC | ATTTTTGCCATCATCTTCCC |
| rs5924590 | GCTGACTAGACAGATCTGAG | GCTGACTAGACAGATCTGAG |
| rs5924591 | GCACAACTTTCTTCCCAATAG | GCACAACTTTCTTCCCAATAG |
| rs5924592 | TAGCCAGGATGGTCTCGATC | TAGCCAGGATGGTCTCGATC |
| rs5924548 | CAGGTATGTGTCCGTTGTTC | CAGGTATGTGTCCGTTGTTC |
| rs5924593 | ATTACAAGCGTGAGCCATCG | ATTACAAGCGTGAGCCATCG |
| rs4828547 | AAGATTCTTCAGATGTGGCC | AAGATTCTTCAGATGTGGCC |
| rs714070 | GACAGCCAAGAGTGAACTAG | GACAGCCAAGAGTGAACTAG |
| rs5969768 | GATATTTGAAAGCTGACAAC | GATATTTGAAAGCTGACAAC |
| rs4828526 | CAGCAAGGACCTATAGTCCC | CAGCAAGGACCTATAGTCCC |
| rs5969738 | GTCAATTTCGTTGATCTTTGC | GTCAATTTCGTTGATCTTTGC |
| rs5924595 | GACACTTATTGATAAAGAGC | GACACTTATTGATAAAGAGC |
| rs5924549 | GAAGTACTCAGCATTTTGCTC | GAAGTACTCAGCATTTTGCTC |
| rs4828528 | TCCTATGACCCTGCCACATC | TCCTATGACCCTGCCACATC |
| rs5969769 | TCTACGCTTAAACCCATCGC | TCTACGCTTAAACCCATCGC |
| rs4007744 | CCCAAGTAGCTGGGACTACA | CCCAAGTAGCTGGGACTACA |
| rs2891073 | GACAGCAGAGAAGAGAAATG | GACAGCAGAGAAGAGAAATG |
| rs5924599 | CTCTGAATCCTTGTGTCAGG | CTCTGAATCCTTGTGTCAGG |
| rs1549531 | TTAGGAAACACTGCCTGGTC | TTAGGAAACACTGCCTGGTC |
| rs5924601 | ACACAGCACCTACTACAGTG | ACACAGCACCTACTACAGTG |
| rs5901609 | TGAATGAGTAACACTCAGGC | TGAATGAGTAACACTCAGGC |
| rs5969739 | CCCTTCTGATAACTCCTTCG | CCCTTCTGATAACTCCTTCG |
| rs5924602 | ACTTCAAACTGCACAGTCAC | ACTTCAAACTGCACAGTCAC |
| rs1426798 | ATTCACAGAGGCTTAGCAGG | ATTCACAGAGGCTTAGCAGG |
| rs7891150 | GGCTCTTCTTTCAGAGAGAG | GGCTCITCTTTCAGAGAGAG |
| rs4828550 | ACTCCAGCCTGGGCAACAAA | ACTCCAGCCTGGGCAACAAA |
| rs5924603 | GTTGCTAACATCTCCCACAG | GTTGCTAACATCTCCCACAG |
| rs1807947 | AACCATAGGGTGAGTTTGGG | AACCATAGGGTGAGTTTGGG |
| rs954253 | ATGTTCGTCTCACAGTGGAC | ATGTTCGTCTCACAGTGGAC |
| rs2382814 | TCAGCAAGTCCTACCAGTTC | TCAGCAAGTCCTACCAGTTC |
| rs1896178 | AGGTTCACGCCATTCTCCTG | AGGTTCACGCCATTCTCCTG |
| rs1896177 | AGGTTCACGCCATTCTCCTG | AGGTTCACGCCATTCTCCTG |
| rs5924605 | AGAGTGAGACTCTGTCTCAG | AGAGTGAGACTCTGTCTCAG |
| rs5924606 | GAGCCCAAUAACTCCAGTG | GAGCCCAATTAACTCCAGTG |

TABLE 38

Term

| | | |
|---|---|---|
| dbSNP rs # | Extend Primer | Mix |

| | | |
|---|---|---|
| rs1904525 | TCCCAGCACTTTGGGAG | ACT |
| rs5924557 | TTCTATCCCCTTACTTCC | ACG |
| rs5924616 | GAAGACAGCAATTTCCT | CGT |
| rs5924617 | TAAGACGTGCCTATGCT | ACG |
| rs7883612 | TTTGTCTGTGTCCCCAC | ACG |
| rs5924618 | CTAGGACTACAGGTGCA | ACG |
| rs5924619 | AAGGACACGACAGAAAG | ACT |
| AJ | CCTCGTCAGACATGGCTCA | CGT |
| rs5901596 | GGTGGTGGTGTGGTGGTG | ACT |
| rs7878645 | GAGCCAAGATCACGCCACC | ACT |
| rs5924564 | CTCATTATTCCCCAAAGT | ACT |
| rs5924535 | GGGAATGGAGATAAATTCAA | ACT |
| AK | TTCTTGATTTCCAACTGATT | ACT |
| rs6629197 | ACGGGATGGCTGCCGGG | ACG |
| rs7060904 | GACAACCAAGATGAAAAAC | CGT |
| rs4828523 | AGATTAGCATATTGATAGATTGC | ACT |
| rs5924568 | AGGCAGGAGGATCACTT | ACT |
| rs5924569 | TAGCTGGGCATGTTGGT | ACT |
| rs1904528 | TCACATTGCTCTGCACTTTTG | ACG |
| rs5924570 | CACGAGTGTGTTTTTATCT | ACT |
| rs1904527 | ACTCCTTTTTACTGCTGGGTA | ACT |
| rs2052763 | GAGTGAGTCCCTGTGCA | ACG |
| rs1549529 | TTTGGGAGGCCAAGGTA | ACT |
| rs5924573 | AGCAAAATCTTGGCCGGGC | ACT |
| rs5901600 | GGATTACAATGTCTTTTTTTTTT | ACG |
| rs2288304 | TGAAAAGAGAACAGATTAGTC | ACT |
| rs4240072 | GATGACTGCCACCTGCT | ACT |
| rs3055191 | CACTGGTTTTTTGTTTGTTT | ACT |
| rs6632943 | GGGGGATTGGGTTGGAC | ACT |
| rs5924580 | GTTCTCACTCATAGGTGGGAA | ACG |
| rs4828544 | AAGAGTAGCCAAACTCATA | ACT |
| rs1807412 | CAACATGGTAAAACCCC | ACT |
| rs2010456 | AGGCGGAGGCGGGTGGA | ACG |
| rs5969758 | ATGCCTGCAGGGTACAC | ACG |
| rs1965051 | CAGCAGTGTCTGAGGATTCAA | ACT |
| rs9887218 | CTTGAAGCGTTACCGCCTG | ACT |
| rs1426800 | CTAAGGAAAGCTCATGAC | ACG |
| rs5969759 | TCTTTCCCTTGATCTCAGGC | ACT |
| rs6527766 | CCCAGCACTGCACAAGCTG | ACT |
| rs1365529 | TTTTGTTGGCTGAGCAGAGAT | ACT |
| rs5924582 | GAACCCCAGCCTGGGTGA | ACG |
| rs2114458 | CAGCCCTGACAGAAAGAGC | ACG |
| rs5924583 | GCCAACATGGTGAAACCCC | ACT |
| rs5924545 | GGACCACTGAACCTCTCATT | ACG |
| rs596976 | TTCAGTATTTAACCAAAAAGTTTAAA | ACG |
| rs6629201 | CTTCCAGTCCTGCAAGCTC | ACG |
| rs6629202 | CTTTCTTTCTTCCTTTCTTCCTT | ACG |
| rs5924584 | TATTCAGTACAGGAACATGCC | ACT |
| rs5924585 | CCTTTGAGTTACAAACAATCCA | ACT |
| rs933946 | GATGTTTTTAACAGACTACAGGA | ACT |
| AL | AGGTAAGGCCTCACCATCAA | ACG |
| rs5969762 | AAGGGAATTAATAGCTGGCAAA | ACT |
| rs713355 | GTTATATTTAACAAGCACAGAAAA | ACT |
| rs1365530 | ATTAATGCCCAACTGATAAAAATA | ACT |
| rs1961490 | AATGTGATTTGGGGCCCTCA | CGT |
| rs6527769 | CTGCAGCTGATTTAGGTCTCA | ACT |
| rs2006847 | GGAAATGACAGGAAGCTGAAC | ACT |
| rs2382813 | GCAGAGCACCATGCACAATG | ACT |
| rs2033120 | GCAGGTAGGAGCAGGTAATG | ACT |
| rs2162394 | GGGGTGAGCCCTGCTGGT | ACT |
| rs1346246 | GGCTAGTACAGAACACATCAA | CGT |
| rs1365527 | GAGCCTATCTTAGTGCCCCT | ACT |
| rs1426801 | GATAGGATAACGAGGATACCAT | ACT |
| rs5924546 | TTAGAGTGGCATTTATGAAGTTA | ACT |
| rs5924547 | CTGAGGAAATGACAGACAGC | ACT |
| rs1365528 | CTCTCCACCCCGACCCC | ACG |
| rs4828524 | GAATGCAGCTGAAATGACAGAA | ACT |
| rs5924588 | TUCAGAATATTTTCATCCCCC | ACG |
| rs5969764 | CTGCAGATTTTCCTTTTCTGAA | ACG |
| rs5924589 | CATCATCTTCCCTCCCCCA | ACT |
| rs5924590 | CTGAGAAAGCAGGGAGTAAC | CGT |
| rs5924591 | TCTACATTTTTACAGCGTAGCA | CGT |
| rs5924592 | GTCTCGATCTCCTGACCTC | ACT |
| rs5924548 | GCCTGGCGCGATGGCTCA | ACG |
| rs5924593 | TATGCCAACTCTTACAATGAGA | ACT |
| rs4828547 | AGATGTGGCCAGATTATTTTAG | ACT |
| rs714070 | GTATGCTTGGTGGCTTATTGA | ACT |
| rs5969768 | ACAGGTTAAGTATCCAAAATCC | ACT |
| rs4828526 | GGACCTATAGTCCCAGCTAC | ACG |
| rs5969738 | CAACTTTTCAACTATTTTCTCTG | ACG |
| rs5924595 | TGATAAAGAGCAAAATGCTGAG | ACG |
| rs5924549 | TCCTAGTAACTAATTCAAAAGAAA | ACT |
| rs4828528 | GGGAACTCAGAGGCTGGCA | ACT |
| rs5969769 | GCGCCATCAAAACAGAACAC | ACG |
| rs4007744 | GCTGGGACTACAGGCGCC | ACG |
| rs2891073 | GAAATGAGGATTCCATTGCTCA | ACT |
| rs5924599 | ATCCTTGTGTCAGGATTCTAC | ACT |
| rs1549531 | ACTGCCTGGTCTATCTCCC | ACG |
| rs5924601 | ACTACAGTGGGCCTCCCC | ACT |
| rs5901609 | ACACTCAGGCCTGAGAATAC | ACG |
| rs5969739 | TAACTCCTTCGACCACAGTC | ACG |
| rs5924602 | AAACTGCACAGTCACCAGAAA | ACT |
| rs1426798 | CTTAGCAGGTGGATGGGGT | ACT |
| rs7891150 | CTTTCAGAGAGAGTGGGCC | ACG |
| rs4828550 | GGGCAACAAAGCGAGACTC | ACG |
| rs5924603 | CACAGAACCTGCTGCTTTTC | ACT |
| rs1807947 | GGTCCACTGTGAGACGAAC | ACT |
| rs954253 | GGACTCCCAAACTCACCCT | ACT |
| rs2382814 | CTACCAGTTCTAGATTCAAGATT | ACT |
| rs1896178 | AGTAGCTGGGACTACAGGC | ACT |
| rs1896177 | CTCCTGCCTCAGCCTCCC | ACT |
| rs5924605 | AACCCTCAATGCCATCCCC | ACT |
| rs5924606 | TAAGCTCAAATTGCAGAAGCC | ACT |

Genetic Analysis

Allelotyping results are shown for female (F) and male (M) cases and controls in Table 39 and Table 40, respectively. Allele frequency is noted in the fourth and fifth columns for melanoma pools and control pools, respectively.

TABLE 39
Females

| | | | | | | | | Melanoma |
|---|---|---|---|---|---|---|---|---|
| | Chromo- | Chromosome | | Female | Female | F | Odds | Associated |
| dbSNP rs# | some | Position | Alleles | Case AF | Control AF | p-Value | Ratio | Allele |
| rs1904525 | X | 16315373 | T/C | T = 0.523 | T = 0.575 | 0.129 | 1.233 | C |
| | | | | C = 0.476 | C = 0.424 | | | |
| rs5924557 | X | 16315940 | C/T | C = 0.654 | C = 0.598 | 0.087 | 0.788 | C |
| | | | | T = 0.345 | T = 0.401 | | | |
| rs5924616 | X | 16315984 | A/T | A = 0.562 | A = 0.505 | 0.095 | 0.797 | A |
| | | | | T = 0.437 | T = 0.494 | | | |
| rs5924617 | X | 16316317 | G/A | G = 0.730 | G = 0.708 | 0.501 | 0.900 | G |
| | | | | A = 0.269 | A = 0.291 | | | |
| rs7883612 | X | 16316522 | G/A | G = 0.515 | G = 0.520 | 0.897 | 1.017 | A |
| | | | | A = 0.484 | A = 0.479 | | | |
| rs5924618 | X | 16317598 | C/T | C = 0.339 | C = 0.274 | 0.061 | 0.734 | C |
| | | | | T = 0.660 | T = 0.725 | | | |
| rs5924619 | X | 16320441 | T/C | T = 0.471 | T = 0.355 | 0.001 | 0.618 | T |
| | | | | C = 0.528 | C = 0.644 | | | |
| AJ | X | 16325687 | G/T | G = 0.059 | G = 0.030 | 0.123 | 0.505 | G |
| | | | | T = 0.940 | T = 0.969 | | | |
| rs5901596 | X | 16328645 | —/TGG | — = 0.351 | — = 0.350 | 0.979 | 0.996 | — |
| | | | | TGG = 0.648 | TGG = 0.649 | | | |
| rs7878645 | X | 16329011 | T/C | T = 0.400 | T = 0.481 | 0.054 | 1.395 | C |
| | | | | C = 0.599 | C = 0.518 | | | |
| rs5924564 | X | 16329449 | C/G | C = 0.363 | C = 0.260 | 0.002 | 0.619 | C |
| | | | | G = 0.636 | G = 0.739 | | | |
| rs5924535 | X | 16339112 | A/G | A = 0.687 | A = 0.645 | 0.207 | 0.826 | A |
| | | | | G = 0.312 | G = 0.354 | | | |
| AK | X | 16340366 | A/C | A = 0.744 | A = 0.684 | 0.050 | 0.743 | A |
| | | | | C = 0.255 | C = 0.315 | | | |
| rs6629197 | X | 16348454 | C/T | C = 0.739 | C = 0.758 | 0.525 | 1.102 | T |
| | | | | T = 0.260 | T = 0.241 | | | |
| rs7060904 | X | 16355981 | G/T | G = 0.478 | G = 0.523 | 0.189 | 1.194 | T |
| | | | | T = 0.521 | T = 0.476 | | | |
| rs4828523 | X | 16356574 | A/G | A = 0.858 | A = 0.906 | 0.040 | 1.592 | G |
| | | | | G = 0.141 | G = 0.093 | | | |
| rs5924568 | X | 16357048 | C/G | C = 0.825 | C = 0.873 | 0.057 | 1.453 | G |
| | | | | G = 0.174 | G = 0.126 | | | |
| rs5924569 | X | 16357134 | A/G | A = 0.377 | A = 0.266 | ˜0.001 | 0.599 | A |
| | | | | G = 0.622 | G = 0.733 | | | |
| rs1904528 | X | 16365163 | C/T | C = 0.254 | C = 0.200 | 0.054 | 0.736 | C |
| | | | | T = 0.745 | T = 0.799 | | | |
| rs5924570 | X | 16365782 | C/G | C = 0.390 | C = 0.390 | 0.994 | 1.001 | |
| | | | | G = 0.609 | G = 0.609 | | | |
| rs1904527 | X | 16369641 | A/G | A = 0.543 | A = 0.478 | 0.082 | 0.771 | A |
| | | | | G = 0.456 | G = 0.521 | | | |
| rs2052763 | X | 16370572 | G/A | G = 0.298 | G = 0.219 | 0.006 | 0.661 | G |
| | | | | A = 0.701 | A = 0.780 | | | |
| rs1549529 | X | 16377566 | A/G | A = 0.437 | A = 0.311 | ˜0.001 | 0.580 | A |
| | | | | G = 0.562 | G = 0.688 | | | |
| rs5924573 | X | 16379322 | A/G | A = 0.521 | A = 0.413 | 0.008 | 0.646 | A |
| | | | | G = 0.478 | G = 0.586 | | | |
| rs5901600 | X | 16384421 | —/T | — = 0.849 | — = 0.873 | 0.329 | 1.222 | T |
| | | | | — = 0.855 | — = 0.885 | | | |
| rs5901600 | X | 16384421 | —/T | T = 0.150 | T = 0.126 | 0.329 | 1.222 | T |
| | | | | T = 0.144 | T = 0.114 | | | |
| rs2288304 | X | 16385394 | T/C | T = 0.729 | T = 0.701 | 0.392 | 0.871 | T |
| | | | | C = 0.270 | C = 0.298 | | | |
| rs4240072 | X | 16386837 | A/G | A = 0.346 | A = 0.229 | ˜0.001 | 0.562 | A |
| | | | | G = 0.653 | G = 0.770 | | | |
| rs3055191 | X | 16388721 | —/ | — = 0.499 | — = 0.488 | 0.770 | 0.959 | — |
| | | | | = 0.500 | = 0.511 | | | |
| rs6632943 | X | 16393300 | T/C | T = 0.461 | T = 0.520 | 0.080 | 1.268 | C |
| | | | | C = 0.538 | C = 0.479 | | | |
| rs5924580 | X | 16395181 | G/A | G = 0.847 | G = 0.794 | 0.049 | 0.693 | G |
| | | | | A = 0.152 | A = 0.205 | | | |
| rs4828544 | X | 16398058 | T/C | T = 0.783 | T = 0.855 | 0.006 | 1.633 | C |
| | | | | C = 0.216 | C = 0.144 | | | |
| rs1807412 | X | 16400368 | A/G | A = 0.596 | A = 0.620 | 0.506 | 1.107 | G |
| | | | | G = 0.403 | G = 0.379 | | | |
| rs2010456 | X | 16400419 | G/A | G = 0.022 | G = 0.015 | 0.607 | 0.699 | G |
| | | | | A = 0.978 | A = 0.985 | | | |
| rs5969758 | X | 16402566 | G/A | G = 0.248 | G = 0.192 | 0.041 | 0.719 | G |
| | | | | A = 0.751 | A = 0.807 | | | |
| rs1965051 | X | 16404435 | A/G | A = 0.341 | A = 0.232 | ˜0.001 | 0.585 | A |
| | | | | G = 0.658 | G = 0.767 | | | |
| rs9887218 | X | 16404794 | T/C | T = 0.007 | T = 0.043 | 0.069 | 6.157 | C |
| | | | | C = 0.993 | C = 0.957 | | | |
| rs1426800 | X | 16407797 | G/A | G = 0.752 | G = 0.860 | ˜0.001 | 2.027 | A |
| | | | | A = 0.247 | A = 0.139 | | | |
| rs5969759 | X | 16417444 | A/G | A = 0.631 | A = 0.681 | 0.124 | 1.245 | G |
| | | | | G = 0.368 | G = 0.318 | | | |
| rs6527766 | X | 16418221 | T/C | T = 0.801 | T = 0.812 | 0.703 | 1.069 | C |
| | | | | C = 0.198 | C = 0.187 | | | |
| rs1365529 | X | 16421764 | T/C | T = 0.824 | T = 0.890 | 0.008 | 1.738 | C |
| | | | | C = 0.175 | C = 0.109 | | | |
| rs5924582 | X | 16422381 | G/A | G = 0.800 | G = 0.827 | 0.324 | 1.199 | A |
| | | | | A = 0.199 | A = 0.172 | | | |
| rs2114458 | X | 16424226 | G/A | G = 0.808 | G = 0.852 | 0.081 | 1.366 | A |
| | | | | A = 0.191 | A = 0.147 | | | |
| rs5924583 | X | 16424670 | T/C | T = 0.701 | T = 0.726 | 0.421 | 1.132 | C |
| | | | | C = 0.298 | C = 0.273 | | | |
| rs5924545 | X | 16426360 | G/A | G = 0.813 | G = 0.868 | 0.029 | 1.510 | A |
| | | | | A = 0.186 | A = 0.131 | | | |
| rs5969761 | X | 16429055 | C/T | C = 0.760 | C = 0.901 | ˜0.001 | 2.881 | T |
| | | | | T = 0.239 | T = 0.098 | | | |
| rs6629201 | X | 16429589 | C/T | C = 0.657 | C = 0.625 | 0.339 | 0.871 | C |
| | | | | T = 0.342 | T = 0.374 | | | |
| rs6629202 | X | 16429694 | C/T | C = 0.704 | C = 0.786 | 0.013 | 1.544 | T |
| | | | | T = 0.295 | T = 0.213 | | | |
| rs5924584 | X | 16430036 | A/G | A = 0.795 | A = 0.873 | 0.001 | 1.779 | G |
| | | | | G = 0.204 | G = 0.126 | | | |
| rs5924585 | X | 16430411 | A/G | A = 0.870 | A = 0.868 | 0.929 | 0.981 | A |
| | | | | G = 0.129 | G = 0.131 | | | |
| rs933946 | X | 16432114 | T/C | T = 0.835 | T = 0.866 | 0.202 | 1.278 | C |
| | | | | C = 0.164 | C = 0.133 | | | |
| AL | X | 16434518 | C/T | C = 0.820 | C = 0.799 | 0.422 | 0.870 | C |
| | | | | T = 0.179 | T = 0.200 | | | |
| rs5969762 | X | 16437465 | A/G | A = 0.563 | A = 0.577 | 0.731 | 1.055 | G |
| | | | | G = 0.436 | G = 0.422 | | | |
| rs713355 | X | 16441918 | T/C | T = 0.559 | T = 0.559 | 0.995 | 1.001 | |
| | | | | C = 0.440 | C = 0.440 | | | |
| rs1365530 | X | 16442441 | G/C | G = 0.798 | G = 0.870 | 0.007 | 1.699 | C |
| | | | | C = 0.201 | C = 0.129 | | | |
| rs1961490 | X | 16442515 | A/T | A = 0.768 | A = 0.722 | 0.121 | 0.783 | A |
| | | | | T = 0.231 | T = 0.277 | | | |
| rs6527769 | X | 16444016 | C/G | C = 0.388 | C = 0.290 | 0.004 | 0.647 | C |
| | | | | G = 0.611 | G = 0.709 | | | |
| rs2006847 | X | 16448321 | A/G | A = 0.555 | A = 0.556 | 0.981 | 1.003 | G |
| | | | | G = 0.444 | G = 0.443 | | | |
| rs2382813 | X | 16451525 | A/G | A = 0.876 | A = 0.923 | 0.035 | 1.693 | G |
| | | | | G = 0.123 | G = 0.076 | | | |
| rs2033120 | X | 16451681 | C/G | C = 0.790 | C = 0.864 | 0.008 | 1.695 | G |
| | | | | G = 0.209 | G = 0.135 | | | |
| rs2162394 | X | 16452045 | A/G | A = 0.310 | A = 0.221 | 0.002 | 0.632 | A |
| | | | | G = 0.689 | G = 0.778 | | | |
| rs1346246 | X | 16453578 | C/A | C = 0.400 | C = 0.303 | 0.002 | 0.652 | C |
| | | | | A = 0.599 | A = 0.696 | | | |
| rs1365527 | X | 16454202 | G/C | G = 0.817 | G = 0.870 | 0.040 | 1.495 | C |
| | | | | C = 0.182 | C = 0.129 | | | |
| rs1426801 | X | 16454405 | T/C | T = 0.432 | T = 0.477 | 0.321 | 1.200 | C |
| | | | | C = 0.567 | C = 0.522 | | | |
| rs5924546 | X | 16455761 | G/C | G = 0.366 | G = 0.419 | 0.106 | 1.252 | C |
| | | | | C = 0.633 | C = 0.580 | | | |
| rs5924547 | X | 16457294 | G/C | G = 0.562 | G = 0.541 | 0.519 | 0.917 | G |
| | | | | C = 0.437 | C = 0.458 | | | |
| rs1365528 | X | 16459880 | G/A | G = 0.221 | G = 0.180 | 0.133 | 0.775 | G |
| | | | | A = 0.778 | A = 0.819 | | | |
| rs4828524 | X | 16462614 | A/G | A = 0.296 | A = 0.205 | 0.001 | 0.614 | A |
| | | | | G = 0.703 | G = 0.794 | | | |
| rs5924588 | X | 16463585 | C/T | C = 0.676 | C = 0.788 | ˜0.001 | 1.784 | T |
| | | | | T = 0.323 | T = 0.211 | | | |
| rs5969764 | X | 16469155 | C/T | C = 0.784 | C = 0.861 | 0.003 | 1.720 | T |
| | | | | T = 0.215 | T = 0.138 | | | |
| rs5924589 | X | 16473218 | T/G | T = 0.340 | T = 0.258 | 0.012 | 0.678 | T |
| | | | | G = 0.659 | G = 0.741 | | | |
| rs5924590 | X | 16475436 | G/T | G = 0.809 | G = 0.869 | 0.016 | 1.572 | T |
| | | | | T = 0.190 | T = 0.130 | | | |
| rs5924591 | X | 16478928 | G/T | G = 0.844 | G = 0.887 | 0.064 | 1.460 | T |
| | | | | T = 0.155 | T = 0.112 | | | |
| rs5924592 | X | 16480847 | A/G | A = 0.423 | A = 0.422 | 0.980 | 0.997 | A |
| | | | | G = 0.576 | G = 0.577 | | | |
| rs5924548 | X | 16480891 | G/A | G = 0.748 | G = 0.698 | 0.104 | 0.778 | G |
| | | | | A = 0.251 | A = 0.301 | | | |
| rs5924593 | X | 16480935 | C/G | C = 0.723 | C = 0.782 | 0.051 | 1.380 | G |
| | | | | G = 0.276 | G = 0.217 | | | |
| rs4828547 | X | 16482661 | A/G | A = 0.807 | A = 0.859 | 0.041 | 1.457 | G |
| | | | | G = 0.192 | G = 0.140 | | | |
| rs714070 | X | 16483528 | A/G | A = 0.566 | A = 0.552 | 0.685 | 0.945 | A |
| | | | | G = 0.433 | G = 0.447 | | | |
| rs5969768 | X | 16490615 | T/C | T = 0.077 | T = 0.078 | 0.959 | 1.014 | C |
| | | | | C = 0.922 | C = 0.921 | | | |
| rs4828526 | X | 16491635 | G/A | G = 0.221 | G = 0.157 | 0.021 | 0.655 | G |
| | | | | A = 0.778 | A = 0.842 | | | |
| rs5969738 | X | 16492763 | G/A | G = 0.538 | G = 0.566 | 0.431 | 1.117 | A |
| | | | | A = 0.461 | A = 0.433 | | | |
| rs5924595 | X | 16493358 | G/A | G = 0.772 | G = 0.848 | 0.011 | 1.655 | A |
| | | | | A = 0.227 | A = 0.151 | | | |
| rs5924549 | X | 16493425 | A/G | A = 0.699 | A = 0.777 | 0.011 | 1.502 | G |
| | | | | G = 0.300 | G = 0.222 | | | |
| rs4828528 | X | 16494704 | T/C | T = 0.317 | T = 0.316 | 0.973 | 0.995 | T |
| | | | | C = 0.682 | C = 0.683 | | | |
| rs5969769 | X | 16495532 | G/A | G = 0.800 | G = 0.878 | 0.003 | 1.801 | A |
| | | | | A = 0.199 | A = 0.121 | | | |
| rs4007744 | X | 16497657 | C/T | C = 0.532 | C = 0.636 | 0.006 | 1.537 | T |
| | | | | T = 0.467 | T = 0.363 | | | |
| rs2891073 | X | 16499364 | T/C | T = 0.368 | T = 0.172 | ˜0.001 | 0.356 | T |
| | | | | C = 0.631 | C = 0.827 | | | |
| rs5924599 | X | 16501050 | C/G | C = 0.531 | C = 0.535 | 0.920 | 1.013 | G |
| | | | | G = 0.468 | G = 0.464 | | | |
| rs1549531 | X | 16506161 | G/A | G = 0.719 | G = 0.819 | ˜0.001 | 1.774 | A |
| | | | | A = 0.280 | A = 0.180 | | | |
| rs5924601 | X | 16508122 | A/G | A = 0.148 | A = 0.153 | 0.813 | 1.047 | G |
| | | | | G = 0.851 | G = 0.846 | | | |
| rs5901609 | X | 16510285 | C/— | C = 0.269 | C = 0.175 | 0.001 | 0.578 | C |
| | | | | — = 0.730 | — = 0.824 | | | |
| rs5969739 | X | 16511416 | C/T | C = 0.297 | C = 0.223 | 0.011 | 0.680 | C |
| | | | | T = 0.702 | T = 0.776 | | | |
| rs5924602 | X | 16512549 | T/G | T = 0.617 | T = 0.603 | 0.662 | 0.941 | T |
| | | | | G = 0.382 | G = 0.396 | | | |
| rs1426798 | X | 16513760 | A/C | A = 0.337 | A = 0.257 | 0.008 | 0.681 | A |
| | | | | C = 0.662 | C = 0.742 | | | |
| rs7891150 | X | 16514001 | C/T | C = 0.986 | C = 0.966 | 0.227 | 0.409 | C |
| | | | | T = 0.014 | T = 0.034 | | | |
| rs4828550 | X | 16514202 | G/A | G = 0.758 | G = 0.850 | ˜0.001 | 1.820 | A |
| | | | | A = 0.241 | A = 0.149 | | | |
| rs5924603 | X | 16515789 | T/C | T = 0.319 | T = 0.238 | 0.011 | 0.665 | T |
| | | | | C = 0.680 | C = 0.761 | | | |
| rs1807947 | X | 16524486 | A/G | A = 0.347 | A = 0.262 | 0.005 | 0.667 | A |
| | | | | G = 0.652 | G = 0.737 | | | |
| rs954253 | X | 16524529 | A/G | A = 0.701 | A = 0.685 | 0.601 | 0.927 | A |
| | | | | G = 0.298 | G = 0.314 | | | |
| rs2382814 | X | 16525161 | T/G | T = 0.327 | T = 0.224 | ˜0.001 | 0.595 | T |
| | | | | G = 0.672 | G = 0.775 | | | |
| rs1896178 | X | 16525530 | A/G | A = 0.553 | A = 0.520 | 0.324 | 0.873 | A |
| | | | | G = 0.446 | G = 0.479 | | | |
| rs1896177 | X | 16525550 | A/G | A = 0.499 | A = 0.503 | 0.917 | 1.014 | G |
| | | | | G = 0.500 | G = 0.496 | | | |
| rs5924605 | X | 16525704 | C/G | C = 0.805 | C = 0.839 | 0.224 | 1.262 | G |
| | | | | G = 0.194 | G = 0.160 | | | |
| rs5924606 | X | 16528221 | A/G | A = 0.443 | A = 0.353 | 0.008 | 0.688 | A |
| | | | | G = 0.556 | G = 0.646 | | | |

TABLE 40
Males

| | | | | | | | | Melanoma |
|---|---|---|---|---|---|---|---|---|
| | Chromo- | Chromosome | | Male | Male | F | Odds | Associated |
| dbSNP rs# | some | Position | Alleles | Case AF | Control AF | p-Value | Ratio | Allele |
| rs1904525 | X | 16315373 | T/C | T = 0.569 | T = 0.597 | 0.487 | 1.122 | C |
| | | | | C = 0.430 | C = 0.402 | | | |
| rs5924557 | X | 16315940 | C/T | C = 0.592 | C = 0.591 | 0.975 | 0.996 | C |
| | | | | T = 0.407 | T = 0.408 | | | |
| rs5924616 | X | 16315984 | A/T | A = 0.495 | A = 0.456 | 0.292 | 0.854 | A |
| | | | | T = 0.504 | T = 0.543 | | | |
| rs5924617 | X | 16316317 | G/A | G = | G = | | | |
| | | | | A = | A = | | | |
| rs7883612 | X | 16316522 | G/A | G = 0.559 | G = 0.529 | 0.371 | 0.884 | G |
| | | | | A = 0.440 | A = 0.470 | | | |
| rs5924618 | X | 16317598 | C/T | C = 0.386 | C = 0.369 | 0.609 | 0.930 | C |
| | | | | T = 0.613 | T = 0.630 | | | |
| rs5924619 | X | 16320441 | T/C | T = 0.437 | T = 0.369 | 0.047 | 0.753 | T |
| | | | | C = 0.562 | C = 0.630 | | | |
| AJ | X | 16325687 | G/T | G = 0.038 | G = 0.015 | 0.185 | 0.385 | G |
| | | | | T = 0.962 | T = 0.985 | | | |
| rs5901596 | X | 16328645 | —/TGG | — = 0.351 | — = 0.340 | 0.728 | 0.950 | — |
| | | | | TGG = 0.648 | TGG = 0.659 | | | |
| rs7878645 | X | 16329011 | T/C | T = 0.452 | T = 0.473 | 0.575 | 1.086 | C |
| | | | | C = 0.547 | C = 0.526 | | | |
| rs5924564 | X | 16329449 | C/G | C = 0.325 | C = 0.258 | 0.042 | 0.722 | C |
| | | | | G = 0.674 | G = 0.741 | | | |
| rs5924535 | X | 16339112 | A/G | A = 0.634 | A = 0.633 | 0.997 | 1.000 | A |
| | | | | G = 0.365 | G = 0.366 | | | |
| AK | X | 16340366 | A/C | A = 0.687 | A = 0.705 | 0.605 | 1.088 | C |
| | | | | C = 0.312 | C = 0.294 | | | |
| rs6629197 | X | 16348454 | C/T | C = 0.760 | C = 0.761 | 0.969 | 1.006 | T |
| | | | | T = 0.239 | T = 0.238 | | | |
| rs7060904 | X | 16355981 | G/T | G = 0.479 | G = 0.525 | 0.187 | 1.200 | T |
| | | | | T = 0.520 | T = 0.474 | | | |
| rs4828523 | X | 16356574 | A/G | A = 0.873 | A = 0.903 | 0.191 | 1.367 | G |
| | | | | G = 0.126 | G = 0.096 | | | |
| rs5924568 | X | 16357048 | C/G | C = 0.847 | C = 0.885 | 0.117 | 1.390 | G |
| | | | | G = 0.152 | G = 0.114 | | | |
| rs5924569 | X | 16357134 | A/G | A = 0.324 | A = 0.266 | 0.088 | 0.756 | A |
| | | | | G = 0.675 | G = 0.733 | | | |
| rs1904528 | X | 16365163 | C/T | C = 0.244 | C = 0.207 | 0.219 | 0.810 | C |
| | | | | T = 0.755 | T = 0.792 | | | |
| rs5924570 | X | 16365782 | C/G | C = 0.424 | C = 0.409 | 0.665 | 0.941 | C |
| | | | | G = 0.575 | G = 0.590 | | | |
| rs1904527 | X | 16369641 | A/G | A = 0.500 | A = 0.470 | 0.468 | 0.888 | A |
| | | | | G = 0.499 | G = 0.529 | | | |
| rs2052763 | X | 16370572 | G/A | G = 0.278 | G = 0.214 | 0.034 | 0.709 | G |
| | | | | A = 0.721 | A = 0.785 | | | |
| rs1549529 | X | 16377566 | A/G | A = 0.396 | A = 0.250 | ˜0.001 | 0.510 | A |
| | | | | G = 0.603 | G = 0.749 | | | |
| rs5924573 | X | 16379322 | A/G | A = 0.479 | A = 0.408 | 0.057 | 0.751 | A |
| | | | | G = 0.520 | G = 0.591 | | | |
| rs5901600 | X | 16384421 | —/T | — = 0.831 | — = 0.863 | 0.211 | 1.283 | T |
| | | | | — = 0.867 | — = 0.890 | | | |
| rs5901600 | X | 16384421 | —/T | T = 0.168 | T = 0.136 | 0.211 | 1.283 | T |
| | | | | T = 0.132 | T = 0.109 | | | |
| rs2288304 | X | 16385394 | T/C | T = 0.676 | T = 0.689 | 0.726 | 1.062 | C |
| | | | | C = 0.323 | C = 0.310 | | | |
| rs4240072 | X | 16386837 | A/G | A = 0.305 | A = 0.227 | 0.018 | 0.668 | A |
| | | | | G = 0.694 | G = 0.772 | | | |
| rs3055191 | X | 16388721 | —/ | — = 0.497 | — = 0.479 | 0.621 | 0.933 | — |
| | | | | = 0.502 | = 0.520 | | | |
| rs6632943 | X | 16393300 | T/C | T = 0.504 | T = 0.503 | 0.967 | 0.994 | T |
| | | | | C = 0.495 | C = 0.496 | | | |
| rs5924580 | X | 16395181 | G/A | G = 0.811 | G = 0.775 | 0.264 | 0.803 | G |
| | | | | A = 0.188 | A = 0.224 | | | |
| rs4828544 | X | 16398058 | T/C | T = 0.797 | T = 0.852 | 0.039 | 1.464 | C |
| | | | | C = 0.202 | C = 0.147 | | | |
| rs1807412 | X | 16400368 | A/G | A = 0.582 | A = 0.629 | 0.202 | 1.214 | G |
| | | | | G = 0.417 | G = 0.370 | | | |
| rs2010456 | X | 16400419 | G/A | G = 0.023 | G = 0.026 | 0.799 | 1.154 | A |
| | | | | A = 0.976 | A = 0.973 | | | |
| rs5969758 | X | 16402566 | G/A | G = 0.238 | G = 0.200 | 0.193 | 0.801 | G |
| | | | | A = 0.761 | A = 0.799 | | | |
| rs1965051 | X | 16404435 | A/G | A = 0.305 | A = 0.239 | 0.036 | 0.716 | A |
| | | | | G = 0.694 | G = 0.760 | | | |
| rs9887218 | X | 16404794 | T/C | T = 0.040 | T = 0.038 | 0.946 | 0.959 | T |
| | | | | C = 0.959 | C = 0.961 | | | |
| rs1426800 | X | 16407797 | G/A | G = 0.798 | G = 0.871 | 0.005 | 1.711 | A |
| | | | | A = 0.201 | A = 0.128 | | | |
| rs5969759 | X | 16417444 | A/G | A = 0.621 | A = 0.672 | 0.165 | 1.252 | G |
| | | | | G = 0.378 | G = 0.327 | | | |
| rs6527766 | X | 16418221 | T/C | T = 0.787 | T = 0.799 | 0.704 | 1.073 | C |
| | | | | C = 0.212 | C = 0.200 | | | |
| rs1365529 | X | 16421764 | T/C | T = 0.836 | T = 0.874 | 0.131 | 1.359 | C |
| | | | | C = 0.163 | C = 0.125 | | | |
| rs5924582 | X | 16422381 | G/A | G = 0.780 | G = 0.797 | 0.542 | 1.109 | A |
| | | | | A = 0.219 | A = 0.202 | | | |
| rs2114458 | X | 16424226 | G/A | G = 0.814 | G = 0.838 | 0.381 | 1.186 | A |
| | | | | A = 0.185 | A = 0.161 | | | |
| rs5924583 | X | 16424670 | T/C | T = 0.715 | T = 0.726 | 0.711 | 1.059 | C |
| | | | | C = 0.284 | C = 0.273 | | | |
| rs5924545 | X | 16426360 | G/A | G = 0.822 | G = 0.860 | 0.141 | 1.333 | A |
| | | | | A = 0.177 | A = 0.139 | | | |
| rs5969761 | X | 16429055 | C/T | C = 0.811 | C = 0.899 | 0.001 | 2.076 | T |
| | | | | T = 0.188 | T = 0.100 | | | |
| rs6629201 | X | 16429589 | C/T | C = 0.609 | C = 0.632 | 0.486 | 1.103 | T |
| | | | | T = 0.390 | T = 0.367 | | | |
| rs6629202 | X | 16429694 | C/T | C = 0.733 | C = 0.785 | 0.151 | 1.328 | T |
| | | | | T = 0.266 | T = 0.214 | | | |
| rs5924584 | X | 16430036 | A/G | A = 0.826 | A = 0.878 | 0.039 | 1.517 | G |
| | | | | G = 0.173 | G = 0.121 | | | |
| rs5924585 | X | 16430411 | A/G | A = 0.851 | A = 0.851 | 0.997 | 0.999 | |
| | | | | G = 0.148 | G = 0.148 | | | |
| rs933946 | X | 16432114 | T/C | T = 0.834 | T = 0.855 | 0.434 | 1.173 | C |
| | | | | C = 0.165 | C = 0.144 | | | |
| AL | X | 16434518 | C/T | C = 0.765 | C = 0.779 | 0.673 | 1.078 | T |
| | | | | T = 0.234 | T = 0.220 | | | |
| rs5969762 | X | 16437465 | A/G | A = 0.599 | A = 0.564 | 0.308 | 0.866 | A |
| | | | | G = 0.400 | G = 0.435 | | | |
| rs713355 | X | 16441918 | T/C | T = 0.521 | T = 0.542 | 0.551 | 1.086 | C |
| | | | | C = 0.478 | C = 0.457 | | | |
| rs1365530 | X | 16442441 | G/C | G = 0.816 | G = 0.874 | 0.024 | 1.577 | C |
| | | | | C = 0.183 | C = 0.125 | | | |
| rs1961490 | X | 16442515 | A/T | A = 0.711 | A = 0.723 | 0.701 | 1.061 | T |
| | | | | T = 0.288 | T = 0.276 | | | |
| rs6527769 | X | 16444016 | C/G | C = 0.353 | C = 0.290 | 0.073 | 0.746 | C |
| | | | | G = 0.646 | G = 0.709 | | | |
| rs2006847 | X | 16448321 | A/G | A = 0.514 | A = 0.544 | 0.410 | 1.128 | G |
| | | | | G = 0.485 | G = 0.455 | | | |
| rs2382813 | X | 16451525 | A/G | A = 0.893 | A = 0.912 | 0.379 | 1.238 | G |
| | | | | G = 0.106 | G = 0.087 | | | |
| rs2033120 | X | 16451681 | C/G | C = 0.810 | C = 0.851 | 0.161 | 1.338 | G |
| | | | | G = 0.189 | G = 0.148 | | | |
| rs2162394 | X | 16452045 | A/G | A = 0.295 | A = 0.238 | 0.064 | 0.744 | A |
| | | | | G = 0.704 | G = 0.761 | | | |
| rs1346246 | X | 16453578 | C/A | C = 0.383 | C = 0.312 | 0.031 | 0.729 | C |
| | | | | A = 0.616 | A = 0.687 | | | |
| rs1365527 | X | 16454202 | G/C | G = 0.830 | G = 0.864 | 0.185 | 1.303 | C |
| | | | | C = 0.169 | C = 0.135 | | | |
| rs1426801 | X | 16454405 | T/C | T = 0.450 | T = 0.461 | 0.791 | 1.043 | C |
| | | | | C = 0.549 | C = 0.538 | | | |
| rs5924546 | X | 16455761 | G/C | G = 0.419 | G = 0.419 | 0.994 | 1.001 | |
| | | | | C = 0.580 | C = 0.580 | | | |
| rs5924547 | X | 16457294 | G/C | G = 0.523 | G = 0.551 | 0.437 | 1.115 | C |
| | | | | C = 0.476 | C = 0.448 | | | |
| rs1365528 | X | 16459880 | G/A | G = 0.206 | G = 0.180 | 0.381 | 0.848 | G |
| | | | | A = 0.793 | A = 0.819 | | | |
| rs4828524 | X | 16462614 | A/G | A = 0.271 | A = 0.211 | 0.049 | 0.719 | A |
| | | | | G = 0.728 | G = 0.788 | | | |
| rs5924588 | X | 16463585 | C/T | C = 0.713 | C = 0.785 | 0.026 | 1.469 | T |
| | | | | T = 0.286 | T = 0.214 | | | |
| rs5969764 | X | 16469155 | C/T | C = 0.817 | C = 0.877 | 0.066 | 1.605 | T |
| | | | | T = 0.182 | T = 0.122 | | | |
| rs5924589 | X | 16473218 | T/G | T = 0.319 | T = 0.267 | 0.104 | 0.778 | T |
| | | | | G = 0.680 | G = 0.732 | | | |
| rs5924590 | X | 16475436 | G/T | G = 0.831 | G = 0.871 | 0.121 | 1.369 | T |
| | | | | T = 0.168 | T = 0.128 | | | |
| rs5924591 | X | 16478928 | G/T | G = 0.850 | G = 0.885 | 0.166 | 1.361 | T |
| | | | | T = 0.149 | T = 0.114 | | | |
| rs5924592 | X | 16480847 | A/G | A = 0.416 | A = 0.413 | 0.938 | 0.989 | A |
| | | | | G = 0.583 | G = 0.586 | | | |
| rs5924548 | X | 16480891 | G/A | G = 0.697 | G = 0.707 | 0.774 | 1.045 | A |
| | | | | A = 0.302 | A = 0.292 | | | |
| rs5924593 | X | 16480935 | C/G | C = 0.716 | C = 0.815 | 0.010 | 1.744 | G |
| | | | | G = 0.283 | G = 0.184 | | | |
| rs4828547 | X | 16482661 | A/G | A = 0.810 | A = 0.847 | 0.162 | 1.300 | G |
| | | | | G = 0.189 | G = 0.152 | | | |
| rs714070 | X | 16483528 | A/G | A = 0.525 | A = 0.550 | 0.490 | 1.104 | G |
| | | | | G = 0.474 | G = 0.449 | | | |
| rs5969768 | X | 16490615 | T/C | T = 0.054 | T = 0.166 | ˜0.001 | 3.445 | C |
| | | | | C = 0.945 | C = 0.833 | | | |
| rs4828526 | X | 16491635 | G/A | G = 0.211 | G = 0.174 | 0.223 | 0.791 | G |
| | | | | A = 0.788 | A = 0.825 | | | |
| rs5969738 | X | 16492763 | G/A | G = 0.492 | G = 0.545 | 0.136 | 1.237 | A |
| | | | | A = 0.507 | A = 0.454 | | | |
| rs5924595 | X | 16493358 | G/A | G = 0.782 | G = 0.842 | 0.038 | 1.487 | A |
| | | | | A = 0.217 | A = 0.157 | | | |
| rs5924549 | X | 16493425 | A/G | A = 0.727 | A = 0.774 | 0.129 | 1.287 | G |
| | | | | G = 0.272 | G = 0.225 | | | |
| rs4828528 | X | 16494704 | T/C | T = 0.182 | T = 0.179 | 0.921 | 0.980 | T |
| | | | | C = 0.817 | C = 0.820 | | | |
| rs5969769 | X | 16495532 | G/A | G = 0.823 | G = 0.874 | 0.061 | 1.494 | A |
| | | | | A = 0.176 | A = 0.125 | | | |
| rs4007744 | X | 16497657 | C/T | C = 0.597 | C = 0.644 | 0.252 | 1.221 | T |
| | | | | T = 0.402 | T = 0.355 | | | |
| rs2891073 | X | 16499364 | T/C | T = 0.256 | T = 0.099 | ˜0.001 | 0.321 | T |
| | | | | C = 0.743 | C = 0.900 | | | |
| rs5924599 | X | 16501050 | C/G | C = 0.479 | C = 0.554 | 0.033 | 1.347 | G |
| | | | | G = 0.520 | G = 0.445 | | | |
| rs1549531 | X | 16506161 | G/A | G = 0.748 | G = 0.821 | 0.010 | 1.547 | A |
| | | | | A = 0.251 | A = 0.178 | | | |
| rs5924601 | X | 16508122 | A/G | A = 0.132 | A = 0.231 | ˜0.001 | 1.975 | G |
| | | | | G = 0.867 | G = 0.768 | | | |
| rs5901609 | X | 16510285 | C/— | C = 0.253 | C = 0.190 | 0.032 | 0.689 | C |
| | | | | — = 0.746 | — = 0.809 | | | |
| rs5969739 | X | 16511416 | C/T | C = 0.278 | C = 0.246 | 0.293 | 0.846 | C |
| | | | | T = 0.721 | T = 0.753 | | | |
| rs5924602 | X | 16512549 | T/G | T = 0.666 | T = 0.588 | 0.020 | 0.716 | T |
| | | | | G = 0.333 | G = 0.411 | | | |
| rs1426798 | X | 16513760 | A/C | A = 0.336 | A = 0.270 | 0.040 | 0.729 | A |
| | | | | C = 0.663 | C = 0.729 | | | |
| rs7891150 | X | 16514001 | C/T | C = 0.979 | C = 0.970 | 0.622 | 0.674 | C |
| | | | | T = 0.020 | T = 0.029 | | | |
| rs4828550 | X | 16514202 | G/A | G = 0.763 | G = 0.848 | 0.004 | 1.730 | A |
| | | | | A = 0.236 | A = 0.151 | | | |
| rs5924603 | X | 16515789 | T/C | T = 0.314 | T = 0.241 | 0.019 | 0.692 | T |
| | | | | C = 0.685 | C = 0.758 | | | |
| rs1807947 | X | 16524486 | A/G | A = 0.340 | A = 0.260 | 0.013 | 0.681 | A |
| | | | | G = 0.659 | G = 0.739 | | | |
| rs954253 | X | 16524529 | A/G | A = 0.659 | A = 0.681 | 0.514 | 1.101 | G |
| | | | | G = 0.340 | G = 0.318 | | | |
| rs2382814 | X | 16525161 | T/G | T = 0.293 | T = 0.231 | 0.059 | 0.726 | T |
| | | | | G = 0.706 | G = 0.768 | | | |
| rs1896178 | X | 16525530 | A/G | A = 0.541 | A = 0.511 | 0.404 | 0.887 | A |
| | | | | G = 0.458 | G = 0.488 | | | |
| rs1896177 | X | 16525550 | A/G | A = 0.498 | A = 0.501 | 0.931 | 1.012 | G |
| | | | | G = 0.501 | G = 0.498 | | | |
| rs5924605 | X | 16525704 | C/G | C = 0.802 | C = 0.839 | 0.214 | 1.289 | G |
| | | | | G = 0.197 | G = 0.160 | | | |
| rs5924606 | X | 16528221 | A/G | A = 0.419 | A = 0.340 | 0.025 | 0.713 | A |
| | | | | G = 0.580 | G = 0.659 | | | |

Allelotyping results were considered significant with a calculated p-value of less than or equal to 0.05 for allelotype results. These values are indicated in bold. The assay failed for those SNPs in which the allele frequency is blank. The combined allelotyping p-values for males and females were plotted in FIG. 6A and separately for females and males in FIGS. 6B and 6C, respectively. The position of each SNP on the chromosome is presented on the x-axis. The y-axis gives the negative logarithm (base 10) of the p-value comparing the estimated allele frequency in the case group to that of the control group. The minor allele frequency of the control group for each SNP designated by an X or other symbol on the graphs in FIGS. 6A, 6B and 6C can be determined by consulting Table 39 or 40. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brooks/Cole.). The black line (or generally top-most curve, e.g., see peak in left-most graph just to the left of position 92150000) provides a local test for excess statistical significance to identify regions of association. This was created by use of a 1 Okb sliding window with 1 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 10⁻⁸ were truncated at that value.

Finally, the exons and introns of the genes in the covered region are plotted below each graph at the appropriate chromosomal positions. The gene boundary is indicated by the broken horizontal line. The exon positions are shown as thick, unbroken bars. An arrow is place at the 3′ end of each gene to show the direction of transcription.

### Example 11

### Inhibition of NRP1 Gene Expression by Transfection of Specific siRNAs

RNAi-based gene inhibition was selected as an effective way to inhibit expression of NRP1 in cultured cells. Algorithms useful for designing siRNA molecules specific for the NRP1 targets are disclosed at the http address www.dhramacon.com. siRNAs were selected from this list for use in RNAi experiments following a filtering protocol that involved the removal of any siRNA with complementarity to common motifs or domains present in any target as well as siRNAs complementary to sequences containing SNPs. From this filtered set of siRNAs, four were selected that showed no off-target homology following BLAST analysis against various Genbank nucleotide databases. Table 41 summarize the features of the duplexes that were ordered from Dharmacon Research, Inc., and subsequently used as a cocktail in the assays described herein to inhibit expression of NRP1, NID2 ENDO180, CDK10, FPGT, CARK, PCLO or REPS2, respectively. A non-homologous siRNA reagent was used as a negative control.

TABLE 41

NRP1 siRNAs used for cell transfection

| | |
|---|---|
| siRNA | SEQ ID |

| | | | |
|---|---|---|---|
| siRNA | Target | Sequence Specificity | NO: |

| | | |
|---|---|---|
| NRP1_425 | NRP1 | AGAGAGGTCCTGAATGTTC |
| NRP1_1282 | NRP1 | GATTATCCTTGCTCTGGAA |
| NRP1_1910 | NRP1 | GCACCATACAATCAGAGTT |
| NRP1_2538 | NRP1 | GCCAGGCAATGTGTTGAAG |

Two melanoma cell lines (M14 and A375) and a breast cancer cell line (MCF7) were selected for RNAi experiments. On day 1, cells were transfected with siRNA cocktails (18.75 nM) using LIPOFECTAMINE 2000 (LF2000™) Reagent. On days 1, 3 and 6, cellular proliferation was measured using the WST-1 assay (Roche, catalog #1644807). Briefly, the WST-1 assay is a colorimetric assay used to determine cellular proliferation by measuring the cleavage of WST-1 by mitochondrial dehydrogenases in living cells. By measuring absorbance, a highly accurate measure of cellular proliferation is obtained. On day 1, WST-1 reagent was added to each well and allowed to incubate for 3-4 hours. Subsequently, absorbance was measured at 450 nm and 620 nm using a Tecan Ultra plate reader. This process was repeated on day's 3 and 6. The extent of proliferation on days 3 and 6 were calculated relative to day 1, based on absorbance readings for each sample on each day. From the triplicate repeats of each time point, means and standard deviations were calculated and the effect of siRNA inhibition of each target on cellular proliferation was assessed and compared to cells transfected with a positive control siRNA (siRAD21_{—}1175) and a negative control siRNA (siLuciferase GL2). All experiments were performed in duplicate.

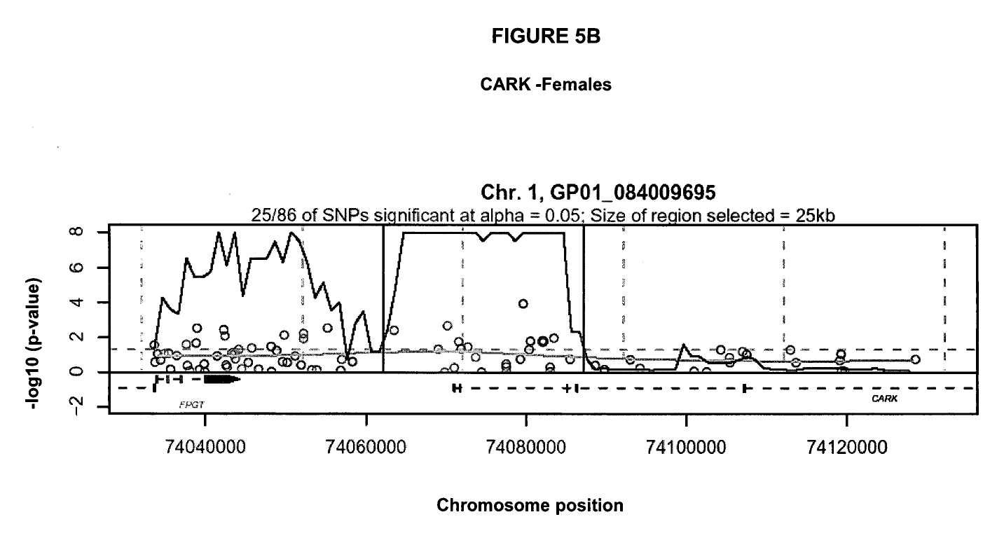

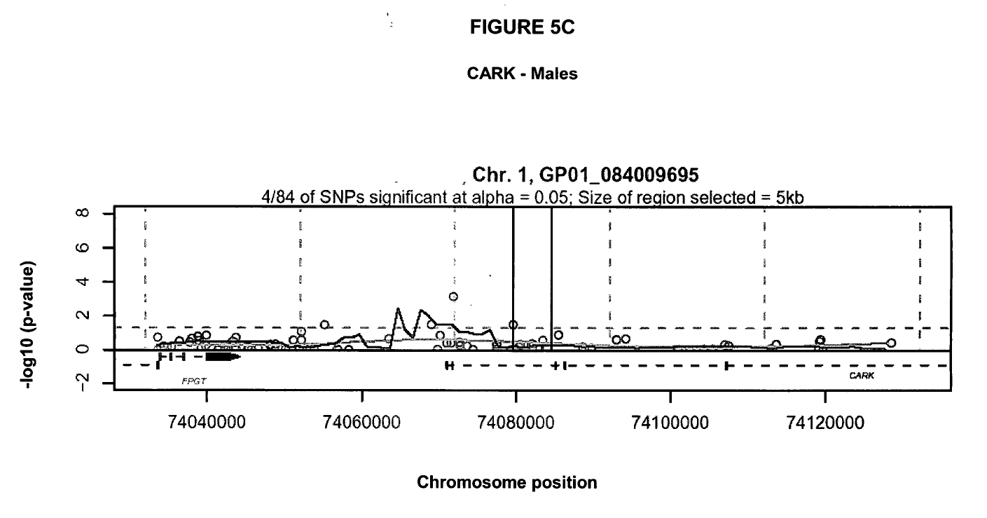

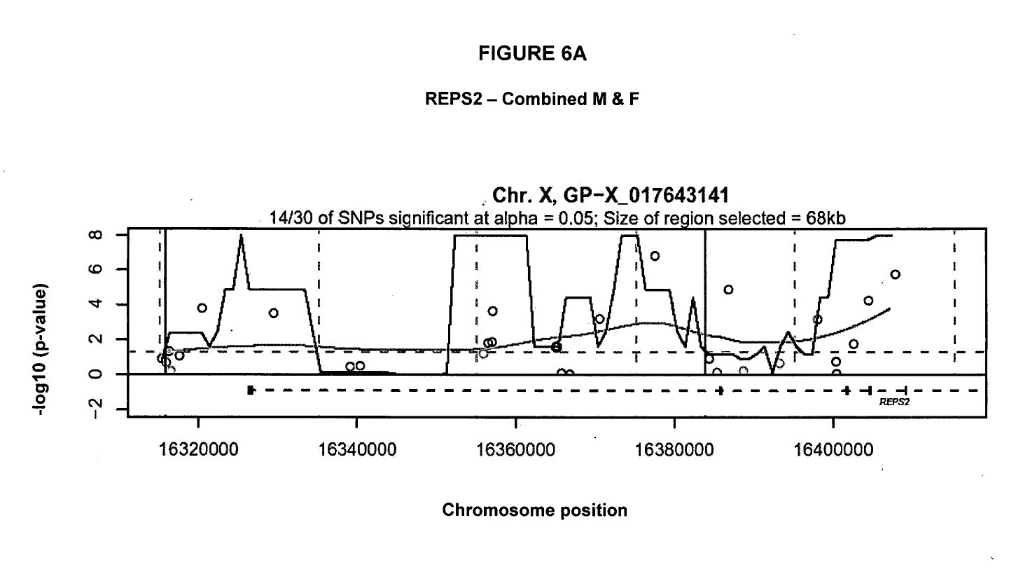

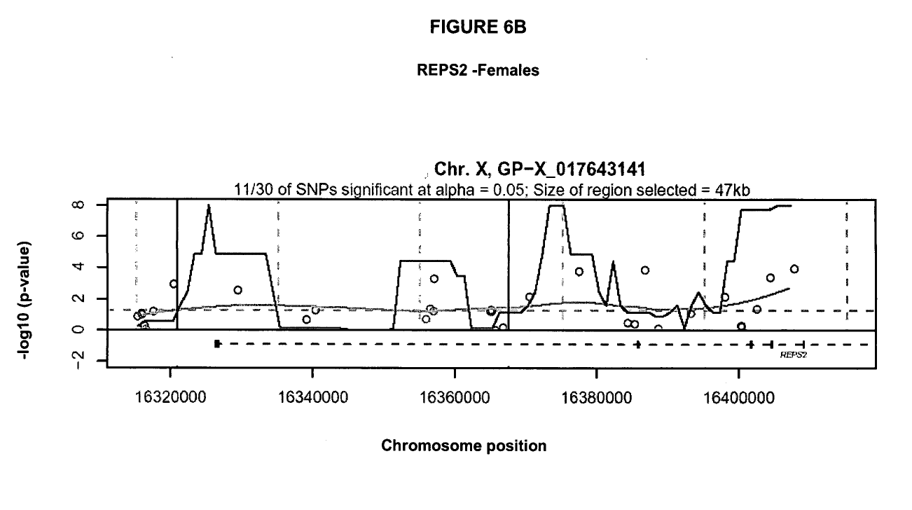

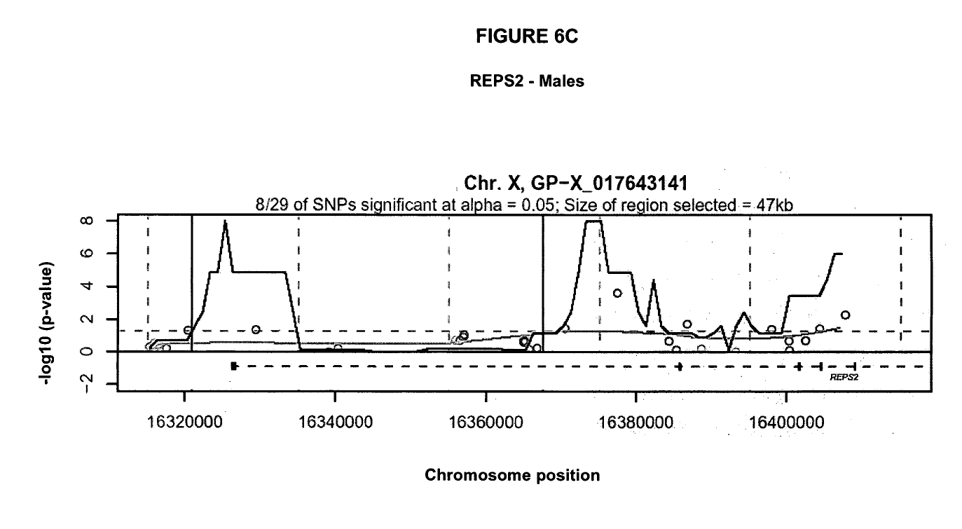

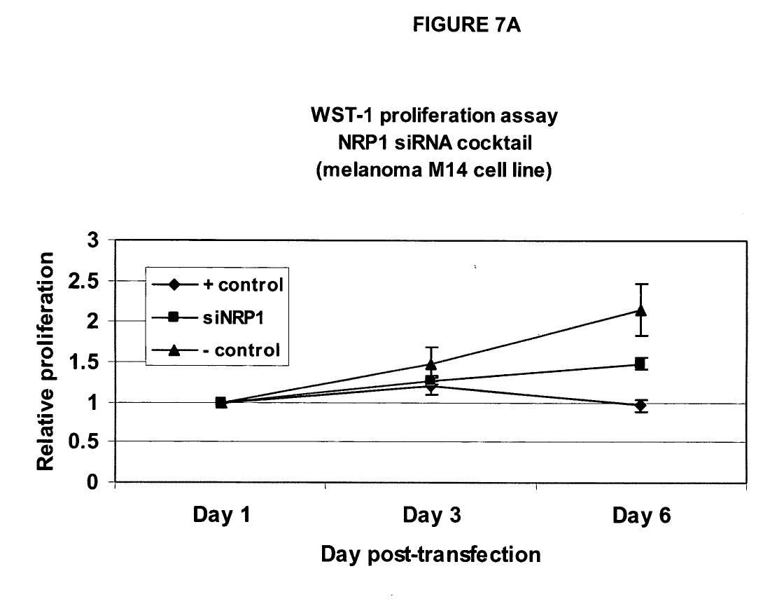

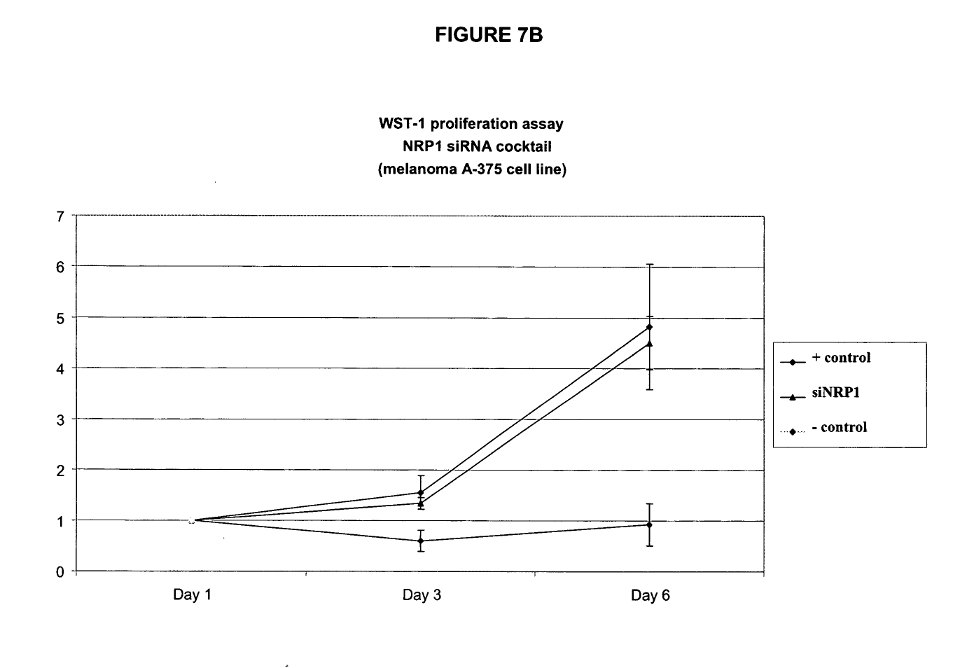

The difference in absorbance between these 2 wavelengths is an indication of the metabolic activity in each well that was measured. Metabolic activity is directly proportional to the number of cells in each well. Suppression of target mRNA levels correlated with decreased cell proliferation. The NRP1 siRNA suppressed proliferation of melanoma M14 cells and A375 cell (see FIGS. 7A and 7B respectively).

### Example 12

### Inhibition of CDK10 Gene Expression by Transfection of Specific siRNAs

RNAi-based gene inhibition was selected as an effective way to inhibit expression of CDK10 in cultured cells. Algorithms useful for designing siRNA molecules specific for the CDK10 targets are disclosed at the http address www.dhramacon.com. siRNAs were selected from this list for use in RNAi experiments following a filtering protocol that involved the removal of any siRNA with complementarity to common motifs or domains present in any target as well as siRNAs complementary to sequences containing SNPs. From this filtered set of siRNAs, four were selected that showed no off-target homology following BLAST analysis against various Genbank nucleotide databases. Table 42 summarizes the features of the duplexes that were ordered from Dharmacon Research, Inc., and subsequently used as a cocktail in the assays described herein to inhibit expression of CDK10. A non-homologous siRNA reagent was used as a negative control.

TABLE 42

CDK10 siRNAs used for cell transfection

siRNA

| | | | |
|---|---|---|---|
| siRNA | Target | Sequence Specificity | SEQ ID NO: |

| | | |
|---|---|---|
| CDK10_59 | CDK10 | GATCCGTCTGAAGTGTATT |
| CDK10_149 | CDK10 | GAAGCTGAACCGCATTGGA |
| CDK10_175 | CDK10 | CCTACGGCATTGTGTATCG |
| CDK10_532 | CDK10 | ACTTGCTCATGACCGACAA |

Two melanoma cell lines (M14 and A375) were selected for RNAi experiments. On day 1, cells were transfected with siRNA cocktails (18.75 nM) using LIPOFECTAMINE 2000 (LF2000™) Reagent. On days 1, 3 & 6, cellular proliferation was measured using the WST-1 assay (Roche, catalog #1 644 807). Briefly, the WST-1 assay is a colorimetric assay used to determine cellular proliferation by measuring the cleavage of WST-1 by mitochondrial dehydrogenases in living cells. By measuring absorbance, a highly accurate measure of cellular proliferation is obtained. On day 1, WST-1 reagent was added to each well and allowed to incubate for 3-4 h. Subsequently, absorbance was measured at 450 nm and 620 nm using a Tecan Ultra plate reader. This process was repeated on day's 3 and 6. The extent of proliferation on days 3 and 6 were calculated relative to day 1, based on absorbance readings for each sample on each day. From the triplicate repeats of each time point, means and standard deviations were calculated and the effect of siRNA inhibition of each target on cellular proliferation was assessed and compared to cells transfected with a positive control siRNA (siRAD21_{—}1175) and a negative control siRNA (siLuciferase GL2). All experiments were performed in duplicate.

### Example 13

### Cell Proliferation

The siRNAs from Example 12 were transfected in cell lines grown in 6-well plates. Cells were trypsinized on the following day and distributed into 96-well plates. Wst-1 reagent was added on the indicated days and the absorbance at 650 nm and 450 nm was measured. The difference in absorbance between these 2 wavelengths is an indication of the metabolic activity in each well that was measured. Metabolic activity is directly proportional to the number of cells in each well.

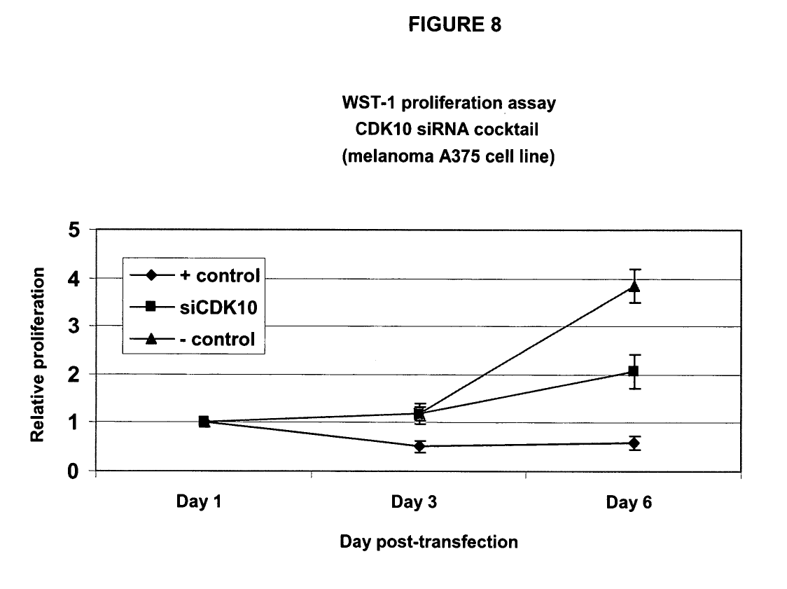

Suppression of target mRNA levels correlated with decreased cell proliferation as seen in A375 melanoma cells (see FIG. 8).

### Example 14

### Screening Assay to Detect Modulators of NRP1

The following is an exemplary assay for finding modulators of NRP1. Plasma membranes are prepared from cells expressing NRP1 by standard methods. The membranes are immobilized on wheat germ agglutinin-coated scintillation proximity beads (Amersham). Test samples and radiolabeled ligand, which is ¹²⁵I-semaphorin-3, are added, and bead-bound radioactivity is measured. The ability of a given sample to modulate binding activity is determined by comparison of bead-bound radioactivity in the presence of the compound with values obtained in solvent controls. The test compound optimally produces a result that differs by at least 3 standard deviations from a set of at least 30 replicate control samples.

### Example 15

### Screening Assay to Detect Modulators of NID2

The following is an exemplary assay for finding modulators of NID2. Plasma membranes as prepared are prepared from cells expressing integrin alpha-3-beta-1 or integrin alpha-6-beta-1 by standard methods. The membranes are immobilized on wheat germ agglutinin-coated scintillation proximity beads (Amersham). Test sample and radiolabeled ligand, which is ¹²⁵I-NID2, are added, and bead-bound radioactivity is measured. The ability of a given sample to modulate binding activity is determined by comparison of bead-bound radioactivity in the presence of the compound with values obtained in solvent controls. The test compound optimally produces a result that differs by at least 3 standard deviations from a set of at least 30 replicate control samples.

### Example 16

### Screening Assay to Detect Modulators of ENDO180

The following is an exemplary assay for finding modulators of ENDO180. Plasma membranes are prepared from cells expressing ENDO180 by standard methods. The membranes are immobilized on wheat germ agglutinin-coated scintillation proximity beads (Amersham). Test sample and radiolabeled ligand, which is ³H-mannose are added and bead-bound radioactivity is measured. The ability of a given sample to modulate binding activity is determined by comparison of bead-bound radioactivity in the presence of the compound with values obtained in solvent controls. The test compound optimally produces a result that differs by at least 3 standard deviations from a set of at least 30 replicate control samples.

### Example 17

### Screening Assay to Detect Modulators of CDK10

The following is an exemplary assay for finding modulators of CDK10. CDK10 may be screened using, for example, the non-radioactive IMAP system available from Molecular Devices, Sunnyvale Calif. 94089, according to the manufacturers instructions. The ability of a given compound to modulate activity of the kinase is determined by comparison of fluorescence polarization values in the IMAP assay in the presence of the compound with values obtained in solvent controls. Preferably, the test compound should produce a result that differs by at least 3 standard deviations from a set of at least 30 replicate control samples. More specifically, IMAP is a technology that uses the specific binding of metal (M III) coordination complexes to phosphate groups at high salt concentration and a fluorescence polarization readout. In a microwell assay format, fluorescently labeled peptides are phosphorylated in a kinase reaction. After the reaction, proprietary IMAP nanoparticles derivatized with metal (M^{III}) coordination complexes are added to the assay to bind the phosphorylated peptides. The binding causes a change in the motion of the peptide, and results in an increase in the observed fluorescence polarization. This assay, unlike antibody-based homogeneous kinase assays, is applicable to a wide variety of tyrosine and serine/threonine kinases. IMAP technology is largely independent of the sequence of the substrate peptides. In addition, the technology is useful in assays of phosphodiesterases and phosphatases. In a typical reaction on a 384-well plate the following would be combined: 10 ul of the kinase in question, 5 ul of test compound, 5 ul of substrate, such as Myelin Basic Protein Fragment 4-14 (MDL Number MFCD00133371; CAS Number 126768-94-3) or a similarly-sized peptide, at a concentration of 30-1000 nM; and ATP at a concentration of 1-30 ul. The substrate may be labeled with a fluorophore such as fluorescein-5-isothiocyanate (FITC Isomer I). These reagents would be allowed to react at room temperature for one hour. Then 60 ul of IMAP binding reagent is added and the fluorescence polarization is read.

### Example 18

### Screening Assay to Detect Modulators of TNNI3K (CARK)

The following is an exemplary assay for finding modulators of TNNI3K (CARK). TNNI3K (CARK) may be screened using, for example, the non-radioactive IMAP system available from Molecular Devices, Sunnyvale Calif. 94089, according to the manufacturers instructions. The ability of a given compound to modulate activity of the kinase is determined by comparison of fluorescence polarization values in the IMAP assay in the presence of the compound with values obtained in solvent controls. Preferably, the test compound should produce a result that differs by at least 3 standard deviations from a set of at least 30 replicate control samples. More specifically, IMAP is a technology that uses the specific binding of metal (M^{III}) coordination complexes to phosphate groups at high salt concentration and a fluorescence polarization readout. In a microwell assay format, fluorescently labeled peptides are phosphorylated in a kinase reaction. After the reaction, proprietary IMAP nanoparticles derivatized with metal (M^{III}) coordination complexes are added to the assay to bind the phosphorylated peptides. The binding causes a change in the motion of the peptide, and results in an increase in the observed fluorescence polarization. This assay, unlike antibody-based homogeneous kinase assays, is applicable to a wide variety of tyrosine and serine/threonine kinases. IMAP technology is largely independent of the sequence of the substrate peptides. In addition, the technology is useful in assays of phosphodiesterases and phosphatases. In a typical reaction on a 384-well plate the following would be combined: 10 ul of the kinase in question, 5 ul of test compound, 5 ul of substrate, such as Myelin Basic Protein Fragment 4-14 (MDL Number MFCD00133371; CAS Number 126768-94-3) or a similarly-sized peptide, at a concentration of 30-1000 nM; and ATP at a concentration of 1-30 ul. The substrate may be labeled with a fluorophore such as fluorescein-5-isothiocyanate (FITC Isomer I). These reagents would be allowed to react at room temperature for one hour. Then 60 ul of IMAP binding reagent is added and the fluorescence polarization is read.

### Example 19

### Screening Assay to Detect Modulators of FPGT

The following is an exemplary assay for finding modulators of FPGT. Incubate enzyme plus test sample plus substrates (fucose-1-phosphate and alpha-33P GTP (Amersham)) under conditions described (Pastuszak, I., Ketchum, C., Hermanson, G., Sjoberg, E. J., Drake, R. and Elbein, A. D. GDP-L-fucose pyrophosphorylase. Purification, cDNA cloning, and properties of the enzyme J. Biol. Chem. 273 (46), 30165-30174 (1998)). Add to the reaction mixture streptavidin coated scintillation proximity beads (Amersham) precoated with biotinylated lotus lectin (Vector Labs, Burlingame Calif. 94010). Measure bead-associated radioactivity by scintillation counting. The ability of a given compound to modulate activity of the enzyme is determined by comparison of bead-bound radioactivity in the presence of the compound with values obtained in solvent controls. Preferably, the test compound should produce a result that differs by at least 3 standard deviations from a set of at least 30 replicate control samples.

### Example 20

### In Vitro Production of Target Polypeptides

Target polypeptides encoded by the polynucleotides described herein may be produced by the methods described hereafter. cDNA is cloned into a pIVEX 2.3-MCS vector (Roche Biochem) using a directional cloning method. A cDNA insert is prepared using PCR with forward and reverse primers having 5′ restriction site tags (in frame) and 5-6 additional nucleotides in addition to 3′ gene-specific portions, the latter of which is typically about twenty to about twenty-five base pairs in length. A Sal I restriction site is introduced by the forward primer and a Sma I restriction site is introduced by the reverse primer. The ends of PCR products are cut with the corresponding restriction enzymes (i.e., Sal I and Sma I) and the products are gel-purified. The pIVEX 2.3-MCS vector is linearized using the same restriction enzymes, and the fragment with the correct sized fragment is isolated by gel-purification. Purified PCR product is ligated into the linearized pIVEX 2.3-MCS vector and E. coli cells transformed for plasmid amplification. The newly constructed expression vector is verified by restriction mapping and used for protein production.

E. coli lysate is reconstituted with 0.25 ml of Reconstitution Buffer, the Reaction Mix is reconstituted with 0.8 ml of Reconstitution Buffer; the Feeding Mix is reconstituted with 10.5 ml of Reconstitution Buffer; and the Energy Mix is reconstituted with 0.6 ml of Reconstitution Buffer. 0.5 ml of the Energy Mix was added to the Feeding Mix to obtain the Feeding Solution. 0.75 ml of Reaction Mix, 50 μl of Energy Mix, and 10 μg of the template DNA is added to the E. coli lysate.

Using the reaction device (Roche Biochem), 1 ml of the Reaction Solution is loaded into the reaction compartment. The reaction device is turned upside-down and 10 ml of the Feeding Solution is loaded into the feeding compartment. All lids are closed and the reaction device is loaded into the RTS500 instrument. The instrument is run at 30° C. for 24 hours with a stir bar speed of 150 rpm. The pIVEX 2.3 MCS vector includes a nucleotide sequence that encodes six consecutive histidine amino acids on the C-terminal end of the target polypeptide for the purpose of protein purification. target polypeptide is purified by contacting the contents of reaction device with resin modified with Ni²⁺ ions. target polypeptide is eluted from the resin with a solution containing free Ni²⁺ ions.

### Example 21

### Cellular Production of Target Polypeptides

Nucleic acids are cloned into DNA plasmids having phage recombination cites and target polypeptides are expressed therefrom in a variety of host cells. Alpha phage genomic DNA contains short sequences known as attP sites, and E. coli genomic DNA contains unique, short sequences known as attB sites. These regions share homology, allowing for integration of phage DNA into E. coli via directional, site-specific recombination using the phage protein Int and the E. coli protein IHF. Integration produces two new att sites, L and R, which flank the inserted prophage DNA. Phage excision from E. coli genomic DNA can also be accomplished using these two proteins with the addition of a second phage protein, Xis. DNA vectors have been produced where the integration/excision process is modified to allow for the directional integration or excision of a target DNA fragment into a backbone vector in a rapid in vitro reaction (Gateway™ Technology (Invitrogen, Inc.)).

A first step is to transfer the nucleic acid insert into a shuttle vector that contains attL sites surrounding the negative selection gene, ccdB (e.g. pENTER vector, Invitrogen, Inc.). This transfer process is accomplished by digesting the nucleic acid from a DNA vector used for sequencing, and to ligate it into the multicloning site of the shuttle vector, which will place it between the two attL sites while removing the negative selection gene ccdB. A second method is to amplify the nucleic acid by the polymerase chain reaction (PCR) with primers containing attB sites. The amplified fragment then is integrated into the shuttle vector using Int and IHF. A third method is to utilize a topoisomerase-mediated process, in which the nucleic acid is amplified via PCR using gene-specific primers with the 5′ upstream primer containing an additional CACC sequence (e.g., TOPO® expression kit (Invitrogen, Inc.)). In conjunction with Topoisomerase I, the PCR amplified fragment can be cloned into the shuttle vector via the attL sites in the correct orientation.

Once the nucleic acid is transferred into the shuttle vector, it can be cloned into an expression vector having attR sites. Several vectors containing attR sites for expression of target polypeptide as a native polypeptide, N-fusion polypeptide, and C-fusion polypeptides are commercially available (e.g., pDEST (Invitrogen, Inc.)), and any vector can be converted into an expression vector for receiving a nucleic acid from the shuttle vector by introducing an insert having an attR site flanked by an antibiotic resistant gene for selection using the standard methods described above. Transfer of the nucleic acid from the shuttle vector is accomplished by directional recombination using Int, IHF, and Xis (LR clonase). Then the desired sequence can be transferred to an expression vector by carrying out a one hour incubation at room temperature with Int, IHF, and Xis, a ten minute incubation at 37° C. with proteinase K, transforming bacteria and allowing expression for one hour, and then plating on selective media. Generally, 90% cloning efficiency is achieved by this method. Examples of expression vectors are pDEST 14 bacterial expression vector with att7 promoter, pDEST 15 bacterial expression vector with a T7 promoter and a N-terminal GST tag, pDEST 17 bacterial vector with a T7 promoter and a N-terminal polyhistidine affinity tag, and pDEST 12.2 mammalian expression vector with a CMV promoter and neo resistance gene. These expression vectors or others like them are transformed or transfected into cells for expression of the target polypeptide or polypeptide variants. These expression vectors are often transfected, for example, into murine-transformed cell lines (e.g. adipocyte cell line 3T3-L1 (ATCC), human embryonic kidney cell line 293, and rat cardiomyocyte cell line H9C2).

Provided hereafter are NRP1, NID2, ENDO180, CDK10, FPGT, PCLO and REPS2 genomic sequences (SEQ ID NO: 1, 2, 3, 4, 5, 6 and 7, respectively). Polymorphic variants are designated in IUPAC format. The following nucleotide representations are used throughout the specification and figures: “A” or “a” is adenosine, adenine, or adenylic acid; “C” or “c” is cytidine, cytosine, or cytidylic acid; “G” or “g” is guanosine, guanine, or guanylic acid; “T” or “t” is thymidine, thymine, or thymidylic acid; and “I” or “i” is inosine, hypoxanthine, or inosinic acid. SNPs are designated by the following convention: “R” represents A or G, “M” represents A or C; “W” represents A or T; “Y” represents C or T; “S” represents C or G; “K” represents G or T; “V” represents A, C or G; “H” represents A, C, or T; “D” represents A, G, or T; “B” represents C, G, or T; and “N” represents A, G, C, or T.

Following is a NRP1 genomic nucleotide sequence (SEQ ID NO: 1).

| >10:33587901-33809500 | | | | | | |
|---|---|---|---|---|---|---|
| 1 | gcaatgggtc | tcaagcaaag | cattcttcaa | gtgccccaca | gttatcaaat | aatttaaaaa |
| 61 | tcttagattt | tcatttttgt | gattatcttc | taaaaatggc | agttataccc | attgtggagc |
| 121 | acctagacag | gtaccatata | aggagtactg | ccatgtcttt | ttgtagcaac | acatatgttt |
| 181 | gtgttgatga | ttgaatgact | gccaagtgcg | gtgataggaa | agcatgaatc | cctataYagc |
| 241 | caaggttttc | cagttgttca | aaaataaaac | agtggtggga | gaggtgagcc | atcacttggg |
| 301 | aacacaggag | taacatggat | ctgaactcga | aagaccccgc | atcctggcct | ttggcagcat |
| 361 | gttcaagtct | gtggacaatt | gataagcaac | agttcattcg | tggtcagtct | agacataaat |
| 421 | aggcagtgaa | gaactttcca | ttagaagatt | ttcttcccag | accaggtgca | gtggctcacg |
| 481 | cctgtaatct | cagcattttg | ggaggctgag | gtgggaggac | tgcttgagta | caggagttcc |
| 541 | agaccagcct | gggcaaaaga | gccagatcct | gtctctacaa | aacaattttt | aagttagtca |
| 601 | ggcatggtgg | tgcacacctg | tagtcccagc | tacttgggag | gctgaggtgt | gaggatcact |
| 661 | tgagcccagg | aggttgaggc | tgcagtgagc | catgatcata | tcactgcact | cagtgtgggc |
| 721 | aataaagtga | aacccagtct | caaaaaaaca | aaagattttt | ctccctgtat | tatattttgg |
| 781 | gggtacacca | ttagatcatt | tggcctgtta | gtagataaaa | caaattaaaa | gataaaaagc |
| 841 | aaatacataa | aaataaacag | caaaggcaat | aatgatggct | aaaagggctg | cctattatgc |
| 901 | taaaactcaa | ggaaattaag | ttaataacaa | gcaaagccta | aagtgcaagt | acgttctact |
| 961 | agtatattac | ccaataacag | gaattttgct | tctacaaaat | gccatcagtc | atcacaccaa |
| 1021 | attaatgttg | tcatctgagt | gtttgggttt | tgtagttatg | ttgggattag | tgtgtaaagt |
| 1081 | tgcttggact | catttgttgt | atgggaatac | taagggtaaa | aaaatgtgtg | agtgaatttt |
| 1141 | ttgtaggtat | ataataagat | tagaataaaa | gagtgatctt | caacaccaaa | gatccttgga |
| 1201 | aatgtttccc | tttaaaagag | ggtcatacat | tccttaagtt | ggggaaatac | tgtgttttct |
| 1261 | ttcagctcca | tcctcgggcc | tttcagtgag | gacaggaacc | cacggccaga | caggaaggcc |
| 1321 | tgtttgaagc | caggttgcct | ccctagtaag | tttgttctgg | agccttctgg | tctccttttc |
| 1381 | ttttagttac | tttaagtggt | tttaggaaaa | agtactcttt | tcatcttaat | cctgctcctt |
| 1441 | acacccaagc | cagcccagag | ccatctggaa | tcctcagaac | agacctattc | tttgaagaga |
| 1501 | agaaggacaa | gatgtttttg | gacccttgca | ggaggtctgg | atacagacgg | ggccctgggg |
| 1561 | agggttgatc | cccagcagta | actgctcagc | tggaggcatg | aggggtccct | tctgacctcc |
| 1621 | aagcttcaga | ggggatggga | aaaggtaatt | tggcagcttt | gtgttccaag | gcttcacccc |
| 1681 | tagattggtt | taggcaagag | tcagtgagtc | cctgggaagg | cactgggcca | gcagagccca |
| 1741 | gacatcaagg | ctagcatttc | agtcttaata | atccaattta | ggctgggcgt | ggttgctcat |
| 1801 | gcctgtaatc | ccagcaattt | gggaggctga | ggcagatgga | tcatctgagg | ccaggagttc |
| 1861 | aagaccagta | tggccaacat | ggcgaaaccc | cgcctctact | aaaaatacaa | aaattagccg |
| 1921 | agcatggtgg | tgcatgcctg | tagtcccagc | tacttgagag | gcttaagcag | gagaatcact |
| 1981 | tgaacccagg | aggcggagtt | tgcagtcagc | caagatcgtg | ccaaaagaaa | aaaaaaagaa |
| 2041 | taaaaaacca | atttaaacaa | agaaaaacct | ttcattgagc | tctttaaata | tcctaagaat |
| 2101 | tgattataaa | tcacaaattg | gaagccaaca | taagctgtaa | Ygcaaagcaa | gcactgaaca |
| 2161 | tctctctacc | tgatgccact | taagttatgg | taaatttagt | caaggctgtg | ggttggggtt |
| 2221 | gtgtgcctta | ttgcaggaag | cagcacactt | ctgagggata | aatattgcat | ttggaaccta |
| 2281 | tgagttggta | gtttttctcc | aaggatgttc | ttaaaagcca | tttgcaatga | atttgtcagt |
| 2341 | ttctttagat | ttggatggaa | tataacgatt | ctggaacatt | actggggcct | ctgaaaattg |
| 2401 | tatttcctac | cttatatctt | acgtggctaa | agtacatggc | tgggagcggt | ggctcacgcc |
| 2461 | tctcatccca | gcaatttggg | aagccaaggt | gggtggatca | cctgaagtca | ggagtccaag |
| 2521 | accagcctgg | ccaacatggt | gaaaccctgt | ccctactaaa | aatacaaaaa | ttagctgggt |
| 2581 | ttggtggcat | gtgcctgtag | tcccagtcta | cttggaaggc | tgaggcagga | gaattgcttg |
| 2641 | aacccaggag | gcagaggttg | cagtgagctg | agactgtgcc | actgcactcc | agtctgggag |
| 2701 | acagagcgag | actttgtctc | aaaaaaaaaa | aaaaaaaaaa | agtacatctt | gttccaagaa |
| 2761 | acctactttt | ccttcaaaca | ctttgaggtt | taaaggcagg | atttactacc | ctctgtcctg |
| 2821 | acggcagagg | gagaaggagg | agaggaagga | acagaggctg | tcaccccttg | atgattttct |
| 2881 | gggatcctac | agttagatgg | gggtatggtc | tcctgcccta | tgaaaaccag | attctaggat |
| 2941 | gctattctaa | gaaaaaaaaa | Ntcccctgga | atcatattat | cttctgaaaa | ctagtgccac |
| 3001 | attttcctgt | tgatactata | ctcatcaact | tcagttcaca | gtcaaaattc | agttatcctg |
| 3061 | ttaatcttgc | tcattttggc | aggcacgaaa | atacacatgt | ttgtatggag | ccaagcaagt |
| 3121 | ttttgagatc | ttgggatcct | ctggaaatat | tttttcagtt | gttgaaaagg | aacttgcaag |
| 3181 | aaagcatata | ttttcaacac | tcgtcccagg | aacagtgtgt | gaaagtgaca | ttaaacttat |
| 3241 | ttctacactg | ggtgactagt | gactccctgc | tgggtaatca | ggtagacctc | tgtcatctct |
| 3301 | gcttcactgg | ggtttactgc | cagggcgtag | agagtctcat | ctcatggatc | ggaagaatgg |
| 3361 | gctgtacaca | cagacagctc | tcaccctcct | gttctggcgc | tgggctattc | ttcacggcca |
| 3421 | ctctttacac | tgagaaacac | ctcatgaatt | ttcagcctgg | aagacgatgg | gcccatttaa |
| 3481 | tgttctaacc | atatagagtg | cctgaattcc | caagatctcc | aactgctttt | cacttactac |
| 3541 | cctggaaaga | accccagtag | catccgttat | gaagacaaga | actccgcaaa | tctcctcctt |
| 3601 | gctggctggg | gagcaggagg | tctcataatt | ctgtatctgg | ccaggcatcg | tccttggctg |
| 3661 | catgttggtt | ttggcatcag | aggcagctgc | ctctaagccc | ttgttctgcc | atttcaagtt |
| 3721 | atgtattctt | aggcagatct | tttcatcttt | ttatacttag | tttcctcctt | tgtaagatga |
| 3781 | gaaagatagt | atcttgcagt | gtacttgtgg | cgattaaaga | ttatatttac | acgcatacca |
| 3841 | ccaggcacac | cgtagacacc | acacgaattc | tagctgtcat | attccaggcc | taggctagaa |
| 3901 | tgttgtcaga | ataaaagttt | catattcttc | ccactaatca | aaatactctc | accaccacca |
| 3961 | ctactgctat | tactacttct | acctaccatg | cattgagcac | ttatttgtgc | tgggaacttc |
| 4021 | actaggtact | ttgctgcata | agcttggttt | gttttatact | ttaaggaccc | cacaaggtag |
| 4081 | gcactatttt | tagccccact | tggtcactga | gataaatgag | gcatgatgag | gttacattgc |
| 4141 | tcaaggtcac | agaggaagtg | tctaaccctg | gctctgtctg | acccaaagtt | actgatctga |
| 4201 | aaacgatgct | acgccttttc | tagaaaatga | gccaaacctg | caccccaccc | tacaaagcag |
| 4261 | tccctgcccc | cctgaccctg | ccccaatcct | tagaactacc | tatcttcttt | gtattcatag |
| 4321 | ctcttccaag | caggagtcca | tcagcactgc | aggctgagca | caacatacca | gcttttgggt |
| 4381 | cagacaccag | ctgctgaaac | aggcccttca | atctgttgta | ctccccagaa | ttgcctctga |
| 4441 | tccttgcctg | acaaccatcc | caggatttca | gatcaagatg | tctgaggcag | gcccacttct |
| 4501 | aggtattttt | tgaaaaaatt | tgttacaggt | gacagggcgt | ggtggctcac | acctgtaatg |
| 4561 | ccagcacttt | gagaggctga | ggtgggagga | ttgcttgcgg | ccaggaggtc | aagaccagcc |
| 4621 | tgggcaacat | agtgagaccc | cagtctctac | aaacaaataa | aatttaaaaa | tcggctggtt |
| 4681 | ttagtggcac | gcacttgtgg | tcccagctac | tgaagagtct | gaagcaggag | gctcccttga |
| 4741 | gcccaggagt | ttgaggctgc | agtgagctat | gattgcatca | ctgctctcca | gactgcatga |
| 4801 | cagagagaga | ccccgtctct | aaaaaaaata | aaccaattgc | tacaggtaat | tgattctgat |
| 4861 | gacgatgctt | gcttgagaat | cctggagaga | tcctcgctgc | cttgatagaa | gctggggact |
| 4921 | ttagggggac | cctgaactcc | atctgaactc | ttggttattg | actgctctct | acttctctaa |
| 4981 | agagacggct | gtaaaaaatt | gacttagaaa | gccactcgaa | aaagttcaca | ctctgaactg |
| 5041 | cctgctaact | cccatttcct | accaaatcaa | gatgaacact | ttgcttgact | ttcacaatgt |
| 5101 | ctagcatctt | cacagtcccc | tgtattcctg | tacaacctgc | tccttctctt | ttctttgcta |
| 5161 | tcacggggac | accctgtgta | catttaggcc | tagagttttc | ttctaacctt | ggtgtcaaag |
| 5221 | tgtttccaat | caccaacgag | ctttcaaggg | gacaagaaag | ttccttctct | cctattttca |
| 5281 | ttggtcttct | aacagcaata | aaatttctac | acacttcaag | agtgtccacc | tggaaggaga |
| 5341 | aacatagtga | aggatgcaaa | tgtccatcaa | caaagtgcat | ctgtgtcaga | aagaactcag |
| 5401 | gtacaaatct | gtcactgcat | caggtgggag | ctctaaggct | ccactttgga | cagcggccag |
| 5461 | gagaggattt | ccaaagagtt | cgagttcctc | ccgcccggcc | tggacagaat | caggcaggat |
| 5521 | tatctgacag | gaggatgctg | cagtggttcc | tgagtgtgtg | cacatctcaa | cccccaatgc |
| 5581 | tggtgggaaa | ggaaaataaa | tcttggggcg | cccaaatcac | taagctaaag | ggaaaggtga |
| 5641 | agctgggaac | tgctcagggc | aaacctgcct | cccattgtat | tcaaagtcac | ccctctgctc |
| 5701 | acagagataa | atgcttattt | gattgcctcc | tttggagagg | ctagtcagaa | actcaaaaga |
| 5761 | atgcaaccat | ttgtctctta | tctacctttg | acctggaagc | ccctcccctg | ctttgagttg |
| 5821 | tcccgccttt | acttcgagtt | ctcctgccct | ttccagactg | aaccaatgtt | catcttacat |
| 5881 | atgttgattg | atgtctcatg | tctccctaaa | atgtattaaa | gcaagctgtg | ccctgaccac |
| 5941 | cttgggcaca | tgtcatcagg | acctgtgtca | tgggcatgca | tcctcaacat | tggcgaaata |
| 6001 | aactttctaa | attaactgag | acctgtctca | gatttttggg | gttcatactg | gctgcacccc |
| 6061 | tttccatggg | gttgctgccc | cattctctcc | tccatgggag | ccgaacaaag | tccaaatcag |
| 6121 | gacgttgcca | cacaaaacaa | cacaaatgtt | gtattattca | aataggagag | aggctcggtg |
| 6181 | ctgtcctcat | ttgggttatg | cctgcagtcc | agaggagcag | agtttggtgc | atggggagaa |
| 6241 | ctgaatgtcg | agggcttgac | agcctgttcg | cttggctcct | ctgtcctgag | attacattca |
| 6301 | ggattttctc | tttggcctga | agttagggag | gtaggtggaa | caagggtgac | ctttggagtg |
| 6361 | gaattttcat | aaaataaata | aactggatga | cagcgtataa | aagttacaaa | cctctttaga |
| 6421 | atgccatatg | tgagcaactt | gtaaacagtc | cataaatatc | acctgcaggg | tggaagtcaa |
| 6481 | ggaggctgat | gcagaattct | gtaggaacac | attttctgca | acactgaacc | atctggtaac |
| 6541 | gacaatttat | tctctggttg | gcataggagt | acaatgataa | aacatgttac | tactgacttc |
| 6601 | tgtctggaaa | aagacgatca | cggtccactg | tgttgtgtgg | acatggggcc | agtgcgcgtg |
| 6661 | actctcagcc | actcatatgc | cctgtggtgg | tgtcaggaga | ggaattaaaa | aattaaaggg |
| 6721 | gtgttttttg | gacatcccta | ctccacaagc | aggatttcaa | cagcaaacaa | caccaagaaa |
| 6781 | aaaaagtttt | tggagaaaaa | ttgttccttt | cataataatg | ccactttttg | agcgattcgt |
| 6841 | tttttaaaaa | gttcacaaac | atctgtgtca | ttcttgggga | ataagtgtag | aaagaacata |
| 6901 | tggatagcaa | ctttaccctt | tataaacatt | gcttattttc | aatatcccaa | taattagttc |
| 6961 | tagaataatg | aattctgcaa | aggataacaa | ggtctttcct | aggttctatt | tcacatagag |
| 7021 | aatttataaa | aagaaagaaa | aagagagcaa | gagacagaga | gagagagaaa | ggagttttga |
| 7081 | ttttcctcca | aatagactac | ttagtacaga | gtctctgtat | aataaaaaaa | aatttccaat |
| 7141 | ttctccaaag | tgaagaataa | tgtaacaacg | gaaacgcaac | tgaaggagat | gggctaatgg |
| 7201 | ctttattata | acaaacacac | atacaaacta | gacgcatttt | aatgtcatta | tcccctaccc |
| 7261 | cacccaacac | caaccttcta | actgtgcttt | taatcaagaa | ttccttctta | tgttgttact |
| 7321 | tcctgccctt | ctgggttcct | atgatatgca | gtttccatcc | tggtctcatc | ctgtttggcc |
| 7381 | cctttgcgta | aaagctaagg | atggctggcc | cacagcctac | cagggctttc | aaagccccag |
| 7441 | aagtctgtaa | acaggaaaga | tttcctctga | ggttgtgggc | tcctccttga | tcaaaagatg |
| 7501 | gctattggat | aagaacttcc | cctaaattgc | cagccactct | tattttccag | atgagatcct |
| 7561 | ctgttggagg | gtacattact | aggtatctat | ggcagaggtg | acaggcaatt | tgccaggact |
| 7621 | ccatgaagtt | gcaggagaag | taagttgcag | ctgctctgct | gttaatgagg | aggtagggct |
| 7681 | tggagaaaca | caaagattcc | tctgagcaat | ggccctggca | ggggtcccct | cctggttagc |
| 7741 | agccagggag | ggtgagcgaa | gcaaacatca | gttcagaata | tgaaagaggg | acagaaggac |
| 7801 | tgcatgaaga | gccccagcaa | ccttgagggt | ctcagaggtt | ggaaataaga | ataatgactt |
| 7861 | ccaccaaaaa | agtttagttt | tatccccatg | gttggtaagt | ggcctgtttt | tgagaaatgg |
| 7921 | gaggatagtc | aatgacttcc | tagttggaga | catcattcca | tgtaaaagga | ctgcttaggt |
| 7981 | ctgtttttag | ggtcaacttt | agagtcacag | ctcataattt | caaagttggg | tacatctttg |
| 8041 | tccaaggcag | cgaatctttc | ccccgtgatt | tcattatgag | ctaaccagtt | caggcaaaaa |
| 8101 | gctgtccctc | aagagtcact | caagaatttt | gcatttattt | ctagttaatc | acacctgtct |
| 8161 | ctgaaccttg | ccatggaaga | gtgaagtgag | ggagagaaca | aagcttttcc | tctaccttct |
| 8221 | ttctaaactg | ggtgagtaga | tcaaagtgta | ccaaaacaaa | caaacaaaca | ttttcgaaag |
| 8281 | accttctttc | ctgaatgcat | cttatcttgg | aacacatcct | tctttgttaa | ttacctgaca |
| 8341 | agaagaaagg | atgctttgaa | ttcttatcgt | agatttctct | gcaccactca | gagggcactt |
| 8401 | aataaattat | cttcttccat | ttactatctc | attatttgga | taaacttcat | cttttgaaat |
| 8461 | agattataaa | cttttcaatc | ccaggggatt | atagcgtagt | tcttcgcccc | ttccttccat |
| 8521 | actttttagg | tacgagcatc | ggtaaacagt | ttcgtattta | aactctactt | tatttcattt |
| 8581 | ctaattgaca | atgacctggg | ccttacttca | ttgtaataag | gaaacaaaat | gatgaagcca |
| 8641 | gaacttgcag | atggctgttc | tgtctgtttc | agtgtctcga | agtcagaggt | tggtggttaa |
| 8701 | tgataagaaa | gtaaatgtta | aatgtgcttt | tgcacattta | gcaaacagac | tgagaaccaa |
| 8761 | cttagaaaac | attttattta | caYtttagtt | atagcattca | ccttcttaag | atataacata |
| 8821 | tctgtttgca | agtcctgact | ccccacttga | ccgtgacccc | ttcaaggaca | ataaacaagt |
| 8881 | cattgtccat | ctttctatac | ccgtgtagta | ctcactatga | tactggcata | tgatatacac |
| 8941 | tgatataagc | tccagtgttt | tctctgatta | acatcaaata | atattgtcat | gacaatgaga |
| 9001 | aataagaaat | gtaggttctg | attttttccc | aaatcttgag | aatctgtttt | tccttcaagc |
| 9061 | tataaattgg | ggattcaacc | aaatcacatg | aagcagcaag | tatttcctgt | gtggcactta |
| 9121 | gctgttctgg | gaaataactc | aatgtcattt | ggttaacaca | cacacccagg | ccaaatagtt |
| 9181 | gaaaataacg | gaaagacatc | atggtaatta | ggttaggagt | tgacccaaga | catgttctca |
| 9241 | ttaaaattct | ttctatttct | ggtctccatt | ccgtcttctt | tcactgattg | acttttcttt |
| 9301 | ctttgaatga | aattccctcc | cccagatgga | ctatttcacc | acagcaaaaa | attgagaaag |
| 9361 | ctaagaagac | aatggggagg | aaaaccaata | ctaccaaaag | gaaacctcgg | agagttttgc |
| 9421 | tttctgtgag | ttctctgtca | gtcaaccctg | tcctgttcta | taagaccctt | ctcatagacc |
| 9481 | cctctttcaa | gggttttcaa | aaaggaaagc | ctatttattt | acttattacc | aattatgacc |
| 9541 | tcactggtct | tcccactctt | tagcactagg | gcaccagaac | cccagtgtat | gatgatggtc |
| 9601 | ccctagcagc | cattccaatg | acattattca | actcaaacag | agctttgcaa | taagctgtgt |
| 9661 | ctttccttat | gttctctggg | ggatgaaatg | agaatgaccc | catctaaaca | aaagaccaca |
| 9721 | tggttggcat | tcaacagtcg | actcctcccc | atttatttgt | ggatccaatt | cagtctaggt |
| 9781 | ctgaggcttt | acagaaatgc | atggcactcc | tcacagatgg | catctggcca | ccccttgaaa |
| 9841 | gcttctggtg | gaaggggagc | tcgctgcttt | tcgaggcagt | ctgtcacacg | agtagaaata |
| 9901 | gcgtagaaac | ccatatgtct | tgagtctaag | caatgacatt | caaaaagcag | aagcatccat |
| 9961 | gcaaccagat | tgacttaaag | taatgtaaac | agtccaatcg | attctttgaa | attgaagaat |
| 10021 | gacttgctgt | agagcattgt | gctccatgat | ggtgtgttct | gccagcctag | gcatgagcac |
| 10081 | tttgtggtag | tacaataaac | tactccaYag | cacatgggga | ggagaatgtg | aatcaatgat |
| 10141 | ttagtgtgat | ctcattggta | actcagtgct | gaagatctgc | tatcatatat | ttaggtgaat |
| 10201 | aactattcca | atgtgctgga | tggcacttta | caaacatata | taatctacaa | aaatgattta |
| 10261 | aaaaattttt | gtgcaccttt | caagtcaaaa | tagctcaaga | tataatgtgg | acatcctagg |
| 10321 | aaaataataa | ttgatgcatg | ttggaaacct | tttcatggta | tattagggct | ataatttcta |
| 10381 | cccattggtc | cctgttctgc | ccttaaggat | gacacagcat | aagttataaa | cccctgaagg |
| 10441 | tctagctgca | aaatgggtgc | atgcactccc | aaacaccaga | tcgtgattat | gcattacctg |
| 10501 | gaaaagtaag | aagtagggaa | agcaatatgg | ctgaagacaa | gtgtgttcag | tgaggggatc |
| 10561 | gttgtctcgg | gaatcctggg | aaccaagttc | tcactgtgga | cccactcact | gggcaagtca |
| 10621 | cttcctgcct | aaactccact | ttctctaccc | atcaactaga | gaggtcagat | tctccgttag |
| 10681 | ctgccaaaca | gtacatgggt | aaaattctat | aggtttaaaa | aatcatatac | catgactaaa |
| 10741 | agtctatgat | aacgggaaca | caattttggg | atctctttgt | aaaaaaaaaa | aaaaatctgt |
| 10801 | tctttttttc | ttttttcttt | gagatagagt | ctcactgtgt | tgcccaggct | ggagtgcaat |
| 10861 | ggcatgatct | cagctcactg | caacttccgc | ctcctgggtt | caagagattc | ttctgcctca |
| 10921 | gcctcccgag | tagctgggac | tacaggcaca | taccaccacg | cccggctaat | ttttgtattt |
| 10981 | ttagtagaga | tggggtttca | ccatgttggc | caggctggtc | tcgaactcct | cacctcgtga |
| 11041 | tctgcctgcc | tcagcctccc | aaagtgctgg | gattacaggc | gtgccaccgt | gccaggccag |
| 11101 | taaaaaatat | ttctaaccaa | aattttcact | ccagctgtca | tacatgtagg | gaagtgattg |
| 11161 | tttatattaa | tttgcaaagg | ctaaatccat | ttttcccccc | aactctttgt | gacttgcttg |
| 11221 | ggctagcctg | caaaaacact | tctgttcatg | gcagagagct | gtctgcatcc | agctgacatt |
| 11281 | gtttctctgg | cttaaataaa | agaggcagga | caatgagtgt | taaaccactg | ccggactctg |
| 11341 | atgtagtgtt | tacgaaggca | gctgcttttg | cattctgcgg | gtcagaaggg | tgtgtgtgtg |
| 11401 | tgtgtgtgtg | tgtgtgtgtg | cacgctcctg | catctatgta | agatctgaga | atcactgaaa |
| 11461 | gcagcttcct | tcatcctcta | ttttgacata | aagattctga | attcaaagca | ccttcagaag |
| 11521 | caacaagaga | ttaagacagg | ccaggcaacg | acttaatatg | gtaaaacata | tcttatccct |
| 11581 | gtggtcagtt | tacaatttcc | ttgacagata | actgctaatt | aagacatatt | atttcagaaa |
| 11641 | ttatggttNa | tttcctttgt | tatttacact | tttgttaaag | gtagtcaatg | atgtctttta |
| 11701 | gctgctcaat | aaagggtcac | ctaaggaaaa | gcaactgtat | gcctaacatc | tgggaaaaat |
| 11761 | attcccaatg | gggtaggttt | catgagaaca | aagttatttc | tcagatgact | ctttccttgc |
| 11821 | agcttcaagc | tttgttttcc | taggaattgt | gtagaaatgc | tggaattaat | gagtgataga |
| 11881 | gactttgatt | gaatcaatat | tgtgtgaaat | taacttgcaa | acatggttgt | gattactttt |
| 11941 | gtgggagttg | gactgaattt | ctcaagaaag | tacacaaaga | aaagactcag | ggcagagcta |
| 12001 | cagctgcctg | acagaggcaa | acagaagcct | aactttatgg | acttttccat | actctgcata |
| 12061 | tgttgaaatg | acaggctgat | tcatagcttc | cttgcttagt | ttccaaatcg | ctttgcaaca |
| 12121 | aaagcagatt | tactcccttc | ccaagactat | aactaggatt | catgatccca | taggctcaaa |
| 12181 | tcttcataaa | gggaattttg | caaagcatgc | ctcccaattc | ttgacagtca | caattcaaat |
| 12241 | attttcacaa | aacaacagga | ctctcaatta | agacagaggt | cagcaaacta | tggcctgtag |
| 12301 | atcaaaccta | tctgactgct | tgtttttgta | aataaagttt | tattggaaca | caaccatgct |
| 12361 | cattcatttg | cctattgtct | atggctactt | tcacagtatg | acggacgagc | tgaacagttg |
| 12421 | agattgaggc | tgtcttgcat | gcaaagccta | aaatatttcc | tatctggccg | ttttcagaaa |
| 12481 | gtgtgccaat | tcctgattta | aaatatcaat | ttgtgccata | aaaaaaaatc | aatttctttg |
| 12541 | aaaacataga | tatagtttta | aagtaaacct | ccccaacctc | ccctacctcc | atgaattatc |
| 12601 | ttctaaagca | aattggagta | tatatcaaga | gtcttaatac | tggttttatt | ctttagttca |
| 12661 | gcaaccacac | ttctagaaat | ctcttctaaa | gaaatcccta | agcatacatc | aagatttatg |
| 12721 | catacagatg | ttcattccat | cataatttat | aattatttat | aatcttgaaa | aattgaaaat |
| 12781 | gtcctaaata | ttctaggcca | tttagataaa | aattatttta | gaaagatgtg | atttaccaaa |
| 12841 | tgcttgtttt | taaaaatatt | taaagaatta | ccatttgacc | cagtaatctc | attactgggt |
| 12901 | atatacccaa | aggaatataa | atcattctac | cataaagaca | catgcacaca | tatgttcttt |
| 12961 | atagcactat | tagcaatagc | aaatacatgg | aatcaatcta | caagcccatc | aatggcagac |
| 13021 | tgaataaaga | aaatgtggtg | catgtacacc | acggaatact | acgcagccat | aaaaaagaat |
| 13081 | gagatcatgt | ccttttcagg | aacatggacg | cagctggagg | ccatcatcct | aaatgaacta |
| 13141 | gtgcacgaac | agaaaataaa | atagtgcatg | ttctcattta | taagtgggag | ctaaacattg |
| 13201 | aatacatatg | ggcgcaaaga | acaacagata | gcagggtcta | cttgaggatg | gagggtggga |
| 13261 | ggagggaaag | gatcaaaaaa | ctacctattg | tgtactgtgc | ttattacccg | agtgacaaaa |
| 13321 | taatctgtac | actaaaccct | tgtgacatgc | aatttaccaa | tataacaaac | ctgtacacgt |
| 13381 | acaggtttat | atataaaaca | aaagttttat | atatatacat | atatatgtat | ataatatata |
| 13441 | tatttaagag | cctaatataa | tgttttacat | gtaatttcaa | gagaaaatgg | taggataaaa |
| 13501 | gctgtctata | gaaaataatc | tcattattct | ttttttttga | gatggagtct | cactctgtcg |
| 13561 | cccacgctgg | agtttagtgg | cgcgatctcg | gctcactgca | gcctctgcct | cccgggttca |
| 13621 | agtgagtctc | ctgcctcagc | ctctggagta | gctgggatta | tgggcatgca | tcactatgtc |
| 13681 | cagctaattt | ttttgtactt | ttagtagaga | tggggtgtca | ctatgttggc | ctggctggtc |
| 13741 | ttgaactcct | gacctcaaat | gatctgcctg | acttggcctc | ccaaagtgct | gggattacag |
| 13801 | gtgtgagcca | ccatgcctgg | cctcaatttt | tttaaaaaat | gcatatttct | aaatgcaaag |
| 13861 | aaaataagca | gaaagaatat | atgccaaaaa | tatatatttt | aaaattcatt | tcagataatt |
| 13921 | ttgtgctcaa | gaaaaataaa | tatacatgcc | tttttgttta | atttatgaaa | atgttagtcc |
| 13981 | caaataaagt | tcttcttaat | ttatagaaat | gttagcccca | aataaagtta | gtacccattt |
| 14041 | aaaaatgagt | attaacctat | ttccaaaaca | gtccactttt | taatgtaata | aaaattaaat |
| 14101 | attttctaaa | atattatttt | tatgaatcac | attatctttt | catagatgga | gcagagatgg |
| 14161 | cagttttggg | gcacttgtgc | tgccactttt | tcctcttgcc | cacactgaac | actgcaacct |
| 14221 | attaagccat | ttctttctaa | gaagtttctc | aaatgtacta | cagatggcca | ctactaatca |
| 14281 | gtcagaagta | ttaagtgata | tcaaacatat | ttgccctctg | tgagagggct | acataagttt |
| 14341 | tccaagttaa | ttccaccctt | cctcttaaga | ctacatggtg | aaatacattt | ttatcaccgt |
| 14401 | agatcttgat | catcttgctt | cgaatgtctt | agaatgttct | gcgtgtattt | ctttcaaatc |
| 14461 | caaagaaatg | cgcggtgtct | cttcatatgc | attcaatgct | gaatgtctgt | gttaggatgt |
| 14521 | actgccccaa | gggctgggtg | aagctgctgc | ctcaagggtc | ctggtgagcc | tagttactgt |
| 14581 | atcgcccatg | tccacaattt | cctgtatttc | tttctagcac | ctgacactgt | gaacaatgct |
| 14641 | tccgtttttg | aaatgctcac | atcagcagct | ttctgtagca | agacactgtt | gtaactggta |
| 14701 | cctctctcat | aacgccttcc | cattgtcctt | ggaacaccct | ctaaccttaa | tccatcccat |
| 14761 | caagggggct | ttttctcgag | acatttattt | cttttctttt | tctccttgcc | cttctacgaa |
| 14821 | aagtcaattt | ctacagcttt | agccaacaag | tgattcccac | atttctatgt | ccagctttgc |
| 14881 | ccagtttccc | gtttctcaag | ttcttttttg | cacatctcta | cctgcaatgt | ttaaaagcct |
| 14941 | ctcaaaatgt | tcgaaatagg | acctactctc | ttgctgtatt | tgcctttttt | tttttttttg |
| 15001 | agacggagtc | tcgctctgtc | gcccaggctg | gagtgcagtg | gcgcgatctc | cgctcactgc |
| 15061 | aacctccgcc | tcccgggttc | acgccattct | cctgcctcag | cctcctgagt | atctgggact |
| 15121 | acagacgccc | Rccaccacgc | ccggctaatt | ttttttgtat | ttttagtaga | gacggggttt |
| 15181 | caccgtgtta | accaggatgg | tcttgatctc | ctgacctcat | gatctgcccg | cctcggcctc |
| 15241 | cgaaagtgct | gggattacag | gcgtgagcca | ctgcgcccgg | cctttttttt | tttttttttt |
| 15301 | tcgagacaga | gtcttgctct | gtcgcccagg | ctagtgtgca | atggcaccat | ctcggctcac |
| 15361 | tgcaacctcc | gcctcccagg | ttcaagcgat | tctcctgcct | cagcctcccg | agtagctggg |
| 15421 | attacaggtg | tgcaccacca | cgcctagcta | atttttgcat | ttttagtaga | gacggggttt |
| 15481 | taccatgatg | gccaggctga | ttttgaactc | ctgacctcaa | gtgattcact | cgacttggcc |
| 15541 | tcccaaagtg | ctaggattac | aggagtgagc | caccgcgccc | agctgcttta | tttactctta |
| 15601 | gtgttactag | taccactgtt | ctttcggacc | caaagatgga | aacatttgta | tcatcattaa |
| 15661 | catcctggcc | tccatctcct | ctcaaattct | atcactggtg | gagtcctttc | catgtttcct |
| 15721 | tcaggacgta | cctttctttc | taattttact | gccgccagtc | accttcagag | agtggctggg |
| 15781 | gttctttcgt | gggaagaaat | aagcgtgaaa | tggaaagaca | aagcattcta | ctaatgtatt |
| 15841 | tagtttttaa | catttattgg | ataattatta | tttttagacc | ccggcttggg | atgtggggaa |
| 15901 | gaaaagacga | cctacagttt | ctatttattg | tttctgttca | atttgtgcca | cttgaagagt |
| 15961 | cagtaaaatg | acgccaagac | cataaaccaa | ttccaaaaag | tcagagctga | cttggttcag |
| 16021 | tcccctttga | agagataagg | cctgtcattc | cagagtatga | ggcacagcag | gctggtgagt |
| 16081 | gtaaaggggt | tgattgggac | ctagaattat | aggcaaagaa | gacaaacatc | taagttagag |
| 16141 | ctagcagagg | gtgtctaaca | tggagaatac | gcatatgcta | gtcaagtgtc | cagggtcggg |
| 16201 | ggattgagtg | accttgggaa | acaagagtat | tccagctgat | ggctgaatct | cagagggtct |
| 16261 | aggaccccag | cagagggtta | caagtagaga | gcaccagcaa | ccaggaacaa | acctgatcct |
| 16321 | cagccaacaa | ttgcctctgc | cagatgtttt | ttatatgcgt | cttgtttaga | gcatgatcta |
| 16381 | ggtcccaggt | gggcctgcct | tgggggctgc | agatgaagat | aatgaggttc | tgcaagctgg |
| 16441 | aaaggcatgg | aggccactcg | gagaacagcc | cctgagtagc | aggtctcttc | tacttgtcag |
| 16501 | tgtctttcta | gagaataaat | atcacaaggg | cagagaatga | tgcctgtttt | gttcactgat |
| 16561 | ctatcactgt | ctctatcact | atcgctatca | gctgtcacag | tgatcggcac | atactggttg |
| 16621 | gtcaataaat | atttattgaa | tgaatgaatg | aatgtggtag | taagcaacaa | atgaagtttt |
| 16681 | cagaagaaga | attctggtgt | cccaagaaac | aatgcttggg | aaagtgcctg | aatctccctc |
| 16741 | aatttctgag | tttgtaagtt | ctgaatttat | taccacttat | atatattttt | aagcacatgt |
| 16801 | tgtgatgaat | gtttaaccat | gacctcatgt | tcaacatcaa | tctcaggact | tgtataatat |
| 16861 | tccacatata | aaccaaaccc | taaaacaaca | ttgataagaa | acaggaacag | tttaagtcgc |
| 16921 | aaggccagtc | tcaggaaagg | tgtaatgaca | aaaataatgg | gagttgacag | ttcaaatcct |
| 16981 | ttttttctca | taacagtgtt | tagaaatact | agacattttc | caatgaatgc | tgggaattag |
| 17041 | ctgaataact | ttagaagtta | gtcctctgtt | gagcacttgc | tttaacaaaa | atatggctca |
| 17101 | ggaggaccaa | atggaggaga | aaagtgcaat | taatatattc | actcatttta | ggtcctgttt |
| 17161 | acatttgcat | cttgaaggaa | ttgcatttat | tattttaatt | attttttaaa | tcaagtttcc |
| 17221 | tgtctgtaat | tgaatcatta | atagggctct | ttagtcttta | ttggtgacaa | acacatctat |
| 17281 | gtttaaattg | ctcagaattt | tctagtcaga | tgtgctaagg | acacattcag | aaagaaaaca |
| 17341 | agtttaaaac | acatctgtgt | ccttgtttag | cctttgaaaa | gctactttat | cattatacat |
| 17401 | tttgggcatt | tcctttaaaa | atgggtctgg | tacaactaaa | ccatttccca | aggatgtgtg |
| 17461 | aattaaccag | tggttacaaa | gagcttcgaa | atgatgatgg | actatcagtg | ctaagtcaaa |
| 17521 | ttatgaaatt | gcacctttgc | aaaaatgcta | ttgtaggaat | aaatgaatct | gaacgtggta |
| 17581 | tttacagtgt | gatttgcttc | tgaaatagaa | tatttctaat | gggcctctgc | aggcaacaac |
| 17641 | aaagtgccat | cttatctcct | gttgatagac | cacaatttca | gaaaattatt | tttacaatgg |
| 17701 | catctgaata | agctccttaa | tttgagactg | ttttccaaat | agatacatag | cttattactt |
| 17761 | tgcttacgct | gcctttgaga | cacaaagagt | gaggtgggat | tcattaacaa | aatcgctgtt |
| 17821 | gtcattcctt | tgtggttttt | ttttcaaagt | aatttctctt | ctttacaagc | taatgttaat |
| 17881 | gagctatgtt | gaataaaaag | atcaggtaaa | agctactttt | gtgttagtga | tcaaatccga |
| 17941 | gtttgggtta | atactcaggt | tatcaattgc | acttaaagca | cacctcaaac | ttggtttgcg |
| 18001 | gctcctgtag | tttgaagacc | aaaccaatca | ctcatgtatg | gaatttctac | tgactgtgaa |
| 18061 | tgttatattc | aacaacagca | gaacaataca | tgtgtatttc | agaagaccac | attctcagtt |
| 18121 | cttctaaata | actgattaga | taaaatgcat | ctgttcatgg | gtaagactaa | ggtctaggtg |
| 18181 | aaaaagtagg | caattcctta | gaataaattt | tgccaaaaca | aggtctaaaa | cgtctgatac |
| 18241 | cacttagggg | ttgaaaactg | acataaaaca | tgtgtctcag | tgaaatatga | gtaggaagga |
| 18301 | agagaaggct | gtgttgcaaa | agtcaaggag | ggtctgggct | gggctggaac | agcagcttgg |
| 18361 | atctagggaa | gaagaaagca | aaaagtaggg | agggtcttgc | aaagaccaaa | gaaagcccca |
| 18421 | gggatcagga | ccagcctcag | ctgtcagaaa | agacatctct | gattattaca | cattgtaggc |
| 18481 | ctgtatcaaa | atagctcatg | taccccagta | atgtataYat | ctgctacata | cccaaaaatt |
| 18541 | aaaagttaaa | aaaaaaagac | atctttgtaa | tatgctctat | ggcaggagga | ttttttctgg |
| 18601 | agaattagaa | tcctgttact | ttagagacaa | aagtgcttaa | agagaagagc | ttcattcctc |
| 18661 | tcatttcaca | gatgaagaga | caaaggccca | gaaagtctca | atacttgagc | tcacagctaa |
| 18721 | ttagagctca | agtgattcct | agaactcagg | atcacaatga | gtacctgcgg | gcatcacaca |
| 18781 | tgaagaccgc | accgttaact | gagcaaattt | cttgagagag | ctacggacat | ggtccacaca |
| 18841 | ggacgggcac | caaggagaca | aagctgaggg | cacaatcaag | agaagcttct | gcctctgccc |
| 18901 | cagacagtct | tttgtctttc | atcccatgga | aatggaaatg | caaacgttta | ttagtttatt |
| 18961 | acgtaagttc | cttccttcct | tccttccttc | cctccctctc | tccctccttt | ctctctttcc |
| 19021 | ctccctccct | cctttcRttt | tccttccttc | cttcYYtcYc | tctctYtctY | tctctctYtc |
| 19081 | tttctttcta | ttcctccctc | cctccctccc | tccctcctct | ctctctctct | attttcagag |
| 19141 | gtagactgtt | gcactgtggc | ccaggctaga | gtgcagtggt | gtgatcatgg | ctcactgcag |
| 19201 | ccttgaactc | ctggggttac | cgtgatcctt | cctccacagc | ctcccgagta | actaagacta |
| 19261 | cgggcgcatg | gcaccatgct | tggctaattt | tttaaaagtt | tttatagaga | cggtattgcc |
| 19321 | caggctgttc | tcaaactcct | ggcctcacgc | catcctccca | tcttggtctc | ccaaagtatt |
| 19381 | gggattacag | gcatgagcca | ctgtgtccag | cgtcatgtaa | atcattcttt | tctatggttg |
| 19441 | cagacaaact | agagaggtga | gaaggaggga | gtgctttctg | tttgtttcta | aaatagacag |
| 19501 | ataacagaaa | catttggaaa | tattgcggaa | aactaagtgt | aagagagtaa | cattgaagag |
| 19561 | ctagtaagaa | agataaaacc | agctgtggag | gctgggtttg | cctgttgtag | gtgtcatcat |
| 19621 | acatctacta | ctttgcctct | tcctcttggg | ctggcatgaa | cacattgtgc | cacacgcctt |
| 19681 | aagaaacact | taggaaacac | tggtatggag | ggagagggga | gagcccaggc | agcagtggaa |
| 19741 | tcctctcatt | gccccctcca | gaaacaagca | ggcctgggct | agttcctcca | agtagcatct |
| 19801 | ttgtctagga | caggaggctt | gttatttttc | tggtggtatg | gactgacata | tttacaccat |
| 19861 | tcttcaagga | acgcatccca | tgttaacttc | tctaatgaag | aaattcactt | tctgcctctt |
| 19921 | ccctggcaag | cctgctgtga | agttaacatg | tgcatttact | ggggttgcca | gctggaacgg |
| 19981 | aagcaggctc | taaattattt | gtgtttaatt | tatgtaaagg | tggtttggac | ctgagcccag |
| 20041 | atgtgaatat | acaatagacc | aagtttacag | aattgtgaca | caaagtccca | agagttgtgg |
| 20101 | ggatgtcaga | aaacactcag | caatcaggac | gtatgcctac | aacattaata | cattttatgc |
| 20161 | gttcaactcc | aaaacagatt | atggagacga | ctgtaagaca | gaaagaaaaa | ctttcagcca |
| 20221 | agcactatct | ctgcagattt | tttgagatgt | agctagagct | actcaaatgt | aaaaattcaa |
| 20281 | gcttttaaaa | atagaggttt | ttagggggaa | tatgtaagtc | tgactcacta | cccaggaaaa |
| 20341 | tgaagggttt | accatgtaaa | tgagaagaat | aagtgagcat | tttacagtat | gattaagtta |
| 20401 | agcctttgtc | cagagagctg | acttggtctt | cctgtgctgg | aggaacacac | tggagaagtc |
| 20461 | taattggatg | tccattggct | aactggagaa | gactcattgg | ttgtcaattg | gccaattgga |
| 20521 | gaagactcac | tggatgtcca | ttggccaatt | caatttggca | aatacagcac | ctactatatg |
| 20581 | caaaacacta | aactcggctc | tgagcagtag | aaaaacaata | ctagtctctg | taactactgt |
| 20641 | atgatcttgg | gagaatagtg | gaataatatt | aatgattaca | acacaataca | gaatagcata |
| 20701 | aggaacatga | atgaagtata | aaaacggttc | tgagaagtgt | cagaggacgg | agaaagaaag |
| 20761 | accgcattta | cttggtggct | ggttatcaaa | aatggcttca | tgaaggaagc | atcatttcag |
| 20821 | gtagaactag | gatgataagt | aacattttga | catggagtct | caggtgtgta | aagggcattt |
| 20881 | acccccacca | cacctgtgaa | ggaaacatcc | tatgcacagg | catggggctg | ggaaaaggag |
| 20941 | gagtgagctt | caagaactag | aaagtgacca | tttttggcta | gaatagataa | gggagccaca |
| 21001 | ggggataagg | ccagagagaa | ggccttatct | gcccttatct | ccttatttgg | cctcctgtgg |
| 21061 | ctcccctact | tattctagtt | aaaaatggtt | acttccttag | ggaaagccaa | gaagcacaga |
| 21121 | ccgaggcagg | gaggaaacgg | ggaactctaa | atgcttctga | gcagagggca | agaaggagga |
| 21181 | aaacctcttc | tggaggctga | tgcaatcgct | ccagggatag | gttaaacagt | gtttgcattt |
| 21241 | ggcaaatagc | tgcagaaatg | taacagaaga | gataggagag | acatcgtaga | tagataattg |
| 21301 | agaggccttt | aaactgacta | gatttggggg | acaaatagga | atcaggagac | attccagagt |
| 21361 | ttcaaatttg | ggtaagtagg | aaatgaagga | atcagtaaaa | cagagacaag | gcagaagaga |
| 21421 | agtcatttgc | ataggaagct | gcaaagtttg | tttttgtttt | taattttagg | aaatggtcag |
| 21481 | ttctccaggt | ggtcaacatc | ctccagaaag | aatttgtaag | tttgaggcca | acgctcacgt |
| 21541 | cagattcagg | aagctaggta | ttatctgagc | ctagtggcta | agatcaaaaa | gcttattcag |
| 21601 | aggaaaaatt | cagggcaaag | gcaaaacctt | ggccaatgcc | tacatttcag | tgttggagga |
| 21661 | gaaatagagg | caaaaaagag | aagaggtcag | agaagtgggg | agaaaaccaa | gatggcacag |
| 21721 | caccatgaga | tgcaggaRag | aagaaggccc | caacactggg | gagttgccaa | ttatgtataa |
| 21781 | gattgcacaa | aagttggaga | aatcagggcc | aagaaatggc | caatgttttg | aagcattaga |
| 21841 | agagccactg | gggtatttga | tttgagcaaa | cattttcagt | ccactggagg | gaatgttacg |
| 21901 | ctgaagttat | gctgtcatgc | tgaagacagc | taagtaagaa | agtggagttc | tgaactgcag |
| 21961 | tcaccacttt | caataccttg | acagtaataa | gaaagagaga | gagagtgtga | gacagagtgt |
| 22021 | gagagacaga | gatatatgaa | atgtttgctt | gcaaggtaac | aaggtccaag | aaggattttg |
| 22081 | ctactcacaa | agaggtagat | cctacagaat | tcatttgacc | catagaggcc | cataacagtc |
| 22141 | ccacaaaaaa | gaaagccaat | cccttctatt | gggtctccca | gccacatctg | tagccatttg |
| 22201 | aaatgaagaa | gcgttgagag | tgcttttgaa | gagttgcaga | tatagtgtgt | atcacataag |
| 22261 | gccattcatg | agggtgaggt | ggggataagg | agctagaata | gtaacctctt | ctctgccatg |
| 22321 | atcccaagct | ctttcctgca | aacatgctct | ttggtgttgc | cactaagaac | caactcccct |
| 22381 | ccctactact | ggaaaatctg | tcataaccag | aaatgcagga | aggagatggc | aaggtcaaag |
| 22441 | gacaggggat | actagaaata | gaggggaaat | gatgtgaatg | gaccatcagt | ttaaaaacat |
| 22501 | ttatttccca | gctgcctttc | gctatggatt | tatgctacaa | aatccatatc | attttctctt |
| 22561 | tcttggaaag | gaacataatg | acactatttt | gttgaatgga | ttgtcaagga | ctggcaaccc |
| 22621 | tgtgtaataa | ccacaaagct | attggcttga | atactgctat | ttatgtatgg | gttggcttat |
| 22681 | ggtctgaatg | cactaaacat | atttacaatt | cctcaagcac | acatgaaaga | ataatttttt |
| 22741 | taagtcattg | atgctattat | catcaagttc | gtgacactga | aatagcatct | tctatgaggt |
| 22801 | tttccagaaa | ctccatccat | ctagatcttc | agaaggctgc | cattataaca | aacttaactg |
| 22861 | gtcaacttac | tagaacactc | agaaattctt | attggctgaa | tgcacactga | gcatagatac |
| 22921 | tatctctgtt | gtgtgtatgt | gcagtgcctt | ggtgcttttg | aaatatttat | aagtgtctcc |
| 22981 | tcctaaggat | gcagacgtta | ctgtttacag | tctgaactta | tttaaggaac | caaacctgcg |
| 23041 | gatgcacttc | ctgttatagt | aatacctagg | tcttattatt | tataaatatg | tatgtgtctc |
| 23101 | ttgtattcat | aagacacttc | catctacatt | agctcatcaa | cactgtgttg | ctgagatagg |
| 23161 | tagaggaagg | aaagcacttt | tgtttcggaa | ttaaagctgt | gtctaggtgg | cattactcag |
| 23221 | gcagactctg | atgtaggcaa | cctggtcact | gtcacatctt | cctgggctgt | actggttaac |
| 23281 | actgtcaaat | ccacgatgtt | aatggtgcca | taatgagcca | ttcacatatc | gggacccagc |
| 23341 | ccatatgacc | catctcctgt | ctcacagaca | agaatgaatg | gcagaggtgc | aattataacg |
| 23401 | cacatcccta | attcaccaga | actctttttc | acagggagct | gctaacagga | tcccctgaca |
| 23461 | atccaaaatc | cccagcttgc | gtgaatgaac | taacagacac | atcagtttgc | tagaacagct |
| 23521 | gaagagggaa | atggtgatgt | ctggaaggac | tgaacctatg | tattctaata | gttactcagt |
| 23581 | agtgtcccca | gacacgagca | aagaaattgg | catggggaga | aacgggaaga | agaatgcaaa |
| 23641 | gctgtttcct | aaggaaaaaa | gaaatgacaa | ttccaatcag | tagtaaacac | acaaacaaac |
| 23701 | aaaatccctc | actgtttcct | tagcttagtg | ccaatcgtca | ttgaaaaata | aaattatggg |
| 23761 | ctgccctttg | tattaggttg | gcgcaaaagt | tatcgtggtt | ttggtggcca | aaaccacgat |
| 23821 | aacttttgcg | ccaacctaat | agcattgagg | aaggtgctag | cacagctttg | gtaatataag |
| 23881 | gaactctaat | agagccaggg | ctgaaatatg | ttccaaacat | ctgcgtcagg | cttggcagcc |
| 23941 | tgttcccagt | gtaactgatc | atggaagagg | gtgagaggtg | aagccaactg | gacttccggg |
| 24001 | gtcgagtggg | gacttagaga | acttttcttt | tctttctttt | tttttttttt | ttttttgaga |
| 24061 | cggagtctcg | ctctgtcgcc | caggctggag | tgcagtggca | cgatatcagc | tcactgcaag |
| 24121 | ctctgcttcc | cgggttcacg | ccattctcct | gcctcagcct | ccccagtagc | tgggactaca |
| 24181 | ggcgcccgcc | accatgccag | gctaattttt | ttgtattttt | agtagagacg | ggggtttcac |
| 24241 | tgtgttagtc | aggatggtct | ttatctcctg | acctcgtgat | ccgcccgcct | cagcctccca |
| 24301 | aagtgctggg | attacaggtg | tgagtcaccg | cgcccagagg | acttagacac | cttttctaca |
| 24361 | agaggtttgt | aaaatgcacc | aatcagtgct | ctgtaaaaat | gcaccaatca | gcgctctgta |
| 24421 | gctagctaga | ggtttgtaaa | atggaccaat | gaacaggaca | tgggtgggga | caaacaaggg |
| 24481 | aataaaaact | ggccccctaa | tctctgctca | ggtccccttc | caggctgtgg | aagctttgtt |
| 24541 | ctttccctcc | tcacaataaa | tcttgctggt | gctcactctt | tgcggYggtg | ccatcttttt |
| 24601 | tttttctttc | tttctttctt | tctttttttt | ttttttttga | gacagtgtct | ccttctgttg |
| 24661 | cccaggctgg | agtgcattgg | cacgatctcg | gcttattgca | agctctgcct | cgcgggttca |
| 24721 | tgccactttc | ctggctcagc | ctcccaagta | gctgggacta | caggtgccag | ccaccatgcc |
| 24781 | tggctaattt | tttgtatttt | tagtggagac | ggggtttcac | catgttagcc | aggatggtct |
| 24841 | cgatttcctg | acctcgtgat | tcacctgcct | ccgcctccca | aagtgctggg | attgcaggcc |
| 24901 | tgagccaccg | cgcccggcct | gggtcggtgc | catctttaag | agctgtaact | ctcactgcga |
| 24961 | aagtccccag | ttccgttctt | ggtcagctag | accgcgaacc | caccggaagg | aataaattcc |
| 25021 | ggacacaagg | gggcgcgcgg | ggcatgcgta | aaagcctgct | ccggggccgg | gcgtggtggc |
| 25081 | tcacgcctgt | aatcccagca | ctttgggagg | ccgagatggg | cggatcacct | gaggtcagga |
| 25141 | gtttgagacc | agcctgacca | acatggggaa | accccatctc | tactaaaata | caaaaaatta |
| 25201 | gctgggcgtg | gtggcgtgtg | cctgtagtcc | cagctacttg | ggaggctgag | gcaggaaaat |
| 25261 | cgctggaacc | caggaggctg | aggttgcagt | gagccgactt | tgcgccactg | cactccagcc |
| 25321 | tggcgatgga | gcaatactcc | gtctttaaaa | aaaaaaaaaa | aagcctgcac | cgggacccag |
| 25381 | tctctctcca | gacaatccgt | agcccttaac | tgtgctgctg | ggaaccagag | gccccaaaat |
| 25441 | tcaaaatcag | gtgcctctga | ttaagctaga | gggagtgagg | aggagcggat | gtggccagat |
| 25501 | tctctgaatg | tggacatgca | ggcacccact | ccaacaccca | gggagtggct | gtcttaagtg |
| 25561 | ctttctgaaa | ggttgtcatc | cagacaaacc | acccacttca | gcagttattg | aaattgcccc |
| 25621 | tttcactctc | tctttgctga | atttttgctg | atctcttctc | catatgacct | actgtgggtc |
| 25681 | acagacgctt | ctcagggaca | cattgcggtt | tttcacctca | actcttcata | tacctcattt |
| 25741 | tgagttcaat | acaagtgtca | gctgtttaaa | ctagtgttta | catgtgcaaa | gcattctgtg |
| 25801 | ggacagcgaa | aggtagaaat | gtaagtaaga | catggttttt | gtcttgctgg | ggtctgccac |
| 25861 | ccaaacgaga | gatgaacacc | tgtacagtta | gaatgaggac | tctacggaga | tgggtgctgg |
| 25921 | aaggcaaaat | agagtgctgc | agtcagactg | tcacattaac | tggaatttgt | caacttgact |
| 25981 | ggctgccatc | tttccaaaag | agttgatcta | gaaatttaca | ttttatttat | ttatttttga |
| 26041 | gacagagtct | cgctgtgatg | tcccggctag | agtgcaatgg | cctgatcttg | gctcactgca |
| 26101 | atttctgctt | cccgggttca | agcgattctc | ctgcctcagc | ctcctgagta | gctgggatta |
| 26161 | caggtgcgtg | ccaccacatc | tggctaattt | ttgcattttt | agtagagacc | gggtttcacc |
| 26221 | atattggcca | ggctggtctc | gaactcctga | cctcactgat | ccgcttgcct | tggcctcgca |
| 26281 | aaatgctggg | attataggtg | agccaccgcg | cctggctaga | aatttacatt | ttattttatt |
| 26341 | tttaccatct | tttctgtgac | caaatgtgtt | aacaaacaaa | tgcccacttt | ttcactttat |
| 26401 | tggagaataa | aaaggacaga | caactatgaa | ctgagatttt | ttttaaaaga | taaaacagtg |
| 26461 | atcttagtta | gccaggggtt | accacatcta | aaacagccta | atctcttatc | agaccaagtc |
| 26521 | agccttagct | cctccaggtt | tgtagagact | tgtgtctgtt | ttttaatctt | taagtcaaga |
| 26581 | gtgtgttcct | tttttcttta | aatgtaagaa | tgtatgcctt | taaatatttt | gaagtgatct |
| 26641 | atgtaagatt | atgattttga | ccccgtctta | attctttcca | tgccacttcc | tgggttgctt |
| 26701 | gtatcttgca | taatagctgc | cctgctggta | gcgtccaaca | gttaagagag | tcagacaagg |
| 26761 | acttggaatc | gtgactatat | tgactggcct | ttgctatctt | cggctattct | ggagaaatat |
| 26821 | gagaacttga | attgagagac | agttagcatc | ctctttcagc | ccgggactgg | aagctctctc |
| 26881 | cgtgacatag | cagtctatca | tagaagcagc | aggccatcac | ctccaacctg | gctggctgct |
| 26941 | tgtacaaagt | atccacccag | ataaaacgaa | atacactgga | gcatctgttt | cttctcagaa |
| 27001 | aactcttcca | tttctcctag | gaacccagtg | cctgagcagt | ctccctttaa | cccttacaga |
| 27061 | gccaccgtgg | caaatgcaag | gatcgaaact | gtatgaatga | gggcgctgtg | ggagaagaac |
| 27121 | acttccctgc | tatttacctg | agattaagta | gatgaccagc | gcttggaagg | gaatgtggcc |
| 27181 | ttctcacctc | tgggcctgag | aacaagcact | ggacacagtg | gtatctaata | tgccctttta |
| 27241 | ccaattgaac | agatcaaacc | catgtgtggc | ctggttgtca | ggctttaaat | aaacattgta |
| 27301 | tttatatgcR | ctttgtgctt | ttaaactcca | tcaacaaaaa | gcactagaat | ttagatattt |
| 27361 | actccccagg | atttccattc | ctaagttaag | gcaagttttt | atttatttat | ttgttttttt |
| 27421 | aaaaactgaa | ggatagagtc | tcgctctatt | gccgaagctg | gagtgcagtg | gcaagattat |
| 27481 | agctcactgt | cacctcaaac | tccaaactcc | tggcctaagc | gatcttcctg | ccttggcccc |
| 27541 | caaaagcact | gggattacag | gtgtgcacca | ccacaattca | ctaatttttt | attttttgta |
| 27601 | gagagagggt | cttgctatgt | tgctcaggct | agccttgaac | tcctggcctc | aagcaatcct |
| 27661 | ctcacctccg | tctcccaaag | tgctgggatt | acagacctga | gccaccactc | tggccaagtt |
| 27721 | tcaattgagt | ggaaaaccac | caattggttg | aaagtttcta | tgcacagttt | tcacaagtgc |
| 27781 | ttatttaatc | tgaacttaaa | gttcaaaaat | taagtccttc | ctcagaatta | gttggtttat |
| 27841 | acttaaactt | tcctgagaac | ttgttaataa | aagagttgct | tgaagaaaaa | ataagctggt |
| 27901 | aacgtttcat | gagaatttcc | tcattcaaaa | agcacatcca | tgtgtgactt | tgatcacaaa |
| 27961 | gacagaaaga | tttgtgacgc | agcaactatt | tatttgggtt | ctgttcatat | gacttttccc |
| 28021 | agcttcaggg | atctcagctg | tcaaatgagg | atgagagaaa | agtgagtgct | catccccata |
| 28081 | atgggcacat | attgaagact | caacagcaag | agcccggcat | aggttggaca | tggtggctca |
| 28141 | ggcctgtaat | ctcattactt | tgggaagctg | agacagagaa | tggcttacct | caggagttcg |
| 28201 | agaccagcct | gggcaacata | gtgagaagcc | cccccatcct | tctgtgtttt | aaataaataa |
| 28261 | ataaatatgt | ttttcaaaaa | aaaaccagtc | ataattattc | cttcccaaac | actctatgat |
| 28321 | tatttaagtt | aaagaagaaa | aaaacaacat | gttgaccaaa | gaagatctag | caataaagac |
| 28381 | ctgggcttga | tgaggaggaa | gaccacgtta | taaggcaaaa | gttctccatg | aacttgaact |
| 28441 | ctataaagga | attgagtaga | cggaagccaa | aatattctgt | ttgttttttt | ttcttcctct |
| 28501 | gctcctctgc | agaacttagt | agagctctga | atgaatgtgc | ttcacttcct | aagctatcta |
| 28561 | tgaattgttt | caaccataag | acactactaa | ctgccctaga | taccagatac | aggccaatca |
| 28621 | accagagaag | agcaggagaa | ccctcctctc | aaggtgcaac | gaactgccaa | gccactgact |
| 28681 | cttacaatca | ggatggatga | caaacaccca | ggcaagaggc | atttggaacc | tgatccaaat |
| 28741 | gaatttttgt | ttcttgttgt | tctttaggct | taatgatcag | tctagatgaa | tactaccact |
| 28801 | gcaattagtt | acgtttattt | tttagcttcc | tttacacaca | catatgtgtt | tgtgtgtgcg |
| 28861 | cttctgtgga | gccatgtcag | caggcatgca | agtatcaagt | tttatgctgg | aaagaaagga |
| 28921 | gctgagaatg | tgcatgcccg | ctgaaggcat | atgtctcact | tggatcagtt | aagtgcctgc |
| 28981 | tccctaaagt | gaaatctcgt | tggaaatgcc | atgcacatgt | tgattcagtc | tgataagatt |
| 29041 | tcaaccaacc | ttctccttta | gctcgcaaaa | gttgcttcct | ttaaattaca | gaaatgccta |
| 29101 | acacaaaaca | acctcgtaat | actcaaaatt | cattatgatc | aaatgtgatt | gcttatacta |
| 29161 | agggataata | aagaaaatta | attttattaa | acacacgagg | ctctgttcaa | attaaatttg |
| 29221 | gtgctaatgg | ggaaaaaaat | caaaaggaag | aaagcagata | attatctgcc | tgagtaataa |
| 29281 | ccaaggatgc | tgcaagtggt | agtaacaact | ttggccagca | gttcttctga | ggagggttca |
| 29341 | ttcttccacc | aatagttttt | taattgaatt | gagcttggtg | gtggtgcctc | tcacattgag |
| 29401 | aagtcaccag | agatcaaaac | cagaaataaa | aagatgaaga | aaaatacggc | aaaggagaat |
| 29461 | gtttctgaat | ttgcttgcac | ataaaatctg | cataataaag | caggctccag | attgaaaagt |
| 29521 | ttgaaacaaa | gacaagatat | ttccatcagt | agccactgaa | aaaaattaga | aaatgcatca |
| 29581 | gtaggattct | gctttttgat | tagaaatagt | acctattttt | tctatatcca | ctccaaagac |
| 29641 | tttcactgtc | tatgttcaag | gatagtaccc | acaggcttct | tttttttgag | acggagtttt |
| 29701 | gctcctgttg | cccaggctgg | agtgcaatgg | tgcggatctt | ggctcactgc | aacctccgct |
| 29761 | tcccaggttc | aagccattct | cttgcctcag | cctcccgagt | agctgggact | acaggcgtgc |
| 29821 | accaccttgc | ctggctaatt | tttgtatttt | tagtagagac | ggggtttcac | catgttggcc |
| 29881 | aggctggttt | cagtcgaact | cctgacttca | agtgatgcct | gcctctggct | cccaatgttt |
| 29941 | gcgagtgagc | caccgtgcct | ggcctgtaca | tacagacttc | taagactcta | gttgagtttt |
| 30001 | acctgatttt | gaaatgtata | aaaatgaagt | cacagaagtt | aggacagcag | ttgcccttga |
| 30061 | gggcaaaggt | tggtgactaa | aactgacatc | agagggcttc | taggaagctg | gaaatgttgg |
| 30121 | cctctagatt | ggttttgtgg | atgtatccac | cttgcaaaat | tttatcaaat | cttatacttg |
| 30181 | ggttgtgtat | ttttctaaat | atatattata | ctttactaaa | ggtatatatt | cttctctatt |
| 30241 | tcatatggaa | aatagagatg | tagtttggct | taaaaaactc | agttttccat | aaaacaagga |
| 30301 | ttttgtttta | gtctaagtga | tgctctaaaa | tacaagacta | gagtgaggta | cttcccttgg |
| 30361 | atacaaaaag | ccaaaaactc | agtgattaag | ataaatagta | ttttaatgta | acatttttta |
| 30421 | aaaatccaaa | ttaatcctaa | aaaatccatg | ctagtaataa | taaagaaaga | ttaaaacaga |
| 30481 | aaatattact | catattactc | aggaaaacag | aaaatggaac | ttacactcca | aaaaatgcca |
| 30541 | ataagtgtaa | atgtgcagga | ataaatgtaa | atgtgcggga | ataatcagat | ttgagaaacc |
| 30601 | aagggcagat | tatttttcat | tccataagtt | tatattctct | gaaaataaaa | gacattagca |
| 30661 | ctgtttcaaa | gtttttaata | atttaggtaa | tttaaaaact | agcattaaag | gaacaatatc |
| 30721 | tgaattatcc | caacaaaatt | ctgaaacgaa | tatcatctct | atttgcttat | aatttcacta |
| 30781 | tgattaaact | tcttacgaag | ttacaagtat | gcgatcttcc | tttgaacgca | ctgagttagg |
| 30841 | aaaaacatgc | ctattgttgg | ttacacagag | gagagattac | aaatctaaat | catagttgaa |
| 30901 | aaaagcaact | gtgccatgat | atgaaacagc | actgtcaaat | cagacactga | agtcggtaag |
| 30961 | taagattttc | ttcttgtcag | taggaaaagc | actactgggt | ttgagtcccc | caatgctacc |
| 31021 | acctttttag | acttttgtta | gagctttgca | ttcatttcct | taatgttttt | aacccaatgt |
| 31081 | ctgtgaaatt | tttattgctc | cccggatggt | gacatctgat | ctgaatgtaa | ccatacacct |
| 31141 | ctttgctcag | cagtatttgt | cacaaaccat | tataaattat | catgctgatt | gtgtgcattg |
| 31201 | ctgggaaact | ggagaagttt | cataaattct | aactaccata | tcaatgacat | tcttgtttat |
| 31261 | tatacagata | taaactttta | tgagattcaa | gatgtctgcc | ttaccactga | agaaaacttt |
| 31321 | agtggagtaa | gatctaacta | gttcgatgta | catactgaaa | accattttca | atgtgcaatg |
| 31381 | aacatgtgtt | tccgaaaact | ctccactggt | cacttcttta | tttcttttct | atttttaatt |
| 31441 | atgctttgac | acttcacaag | tgtgcaggat | tttgctggca | aaaaagaaat | caacattcct |
| 31501 | tcctctccct | caacaccttc | ttcggtcaga | gcattctcta | gaatcctctc | aatctaataa |
| 31561 | taaaatcatg | catatatata | gctgggaaaa | aatcctagtt | ctgtgttagc | cctgcagaca |
| 31621 | gacgctccac | tggtctgttg | atcgactgat | ttacaccaca | tcttgttcaa | taaaagaatt |
| 31681 | aaggggttat | agtattatgg | ttctgatgct | tatttgtaat | gaaacctcat | tcagtgctgg |
| 31741 | catagacaca | gaatgttgca | tgttaaaact | atggaggcac | aacttacaca | tttaattttc |
| 31801 | ctaacgaagt | atccaccttt | gacagccact | gcaaaaactt | caccattttg | caaagtatga |
| 31861 | tccagaatta | actcatgaac | tgggtcagtc | ttggtgctat | tcacatgaga | agttgtgact |
| 31921 | gtatactgta | ttgttaagat | tatgaaatga | tgttttttac | ctaattatac | ataaaacaat |
| 31981 | aaaaatctaa | ataaaaaatt | tttttttttc | taccaggact | atgtagctgg | tctaatgact |
| 32041 | taaccatccc | tatactctac | aaaagaaaac | tgtttacttg | ggtttcaaag | atgtttcaca |
| 32101 | ataagcaaat | gtgctaaggc | atcgatttct | taagctttct | cagggtggaa | gttgactata |
| 32161 | caaatactga | ccatttcaag | agaaaatgca | gggtgtatca | tgtgcaaatc | atctcattac |
| 32221 | tgggtcaata | gatttagttt | tgacgtatgt | ttagtttcat | atttccaaat | gttctaaaaa |
| 32281 | tcaccaactt | gttaaagata | tcataataat | gataacttat | tttgagggtc | acttttacct |
| 32341 | tttaaaaagg | ctttcctatc | tactacttga | tcctactagt | aaccctatga | catcagcata |
| 32401 | gcaaatgttt | tttactgcca | acagacatat | aatgaagctg | gaacaaggaa | aaatattaaa |
| 32461 | tgtcttaaat | aagatgaata | gtttgttctt | aacaagagtg | ataactaaaa | gctcaccagc |
| 32521 | tttcagaatg | tggttttaac | tgtgcctagt | tttgtgccac | ctccaatcca | tttttgtaaa |
| 32581 | tagcctgata | taaagtacaa | aaaaacccta | ataacaaaac | tatgtttgta | ggaatctaat |
| 32641 | ctaaactttg | aagtctatga | atttgttctt | caaataattt | ctggtgttcc | caggtttttt |
| 32701 | ttttccccct | agggcattcc | aatcccctgt | aaaatttaaa | tctgtggatt | cttctcaaag |
| 32761 | ccttcacaaa | aggaggtaag | tattttgcca | catttgtaga | atctcaaatg | cacccttcaa |
| 32821 | cactgagcac | tagtcattca | tatgaatcag | cttagaaaga | tagtgaaagg | aatgccttat |
| 32881 | gctggtcttc | ctatatcttt | ctggagtttc | tcattgataa | aaagctcaca | atgcaacact |
| 32941 | tcaaaacaca | gcaggaagat | attttccatg | gatctaatct | ccattgactt | catcaggcta |
| 33001 | aatataacag | cctttcagct | ttggccagga | gatgRcacct | tcacagagag | caaagcctca |
| 33061 | gttgggacaa | ttaagaaatc | accttactct | gtagcattaa | aaaaacaaac | aaagtcacca |
| 33121 | ttattaagat | tgttatgtaa | catcttcttg | gccatgtcag | tttctcaaat | actttgctga |
| 33181 | tatcactaaa | actgtcattt | tctgtgaatt | acaatcatct | tttaatgtac | tttttaaaaa |
| 33241 | gaaaactaac | caaaaaggtg | ctcctttaat | cccattatac | cccacctgac | accatgatat |
| 33301 | gacaaatcat | tatttaaggt | ttactgtgag | ctagacacca | ttgtaatact | gtgagatgct |
| 33361 | gttgtaataa | ttagtatacg | ctgattattt | ggaaactgat | catcagccta | tggatttatg |
| 33421 | ctaattctct | aggcaccaaa | atgttgaatg | aaccagatga | ccttatttcc | ctgaccatga |
| 33481 | aaagctagtt | tactacacca | actcaacagg | ttgttgagcg | ctttctacag | gtaggaagat |
| 33541 | ttttatatta | ctctgttctt | tcccagaaga | aatgtatgtg | caaatagaat | tgtaagccct |
| 33601 | ttacatatat | taagtgattc | aactggcttc | ccttcttaaa | tttctactcc | agttgtgtgc |
| 33661 | tggatctggc | gcccaccagc | tcataagaac | caactgtgca | catttctttg | ctagtctggg |
| 33721 | cacagtgaca | tcacatggtt | agtttgaaat | tggctatgat | gagagtattt | acaccacaga |
| 33781 | aattggcatg | tgctacatac | aaatgaggac | ttgactcgtc | ttctccaaag | agccagctgt |
| 33841 | taaacattta | ccaccacatc | actgccaata | actagtgatt | attctgaatt | ctttaagtat |
| 33901 | tatattacac | gtattgatgg | tcatgatttc | cagattagaa | attagtaaac | gtcagtttat |
| 33961 | tgaattctaa | cagcacttta | ttttttggaa | taatgttatt | acttgagtat | gttttaccag |
| 34021 | agtttctcct | atagcagttt | aaacatggtc | aaaaagcaaa | tactaataac | tactgctact |
| 34081 | actaatgaag | taaatcaaat | ataaaagaaa | acaagtctcc | agattgtcta | ggtgattgac |
| 34141 | actgtaaaaa | ccaccataaa | gaatattagc | aaaaataaat | aaatcaatac | aatgaactct |
| 34201 | ttgtttccaa | gcagtcactg | gaacactatt | aatggtaact | tccatacaca | attaaaaagt |
| 34261 | caatataatg | ccctgaggca | cttctgaatc | acaaagcaaa | gataaattgg | aattggtgta |
| 34321 | attttgttgc | actgtagtat | acactgatta | tttggaaact | ggtcatcagc | ctgtggattt |
| 34381 | atgctaattc | tctagatact | aaaatgttga | atgaaccaaa | tgacctcatc | tctctgacca |
| 34441 | tgaaaagcta | gtttactaaa | ccaactcaac | aggttgtcga | gcacttccta | caggtaggaa |
| 34501 | gattttgata | ttactctgtt | ctttcccaaa | agaaatgtgt | gttcaaatag | actgtcttta |
| 34561 | acaatgaata | tacttgcaaa | ataatattta | ggtccagcgg | tagcagtagt | ctgtagatat |
| 34621 | catatacggt | tacaccaaac | tgacccactt | taaaacttgt | gaaaacctct | cagtccctga |
| 34681 | gaaacacttt | agaattgaga | tttaaatcga | ttatttttct | ttgataaaac | tatttatata |
| 34741 | tgtacaaact | taaaagatgg | atattcaggt | tcaagtctct | ggagactaca | ccactttcag |
| 34801 | tgggtcagaa | tgggataatg | tggacaagac | agagacaggg | ggtctgttgg | gtgttaagag |
| 34861 | agacgagcaa | acatgtatct | gagtggactg | ctacgtgcaa | ggtaaaaaca | tactttcgat |
| 34921 | actgtggaaa | gcaatatcca | cataccagtt | ctttcacata | aagtgaggaa | atataatttc |
| 34981 | gactgtctct | atagtgagaa | tgtcttcata | ataacgaaat | tgggcataag | ttgatgttta |
| 35041 | gaaacttaac | aatagaaaga | gatagcaaga | ctaagcaggt | ggatgaaaca | ctgatgtgta |
| 35101 | tttgagttcc | cattgtattc | atcgtgtctt | aacaattatt | ttctagcatt | ttccataaaa |
| 35161 | gtattttaat | cagtaatcca | ccatttacaa | aacccctcat | ctggcttatc | ctgtaggcct |
| 35221 | tattttattc | aatttgcttt | ttccatgtca | aacttactta | gtaagatgaa | tgcagttctt |
| 35281 | aaatcataag | atgtgccaca | gttcaatata | tctcaaatag | tgagatctcc | aatcacacta |
| 35341 | tctgtggatg | cgatgtatat | tagtgatcca | ctgtatttcg | taaaaataca | agtgagattt |
| 35401 | ttgagtttct | agttcttccc | ctctccagga | aaaaaaaaaa | aagaaagaaa | aaagaaacat |
| 35461 | acatactgta | tacatttctt | aacaagagtt | cccaaaccaa | aagtgctaac | aggaaggagt |
| 35521 | ataagagctt | acacatttaa | ctataatctt | cctggtttgg | ggaactcaga | aatcacttaa |
| 35581 | cgtacttcaa | tttccatacc | attgacatat | aatctggctg | agattggaat | ccctcctgga |
| 35641 | tccatttata | atctacattg | tacacacYga | cttccccccc | gtagacaggc | gtgaccacta |
| 35701 | gatggtgctg | tggtgccctg | ttcaaaaaca | ttcccactta | cttgcagtct | ctgtcctcca |
| 35761 | aatcgaagtg | agggttgaag | ttgatcataa | ttctctggta | tgggtccgga | gcctgaatca |
| 35821 | gccattcgca | tttttcactt | gggtgataag | aatgaggata | accaggagat | gtaaggtacc |
| 35881 | cggggctttc | aatttttata | gtatcgccac | atttatctgc | aatgaaagta | agattttagc |
| 35941 | agatctttcc | tgacaacctg | ttaggtaatc | tagctttata | tgtaaattac | tggttattaa |
| 36001 | acattcctta | actttttaag | gggaactcta | aaaggtcccc | gtaagtcctt | cgtgtggctc |
| 36061 | tgagtgcaac | actccaggaa | aaaacactct | ttgatctcag | gaaggttgac | tcaggcagaa |
| 36121 | agccaactga | gatctgagag | gactcttccc | cccagccccg | aagtttcttc | ctaaggaaaa |
| 36181 | ggtcttctag | cattaaaaac | attaaatccc | ctcaattgat | gttaacatgg | tcccctggga |
| 36241 | gcttctgcta | gacttaatta | tcccccaaga | agtgtaatca | cattaagtag | caagttctaa |
| 36301 | gtggaaagtg | actgaaagat | gctgttcgac | cccttgtgtt | gttttttgat | gataagcctg |
| 36361 | gtctatgtca | ttgatcagtg | gggattgtct | ttgattggct | taaagccaaa | ccttttgttg |
| 36421 | ttgttgtttt | atctgcccat | aaatctaagt | gattttgagt | tatctctgct | cttgagatcc |
| 36481 | aaatcaacag | agaacaaagt | atatttggtg | aagcagaaat | aattctgctt | tacttctcca |
| 36541 | agtaaagttg | ttcaagcttg | gagatcggat | caaagcaacg | gggcagaagg | aaaagtaaat |
| 36601 | agccacatgg | cattaattct | aattacctaa | atgttgggtg | aaaaataacc | caccacacag |
| 36661 | gtctgggtat | cacatatggt | aacccacatt | ctcacattct | gggaaaggtg | agctaagtag |
| 36721 | ttttgagcct | tcatttcaaa | aatgctcaag | tgaagccaaa | taaaccaagt | aaaccagtct |
| 36781 | ctgggctgcc | tatattgttc | agcatagatt | ctcattgtca | cattttatga | tggatttcat |
| 36841 | actggagcac | ctcagcctga | aatgctgcca | catttattga | tgcttaattt | tgagcaataa |
| 36901 | aaattcggtg | cataaactat | ataagagaaa | cggtatgaaa | acagtgaaat | atataagcct |
| 36961 | atcaatcaca | aattttcagt | aatggtagat | gtagatttta | acactgcatc | taatagattc |
| 37021 | cctgtggaaa | gagaatggca | ttctttgctc | tcagcatgaa | tttataaatc | tacagaattt |
| 37081 | tattacagga | aaaccaggtc | aaatggaaat | gaaagatgaa | gtgcacatca | aaaatgcata |
| 37141 | aagctacatg | cattacagaa | atcatttccc | ataaattaaa | aaaagaagac | aatataagtt |
| 37201 | atattgtttt | tctcaatcgc | taggtgcttc | tgactttgtt | ccttagactg | ggggtagtct |
| 37261 | tatgctggtt | tcctttgcac | tggcttgtaa | aagtgtaagc | tttgccagga | agaaatgatg |
| 37321 | ttgcaactgg | agttcaaaat | ctcagctaat | gatcatgttg | gactctgtta | tgtgattctg |
| 37381 | ctgagactct | tgcattattt | tgaatgtgca | gaaaacttgg | aaagtgacct | cgtctcatta |
| 37441 | agtttcctct | agctctggac | aaacatcatg | gtagtgtctg | gcatctgtta | caggagtgaa |
| 37501 | ataaaacagc | actggttcta | gtcactcgac | agagtcactt | cagaagacat | ttaggtggac |
| 37561 | ttggtaccac | accagagctc | tttaatctgt | gacttctgag | aaagtgacag | tgtaataaga |
| 37621 | taatgtggac | tgctagactc | cctggctctg | ttcaatctct | ctagcagttg | attaagaaag |
| 37681 | agcagtttct | aatgactctg | gaccattagt | cctaaataag | gttaatgaat | taatttagca |
| 37741 | gacactggtt | actcagagga | ggtggacatg | gctttgagag | gtctgctgag | ttacagaggc |
| 37801 | attttctggc | tctggtcact | tcaagcatag | cgacatggct | tttgctggta | aagctcaaca |
| 37861 | actaaagcca | gattttcagc | attccccaga | tttctgctaa | gaaaactaag | aagtgtgtgt |
| 37921 | gtgtgtgtgt | gtactgcata | tatatttgtg | tagttcacaa | cctaaagtct | gatagctctt |
| 37981 | tgcattgagc | ttcaaatcta | tcttgtgaac | acccccacta | tggatttagt | ctcaagagtt |
| 38041 | gtggaggaca | gggttactat | aatttctagt | ccaaccattt | gagctctttc | agttggacga |
| 38101 | gaatgctatt | tcattccctg | ccagagtata | gaaagatgct | accagctcag | gttcattaat |
| 38161 | aaattgaatt | ctacattcat | aattttttgt | gtcttgcttt | cctgccagat | tatagaaaaa |
| 38221 | tgccatcaac | ttgggctcat | taataagctg | aattccatgt | tcataatttt | ttttttNgca |
| 38281 | tgtgttttgc | tttaagcatc | tcttaaaata | ctgcttggta | gctgctgagg | gaaatccatt |
| 38341 | gctaaaatca | caaaaccttg | aaaaagcata | ttagagagat | taacttccat | cataaagatg |
| 38401 | tgatttaaat | tcctgttggt | atgaggccct | ccacttgcgg | tctcttatta | aaacaatata |
| 38461 | ccacagtaag | tagtggatgt | tcttttcttc | taattactgt | gattgaaatc | taagccagac |
| 38521 | aaaagtaaat | atttgatcat | tagttaataa | caattgctga | cgacttactg | taaagataat |
| 38581 | tgcataccct | tgcctgcact | tactttacag | tgtgaagtca | aacatgccta | caaccagatc |
| 38641 | ccattttaaa | acacattatt | taggttacat | ggtactgttt | agaccccagt | atagtttttc |
| 38701 | atttacttaa | cagtgagtga | ttttaaaagc | taagtggaag | ctgcaactag | cttagggatt |
| 38761 | ttcagatatc | ctggtgcctt | acactgtata | tgtcagattc | ctgtattgat | actatgtaac |
| 38821 | tttcagtcat | actccagaca | gattacggaa | ggtgtacaaa | ggcgggggag | ggagtttcat |
| 38881 | caacccacaa | accgatatct | ctggccgtca | cccaccagag | gaaacggctt | aggggagcag |
| 38941 | caaaggaacc | aggaagtaag | atctcctggt | aaagtgttca | ttaactttca | acagccatca |
| 39001 | gatttttgaa | agggagtctc | ttctaattgc | ctttgggagg | aaacattatt | aagataattg |
| 39061 | gcatgctgat | ctgggaaggt | agacagctct | gtcttgtaac | agaggtaagg | aaaagcccca |
| 39121 | cagcccaagg | atcaacgtca | ggagggagcc | caaagacctg | aaatcctcct | gctctaaacc |
| 39181 | gcctgatctc | atttcttctt | ctctcctccc | agataaaagt | ttccttcgcc | cgggagtcgg |
| 39241 | ttgttcccgg | ctgatcccgg | gaagccccgc | ctgagccccg | gtccggggcg | ggcagctggg |
| 39301 | agccggggcg | ccgctgtcac | ccgcgcctct | gcctgtcact | taccgttgcg | aaaagcgccg |
| 39361 | gccggggcga | ggacgagggc | gagcacggcg | cagaggagcg | gcagccccct | ctccattctc |
| 39421 | ccttctccgg | gtccgcaggc | agacgcggga | gaacgaggac | gtggggggaa | atgcagcaaa |
| 39481 | gaggagaatc | taagcgatcc | gaagagcccc | aactccgcct | agagctgtac | aatcctcagc |
| 39541 | ccgtcttgga | gaaaagaaag | cagcgaggca | atgcctggat | ccgagaggaa | cgcttctctt |
| 39601 | tttgtgtctc | aagtcgcctg | catcctgtca | tttagctccg | gcttcctctc | ccttttccca |
| 39661 | cacttgttcc | tcttccagga | gccactgccc | gggccatgtc | tcaaaaaaaa | aaaaaaaaaa |
| 39721 | aaaaaaggcc | gggggggggc | tgtgggtggg | agggggagga | gggaggtagg | gcaacacaca |
| 39781 | ccaaagccaa | tttccaggaa | gaaaaaaaaa | aaatccggct | tgtttctgga | cccgttggag |
| 39841 | ccgcggagct | ggcgcccagg | ggaggtccgg | gtgtctgcgg | gaaggagggg | aacgagcaat |
| 39901 | gaaaagatga | gcgggagaca | gaggagtttc | accaactgca | cccccgcctc | ccctcctgcg |
| 39961 | gcctcccccc | actccggagc | ccttccctcg | cttcctcccg | cccctctccc | cgcttgggct |
| 40021 | cgcctgtaat | tgcctgacag | gagactggga | ggaacaactt | tccctccgag | tgctctcccc |
| 40081 | gtccgtctgt | ctgtctttcc | cagagaccct | gcagggatct | ccgcgggaag | gaaggcgctg |
| 40141 | ggagtctgcg | ccccggtcgc | gtgggtgcgg | gcgtcggagg | aaggggcgcg | gaggtcagtg |
| 40201 | ccagccgcag | ggtctggcca | gcccacctcg | cgtgctcgct | ctgcacccct | agagggagag |
| 40261 | ctgggcagct | cctgcaggcg | cagatcccgc | cgccgcgcag | tgcggagcgc | ccgggaggtg |
| 40321 | gcggctgctt | ctcgcaactt | caaatcccgg | ggcccgcgcc | ctcggcggcg | aggaacgcag |
| 40381 | ggtcaccccc | tgccactcct | catcgcgcca | tcctgagacc | tgtggcgggg | agcagcgggg |
| 40441 | cacccctgga | cgcccctgcc | cagagctgga | gggcgaggag | gggaaagccg | ggctggagtg |
| 40501 | ggagccgcac | cgcgaagccg | ggcgaggggc | agcagtgtcg | ctgtggtgtg | aagcgaagac |
| 40561 | agaccgcctg | ggcgggcgag | gccgggctgc | agccccacgc | ggcctccctg | gcccgcatca |
| 40621 | gggcgcgcgg | ctgggtgccc | tctgcgcgcc | gcggaggctt | cattgtttcc | cgagatttgg |
| 40681 | tgcccgttcc | tctggggcag | agggaacacc | ggagggtggg | aggtgctgcg | ccttccaagc |
| 40741 | aggggggcaa | tcgccgcccg | accccgccca | accttgtctg | ggagcccagg | tgctagggag |
| 40801 | gggtgtggaa | atcaggggtc | gggcggcccc | cgtttctctg | tgcatcccga | gggagggaaa |
| 40861 | gagtggcggg | ggaggggtgc | tcttcgctgc | gccctctcag | tgtccgctcc | cggccacaga |
| 40921 | caacgtgcac | gccggccctg | gagctcctgc | cacccccact | tcccacagcg | gctcgtgccc |
| 40981 | ccagcccgct | ggatgccccc | caggcccgtc | cccgagggtt | ttccgagaag | tgctgcagga |
| 41041 | agcgtttgta | gatgcgaaaa | gcaaggacat | ttctgggaag | aaacaggttg | cggtcaccga |
| 41101 | tcaggccccg | gagaagggtc | agtgcccacg | gggcacctcc | cagggagttt | gtgggtaagg |
| 41161 | tgaaaaggtc | acacgcctcc | tggggattgg | cgtctacaca | cacagccaaa | gtacagcctg |
| 41221 | attcctacct | ggcacaaata | gctggccaaa | gggaggtagg | aatgctggca | aggagactct |
| 41281 | ggtcaaaggt | tttggcggaa | gaaaaagggg | cgggcaggga | gaaagcccaa | ggcgcgcggg |
| 41341 | ccccaggcca | ggactgcgcg | ctcgcgactc | gggctgggag | cagtgacgtg | cgcccctcgg |
| 41401 | ggtgcaggcc | gttcccgggc | cagtgggagg | tgcacccacg | gagcccgcac | tcgccactct |
| 41461 | cttgccccgg | gtcaagcctg | tcttctcctc | cccacgcgcc | tcgggattcc | cagtcgccaa |
| 41521 | gttggaaact | cggccccccc | gagcgcctct | ttcctctgcc | agtttcctcc | ttcttctcag |
| 41581 | ctgtcggacc | cctgggaggc | gcggggtggg | aagtgggcac | cgaagggctg | ggagttaaat |
| 41641 | gcctaagttg | agcggaagga | gcactgtcga | agccatgggc | agccctgcgc | ccgggcaggc |
| 41701 | gggcaggcgg | gaggcgaggc | gggggcgccg | accgcgccca | gcgcagaccc | cgggggagtg |
| 41761 | agcgccgcag | aggcagcctg | ggctctcggc | tactacccgc | gcgcggtcgc | cgcctcccca |
| 41821 | ggtgcagccc | cgccagtccc | agcccccggc | tgcgccgggc | gagcggcatc | atcagattta |
| 41881 | gggtgacccg | gagcgacctc | ggtccaggct | cggagggaga | ggcgcctctc | aggacctggc |
| 41941 | aggttgacag | ggaaggggaa | ccgggaggga | gcggcggccg | gggcggcctg | gagtctcgga |
| 42001 | ggcccgcctg | cgtcctgggg | ggacccgacc | tccgcgctcg | ctccggagcg | cccgtttgga |
| 42061 | tagctctgag | cctcggcggg | gtgagcgagg | gggcacacga | gggagacttg | agctgtgttg |
| 42121 | tttttagaaa | actgccgcca | atctctcttc | ccctttccct | gctgagtgtg | cattttccag |
| 42181 | cctagacccg | ccaggccagc | aaataaagcc | accggatcgt | gcactccgga | gcgactgccc |
| 42241 | ccaccccgac | cttgtctttc | ccgaagggaa | tggctcctcc | ggcgcctcaa | cgcacagaaa |
| 42301 | acgcctgcct | ccagctgagc | gcttttgcca | gctttctcca | accagtgaat | aagggatgca |
| 42361 | ctatggactc | agaaggaaat | gtcatattta | agttctgcct | ctgatttctc | tgctcttagc |
| 42421 | tgatctaagg | tttttctaga | ctccttagtt | tccactcaca | gtgctgggta | atggagccct |
| 42481 | accgcactga | ttgagaattg | ccggccagtg | ggttggtaca | gagcaaattg | tccttgctaa |
| 42541 | caaccttttt | cctttgctgt | tggattccac | taagtggtga | gaggggtcag | ttttagattt |
| 42601 | ttgcaattcc | ccagcgtgga | gacaggccgg | ctctatacga | gaataagtga | taggcagagg |
| 42661 | aagaatttgg | cagtgttccc | gggttgattt | tattttggtc | tcttgaacac | attattcttc |
| 42721 | ctgatagaac | aaatccctgg | acttggtagg | acccagttcc | agggaaggga | agagtcctct |
| 42781 | accgcacccc | cagaacgccc | tgcagaggct | gtgagtgggc | aaagcccagt | ttcctggact |
| 42841 | cccacttagg | aggtgctgca | gaaataaggc | cttcagctgg | ggctccctga | gtcggattgg |
| 42901 | aaccgtggcc | catcagtaag | agtagactgg | atggtaactg | ctcgattcct | ttcatcaagc |
| 42961 | tctcatccct | ttgtcagaaa | tagatgaaat | cgattgctcc | cagaaggcca | tttgagccac |
| 43021 | taactccctc | aaagtacttc | agattcatgc | aatatggaga | ttcgggttgg | tagttcaaac |
| 43081 | tgcatcatat | ctagtcaagt | tttttgaaaa | catcaaaccc | ataaatttca | gaaaagcaat |
| 43141 | acaagctcca | tatttttagt | aggagctaac | ctagttttga | gatgtatgaa | tagatgaagt |
| 43201 | gaaactggaa | aaacattaaa | agttggaatt | ttaattaact | aatgtttaaa | ataagcctat |
| 43261 | tagtggttat | tctgtcaagt | gactgtgtca | tgcaagaggt | gtcatctggg | cctcattttt |
| 43321 | ggccatctcc | ttgatttgta | ctgacagaga | aggaaggaaa | taaacaaatg | aatcttcact |
| 43381 | ttctgattct | acctttttag | ccttttaaaa | gttaccttga | aaattcctca | aatattggag |
| 43441 | aaacgactac | caactaaaac | gttaacacaa | agttacaatg | tgaagttttt | gtgatacagt |
| 43501 | atatttacta | accaacaaga | tgaagaaaga | taatgctaaa | ttttgaatca | cactagattc |
| 43561 | aaaacatttg | ctagagtaga | ggttacagcc | atatttgaaa | actgtattat | ttttctgaga |
| 43621 | taataatatt | aaatgatttt | gccttcattg | gattactttt | aaattgggaa | aattaaaata |
| 43681 | attataacca | acagaaacac | tcttcaaatc | aatagagaca | tcattagaga | gaacatattc |
| 43741 | aaaatttgaa | aggactgttt | taaatagggc | aatctttaaa | atataatttg | aacaaccaga |
| 43801 | atagaaaact | ggctgcctct | taatatttaa | tcatagtttt | atgtaaatag | ctaaaatttt |
| 43861 | aaatcaataa | aaaataattt | ttaaaaatat | caaacaatgg | ccacaacagt | taagaattgt |
| 43921 | tgaatttatt | ctcaaaggat | gtaatttcat | tttgcatttt | tattagatga | ctctggcagt |
| 43981 | tgcctgtaga | atgggttatg | ggcggagcaa | gggaagaaat | gaagagacca | gttttgaagt |
| 44041 | aaccatagta | gaatctaaag | gagaacttca | acaaggatct | ggcggtggag | attggaaaag |
| 44101 | gatgctttcc | agatgtattg | agaaagtcca | actggacaga | tggtggtgga | taagccaatg |
| 44161 | aaggagaggg | tgttaccaaa | gattcaggcc | ctcatcactg | atgggtgcga | ttgccttcat |
| 44221 | gtaagcaaag | ggacactagc | aaaaggctaa | tttggaagga | agatcatgaa | tttgattggg |
| 44281 | gttaggtaca | gtttctggtg | tctttgaggc | aatcaaatga | gttaggaatt | cagaggagat |
| 44341 | gtttggattg | gagattttaa | gtttgtgagt | cactgacatg | tagatgatgg | caatcaaagt |
| 44401 | ggaaggaatg | gataaaattg | ctgaggaaat | ttcaggagac | ccaggatagg | aagttaagga |
| 44461 | ccctttaaag | cctggattgt | cacagtgagc | taacaaaata | ccttgagaat | gagcagctgg |
| 44521 | gtggtaggtg | gaaaaccagg | agagcctatt | gtcataaaag | tcaaggagag | ggggaatttc |
| 44581 | taaaaggaag | ctctggacaa | gagtggaaca | ccatcaagag | ttccactcgg | atgaggactg |
| 44641 | gaatgcaatt | ggacttagtg | gtatcatggt | tactgagaat | tgactcttcc | aagagtcatt |
| 44701 | cagtggagcg | atggaggaac | aggactagag | gtcagattgc | aatgggctga | agagtcaata |
| 44761 | gaaggtaaag | aaagagagag | aacacataaa | gataaaattc | tctctggaag | tttctactaa |
| 44821 | aggggaggag | acagacgata | gctctggaag | gaggagatgt | ggagggaggt | tgacttttta |
| 44881 | ttttatttta | tgtttgcttt | taagataggg | tacttgtgaa | cattcttgaa | tgccaatgga |
| 44941 | tagaatccag | tattggggga | tatgttgaga | ttaaactact | gaaaagataa | tccgggtgtt |
| 45001 | gattgcctgg | gaaggcagga | agggatacaa | tctacagccc | agatggacag | atttaccgta |
| 45061 | agttggaaga | gggacagctc | ctctgttata | gcagtgggga | aagaggagct | gatgggtgtg |
| 45121 | attacaagaa | ggtttgtcat | ttcagtgggt | gggtgtgggg | ggcaatttct | ctctactctg |
| 45181 | gttttctctg | tgaagtaagt | cgtacctttg | agtaagaatg | acagtggagg | agaggagccg |
| 45241 | aaagtgcctg | gagagcagaa | aaaaattgac | ggggacgact | aagctgacca | gagaagctgt |
| 45301 | tgttatcatg | ttattgtttt | gataatttcg | taagttaaaa | atttgagctg | ggagcacatt |
| 45361 | ttctatggaa | ggcattctcc | tagaaaaatt | ggctagtttt | tcatgaaact | caaaaaaatc |
| 45421 | tgcttaactc | acaaggaagt | agtaatataa | tatgaatggt | attctttcag | ctttagtgac |
| 45481 | tcaaagtagg | tgttctataa | atgttctttt | ttaaaaagaa | aattttccaa | ccattattta |
| 45541 | cagcacatta | aggaatgtgt | ctattgatgg | gtaggaaaag | tttcccccca | accaaataat |
| 45601 | tctttgtaat | aatgtcttca | cttcctccct | gccctttgtg | ctttatcctc | ctagagatag |
| 45661 | ccaaagaaga | atttaaagtt | atcatttatg | tcctgtacca | gacctccaca | gactggctta |
| 45721 | tttggataat | tcaagtgaaa | tacaaccaag | atgtttcttt | agttttgcga | agtctagact |
| 45781 | caacatcaat | attgcacaca | tttttcagat | atatatgtga | tgttttttga | attgttatac |
| 45841 | aatttcctgc | cttgatattt | cagtcaaatg | tattgggtag | gttatcagag | ggcgttgttt |
| 45901 | tcaaaaaaag | cctttttcct | tcaatttatt | ttgttaaagc | atcagactgc | caaagaatct |
| 45961 | taacttctat | gtaaggggtg | atacattaat | acctttttgg | ttgattttca | gctacagaat |
| 46021 | gcacagcctt | gatggacttg | ctttaatcca | cactaaagct | tatatgacat | gaggaagtca |
| 46081 | tgcagaacag | aacaggcctt | tctggaactt | ttatatttta | agaagtcttc | agcctgttag |
| 46141 | tacaacaggt | taccttttgg | caggcacatt | ttcctcctgc | tcgaatgcta | taaacacccc |
| 46201 | agcagggacg | ataaatagca | atgtagacag | caagggaaaa | tatttgctaa | aagatactcc |
| 46261 | tgcactttct | ctttttgatg | tttcccttgt | aagttttagt | tgaagaaact | ggaaaaaata |
| 46321 | gaaatgtaaa | agttgaaaca | gcaaaattat | aaagtgacag | gtgatacaac | aaaacatatc |
| 46381 | agtttgtact | caaaatgtaa | agcaataaag | caagcacaca | ttgagactct | ctaattagta |
| 46441 | taatttgcat | tcacaaagtt | caaagtggaa | cttgggtatt | gccattctct | tgcttctctc |
| 46501 | cctaacaagc | cacaggtagc | cgaaatgagg | cacattgaga | gttcaaattt | ggcaaaactc |
| 46561 | tgcagcaatt | cagtgacaga | ataaaacatg | ggaaatggat | gttttgaaat | tgagtctgta |
| 46621 | tcttaaagaa | agtgaaagta | aaactttata | ataaaataag | gctaaatcat | acacaattag |
| 46681 | tgtatccttt | tgaaaattag | tttgtgtttt | ataaggtcaa | atggcttctt | taaattcata |
| 46741 | ctgtgctatt | gtatctagcc | tggcatgttt | ctttactgta | gttctttttc | tgaattttta |
| 46801 | aacccctttg | tgatttcaca | gagttagtaa | ttatgttggg | atgttggggc | gtggactgga |
| 46861 | tgtagctaaa | catactcaga | tttgactcac | attcaaatgt | actcaaacat | tcaaatttaa |
| 46921 | agttgtctgc | caaagcaaac | ttctgtctca | ttgattttta | aaattctctg | gaccttggtt |
| 46981 | tgaaaatgtg | ttctttgggt | ttcaaaacag | ttatctgtct | tagatagatc | aactctgaca |
| 47041 | tctacatatt | tgacactagt | tctgattatg | tcaagtgcat | atcttttatt | atgtagataa |
| 47101 | aagacaacaa | cttgcatgtc | acgtaaaaga | ggaagcatct | cttcaggtcc | cgggtacatg |
| 47161 | agtgagtggt | gatcctggtt | tttcagttgc | atatcaagaa | acacaaccta | gatgagctta |
| 47221 | atgaaaaagg | agattagtgg | tcataacagg | gaaactttgg | agtgaacttg | gcttcaggca |
| 47281 | tagctggaac | caacggttca | gtggatgcca | atcagcaggg | tgtcttctgt | gttttcatct |
| 47341 | gagctggctt | ctttctcaag | caagaactcc | ccaggcagtg | tcaaagatag | taatgacagc |
| 47401 | tccgtagtta | aggggccctt | caaggtcatg | atcactgaga | aagtgttcag | gcagagggtg |
| 47461 | ctttctccct | cagagtatgt | aattatcatt | aataaaattc | tgattcactc | cagttggatg |
| 47521 | atgtacccat | ccctgggcca | gttagttgtc | aagggagacg | ggttactctt | aagcccaccc |
| 47581 | tggctgtggg | catgagaaca | catggtagac | agaatgcaga | caggccatct | ccaagaggaa |
| 47641 | aagggagtct | ctggattctg | tgatacaaat | aaagggaaaa | ggacgccagg | cagtcagcgg |
| 47701 | aaacaggtgc | ccaccactcg | gcagcccatt | ctcttggtta | tagttagttg | ttttcaagtg |
| 47761 | atagtacagt | tgactcttga | acaatatggg | ggctaggggt | cccgacccct | cccctgcaca |
| 47821 | gttgaaaatt | cacatataat | ttttgactcc | cctaaaactt | agctagtaat | agctcactgt |
| 47881 | tggtcagaag | ccttcccaat | aacatagtca | actaacccat | atcgtgtaca | tgtattatat |
| 47941 | gctgtgttct | tacaatgaag | taagctagag | aaaagaaaat | gttattaaga | aaatcagaag |
| 48001 | gaggagaaag | tacgtctact | attcgtgaag | tggaagtgga | ttatcgtaaa | ggtcttcatc |
| 48061 | ctcatcatct | gcataaggag | tagattgagg | aaggaggagg | aagaggaggg | gttggtcttg |
| 48121 | ctgtctcagg | agtggcagag | gcagaagaaa | atctgtaaag | aagtgaactt | gcagagttct |
| 48181 | aacctgtgtt | gttcaagggt | cagctgtaca | tgttcttagt | ttttgttttg | gttggtgtta |
| 48241 | gtgtcgtgtt | tgttttattt | tgctttgctt | tcagtttttt | atttgaatat | ccagctgtgg |
| 48301 | cagacagcat | gaatctatag | attttacttc | aaaatctgag | gccagtactg | tatgcagggc |
| 48361 | cagctgtcag | taaaattata | aagcaggaag | agcttttgtt | tgttttttga | gacagagtct |
| 48421 | cagtctgtcc | ccaagctgga | gtggagtggt | gcaatctcaa | gtcactgcaa | cctccacctc |
| 48481 | ccgagttcaa | gtgattctca | tgcctcagcc | tcccgagtag | ctgggattac | aggttcccac |
| 48541 | caccatgccc | agctaatttt | tgtattttta | gtagagatgg | gggtctcacc | atgttggcca |
| 48601 | ggctggtttc | gaactcctga | cctcaagtga | cccgcctgcc | tcagcctccc | agagtgctag |
| 48661 | gattacaggc | atgaaccgct | gtatacggcc | aagaggagtt | catttttaac | tccatctcct |
| 48721 | tttgggtgta | tgtccctaat | ttaattcgct | cttccctgtc | ctctcacaag | gacttccaaa |
| 48781 | gaaatagagc | agtggcagaa | cacaatactt | aactcataga | atggtgagat | tgcaggcacc |
| 48841 | actgttagcc | aggtgaggaa | ggtttttgga | ggcaaggaac | cttccaaaat | atcttcataa |
| 48901 | ggatgtaagg | aacctatgca | tacttggagt | tggagtgtac | tgctgtgttc | agacatttaa |
| 48961 | agctgccttt | gtaaaggaaa | tgagattata | tttactctgg | ggagattgcc | atgaagacag |
| 49021 | tgcttgagaa | gcacctttaa | aatatgaagt | tttcctcaac | aaatgcaaca | tttttaaggg |
| 49081 | gctacgactg | gtggactaca | ggtgtgaaaa | gtgtgcagta | taacagcaat | agcatcataa |
| 49141 | atggaaaagc | ttggaacaga | ctgttcttct | gcagagcata | tcttgtgtgg | tgtgtttttt |
| 49201 | tcgaggtatg | ttaagagtct | tggaacaaaa | gaattacttg | actatattga | gatgtatctg |
| 49261 | gaacaggccc | acacacgtct | gtggtgccag | tttattcttt | tacaaaatgt | tacctttaaa |
| 49321 | aaaaattttt | aaatacacac | ttaaatgggg | atgggagcac | tgtcagggag | agtctcacct |
| 49381 | atgaaagcaa | aaagcacagc | ttaatttcag | agcactgcac | tgggagtcca | gtctccgctc |
| 49441 | tccacaactc | tcagagaaat | tctgaacaca | gcactttgtc | atattttcca | ctaatttgtt |
| 49501 | cccaaggtct | ctctccggag | agcgtctaca | aactcaaccc | ctgccctgaa | ggggctgtgg |
| 49561 | aggaagtggg | caaggttgtc | tacagtgtga | ctttcttggg | atacttcttc | atcctggggg |
| 49621 | aaggcctaat | gtctaagtga | ccacctgtga | gcaggtgttt | ctctcacagg | gatccgtttc |
| 49681 | tacgagcaga | cacccttgtg | gctcttgttt | gatctgtgtt | cagtttattc | ctaccaagat |
| 49741 | agctactctc | taggaaagcc | tggatcggtt | tcctctctag | gagaccccag | aaggaggttg |
| 49801 | ggttcaggta | tgtcaggcag | gggagacaca | aaggaggcaa | ctcaacaaag | cacatgaaac |
| 49861 | agcagaggat | gtgtattact | cacaggtccc | agagaggaag | ggagcaggac | tcatagggcc |
| 49921 | agctggaagt | ggggaactgt | ctgagacaca | cacatgcaac | cagggagtgg | ggagcaagag |
| 49981 | agagagttgt | aggggggacc | tgtgggccaa | agcccttact | agggtctagg | gcattaccca |
| 50041 | agtaggcttc | ccatgggaag | ttctaatcgg | tgggtgagct | ctgtggagac | tgcaactgag |
| 50101 | agggggtcac | tgcagcatat | ctgtgtgttc | tatgttggtg | ggggagttgt | tggggcagtc |
| 50161 | agtggggcca | gtcaagtagg | ttgtatccag | ctctccctta | gggaggtggt | caccagcaag |
| 50221 | tggttgtcaa | ccaccttgag | ttactgggag | gaagcagaga | gcagaagact | atgtcaaaga |
| 50281 | tgactaagcc | ctgctccccg | tatgagaaag | tcaagcctag | attcaggacc | gatgccagag |
| 50341 | caacataaaa | ctatagaact | cactacatgc | ttcatgcagc | tcggtttcca | gtgtttccat |
| 50401 | ctgtaagaga | caagtattga | attaagtgat | gtatcaggac | cttatcagtt | gccaaattga |
| 50461 | atcattctac | tgtttttttc | agcagctttg | ataaaacatt | agtgcaggct | tgtcattacc |
| 50521 | caatactact | ggaggccttg | aaagtgtgga | gtgctgtact | aggatggcac | atgacgtgac |
| 50581 | ttatgctgac | attgactcct | atgctgacat | tgactccttt | gcacagctga | tatttgaaac |
| 50641 | gctcttggta | tattttcttt | ctgaattgtt | tgtgttccta | acatgggcca | tgaatctctg |
| 50701 | aagtggacat | ggataccatg | tcctatgtct | acctgtacat | attgtcagct | tgcaataaga |
| 50761 | aatatgataa | taaaatgaag | ttctatcaga | ggagtggcag | gggatgatca | aaccacaaag |
| 50821 | aaaacaaagc | aaggaaacag | cacaacgcaa | acacagacac | ccctcaccaa | aactcttctc |
| 50881 | tgcctgattg | ctctaaaaca | caaggcaagt | caatattcct | gttcagctga | gaacaagagt |
| 50941 | gtttacttca | tggaagatct | ttgagagtta | gctaaattaa | aaatttaagg | aaaaaggctt |
| 51001 | atcaagaaaa | tatgagtcaa | aatgtggctg | ttccttttta | tgtttctccg | atttcgtgct |
| 51061 | attttgctac | tggaaatttt | ctgcctctct | ggatactctt | gcaaatggga | acagatacgc |
| 51121 | ctagcctggg | ttcctgctcc | atcattcccc | cagttctagg | agcaggagcg | gagcaccact |
| 51181 | ggtgctaggt | caagtggaag | acaaatgatg | ccacattttt | gctgtggatt | ttcttttatg |
| 51241 | tcgtctctag | cttctggacc | agcggtatat | tatttttcag | agtctttttc | agatcaattc |
| 51301 | tgagcttaat | tgctgacttg | cttgtgaggt | tggaagctct | acctacagtg | taccatgctt |
| 51361 | ttgatagaga | aggtttctgg | gcagtttttg | gtttttcata | gaggtattgg | ataggctcat |
| 51421 | actgattaag | ctgtggctcc | agcaaacatg | ctcaatatta | tgaagtccag | gaggtaggtt |
| 51481 | ttgttgctaa | cataaaaata | ttcttcagct | ccacgctggc | ccaaagcagg | agggggaagt |
| 51541 | tggagaggaa | cttcttcatc | catcaaacaa | agtgtatatt | tgttgccgtt | ttgacaatca |
| 51601 | catcccttag | taaaagatgt | acttgaaaaa | caatataaat | aaaataaagt | ttcataaagg |
| 51661 | ctcccaaaca | ttcttccttt | ccccctgagg | ctataatgac | tatcctggtt | ttaaaatccc |
| 51721 | ttttcttatt | ccttctgagt | tttcccattt | cgtgctccct | gggagcatct | ctgtaaaata |
| 51781 | agcctgtgga | aaatgggtaa | tgaggaaatc | gttgccttaa | ttgaaggtcg | catctacaaa |
| 51841 | ggtccaggta | ggtgcacttt | ggctgatgga | aaaatccccc | agagtgacat | cctgctgccc |
| 51901 | tgaaatagag | gctcggttgt | catttgttag | aacaacggtg | gcgcctggtt | ggcatctcga |
| 51961 | gtgttgtctt | tgttaatctc | acaaccaggt | gatttccatt | actgacgaca | ccccacatgg |
| 52021 | gaagggggca | aggaagggcc | cccaaggact | cccataatta | agcacaaaca | cttttacagt |
| 52081 | attaagtcaa | agccgaatgc | ctcccaaatc | ctcttggtgt | tgagttcgtg | tgccctacac |
| 52141 | aatggtgacc | tttaatatgt | ccctttcaga | tccagctaga | ttagtcctgt | gctctggcct |
| 52201 | tgatattttt | tgttttgttt | tctagtgcta | ttcctttaaa | agaagaacta | tgagagtgat |
| 52261 | ttttttaatc | tgtttgctta | cttttctttt | ctttttttgg | cttattgtca | acatgttgtg |
| 52321 | ttgacacact | ttttagccta | aaacagagag | aggaccttct | aaatacaaaa | gataggatgc |
| 52381 | tttcaacaac | tcgattccct | aaagcactgg | agaattctgc | gatctgttag | acttcaatgt |
| 52441 | aagcatttta | tagaggttcc | catgacagga | ttattagtca | gcagtttcac | ttaaaggtct |
| 52501 | tttttatttt | tttggccagt | agactttctc | cttctacagc | tgcaactgta | ttaaatatac |
| 52561 | atccccaata | atttagctcc | tcttctttct | ctggtggaat | attttcagtt | atgtaatttc |
| 52621 | agctcctgct | gcttatttgg | ggtgattatt | attatatgtg | gtccctagcc | taggaatgaa |
| 52681 | ttctgctgta | gataccagtt | tttaaattag | ttagaattct | gcctggttcc | ccacaggaaa |
| 52741 | aatgtcataa | aagatgggcc | acaagacagt | ccaccaagcc | attttaaact | cccacatagc |
| 52801 | tgaactttca | tcattggcag | ctctgagttg | tgaggtctct | tgtgacactg | gccctggtta |
| 52861 | tttttaagag | gggcaagaaa | ggtaaatcaa | gagggtagtg | atagaagcag | gctagaaaaa |
| 52921 | gccaggcttt | ggcagctggg | ctgctctcac | gggattatga | ggggccatat | tctaagtagc |
| 52981 | aggaattgaa | agtggtgaat | ggattcaggt | cctgaccatg | catgaggctc | ctgtcaggag |
| 53041 | gtgccataag | agctgagcac | atcaatttga | agaagaggct | catagagagt | tgggtgtcct |
| 53101 | tcatgggacc | aggatggcgt | tgaggggtaa | aggtggacag | caattgcttc | ttatttgtac |
| 53161 | acagcttatt | tgggagttgg | agctcatctt | attaatgaag | gtaaaattat | gacttgaaaa |
| 53221 | ccaaccatat | ttcactacta | gagtatcatt | atttgatgct | ttcaaagtct | ccatgtgctt |
| 53281 | gcaccttatt | gatcttgtca | ccagttctcc | taaattccaa | tctggatgca | taagaaatat |
| 53341 | caaattgccg | aggtgggcgg | atcacaaggt | caggagattg | agaccatcct | ggctgatatg |
| 53401 | gtgaaacccc | atctttactg | aaaatacaaa | aaattagcca | agcgtggtgg | tgcacacatg |
| 53461 | tagtcccagc | tacttgggag | gctgaggcag | gggaattgct | tgaacccggg | aggcggagat |
| 53521 | tacagtaagc | caagatcgcg | ccactgcact | ccagcctggc | aatagagcaa | gactccgtct |
| 53581 | cgaaaaaaag | aaagaaatat | caaagtggtt | ttggtataaa | gagctagcct | tcccgttggc |
| 53641 | ctcaaggata | cgttagagat | aacagcaagc | tgtctaaata | tttgcagact | acaacattca |
| 53701 | aagataaggc | aagaggacca | atcttaaaga | ttttacaaaa | taattgcaac | aagtgcatgc |
| 53761 | atctgttgtt | tctcaaatca | tgttttaatg | ttttagctat | taggtgaaaa | tggctatgta |
| 53821 | caatgtgcta | tttttaaatt | gtcaagagta | gatttggaag | gttattatca | atatcctaca |
| 53881 | cttttccttt | cgtgactcaa | taataatttt | tgcactttca | tttagcctta | aaaaatagaa |
| 53941 | caataatgca | tccaagtata | aggtcaattg | caaagtgacc | cctgtagttt | ttgcttcaaa |
| 54001 | actattatat | tgtgctattt | ataatatcaa | attgccactg | tctagaggca | aaatgatcat |
| 54061 | ttcaaaatct | ttgtaacaat | ctagagaaat | atttacaaat | attagccaca | attctagtgt |
| 54121 | aatttttctg | atgtgtttct | attgctgaca | attgttttgt | aaaatcttga | tgattggtcc |
| 54181 | tcttgtcttt | gaatgtcgta | agtctggaac | tatttagact | gcttgctgtt | attgctgata |
| 54241 | tatccttgag | accgtcagaa | aacctagctc | tttttcccaa | caccaatttg | atatttctta |
| 54301 | tatatgcagg | ttggaattta | gaagagctgg | gtgacatgat | caataagtga | ttttgagaat |
| 54361 | tgctcagaac | ttcattttat | cttttttttt | tttctcaaac | aacctttaaa | atcagaaact |
| 54421 | ctggcatagc | actctccaac | tgtgccacag | aaggacataa | ggatgttggt | gctttttcct |
| 54481 | gtctattttt | gtcccctacc | aaatcacagc | cctggaaggg | agctttagaa | gtcacaaatc |
| 54541 | tgttctgttg | ccttcatgct | cagtatcaca | gaagagggat | aatcaaccct | cttctaaaag |
| 54601 | gctagccagt | ggaaaggatt | tcaaaatcag | ccaacaagta | tttttgctta | ctgtgtacat |
| 54661 | ggaacagtat | ggaaaaaaga | atatggaaaa | ctattagaca | ttattcatac | ccccaagaat |
| 54721 | cttgcacttc | atttggaaag | aatagatacc | tatatgtaca | caaaaaccac | catcaaaact |
| 54781 | tctgaatttg | gaacaggtct | tgaagaccta | actttctgac | tgttgtaaat | atgcactctt |
| 54841 | gacatcctgc | catttgatcc | tatcagttca | tcactgggaa | attttaattg | caaataaatt |
| 54901 | ctatccactt | ctaaattcta | aaggctttaa | tgcatgacag | gtctatagaa | gttaaatgac |
| 54961 | cgtagaaatt | gtcagcctgg | atagaaattt | tggctttacc | aatcattaat | agtgtggcct |
| 55021 | caggcaagtt | acttaatgtt | tttgcctttc | aatttcgtat | cataaagtgg | ggaaaaatac |
| 55081 | tagtatcttc | ctcatagggt | tgggagagtt | aggtgttgaa | ttttcagaat | agcccctgat |
| 55141 | agagggtaag | tccacatcac | attaaatggt | atttgtacaa | ctgtcataca | aatatttgac |
| 55201 | tcaactttta | atgttctgtt | ttccctgctc | cagctacccg | cttttttttt | taaaggtaaa |
| 55261 | acctcttcat | ctgtgtgatt | aatgatagaa | ctattccact | acggtcacag | aatatgattt |
| 55321 | ataactgact | aggtcctcct | tgttggtttt | taatgccacc | cttactacaa | atatggtatg |
| 55381 | aatagtaaaa | aaaagtgggc | caattattat | gttctaaata | gtatactact | aacactgcat |
| 55441 | cccaagcttg | caaaaatgtt | tcaggaaaat | ttttcaggtt | tcaggaggcc | acctgagtta |
| 55501 | gacaatggag | gccttaagtc | tattttactg | attgtgtttt | tcagaagctt | gagtttgttc |
| 55561 | tggactttag | cttccaattg | tgtgaaatgc | ttgaattgac | ccaagactat | gttaccgtca |
| 55621 | ttctgtcctg | tgtgttgttc | tttctcaaat | ccatggatat | tggtgaagtc | ctagtgtttc |
| 55681 | atattaattt | ctttggcaga | tattcagttt | aattctttgc | tgggatagtg | tgcatgggtg |
| 55741 | tgcatgtgtg | tgcatgcatg | cacatgtgta | cacgtgtgta | cagtatcatg | cttttgttcc |
| 55801 | actcatagtg | ggataaatat | attccttaat | aacctggtct | gatattcctc | ctggagtctt |
| 55861 | actgttccag | cctaccctgg | ccacactctt | gctttaggaa | ctctcacatt | tatccttcta |
| 55921 | agttgcttac | acaccaacct | cctcattttt | caaagcccag | ctgaaatatc | ttctcttctg |
| 55981 | tgaagacttc | ctagatcccc | tggccaggat | ttgtccccta | tcctctctct | gctccccagc |
| 56041 | attttgttaa | ttctccttct | cagttggcat | tttggttatt | gatgtagctt | ctgcttcctt |
| 56101 | tatgggactg | tagaacactc | tagaagaaga | gctttatctt | attcatcttt | gtatcctagg |
| 56161 | gcctagcaag | tggccatata | aaatttgttc | agctgaataa | agaaataatt | ttaaaaaaag |
| 56221 | aaataatgaa | aaggttcaca | tgggggtgta | ttttctttct | gatgttatca | tcctaattat |
| 56281 | tagaagacat | ggagggtctg | ggaggagaac | tgatttctaa | tgaacatcca | tttggtgcca |
| 56341 | tatcctactc | aatgtggttt | ataaattctc | tcatttgatc | tttgcaggga | ctttataaga |
| 56401 | ttagtattat | tatccccact | tatatatgag | taaaattaag | ttcagatagt | ttgaggcaca |
| 56461 | ggcagagtct | aatttataaa | ttccaggtct | tggaagtcaa | tgaaaactag | aatgaggact |
| 56521 | caaatttact | tgatgttacc | ctaaagccat | agacatattt | aatgacaccg | aagtacatga |
| 56581 | agtggtttga | cttctatgga | aaaaagctgt | ataaatatga | taatgccatt | ttctcattca |
| 56641 | aaaacccttc | aagagcattg | tttttgcaaa | ccaactaact | acattccaat | ttttcagcct |
| 56701 | ggcacttaag | agccttgata | ttcgtttctg | ttcttcaatc | ccattgcttc | taccttgatg |
| 56761 | ctctgtccag | ttggactgaa | ctctctggat | atctgcagat | ttctaccttt | ctacctttgc |
| 56821 | tcactctctt | tctcccacct | ggaatgcact | ttgtcaggga | tatacttgca | atattaatta |
| 56881 | gcttacttag | ccttatcata | atcaaataca | tggttctctt | aagcttttgg | aaaaattaaa |
| 56941 | ttctaagtgg | accaccattg | caaactctat | ggctctctgg | tggttcagga | atctcaggct |
| 57001 | agaaaaaatt | ctcttaaagg | aaaaaatatt | catattggta | aattttttta | aaaaaattta |
| 57061 | agtatagtac | ctttcattaa | ttccttggaa | acaatgctaa | aacttaagaa | cagagaaagt |
| 57121 | ttgttgttgt | tttgcttttt | ttttttaata | aaagaatcta | gcctaagaca | ccattctaaa |
| 57181 | caaatgtgtc | agactcatca | acctcctaat | caaaactttc | actcaactaa | ttctttcact |
| 57241 | tacctcctgc | ccactgcagt | tatctacagc | cctggctttg | gcaaaatatc | aggattgccc |
| 57301 | tattcatctg | cctattggat | ttgatccaga | attagtcaca | actttgttta | attctccagt |
| 57361 | cttcagtcaa | tctctggtcc | agcatgtata | ggacagtaat | tacacagtca | cacaatgcag |
| 57421 | cataatgtat | acttctcagc | acattcaact | ctgtgttttt | tagggtaggg | ggtgggagag |
| 57481 | agtatctcta | aagatgacta | aagcacagct | gttgggtgga | tcctccctac | aaatgtaaac |
| 57541 | caaaggattg | gggctgacct | cagagcgact | cccgagccgg | cactctctgg | gataatttcc |
| 57601 | tccatccctc | ccaatcctcc | cagaagggag | tctacttttg | ttcacatatt | tttatagaag |
| 57661 | gttaagtctt | tcatgggttt | tgtataaaag | ttgaaactgg | gttttgattc | cttgagatga |
| 57721 | gcagtggtaa | acatgtgtgg | cattcggaga | aacagcacct | tagaattaca | cctgttgttt |
| 57781 | gtgcggtgtg | gctctcttcc | tatggaaatg | tgtgaactct | cctaggagaa | agaagagaaa |
| 57841 | tatttaaaaa | atcaaatgcc | tcagggcaca | gagggttatt | gtgctcacaa | tctcttatct |
| 57901 | gcgatcttaa | aattcaaata | gaactgaaaa | attcaaatag | aactgacatc | tgcatttatt |
| 57961 | tatttattga | ccactcattt | ggagaccaaa | cctgaccaga | tctaaacttc | cttggcaaga |
| 58021 | tgacttgacc | ttacttattg | caagcctgtg | aatagtcttt | ccttatccca | gttagtgtga |
| 58081 | aaatccatgg | tgttgctaca | gaaatattca | tgtgtttgat | tagaggatgc | tgccttgaat |
| 58141 | ctcacagggg | ttatgtaaca | tagggcatgc | atatcctatt | tttttgtgac | attggaaaat |
| 58201 | ttctgaatgg | tgaaagtgcc | tggtcaaagg | gattttgaat | aactgattat | gacctataat |
| 58261 | gcaagagaaa | caaaaatcca | gcaaaatcaa | aatcagaatc | ttggttttca | acatccctct |
| 58321 | ctctttatac | cctatcatat | tcccccaatc | atatactcta | gtatgccaca | gcagcaattc |
| 58381 | ttgtacatta | tcaagatctc | aaatctaaag | atcttgattg | tccacacttt | cttccctcct |
| 58441 | tattctccac | ccatcattac | aatcgcttct | ttcatttgct | tacccctctt | attcttcatt |
| 58501 | gcacactcct | ggcgtatctc | catccctgtt | tccaccaatg | atccgtctat | tccgtgctgt |
| 58561 | cgccgaagca | gctgaacatt | gttaggggga | agaatcacat | aattgtgctg | actggtctca |
| 58621 | tttaaattca | taaccacaac | tgaactggac | actgttgtag | tgcagcccta | ctttatttcc |
| 58681 | caggttgttg | catgcttatg | tgacaatgtc | acaccttctt | atgacctttt | caaacgtctc |
| 58741 | tattgactgt | tctatcttgc | cccgcttttt | caactcatac | gtcattgaaa | aaatataagc |
| 58801 | agaaagtgaa | aatgaattaa | ctactctttc | tttcaccaaa | tcaaccaact | taattgcatc |
| 58861 | tgtacccata | tagtattcct | tctctcctat | tttgatagac | aaaatatccc | tggtcctagc |
| 58921 | taagccagcc | cctccacttt | tggcacctcc | tctgcctttc | tcaagaactt | cagtcctaca |
| 58981 | tttattcttt | ctctctctct | ctttccttcc | cacatcatca | gtttattcac | tgactccttt |
| 59041 | ttatctgcac | ccaataagca | gtagtggcca | ggtgcagtga | gtcatacctg | taatttcagc |
| 59101 | acttcagaag | gctgaggcag | gaggattccc | ggaggcctgg | agtttgcgac | cagcctgggc |
| 59161 | aacatagctc | atctttaaaa | acaaaataaa | acaacgttag | ctgggcatag | tggtgcacac |
| 59221 | ctgtagtctc | agctacacag | aaggctgagg | agaaaggatt | tcttgagccc | aggagttcaa |
| 59281 | ggctgccgtg | agctgtgatg | gttccactgc | actccagcct | gggtgacaga | gtgagaccct |
| 59341 | gtctctaaaa | caaataaaca | tgctgtagta | ttctctgtat | cccctccagc | tcttgtgctc |
| 59401 | ttctctggtt | tttatcacga | tacaacttag | gacataagtt | agcatcactc | cctccacttt |
| 59461 | ctctccttac | ccagttccat | caggtgatca | ctgccatctt | ttcactgatg | ttagtcttgt |
| 59521 | taaggtcccc | agagacctgc | aggtggtcat | tggcagtggt | cacttctctg | gcctcatcca |
| 59581 | agcctttcat | ttgtcccgca | tttgacctct | gtcttttaga | accattttct | tctcttggct |
| 59641 | tctgtgacaa | cacaatctcc | tgatgttttt | tcttcctgac | cttcttctca | catcgtttgc |
| 59701 | tgcctgtcca | acctctatta | gctggggtat | cccaaagctc | agtgctatac | ccctttctac |
| 59761 | tccttatttc | actaggtgat | cttattcttt | tccatggctt | tttaagtaag | gttccaaaag |
| 59821 | tacaccccca | gccttgggct | tgagctcggg | attcatatat | ctaattgcca | gtgtggattc |
| 59881 | atctatctaa | ttgccagtgt | aactctccac | ttgaaagact | agtaggcact | tcaaacttag |
| 59941 | cacgccaaac | acagaactct | tgacactcac | cacctaaact | gttccccctc | tagtaccgtc |
| 60001 | catcttagtg | agcacagcca | tcaagtcact | caaggcagaa | tctaactccc | tccttccctt |
| 60061 | aactgccaca | tgtaatccag | caacaggtct | cacctttgcc | acgtgtaaaa | tacaccctga |
| 60121 | gtttattcct | ttctctctgt | tttcctagtt | taagccacca | gtatctccca | cctgaactgt |
| 60181 | tatggtacct | tcctgcacgt | cttcttctgt | actctcttga | ttttttataa | ttgactctac |
| 60241 | ataacaacaa | atactgtatt | taatcttgca | catgtatttc | tatgtcccat | ttaacttagg |
| 60301 | ataattttga | actctttgtg | ttggctcaca | gggctctcat | gatccaaacc | ccatctcccc |
| 60361 | atctcccttt | cctgccctcc | ttggcatcct | ctctcccact | acctcccagc | catgctgcct |
| 60421 | tcttcctctt | ctttatacca | gccaagcgca | ttgccacttc | agggctttgg | aacttcctct |
| 60481 | tacctccccc | ctcgtagatc | tctacaagac | tcattcccct | ttggcactca | agtttcaaat |
| 60541 | taagtcaccc | ctgcagtcac | tctctgtcac | atccctctgt | gtgacagcaa | ttagagttct |
| 60601 | tgttggcact | tctccctcag | tgctcactca | gttagtcaac | aagttttatt | tttctccatt |
| 60661 | cctctctttt | cctactgcca | acaccctaat | agaagccacc | ttcctcactt | gtctggactg |
| 60721 | tagtagtagt | agcccctcag | ccatctcccc | acatttgtac | ttattccctt | ctaacctgac |
| 60781 | acttggtaac | acagctgcct | tacttagatg | actgctgtga | cttctcattg | ctcttaaggt |
| 60841 | gaatttttat | ttttattttt | attttctgag | acggagtctc | actctgtcgc | caggctggag |
| 60901 | tgcagtggcg | tgaccttggc | tcactgcagc | ctcctgctcc | tggggtcaag | tgattcttct |
| 60961 | gcttcagcct | cccgagtagc | tggctctaca | ggcgtgtgcc | accaagccca | gctaattttt |
| 61021 | gtatttttag | tagagacagg | tttcaccatg | ttagccagga | tggtctcgat | ttcttgacct |
| 61081 | cgtgatccac | ccgccttggc | ctcccaaagt | actgggatta | caggcatgag | ccactgcacc |
| 61141 | tggccgaggg | ttaatttttg | aatccttaac | aggagtctaa | ttctctgaat | gggctggctc |
| 61201 | tgacgtttct | ctttagctgc | accagaatat | gtaagcattc | gttcagttcc | ttacatgatt |
| 61261 | cacagcccat | ttcaacgaag | cacatgcttt | ttccttgact | ggtaatacca | ctttacctgc |
| 61321 | tcccttttgt | cttcttaact | catgcccatc | cttcaagtct | gagctaagga | atcattttct |
| 61381 | caagaaaacc | catcctgacc | acctcaaaac | atctggattt | tccccttctg | ccacataaca |
| 61441 | tcatagcatc | ctgtactgct | cattcacagt | acatatcagg | atggtattta | attcattcac |
| 61501 | ctttaatgcc | atcttcctcg | ctagaatgtt | tcatcagcta | taatgtcatg | gtaaatggcg |
| 61561 | ctggggccag | attgcccagg | ttctaaaccc | taattccttt | ttcttacata | gctgtatgac |
| 61621 | cttatttagg | ctttcttgat | gtaaaattaa | ggtaattatg | gtacccacca | tgtagatttg |
| 61681 | ttttgaggtt | taaatgagtt | aataaacata | aagcatctaa | tatagtgcct | ggcagataat |
| 61741 | aaacactcaa | taaatcatca | tgattgtcat | tactcttatg | agctctggga | ttgcatctgt |
| 61801 | ctcattcatc | atggcacatc | caacaagtat | ttgttgaata | cataaataat | atttttcatg |
| 61861 | agccaattaa | agttagaagt | aatattatta | aatgcctaga | tgtacgctag | gctaggggat |
| 61921 | atcaaaatta | ataaaacatc | gttttcacaa | tcaaggagtt | tttaactcag | tgaataagag |
| 61981 | tgagatacat | aaatgaggtc | tgctcaaatg | gtgtagttac | acatctttgt | gccctgggca |
| 62041 | ttttcctagg | agcctgatac | ggtttggcta | tgtccccacc | caaatctcat | cttgaattgt |
| 62101 | agctcccata | atttccacgt | gttgtgggag | ggacctggtg | ggatataatt | gaatcatcgg |
| 62161 | ggcagtttcc | tccatactgt | tctcatggta | gagaataagt | ctcatgagat | ctgatggttt |
| 62221 | tataagggga | aacctctttc | acttggttct | cattctgtct | tccctgtcat | catgtaagaa |
| 62281 | gtgagggttt | gtttgttttt | ttttgacaga | gtcttactgt | gtcacccagg | ctgaagtgca |
| 62341 | gtggcacaat | ctcggctcac | tgatccccag | ttcaagtgat | tctcatgcct | cagcctcccg |
| 62401 | agtagctggg | attataggtg | cgcaccatca | catctggcta | attttttttt | gtatttttag |
| 62461 | tagagccagg | gtttcaccat | gttgcccagg | ctggtctcga | actcctgagc | tcaggcaatc |
| 62521 | cgcctgcctc | aggctcccaa | agtgctggga | ttacaggcat | gagccaccgt | gcccagtgat |
| 62581 | gtgacttttt | cttccttgcc | ttctgccatg | attgtgaggc | ctcccaagcc | acatggaact |
| 62641 | gtgagtccat | taaacctctt | tttctttata | aattatccag | actttggtat | gtctctatga |
| 62701 | gcagcgagaa | aatggactaa | tacagagcct | accccatagc | ttgtgtacaa | tgtgtacaca |
| 62761 | ccaaaccgtg | acctgcctag | ctgtctccta | gaggaaagaa | ggggcactct | agagagaaca |
| 62821 | gaaaacagcc | tgaggaagtg | gcaatgaatt | ttgccttgaa | gggccggtaa | gatttcagca |
| 62881 | ggtacaattc | aggaaatgga | gaggtaggag | gcattccagg | aaggacacaa | gtaaaggcaa |
| 62941 | gaagccagga | aaatgaatgg | agagcatgaa | ccgtctgatg | gtggagcagt | ggttctcaaa |
| 63001 | gcatggtcat | gggcccagca | gcatcagcat | ctggcctgat | gattgttaga | gatgaaaatc |
| 63061 | tttggacccc | tgaccagatt | tactgaatca | taaaatccta | gtaatctgtg | tcttaacaat |
| 63121 | cctctggttg | attctgttgc | tcaccgaagt | ttgagaacca | ctgggctagg | gcactgtgtt |
| 63181 | ctcactgggg | acccacgaac | ctccagagag | ttgcagaggg | attcatgtgg | tgacaatgac |
| 63241 | agagcaagtc | aagagtcaac | ccccccacca | ccgcccctca | cccaataggc | aagggcaatc |
| 63301 | gtgtgtgggg | tcagagtcag | tgctgttgtg | acttggtgga | ataccactgg | tttcaacctt |
| 63361 | catagaattt | gggtggcttt | ttgttgttgt | tttgttattg | ttgttgttgt | ggtttggttt |
| 63421 | ggttttttga | gacagggttt | cactctgtca | ctcaggttgg | agtgcagtgg | catgatcatg |
| 63481 | gctcactgca | gccatgggcc | cctgggctca | agcaatcctc | ccacctcagc | tttctctata |
| 63541 | gctgggacta | ccggtgagag | ccaccatgcc | tggctaactt | ttttccttat | tctatagagg |
| 63601 | cgggatctca | ccatgttagc | caggctggtc | tcagactcct | ggagttttta | attgattttt |
| 63661 | tttcagctac | atattggggc | tgaaaaggtg | gaatttttaa | aaacacaatg | taaggaaaac |
| 63721 | tagtgttaga | atataagctg | ttgagaagca | aatgatagta | atatttctgc | aaatttagct |
| 63781 | caagataatt | ttcacaagtc | agatccacaa | gtcagatggc | aaaattctta | taaagcttat |
| 63841 | caagatgtct | caaatgctta | aaaaataaac | agaaaaaaga | gaaaaatata | gtaataatca |
| 63901 | tatctagttt | ggctttcgtc | taataaaaat | gtgttatttg | gaaagtgctt | gtaaatagcc |
| 63961 | taaaaatttc | tttcatatca | tctttagaac | ttaacaatta | taaaaatgag | ccaattgatt |
| 64021 | tttacattac | aaaatgcaga | cattctaaat | atcagctact | aaagtagtca | tatcttgatg |
| 64081 | atgtaacatg | taattaaatt | ttaatctaca | tttttaatat | acaatagtat | attaaatcac |
| 64141 | atacagtgtc | attttaaaat | ttataactca | accatccttt | tttcttacat | aattaaagta |
| 64201 | aatcaaacag | tatgtgtcat | ataataaatg | ggaaaaagct | atgattttta | acacatggaa |
| 64261 | cagatattat | agaaattttt | attaaatgaa | ttttgtaata | tggtcttaaa | taatgcactg |
| 64321 | atctaaagaa | atttaaaacc | cttttcaact | taaaattact | ttgcttcata | ccatggaaaa |
| 64381 | ttttaaagga | aattgttggt | gaattattat | gaaaagggtt | catgagtcac | agagagagga |
| 64441 | aaatgctgga | ctagtgcact | gggtacctgg | atctgggtaa | tgggggagtg | tatcagtctg |
| 64501 | ttttcacgct | gctgataaag | acatacctgt | gactgggtaa | tttataagga | aaaagaggtt |
| 64561 | taatggactc | acagttccac | atggctgggg | aggcctctca | atcgtggcag | aaggtgaaag |
| 64621 | gcatgtcttg | tatggtggca | gacaagagaa | gagagcttgt | gcaggggaac | tcccctttat |
| 64681 | aaaaccatca | gatctcttga | gacttattca | ctatcacgag | aacagcatgg | gaaagacctg |
| 64741 | cccccatgat | tcaattatct | tctaccaggt | ccctcccaca | gcatgtggga | attatggagc |
| 64801 | tacagttcaa | gatgagattt | gggtggggac | acagtgaaac | catatcagga | gatagaggtc |
| 64861 | aaggagagtt | actgcagaaa | ggtgaggaag | aaaccagtcc | aagagatctc | caggttgtgg |
| 64921 | gaagttttga | aaccctaggc | ataggtaggg | cttacttttg | gaagagggat | catttcctct |
| 64981 | gagctaaagg | ggaacgggta | caactttgaa | acgtttctta | aaatactgta | cgtgccagaa |
| 65041 | gtcagaatgc | tcatgaagtt | ctcaaagtcc | ctatctagaa | aattctggaa | caattgctct |
| 65101 | attcagtgaa | agttgcctgt | gttctggata | ccatcacctc | aagtctcctc | tggtacgttg |
| 65161 | ttccaccagt | tattgcacct | ctcctatatc | ttccatcttt | ccttttccat | tcacctttca |
| 65221 | ttagcaaatg | gatgaacaca | cattttaaac | ccgtattaaa | atgggtttac | tcaggcaact |
| 65281 | gtaacaaaat | atcataggct | gggtggctga | aataacatac | atttattttc | tctccattct |
| 65341 | gaggactaga | aggctaagat | taagggtttg | tgtctagcaa | ggcctcactt | gctgtcttgt |
| 65401 | agactgaagc | cttcttgctg | agtcctcaca | tggcctcaca | tgtgagggtc | cagatgagag |
| 65461 | agatagccaa | tgtcctttcc | tcttcttata | aggatatgtt | agggccccac | ccttaagacc |
| 65521 | tcatttaatc | ttcttttttt | ttttttgagt | cggagtctct | ctctcgccca | ggctggagtg |
| 65581 | cagtggcaca | atctcggctc | actgccactt | ccaccttcca | ggttcaagca | attctcctgc |
| 65641 | ctcaacctac | ttagtagctg | ggattgtggg | tgtgcatcac | catacctggc | taatttttgt |
| 65701 | gtttttagta | gagatggtgt | ttcatcatat | tggccaggct | ggtctggacc | tcctgacctt |
| 65761 | aggtgatctg | cttgccttgg | cctcccaaag | tgctgggata | acaggtgtga | gccactgggc |
| 65821 | ccggcatcat | ttaaccttaa | ttatctccct | aaaggccctg | tctccaaata | aagtcgcatc |
| 65881 | atgggttaga | gcatcaacat | gtgaatttga | ggggtacaca | attcaatcca | taacaaaact |
| 65941 | ctttaatctg | tgtatgtttc | cacttcttgg | ttttttccat | attcacccca | tggccacata |
| 66001 | ttaatgcatg | tgtcttagtc | agcttgtgtt | gatacaataa | attccatagg | cagggtagct |
| 66061 | taaacaacaa | atacttattt | ttcacagttc | tggaggctgg | aactgtgaga | tcagggtggc |
| 66121 | agcatggttg | gggttcttgt | tgaagaccct | cttcctggtt | tgcagactgc | catcttctca |
| 66181 | ttgtgtactt | tacccggtag | agggagaaga | gagagaaagc | aagctctttc | ttatctcttc |
| 66241 | ttataaggtc | actaatccca | tcatgcagcc | tgcaccctgt | gacctgatta | cctcccaaat |
| 66301 | gccccatctc | caagtaccat | catatagata | agtacatata | gatgagactg | acattattat |
| 66361 | tgagagtaaa | ttacatatta | aatacaccca | gcttttagcc | gttaaataat | gttgtgtttg |
| 66421 | tttgatcatt | tgtagatgaa | tcccattctg | ttttccacct | ttattacttt | tcttgtgtcg |
| 66481 | tcgcaagact | ggaagggcag | acagtccaaa | tcttcatatc | gaccccatgg | aaacttgctc |
| 66541 | aatgtatgaa | tagaaagggg | accatcagtc | agactgtcag | tctccaagct | tcctctctat |
| 66601 | cttgtggact | cagatgtcaa | ataatgtaac | tttcatatta | ggcaaatgca | aatatactag |
| 66661 | gttgtatgta | tctgtatgta | ttttgatgac | tgacacactt | tggtatcggt | cagcacacag |
| 66721 | aaaggaaaat | gtacaagatt | gtactcctgg | aaagaaacaa | cttttagggt | tttcaaagat |
| 66781 | tcccatacaa | aaaactttaa | agacaaaatg | caacacttaa | ttgaaaattc | agatttgtga |
| 66841 | aaaagaaaca | aagatataga | tatattcgta | ttatattttt | attatatatt | ctagatgata |
| 66901 | tattaatatt | tgatatggtt | tggatctgtg | tccctaccaa | atctcatgtt | gaattgtaat |
| 66961 | ccccaatttt | ggaggtgggg | cctggtgggg | ggtgattgga | tcatggtgct | ggttaattat |
| 67021 | gaatgggtca | gcaccatccc | ctcagtgctc | ttctcatgat | aatgagtgag | ttttcatgtg |
| 67081 | acctggttgt | ttaaatgtgt | gtagcacctc | ccttttctct | aggcttcctg | ctccagccac |
| 67141 | gtaagacatg | cctgcttccc | cttcaccatc | cgccatgatt | gtaagcttcc | tgaggcctcc |
| 67201 | ccaaaagcag | aagctgctgt | gcttcttgca | cagtctgcag | aaccatgagc | taattaaacc |
| 67261 | tcttttcttt | ataaattacc | ccatctcggg | tatttcttta | cagtgatgtg | agaatggact |
| 67321 | aatacaatat | tataaacagc | atctgtttga | atgttttcat | attctttttc | atatcaaata |
| 67381 | ttgagtatac | tattttaaaa | tattttcact | tttcacaaaa | gctaaattag | gctgattaaa |
| 67441 | tttaggcagg | gctttttgat | ttttacttat | atgtgtcttt | cttgtttgaa | ttattttaat |
| 67501 | aagaagatac | atggctttca | gaaagaaaga | gagaagagag | aagaaagaag | agaagaaaag |
| 67561 | aaactggcag | tgccggtgta | tctgcatatt | ttctattttg | tcttgacaac | gtatgcagat |
| 67621 | tatagaactt | cttaaaaggt | catttgataa | agtgaaacaa | atgacttcga | aaaaataatc |
| 67681 | ctaagggaaa | attgtttgga | acaattaaat | tgtaataaaa | ataatttaaa | ccactttgca |
| 67741 | attagtcttc | ttatgaacat | ctacaactcc | aaaatgattt | tgacaaaaat | gaatcttggc |
| 67801 | aaccaattaa | tgctcagtct | catctaattt | atttggtttt | cttgagagca | gagaaagctg |
| 67861 | aagaagattt | ctagaaatga | taacaattta | agacattcac | acacaattgt | attttttgaa |
| 67921 | aagcgtctta | tgtgaatttt | tattacaaat | aaaaatatta | caccttttgt | tgtcatctga |
| 67981 | atacactgca | ataactttga | agttgtctga | attgttatat | tcaaagcaaa | tctgcatggt |
| 68041 | gaagcttatt | attttcattt | attaaaaaag | aatataacaa | actattttca | gtaactgtta |
| 68101 | ctaactttat | ttagatatca | ccatctgcat | tttaggatta | tctgtacaat | aacaattgca |
| 68161 | ttgttacatt | aaatgcaaga | acccctcttc | tgcttatcat | ttttaagagt | aaagcaatag |
| 68221 | agatgaagtt | gcaacagtat | tggctattag | tgatatttgc | caagccaagt | ttctctctct |
| 68281 | ctctttctct | gtctctctgt | atgtagtgac | tgtaaagcta | accatcagat | ttcaagtgta |
| 68341 | gattatggag | tgtcttatta | tgtggaaata | ggcttctgtg | agctcataca | agtatgtgtg |
| 68401 | atggttaggt | tcactatgaa | ataccttaca | gataaaatct | cttttataat | ctttactata |
| 68461 | cattcattta | tgattcatca | aaaacaacac | aaagaattta | aaactaaatg | tatgcccttg |
| 68521 | ttatttgttt | catggaactc | atttgaccaa | gatgactaga | tcgattaaga | aaggatagac |
| 68581 | acacagaact | taattgctaa | gatcaaagta | taattttagc | aaatcaacaa | ttgccactta |
| 68641 | tttcttcaat | ggtacctatg | atcaagaggt | taataataga | ggtttatgga | taggactgtt |
| 68701 | tccctgtaga | aggcagaata | ccaaataccc | caaattgcac | tgcccgtgtc | tttcaagtgc |
| 68761 | ctatgattac | aaactaaaat | aaaaacatat | acaatgcaaa | caaacgaaaa | gccctatggc |
| 68821 | tacatttgga | caatacagaa | aacaatggaa | agatttgtat | ctcccttttt | cctcccctgt |
| 68881 | ctcctcacct | cagttttttt | ttctttttag | tagagagagg | aaagaaaaga | aaagtggcag |
| 68941 | aatccaaaaa | gagggaagga | gcaagaagag | gaaagagaaa | taggaggaat | tgggcaaggt |
| 69001 | gaaaatagac | aaaagggtta | ttttagttgg | catttcactt | gttcttccaa | taaaaggcat |
| 69061 | atcccatgtt | attttgctca | ttgcctttgt | ctgtctgctc | tctctgctca | cccctgtggg |
| 69121 | cccatcaggc | caccactcat | gtctgcctgc | tgggggcttg | tgtagaatag | agccccggca |
| 69181 | gagaagggct | tgacaatctt | ttacataaag | cctgagtaaa | gatccttcag | atgtgtgtga |
| 69241 | tcttcaggaa | gccattagtt | tctttgagcc | tctgttttct | ctattctaaa | ataagaaaac |
| 69301 | tgctgatgtt | acaaagtgct | ttggaggaac | aacaaaaaca | acagcacaca | tatccacacg |
| 69361 | tgcacacata | catacccgga | actcacctcc | tgctccttcc | tttaagccta | tgcatgcatg |
| 69421 | gctttatcta | atctataaaa | tgataataat | aactttgaaa | accaaaatta | tatatgtttt |
| 69481 | ttattatgca | agtgtaaaca | ggcagtctgt | agaaaaaata | catttaaaca | atataccact |
| 69541 | ctcaaaataa | acactaatgg | ttttggtata | ttcttagagt | ttttcttcaa | tgaatataca |
| 69601 | tacatatatt | tttatcccaa | aacacagttg | tatatgtcat | tctgcaactt | gctttttaaa |
| 69661 | tgtgacagtg | caattcagac | atctttttat | atcaatacat | atagattaat | cttgctattt |
| 69721 | ttaatagttg | catacttata | tgcctataat | ttattttaac | tcatcctctt | tgtggacatt |
| 69781 | tacataattt | tccttttttt | ggctaattca | agtaatactc | agtgaatgtc | tttgcatata |
| 69841 | gattgtgtta | atttttccgt | aggataagtt | cttaaagtag | aatgtttgtg | tgaaaggtat |
| 69901 | gctccatttg | aaattttgat | ggataacacc | atattgccct | cctaaaaaga | ttacatctta |
| 69961 | tttgcaatat | ttttcatttc | aattttccaa | gctagaaact | tctgggtcat | caccaagcct |
| 70021 | ctccctttca | ttttctagct | tccttccaaa | ttgtcaccat | atcctgtgat | catactgcag |
| 70081 | aaaggatctc | ctcatttcat | ccccgcatct | tcatccttcc | agccagtttc | tctttgctca |
| 70141 | tgtcttatag | aagaaacttc | caaaagttat | ccacaatcca | ttgtccttgt | catcctgggc |
| 70201 | acttgagaga | ggacatggtt | ttctaaggct | ccctggtatc | tacctggcca | tgccaatgga |
| 70261 | aagataaata | aaatggcaca | tgccccttgc | aggcttggcc | cctaggaacc | tgccattcac |
| 70321 | attcttcctg | ccatttcatc | ttctactggc | tgaatgttgc | cagttacaag | gccctgtggg |
| 70381 | aggtggaact | gcacatggca | gagcctgggt | ctctgagtga | acatgccaga | ggacagctac |
| 70441 | cccacagtcc | tgagtgactg | cccagagcaa | tgcctcttgt | gttgagctat | gacatggttg |
| 70501 | ggtctatttg | ttacaacagt | tagacgtcag | tagcagaagc | agagaggccc | catcattttt |
| 70561 | tactcacagt | atgagagcag | ccttcttgtt | ggcctctcta | actcctgctc | cttccccttt |
| 70621 | cagaagctct | gctgccagaa | taatcccact | ttgaaccaca | gtgacctgta | actccttcct |
| 70681 | ttaaagtctt | caggaactca | ttccctatag | attcagatcc | agactctttg | gcctggaatg |
| 70741 | gaagatcctt | tataacatgt | ccacagcctt | ccatggggta | ttgggggcag | agtcctcatc |
| 70801 | tatcttgtcc | tctgtgctag | acaatccaac | atacagtagt | gaaaccatcc | cccttccttc |
| 70861 | tcccctgatg | aaaactataa | agtgtataaa | ctgtagagtt | aatcagggga | gaaggaaggg |
| 70921 | ggagaaataa | aaataagcca | agcttgaagc | acactctgca | ttagtcatga | ggtcagcctg |
| 70981 | cttcctcata | gtggtttgct | gcctgttgcc | tcagaatcat | gtagaccctg | ttacaagata |
| 71041 | aaagttcccc | tttactgatc | tacagataac | ttgaacatta | caaagaattg | ttttcccttt |
| 71101 | gagatattct | ctcagttcct | gcataccaat | aaaactacta | gtgactcagc | tggtctgaag |
| 71161 | aaccccacag | gaggtgattc | accaaagaat | acagtttcca | catcctgatg | atttcatccc |
| 71221 | ccttaccctg | aaaaatgaac | aaccccaatt | ttccagcccc | tcaccctcca | tgtctccctt |
| 71281 | aaaaactcca | gcccagaatt | cctaggggag | atggttttga | aggggctcct | cccatctcct |
| 71341 | cactctgctg | ccttgtgatc | attgaactct | ttctctgctg | caagtcctgc | tgtctcattg |
| 71401 | tattggtctc | ttactgcaca | gtgggcatat | gaacttgctg | cttctctaac | agtagcattc |
| 71461 | aatacatact | tgcagaataa | atcaattcat | attgtttgct | ttagagacaa | atataatgca |
| 71521 | acatcaagga | ggcagtttta | taaaaacaat | ttgctttgta | gggttattaa | tttacgctta |
| 71581 | gcaattgcta | aaaatactga | tttcatggct | cacctcttga | gatagggtaa | aacagcatgt |
| 71641 | ttttgatatt | gctgatttag | gtccataaca | ttctaagcct | aaaagggcct | tagagctcat |
| 71701 | ctagtacaat | tttctcattt | tgcagataaa | aacactgcat | tccagacagc | tgaagagact |
| 71761 | ccaggctatg | aaaccagtgg | gatacagtcc | caagcaggcc | agcacccagt | tcataggtct |
| 71821 | gtctgccttc | tgtgacacca | ggctgtccct | aaggctaatg | gccctagaca | acttgatctc |
| 71881 | tgcagtctaa | tgtctgctta | tattcaactg | gctttgtggc | tccatttaag | gttttatttt |
| 71941 | ttggtgcctc | atttgtcaat | ttggtttccc | agtcttggtc | tgactgggaa | gaagcagagc |
| 72001 | cagtgtacca | tgcaggggac | gggatgttag | atctggcctc | ttgctctgca | ttcctctgtc |
| 72061 | acccatagaa | tttattgagc | ttcctcatgt | acaggtcctt | cttcattcat | cagggccaga |
| 72121 | ggctgctggt | gagcagggag | acattttttc | taacatggtg | ggaggaatgg | cagttttata |
| 72181 | actataatta | taccctccag | aaaataactg | ttacttaaaa | ggtgtatcgt | tctttgtaca |
| 72241 | tggatctgaa | ggctcagcga | tgggtacatt | taaaaatcaa | taaagtagtc | agccaaacat |
| 72301 | tcagctcatg | catcacaagc | tcacagggat | tcagatttaa | cagaaagatg | gggtaggcac |
| 72361 | tgcagtggtt | tgggccatgg | gcacctactg | tgtaaaggaa | cagaaacatt | gtaagacatt |
| 72421 | tcaaattgcc | ctgcacttgt | tcaggttagg | ttcatcttgt | cagatacctt | tcctattctc |
| 72481 | tctggtactt | ctccatcact | taggatgaat | ggtgaagagt | tttctggcaa | agtgcttgtt |
| 72541 | gcactttggt | gttaaccagc | tattgacagc | cacacacttt | gaagtgactt | ctcctacagg |
| 72601 | agtcactgat | ggacagcaga | gtgtagtgca | aatatcagga | ctgaggttgg | gaggtttaaa |
| 72661 | ttacaggttt | caaatgattt | tgggaggaaa | acttccattt | tcaaacaggg | tgaattatga |
| 72721 | gaaacaacct | gaaatgccac | ctttatgcct | cttcttataa | cctaaaaata | cagaatggtc |
| 72781 | ctgagaaagt | ttgctttggc | taagtagcat | atttgtctgt | aaactatgaa | attgctcatt |
| 72841 | cattcatcca | tacagtcatt | aagttgatac | atatttatta | agagtatcct | caacaccatt |
| 72901 | gaatatatac | tcaatgagtt | gttctatgtg | gtggaattaa | caataatgac | aaaccaagcc |
| 72961 | cctttactca | tggagtttac | atttcaatgg | aataagaata | ataatataac | aataagaaaa |
| 73021 | cataataagc | catgagagct | gcattggtta | tattttagtt | tgttgaaaaa | acccctcaaa |
| 73081 | ctaagtatct | taaagcaaga | gttgttcatg | atttcccact | attttgtggg | caagagggtg |
| 73141 | ggtcatttgc | tgccctcacc | tggactcaca | cttgtggctc | ctttgaaccg | gaaggtcagc |
| 73201 | tggtggctgg | gcttggctgg | gcttggctgg | gcgcactggg | tggccaggcc | cttctgacca |
| 73261 | catcatgttt | tgtctctggg | tggtcttagg | gttccatggg | aacaagtacc | aaagagcaag |
| 73321 | tgcttattga | gtttctgtgt | gtgtcatgtt | tgctaatgcc | ccacaggcca | gagaaagtca |
| 73381 | caaagctagg | ggcatggata | tgagaatgca | tgattcatta | ccgaccagta | gagaaacaat |
| 73441 | ctaacacatt | ggtaataaga | ggcaggaagc | aaactaaaga | atgagggaat | caacggagag |
| 73501 | tgatggggca | gttattttag | ataaggagat | caagatgact | ctctttgagg | agagaacatt |
| 73561 | gaaacagaga | cctgtgaagt | agggtagggg | gtcaagtgga | ggtaccaaga | gagtgttcca |
| 73621 | gccagagggt | tagcatgtgc | aaatgccctg | agtgggaaca | acagggagcc | agtgtggctg |
| 73681 | gagggcagta | agccaaggga | gaatgttatc | atagtggtgg | aaccttgtag | gctcttgcag |
| 73741 | accacggtaa | ggacttcaca | aggtgttcta | agatggaagg | aaacctgcac | atgctcgttg |
| 73801 | tagaaaaggg | aagaatttga | tttatgtttt | aaaaggatca | ctctggcttc | tgtgtagaga |
| 73861 | ataaacctta | taagagcaaa | agtggaagca | ggaaaagaag | ttaggagaaa | agagagagtg |
| 73921 | aggtggcttg | gaatgcaatt | ataacagcgc | agatggtgag | aaagagacaa | actcagcata |
| 73981 | tgttttgaaa | tcagggctca | cggtacttaa | aatggattga | atgtgggtta | tgagagaaag |
| 74041 | gcatgaagga | tgccttctag | gttattgtct | gaccaccttg | gtgagtggtg | tttctattac |
| 74101 | gagagagatc | gcattggctg | ggtcagggta | gaatgcaaca | tagaagatag | cggacatatt |
| 74161 | tagaaaatga | aatcaatgac | attgcctcac | aaaattctaa | agctgaaaat | ccagcaactg |
| 74221 | acataatccc | atcattttat | agacaaagaa | attgggatct | gggaaaatga | aggaatttct |
| 74281 | ttttttaaaa | aacactttct | gtaatcccag | cactttggga | ggctgaggtg | ggcagatcat |
| 74341 | gaggtcagga | gatcgagacc | atcctggcta | acacggtgaa | accgcgtctc | tactaaaatt |
| 74401 | acaaaaaatt | agccaggcac | agtggtgggc | gcctgtagtc | tcagccactc | gggaggctga |
| 74461 | ggcaggagaa | tggcatgaac | ctgggaggcg | gagcttgcag | tgagctgaga | tagcactatt |
| 74521 | gcactccagt | ctgggcaaca | gagtgggact | ccatctcaaa | aaaaaaaaaa | aaagacattt |
| 74581 | attaggccat | ttttgcattg | ctttaaagga | atactgggac | tggataattt | ataagaaaac |
| 74641 | aggcttaatt | gacttatggt | tctgcagact | gtacaggaag | catggtgcca | gcatctgctt |
| 74701 | ctggggaggc | ctcaagaaac | ttttattcat | ggcagaaggc | gaagtggtgg | caggcagttc |
| 74761 | acatggtgaa | agcaggggcg | gggccgggag | gggggaggta | ctgcacttta | ttagtttcat |
| 74821 | cctacaggtg | aaattagttt | gctgataaat | gtcaaatgct | ttaagatttc | ggagaaagtt |
| 74881 | atttagatac | tctgactcta | atttttttgt | ggagggagga | tcacattcga | taaacactca |
| 74941 | agagaataag | atagaggaat | gaggcaccgg | cagaagggtt | ttgtattagt | ctgttctcac |
| 75001 | cctgttttga | ataaatacct | gagactgggt | agtttataaa | gaaaagagtt | taatttactc |
| 75061 | acagttccac | atagctggag | agcctcaggg | aacttacaat | tacggtgaag | gcaccccttc |
| 75121 | acagggcggg | aatagagaga | aaatgagtgc | cagcaggaga | aatgccagat | gcttataaaa |
| 75181 | ccatcagatc | ttgtgagact | tgctcattat | catgaaaaca | gcatggggga | aactgccctc |
| 75241 | atgattcagt | tacctccacc | tggtcccacc | catgacattt | gggaattatg | ggagctacaa |
| 75301 | ttcaagacga | catttgggaa | ttatgggaac | tacaattcaa | gatgagattt | gggtgggaac |
| 75361 | acagtcaagc | cattttaggt | ttggtggaat | tgcttctttg | aaaatacagt | tgcagggacc |
| 75421 | tgtcttagtc | cttttgtgtt | gttataaagg | aatacctaag | gctgcataat | ttataaagaa |
| 75481 | tagaggttta | ttttgctaac | cgttctacaa | gctgtgtaag | aagcatggag | ccagcatgtg |
| 75541 | catctggtaa | gggtttcagg | ctgcttccac | tcatggcgga | aggggaaggg | gaaccagcat |
| 75601 | gtgaactggt | tgttaaaaag | agcagggcac | ctccctcccc | tctcttgttt | cctctgttgc |
| 75661 | ccatatgatc | tgcacatgct | ggcacccctt | agccttggcc | atgaatagat | ctaccccagg |
| 75721 | gtccaaacac | ctctcaccag | gccctacctc | caacactggg | gatcatattt | caacttgaga |
| 75781 | cttggctgag | tcatacaaac | cagatccaaa | tcatagaagg | accatagcta | gtttggatag |
| 75841 | attttaccat | caagaggtct | acccagacaa | atgaaagatc | tgtgagcatt | tgttgaatga |
| 75901 | atcttcaaca | aagtttaggc | attttgaata | tttaggtttc | cttgttgaag | cttgaaaata |
| 75961 | tgccttttgc | atttgacaaa | aattaagtta | cattctctga | agtctgttga | agtaaatttt |
| 76021 | tgtagtcctg | tcattcattt | cccactttat | tgagtacact | gctggtattt | ttggttgttg |
| 76081 | tttttaagct | ttctttattt | tccaatccaa | attgatgtgt | atcaggctgg | tatttttaat |
| 76141 | gttaatgggc | actcaaaatt | tagtgttcag | agaggtagag | aaggcaaaag | aaataggctg |
| 76201 | aaatttatct | taaatcaaag | agccaggcta | aaaggagtct | atgtcaaggg | ggcaggaggt |
| 76261 | atggttacta | cgaatttaaa | taggattccc | cttgtatgaa | attagcagtt | cttcattact |
| 76321 | cagcacgtcc | ttccccacct | atttccttca | tcatacttat | catccagaga | ttgctgaaga |
| 76381 | cagaaacata | ttatgatgac | ctttattagg | tgtttactat | attccaggtt | tgtagctaag |
| 76441 | tgctttataa | acattacctc | attattgttt | tgtccctaga | gtcaacgctt | atctgactct |
| 76501 | aatcttccta | gaagataggg | ccctggtttt | ttgatatatt | cactatacga | ccttcagcaa |
| 76561 | gtcattaagt | tccttatgcc | tcaggtttat | aatttatgaa | atgcaggatg | gatttttgtg |
| 76621 | tatactaccc | ttcaaatttt | atttctcttt | aaaagttatg | acttaaaaat | aattccatgt |
| 76681 | ttggctttat | ctaaggagat | gagggcatca | atggaaaagg | actttgaaaa | atgaaaaaat |
| 76741 | gaacaggtcc | ttctagaagt | tcgagttttt | gagaaaaaaa | ttgcgggaat | gagaagtctt |
| 76801 | cctttcccta | actcttttta | ctgctgattt | tatttattta | tttatttttg | agatggagtt |
| 76861 | tcactgtttt | gcccagccag | gaatgaagtg | gcacaatctc | ggcttactgg | aacctctgct |
| 76921 | cccccaggtt | caagagattc | tcttgcctca | gactcctgag | tagctgggat | tatagacacc |
| 76981 | tgccaccatg | cctggctaac | ttttgtattt | ctagtagaga | tggagttttg | ccatgttggc |
| 77041 | caggctggtc | tcaaactcct | gacctcaggt | gatccacccg | cctcagcctc | ccaaagtgct |
| 77101 | ggaattacag | gcatgagcca | ccatacctgg | cctcgtttta | ctgctttgaa | tggcaagatt |
| 77161 | cccttatgtg | tttcctagtt | caaattattt | tgttgtagtt | gtcaaaatta | ctttgtcctg |
| 77221 | tttttggagg | caagcatata | gtcttattta | ccaattagtt | tcagaaatgt | gagtagcata |
| 77281 | gcttctcaca | taggcagact | ttgtcaagga | tgatgtggga | ggaatattga | aggataagta |
| 77341 | gtccttagtc | tctgtgttag | cccattttct | gtagcttata | acagaatacc | tgaaagtcgg |
| 77401 | taatttacaa | gaaaaggaag | gtatttctta | cagttatgga | ggccaagagt | ccagggttga |
| 77461 | ggagctgcat | ctggtgagag | ccttcttcat | ggtagggtcc | acgtcagctc | aaggtatagc |
| 77521 | atggcaaggg | ggctaagcgt | gttgatgggc | tagctcaggt | ctctcttcct | cttcctataa |
| 77581 | agccaccagt | tcccctccca | tgataaccgt | gaatccacga | ctggattaat | ccattcaaga |
| 77641 | gggaagagcc | ctcatgatcc | aatcatctct | tgaaggcccc | acctctcaat | actgacacat |
| 77701 | tggggattaa | gtttcaacat | cagttttgga | ggagacaaac | atccaaaccc | tagtagcttc |
| 77761 | cagtacagaa | ttgccaatac | cttaggtact | tccaagtaga | gaattccccg | gaaaatctat |
| 77821 | tccttggttt | tggagattac | tctccaagtt | ctcgccaact | tgagtttccg | tggttctgtg |
| 77881 | agattctttt | ttcaagtaat | tcaagggttt | tcaggtagat | actgtcatag | attctgtttc |
| 77941 | aaatctgcca | attctactga | acacaaaaaa | aggttttttt | taatccagat | ttttttcttt |
| 78001 | tttttttttt | gagacagagt | cttgctctgt | tgctcaggct | ggagtgtagt | ggcacgatct |
| 78061 | cagctcactg | caaccactgt | ctcccgtgtt | caagcaattc | tgcctcagcc | tcccaagtag |
| 78121 | ctgggattac | aggcacacac | caccatgcct | ggctaatttt | tgtatcttaa | tggaaacgag |
| 78181 | gtttcaccat | gttggccggg | atggtcttga | tttcctgacc | tcatgatctg | cccgccttgg |
| 78241 | cctcccaaag | tgctgggatt | acaggctgat | ccagattttt | aaaaggttta | cttgagtctt |
| 78301 | agtttcatat | tatgaacaga | caatctctaa | gactgaagtt | taaaagttgc | actgaagtac |
| 78361 | agacttcaag | tccaagttta | caatttcagt | ttctcactta | cctcatttct | gatgtgcttg |
| 78421 | ttattttatc | tatattctgc | agagtgaaaa | agtgcagaat | gagagtcatt | aaggtctaaa |
| 78481 | ttggttgttg | ctaagggtct | aaattactgt | tgatgatgat | gtagcccttc | tggtggccta |
| 78541 | gctttgctgc | ttttggtgct | gagaggtagg | gcgaaggtca | tgggacttga | agtgggaggc |
| 78601 | aggaggaggt | gtgcggggtg | tgcagggtgt | gcatcctgca | gtttctgctt | ctgacattca |
| 78661 | atcttccacc | tttgaaactc | ttttttgggc | caggggtcac | cacttactat | aggtagaagg |
| 78721 | cagtcccaat | tcctccctaa | caggctgggc | gcaatgactc | acagcacttt | gggaggctga |
| 78781 | ggcgggcaga | tcacttgagg | tcaggagttc | aagaccagcc | tgtcctacat | ggtgaaaccc |
| 78841 | tatgtctact | aaaaatacaa | aaagtagctg | ggcgtggtgg | ttcacacctg | tagtcgcagc |
| 78901 | tgcttggaag | gctgagccag | gagaatcact | tgaacctgag | aggcaaacac | tgcagtgagc |
| 78961 | taagatggta | ccactgcact | ccagcctggg | taatagagca | agactccatc | tcaaaaaata |
| 79021 | aataaataaa | taaataaata | acaaaaagga | aaaagaaaca | tttaagtatc | cagtgtagac |
| 79081 | aagtatccag | ctttgtgtag | atagagaggc | cattctcaca | gatttctttg | ggtctgcatt |
| 79141 | atcttcccaa | accacaaccg | gtttttggcc | cagaaattca | aatttgctcc | ctgaagtctg |
| 79201 | tttttcttct | atgttgagct | tgtcactgaa | aagtcagttg | tcaatcttaa | cataatcccc |
| 79261 | tttctacaaa | ttccaactat | ttggttcttt | tgtctctcta | attattttat | tcaaaactat |
| 79321 | ctattcttgt | tttgggggag | aggagatttc | ccttcttatt | aaaaaccact | tagttatcag |
| 79381 | tgattataag | atcagcaatt | ccttgaggac | aaggattatg | ggttattcat | cattgtatgt |
| 79441 | tcactcatgc | ttaaacactg | gacaattttt | tttttttata | agcaagctac | agacaaccaa |
| 79501 | aatggggcaa | gggaattcaa | catgtaatcc | atgtttacca | aaacgagatt | tcctgagatt |
| 79561 | tccagcatag | caaatgtatg | tttctgtact | tttcttcctt | ttcttcaagt | aattgcaacc |
| 79621 | aactttctag | gctatgtacc | tgttaatgtc | agaaattgtt | ctctgtctct | ctctctgtct |
| 79681 | tccatgttgc | caagtgttgt | ggacagcaat | ataaattcct | taaatgaatg | aatgcgattt |
| 79741 | ttcaagccca | gttagtttat | ttttcagaag | aaccaagttc | tagtgaatta | aacttttatg |
| 79801 | atctagcaaa | tccgtcacac | aaaccagttg | ggaaaacacc | tataatttat | gcttcacaga |
| 79861 | ccttctgtct | acattctaaa | attttatttg | cagtgcctac | actgatttga | ataaattgaa |
| 79921 | aaccaaggtt | ttttttcttt | tcttttttca | aataaaacaa | acgagggttg | aacagttttg |
| 79981 | tatatttcga | attgtaataa | ttttatttct | ttcttcattc | cctgtttctc | ctatgaaatt |
| 80041 | aaaatgcaag | atttaaaatg | tgttgaattg | ttaagtatat | tttgtattta | ttttagacta |
| 80101 | aatcagagta | attaatagaa | attctaaatg | gaaatcacct | aatgatattg | ggttcaaatt |
| 80161 | ttgttgaagt | taatttaatt | tttattaata | atttatcaaa | tatagctggc | ttttgtgaat |
| 80221 | tatattaaaa | cgtatctttc | tgtgaagtgt | tgaggccaag | aatgaaacct | tagatggaag |
| 80281 | acccagattt | actggaagta | ggccagaaat | tgtaatcaaa | ggttttggac | tttctgctct |
| 80341 | gggtttaaat | tctagtccca | gcagttttta | gaagcaaggt | taatgaatct | ctgggcacct |
| 80401 | atgagaaggc | gtgttttgaa | ggccttcccc | aaagccagcc | ctctctaagt | ctctggcgtt |
| 80461 | cttttctttg | aatggatgta | cttaggagtc | cacagggagc | tatggacaca | gaggagagaa |
| 80521 | gaaaaataaa | attaaaaaaa | aaaaagctaa | tttcttggtc | atgattgttt | aaaaagaact |
| 80581 | tgcaaaagaa | aagaaacaga | agggtcaaaa | agacgaatac | aggttgagta | tcccttatcc |
| 80641 | aaaatcttgg | gaccagaagt | gtttcagtct | tggattttta | gggattttgg | aatatttgga |
| 80701 | tatatgtagc | accccaaatt | tgaaaatttg | aaataaaaaa | tgctccaata | agcatttcct |
| 80761 | ttaagtagca | tgtctatgct | caaaaagttt | tgaatttaga | agcattgtgg | atttcagagt |
| 80821 | tttggatttg | ggatgctcaa | cctggaaaag | gtaactcctg | ctcacttcca | cccctcatgg |
| 80881 | agaaatttct | taaaacagaa | caagctctca | tgctccattt | ccatcacagt | acacattgtg |
| 80941 | aggacatgaa | acataatgtt | ttcagtgttt | ggttatatga | gaatcaaata | gcttcctggg |
| 81001 | gaagaaaata | ctgaatttta | cacaacccat | atgccactaa | tgggatcaga | tggcaatata |
| 81061 | gtaacacgca | gcccagttct | tcatgcttca | tccaaaaacc | atgccacgga | tttggggttt |
| 81121 | ggacactgaa | tttgtcactg | tctgcatgat | cttagataag | ttactttatc | tcagtagtgt |
| 81181 | aggtggcatc | atcggcccac | catcacgact | gatggcccct | gagctgacaa | taaaaaacaa |
| 81241 | gtgtacatct | ttaataccag | aaagtagtca | gtaaatatgt | ggacgtgtgt | tgtgagtggg |
| 81301 | tcttatctgt | accatgagag | gtgttagtct | gagatctcta | aaaagggagt | gcctagaaac |
| 81361 | caagcatgga | aatgtgtcaa | accgtcacca | agagtgaaag | atgatcagga | accagagaaa |
| 81421 | tagccccaga | atagagctta | cccacaccag | gagcagtaac | ccctggctac | cagtaaatcc |
| 81481 | cactgcttgg | tctttgctcc | tctctgacat | attttattgc | agcccaagct | catggacagg |
| 81541 | ctctcatccc | atcaccctca | acatcacctg | tgtttcaggg | ttgttctgag | aatcaaacag |
| 81601 | gatttaccct | aagctcttca | gggatgtcag | tcaaggttag | catgatgcag | ggcctataag |
| 81661 | tttactttaa | gagtatttct | caagagtcat | cttctatcaa | aggcagtctt | ttccaagtca |
| 81721 | gcttgaggcc | acacagtatt | ttgaaaacat | cgtggacttt | ggagtaaaaa | caaatgtctg |
| 81781 | caatttgttg | tgttctgtta | ctacggattg | gcctgatttg | agatacttat | ttaacttctt |
| 81841 | tgagcctctg | tttcctcatc | tttcatgaaa | ctaagatact | gtctctaaat | ggtgtcacaa |
| 81901 | agtagtaata | taataattaa | cagctgaata | tgaataatgc | cattatgcct | ttctggtaag |
| 81961 | actcactgta | ggtaaacact | ttgacctatg | ttattttatt | tgatctttat | agaaacatgg |
| 82021 | gaggatagtg | atgtgcctac | ttttttgaat | ggcctgctag | gtaatttgtg | ccaccctgaa |
| 82081 | agccagccta | atggacaggt | agtgcaaaag | aagcaagagg | gtgttcagtt | tcctgcatga |
| 82141 | acagagtcag | caactgacat | ctgagagcct | ttggggctat | ttcatcctgg | ggtttcaagc |
| 82201 | tcagcttcaa | agggactgga | gaatgaggca | cagatgagaa | gatggggatc | ctatctcctc |
| 82261 | cccaacttca | attgcagcag | ctacatttct | atgtgttatt | gattgaattt | ccatgtaaga |
| 82321 | ttccaatgga | agaaagttgt | agtcaagcac | tttcatttcg | caaatggcaa | aactgaagcc |
| 82381 | gatattgtgg | ttgtgactta | tcccaaagta | atatacacat | aaacctctat | ggatgaggaa |
| 82441 | aaagacagag | ggaaactaaa | aattcaaaag | aacaaatttg | actcacagat | ttgctgactc |
| 82501 | atagttatga | tagttatttc | ctctacccac | gctttcttga | ctgattgaat | aagttgcagt |
| 82561 | ttttgtcctc | aattaactca | tcggaacatg | acattatttt | aaactattga | accaccttct |
| 82621 | tcattaaata | gtattttttg | agagtttact | atatatgtca | ctgtccaggt | acaatgaaaa |
| 82681 | ataccataaa | gataaagcat | tctacactcc | atatggatag | aattcagatt | gcgtagatga |
| 82741 | ctcagtctta | tgagaaggtg | gtatataatg | acaatgacaa | ataagtgtga | ccatcttttt |
| 82801 | tttttttttt | ttttgaaagg | atctcactct | gttacctagg | ctggagtgca | gtggtgcgat |
| 82861 | ctccactcac | tgcagccttg | acctcctggg | ctcaagtgat | cctcctacct | caatctcctg |
| 82921 | agtaactgct | accacagtta | tgcaccacca | cgcccggctt | tttttttttt | taattgttag |
| 82981 | cagagacagg | atcttgctgt | gttgctcagg | ctggtctcca | actcctgtcc | tcaaggaatc |
| 83041 | ctccatgcct | tatgctagga | ttacaggcat | aagccaccgt | gcccagccag | tgtgaccatc |
| 83101 | ttgaaaggag | aaattacaca | aaactcaggg | tgctctaggg | aagtttattc | agaagttgag |
| 83161 | gttttagctg | gtccctgaag | aatgaataat | attaaaatag | gagacaagaa | gacacgagat |
| 83221 | tccaaatgta | gaatatcttc | atagacttgg | aaatctcaag | gctcctactg | agaagatgaa |
| 83281 | aacaaaattg | gtatatctag | agctaaaggt | ttatgaaagg | aagttggtaa | ggcctggaat |
| 83341 | cactaagcat | tgaacagtgg | atcatcaggt | gacagctgag | gccgttcctc | atattacaga |
| 83401 | agcaggaaat | gtgttccagg | agccagctcc | atcgtctaac | cctacagtat | ggggctgtcc |
| 83461 | ttccccgtcc | tatgccatct | ctctgccccc | actggatcaa | attcacagat | aacaagattt |
| 83521 | aacacatcat | ttcttaaact | cctactttgt | caccaccacc | tctactcaat | tcccaataaa |
| 83581 | ttggaatata | cataacacag | gaagcaggaa | acaatagaaa | ggctttgagc | aaggcacagt |
| 83641 | gtctttgaaa | gatggattta | gtgagtatat | ttgaactgga | aggatctata | ggtaaatgta |
| 83701 | ggcaaggggt | ctgtgaagga | atccatttat | ttattttatt | tattattatt | tttttgagat |
| 83761 | ggagtctcac | tctgtcaccc | aggctggagt | gcagtgtcac | gatctaggct | tactgcaacc |
| 83821 | tccacctccc | aggttcaagt | gattctctgg | ccgcagcctc | ccgagtagct | gggattacag |
| 83881 | gtgcccacca | ccacacccag | ctaatttttg | tattttttag | tagagacagg | gtttcaacat |
| 83941 | gttggccagg | ctggtcttgg | actcctgacc | tcaggtgatc | cgcctggaaa | gaatccattt |
| 84001 | atatgaacat | ttcttgtgtt | cctatgtgat | gtgtgctcta | tgtattattt | ccaatttaat |
| 84061 | tttaagacaa | cccaattagg | ttgataataa | tatctcaatt | ttataaacac | gcaaactaag |
| 84121 | gcacgatgag | atttaagtaa | ctttcccagt | ctcaattcac | tagcaagtaa | gagctggggt |
| 84181 | ataaaactaa | acctttataa | tgttaaagta | catccccttc | tttattcttt | atttttaggt |
| 84241 | caaaagcaga | tggttgagtt | aattattttc | ctcagttcaa | gacacattat | catctttgat |
| 84301 | gtgtgcaggg | cccctttgga | agttttattt | atttattttt | atccttttaa | aaaaattcct |
| 84361 | atgccctgtt | ttcattcctc | tcttttcaca | gccaaaaaat | tcagtgtgtt | tgatgtgtat |
| 84421 | ttgttatttt | atatgtgttc | ttgaaaaatg | agtattatta | ttttgtgtgc | atatattgtt |
| 84481 | aatttatgta | agtgatatta | attatgtatc | ttcctccttt | actttttttt | cacgaaggac |
| 84541 | tattttttta | aagactcata | catatttcta | tggatgcttc | taatccattg | cttctaactc |
| 84601 | caacttctgc | cctttcaatt | acatacaatt | aattccctaa | agcaaggggc | agactcggga |
| 84661 | gacatcactc | ataaatatta | attaatcaat | taacatatta | aaagcaattc | agtcaacaaa |
| 84721 | acatttgtta | agcacttact | accagacact | gtgtttaatg | ttgagaatac | tgaaatgagt |
| 84781 | aaagtttaaa | acctaaactt | aggggactgt | cagtccagta | ggagacacta | tcatggaaat |
| 84841 | aaataattac | agtttagtga | tttatatgca | gtcatgggta | cgacctcaag | aaagagggac |
| 84901 | agcacagaca | gggtgtgatt | gttttcattg | ggagcactgg | gcaaaatgag | tgaaacgtgg |
| 84961 | aataaaaaga | atgacctttt | ctgagtttca | aagaatgtgt | aggaggaacg | tcctgagtgg |
| 85021 | atgggtgggg | aaagggtgtt | ctgggaaaca | gcactttcga | caagaattgt | tgcattccct |
| 85081 | gtcttctctg | gggcccctta | aagtccatgt | ccactcacag | gctcttaaaa | cagcagccat |
| 85141 | gtttactctc | ctacttcctg | agctgatagt | cttgaggagg | gcctctggtc | cctgctacac |
| 85201 | agccatccct | gatcagtgct | ccagcagcca | tgcgcatgtg | tagtcttggt | taaacaggtg |
| 85261 | agttggtcct | gtctactgtt | tccctggaga | atttgaacca | aaagatccag | gcatgttgcc |
| 85321 | atagcagtgg | tgaccggaag | tggagctggg | agatcttgat | gcctggtggt | catctgcatg |
| 85381 | ctaatgaata | aacaaagcgt | gggcttccaa | gcaaagcgag | agaagaaagg | ctcccctggg |
| 85441 | taaggagaaa | cgccatcttg | ctttctgaca | cttttctcag | ctctactctt | gcgagctgga |
| 85501 | ttttgcttcc | cgtcttgagg | tccatgaagt | ttatctgagt | cttgataatc | aatatccctt |
| 85561 | attgtcaact | attttgtgtt | tcaattgctt | attacccagt | gatgttgaat | aaagcatgta |
| 85621 | ttccaaggca | taaagacccg | agagactgtg | atctgttgac | agagctataa | tccccttgtt |
| 85681 | attgacaggt | atgaaatagg | tgggtggggt | tggaggaaaa | gctgaaggaa | gaaccaaaaa |
| 85741 | gacttagtga | ctgattagaa | atggcacaca | gagataaagc | aaatctgaag | ttgcaagctt |
| 85801 | ggaagcccca | gaagaaaggt | ggtaatattg | atataaagct | agggaaatga | atgaatagag |
| 85861 | aagaattcaa | tttttaaata | catgagaagt | ctttgggaaa | catgtgaaca | tcaaatggta |
| 85921 | aattagagaa | agatgagctt | agatgcctgc | actaacctta | gagaggaaaa | ggaagaggac |
| 85981 | tcagtgaagg | ggagttagga | atgagaccaa | gagtggtcaa | agatgggggt | tacagaaaaa |
| 86041 | tataaaaaag | tagctgttga | taatagtaaa | aaacacagtc | gagtccacga | aaatgaggat |
| 86101 | tatagaaaaa | aatcaatgct | caacagccct | ataaatcctt | tcatttcaga | agttttcagt |
| 86161 | gcctttaata | tgcttcccca | tatctgtcta | tgatcacatg | gatattatct | tgggtagtaa |
| 86221 | attcctggtc | tatgggaatt | tgctctgtta | aaatttgctt | tatttaatac | tcaatacaat |
| 86281 | gccgtgtgta | cttaccaaat | atgtttttat | taataagacg | aagaaggttc | tcgtgctaaa |
| 86341 | gttctcttga | ctgctaaaat | tagaaagtgt | caattttgac | ctctgtggct | gtggggagga |
| 86401 | acatctgaag | cctcgatgtt | cagatgtgac | atataatgaa | gcctcttctc | tatgcatcct |
| 86461 | tcaagtggtc | ccagctaatt | tggatgggga | attttgaggg | aatttaaatt | tggggatttc |
| 86521 | ccatgtgaag | catcactttg | caatcatgtg | cttagctaaa | aattctcctt | ttcgaggatc |
| 86581 | gctcatgcta | gtgacgcgtt | gttagtttgg | agagagaata | aaggacaagt | gttgccttcc |
| 86641 | ttcctgtgcc | gcagacttaa | ccaactaatt | aaatccactt | tgctttctgg | gcttatggtg |
| 86701 | actttgtgta | attcagactg | gaatcttgtc | acatccagaa | gatctgtcgt | tgtccaggtt |
| 86761 | accatggcag | ctaatttttt | ttttcttttt | gtagaattat | ccccagccct | tggcttcagt |
| 86821 | taaccacata | agcaggtgta | ttattagctt | aatgaaggga | ccctttctgc | aggcttcttc |
| 86881 | ccatgctgtc | aggtagctgt | ccaactcgca | tgttgttgct | gtgtttagta | tcttattctc |
| 86941 | agcatccatc | tggagctctt | ttacaaagcc | ggtaggttgc | gtgccagaga | acatcttgct |
| 87001 | gcctcggaag | aaaattcatc | tccttttttt | ttcttaaatg | tgacccatga | catcacattg |
| 87061 | tcctccccag | cccatcctga | gctctgcact | gaggggtggc | tttgttgcca | tcctaggcat |
| 87121 | gcatagctat | tgtctgtgca | atgcttcatt | ttgcaagtct | gtagctataa | tgcaacatac |
| 87181 | tctgggatca | gtttcaaagt | ctaacgatta | acacatgttt | attcattcca | aatggagcca |
| 87241 | cggctgcagt | ccctgctgca | cacatgttat | tggggatttt | ttaaattgtt | gttattattt |
| 87301 | ttcctctcta | ggttgattct | tcacatggat | cagaagcaac | cagcaatgaa | ctcctgaaat |
| 87361 | ctttgtttac | tgcctttgag | gaacctttgt | acccttgccg | tttctccatg | aggtgtgtcc |
| 87421 | ccagatcact | ttctggatgg | caggcaatgg | ccgctagaag | gtcagttttg | tgccctgctt |
| 87481 | taagtgtcac | tctgtatgca | tgctcccaca | gtgtaatgac | agccaagtag | tgacaaatgt |
| 87541 | agccttacag | actcaaggca | atagtgcaca | ccagaaaaac | acagatgggg | aaaaaagcaa |
| 87601 | tttttgattg | aatctaaaat | atggcaggta | aacatttaat | gacacaaagg | cagacattca |
| 87661 | caaagaggtt | aagaaattag | gacatatttt | tttgtaagcg | aaaaaaagtc | aaacataggg |
| 87721 | ttgtcattat | cagctactgc | agctggtgct | cacacatcct | tgggaggcgt | cccagagtgg |
| 87781 | catggtcagg | ctcctgggca | gagatggtaa | actactgacc | cattaccctc | agaatgacag |
| 87841 | tgatgtcagc | tctactcagt | gacccaggga | gacagcagca | ttaaccagta | tgcaaaaaat |
| 87901 | atagatgaaa | tttggagcaa | attgatttac | tgagggatta | cttttggtta | aatgattttc |
| 87961 | cctcctttct | gagccctatc | catcccaagc | cgaattctct | gagctaaatt | gaagtttctg |
| 88021 | caaatgcatc | ctagcagtgg | aggttaaaga | gtcactgtga | aatttatttc | atccctgaat |
| 88081 | ttacagcttt | gttaggcaaa | actatataaa | aaccataaaa | agaaagaggc | taaaaataaa |
| 88141 | aatggaaaaa | ccaataacaa | acaagcaatt | ccagccaact | ctttttcctc | tgctcaagat |
| 88201 | gaaatgaaga | aagtgagcat | ggtatgtaac | aactaatggt | aattttccca | gccttaatag |
| 88261 | tctacaaatg | atcacagata | agggatatgt | acatatccta | atttattccc | ctgacctaaa |
| 88321 | cattgctctc | ctaacagtca | cctcttctga | acctgctgct | ctctctgata | tgacaatcac |
| 88381 | ttgtaaatct | tgaaagacag | ttaagccgtg | attgtaattt | tttatacata | gtatctaaca |
| 88441 | cattaggaat | gcagagtaaa | tgccaagcta | taatcattta | aatgtccttc | tttatttatt |
| 88501 | tcatttctca | tgttctgtgt | ttgaatgaag | actcatggaa | acccttgttt | tcgcatttaa |
| 88561 | tcacgcagtg | ccaggtggcc | ccagccccag | gctcaagaca | accattagca | ggtttttctg |
| 88621 | ctagggtgga | ttcatcctct | atatacattt | ctgattgtct | ataaatagca | tcacttgtga |
| 88681 | ggattgttga | aataatttgt | tgctataatt | atagtgacat | ttctagcatg | ttataatcat |
| 88741 | ataaaactgc | caaaatagga | ggatgctatt | ttggactttt | tttttccagt | ttagtttgtg |
| 88801 | tttcatggaa | tggaagaaaa | gatgaggaat | gcctctggtt | tagagaagga | gccacattat |
| 88861 | aacagaagag | aacaatgaca | cacaaaaatc | aggagtactt | ccttcagaag | aaaggcagct |
| 88921 | ggagcctttg | aaattgacat | agttttagtc | aaaaagaaga | aaagaaagat | ctccaaaata |
| 88981 | tacaattcca | tacatgcaag | agaagtgaag | acatgtgatg | atatttttcc | agctgaaggc |
| 89041 | cttagccaaa | ccaaagtcat | tttaattttt | cataggcttt | tacatttttg | atgacaacat |
| 89101 | cttggttgag | gaatcacaaa | tgtctgaaaa | agaagtcgca | aggatggttg | cactgtgaaa |
| 89161 | atctttgaaa | aagtcagtat | gttttgaatt | gacattgtat | ctccactgtt | ttcaaatgtt |
| 89221 | cagccaatgc | caattaaaca | aaataaagat | gtatgttgta | ttagttcatt | tttatgctgc |
| 89281 | tgataaagac | atacccaaga | ctgggtaatt | tataaggaaa | aagaggttta | atggactcac |
| 89341 | agttccatgt | ggctagaaag | gcctcacaat | catggcagaa | ggcaaaatgc | aatttacgtg |
| 89401 | gcagcaggca | agagagagaa | atgagagcca | agtgaaaggg | gtttcccttt | atgaaactat |
| 89461 | cggatctcat | gagacttatt | caccattgtg | aaaacagtat | gggggaaacc | acccccatga |
| 89521 | ttcaattatc | tcccactggg | tccctcccac | aacacgtggg | aattatggga | gctacaattc |
| 89581 | aagatgagat | ttgggtgagg | acatagccaa | accatatcat | atgtattgat | aaaatgctgt |
| 89641 | tagctagtga | acaatcacat | actttgagga | ctgtaccaca | gttgtggatg | ttttcaaact |
| 89701 | aaaatggcat | actgtatcta | cataagttgt | gttgatggac | aaaattagaa | aatgtcaatg |
| 89761 | tttattttag | atgcaatttc | tcttttttct | tagtgcccag | tgctgggacc | tactcatttg |
| 89821 | acctgcctga | tgacactaaa | caacaaagtg | ggaattggct | ttcttggcat | taatcaaggt |
| 89881 | tataatttta | tatgtatttg | taggatcttt | cccccacatc | tgtacactcc | aagaaggtgc |
| 89941 | agatcctgtc | tggttggaac | tcagtcatat | tttcagcaag | gtgacaagtc | ataggtcatt |
| 90001 | ttgaggtgaa | cgaatgaatg | gtggaacagg | aagacttcca | aataatcctt | gctctgaaat |
| 90061 | ttacaagttt | tataagctca | ggccagcatt | tcccttcttg | gagcctcaga | ttcattcttt |
| 90121 | acaaaatggc | tgatttggct | tggaggctct | ccaaggtcca | tcttaagttc | cagaagcctt |
| 90181 | caattccata | aatgaatata | cagtagatgc | cctagggctc | taccaccgcc | tctcttctga |
| 90241 | aaactgtttg | cacaacccgc | atctccgtct | cactgcagca | caacattccc | tggagatagt |
| 90301 | catctaggcc | agagttagac | cccagatcct | agctggacgg | agcacctgct | tttttccagg |
| 90361 | tatttggtgc | taggcctgag | gacctgccat | ttggaatctg | cttacagctg | ctctgtgacc |
| 90421 | tgcggatttg | gggaccctgg | gagtgactgc | ctcctgccca | gtgtggaggg | ctgggcagag |
| 90481 | acagccagaa | ggcagagaga | gaggaataaa | gccgtctcca | gaccagagcg | gacagagcaa |
| 90541 | gtggagactt | ctggcagttg | ccatcccttg | ctgaaagctg | agggttccat | aagggagctt |
| 90601 | gtatactcat | agtgaatttc | ctttttgccc | tttttttgga | tgaaaagtag | ctctaattag |
| 90661 | ctttgttttg | tatccagaaa | cccaactaat | aacatacatc | cattcttttc | agtagttaaa |
| 90721 | cctagtttaa | ctggtgattc | tggtgaagtc | tcctagactt | ttcaaagcct | cattcataga |
| 90781 | agcctaaacc | cagtatttgt | tgtgaacagc | attatcagtg | cctgtgaggc | agtctctctc |
| 90841 | tggggtgaac | cgcaatttag | gcagtctttt | gtgtttctac | acagcaggaa | ccttataaaa |
| 90901 | ctggagaggc | aagagtcttt | gacctaggat | gagtccatag | cccatttctt | tgccccattt |
| 90961 | ttaaacaggt | tggtcttgcc | tcctgttgtt | gatctttgag | tcctcccctg | cacacctgcc |
| 91021 | tggaatgctc | agctcttcct | gctcacctcc | gcagacttcc | agagcctgga | cagaagtcac |
| 91081 | ttccctgaca | gatatcagca | caaatctctg | tctacctttg | gtctcctcca | aactgccctg |
| 91141 | tcttcttgtc | acgcacatcc | taacttcata | tttcctttcc | tgatctcatt | gagaaatgag |
| 91201 | aaatcgggtt | atgggttatc | taattccatt | agcctcatta | ttccttaggc | atctcagtcc |
| 91261 | cggagagcta | atgccatgtc | atagaaggag | tcggaagact | cggagtccac | cagtaactct |
| 91321 | gtgatcttga | acaaaacccc | ttctctcttg | atttgtaaaa | tgagacctaa | attgcataat |
| 91381 | ttgtgggata | cttccatctt | cactctaggg | tctatttcag | ataaaatccc | ctccccactc |
| 91441 | tcagttctca | tctcaatttc | tttctttttt | catttttatt | ttattttatt | tttagacgga |
| 91501 | gtttcactct | tgttgcccag | gctggagcgc | agtggtgcga | tctcagctca | ctgcaacctc |
| 91561 | cgcctccctg | gtttaagtga | ttctcctgcc | tcagcctcct | gagtagctgg | gattacaagt |
| 91621 | gcccgccacc | acacccagct | aatttttgta | tttttagtag | agacggggtt | tcgccatgtt |
| 91681 | ggccagggtt | ctctcgaact | cctgacctca | ggtgatcctc | ccgtctcggc | ctcccaaagt |
| 91741 | gctgggatta | caggcgtaat | ccactgtgcc | cagcctcatc | ttaatttcta | ggttcctacc |
| 91801 | actccctttc | ttggcatgaa | ggggtggggg | tcttgtttgc | aggtggaact | tttccactgt |
| 91861 | gggtctctcg | gttttattgt | agatgtttta | aagtctcaac | taaggttcct | gacaggcaga |
| 91921 | gggaagatgg | gatgggatgg | gggaacagtg | ctcttcaacc | tggaggggcc | tggagggctc |
| 91981 | aggccactca | atacgtgcgt | tagtatattt | tcccacattc | ttgttcctgt | ctctttcctt |
| 92041 | gttaggcaac | accaccctgg | tctttttttt | tttttttttt | tcttgcctta | tctttgttaa |
| 92101 | gctttccaca | ttcctactcc | atcagcatgc | ttttggctga | caaacttgaa | gctttcccct |
| 92161 | tttgcttgct | ttgcagctat | catctaatct | tgcaagagat | gagaattctt | gacatgtttt |
| 92221 | tgttaacttc | ttttgttgcc | actatgagct | ccacctaatt | ctgagactga | catcccccaa |
| 92281 | gcagcaggca | gtgaggcaag | gctctggggt | ggctggcctg | gccttgccac | tctgaggcca |
| 92341 | gtattaaaac | actgagtggt | tggtttcatt | ccctacatta | ccaacttttt | cttttttctt |
| 92401 | ttatttcttt | ttctttttct | ttcttttctt | tttttttttt | tttgagacgg | agtcttgctc |
| 92461 | tgtcacccag | gctggagtgc | agtggcgtga | tctcggctca | ctgcaagctc | cacctcccga |
| 92521 | attcacacca | ttctcctgcc | tcagcctccc | gagtagctgg | gactacacgt | gcccaccacc |
| 92581 | aagcctggct | aatgttttgt | atttttagta | gagacaaggt | ttcaccgtgt | tagccaggat |
| 92641 | ggtctcgatc | tcctgacctc | gtgatccgcc | cacctcagcc | tcacaaagtg | ctgggattac |
| 92701 | aggcgtgagc | caccgcacct | ggctttcttt | tatttatttt | tctaatctaa | attttaaatt |
| 92761 | atttaaaaca | ggatctctct | ctgtcaccca | gggtggagtg | cagtggcatg | atcatagctc |
| 92821 | actacagtct | ccatctctta | ggcttaagtg | atcctctcgc | ctcagcctcc | cgagtagctg |
| 92881 | ggactacagg | catgtgctac | cgcactcagc | tgattttatt | tagttttgta | gagatgggtc |
| 92941 | tcactacatt | gcccaggctg | gtctcgagct | cctggcctca | agcaagtctc | ctgcctggcc |
| 93001 | ctcccaaagt | gctggaaatc | cgggaatgaa | ccatcatgct | cagctttgct | gtcttaatga |
| 93061 | gagccaattt | agccactctg | gctgaaaccc | ttctcttcat | atgacagccc | agccatcaaa |
| 93121 | catcagttgg | atccaatagt | tgagtatgtg | ggcttcttca | gtttcctagg | catggctggg |
| 93181 | tgaccagaga | aagagaagtg | cccactcaac | tgtccaccaa | tttggaattc | tatcattcat |
| 93241 | aactagcatt | tatattacgg | cagattttga | gcgataaagg | tgatcacaca | attgcaaaac |
| 93301 | atgctttctg | tatatttatg | accagggttt | tttcatgcct | ctctcactga | cttccacccc |
| 93361 | tggtccttgt | caagcttcta | agagtttctc | agtgcctttt | cccccagagc | ttagatcata |
| 93421 | tccaaggaaa | gattgaaaat | aaagtaattc | actaagtgga | aaagaggaga | tttttttatg |
| 93481 | actatcagtt | tgcaaaaaca | aaattaaaga | taatacaggt | tcatgcaatg | tcacggataa |
| 93541 | tgacaacaat | ctagataaca | gccaagaaaa | ggcaacttta | ttgctttctt | gcaaacaagc |
| 93601 | caagaacaat | agttcatcaa | gtttccacta | tattcataat | ggagcatttt | atgatcttct |
| 93661 | aaggagaagt | ttgatgaaat | gcaagactgt | gctttagata | aaaatgtcaa | ttttcattaa |
| 93721 | aatctttaag | tttatttgtc | acactttctt | tctaaattcc | aaactaattt | ctttggggct |
| 93781 | catgtctcta | ttaatttgct | tgtttctttt | gtaacataaa | agctgtgaca | tttagattta |
| 93841 | aaatgaccat | tgtttctgca | ctggatgttc | ttggcctgtt | atttccttca | taatttttac |
| 93901 | tacggctttt | ggggattcat | tcacttagtc | ttggcagtta | ctgaatcttc | ttgtcctttg |
| 93961 | cagctataga | gatgcctcaa | gttggcaaga | aagaaaagga | agtctttgtg | ccgttgaata |
| 94021 | ccaaatctgg | tctctattac | agtgccgaag | gagtgctgtt | tggaatgaca | ttggaaacag |
| 94081 | ccaaagaaca | tctccaagac | cttctgacgt | tgatggagag | aacctctgac | tgaatccttg |
| 94141 | gtggccaggc | aggcagggca | gctcattctg | aatgagtaga | aatctcattt | ataatgttca |
| 94201 | acctaggtaa | catgttttag | aagacaactt | aagtcagagg | gaaagcctat | tatggccaaa |
| 94261 | gcctgaatat | ttgtccagtc | ggctgttaat | ccagtcagct | ttgatggaag | aattggtaat |
| 94321 | aggaagattg | ttctttttgg | cttgttgggc | tggagtcaga | gagaggggga | cacccacatg |
| 94381 | gccagaaagc | tgagatgtca | tagctagata | gtcctcgcaa | atgccacttt | accaaacagc |
| 94441 | cttccattat | gattgtctgc | aaatgaaagt | taacactgct | acacctggca | aggaaattct |
| 94501 | ttgcagagcc | atgggtgtag | ttaccacact | gcactgcagc | agtttgggag | aagatgctga |
| 94561 | attagggtga | acctccagta | gcctcttctg | ggcttcttca | tggaaaggaa | gtgagagagc |
| 94621 | ttcctgaagc | aggagatggg | acaggtgcca | ggcaacagaa | gaaaatcagg | ccctgcacaa |
| 94681 | tggatcacgc | ctgtaatcct | atggcttttg | gaggctgagg | caggaggatt | gcttgagctc |
| 94741 | aggagtttgg | gaccccatct | atacaaaaaa | aaattggcca | ggtgtggggt | gacatgcatc |
| 94801 | tgtagtccca | gctactcagg | aggctgaggc | aggaggatga | cttgagccca | ggagttcgag |
| 94861 | attgcatcac | tgcactccag | cctgggtgac | agagccagac | tctgtctcaa | aataaataaa |
| 94921 | gaaaagtcaa | aaaaagaaaa | tcagcgattt | ggccctacca | ggccaggatg | caggaaccct |
| 94981 | ggctgggaac | ccaagaggca | gtgggaaaag | caatgttagg | gttccaagcc | cttgtggagt |
| 95041 | gagtgctctg | ctccactgct | ggtctgagcc | aaccccctgt | gttcatggca | cctctacttt |
| 95101 | gcgttctgag | agcttgggct | tggcagtgac | ctggatgagg | cagaaaaagg | gagacaggat |
| 95161 | tgtgggacag | gcacagaatc | tggagacagg | gctactattt | gctttctttc | gagggaggag |
| 95221 | cacaaaagcc | aaatatgaag | tccttccttc | caatcttcca | ttgtctgaat | tgagctgact |
| 95281 | cactgcagag | taaactcatg | gatagccttg | gtttttccag | catgaacagc | tgggggcaac |
| 95341 | ttcacaaggg | tgtgagattg | gggagctaga | atggtgcctc | cagtgtgtgt | gaagacactg |
| 95401 | gagagtcata | tttacaccgt | cacgagctct | cctcccagaa | aatcatcacg | tttattatct |
| 95461 | tgccaattaa | acctgcataa | atgcatgggc | aaggtaggag | aaaaatggaa | aaaatgcaca |
| 95521 | gttcatttcc | ttctctggtg | ctgtttactc | tacatgagat | attattaaaa | gagaaaggtg |
| 95581 | gtaaacaggc | agaaaactgc | cctgtaacac | ccacaggtca | accaaaaaag | tctggtgtga |
| 95641 | ttttaatctc | agcaattttt | gaaatttagt | gatgaagaga | aatgctgaac | cccctttctg |
| 95701 | gcgtgttaaa | taatgataat | gtatgcttta | aaaactcatt | acaaccatta | acatgaataa |
| 95761 | ctagtgattt | caaatttgag | ttaaatagtt | aggctgctac | atttatgttc | tttgggtgtt |
| 95821 | tgaatataat | tatttgagga | ttaaattgag | acacatcgat | cggtcccaga | aataaataac |
| 95881 | aggcttgctt | gcaaagccaa | aattggaatg | gagaaatgag | aattcttaga | gaaaatatct |
| 95941 | gttgtatgaa | tgaatgaata | aaaaggagat | attccacaca | ttaattacag | gaagatttca |
| 96001 | ggaaaggatt | tggttgaagt | ttttaagacg | atgatgaccg | taatagatga | ttttaagaaa |
| 96061 | cacttttgat | tttgtttctt | gtttttatga | tttctaagat | tggagaagga | atttattaaa |
| 96121 | aggacaaaaa | tgtactttta | ttatttagta | atcttcttga | tttggacaaa | aactgatgag |
| 96181 | aggctacctt | tcttgaaata | aaaggagtat | ctaggatgca | tttaaacatt | atctcaagta |
| 96241 | tcagttactt | agatattaaa | tcaaaacaaa | cccactgcag | tatccttcct | tcctaataga |
| 96301 | caaatcctta | gtaatttata | ttgtatccag | agaaagtgga | atttatcatc | aaagctaagc |
| 96361 | aaatcagtaa | ctctttgttt | cgttacagag | gtaaccttgg | atttggtggt | gttgaatgta |
| 96421 | tttaaacctc | aagatcagtt | ggctacatat | attttaggaa | aaaagaaaat | cctttcaatt |
| 96481 | aaaatttttt | acagtgtaaa | tgtgttttga | gtgatttaag | ttggaccccg | aaggttgatt |
| 96541 | ttaatgtact | ttttttatta | aggatttcat | tgatgtacaa | gttgaggata | cttttggctg |
| 96601 | aaagtgctat | tgagatgtta | ggaaaatatc | aagcaaggtg | agcaaaatag | ccaacttgca |
| 96661 | agcagtattc | agaaggtctc | accaaaataa | aatcatcaaa | gaggagcttt | gaaatgcaat |
| 96721 | gtggaacgga | atgcaggagg | cattttcaaa | ggctttctgc | ctttggactc | acagaccacc |
| 96781 | aaaagattag | caagattctg | atgtacagga | atgttaggaa | gtttctgggc | tcactcatcc |
| 96841 | ctcagagact | taaaaagatg | acaaaaattc | aaaccgtcaa | gccattttgt | aatataaaaa |
| 96901 | agaatccaat | ccatccatgc | ttgtaatttc | cataaattag | ccttgtagac | cagcatggtg |
| 96961 | ttttgtaaca | tcgtggagca | atgcattata | caaattagac | aagggctctg | gccaagaaca |
| 97021 | gttttaagga | aaatgacaga | tcctttgctg | tttctcgcac | actgagctca | gatcttctct |
| 97081 | gtctgactag | aatcacaaag | aggaaactga | atattttatc | caaaatatta | ttataaaaca |
| 97141 | gtgctacaaa | ggcccttccc | aagagacaga | tcttgcagca | aatatcagca | aggcaggagg |
| 97201 | aaggagaggg | tcctcatagc | ctcctgtctc | tgtcagtaac | aggcagtcgg | taggacttgg |
| 97261 | actccgaagg | catgggctgg | atttgcaaat | tgcattttca | aaggagcagg | gagatacaat |
| 97321 | taggaagatc | aatgtatgaa | aacagcacct | gaggctgctg | gagtggaaat | gcaaaattgt |
| 97381 | gctgaggatt | caagaaccgt | tctgacatgg | ctgttagaga | cgctgaaatg | tgatttaaaa |
| 97441 | aaaaaaaaat | ttgccatttc | tccaccagaa | cccccagctc | ttgccaagaa | atcacctctc |
| 97501 | tgagcttcta | tttcctccct | ttgactctcc | cctcttcctc | atcctcattc | tcacctccat |
| 97561 | gcctttgaat | actgtttgcc | tcacctagac | catctattcc | ccatgccttc | ggatgaattc |
| 97621 | taacaatcaa | caactattca | ctatatgctc | tgtgcaagac | attctgcttg | acatttagag |
| 97681 | aattcagaga | tctccaagac | ccaggcactt | cccttaagat | gttgaaaatc | cccttaaaag |
| 97741 | gataaaaaac | caaatgtata | agagataact | agaaacagac | acataaaatc | tccaaatgtg |
| 97801 | cgggagtgag | acagaaattc | agaagagaga | cattatcacc | tgggaagtca | aggagacttc |
| 97861 | atggaggtga | attgaactaa | gccctgaagg | caaagcagga | ttcaggtagg | tagaatcaga |
| 97921 | tagggctggg | gatgtgggct | ggcatttccc | gcaggagctc | gaagcagcat | aggtaggcag |
| 97981 | gaaggatgaa | gaaaagagat | tgggagagat | ggcgttagag | aggttcaggt | gtctggagca |
| 98041 | gtgagtaact | tacttgcagg | gctaatggac | tagcattttt | acattttgct | aggttacgta |
| 98101 | gtaagtgaaa | gggagttgct | agaatgaagg | ggtgttttag | gaataattta | caggacccag |
| 98161 | tgaaacatga | aaaacagggt | cccttcttca | aaaaggaggg | aaaaaatttc | cattaagttc |
| 98221 | ctaaaataga | aagtcaatat | cttttttctt | ctgcagtctc | tctccaccta | tcactttgtt |
| 98281 | gttggttttg | ttttgtttcc | ctatttaatg | tcaatttcct | tgggcacaag | acgcttgcag |
| 98341 | gaaaagtgca | gtctcccatg | ggtacctggg | gaccccaccc | tgccctagtc | cagtgtgctt |
| 98401 | tgtcctggcc | gaccatgggg | aagttgagcc | aggcatcttc | cctccccaag | gtgtgctgct |
| 98461 | gcaacccatg | gcagatgagt | gacctcaagg | gtgttgcact | tggatgtgct | gggtacttgg |
| 98521 | attgggtcta | gtaaaaggct | tacccctgcc | aagttgcctg | tccagggctc | tgtgatacag |
| 98581 | gcagcccagg | tcagggatgg | cagaagccct | gagactccgt | agggcacgtg | tccttcccga |
| 98641 | agcctgtgcc | tacccccgct | gagagaggag | ggcagtggct | attgctgcag | aagcaaggaa |
| 98701 | gaggaggtca | gtggaatgca | gaaaaccaag | cacaggggtc | cagcaaccca | tgagaggaga |
| 98761 | cagaaagctg | cagggaggca | gggttgcatg | tgagctgagg | ctccaagtcc | cagtgcatgc |
| 98821 | tccagtgtcc | caccagactt | cagtcacaaa | gcacaaggtc | aaggtaaaat | tgttcaggat |
| 98881 | tccaaggcag | tgatcacaaa | gcattaaacc | ccaaatgcct | gagtttgagg | tcacctgtga |
| 98941 | ctgtagtggc | tgcatgctgt | caagctggcc | tttggggcag | cagagagacc | tgggaaatgt |
| 99001 | caatgccctg | gcacgggcgt | ctgggtgtga | cgcaggggta | gcaatggagt | gggaaagaac |
| 99061 | ggaaccataa | aagcaaggaa | gggaagtctg | tggactggaa | agaaaaataa | agcaaagaca |
| 99121 | attattgcat | ttgggacttg | ggtgatacag | aaaaagacga | cactctaata | taaaaacgaa |
| 99181 | acgaagcaga | agtcgtttta | tttctgttac | aattttagct | ttgtaggtag | ggttggagga |
| 99241 | ggggcaaaga | tacgatgttt | cattttggac | ataacaagtc | tgcgttggga | gaagttactt |
| 99301 | ggacgaagtg | tctgataggc | aggtgaatac | accccaggga | acttgaggga | aaggtaggga |
| 99361 | ccaagacaaa | gatgctgcag | aaatttgccc | agaaaggagc | ttccaagaag | tagatgaggt |
| 99421 | ctttaagaga | gaaggaacaa | ggaggggtga | gcagagatca | gagaataggc | tcacagggta |
| 99481 | cctgccttta | aaggacagag | agaaaggggc | ttccaggggg | aagaggagga | tctgcagaga |
| 99541 | ggaaactctt | gcataaggct | aagagaagag | tgaatcttaa | aaaggatgtc | aatggagtca |
| 99601 | aatagcccag | aaaatcctgg | agaatgagga | caagaaaata | attcttattt | ataattccca |
| 99661 | aaggtattgt | ttcagtggca | taattaagtg | gctgactgtg | tgcatgggag | ggctgtatgc |
| 99721 | aaatcatgac | tctgtggctg | agttgtctag | gatggacaga | gcagaagggc | tttagaagca |
| 99781 | gggtccagat | tcaaatctca | atttggactc | ttgctagttg | catggtgttg | ggcaagctac |
| 99841 | ctaatcttgc | taaatgccat | ttttctgagc | tgtgaaatga | gtaggtaagt | aggatattca |
| 99901 | tatgggagct | atcatacctg | gttaattttt | tgtagagatg | gagtcttgct | atgttgccca |
| 99961 | ggctggtctt | gaatgcctga | gctcaagtga | tcttcccacc | tcagcctccc | aaagtgctag |
| 100021 | gattacaggt | atgagccacc | atgatcagtc | aaatacacat | tctttttttt | tttttttttt |
| 100081 | tttttgacag | agtcttgctc | tgttgccagg | ctggagtgca | gtgacatgat | ctcagctcac |
| 100141 | tgcaatctct | gcctctgggg | ttcaagtgat | tctcctgcct | cagcctccca | agtagctggg |
| 100201 | attactggtg | cgcaccacca | cactgagcta | atgtttgtat | ttttagtaga | gacggggttt |
| 100261 | taccatgttg | gccaggatgg | tctctatctc | ctgaccttgg | aaacacacat | tctttaaact |
| 100321 | aggcacatga | ctgctctgct | aagatgataa | ttcccagcct | cccttgatga | tagatgtggg |
| 100381 | catgtgacaa | agttctggct | tatgggagat | gaaataaagg | gatatgggca | agtttctgtt |
| 100441 | cttcctttat | gtttcccctt | cctgatggcc | agaacccaca | tggatggctg | gaactgattc |
| 100501 | agcaatcttg | gaccaggcaa | cagaagtggt | gggttgaggc | taatgtagaa | aaaagacaga |
| 100561 | aattgcctga | atctgctacc | ttgtggggct | tccaaccttg | cctgtatttg | gactattaaa |
| 100621 | tgagagaaat | actttttttt | ttaaatttaa | gtcactggca | ttttggctcc | ctctatataa |
| 100681 | ctgcacttgt | atcctaagta | ataatatact | tgtgcacatt | taaaaatcgg | ggtataatac |
| 100741 | ttacttcata | caatcgtaag | aactgagccc | tctctgatcc | ataaaaaaca | ccagacccac |
| 100801 | tacctacctc | attgtaatta | cttaatcaat | gttttctgaa | tacatgaaca | ttatgaccag |
| 100861 | aggacacggc | tgagagaatt | gccaaggtat | gtatgagaaa | tagtagtggg | agttacattg |
| 100921 | ttggctacat | tgtagataga | tgttaaagga | tttgaatttt | attctgcagg | tagtagctcc |
| 100981 | tggtggttag | taacatacag | aaattgagga | catgcccatg | cattttgata | caaatggaaa |
| 101041 | attcaatatt | tcttgagctc | ctatttgatg | ccaagcattg | ggaaaacaaa | ggtaaataaa |
| 101101 | ccagtctttg | tccttaagct | tcttgctcaa | agcaatcatt | atattgttta | aaagcttgga |
| 101161 | ctctagatcc | agaatgtctg | cttttgaatc | ttggctctac | tactagctag | ttggtgacct |
| 101221 | tagcaaatca | attcacttct | ctttgtttca | ttctctgtaa | aacagcaatg | acaataacat |
| 101281 | caactttata | ggattgttat | aaggaatata | tgagctaata | cctgcaaaat | gcataaaaca |
| 101341 | atgcctaata | ttaagtgctc | agtttctgct | agctattata | tcagataact | attgctacat |
| 101401 | aagaaaccac | ccttaactcg | gtagcttaaa | acaacaaaca | tttatttagt | tcacaattct |
| 101461 | tcaggctggc | aatttagcct | gggcttagct | gtgtggttcg | tctggtcttg | gttgtgctcc |
| 101521 | agtatatgtc | tgcagttagc | tacaggttgg | ctaggtggct | ctgctttcag | ggttagtgga |
| 101581 | tttctacggt | catagctatg | gggtgacagt | gacaactaag | tcatttgtct | ctcatactct |
| 101641 | agcatacttg | tcaactttgt | tctcatagag | gaggcaggat | tccaagagaa | agtagaagtg |
| 101701 | tgaaaggtct | caagtcctaa | gcacagaact | ggcatagcat | tactccctcc | atatgatatt |
| 101761 | ggtaaaagca | attagcaaat | tcaaccaaat | ccaagggaag | agcaacaaag | tcacattgca |
| 101821 | aaggatgtgg | gcacaggttc | aacaggtgta | attaatttgt | ctcagtctgt | cctctggtct |
| 101881 | taattattca | catcccttct | acatgcaaaa | tatgctcgca | ccctcccaag | accctcaaac |
| 101941 | tctcattcaa | tcatggcatc | aaactccata | tcaattctgc | acatggttcc | ttggatgtga |
| 102001 | tgcgtcttga | tctggggcct | gtgaaatata | gagtcaagtt | agtacttgtt | cagaaagacc |
| 102061 | caacatataa | tgatgacaca | aggcacagga | caagtgtggt | aaatactccc | attaaaaatg |
| 102121 | aggaggaata | ggagacacta | gcagtaactg | gtccatagca | attctgaaat | cccactgggt |
| 102181 | aaatgtcacc | agatccccca | actgtgggag | tgggaaaaag | ggagtgttgg | ttccctaatt |
| 102241 | aaaccttgct | tctgatcctt | ggaagtggtt | ctttggtcta | tttttctaca | aatttcttct |
| 102301 | tccaccttgt | ggtttcctag | ttgtgccccc | tgagatggtt | ttcttttctc | atcactcttc |
| 102361 | ttggctacat | ttaaataaat | agcccatgtt | agcagataaa | tgtctgtctt | aacttactta |
| 102421 | tgcctattga | aagtgggaag | cccaaagccc | tttgtacatt | ttgaacaatt | tcagcccctt |
| 102481 | ttagtccaag | ctggccacat | gtttttcctg | tacaatttcc | taaacaatgt | agtgggtttc |
| 102541 | ttactaatct | gattatttta | cccacttcaa | tggggcagaa | gccaacacca | caattgtttt |
| 102601 | cttgacctgg | ctctctagat | ttacctactg | ctacattgag | actaggtttt | tgtgaaatta |
| 102661 | tgacattaat | attctagaag | tctttggtcc | agaccagatg | gtctacttta | attatttttg |
| 102721 | aggcctcaac | aagagatcat | acagccacac | ccttgatttg | atctatgtct | tagtctatat |
| 102781 | tttactttga | gaatcttctg | ctggctgaag | agactacaaa | tgagtaactg | ttatattttc |
| 102841 | taatccagca | agtcattcta | tttacaaatt | ggccagttct | ttctttatct | catctttcct |
| 102901 | tggtgttcgt | tgtcatatgc | agcaagaagc | aaccagctaa | cactttcaac | attttgcctg |
| 102961 | gaatctcctt | agccaaatgc | aaacatttgt | taggcatatt | tttatcttcc | tatcttatat |
| 103021 | ctctatctct | gtattgaaat | atctatatat | cttgctgcag | gtgagaatta | tctttgaaat |
| 103081 | aacagcatta | ttgagaaata | tttcatatac | tataaaattc | actgtttaaa | ttgtacaatt |
| 103141 | cagtggttgt | ccaaccatca | ccactatcta | acttcataaa | atttttgttt | ttccaaaaac |
| 103201 | gaattctgta | cccattggta | gtcactccac | actactccct | tccttgggcc | tctgacaaat |
| 103261 | atttatctaa | tttctgtttc | tattgattag | cctattctga | ccatttcata | taaatgaaga |
| 103321 | tttacaatct | atgggctttt | gtgactagct | tctgtcagtt | agcataatgt | gcagggccaa |
| 103381 | cagttttaca | agtccttttg | ccgttacata | aagcaggttg | ccattttttt | agctttaaaa |
| 103441 | acaatttcct | caccatttct | ccagcctttg | ctgataatgt | gcttatggcc | tctccatcct |
| 103501 | ccaccctctg | actggttcta | aaatcaatgc | tgcacatttt | caatcttggt | tatgtcagca |
| 103561 | ttccatttct | ggtaccaatt | tcaggatcag | gatcattctt | tattaccaaa | atcatgttgt |
| 103621 | ttattaaaaa | acaaacttca | gtggcatgca | ataataatca | tttagttttt | tttcatgagc |
| 103681 | ctacaggtca | gttgggtggt | ccttctgggt | gtgattggct | caactatgta | tctgatgtta |
| 103741 | acttcacatc | agattgaatt | attctgacct | ctcttgggtg | ttgactcact | gtgggcttat |
| 103801 | ctgtcatggc | ctcacaagga | gagctgggct | ctccttcatg | tggtctcttg | gcctccatag |
| 103861 | gccagcctgg | ctccttcacg | tagtggctgc | gcagagcccc | aagagaggaa | gtggaagcac |
| 103921 | atgaggcctc | tcaaggccta | gagtcagaac | caacatgctc | gcttctgccc | tattctattt |
| 103981 | gccaaagcaa | atcacaaggc | cagctgagta | tacacaagca | catgcatgca | cacacataca |
| 104041 | cgcacacaca | cacagaaaga | gagatttatt | ataagaaatt | gactcacatg | accatggagg |
| 104101 | ctaagaagtc | ccgtcacctg | ctgtctgcca | gctggagact | cagaagagct | ggtggtacaa |
| 104161 | ttccagttct | actctgaagg | cctgagaacc | agaaaagctg | agggtgtaaa | ttccagtcta |
| 104221 | agtgcaggag | accaacgaca | cagctcaaaa | acagtctgac | agaaagagag | tgaatcctcc |
| 104281 | cttaccccac | ttttttctca | gtcccttaaa | tcaatagcct | acaagtgttg | ttgtaaagtt |
| 104341 | aaagttttta | aactttttat | tttgaaataa | ttgtagttgt | aagaaataat | acagagagat |
| 104401 | cctgtttacc | ttttatttgg | ttctgcctca | tgttaacatc | tcatataact | atactaaaat |
| 104461 | attacaagta | agaaattgat | attgatacac | tcagcatatg | tatatataaa | taaatatata |
| 104521 | ctatataaat | agatatattt | atagaaagag | aaattgatat | tgctacactc | tgcatatgta |
| 104581 | tttatatatt | atatattata | tatttatata | tgcagagtgt | agcaatatca | atttcttact |
| 104641 | tttgatatat | atataatcag | agtgtatcaa | tatcaatttc | ttacttgtga | tatatgtata |
| 104701 | tataatatat | atatacacac | agaaagaaag | agagagagag | aggagagaga | gagagagaga |
| 104761 | cagggtctcg | ctctgttgcc | aaggctagag | tgcagtagtg | caatgtcagc | ttactgcagc |
| 104821 | ctcaacctcc | caggctcaag | ctatcctcct | gcctcagtct | cccaggtagg | gaccacagac |
| 104881 | atgcacaacc | atgcctgcta | acttttgtat | ttcttgttag | aaacggggtt | tcaccacatt |
| 104941 | gcccaggatg | gtctcaaact | gctgagttca | agtgatcctc | ccgtcctggc | ctcccaaagt |
| 105001 | actgggatta | tagattcatg | cccatcccac | tctgccaatc | ttattctgat | ttctccactt |
| 105061 | ttacatgcac | atatttatgc | atatgtgtat | ttaattctgt | gccatatttt | cttatgtgta |
| 105121 | gacttgtgtg | accatcacta | cagtcaagat | atagaacagg | tccatcacaa | ggacacatta |
| 105181 | ggctaccttt | gatagccaca | gccatttccc | ttcccttccc | actccctagt | ccctggcaac |
| 105241 | taactactac | atgttcttca | tttctataat | ttgttttttt | caaaaatgtc | ttataaataa |
| 105301 | aatcatgcag | cttataacct | tttgatattg | gctctccttt | ttttgaataa | ctcccttgaa |
| 105361 | atccaaccaa | gttgtgtgta | tctgtggttt | aaagctcctt | tttcttgctg | agtggtaatt |
| 105421 | cacagtatag | atgtaccaca | gtctatttca | ccattcaccc | actcagaaac | atctgggcca |
| 105481 | ttcccaggtt | ttagctgtta | ggaataaagc | agcaacaaac | atttatacac | aggttatttt |
| 105541 | gtggacagaa | ctcttgattt | ctctggaata | aatatccagg | agcataattg | ctggatcttg |
| 105601 | ggatgagcat | gtgtttagtt | ttataacaaa | ccagcctaca | cttttccaga | ttggctgtgc |
| 105661 | cattttatat | tcccattggc | aatgtaggag | gtatccagtt | tctcagcatc | cttgtcagca |
| 105721 | tttgatattt | ttaatttctg | ccattcaaat | aggtgtgtag | tgatatctaa | tgtggcttta |
| 105781 | atttgcattt | ctttaatgac | taaagatgtg | gggtgtactt | tttatgtact | tatttgccat |
| 105841 | ttatatatcc | tcttgagtga | agtgcctgtg | tatgtctttt | gcccattttc | taatttggtt |
| 105901 | gttatattct | agcagttttt | cattcctagt | ctcttgttta | ttcatcttcc | tataaagtaa |
| 105961 | cctaaatctt | accttgtatt | taagagattt | ttctatggaa | gagaatgaaa | agtcttatta |
| 106021 | aatctccaat | acatttttag | atagcgtact | catactctgc | gtgataaatc | attttatcag |
| 106081 | gaaagcaaat | agaattggtc | ctgcccaata | gtttttttct | caaagtcact | gattatttcc |
| 106141 | tagtctcttg | tgatacctta | caggggctaa | agaggaatga | tttgcttaag | ctgatcttac |
| 106201 | acgtcctgtg | cttatgcaaa | aaagcttagt | gttaagcccc | actaaagttt | gttgtttgta |
| 106261 | catctctaaa | agaaacagtt | ggccaagcgc | ttaggtagca | tggcaggtag | ggaccacaat |
| 106321 | agtatagttg | tacagttgtc | tgtgtttgat | ttgtaaccag | caaaaccttc | tcactcagaa |
| 106381 | aaaaaaagag | cagctttcag | ctcctggtac | tgctgatctc | ctctggtgtt | ttctgttaaa |
| 106441 | catgtcctag | atttgctaat | tgtgtttttt | atttaatgag | ttcaagaggc | aggcatttat |
| 106501 | tgtatccctc | gctaataaat | cataactgct | gattcagcat | ttctacaaaa | tgttaaacaa |
| 106561 | caatacaatc | aagccttagt | aattagttaa | tttagggaaa | caggaagaat | tactggactc |
| 106621 | ttgctagaaa | ttatacgata | attttttttc | tgcttttcca | tgctaactta | tagtaacctc |
| 106681 | cctttccccc | tttgcactgg | tttccttcca | agcacaggat | ataacctata | acctaaagaa |
| 106741 | ttgtataagg | ctttgctact | ttttttgggg | ggtcacgctc | tgttgcccag | gcaggagtgc |
| 106801 | agtggtgtga | tcttggctca | ctgcaacttc | tgcctccctg | gttcaagcaa | tcatcccacc |
| 106861 | tcagcctccc | aagtagctga | gattaccagc | atgcaccacc | acacctggct | aatttttgta |
| 106921 | tttttagtag | atacggggtt | tctccatgct | ggccagggtg | gtctcgaact | cctgacctcc |
| 106981 | agtaatccac | ctgcctcagc | ctcccaaagt | gctgggatta | caggtgtgag | ccaccatgct |
| 107041 | ttgcctactt | atttttttaa | acaaaaataa | tcaaataaat | atacattata | tacattgcaa |
| 107101 | attagtaaca | agtcttgtgg | aagaataaac | acatatttta | aaatgctata | gtaaaaaata |
| 107161 | ccccacagag | caatgcatga | agtgggttgc | aataaatacc | aaattcagaa | atcagcttgt |
| 107221 | gtcaaaagaa | atgatttaag | agagctgaca | cgcactgaaa | tatggactag | tctagagaat |
| 107281 | gcttgaagaa | gggaatcaaa | gcaataaata | aataatcatt | tgtattattt | caccttcttt |
| 107341 | gtaagtgatt | aattgattaa | aggggagaca | gggttaagta | attcaagccc | ccagcatcaa |
| 107401 | tcagaggctg | aattctagct | tgtgagctac | aagctggtat | agagagctca | aaggattttt |
| 107461 | ctgggttgtt | tctcccaggt | cagtcatttc | tggctttgga | agctctaggg | ttgctgggtc |
| 107521 | tatgtgggca | tttgttcact | agtgaagctg | tcctggaatg | ggtccagcac | ctggagcagg |
| 107581 | ctagtctcct | aacatcctgc | gctgagcact | catgaacacc | atgtgggatg | catgtgtgag |
| 107641 | aagccctctt | ggtggcataa | tgacagatgc | tctgttcgag | agaggctagg | taaaggcaca |
| 107701 | tctaatgctg | aaaaactcgg | gagtatctct | cttttcattc | agcatgtatt | cattgagtgt |
| 107761 | cttccacagt | ctagctctgc | actacgtctg | caatacagga | atgaaaaatc | cctgctcgta |
| 107821 | aaaactcaca | tgggagagag | attaataacc | atcaactaca | ataggacaca | agtaaggctt |
| 107881 | atgatggcag | caagtacatg | gtattactcc | agtgcagagg | aggtctgctt | catcccggtg |
| 107941 | gtggtaatgg | ggtgggaggg | gtgaaggaag | cgcttctgaa | tgcggtgaat | tctgagatgg |
| 108001 | gttttgatgg | tgaaatagga | atgtgctcag | cagacatagt | gagggggcag | attatgcaag |
| 108061 | acaggtgcag | gtgcgagaga | gtgcaagtgg | ctcttgcaaa | gctaacagtg | gtttgtggca |
| 108121 | gggggcagga | ggtgagggct | gcagaggtgg | gcctgagcca | actgaagatg | ttattgatgg |
| 108181 | gttggatgca | gggcaatgac | ataatgagaa | aggcagaatg | aggggcaact | tttgccacat |
| 108241 | tccaccatcc | acttgctttc | ctagaaggga | agactgggga | ggccactgca | ttgcatttca |
| 108301 | gagactgttt | gtaaatgtat | gtatgtttat | ttatttattt | attttgggag | cttattaaaa |
| 108361 | gtatgtgtgt | ttatttattt | atttatttat | ttagggggct | tgaattactt | aaccctgtct |
| 108421 | caccctatca | cctaggctgg | agtatagtgg | cacaatcata | gctcactgca | gcctcaaatt |
| 108481 | cctgggctca | agcaatcctc | ctgcctcagc | ctcctgagta | gctgggacta | caggctaatt |
| 108541 | agtcatactc | agctaatttc | tctatttttt | ggagggatgg | ggtctcgctg | tgttgctcag |
| 108601 | gctggtctca | aacctcccaa | agtgctggga | ttacacgtat | gagccactat | gcctggccta |
| 108661 | tatatatatg | tatgtgtgtg | tgtgtgtgtg | tgtgtgtaat | atatatatat | atatatatat |
| 108721 | attttttttt | tactatcaaa | ttgctcctcc | tgagcccagt | ccattttctg | ggtacagggc |
| 108781 | tacagacaga | ccatctttga | aatgccagtg | gacagatgtt | atcacagggc | atgtttccac |
| 108841 | cgatgggtcc | atgcctcgca | cctggagcac | agcagtgagg | tggggaagtt | ggagaatatg |
| 108901 | agtgctgatg | tcttgtcctc | cttttcacag | ctgccgcgaa | tccctcagat | gaatcctatc |
| 108961 | ctcatccatg | catctgattt | ctacactcgg | ggtgggatag | agtttgagat | tggaaaataa |
| 109021 | tgcaagagct | ttacaccatg | acttatgagc | gctggctgag | gtatctggga | agatgtagct |
| 109081 | ggaacagatg | aagtctcaca | ggtgacactg | ctgtttctaa | atatttgaag | ggttttatac |
| 109141 | gtaacagaca | ggattagatt | ttttctgtat | ggttgtaaag | tatgcattgt | ccaattgagg |
| 109201 | gaaactatag | gaaaaactga | cttggttcac | aataaaaatt | attgtgccta | tcattcggtg |
| 109261 | agtgactaat | acgtgctgtc | atattatact | gagataatcg | aatcctttca | acaaccctga |
| 109321 | gaggtgcatg | cttttaacct | cattgtatag | atgcgcatcc | tcaggtgcct | ttaggtttca |
| 109381 | aacctatctg | cctgactctc | tctttccatt | gcattcaggt | tcacttgggg | aaaactattc |
| 109441 | tgtaatatgt | tttcccctga | tggagctttc | agaggtgaaa | tggaccgtgt | gctgggtgga |
| 109501 | taattctccc | tccacctgga | gatcactgcc | atgaatattg | taaaggggag | tcaaacattg |
| 109561 | cctgggctgt | tgaacccctc | agccttcaag | gcccggttta | aaggagagct | tctcagatct |
| 109621 | gctgtccttt | tataagtgag | ctggatattt | acactacatg | gcatgtgtat | tagcttctta |
| 109681 | ttgctgccgt | aacaaattgc | catagatcca | gaggcttaag | acagcacaat | ctatgctctt |
| 109741 | acagttctgg | gagtcagaag | tccactttca | ctgggctaaa | tcaaggtgtc | tgcagagctg |
| 109801 | tttctttctg | taggctctgg | gggaagaatt | tgcccttgcc | tttttccgtt | tctagaggct |
| 109861 | acctgtattc | cttagcttgt | ggtgccttca | cacatcttca | tagtgtgttg | ctccagcatc |
| 109921 | atatcacctt | ctcctcctct | gtagacaggt | ctccctttgc | ctgcctttta | gaaggattct |
| 109981 | tgtgatggca | ttaagcccac | ccacataatc | caggataata | tctccatctc | aagatccttg |
| 110041 | acttcatcac | atctgcaaat | cccttttacc | atgtaaggtg | acattgacag | gtctcaggaa |
| 110101 | ttaggacatg | gacatctttg | tgggacttta | cattcagcct | actatagcac | gcaaaggcta |
| 110161 | aagcatggat | atgtatagct | aggatggcga | gatcttccca | gacttaggaa | aattcactta |
| 110221 | aagaatgaat | tccaatctgc | atatgtcctt | cagatactca | gaacagattt | cattcaaatt |
| 110281 | gttctctagc | atacgcatta | ctagtaaaac | aaagaacagc | tccacagata | gaacaaaaaa |
| 110341 | agaacgctaa | cttatctttt | ctcacacagg | atggtagcat | tttaaaattc | agataaatag |
| 110401 | aatatgtgcc | cagcatcaaa | gtctcaggag | gtaaatgaaa | tgctgaagta | caaaaataac |
| 110461 | caagagaaac | tagtcacaaa | tcagacaaag | accaagcact | agcattgttt | tgcattactc |
| 110521 | caaactgcca | aactattgga | cttaggggac | ctggggaagg | gatgctcata | aagatagaga |
| 110581 | atttaatatt | agctctggaa | atgggatttg | ccacatcgct | aatccaactg | gcaaaaagta |
| 110641 | gcatgtctca | gagtccaaag | cctgataata | aagtatcacc | ctgcactgat | gtcagtttcc |
| 110701 | cccatcctgt | tttaggctga | cttctccagg | gagctatttg | agctctcaga | ttcaccatat |
| 110761 | aggaaactct | gaaaatggaa | tgcatgcaat | aagaatttgt | taactatcta | aataggtatg |
| 110821 | cattaccata | ttagacacta | agcgagatac | aaaaataagt | agaatacaaa | attctgtgcc |
| 110881 | tgaagggatt | ttaagataaa | caaaaagctc | cctgcaacaa | tctaaataat | agtggctcaa |
| 110941 | aatatcatca | tggtctctgt | gttctaagct | tccactcctg | cttgtcattt | atgcacccaa |
| 111001 | ttgaacaaag | ctatccgtaa | ataacaggta | tttgtcactc | agttgttgta | cattagctac |
| 111061 | tataggtaag | actgaagaaa | ataaaaaatg | gattgagcag | tagagcaaaa | caaataaaca |
| 111121 | aaacatgtat | tgagttcttt | ctgtgtgcta | agccttgggt | tcctgcagat | gaatagagca |
| 111181 | gggtccttgt | tttcaagact | ctgcagaaca | ttctttgcaa | gatctaccaa | gagatctagc |
| 111241 | tagaaaggac | caaaaacatg | ggtgaaacta | tcagatttta | attttttaaa | agctaagctc |
| 111301 | tgggttctga | atagagcatc | ttataagtag | acttttaaca | tggtcgaatt | atgtttctag |
| 111361 | atcttcataa | atctccccga | tttgaaaata | ctccttataa | aatgtatctc | ctctctgttc |
| 111421 | taaaattcta | taaaatataa | ttgaaaagca | cgagggtctc | atttaaaact | taaaactatg |
| 111481 | taagtgcaga | accaaaaact | atacaatgtt | attcaaatta | atcctgcaac | taaaatgaac |
| 111541 | tctgccatat | tacatgtata | agtgtgtaat | agcaataatt | atgaatactt | atgaactaaa |
| 111601 | tctgagagga | cttttttgtc | actttctgat | agtaactgac | taagtgataa | ataaataacc |
| 111661 | aaataatcag | cattatcatg | tgatatggtt | tggctgtgtc | cccgcctaaa | tctcaccttg |
| 111721 | aattgtaata | atccccatgt | gtcaagggca | gggccaggtg | gagataattg | aatcatgggt |
| 111781 | gtggtttccc | ccagattgaa | tcatgggtgt | ggtatccccc | atattgttct | catccctgta |
| 111841 | gtgaataggt | ctcacaagat | ctgatggttt | cataaagggg | agttcccctg | cacactctct |
| 111901 | ttcttgcctg | ctgccatgta | agacgttcct | ttgctcttcc | ttcatctttt | gccatgattg |
| 111961 | caaggcttcc | tcagccatgt | ggaactgtga | gtccattaaa | cctcttacct | ttatcaatta |
| 112021 | cccagtctca | ggtatgtctt | tattagcagc | atgagaacag | actaatacat | taggggccat |
| 112081 | ctgattttac | taactcgtta | agttgtgcct | ccaccaaatc | acaagttact | ataatccctg |
| 112141 | gaccttctaa | atggttcata | aaaagactaa | gccataatgt | tcaatttcca | tttgcagagg |
| 112201 | atctacagtg | cttgagaagt | tccaaggaga | actgagtgag | tgtgggctgg | agcattgaga |
| 112261 | gaacaggagg | gagctgggct | gagccttaaa | cgactcacaa | aggcgaaagg | agagtgttcc |
| 112321 | agtacagggc | acagcagagg | caaaggtctg | gcagcagatg | gcaatgacac | agttgagctt |
| 112381 | cctaaaagga | ccaaaggtgg | cattttggtg | agtagctgga | atggcagttg | aacagtggtc |
| 112441 | aggcaagtac | agtaggtaaa | ttccaaacat | ctagaaatga | gagattacat | tttttaaaaa |
| 112501 | ttttatttat | atttttggag | aggcagggtc | ttgctatgtt | gccccagttg | ctcttgaact |
| 112561 | cctggtcttg | agtgatcctc | ccacctcatt | ctctcagagt | gctgacatta | caggagtgag |
| 112621 | ccaccatgcc | cagccaagag | attaaacatt | aaaagaggaa | attaaaagag | tcaagctgaa |
| 112681 | gttctagtaa | ccatttccat | gaaccaaaaa | ttaaaaatac | aaaatggtaa | gaagtgtcaa |
| 112741 | agccatggat | gggacagagt | ctaggtcagg | cagcaggcaa | gggcatctgg | aattaaactc |
| 112801 | atatgggata | aaagggagaa | aataacattc | cagaggagat | ggtgaccaga | gacagtggtg |
| 112861 | gagggcaccc | tccatgctct | taaaaggacg | ctgaaaaaaa | attattgtca | actgctgcct |
| 112921 | ggaggctaca | gattatggca | actggagatt | catggaggta | ggaaagtaga | agcacagtgt |
| 112981 | gaagagtagg | aactgaccat | ccatccacca | gtgtcatcac | tccatctgtg | atagccccct |
| 113041 | ttccccaaag | taccagagct | ccaaatgacc | atgagaaggt | ctgtttccaa | acttggaacc |
| 113101 | caggtgcccc | gaagaggcag | ctggaaaatt | attgcaccag | gaagggtgag | gatatcgagg |
| 113161 | agaggaagat | tgagcaagtg | tgggctggag | cattgagaga | acagaaggag | ggagttaggc |
| 113221 | tgatccttaa | aagactcaca | aaagcaaaag | gacagaaaag | agagaaaaaa | atccatcaaa |
| 113281 | caagaagaac | cagcaaaaca | aaattccaaa | acacatggag | aaacctactg | ctaagagagg |
| 113341 | cagccaacaa | aataaaccat | caggacaaga | catcacatga | atgaagtccg | gacagagacc |
| 113401 | tacactgagg | atgctgaaaa | gagataaatg | aaagaataca | gctgtgaaca | gcaacaatga |
| 113461 | agcaaaagaa | ggtagaaaca | aaactagagt | ggatagatat | gagaaaaaaa | acaagtgcaa |
| 113521 | aattttgaaa | ttgagaaata | tagtaattga | aataaaaatg | aaatattcaa | aatacaggat |
| 113581 | aaactctaga | ctcaatcctg | tgggagagca | tatcagagaa | tgtgaagcta | tcactaagaa |
| 113641 | aatgctcggc | aagcagtgag | agatagaaag | atgaagatag | agacaggaga | tgagagagag |
| 113701 | attaaaaata | tatgtggtgg | ttcatgtctg | caacttggga | ggctgaggca | ggaggactgc |
| 113761 | ttgaggctgg | gaactagaga | caagcctggg | caacatagtg | agacagaaaa | tataaaaatt |
| 113821 | agctgggcat | ggtggtgtgc | cttagttcta | cctactcagg | aggctgacat | agaagggtca |
| 113881 | cttgagccca | agtgttcaag | ggtgcagtga | gctatgatta | caccgctgca | ctctagctgg |
| 113941 | gacaacagag | caagaacctg | tctttctctc | tctgtctatc | tatctatcta | tctatctatc |
| 114001 | tatctatcta | tctatctatc | atctatctat | ctatctatca | tctatctatc | tatctatcta |
| 114061 | tctatctatc | tatctatatg | caaaagagca | gttaagagac | atgggagata | gttaaagagc |
| 114121 | accaacatat | atctaatgag | agctctagga | gaagagagag | gaggaatggg | caggaaaaag |
| 114181 | gagtgacatc | aatactgtac | ctagatggaa | tgtggattct | gtgtgcctat | aaaaagagat |
| 114241 | aaaaacaaat | ccatatctat | tcacttcata | gtaaacctgc | agatatcaaa | gataaagagt |
| 114301 | acattcaaaa | tcccagaaca | cagaaaagat | agattatcca | gaacagaatg | gctgctaacc |
| 114361 | aaaaagctga | aggatagtaa | tgtgattttt | ttaaaaccag | ccaaagtata | attcaagagt |
| 114421 | aaaatcaaaa | taaagacatt | taaaaacaca | taaaaactaa | gaaccgtagt | aaaacaaata |
| 114481 | atgcactctg | gggaagaata | aatgcaatga | ataagataaa | agcaattata | aaatggaatt |
| 114541 | tgattaaagg | atagtaattt | tttttttttt | tttttttttt | tttaccataa | agtgacttta |
| 114601 | aaaagtacaa | aacagaattt | aaacaagaac | tgtaaaataa | gaagaagggg | tcattcttca |
| 114661 | gtgggttttt | aaaccatact | gactgacacg | tttgtcatgg | tcaagaggag | gatataagta |
| 114721 | ttgaaagata | aaatatgtat | tttttttaaa | aaaaattcag | cgttgtcact | aaaagactac |
| 114781 | aagcacaacc | tacaattatc | aaagccacag | agaaagaaaa | acatacaaac | agaaatgtgg |
| 114841 | aaatatttca | tcagaagaaa | aggcaaaggt | gtaaaaaaag | tcaaaaagga | tggtaaaaat |
| 114901 | tagtccaaat | atgtggttga | tcttaataaa | tgtagctaat | tcaacttatc | tattgagaga |
| 114961 | gaaagagaga | ttattagatt | ggatagattt | gataatagaa | tacaaatcta | ggtatatgtt |
| 115021 | gcttaagaga | ccacctaaag | ctacacagaa | agattaagga | tgaagagaag | taaaaagata |
| 115081 | aaccatgaat | atattcacca | aaagaaactt | cgtttggtaa | tattatcaga | caaattagaa |
| 115141 | tttaagcaga | agcattaata | gaaacaaaaa | aagaacgttg | cataatactt | aaaagagcaa |
| 115201 | aggaatcaga | acaatcacac | acttgtatac | ctctgagaaa | gtagccttga | aagagaaaac |
| 115261 | gcaaagactg | atggaaataa | aactatacac | cttggcattc | aggaagattc | atctgcagcc |
| 115321 | aggcgtggtg | actcatccct | gtaatcccag | cacttgggga | ggccaaggcc | agaattttgc |
| 115381 | ttgagctcag | gagttcaaga | ccagcctgtg | caacacagtg | acaccctgtc | tctaccaaaa |
| 115441 | attcttttaa | attatccaag | cttggtgttg | cacacctgta | gtcccagcta | ctcaggaggc |
| 115501 | tgatgcgaga | ggatcattta | agccaaggag | ttggaggctt | cagtgagcca | tgattgcacc |
| 115561 | actgtactcc | aatctgggtg | atggagtgaa | atcctgactc | taaaaaaaaa | aaaaagaagt |
| 115621 | agtttgcttt | tggcagaatg | aataaaatgg | agctgtgatg | tgtccttgag | caagacagga |
| 115681 | tgtgtaagag | cagaatttag | ggagggttaa | tctgatgacc | taagcaataa | aggcaggagt |
| 115741 | gaggaaacgc | taggtacaga | gattcgcact | ggcaagatcg | gcgtggtgat | ttaagagtcg |
| 115801 | gttaataaga | ccttgatcta | gggtggcaag | agatgaagtg | atcaaagggg | aaggggaatc |
| 115861 | aaaacaacag | acgtgaactg | aataactgaa | tatcaaggat | aaggaagaga | gagtaatccc |
| 115921 | aactggagaa | cggtggtacc | gtgagcagag | aagggaatga | tggctcgaaa | gcaagctggc |
| 115981 | tttcgggttc | atttgacata | cttagttttt | gatttactga | gttttgggta | acatccatga |
| 116041 | gatgactgtt | ctgagatgga | acacaggcag | ctgttggtgg | gaggagaggc | agagagccag |
| 116101 | tctcttggtt | gccatgctgc | cttctcctca | tgtctctggg | gtgtcctcta | atgccctgag |
| 116161 | gaggtagata | ctcttttttt | taaagttcca | gggtacatgt | gtaggatgtg | caggtttgtg |
| 116221 | acataggtaa | atgtgtgcca | tggtggtttg | ctgcacctgt | caacccatca | cctaggtatt |
| 116281 | aagcccctca | cgcgttagct | atttctctta | atactctccc | tccccccacc | ctcccctgac |
| 116341 | aggtcccagt | gtgtgttatt | ccccttcatg | tgttcacatt | gctcagctcc | catttataag |
| 116401 | tgagaacatg | cggtgtttgg | ttttctgttc | ctgcattagt | gtgctgagga | taatggcttc |
| 116461 | cagcttcttc | catgtctctg | caaaggacat | gatctcattt | ctttttatgg | ctgcatagtt |
| 116521 | tccatggtgt | atatgtacta | cattttcttt | atctggtcta | tcattgatgg | gcatttggtt |
| 116581 | tgattccatg | tctttgttat | tgtgaatagt | gatggaggca | gacactcata | ttcaccttca |
| 116641 | cttaaagaac | tggcaatgct | tgggaggcgg | aggcaggcag | atcatctgag | gtcaggagtt |
| 116701 | cgagagcagc | ctggccaaca | tggtgaaacc | ccgtgtctac | taaaaataca | aaaattagct |
| 116761 | ggacgtggtg | gagggtgcct | gtaatcccag | ctacttggga | ggctaaggca | ggagaactgc |
| 116821 | ttaaactcag | gaggcagagg | tttcagtgag | cggagatggc | gccattgcac | tctagcctgg |
| 116881 | gcgataaggg | agaaactccg | cctcaaaaaa | aaaaaaaaaa | aaaaaagaaa | aaagaaaaaa |
| 116941 | aaaataactg | gcaatgcaaa | aatttgtcag | gaaagaaact | aggggaattt | ccttagtaca |
| 117001 | tggccagaag | attccagcat | ctgtgagggt | gaaggcagga | aggtggttag | agcactggcc |
| 117061 | tgcccattct | cctgaagaaa | gatggttctc | agctcactgc | tactctgctc | ccccattcta |
| 117121 | aagtagtatt | tacgggattt | tatttttatt | ttgtcctgaa | tatttttgtc | actcatattt |
| 117181 | actacttttt | agtttctctg | cctattttag | aagtgaatgg | ttctggatgg | atgagaaata |
| 117241 | tcattccctc | caaagtccag | atatgatatt | gaggttttat | acttctgcac | aagtttctgc |
| 117301 | acatctgaga | ggtgcttggg | ctgaacctgg | atggccactc | tctgcaagga | agaggaaaag |
| 117361 | agagggacag | ctcctacaac | aagtgcagac | agtcatggtg | ggttgagtaa | gagaaggttg |
| 117421 | caggagagtc | acagagaggt | tggggcttgt | aaagaatgtc | atgaaagagt | gcagacacca |
| 117481 | acagaatggg | cgttgctgct | attatcagga | taagccagta | aatgccagga | aaaagatgta |
| 117541 | ccagagactc | caggagggta | agtactgctt | gaaggaaaaa | ggagaactaa | caaggatggg |
| 117601 | aaaacatttg | atttcagaag | ttgaagtctc | tggtgtgagt | tgaattaatc | agttaattcc |
| 117661 | tctaaggttt | cagagggaga | aactggctga | tggagggaag | agcagctcag | tgaaggatca |
| 117721 | acgagaagga | agagcgccca | tcacaaggca | gctgaaagct | gattcatcat | taggacgagg |
| 117781 | ttcaagggag | aagtcagtgg | ctgcccacac | tggcgcatcc | gtcatgggaa | ctttctatgt |
| 117841 | caaaaccctg | ccaatgaatc | atgaacattt | tagttgtgat | tcagattcca | cagctgagag |
| 117901 | ggtatagagt | aagctttaga | agtggaaaag | tttctgacat | attctggatg | gtaagaggag |
| 117961 | ggttaacttg | gcagtaatac | tgataaggag | gccaaggaga | aaagctgtta | gcatgttact |
| 118021 | gagtaattgc | attgaccaga | ggtaatccag | ctggatggac | aggaaggagg | ttttgttgta |
| 118081 | aattactgaa | ctgatggggg | cagggggagg | gtgaaattgt | gaaagtgtat | gacaaaggaa |
| 118141 | agctggaata | aatatttttt | ttttgaaatt | atttttaggg | aaggaaggag | aaatagctcc |
| 118201 | agataggttg | cacaactgca | caccaacacc | atgcacattt | agggaagatt | ttatcattaa |
| 118261 | ttgtctcaga | ctcacatgtt | taacatggta | tccataggga | agtggtgttt | gtgactccat |
| 118321 | gtggcaggga | agctgaactt | acttctgttg | gccacggtca | gaagagaaca | ggaatgtgag |
| 118381 | gaaggaagtg | gtgacaggaa | aaacaactca | aagtgatctt | tctgagacat | tacctccctt |
| 118441 | ccttaccatt | ttcaaattct | aaaaagtgca | catccattaa | cattttaaaa | atgcattgac |
| 118501 | atacatgcat | aacttcttga | tagcaaacct | ataactggag | acatttaaat | catgctagat |
| 118561 | atctgataaa | taacaaaaat | acatttttag | ttactcagaa | aatgatagtg | cacccgtttc |
| 118621 | caggaggctg | tacaagtctc | taacaatcca | cccctgcttt | cagacaatct | gtgcaaatgt |
| 118681 | gagtgccaga | gttatctcag | gagctacctc | tcctccagac | agcagaggta | atgtgaacct |
| 118741 | ctgtggtcca | ggcaggctcc | tggaagagct | gggttaccaa | tcctgggata | ccctctgcaa |
| 118801 | tcgccccaca | gaggacagtg | agaagagaac | aggcattaca | tttgcagatc | tttcttgaag |
| 118861 | catgagagat | aggaaagaga | gtcaactgta | tagacacaga | actgactaaa | atggaggtag |
| 118921 | gaggtagttt | aaagtactag | acaaaagctg | aagatgtggc | tggagaaata | tgtgtggggg |
| 118981 | ccagacgtgg | tggctcacgc | ctgaaatccc | agcactttgg | gaggctgagg | caggcggatt |
| 119041 | gcttgaggtc | aggagttcaa | gatcagcctg | gccaacatgg | tgaaaccctg | tctctactaa |
| 119101 | aaatacaaaa | attagccagg | catggtggtg | cactcctgta | atcccagcta | ctcaggaggc |
| 119161 | tgaggcagga | gaattgcttg | aacccaggag | gcagaggttg | caatgagctg | agatcgtgcc |
| 119221 | actacactcc | agcctgggcg | acagagcgag | actccgtctc | aaaaaaaaaa | aaaaaaaaga |
| 119281 | aaaagaaaaa | gaaaaaagac | atatgtgtgg | aaagtatagc | atcagcaaac | tgtataacaa |
| 119341 | caaggaacct | ggctgggaat | aaagtaataa | tgaatgaata | atatctaatg | ttcaaggatg |
| 119401 | acatgaatta | tgttgtacac | ttgggtacct | tactgttgag | catttcagtg | ctgaaaggcc |
| 119461 | ttggagtctt | tcagaacaca | aagaattgca | cttttgatct | gtggcaaaca | catctagatt |
| 119521 | ctattgaaac | attgtctata | tggttttgag | actaggggaa | tgctagaaac | acccttggtt |
| 119581 | caggagtgac | tcagaaatct | agtcccaggc | caagtgcagt | ggctcacacc | tgtaatctca |
| 119641 | gtgctctggg | atgccgaggc | aggtggattg | cttgaggcca | ggagtttgag | gccagcctgt |
| 119701 | gcaacatggc | gagaccctgt | ctttacaaaa | aataaaaaat | tagccaggtg | tagtggcagg |
| 119761 | cacctatagt | cccagctact | caggagactg | aggtgggagg | cttgtttaag | cccaggggtg |
| 119821 | caaggctgca | gtgagctgtg | atcacaccac | tggactccag | cctgggtgac | agaaaaaaca |
| 119881 | aagatacaaa | taaacaaata | accccataaa | aaggaggcaa | aagacatgaa | cagacacttc |
| 119941 | ttaaaagaag | acctacaaac | actaacaaac | atggaaaaat | gctcaacatt | acttaccatc |
| 120001 | agagaaatgc | aaatcaaaac | cacaatgaga | tccgatctca | caccagccag | aatggctatt |
| 120061 | ctgaaagagt | caaaaaacaa | cagatactgg | tgaggctgca | gatccaaggg | aatgcttata |
| 120121 | tatggctggc | aggaatgtaa | attagttcag | ccactgtgga | gagcagtttg | aagatttctc |
| 120181 | aaagacctta | aaacagaact | gctattcaac | ccagcaatcc | cattactggg | tatatatcca |
| 120241 | caagaaaata | aatcatttga | ccaaaaagac | acctgcactc | atatgctcac | tgcagcacta |
| 120301 | ctcaatatca | aagacataga | atcaatctag | gtgcccatca | acagtggatt | agataaagaa |
| 120361 | aatgtggtgc | atatacacca | tggaatacta | cacagtcaga | aaaaagaatg | aaatcatgac |
| 120421 | ctttgcagca | acgttgatgc | agctggaggc | cgttatccca | agtaaattaa | ctgaggaaca |
| 120481 | gaaaaccaaa | cactgcatgt | tctcatgttt | aagtggaagc | taaaaattgg | gtactcgtgg |
| 120541 | acataaagat | ggaaataata | gacactaggg | acttctaaag | aagggagaga | gagaggtaga |
| 120601 | caaaggttga | aaagccaact | attgggtacc | atgctcacta | cctgagtgac | ggaatcattc |
| 120661 | atcttccaaa | tctcagcatc | acaaaatatg | ctcatgcaac | aaacctgcac | atgtattccc |
| 120721 | tgaatctaaa | tagaagttga | aattatattt | aaaaaaatct | agtcccagct | ttttttcttt |
| 120781 | caatgaatca | aagattcatg | aaccacaccg | gatggacaca | aaaccttagt | tgtcaaaagg |
| 120841 | aagacatata | cgggggataa | aagcacacca | taatgctctg | gtatgaaaag | gaggagttga |
| 120901 | ttttagggac | acacgaagtt | ccctgtgttc | gcttggctcc | ttttacagaa | agaaatctgt |
| 120961 | aatggagctc | tacttataat | aacaaattga | taccagggag | atgcccatta | aaagagggga |
| 121021 | tgaagcaaat | ctcgtggaaa | aactagaaga | aaatagcagt | gtaaatgtca | cccaagtagc |
| 121081 | cagtgcattg | aaggcaatgt | tagaagcata | aggaatgttc | cctttctctg | gacatttgtt |
| 121141 | tattttgtac | tttatgggta | atgaaatttc | ttgggcattt | ttatttagtc | acaagccttg |
| 121201 | aaattctgcc | ctactgcaca | cacacacact | aagggagcca | taatacagga | taaaatgtaa |
| 121261 | atgtagtgtc | tttattagac | ttttctgtgc | taaaggtttc | ttttgatttt | ggataaccat |
| 121321 | aaatggggac | ccagaaaact | aggccaattt | tgcgtaagct | cacaggcagg | tgaaggctaa |
| 121381 | tgatgtaaga | ttctaaaata | agacaaagcc | atacagaata | tgagcagtga | aagcaattca |
| 121441 | aggagttgca | gttgtggctt | tcagaataac | tcaggggagg | ccccaaactg | tacaatataa |
| 121501 | aaaagtaggt | gagcaaatgg | cttagtcagc | tcagcagtaa | cagtggcaca | gaattgagtt |
| 121561 | cctgagcaag | tactttggac | aattagcaac | tatcttttgt | ccacatggag | attactgagc |
| 121621 | ttatgataac | catttcaaaa | tgggcacagt | gctggctagt | aagaagcatt | tagtctaata |
| 121681 | acaagagagg | tggcacatat | ataaatgcct | gcagattcct | gtcatgagtg | gagggccaga |
| 121741 | aatggcttac | gtacttgagt | ctgaatcaaa | agaaagtatt | tcacgggaaa | aggcatctca |
| 121801 | ctctcaatct | cccaagccaa | ataaggagat | aggttgctat | tacaatttgt | atataggaga |
| 121861 | aaaaacggat | agtggctagt | tggatggatg | atggatagat | ggataaagga | tggatggatg |
| 121921 | gatgacggat | gaatggatgg | atggtggatg | gatggatgga | tgatggatgg | atgatcgatg |
| 121981 | gatggatgga | tgcatggaca | gatggatgga | tacatacatg | catacttagg | atatataata |
| 122041 | aattgtatat | ttaatagtca | ttagcattct | aatatctatt | gggtgaacgg | aaaatttgta |
| 122101 | gaggaatatg | tacctacatt | ctttgcattc | atcagctaag | ttaattgtca | gcagactctt |
| 122161 | tgggaaaaaa | ttaggagcac | agctaaggtc | agtataggat | ggaatgtcta | aaggggtagg |
| 122221 | ggagatatat | caaccttaat | tgtgtacttt | tggataaccg | aatagggaga | ttcttagaga |
| 122281 | agaagatgga | gggttctcaa | aaatagatga | tgcatgtgta | taagtgttgg | tgtctgtcat |
| 122341 | caattaaaca | aaatccagaa | tgtaccaggt | tgagaatgaa | gaaaatgaga | atgaaaagga |
| 122401 | aatgcaatag | aaaagaaatt | agatcaaata | tagaaggaat | agtagatcaa | aattaggaaa |
| 122461 | aggtattcat | tgtccaaagc | atgtagatac | tagcacttgc | atagcattgg | atcctgttag |
| 122521 | ggttaaagaa | agagaatctc | atgctcccag | agctatctgg | tgaacgtccc | caactgtaca |
| 122581 | tgtggagctc | tgcaggcccc | acgtctgcaa | tagtactgta | gactacatct | ccgtccacag |
| 122641 | ccagcctcgg | catgttccat | ttacaccaca | tggagaaata | aaatgtattt | ctaatcggct |
| 122701 | ttctaataag | tactaattag | tccaggcctt | tgtgatccat | aaagagataa | agagtgagct |
| 122761 | tgcatccttc | catcaaagcg | ctgaagctgc | agctggcaaa | acctcatgct | gcttttcctg |
| 122821 | cctatcttaa | ggaagaaatt | gaaattagct | caatgaaaaa | atctcattta | gattttgcta |
| 122881 | atatgtatta | aaatccaaga | ggtaaaaccg | tgcaagcaac | tgtgacagag | ttcccaggag |
| 122941 | tggtctgagg | gggaccagct | tggggcagca | ttcgaacagt | ctcttccagg | aagtcacatt |
| 123001 | taaaacaaga | gcattaaatc | gtgacggatg | tacagcatca | gccattttga | tgcatctttg |
| 123061 | cattaaagga | cacgataata | cctaagaggt | ggggagaaag | tcaatgcacc | atattgatta |
| 123121 | ttatgaatac | aagagtttta | aagttagaca | gggttttgta | gttataagat | ccaaaatgat |
| 123181 | cttccagctg | aagtattatt | gcaaacacca | ccaagacaat | atccaagacc | aagggagaga |
| 123241 | attcagaaag | caggctgccg | ggcccacagt | ccttgtgctt | caatcaggca | ccaatccaac |
| 123301 | tccatcaaag | ttagacaaat | tatcccaagg | tctccaagca | ccttctccat | taagtgagca |
| 123361 | cagagacact | catggttttc | agtgcatgaa | aactccctgg | gaaacagctg | aggcagattt |
| 123421 | tctcttgttt | ctgcttttac | ctgggtctta | ggaaatgttt | catgttttcc | ccacagcagt |
| 123481 | ttccagaaca | caacatttcc | tcaaggctac | tctagggctg | tcccacttcc | catttcatgt |
| 123541 | ccaaggtcat | aaagtattct | taatgtaatt | gtaaccatga | tgtcttgtgg | ccaggcttgt |
| 123601 | cgtgattcaa | aacaagaagt | gtttagaaat | cgtttgccat | gtagtaacaa | agtctaacgc |
| 123661 | atctttcttt | gttatttgta | acaaaactgt | tccggaaggg | gatgtatagt | gcataggatc |
| 123721 | tgtacaacat | agtgctcaag | ccttaagtta | aaatgcatta | aaatgggctg | ggcacggtgg |
| 123781 | ctctcgcctg | taatcccagc | acttcgggag | gccgaggcgg | gcagatcatg | aggtcaggag |
| 123841 | atcgagacca | tcctggctaa | catggtgaaa | ccccgtctct | actaaaaaat | atatatataa |
| 123901 | aaaattagct | tggtgtggtg | atgggcggct | gtagtcccag | ctactcggga | gcagaggtag |
| 123961 | gagaatggtg | tgaacctggg | aggtggaggt | tgcagtgagc | tgagatcgtc | tcactgcact |
| 124021 | ccagcctggg | cgacaaagcg | ggactccatc | taaaaaaaaa | aaaaaaaaaa | aagcattaaa |
| 124081 | atgaaatgaa | tcttgaagtt | aaagacaccg | tcataggagg | tctgtgagag | ccagttgcta |
| 124141 | atagcaaggt | gggtatggag | gataaagagt | agcagaagat | gagataaaaa | ggtaggttac |
| 124201 | ccagatactg | tgatataaag | cattttatat | aaaaaggagg | gtcacacaaa | tattgtgaaa |
| 124261 | cgctgaattt | tatattgaga | gatgaaaaat | ccatctggaa | atcaaaaaga | gaaaaaaaaa |
| 124321 | aacccacatg | aatggaatac | atctgagggg | gtgaagagta | tggcaggggc | tgagaaaaga |
| 124381 | aaataatttt | gatagtgagc | tttttagatt | cctaagaatg | agaagagata | agcaggagag |
| 124441 | ttaggaggag | gaagctggag | tcctatccag | attcagcagg | aaattatttc | agcctaaact |
| 124501 | agagacacag | tggaagtgag | aaagaaagca | ctcggtttgt | aagatttctg | ttcaggaagt |
| 124561 | ggagtgaggg | tcccgtggga | atgagtgtgg | ccagaggagg | aaatggagga | ataatagatg |
| 124621 | attgcaagcc | cccaggtttg | ggaggcgggg | aggtgttatt | gacagtcagg | aagggaaaga |
| 124681 | aggtgtctta | aaaaggaaac | aaacagggtt | catttttagg | ttggttattt | gatgtatgag |
| 124741 | gagaaacatt | taaaagtcaa | gacttagggg | aggatgtgtc | taggaaatcg | agggcagagg |
| 124801 | ggattgctat | acccatggga | tgactccgtt | tactccacaa | atgtagaaga | aatgtcttca |
| 124861 | gggccaaatt | tcactggagg | tgaaaacatg | aagaacatga | gtacgattac | ctgggagttt |
| 124921 | agcagacggc | tgtatcatcc | agattttgtt | ttcagtgcct | ctacacctgt | gtaccttcca |
| 124981 | gagcattcgg | ttgattcatg | aggcttgtag | aatctgcttt | ttataaatta | aaggaataaa |
| 125041 | tctttggtga | ttatagtaag | gagataatgg | ggaggataag | gagagctcac | agatacaaca |
| 125101 | taggacaaaa | gtcagaattt | tcacatgtca | acacgaatga | aactttacaa | ataactacag |
| 125161 | ggaaggcaga | atagaaggga | gacaagattt | gggcaaggca | aacagagggc | tgattttgaa |
| 125221 | agtacagagg | aagtggtgaa | aacagtgtga | cagaggaaga | aaggatgaga | agaaatagac |
| 125281 | aagcgctgca | aagaaaatgg | aggaggctgg | gagcagtggt | tcacacctat | aatcccagca |
| 125341 | cttagggagg | ccgaggtggg | tggatcactt | gagttcagga | gtttgagacc | agcctggcca |
| 125401 | acatggtgaa | accccgtgtt | tactaaaaat | acaaaaatta | gttgggcatg | gtggcatatg |
| 125461 | cctgtagtcc | cagctactcg | ggaggctaag | gcaggagaat | cgcttgaatc | caggagacta |
| 125521 | aagttgcagt | gagtcaaaat | gacaccgctg | ctctccagcc | tgggtgacag | aatgagactc |
| 125581 | agccccctct | cccacccaat | aaaaagaaag | aaaaagagag | agagagaaag | aaagaagaaa |
| 125641 | gaaagaaaag | aaagaaagaa | agaaaagaaa | gagagagaga | gaaagaaaga | aagagagaga |
| 125701 | gagaaagaaa | gagaaagaaa | gaaagaaaga | aagaaagaaa | gaaagaaaga | aagaaagaaa |
| 125761 | gaaagaaaga | aaaagaaaat | gggcgaaatg | tctagagaat | cagaggtaaa | tgtttggagt |
| 125821 | aaatgacacc | cagatggcca | gggccacctc | tcctttttct | ggagaaacaa | aggcaacccc |
| 125881 | tatcctattc | ttatcagcag | ctaaaggcag | aatcgatgca | agaagattcc | acaggaaggg |
| 125941 | aagttcctga | ttctgatttg | tgtactagga | ctagaggttc | atatgagcag | ggaattgata |
| 126001 | aataaaatga | tcaataatct | agaagtgaac | ataatagata | gcttcaagag | gcacagatgc |
| 126061 | gcaagcctat | tgctgaagcg | gcatcaaaca | tttctgagat | tcaagaaaat | aaccatttgg |
| 126121 | gtctcatggt | gggggcctgg | ggatgctgga | atttaaataa | atgtcccaga | tactgatcca |
| 126181 | gagagaaaag | caagatttga | agatcatcaa | gagaaacaaa | tctgtttacc | tccattaacc |
| 126241 | tccaccccct | tttctccgtc | accctcttct | ccacatatat | acggggaaaa | agactcaagg |
| 126301 | gggtcccagc | aggtggtgat | ttcttgaatg | gtgttcattt | tctacttgca | tgctgtggcc |
| 126361 | ttattttaaa | tttgaattac | tttgtaagca | ggaagaaaaa | tcattatttt | taagtcatta |
| 126421 | agaaagccag | ataactttga | aaattaagcc | aaatgaaaga | tcgagctatc | ttaatgccca |
| 126481 | aatatttcag | cagctattcc | tctcaaaaat | cttttttaat | gttgttccat | acgaggtgtg |
| 126541 | gcatttttgc | caggaggtaa | ggattttcag | ggattatgat | ttcaaaatac | taaaaacaac |
| 126601 | tgctatttat | taaaaaaaaa | atttttttcc | tccatgacag | gtagcatagg | tctgggatga |
| 126661 | agaaagtact | caatagctaa | ttactgggtg | gagtggaatt | ttaaatagag | ttgaattata |
| 126721 | gggatatata | ctctgcagag | agcaaagatc | tggcatctat | tcctagaaaa | tctcatggag |
| 126781 | gctcttctct | ttgggtggga | ggtaacgatt | ccctgaagat | catctcctat | ttgggaaggt |
| 126841 | ctctgtgtgt | gacctctttg | ggcaaatata | ccttctggtt | ttctaagcca | cacttgattt |
| 126901 | caaatatatt | atcaagtctt | cccacaaacc | ataggaatgt | tccagcaatt | tcagcatttc |
| 126961 | tgacttctga | acagtatgac | tcagcctgcc | ctgagaactt | ggaagcgaca | ctcaagccct |
| 127021 | ctgtagatgg | tgtgtgtgaa | gctctggttg | ggaatgaggt | cgctggagat | gcccatagca |
| 127081 | cagcagagct | ctgcgtgggt | catctcaact | ccctcacctg | cttcctctgg | actcagcaca |
| 127141 | ggcttagcta | gaaccctggc | aattggtaac | tggtgttttg | ctcagaggtg | ataaaatact |
| 127201 | cccttttttt | ggcagtgtgc | aggaaagctg | ggactggtgg | ctggtgaccg | gcatgcctgg |
| 127261 | gcccatggct | gactgccttt | gagtgggtaa | agaaatcaga | ggagttggct | caaaaaggca |
| 127321 | gaaatgagga | gtgtcctcct | cttacttaca | tgaataaaat | tcaaaatggg | cagcagtcct |
| 127381 | cttacacgtg | cattctattt | ctttgaccct | gtgtgtgaca | gatcaggttc | cttcctgctc |
| 127441 | cagacaaatg | gggggactgt | tgtccccaag | aaaatgctct | tgaggatgcc | cactccccac |
| 127501 | ccacagatgg | cactggtcgg | ctggaggaag | gggaggctgc | tccggcctca | ctgccatagt |
| 127561 | tacttgtcta | tacctgtatg | ttttctttcc | cttgtgtatg | tgtgtgtttt | cctgttcatt |
| 127621 | tccataaata | aaatgttctt | tctcagctgt | atagctgcct | cccctgatta | attcctgccg |
| 127681 | tggcctcttt | gtaatggggt | ttcttctttt | gctgaaaaca | gcaggcatct | gcttttggag |
| 127741 | ttgagttaat | gggggatgga | agggagttag | ccaagtctcc | gtgggctctc | tgagaatccc |
| 127801 | cacaacctcc | ttacaaatgg | cttccatttt | cggctgattt | ctcctgattt | tgtttatgtc |
| 127861 | tcacaaaggt | agaatatttt | acaggccact | cagaatttca | tgcaacagcc | cacactgggt |
| 127921 | tgaattttaa | gtagtatctt | ccagggaacg | cgggggtgtc | cagttaccat | ttctgccatc |
| 127981 | agagcctggt | tggtcttttg | ccatcagcac | aagaacaaag | tgtcacattt | gctttttttt |
| 128041 | ttttttttct | tcacaagatg | gctaccctga | tctcaccatg | agcgttatta | tctgctggtc |
| 128101 | ttcctcgaat | gcctacgtac | aatagattgc | atctatttaa | gtatccagat | ccttttggag |
| 128161 | acagggcctt | cttctgacac | acacaaaaag | agttttcaga | tggcagatcc | gcaggtagta |
| 128221 | attttaatag | ccactaatgt | gaattaataa | ttctaataac | ctcatttcag | atgtgttatc |
| 128281 | gggacgcagg | agagggttgc | catggaggct | gtattttatt | gctcacagct | gtgcaaactc |
| 128341 | attttggccg | gcagcattcc | catgtctaac | ctctcctttc | attggtttgc | accatgtcag |
| 128401 | gcagtgtgga | tttaacaaga | acggccaaaa | aaacttaagg | aaaagttgtg | atctcaagac |
| 128461 | tctaagcaaa | tgatgctgat | aagcaacaat | aagagagccc | agtgctttag | aagagaattg |
| 128521 | tgcagttata | tattcatgag | attaaatcat | tgctttggca | catttatcaa | tatcaatgtc |
| 128581 | caaattataa | tttgtcagca | attccaaggc | acgttcttta | agtgctttgg | ggcataaaca |
| 128641 | atattcctta | ataaactacc | ttagggatta | ttgcccccta | taagattccc | aaaagtacat |
| 128701 | taatttataa | tctgaggcct | gtaactgaca | acagaccagt | aaacgtttag | ctaagtgtgc |
| 128761 | ctcccacatt | ccctggtcct | acaatagtgt | tttacggatt | tttttttcca | tttaattctc |
| 128821 | acagcattcc | tataaggttg | tcatattggt | ggttttaaaa | cctggacaca | cattttttga |
| 128881 | tactcccttt | gagaggtgga | gactgtattc | tctccggttt | catctgggca | ggtttgtgac |
| 128941 | tgctttgatc | aataaggtac | agcatatcag | ccaagtacag | catatgtgac | actaagtgaa |
| 129001 | caccagggtg | tcttaaaagg | ccatgcatct | tctgtgttgg | tcattgggcc | atttgctcct |
| 129061 | agaaccctga | atcgtcatgg | gagaggctgg | atgccctgag | gctgtcatgg | tgtgagggag |
| 129121 | cctgggcatc | tctagatgct | gcgggtgaca | gcgctagctg | agccagcctt | ccactgaatt |
| 129181 | caacccaacc | caggtcccag | acacgtgaga | ggtaaagcct | gcaggcgatt | ccaacacctt |
| 129241 | gtatatttcg | tctctccaac | tgaggcccca | gacactgtga | agcagagaca | agccatcctc |
| 129301 | tctatggtct | gactgaattc | ctgatcttca | gaatccatga | acataatgaa | atcgttgttg |
| 129361 | tcttatctct | aggttttggt | gtggtttctt | tgttatgcgc | caataggtaa | cgggagcacc |
| 129421 | aaggacgctt | agatgttaaa | cagcttgacc | atgtcccaca | gtaaatagag | gcctgagatt |
| 129481 | taaaaccggg | tctgttagat | gccaaatctc | aaactctttt | taccataaaa | ttataagcac |
| 129541 | cctaagagaa | gtctataaat | agtgttataa | aaacatgaga | aacaagtatt | tttctttctg |
| 129601 | tccatggtca | ttagtgtgga | ctcatcgtgg | gcttgccaat | cctaaacacg | ggaaagctgt |
| 129661 | ggaaaacatg | cggtgatctt | taattaaagt | ctatttttta | aacccacccc | tgtgttccta |
| 129721 | ccaaattgtc | atgtatctga | tttgaaaagt | agccaactca | ttgactttaa | cttgcatctg |
| 129781 | aacccatatg | gcaatctgga | actcaacatg | aaacccccaa | attctgttca | gctctggttg |
| 129841 | ctccatttga | agccaatcat | gggaaatgct | taacagcttc | tcttctttgt | tgccatttaa |
| 129901 | atactgtctt | ctttgttcat | tttttttttc | ttaagttcta | catctttaca | aggtttctaa |
| 129961 | acttttacaa | aacttggcct | tttgggtctc | aggactgtga | aagaaatgca | gacttgaagt |
| 130021 | tagaccatac | taccaacctg | tcagaaaagt | ctctggtgag | atccattcag | aaggttgttt |
| 130081 | gttgttcagg | ccgggcactg | tggctcatgc | ctgtaatccc | agcactttgg | gaggccgagg |
| 130141 | caggtggatc | acctgaggtt | gggagtttga | gaccagcctg | gccaacatgg | caaaacccca |
| 130201 | tctctattaa | aaatacaaaa | attagccagg | tgtggtggtg | ggcgcctgta | atcccaccta |
| 130261 | ctcaggaggc | tgaggcagga | gaatcacttg | aaccccagag | gtggaggttg | cagtgagcca |
| 130321 | agactgcacc | actgcactcc | agcctgggcg | acagagtgag | actccatctc | accaaaaaaa |
| 130381 | aaaaaaaaaa | aaaggagggg | ttgttattca | ataaacccag | acctcatttc | ccttccctca |
| 130441 | tttcctttct | accccacata | gggctggaaa | aaacaaaaaa | caaaaacaaa | caaacaaaca |
| 130501 | aaaaaaacaa | ggtaaggcta | atcttatatt | cagttagagg | tttccaaatt | ttaggaaaac |
| 130561 | aataaaatgg | agataattta | ataaaaacct | ccaacctcat | ttggagctga | Rctctaggca |
| 130621 | acgtatacct | gcactttgaa | aactcttaca | ggcagagtca | agatttttac | ataaactcca |
| 130681 | ttttcctgga | acaaattgac | ttaactttgt | gaagccttcc | ttataatgag | aggtctcagt |
| 130741 | gcaaggtcac | aggtgacagg | actgcttctg | taattagtca | ctaaagcatc | cagaagcctt |
| 130801 | tgggaataag | ggattttcct | tgcgaaggag | gcttatttat | taactgttaa | ttataaSttc |
| 130861 | tcttgtcagc | ttctctgtgg | atttggtgtc | tacaaagaaa | gctgagggga | aaaaattata |
| 130921 | agggcttctg | tgagaagcat | aaatcagaag | ccatttcaca | gtgccctgct | acaagtagcg |
| 130981 | tttgggactc | tcagatggag | taacaggcag | atgcatttgg | tccaccatag | ttgcatagcc |
| 131041 | tcagggctga | gacacccagg | tgactgtcac | agtcctttag | ggctgccact | cacgtttggt |
| 131101 | cctgtggaca | tggcaaacga | gagctttgct | ttgcctgatt | gctgtggtgc | accagcaagc |
| 131161 | ttccagggag | ccccaagttc | tccattcagc | cttgcatccc | atacatttca | tttgatataa |
| 131221 | gttcttatct | cttggactgc | cctgggtagg | gtcactgggg | ttggtgaatc | acgKtggcct |
| 131281 | ttctcctgca | tagtggttcc | tatgacaaca | gcaagtgaca | cttgaaatgt | aagctacctg |
| 131341 | gagagaccca | aagaccacac | aatatcatct | gtgtggatgt | gataagttgg | cttcctttaa |
| 131401 | gtggcaagga | aaggtaccta | aatacttctt | cactgacctt | ttgtggttgt | tagggtatat |
| 131461 | agtttttcag | agaaagaaaa | aatgtcacca | ctattttaag | aaatactgag | catgacatta |
| 131521 | gatatggaca | gcttaatttt | tctacatggc | tgtcttcttc | atggagttcg | tggtataatt |
| 131581 | ctttgcaggt | tatagaagta | cataatcact | tttgacaaat | agagatacgc | atatttcccc |
| 131641 | actagcaata | agtggattag | atgatatttc | agatcaagac | aaccctaatt | aaattactcc |
| 131701 | taactttact | ggcctctgtg | gattgatctc | tctttgacct | tcatagccca | tcagaataaa |
| 131761 | tgaaaagaaa | acattctaat | attttttccc | attaaaactt | tttcatgaac | cttcatatct |
| 131821 | gtatactgca | ttatatacat | atacatcaac | tgaagccaat | aaatgtttgc | ttgttcttta |
| 131881 | tgtctttttt | aaagcctgta | acaatataaa | ttagcattat | gtcaaggaac | tacattatta |
| 131941 | gattactgca | aagaaagatc | atttaaatta | catttttaat | gtttccacat | ttattttatg |
| 132001 | agaaaaaaat | attttttaaa | caaatatcat | ttcttaagac | aagattgtaa | atgtatcctg |
| 132061 | atgtctttat | cttgttaacc | gtaccagatg | aaagcactgt | ccaacataat | gaacaaaaga |
| 132121 | cattttcttt | aaatgggcaa | agtattggaa | attttcttta | caacaccatg | cactacaaat |
| 132181 | tatcttccat | aaagaggaat | cttttttgct | tttcatgagg | tgtctttgtg | gtaaacactg |
| 132241 | taagggggca | tttttgattg | tttgtttttt | gccatttgtg | aaaaggcaaa | gtttatatta |
| 132301 | catattttct | ataaaaagtg | tggatatatt | atgctgtttg | tttaaaaaat | ggtatttcac |
| 132361 | tgcatggcaa | gagtgccctc | ttttaaataa | gtacaaaatt | caaaaattgc | aagtcacaga |
| 132421 | ctactagaac | tcacaaaact | tttttcttct | tttctctttc | tttcttctgc | cccctctttt |
| 132481 | cattacctta | tacaagacag | cttatgattt | tgttaaaagt | gatgactttt | ctagaacatt |
| 132541 | caaaatgtca | tcctgtttgt | gaccgttttc | acagaaactc | tccagggctg | tctctaccat |
| 132601 | gtaatgcaag | gtactcccac | catgggaata | atattttaaa | gttctcctct | ggtagaatat |
| 132661 | tcatacattt | ttagcaacta | gaagcaagta | ggtcttattt | agttttacat | tattcacagt |
| 132721 | ggttggttat | tacaaagcct | tgcacatagg | aggtgctgaa | taaatatttg | aattaacttg |
| 132781 | gatgattgtt | ctgaccaata | ggatgggtga | tttggtgctt | tttgatgata | ggaatagata |
| 132841 | aaacacagtt | caaaaatcat | gcctggcgat | tttgttgttt | tgatgaataa | atctcagtca |
| 132901 | acgttcatct | acctggttaa | tcacatggag | attagccttt | ctgatgcaga | tacacagtca |
| 132961 | tcttgatcat | cacttgatgg | taccagacat | tttgatgggt | ttcctcatca | ccattaggta |
| 133021 | catccagtct | tttctgtctg | ctagaaagtt | ccttctggct | gtatacacac | acacatgcac |
| 133081 | acacacatac | acaccactca | aggtttcctg | agcctgccca | ttttctgttt | taattcttaa |
| 133141 | ttcaaggaca | aaggataaag | gttgattgaa | taaggaaagt | catcttttgc | tttctgattt |
| 133201 | tttttttttt | gatttggcat | tcttacatca | tgctaatctt | gaagaagtat | caaatcctct |
| 133261 | aaaataaatt | gtggcaaaga | aacatcttct | gtctctttgg | ctccttccca | gggaagtctc |
| 133321 | agaagtccta | caattcctca | cagtcacagg | gtggtctctg | ctggaaagga | ttgtattgaa |
| 133381 | catagagtct | ggcaagcgag | aggttaaata | acaattattc | caatgtgggt | aaagtcacag |
| 133441 | tcaacatcat | taaaattgtt | tttttccccc | aatacctttg | tactcattca | tgaataaaag |
| 133501 | tgagttttat | agcagaagta | attactcttt | cttatcctaa | tgaaaatctt | aacaaactaa |
| 133561 | taaatgtgta | accccttcct | tgggtccaaa | tgttgttttt | agttactcta | tacttgtgcc |
| 133621 | tagaagaacc | aaaaccaaaa | taaatgactt | atcttttgtg | ttatatactt | cccccaaaat |
| 133681 | tatatcccaa | agatcattta | aatactcata | acatcacttt | tgaaaacaaa | ttaagttcca |
| 133741 | taacctctta | gcgttcttct | attttcctgt | cttatgatgg | cctgcgcagt | tcaattctcc |
| 133801 | ttttgttttt | tctcttccta | tccagggaac | ctggactgcc | tcctacaacc | tctttctctt |
| 133861 | tcccctccaa | aaagcaattg | aaaacatgtt | tgtttgctgc | ccagagggaa | ttcatccatt |
| 133921 | taacataaga | ttgcacagga | attccccaaa | tagagaacat | tcaacatttc | ctatgatgtc |
| 133981 | aacaggaggc | caaactccag | aaagagcagc | cggttcttgc | ccgatgccca | tccttgggtg |
| 134041 | ggttgtgtct | gagtgctgtg | caagctccca | tctggcacat | ttcttctcac | tgagatattt |
| 134101 | ccactgttca | ttctctcatc | cggcctattc | tcctattcac | ttgaataatc | aatagcagca |
| 134161 | acagcctaat | tacccacatc | atcctcgctg | tgctgaaatc | atgagtacga | agggtgcaat |
| 134221 | tgcttcctta | ggaattcatt | gccttcttaa | caagaaccaa | cgcaaactgt | cttacgactg |
| 134281 | gcatctctcg | agcagaggct | gggagctgat | gaagtagccc | tggtctcacc | tcagtagcaa |
| 134341 | atattacctg | cccttctcca | cctcccttca | aactgccRac | aacaccaggg | ctggcttcct |
| 134401 | taaaatgtac | ctttgggatg | cccacgccat | atggcagctc | tgagaatgtc | accctatgct |
| 134461 | ctagggtatt | tttccccgta | gcttacacaa | attgtaaatt | attatccata | aaataatggt |
| 134521 | aaagagagac | aagggcaagg | aaaRgatctg | ccgtaaagcc | tccattagca | aatgagattg |
| 134581 | gctttcctat | tcatgtgaaa | agttcaagga | ggcaatcttg | ttaaacagcc | attttgcata |
| 134641 | aagaaataat | atgccattat | ggaccttttc | aaataacttt | gatgttttcc | agaacaattt |
| 134701 | attaatactg | tactttggag | aaataagaga | tgaagtctca | ctagagaata | aaatcggtgg |
| 134761 | ttgttgggca | gttggtcata | gctcagcagg | tttcatagct | gtctgaaaca | catttccgtc |
| 134821 | ctctgctaga | agtgcttgat | ggcaaaattt | ccccccaact | caactcccct | tccccgtttt |
| 134881 | atcttgggat | tatttgaatc | agagaaacag | tcctgatctt | cacttctcta | tacaataagc |
| 134941 | aacagtagta | taattgcaat | ctgacttctt | tcttttttcc | tctccctccg | tcaataccca |
| 135001 | ctgaccttcc | taagactagg | ggggaaggag | agtgataggg | ttatgaccta | ggtttataag |
| 135061 | aggcatgtta | atgatttacc | tatttgaagt | aagttacatg | aacaagactc | ataatgtagc |
| 135121 | tccttggctg | gattcgtacc | tgcctagatt | cacctgtgtg | ttgaactcac | acaaaccact |
| 135181 | tcatactaga | tccttgcctt | tatggaattt | gcacagtagt | gcctgcttct | aagtgtccca |
| 135241 | ctcagtcttc | tgttctgttc | tcctaggtag | cttacatgaa | agaagacggc | atccaggcac |
| 135301 | cttcagcttt | tctgtgtttt | gggtactttc | caagaagtga | ttacttatca | tcgtgttatt |
| 135361 | aggttggtgc | aaaagtaatt | gcttttttgc | cattgaaagt | aatggcaaaa | ccgtgattac |
| 135421 | ttttgcacca | acctaataat | tactgtattt | atgtgagtgc | attctgaaga | atactagttt |
| 135481 | tgcaaggtgt | cagagggatc | cgttgccaag | cacatttgaa | aaaccctgga | aaagaaagtt |
| 135541 | aaacaggtac | tttggccatg | gaaatgtaat | tacaaagctt | ttaatacatg | cattcatttt |
| 135601 | gtgcaacttc | tccaagaata | tataatttgt | tgcatttcgc | aaacttactt | gacttttgtc |
| 135661 | aaacatgctt | tctgcattaa | aattacagaa | ttacggaatt | gtgaattaca | gagggagaag |
| 135721 | aattggtttt | cctcagcctc | aaatatcata | tatgatccaa | ggtgttttga | gaaccagtca |
| 135781 | ataggatggg | aatggtatgg | tataacctct | agattctaga | gatcctctct | catagtggtt |
| 135841 | tcgttgaggc | aagttgccat | attggagagg | cccatgttgc | aaggtgtggg | gaacgcctac |
| 135901 | ctacctgcca | agccaaaagc | cctccagaga | ccgaatcctg | ctaacaacta | catagtgatc |
| 135961 | cttccctaRt | tgaggcttcc | gatgaaactg | caagccctgg | gctagcacct | gaattgcagc |
| 136021 | ctgtgagaga | caatgaagca | gagaatcagt | gaggccatgc | ctagatgcct | gacccacaga |
| 136081 | aactgtgaga | tcataagaat | tttaagtcac | aaagttttgg | aggtaatttg | ttatatggct |
| 136141 | atagctaacc | aatacagatt | ttggtttcga | gtctagaata | atgtgaatca | ttgtctcagt |
| 136201 | aggattaatt | tggaaaatta | taattgaagg | tataagtcct | gggcagagtt | ttgaggaaga |
| 136261 | aaaacacatt | actcttcttg | ggataagaga | caatattcca | gttatgaata | aaagaatgta |
| 136321 | caagggctta | taatgtcaat | cattgtggag | aataattcct | gagaatttat | taaatgcatg |
| 136381 | gccctatggg | gaatagaaaa | atgaatcatt | ttttcttgtc | ctcaaggaac | tttctcccta |
| 136441 | gacaagaagg | aaaataaata | attgagcatc | tattgttaaa | agtagaagta | aaacaaagat |
| 136501 | acaaagagct | atcgaagcca | tactcccata | tatgcaaatg | agtgagatta | gtccczaacag |
| 136561 | agtggaagta | gagaattaag | aaagtttcct | tggaggaaat | gccaatcgaa | ttgtccttga |
| 136621 | ggagtagaaa | ataaaatcat | agtattttaa | caacaggtgg | aaagggtgaa | caaagtcatc |
| 136681 | tcctggggaa | aggagaacat | aagaaaaggt | tcagatataa | ggaggtatgg | ggtaaattcg |
| 136741 | gagYaagtac | agcaagtatg | tgtagggcat | cgaatagagc | atctttcaga | agaaaacaga |
| 136801 | aaccaaaatt | gaatagccaa | atgattttgt | tttatactgg | gtgggttttt | atttcaacaa |
| 136861 | gttatgttaa | gtgaatcaaa | atttggctac | cagaatgaaa | aaattagaat | ctcaatttcc |
| 136921 | atcattccgg | tttcagctct | aagcctgaca | caggcaatga | acacctcttt | taggtaaacc |
| 136981 | agaccactct | ttttgcaaat | ggcctcaaga | ggtccctgag | tttRaSccca | tgacttgaga |
| 137041 | taacatgaga | ttgaaacctt | ccaagcaaaa | gaccatcaaa | cccttagtca | cctctagata |
| 137101 | agattccaca | cgatcccttg | gaaacttctt | ctaatgtttg | acaactttaa | cagttagaca |
| 137161 | gcttgtcttg | gttttgtatt | tcttaatgct | taattaccca | cttttaaact | gtgaagataa |
| 137221 | atgtaagtct | tctctggaaa | gttctgagtt | tctctgactt | ttcttcagag | gtcctttggc |
| 137281 | taagtcatta | ataattgtca | tgttctcctc | tggacctagt | ccaaattttc | tatgtctctt |
| 137341 | tagtagtaaa | gaccccatct | tggcagagca | cattaacaaa | gggcctaaca | tatgacaggt |
| 137401 | agggttaaaa | aatgacttgg | ctttcacatt | ttatattccg | cacacgtgac | atcatgtgat |
| 137461 | gtttacctcc | tagtgtcagt | accgtgtcat | gggcccacat | ttgtcctgtc | tatcatgact |
| 137521 | tccagggcta | cagataagtc | ttggctctag | gaacagctca | ctaaacttct | ctttgagagc |
| 137581 | ctaaaattta | ggagatatta | cactacacag | gggttgtatt | ggaacaaata | cccgatatga |
| 137641 | ttagaatttt | aatgttcgaa | cactgaaaaa | gaagcagcct | tggccgggcg | cagtggctca |
| 137701 | cgcctgtaat | cccagcactt | tgggaggctg | aggtgggtgg | atcacaaggt | caggagacca |
| 137761 | agaccatcct | ggccaatacg | gtgaaacccc | atctctacta | aaaatacaaa | aaaattagat |
| 137821 | aacatgagat | tgaaaccatc | caagcaagtg | ccagctactc | gggaggctga | gacaggaaaa |
| 137881 | ttgcttgaac | ctgggaggcg | gagattgcag | tgagccgaga | tcgcaccact | gcactccagc |
| 137941 | ctggtgacag | agcaaggctc | tgtctcaaaa | aataaaaaaa | gaagcagcct | taaagctgcc |
| 138001 | ttctcttggt | aagttagtat | gaacttacct | aagaaccttc | atagacatta | tctttttgtt |
| 138061 | taagagacag | ggtcttgctc | tgttgctcag | gctggagtgc | ccccaactct | tgggctcagg |
| 138121 | taattctgcc | tcagcctcct | gagtatctgg | gactacatgt | acatgccacc | atgcctggtt |
| 138181 | aatttttttt | ttttaattta | ttttttagag | atcgggtctt | gctatgttgc | ctagggtggt |
| 138241 | ctaaaaatcc | tggactcaag | ctcttcacct | gcctctgcct | cccaagtagc | tgggattgca |
| 138301 | ggcatgagcc | tggccgtcgg | cattatcttt | gacagcctca | caaatatcct | gccaaggatg |
| 138361 | ttaagtggtt | cctaattcta | tcattgtttt | accattgagc | aaactaatgt | tcagatatgg |
| 138421 | tcacttatct | acataagcaa | attttaagtg | gtgaacttct | acaaatcgaa | tctaaactaa |
| 138481 | agaaaccctt | gttggacctg | ttttgcttcc | agattctctc | ctaacttatt | aaggtatata |
| 138541 | agccccaaat | cactaataag | aaaatgcaat | ttaattgtaa | tgtagtaggt | gaaatgtagg |
| 138601 | cacaactgct | atttttgaat | aaggatatga | atgagaggat | caatgaagtg | tgtgtgtgtg |
| 138661 | tgtgtgtgtg | catgtgtgtg | tatgcatgta | cagcacagcc | aacaagtggc | tggacaagac |
| 138721 | actactcagc | tggcaccatg | caaaaagaca | cctgtaaaat | ttctgtctgt | ccccttttca |
| 138781 | gacaaattca | gtctctctgt | cctgtcccct | taatacctaa | aagtgtccat | cattcttggt |
| 138841 | accaagtgaa | gagctctgta | agccttcctc | tcccttgaac | caggggacct | ttcactctcc |
| 138901 | ccaaatggtc | cagtatttgg | cagtaaatgc | caacacatct | tgctgtaaat | atctcggtgt |
| 138961 | gtgtgtgtgt | gtgtgtgtgt | gtgtgttgtg | tgttgtgtgt | gtattcctag | gtctgggttc |
| 139021 | ttggtggaag | tgcccacctt | gccagaagaa | actgttctta | actccacctt | tagaaattaa |
| 139081 | acatttgttt | ttaaacaaac | gaaaaagaaa | aaaaaaccca | aggtgtacaa | ctgtttaatg |
| 139141 | gctaaggaag | tcctagagta | cacatacctt | aataccagtt | tctttctatg | aaaccataga |
| 139201 | agtgatagtg | aaaattaact | gggaactctg | gtgcttggga | atcacatcat | tatatgatta |
| 139261 | tactgttttg | tcattataaa | attatggacg | tcattgtgta | gtaggaatcg | ggaattgtac |
| 139321 | aaagctagac | agcaaattat | attctaagta | ttccctttgg | aatcaattaa | agaacatctt |
| 139381 | taacaaatta | ggccctttgt | ttcatcatta | gaaaggaata | tataaacagg | atatttaggc |
| 139441 | aatgaaatat | tttccattca | aattaattat | agcaaattag | ctagagaatc | tataataaat |
| 139501 | tgcaaagttt | gacaccttat | gaacttatta | aagtgaaagc | tttcctctta | gcctttaaat |
| 139561 | tattaatttt | catggcctaa | aattataatt | taatagtttt | attggcatgt | gataattttt |
| 139621 | aaattacaag | actatcgtca | tcccactagg | tctttctatg | cataactgaa | aattacttac |
| 139681 | agcattttag | tagcaatgga | aaataatgaa | tgaatgctca | aatctgattg | gttacataag |
| 139741 | tcatttttca | gctcttttgc | taaacacaga | taaaaactct | tttctcaata | aaccacaaat |
| 139801 | ttgtacctaa | aaaagaaaaa | ctggctattt | tattactttt | gcctattttt | gtgaaaattt |
| 139861 | cacgttgtat | tgattttttt | cttttctcta | ttctttccct | tgagtcaaac | tcttgatttc |
| 139921 | tgccgtgaca | acaggaaaaa | agaaagtgca | ttcagcattt | ctgtccaatt | cctccctttc |
| 139981 | cctttacagc | aacactcctc | gaaagaattt | tctataacag | atctctatgt | cttacatgat |
| 140041 | tctagcactc | agtcagccct | cgccagtcag | gtttctggcc | tcatcatcat | cctctgaaac |
| 140101 | tgctcttttc | aaagtcacta | atggccattt | tttttttcct | tctcttactt | gacttctaag |
| 140161 | tatctttcac | aacacttgaa | tggttcctgt | ttcatgaaac | gccgtctgct | cttgacacca |
| 140221 | ctctcttcct | cctactttca | tggctacttc | ttcctagtct | cctttgttct | ctccttttcc |
| 140281 | tctttcaata | tccaaatcat | aaccccagct | ttcttctctt | ctataccttc | atttcttccc |
| 140341 | tagataatct | taacaagtct | cattgctgta | agtatcatct | atatgataat | gaaacctgaa |
| 140401 | tctattacta | ctgtcttgac | ctccactggt | tgtctgactt | ctacatttaa | atgactccta |
| 140461 | ggctgtcacc | ctggtgttca | aaacagaatt | atctacttct | tcctaaaaac | gtattcccag |
| 140521 | tctgcctctc | accagcacac | tattccagta | tccccccagt | tgctcaggcc | aagaagctag |
| 140581 | atgtatttct | tgaatcattt | tattcccctc | acttcctatt | ttcaatYcgc | gccaagtttt |
| 140641 | gtcaactcta | cttttaggac | acatcccaaa | cttgcctgct | tttgtctatc | atctgctact |
| 140701 | cccacccttg | tctctaagtc | gtcatcatct | cggctgcaac | gttgctagag | ccttcttagt |
| 140761 | ggtctctctg | attctaccct | tgtcaccctc | caccctgcaa | ccaccctgag | aaacccttct |
| 140821 | ccacacagta | gccagagtaa | cttactgaat | cagacgttct | cactcccatt | tctaaaattc |
| 140881 | tccgttaaat | tccctcgcat | tgaaaatcaa | ctccaaagcc | tataccttgg | cttttgaaga |
| 140941 | tcacttgatc | tggcctgttt | gcctttccga | gctggccttg | cacctttctg | ttctactctg |
| 141001 | tataacaaca | ttctatgtgg | caggggctcc | actggccata | cacatgagtg | aagggcggag |
| 141061 | cagagcccca | aaggggcccc | aggataagcg | tatgttaagc | tatgaagatt | tttgaggttg |
| 141121 | gctgtttcta | cggcataacc | tagcttgttt | ttgctggtac | agggaatcat | ttacatattc |
| 141181 | gttcacaaaa | tacatatttc | ttgaatgtgt | agcatatgtc | agacattact | tttggaataa |
| 141241 | tacctctaac | tggggataga | caaatttcac | tctctcatgg | ggtttacttt | ctaatgaggg |
| 141301 | gtgcatacga | taaacaagaa | aacaaataca | tgaataagat | acttttagat | aatgacaatg |
| 141361 | agcagggtgt | gccccagcct | gtcttcaaac | tcagcctgag | gattataaac | ccagaaYacc |
| 141421 | atggctRaca | ccacaaactt | aggcagatga | cagtcccaag | gtaataaaaa | cagccacaaa |
| 141481 | atgcttccaa | gacaagtatg | aagttcaagt | tctctgcaag | aaaggtgatg | aatgaaatga |
| 141541 | gcaacattat | taacaatact | tccaaaagca | gataacgtaa | aaacaaaatc | ttaaaaaaat |
| 141601 | gactaataat | tctcggattt | aaaaaattat | gcaagaactc | acattaaaag | atcatacaga |
| 141661 | gtgttatgta | agacagacaa | aagaaaaata | tgtacttaaa | acacaatgta | gaataattta |
| 141721 | agaaaatgca | aatcaaagag | actatttaag | atttcaaaat | gaaaagagag | tttgcctaga |
| 141781 | attgagtatc | acttttttta | gcgaacattt | caccttctat | tttttatctt | tttcaggtta |
| 141841 | acaacttttt | aaaaaattaa | aatccaatat | ttagactaaa | gttattatta | atttagttgc |
| 141901 | atcctcagat | ttattgggaa | ccttggagta | tcttttattt | tttctaagtt | tttaaaagta |
| 141961 | atgtacaaca | tccatatata | ggattgttca | atgaacaatt | atacaaatga | acaatgtata |
| 142021 | tatggatgct | atacattaat | ttaatgaaca | atatgttcat | aaatgttgag | ctcaatatat |
| 142081 | ttttacaaag | aaaacaacct | tgaaacaatc | accaagttca | agaaagaatg | tatgtagagc |
| 142141 | agtcctcctg | cccccaatcc | ttcccccaag | gaaccacaat | tctaagttct | aagcataatc |
| 142201 | aggttttgcc | tttaggtcta | tttttgaaYt | ttacaaatac | tttcatattt | gtctttcctt |
| 142261 | ttgcttaaca | ttatgtttgt | gactgttgtt | tttattattc | cttccttatt | gtgctgggta |
| 142321 | gtgttctatc | agttgaaatg | tcccatagtc | acattgttcc | gttctgtggt | tgatgttctt |
| 142381 | tgtgcaagtt | tccagctcag | agctattgta | agtaattctg | acacattcat | gtttgtgtct |
| 142441 | tttgttgcac | atctgtactc | atttctgtag | attgtaaata | agtagtgatt | gtcattgtac |
| 142501 | agagttcYgc | atcatctgtc | ttttaacgta | accattatcg | agtatgtgga | gtgttattgt |
| 142561 | aatattaaat | tatggttcta | atttgcatta | ttctattaac | taaacagatt | gagcccattt |
| 142621 | tcataagttt | gtttaccatt | gggtatcatc | ttttctaggt | tgcttgttca | ggtctcttgc |
| 142681 | ccatttcccc | ccatagtctg | tctgtatttt | tctaatgcaa | gcatcatatt | gatatcagac |
| 142741 | tttcaacagc | cacatttaac | aaagagaaaa | atggaataat | agtttcacag | tgcaaaggaa |
| 142801 | aataattcaa | acctagaata | ctttttaaaa | aaaatttagc | tgagctttta | tttaaatgca |
| 142861 | aatgtgaggt | aagacaattt | ttagatattc | tacaactcta | aatgtttatc | actcaaagac |
| 142921 | tcatttggaa | aggactttgg | agaaatgttt | aaatgtttct | ccttgattcg | tagagaatca |
| 142981 | aatcaaggag | aaagcaatga | gatattgcca | actaggcaag | tacaaatttt | caagtttatt |
| 143041 | atcaattaag | taaataagga | atatgaagaa | aaatgtagat | tctgtaaata | agaaagcaaa |
| 143101 | gcattaagca | tactcacttg | actggcaagg | gttgggggtg | ggggtggagt | cctagtctta |
| 143161 | acaaaatata | cacataatat | ttgtggagaa | aagttttaaa | ctctactgaa | ggatattaaa |
| 143221 | ttatagctaa | aacaaatgaa | gaagcagaac | atgtttgaag | atgggaagac | aacattttaa |
| 143281 | aaatgttaat | tttcctcaaa | attaatgtac | atatttaata | ttactcccat | cggtatccca |
| 143341 | gtctgagttt | tggtagaatt | agaccaacta | tttaaaattt | gcgtagacac | aaaaatgccc |
| 143401 | acaaaatatt | cttaatagga | agatctttgt | agggatctca | gtccatcatg | tattaagaca |
| 143461 | tatttcaaac | ccataatatt | aaagtaattt | agtgctggga | agtgatcagt | tagagcaatg |
| 143521 | gatcagaaac | agatccaccc | atttggaaac | ccaatatgtg | acagtagaga | tgctacaaat |
| 143581 | cagagggaaa | tatccggttt | ttagtaaact | gtgttagaga | attatttcac | catatgtaca |
| 143641 | aaaatggctc | atgggcagag | aatggggttt | aagggattgg | tgagtaaaac | gtgacaaata |
| 143701 | ggccacatgg | aattataagg | ggccttaatg | accatgggga | agactttgga | atctgtttta |
| 143761 | tttacttatt | tatgtttgag | acagggtctc | actctgtcac | ccaagctaga | gtacagtgtt |
| 143821 | gccatgatag | ctcactgcaa | ccttgaactc | ccgttgctca | agtgatcctc | ccgccttagc |
| 143881 | ctcccaagta | gtagctggga | ctacagacac | atgccaccat | acccacctaa | ctttaaaatc |
| 143941 | tttcttttac | tttctttctc | tttctttctt | tctttctttc | tttttctttt | cttttctttc |
| 144001 | tttctttctt | tctttctttc | tttctttctt | tctttctttc | tttccttctt | tctttctttt |
| 144061 | cttctctctt | ccttccttcc | tttcctttct | ttttctttct | ttctttctct | tttttttttt |
| 144121 | tttgagacag | ggtctcactc | ttttgctcag | gttggagtgc | agtggcatga | tcttggctca |
| 144181 | ctgcaacctc | tgtctcctgg | gcttaagtga | tcctccctcc | tcagccccca | agtagctggg |
| 144241 | actacaggtg | cgcaccacta | tgactggcta | atttttgcat | ttttttgtag | agactttctc |
| 144301 | actatgtcac | ccaggctaga | cttgaactcc | tgggcttcag | cattcctccc | actttggctt |
| 144361 | cccaaaatgt | tgggattaca | ggcatgagcc | actgtgccca | gcctggaatc | tgttttaaat |
| 144421 | aaaatagaag | tcataggaga | actttatgta | gaggattaca | tgatttgatt | tacatctcca |
| 144481 | aaagatcacc | agtttttgag | cagagaatgc | attctagggg | caagaatgga | atcagagact |
| 144541 | gaaacagatg | atattaaggc | aggcaaatcg | ttagagtcag | acttgaccac | agtaacactt |
| 144601 | aatgtaagaa | gagagtggag | caatggtaaa | caaaagatct | gaagaaaagg | aaacgtgaag |
| 144661 | taagaaagta | aagaatgtta | tacccaggga | agttgtccaa | gtataggggc | aagagctaca |
| 144721 | catcttcaag | aaggtgagga | tttaaggagc | acctctcccc | taaggtcttc | ttcaacaacc |
| 144781 | tgtggaagag | gaaatctagc | cgagagaggt | gctaaacaga | aagttaggaa | aaaggagaaa |
| 144841 | ttgtggtaaa | aacacataaa | caaaaacaaa | accaaaaacc | aaaataaccc | gaggtattag |
| 144901 | cacgatgttc | ataaatacag | aatgacatct | gggctactat | gagaataata | gacaaagacc |
| 144961 | agaatgtgaa | tgttacaata | gtatgtcact | gaggaggaaa | aggagagagg | aggagaataa |
| 145021 | tggtgcgaat | ttctgcattt | ttaaagtagg | aaattggacc | gggcatggtg | gctcatgcct |
| 145081 | gtaatcccag | cactttggga | ggccgaggtg | ggcggatcaa | gaggtcaaga | gattgagaac |
| 145141 | atcctggcca | acatggtgaa | actccgtccc | tactaaaaac | acaaaaaaat | tagcggggcg |
| 145201 | tgatgacgtg | aacctgtagt | cccagcgtct | ggggaggctg | aggcaggaga | atcacttgaa |
| 145261 | ctcaggaggc | ggaggttgca | gtgagccaag | atcgtgtcac | tgcactccag | cctggcaaca |
| 145321 | cagctagact | ccatctcaaa | aaaacaaaaa | caaaaacaaa | aaaagtagga | aataaaaaaa |
| 145381 | tgtggctgga | cacagtggct | catgcctgta | atcccagcac | tttggaaRgc | caagacaggc |
| 145441 | ggatcaactg | aggtcaggag | ttcaagacca | gactggccaa | cctggtgaaa | ccccatctcc |
| 145501 | actaaaaata | caaaaattag | ccgggcgtgg | tggcacatgt | ctgtaatccc | agctacttgg |
| 145561 | gaggctgagg | caggagaatc | acttgaaccc | gggaggcgga | ggttgcagtg | agccaagatc |
| 145621 | tccccattgc | actccagcct | gggcaacaaa | atggaaactc | agtctcaaaa | aaaaaaaaaa |
| 145681 | agaaaagaaa | aaaaagaaaa | aatgggaccc | taacttttaa | atcctttttt | aaaagtatca |
| 145741 | ttttatttca | atattacttt | atgtgaaaca | tttaaggact | aatttctctg | ttgctgaagg |
| 145801 | aatatctgaa | attcaccaaa | ttcattcttt | cagttttatt | ttatttcatt | ttcccctact |
| 145861 | gaattttaat | taaccttaat | agttgtatat | ataattaccc | cttttctttt | agcatgtata |
| 145921 | tgaatgtgtg | tatatgtata | cacacagaca | gatatatgtg | tatatctttc | tatgtgttga |
| 145981 | tcatccatct | atttctacat | atgcatgtct | agaatggcat | gaagaaggtc | agttctgggt |
| 146041 | agtagactga | ggattttctt | ttcctcactc | ttctttctta | tctataagtt | ttaaataaca |
| 146101 | atcatgctcc | acatctacaa | aaacaagtga | tgtcattatt | ctaaagacta | gagtgaaaat |
| 146161 | aggtgcagag | ggaggaggta | agcaaaatgt | attttgcata | attttctaag | aaaaatgaga |
| 146221 | tattacatgt | cagtttttag | aacagtgtct | ttcacttagt | aggtgctcag | tgaaagatgg |
| 146281 | caatgatgat | caggattatt | atttcccaga | cttccaggga | gtctctcctg | cctccttcaa |
| 146341 | tttctctgcc | attgactcta | aaaaactcct | tggacaaaat | gcttttgtcc | atttaccaat |
| 146401 | agctctgtgt | tgctcaggaa | tggaggggaa | ggaacagaaa | tgagcagaca | tctttgttct |
| 146461 | atcaattttt | gggagaccag | ggaaacaaaa | taaaacaaaa | gacaagaagt | gcttcatttc |
| 146521 | taagcatcac | aatggctgaa | attatgcaac | ttgcatgtag | tctttagaga | ttccagttaa |
| 146581 | gccttcaaaa | ctaattatcc | agcttgttgt | ggtggtggtt | gtttgtttgt | ttttgcaatc |
| 146641 | cctgtattag | tctgctcggg | ctgccataac | aaaacgcctt | agcctgggtg | gcttaaacaa |
| 146701 | caggaacttc | ttttcttaca | gttctggggg | ctggccgaga | tcagggtgcc | agcttggttg |
| 146761 | atttctggcg | agggctctct | tcctggctgg | tgactgctgc | tttctcattg | tgtcctcatg |
| 146821 | tgacctttct | gtgatgcacg | ctcaggattg | aggaagagct | ctggtctgcc | ccctttttct |
| 146881 | taaaagaaca | ctaatcctgt | gggatcaggg | ccctaccctg | atgacctcat | ttgactttat |
| 146941 | ttacttcttt | aagggctcta | tacactcata | ttggggatta | gggctttttg | acattaattt |
| 147001 | tggagggacg | taatttagtc | catagcagct | tctcaatgtt | tttacaatgt | atttgtttgt |
| 147061 | tttgttttaa | aactaaaatc | catagatggt | ggcatttctt | ttttttaaga | gacagggtct |
| 147121 | gctctgtcac | ccaggctgga | gtgcaggggt | gtgattatat | ctcagtgcag | cctcaactcc |
| 147181 | tgggctcaag | ggatcttcct | acatcagcct | tcagggtagc | taggactaca | ggtgtgcacc |
| 147241 | accatgccag | gctaattttt | aaaatttttt | tgtagagaca | gggtctcact | aaattgccca |
| 147301 | gactggtctc | aaactcctgg | gctcaagcaa | tcttcctttg | gtgtgccttg | gcccaccaaa |
| 147361 | ctgatgggat | tataggcatg | agatactgca | cctggcccta | taattcttat | ttttattttt |
| 147421 | tgagatggag | tttagctctt | gtcacccagg | ctagagtgca | atggcacaat | gtcaactcac |
| 147481 | tgcacccttt | gcctcccggg | ttgaagtgag | tctcttatct | cagcctcctg | agtagctagc |
| 147541 | attacaggtg | cacgccatca | tgcccagcta | atttttgcat | ttttagtaga | gacagagttt |
| 147601 | caccatgttg | gccaggctgg | tctcgaattc | ctgacctcag | gtaatctgcc | caccttggcc |
| 147661 | tccctaagtg | ctgggattac | aggtgtgagc | cactgtgccc | gactggccct | aaaagttttt |
| 147721 | aaatgctcta | ttgtcccttg | aacataagag | taagacttga | atctatataa | ttgatagaga |
| 147781 | ccttaaaaca | tttctacctt | tcttatccaa | ttttttccct | ctccatctat | accatcctac |
| 147841 | agagacggat | tcctatacta | aagaagggga | ttccagccac | tcacttctgg | gcggccagta |
| 147901 | ttggcattta | agccactaaa | atattttttg | atatctaatt | gaatttgtct | aatttgaatt |
| 147961 | acagatacca | ataacaaaat | cattagttta | ttcttttagt | atctcaccat | atgcaaagca |
| 148021 | tttttgtgtg | catttggttt | gtttgtttta | tcatttcaac | catcttaaga | agtagagaga |
| 148081 | gaagatctag | tcctaatttt | atacaggagc | aagtagtcaa | gtgatttacc | agcaacaata |
| 148141 | acaaaaaaca | acaagcaagg | gacagagcca | gcaagaaagt | tcattttaaa | gaacaattgg |
| 148201 | catagccggg | tgcggtggct | cacacctgta | attccagtac | tttgggaggc | tgaggggggc |
| 148261 | agatcaactg | agatcaggag | tttgagacca | gtgtggccaa | catggtgaaa | ccccatctct |
| 148321 | actaaaaata | caaaaattag | ctgggcgtgg | tgggggatgc | ctgcagtccc | agctacttgg |
| 148381 | gaggctgaag | cagcagaatt | gcttgaaccc | cggggagatg | gaggctgcag | tgagccaaga |
| 148441 | tcgtgccatt | gaactccagc | ctgggtgaca | gagcaactct | ctgtctcaaa | aaaacaactg |
| 148501 | gctgtttttt | tcccctctct | tttctgtttt | tactgtgcac | tgtttcaaca | gttaaataac |
| 148561 | tttaaaaata | attaattcta | taagaaagca | tgagaagggt | actgtaagag | aataaaaaga |
| 148621 | caaatgagac | atgttttttc | tcccctaagg | agcttccatc | agggtgcaca | gtgtttcatg |
| 148681 | caatccaaac | agagcattgc | tgcatgaaga | gggttatgtc | tttccattcc | tctttaaatc |
| 148741 | aactttatct | gtgagaatct | cataattttg | aattatggtt | tagtgttcat | ttatggttag |
| 148801 | gattatgatt | aaagtgcaat | taatctctga | taaagccatg | tctacattga | ttcactaaat |
| 148861 | cagaattttt | gagggtatat | tttgctttca | tgattccttt | aataaccaac | ttctggctaa |
| 148921 | tcagacaagt | actgcaataa | atccatgtgc | gtcattgatg | ataatatttg | gattgacttt |
| 148981 | tctttataat | attttgagtc | atggcataat | atcgatattt | atacctctKc | cagtcctaat |
| 149041 | gcataccact | ttgattaata | aaatactttt | aaaaatactt | aaaatatagg | taataacaaa |
| 149101 | ccaacaatca | atctttctat | agcttataca | atgccttatc | cttctgattt | ccaatgatta |
| 149161 | atcactttct | tttctcttat | ttaaattatt | ttcaaaatat | tactgaaaca | tcctttaaag |
| 149221 | agtttttctt | tcagggaaac | tcataaaaat | ctatctatgt | tcactgaaac | aagcttactt |
| 149281 | tcagtataag | accttacttt | tttgggggca | aattattcaa | tcattgatac | ttctctttat |
| 149341 | gaaatctcct | aaattgcaga | tgatttggct | tcagtgtatc | tttaatgata | gaaatcacgt |
| 149401 | ataaattggg | tttaaaattc | caaaggataa | ttttgaggac | tgcagtacat | ttggccatta |
| 149461 | gagagtctag | aaatgccaaa | ttgaccaggt | cagaatgaat | ggtcttgggc | tgccatgttt |
| 149521 | catggcaacc | tgtatgcaga | aacttaccat | cttcaaatgg | gcttgttcag | atgctgggaa |
| 149581 | ctgctttagg | ccattccaaa | tcaataagac | agccaattat | acttgagtta | agtaaaataa |
| 149641 | taagcaagaa | ttgcaagaag | ccctgtccaa | gcacagtttg | aaaaacttcc | atacagagtt |
| 149701 | cttaaaagca | gactgtaact | tctaatgaat | tacttgcttc | attattactg | ctgcttgaat |
| 149761 | caagtcaagc | agtttagcca | ggacttcaca | acatgtctct | gtcactgcgt | tgatacatag |
| 149821 | taagttgtga | atggcctgcg | atgtttcttg | caatgatttt | catcatgccc | agccagtcct |
| 149881 | gccaatctgc | aaatgttcag | gcccccacat | atggaccaca | cagcccatgc | aacgcagtca |
| 149941 | aaatctgtta | gtcacgggat | gatccgtctg | gtattttcag | ctaacttaac | aattaattgg |
| 150001 | gtagcatctg | aatcaataat | tcaatgatcc | agtttggtca | gcaatgtgtc | caggctttcc |
| 150061 | tgatgattgg | tcgacttgtt | ttgattttca | ggacccagtc | acagaagtag | tgaagtttta |
| 150121 | actaggcttt | caaaaatatt | catacaactc | agctatatgg | aaataattca | tagaagcata |
| 150181 | actctgccaa | tgataagagt | gagaagccat | taactcaacR | aatgagtgac | ttgttattca |
| 150241 | catgtcttac | taaattggca | actatggctt | tctcattttt | atgttattct | atgtatatac |
| 150301 | atgtatcgaa | atagtgttat | ttagagatct | taaacatgtg | gacaaatatt | ttgatagagt |
| 150361 | ttggggaaaa | cagcctatat | ccttgcttta | tagaaaaaga | gttttcatga | gaaagaagtt |
| 150421 | atatttattc | agatatatat | gtatgagtac | atatctagat | ctatataggt | ttatatacat |
| 150481 | atataataat | atacttgagc | cccactcttt | cattccacta | ccacttcgta | gccaaaagac |
| 150541 | agagatctcg | atataataaa | ttaaatatta | agatatatat | taaagtatac | atataatgta |
| 150601 | attagataat | aaagataaaa | ttatttgaaa | tgcctcaaaa | ataatacaga | ttttaaaaga |
| 150661 | aaatgagagc | ataaaaacct | ttaaaatgta | tggatcaaca | aagaaataca | ttgcaaaatt |
| 150721 | attacttaat | tagaaaatcc | cttttaaatg | ctctttgatt | ccatgagact | tttcagctga |
| 150781 | caaagggctt | agccacctct | taaagcagag | gctctctcca | atacccgaag | ataaatatct |
| 150841 | accaggagct | ttttaaacaa | tgcttgtcat | gtttgtcatt | atgaagcaga | ctcatacata |
| 150901 | aattaagttt | tcctaaactg | tagctatgaa | ttaagagtcc | cacgatttgt | gctattgcta |
| 150961 | acctactccg | gtggtatgtt | gccatcactt | caattgtttt | aatgataaat | ttcctttaga |
| 151021 | aaatggaagg | atctgttgac | agacaaaaac | aggaactaac | aatggactat | ttagacttgc |
| 151081 | aaatgatgga | acatttaatt | attccaaaat | agcattcccc | agttagggta | tgatcattaa |
| 151141 | atccatcatc | cttcagttta | tatgcatgca | acatttacca | tgtctacctc | aaacaggagt |
| 151201 | cagtcatcca | aatatagatt | tttggaccat | ccatttcagc | atgacatttc | atcgaaagcc |
| 151261 | tggaatttca | tttattttct | gtaacagaga | taaagtgttt | gtaaacgatg | tgataagacc |
| 151321 | ataaagagaa | agtaaagtga | aattcagctt | catacagaga | taaatacaga | ccctactgaa |
| 151381 | catagcagta | gtagagaaag | agaaaaatga | gagacaatac | gtcagcagca | acattccagg |
| 151441 | caagaatcga | tgttccctac | atatggtttt | taaaaatgtt | tgctcagttt | tagttctctg |
| 151501 | aatttgcttg | caaaataatt | tgcatttaga | ctcaagacag | tggcggcaag | atcaagacaa |
| 151561 | tctaaaacta | aatatacttg | aactcttttt | gtcaatttcc | acatactgaa | aaaaaatgtt |
| 151621 | tatgtctcat | tggagagata | aatgtctcag | aaaacagtgt | tttataaaat | tcagaagtta |
| 151681 | attatttttc | tgcaggtcct | cgtttttcag | gaagaatata | gagtagctag | gtattcatta |
| 151741 | tttgaagtct | aactggagac | aagttatgtt | taggacctgg | acaaacattt | catgagatgg |
| 151801 | cagstacaaa | taaaaaccaa | ttaattctcc | tccagcgatg | tttctttcct | ctaatggaag |
| 151861 | gcttgtggtg | tgcaagaggt | gtttgggaag | gatttacaag | caagttttcc | atgaaaggac |
| 151921 | attactaaca | ttattaacgg | cccctctcag | cccgggtcct | tctcttcacc | atcattttgt |
| 151981 | agcccaggat | gacaaggcct | agagaagtta | agtgactgac | ccaaggccac | atgctgatgt |
| 152041 | caaaatcagg | cctcgagctt | atgtttcctg | acacttactc | aaatgtaatt | ttatgccacc |
| 152101 | attctgccag | tgggaaatgt | gtgtgtctgg | ccatgtgtgc | atacactcta | ccaaaatgat |
| 152161 | gtccatgaaa | ttttaaatcg | caccatgaaa | atgagaagta | agcatctaat | atcctatctt |
| 152221 | aatcaattgg | accaaattta | gaaaaaattt | gaaccctggc | cgaacgccgt | ggttcatacc |
| 152281 | tgtaattcca | gcactttggg | aggtggagct | gggcggacca | cttgaggtca | agagttcaag |
| 152341 | accagcctgg | ccaacatagc | gaaaccccat | ctctactaaa | aatagaaaaa | ttagctgggt |
| 152401 | atggtagtgt | gcacctgtaa | tcccagctac | ctgggaggct | gaggcacaag | aatcacttga |
| 152461 | acccaggagg | cagaggttgc | aagtgagcca | agattgcacc | actgcattcc | agcctgggtg |
| 152521 | acagagccag | acaccatctc | aaaaaaaaaa | aaaaaaaaaa | aaaaaaggaa | aacaaggaaa |
| 152581 | gaaaagaaaa | aaaaatccca | agctgttatg | aaaaatagtt | ttagacactc | tatgaaaggt |
| 152641 | acagaattcc | tcttttcttt | gaaatacttg | tgaatagggg | tgtgtgtgtg | tttgcgcatg |
| 152701 | cacgtacatg | tgtatacata | catatacaca | tgctatatgt | accatatata | tattgaaaga |
| 152761 | gagaatgatc | aggagactcc | tatttaaagt | atgcttaaaa | acttcagtca | cctataacca |
| 152821 | tcctaccctc | aggcctggcc | ttttgaaacc | agaaagactt | tttccttttc | cccaattttc |
| 152881 | tgtcctattc | agtcccccga | cctctgagcc | ttgtcagtga | tggtggatga | caagaaagaa |
| 152941 | atatgttaga | tgccaggtgg | tggagtaggc | attcactctg | tttgactttt | ttctattgga |
| 153001 | tctacaacat | agaaatagac | aaatcacaca | cctacggagt | tgtctttagg | atttgattag |
| 153061 | ctgacatgtg | accctgctaa | gagccagcaa | actctcctca | tcctatctgc | cctcttcatc |
| 153121 | ctggcagaag | aaccctggtt | cattatttct | gagaaaaaca | atcacaagta | gctccgtggt |
| 153181 | tgtcctggtg | aagatagaga | aatgggagag | gtatcttttt | ccttacaaag | gaaattgata |
| 153241 | ttattacaga | tagatgatat | aaacttttaa | atgccatggt | gatatagctg | cctgattcct |
| 153301 | aaataaagaa | tataaataaa | taacaggagt | atagttggtg | gcataagcaa | aactcaccca |
| 153361 | ttttccctta | aatgggctgg | caaaccaggg | ctgagaggag | acagtcgggg | agatttaaac |
| 153421 | taggaaatca | gagaaatgct | gcaggaagac | acaactcact | ccataatgca | tcctatttac |
| 153481 | gaggcaaata | aatatcaatt | gtgtacaata | ttgccatgtc | tacaaaagca | aaactcccat |
| 153541 | cattttgaca | ggtgttttgc | atacatatga | actgcaagat | caagcttgat | atgtgtgatt |
| 153601 | tcttacaata | atcagtatct | gaatcttctg | gagttggcat | gcagcactct | gtgaaagatg |
| 153661 | tcacatttag | actctgcctt | acataaaatg | tgtgcaatgt | ctatgcttaa | ctcccggcac |
| 153721 | ctgtctctgc | cttattttct | ctgcccactt | aaaatatctt | ataacaatgt | ccccatgttt |
| 153781 | accatttttt | ttcatacgca | gagttttgaa | cgtggtacgt | ttgaaagttt | tgtaggaaaa |
| 153841 | atcttttgca | tggtgaaagc | agtttatagc | aaacctacat | atctacagaa | acacttgtat |
| 153901 | ggaaaaaaga | aaaaaagaag | agtgctgcca | gacatttcac | agaattcagt | gctgcaatgt |
| 153961 | acccaacatg | gatgaaatct | ggtcccagca | aaacaggatt | tctttctgtg | agaaaatccc |
| 154021 | gcggcatata | gttccccagc | ttcctttata | atgtatcttc | cttcattccc | caatctcctc |
| 154081 | cccttcaatg | gattcaactt | tcaattttct | accatttccc | tgcaaaatgt | ctagaatcta |
| 154141 | ttctttggct | tctcataatt | cctgtcctga | ttccatttcc | acaccatttt | ctccaacctc |
| 154201 | atgtctcttc | cctttttttt | tttttttttt | tttttttttg | gcaaaaggtc | accgtgaagg |
| 154261 | caaaataggt | tagcgagaag | gcagcttcta | ttgggctatg | cttcaatttt | tctcttttga |
| 154321 | gtttctccct | acctccttgg | ccatttcctt | aaacctgtct | acacagctgt | acaccactga |
| 154381 | aagtggggtg | ttgatatgcc | aaagaaaaat | gggcaaactc | cgtgtgccag | cacaccatca |
| 154441 | catgaacaga | aaatgagaaa | ctgaaaatac | atttggtgaa | ggaattcagg | aaactgaggg |
| 154501 | acaaattgtc | acaatgaaga | tttaccccct | tggtgtcaac | acacccagga | agatcaaagt |
| 154561 | gaaagacgag | gtagaagcta | atgtcagccc | acagaagctg | gatgtgagca | cagaggcagg |
| 154621 | caggccaatc | atgggtagaa | ggatagagct | ctctagggga | gctaagttca | gagcccagag |
| 154681 | atggcgaaac | ctgagagtgt | tttagaacca | gaaaggcact | gtgacatccc | tgaggtaatt |
| 154741 | taggRtgctg | taagtagaag | cggaaaggaa | taattagagg | aactacattc | tattcaggtt |
| 154801 | tgtaggaaga | ataggatgga | gagagtcaga | gacttatgaa | ggccctcatc | ttcatcactt |
| 154861 | ctagggggct | gatggcttca | aagggatgga | atccaacatc | tgacaaatcc | gggtgtttat |
| 154921 | cttgctccta | ggaagtaagg | atggttcttt | attccttcaa | acttaaggat | attacttttt |
| 154981 | ctctccttaa | aatcaaggaa | attaattcta | cataaaaata | attatatata | atatgtgtat |
| 155041 | aaataacata | catattcata | tagattatat | atatgtatac | atatatgtat | gaatttgaga |
| 155101 | aataaataaa | ggaaaatgaa | ttaattcaaa | agcaaagact | gactgtccag | acaaaataag |
| 155161 | ctggaagatc | attaagcaat | ttactccaga | gagtgttcag | atattaccat | taacacagaa |
| 155221 | atataggaca | ctgacattct | tcataacaga | tgctcttgaa | aaatgaaggg | tttgaaaaaa |
| 155281 | agaataagga | aaaatagcta | atatatgata | aacagctcat | agaatcagaa | agaaatacaa |
| 155341 | ataaaaaaac | tgtcctttaa | aaaatatgga | aaatttataa | acttgatttt | cttcaaaaat |
| 155401 | cattagtttc | agagtactac | aaaaatattt | ttagtctaca | ctgtatgaaa | gacatatatt |
| 155461 | ttcagataat | aacacatgga | ggcaaattag | ctttgtgttt | gcatcttgca | gacattgcag |
| 155521 | taaacattta | gccaccaata | tcaataataa | gtttgtttta | ttcagtataa | tagtcttcaa |
| 155581 | aacatagacc | tgtgtttaca | gatcaagatt | aaatcaaggg | gttattatta | accctggtag |
| 155641 | tgacctctct | ttgctgaaaa | tcaaataaat | ctaacaggca | tttcttttaa | ctaagccctt |
| 155701 | gaaagagaga | ggttctatca | gtgtttgcaa | agactgttaa | actagaacaa | gaattagcta |
| 155761 | tttgtttctg | gtttactttg | ctttaatatt | ttaatggtgc | tataaatttt | ggagctaatt |
| 155821 | aacataagaa | aaagttccat | ccagtgtttt | aaaatatttt | agaacattct | ataaatagtt |
| 155881 | tcagacaatc | ttctgactca | tgtttaaaaa | catgtccaat | ttatatcaga | ataactcaaa |
| 155941 | attgcaaRta | acaccacctt | ccacccagaa | tcacttctgc | ttaagacaca | gtaaaagtaa |
| 156001 | ccagatttac | tcccctgcct | gaaacaacct | gttataatct | gtttagtctg | ctataacaaa |
| 156061 | atagcttaga | ctgggtaatt | tacaaacaat | agacatgtat | tgctgatagt | tctggaggct |
| 156121 | gggaagtcca | agatcaaggc | attgacagat | ttagtgtctg | gactgggaca | gttcctggta |
| 156181 | gatggtatct | tctatgtatc | tttaaatggc | agagggagtc | aaaaagctcc | cccgggcctc |
| 156241 | ttttataagg | acactaattc | cattcattag | agctctgccc | tcatgaccta | atcacccccc |
| 156301 | aaagcacctc | actgcttaat | attatcacac | tggggatgag | ttttcaacat | atgcattttg |
| 156361 | ggggttgggc | acaaacattc | aggacacagt | gcaaacaaaa | acaaaaacag | atggacaaaa |
| 156421 | catatgaaac | aacaattttc | aagacccact | ggacatatga | caacaatgat | ccctgagaga |
| 156481 | cggtagacaa | atgaggtagg | ccctgtgatt | gtcccagcta | ctactctagc | ttacttacaa |
| 156541 | gccatgttga | gggaggggag | agagatggag | ctgagagtcc | ggggagacca | aggaggagag |
| 156601 | agagagagag | agagagagag | aaagagagaa | caaactctgg | gagttctgca | gagggtcccc |
| 156661 | cttcagtatt | tatctgagta | cagaccaaac | tgtgcacgtg | gggaaactac | ctaaagccag |
| 156721 | aggggaaaaa | ctacctaaaa | ggatgaggag | ataatgcaat | gtaataatga | aaaatattta |
| 156781 | gttgatcaaa | cacaaggcaa | gaacacaggg | aaaagggagc | aaagaatata | caggacaaat |
| 156841 | agaaaacaag | tagaaagata | atagattcac | atttatccac | actgataatt | atattaaatg |
| 156901 | taaatggtta | aaatgcataa | gtcatcagct | tggaccagaa | aagaaaacaa | gcctcaacta |
| 156961 | cagttgccta | caagaaacag | actttaaata | taaaggcaaa | aatagtttaa | aagtaaaaga |
| 157021 | acttttactt | tcaaagttct | tttaaaactt | ttaaaaatat | accaaaatat | accatgctaa |
| 157081 | aactaatttt | taaaaagtgg | aagtgactat | atcagtatta | gacaaagcag | atttcaaggc |
| 157141 | taagactatt | aagggatata | aaagagtgca | cacagaatat | ttaYcaagat | aaaccgtatt |
| 157201 | ctgtctcata | aaaggtctca | ataccgttaa | aaggattcaa | gttatataat | gtatactctc |
| 157261 | tgaccacagt | gaaattaaat | tagaaaccca | taacaaaaag | atgtttgaaa | aatagtcaag |
| 157321 | tcattggaaa | ttaaatatac | ctgtatctat | taaataatta | tttgttgttg | aaaaccttcc |
| 157381 | cacaaagaaa | gttctgggct | cagatggttt | tgtttgtcaa | ttctaccaag | tatttaataa |
| 157441 | aaaataccaa | tcctacacaa | aatcttttac | aaaaagagag | aatatttccc | aagtaatttt |
| 157501 | ttgaggtcag | tattactctg | ataccaaaag | caagcattac | aagaaaaggt | aagtatagta |
| 157561 | tgatatccct | aaagaacata | catgcaaaat | tgtgcagaat | tttagcaaat | acaacaaata |
| 157621 | cataaaagca | taataaatgc | agaattttag | caaatacagt | aaatacataa | aagcataata |
| 157681 | tgttttgaac | aggatagttt | gtcccgggaa | tgctaagtgg | atttcacatt | caaaagtcaa |
| 157741 | ttaatgtgat | tcatcacatt | aatgagctaa | aaaagataaa | atcatctcag | tggacataga |
| 157801 | aaaagcactt | gacaaaaatc | caacatccat | tcttgttttt | ccaaaaaaaa | aaaatttcag |
| 157861 | cgaactcaaa | attgaagaga | tttttctcaa | cctgatagag | ggcatttatg | agaaatctat |
| 157921 | agctaatatc | ttcttttaat | aaatgtttcg | aaaattaatg | agatcagtgc | aagcaatggt |
| 157981 | gccctgggtc | tttgagaaag | tagagctcac | gtcagctgcc | tgatttggtt | ttgtgcctac |
| 158041 | taaatttttt | agatcctagg | ctagggtttt | gtgaaatgct | ttatctagtg | taactggcgc |
| 158101 | ttcctgctgc | cagtctacat | tcattcgtct | ttagcctgca | ccacccttaa | ctagggaggc |
| 158161 | cgatggctgt | ctctcctcag | gttaactgtc | tggggatttg | gcctcagaga | caacacagac |
| 158221 | gtaaagctcc | ttgggattcc | ttccatcatt | tgtgttctaa | atgattagta | agagtgcagt |
| 158281 | ctaattttta | ttgtcatgca | tgacttgggg | tacagtcatg | ctgaaccaca | taaatcttta |
| 158341 | gagccaatca | ttgcaggaac | tggtttgact | gtttccacaa | gtaaWctcgc | cagcattctc |
| 158401 | aatgtgggct | cttgaatcat | cactcctaaa | tttctgagct | catctgagct | ttgcgataat |
| 158461 | aatagtcata | attctgaatt | taatatagat | gctttcatct | gcaaagtgct | tggcaaacat |
| 158521 | taactaatta | gaccccggat | gctcctcttg | aggtcctcaa | acaaatagca | ctgcctcatt |
| 158581 | tttgaaatga | ggggtaagaa | aaagacctga | agctcagagc | tcattagggt | agcacaaaag |
| 158641 | acagtgggaa | tcaaacctag | aacctgctct | cgtcacatgg | gcttgggtga | catacctcca |
| 158701 | acagaacact | ttcctggaat | cattaaccta | acttttcttt | tgcaaggaac | gtgcatgaca |
| 158761 | aatgtggtta | ctttaaaatg | ataggatttc | tttttatgag | ttattgaagt | tttgcactga |
| 158821 | atattccttt | ctacctaaag | aattaagccc | cacatcttga | gtcatgcttg | gaatggtatt |
| 158881 | aacctcatat | tttaaagagg | cttctcctag | ggcttatggt | gaaacaagaa | tacctatgca |
| 158941 | caggccacag | ttatgtcagt | gagcataaaa | attaagagtg | cctgcaagtg | cctagctgca |
| 159001 | ggtcttcctc | atcccagcaa | tcaggtcatc | tgcacaatga | gatacggagg | ctatctgagt |
| 159061 | gtctgactaa | actctgccaa | agctccttta | tttgttaagc | ctaaatttaa | tattcccctg |
| 159121 | cctccaaatt | aattcaccct | aataatttgg | aatgtccttc | acttttgctg | cctcctttat |
| 159181 | gaatcaatct | agagatcaca | agtgaaaagc | taacattact | attgcctctg | tgtaaaagtc |
| 159241 | cataaataat | acttcttcat | tatcaacaaa | gagtatttgc | tatccagttc | agtcatgagg |
| 159301 | tttttataat | taggaaggga | atctggtaat | ctcttgtatt | tatttgctta | cttttatgaa |
| 159361 | tgtagcaaca | attgtgtgcc | aggctattga | gaataaagtg | tgtgggacag | agacatccac |
| 159421 | cgattcatgt | gaacaccaac | aagcgtttgt | ctgaatctgg | ccttgaatgt | acttggtggt |
| 159481 | ttccttccct | gccagcagta | aatttcaatg | cttgtaacaa | atgatcttta | atggcttcat |
| 159541 | tgcaataaag | gcagcccatt | ttgttctgtg | cagccagacc | caSgtcttgc | ttaaagtagg |
| 159601 | ccaccttccc | ttggagcatt | tatcccacta | tttcaaagtg | cttggtatat | attatagaac |
| 159661 | tttctccatg | gttttagaag | acatctgaac | aatggttaag | aacaaagttt | ttagatggga |
| 159721 | gaactgactc | agcagatctc | ctcccaaggt | cagcctcatt | agcagccaat | cagatgtgtg |
| 159781 | cctgctgctt | gttcctttgc | atattcaaca | catatttatc | atgtatttat | tatgtggtac |
| 159841 | agtacaggca | tacttcctgc | cctccgagag | tttacagcct | ggtaagaata | tgagatagtc |
| 159901 | aagtaataat | gtataaaacc | ctattagaat | aaaatatgct | gcttcgacac | ttaggagagt |
| 159961 | gactccattc | cagtttgggg | catgagggat | gatatgggac | agtaactgct | ttctacactg |
| 160021 | agatctgaaa | ggcagtatgt | gttagtaagc | tagagggaga | ggcagaaaag | agagctgcag |
| 160081 | gccaacgaag | tagcttgcat | gaaagcctgg | aagcaacaga | gtgtgcagag | tgatggggtg |
| 160141 | agtggatttc | actctatcag | gaccaaggta | ggaaaagaga | aggcaggaga | tgcagctata |
| 160201 | tatatacttg | aaccaggttt | tgctggtttt | gtaagccatg | gtaaggactt | tggacttttt |
| 160261 | cctgagagta | atggggaaaa | ctttgaaggg | cttgagcaat | agcatatcag | gactggtttt |
| 160321 | gtatctttta | aaatatcagc | caggtgcagt | ggctcacacc | tgtaatctca | ccacttggaa |
| 160381 | ggttgaggca | ggaggatcgc | ttagggccag | gagttcaaaa | tcagctgggc | aacacaacaa |
| 160441 | gacctcatcc | ctacaaaaat | aaaaaagaac | ttagctgtga | gtggtggtgc | acacctgggg |
| 160501 | tcccagctat | tggggaggct | gaggtgggag | gattgcttga | gcccaggaag | tcgaagctgc |
| 160561 | agtgagccat | gatcacacca | ctgcactcta | gcctgggtga | cagagtgaga | ccctctctca |
| 160621 | aaaaaaatct | ttttaaatca | tttgtagagc | agagtggaga | aagattgttg | ggcagaggcc |
| 160681 | attcaagagg | ctgctgtagt | aacgcagata | tgtcatgatg | gtggcctaga | ccagaaaagt |
| 160741 | agcaatgaaa | acggagaaaa | tggaatggac | ttagagaaga | tacaagagtt | aaaattaaga |
| 160801 | ctttcagatt | gttgactatg | gcaggtgaag | gagagggcgg | agtcaaagcc | gactcccagg |
| 160861 | tttcagctgg | gttgatgttg | gttctatttc | ttgatattag | gagcacaagg | aagaatagat |
| 160921 | gagagtagga | aatgaggatg | agaaatgaaa | atttcatttg | gtagtttaat | tctaaactgc |
| 160981 | ctatggggat | ggcctgtaga | cagctggatg | catttgtgtg | aagtctagga | gagagagaga |
| 161041 | gactggggat | atgggtatgg | gcatcattac | tataaagata | gtagatgaac | caggaaattg |
| 161101 | ccccagggag | gaaaagaggg | gatccttgga | taaaatcact | gaagaatatt | gtcatataag |
| 161161 | gggcgaagag | gagtatgcaa | ggaagactaa | ggaatggccc | gagaagtaca | aagaaagcca |
| 161221 | agagagaaag | ataccacaaa | agcccaggca | acggtgcttc | aggaaggaca | agaggaagtg |
| 161281 | aatcaataag | gaactgaaac | atatgaacag | gatttaggaa | aaaggttgtt | ggtgtctcta |
| 161341 | gtgaggacat | ttcattttag | tggaatggtt | ataaaataaa | ccatacgtct | gggagctgaa |
| 161401 | gagtgagaaa | atatagacaa | cacgtgtaga | ccttttttga | gaaattggga | agaggaggag |
| 161461 | agaaacatgg | tattagctga | aaggtgagga | ggggtggata | gctgacgatt | ttacaatgcg |
| 161521 | aaagagccga | aaacatttaa | ctgcttacag | agaaggcaag | gaagagagaa | gagaaagagt |
| 161581 | tcaggatgga | gagcgcatca | tttatgaagg | aagcaggact | tgtacctcca | gcacttgtaa |
| 161641 | ttttagaggg | tgtccttgac | ctcaggttaa | atattaaaat | gcataccgag | ttcacatcaa |
| 161701 | ctacatgttt | tacaacaaaa | aaattaaaag | ggtttttata | attttgtttg | caaaattagt |
| 161761 | tggctgtgaa | taactcacaa | tttataattc | aatatgactt | atcagtaatc | aacgcacata |
| 161821 | cttgggaatt | tgggaaaact | gagaacatcc | aactaataaa | ttgttttcaa | atgtagtaag |
| 161881 | acatttatgg | atactatgtt | tacaatgctg | acataatatt | acattttata | tgtactctat |
| 161941 | tattacatgt | gattttcatt | attcacattt | tgaatttaat | attctttttt | ctggatgcaa |
| 162001 | aaatgtctac | aggtacttgg | ctcgttcacc | agtcccaggc | agtttactta | tattgttcaa |
| 162061 | tggctgaaat | tctaaagaac | agggttcctg | ccatgacagc | agagtgaagg | gaccagtctt |
| 162121 | caggaggagg | agactatcct | ccttttgtac | aggagaaaag | cacaaaagaa | caggcacaga |
| 162181 | Sgcaagctgg | taaagtgcca | atctcatggt | ctccattttc | tctattgagg | tgccatcacc |
| 162241 | tctttgtgtg | tgtctgtagt | gtgtgcgtgt | gtgtgtgtgt | gtggtgtgta | tagtatatgt |
| 162301 | agatgtctgt | agtgtgtata | tgtgtgtatg | tttgtagtgt | ctgtatgtgt | gcatcctgta |
| 162361 | tgtgtatagt | gtgggtgtag | tatgtgtatg | tagtatgcat | gtgtgcatgt | gtatagtgtg |
| 162421 | tatgtgtgta | aatatatata | tatttatata | tggtgtgtat | atagtgtgta | tatatgtggt |
| 162481 | gtgtgtgcat | gtgtgtagcg | tgtatatgca | tagtatgtgt | atgtgtggtg | tgtgtgcatg |
| 162541 | tgtatagtgt | gtgtgtagtg | tgtgtcgtgt | gtgtatgtgt | agtgtgtgca | gtatgtaggt |
| 162601 | gtgtttgtgc | atagtatgta | tatgtgtgtg | tagtgtRcgt | atgtgtctac | gtgtgtgtgt |
| 162661 | gtagtgtgtg | tgtgagtagt | ttatgtgtgt | agcatgtatg | tgcatgtgta | gtgtatgtgt |
| 162721 | gtggtgtgta | tatgtgtcta | tgttgtagtg | tgtctatctt | cttgagagat | ggggcattga |
| 162781 | tattcctttt | agaaattctt | ctagaagcca | acatcttgaa | aatgagcctg | ctgatggtat |
| 162841 | tcagagctgg | ccagctcttc | cccattctcc | ttcaatccgg | gcacgtccca | ccctgtgtgg |
| 162901 | ctggatgtga | acacgtacgt | gcaagtgtga | tctctctgtg | tgaacattaa | gaggttgctc |
| 162961 | acttgctcca | gttttcacct | ggaagcagca | cttccttccc | cattccctcc | tattcgatgc |
| 163021 | aacaggctga | tcagagtatt | tctgttcacc | tttgtggcaa | ctgtgctaca | ctcccctttc |
| 163081 | aagccttcct | caaaagaaag | aggagaaaat | attttaatat | gattttaatg | cctagttttt |
| 163141 | tctcctttcc | tttttattgt | aaaaatacaa | cacttttcct | gtagagcaat | tagaaaatgc |
| 163201 | agatgcaatt | ttaataccaY | cattgMcaca | tcaaccatgt | tactatgttg | ctttttcttt |
| 163261 | aaaaaaactc | ttgatagaag | cagtttttta | aacttttatg | tatttaatta | ttttgcaaga |
| 163321 | tttcaatttg | cattgaaatt | tattgagatt | tattttatgg | cccagcacat | gacttatttt |
| 163381 | ggtgaacata | ttctgtgcac | ttaaaaatgt | gtattctgta | attggtgagt | gagtttctag |
| 163441 | gaatgatgga | gttcaagaaa | tgttcatcag | gtcaagttgg | ttggaagtgt | tcgacagaag |
| 163501 | tcattttata | acctgctttt | ttattttgag | taaaatattg | tgacagtcaa | taaacatgat |
| 163561 | ttttctacaa | aatgctttat | agttatggtg | tattccatgg | tttagctatt | tccctattgc |
| 163621 | tggatatttg | ttttaaaatt | tcggtattat | aaagaatact | gtgataaccg | tacatttaaa |
| 163681 | tgtgtatgta | ttattgatta | cactacagaa | gataaacttg | tagtagttgc | tatcatttga |
| 163741 | atgcatcccc | aaaagttcat | gtgttgggaa | ctcagtcccc | aatgcaacag | tgttgggagg |
| 163801 | tgagaccttt | aagaggtgat | tagagaaatg | gattaatgga | ttaatcttgg | gcatgggttc |
| 163861 | attttcttaa | gtgagtttgt | tatgaaggca | agtttgggct | gggtgtggtg | gctcacacct |
| 163921 | ataatcccag | ctctttggga | ggccaaggtg | agagaatttc | ttgaagccag | aagtttgaga |
| 163981 | ccaacctggg | tgacatagca | caatccccct | ctctaaaaaa | aaaaaaaaaa | tcagaaattt |
| 164041 | agccaggcat | ggtaacgcac | acttgtagtc | ccagctactt | gggaagctga | ggtgggagga |
| 164101 | ctccttgagt | acaggagttc | aaggctgcag | tgagctatga | tcatgccact | gcactcctgc |
| 164161 | ctgggcaaca | gagtgagacc | ctgtctcgaa | aataaaaata | aaaaaataaa | aaagcaagtt |
| 164221 | cagccccctc | ttgcttttgc | tctcttgcct | ttcctctttc | caccatggga | tgatgcagca |
| 164281 | acacatccct | caccagatgc | tggcccctta | atcttggacg | ccccagcctc | cagaatcata |
| 164341 | ggaagtaaat | cattgtcctc | tataaattgc | ccagtctgtg | gtatYctgtt | atagcagcgc |
| 164401 | aatacagact | aagacagcag | tgtagctgct | agttaagact | tcgtattctt | gaaggccttt |
| 164461 | gattcatatt | cattttgagg | ccattttaac | attgtacctt | tacccttctg | aagatctctg |
| 164521 | gtgtcttggc | atgtattttc | atcactttct | atatagaaaa | tcagtcacag | gtgactcact |
| 164581 | gaatttggag | ccagcatgga | aacaatcaaa | atgccaaaaa | tctaactttg | taaaattaaa |
| 164641 | aaaaaaaaat | ctgcctcttt | aaaaatttga | ttcacttctg | ttcttaaaga | tagcaatgcg |
| 164701 | agatggtgtt | caactccaaa | actgcaaaac | actgatgcca | agcaatccat | tttaatcatg |
| 164761 | gcatgccccg | ggacactttc | agtatttgca | cgaattgaag | gtcatttgtg | gtgagtacaa |
| 164821 | ggtcttctct | cctgaatcat | cgtgccattg | tactcacctc | gaaaatgctg | ggatgcctgt |
| 164881 | ggggatcctg | aaagcatcag | ctggacatga | tgattaaaat | cttctaatta | tttgaaaagc |
| 164941 | catttgggat | aatagagtag | gtttactttc | ctttcattaa | tgtccctgag | caggctatat |
| 165001 | aggagttaga | aagtcacaac | ttcactgctt | ttggccaaag | gccattgcgg | tacttgcaga |
| 165061 | tgaagaagaa | aaagaaaaaa | aaaaacagag | acaaagtgtt | ctttcagaaa | ggttataagc |
| 165121 | ctgtagacag | aacagaatta | aaatacaatt | ctgaatgtac | cagcatacaa | tattttggtg |
| 165181 | ttggtttact | gaagagaatg | tcagatcaat | acaccaccaa | gtcaaattga | ctgtgatgtc |
| 165241 | taaaataact | ttaggtctgc | ctttagaagg | agctaatggg | aaaagagaga | aataagttac |
| 165301 | acgaaggtcc | atgttgggcg | ataaggaact | tgtcgcttag | tgaacaggtc | ttgataatat |
| 165361 | actggataaa | tggcactaag | aaatgtctgg | aatcactcag | aatacatgcc | tagaagtcta |
| 165421 | gagtaataag | aaacaaacgc | atgtcataat | tcagtgtaag | aaatagcaat | gcaaaggtaa |
| 165481 | gatctttgca | ttgtataaat | tcgtataagc | cctgtaaatg | attacaactt | catttcttat |
| 165541 | catttgtttt | ggatagcaga | attagagtag | agcagtgtta | gcaggagaga | gtcccttgaa |
| 165601 | ggtcacaaca | atgtgaagag | cctggcagtg | tggagtgtca | gctgaataac | ccatgaggac |
| 165661 | ccaaggaaca | ggcctggatt | tcttccacag | agcaggcctg | gcgaattccc | atgaagttag |
| 165721 | gtcactaggc | aaacgttcca | gggctggcgg | tgtacttatg | actctcaatc | tcccttctaa |
| 165781 | gttgttaaaa | aaattaaaat | aaaaaaaaaa | ctcttcagta | cttagacatt | atgctaaggc |
| 165841 | agtatctgaa | atgacagaat | ggaatgaaga | gacatcagat | gaatgctaag | tatggagact |
| 165901 | atgcagagtt | tagaagttgc | cctgaaaatt | gagagaagct | tagggttggg | gctaatggat |
| 165961 | gaggtcagaa | gcttatcaat | cacccaatta | acaagtattt | attgtgacca | tacaggatac |
| 166021 | tggatgccac | agtaactgca | tatggtgaga | ggtgtaagac | ctaagttgtc | taactgtgtt |
| 166081 | taattcattg | ggaaatttac | taacaggtca | aacaggctta | ccccagtgaa | ataatccacg |
| 166141 | tggcatcagg | tcacacattt | ggaaggtgac | cttaaatata | gatgaaggaa | attgttgaag |
| 166201 | tcagtggcca | gtatattgaa | ttacaaggct | cataaacttc | tcatgcctca | gatctcgagt |
| 166261 | tctattttta | gagacacatt | ttctggcaca | tttgttttaa | aaagtctctt | ggtggggtgt |
| 166321 | ggtggcccac | gcttgcaatc | ctagcacttt | gagaggccaa | agtgggagga | ctgcctgaga |
| 166381 | tcagaagttg | gagaccagcc | tgggcaacat | agcaagacct | cgtctctaca | aaaagaaaaa |
| 166441 | aaaaaatcaa | ctgggcatgg | tggcatgcac | ttggagtcca | agctattcca | gaggctgaga |
| 166501 | cacttgagca | caggaatttg | aggctacagt | gacctatgag | tgtgccattg | ccttccagcc |
| 166561 | tggctgacag | agtgagaccc | tgtcccaaga | gaaaaaaaaa | aaaggagtct | gaacaaatat |
| 166621 | caattctgaa | ccctcttgga | aatcctacaa | tgtctatata | tttttctagc | caatggaaca |
| 166681 | gatataaaaa | cttctattgt | caaatgtttg | ttttaaatct | taagagagat | tctttagctt |
| 166741 | tgttatctgg | tcattcctca | tttagccaat | aatagtacaa | cttcctagaa | aagaatgaag |
| 166801 | atttttggag | gaactgagct | ttcactttgt | ctgcacagtg | tcacagggta | ggcccttgcc |
| 166861 | ttcccacgtt | gtggtcatta | gcatgaaatt | gcttggaaag | gtaacctatg | ctctcctaac |
| 166921 | tatgaggatg | aaaatggtat | cccataataa | aaatagaaaa | aggtaataaa | agttcacttc |
| 166981 | ttgttgcaat | agcaaatgca | caagtagcat | ttatcacttg | actgttttcc | tgtatggttt |
| 167041 | tctagtcatt | cgttgctatg | catggagaag | cgtgaacttg | tgaaaatact | tatcatctgc |
| 167101 | ttcagtggcc | cattttcaat | gcatttacag | catcaacaat | aacaacatac | aatgactttt |
| 167161 | ttttaaaaaa | aaaggtaatt | ttggacacag | aacctatact | atctttatat | ttttgtttgc |
| 167221 | tttggctttg | ttttgttttt | tttgaaccta | acaagaagat | ggtagcatta | gccactctca |
| 167281 | gttaaaaatt | tgaaaacctt | acagctagag | aaatagtctt | tcagtgactg | aggtacgata |
| 167341 | atttcacttt | acgctgtttt | agcttcatta | cattgacaac | agatttttat | atttcttata |
| 167401 | catttttaat | tattcttttc | tatcagatgt | tgagaaattc | tatatatcat | gttgcaaacc |
| 167461 | taattatttt | taaaaagaga | tttatgtcac | atatgactga | cttcttatct | ataactccca |
| 167521 | gaaatactaa | ttttatttgg | aaatacccag | aaagatctgt | taaattatga | atatactgtg |
| 167581 | actcatttta | tgcctaatta | taaaaaatga | atgagtaaaa | tatcaaaaaa | atttctttgg |
| 167641 | gaaattttag | tttaaaaaaa | gctagtaatt | ttctaacaga | gccttagaaa | taattgtact |
| 167701 | cccaatttta | tggggaagtc | atttcattat | taatgtgttt | aattctcaaa | agttagttgt |
| 167761 | gatattttta | gtgtaaggtt | gcttggaaaa | tatatgaaaa | aaagatataa | ttattatgtg |
| 167821 | aattttatta | tctctccaag | aaagcaaaaa | taaacagaaa | gactaatttt | agataaggat |
| 167881 | aagaacatct | aggttctttt | tgctctacta | ctaagcaatc | tttggcaaat | gattttaatt |
| 167941 | ttctggagct | tgatttctgc | ctctatgaaa | taaagaggct | agaagtagat | cacatttggt |
| 168001 | ggtaatactg | agacRcgctc | tgcaaaaaaa | atgcttcttg | gtcaactgaa | tttggaagtt |
| 168061 | atcatatgtt | atgcccacta | tgggatattt | aagatgcaca | ttggtagatt | atgagaatcc |
| 168121 | tgcagtaaaa | gaacctactt | atcttggttt | ataccaaaat | ttcccaaagc | tatttggcca |
| 168181 | tggacctctt | cctttgtttt | ctttctgtaa | caacatactc | ggaacacacg | ctaggaaatg |
| 168241 | ctggcaggat | attcaactgg | gtgcgcctgg | tccctggttt | tcaacacaag | atgatgaaaa |
| 168301 | gctgtgtctc | atttttcttt | tgagaaaaaa | atttaaaatt | ctatgtatgc | attaatctct |
| 168361 | gattactttt | cctaataata | tcttattttt | cctacttttt | tagtttccgt | gaacaccaaa |
| 168421 | agtcataaat | tttatccaca | cactaaggac | cacaatttct | taagtcacta | caacttctgg |
| 168481 | gacgctagca | aatcaccact | attaggttaa | gccttcctta | attactaRac | aaatttcaaa |
| 168541 | aagcacaaat | gggtaaatct | ctggacatcc | taagcaaatt | ttcacacttc | ttaaataact |
| 168601 | gtaacaaatc | agtggccaca | tttactcaac | taatatttat | ttatttatta | tttttatttt |
| 168661 | attttatttt | ttgaggcagg | ctggagtggc | acaatatcag | ctcactgcaa | cctccgcctc |
| 168721 | ccaggttcaa | gcgattctcc | caactcagcc | acccaagtag | ctgggactaa | aggtgcatgt |
| 168781 | caccatgccc | agctaatttt | tgtaattttt | ggtagagacg | gggtttcacc | atgttgacct |
| 168841 | ggctggtctc | gaaatcctga | cttcaagtga | tctgccagcc | ttggcctctc | aaagtgctgg |
| 168901 | gattacaggc | gtaagccatc | gtacccagcc | tcaacaaaca | tttaatgaag | atatgtcata |
| 168961 | cacaaaagca | tgacagatac | tctaaaataa | tgaagaaatt | ctcccctaca | ttaaagaagc |
| 169021 | tttttgtcta | taagaagatg | aatataataa | tattttagta | atataatata | tattacataa |
| 169081 | tagcaaaaat | atcaatagct | agtgtttatt | aaatgcctac | taatattgca | gatactatat |
| 169141 | atccataatc | attcatcttt | aaaaaaaaaa | acccttaaag | cacagattct | attattgttc |
| 169201 | tcattttata | agaaggaaaa | aggagctcag | agaggttagc | gatttgtctt | atgacacaca |
| 169261 | gctgtcaaat | aaaagaaagg | attcagatca | aggtctatat | gactgcagca | tcacactgcc |
| 169321 | tttcaaatca | ggcttaaaca | tgaatgtagg | cagaagagaa | ggaactaatg | agatggggaa |
| 169381 | gtagtgccgt | acgaagagga | atacttgata | caactgggct | tcataagaag | ggtcaagtca |
| 169441 | agattgctag | gagagttagt | tggccttggg | aaaatgagtg | actgtctctt | tctctgagac |
| 169501 | aggaatgaaa | aatgaaagag | gagatggacc | agaagtgtgt | ggagaaaaag | gccttccaga |
| 169561 | ctccctttct | cacactaagg | tctgggaacg | attcctaatg | tctgttctct | ccaaggtaat |
| 169621 | acgtgtttga | atgtaacctg | cgtttcttac | ttgatatgag | ccctaacctg | aatgtactcg |
| 169681 | aggtctctcc | atgaactacc | aagtctatct | aattagttaa | aatattagta | tccatggttt |
| 169741 | cttctcagaa | actcagagac | ctaagccgta | gttttttgtt | tgtttgtttg | tttctttgct |
| 169801 | actaaaaaaa | acaagcatag | acgacacacc | agaatttaaa | taactaaaaa | ctattctccc |
| 169861 | tggcttcatg | agtacctatg | agagtcaaca | cagctcccct | atcttgtggg | aacccttcaa |
| 169921 | tcgcaaaata | cccatcttgg | agagtataaa | ttgtgtgttg | gaagtttaga | gaaagggcac |
| 169981 | cttccatctc | aatctattcc | aaatgccaaa | cgtgagaatt | tagcttactt | ttgaaatgaa |
| 170041 | gtcttcattc | ttaattacaa | acatttcttc | ttgttttgtc | ctagacacaa | agattcctaa |
| 170101 | aaagatgtca | aagctggtga | gtggcagctt | agggcccttg | aagatcactg | tgtgtcttct |
| 170161 | cctgtgtaaa | ctttacctac | ttaagaggat | tgaggaaaga | gtcccctttt | aacccttcat |
| 170221 | cctctcttcc | atttataacc | tttgaacttc | atggggctta | agcaaattaa | agtattagat |
| 170281 | tccaccatga | ttcaaggtaa | tgaaaacctt | tccctaaggc | ctcagatgag | ctatagaaaa |
| 170341 | taaagaccgt | atgtgaattc | tttaggttca | tgatgtttga | ctcttcaatg | ggtagtgagg |
| 170401 | ggggctcagg | gagcatttta | tacagtcctc | atccaaagat | aagcaatggt | ttggtaccca |
| 170461 | agagctctgt | gttctagtct | cagctctgct | gcaaattaat | tgcctggcct | tgggcattta |
| 170521 | gacagatgag | gttaaatagt | ctttttaaaa | gatccttcta | gttcaaagat | tctaaatcat |
| 170581 | aaaactgggc | aaaatggaat | acctccatga | aggaaagacc | caactgaggg | tgtttgcatt |
| 170641 | tcagaatgca | atgcacacat | catttggtga | caaacacagt | tttttcactg | aacactaagt |
| 170701 | gattaaagag | tgcatacatg | cacactgaat | agacttactc | atggaaatca | cattagtaaa |
| 170761 | ttataaaatt | gtatgaaaag | tccaagcagt | gtgcattgca | atagtttctc | aactctgctc |
| 170821 | tataaaatga | tggaaatcca | tacacagcag | ttatctctaa | gagagttagc | aattaccctc |
| 170881 | aggagtttgc | aagtcaaaag | ttctagacct | ctggatgcct | gggtcactgg | ctgtggcaat |
| 170941 | cccaaacact | gtccactcca | agctttggaa | tctgcatcct | cttattttgc | aatagaaagg |
| 171001 | agcaaatgta | gtttcccaac | ttggggaagg | tccaggactc | aagaagtcta | ctaaatgtat |
| 171061 | ccaagcattt | agaatattat | gggtataaca | attcactcaa | ttaaagagaa | gggcttctgg |
| 171121 | tgtttttgtt | gtgtgctatc | atctagtaat | aatgctatat | ttagacatga | ctatccctac |
| 171181 | ttatacatct | cctacatcct | gagtctataa | acatttggga | aaatgctggc | ctgaattcag |
| 171241 | ggatgaagta | tgaagacttg | gaagaaaaga | aacgctaact | ggttaaatta | caggttgggt |
| 171301 | ctgtagtttg | gttttgttaa | atccatactg | cagtatgaga | tcaaaattaa | ctttgtatcg |
| 171361 | tagctgccat | tttctactga | tttttgccaa | gagttctaaa | tgttctgttt | tctctccttg |
| 171421 | aggaaaattc | ctatttttct | tttcctgaca | cagactttcc | gtcataccat | aggaaagctc |
| 171481 | aaaataaact | atactctaag | ctacatgagg | aaaaaaaaag | tctttgtatc | tcttgcacct |
| 171541 | ggtatgatcc | ctgatactgt | agatactcac | taaatgttca | ttgaataagg | ctatcaaatt |
| 171601 | aatgaattaa | taagagaatc | aagaatttgg | ggatcctatc | acgtaaatgc | atggagcttg |
| 171661 | caaatgtctc | atgatttatc | ttgatccttt | gtaaaatgcc | aacttaagct | tgtgttagtt |
| 171721 | ccattcggaa | actggcctcc | tagcataacc | ctaaSggata | attataaatc | cacaaaaaat |
| 171781 | ctaagaaaat | tctctacaca | atctctttac | caattcagat | cggtcctggc | agacctaaaa |
| 171841 | ttttgtgatt | tcctggtatg | gtgaattctc | atcaaatgcc | aacaagagga | tatctagaga |
| 171901 | aaaatgttct | gttacgttca | ctgaaaatga | agacagcatt | tttgtttttt | tctcattggt |
| 171961 | ttttcctaga | ttacattatt | atagctacag | atcatcccct | cctttgaggg | tcatttccaa |
| 172021 | gaaagttcaa | gtttagattt | tcccctcgat | gtctcttaaa | aagcccttct | aaaataagag |
| 172081 | atattattgc | ggtcaaatct | actttgcaag | aaataaactt | ggcatcaaag | gaaaactaca |
| 172141 | tcattcttcc | cctcaaccat | agtcagtcat | gtctatggca | atcctggatt | caattattag |
| 172201 | tgattctgcc | agcatcttga | catttctttt | agagtcagag | ggaggaggga | caaaagtgat |
| 172261 | ctatttatat | gctacctcgc | cttttttctt | tccccctctt | tttcctatat | acctaagact |
| 172321 | tcagcagtca | gtatccagat | ctcaatttct | cttccttgtg | tcaaaagtta | gggaacggtt |
| 172381 | tctgggtggg | tggaagaaca | tcactttaat | tttcaggctt | taagaatctc | aacctcattg |
| 172441 | gggataccaa | gaaaataacc | tagaaaactc | cttccacaaa | gtggatggac | acctttcaga |
| 172501 | taagcagatg | actcaaagtt | attttatctt | cacgaaagag | ttcattcatt | aaaatgagaa |
| 172561 | agttgtttac | tcttgaagaa | ttatttacac | ctctgaagaa | taattatgcc | aagctatgag |
| 172621 | aaacctatag | ggtgtgtgtg | taagaattta | tttatttatc | tgacaactgt | ttattaagca |
| 172681 | ctgattatgt | gccaggcata | gttctggttg | ttgaggatac | agtggtgaat | aagaccccaa |
| 172741 | aagtccctgt | tcataaaagg | aaaacattta | aaaaacaaat | aagtgaataa | tttagtataa |
| 172801 | ttccaggcga | tgctatgaag | aaaaagaaaa | gcagagagta | aggatgtgca | tttgtctaag |
| 172861 | agagcaacat | cgaagtgaag | accttgagag | gtgacagcgt | gctggcagtc | ctcagagccc |
| 172921 | tcgcttgctc | tcggcacctc | ccctgcctgg | gctcccactt | cggcggcatt | tgaggagccc |
| 172981 | ttcagccccc | cactgcactg | tgggagccac | tttctgggct | ggccaaggct | ggagcccact |
| 173041 | ccctcagctt | gcagagaggt | gtggagggag | aggcgccggc | gggaactggg | gctgcctgcg |
| 173101 | gcgcttgcgg | gccagctgga | gttccgggtg | ggcgtgggct | tggcgggccc | cacactcgga |
| 173161 | gcagccggcc | agccctgctg | gccccgggca | acgagggact | tagcacccgg | gccagcggct |
| 173221 | gtggagggtg | tactgggtcc | cccagcagtg | ccagcccacc | ggcgctgcgc | tcgatttctc |
| 173281 | accgagcctt | agctgccttc | ccacagggca | gggctcgRga | tctgcagccc | gccatgcctg |
| 173341 | agcccccacc | ccctccatgg | gctcctgtgc | ggcccgagcc | tccccgacga | gcaccacccc |
| 173401 | ctgctccacg | gagcccagtc | ccatcgacca | cccaagggct | gaggaatgcg | agcgcagggt |
| 173461 | gcaggactgg | caggcagctc | cacctacagc | cccggtgtgg | gatccactag | gtgaagccag |
| 173521 | ctgggctcct | gagtctggtg | gggacgtgga | gagtctttat | atctagctca | gggattgtaa |
| 173581 | atacaccaat | cagcaccctg | tgtttagctc | aaggtttgtg | agtgcaccaa | tcaacactct |
| 173641 | gtatctagct | gctctggtgg | ggccttggag | aacctgtgtg | tggaaactgt | atctaactaa |
| 173701 | tctgatgggg | acgtggagaa | cctttatatc | tagctcaggg | attgtaaata | caccaatcag |
| 173761 | caccctgtgt | ttagctcaag | gtttgtgagt | gcaccaatcg | gcactctgta | tctagctgct |
| 173821 | ctggtggggc | cttggagaac | ctgtgtgtgg | aaactctgta | tctaaatctg | acagggacat |
| 173881 | ggagaacctt | tgtgtctagc | tcagggattg | taaatgcacc | aatcagtgcc | ctgacaaaac |
| 173941 | aggccactcg | gctctaccaa | tcagcacgat | gtgggtgggg | ccaaataaga | gactaaaagc |
| 174001 | acgctgccgg | agctagcatt | ggcaacccgc | tggggtcccc | ttccacactg | tggaggcttt |
| 174061 | gttctttcgc | tctttgcaat | aaatcttgct | actactcact | ctttgggtcc | gggttgcttt |
| 174121 | tatgagttgt | aacactcacc | gtgaagattt | gcagcttcac | tcctgagccc | agcaagacca |
| 174181 | cgagcccacc | gggaggaacg | aacaactcca | gacacgctac | Yttaaaaaat | gtaacactca |
| 174241 | ccgcgaaggt | ctgcggcttc | actcctgagc | tagcgagacc | acgaacccac | cagaaagaag |
| 174301 | aaactccgaa | cacatctgaa | catcagaagg | gacagactcc | agacgcgcca | ccttaagagc |
| 174361 | tgtgacactc | accatgaggg | tccgcggctt | cattcttgaa | gtcagtgaga | ccaagaaccc |
| 174421 | accaattctg | gacacaccct | gaaggataag | aagaggaaca | tgagagatgc | acactctcta |
| 174481 | atggcaggag | acctgaagac | cctccacagc | cacatggtaa | tacgtggtca | taaaactggg |
| 174541 | caaaatggag | tacttccatt | aaggagaaac | ccacctgaga | gagtttgcat | ttcaggatgc |
| 174601 | aatgtacaca | actttttttt | ttgtgagaga | gagtcttgct | ctgttgccca | ggctggagtg |
| 174661 | cagtggcatg | atctctactc | actgcaactc | cacctcttgg | gttcaagcga | ttctcctgcc |
| 174721 | tcagcctccc | aagtggctgg | gattacaggc | gcccgctacc | acactcggct | aatttttttg |
| 174781 | tatttttagt | agagacaggg | tttcaccgtg | tcagccagga | gggtctcgat | ctcctgacMt |
| 174841 | catgatctgc | cagcctcggc | ctcccatagt | gctgggatta | caggcgtgag | ccaccgcgcc |
| 174901 | tggccaagta | cacaactttt | ggtgacaaat | agttttccac | tgaacactca | gtgattaaaa |
| 174961 | agtgcatata | cacagaatag | actcactcat | ggaaattaca | ttaataaatt | atataactgt |
| 175021 | ataaaaagtc | caagcggtga | gcatggacac | tgcacacaga | gccaggtaga | aggtgacttg |
| 175081 | cagagacatg | cacagagcaa | gggcacggtc | caaattgagt | aggtcttttt | aagccaaggt |
| 175141 | gaagttttaa | ttttatttta | agtgcaatgg | caaggcattg | aggacttgaa | cagggcgtga |
| 175201 | tgtaaactga | tttatatttt | aaaagattgt | tgggggttgg | gcatggtggc | tcatgcctgt |
| 175261 | aatcccagca | ctttgggagg | ccaaggcagg | tgaattgctt | gagcccagga | gtttgagacc |
| 175321 | agcctgggca | acgtagtgag | acattgtctc | tacaaaaatt | acaaaaatta | gccaagcatg |
| 175381 | atggtacgtg | cctgtagtcc | cagctactca | ggaagctgag | gcaggaagat | cacttgagca |
| 175441 | caggagttca | agggtgcagt | gagcagtgat | catgccactg | tactcgagcc | tgggtgacag |
| 175501 | agtgagaccc | tgtctaaaaa | aaataaaaat | aaagataatt | gtgttttttt | taatgaagaa |
| 175561 | agagaggaaa | ctgggagctg | gttaggaggc | cttttcagca | gtttaggcaa | gcaagaagtg |
| 175621 | ttagtgaaaa | taacactatg | tcaacaagtc | acgtcactgg | aaacctcctc | ctctgcatct |
| 175681 | cattgacgtg | gagattccct | catgggttct | gtcctggagt | tgcaatgctt | caatctcaga |
| 175741 | aataagaatc | ctgaacaaaa | gcattctgca | aattaggtct | cacatagtta | gctatgaaat |
| 175801 | ttactttttt | atcctctttt | taagcctagt | tagactacaa | ggattttatg | caagcttttg |
| 175861 | aaattaaaaa | aaaaaaaaga | ggcaaatcac | atgagataaa | gacacatgat | aaggggtaag |
| 175921 | gcaagtggga | gcatcttgtt | gaagcaatga | aagataaata | cagctgagaa | gaaagaccat |
| 175981 | gatgtggcga | tttagacaca | ccaagaaatc | cttaacgctt | ccaataaaga | tatggatggt |
| 176041 | taagcatacg | aaatacacac | aaaaagaaaa | atattccatt | gaactgtatg | tcaatatatc |
| 176101 | atattttcag | cacccaaaat | ctagagagta | tggccaaagR | aaggatcttt | gtggcatttc |
| 176161 | tgtgcatgtg | aggcagtaca | ggaaatgtca | cttgcacatg | gaaatattgg | attttccagg |
| 176221 | aaatgttcgt | agttctctgt | agacaaagga | acaatattgt | ttaatttggg | accattcaag |
| 176281 | gaaattgagg | atatacagca | gacaacgaat | tacatgattc | tcttccttac | aaatagagtc |
| 176341 | taccatttca | tttcaccaga | tatcataatt | gtgggatgac | agaaggaatt | ttcatctcaa |
| 176401 | cttaatggat | aggaaaatga | agcacagaga | ggtgagaaga | atcactcttc | attcccggat |
| 176461 | gaccagctct | ggcctagacc | gccccatcca | accccacatc | caccattcaa | gctgctaggg |
| 176521 | cctgatcatc | taaccaatgt | ccataaccag | tcactgcatc | cttgtctttt | gccatcttgt |
| 176581 | tccacttaac | agaggaatga | gtttttctgg | caaggaaagt | ggaacagaag | gaaaaaaaac |
| 176641 | cctgtatttg | tcaagaaaat | aaaaaagttt | tcaagcaaca | tgtcacacat | acacttttta |
| 176701 | cacatgcaca | tttgctcatg | cacacatcta | catgtataca | ctcacatgca | cacatgctca |
| 176761 | cacacatgca | cattcataca | caaaccaatc | attcatccct | cagaagcagt | atatgtgtat |
| 176821 | atatatactt | tttaatttta | atttttgtgg | gtacttagta | ggtatacata | cttacagggt |
| 176881 | acatgagatg | ttttgacaca | ggcatgcaat | gtgtaataat | cacatcatgt | aaaatgggat |
| 176941 | atccattccc | tcaagcattt | attttttgtg | ttacaaacaa | tccaattata | ctcttagtta |
| 177001 | tttttaaatg | tacaattaaa | ttatacattt | aaattgtaca | tttaaaccca | ccccaccccY |
| 177061 | aacccacccc | acttctaacc | caccccactt | ccccttcact | ctcccactac | ccttccctgg |
| 177121 | ccctgctaac | catccttcca | ttctctgtct | ccgtgagttc | agttgttttg | atttttagat |
| 177181 | tctacaaata | agtgagaaca | tgcaatgttt | gtctttctgt | gcctggctta | tttcacttag |
| 177241 | cataatgaca | tccaggtcca | tccatattgc | tgcacatgat | aggatctcat | tggttttata |
| 177301 | gctgaatggt | acaccattgt | gtagaagtac | cacattttct | ttatccattc | atctgcttgt |
| 177361 | ggatacgtag | gttgagaaac | agtatatttt | tgtcatatta | cttctggcca | tctgagcaag |
| 177421 | tactgtccat | gtagtttcag | taaggaatca | agtaagttaa | caagtagaaa | atgaaaccca |
| 177481 | ggaaaaaaga | ttaaaaggca | ctgaaatgct | taattctgaa | ggacagaggc | tgaaaaataa |
| 177541 | tttcataaca | atctacacac | gtaacattta | cacagaggtt | gatgactgat | tgccaccatt |
| 177601 | gtcatataat | tgaatgtcta | ctccacacct | agtacaagta | tgctgatctg | gctctgctag |
| 177661 | gaacactaga | ggatgaattt | aaactttatt | atttgaattt | ctggctagtt | atctatatga |
| 177721 | aatctggaga | ataaaataat | tgttgagcac | tagaatggtt | tatgccaaat | gagagcttca |
| 177781 | gaaagtaaac | tctattcctg | aggtccattt | cagcaccatt | ctctatccca | ctcttttctt |
| 177841 | tcctctatct | tcgctgtatt | gttccatgca | cactgccctt | tttttttttt | ttttggtttc |
| 177901 | atgccctaac | ctatttcaag | gcataaccta | agtcccacca | tattgaaatt | aaataatttt |
| 177961 | cacctttgaa | aaaccccttc | aataaagcag | aattttggaa | tttatagatc | taacatccat |
| 178021 | actttcaact | gtttgcaaga | gatttcaatt | atttttactt | ttgttgggca | tgaattagat |
| 178081 | cccatgacat | gcaggaggaa | atcacttcct | ggtgagagaa | actaggccac | tgttgaacat |
| 178141 | ctacagttta | atgaggattt | gttgttctct | acgcatcttt | gtgcatgcag | taacatctga |
| 178201 | acataaactt | tggaattttc | aagtttctta | gtttgtatct | ctggcaaatc | tcaaggatct |
| 178261 | actgtaccta | aagtctatct | tacaatataa | atcagagtct | agaaaggcta | taatctgaac |
| 178321 | actcttcctt | aggaattctg | gaatctcatg | gcatgcttct | ggttcttcca | tttacMtaga |
| 178381 | agaccatttc | ggacagtgag | aaatacacta | aaacttcatt | ttgacagttc | ctggttagtt |
| 178441 | aagccaaaaa | attgatggga | aaaacttgtt | ttggggagtt | tagggtcagt | acacaataat |
| 178501 | aggtccaata | actctaccaa | ttctacaaga | tgaagaaaac | accatgagag | tgccctcata |
| 178561 | ctaaaggatg | aagaaggatc | ccagctctga | accctgtgag | agacagtggg | aaagagttcc |
| 178621 | atattcagtc | atggagaaat | gtcagagaag | gagaggtgat | catgcaggag | aagtgggatc |
| 178681 | atgtcataga | aatttccaga | gttctgctct | atggaaatgt | agtctaagtc | ctttaaaaat |
| 178741 | gatttcttaa | cataaaaaat | aagattacta | ttcagaggcc | aaaaaaattg | ttaccattat |
| 178801 | tggcgaggta | gaagtttcta | atctatatgg | tgagcagtgg | gaggtgacat | tctttggatt |
| 178861 | agaataaaac | tgactaattt | tgcaaaacta | ttggattgac | taaaagacaa | agtaacaagc |
| 178921 | ataaacgtaa | tagtgataac | actcatccct | gattgagttc | tctttatgaa | tcagcactat |
| 178981 | acacattatc | tcctttaatc | ctcctaatta | ctgtttgagg | taggtacctt | taagccgttt |
| 179041 | tacagagcag | aaagccaaga | cacaggaatt | taaggaaatt | tacaatcaaa | attacacagg |
| 179101 | gtgcaagtag | tagagttaga | attcaagcct | acatttgtgt | gatttcaaac | cttgtattct |
| 179161 | taaaattact | atgtcctatc | tcttccagaa | aatgacacaa | aggcaaggga | agactcttgt |
| 179221 | gttaaagaaa | aaatcccatg | taaatctctc | atattagaaa | aaactaaaaa | ctggccacct |
| 179281 | gatgtcaggg | gtggggtgga | gttgagacat | aagagtgaat | cctcaggggt | gactttagtt |
| 179341 | agaatggaat | ccagcctcga | attcccaaat | gatagggagg | gccaggaaac | ctaaagtttc |
| 179401 | tcttctattt | ttctctaatt | atttaatgtg | tggcaggtga | tctacgtgtg | gtaggttgta |
| 179461 | agttcttact | ttgtgggcaa | gtgttcagtc | tataataata | atatttttaa | aagaatattc |
| 179521 | aaccttcaat | tgatagattc | tcattcctgt | aagacaagac | attatggcct | tgcagaagca |
| 179581 | gaagtcagaa | tactgaccac | agttagtgtc | aaagggcttt | tcagttccca | aagaacttga |
| 179641 | gttttagtta | catcctcata | tgtactacac | ttgaccttag | ttatctaaaa | ggatgagaag |
| 179701 | cgactgtatc | ctcatatgta | aaaggcatat | tcaggaagag | actcttaaat | tccttggaca |
| 179761 | acaaatgttt | tctcctttga | atagctatga | ccttcaccct | tacttttaat | tgtaaacttt |
| 179821 | tagagcaaac | aagacataat | caactcatga | tccaaacttg | caaactatgc | atatgtagat |
| 179881 | ttatgtaaat | agccatgcaa | atcaggtacc | accactgggc | ttctgggaga | ttgcaaatgg |
| 179941 | agcccttggt | acagtagagg | tcaaatatcc | ttggcttaga | gctcaattcc | acatgcagat |
| 180001 | ttttacagga | caatggcagc | ccctaaacgc | aacagtcttt | aaatgctgtg | gtaattccat |
| 180061 | ggttgaatag | ttttccgtca | taattaggaa | ggggtgacct | ggttcagtga | aaaatgaaga |
| 180121 | gcagaatgaa | gttgtgctct | aatattccag | tttatttaag | tttgatactt | attatcatcc |
| 180181 | cttctcctgc | cttcctcatt | ttttcactgt | ccattaaggg | acctcagctg | gcatggaaca |
| 180241 | ttgctttcct | atttcagctg | cctttgactg | ggatattgaa | tttccctaac | ttacctaaaa |
| 180301 | gatgggacag | cccaaggcca | ctatccttga | aaatgcctct | tgccttgaag | cgtgggtacc |
| 180361 | ttccctgaga | tgctgcacca | atgatgccaa | ttaaattatc | aaaaactaca | ggaacaagag |
| 180421 | aatcttcagt | gactattacc | accactatta | ctaagagcca | cgaactctta | actccgaata |
| 180481 | atcaacaaag | accagaggat | ctctccaaaa | aggccattag | actttggaat | atccttgcag |
| 180541 | gaccttgctg | gacactcttg | ggaccagact | attactagtt | tctctgtcct | atggcagagg |
| 180601 | aacgggactg | tggagtccgt | gggggtgatc | ctaccaaatg | ttagacttgg | agagataaca |
| 180661 | gaagccagaa | atgtcctcgg | ccatccatgt | gtgtgcattg | gctattccct | tcacagccag |
| 180721 | ggttttgtcc | tcttctactg | cagagttcaa | ttgtagaagg | tagaagttcc | catgagattt |
| 180781 | ggtgagtcgt | gggagatggc | attctcagaa | ttgggatata | attgactaat | tctgcaaaag |
| 180841 | tactgaatgg | actaagaaat | aaggtaacaa | ccataaaggt | aatagtgata | atactcatcc |
| 180901 | ttgattaatc | tgtctttatg | aattagcact | atacacctta | cctcatttaa | tccttctaac |
| 180961 | tgcggcttca | ggtaggtgcc | attaagccat | tttacagagc | aagaaatcga | ggtacaggga |
| 181021 | atttatgcaa | cttactaatc | aagaattaca | tgggttacaa | gtggtaagag | tcacaggtca |
| 181081 | agcctacatt | tgtgtgattt | caaagcttgc | tttttttttt | tttttttttt | ttttttgaga |
| 181141 | cggagtctca | ctctgtcgcc | caggctggag | tgcagttgcg | cgatctcggg | gctcactgca |
| 181201 | agctcctcct | cccgggttca | cgccattctc | ctgcctcagc | ctccggagta | gctgggacta |
| 181261 | caggcacccg | ccaccatgcc | agctaatttt | ttgtattttt | agtagagaca | gggtttcacc |
| 181321 | gtgttagcca | ggatggtctc | gatctcatga | cctcaagtga | tccaccgcct | cggcctccca |
| 181381 | aagtgctggg | attacaggcg | tgagccactg | tgcccggcct | gtttgttttt | gttttcagga |
| 181441 | caaggtcaca | ctctgtcgcc | caggctggag | tgcagtgtca | ccttgaactc | ttggactcaa |
| 181501 | gagatcctcc | tgcctcaacc | tgagtagtag | cagggtctac | aggtgtgagt | caccaagccc |
| 181561 | agcttaaaag | cttgtatttt | tttttttttt | ttgaaacgga | gtctcgctct | gctgctcagg |
| 181621 | ctggagtgca | gtggcgcgat | ctcggctcac | tgcaagctcc | gccttccggt | ttcacgccat |
| 181681 | tctcctgcct | cagcctcccg | agcagctgag | actacaggcg | cctgccacca | tgcccggcta |
| 181741 | attttttgta | tttttagtag | agacggggtt | tcaccgtatt | agccaggatg | gtctcgaact |
| 181801 | cctgacctcg | tgatccgccc | gcctcggcct | ctcaaagtgc | tgagattact | ggcgtgagcc |
| 181861 | actgcgccag | gccaaaagct | tgtattttta | atcattattc | tctatgtctt | ccagacaatg |
| 181921 | atccaaagta | gtcatttcga | gtagtcggac | ttcgaacttc | accaatactc | cctgcccacc |
| 181981 | aacaaatctc | atgcagggaa | atttgaacag | ttcctaatgc | tggccactcc | aaatctactg |
| 182041 | ccacacagcc | ctgcctcctt | ttccccaagc | actttgtcct | acagcttgac | acagcaagaa |
| 182101 | tattcctgtt | cccttaacgc | atttccagga | ccgtggccaa | tggcagacac | caggtcatta |
| 182161 | aacccagtca | cagctgaatg | actaaagaga | ggacatgata | gctttaatta | atttttgcaa |
| 182221 | ttgagccctg | tgtttcttga | ataagattat | tacttctgag | ttcctctcaa | ttccatatcg |
| 182281 | aaaaaactca | gatcaaaatg | ccaatttcct | tccatgaaat | ggtctggaaa | ataacagaga |
| 182341 | taaaaatctg | aaagtattta | caaatatcga | ttgttaagtg | gagaaaaaga | aatgagtgag |
| 182401 | tttggaatta | gccaaagtcc | gtgaggagtt | tctgaaaaaa | cgtaatctac | caggagaaaa |
| 182461 | gctgtggttc | tagtgatgac | taaattatga | aaataaaatt | aaaagggggt | tgagatacgg |
| 182521 | atggggtagg | gatgtaaaaa | gtagaaattg | aagtttgctt | tgtttagtac | agagagataa |
| 182581 | ggatttgggg | aagaggggga | tgggataggg | gacattttct | ttaaatgaga | caagaaaaag |
| 182641 | taaacaagtt | atgtcatgta | cagaaaggag | aaatgaacca | acatccagct | ttcagtttgc |
| 182701 | tggctaacta | tttaggttga | tgcaaaagta | attgtggttt | ttgccattaa | ttttgatggc |
| 182761 | aattactttt | gcaccaacct | aatactttca | cttagactat | aaattctcac | ctgtggttag |
| 182821 | gtataaacca | tgtaagcact | accaatattt | gaaagagaaa | attgttctcg | cctgtaatta |
| 182881 | aaaggtatta | tattctcagt | cagctgcgtt | gtccaagaat | ccattaatca | aagaagccat |
| 182941 | tacatctcta | cttagattaa | ttccataatt | gtatcacgtg | agttatgttc | aaacatttat |
| 183001 | tgcccaataa | ttttcagaga | aaaatgtaat | aacttgggtg | gagatRtcat | taatgggaag |
| 183061 | agatttttgg | caagcaagtt | aatgaaatgc | atgaactaac | cctccaaccc | caattataat |
| 183121 | gaacttttcc | aagttccagg | gctaatgggt | aagctacaaa | cgctgtaaat | gagcttgcta |
| 183181 | gatttgggca | gccctctgtg | aatgtcaatt | tagtcctaga | attttgcaag | gataatttca |
| 183241 | agtgaacaaa | acctggacat | cttcctcatc | ttccaaaaca | ttcaaggcag | ttcttgtatg |
| 183301 | cccattaatt | ttttagcaac | aacttccatt | cgatctctgg | ataaaaggcc | atctttatat |
| 183361 | gaaaaaaatt | acttttcaaa | acaggcagta | ggtagttcct | ttttaaaatc | tcatgttgta |
| 183421 | aaacaatatc | attccaaaaa | taattctaac | aagactttat | aggctctcgt | aagagaattt |
| 183481 | attatgtatt | gccttttatc | accgtgcgtg | tcaaacagca | gaattatgag | ataaatggta |
| 183541 | atggtagtca | tgtgatattt | gctttaggaa | atctaaacaa | ttgtaaaaag | acactgtact |
| 183601 | tctatcaaat | acctaatact | tggttccaga | cttgaaatgc | cagaggttta | ctgcaagtct |
| 183661 | gagacagaaa | agaaggatta | aaaatgataa | aattaggatt | tgggcacagt | caatcataac |
| 183721 | atcttaacat | ctgacgtgac | ctctggagtt | atctgcacca | gacaagctct | cccagtcaga |
| 183781 | gatgaggtga | ggcccacagc | tgaggaatcc | aattgcccca | gcttcagaga | tgagtggcat |
| 183841 | Stgtaggaga | aaatccgaca | ggtccacagg | gaggggtttt | tattctttag | caacagaata |
| 183901 | ccagcagaac | acgttttatg | taaaatagcg | gatgaaatcc | caacctataa | ttgacacaga |
| 183961 | atggaaatgc | ttgggtagaa | ggtggagaag | aggccctcat | ccacctggca | ctgctctcca |
| 184021 | gctccctgca | atttctctgt | ggggagttat | cagacactta | ggtcttgggg | aaaatgcatg |
| 184081 | agttaggact | tctctggttg | caaagtaaac | aattcgatct | gagtgagaat | tacatccagg |
| 184141 | aaaataatga | tggttattct | aataatggtc | tcatggaccc | agaggagcaa | tgcatgtgga |
| 184201 | tttgaggaag | ggactacaat | tgggatggtg | gttatcagat | ctcgctctct | ctatttatat |
| 184261 | atatatttga | gacagagtct | ctgtcaccca | ggctagaatg | cagtggcggt | tcactgcaac |
| 184321 | ctccacctcc | tgggttcaag | tgattctccc | acctcagcct | cccgagtagc | tgggattaca |
| 184381 | aatatgcacc | accacactca | gctaattttt | gtacttttag | tagaaacagg | gtttcaccat |
| 184441 | gttggccagg | ctggtctcca | attcttgacc | tcaagtgatc | cacccacctc | agcctcccaa |
| 184501 | agtgctgaga | ttagacccat | gagccaccgc | acctagccct | ccctcccttt | aaacacacct |
| 184561 | gtgcacttct | aaattccata | ggacaagaat | ctgatgggcc | catgttgggt | agggcccatc |
| 184621 | aattacattg | ggggcttgga | agtggcagtg | tagagagaag | agggaggccc | agggtcatat |
| 184681 | gacagacaca | tggctggcta | tgatttaagt | gtcagtttcc | agagaagaag | aactggtcag |
| 184741 | gcccccagca | atttggaaac | tactgatact | gtgtctggaa | ttggtgggtt | ctcggtctca |
| 184801 | ctgacttcaa | gaatgaagcc | gcggaccctc | gcggtgaatg | ttacagctct | taagatggcg |
| 184861 | cgtctggagt | tgttcgttct | tccaggtggg | cttgtggtct | cgctggcttc | aggagtgaag |
| 184921 | ctgcagacct | tcacggtgag | cgttacagct | cataaaagca | gtgtagaccc | aaagagtgag |
| 184981 | cagtagcaag | atttattaca | aagagcaaaa | gaacaaagct | tccacagtgc | agaaggggac |
| 185041 | cccagcgcgt | tgccactgct | ggctcgggca | ccctgctttt | attcccttat | ctggccccac |
| 185101 | ccacatcctg | ctgattggta | gagcctagtg | gtctgttttg | acagggtgct | gattggtgcg |
| 185161 | tttacaatcc | ctgagctaga | cacaaaggtt | ctccacgtcc | ccaccagatt | agctagatac |
| 185221 | agagtgtgac | acaaaggttc | tctaaggccc | caccagagta | gctagataca | gagtgtcgat |
| 185281 | tggtgcattc | acaaaccctg | agctagacac | aggctRctga | ttggtgtgtt | tacaaacctt |
| 185341 | gagctagata | cagagtgccc | attggtgtat | ttacagtccc | tgagctagac | ataaaggttc |
| 185401 | tccacgttcc | caccagactc | aggagcccag | ctggctttac | ccggtggatc | ccacaccggg |
| 185461 | gctgcaggtg | gagctgcctg | ccagtccagc | gccgtccccc | ggcactcctc | agcccttggg |
| 185521 | tggtcaatgg | gactgggcgc | cctggagcag | ggggtggtgc | tcatcgggga | ggctcgggcc |
| 185581 | gcacaggagY | ccatggggga | ggggggaggc | tcaggcgtgg | ctggctgcag | gtcccgaacc |
| 185641 | ctgccctgcg | gggaggcagc | taaggcccag | cgagaaatcg | agagcagcac | cggtgggccg |
| 185701 | gcactgctgg | gggacccagc | acaccctctg | cagccgctgg | cccgggtgct | aagcccctca |
| 185761 | ttgcccaggg | tcggcagggc | cggctgctcc | gagtgcgggg | cccaccaagc | ccacacccac |
| 185821 | ccggaactcg | tcctggcccg | caagcaccac | gcgcagcccc | ggttgccgcc | ggcgcctctc |
| 185881 | cctccacacc | tccccgcaag | ctgagggaga | cggctccggc | cttggccagc | ccagaaaggg |
| 185941 | gctcccacag | tgcagtggtc | ggctgaaggg | ctcctcaagt | gccgccaaag | tgggagccca |
| 186001 | ggcagaggag | aagccaagag | cgaaagaggg | ctgtgagaac | taccagcaca | ctgtcacctc |
| 186061 | tcaatacgct | gtgctggcag | tggccacaca | tacatactag | aacgagacag | gcccaactga |
| 186121 | gctgcaggaa | gcatagctca | ccaagctggg | catctgagca | tggtacagag | caccatgact |
| 186181 | cccagtggtc | agttcaggac | aggcactggt | tttaccatct | ggggagccca | caaggatcag |
| 186241 | ggtagtatcc | aagactccag | acagcatggg | aaagcagaga | gtcagtgata | tggtttggct |
| 186301 | gtgtccccca | cccaaatctc | acatcgaatt | gtaataatct | ccctgtgtca | agggaggagc |
| 186361 | caggtggaga | taattgaatc | ctgggggcag | cttagccaag | gtgcagagtc | tgaggtctag |
| 186421 | ttctgctgac | aggaagagca | ggcctccatc | tggaaagaac | tggaaaccag | gagagtggcc |
| 186481 | tctaaataaa | cagaaattga | aggagacaga | agccttattc | ttatcctaat | tgagctctgg |
| 186541 | ggtcagggga | aagaagcagc | aagattattg | ggacagtgaa | tcccatgagc | taccaaccta |
| 186601 | ggccttccca | acctctgacc | agagcaagat | tgccttctgc | tttctgggtg | tgagtgctag |
| 186661 | gggagcctgc | aggaggaggg | gaatttccta | tgaggttgtg | gcgtctacta | gaaggccaca |
| 186721 | atgatcaaaa | gggtcattag | agcttctgct | tgctctgaat | ggtatttgtg | gagagggaat |
| 186781 | gtgccttatg | ttctctcttg | cacagtggtg | gtgtctccct | tgacccttcc | taacaaacac |
| 186841 | ggtgagtgaa | ccatgaaacg | agaaaactat | caggcatcag | taatacgtaa | ggcctgggcc |
| 186901 | aaaataaaga | gttatgtgtt | gaaaatgaac | taacgcaact | ggtagatact | ttccatgcta |
| 186961 | acacttttgt | agtctcaggg | tttataaaaa | tctttctttc | tttctttctc | tgtctttctt |
| 187021 | tctttctttc | tttctttctt | tctttctttc | tttctttctt | tgtttctttc | ccttccttct |
| 187081 | ttccttcttt | ccttctttct | ttccttcttt | ctctctttct | cccttcctcc | cttcctccct |
| 187141 | tcttcccttt | ctttcttcct | ttctttcttt | cagcctgggt | cttattctgt | cacccaggct |
| 187201 | gtagtgcagt | gtcataatca | tggctcactg | caacctggaa | ctcctgggct | caagggatcc |
| 187261 | tctcatctca | gtctcctgag | tagctagcac | tacaggcacg | tgccaccaca | ttgggctaat |
| 187321 | gttttaattt | ttttgtagaa | ctggggtctc | actatattgc | ccagactgtt | ctggaactct |
| 187381 | tggcctcagg | tgatcctccc | acctcggctt | cccacaatac | tgggattaca | gacgtgagcc |
| 187441 | accaagcctg | gccagtttac | aaagatttct | aaagaaagat | cccaggaaac | aatttcaagc |
| 187501 | atcgctccta | catgtcactt | tgtgatattt | ttcatgcata | gaaggaataa | tacattaagc |
| 187561 | ctgcgtgcat | ctcagtgcgg | tacatgtttt | actcatttgc | ttaatttgct | gaagatttta |
| 187621 | atgtcacgtg | gctgcagaga | gggacatgga | aacacttaga | aggactcagg | gctttaacat |
| 187681 | caaacgccca | cttttgattc | tcctgccaag | aaagaacaaa | atatttgtct | ctcggatagt |
| 187741 | taacctctct | tccatacaca | gtccacaaca | cattgcattg | gcaagctata | tgcaaccccc |
| 187801 | ttctcctcta | taaaggatgg | taatcttttt | gcttacaaag | atgagaaaca | ccaggaacac |
| 187861 | ccatcattct | ccaacaaatt | agcttgtctt | gagactattt | tccatgtaat | ttaagggtaa |
| 187921 | atgtcagaga | agtgtttccc | tttattggaa | gtaaggagct | aaagttctaa | atgctggtaa |
| 187981 | gaaatgcagt | ttgtaactcc | ttcaggcacg | gggtctttat | ctgccctgaa | tgatcttcta |
| 188041 | gtgcccagga | ttcagtgtcc | agggaaggaa | tagacacctg | tctgtgtaac | ccaagggtga |
| 188101 | cagagcttag | catctttttt | ttttcaattt | ctttcttaca | tccaaagact | ctggatatct |
| 188161 | tacatataaa | ccaacccaag | ggttttctga | catattttgc | ccaagttgga | gttatacaca |
| 188221 | tgtgccaatc | accagtgaag | ggtgattact | tattcagtag | aaaggagctt | tgaagaacgg |
| 188281 | tgggaagtag | ttctgtataa | atgacacatt | ttatctgttt | ttcaaataat | tcatggaaaa |
| 188341 | tactgacata | aagtgccaat | tattttttcc | ccatgaatag | ggctggcagt | cttattgtat |
| 188401 | actttaaaag | tcacttacaa | tttgctcaca | tttcttaact | ccccagggaa | aagaatgtat |
| 188461 | ttcctttgct | ctaaagatta | aagttcaatt | tgacttcata | ttttgtggaa | gggattctta |
| 188521 | aaggcattgc | attctcatat | cctagatatc | attacaagct | gtaaatttgg | tgattgatgt |
| 188581 | ctacatggca | gcctccagag | gctgcgtgct | agttaaaata | ataaagtgct | ccttagcctc |
| 188641 | tagatgcatt | cgagagaata | gcaggtggtc | attgtttact | gtgctaaagt | gagccactcc |
| 188701 | gtttgagaca | gctcattttg | gactcaagct | gaatgccatg | cctgagtgat | ctgacttcta |
| 188761 | acaaagccac | caatagactc | attctgttgt | ttaatttgaa | tgagaattag | ctgctgcttc |
| 188821 | gtgcaagcca | aagaccctct | attagaaact | ggttctctag | aaatgtttat | ggaagcccag |
| 188881 | tggctgcagc | attttggagg | ggtgtgagaa | tgatctgatc | ttccatggaa | gcacattgca |
| 188941 | tttccaatct | caccattcca | cttcagactc | aagcagatgt | ggcagggaga | ggcggtaata |
| 189001 | tattttgcca | tatgtcaggg | ccactgtgtc | actcagactc | ccaggaaatg | acacagactc |
| 189061 | agggaaaaca | gtggcccata | tattttcatt | aaaaagagag | catgtgactg | gttcacagtc |
| 189121 | agggcaattg | acacagaccc | cttctccaag | agcgttatat | taatattagc | aagagcacag |
| 189181 | ggccacggct | gctgaagttt | tagtccacag | agtcaagaaa | gcacaaaatc | caaaaagctg |
| 189241 | ggatttgcaa | gacactgttc | ccaaaagcag | tctccctgat | aggtttgcac | aaaactcttt |
| 189301 | ggggtaagca | gtaactgtac | ccttcacact | gtggcaggtc | cagccacagg | caacatagaa |
| 189361 | cattctagcc | ttgcatctta | aaacatgcct | tttaataatg | tgacctggag | aatctgagtt |
| 189421 | caacatacag | agtgtctgaa | cactttagaa | gtgatttatc | agggtcctag | tataagaatc |
| 189481 | gaggcccagg | cagaaccaaa | ctgcctgctt | ctaacaactc | cttttcgaag | gaccagcttg |
| 189541 | tttttatata | tttctttgtg | atattgctcg | gaatctgtgt | ctccattcag | aaacccatcc |
| 189601 | agtatctatt | ttttttcaaa | aagaagacag | accatttgtc | taagatcaga | tgagcgtgat |
| 189661 | ggaggacaca | aacattatga | ttgaaatata | atcaagatat | taatgcaatc | ggtactgttt |
| 189721 | ttagaagaaa | aaaattgatg | ccataattca | gggtcaccat | ggcaatcctt | atttgcttat |
| 189781 | taatttgtta | ctactttaaa | gttttcctct | tggctcattc | aatgtagcct | ggcaaacatc |
| 189841 | cataccttgt | ttctcatggt | gtttatttgc | tcattccatg | atttttacca | atgatgctgg |
| 189901 | gcattttcaa | gattcagcct | aacaatgtac | atggacaggc | ttagtctcct | ctgggatttt |
| 189961 | ctgcctggga | tgatgcaatt | tccaggagct | cattacacca | taaaggagac | gcacgcccct |
| 190021 | tttcttagga | aacaatcttc | tgtctgttgg | aggtctcttg | ctctccagtt | taaaaagctt |
| 190081 | taactgtgct | ttaagacttt | gtcattcaat | tcttatttat | tcttcttaat | ctccttggga |
| 190141 | ttcttacatt | aggtgtacaa | taatcctgtt | aagccctcaa | gacttctcct | aaatgagtgg |
| 190201 | cagttctggg | taatggattg | aaatatccct | tgcattttct | agaaacactg | tatcttgttt |
| 190261 | tatgatcgtc | gttttctggg | agtcccagct | aatttaggaa | gtggggaaaa | gaaaacattt |
| 190321 | gctgaatctc | agctgagaac | aacacgaaga | tgatcatcct | agggaaagta | caaagtatac |
| 190381 | aaaaataata | caaaaaggca | Sgtttctctc | cttcaaaaag | aaagtcccca | tttattcttt |
| 190441 | cccagtgaag | tctctgactt | ctgaactctc | tgactactcc | caggcagttt | gatgtagttc |
| 190501 | tcttttcatt | gttgtggata | caatggtgaa | ccttggataa | tggtgtgcct | cacagaccag |
| 190561 | gcaggaagtg | agaacctgaa | atctgcagaa | gcagggtttt | tagaaagcgg | tagaatctta |
| 190621 | acaggcgaga | agtcaaaagt | cagaatgcaa | gggtatcaag | gtgaagtaag | acagaggttc |
| 190681 | agcaaccagg | aaagaacaca | atgccacaag | ttcccaggtc | aaagaaagac | agagggtcag |
| 190741 | caagctgttg | ggcatctagg | actctactca | tgagacatgt | ggaacagagc | ttgtaattca |
| 190801 | gcctctgtga | aaaagccctt | gacatgattt | ctgctgtgga | aggagtcatt | ttcattgtat |
| 190861 | ttgcagccca | gggactgaaa | ataagggctg | atgatgatgg | gaatataata | gaatgctaag |
| 190921 | ctgggtgtgg | tggctagcgc | ctgtaatccc | agcattttgg | gaggctgagg | taggtggatc |
| 190981 | acctgaggtc | aggagtttga | gagcagcctg | gccaacatgg | taaaactccg | tctctattaa |
| 191041 | aaatacaaaa | ttagccaggt | gtggtggtgg | gtgcctgtaa | tcccagctac | ttgggaggct |
| 191101 | aaagcaggaa | aatcgcttga | acccgggagg | cagagttcgt | agtgagctga | gattgcacca |
| 191161 | ttgtactcca | gcatgggcaa | ccagagtgaa | actctgtctc | aaaaaaaaaa | aaaaaaaaaa |
| 191221 | aagaatgcta | aaagggctct | attgaaaaag | tatatttctt | acaacctcca | ctacttaata |
| 191281 | tagaccccct | tcctgtattt | gttcattttt | taaaattccc | agaatagatt | actgcaaagt |
| 191341 | ttccaacttt | cagagatctt | taaggccatc | tggaagcttt | ggatataaga | catatctaat |
| 191401 | cttaaagcac | atcacccaca | ttagcacatg | aagttctgct | gaggaattta | ctcagtcctt |
| 191461 | ctgagcttca | aggaacaggt | taattccaat | gcctttgtct | gaaaaaaaat | ggatccttcc |
| 191521 | tttctagagc | tttccctggc | aatgaccaat | atcacaagaa | cagagccctt | ctcagacagc |
| 191581 | attgcatcag | cacgagcata | gggccatggc | tgtccaggga | gtcaagaaaa | cacaaaatat |
| 191641 | aaaaaacctg | agatttgcaa | gacattattc | ccaaaagtag | cctcctggat | tcttttgcat |
| 191701 | aagactgttt | gggatgggca | gtaagtgcag | ccaggacact | tgcaagtcca | gccatgagca |
| 191761 | acatagaaca | ttctagcctt | gtaccctaga | acatgcattt | ttataatgtg | atctggagaa |
| 191821 | tatgagttca | atacacaggg | tcggaaagtg | ggagctaaga | caggaaggag | gatagggtgg |
| 191881 | aactcaaaaa | caagacttag | gtggtgatgg | agaatgctaa | aattttagcc | atataagtac |
| 191941 | agaaaaaaat | agacaataac | acatgtatgt | atctatttca | acagtttctg | ggtgtttaca |
| 192001 | gtgtaccgag | cactgtatat | ttgaccagat | accaaagaga | gtcggtgaga | aaaataccag |
| 192061 | tggcctctaa | ccatctctgc | attcagacag | caagaaagtt | ttgagagaaa | ccaggatatc |
| 192121 | taggaaacga | tccagacagg | tttggaaaga | tagtcctttt | agacacagca | cttcaggaag |
| 192181 | tatatgttaa | aatggcaaaa | gctctagatt | gaagtctggg | tttcagtttc | ggttttagaa |
| 192241 | ttttgagcaa | gtctctaaat | tttactggct | ctctgtttct | acttccatag | acacgatgct |
| 192301 | ttgttaaccc | tggaggaagg | acatcattgt | cctgcaggat | gctgggtgtt | gttatgaatg |
| 192361 | caagagccag | caggggatgg | tacagaggat | gcagagacca | actctcccta | agagctcaaa |
| 192421 | gcagcttcac | aagcatttga | actgggcctt | gaacagtgag | cacaaattct | tcaggcaaac |
| 192481 | agagatggta | agttgtctat | ttctggcagt | gaatagagaa | aatagcattt | attaaggtcc |
| 192541 | tgaagaattc | atgctttcat | ccatttattc | aacaaatatt | tagtggtaat | ttattaataa |
| 192601 | tgaacaaatt | attagttatt | attagttatg | ttgattatta | gtattattaa | ctgacactgt |
| 192661 | tgtaggcact | ggactacagc | aataaataag | gcacaccatg | tatctgtcct | caaactctac |
| 192721 | aatccagtgg | cagggaatga | aggaaactga | ggtcaagtta | tgcaaagtga | accagagatg |
| 192781 | atgcacaaaa | ggatgatgct | ccggcagggc | atggtggctc | acgcctgtaa | tcccagcact |
| 192841 | ttgggaggcc | gaggcaggtg | gatcataagg | tcaggagttc | gagaccagcc | tggccaacat |
| 192901 | agtgaaaccc | catctctact | aaaaatacaa | aaaaaaaaaa | aaaaaattag | ctgggcatgg |
| 192961 | tagcgggctc | ctgtaatccc | agctactcag | gaggttgagg | caggagaatc | gcttgaacct |
| 193021 | gggagacgga | ggctgcagtg | agccaagatc | acgccactgc | actcctgcct | gggcgacaga |
| 193081 | gagagactcc | atctcaaaaa | acaaaacaaa | acaaaaaaat | gatgatgccc | catggatggc |
| 193141 | tcgaaccaga | aataggtaaa | gatgagatcc | aaagtggtac | tgaagacatt | tcagatttat |
| 193201 | taactacaca | caaagagtag | ttggggcttt | tgaggttttc | tgtttatcca | ttcagaaata |
| 193261 | tttactgaac | acttgatgtt | tctgacactg | ttctaaaatc | tttgcatgca | tatttccgtg |
| 193321 | gcaatcttgc | aaggtaagaa | ctattccatt | atcattttat | aatacatacc | aggaaatgag |
| 193381 | cactgaaagg | catgagtact | atgcctacgg | ttatatgata | ataaggagta | tatcttggat |
| 193441 | ttgaacccag | ctcgggctaa | ctccaaagcc | ctttctcatt | ttactacatc | agtgaatcac |
| 193501 | accaggctga | tcatagcaga | atttattgag | ggaggggccc | atgtatcagt | attaattttt |
| 193561 | ctatgattta | tttatttatt | ttaatacaga | tggggtctca | ctatgttgtc | caggctggtc |
| 193621 | atgaactcca | agcctcaagc | aactctcctg | cctaggcctc | ccagagtgct | gagattacaa |
| 193681 | gcatgagcta | ctacatccag | cttcagtatt | tattttattt | tattttattg | tagtttagtt |
| 193741 | ttttttagtt | tagtttagtt | tagttttgag | ataggatatg | gctctgtcac | ccagactgga |
| 193801 | gtgcagtggc | gtaatctcgg | ctcactgcaa | cttctgcctc | ccggtttcaa | gtgatcctcc |
| 193861 | cacctcagcc | tcctgagtag | ctgggactac | aggcacatac | caccgcacct | ggctaatttt |
| 193921 | tgtatttttt | gtagatacag | ggtttctcca | tgttacccag | gctggtcttg | aactcctggg |
| 193981 | ctcaagcaac | ccttctgccc | cagagtccca | aagtgctgga | attacaggtg | taagccactg |
| 194041 | cgcctggctc | aactttactg | tttttaaaaa | gatgtctagc | tagttccagt | gtgcacctag |
| 194101 | gctagggaat | agttgcccta | tataaaaagc | aaaacacaaa | caaaacaaaa | ccctcaggat |
| 194161 | aactacctac | tgatttaaaa | tataagttgg | cattcattag | catcttgctt | attttactca |
| 194221 | tgtaatgatt | tgcttggatg | cccaggttga | tgggaaaaag | gggttccatg | tagttgaaaa |
| 194281 | acctagacag | tagtcaacat | gcaggcacca | tgctcgttcc | tcccctgaca | ttcaacgcta |
| 194341 | ggttgagatc | acatcgttac | atgggataac | accgtctttt | attgttcttt | tattgctatg |
| 194401 | tgatctcctg | aactgtgttc | ctccaactta | gaatttaaaa | agaaacattt | ctgggttctg |
| 194461 | cctagtaaaa | cataaatctt | ggaaaatcaa | atcccaacta | gagaaatctg | gtttggatca |
| 194521 | gagtaactaa | atcattaatc | tggatctttc | tccaaaactg | gaacccaccc | aaatccaatc |
| 194581 | catgatatat | atgaaagagg | ccagatattt | agatggacat | taattctttc | tttttgttga |
| 194641 | tgtgtttgtg | ctgacagaag | agtcagagac | caaaggcaga | atactgaaag | attgtgtttg |
| 194701 | ggtattttta | gcctgattaa | aataaggaat | gatttaaaac | tatttcaaat | agctcttccc |
| 194761 | acagaattct | gcaaatcttc | cagcctgctt | tctggaccag | agaagcttgg | ctaaaggagc |
| 194821 | tggacatcgg | gcaacccctc | ccatttggat | tgagcctcca | acattcagag | gttgcacccc |
| 194881 | atcagtctaa | acaacatttt | tttYtgcgtg | atagaaacac | agaccacaga | ttggaattgt |
| 194941 | ctgtttgtac | ttagatggga | agtcctagca | gttctcatca | acccttgaag | acgttaaaaa |
| 195001 | tcacgccaca | gttggccaac | cgtctgttta | cctcttgaag | aactctctca | gatgacagta |
| 195061 | atcacaggca | ggttctgaat | cacaggctga | tgttaacatg | taccaaggct | ataccccctg |
| 195121 | gaaaagccta | tttccagcct | tctgaggcta | cttcattact | accctggctt | tcctggctcc |
| 195181 | taatggtcag | ggtggaggga | gggacggagg | taggggagat | ctgggaagcc | tggttagcta |
| 195241 | gaagttcctg | ccttgtcaca | gcactaatcc | tgcaatttct | ccattggatt | tctggactga |
| 195301 | tgagcaattt | tgatgctgat | aaggcagaaa | cacaatcact | ctcctgggcc | cttgtatttt |
| 195361 | gcagggtaac | ttggataata | caccccatct | cggcacctct | gcgagtgtaa | ctcatttcct |
| 195421 | ctgcccagag | gagaaacaag | gactccttgg | acaaaacggt | gcttcctttc | atagccaatg |
| 195481 | aattgggcat | ttctccaggt | tctgtgaagc | ttatttagtg | acattcccca | aaacctgtgc |
| 195541 | cccgagaagt | tccttcttct | ctgactttag | gtagagtccg | agattcattt | cagtgatttg |
| 195601 | gctaacatgt | aaagtgtctc | acatgtgaaa | gagaatcaaa | ataacttgag | cacaacaaca |
| 195661 | gacaaacaaa | agcaatactt | gaccaagacc | tcttgtactt | tttctgctct | gggactctcc |
| 195721 | aagcactgtt | cacaaatgca | gtgtatgtgg | aaatctatgg | tattgttatg | atctgtcagt |
| 195781 | gatggtgcat | attaaaaccc | agttttagtt | aatattcact | catagccttt | gcttgttaat |
| 195841 | atttacacac | ttggcttaga | agatagaaag | gctcggccag | gtgtggtggc | tgacgcctgt |
| 195901 | aatcccagca | ctctggaagg | ccgaggcagg | cggatcacga | ggtcaggaga | tcgagaccac |
| 195961 | cctggctaac | acagtgaaac | cccatctcta | ctaaaaatac | cataaaaaat | tagccgggag |
| 196021 | tggtggcggg | tgcctgtagt | cccagctact | caggaggctg | aggcaggaga | atggtgtaaa |
| 196081 | cctgggaggc | ggagcttgca | gtgagctgag | atcgcgccac | tgcactccag | cctgggcgac |
| 196141 | agagcgagac | tccatctcaa | aaaaaaaaaa | aaaaaaaaaa | aaaagaagat | agaaaggctc |
| 196201 | aacagaattc | tacagaaaga | aactcgaacc | atccaatcat | gacgttagcg | atgtgttgtc |
| 196261 | tctaatcaag | ttacctgatt | acctaggtaa | caggaggcct | Yagcagtcac | ccctggcacg |
| 196321 | catggaccac | ttactggcaa | ctcttttgtg | gctgtgaatt | catatagtat | ttggttaaat |
| 196381 | ggtgacacaa | aattgatccc | tagtgcgata | ttcagtggat | ttttctcatt | tctaaatggg |
| 196441 | cattccacac | tgaaaacttg | catgccagtt | gcccatacag | aatgctcttt | tattctttaa |
| 196501 | gaatggaaaa | taagtcattt | tcatttgatt | cagagtggac | aacctcacgt | ggggacagga |
| 196561 | aagtctaacc | agagatttca | actggggtgt | ctgaagtgta | acttgctaga | tacatttgga |
| 196621 | actatcagac | attctatctg | ctatacttag | ggtagaggga | ttaagcctgc | atattcaaga |
| 196681 | atgaaactcc | agcttttaag | cctagcttat | tattttatgt | atgtgggcaa | attagttagc |
| 196741 | ctctcaaaac | cttagttact | tcattttaaa | aataattata | ctgtaatccc | agcactttgg |
| 196801 | gaggctgagg | tgggtggatc | acctgaggtc | aggagtttga | gaccagcctg | gccaacatgg |
| 196861 | tgaaacctca | tctctactaa | aaatacaaaa | attagccggg | tgtggtggca | tacgcctgta |
| 196921 | gtcccagcta | cttgggaggc | tgaggcacga | gaattgattg | aacctgggag | gtggaggttg |
| 196981 | cagtgagctg | agatgaagcc | attgcacttc | ggcctgagtg | acagagtgaa | actctgtctc |
| 197041 | aaaaaaaaat | aaaaataaaa | ataattataa | aatagaatcc | gcagtgaggc | ttaggcaaga |
| 197101 | tcaactctgt | caagtggctc | gcattctgtg | cattgcacaa | gtggtaaatt | cgattcaata |
| 197161 | aactatctat | tgttatcatt | ttattattac | taaatgtgct | taatcttgag | aggctgctgt |
| 197221 | aaaactcagt | ggcatttctR | gtcttaaagc | tggtttcaaa | aggaaacttc | tccattcgca |
| 197281 | ttatgacatt | taaagttgtt | tctctgaggc | tttgggaaga | tctccaagtt | ctcctaacac |
| 197341 | aatagagatc | atttttatac | tccctgtaac | acctacgtgg | ttagaggaca | agaactgggg |
| 197401 | atctttatta | Ktccagagtc | tgtccaagtg | atgtcaagga | tcctgagttg | ggacaagaaa |
| 197461 | gtagggttac | atgccccaca | tcctactgct | acattttggg | cttcggggct | cttggtctgc |
| 197521 | tcagggtttg | cccgggctgc | aaagtagtgc | tggctcatga | ctctaataac | tgcattaacc |
| 197581 | atgtcctcct | ccttccatct | caaggttgcc | cagtgggaat | tgtgaatgcc | gggactctgc |
| 197641 | atttcacagt | tagtggatct | ggctaaatag | atggagtaag | tgggcttaat | tgatttttcc |
| 197701 | ccaagttgag | acttggggta | aagaggcaag | aatgggtttg | tagtgaagaa | tcaacttacc |
| 197761 | actgtcaaca | tgcgataaga | aatctaaaag | attttcattg | ttcactaaac | aagacttcag |
| 197821 | ctgggactcc | ctctaaaaag | caacaaactg | tgattagagg | agtcccaaag | gatgatacga |
| 197881 | gctaagataa | atgagctaag | acatgttcta | tttgaaacat | ttgggtcttt | cttggcaaga |
| 197941 | gacatactct | tatcttgata | gtgtctcctt | tccttttcaa | tttttattta | ttataaacaa |
| 198001 | agattatcct | cagacttgaa | tctgttttgc | caggatactc | ccctccaaca | ttttgtcctt |
| 198061 | ttccactgct | tcccttctct | tttttttttt | ttaaactata | ggtatgtaat | aggaatttcc |
| 198121 | ttccttcaag | taattgattt | atttgtgctg | acatcatcaa | aatgtctcaa | catgaatttg |
| 198181 | atttcatgtc | taagatgttt | aagaatacat | atttcttacc | cttaaaaaca | gaaagtcatt |
| 198241 | ttttttttta | gtaaagtaac | tcacccctga | aaaacttggg | ctctgttcaa | aattcccaac |
| 198301 | atgtgacttt | gaggatcatc | taaaagagtc | tctcctggtc | tttcaccata | acgttcccac |
| 198361 | ggtaaattca | ctaaaaacag | aaatgctcaa | tatataagtt | aaaaaccctg | tcaatttgac |
| 198421 | ccacattcga | aatgtttcag | tgtatccaga | acacatctgg | accaaggtgg | gttgtttttt |
| 198481 | cctccttaaa | aaaaaaacga | aaaaaaaaac | cctcactcgt | ttatgtcttt | atatcttaaa |
| 198541 | caaaatgaat | taRtggcttt | taaacccttg | aagcactaaa | aaaggaaagt | gctttcagtc |
| 198601 | tgacaacttc | aatgataagt | tgcagtccaa | tgtgttttga | aatggaccaa | acataaatcc |
| 198661 | atcttcaata | gggagtttta | attggaagcc | acactgaccc | ttaactccag | tgttgctgcc |
| 198721 | acacagcttt | tgataatgaa | gactgacaaa | ttgtcttcac | tggatgtaga | ctttcaatgc |
| 198781 | actcctccca | cggccttggc | cgcaccaggc | attgggaagg | agacgtgcat | ttcccaggtg |
| 198841 | tgagaggaca | acagaggtcc | actccaagct | actggacagc | atccccctcc | atgggggaag |
| 198901 | caggtgcctg | aggaggaaag | ggtgctggga | gagaatcaca | catatctgag | cagtgggaag |
| 198961 | cccagggaaa | cacctctgaa | gttaaaaaga | aggaggatgt | caggggagag | agaaagaaaa |
| 199021 | gaaaccatct | ttcttatgga | gaattccatc | tgcttgtggc | ctgtttggga | actaagttat |
| 199081 | agaatgtttt | tgggcttcct | aagattgatg | tgggaggggt | tgcagatctc | atgcagagct |
| 199141 | ctgatgattt | tttttttttt | ctgaagaaca | gcaaaccaga | aaacaacggg | agtttgtagc |
| 199201 | ttagagagtg | ctgttgagca | gagattgtga | tattgagacc | attccaggag | aggaagtttt |
| 199261 | aaggccaaaa | ttaaaataRc | acatttgttc | cttgcaattt | gacttcagaa | tcccccaagg |
| 199321 | ctccttcctt | gagaagcctt | attttttatc | tgagagatgt | tgtgcaccat | atggattttt |
| 199381 | tttttttttt | ggtttacgtt | ctgagtttgc | caagaatgtc | ttatgagaca | cagtacctca |
| 199441 | ttttccatgg | gtgtcccaag | agatatcagg | taccttcaaa | gctctctgta | tttgctctgg |
| 199501 | taaatgtaaa | catgcagaat | aaaacaacac | tatttagcaa | ataaaatgat | actctcataa |
| 199561 | cttttaagct | gtgcaaagca | taattactgc | aatctcaata | attattatca | cttaatgttg |
| 199621 | aaagagcacc | aaccttcatt | cagaagcaaa | ttaaaattgg | aacccagcct | tgacattcat |
| 199681 | ctattaagac | tcaaagcctc | aacagaaggc | atatacatgt | ttgaacattt | tgactgaatt |
| 199741 | Yacaggtgaa | ttcaatcaat | gtgtcagttc | cYactgaact | ttcaagttga | ttgactacat |
| 199801 | tgattgaata | agttgaccta | ttgtgttgtc | agcttggaga | cttagccatt | gcatcatcat |
| 199861 | ggaagtaggg | agttagctat | gggactcaga | cctcagtgag | aaaggagaaa | tctggagtag |
| 199921 | ctgagtatca | gtctctttgg | aattcttcac | atttgttact | ggtatgatgt | tggcaaccct |
| 199981 | caaaagaatg | aatccattaa | agtggacttc | cagtggagtt | gtagaagtgt | tgaatttaag |
| 200041 | gggaaaacat | tcaaccctcc | actgacaagc | tcctttagac | ttctttggct | tcactttttt |
| 200101 | attgctggca | gtcttggaat | aacagactgc | ctcctgatct | ttcagcatct | tctctgtagt |
| 200161 | tctcaggctt | actgggtact | gtgatttcaa | tctggaagac | tctccattgc | ctggatcttt |
| 200221 | gttagctaag | aaaccacctt | cttttcattt | cctaaacagt | tttaagattg | agaacaatta |
| 200281 | ttcttgctgt | ctgattttat | ctgctgaatc | ataaaaatct | tatccagggc | tcaaaataca |
| 200341 | tacacagtga | cagaacagag | agaaggaaca | gaactcccag | ccctttgaag | atcttgtatt |
| 200401 | ttgttcatgt | aggggtattg | tttgtttgtt | tttgaagttt | gtgcttggag | tactctagtc |
| 200461 | tacatgtttc | ttgaggaata | ttccaggaaa | tatcaaatac | atacgtatac | atatatatat |
| 200521 | gcaaaaaact | tttaaatatg | acttatgtaa | ataaaaagtg | taaattgtct | tataatataa |
| 200581 | aaatattcac | agcaagttgc | taaagtaccc | ccccccaaaa | aaaacctatt | gtatttagca |
| 200641 | caataactaa | aagaagtaca | actgcccctt | gaacaatgca | tgggttggaa | taccaccccc |
| 200701 | caactcagtc | aaaattttgt | gtgtaactgt | ttgttttttt | tctgagatgg | agtcttgctc |
| 200761 | tgtttccgag | gctggagtga | gctggtgcaa | ccttggctca | ctgcaacctc | cacctcccag |
| 200821 | gttSaagaaa | ttctcctgcc | tcagtctccc | aagtaattgg | gattacggta | tgtgccacca |
| 200881 | caaccagcta | agttttttgt | atttttagca | gagacagggt | ttcaccatgt | tggccaagct |
| 200941 | ggtcttgaac | tcctgacctc | aagtgaactg | ccccccctgg | gcctcttaaa | gtgctgggat |
| 201001 | tacagatgtg | Rgctaccatg | tccagccaaa | aatttgtgtg | taacttttga | ctcccacaaa |
| 201061 | atataactaa | tagccaactg | ttattggtaa | tagctttacc | aataacataa | acagttaaat |
| 201121 | aacatgtatt | ttgtatgtta | tatgtaatct | atactgtatt | cttacaataa | aatgaactag |
| 201181 | atgaaagaga | ataacattaa | gaaaatcata | aggaagagaa | aacatattca | caatttgtta |
| 201241 | aatgaaggcc | tttatcctca | gtgtcttcat | gttgagtagg | ctgaggagga | ggaagaggag |
| 201301 | gggttggtct | tgctgtctca | ggggtggcag | aggtagaaga | aaatcgatgt | ataagtggat |
| 201361 | ccacgtgtaa | gtggacccac | gtagttcaaa | tctgtgttgt | tgaaggatca | actgtaaaga |
| 201421 | gaacagggaa | cacaaccaca | acaacagctt | agccaaactg | aacatgccgc | cttgaaaagt |
| 201481 | cttgcagcaa | gctgtctccc | ccaatattct | acagtaacta | cctttgtcca | ttccgtttct |
| 201541 | gtttctaatt | tcttggcttt | tgtaattact | agtttcatta | cgctcagaaa | ggatggtcag |
| 201601 | gtagacagac | acctactaaa | agtaatgccc | caaactaaag | gggctaataa | atggtgaagt |
| 201661 | tctagaaggt | aatcattgtc | acttgttgag | catgaactgt | gctccagaca | ccagagcaag |
| 201721 | tgttgtatat | attttaactc | aattaatcct | caccatagag | catggtttgg | ggcgtcagac |
| 201781 | agagcttgca | tccatgtctt | agctttgcca | tttaactgct | tgcacgaatg | tagaaaagta |
| 201841 | acttaaatct | ctccagtctc | tgtttgttca | actgtcatca | ggtactgaag | agcatctgac |
| 201901 | aatttcctaa | ggtaactcta | caagagggaa | tgatgcatgt | tgaatttctg | catatgacat |
| 201961 | tataccatgt | atgaattctg | tttgccacca | gggcttacct | tcacctgcac | agtcatcttt |
| 202021 | ctaccacccc | aacatgccca | atttctttag | tcttttttgc | atggatctta | tttccaaggc |
| 202081 | attgaatcat | cttcactgtc | ctttctgaat | tatgctcgaa | ctctccataa | ttcattttag |
| 202141 | ttaaactcca | agcaaaacac | agctccctaa | taagggcctg | tctgacatcg | agtttaagag |
| 202201 | agggattagt | gtgtgcattt | tgcatgccat | atacctatac | tttaatccct | aacccatcgc |
| 202261 | tatgaaatag | cttgccctct | atttcSaatg | gagaagctga | ggcatgaaga | ttaatggtgt |
| 202321 | atactgacat | cacacaaatc | cagcaacaat | ttagcattct | ttgctctgat | ccaattttca |
| 202381 | ttaaacacca | atactctgtc | tctacttttt | ttcttgaaga | aacaagagca | acatgttcct |
| 202441 | tggaagatta | gaaggagaaa | gatgttggct | ttttgttttg | taagtacata | aatggtgttt |
| 202501 | gaatttttta | tataggtaag | gctagatcag | ccttgagcag | tttgctgtaa | gtgcaatgat |
| 202561 | tagaaccaca | ttgaagcaaa | acgaaagtac | aaatagtgtt | ttgtatgtta | aactattctg |
| 202621 | ttcaagtttt | tggtgaggac | tgggttttct | ggttcacttg | agacttttaa | acgactatta |
| 202681 | tcaaaataac | ttaaaacaga | atcaaaggaa | attaattcct | ttaccatgtt | attgaagacc |
| 202741 | atttcggttg | aaaaagaagg | acaacaaatg | aatagggcaa | aggattctga | gagaatccct |
| 202801 | gggtgtgcaa | ttcctgaatg | atagactggg | tctatcttct | gcattctgat | attgtctgtg |
| 202861 | ctgctgcatt | aatgtccatt | tatgtagtaa | atacttagta | aaaccctatg | cccacattag |
| 202921 | agtttatttt | ggaaggagga | agatgaaaac | aaaaggtatt | tcacttcatc | tacatctagg |
| 202981 | ttatcagcag | aggtcttggt | cttttcctgg | tcttataaac | attctataag | accattcctg |
| 203041 | gttttataaa | cattcttata | aacatataag | tatgaagaag | atattcccca | aaagagagtg |
| 203101 | atttcaccga | gacttcaaag | aaaagctcta | cacttggaca | tactttcttc | tgtgtctctg |
| 203161 | tcagtgtctt | attaatattg | ttcaccatca | tccaggtctc | agatagaggc | ttggcttttc |
| 203221 | cctttggaaa | tctggcttct | ggactttttt | tatggcattg | ctgtctggaa | ggctgagttc |
| 203281 | tgatggtgtt | ttctctatgt | gagtgaaagc | agcagtttct | tattttagaa | gaagcatttt |
| 203341 | tagggtttca | tcacatctgt | caaagacaaa | gccctagagg | tgaaatcctt | cctctacaag |
| 203401 | caagctcata | tctcctcatc | tctggtatct | caaacttccc | ttggaagata | ataaatggca |
| 203461 | ggtccttgag | aaacaggctt | gtcctcagag | aagcccaaag | tgctttaagg | gtcagtcaaa |
| 203521 | aggctgaaag | ttcagcattc | aactcctctg | agcactttgc | ccttgtgtcc | agccttgcag |
| 203581 | ccctggtgac | agttaacagc | cggattcttg | gtgacgtgga | aacttgatca | ccacttcagt |
| 203641 | gcacgggcca | tcaactatgt | gaccctgggg | tgaaagcctc | catgttatgt | tacctctccc |
| 203701 | cctcagccag | gccatccccc | aaaagcactg | cttttcaatt | tatatttata | ttgtctttcc |
| 203761 | ctccttcctg | ttctttcaca | cgaggcagct | gacaatggta | ataaatgtgg | tcgtactcaa |
| 203821 | ggatggggct | gtgacaggct | caataRggag | gggactgatS | tgttgctttg | gggttgaaag |
| 203881 | ctcagacttt | catgaaacaa | aaatattata | ttcagggtaa | tgatttatgg | ccttgactcc |
| 203941 | tgtttgattg | cagaccataa | atactagcaa | acggtactta | cggcatctct | agaggacatt |
| 204001 | aggaacatgc | tcaagtttat | tttatacagc | aattactgat | ggactataag | catggacatt |
| 204061 | atttgaaaga | cattattttc | tccaaaataa | gtctttgtag | atgataatgt | tcaacaatga |
| 204121 | ttttcataca | ctccaaccca | atccatgtga | ctcgaaagtg | ctcaaagaaa | tccaatgaca |
| 204181 | gatcaactag | ttgatgccaa | tcRatactct | gacaaggatc | tcatcttttt | tgttttttat |
| 204241 | cctgtaataa | gcttctcagc | ctcagatgca | tggcctatga | ctagcattat | cccttcaaaa |
| 204301 | tttagaaacc | acgaaatcat | ctggttgtaa | acttactgcc | tgggattgtc | ataggaacct |
| 204361 | tgttcaaatc | tctttgattt | cctcgtgtct | aatttgttta | gctgtaaagc | agggatacct |
| 204421 | tctccagggg | atagtatggg | ccttaatcac | atcatttcta | ttttcctgcc | ttttctgatg |
| 204481 | tagtctcatc | attactctca | ctctagagta | gatcctgagt | tctcttaccg | ccttgactat |
| 204541 | tccaaagtct | tgaagccatg | agaatgtgat | actattatcc | cattataact | gaagccggta |
| 204601 | ccagccagaa | tgtagaggag | tgacttatta | taagaatgtt | tcatttatat | tacaaaaaaa |
| 204661 | atttaaatcc | caagaggaat | ttatttatat | tccctgtatt | tagtgctcag | tttattaatt |
| 204721 | tatttaaagc | actaaattac | atttttYtaa | aacagatttg | atccttatgt | ggaaaagaaa |
| 204781 | aagacctctc | taaaactttc | tattgagtgg | taggagttgg | atccaaaaga | tgctatttga |
| 204841 | aatgctattg | aaggaaagtc | tcgctgcaga | taggagaatg | aatggcttag | gggctacttt |
| 204901 | acattcatta | ttacaattgg | tagccacttt | tccctcctta | tcttatttga | gagacttccc |
| 204961 | agaagataga | aaccaattaa | ctctttattt | tggcagtgat | catcctaggt | gttcttttaa |
| 205021 | cgggtattat | acttgctaaa | ttatagatga | gcttgctagt | aagacaaatg | ccattatctg |
| 205081 | agttatttat | tcatgggaca | tcgtctgcaa | catacccaca | ctagtttttc | gcaaagtttg |
| 205141 | gtattgatca | attgctttaa | tcagttgtac | caagggatgc | taaaagactc | agctatcaca |
| 205201 | gctttgaata | tcagcatctc | tctctgggtc | tctgccaggc | tgggagtgaa | aactgcagct |
| 205261 | attttcaaac | tcttaataac | aagaacgcta | agtagaagta | aatggctctt | tccatttaga |
| 205321 | tgggtgaatg | ttcatcatta | ctccgccttc | atacccatga | actcatcact | ttgacagtat |
| 205381 | catcatattt | ttgtttctgc | attttcttcc | tttggaatgt | tagctggaat | gtttgcaaac |
| 205441 | atctagcctc | ctggcctctt | ttaatgatac | ccacatatct | tttcagattt | tgctttcaaa |
| 205501 | gcagggaaca | aactctgctg | gttcctcaga | aacacatggc | caagatcctc | agtagataat |
| 205561 | cagcattctg | tctagaggca | attaaagatg | atatagttgg | ggctgtattt | ttatttatac |
| 205621 | tgtgtaatga | ggctccaaat | taaaaaagtt | atatcctaat | ctccattaat | gtaaggagtt |
| 205681 | gcagtcttgt | gagtgcagtg | gtccatatta | gatggcaaat | ttgtcatttt | tacactttcg |
| 205741 | ttcagtgcca | agattccgca | tttcctgagg | acaagtgctg | atgcaaatag | caggagctct |
| 205801 | acccacctct | cttttcgtcc | tactgtggaa | gtgggtttca | tttgagcaat | aagacatgac |
| 205861 | agacagcgca | tttgcgggca | attatgtcac | accttgggtg | gtatcagaaa | gcataaggtc |
| 205921 | aaaggatggg | ggctttaact | tcctaagaaa | tgcaataatg | accctgaaat | gcacagcttg |
| 205981 | ggtaccttat | tacaattgta | ggatggaata | cattaaaatg | aagaaaacaa | ggaacaaaga |
| 206041 | tgaaagtggg | acttggtcga | tgggggtatg | aaatcaatgt | caggcaatca | taagtctttg |
| 206101 | tatttgaatt | tctaaaagca | tttccaaatg | gttatccctt | cataaagctt | tagtactttc |
| 206161 | tcatgcacaa | tcagacacgg | attgtctaca | aagagcttct | aaccagttcc | tctacctttt |
| 206221 | tttaatgaag | ttggtaagaa | ttggaagaag | aaccatttct | ttttgtttga | gaagaaaact |
| 206281 | ttgtcaaggc | tggtggctgg | catctgcacc | agttttagac | aggccagWgg | taaggatggg |
| 206341 | acagtaccag | ttggagagga | tcagaaccct | gtggggtaag | taagcaccat | tgaatcactg |
| 206401 | ctgagaaatt | caaggcactc | tgtataaaga | caagacctgt | ggtaggctga | aaaatgcacc |
| 206461 | cctcacaaag | acatctgtat | cctagtccct | ggaacctatg | aatgttacct | tatatgacaa |
| 206521 | ttaaaaaaaa | taaataaagg | gtctttgcgg | attgattaag | taatgatctt | gagaaaggga |
| 206581 | gattgcactc | aatttccata | tgagccttaa | ataccatcat | atttccttgt | aagagagagc |
| 206641 | cagaggctgg | gtgcagtgac | tcagccctgt | aatcccagca | ctttgggagg | tggaggcaga |
| 206701 | agggttactt | gggcctagga | gttagagacc | agtctgggca | acatggcaaa | atcccatctc |
| 206761 | tacaaaaaat | acaaaaatta | gccagatgtg | gtggcatgcg | cctgttgcct | actcgggagg |
| 206821 | cagaggttgc | agtgagctga | gatcaggcca | ctgcactcta | gcctaggcga | cagagagaga |
| 206881 | Ytccatcttt | aaaaaaaaaa | aggaggcaga | ggaagattct | acacacagac | acacagagaa |
| 206941 | ggtgatgtga | ccacggaggc | agagactaga | cagatgcccc | cacaagccaa | ggaatgccag |
| 207001 | ccatctcgag | aagtgtgaag | aggcaaggaa | cagattctcc | cttagagctc | agtagggtgc |
| 207061 | atagccctgc | tgataccttg | gtttcaacac | agtgaaactg | ctttttggac | ttctggcctc |
| 207121 | cagaactatg | agagagaaag | tatttgttat | tttaagccac | ctagtttgtg | gtaatgtgtt |
| 207181 | gcagtgacca | ccggaaagta | acgagtccca | gtgggcttgg | ggctcccggt | gtctcaggcc |
| 207241 | ccacccctct | gggattgttc | ttctaggcta | ccatgtgggt | gtatttctac | atcatgcact |
| 207301 | gttttcaaag | aacccaaaag | tagacgatgt | cttgactacc | gttcagctgc | tttctcccYt |
| 207361 | gctatcattt | tctgtgggaa | ctatgagaca | ggaattaagc | ctcacagaca | cttcttttac |
| 207421 | aacYtcatgg | ttttcctcac | cctcatcctc | ccctgaccca | gagaagcaaa | atcctacttc |
| 207481 | tttttccaag | cctaattctc | tcagagaggc | ttctccttga | gcctctgagt | ttgcctccct |
| 207541 | cctcatagtt | catctcgtac | aaactatgac | tatcaatttt | aacccttctg | tgccatttgc |
| 207601 | cactgagaca | ccttatgtca | cagccatctc | cctggtgagg | ctctaacctc | taccaaggac |
| 207661 | agaaccttgt | cacctttcta | tcttccaagg | tgccaggacc | acatttccca | tcttgagatg |
| 207721 | taatgtttct | ttgaatagtg | atattgcttg | atggaaggat | acaagagctg | tccaaggaag |
| 207781 | gtgggctggt | cctgtgtttc | tgcttcggcc | tggagtctcc | tacatggtgg | gaaagtaaga |
| 207841 | gcttagacag | ctcaataggc | ccaaactgca | agacgtcatg | ccccaatact | tcttacagat |
| 207901 | gccaagtaag | agacgctgag | agatcaagag | acaggcagaa | gccgcaggga | tggagcaaag |
| 207961 | taggggtacg | ttaaagagaa | tcctaactgc | tccaagcaga | gttgagaacc | catagtttgt |
| 208021 | gtaactcaga | cagaccactt | aaagaaatgg | ggtcaaaaag | ccatgcatga | atatacctca |
| 208081 | gagatactgc | aagtgcagtc | tcaacctgtc | ccaataaagc | gagtcacgtg | aattttttgg |
| 208141 | gtttcccagt | gtgtacgaaa | tgtatgctta | cactgtactg | tggtctatta | agtgtgcatt |
| 208201 | atcattatgt | ctagaaaaac | aatgtacata | ccttaaagaa | aaaaatgctt | tattgttaaa |
| 208261 | aatgctaata | ataatctgag | ccttcaacaa | gtaattattt | gctagtggag | ggtcttgcct |
| 208321 | cgatgttgat | ggctgttgac | tgatcagact | ggtcattgct | gaagtttgag | atggctgtgg |
| 208381 | caagttctta | aaataagaca | atggtgaagt | ttgccacatc | gattggctct | ttcttccatg |
| 208441 | aaagatttcg | ccgtagcatg | agaagctgtt | ggatagtatt | ttacccacag | cagaactttg |
| 208501 | tttaacatta | gagtcaatcc | tctcatattc | tgtctctgct | tctcaactta | ctttatggaa |
| 208561 | tattctaaag | cctttgatgt | agtttcacca | atgctcactg | cgtcttcacc | aggagtacat |
| 208621 | accatctcaa | gaaaccactt | tctttgctca | tccataagaa | acaacttgac | atctattcaa |
| 208681 | atttgattat | gaaattccag | caattcaatg | acatcttctt | gctgcatttg | taattctagc |
| 208741 | tctcttgata | ttcccaccac | atctgcagtc | acttcctcta | ctcaagtctt | gaacccctca |
| 208801 | aagtcatcca | tgagggttag | aatcaccttc | ttcaaaactc | ctattaatat | tgatattttg |
| 208861 | atttcctcct | gtgaatcatg | aatgttctta | atggcatcta | gaatggtgaa | tcttttccag |
| 208921 | aaggtttcca | atttattttg | cccggattca | tcagaggaat | cactctctat | ggcagctata |
| 208981 | gccttacaaa | atggatttct | taaatagtaa | gacttgaaag | tcaaaattac | tctttgattc |
| 209041 | atgatctaca | gaatggatgt | tgtgttaccc | agaatgaaaa | caacgctcat | ctccttgtac |
| 209101 | atctcttcca | gagctcttga | gtgatcaggt | gtattgtcaa | tgagcaataa | cattttgaaa |
| 209161 | ggaatatttt | ttcctgagca | gcaggtctca | acagtgggct | taagatattt | aggaaaccat |
| 209221 | gctgtacaca | gatgtggtat | cattcatgct | ttgttgcttc | atttctagaa | cataggcaga |
| 209281 | gtcaatttag | cataatttta | agggtcttat | gattttcaga | atgatcaatg | agcactggct |
| 209341 | ttaactcaaa | gtcaccagct | tcattagccc | ctaacaagag | agtcagcctg | tcctttgaag |
| 209401 | ctttgaagcc | aggcactgac | ttttcatctY | gagccatgaa | agtcctcagt | ggcatcttcg |
| 209461 | aatagaaggc | tgttttgttt | acattgaaaa | tctgctgttt | agtgtagcca | ccttcatcaa |
| 209521 | tgatcttagc | tagatcttct | ggataacttg | ctgcagcttc | tccatcaaca | cttgctgctt |
| 209581 | tacttcacac | ttttaggtta | tggagatggc | ttctttcctt | aaacataaca | atccaacttc |
| 209641 | tgttagcttc | cagcttttct | tctgcagctt | cctgacctct | ctcagccttc | atggaattga |
| 209701 | aggagttgag | acattgctct | ggattaggct | ttggtttaca | ggaatgttgt | gactggtttg |
| 209761 | atcttctatc | cggacaaata | aaactttctc | catatcagca | ataagaccag | ttcactttct |
| 209821 | tatcctttgt | gggttcactg | gagtagcact | tttaattccc | ctcaataatt | ttcctttgcc |
| 209881 | ctcacaactt | ggctgtttgt | cccaagaggc | ctagctttca | gcctatctca | gctttccata |
| 209941 | tgccttcctc | accaagccga | atgatttctg | cttttgactt | caagtgagag | atgtgttact |
| 210001 | cttcctttca | cttgaacact | tagagaccac | tggaaggtta | ttatttggcc | taatttcagt |
| 210061 | attgttgtgt | cttctgaaat | agactctagg | agaggaagag | ggacagggaa | tggccagtta |
| 210121 | gtgaagcaat | cagaacaaac | agaacattta | tcaattaagt | gcaccatctt | agatgggcac |
| 210181 | agtttgtgga | accccaaaac | aattacaata | gtaactgaca | tcaaagatca | ttaatcacag |
| 210241 | agcaccataa | ccgatacaat | aataatgaaa | aagtttaaaa | tattgggaga | attgcaaaaa |
| 210301 | tgtgattcag | agatatgaag | tgagcacctg | ctgttggaaa | aatggtgccg | atcaatgtgc |
| 210361 | tgctcagtgc | agagttgtca | caaaccctca | attcgtaatt | ttttaatgca | acatttatga |
| 210421 | agcactataa | tgcaaagtac | gataaaacga | ggtacgcccg | tatctcagtg | aacacctagt |
| 210481 | gtgctgtgtt | gcatggatgg | gtggtcacat | gtccctgtct | ccttccacat | acatccactc |
| 210541 | atagtgaacc | cagaatactt | ctgggaccag | atgtgtgagg | atttttcccc | acacactaag |
| 210601 | caattcttca | gtggacacca | actaggtgtc | ctataattta | attcaatgtg | acactatcta |
| 210661 | cWtggaaatg | gtgtcagacc | ccacaggtta | agggctcagt | ctcacaatgc | ttccccaact |
| 210721 | ttagacacca | attgcaaggt | tagtacattg | tcgcctgtgc | ttctgactaa | ccgtctataa |
| 210781 | atcgggggtc | ctactactcc | ctccatgggt | ttaatttgct | agcatggctc | acagcactca |
| 210841 | gggaaacaca | cttacttatg | ttcaccagtt | tgttataaag | gatattacac | aggatacaga |
| 210901 | taaacagctg | aggaagagat | ggataccgtg | agttatgggg | gtgcacaact | tccaccccgt |
| 210961 | ctctggggct | ccaccctccc | agtacctccc | tgtattcagc | cactggaaac | tcaccaaatc |
| 211021 | ttgttgttca | agtttttgtg | aagcttgatt | tctatttgcc | ccccaccaac | ccaccagcta |
| 211081 | tcagtaggtg | gggctgacag | cKccaacctt | ctaatcctct | aatcaccttg | tctttctgtt |
| 211141 | gactggctcc | atacagagac | tatctagggg | Ygtcacccta | cgtaatctct | ttaccataaa |
| 211201 | ctcagaaagt | gatcaagtgg | cttcattata | aataacaaaa | atagtcctat | cactcagaag |
| 211261 | attccagggg | ttttaggaac | cccatgtcag | gaactgggaa | caaagaccaa | acatatttcg |
| 211321 | tataataccc | caatgaccat | taccaattgt | catagccatc | cRaggataca | gatgtatttc |
| 211381 | ataaatagtc | ctcttagtgt | ggcttaggga | taaaagtttg | tagacacctg | ctccaaactt |
| 211441 | tgaacattga | atttaaggtt | ggccaacctc | aggcactctg | gtgtgttatt | tataaataat |
| 211501 | aattcttcac | ccccctcgtc | ctcttatctg | tcatatgaag | agcccatggg | aacccttagc |
| 211561 | atcaggaatc | caactaattt | ccatttgatg | ggaggataca | gctggacttc | ctacccccat |
| 211621 | actgacattc | ctacttggca | gaggcgtgac | actaccctta | tttaggattc | aagaaacttg |
| 211681 | ctatttaaga | tttaggaaaa | attcctttct | tccagggaca | tttctcttga | acatgttttg |
| 211741 | tttcacatgt | aatttagtgg | tcacctaggg | gccaaagaaa | aacagagact | tttgtctatt |
| 211801 | tgtttatttc | ttaaggctat | catgaaagaa | aatccacgaa | taacttgagc | tgtcaatgga |
| 211861 | aacctttttt | taggactRga | tctctccttc | acatttaggt | agtgactggg | acatttatgg |
| 211921 | aagaaaaata | aataataata | ataattatta | tgatagctta | tcattgttca | gcctttgttc |
| 211981 | tgtgccaaag | atacagatga | tctcatctca | ttcttacaac | aaccatgtga | aattggtgct |
| 212041 | atgattaatt | tcagtttcac | agaaactgag | gccagtgagg | ttaagaaact | tcctaaaatc |
| 212101 | acacgcagct | agttagtgtt | gcaaaatctg | aacccaagca | atatgtctca | gtagcatgtg |
| 212161 | ccctctagta | tatatgtgta | tatatatgta | taggataaRa | ggattcatac | ttttgtagca |
| 212221 | ggtataaatc | ttagtctata | aactcagatg | ctttattttc | cataaatcaa | tgtaatatcc |
| 212281 | tctctaagcc | cttttcccac | tgggctggag | gaaataaaag | cttgaccatt | cttgaggttt |
| 212341 | ctctatcacc | atcttcagca | gatttgagca | gggcttgggg | atctgcctga | catgtcaaga |
| 212401 | tattggatag | actaggcYcc | tctgtttgat | gcctgttgag | aggcacattg | tccctggcca |
| 212461 | cgtgataatc | tctgttctgt | gggcttctgt | accaagcttg | ggaccaagag | tcttggggca |
| 212521 | cttccccagg | tgtatcacac | actcaccttc | cctgaggtcc | cgccatggcc | tcagggagca |
| 212581 | ggagcagatc | ctcatactct | tggactaggg | aatttttttt | tctttttttt | ttttttttga |
| 212641 | gacagtctca | ttctgttgcc | caggctggag | tgcagtggcg | caatctcgac | tcactgcaac |
| 212701 | ctccacctcc | caagttcaag | tgatcctcct | acctgagcct | ccagagtagc | tgagcctcca |
| 212761 | gagtagctgg | gagtacaggc | atgcgccacc | acgcctggct | aacttttgta | tttttaatag |
| 212821 | tgacggggat | tcaccatgtt | ggccaggcta | gtcttgaact | actgacctga | ggtgattcac |
| 212881 | ctgccttggc | ctcccaaatt | tctgggatta | caggcgtgag | ccactgtgtc | tggcctggac |
| 212941 | tagggaaatc | ttaattgctc | tgagaaaatg | caatctcctc | ttctttcctc | cctccctcct |
| 213001 | tctctgtctt | ttactctctc | ttctctcccc | gtgagagagc | tgcactttgc | actctttctt |
| 213061 | aagccaccga | tcttctcaat | ggactcagaa | ttttgacagc | ttttacagta | gggaacccta |
| 213121 | ccacagcacg | gaccttgata | cctctttgaa | caggcgaagg | tcaaggcaaa | acacagagtg |
| 213181 | aaagcttcaa | taaatatttc | aaaacacact | cttgccacat | cacctttcgc | ttatttttac |
| 213241 | aatatgctgc | acgcttactc | actctcacac | tcacaaacat | aactgcacaa | acatcatttc |
| 213301 | ccattctttt | gatgtttaaa | tactttcctt | ctcctatcag | atgaagttta | tattatctag |
| 213361 | gctttgtctc | tgacaattct | gatgttgttg | ttatggaaat | atgtcaagac | atttctttgt |
| 213421 | aaaaattccc | aatgaggctg | ggcacagtgg | ctcacacttg | taatcccacc | actttgggaa |
| 213481 | gctgaggtag | gaggatcact | tgagtccagg | aatcaagacc | agcctgggca | acattgtaag |
| 213541 | accccatctt | tacaaataat | agtaataata | attagctggg | catggagtgc | atgcctgtag |
| 213601 | tcccagctac | ccaggaggct | gaggtgggag | gatggcttga | gcttgggaag | ttgagggtgc |
| 213661 | agtgagctgt | gatcatgcca | ctgtgctcta | gcccaggtga | cacagtgaga | ttctatctaa |
| 213721 | aaaaaatttt | ccaataaact | ttacattata | gtgatgcccc | tgtggttgtg | ggcaatgtat |
| 213781 | aaatgatttg | actaattttt | tcccatatgt | caattcaacc | ctcaatattt | tccaaattta |
| 213841 | tcttagaatt | tatatttaaa | tttatgaatt | atcttgaaag | acctgattgt | ttggaatcta |
| 213901 | ccctcatcta | ttctacttat | ttgaatcttt | agaaagatta | ttaaaggaat | atttaagtgt |
| 213961 | gttgtttcat | ggaaattagg | aataccgagt | tataatgtaa | atatattcat | tcagaaactg |
| 214021 | gggtgggggt | cgggggggaa | gtcctcaggg | aagatacagt | attcactatt | gacgttggtt |
| 214081 | taatattgct | ttgtacagtt | gtttaatatg | tacgttgttt | gtattttatc | ttgtatatat |
| 214141 | ctctccacca | cacattaatc | cttggactgt | tagtacatac | agaacacatg | tcgggatgct |
| 214201 | ttctcagtgg | acctcatctt | cagggatcat | tttatttcca | cagtttagtg | cctccttact |
| 214261 | agaatagaga | actccataat | tttttctaaa | gattctatca | gtcttgatcc | aatgtgctag |
| 214321 | gtgataaatg | actgaacgct | ggccccggag | gggaagttag | caatgagtca | tgctgtctcc |
| 214381 | tagtaaccag | tcagtaaata | gcttccagca | aatattttaa | acctcaaagc | acacttacct |
| 214441 | ttggtattat | ccttagacag | actatcctca | tctatatctg | cactctcatc | tctctttcag |
| 214501 | aacttctaag | aaaggagaaa | aagtgggatc | gggtttgcat | atgacgtcac | ccccttgaaa |
| 214561 | acccacatga | aaacattcct | accccaggga | gtctgtctca | acaaaccttt | tccttgattt |
| 214621 | gtttctctgt | attttgcttg | ccctcaaatg | ttatgaagcc | atgtatactc | cacgtaattt |
| 214681 | tcatttgttt | gtttctagat | aggcccttct | atgtaactat | tcaatgagtc | catactgatg |
| 214741 | tgtgatttca | gagtttagca | tggaggtcag | cacacgatca | gaacagaaag | cgacactcaa |
| 214801 | gcaccctgct | cacacatccc | accaatgaca | ctgtttcaga | ctaattaatt | tacatgtgta |
| 214861 | aaggttattt | aaaacataaa | tcctccctaa | gtgagaggtg | ggaaagggga | gaattaagcc |
| 214921 | tattaatctt | gttcacctta | tccccacgta | agactgtttt | ctttagacat | ttagggtcag |
| 214981 | actctgtagc | atgggagtcc | ttattaggga | tatggaaaaa | aacacgagag | ttcgttttgg |
| 215041 | agcatggaac | atcaaaagat | gtgtctatgt | aaatgaatgc | aaaaggctac | tttagatgga |
| 215101 | cagagagcag | caagagagct | ccagacttta | gcaaaggagg | gatgcaaaga | gaaataacta |
| 215161 | ggaagggaag | atgagtagca | ggaaccaagg | tcactgtgaa | ccctcttagc | ctaactctaa |
| 215221 | ggtacggctg | caagtctctg | aattaaactg | tgagcctttt | aaagatgtga | tttcttctgt |
| 215281 | ttccttgttt | tcctctaaaa | ctctatacag | agtaagtgtg | cagcaaatgt | ttgctgaaca |
| 215341 | aatacacttc | cattttaggt | aaaaagtata | gatggtccct | agacagaaaa | taaagcacta |
| 215401 | agactgttYg | gcagggttat | aagaggtagc | gctgagattt | tgccaagaag | gcttttggct |
| 215461 | tttggctttt | ggaaaattag | tcccaaaagt | tagtgctctc | ttggataaac | tgaacccccg |
| 215521 | atccttcttc | agggcatctt | ttaaaattta | tttatttttt | atttttttca | gaccaagtct |
| 215581 | cactctgttg | cactcccagg | caggagtgca | atggggtgat | ctcggctcac | tgcaacctcc |
| 215641 | acctcccagg | ttcaagctat | tctcctgcct | cagcctccca | agaagctggg | attacagatg |
| 215701 | ctcgccacca | tgtctggcta | atttttgtat | ttttagtgga | gacagggttt | cactatgttg |
| 215761 | gccaggctgg | tctcgaacac | ctgtccttag | gtgatccacc | cacagggcat | cttttttttt |
| 215881 | tttatttttt | ttattatact | ctaagtttta | gggtacatgt | gcacattgtg | caggttagtt |
| 215941 | acatatgtat | acatgtgcca | tgctggtgcg | ctgcacccac | taatgtgtca | tctagcatta |
| 216001 | ggtatatctc | ccaatgctat | ccctcccccc | tcccccgacc | ccaccacagt | ccccagagtg |
| 216061 | tgatattccc | cttcctgtgt | ccatgtgatc | tcattgttca | attcccacct | atgagtgaga |
| 216121 | atatgcggtg | tttggttttt | tgttcttgcg | atagtttact | gagaatgatg | gtttccaatt |
| 216181 | tcatccatgt | ccctacaaag | gatatgaact | catcattttt | tatggctgca | tagtattcca |
| 216241 | tggtgtatat | gtgccacatt | ttcttaatcc | agtctatcat | tgttggacat | ttgggttggt |
| 216301 | tccaagtctt | tgctattgtg | aatagtgccg | caataaacat | acgtgtgcat | gtgtctttat |
| 216361 | agcagcatga | tttatagtcc | tttgggtata | tacccagtaa | tgggatggct | gggtcaaatg |
| 216421 | gtatttctag | ttctagatcc | ctgaggaatc | gccacactga | cttccacaat | ggttgaacta |
| 216481 | gttgacagtc | ccaccaacag | tgtaaaagtg | ttcctatttc | tccacatcct | ctccagcacc |
| 216541 | tgttgtttcc | tgacttttta | atgattgcca | tgctaactag | tgtgagatga | tatctcatag |
| 216601 | tggttttgat | ttgcatttct | ctgatggcca | gtgatgatga | gcatttcttc | atgtgttttt |
| 216661 | tggctgcata | aatgtcttct | tttgagaagt | gtctgttcac | gtccttcgcc | cactttttga |
| 216721 | tggggttgtt | tgtttttttc | ttgtaaattt | gtttgagttc | attgtagatt | ctggatatta |
| 216781 | gccctttgtc | agatgagtag | gttgcaaaaa | ttttctccca | tgttgtaggt | tgcctgttca |
| 216841 | ctctgatggt | agtttctttt | gctgtgcaga | agctctttag | tttaattaga | tcccatttgt |
| 216901 | caattttggc | ttttgttgcc | attgcttttg | gtgttttgga | catgaagtcc | ttgcccacgc |
| 216961 | ctatgtcctg | aatggtaatg | cctaggtttt | cttctagggt | ttttatggtt | ttaggtctaa |
| 217021 | cgtataaatc | tttaatccat | cttgaattga | ttttYgtata | aggtgtaagg | aagggatcca |
| 217081 | gtttcagctt | tctacatatg | gctagccagt | tttcccagca | ccatttatta | aatagggaat |
| 217141 | cctttcccca | ttgcttgttt | ttctcaggtt | tgtcaaagat | cagatagttg | tagatatgcg |
| 217201 | gcattatttc | tgagggctct | gttctgttcc | attgatctat | atctctgttt | tggtaccagt |
| 217261 | accatactgt | tttggttact | gtagccttgt | agtatagttt | gaagtcaggt | agtgtgatgc |
| 217321 | ctccagcttt | gttcttttgg | cttaggattg | acgtggcgat | gcgggctctt | ttttggttcc |
| 217381 | gtatgaactt | taaagtagtt | ttttccaatt | ctgtgaagaa | agtcattggt | agcttgatgg |
| 217441 | ggatggcatt | gaatctgtaa | attaccttgg | gcagtatggc | cattttcacg | atattgattc |
| 217501 | ttcctaccca | tgagcatgga | atgttcttcc | atttgtttgt | gtcctctttt | atttccttga |
| 217561 | gcagtggttt | gtagttctcc | ttgaagaggt | ccttcacatc | ccttgtaagt | tggattccta |
| 217621 | ggtattttat | tctctttgaa | gcaattgtga | atgggagttc | acccatgatt | tggctctctg |
| 217681 | tttgtctgtt | gttggtgtat | aagaatgctt | gtgatttttg | tacattgatt | ttgtatcctg |
| 217741 | agactttgct | gaagttgctt | atcagcttaa | ggagattttg | ggctgagacg | atggggtttt |
| 217801 | ctagataaac | agtcatgtcg | tctgcaaaca | gggacaattt | gacttcctct | tttcctaatt |
| 217861 | gaataccctt | tatttccttc | tcctgcctga | ttgccctggc | cagaacttcc | aacactatgt |
| 217921 | tgaataggag | tggtgagaga | gggcatccct | gtcttgtgcc | agttttcaaa | gggaatgctt |
| 217981 | ccagtttttg | cccattcagt | atgatattgg | ctgtgggttt | gtcatagata | gctcttatta |
| 218041 | ttttgaaata | cgtcccatca | atacctaatt | tattgagagt | ttttagcatg | aagggttgtt |
| 218101 | gaattttgtc | aaaggctttt | tctgcatcta | ttgagataat | catgtggttt | ttgtctttgg |
| 218161 | ctctgtttat | atgctggatt | acatttattg | atttgcatat | attgaaccag | ccttgcatcc |
| 218221 | cagggatgaa | gcccacttga | tcatggtgga | taagcttttt | gatgtgctgc | tggattcggt |
| 218281 | ttgccagtat | tttattgagg | atttttgcat | caatgttcat | caaggatatt | ggtctaaaat |
| 218341 | tctctttttt | ggttgtgtct | ctgcccggct | ttgRtatcag | aatgatgctg | gcctcataaa |
| 218401 | atgagttagg | gaggattccc | tctttttcta | ttgattggaa | tagtttcaga | aggaatggta |
| 218461 | ccagttcctc | cttgtacctc | tggtagaatt | cggctgtgaa | tccatctggt | cctggactct |
| 218521 | ttttggttgg | taaactattg | attattgcca | caatttcaga | gcctgttatt | ggtctattca |
| 218581 | gagattcaac | ttcttcctgg | tttagtcttg | ggagagtgta | tgtgtcgagg | aatgtatcca |
| 218641 | tttcttctag | attttctagt | ttatttgcgt | agaggtgttt | gtagtattct | ctgatggtag |
| 218701 | tttgtatttc | tgtgggatca | gtggtgatat | cccctttatc | attttttatt | gtgtctattt |
| 218761 | gattcttctc | tctttttttc | tttattagtc | ttgctagcgg | tctatcaatt | ttgttgatcc |
| 218821 | tttcaaaaaa | ccagctcctg | gattcattga | ttttttgaag | ggttttttgt | gtctctattt |
| 218881 | ccttcagttc | tgctctgatt | ttagttattt | cttgccttct | gctagctttt | gaatgtgttt |
| 218941 | gctcttgctt | ttctagttct | tttaattgtg | atgttagggt | gtcaattttg | gatctttcct |
| 219001 | gctttctctt | gtaggcattt | agtgctataa | atttccctct | acacactgct | ttgaatgcgt |
| 219061 | cccagagatt | ctggtatgtg | gtgtctttgt | tctcgttggt | ttcaaagaac | atctttattt |
| 219121 | ctgccttcat | ttcgttatgt | acccagtagt | cattcaggag | cagattgttc | agtttccatg |
| 219181 | tagttgagcg | gctttgagtg | agattcttaa | tcctgagttc | tagtttgatt | gcactgtggt |
| 219241 | ctgagagata | gtttgttata | atttctgttc | ttttacattt | gctgaggaga | gctttacttc |
| 219301 | caactatgtg | gtcaattttg | gaataggtgt | ggtgtggtgc | tgaaaaaaat | gtatattctg |
| 219361 | ttgatttggg | gtggagagtt | ctgtagatgt | ctattaggtc | tgcttggtgc | agatctgagt |
| 219421 | tcaattcctg | ggtatccttg | ttgactttct | gtctcgttga | tctgtctaat | gttgacagtg |
| 219481 | gggtgttaaa | gtctcccatt | attaatatgt | gggagtctaa | gtctctttgt | aggtcactga |
| 219541 | ggacttgctt | tatgaatctg | ggtgctcctg | tattgggtgc | ataaatattt | aggatagtta |
| 219601 | gctcctcttg | ttgaattgat | ccctttatca | ttatgtaatg | gccttctttg | tctcttttga |
| 219661 | tctttgttgg | tttaaagtct | gttttatcag | agactaggat | tgccacccct | gccttttttt |
| 219721 | gttttccatt | ggcttggtag | atcttcctcc | atccttttat | tttgagccta | tgtgtgtctc |
| 219781 | tgcacgtgag | atgggtttcc | tgaatacagc | acactgatgg | gtcttgactc | tttatccaac |
| 219841 | ttgccagtct | gtgtctttta | attgcagaat | ttagtccatt | tatatttaaa | gttaatattg |
| 219901 | ttatgtgtga | atttgatcct | gtcattatga | tgttagctgg | tgattttgct | cattagttga |
| 219961 | tgcagtttct | tcctagtctc | gatggtcttt | acattttggc | atgattttgc | agtggctggt |
| 220021 | accggttgtt | cctttccatg | tttagcgctt | ccttcaggag | ctcttttagg | gcagtcctgg |
| 220081 | tggtgacaaa | atctctcagc | atttgcttgt | ctataaagta | ttttatttct | ccttcactta |
| 220141 | tgaagcttag | tttggctgga | tatgaaattc | tgggttgaaa | attcttttct | ttaagaatgt |
| 220201 | tgaatattgg | cccccactct | cttctggctt | gtagggtttc | tgccgagaga | tccgctgtta |
| 220261 | gtctgatggg | ctttcctttg | agggtaaccc | gacctttctc | tctggctgcc | tttaacattt |
| 220321 | tttccttcat | ttcaactttg | gtgaatctga | caattatgtg | tcttggagtt | gctcttctcg |
| 220381 | aggagtatct | ttgtggcgtt | ctctgtattt | cctgaatctg | aacgttggcc | tgccttgcta |
| 220441 | gattggggaa | gttctcctgg | ataatatcct | gcagagtgtt | ttccaacttg | gttccattct |
| 220501 | ccacatcact | ttcaggtaca | ccaatcagac | gtagatttgg | tcttttcaca | tagtcccata |
| 220561 | tttcttggag | gctttgctca | tttcttttta | ttcttttttc | tctaaacttc | ccttctcgct |
| 220621 | tcatttcatt | catttcatct | tccattgctg | ataccctttc | ttccagttga | tcgcatcggc |
| 220681 | tcctgaggct | tctgcattct | tcacgtagtt | ctcgagcctt | ggttttcagc | tccatcagct |
| 220741 | cctttaagca | cttctctgta | ttggttattc | tagttataca | ttcttctaaa | tttttttcaa |
| 220801 | agttttcaac | ttctttgcct | ttggtttgaa | tgtcctcccg | tagctcagag | taatttgatc |
| 220861 | gtctgaagcc | ttcttctctc | agctcgtcaa | aatcattctc | catccagctt | tgttctgttg |
| 220921 | ctggtgagga | actgcgttcc | tttggaggag | gagaggcgct | ctgcgtttta | gagtttccag |
| 220981 | tYtttctgtt | ctgttttttc | cccatctttg | tggttttctc | tRcttttggt | ctttgatgat |
| 221041 | ggtgatgtac | agatgggttt | tcggtataga | tgtcctttct | ggttgttagt | tttccttcta |
| 221101 | acagacagga | ccctcagctg | caggtctgtt | ggaataccct | gccgtgtgag | gtgtcagtgt |
| 221161 | gcccctgctg | gggggtgcct | cccagttagg | ctgctcgggg | gtcaggggtc | agggacccac |
| 221221 | ttgaggaggc | agtctgcccg | ttctcagatc | tccagctgcg | tgctgggaga | accactgctc |
| 221281 | tcttcaaagc | tgtcagacag | ggacacttaa | gtctgcagag | gttactgctg | tctttttgtt |
| 221341 | tgtctgtgcc | ctgcccccaR | aggtggagcc | tacagaggca | ggcaggcctc | cttgagctgt |
| 221401 | ggtgggctcc | acccagttgg | agcttcccgg | ctgctttgtt | tacctaagca | agcgtgggca |
| 221461 | atggcgggcg | cccctccccc | agcctcgttg | ccgccttgca | gtttgatctc | agactgctgt |
| 221521 | gctagcagtc | agcgagactc | cgtgggcgta | ggaccctctg | agccaggtgt | ggaatatagt |
| 221581 | ctcgtggtgc | gccatttctt | | | | |

Following is a NID2 genomic nucleotide sequence (SEQ ID NO: 2).

| >14:50462485-50525750 | | | | | | |
|---|---|---|---|---|---|---|
| rs3742536:32929 | | | | | | |
| rs75305:34983 | | | | | | |
| ctttttacct | gttgggcagt | aggggtagac | tgcagttatc | ccgtagaggt | gagatcgttg | 60 |
| ttctgggaga | tactcatcag | taaactggcc | actatgttta | tttactgata | caacaccatc | 120 |
| cctggtaaca | gaaaggaaga | gtgaattcag | ggacagtcat | ttactagata | aagaagtcag | 180 |
| tcagccacag | aaaatcagtt | gcaatagagg | aaaatttctg | gcagccttct | ctgtcccagg | 240 |
| gctggtgcta | aagccatact | gaagtttgaa | gaccattgct | ctaaatccat | tgctcatctc | 300 |
| tagctgcatg | ttagaatcac | atgatgagct | atcaaatcag | agttgtaggg | catggtgtaa | 360 |
| agggcttaga | ctttaatgtt | ccacagttcc | tcatttgaga | actagtctaa | atagtatttt | 420 |
| tcggtccctc | agacaatatg | tccacagtgt | caaaagccaa | cttaagccct | gtaatatagc | 480 |
| tcatggtatc | aagggtagtc | ttattctcta | aagaacatat | ttagattaat | atgctccctt | 540 |
| ttgagacatt | atccaatagc | tttgctactt | tttaagctat | atgtgagcaa | taccattcga | 600 |
| tttctgggtg | aagtaaaagg | atgatttcaa | aaacatgaaa | ggatgaaaaa | catccttgaa | 660 |
| ggatcttatc | tgccaatgag | gaggtgatga | aacaaaagcc | tcagagggct | taatgagtca | 720 |
| agtcaggtac | tgacttttgg | taaaacaagt | ggtgtgtcct | ctggaacagt | tggccttttc | 780 |
| aggcttgtcc | agaatttgtg | gggctcacct | cctccagtct | gtgtggtaga | agtgatctgc | 840 |
| atagcttacg | atgctgaagg | ggtacttgag | gttgttttga | atgacacgcc | gtccagttcc | 900 |
| atcaggtagt | gtacactcca | gttttttggt | tccttttaaa | acaaaggggg | aaaatgaggt | 960 |
| ccttcagctg | ctttgccaaa | aatgatagtg | acatagtgat | agtgacacag | tgatagaatg | 1020 |
| gggaaatagg | atattttact | tccattacag | tcatagatta | gcaactgtaa | aattacctgt | 1080 |
| cctattacta | gtctccagtt | tttagtagag | attcaaactt | tcaatccttt | tttggaagac | 1140 |
| aggattgcat | ttactgaaat | ctggtaagtt | tctgccaaag | tagggcatta | aacataatta | 1200 |
| tttttataat | ttgtgtgatt | tcaagcctta | gcttataaat | tataacatct | gggtaaatac | 1260 |
| ttgccctttg | taagctatgt | ctataattac | tgaggcaacc | ttttaaccaa | aatccttagt | 1320 |
| atttttaaaa | tattttctag | gtaggaggaa | aaaagtgact | aaaatggtaa | cataactcat | 1380 |
| atttcaatgt | aaaggaatca | gtcttaaaaa | tttttacaat | agctaaacca | ttcaagaatt | 1440 |
| tccaagtttc | acatttctca | tggagccgat | ttaaatttca | aatcctttta | agaaatgtaa | 1500 |
| cttgttttaa | acaactgggt | tataaaagtg | acctatgtac | attaggccct | tttattagat | 1560 |
| tacaaattca | agaacagtct | ttttcatatt | ctttagtata | gaactagaca | cacagcattt | 1620 |
| tttcagttgt | gctgataaaa | gcaaacaaat | gtaggttgct | aatacccatg | ttattactga | 1680 |
| aggaagctac | cctgctacaa | tgctttccaa | agaatgtcct | tagcattata | acagatgttt | 1740 |
| ataggtaagt | tatagtgacc | ctctccccgc | ataagaataa | cttcacttaa | gtttgtcaat | 1800 |
| tcaacaatgg | ctaattcttt | taactgttaa | aaatatgcca | ggcactcatg | gtcaggcaag | 1860 |
| cagtacaaac | ttactgcttg | atatatcctt | ccctccaccc | aaaccccaga | aagttcattt | 1920 |
| taagactcat | ttactagtac | ttaaatttac | tagtacttaa | aagtttacag | accaaagaaa | 1980 |
| ggagatctaa | gtgatgggat | ttcattttgt | agtaaaatct | gttagtgctc | acattcactg | 2040 |
| tgatgagagg | ttatctattt | gcattccatc | agtagtatta | cctgcatctg | cccagcagag | 2100 |
| cagtttagag | aaagggtcaa | aggttaagcc | attgggcaat | ccaatgtctg | tattgatcag | 2160 |
| aattcttctg | ttttctccat | ctaaagatga | cgtttcaatt | ttaggagctt | ctctattcca | 2220 |
| gtctgtccag | tacaagttgc | tgtaagttaa | aaatcaagat | tgtaaaagaa | tagccatgta | 2280 |
| gcctgtggta | ggcagcatct | aagcagcccc | cagtgatctc | catctcctca | tgtattatag | 2340 |
| ccttaagcaa | tccccttgag | tttgggctgc | attagtagtg | tcttgcttct | tctagtgcat | 2400 |
| agagtatagc | agaagtgata | ggctatcact | gccgatattc | ggtctcaaaa | aactggtttc | 2460 |
| tgccttaggg | gctctctctt | gctccctttg | agggaagctg | gatgccatgt | tgcaagctac | 2520 |
| cctgtaaagg | ggaacatgtg | ccaaggaact | gatgtccttg | agggctgttg | gccagagaca | 2580 |
| ttagtcttga | ctgtagccca | tgtgagcatg | cttgggctgt | gtcttccccg | agctgagcct | 2640 |
| tgtgatcact | gcagtttggc | cattacctga | ttgcagcctt | gtgagagact | ccatgcctga | 2700 |
| gggagctcgg | tgaagacaca | cttggattcc | tgactcacag | aaactgtgag | actaaatgtt | 2760 |
| atcttacact | gctgagggtt | tgggcaattt | cttacacagc | agtaggtaac | taatacacag | 2820 |
| ccatattcca | cttttacctg | tatcacaagg | ttttctgatt | attgtcacta | tgtgggagaa | 2880 |
| gtcattacta | agttactatc | agaatccctg | cctccaagat | accccttgac | tcaactgccc | 2940 |
| caaaaccctc | cgtttgttga | agtcttgaat | tggatcttgg | gttttagaat | agaaacaaat | 3000 |
| acagctgtta | ggaatggctt | cgatacaaac | tatggaactt | gaagagatgt | ggaccaggga | 3060 |
| tttgagaagg | aaactgttct | ttcttctgcc | aaaaatacaa | tttggtactt | gaaggggtac | 3120 |
| ctggcaccca | gggatggaaa | cattttaagt | taagcacttt | accttttaaa | tgccacgtta | 3180 |
| aaaatgaaat | atattcataa | cagatactaa | cgaggaaata | tggtcctatt | aagtggaaat | 3240 |
| aggttataag | gaaacaaaca | ggtttatcgt | tagagcagaa | ataatgttga | aagcactttt | 3300 |
| tataaaatac | tattttataa | aagctataga | agctttgaat | aaatcagttg | ggctacagag | 3360 |
| aacctcagtg | agaggagagg | aagaaaccat | aggtttcccc | tgttgcgaat | ttcatccctt | 3420 |
| atgcccaaca | gatgaagcag | agagaagctg | agagcaaaag | ctagaaagca | gtttccactt | 3480 |
| tgcgctaagg | tggaggtggc | cagagggtac | agcatgcata | tcattgcttc | atcgcttctg | 3540 |
| atcagtctcc | tccccaaaat | gatttaatat | catcaacaac | aataagtctg | aaacaagctc | 3600 |
| acacagatat | ttggtgtaaa | ttatttgggg | attgccagag | gaagaaagtt | gtaaaggtga | 3660 |
| aatctataga | aagcaaatga | gaaatatgca | ggaaggcttg | acaaccattc | tgttacgcag | 3720 |
| ttgaaggaga | tgtaattaca | tgtgttatat | tgcaacctgg | ttcaaggtgg | gttttctttt | 3780 |
| tattaaaaat | tcaagtcctt | tagcatttat | atttgctgtc | tggataagaa | agttctactt | 3840 |
| tcatatttaa | actcatggct | gtgatgtgga | tttcagctta | aaagtatgct | ttttccccag | 3900 |
| actccagaga | catggccaaa | gtttcattgc | ctacaagatg | gcagctctcc | ccagttttca | 3960 |
| ggtcaagcac | caagaaggaa | taaggggccg | ggcgcagtgg | ctcataccta | taatcccagc | 4020 |
| actttgggag | gccaaggcag | gtggatcacc | cagggtcagg | agttggagac | caaaatggcg | 4080 |
| aaaccctgtc | tcgactaaaa | atacaaaaat | tagctgggca | tggtggcaca | tgcctgtagt | 4140 |
| cccagctact | tgggaggctg | aggcaggaga | attgcttgaa | cccgggagat | ggaggttgta | 4200 |
| gtcagccgag | actgtgccac | tgcaccccag | cctcggcaac | aaagcaagac | cttgtctcaa | 4260 |
| aaaaaagaaa | aaaaaaagaa | aaaaggacct | tccaaatgtc | caaatgtcag | aaaggcaagc | 4320 |
| ctatgcaaag | agtttttaag | agggctatta | attcctgtta | agtcaactgg | aatagagaag | 4380 |
| gatacaggta | cttttcctgt | gtgtagaacc | caaagtttga | aaccaccttt | gaggcactgc | 4440 |
| actaagtatt | tcatagtcaa | aaagcactta | cctgaaaatt | aaagctgaag | gttttcttta | 4500 |
| ctgatttgaa | gggcatctgg | gaatttcatt | tcgtagaaac | tccacttaaa | catggcaaga | 4560 |
| gtttctcaag | atataaaaat | tctccctgat | tgtaacaagt | actgagacag | tgaacaaatc | 4620 |
| ctgtatgtca | catttgatga | ccttagcaat | tatacagaca | gggaaaatgt | ttatcttgat | 4680 |
| ttctgctaca | gcaacactga | agccagaggt | gtctgagctt | tggctaatgt | cacttgatgt | 4740 |
| aggtcagccc | tactgtgagc | gtcaaccctg | gtttctaaat | gtgctgctgg | aggaatccca | 4800 |
| cttcacttcc | agtttaagtg | tcagctgagg | cctcgcctga | tcatttgatt | cagagtagcc | 4860 |
| ctagttctca | caacactatc | tgaattttct | acatatcaca | tcacagacta | atattgttta | 4920 |
| taccagtctt | gtttcctcct | aataaaatat | aagtgccatg | aaagaacaga | ccttgtcttt | 4980 |
| tcctagaaca | ggtcctgaca | cctcttagat | cctcagtaaa | tctttgttac | atgaatgcat | 5040 |
| ttgagctttc | actctcttgt | tctgaccttc | cccacattgt | atttaaaacc | caagggcttc | 5100 |
| acatcaggat | ctacaggtaa | gagaagaatt | agggaagccc | agctgaccct | cggattggat | 5160 |
| ccacagcgat | ggcacgggga | ttcaccagat | ctgtgtagaa | gaggaccttg | cgctcagagc | 5220 |
| ctaccagttt | gatgacaacc | ggcatccaca | gtgccagagc | caggctgaat | aaacgaaggc | 5580 |
| gtcaccctga | attcttacca | tgaattcttt | taagaagtgc | gcacagtagg | aatcacctat | 5640 |
| ttcattccgt | atgtatgaag | gtgtttgctt | acagaggggg | ctagtcttat | acagctcaat | 5700 |
| aaaatgactt | gcctagaact | taacctcccc | taatggagaa | aaatcgaggg | gagacttcgg | 5760 |
| cgtaaagtag | acaagtgact | ttgtagaatc | aaatgaaatg | cagactgctg | agtgaagctg | 5820 |
| ctgacctgaa | ttcacgatcg | tctcaggctc | tgctcccagt | tccagaccag | cacggctgat | 5880 |
| tgtccgtcca | gcaacatctg | tccagtacac | catcctctcc | cggcagtcgt | aatcaattcc | 5940 |
| cacgattatg | gagccctttg | tgcatcaaat | catagaatta | gaagaattag | gttacatttc | 6000 |
| ccctttgttc | acactcagct | gccttgctca | tgcacagctg | cagtgagcaa | cactgcactg | 6060 |
| tgatcctgca | ggcaacagag | cagagtagct | ggacatgtgg | gcactcgggt | gaaatctagg | 6120 |
| ctcagccact | tagtagccat | gtggctttgg | gcatttaacc | tctatgcgtt | tgtctcattt | 6180 |
| gcaaaatgag | gccaatagtg | tacctgcctc | atatggttgc | tataaaaatt | aatactataa | 6240 |
| gtgcttgtaa | agtgtcctaa | tcagtaattg | gcatacagta | agcccttcaa | taggtgctat | 6300 |
| tatgactgat | gatttaaaaa | aaaaaaaaaa | tcagcccctt | cagtaattag | aggacatggc | 6360 |
| cttaagcaaa | ataagttttt | aatattttac | tgggtaaaaa | ctgaatcttt | tcccaatgtc | 6420 |
| atcagcactt | ttcccagagc | cttaataaaa | tttgccacta | tatgccaaca | aaatctactc | 6480 |
| tcccctcctc | tgtgaatgag | atctttcctc | tacctgaagt | acaatcacct | atactcctgc | 6540 |
| ccctgactct | taggaggcaa | gatgttggtt | atatcctgta | ctaaagaaag | tgtagtcagg | 6600 |
| cacagtgact | cacgcctgta | atccctgtcc | gttgggaggc | caaagcagga | ggattgcttg | 6660 |
| agcccaggag | ttcgagactg | agcaacatag | tgagaccccg | tctaaaaaac | aaaaaataaa | 6720 |
| tgaactgggc | atggtggcac | gcacctgtag | tcccagctac | tcaggagacc | gagttgggag | 6780 |
| gatcgcttga | gcccaggaag | tcaaggctgc | accaagccga | gatggtgcca | ctgcactgca | 6840 |
| gcctgggcaa | cacagagcta | caccctgtct | caaaagaagg | aaaaaatcta | atttattggg | 6900 |
| ttaaggaact | agggagtagt | ggttatccaa | cctctggcta | gtgaggtctc | tcctcctcat | 6960 |
| tgctaaataa | atgacaagtc | ttttaaaaaa | ataataaaaa | taaaaaaaat | gctgagtctg | 7020 |
| atatttttcc | tcaactccat | gcagaacttg | cttcattcag | tttccaatcc | aggctcctgc | 7080 |
| tctgagaagc | ccagcagatg | tgcaaagcca | acattcaggg | gttagctggc | tgtccctgag | 7140 |
| gtcatacaag | ttcctgcctt | gcctaacagc | ctcctggggc | ttggcttcac | ttacgccccc | 7200 |
| tgggagcgta | gtcagccaag | aaagattgta | caaaacggga | tttggcttca | gtggaaaaac | 7260 |
| aggcagctta | tatggggggc | gggcctaagg | tcaactacac | ttttatttga | aaattgaaaa | 7320 |
| gtcgtttgtc | ccaggcaaga | gtacacattc | aaatgagctt | ccattaaccc | gggaatcatg | 7380 |
| ggattcataa | acccatgtca | taacctcagg | ttagagaaaa | agtcccagtt | taagtaaatt | 7440 |
| tccttaaaat | gtgcaaataa | cccacgtccc | atttctttca | gttccactac | cactaccatc | 7500 |
| accaccctgt | cctggtcagg | ggcatgttca | gaaaggcaat | caagctctat | gacttgaata | 7560 |
| aaagagggtt | agcttaaagt | gatgtcaact | tcttagtctc | tacctcttcc | agagaaagtt | 7620 |
| ttacaaaaca | atgtgagagt | tttcttgggg | gaggggacag | gacccaacaa | actcgaatct | 7680 |
| taaaaagaac | acatctcttc | ctaaacactg | gctgcctcta | agcttttctg | gaaccaaata | 7740 |
| agtgtcgttc | attaacagaa | gtaaatgtaa | acttgaatgc | cgtactattc | tttctcaggg | 7800 |
| atgagcaagt | gcctggagtg | tgaagttcaa | caatggaatc | agatgatttt | ccactgtgtc | 7860 |
| gatgccattc | gagacagcca | cccttttttt | tcctccattt | ttgagaaaac | ccgctcccca | 7920 |
| aaatggggtg | ccttgcatta | gggaggctgc | tggctcagga | agcatcaaac | atcccctggg | 7980 |
| gaaggctcca | tccactcctc | tcccagcatc | acacactcct | caagttttca | tctatggagt | 8040 |
| cagggaaggt | taggaaagaa | gtgggtcaaa | gggacagaac | caaagtgaaa | gggaagggct | 8100 |
| gcaacggtga | gacagtcatt | aactgacttc | cacttggagt | ccccaacaaa | ccgcagagat | 8160 |
| cacaccttgt | gactagttgt | cctgtcactt | tgatttccag | gatttccatg | gagatgttat | 8220 |
| gacatacaac | cctggggact | tagggtaaga | gtcggtaaga | tttcccttgg | atcaccatac | 8280 |
| agcctccatg | tgctgacaac | tctaaagttc | cctttccata | ttatctgaaa | ctccatgaac | 8340 |
| gaaccagaga | gcacagcctt | ggtcagtgac | aggtgactcc | cagcaagaga | gagccctggt | 8400 |
| cctatggtgg | tggcatcggg | cccatgccga | tgggctgcag | ctcagaaacc | ctccaggact | 8460 |
| tccctgcacc | agtccacctc | actgcaacag | ggcctgtgag | gtggctccca | ctgggaaagc | 8520 |
| cacgcagccc | tgccccctac | aggagaaatc | cactttacat | gcagagacag | cagggtctta | 8580 |
| gctgcatcct | tctgaagcct | ggtgccattg | aggggtaagt | agccaatctg | ctggccctga | 8640 |
| gtatagagca | ggaaggtgcc | cacagatgga | ggggtcacat | ctggccgggg | cgtgggccgg | 8700 |
| accatgggtg | gagcgacggt | gggtatacct | gtgggagaga | gggtgggaga | gcaggaaggg | 8760 |
| gaacaagggc | aggcagggag | atgggaagaa | aagttacttt | accacatacc | agagcctcca | 8880 |
| ctaatttcta | gggttttttt | aaggatcaat | tatgtaaatc | tgccatttac | tagggaagaa | 8940 |
| gacagcaaaa | cacttccctc | agaacaaagc | agaacagatt | tttttttcaa | gagaaaaaaa | 9000 |
| aaacagagcc | agcattgtgt | cagcttcctt | ttaggactcc | agagtgacac | gtgtcccctg | 9060 |
| tcaccctggg | ctggggaggg | ggagtaactg | cctgctgaac | cgagttgcag | atgttccaat | 9120 |
| tatggaagct | gctaatgaca | aaaaccagac | accaaacgca | aattgggatc | ttgtgtggca | 9180 |
| cagattataa | atcaacatag | cacatagtga | cttctttcat | tagacctggt | gaccccacat | 9240 |
| gtccccccac | ttcaccacag | caggcaccat | ccctagcaaa | ggccttagat | acagctgagg | 9300 |
| ggagcggggg | cggccaggtg | cttacacgca | ggggtggtgc | ctggctggga | gcgggtgccc | 9360 |
| tgcacctctc | tgccatcttt | gtccacacac | cagcagaagt | cgctctttcc | gtggcactgc | 9420 |
| agggggatga | agtggcccag | gtcatcgcac | tggggcacgt | actggtcatc | ccggggggtg | 9480 |
| ccaccgtagt | gctccagcag | gttttccctc | cagcgctcac | agatggtcgg | gggcctctgg | 9540 |
| gtgggctctg | agcagatggg | gaagagggaa | gaagaaaaac | ctttgattgt | gagtcaagga | 9600 |
| cagaatgcaa | aatgtagctc | aagaccccat | gggtggcctc | ctggccttcc | ttctcctact | 9660 |
| gtccaggtct | cagccaagcc | cgtggacacc | aaacttggga | agcaaatgtg | ttctaagtac | 9720 |
| ttctccacca | tgggaatcag | aaatagatca | tgtgatgctt | ctttcttgcc | aggagggtct | 9780 |
| gtcatcaagg | cccagccaca | tcaagaaaga | acaagaggag | gtaagatcag | cagtgcagct | 9480 |
| cactcagcct | gggaaggggc | atgatctcct | tctggccagg | agaggcccac | aaggcccatc | 9900 |
| ctgcctgtac | ctcgggcgcc | tctgctgaca | gagcacttgt | gcaggtgatc | cttggggact | 9960 |
| cagtagccga | ggtgatgctt | aaagccaatt | tgtattcatt | ccccacttgc | ttcaagcttc | 10020 |
| aagctcgaag | ctgctgagga | gagggccatc | ttgcgactgg | ccagtgtgtg | tcttagaaag | 10080 |
| tattactgaa | agcccagcgc | cacggtgccc | tctgtctacc | cactggaaaa | ctacatggag | 10140 |
| taaaggtaaa | actgagtgga | attagtagta | aagcatagca | gaggtggtgg | caaacagctt | 10200 |
| aaaaatggtt | cttccacagc | ccagtttcca | gaattgcttt | agccaccctg | cgcctgggat | 10260 |
| tatctcaaag | ctgaatcttt | tcctgcaggc | tctagcttcc | tgccgtgttg | ccctttcccc | 10320 |
| ccctttctcc | agcatcctct | cctcttgtcc | cctccctcac | agctcactcc | atggttgaag | 10380 |
| tcctgctggc | tttggttgca | aagcagtttc | ctcccaggct | gcttccacag | tgccattgag | 10440 |
| gctttgtcca | aaatacaggt | tgttctgaga | agactctgct | gcagaaggtt | attgagataa | 10500 |
| cttgttcatt | tatgttagga | ctacccaaag | gaacttaaac | aagaggaggt | gcttctcctt | 10560 |
| cctttttaat | tcctgcaact | taaagcacat | ggtctgcaga | atcacatagc | caataaccct | 10620 |
| acttgctacc | accatttaga | tgtataatag | aaatcagaaa | ctaatagaaa | ctgagcccct | 10680 |
| gccttcacca | tcaaaccagc | tgctcccctg | gtctttccca | tatgagtaaa | tggcacctcc | 10740 |
| atttttccac | tgctcagacc | taattatcct | tgaccctcct | ttctctcacc | atccacattt | 10800 |
| gatatgtcaa | aaactctttt | taggggtaac | ttcagaatcc | acttagaata | tctttacttc | 10860 |
| tcactactgc | accccctcac | cctggtcaaa | aagcctcatc | ctctccctgc | aatttctgca | 10920 |
| acagccttcc | cactggtttt | cctgcttcca | cccttctatg | ccctcactct | ctccaaaacc | 10980 |
| agtgtaaatt | caattagcag | cagccagaat | ggcccatttt | aaacataaag | gcatttcatg | 11040 |
| tcactccgtt | caaaaccctc | cagtgactct | aaagaacaaa | gctgggagca | ttatgatcat | 11100 |
| gttccagcac | ctacaattcc | tcttggaaca | caaaagactg | cgaatgaagt | tgtcagagct | 11160 |
| caaacccaac | tagctctagc | tcaactgcac | gactcaccag | aattggggga | agcgaatgcc | 11220 |
| gttaagttgt | cactgtagct | catgacccat | gggagacaaa | gctcttttta | agaccccgtg | 11280 |
| gagaaggtaa | gactctgact | tcttcagcca | aatgtgtctc | aaggaacaca | gagagggaaa | 11340 |
| tgctacttcc | tcaaacactg | caggatttcc | tggagaagtt | cccaattcca | cccctgctcc | 11400 |
| acattcagaa | ctccaaggat | ccatttttgc | tttaagggac | caccctgtgc | tatgtgtctg | 11460 |
| cattccaaac | aaagagacat | gcctaactcc | tgggaaatgg | gtgtcccaaa | tcactcatac | 11520 |
| atgggcatcc | ctttcattcg | ttactatttg | ctacttttca | acccaaccat | gaatttcttt | 11580 |
| ctcttgtgct | tctggttggc | aagggtttaa | gtattttgaa | attgtgggca | cagagcgtcc | 11640 |
| ttaccctccc | tcaagctgac | cccatcaaac | atgagaccag | tcagagatag | catggggggc | 11700 |
| caaaccagcg | ccgagagagt | gtgggcagga | aaaagtccgc | acaagagaac | acgaccctat | 11760 |
| tttcgtcatt | cacttctcag | tctcttggtt | cccccccctt | taaaatcttg | gttccccctt | 11820 |
| taaaatcatt | gtccttaaca | catgcctgtc | ttaataaatt | taatcgaatt | ctgaaccatt | 11880 |
| ccccaaaggc | acctcccaac | catttgattt | cttcaaatcc | cagatggagt | tagaaccaag | 11940 |
| acattttcaa | agagaaggca | tgagggaaat | agaatttctc | tgtacctttt | ttggaccttt | 12000 |
| aaaatctttt | cctaaaaata | gaaacaaaga | gcgactatgg | atcaaaccat | taccaatttt | 12060 |
| tacctatctg | agaaccgcca | agacctgacc | tttttttaaa | aaaacaaaaa | aaacagagtc | 12120 |
| tcactctgtt | gccaggctgg | agggcagtgg | catgatcttg | gcctactgca | acctctgcct | 12180 |
| cctgggttca | agtgattctc | ctgcctcagc | ctcccgagga | gctgggacta | caggtgcgtg | 12240 |
| ccaccacgcc | cagctaattt | ttgtattttt | agtagagacg | gggtttcact | atgttggcca | 12300 |
| ggatggtctc | aatctcttga | cctcgtgatc | cacccgcctc | ggcctcccaa | agtgctggga | 12360 |
| ttacaggcat | gagccaccat | acccgggcaa | cagttaacac | cattaaaagg | gaaaaaaact | 12420 |
| attggtaatt | ctccagttct | tcttactttc | acacattaaa | agccaagact | agtatttatg | 12480 |
| gaacccttct | caatagtaaa | aatggatctt | tggatagcaa | gctgtgtcgg | aatcattttg | 12540 |
| gctaaaacgg | tatcaagcca | atcctatgct | cttcattaaa | taaagttctt | ctgtaatgat | 12600 |
| taaagaaaca | agttttacct | atccctgaat | aattcaacaa | ggaacagaaa | gttgccttat | 12660 |
| ctgcctgctg | ttggaatcga | ctgtgagcac | ttgatcacca | tgccccaccc | agctggggaa | 12720 |
| tgctctagat | ggtagcaggc | atgttctcag | gttgtatggg | ctaacagaaa | ccagatgtct | 12780 |
| gtgcctcctc | tgagatgttg | ggctgggtta | cagagggctg | ggattgtcag | tttagagaaa | 12840 |
| actaactggc | tccactagca | ggtgttattt | ttctaatttt | tcctattgat | actaacagag | 12900 |
| cgttcatact | gtttcctgcc | attcagaagg | cagaggtaag | tgagatagtt | acataggaaa | 12960 |
| gcatttttcc | ttctggtcaa | aacgccagtc | tggccagatc | ttctaactgt | cagatctccc | 13020 |
| taagctgctc | tataacaggt | ctagggtagt | gttctcagtc | tgacctacat | acacaatctc | 13080 |
| aactttcagt | ttgtactgga | caagggtcta | agaggcaaat | gtgtaacaga | tcatatgtgt | 13140 |
| catgtattgc | atatctctat | gtccttatga | cacaaacatg | catatttgta | cgcactatta | 13200 |
| ttcaagtctt | ctcaggagga | aagaggctat | gaaggatggt | gactggcccc | gtgcctgcgt | 13260 |
| ggtctgggca | ggaattatga | tctacctacc | accagtgcca | ttctgcttct | tgagccccag | 13320 |
| gcctaagctc | acctggtgat | ggtccacagt | gaggcggggt | ggagccaggt | ggagtctggg | 13380 |
| taccaggaac | ttcatgacca | tcagggtcca | cgcaccagca | gaaaccagtg | ctgccatgac | 13440 |
| actgtagggg | caggaagttg | ccctgctcgt | cgcattgggg | gatgtggaac | cgggccccag | 13500 |
| ggtaggcata | ctgggcctgg | gcatggcgct | gctgttgttc | acagggtgtc | aggcttgagg | 13560 |
| tggagtcttc | caggatcaag | gcagaggaag | acacaaaaga | gaaatagaag | gcattgcaac | 13620 |
| ccatccactt | caccagccca | cagcgtggca | tggaaaagcg | ctgggatttt | ggagcccgag | 13680 |
| attcttattt | tataatacct | tgccactatt | tacctcattt | ctctccagtt | cttctagttc | 13740 |
| atcctatagg | tccctagctg | gctctaagtg | taaagcagcc | cggcccctcc | ctattgtcat | 13800 |
| ctggggaaaa | tgagggatag | cggtggcatc | tgcccaacat | cccaagagac | tctggagaaa | 13860 |
| acaagccaaa | gctcattcat | gccctgaggc | ccacaggtta | agtctgagaa | gctgcaagcc | 13920 |
| aacggcactc | accactcacg | agactgcacc | aagctagcca | agctcctcta | acacggtcgc | 13980 |
| cacgggctta | accctggcta | ccggcgcata | aggctctgct | taataagctt | gatctcaaca | 14040 |
| cttcatccaa | attgcactca | tcaaggtcaa | gcagatcact | agggcagtta | accagagggg | 14100 |
| ctatgaggta | ggactatggg | tgggtcgtct | cccgcaggcc | tccagaaatg | ttacgaatca | 14160 |
| gtgaactatc | agtaaactag | gcagcaggag | gtaaccaagg | aaaaaaatcc | acagcttgga | 14220 |
| aaatccacca | aggctccaga | ccaggctaaa | tcaagagtgg | gctgtcatag | aaaaatatcc | 14280 |
| ttgaaggagc | taagagattc | atgtgcctaa | tctggccctc | tgaaacttac | caggtatgca | 14340 |
| ctgaaatcca | tccccataat | atccgggttg | acaacggcag | gagaaggaac | caggagtatt | 14400 |
| gtagcaggta | gctgcagggt | gacatctgtt | ttctgagcat | tcatctacat | ctgcagaggt | 14460 |
| cagaaacaga | agaaagaagc | aaagaacact | atgacttcac | tcacacaatc | tgtgcgactg | 14520 |
| catgggcttt | ggatatgtgg | atcaacaccc | agtgaggtgt | cagcttcaga | agacctttga | 14580 |
| gcatgagcag | aaaaatgctg | acacctaaag | gtaagcttaa | tagaaaggaa | gaaaaaaaat | 14640 |
| caaggaacat | gtgagttaac | acactagact | aaacgtgttc | agtgtagaaa | tcaaatgggt | 14700 |
| agtcgctcac | agaaaaaaac | aacacatctt | acagcatgtt | tcattgacag | tgaattcaag | 14760 |
| gaagttatta | cctttgaact | aacctttaga | ctataagtca | ttaacttcct | ttcagcaatg | 14820 |
| ctgcaataaa | gacaaatata | tccaacaatg | gttttttttt | ctttcgttgt | tggcaccatc | 14880 |
| tccctgtaac | attcgtctgt | agatgcaaag | gagccagatg | aggcccaaat | atttttcagc | 14940 |
| taagaagcac | attctgtctt | gactaattag | aatcttacta | tctagaacca | cacccccatt | 15000 |
| aaccaggtat | tttctttact | ttccactttt | agaagaaata | agaaaaaaat | attatctagg | 15060 |
| gttttgaaat | aatacttaca | aaatggaaat | ctaaatttgc | caggcatatc | agtacaaaca | 15120 |
| attcaggccc | ctacactctc | cacatagcac | catgaggatt | aattttcacc | atcatatttc | 15180 |
| tcaaaacaac | aaaatcctga | cacttcaaac | aaaagccaac | ttacattcct | tttatttaat | 15240 |
| gaaatccgca | tatgccgaca | atagcaagtc | acccacccct | cccatcccca | gcagaaaaaa | 15300 |
| agcagctctg | agaattgaat | ggttaaaact | aacgcccaag | ggatgagata | agctacttta | 15360 |
| agagacagga | agtctttcag | agtaaaagga | ggataaacaa | agggtgtagg | tccgattaag | 15420 |
| gcttggctga | aaataaaaca | aagcagtagt | tccatccata | gtaataacca | agtttcttta | 15480 |
| aaatgtgtga | atagaaaaaa | aaaaaatgtg | ggatggtgtg | atcagtgtga | aacctagaaa | 15540 |
| atgagaatgt | gttgcatcaa | tacggtgcag | cccatggtag | agacacatca | gcatgggaag | 15600 |
| acagatattt | gaagccctaa | atttgatgtg | aatgggttgg | gaacagggct | agctcctcac | 15660 |
| ccaggctccc | atgacccaca | tggaacctct | tctagagtta | cctgcaccac | cctttgtcca | 15720 |
| gaccacactg | tcactgggtc | atcctctaat | aactaatgtg | ctttctcgaa | taccattaag | 15780 |
| tacacacact | gggcattaag | cttaggtgtt | ctgttcccta | gagaggaatc | tcttatgtta | 15840 |
| taattagaag | gtaagtattc | tgcttatctt | ctgtctctgg | ggagtttagt | cagcatatat | 15900 |
| catttcctag | gagtcctccc | ccaaaaattc | tgaactcaat | gcaacccata | gagtctccac | 15960 |
| ccaggtgagt | ggttggcctc | cctggatacc | atcatttcgc | ctataaaggc | aagaaagcag | 16020 |
| atatcaattc | atctgcaagg | gtagaaacaa | accagattgg | ctcctcagtt | accctgaaat | 16080 |
| caaccaatgt | tgtcataatt | atccctgagc | ttggctctgc | ctcttcaaca | ggacaattca | 16140 |
| aatgcccagc | tgggcaaaac | tggtgaccat | ccaatacctt | ggacctggaa | taggctgtaa | 16200 |
| aactggctgt | aaaactaaac | ctgtaaaaat | taacattcag | ccgaatgcag | gaaattaaag | 16260 |
| ctattcacaa | tacgcatgct | aaaagtactt | ctaatggtaa | caggaacccc | aagcaatgac | 16320 |
| aggaagcagg | aattcctgag | acacagggcc | tatttctgga | gcccatggat | cagtttctta | 16380 |
| ttgttacttt | tgaactcaag | agagagggga | ttccccagag | ctaccaaata | aatcaacggt | 16440 |
| gtgcttagaa | ccttcagcct | aagaattctg | actcaatctg | aaagaccagc | cttcctcccc | 16500 |
| acagatactg | ttggcatcat | caaagatact | aaagttctgg | gtagacagaa | actattagaa | 16560 |
| atgaccaaca | aggaggagaa | agtccccaaa | gagactgaag | atatcacaag | tgagaaattc | 16620 |
| ctgccccaaa | gattgagact | ccctgaattc | gcagtcctga | ctcctccttg | gagggcctct | 16680 |
| cacaccatga | tagagactag | agtcccggga | cctgggcatg | cacaggacat | cagatgtcaa | 16740 |
| gagagaaaac | atggaaagac | tttcagcccc | ctggcaaggg | aggaacttta | ttggagaagg | 16800 |
| agaaggaggc | cactgcttat | gcttagtcat | tactagcagg | ggcctgtagg | gactgctata | 16860 |
| aagacaaaaa | cgttcaagaa | gtcgtagagc | ccctttatca | ctcaggtaat | gagaggtagc | 16920 |
| ttaatctcag | tgcaatccta | cactctatta | agctagtaaa | gaacctgttt | ggaaaatgtg | 16980 |
| ttaaaaatcc | ttcgagttca | tgctttcgtg | cttcaacatc | cttctcactg | cccaagtact | 17040 |
| gagcaaaacc | agcaggaact | gttggatgat | aaacctaacc | cctggcctcg | agttccccca | 17100 |
| ggctcccgcc | ccttcaccac | tgcccacttg | cctttgagcc | cagtgaaatt | tcttggcatc | 17160 |
| aattaacctc | aaacccatcc | cagatcccaa | taaaggattc | aacccacagg | tcccactctc | 17220 |
| tctctactgc | acaatctggt | caattgggca | ccctgctgat | tgtacccctg | tatggcatgt | 17280 |
| ggtgaaccca | ctccagacct | gtaagtgtaa | taaactactt | cctttcaagc | ttcatccttg | 17340 |
| cctcctccta | taaccacact | gattttacca | taccataccc | aacatctacc | tacattcttg | 17400 |
| gaacaggaaa | cctggccagg | cacataatgg | agatcatgct | ttcgttatca | aagaagagag | 17460 |
| aagctaggcc | gggtgtggtg | gctgacgcct | gtaatcccag | cactttggga | ggctgagggg | 17520 |
| ggtagatcga | ttgagctcag | gagtttgaga | caagcctggg | caacatgatg | aaaccccatc | 17580 |
| tctacaagag | ataccaaaat | tagctgggca | tgctacttgg | gggggctgag | atgtgaggat | 17640 |
| cacttgagcc | tggaaggctg | aggctgcagt | gagctgtaat | cacaccactg | ccctccagcc | 17700 |
| tgggtgataa | agtaagaccc | tgcctaaaaa | aataaataaa | taaataaaaa | ttaaaaaaaa | 17760 |
| ataagaaaag | aaaaaacgcc | aggaaaccca | agaaagaata | atgtcagtat | cttccctcag | 17820 |
| gcccctgagt | aatgatggct | ctgggtaatg | ggccatctgt | aacccagctt | cctcaactca | 17880 |
| cacttgctcc | tcctgggaag | gtcccaactc | aagacaatct | gcttttgcac | ctggccaagt | 17940 |
| cacaacaatc | caaaaggaca | aacatgtctg | agaaatatcc | agctcacacc | aacacaagtt | 18000 |
| tgaaatcact | ccagattgag | ggaagctgca | gaggatttaa | ctctacttgg | ataaaagcct | 18060 |
| cctggctagt | gactcagacc | agactgagaa | cccaaatcca | agtgcagcct | ctccagggag | 18120 |
| ccggtttttt | taacaaacta | tggctggaga | aaaactccca | caccccctta | ttgttagttt | 18180 |
| gatggagtaa | aagagcatgt | gctttaacaa | tgcatagcac | ccacaaaata | cagcctctgt | 18240 |
| tctttggtca | ctgagacaca | tgtaccctat | ggaatgcctt | taaatatgca | cacatacaca | 18300 |
| tatatttcaa | tatttattct | tcaagttact | ttcatgatta | gcctagcctg | atttatgcat | 18360 |
| gggtctttag | gaaaatgacc | attcattctc | tactactcca | caatctgaaa | gtttagaaga | 18420 |
| caactacatg | gtaagtggct | gtggtaggca | gcctctaaga | tggcccccaa | cagtccctac | 18480 |
| ctagtggtat | tcatggtctt | gtgtaatccc | caccctttca | gtaacttgct | tttaattaat | 18540 |
| aaagcaacat | tgaagggatg | tcatttttgt | gattatgact | tccatcttgc | tagcagactt | 18600 |
| tctctggcat | taataaaata | agctgtagtg | ttagagaggc | ccaagtggca | agaaactgaa | 18660 |
| ggcagccaac | aaccagcaag | gaaccaaggc | tttcaatcca | atggtcctca | aagaaccgaa | 18720 |
| tcctgcccat | aaccacgagt | ggatctttcc | ccagttaagc | cttcagatga | gaccactgac | 18780 |
| ccaccagaca | ctttgactgt | ggccttgtga | gagatcctaa | agcacaggac | ccagctgagc | 18840 |
| cattcccagg | tccctggacc | actgatatta | aatgtctatt | gcttgaagct | gctacatttt | 18900 |
| agggtaattt | attatacagc | aatagataac | caatacactg | ggggaagcac | ctgatgggtt | 18960 |
| tggcatattt | ttagtttgta | ggctttggaa | agaatcctaa | aagcagagaa | ccagaaataa | 19020 |
| aatagggaca | attgaaaatg | acaaaaatag | ggcaaattaa | aaaatgacaa | tacttaaatt | 19080 |
| cccaatatgg | aaaagtagtc | aatttcagag | ccctaacatg | accccagaag | gtaatcaaag | 19140 |
| tcttgcagga | caagtagaag | aaaaaaggag | ggagagaatc | attgtagaag | tgatcaaagc | 19200 |
| atgcagcatc | taatctaaat | taatattctg | attaacactc | atcatatcta | ggctgaaatt | 19260 |
| aatttcaact | tctttgcttt | aaaccagaga | gctaaggaac | taaatatttt | aaaagcacca | 19320 |
| tctctgctga | tctgagcagc | cagatgttta | aataaattct | acagacaaga | tttttctcag | 19380 |
| tggattcaca | tttcttagag | ctggtactag | ggttaaacca | aacaaacgat | gtgttccaca | 19440 |
| cgaaggaaaa | gcatcattcc | cactgtcagg | ctttggcacc | tcaccctgaa | ctccatacct | 19500 |
| gtagagggta | tctgtaaaga | gtgtatctat | tatagatata | ctcaatgttt | tctcaccagt | 19560 |
| atatctatga | cagatggagc | tcctggctgg | ttaagtggga | caaacatctg | tgaattacaa | 19620 |
| atctacctca | ttctaattgg | gaaatactct | ttatctagag | ctaggggtgt | ttattcaaga | 19680 |
| ggtccatagt | cttcttacat | tactgggcct | aataccaaga | cacacaggaa | tcccaactga | 19740 |
| gggttaccat | ctgtattagt | caatttcctg | ttgtttataa | tagaatatct | gaaactgagt | 19800 |
| catttctaaa | ggaaaggaat | ttatttctta | tagttctaaa | ggctgagaag | tctaaggtca | 19860 |
| agggttgaaa | tctggtgatg | gccttcctgc | tggttgggac | tctcagtaga | gggctgaggt | 19920 |
| ggtgcaggac | atcacatggt | gagggggctg | agtgtgctaa | catgttattt | taggcaggcc | 19980 |
| tcccttctta | tcaagcctct | agttccattc | ccaagataac | ccattaatcc | attgataggg | 20040 |
| gcagagctct | catgatccaa | tcacctttta | aaggccctac | ctctcaatac | tgccacagtg | 20100 |
| gaatgtaagt | ttcaacatga | gttttgaaga | agacgaatat | tcaagccata | gcttcaactt | 20160 |
| tcaataccac | caccaaaagt | atctatttag | tgaaagaaaa | aaataaaagg | gctgaactga | 20220 |
| gccacggctt | tgggttagtt | cagcataaat | tgttcgagaa | aaccatgcag | cagagcttct | 20280 |
| gtacagcaca | agtggaaaga | gtaagaatga | ggctcagaaa | ccaccatgtg | cacttgcccc | 20340 |
| atccagagag | aagtggaggg | ctgagctgct | gtcacagtga | cctttgttct | gtcagtaaca | 20400 |
| catggcctca | gcctgctgtc | cactcagccc | atcctgctga | aaccaaaggg | gaaaaaggaa | 20460 |
| tgggagaagg | atgagaatca | aacatctggt | gaggaggagg | gaggcccgtg | ctattcggat | 20520 |
| ctggtataaa | gctcccagga | attgcaaagc | tgttcttagc | acatcagctc | ggtgggcctg | 20580 |
| ggcggatgga | ggcagatgca | cacagcaaca | ccaggagcag | gggaaagtct | gccttttaag | 20640 |
| gttcatgttc | cccagcaggc | cctttcccat | tctaaacacg | tttcccacca | ttctctcatc | 20700 |
| cccagtgtgt | cgttttaccc | taactgtgtg | tgtgtgactc | acgtgaaact | gagtttcaat | 20760 |
| ctgatcacat | accacagggt | atgtttggaa | aaccttttgt | gatttaagat | ttatttaatg | 20820 |
| atttctattt | taaagtactc | ctaatcctag | tttcatggac | atttatactc | agagagttat | 20880 |
| gtcgagcctt | taaataacaa | cctcacacat | tctttctaag | ttaaattcca | atggctagcc | 20940 |
| ttgcaaatgg | tttggtgctt | ttataaaata | ctgcattaag | tcttaaagat | agatttctgg | 21000 |
| gagaaaagtt | ttgatatgtg | tctttttata | tatgtggagt | tctctctttt | gaaagaggag | 21060 |
| aaaaattaga | aattacatat | gtgtttagag | attgcttaaa | atatcactta | aagcagaaga | 21120 |
| gcacaaccat | aagtgttgaa | ttaaggtgga | gaccgcctaa | caggaaggta | tgcaactttc | 21180 |
| cctaaattcc | tccagcctgc | gctggctcca | aggaaccaag | gttagaagct | cccgagtgct | 21240 |
| acaggaacgt | ctccgccaag | gaagccatat | atgctcgccc | atctagtcca | gaggaaatca | 21300 |
| cccttggctc | ccctttctaa | ctcaaaggac | gcttttttct | cactattttg | gcagccacaa | 21360 |
| ggctgttaga | gtcaaaggaa | gtcctttgtc | ttttctggga | gagctccatc | tatgtgggga | 21420 |
| tgtgcatcca | aagcaacttt | ccagtcattg | aggctgacct | gcagttactc | accagtgcac | 21480 |
| tggtgcccat | cgccggcata | accaggcagg | caggcacagc | tgaacgtgct | gcctccatgg | 21540 |
| tgaacacacc | gggcctgccc | agcaggagca | caggtatgac | tgccatcctc | acaggggttg | 21600 |
| gcaggtgggg | tgatcactga | aaagagaaga | agataagggc | atccagagtt | taggcctgca | 21660 |
| aaggatgagc | ctcactcctc | atggcatgat | ccacagacca | acagcaacag | accagggaat | 21720 |
| gcaggttctc | agaccctacc | caaggtccag | tgattcagaa | tctggatttt | aataagattc | 21780 |
| ctagatggtt | tgtatgctat | taaagttcaa | gtactggtat | gaatttgctg | ttaaaatact | 21840 |
| aagcacaaaa | acccatcacc | taagcagtcc | tcaattaaag | tttataacat | tccagagcaa | 21900 |
| ttgctacaca | cacacacaca | cacacacaca | cacacacacg | cagtttctcc | tgtcactttt | 21960 |
| accctcatcc | cactgaatta | aagagtgcat | tttctttctt | tttttttttt | ttttgagatg | 22020 |
| gagtctcact | ctgtcgccag | gctggagtac | agtggcgtga | tctcggctca | ctgcaacctc | 22080 |
| caactccctg | gttcaagtga | ttctcctgcc | tcagcctcct | gagtagctgg | gattacaggc | 22140 |
| gtgagccacc | atgcctggct | aaagagtgca | ttttctttac | tctcatccat | ctttatcaat | 22200 |
| gggatgaatt | gggatagttc | ttttgatcca | tttttaggtc | tgtagtattt | tctggaaata | 22260 |
| cagaagaggt | aatttgttac | agaaggagct | ctttagccag | tcaagtagga | catgcatcag | 22320 |
| tgaattataa | atccatctct | tctaatttga | aaaatacttt | aggctctatc | taaagagctg | 22380 |
| gaggtgttta | ctagtgaagt | tcatcatctt | gttacatgac | tgggccagaa | atcccaactg | 22440 |
| aggtgggcat | ccttcaataa | ctcaacacca | aaaatatcta | tttagggaaa | aacagtgaaa | 22500 |
| ataaacaggg | ctgaaactga | gccatagctt | tgagagattt | tgttgttttt | ttttttgttt | 22560 |
| ttttgagaca | gagtctcact | ctattaccta | ggctggaatg | cactagcatg | ctgtcagttc | 22620 |
| actgcaacct | ctgcctcctg | cgctcaagca | atcctcctat | ctcagcctcg | tgagtagctg | 22680 |
| ggactacagg | cgtgtgccac | cacacctggc | taatgttttg | tattgtttgt | agagacgggg | 22740 |
| ttttgctatg | ttgcccaggc | tggtctcgaa | cttctgaact | caagcgatcc | acctgcctca | 22800 |
| gccttccaaa | gcgatgatta | caggcgtgag | ccaccacatc | cagccagctt | tgggttattt | 22860 |
| tgatgtaaac | tgtcttctta | agaaaaccat | atagcagagc | ctctgaatag | cagaagtcaa | 22920 |
| aacactgaca | gattaaagag | caagattaaa | gagcaccatt | ttggccacac | aggaaaatga | 22980 |
| ttttggaact | cacagatgca | agtatgccgg | tcatctgcaa | actcataacc | actccggcac | 23040 |
| tcacacctgt | agcttccagg | caagttgata | catacagagt | tggggccaca | gcgatgaaag | 23100 |
| ccagttgcac | attcattttc | atctaagaag | aaatgagaag | agaaaaattc | tgcttaattc | 23160 |
| aagggtccca | gaagtggagg | gtctaaaaac | tcaatgtttt | ctcactagta | tgatgcttca | 23220 |
| atgggacaaa | tggttggcag | atgtagaatt | tttaaaaatc | agatatgaat | gcatgttgca | 23280 |
| gataatcaga | aatagtaaaa | atttcatgcc | attcaagtaa | acaccacagg | aagataaatt | 23340 |
| tttaagtgaa | tattctaaga | attctttaac | tgttttaaaa | agaatggata | cttggaagaa | 23400 |
| aaccaacaat | tttttttaat | atcaagaaat | ttctaccgtt | gccttttggg | gatataagac | 23460 |
| acacaatgtc | tgtctaaaat | ttaaagaaaa | tcagaaaatg | tgctttttgg | taagtctaag | 23520 |
| aactacttac | tacaaaagct | caaaattaca | aaacttaact | caaaacatcc | aggaactgca | 23580 |
| acagatgttt | tcttttcaca | gccccggtgc | aaaatccact | ccactccaaa | ccatcttttc | 23640 |
| aaacataaac | agctatgctt | taatttttaa | ataactatat | tcagattaac | agctgctaaa | 23700 |
| atcattgaca | atggaggttg | taaaggtgac | agatacttgt | cttctacaga | cataaggctg | 23760 |
| gggagggcag | aggaaaaaca | agggctacaa | gaaggagaca | cgagaacatg | gctcttaccc | 23820 |
| acacagttcc | gtccatctcc | ctggtaccca | gatgcgcact | cacaggtgta | atctacacct | 23880 |
| gtccctggat | ggcaccgtgc | tgttgtgtca | cacatgtggc | tcccatcata | gcaaggattc | 23940 |
| cccggagtgg | ggtctgaatc | ctctgcatga | gtagagggga | aataaaagca | caatctggct | 24000 |
| attggtaagc | aaatgtcacg | gagatagagg | agtcctcgga | actgactgca | tgtctgtgtt | 24060 |
| ttgtgacaca | ccggaagacc | ttactaaatt | atccatatct | gaagctaggg | tggattttct | 24120 |
| gcacttcagt | tttaaactcc | agcatggctt | cctattgcct | accctataga | caactagtac | 24180 |
| ctttacttgc | tttcagtagg | tttgcatcca | gggaataaaa | acgcccaaaa | catctgttta | 24240 |
| aaagtgcttt | tgctctatta | tttcctgtga | ttcttgggat | gattctatgt | ggtgagttgt | 24300 |
| tttccccatt | atgtgtaatt | ttcacagata | aggaaatcaa | gtgagacatg | ggaaaaattg | 24360 |
| cccaagctca | caaagtcagg | aagtggcaca | gcaaagactc | agactccaac | ttcctggttc | 24420 |
| taaatctgtt | gatctcttac | taaaattccc | aatttttata | ggtaaatcca | attaatttta | 24480 |
| aaaatcttgt | tttttaaaga | tttctaagac | taaacaggaa | tgactttccc | agtatttact | 24540 |
| cttcaggagt | agccttacca | aggatccatc | tactcactag | gtagcgatta | cttaacgact | 24600 |
| gaaatctccc | ctgagagaaa | tctatttggc | tgagcatggt | ggctcatgcc | tgtaatccca | 24660 |
| gcactttggg | aggctgaggt | aggaggatag | tttgaagcca | ggagttcaag | accagcctgg | 24720 |
| gcaatatgcc | aagaccttat | ctcgagagaa | agaaaagaaa | gaaagaaaga | aagagaaaga | 24780 |
| aagaaagaaa | agaaagaaag | aaagaaagaa | agaaagaaag | aaagaaagaa | agaaagaaag | 24840 |
| gaaggaaggg | aaagaaagaa | agaaagaaag | aaagaaagag | aaagaaaaag | aaagaaagag | 24900 |
| aaagaaagag | aaagaaaaag | agaaagaaag | aaagaaaaag | aaaagaaaga | aaaggaaaag | 24960 |
| aaaagaaaga | aaattagccg | agcatggtgg | cccatgcttg | tagtcccagc | tactccagag | 25020 |
| ggtgaggcag | gaggatgact | tgagcccagg | agcttaagct | tgcagtgagc | catgattgtg | 25080 |
| ccactgcact | ccagcctagg | tgacagaaca | ggaccatgtc | ttaaaatata | tatatttatt | 25140 |
| tctacataat | agtcaatgct | cattgagcat | ttatgtgtga | ggcattgttc | taaacagata | 25200 |
| ggaggatgcc | tgattacatc | cttctaacta | catggtgagg | aaaatactat | ctccacttct | 25260 |
| gctgcatcac | agagaggcca | aataacctgc | ccagggttac | acagtcagaa | gaggtggatg | 25320 |
| gagctggaat | aaaagtctag | cagtctggtt | ccatagctca | tgctttccac | cgctttgctg | 25380 |
| caaccctcac | tgaaggaact | tcatgaagtt | atctagtcta | agccaatggc | acagaggtat | 25440 |
| tggaaggagt | gtcagatgct | caggtagaat | gaatctatca | actctggcac | ttctgtgtac | 25500 |
| ttctgagtct | gccatgtaaa | atggaaatag | gaacatgtac | atctgccaat | cctgctaaag | 25560 |
| aaaattaaga | aaacccaaca | gaacatattt | gaaaggcaat | tccccattgg | caggtctacc | 25620 |
| cagtctctac | tgactgcact | ccttctgcct | tctcagtggg | gccactgttc | atcattattt | 25680 |
| gaggttctag | ctcaacctca | gtccctagaa | ttaactctaa | tcttagactc | aaaagatttc | 25740 |
| cagagcccca | gctaagcctg | tcaggttcta | cattgagcca | tctctgctac | aaagggagat | 25800 |
| ccatctaagt | tgtaataact | caggcttctg | cctcatactt | ctgtgcctga | ggccctgtct | 25860 |
| tgatagccag | cttagcatcc | tctgagtccc | atcctgctca | ccagttctca | ctactcacag | 25920 |
| acaccagcat | catggcatct | gtaggcagac | actggcagta | ccacccattt | taccaccttc | 25980 |
| tcctggattc | tctatcattt | cactctttct | gacccgtgtc | tcccacccca | ctttgtacag | 26040 |
| aatcttctat | taaaatggag | aaataacaac | aggagagaat | caggttcttg | tcttatactg | 26100 |
| agccttcaat | gaggacatga | ccctgttgtt | ctccattgta | acagatgctg | actcaggaac | 26160 |
| ctggtctata | ctctcaagtt | aacttggcaa | ccacaacccc | actgctatgg | ggggagactt | 26220 |
| gcccagagtg | actcctaatc | tagatcccaa | gtgctgactg | ctgcagctga | cctcatagac | 26280 |
| tggactcgct | cccaagtcct | tcagggtgtc | tggcattctc | ccccgtgcct | gccctactgc | 26340 |
| ccacgttatg | gctcttccag | tgccctgtca | tagcctgctg | tgagctggga | tgccttggct | 26400 |
| gagacagcct | tgttttctgg | taaactcttt | ccatcccact | ttataagaaa | caccacagtg | 26460 |
| cattcttgta | gtggaatatt | cccgtattat | tgattcccgt | attattggct | gaaaataagg | 26520 |
| agggctatgg | gtttatcctc | agttcaccac | aatgttgtcg | gctggagtaa | cagaatcttt | 26580 |
| gccaaaacaa | aggaccaata | aataattcca | gcacttttta | aacctgatga | actatgtcag | 26640 |
| cataagccta | gctccctgaa | gcatgcagac | atccttggag | gtccgttcct | ctggatctct | 26700 |
| tggctgacag | gaaagaggca | ccgcactggg | gttgtggttg | ccgagttaac | ttgagcctag | 26760 |
| accaggttcc | tgagtcagtc | ctcaataggt | tataactaag | gacaaggtca | tgtcttcatc | 26820 |
| cattaggacc | caatgtaaga | caagaacgtg | atcatctccc | atcatttctc | cattttagga | 26880 |
| agattctgta | caaaatgcct | ggcatacccc | accaaagcag | gaagtaattg | tcaacatgtt | 26940 |
| ttgatttata | aatactaatg | cttagccttc | ttattcagta | ctttccagca | tcaaaatttc | 27000 |
| accatggcac | attccagcaa | agattagtgt | gttcacatgg | acatcaagag | cttaccaccc | 27060 |
| tgctaggcat | taaaagaaaa | aaaaaaaatc | tcagctcaaa | agttgacttg | caaacttgga | 27120 |
| caggatatat | tatcctacct | aaaattgaga | agaaagtgtc | cctaattact | caatactgat | 27180 |
| ttaaaatgta | tgtcactgac | aggattagtt | ccataatttg | caatccccag | tgcaaaatga | 27240 |
| aaatgtggag | cccatggttc | aaagcaaagc | gttaaaccaa | gcatggggct | cttctgagca | 27300 |
| cagggcccca | ttcactgaac | ttggcttccc | aaaacatttt | taaaatacat | ttctaaaacc | 27360 |
| atttcacgac | tcattctcta | tgacttgtaa | gcttttctcc | tattatagta | agtaaagaac | 27420 |
| acagtatcct | tcttaagaca | ttttctattt | cgccctcacc | ctcttaaact | gcctgactct | 27480 |
| cttaacagag | gatctggaac | taatttactc | atttaatcaa | ggggaagagc | ggttttcctt | 27540 |
| aaaggaacag | tcccttcagc | ttcactgcac | agccctctgc | ccagagcctc | ctcaactcgc | 27600 |
| caaaaggacc | cgtgaagtgg | aaggacagga | agcatttttg | gatcgtgttt | tatgacccca | 27660 |
| caaaacaacc | aaccaaccaa | caacaacaaa | aaacagctga | ctcacactta | aagcttaaag | 27720 |
| ctcaaacaac | tggtgggggg | tggcactgct | ggttgagaag | tcaccgaggg | acagaggctc | 27780 |
| caattactca | ctgggaaaga | caccccagtt | ctgcatcctc | gccctgggaa | gtcgcaccac | 27840 |
| acccacatca | ggtgtggaca | gctgcgctgt | ttcacatgaa | ttccaacaac | tatcgttccc | 27900 |
| tctggaaaaa | ctgagcagtt | cttaggaaga | tactgctgtg | taccaagagg | ggaaagaaaa | 27960 |
| aacttttggg | tgtttctttt | ttgtttttct | ttttttccct | cctattcagg | tagaatgaac | 28020 |
| agtactgctg | tgtgtggatg | aagtagggtt | caaacacaaa | gggtttaaaa | gacacacatt | 28080 |
| gcctctttca | aagtttggtc | taaattatga | ggcagtcatg | aggccactgt | ggaacagcag | 28140 |
| agccatgagg | ggtaagggtg | tgcgtactgg | agccagtgcc | tgagtttgag | tcccagctca | 28200 |
| cccaggtcac | ttactatgac | caggcaaatt | acttcatttc | tatacctcag | tctcatcatc | 28260 |
| tgtaaaatgg | gataacaaat | aaccccaccc | ccaccccacc | attgggtaga | ggtcattgtg | 28320 |
| gggattaaat | gagttaattc | atgtaaggtg | cttagaacag | tacttggcac | caagtaaacc | 28380 |
| atcctttgat | attattattt | gactggggtg | ggagggaaaa | tcataaaatt | ctaaagaaaa | 28440 |
| caagcaactt | ggtacctgcc | agtttgcttt | ctctgatcct | ttcttactct | catttggtat | 28500 |
| gtctctagga | aaaatatacc | cagagacaca | tctttttcct | aaatggttcc | agaatctgaa | 28560 |
| tgggactcca | tagcctggga | aatttcacta | acagttgctt | ttacagatca | gatgcctaca | 28620 |
| cccaacacag | ggctgacctg | gacatttaaa | ggtaaagacc | attctcccca | gtaggtttaa | 28680 |
| atctctcttt | gagaaatcta | ctacagcaat | ctttcccttt | ctaatttaca | tgttcctcta | 28740 |
| aaatcaactc | acattttcat | gtgttcattt | gctaccttac | aatccaatat | ctgggcacct | 28800 |
| aatgcctaca | ttcccaacca | agtttcttaa | aaccaaagca | cctcgggatt | ctcattacaa | 28860 |
| gtcactttcg | catcctagaa | aagagtctag | tccttgtacc | ttaatcttgt | catattgctg | 28920 |
| aagcaacact | tctttattga | gcctgctgag | gcttaaggag | ccctcagctc | caataacaaa | 28980 |
| gcacagaacc | gcgtatcact | tacagattca | cctcactgag | gagctaactc | tgggctcaac | 29040 |
| agtttcaata | cagtggctga | attgaggaag | atggtttttt | tctcctggtg | tggctcttac | 29100 |
| attctagggg | aaaacatgac | ctggttttca | gaattaaaat | gtccccttga | tctgacaaag | 29160 |
| ccacttccca | tctgtagagt | tccctgactt | cacaagctca | tcctctggct | tgcttgaaac | 29200 |
| attttttaat | aattaagacc | ttgcttttta | ggatggtttt | acatttagag | aaaaatggag | 29280 |
| cagaaagtac | gagctcccat | atccccccac | ccaaaatgtg | cagtttcccc | tattatcaac | 29340 |
| attgtgcatt | agtgtggtgc | atttgttatc | cccaatcaat | actgacacat | tattaactaa | 29400 |
| tgcccatagt | ctgcagtagg | gctcactctt | ggtgttggac | agttctatag | actgacaaac | 29460 |
| gcataatggc | ctataaccaa | cattacaata | tcatacagaa | tagtttcact | gccctaaaaa | 29520 |
| tcccatgttc | cacctatgca | tccttctcct | tcttcctcct | gaaccactgg | caaccactga | 29580 |
| tctttttact | gtctctatag | tgtaaccttt | tccagaatgt | catggttaga | atgattctgt | 29640 |
| atgtagcctt | ttcagactag | ctgctttcac | agagcaacat | gcttttaagt | ttcctgcatg | 29700 |
| tctttctgtg | acatgatatg | cctagaacat | tttaaaatca | gtcttcacag | gtgaggctaa | 29760 |
| tggtcattaa | aatttgagaa | ccactgctta | ctttgagacg | gggtcttgtt | atgtggccaa | 29820 |
| ctctggcctc | aaactcctgg | gctcaggtga | tcctcccacc | tcagcctcca | gagtagctgg | 29880 |
| gattacaggt | gtaagccact | gtacccgatg | agaatggttt | tagagttttg | tttctactac | 29940 |
| tatttgcttc | catttatttt | tattttttga | gacggagtct | cactctgttg | cacaggctgg | 30000 |
| aatgcgatgg | cacaatcttg | gctcactgca | acctccgcct | cctgggttca | atcagttctc | 30060 |
| ctgtttcagc | ctcctgagta | gctgggacta | cagacacatg | ccaccacacc | tggctaaatt | 30120 |
| tttttgtgta | tatatatata | tatatatata | tgttttattt | tgtagagaca | gggtttcacc | 30180 |
| atgttggcca | ggctggtctt | gaactcctga | cctcaggtga | tccacccacc | tcagcctccc | 30240 |
| aaagtgctgg | gattacaggc | ataagccagt | atgcctggcc | ttcagttgag | aagtaattca | 30300 |
| cataccatac | aattcatccg | tttaaagtac | ataattaaac | aatttcgtct | attcagtgtt | 30360 |
| atccaaccgt | caccacaatc | aatttcagaa | cattttcatc | acccccaaaa | ttaaccctgt | 30420 |
| accctttagc | tattaacgcc | ctatgctctc | attatctctt | agccctgggc | aaccatgaat | 30480 |
| atgctttcta | tgtatgtgga | tttgcctatt | ttggatattt | cacataattg | taaccattca | 30540 |
| gtatgctgtc | ctttgtcact | gactaccttc | acttaaggtt | ttcagagttc | atccatgttg | 30600 |
| cagcatgtat | ccgtacttca | ttccttttta | tggttgaata | atattccagt | atatgaatat | 30660 |
| attttttggt | gtccatcaac | tgatgggcat | gtatgttgct | tccacctttt | ggctattatg | 30720 |
| aatactgcca | ctatgaatat | acacgaataa | gtttttgtgg | ggatctatgt | tttcatttct | 30780 |
| tttgggtata | tagaaaggag | tagaattgct | ccattaaatg | gtaactttgt | gtttcacttt | 30840 |
| gagaaaccac | tattttgttt | ttcacagtag | ccacatcatt | ttataatcac | accagcaacg | 30900 |
| tatgagggtt | ccgaattttc | catgttctcg | ccaacacttg | ttattacccg | ttttttgatt | 30960 |
| atactcgacc | acatgatgta | aagtagtatc | tcattgtggt | tttgatttgc | atttccctga | 31020 |
| tgactaatga | tgctgagcat | ctttacacat | gcccattggg | tatctgtata | tctttttgga | 31080 |
| gagaggtcct | tgcagagctt | ttgcccatct | tttagttagc | ttgttttttt | attgagttgt | 31140 |
| aagatacttc | gtatactcta | gaacaaatat | cacatgattt | aaaatactgt | ctcccattct | 31200 |
| atggctgtgt | tttcactttg | ttaataatgt | cctctgaagc | acaaactgtt | aaaattttga | 31260 |
| tgacacccag | tttattttgc | tgttgcttgt | gcttttagtg | tcgtacctaa | gaaaccatta | 31320 |
| gcaaattcaa | agtcacaaaa | atttacccat | atgttttctt | ctaaaagttt | taactcttac | 31380 |
| acttaggtct | gttggcccat | tttgagttgg | atatgctgtg | aggtaaaagg | ttcaacttca | 31440 |
| ttcttttgta | tgtggtcact | cagtggtccc | agcaccatag | ttaacaatag | tttaaaagac | 31500 |
| tattctttct | ccattgaatt | gttttggcac | tgtgtttttt | gttttgtttt | gtcttttaac | 31560 |
| catcagaatt | ctattattat | tatactttaa | gttttagggt | acatgtttac | aacgtgcagg | 31620 |
| tttgttacat | atgtgtacat | gtgccatgtt | ggtgagctgc | acccattaac | tcgtcattta | 31680 |
| gcattaggta | tatctcctaa | tgctatccct | ccccactccc | ggcactgttg | ttgaaaaagc | 31740 |
| aattgacttt | taatgtgagt | atttctgggt | tcccacttct | gttcctttga | tctttacatg | 31800 |
| tatccttacg | ccaggaccac | actgtcttga | ttactctaga | tttcgagttt | gagaaataca | 31860 |
| agtctgactt | tattcttttc | caagattgtt | ttggctattc | tgtgttcttt | gaatttccat | 31920 |
| ataaattcta | gaatcagctt | gccaatttct | tcaaagagcc | agctgagatt | ctgataggga | 31980 |
| ttatgttgaa | ttaatagatc | aatttgggaa | gtattcacat | cttaacagta | ttaagtcttc | 32040 |
| caatccatga | atgtggaaca | tcttcccatt | taactgggtc | ttaaattttt | ttccaaaaat | 32100 |
| gttttgtggg | atgcaatgta | caagtactgc | acttcttttt | aaaaatttat | ctctttttga | 32160 |
| aacaatttct | taatctttag | tttgctcatt | gctactatag | agaaatacaa | tttggtgtac | 32220 |
| tgatcttgca | tatcttataa | cattgcgaac | ttgttcttta | gttctaatag | tgttttagtg | 32280 |
| aattcctgag | attttctaca | caaaatgtca | tctgagacta | attttacttt | gtccttttcc | 32340 |
| aatttagatg | gcttttattt | tgtgttcttg | cctaatcacc | ctggctagaa | attctagtac | 32400 |
| tgaattgaat | aaaagtggca | acagcagaca | tccttgtctt | gttcctaagc | ttcagggaaa | 32460 |
| agcatccagt | ctttcaccat | taagtataat | gttctgggaa | tcatggaggg | ggaggttata | 32520 |
| catcaagttc | tatgatgttt | ggtttttcca | gatactagct | ctttgctcca | ccttagtttt | 32580 |
| ggatgaaatt | cttctcatct | gttttagcta | aaatgtcact | ttcttgggga | catcttttct | 32640 |
| gaatcttcaa | actagactca | cagtgccctg | ctctcatcac | acttgtaaca | aatacttgtc | 32700 |
| agcatcactc | tccctcctaa | ttccagtctg | ccttgttcac | aggtatattc | ctgattggca | 32760 |
| cgcacagttg | ggtattgcta | taaaccagct | tcacatacta | tgctctattt | cctgactttg | 32820 |
| gcagggacac | tgttccctgc | cctaaaactc | ttttctgcct | ctgtttccaa | ctcaagggca | 32880 |
| aggatctgct | ctattcatcc | ttatacctag | cacataacac | agcatggcRc | taggtaaccc | 32940 |
| ttagtaacct | ctgtgaatca | ttctaactct | acttaaaagg | atgtcatttt | tacccaacaa | 33000 |
| caaaaaagga | aaccttacct | ttgaccgggc | caatttgatt | ggtcacagca | aatctaagca | 33060 |
| ctctttcttc | gtcattatac | aaggcaaaga | cccggtccac | gttcagctgc | tgggtggtgg | 33120 |
| ggaaggacgg | gtgtctgggg | gcgtgcctgc | acacctggta | agtgatgttc | tggtggatgc | 33180 |
| ggtaggacca | tgtttggttg | attgcaccaa | aagtcagaga | gtagtctctg | gaacttgtag | 33240 |
| aggtcacagc | tgcaacaaac | acagtggtaa | ttttttaaaa | atattaaata | cagagatttt | 33300 |
| catgtgaggt | ggatttttct | gcagtttttt | tcatctaatt | cttggagtgt | gttcccttgg | 33360 |
| gaacctcctg | agtttatatg | aaaaaatgtg | taaggaaatc | ccaaaaacct | gagtggatgc | 33420 |
| aaagccacag | tgctgagagg | gagaaggcac | tacaattgtc | catgttatta | tacacaaaga | 33480 |
| ataagtgtta | ctaagggcca | gccatgccaa | cagtgctctc | tacatactgc | ctcatttatt | 33540 |
| cctcacaaca | gcctggtgag | gtgggcacta | aatcatgtga | tcagaggctc | acaaaggtta | 33600 |
| agtagcctaa | gatgcaatgc | tgccacatgg | tagagctgga | actgaaactc | acattggttt | 33660 |
| gactctacac | cctaaatttt | aaaccaatcc | atcttctaca | tttcacagaa | gtgacgactt | 33720 |
| ccaacacctc | gcacttcagt | aaatctgaag | gattccagag | aagggaaagg | gttgtctctc | 33780 |
| accatctgag | tctaggagtc | aagctcaggt | cagtgatccg | gacactgacc | attttgttca | 33840 |
| cagcccaggt | ctctgcttcc | ccttgtcttc | cctacatctc | ctgccaggct | gccagctctc | 33900 |
| agcataggag | cagcaggggt | cagaggaaag | gcacaactca | atccggattg | ctgtgctgca | 33960 |
| caacagcagc | tgtgatggag | gcagcatcct | gtgcgcaggt | agtactttca | cagaacacat | 34020 |
| cacgcttcgc | acacattgat | cacataatat | agaaaaagca | tttttagttc | catgtgaaag | 34080 |
| atgagaaaat | acattctcag | atattaagag | gcctttccct | atagatatta | aatggcaagt | 34140 |
| ccaggatata | aattcttatc | ttttgactcc | aaatctagca | ttagttttat | aataaattat | 34200 |
| agcatctctg | tgttttttgg | ttttgttttt | gagatggagt | cttgctttgt | cgcccaggct | 34260 |
| ggagtacaat | ggcacaatct | cggctcactg | caacctccac | ttcccaggtt | cagatgattc | 34320 |
| tcctgcctca | gcctcccaag | tagctgggat | cgcaggagcg | tgccaccaca | ccctgctaat | 34380 |
| ttttgtattt | ttagtagaaa | caggttttca | ccatgttggc | caggctggtc | ttgaactcct | 34440 |
| gacctcaggt | gatccaccca | ccttggcctc | ccaaagtggt | gggattacag | gcatgagcca | 34500 |
| ttgcacctgg | cctctttctg | tgattttttt | tttttttaaa | gcagcctgaa | ctcatcagct | 34560 |
| gaaacaaggt | tgagataaat | ataaattcca | ataatcacta | tttttccatg | ttgcagaaag | 34620 |
| taggatggtt | aaaaactgta | tataattata | ccatccctac | ccaccccatg | gacaatgttg | 34680 |
| ctattgattt | tattatttga | aaatactatg | tagggatata | gatattctta | tgtatagcag | 34740 |
| ccattctgct | cacttacacg | atatatttca | tggtaacaat | aagagtaata | acataataga | 34800 |
| gctcatttta | tgttccatgc | actgttctaa | ggacatgcca | tgtaagtcat | ttaagccttg | 34860 |
| tatctatctt | gtgagatggg | cataatcccc | attttacaga | tgtgaagact | ggttatacat | 34920 |
| actggagtcg | gagtagtggt | acagctcctt | gtagggagag | atgtgggctg | tgaaatttgc | 34980 |
| tgRgacgtaa | ggcacctggc | cttgaatgtt | ggtcttaatg | ctcaggtagt | tctctgggtc | 35040 |
| aagtccctca | gcagtttgag | tgatacgaac | cgtctcctct | cccgggtaga | atgtaacttc | 35100 |
| catgtcatgg | gtaaaggcag | cacctggaga | tgaaaaacat | tcagcacagg | gatgaaaaga | 35160 |
| ggtcttgctt | aaacagttac | cagtatatac | tgcccgctcc | caatttctac | tgctgttgga | 35220 |
| gatcgtgcct | aaggagatta | tctgatatct | gagagtaagc | actatcagct | gttgattcca | 35280 |
| taagttccca | aacttttcca | ttgaagaata | tggtttcatc | ctgccttacc | tcacccagct | 35340 |
| tctctttgaa | caagcttgta | ttctgtgaga | aatactcttg | ggaacgtcct | tgacttttct | 35400 |
| ctaaggttgc | tagacttagc | aaaaaacaaa | caaacaaata | aacctaacaa | caaaaaaaca | 35460 |
| aaaacaaata | aactaccacc | taagacattc | agggttttgt | ttgaatttca | gataagtatg | 35520 |
| tctcatgaaa | tatgtgggta | atacttatac | tagaaaattg | tttatctgaa | atacaaattt | 35580 |
| atccaggtgt | gccatttttt | atctactttt | gacccaaggg | ctttattcaa | attttgatta | 35640 |
| aagcaagaac | ctattaacag | ctaccgagct | ctgcagttaa | agatggagaa | agaacttctc | 35700 |
| acaaaactat | ttttaatgca | ataaagataa | aacctctagc | aaacaagttt | tcaggttgga | 35760 |
| aggtgagaat | ccaaaaagta | tcttctaaat | ccttgttgtc | aacgggtggc | ctttccagca | 35820 |
| acactggtac | ctcacctcct | agcttggtag | aaaagcaaaa | tcacggaccc | caccccaggc | 35880 |
| ctgctgaacc | agaatttaca | ttttgacaag | acctccaagt | gatgtgtgtg | caggttcaag | 35940 |
| tctggggtgc | accggcctat | actattatag | gttgtggagc | tgggaggaga | agagcacccc | 36000 |
| ttccccatga | agtaaaatca | tccccaggtg | ccccaagtac | catttttgca | tttgacagat | 36060 |
| ctgtgcataa | tttaaaaagc | ctgacaaata | tgctgcaaca | aattggggta | agaggacaca | 36120 |
| aatgttctga | atggaatcat | cctgtttctc | agaacagctg | ggagaacatt | tagacgaaat | 36180 |
| cactgccttt | caaaaatcag | tgatgtgagg | tcaaaccatt | cccaacaacc | tgtggccaga | 36240 |
| aaactacaca | catgtggctc | tagtacatta | aacaagtgat | tcctctgtca | ctgcgtaaag | 36300 |
| gcctaaagac | agtgccccaa | ggagcaccac | tgccaaagca | atgctgacta | ccggccttct | 36360 |
| cagaggccct | actcacctgc | gaggctgaag | ccgttctcag | agccaggttt | ttctaaagca | 36420 |
| aagagccagc | caaacaggcc | tccaattggt | gtgaggggga | ggagggcctg | ggctgctggc | 36480 |
| tgtgggatgt | ggctgatggc | cgtgtaggct | ctgccatcat | tgcccacgat | atacgcatgc | 36540 |
| aggtccacat | cagtgaagtg | cacgggtgta | tggcccacgt | ggaggtggcc | actcactttc | 36600 |
| ccattcactc | ggtgaggtgc | ccctaaaaga | cagcaaatcc | agttaggctt | ggacgtcatc | 36660 |
| ctggctagag | atgggtgtac | ccaggtataa | ttattccact | gacaacttcc | aaaactcact | 36720 |
| ctttagcaag | tcactttaat | gatttcctaa | ccaaccaatt | atccatctat | gaaacagtcc | 36780 |
| tgcctccagt | ctgttttctc | ccctcctcct | tctactccac | tacactttgg | ctctaatgag | 36840 |
| ctggcagaag | gaagcatttc | tggataatcc | tcaaattggg | aatcagccca | aaccagttca | 36900 |
| ttcctcacac | atttattgag | cacctgcaag | ttgatagcac | tctgatttaa | gtagtggaga | 36960 |
| tgtttggtcc | ataaagcaga | gctcacagtg | gtagctggaa | acaggtaagc | agcaggaata | 37020 |
| gacacacagg | agttcctggc | cccagtcaat | taccttcagg | cagacagtgc | ttcccatttc | 37080 |
| cataaaactt | ggattggcag | tggcagcaga | agccagtggc | atagtccgtg | cagaaggcat | 37140 |
| gccgggagca | ttgtctgtgg | ttgtgttcac | aggtttcctt | gttggcagca | ttatacgtga | 37200 |
| agactgaaaa | ataaaacaaa | cttgccttaa | aaggactaaa | tgattccaat | gtcactgctg | 37260 |
| catcggggac | acaacatctg | ctccatagaa | gcagtttaga | tcataacaaa | cagtttttga | 37320 |
| tgtttaagag | accggcataa | cattcaaccc | aagggaatta | atctgctttg | tctgctggca | 37380 |
| agaagaattg | cttactgctc | ccggctgggt | cccattgcag | agttggtgaa | ctaagatgag | 37440 |
| actgcacccg | tgggtttgag | tgcagtaaaa | tgaaagcaaa | ccagtaagta | catgttaggt | 37500 |
| tgaacttcat | gaaaccacta | tttttgtggg | tcaaaaaggg | ctaaatatca | gcacttttat | 37560 |
| atggttcaac | ctaaacacat | cctgagagga | agacataata | gagaagctaa | caaatgtctt | 37620 |
| atgtctaacc | atccttcttt | tagggaacac | tgaacttaaa | aaagataata | gctggtctcg | 37680 |
| ctaccaagat | tgttttgagg | gaagggtctt | aaagaacaca | cgcacctacc | tcaaagaaaa | 37740 |
| ggcatgggga | gaaggggaca | gaggagggca | aaacaacagg | gattcaagct | attaaaatgc | 37800 |
| cctatgccct | tggcagggtt | taactatgat | gggtatggta | caagcatgat | ttccacgcag | 37860 |
| tagcagacag | cttgggcccc | actcgcagat | cttccaagaa | attatggagg | actaggataa | 37920 |
| attcagtgct | ctggcagctg | tttcttccat | ttcctactta | ttctgcccat | taaaagaaga | 37980 |
| gggccatctg | tgctgcaaag | aaatggggaa | actggagcaa | atgccatagt | tcataaagga | 38040 |
| aatagccaag | ggggtagggt | gtggcggggg | ttgtggatgg | acaggagggt | gctttctccg | 38100 |
| gggcactagg | aaatatgcca | atcccaagca | tggccaccag | gaggccaagc | ggctgcacac | 38160 |
| acctctgcct | ccacatcccc | aggaggtgga | gttggctgga | gactccagga | gcatgctgtt | 38220 |
| gagcagggat | ggcatggaat | tctagtgtga | gacacggatg | ctggcatgaa | ggaggacacc | 38280 |
| caccctactc | ttaggaaaaa | ccaagtggta | gttgtgttcc | aggtctatct | ggaaacgagg | 38340 |
| agacaggagg | ggaggaagaa | atccaaagat | caatctttac | ccagtattcc | cagggagaac | 38400 |
| agaaagtaaa | ggagctaatg | agacatagca | ttataggtac | aatgtcacca | agataaagac | 38460 |
| ctccatcagc | aaataaaaat | ataacaaagt | taacacttca | atcgcgctta | acatatacaa | 38520 |
| gctttacatt | taacaacctt | tttttttttt | tttttttttg | aaacggagtc | tcgctctgtc | 38580 |
| gcccaggctg | gagtgcagtg | gcacgatcac | agctcactgc | aagctccacc | tcccaggttc | 38640 |
| acgccattct | cttgcctcag | cctcctgagt | agctgggact | acaggcaccc | accaccacgc | 38700 |
| ctgactaatt | ttttgtattt | tttagtagag | acagggtttc | accatgttag | ccaggatggt | 38760 |
| cctgatctcc | tgacctcgtg | atccacctgc | ctcggcctcc | caaagtgctg | ggataacagg | 38820 |
| cgtgagccac | cccgcccggc | caacaactct | taatttgcac | aacaacccta | ggtagcaggg | 38880 |
| actttttttt | gttgctgttg | ttgagacagg | gttttgctct | gtcacccaag | ctggagagca | 38940 |
| gtggtgccat | ctcggctcac | tgaaagctcg | acttcccagg | ctcaagtgat | cctcctattt | 39000 |
| cagccttttg | agtagccgca | gccacaagca | tgcatcacac | ctgcctactt | tattttttgg | 39060 |
| tagggtgggt | tggggggagg | ggtctccctg | cgttgcccag | gttggtctcg | gtctcgaact | 39120 |
| cctgggctca | agtgatcttc | ccacctaggc | ctcccaaaat | gctgggatta | caggtatgag | 39180 |
| ccaccactct | ggccagggac | aattttttaa | cccgtttgca | gataaagctg | aggcacagta | 39240 |
| gactaaagca | cctgaagtcg | cataggcagt | gagtggcaaa | ggtgggaacc | gaacctagga | 39300 |
| catctagctg | gagaatctta | actgctacta | tccacgaccc | cttcaatggg | atccaagtac | 39360 |
| ggagatcatg | agcactagat | taggggtcac | cacatgtctg | gggttaatca | gcacagactt | 39420 |
| caagtataat | tcgagtcata | ctttataaac | tgggtttaat | ggtagaagca | agcagggaag | 39480 |
| attatcgaga | tgactattac | agcatgttga | aaaacagcac | aggtaagaaa | tgagagtagc | 39540 |
| agcagcggca | ttagcttttg | aacagtactg | gagtgctgcc | tgcagatcct | tgggaaagca | 39600 |
| gcaagctgag | ggttggcagg | acagacagtg | taggttcaag | tggaaaatct | gattagaaca | 39660 |
| tagtaggaat | gaagttatga | ctcaagattt | agtatagggc | cttgatgact | tggctaagaa | 39720 |
| attccagttt | gataaaaata | gaaaattagg | catgactcaa | ctttatttaa | acaagaaagt | 39780 |
| gacaagctta | acttaaacag | aaaagtgata | taaaactcaa | ggcagaaaaa | aaaaaaaaaa | 39840 |
| aaacccagga | tggatcatgg | taaaatagat | ggctgtgaag | aaaactgtat | cactgtctat | 39900 |
| tagtttaaga | aagggaaaaa | aacatttaga | gtaaaaggaa | atgaatgtaa | cttggatgtg | 39960 |
| tcaaaatctc | aggtttctcc | agctcagaaa | ttcacaacta | agtgtaagga | agcagctaca | 40020 |
| ggagggactc | acacactcac | cagtgtgctc | atggcttaag | ggccatcagg | actttctccc | 40080 |
| agagctcaag | gaatgagggg | ctgcagaaag | cgccaagctc | taggactcaa | cctcagccct | 40140 |
| ccctcggggg | ctttgtggaa | actatgcaaa | ctgtctttca | gcttcctcat | ctctaaaata | 40200 |
| agtagaacac | ctcttcctca | cccctgcatt | gccagaagtc | gcatttcttc | ttaatgtact | 40260 |
| tctgaacacc | aagtaaaaag | gcaatctgct | ataggtctaa | agaacagcca | tttggctggg | 40320 |
| cccggcagct | cacacctgta | atcccagcac | tttgggaggc | cgaggcaggc | ggatcacgag | 40380 |
| gtcaggagac | tgagaccatc | ctggctaaca | cggtgaaacc | ccgtctctac | taaaaataca | 40440 |
| aaaaattagc | caggcgtggt | ggtgggcgcc | tgtagtccca | gctactcggg | aggctgaggc | 40500 |
| aggagaatgg | cgtgaatctg | gcaggcggag | cttgcagtga | gccgagatcg | caccactgca | 40560 |
| ctccagcctg | ggcgacagag | agagactcca | tctcaaaaaa | aaaaaaaaaa | aaaagccgtt | 40620 |
| ttcataatat | tcagataaaa | atcaatccaa | actttacaag | ctgttctgcc | cacaaattaa | 40680 |
| ggaaataaca | ggcttggatt | gtatcttatt | tcagttattt | tgtgaagcct | tattctgcaa | 40740 |
| cccaattctt | attctgaggc | tgaattatga | aacaaggaga | gagaagatgg | aggagagaca | 40800 |
| gtctagatat | ggagagttaa | actaggtgga | gtcatagctt | ttatctaagt | aaaaagaggc | 40860 |
| agggcgatgc | agccagcaac | ttacaagtgc | aactgtgcaa | aggtttataa | atagattgga | 40920 |
| agctgtacta | catttttcta | ttacagaaaa | agaagttata | acaatgagca | ggctcccatg | 40980 |
| aaaggctatt | atgagactta | aactgtaatt | acaatgggct | aactagttac | tgttaagcta | 41040 |
| acctgggagt | ctttttaaac | agttttgtgg | aatattgtat | ttttaggatg | gtacttactg | 41100 |
| agccaagaac | acacactgtc | tctttcagct | tcataacagc | cacatgaagg | aaagaagatg | 41160 |
| gatcagcccc | taatccgggg | gaaggcaagc | ctccatttat | aatccctctt | ccacgcctgc | 41220 |
| cttcataccc | aagtgcacac | acaggtttgc | acacttacca | tttacaatgg | cagcttgtta | 41280 |
| ggtgaaaggt | aaatgcttct | tacaattttt | gttcattcat | tcactaattc | aacaagtatt | 41340 |
| tactgagcat | tcactataga | cctgtaccta | gtctagaaac | tgaggacatg | gcagggagca | 41400 |
| aaagagacaa | aaattcacat | cctgaagctt | acattctccc | cagcagcagt | caataaacaa | 41460 |
| gtaaattact | tagtggggtg | gatgtgtaag | agctgtgcaa | agaaatacag | cagagaaaga | 41520 |
| caagagggtg | ggagggggca | ggtgtacatg | gctcttttta | atagcatggt | cagggaagac | 41580 |
| ctcaggagaa | ggtgactgag | cagaggccag | gaggaggtga | gggagtgaac | cccacagatg | 41640 |
| agggcaggcg | tggtgctgtg | tcaggcagga | ggggtgtgtg | ggcatgtggg | agggatggtg | 41700 |
| agagaagagg | tggcctgggt | cattcatgca | gggtctcaga | gatcacagga | aggatttgag | 41760 |
| ctttcatctg | agtgagatgg | gaagcactag | agggtttcga | gcagaagagt | aacatgctcc | 41820 |
| gactacagta | aatcagacag | gagctggtcc | cttggtggta | agaagtgctg | gtacagggga | 41880 |
| taagaagggc | ccaggtctgg | gatattctga | aggtggaacc | gagagggttt | gctggtagac | 41940 |
| tggatgtggg | atgatgttca | gtcctgatgt | gggaaaccaa | aagggtggag | ctgccagtta | 42000 |
| ctgagatagg | gagactgagg | aggagcaggt | ttgtggggtt | cagttctgac | aacagcatga | 42060 |
| ggcatcaagg | aaattcacca | aggggtgaat | aacaacttac | accctcagca | cagcaccatg | 42120 |
| cagtccttca | cacggagagc | tgcatttctc | ccggtggatt | ccagagacca | ctgcatcagg | 42180 |
| atcacctgag | atttctgtta | aaaatgcaaa | ttctgagggc | ccaccttaga | cctaccagtt | 42240 |
| ccactctctg | ggtgcatcct | agcaatctac | actttagcca | ccccgccagt | gattctcaca | 42300 |
| ccaattggtg | ttgatagaag | gcttgggata | caacaagcct | tggatttctg | agctaggtgt | 42360 |
| tgctcatcaa | tgtcgttaac | aaatttaatc | tgaccaattg | gcagctcctg | gggtgggcag | 42420 |
| aatgttagga | aatttctcgt | gctcatttct | ggccacctga | gaagctgagc | tcagtgctta | 42480 |
| cggcccattt | gggcatccat | ccacaagaat | acaacgtgaa | gattctttag | tgaaagtaat | 42540 |
| acagacgcac | agcagccaga | tagcccttcc | ctgaagaagg | gggagacctg | gagctagaac | 42600 |
| ggtgctgagc | ccacgtgcag | ctatttatcc | agggcgagaa | aagccagacc | tccggccaac | 42660 |
| gtgactgctc | aaatttctat | gggaattgca | gtgaaattca | aagaggctga | acttcttgaa | 42720 |
| ggctgggaat | caagatctgg | atcccagcta | gatgagatgt | gcaataaacc | cacccgagga | 42780 |
| aaagtggcac | tttttctttt | cggtggtcat | tttcatgtcc | ctatcatggc | tttgagtgta | 42840 |
| ttttcatgca | cttcttccta | ttctagttag | aagatatctc | cattttataa | atgagaaaat | 42900 |
| tgaggcctac | agagatgaaa | caatcttctg | gctctaaatc | ccatcaaatt | gagggggcat | 42960 |
| cagaatcact | gaagtgcaga | ggaatatggc | agggtcagac | aagagggtga | gagagggcag | 43020 |
| gcacacctgg | ctctttgaaa | tagtatggtc | agggaagacc | tcgggagaag | gagacatttg | 43080 |
| ggcagaggcc | tggaggtgtt | gaaggagtga | ggtggcttaa | aaatgcagtt | tctggttcag | 43140 |
| aaggtagagg | aggcgcttgg | aaatcagtgt | ttttaaccag | gctcccagct | gagtgagagg | 43200 |
| aaggtggatg | agtgcccaca | ctttgagaaa | tacagtttct | agtccatggc | ctcctctggg | 43260 |
| acccacacta | tgccaggaag | ctcctgccac | ggagagtcag | accataaaga | agtttgctga | 43320 |
| cacctcaggg | cacagcagga | gggtggaggc | tggccaggtt | tgaatgaact | cattttgtca | 43380 |
| tggatttgtt | aactgaggtt | tgatagttgc | taaaggattc | ttaacatgaa | gattttttaa | 43440 |
| atctcttttt | tttctattta | atgttttttt | tgtttgtttt | taacttttct | catgtcctta | 43500 |
| atagaaagaa | aaatggggtg | gggagatatc | agaggaaaga | aagaaatgcc | ctgccaaagg | 43560 |
| acttgagact | tgtcctaact | aggagttcgt | aacaaaaatg | gaaaattctc | ctctgcactt | 43620 |
| gttctgagcc | atctttcaat | cttcattttc | ccagaaacac | ttaagcacct | gtctgcagcc | 43680 |
| cttggccggc | ctgcccttct | tcacagaagg | ccctgggcca | gacacagggg | aggttccatg | 43740 |
| gtgtcgactg | ggttcactgg | gccaacacag | agggacacag | gaggcttcgt | gcacagggct | 43800 |
| gcttcaggac | agctggagca | actactaaca | atttttactg | ctacctcaaa | aaaaaaaaaa | 43860 |
| gtcattttca | ccgcaggaaa | aattcttcta | agttttccca | tctctcagaa | tgacagacta | 43920 |
| gttagaaaaa | ccatatccaa | ctttataaca | aacagtggaa | tgctttttcg | tccagttttc | 43980 |
| atgtttgttt | aattttgatg | tgtgtggcat | gtaatgatgc | tggtggcagt | gatcacgacc | 44040 |
| ctgggggtgg | ccttttacaa | ctgctggcat | gacactaaca | ggctctctcc | acttctggtg | 44100 |
| aagggagagg | ggccctacca | tggaatctcc | tcatataggt | gtcttcacag | gcatcggggg | 44160 |
| gcggcggctc | ctggtcacag | accctctgca | acctggaggt | gcacccaggg | gatggtcttt | 44220 |
| ggcacctgga | gggttgcact | ggcagcagaa | cctgcttaaa | ttcttctcag | ggtggaagtt | 44280 |
| gagcttgcaa | cacactcctg | cagcctttca | caaggccaaa | agtggagtgg | gactacatga | 44340 |
| ggtttctgaa | agccaggtgt | ccttcctcct | aactcatcat | gcagattgag | attaaattaa | 44400 |
| gctgcgtata | gcactgcctc | agcaggctta | agaataacta | agacccctat | cagtaaacct | 44460 |
| cattcctttc | tactcacaac | acaggcagtc | ccatggtccc | aggcagctga | agccctcact | 44520 |
| ggcaactctc | tcaccccact | gccttaatca | acctttgtct | ctgtctctct | ccctcctttc | 44580 |
| ctccctcctc | attcccatct | ctgcaactcc | gctcttgtca | cttctctgtt | cataatgttt | 44640 |
| tcacacattc | tccctctcag | actttgaccc | caggcctggc | tcaagcccct | ccttcctcac | 44700 |
| aaagacttcc | ccaacagcag | tgacactcct | ggacagtgcc | tttgactctc | tgtcacccta | 44760 |
| cagctttctc | taatttcttc | atgactttgt | tgcagtcttt | gcccttgcta | gacaagtcac | 44820 |
| taagtatttc | caagctcagt | ttccatctct | gtaaaatggg | aaggatgatt | gtacctaccc | 44880 |
| tacaggacaa | cttttttttt | cagacaatat | ttctctctgt | tgctcaggct | atgcagtggt | 44940 |
| acaatcatgg | ctcactgtag | cctggaactc | ttgggctcaa | gtgatcctcc | cacctcagtc | 45000 |
| tcccgagtag | ctaggaccac | aggtgagcac | caccacaccc | ggctaatttt | taaaattgtt | 45060 |
| tatagaggca | aggtctccct | atgttgccca | ggctggtctc | gaactcccca | cagggcaatt | 45120 |
| ctgaggacta | actgtgataa | tgcaatctaa | tgccaagttc | tcactttaga | gcagcaggct | 45180 |
| gcacttagca | aatgccagcc | aaaacttcca | ctagcaccga | gtcagagaca | ggtacacggc | 45240 |
| aggtgcttgg | gtaacttttt | gacttgacaa | caaaacgacc | agagctccct | ggaaacaaga | 45300 |
| ctcctcagag | aaagcatgct | tgcacatcca | tctactgctg | ttctgtggcc | taggaagaca | 45360 |
| gagctcgagt | gtggccagga | cattcctgcc | tggtgtagat | atgatctgaa | aatctaaatt | 45420 |
| tctgaacaaa | aagaaaggtc | tgtaaaatga | gtccctaagg | ggaaaaaaat | gtaaaaagga | 45480 |
| gtgatatttt | ctgggccttt | aactaaatct | taatttcttg | gtttagcaag | atgactaccc | 45540 |
| taaagcttca | aagggaaact | aaaatgtatt | gggcagggtg | cttcccaagc | tcaatttcag | 45600 |
| ttcttgcaag | tatatgacga | ggtcttcatt | aactctgttt | tagagctgag | gagagccaag | 45660 |
| ggtcaaacag | actacatgtc | ttgccccaaa | ccacatagca | gagctgggag | tcaaggtcaa | 45720 |
| gtccaaccag | caccaaaacc | atgtgctttc | tattccatca | gcagaaaaag | ccaggcagat | 45780 |
| gccacatggc | tgggtactta | gtaccggaca | atgttctgtg | tctctgccac | tcccacccca | 45840 |
| cagcttcccg | gaggaagccc | acacttacca | gacagacctc | tgagcctcca | atgttttatg | 45900 |
| tgaaagaaac | agtcactgca | tcccctcaaa | agtatttaac | aattgcatgc | ttctatactt | 45960 |
| catacatccc | cgtaaattct | tttaaaacaa | aaactggatt | ttttttgtct | aagtttcaaa | 46020 |
| cacttctaga | tttcttcctc | caaaatgcgt | attaaccagc | tttttcacag | gcgttttttg | 46080 |
| ccattaccag | gatgtctata | gtagactgca | tccggcagct | gataaatatc | atagttcatg | 46140 |
| ccccacaagg | gttatagctg | tgcctctgtt | ctactctatg | gctggttcct | gaagcctcct | 46200 |
| gcactttact | ccagtgatct | caagctctca | gagcccaaag | cagggtgtgc | agtcccttct | 46260 |
| taacccttta | atacatgact | ccatagatct | gctttttaat | attcccacgc | tcttcccagg | 46320 |
| gaaggaaatc | acaacccaca | aatgagaagg | ccattcagga | atgcgtcttt | acctcaaatc | 46380 |
| cctttgtgaa | aatctttgat | acttggcctt | aaaatgtatt | tagaggaaac | tcatactgta | 46440 |
| aattctggag | attctcagaa | ggatctgaag | gttctaaaga | cttccaataa | aatttacatc | 46500 |
| ttcaacttca | tggaaatgtg | gtcccttagg | cacaaattaa | tgtgaaactc | tttaataagc | 46560 |
| acattcttca | ttctctaccc | atcctccacc | ccattcccac | tgttttaaat | cttctaaaat | 46620 |
| tagtaacagc | agccactatt | tattaagaac | cagtgagtgc | agacacgaag | ccaagaactt | 46680 |
| cacatacgct | gtctcattga | atcctcccaa | gtaattcctg | agaaatcaga | ggcttatggg | 46740 |
| ttacataagt | tacttcaggt | cacccagcaa | gcttggcagc | caggattcaa | actcgaagcc | 46800 |
| gacaccaatg | tactccctcg | aatccttaca | caatgccaca | ttagtcagaa | aatattttct | 46860 |
| tccctcctga | aattagtacc | tctccatgca | atagcctgca | gataatacca | gagcagaacc | 46920 |
| cagaaggccc | tctctgctag | gtcagccatg | tttttgccac | aacagtgaca | ggaaacagaa | 46980 |
| aaggtggaaa | aacatggaga | ttcagaacaa | agcagtcacc | tctgacctct | ttcttcctat | 47040 |
| cagaggtcag | atgagcagtc | cctggaacaa | acaatctgga | gtcagtttcc | cattcacatc | 47100 |
| tttccatgtc | tgatggggaa | gaaacagctg | gcatgacaag | tctcaacaag | gactagaaga | 47160 |
| ctgacataga | ccccatgtcc | atgataatta | accaccgcct | gctttcgcca | tctccctcct | 47220 |
| cgcctctcag | tccgatcagt | gggtcatgaa | atgaggacaa | ggctccaggg | aggttcatgc | 47280 |
| cacagctgca | tagccatgaa | ccagaaatga | caacagacac | aggtgtttag | tcattttctt | 47340 |
| tccctgtggg | ctctcctctc | ccacacatca | caagcatatc | tccccaacca | agggaagcgc | 47400 |
| taggcttgtc | tcacaacaga | gtatgaatct | ggcatgttca | tctgagccag | cacaactcac | 47460 |
| cactttaaaa | agcacaaacc | cgtccatcac | tggaatgtgt | atgaagccgc | aaaatgtttt | 47520 |
| acctttgctg | aactttcaaa | ctgaaatagt | tcattacact | tgagagcgta | gctggcataa | 47580 |
| caaacccagt | tttcccccac | tcacttcatg | aggcccatat | ttttaaaaaa | cattattaat | 47640 |
| gatcaagaga | ttccgaacag | aatctgcctg | ccaagattta | tttggcaaag | gaaatttact | 47700 |
| ctatctcttg | aaacacaggg | attaaaaatt | agatccaatc | ttctttttgc | taaacttttc | 47760 |
| acctacccac | ttaagagaaa | aatcacaacg | ctatgcaaaa | ccagcatggt | taagatgaaa | 47820 |
| ccttagcacc | cagttttcta | atgcactcac | cctcggtgtt | ggaacctatg | ttgtcttcca | 47880 |
| gtcccacctc | atacgtccct | cgacttaagg | gtgtagtgtg | acctgaagca | gggtaacttc | 47940 |
| gaagaacaat | ttcttcttca | ggatgagctg | ggggaacatc | catttccgaa | ggcactggcc | 48000 |
| ctccatctgg | gtagggctgg | atgcttccgt | tttcggggta | cggtggtggg | gtttcccagg | 48060 |
| aaggagccag | tgaatctctg | tctacctctg | gtggagctgg | gcttctggtc | tctctctcat | 48120 |
| cccagggctc | aacttggcct | tttaaatctg | ggccccctac | ctctcccaga | gatgttcctt | 48180 |
| ctttggtgtg | aggatccaag | gtggaagatt | ctgtttcagg | atagaatggc | caataagtag | 48240 |
| gtgttggatg | caagggggag | gacagatcag | aatctggagg | ggagatccta | ccctctaaag | 48300 |
| gctttgtatc | cactttggat | tggaaggaaa | catcaatgct | gctgtggcca | ttcaatgcct | 48360 |
| cctctggttc | acccggaagg | tattcagctt | cctcctcatt | cacatcatag | taatccaaat | 48420 |
| tgtcctcatt | atagtcactt | tccagggctg | tagcatggct | gaaggaacgt | cccaggggaa | 48480 |
| cagaagagtg | ggcagcggaa | aggtctccaa | ctgcagctgg | cctgacattg | tccaacgggg | 48540 |
| aagtgctgcc | gatatggaaa | gcccacactc | cagggatccc | caggttgctt | agtctaaata | 48600 |
| aaaaggaaac | agtcattgta | acaaatgtaa | caaaagtgtc | cattcaccca | ccttcctaga | 48660 |
| aggtgaacaa | cttattttaa | aacggaaaga | cagctgggca | tggtggctca | cacttataat | 48720 |
| cccagcactt | tgcaaggcca | aagtaggagg | atgacttaag | cccaggagtt | tgagaccagt | 48780 |
| ctaggcaaca | tagtgagacc | ctgcctctac | aaaacccttt | tctaaaaaac | ttagccaggc | 48840 |
| atggtgccac | atgcctgtgc | ttctagctac | tcagtaggct | gaggcaggag | ggtcacttga | 48900 |
| gcccagcagt | tccaggttac | agtgggcttt | ggttgcaacc | actgcacccc | agcctgtgtg | 48960 |
| gcagagtgag | accctgtctc | aaaaaaacaa | aaaacctgaa | aggctattaa | gatcaggttc | 49020 |
| agttttcact | ggtatataaa | accaaataaa | agcagactta | catggtaact | aaagggatca | 49080 |
| tactggactt | ttgatgtgaa | ctacctttta | tttaacatta | tgtttcaaag | ctgtatgcca | 49140 |
| cagaatgcca | gatcatcaaa | ttatcaatct | gccccataat | ccaaatggtt | tatctaaaat | 49200 |
| ctggctattt | gaaaataaac | aaggaatgta | ggaatgtttc | atccttcctc | tacttgtctg | 49260 |
| gaaacataac | acattttaaa | aatgtatgct | taaaactcta | ccaaatactg | acacccaaat | 49320 |
| gactacgaca | tactttcatg | aattggtgat | gccccttccc | tgtgccaagt | gtttcacatt | 49380 |
| cttcaataca | atctgcaaat | agaaatgatc | atattcaatt | ttatagaggg | gaaaataggc | 49440 |
| tttaaaaggc | taactgatct | tctgaaggtc | acataacaaa | caattggatc | cgaattcagt | 49500 |
| taacaccaaa | gcccatgctc | tactcatcta | tgttatatta | tgacagaaaa | ccataccaca | 49560 |
| cactgcagtc | aataagcatc | tgaattatca | ttactctaca | gaatattaaa | gacatttcat | 49620 |
| tgtttattca | cttgaactga | ttaattccct | aatccacaaa | gaatggtgag | tcaatgtaaa | 49680 |
| aaaattcaaa | taaatacaaa | tgccaaaacc | taaaaaattt | gtttttgcac | tgaaaaagtt | 49740 |
| aaggagcaaa | taagaaagta | gcttagaagt | cttttaaaga | cttctggaaa | cagctctctc | 49800 |
| gttttctcca | tgagaaaaga | acatgaactc | cattgagttt | ccaaatatcc | caataaccca | 49860 |
| ttattttaga | gtcaatggta | taccctaaag | agaacaacta | catatgattt | ctaaaaatat | 49920 |
| ttattttaga | gtcaatggta | taccctaaag | agaacaacta | catatgattt | ctaaaaatat | 49920 |
| gggaaaactt | gagtgaacaa | gagtctggtt | aggttgaata | tgccagtttg | tatactcctc | 49980 |
| tgatatttgt | acaaaacact | atgctcacat | tctcccaagc | aatggaattt | cctcaaaagt | 50040 |
| cagtcaatga | ggaataacag | tccatgccta | ataagataat | aaaaacttca | ctaaattgta | 50100 |
| taagaacaaa | agattttcct | tcatttgggt | gggtactcat | catgttcatg | tggatttcta | 50160 |
| tagtaacaat | atttttcagt | acaatttgaa | aatgtctttc | tcttgtgtgt | gtttgtgtgt | 50220 |
| gtgtgtgtgt | atgcatgtac | atatatgtat | atgctgtggt | tactgttgtg | gtaaatttag | 50280 |
| ggaaaaaagc | atagaaagaa | aaactgattt | tgtgtgcaat | atgctataca | tatttgttaa | 50340 |
| gttcataaga | attaatgttg | gtgtttattg | gcaagcaaat | aaggaaaact | aaagacagta | 50400 |
| ggagaaaatc | ctgaagttct | gaggatattg | agtttattat | cacattagag | gtggtgcaac | 50460 |
| tatgaaattt | cttcttgaag | ttagaaacag | aaactataaa | atcttcaaac | agtggaaaaa | 50520 |
| tacagattat | ggccaataaa | agctaaataa | aataaccttc | taattatata | taaatcacta | 50580 |
| tctgggtttt | gtcattatgc | ataatggaaa | agtaaccaat | ttgagtttat | tttaaaatcc | 50640 |
| aaggtcattt | ggaaagctta | gtaatagaaa | gaacttaatt | tatttctaca | aaggaaaatt | 50700 |
| gggttacaaa | atgggatgac | aatgccttcc | tccctgggtt | gttaggagaa | ttaagtgaga | 50760 |
| aaaccacatt | aaaatgccta | gcactgacca | ggtacggtgg | ttcatgcctg | aaatcccagc | 50820 |
| actttgggaa | atcaaggtgg | gcggatcacc | tgaggttagg | agttcaagac | cagcctggcc | 50880 |
| aacatggtga | aaccccacta | taaatacaaa | aaaaatagtc | aggtgtgatg | gtgtgtgcct | 50940 |
| gtaatcccag | ctactctgga | ggctaaggca | gaagaaaagc | ttgaacccag | gaggcagagg | 51000 |
| ttgcagtgaa | ccgaggtcat | gccactgcac | tccagcctgg | atgacagaac | gagactccat | 51060 |
| ctcaaaaaaa | taaataaata | aaatgcctat | cactggccct | aaacatcata | aatacttatt | 51120 |
| aactactgtc | ttgtcttcca | gaagatagaa | aagtatgatt | tcaaaatata | cacgtgtgaa | 51180 |
| ttcgtattta | aatttgcaga | ggaagagtat | gacaaacatt | atgaactata | taatttatga | 51240 |
| cctttgagct | cacagttgtg | agacaaattt | gaggaagaaa | acaattgact | aggaaaataa | 51300 |
| ctttactctt | aattacttat | ttatttagag | gtagggtctc | cctctgtcac | ccaggctgga | 51360 |
| gcatagtggt | gtgattgatc | atggctcact | gtagcctcaa | ccccctgggc | tcaagtgatc | 51420 |
| ctcccacttc | agcctcccaa | atagctgggt | ctacgggtgc | acaccaccac | acccagctaa | 51480 |
| ttttttaaat | ttttagtaga | gattaggtct | cgctatgttg | cccaggctgg | ccttgaatgc | 51540 |
| ctgagctcaa | gcaatcctcc | caccttggcc | tcccaaagcg | ctgggattag | aaccaccttg | 51600 |
| cccagcctaa | ctttaccctt | aagagatgca | tactaaggca | tttagcaatg | aactggcatg | 51660 |
| atgtttgtga | cttactttaa | aacatttaaa | caggccgggt | atggtggctt | acacccataa | 51720 |
| tccctgcact | ttgggaggct | gaggcaggtg | gatcacctga | ggtcaggagt | tttagaccag | 51780 |
| cctggccaac | atgatgaaac | ccagtctcta | cgaaaaatac | aaaaattagc | cgggtgtggt | 51840 |
| ggtgggcgcc | tgtaatccca | gctactggga | ggctgaggca | ggagaattgc | ttgaacccag | 51900 |
| gaggcagagg | ttgcaatgag | cacaagatca | caccactaca | ctccagcctg | ggcgacaaga | 51960 |
| gcgaacctcc | gtctcaaaaa | aaaaaaaaaa | aaaaaaaaag | aaaagaaaaa | aatgtaaaca | 52020 |
| aacaaaaaaa | tgaatatggc | aaaatgttta | caatgtataa | atctaggtga | tgggcatttg | 52080 |
| ggtgttcatc | tagttattct | ctccactttt | ctgtatattt | ttgaaatttt | aaataatgaa | 52140 |
| aagtgaaata | gaagattata | ttgaaggaca | tttctataaa | tttgatgtac | agaagttctt | 52200 |
| cctaagaata | acaacaagat | ccaaaatcag | aaaggaaaag | accagtaact | ttgacaatct | 52260 |
| gaaatgtaca | acttttaaat | catacaccct | ttctcagcaa | ttccactttt | aagaatttgt | 52320 |
| tttagggaga | aacgagaatg | tgagcaatgt | ttattatgct | gtttactgca | atgatattta | 52380 |
| taacaagaaa | aatcagaaat | acatgaaatg | ttcattaata | agctgtcttt | atacatatat | 52440 |
| gacataacgt | tttgtagcct | taaaatggta | gtatagacag | acatctgact | aaaagacccc | 52500 |
| tataagaagt | gattaatgtg | aaaacaccac | ttacatagca | aaagaaccat | tttggtttta | 52560 |
| aaagttttgt | atatacctaa | attctagaag | gaccattaac | acttgttatc | acttgaagta | 52620 |
| gtgatcatgg | ataactttta | cttttatttc | acattattct | gcattgcttg | tactttatta | 52680 |
| tttgtattaa | gcatggatta | caataaagaa | aaatacgtga | gtcatttggc | cccatatctt | 52740 |
| actctgcttt | cgccaagcaa | ttcagagatt | tctgggaagt | atcaaagata | aaaatgaggg | 52800 |
| aaggcatgaa | agggggtaag | tggaaatgct | gtctgtgtgt | ttgtgtgtca | cacgtgattg | 52860 |
| tggatgttag | ttccaagaga | cctttatggc | tctgctcagg | gaacacaact | gctttttccc | 52920 |
| acaggtcttt | tggccccact | gtctcctgtc | tcctgtgtag | atgccagcac | ttacccaggg | 52980 |
| ctctgtaatt | gttgatctat | ttgtctatca | accctaatag | ggtacaagct | gctgatgtgc | 53040 |
| aggaccagtg | tcttagaaat | ctttgtattc | catgtgcttg | gccattgtga | cctctctata | 53100 |
| ataataaatc | tatggaataa | taaatctatg | agctcccaag | tagaagagag | ggtggaacca | 53160 |
| gaggagggag | agtaaggaac | tgagtttgct | ccagagtcca | gactaagcgg | cagcctagat | 53220 |
| ggagccctaa | agatgcccaa | tgtggatcat | aaggatggga | aaggaaacaa | ggactcagca | 53280 |
| taagataaaa | taagtagttc | agcactccag | acttgagctt | ggattttggc | tttgccactt | 53340 |
| aactttgaga | tcataggcaa | tttacttaac | ttccctaagc | tcctctgtaa | aactgggaaa | 53400 |
| atagtagtac | taacctacaa | ttcttgtgag | ttttaattaa | gataacatct | ggaaagtgtt | 53460 |
| tagcatgatg | cttggcaagt | agtaagtttt | caataaatgt | cagctctgag | taagcaaacc | 53520 |
| tggctatagt | ggtctctttc | tggtagccga | gcagagtgga | tggcatgcta | aaagtgtaag | 53580 |
| gtgtttctat | acctacccag | gattgattta | atgaagtcaa | gaagcagtta | aaggaggcca | 53640 |
| ggaagtaacc | aatgtattct | atctggcttc | tatattcagg | aaacgaataa | aagcgagggc | 53700 |
| acccctacct | cccctcacac | accccccaaa | aaaccctgaa | gtcaataaat | atcctactgg | 53760 |
| cactatctgt | actggcaaaa | tgcagaagag | cttattattg | cactagctaa | gtaagccctc | 53820 |
| tggatcattc | attcattcac | ccatctattt | attaaatgcc | cattatgggc | agacactgat | 53880 |
| gagtctaagg | aatgggttca | attcatttta | ggtaatgaag | ccagagagac | atcctcagac | 53940 |
| ttctggaaac | ctataaatct | tagttatttt | tcctgcaaat | tacattgctc | aatttaatgt | 54000 |
| ttaacagtat | ctgtccaaaa | gactaaatca | caatgtttcc | tcctgtgaaa | tgattaatct | 54060 |
| ggaaaacctc | aaactgggca | gtttatgact | ggatattttt | atgtggttcc | aagagcaatg | 54120 |
| tttttcatag | caagcctcag | tggctctaca | aatacacaca | catactgttc | tgtttccagt | 54180 |
| atctataccc | aatagagcag | gaaaaataaa | ggacctttta | tgttttggtg | aacttgatcc | 54240 |
| ttccagggtt | gaatctagtt | ccttggtttt | gacttgatgc | cttacttaga | agccaccaat | 54300 |
| atattatgta | atggtcttat | ggtcacttgt | gttcaggtac | ttcctaaaag | tccttatatt | 54360 |
| caaataggaa | agggcttcag | ctcaatgtta | cttactgata | gagatttttc | acagactgtt | 54420 |
| cagtgctagt | caagctgaaa | tatggtcctt | ctgacttcag | atcatcagcc | tcccctcggc | 54480 |
| agaagcccac | ccgagctgga | agctgaagct | ggacattgta | agactctttg | gggcgggttc | 56540 |
| caaggaactg | caggccgttg | gcaggataaa | gaaagagggc | gtagctatca | gacccatcag | 54600 |
| atgccaaaac | tgcctggaaa | gtgttcagct | gtgaggaaaa | aaaaaaaaaa | agagagagag | 54660 |
| agagagagaa | acatcctgta | aaaaacaaat | aaaagaagaa | agaaacagaa | tgcaagcaaa | 54720 |
| gcatcataac | gtgcaaagaa | cttccccttt | ttcaggattc | ctccaaattg | tttttaaaca | 54780 |
| taaccttctt | taaaatttcc | tcttcaagtt | aacaatataa | tttcataagc | aatattattt | 54840 |
| attattgcaa | ataccatttg | tcaatttaga | ttaatagttg | aaatacgcat | tggtaaaaca | 54900 |
| taggttaaaa | aatatatact | ttgtaaggtt | tcaaacttct | ctcatgtaaa | ctctttcaga | 54960 |
| gctaagagca | atcagcaaaa | ttaacttgtc | catcctgcag | tcactgccct | agtttataac | 55020 |
| cctaggcact | cagcatctct | catttgcatc | attcattcat | tcattcattc | atccatttat | 55080 |
| taaatgacca | ctatgggcag | gcactgtgtt | aggcactagt | tggtcaaaag | gcctccttac | 55140 |
| tgtctcacag | catcactctc | tttcctcttc | catctaccct | ttcatacgag | catcagggtt | 55200 |
| atcctcattt | cacagatgag | aaaaagacac | aggggctaac | aggtcttcct | caggtcaggt | 55260 |
| ggcaagtatg | tggtggtggg | ccagccagct | ctagagccca | caccctagga | caccctaacc | 55320 |
| cctcctcaac | atggcattca | aggtgagttg | taacctggct | gcatacgtcc | ctcccaggct | 55380 |
| cagttccact | gcatctttgt | cagactattt | aatagtcccc | atttgtacca | tgtccttgga | 55440 |
| tgcttctaca | ctttgttcgt | ggttccctgt | gcctagaaag | cctcctaacc | ttcaaatgct | 55500 |
| gccactcaga | ggtgtttgtg | attccctata | gaataatttc | tgttctctga | acctctgtaa | 55560 |
| cacctttatg | ggcatctcta | gtgagcattc | acctcattat | gcttggttag | ttgtttatat | 55620 |
| gtttgtaaac | accattcatt | tattcattca | tacactaatt | caaacagcca | agtgtctact | 55680 |
| taagcaccag | acgttatgct | tgatgctgag | gatgtgaagg | agcaaaagac | aggctttgcc | 55740 |
| caggagagtc | tcacggtgta | ggactgggaa | acagccatgg | ttgaaatggt | aggagagtca | 55800 |
| ctggagaagg | tggtagaggc | caagagccgg | ggtgtgtgtc | caggatctca | gggtggaggt | 55860 |
| tgggaggtgt | cacagaggca | gctggtgaat | gttataaaca | ctgcaggagc | caacacagac | 55920 |
| cagagaggga | ccaggagcct | gaatcagctg | ccaattcaac | acccaggtcc | ttataggatc | 55980 |
| actccttaac | attctgttta | ccctcatatt | ttcaacaaga | tgagattttt | gcctttacct | 56040 |
| tcattcagcg | accgctcctc | ctaaagatct | gtgaacagtg | cttgattctc | aaaaagctgt | 56100 |
| tatgtttagg | cttctgaaac | tgtctctctc | tcactaaatc | acactacttt | tacaatttag | 56160 |
| taaggaactg | ggaaggtatt | tttaaaacac | aacttacttc | acaaataatg | tgactcttag | 56220 |
| aaatattatt | tactggtaag | tatcattacc | tttccattct | gctagacaga | caaactcggg | 56280 |
| cacagagaag | aacatcacat | ttcactgaag | gaaacagcaa | ggaaggaatg | ggtccttccc | 56340 |
| tttgttctaa | gtctactccc | acagttgttc | tagctctttt | ctaatgttaa | tttttccttt | 56400 |
| caatgtttaa | tttcaaagta | aggttttgag | gttttgctaa | taggataaag | aaatatagat | 56460 |
| gtaacacact | tttcaagaga | aataaaaata | caatcagcag | ttcttttagt | tctctaaccc | 56520 |
| agaaaaaaca | acatcttgct | ttgctttttt | cattcattca | attcacaaag | taacaggtgc | 56580 |
| ctagtttatt | tgttattgtt | ccagttgttg | attgttcatc | agcaaagctt | gacttttcca | 56640 |
| aaaacaaaaa | gcaagaaatg | tttttagagc | ttctcaatac | taggaagcca | caactactgc | 56700 |
| caaatataaa | caaaggctgt | tctgcctcat | gtctatccat | aagatagtcg | acatcatcaa | 56760 |
| ctaatgtgga | ctcaataact | tcagagttat | acattagtca | gaataaagaa | aaatcaacta | 56820 |
| gggaaagaaa | agtactagag | gaaggagaaa | aaaccccatt | tcaaaaagct | aagctttcaa | 56880 |
| agctcctggc | attgatctgc | cataataaat | gtgagatagg | agagaatcct | ggttctaaat | 56940 |
| ttagagaaat | tccataagat | aaggaggttg | tagagactgt | gaagaattga | aggggatggg | 57000 |
| gaagggggga | agagagtagt | aaacagagct | gtgaagagga | gagtctgact | ttttgctgtg | 57060 |
| aactctgggc | atttagcact | ttattttatt | cattcattca | ttcaaccagc | caacaaattc | 57120 |
| atttcaatta | aaatttattc | aacatcttct | atatactgtt | cttgagatga | gcactcatta | 57180 |
| tgggtggcaa | gtatttaatt | tctttgtctc | caccaaaata | ggcaaatatc | agaggaagga | 57240 |
| aacaacttcc | ttttaagtag | aatttatgat | cccaaagtac | acgccaaaag | agtttggctg | 57300 |
| tttaaaagcc | acccacacac | taaatgatca | cttgtaaagt | tcttggtcag | ttctacaaaa | 57360 |
| ttctacatct | aattgctaaa | gagcccactt | catgaagtct | tgaagggctc | cccctctcaa | 57420 |
| aaaactcctt | gactggcttc | caaacaggaa | aggtcctcac | attcctagat | acaagctttt | 57480 |
| gcaagagccg | cacaggctct | gtctccaaag | agaagccctc | agatgggctg | ccaaacctga | 57540 |
| caaacgagtc | actggttccc | agctccccca | ggatcccaga | gaggagagag | gggcctccac | 57600 |
| agggagagga | gcagcacaga | gaggatcagg | gtgagtggct | gcaggccctg | ttatcagaaa | 57660 |
| gagagaaaag | ccagagccct | ctgcagaaac | cagagcctga | ctctccctcc | caggtacagg | 57720 |
| acatcccaat | caatgttgaa | gtgacactca | tcagagccag | gctgatgcat | gcaacataca | 57780 |
| cccctctaag | agtttctaat | tttttcaatt | catttaatta | actgcagcta | tctggacaaa | 57840 |
| aatcagaata | gtgacatcct | tttcttcaag | gggcttgcct | atagaggctt | atcaaaatga | 57900 |
| aaatgaaaat | ggaaatgaag | aattagggtg | actcttatag | gggtcacaaa | gttctctttg | 57960 |
| tcatcttcct | caggaccaca | tgtcctctag | agctctagga | tcattgggtc | tggcctccag | 58020 |
| tgtttgctgt | gaacactaac | ctccattcct | tagtgtagtt | cttcatctcc | cactgcctgt | 58080 |
| aggacactgt | cacatggggg | actgtccctc | atttatttta | ctgcactaaa | tcctacagat | 58140 |
| cctgccaaaa | attcaaagac | aaatgctgag | tgctatcaca | tccctaatac | tatatccttc | 58200 |
| ctgatactct | tcagcaaaac | gtcagcattt | ctaatttcca | tatatggttc | agcattaacc | 58260 |
| aatttccata | tatgtctcta | ttctcttcat | cttcacagta | atatctcaga | ctcagtctga | 58320 |
| actctcttgg | atgggctgta | gaccacccct | cttctaaacc | ttgtctccag | aagtcaaaaa | 58380 |
| gctacaataa | agtcctgctt | ttacctctca | ttttctctgt | gaaatccaac | cacctccatc | 58440 |
| aatgactatc | aacctttgaa | ccaagtcaac | atgtctcagt | ctccacccag | atggaggcct | 58500 |
| ccattgggca | gtgtcttaat | ttgtcttctg | ttgtttacaa | caaaatactt | gaaattgggt | 58560 |
| aattcacaaa | gaaaaggaat | ttacttctta | caattatgga | ggctgagaag | tccaaagttg | 58620 |
| agaggtcaca | tctggtgagg | gccttctggc | tagtggtgac | tgcagagtgc | cagggtactt | 58680 |
| acggtatcac | atggcgacaa | agctgagtgt | gctaatgtgc | tagctcaggt | ctcaattcct | 58740 |
| cttcttataa | agcaaccagg | cccactccat | gataatccag | taatccatga | atgaataaat | 58800 |
| taatccattc | ctgagggcag | agccctcatg | acccagtcac | ctttcaaagg | ccccacctct | 58860 |
| caatactgtc | acattgagga | ttaagtttca | acatgatttt | cagaggggac | aaatattcaa | 58920 |
| ccatagcggg | caaccttgca | ctcatctgtc | ttatgttact | gcctgtgact | ccttcatcca | 58980 |
| tttctctggc | ccctttgcta | ataggttgct | ctctcaacct | tctcctgggt | catgtttttg | 59040 |
| ctcagaacaa | tctctcttga | tacctcattc | tcaatatcca | ttctttttag | ccctttcaac | 59100 |
| ctgacttaaa | ttccagataa | acaaaacatt | ccccacatta | cagagatcta | tataactcag | 59160 |
| ctgtctctaa | ccccatccct | aaaatgacaa | aaacaaaaca | ctcctctttc | cccacaaaaa | 59220 |
| gcacagctaa | acattagtcc | tctatgtctc | ccatcaataa | gtatttcaaa | ggtctacacc | 59280 |
| ataacttttt | tgctctgtga | actacctatg | agagttcaag | aaatctcaga | ttaactgaat | 59340 |
| tgacaaacac | tatttgttca | catttttttt | tcagatacaa | ttacaccttg | acattcctgc | 59400 |
| aatatcaggt | gcaacatctg | gtcacacctg | acaggaatat | ccatggagaa | aaagggcacc | 59460 |
| aactgcaatt | gtttttaatg | gcaaagatgc | cattgctttg | aatcttccag | tttccagtaa | 59520 |
| ctttgcaata | taaagttagt | aagccaaact | ttaagaacgc | catgtagaac | tcagtccttc | 59580 |
| aagcaaatta | gattactgtt | tgccaaagtc | tatctccaaa | gaactattaa | aacaagaact | 59640 |
| caagtcccag | agctctttaa | aatattggaa | gaaacttcaa | tgtgggtaat | tgaaagaaca | 59700 |
| tcccctatcc | aaacagaatc | ctcctttctt | tttttttttt | tttccccttt | cttttttatt | 59760 |
| tatttattta | tttatttatt | tattttttat | tatactctaa | gttttagggt | acatgtgcac | 59820 |
| attgtgcagg | ttagttacat | atgtatacat | gtgccatgct | ggtgcgctgc | acccactaac | 59880 |
| gtgtcatcta | gcattaggta | tatctcccaa | tgctatccct | cccccctccc | ccaaccccac | 59940 |
| cacagtcccc | agagtgtgat | attccccttc | ctgtgtccat | gtgatctcat | tgttcaattc | 60000 |
| ccacctatga | gtgagaatat | gcagtgtttg | gttttttgtt | cttgcgatag | tttactgaga | 60060 |
| atgatggttt | ccaatttcat | ccatgtccct | acaaaggaca | tgaactcatc | attttttaag | 60120 |
| gctgcatagt | attccatggt | gtatatgtgc | cacattttct | taatccagtc | tatcattgtt | 60180 |
| ggacatttgg | gttggttcca | agtctttgct | attgtgaata | gtgccgcaat | aaacatacgt | 60240 |
| gtgcatgtgt | ctttatagca | gcatgattta | tagtcctttg | ggtatatacc | cagtaatggg | 60300 |
| atggctgggt | caaatggtat | ttctagtttt | cttaatgaaa | cattctgaag | ccaactagca | 60360 |
| aatggtaatg | tctgcaggtg | gattttaatt | agaaaagtaa | aagagagatg | gaatttacaa | 60420 |
| atctcttcta | ccctaaacct | cccccacccc | agaaaaggac | cagacaaaag | ttttaattcc | 60480 |
| aaaaaccttg | catttaaaga | gtccttatgc | aacatagaga | gtaacaggat | ggttaccaga | 60540 |
| ggctgggaag | ggtagtaggg | gtcaggggaa | ggtggacaca | gttaacgggt | accaaaaaaa | 60600 |
| aaaaaataga | aggaatgaat | aagacctacc | atttgatagc | acaacagggt | gactataacc | 60660 |
| tgtgataaca | ggttattata | gtcaataata | acttaattgt | acatttttaa | ataactaaga | 60720 |
| gtgtaattag | attgtgtgta | atacaaagga | taaatgcttg | gggggatgga | taccccattc | 60780 |
| tccaagatgt | gattattacg | tattgcatgc | ctgtaccaaa | acatcccatg | taccccataa | 60840 |
| atatatacac | ctactatgta | ccctcaaaaa | ttaaaaaata | aagagtcccc | atacaaaaga | 60900 |
| tataagtcac | agcatcctta | caagatagag | atcagtgaca | tgggcaagca | acctcacctc | 60960 |
| ttacaggggg | catgtaccat | ccttctatag | tgaagaagca | tttccgtaag | ttaaaattga | 61020 |
| gatgaatcat | atggtttcag | cccctagagt | atggtatttt | agattcaggt | ctaattccat | 61080 |
| agaaggaagc | ccttactttc | ttcaggaatg | aggcagtttg | ccatttacca | agcacgctgg | 61140 |
| ccccactcat | atccatttgg | aggacttcaa | atgttgatga | caagactaaa | gaccaggagc | 61200 |
| ctggaatttt | ttactttccg | caggtagaga | ctgtaaagca | cattgacggc | agactcctat | 61260 |
| aaactaagaa | acttgtaaac | tggtacctgg | aaggcaatta | ggcagaagtt | atcaagaact | 61320 |
| ttatcaaaag | tgcatcctct | ctgacctaac | aattgcactt | cctggaatgt | caactaagaa | 61380 |
| catgtgcaaa | gatttagctt | tttaaaaagt | gattcacgct | attatttata | ataatgaaaa | 61440 |
| accagaaaaa | gccttaatgg | cccaacactg | gttgagcata | ctgtggtaca | cccactatta | 61500 |
| tacaggataa | tagcaggata | cagtagtatg | catcattata | aatgatgtag | aaatacattt | 61560 |
| attgacattg | aaacatattt | acaatacact | attctgtgaa | aaaagcaagt | tataaaacag | 61620 |
| tatgtgcagt | gtaaactcat | cattgttaat | agacagcaat | aaagatatgc | agaagaaaaa | 61680 |
| actctgaaaa | tgtacgcaga | aaaatgttta | tcagtggtgg | gaaaatgagt | ggttgttttc | 61740 |
| ttttttaaaa | ttatctgtat | tttctgattt | tttccccttc | aatgaacata | taattagaaa | 61800 |
| tacttgtata | cttagagatg | gttctaaaaa | taagcaacca | aacagtaaat | tgccttatgt | 61860 |
| tcaaaggggc | tcccacatct | gcactcccgc | tctcatatcc | gctaagcatt | tttttcctca | 61920 |
| agggagtgac | cacctgccag | ggcctcagaa | gatgcctaga | gacaacgtct | ttcctcactc | 61980 |
| ccaagccact | ccagccctgc | tgcctccaag | gtcaggttca | gcgcaagagt | aggctcagaa | 62040 |
| acaactccga | gccagtccct | gggtcgctgc | cccagaattt | aagcccatcc | ttcaatttcc | 62100 |
| tcagcagtca | aatatccctg | gtcacttacc | tctcccgagg | gcagcgcccc | gcgcttgacc | 62160 |
| tcctcgtaag | cgcctacctg | ctcccaggtg | gccaggaagg | cgtgggtggg | ggtaaagcgc | 62220 |
| gcagagcgcg | ggaagccagc | gcgcacatag | cgggcggcca | ggcccagcac | tgcgggggag | 62280 |
| gtgtcctctc | ggtacaggac | tcggcctctg | ccgtggctcg | tgtcgatgtc | cgccagaaaa | 62340 |
| ggggcgatgg | ccgggaagtc | ggtggggaaa | tcatagtcca | catactgcgt | ttccctgggg | 62400 |
| aagtcctgag | tggagatgat | gccgttggtg | cccacctggg | agaggagagg | gacaaaaagg | 62460 |
| tgacagtcgc | tcaagcccaa | ggacgctacc | ctttcgcgca | gggagggaag | ggaactgact | 62520 |
| taggcaaaaa | gcctctctct | ccttccccac | tggccaggga | gtctggattc | agggaaacaa | 62580 |
| gcgcaacttt | tgcgactgtc | ccagctccgc | gctgcagtag | gtccaggggc | cctaaaactc | 62640 |
| gttcacctat | cttctcactc | ccctcacaac | cccggagccc | catgtcctcg | gagaggaagc | 62700 |
| ctccaggtct | gaagagggaa | caccccagct | ccgcagacgg | gcacagcctt | ctcgcggtcc | 62760 |
| taactgcaaa | gcccgctggc | cgccctagca | acgccacctt | ttccgcctgg | gacgcttacg | 62820 |
| tggctgcgaa | caattcttgc | tctcccctcc | aggtcattct | tgctccccta | cagcagcggg | 62880 |
| gcaggcagcg | gcatcgctct | ctggccgggt | accaccgcaa | gggtgctggg | gggctccagg | 62940 |
| agtggggtct | gtttcctccc | ctctggaggc | agggtttccg | tgagaccgac | cccagggaag | 63000 |
| aagctgcggg | aaaagtgcca | ggagggggct | gaacttacgt | agaggttgct | gaatcgggct | 63060 |
| tcgtagaagt | gcaggggatt | cgccagcttc | accacggctg | agctttcgtc | gtcgccttcc | 63120 |
| tgcaggagct | ggtcccccca | cgactccccg | tgtgggaaga | gctcgtctgg | gtgcagcgcc | 63180 |
| gcggcccgca | acattagcaa | cggcagcagc | agtagcactg | gtaacgacga | cagcaccggc | 63240 |
| cgcccggcca | cccggtcccc | ctccat | | | | 63266 |

Following is an ENDO180 genomic nucleotide sequence (SEQ ID NO: 3).

| >17:61200451-61268650 | | | | | | |
|---|---|---|---|---|---|---|
| 1 | catattgtgc | tgtgcttcaa | atgtgtctca | tgtctcctat | gcagcgtgtc | acagtgtcat |
| 61 | ttgacatcaa | tcatgttggc | agggcagcag | accccccccg | ccccaccgat | acatacattt |
| 121 | tttgttttgt | tttattttga | aatagataca | gccttcttgt | cattcttttt | tttttttaat |
| 181 | tttttttaaa | atttttttga | gacaagagtg | tcgctctatt | gctKaggttg | gagtggcact |
| 241 | atctcggctc | actgcaacct | ccgcctccag | ggttcaagtg | attctcctgc | ctcagcctcc |
| 301 | tgagtagctg | ggactacagg | cacccgccac | cacacccggc | taagttttgt | atttttagta |
| 361 | gagacggtgt | ctcaccatgt | tggccaggct | ggcctcgaac | tcttggcctc | aagtgatctg |
| 421 | cctgcctctg | cctcccaaaa | tgctgggatc | acaggcatga | gccaccatgc | ttggccgtca |
| 481 | ttcttaatag | acaccaggat | actgaaaaat | gctttgccac | tcagaaaaac | aagggtacaa |
| 541 | gcatgatact | atatggccac | tctcattcca | cttcagtgtg | tgtgtatggt | gggggcacaa |
| 601 | aagggatggg | tggagactgt | ggcaaaccgg | tgagcccaca | cccctcccag | gtgggcagct |
| 661 | gcaccagctc | ttgtggctgg | caccctgcag | gcatgctgcc | tcgactggaa | atctacattt |
| 721 | ttatgtgtaa | tctctctcac | acatcctgaa | gccaaacaca | acttgcctgt | gggctatcat |
| 781 | ctaaaattcg | gaggctagct | ggagtccgct | ctggaataag | tctgcctggc | catccttccc |
| 841 | agctcttccc | cttattaact | gtgttatcaa | gatgttaatc | tctcagttat | ttaatacctc |
| 901 | agtttcctca | tctgtcaagt | gcagatcaga | attttgccta | catctgaggg | ttgttataag |
| 961 | gattaatgaa | aagccttgga | acccactgtc | tggtgcctgg | acgcgcatgg | ttaatgcctg |
| 1021 | gcattctttg | tgtctctttt | tacaagcgca | gggtgaccac | aggtcccagt | ttgccggggg |
| 1081 | caggcctggt | tcatgcttgt | ggtgctggtg | taatgatcac | taacttgcca | tttcattgtc |
| 1141 | ggtattgacg | taaatgatat | gatttgccta | gagaagcaaa | tactggcaca | gtgctgttta |
| 1201 | ggactctgat | gaggtttaga | cctaatagaa | aggccatggt | ccaggttcct | tgaacatccc |
| 1261 | tggacaagtc | acttaacctt | tctgtgttca | gtttcccgtt | ctaaacgatt | ggcaagatga |
| 1321 | attctaagat | ccctccattc | acagtcattc | tgtcatttga | caaacatctt | tttttttttt |
| 1381 | tttttaaacg | cagtctcgct | ctgtcgccag | gctggagtgc | agtagtgcga | tctcggctca |
| 1441 | ctgcaacctc | cgcctcctgg | gttcaagcaa | ttcttctgcc | tcagcctccc | aagtagctgg |
| 1501 | gactacaggc | gaacgccacc | acgcccagca | aatttttgta | cttttagtag | agacagggtt |
| 1561 | tcaccatgtt | ggccagaatg | gtctctatct | cttgacctcg | tgatccaccc | acctcggcct |
| 1621 | cccagagtgc | tgggattaca | ggcgtgagcc | actgcgcccg | gccgacaaac | atctttcaca |
| 1681 | agctcagact | catgccaggc | aaaggtccag | ggcatggaga | agccaggggc | agagcctggg |
| 1741 | tcctcagtgg | gcacttggcc | agtgaaaacc | tccagtgcag | gttgttggga | atctctacct |
| 1801 | tttttgtcaa | gtgttacttg | aaagaccatt | accaagaaat | tgaattgaaa | gttaattttt |
| 1861 | ttctccttct | ttaYggagac | gagtttcact | ctgtccccca | ggctagagtg | cagtggtgcg |
| 1921 | atctgggctc | actgtaacct | cctcctcccg | ggttcaagca | attctcctgc | ctcagtctcc |
| 1981 | tcaggcatac | accaccacat | ctgccaccat | acccagctaa | tttttgtatt | tttattagag |
| 2041 | atggagtttc | accatgttgg | ccaggctggt | ctcaaactcc | tggccttaag | tgatccacct |
| 2101 | gcctcggcct | cccaaaatgc | tggaattaaa | ggcgtgagcc | accgcgccct | gcctgactgt |
| 2161 | taatatttta | aacctcagtt | tctcaggaac | taatcattct | ggttgaatac | atttYgctac |
| 2221 | acatttcttt | tgtttgtggg | ggggtatcta | agttgttttt | ttcttttaag | tcagaaaaca |
| 2281 | taaattttca | ctatggaaaa | ttgtaaatta | cagaaaggcc | caaagatttt | cttcatttct |
| 2341 | gctgactctt | caatgaacca | gaggggcacg | tgtaatattt | ccgctttcgg | ttactcccca |
| 2401 | ccccacacac | cttgtagaca | ctttgtcaaa | actacatttt | aaaaaaatgc | tttcccccaa |
| 2461 | attcgtatgg | atgtctccta | ataaagtcac | gcagtcactc | actggttcgt | cctcaatcaa |
| 2521 | ccaggcgtgg | agtcctaacc | tgtccatgat | tttaagaaag | ggcaggcgca | caacaccccc |
| 2581 | gcacggagaa | atgaacaggt | gtggctcggc | agaggccttc | cgcacctgcg | ttatggagtg |
| 2641 | gtggccgctg | tcggcgatgg | ggtccccgcg | agggcacaag | tgatcgcgtg | gccacggcga |
| 2701 | ggctctgcct | ccgcccccgc | gctccgggtc | ccattcccga | attctgccct | cagcggcctg |
| 2761 | gtcaatgagg | ttggatcgtt | ggcccagaaa | gccacttcct | gccgcgactg | gagccagccg |
| 2821 | tcgttgtgaa | aattcccccc | acggggaagg | aagtggctct | tgggctgttt | attttggctg |
| 2881 | cgggctagca | gacggcgcca | ggggcagcgg | ccacgcagac | cgggcatttg | tctagctggg |
| 2941 | cgggaggcgg | cggagcggga | agtgccacag | attgaggggc | gcacggcggt | ggagggaggg |
| 3001 | gcgcccagtg | gagagggggc | tcacggtgac | agaggaagtg | gcgccgcttg | ggggggtgca |
| 3061 | cgatggcagg | gggaggggtg | ctcggtggag | ggaggggcac | acggtggcgg | ggggaggggc |
| 3121 | gtgccgcaga | ttgagggcga | acggtggctt | ggggaggggc | gctccttgga | gttgggggcg |
| 3181 | cacgatggcg | ggggcagggg | tgccccttgg | gagcagaagc | agagaagact | cggaactcca |
| 3241 | agagcaggtg | gatctgggag | ccccagatcc | tgggcactgg | gttgggggag | aaaggggtca |
| 3301 | aggtgtgccc | gcccccggga | tgctcggact | ctggggatcc | aggcctggtg | acccccggtg |
| 3361 | aggaagagcc | agtgggagga | aagggccggg | cgctgcccgg | agcagaggct | ggaggcgaga |
| 3421 | gcgattgctg | cttgctgtag | cgcggccgct | ctccagttcc | tccggcaagg | ggggcctggg |
| 3481 | ggaggactgg | ccgaaaatac | tgtgatgctt | tggtctaaga | caaaagccag | caggggatga |
| 3541 | gatcaaacac | catgacactg | cgaaggggaa | ggccagcgcg | ggtgaggatt | tgccttccca |
| 3601 | gagaacccca | ggggcggggc | tagcttccac | tcagcccgca | ttacccccac | ccccgccccc |
| 3661 | acccccgggg | cggggtctgg | aggatcagca | aaaccgcggg | aaaggtgaca | gtcgccttcc |
| 3721 | ccaccaccgc | ctgcatttag | taggtggagg | gacagtccca | cctctccacc | cattccctgc |
| 3781 | cagagtgtga | tttaatgtct | tcctagaaca | caggatatca | cagtctgcag | cacagagatt |
| 3841 | tgaagtgggg | agaaaagcat | ctctggaaat | gactggtggg | ggaagcccgc | cactgctcta |
| 3901 | caaagggtgg | cctgtgacag | caacagcgca | acactgggac | tgtgtgtgta | ggttttaggc |
| 3961 | acaagcactt | taagctgtgg | gaggaaagca | gtttggcctg | gctgggatgg | aggggctgtt |
| 4021 | ggggaggagg | gaggagggtg | tgaaaagggg | aaggtggccg | gtagagggaa | acccaccatg |
| 4081 | gttcaggatg | tcggtgggga | tgttgcacag | ttgggcgggg | tattgggctt | cgatatcatg |
| 4141 | taacccccgt | cctccatgga | agcaatgccc | ccctcgtcca | gcccaggatc | taatgtgcat |
| 4201 | gcatgtacgt | ggacacagtc | tctagccagg | ggtggtgcag | ggtggggtgc | tgggtcccca |
| 4261 | gggttggcct | cttagcaggg | tggggctggc | agcttctgct | gactcaagga | catttccagg |
| 4321 | cctggcctgg | aggttaagca | gctcattgaa | aacagctgct | cctcttggct | ggcggccctt |
| 4381 | tcaggcggcc | tctggctctc | cctgggatga | cccctcgcct | ctgcgtggcc | tggggagggg |
| 4441 | cagtgaaggg | agagaggctc | agaggctgga | ggccaccctg | ccatgccctc | agggatggaa |
| 4501 | ctgagtcccc | tccagggctg | gccagctgtc | ctccagcccc | cagctttcca | ggagctaagc |
| 4561 | caggtggtcg | ctgggccctg | cgttcctgcc | tgcagccttt | gctcctgcgg | ttgggcagct |
| 4621 | tggaccgcct | tccttgtcct | tggaatgcct | tccttgtcct | tgtctctgct | tgtgcctttc |
| 4681 | agggttttat | gtgacatgcc | tctcattcaa | cacatatttg | ttgagagtcc | gctgcatgcc |
| 4741 | aggcaccatg | ctagaattaa | cttctcatct | gcctccttca | tctgtgatgt | cttgtggaga |
| 4801 | gcagccgtct | tatctgaggt | cagacctctg | gctgcctctg | tggatgggct | gagtccgggg |
| 4861 | aaactgcagc | aacagctgag | ggttttgctc | cagagacaca | aactgttgtc | aagcctttgc |
| 4921 | actcagcctc | gcgacttcac | caaagcattg | tgtgccttga | agcagacagg | acgggagagg |
| 4981 | aggcctcagt | gaccatcaag | gaggaaggag | atgggttact | caatgtaggg | caaattgctt |
| 5041 | taaaacccag | ttgcccatga | gcacaggcat | gtttcccagg | tgatgacctg | gatgtctgag |
| 5101 | aaacaagaag | tgtggactac | atcagaaata | ttcctatcaa | actagacacg | gtggctcaca |
| 5161 | cctagaaatc | ctgcactttg | gaaggccaag | gtaggaggat | caggaggttg | gggccaggag |
| 5221 | ttcaagatca | gcttgggcaa | caaagcaaga | ctccgtctct | acaaaaagta | aaaacaatta |
| 5281 | gctggggctg | cgtgcggtgg | ctcacacctg | taatcccagc | actttgggag | gccgaggcag |
| 5341 | gcggatcacg | aggtcaggag | atcgagacca | tcctggctaa | tgcggtgaaa | ccctgtctct |
| 5401 | actataaata | caaaaaaatt | agccgggcgt | ggtggcgggc | gcctgtagtc | ccagctactc |
| 5461 | gggaggctga | ggcaggagaa | tggcatgaac | cccaggggtg | gagcctgcag | tgagtggaga |
| 5521 | tcgctccact | gcactccagg | ctgggcaaca | gagcagactc | cgtctcaaac | acaaacactt |
| 5581 | tgggaggcca | aggtgggtag | atcacgaggt | caggagattg | agaccatcct | ggctaacatg |
| 5641 | gtgaaacccc | gtttctacta | aaaatacaaa | aaaattagcc | gggcatggtg | gcgggcgcct |
| 5701 | gtagtcccag | ctactcggga | ggctgaggca | ggagaatggt | gtgaaccctg | gaggaggagc |
| 5761 | ttgcagtgag | ccgagatcgc | gccactgcac | tccagcctgg | gcaacagagc | gagactccat |
| 5821 | ctcaaaacaa | acaaacaaac | aaacaacaat | tagctgggca | tggtggggtg | catgcctata |
| 5881 | gtcccagtta | ttctgggggc | ttaggtggga | ggatcacttg | agcccaggaa | ttcaaggctg |
| 5941 | cagtgagcta | tgatcatgcc | actgcactcc | agcctggaca | acagaacgaa | actctgtctc |
| 6001 | tttaaaaaaa | aaaaaaaaaa | gaaaaagaaa | agaaaaaaag | aaagaaagaa | atatttctat |
| 6061 | cacacttcat | ttatctggaa | agttcatttc | tatctatcac | cttcaccccc | aaccgtgcag |
| 6121 | acttaaactt | ttccatggat | tgcttcatga | gttcagcctc | tccagcttca | ttataaacgc |
| 6181 | atgagggcag | aggctgtatc | ttcttttgat | tcctaggctg | gaatgcagtg | gcgtgattat |
| 6241 | agctcactac | tgcctcagac | tcctcggctc | agttattcct | cctgcctcag | actcccgagt |
| 6301 | agctgggacc | acaggcacat | gccaccacac | ctggctaatt | tttttttgag | atggaacatc |
| 6361 | actctgttgc | caggttggag | tacagtggtg | caatctcagc | tcactgcaac | ctccgcctcc |
| 6421 | tgggttcaag | cgattctcct | acctcagcct | cctgagtagt | tgggattaca | ggtgtgcgcc |
| 6481 | accatgcccc | gctaattttt | gtacttttag | tagagatgga | gtttcaccat | gttggccagg |
| 6541 | ctggtctcaa | actcctgacc | tcgtgattca | cccgcctcgg | cctcccaaag | tgctgggatt |
| 6601 | acaggtgtga | gccaccacgc | ccagcctaca | cctggctaat | ttttaaattt | ttttgtagag |
| 6661 | acgggatctc | accatgttac | ctaggcttgt | cttgaactcc | tgggctcaaa | cgatcttccc |
| 6721 | accttagcct | ttcaaagtgc | agggattaca | ggcatgagcc | actgtgccca | gcatctggtt |
| 6781 | catttttaat | ttcccttaca | gtgcttcata | aatgattatc | atctgtatgg | gataagaaag |
| 6841 | cccccaaggg | agcagccaga | aggatgatta | ggagaatggg | ctgagggtcc | agcctccagg |
| 6901 | cacctcctct | gacatggcct | tggtgctgca | gccgacccaa | gaggctggtc | tgacaggtca |
| 6961 | gaggaggtcc | tagagcagta | acctcccagg | aaataaggag | aatgggacag | aaacccaatg |
| 7021 | cagacaagct | aattaaaaaa | ggaatgcagg | agcccagctg | gctgcagatg | atcctgagtg |
| 7081 | tttcacagaa | tggaggaaag | agcctcaggg | attcagggac | aggacatcca | gcatctttgg |
| 7141 | gactcacttc | ctctctttcc | atctctttgt | cccgtttcac | tctgcttctc | tttatatatt |
| 7201 | atcccctcat | cgtaggaagc | atggccattg | gcagccccag | cctagcaatc | ttaacaaaaa |
| 7261 | gggaaagctt | tatgtgttcc | aggggagttc | tctgatcagc | cctgtttgag | ttatgtgccc |
| 7321 | catcactgtg | gccaagggga | tagacactgt | catcagctca | agagtgagcc | ttgtgcccac |
| 7381 | ccacacatcc | acatagccag | aggtacacaa | tacaaggaga | acaagaaggg | gatgggaagt |
| 7441 | ttcctgggca | gataaaggca | gctcaatgag | gtcacagatt | gttatcgttg | ccatgtcttc |
| 7501 | attgtagagc | ccagcgccct | gccagaccat | atggtgataa | gggctgggat | catgtctata |
| 7561 | ttgttcacaa | taccctcagt | gcctRatgcc | ctgctggcat | ctatgaaatt | ctcaatacat |
| 7621 | cattgttgga | tgaatgaatg | aataaatgaa | tgaatcacac | ccagccagtg | ctcagtgcac |
| 7681 | agctccctgc | ctagcttcat | tcatttctcc | atttactcag | taagaacata | ttgagccaca |
| 7741 | gcttgatgcg | tagcactgtg | ctaggcatgg | gttgattgcc | tacaaaatgc | aagaagtggt |
| 7801 | ctttgtcctt | ggggaggaga | atcccaagat | ccttgtgagg | ggccctgcac | cttctgtggg |
| 7861 | gggcactttt | gctccctgca | ctgacacctg | atgctcccat | gcatatggat | gaggctgggg |
| 7921 | ggtgggtgag | gctggagggg | ccctctctgg | atgggggtgg | ggggcatcag | gcctctgcac |
| 7981 | tctgagctcc | ctctgcccgc | tttgtatgtg | cttgatgaag | cacgSgtaca | aagaacacag |
| 8041 | agccagctct | caggacgcct | tttctctttc | cccagcagtt | ccagcccagc | ctgagcgtgc |
| 8101 | actccccctc | cactaggcct | tggagaaaga | gcctcttttc | attgagggcc | caggcccacc |
| 8161 | ggccacttcg | ggtggaaaat | gctctcaaga | accccctgac | cccggcttgg | cagagtcccc |
| 8221 | ccggatccac | ctacaggaat | cccccctcag | cttctccttt | gctttgcaga | gctcctgcac |
| 8281 | gcctctcctc | tgctttttga | ttcctcagcc | ttttctcatt | tgctgccaaa | tgagaaaatg |
| 8341 | caagaggttg | cctttgtcct | cttttgctgc | caaatgagaa | aatgtgacct | gtgttcttca |
| 8401 | gtgcctcctc | cacattgcag | tcttcagatc | tctgcccagc | ggagtctctc | ggcccttggg |
| 8461 | aggcacagag | ctccttgtaa | cgcccccaga | cacctgggat | tttaaaggtt | tgtttgctct |
| 8521 | tatgcccggc | atttctcctc | taactggatt | acaaatcctg | cgaggggagg | gacggtgctg |
| 8581 | cgagtttcct | tgtggtccca | gcattgcctg | gaacatgcca | tgcacctagc | agaaagttgg |
| 8641 | gtggatttga | acccagagcc | taggatgtca | gagggaggag | gggcctYgaa | gctgtgcctc |
| 8701 | atgtgacaaa | ggaggaaact | gaggcctgaa | aggtacttct | gttggacatt | ctaatcccgt |
| 8761 | cccctttctg | gacttcagtt | tcctctgtca | agtgagggga | ggttggcctg | accctgggaa |
| 8821 | aagcccctcc | cgactaacct | ggacctcttt | cagacccttc | attcaccgtt | tgctacaact |
| 8881 | gctacagccc | acctggcatt | gatttagcac | acttgtggat | tttctggctg | gggaggagag |
| 8941 | gaagataacg | tcctcttggg | ctattgtggg | ctcacgtgaa | gttttgggct | tcgttttcca |
| 9001 | gtcgtatctc | tgagaaacat | ctacaaaaca | agcaataatt | tttttccttt | tccggcccca |
| 9061 | ggcccctttg | gctacctcct | gccaaggcca | ttttctgtta | ctactgcctg | gagtagagtt |
| 9121 | tctgagaacc | agatacctac | tccttaaagg | aagcttgggc | aggcatggat | caagtcctgg |
| 9181 | agctggtttc | tggtttctgg | tgggtggctg | aaaggtgacc | tagtctgttt | gggttgttat |
| 9241 | aacacaatac | cgttaacaga | tggcttgtaa | acaatagaaa | tgtatttctc | atggctttgg |
| 9301 | aggctgagaa | gtctaagatc | aaggttcctg | ccgattcaat | gtctggtgag | ggccacttcc |
| 9361 | tcgtggacag | ctgtcttctc | actgtaccct | cacatggtgg | aataggtgag | ggtctctctg |
| 9421 | gggtgtcttt | tattaggacg | ctcatcctat | ccatgaaggc | tcttccctaa | tgacctaagc |
| 9481 | acctcccaaa | ggtctcacat | cctaatgcca | tcaccatggg | ggtttaagat | ttcaacacag |
| 9541 | gccgggcgtg | gtggctcact | cttgtaatcc | tagtactctg | ggaggccaag | gtgggaggct |
| 9601 | cacctgaggt | caggagtttg | agaccagcct | ggccaacatg | gtgaaactgc | gtctgtacta |
| 9661 | aaaatacaaa | aattagctga | gcatggtgac | gcaggcctgt | agttccagct | acttgggagg |
| 9721 | ctgctgaggc | agaagaatcg | cttgaaccgg | ggaggcggag | gttgcagcag | tgagcagaga |
| 9781 | tcgcgccact | gcactccagc | tggggcaaca | gagcgagact | ctgtatcaaa | aaaaaaaaaa |
| 9841 | aagctttcag | cacatgaatt | tttggaggtt | gacacattct | gttcatggca | ggagggaagg |
| 9901 | agcaggttca | aggcagggga | cccagagatt | agctgtggca | gttgtcctag | gggtacttta |
| 9961 | ggaagctcac | tttgtcctgc | ccagagagga | accccatcca | agtgctgtta | gaggacagag |
| 10021 | gggcagggtc | cctgccccaa | cctacattcc | catggtggtg | ttttgagcat | cagtgacagg |
| 10081 | agctgtgggt | actatgtttt | aggaaaatta | atctggctgt | tggaacaagg | agcagaaaga |
| 10141 | taggctggtt | gtttgtgatt | attatctagg | catgaaattc | gttcattcat | gcatgtgttc |
| 10201 | atttgttcct | tcagtattta | agcatcactg | tgtgtcaggc | agaactgtag | gtgctggggt |
| 10261 | tacagtggtg | gagaagacag | aaaacaagag | tgtcagatag | cgagttctgg | ttctggaagt |
| 10321 | ggcagagtag | cttgtgtcag | tctaactctc | tggccaagaa | tgcttttaga | ctaagggaaa |
| 10381 | attataaaat | atggtgatta | tggtgatttg | aaggcaatga | acagcaatta | aaaccagaca |
| 10441 | gaggtagaga | ggaaaaaatc | cttgaaagta | aaaagtaaac | ctggcacatt | taaccagctt |
| 10501 | ttccctgaac | acactacctg | gtttgaggag | ctcatacaga | aagtggcagt | tttaccggtt |
| 10561 | agagcttggg | gccactagaa | aggctaacat | tgagaggaaa | tgttaggaaa | gcaaggagcc |
| 10621 | atagaaattc | cttctacaga | ttctccttaa | atccttggtg | actcctgaac | tgtgcatttg |
| 10681 | cagggtaggg | ttctaagcag | cccagcaaga | aacagcagct | gggaggtaga | ggtttcagtt |
| 10741 | gctatctggt | gctgaggaaa | cagagtttaa | aatttaagtt | ccaccaagtt | tgaggggctt |
| 10801 | agtaatcacc | taaggttttc | ttttgaaatc | ccagaagggc | ctcaccttat | gattaaatcc |
| 10861 | aaggttaaga | gccacatctg | aagactaaag | gcaaaactca | aacaacccca | ccctagcaaa |
| 10921 | gcttaaaacc | aaacctccac | aagagcaagg | taatccactg | gtaatttaac | tgcctgccat |
| 10981 | aacaaaatcc | agcattcttt | agaggaagat | ttttttaaaa | tccagattcc | ctttaacata |
| 11041 | gcatccctga | cgttcagtat | acaacaaaaa | agtccagact | ccttatgaca | ttatccacaa |
| 11101 | tgtccagcat | ataataaaaa | tttgcagata | gtaggcagca | ggagatgacc | cataatcaaa |
| 11161 | agaaaaaaca | gtcaatagat | gcagatgcac | agacaaccca | gatggtgaaa | aaaatcagat |
| 11221 | aataaatgcc | atgaagagca | ttataatcaa | gtgatatgat | agagagtaac | tgtggtcact |
| 11281 | ttacgattgg | gaggccagga | ggatctctct | aaagatgtta | cccttttttg | ttgtttgaaa |
| 11341 | tgtgaaatgc | caggtagagg | aaataatgca | taggcccaga | gataggaaac | agccaggctt |
| 11401 | gttcaaggaa | gaacaaggag | gtcaaagcga | cttgagcaag | gtggagtaac | aagatttgag |
| 11461 | atcaggccgt | tagacaggac | cagatcatgt | taaattgtgg | ctcatagtaa | gaggctgaat |
| 11521 | ctcatttgaa | gaatgatgaa | ttatctttga | agtaattcaa | gcaggaggaa | gacttacctg |
| 11581 | ccttctgttg | ttgttgtttt | tttccctttt | actatgagcg | aggctgtcat | ttcaaatatt |
| 11641 | taagaggcaa | ttgcattttc | ttcttttgct | gtctatgcat | atctcttctt | cattttctac |
| 11701 | tgcattgttg | atactttccc | taagtttcat | gtatctattt | ttttggtaga | agctcttctt |
| 11761 | tcttttcttt | tctttccttc | cttcctttct | ttcttttctt | tctttctttc | tctttctttt |
| 11821 | tctttctttc | tctttctttc | tttctccttc | cttccttcat | tccttccttc | cttccttcct |
| 11881 | tccttccttc | cttccttctt | tcttaagact | agtaaagtca | gaaaggagga | aagagtagaa |
| 11941 | caaagacttt | ggtctgtaac | tgactatgaa | caaccaattg | agataactca | ccatctttgg |
| 12001 | accagccaga | ggctctttta | atattgtgga | cttaactatt | tgcctctaat | aaggcacata |
| 12061 | tatttttgtc | atttatttga | attttgacct | tgctgatggc | atcatatgtt | ggcattaaaa |
| 12121 | aaatcttttt | atgggaaaaa | agttcttatg | ctattgaatt | catcagtgtt | gtctatgacc |
| 12181 | ttcaacattc | caataatatt | tttaaatttt | ttctaggact | gatttatatc | tttaaaagat |
| 12241 | tattctaggc | caggtgcggt | ggctcatgcc | tgtaatccta | gcactttggg | agtctgagga |
| 12301 | gggcagattc | cctgagctca | ggagtttgag | atcagactgg | gcaacatgga | gaaacactgt |
| 12361 | ctctactaaa | aatacaaaaa | aattagccag | gcatggtggc | atgtgcctgt | agtcccagct |
| 12421 | actcgggagg | ctgagacaga | agaatcgctt | gaacctggga | ggcagaagtt | gcagtgagcc |
| 12481 | aagattgcgc | cactgcactc | tagcctgggt | gacagagcgg | gactctgtct | caaaaaaaaa |
| 12541 | aaaaatagat | tattctgact | gtctttggag | aatggactat | aataggatga | tgagatcagc |
| 12601 | atSggggaag | agagagaact | tgagatttgg | acccaggaga | tgagggccac | atttcagctc |
| 12661 | cttcccttaa | caatcttacg | actcttgtca | tcaaattgct | tcaccaccct | cagccaattt |
| 12721 | ctttaccaat | aacatagcat | aatgctgatt | tatagtcttc | ttactgtgcc | agatcctgtt |
| 12781 | ccaaatgctt | tacatgtatt | atctcatttc | atcgttacca | gagtcttaag | atgaggaaac |
| 12841 | ggcatagaga | gatgaagcac | tttgcccaag | tcccatattc | agtggcagac | ctagaatcga |
| 12901 | agcactagct | gccgggcacg | gtggctcatg | cctgtaatcc | cagcactttg | ggagaccaag |
| 12961 | gtgggcggat | cacccgaggt | tgggagtttg | agaccaccct | gaccaacatg | gagaaatctc |
| 13021 | atctctacta | aaaatacaaa | aaaattagcc | aggtgtggtg | gtgcatgcct | gtaatcccag |
| 13081 | ctactcagga | ggctgaggca | gaattgcttg | aacccaggag | gcagaggttg | cagtgagccg |
| 13141 | agatcgagcc | attgcactcc | agcctgggca | acacgagcaa | tagtccgtct | caaaaaagaa |
| 13201 | acaaaaaaaa | gaaacaaaag | aaaataaaca | ctagcagtcg | gattcatcat | ttcaacacat |
| 13261 | gaaaagtatc | ctggggagct | agtggctagt | ggatgcaaaa | gttctttgga | gattggatgt |
| 13321 | actccatatg | tgtgttgtgt | acataagtgg | cagtcaggga | acaagaggaa | ggtgatggat |
| 13381 | gtcacagaga | tctgggtttg | cttactgggc | gtgggacact | gggagtaggt | cacttcatgt |
| 13441 | ctctaagact | ctgtctgtct | tttcttacct | tccttccctc | cctccctccc | tccctcattc |
| 13501 | cctccttcct | accatccatc | cctccctccc | tccttcctcc | ctagtttctt | cctttctttc |
| 13561 | tctttcctcc | ttcctaccct | cccttgctcc | ttcctttctt | tttcttcact | gaaacataac |
| 13621 | tcacatatag | aatttaccca | tttaaaacat | atatttaatt | gggatttagt | tttttcacag |
| 13681 | cattgtgcaa | ctatcatgac | aatctaattt | tagaacattt | tcgtcatatc | cccaaaagaa |
| 13741 | gcaccgtgcc | ctttagcagc | cactcccctt | tctcgtcacc | cacaggcaac | cattaatttg |
| 13801 | ctttctgtct | ctatgcattg | aagcttcatt | tcttactctg | taaagtgggt | gtgctgtggt |
| 13861 | ggggggcagg | ttggatttcc | cccaRtgact | tctacagagg | gtgcctgctc | agcgaggaga |
| 13921 | taggtggaaa | gaggaaaggt | tgaggaagcc | aaagaattaa | attgctgcgt | aaaaattaag |
| 13981 | gcgtgaaata | gttcaggggt | gggatgagtg | gtctgggaag | atcctagaag | aaggaaagga |
| 14041 | tggaaccagt | gcaaggggca | gggggaaggg | tgagacacaa | tgcctccgga | tcaaaggaga |
| 14101 | attttggcag | aaaaaccacg | ccatgtaata | gagattgaga | ttgaggttgc | aacgtggtgt |
| 14161 | gtggtgtaaa | tgcctccact | cctcctcata | aaatcttcag | aaaagtatac | aggtgatgtg |
| 14221 | ccgggtggag | ttgggtttgc | tttttttttt | tttttttttg | agacggagtc | tcgctctgtc |
| 14281 | gcccaggctg | gagtgcagtg | gcgggatctc | ggctcactgc | aagctccgcc | tcccgggttc |
| 14341 | acgccattct | cctgcctcag | cctcccaagt | agctgggact | acaggcgccc | gccactacgc |
| 14401 | ccggctaatt | ttttgtattt | ttagtagaga | cggggtttca | ccgttttagc | cgggatggtc |
| 14461 | tcgatctcct | gacctcgtga | tccgcccgcc | tcggcctccc | aaagtgctgg | gattacaggc |
| 14521 | gtgagccacc | gcgcccggcc | tgggtttgct | tttgagggtg | gtacagagga | ctgagtcctc |
| 14581 | cctcctggtg | agcaggggct | agaacagagg | tagtcctgca | ggcctctgcc | tcatctgggc |
| 14641 | acaagggctt | agcactgcca | ctcatttcca | cctcagaggg | ttggtagtga | gtaccgagtg |
| 14701 | atttaacgta | tgagtgacgg | ctcaccatcc | tgcactgggc | acatggtagg | tgcctgtcag |
| 14761 | ccacaccggt | catcaagggc | caaccaggag | agcccctgtg | atgccttcgt | gtcttgcctt |
| 14821 | cccttccaga | acccaacRtc | ttcctcatct | tcagccatgg | actgcagggc | tgcctggagg |
| 14881 | cccagggcgg | gcaggtcaga | gtcaccccgg | cttgcaatac | cagcctccct | gcccagcgct |
| 14941 | ggaagtgggt | ctcccgaaac | cggctattca | acctgggtac | catgcagtgc | ctgggcacag |
| 15001 | gctggccagg | caccaacacc | acggcctccc | tgggcatgta | tgagtgtgac | cgggaagcac |
| 15061 | tgaatcttcg | ctggcattgt | cgtacactgg | gtgaccagct | gtccttgctc | ctgggggccc |
| 15121 | gcaccagcaa | catatccaag | cctggcaccc | ttgagcgtgg | tgaccagacc | cgcagtggcc |
| 15181 | agtggcgcat | ctacggcagc | gaggaggacc | tatgtgctct | gccctaccac | ggtgaggggc |
| 15241 | cgcttgcagg | cgggagggtg | gggtccccKa | tgtccagggg | actggagcca | tgagggacag |
| 15301 | attcccagtg | acaaggcctt | tggcctctgt | ggacccttgg | cagtcacatg | tttaattata |
| 15361 | ataggaagca | ttttttaata | attaagaaaa | ttaacttcag | gccaggtgcg | gtggctcacg |
| 15421 | cctgtaatcc | cagcactttg | ggaggccaag | gtgggtggat | cacttgaggt | caggagttca |
| 15481 | agaccagcct | ggccaacatg | gaaaaacccc | atctctacta | aaaattcaaa | aattagccag |
| 15541 | gtgtggtgga | gcatgcccat | catagtccca | gctacttggg | aggctgagga | aggagaatcg |
| 15601 | cttgaaccca | ggaggcagag | gttgcactga | gctgagattg | cgccactgca | ctgtgcagcc |
| 15661 | tgggtgacag | agcaagactc | tgtccccccc | cccacaaaaa | aaaaaagaaa | agaaaagaaa |
| 15721 | aaagaaaaat | taacttcaaa | gcatttaatt | tacttttgta | aaaattgcaa | aatacatgct |
| 15781 | tgtaaagtct | tcaaatagta | tacaagtata | taacataaaa | ttaaatgaaa | attcccttcc |
| 15841 | cccaggccca | ctctccaggt | gaaaccccat | tgtagccccc | catgttgtct | taatgcctgt |
| 15901 | cttgcatgtg | gtgaatgggg | accagagtca | ccatgtgagg | ggctggcctc | ttgtggtgga |
| 15961 | cagccccgtc | ctgggggggc | ctttcctggg | agaccccctg | agttccgaga | tagaactttg |
| 16021 | tggggacaca | gagcccatgg | gctgggtagc | tgagcagaga | ttccaccggg | ctcagccctc |
| 16081 | tgtgggtgac | ttggaggccc | tcttgcccag | ctggagtttg | gatgggatta | ggggtactag |
| 16141 | ggagcccctt | cccagcccta | tgaggtgtgc | agcaagacct | ctctctggtt | cctgggctga |
| 16201 | ctcaacagca | gaagaagcct | taaagccact | atggggtgcc | attgcctctc | ccacctgctg |
| 16261 | atccctgtga | acacccagca | ctctgggttt | taggggattt | ctcctgaggg | tcgaggggct |
| 16321 | tggcagcctc | tggtgtccag | atgccaaggg | cctggcccct | gtccaccccc | tgcagaggtc |
| 16381 | tacaccatcc | agggaaactc | ccacggaaag | ccgtgcacca | tccccttcaa | atatgacaac |
| 16441 | cagtggttcc | acggctgcac | cagcacgggc | cgcgaggatg | gtcacctgtg | gtgtgccacc |
| 16501 | acccaggact | acggcaaaga | cgagcgctgg | ggcttctgcc | ccatcaagag | tgagagctgt |
| 16561 | tggagccgtg | ggggcggggg | cagtgttcct | ggagggaggc | tggtgctgag | gggccccggg |
| 16621 | gcccagggtg | agatactgcc | ccctccccta | cctagtgtag | ccttttggtg | ggggagggtc |
| 16681 | tgcactcccg | agggaccctg | gagggggcct | gaaggagagg | gctgtcRtgg | tggcaggtaa |
| 16741 | cgactgcgag | accttctggg | acaaggacca | gctgactgac | agctgctacc | agtttaactt |
| 16801 | ccagtccacg | ctgtcgtgga | gggaggcctg | ggccagctgc | gagcagcagg | gtgcggatct |
| 16861 | gctgagcatc | acggagatcc | acgagcagac | ctacatcaac | ggtgagccgg | ggcctgatgc |
| 16921 | ctgctcgtgc | ctctggaggg | cccgggccct | ttccgcttgt | gggttgggga | gagggcgatg |
| 16981 | gggagcgggg | gaggctgggg | ctggggatcc | cctgttcagt | gtccctcctt | gctgtcaggc |
| 17041 | ctcctcactg | ggtacagctc | caccctgtgg | atcggcttga | atgacttgga | cacgagcgga |
| 17101 | ggctggcagt | ggtcggacaa | ctcgcccctc | aagtacctca | actgggagag | tggtgaggca |
| 17161 | caaggttggg | ggcgcagggc | agcatagggg | ccccgcgggc | tcttggcctc | ccatggactc |
| 17221 | ctctcctcat | gtcctcctgc | agaggggcag | tgtgggtagg | ggaagcaccg | acctccaccc |
| 17281 | ccctccccag | actgcgcccc | cctagcccat | gtagggcggc | gctgccccag | gccgaaggtc |
| 17341 | ttggtgcata | tggactgggt | gccagcctcc | actcacctct | tcagccaccc | ctgtgggcag |
| 17401 | cgggcaccag | cgcacccagc | agcctggacg | gggaggccgg | ggcggggctg | gtactggtta |
| 17461 | ctgggtagaa | ggttcaaggc | gacgtgcacc | caggttagaa | agcgcggagg | accccgtggt |
| 17521 | ctggggcgga | gctggagctg | agcaccaggc | ttcccgaact | ggctcaggct | tccgagggag |
| 17581 | cagagggagg | taggaggttc | tgagcagggc | cccgggagcc | acggtgtgag | cttctctctc |
| 17641 | cgggggtgct | ggcgccctgc | cctccccaca | gaccagccgg | acaaccccag | tgaggagaac |
| 17701 | tgtggagtga | tccgcactga | gtcctcgggc | ggctggcaga | accgtgactg | cagcatcgcg |
| 17761 | ctgccctatg | tgtgcaagaa | gaagcccaac | gccacggccg | agcccacccc | tccaggtgag |
| 17821 | ccagggactg | tgccgcaggg | tggggagggg | ctcccagggc | cagggacaca | gccacagagc |
| 17881 | cgtggcaggc | ccagcctctc | ctccggttac | caccacagca | caaggggact | ctggatcctc |
| 17941 | tgactcttat | ttcattgttc | agttaaagtc | tgagcaaatt | ggaatatgtc | attaattcat |
| 18001 | ttggggaaca | tgtgctgaga | tctgtgttaa | atgctggcaa | tacccactgt | tacctcgtag |
| 18061 | agaccacagt | ctgctggagg | tgacagacat | ctcaatgcag | ttagactatc | ccaggtcatg |
| 18121 | agtgattata | gaaatatgga | tcagtgttag | aggagcatag | agaagaaact | gattcagctt |
| 18181 | ctgagagtta | aggaggggtt | tatggaaata | acatttgatt | tgggcctttc | aaattgagtc |
| 18241 | taagtttaaa | agatataaag | aggtgtgggg | gaagggcatt | cctggcagag | ggaagagcat |
| 18301 | atgcaaaggt | gtggaggcct | agaactacac | tgcagcttta | aggggtggtg | agtgatggcc |
| 18361 | gggcaccgtg | gctcacacct | ataatccYag | cactttggga | ggccaaggca | ggaggattgc |
| 18421 | ttgagctcag | tagttcaaga | ccagcctggg | caacatagcg | agaccctgtc | tctgcaaaat |
| 18481 | atttttaaaa | aattagctga | gtgtggtgat | gcacgcctgt | ctactaggga | ggtcgaggtg |
| 18541 | ggaggattgc | ttgagcctgc | aaggttgagg | ctgcagtgag | cctcgcttgc | accactgcac |
| 18601 | tccaacctgg | gcaacagagc | gagacgagac | cctgcctcaa | aaaataaata | aataaataaa |
| 18661 | taaataaatt | taaaaaaaaa | aagaaaaaag | aaagaaaaag | aaaaaaatgg | atggtgcctg |
| 18721 | ttgcagtttg | ggtggagtat | agattctaac | cacttcaagt | atggtcatcc | aaccagcagc |
| 18781 | acagcatccc | cRgagggctt | gttagaaatg | cacagtcagg | cccgccccag | agtaattgaa |
| 18841 | tcagaacctg | catttaacaa | gatcctcaga | tgattcatgt | gcagacacag | tttgggaagc |
| 18901 | cctggcttaa | agggcatggt | gaagaagagg | caggttgcag | ccagtctctg | cagggtcttg |
| 18961 | gctgctgggc | caagtttggg | tggggccagg | taggctgtgg | ggagcctttg | aagatgttta |
| 19021 | agccagggag | tgtctggttc | tcgtttgtga | tcatagagac | agctttaggg | gcctcgtgga |
| 19081 | ggagttgggg | caatggacag | ggtctgcttt | agggattctg | ccccttactg | ctgtgtggcc |
| 19141 | ctgggcaagc | agcttagtct | ctggggtgca | gagtcctcat | tgcaaatgga | gaatRaagcc |
| 19201 | accccccttg | caaggttgtt | gtgaggaagg | ctggagatct | ggcctggcac | gaaggagttt |
| 19261 | ttctgtagtt | ggctgtggat | gtttactgtg | gtgtaggttg | gaggggctgt | gcagtttcct |
| 19321 | gcgaagctgt | ggggtggtga | ggagactttt | acaaagaaaa | tatggcaaca | cacacacaca |
| 19381 | cacacacaca | cacacacaca | cacacacaac | actaactaca | gacagtaaag | agtttacaga |
| 19441 | gcaaaacttc | tgctcctgac | catggaagtc | ttgccaatga | tcgactcagg | actggttttt |
| 19501 | ttgtttgttt | gtttgagaca | gggtctcact | ctgtcaccca | ggctggagtg | tagtggtgcg |
| 19561 | atcttggctc | accacaacct | ctgcctccta | ggctcaagag | attctcgtgc | ctcagcctcc |
| 19621 | tgagtagctg | ggattacagg | cgggcatcac | tacttcccgg | ctaacttttg | tatttttagt |
| 19681 | agagacgggg | tttcaccatg | ttggccaggc | tggtcttgaa | ctcctgacct | caggtgatct |
| 19741 | gcccacctcg | gcctcccaaa | gtgctgggat | tacaggcgtg | agccaccgca | ccccgccttt |
| 19801 | ttatttttat | ttttatttat | ttattttttg | aaacagagtc | tcgctctgtc | gcccaggctg |
| 19861 | gagtgcagtg | gcacaatctc | cactcactgc | aacctccgcc | tcccaggttc | aagcgattct |
| 19921 | catcYccagg | ttcaagcgat | tctcatgcYc | cagcctcccg | agtagctggg | attataggca |
| 19981 | tgagtgtgcc | accacgcccg | gctaattttt | ttgtattttt | agagatgggg | tttcaccatg |
| 20041 | ttgcccaggc | tggtctcgaa | ctcctgtctt | caagtgattc | acccgcctcg | gcctcccaaa |
| 20101 | gtgctggaat | tacaggcgtg | agccacggct | ctggcctgat | tcaggactgc | tgaccactgc |
| 20161 | tttatctccc | attccgtaag | gggccaggag | cagaagcttg | aaacgtcagc | aacccgatct |
| 20221 | agggcacctc | cacctctagc | tggagggagc | cttgggccct | agtccagtcc | tcccacaagc |
| 20281 | acctttctga | aggaaggggc | ccgctgggag | gcccgggtgt | gggcatgcct | agcctatctg |
| 20341 | tggcccctta | ccaagtcctc | tgtcctctgg | gtcttcccgg | acctgcagac | agatgtaagg |
| 20401 | cctcggattt | ccgcccctct | gcccagccca | tatctctctg | cacgaggcat | attctaggca |
| 20461 | ggttctgctt | tcccggtctc | atcacttaag | tcctgtcatt | tcgaaggcag | gcgccccgtc |
| 20521 | gtgtccccaa | agcatccaca | tttcccaagc | tcccttccgg | tccttgaggc | ccctcgggtc |
| 20581 | tcagcagcca | ctaaaatccg | cttgtctggg | gcggcagcct | caggtcttag | ctcattggct |
| 20641 | aacaagagat | gattgacagg | tcctgagcca | gagagcgagc | ccaccgggct | ctcagtccgc |
| 20701 | ccagctttgc | cgggtctcca | ggagcaggtg | tggctggacc | actggaatcc | cagtctgagg |
| 20761 | ccgtgttcta | gtgggcgcgc | agtttcctgc | ccgactaggc | agaatcctgc | ccgagcatga |
| 20821 | cactgtgggt | ggtttggtga | agtcctgttt | tctcttgcca | agaccaagta | agggcttgtt |
| 20881 | tgcctcagct | gccctcggaY | cccaacccct | agaaggtgaa | cctgaaaggc | atggaaatcg |
| 20941 | gggcaaggga | gggcctggga | cgggtggatt | tctcagccct | ggggttcagg | gttaggagtt |
| 21001 | aggagcgggg | aaggagtccc | agctggggga | tggggccgat | ctgtagtgac | tttgagcacc |
| 21061 | tcctaatgtt | ctcggttgcc | cgtccaccaa | gacctcagat | tcctggtggg | cttgcctggg |
| 21121 | ccccattagt | ggctcaaggt | cccgtttccc | agttttctcc | ctcctgggct | tcccagacct |
| 21181 | gcacagagtg | ggtgctcaat | aaatgttatt | tgagagaata | acagtgattc | catttactag |
| 21241 | gcagggggcc | ataagccaag | cttgaatctc | atagcaggaa | accccattta | cagagggaga |
| 21301 | aactgaggtt | catggggctg | gtttgtcctt | tctacccatc | gtttattgac | tgattgattg |
| 21361 | atgcattgat | tgagataagg | tcttgctgtg | tcgcccaggc | tggagtttag | tggtgcaatc |
| 21421 | atggctcact | gcagccttga | cctccccggg | tcaagtgatc | ctcccacctc | agcctcctga |
| 21481 | gtagcgggac | cacaggcgca | catcaccatg | cctggctaat | tttttatttt | ttatagagat |
| 21541 | ggggtctccc | ctttgttgcc | cagtctgttc | ttgaactact | gggctcaagc | aatcctccag |
| 21601 | ccttagcctt | ccaaagtgct | gggattacag | gcctgagcca | ccatgcctgg | cccacccatc |
| 21661 | gtattctgag | agccacagcc | agaggaagtg | ttctgtcctg | ggtggaatct | gccaccacga |
| 21721 | cttgggtctc | tgtcccgggg | ctccagcagc | cctgtaaagt | tttgtgtttt | tttagcattt |
| 21781 | ccaagacctg | tggtggggac | cgggattgat | ctgggagagt | ttggagagga | ggtgactggg |
| 21841 | aggcctYgcc | tgtgttgacg | tcaacccagt | gggttggttc | ccagactggg | cggctcagtc |
| 21901 | cctgagcagc | agcctcatgg | gtctctgcag | acaggtgggc | caatgtgaag | gtggagtgcg |
| 21961 | agccgagctg | gcagcccttc | cagggccact | gctaccgcct | gcaggccgag | aagcgcagct |
| 22021 | ggcaggagtc | caagaaggca | tgtctacggg | gcggtggcga | cctggtcagc | atccacagca |
| 22081 | tggcggagct | ggaattcatc | accaagcaga | tcaagcaagg | tgaggagctg | cccgcccacg |
| 22141 | tgtctgggtg | gagggcaggg | cctcccactg | ccccacccat | cctgtccagg | agcctcagag |
| 22201 | aatgttcctt | cctccctact | ggtggctgag | gctgacctct | caatgttttc | tctcccctcc |
| 22261 | tctcctgctg | cacccccaga | ggtggaggag | ctgtggatcg | gcctcaacga | tttgaaactg |
| 22321 | cagatgaatt | ttgagtggtc | tgacgggagc | cttgtgagct | tcacccactg | gcaccccttt |
| 22381 | gagcccaaca | acttccggga | cagtctggag | gactgtgtca | ccatctgggg | cccggtgaga |
| 22441 | tctccctctc | cctRtcacag | ggccacaaaa | tacaccccca | acctccaggt | gccaccccag |
| 22501 | ctgaaggaca | tcctagttac | tatcagaacc | tgcctggaac | ctcttacctc | tcagcagtcc |
| 22561 | ccctcctccc | caccaatgcc | ttcccttcca | tgtgagagac | tgccggggct | tgaatcctac |
| 22621 | ctccacctcc | acctccaagt | agccaagtca | ccttgggcaa | gttactagag | ggtcactggg |
| 22681 | gagatcagag | gagttaattc | tcgtgaaacc | ttaggcacag | tgcctggcgc | attcttggtc |
| 22741 | tcctcccagc | tgcccttccc | tgccagaagg | tggcatcaga | acccacatta | tgtgcagata |
| 22801 | ctttgcaagc | agggctgcac | tctgccagcc | ctcgccccca | gtgctctacc | cagtccaggg |
| 22861 | tcctgtcatc | tcaagagatt | gttgggtttt | atgacaattt | gccatggttt | gggcttagat |
| 22921 | gtggtgtaag | tgaaggaatt | gatggagggc | ttcaagcaga | gatgaaagac | ccttaaaaaa |
| 22981 | gggacaaagc | cagctgagtg | tggtggctca | tgcctataat | cccagcacat | tgggaggctg |
| 23041 | aggcaggcgg | atcacttgag | gtgaggagtt | cgagaccagc | ctggccaaca | tggtgaaacc |
| 23101 | ccatctctac | taaaaataca | aaaattagcc | aggcgtggtg | gcagatgcct | gtaatcccag |
| 23161 | ctactccaga | ggctgaggca | agagaatcgc | ttgaacctgg | aaggcagagg | ctgcagtgag |
| 23221 | ctgagattgc | accactgcac | tccagcctgg | gtgacagagc | aagaccctgt | ctcaaaaaaa |
| 23281 | aaaaaataaa | ataaaaagga | gaaagccgta | gggacacaga | ccacccttcc | tagggacaga |
| 23341 | caggatgttc | tgctgtcaca | tactgcaaac | agtgctgtgg | actgtagtgg | cagtcatttg |
| 23401 | ccttagggga | tgctatttaa | gaattttaaa | acaattatgt | attgagtatc | taccatgtgc |
| 23461 | cagtcatttt | tctgggtgct | aggatacaca | aacacgcctg | ccctcagaat | ggagcttaga |
| 23521 | gacagacata | ctgctggctg | ggggcctgtg | gatactgggg | agggcgaaga | cagaagcagg |
| 23581 | ggccgaggac | taaattcctc | ctcagcttca | tcagctgaga | ttctggtctg | ggcacactgg |
| 23641 | cctctgcaca | ctctggccct | gcaggcacta | gctagacgcc | ttgggaatcc | aagctgtcta |
| 23701 | tcctgccatc | agttactcta | acaatcttag | ccagtttgtt | tttgatattt | ataaaacgag |
| 23761 | ttatcaataa | tgtaaaagga | cgcctttcct | tttttttttt | ttttttttga | gatggagttt |
| 23821 | caccctgtcg | cccaggctgg | agtgcaatgg | cacgatcttg | gctcactgga | acctctgcct |
| 23881 | cccaggttca | agcgattctt | ctgcctgagc | ctcccaaata | gctgggatta | caggcatgca |
| 23941 | ccaccacaac | cggctaactt | tttgtatttt | cagtagagat | ggggtttcac | catgttggcc |
| 24001 | aggctggtct | tgaactcttg | acctcaagtg | atccgcccac | ctcggcctcc | caaaatgctg |
| 24061 | ggattacagg | catgagccac | cgcgccgggc | caggatgcct | ttcccattta | gacaatgctt |
| 24121 | gttaagtttc | aaggcgtttg | cacatgaact | gcttgtgctc | tcatccaccc | tgtgaggctt |
| 24181 | atgggttcat | ccYgccttcc | ccattgcaca | gctgggacac | agacatgtca | gaatcacaca |
| 24241 | gtaagtcaga | gacagggcca | gctctaggaa | ccagattcca | agaaggattt | gggagattta |
| 24301 | tggggaggtg | ggggttgaga | ttcttcctca | ccctgtgtcc | gtaggaaggc | cgctggaacg |
| 24361 | acagtccctg | taaccagtcc | ttgccatcca | tctgcaagaa | ggcaggccag | ctgagccagg |
| 24421 | gggccgccga | ggaggaccat | ggctgccgga | aggtgagggt | gtttctggag | ctgccctgag |
| 24481 | tggggccacc | tgtcagaggg | gctaccaggg | gagggagagg | tggatgaact | cctgctgcct |
| 24541 | cgcatggcat | cgccctctcg | tcggcacccc | cttccctgag | ctctccaaac | tgctccttta |
| 24601 | ttgaaggagt | gatactggat | gggatgggac | gggtgggcgc | tgttgggagg | aagcagagct |
| 24661 | ccccaccccc | agcccagtcc | tgagtccctc | cccatggggg | aagccacttc | ctcgagacag |
| 24721 | cagagaacag | ggaggtgggg | gctggtctac | ccccgagtgc | cccctcagag | tccagcctag |
| 24781 | tcatgtcaag | cagtggctgc | cgaagtgaag | tgtggcccct | gtgcaccgcc | agtcctgact |
| 24841 | tccccctcct | ctgccgtctg | acacagggcg | tgtgctggtc | tgtctcccct | ggaggagtga |
| 24901 | ggccactcag | ggtggggctg | caccttcccc | ctccttgtgt | tcccagcccc | agtgggcttc |
| 24961 | tagaggatgt | ttagggaggt | tgaggggggg | ggtgtcaagg | aatgaaagct | gtgtaagtga |
| 25021 | gtggggggtg | agtgactgag | ggtgtgaggg | ggtgacagac | cctctcctcc | tccagggagt |
| 25081 | ggagcagggg | tgcaaatgct | gtgatgggtg | cggggagctt | gcttctaaaa | gacactgctt |
| 25141 | ggcccttggc | cccccccagc | agccccttgc | agccaggctg | gctcctcact | ctgttttcct |
| 25201 | ttggattaag | cccctaccac | acccacctgg | accccaaagc | ctcccccaga | aaatgagcag |
| 25261 | gaaacactaa | caggcatggc | cccatgggcg | aggaagcaga | ggggagagca | gagcccagct |
| 25321 | gcggagtctg | tggactggca | aggacagtgc | ctggcccagg | ggctgtgtca | tggctgcgaa |
| 25381 | gaggtgaagg | gaggagtaga | gaggaattac | caacttctct | ggcaccaagg | ccagcctgga |
| 25441 | atttgtgtgg | ggagtgttga | gggtccatgg | ttaacagccc | aggatcacca | ggggtcttgg |
| 25501 | cacagcccct | gccagcagat | gaggccccca | ccacacatac | cacatctgct | tctcttctgc |
| 25561 | cttggctcct | gccagcctcg | cctgccaggg | tctcgtctct | gagcccctcc | tgccatcccc |
| 25621 | gggccaggag | catcctgcgc | cgactccagc | tttgccaggc | cctcgaggac | tgcccatccc |
| 25681 | ctctccatcc | tggcagctct | ccccagcctt | tagctgtggt | ttcccagcca | acagtaattt |
| 25741 | ccccactgtg | tctcttggtt | taaatgccca | agagaggagc | ctgaagggcc | attcatcttc |
| 25801 | ccgagccggg | tcactggccg | gtcgctggtg | gcctgccaat | cagccacgct | ggggctgggc |
| 25861 | ctccacccca | gcttccacag | ctgtgggtga | tggtggaggg | ggaatgtgga | gaaacgaggt |
| 25921 | gcagaacaca | gcccagtact | caaaccagtc | ccctccgtcc | tgagcacgtt | cagtgatgtc |
| 25981 | cctgatggca | tctcccacca | caggatttat | gggtgagggt | ggagagtcat | cttccctaca |
| 26041 | gacacccctc | ttctgtccac | tcttgagcca | gggttggacg | tggcacagcc | catcctgcta |
| 26101 | ctggctggga | gaagaccaag | tgacctacag | tgaggcccgg | cgcctgtgca | ctgaccatgg |
| 26161 | ctctcagctg | gtcaccatca | ccaacaggta | cagcaggggc | gggtgccctg | actagccctt |
| 26221 | gcccatgtct | gggcctattg | tggtcccttc | agcaaacagg | gtagcatctg | ccatcatgcc |
| 26281 | cagagggacc | tggagtcctg | tgaacctcag | tgggctccag | ggcattttaa | aatcttgtca |
| 26341 | gaagaatgac | tctcacttgt | taagtgaccc | cagtgtaggg | cagggcccgg | gcaggcacag |
| 26401 | gtgtgggctg | gatacttgag | aaagagaggc | cttggttgtg | tctgattttc | tctactttct |
| 26461 | tctcagcaaa | gcaaggggtg | cagagaatag | aaggggcagc | tgggggttcc | ataaatcagc |
| 26521 | tgcagggctc | ccaggcgtcc | ctgctttctt | gagcaagtta | ttggtcgggt | gcagcacccg |
| 26581 | agctcccacg | gtagggcgtc | tggcctgtga | ctgggggatt | gggaagcagg | gagatccctg |
| 26641 | ccctgaccag | cctgtctcct | gaaggttcga | gcaggccttc | gtcagcagcc | tcatctacaa |
| 26701 | ctgggagggc | gagtacttct | ggacggccct | gcaggacctc | aacagcaccg | gctccttctt |
| 26761 | ctggctcagt | ggggatgaag | tcatgtacac | ccactggaac | cgggaccagc | ccggtgagcc |
| 26821 | ccttMtttga | cttgccttgg | tgaagcgagg | agggaggcca | gggtggactg | gccctgccag |
| 26881 | caccgagccc | cagggcttcc | ccataaacag | atcattggtg | cactgtggtg | tagtgtctat |
| 26941 | gagcacaagc | tctgaaacca | aactgcctgg | ctccgatcat | gactctgcca | tcttttagct |
| 27001 | gtgcatcctc | tgagcctcag | ttttctcatc | tgtccagtgg | gaataatgat | cctttcctca |
| 27061 | tagggatgac | atgagtctta | aatatgtgta | aagtgctaat | atgtgtaaag | agttaatgtg |
| 27121 | tgtaaagtgt | ctagattact | tctctatctg | ggctcacaaa | aaagaaacaa | ataatctctt |
| 27181 | ctctatctgg | actcatttaa | aaaatgtgtt | cagaatactg | agcgcaatac | aagtgcttgt |
| 27241 | tattactatt | attaccattt | cacattcatt | atctttctaa | ccccttcaat | aacctgatga |
| 27301 | ggtagatgat | gaagcaggct | cggtgaggtt | aagcagcttg | cctgagccct | cacagtgggt |
| 27361 | gaacccagag | ccagggctct | gcctccattc | ccttcctccc | tcctggctcc | aaccccagag |
| 27421 | agcatgtctc | tgaagaggtg | gccaagctgg | ttcccagtgt | gagggccgtg | ctagtgccct |
| 27481 | gccgggatgt | gagggaggac | cagactatga | atccttctca | gagcctgggt | ctcccttccc |
| 27541 | tctccttcag | ggtacagccg | tgggggctgc | gtggcgctgg | ccactggcag | cgccatgggg |
| 27601 | ctgtgggagg | tgaagaactg | tacctcgttc | cgggcccgct | acatctgccg | gcagagcctg |
| 27661 | ggcactccag | tgacgccgga | gctgccgggg | ccagatccca | cgcccagcct | cactggctcc |
| 27721 | tgtccccagg | gctgggcctc | ggacaccaaa | ctccggtatt | gctataaggt | agggcagcct |
| 27781 | gttggccggg | tagggggcag | ggggaggaca | ggagcaaaga | gacagtttgg | atggaggtcc |
| 27841 | cagtgggacc | agccacaatc | cagagacaca | ccaggctgca | gacgggtgat | ttctggtgag |
| 27901 | acttgtccat | ggaatctgga | ggcctggtgc | tcagtaatca | tttgaggaat | tgagggaacc |
| 27961 | acagatcatt | gatttcagag | gttcccaaat | gttggggcca | gcctagctgt | tcttcagaat |
| 28021 | tgcttgggga | acctgataaa | aataaaatct | tggccgggcg | cagtggctca | cacctgtaat |
| 28081 | cccagcactt | tgggaggtca | aggcgggtgg | atcacgaggt | caggagatcg | agaccatcct |
| 28141 | ggctaacatg | gtgaaacccc | atctctacta | aaatacaaaa | aattagccag | gtgtggtggc |
| 28201 | aggcgcctgt | tgtcccagct | actcgggagg | ctgaggcagg | agaatggcat | gaacccagga |
| 28261 | ggcggagctt | gcagtgagcc | gagatcgtgc | cactgcacgc | cagcctgggt | gacagagcga |
| 28321 | gactccgtct | caaaaataaa | taaataaata | aataaagata | aaaatctagg | ctgtcacaat |
| 28381 | ggagatttag | attcattagg | tctggattgg | gcctggtaat | ctgtattttt | aatcacgcta |
| 28441 | ggtttgggag | ccactggcag | agtccgaggt | cttttattat | gcacagaaac | tgaagtctag |
| 28501 | ggagaagaag | ggacttaccc | acgttaagct | gtggctctgc | tgggattaga | gcccagatct |
| 28561 | ccctgctggt | caggggatgg | ggctcagggg | ccctctccag | aaggtgactg | tgccagctcc |
| 28621 | cctccccaga | cctctgcaga | tgaacaggat | tgtccttagc | tagcctKagc | cccctgtcca |
| 28681 | gtaaggctcc | aggcccctcc | tctgccatgg | tgggaaaggc | agtaggtgcc | ccctgagggg |
| 28741 | tgggccagtg | gggacagctt | agacagctgg | actctgcccc | tgcaggtgtt | cagctcagag |
| 28801 | cggctgcagg | acaagaagag | ctgggtccag | gcccaggggg | cctgccagga | gctgggggcc |
| 28861 | cagctgctga | gcctggccag | ctacgaggag | gagcactttg | tggccaacat | gctcaacaag |
| 28921 | atcttcgggt | actgagccgg | gctgaggcgt | gttaggaggg | cacccgcaca | cgtgtaggag |
| 28981 | caggcatggc | cgacagaccc | ttggttttgt | tgggcgagca | gggggatggc | agaatgtggg |
| 29041 | tgaccccagg | gaggcctgca | tctgctgctg | gtgccaggac | catcttgtcc | tcactcaagt |
| 29101 | agccatggtg | ccctggcatc | ttgggagaac | aggctggtgt | gatccatgca | ctcataggag |
| 29161 | actgtgtttt | gctcaccatc | atccccccag | caatgctcca | acagacccac | aaacttacat |
| 29221 | atgtaccccc | caccccattg | caaaatctcc | cacatgctgg | tcctggcaag | tgtttcccct |
| 29281 | caccttacac | catcctacaa | tgacccacac | tcctcaatcc | acagtttttc | ctgagagata |
| 29341 | ggaggagaaa | ggccgcagct | atgaggattt | gcccatgggc | ctccctctga | gacaccacct |
| 29401 | ctgtcctggg | ctctgagcat | tagcacgtgg | aatcccctag | cactgggaaa | atcgagatgg |
| 29461 | atctgggctt | cctgatagct | taattgaagg | gatctctcct | ctcagcaaac | attgaaatta |
| 29521 | cattatttgt | ctcataatta | caatagctca | tggtcatcac | ttctcaccac | gtaccaggca |
| 29581 | ctgtgctaaa | catatagtcc | tcactcaagc | ctcccgacaa | ccctatggag | cagacgttaa |
| 29641 | cccctgaccc | ttgtgggttg | atagagaggt | tatttgccca | agttcatggg | tgaacggata |
| 29701 | cttgaatcca | ggtttattta | actccagagc | cgtagatccc | tgccacctcc | tcatctgaac |
| 29761 | ttcctaaact | ccgtaaagta | aaagcctgca | gaatcttgct | ggacagggca | cccccaccca |
| 29821 | aaaggaaccc | aaacaaaggg | gaggggccac | tgccccttga | gtggatggct | tggtccagct |
| 29881 | ctccccagct | ctgtcttgtg | gggagaactt | taccgaagtg | acctcaacgc | tgtatcgatg |
| 29941 | ggtcaatgtg | aagcccagat | ggagaggccc | caggccctgg | gggctgcccc | ggtcacagct |
| 30001 | gcaagggaca | gggggcacgt | ttgggttcct | gcccctctca | cttcccatct | cttccgtccc |
| 30061 | cactcctggg | ttgtgctgaa | gtgaatcaga | acccgagatc | cacgagcagc | actggttctg |
| 30121 | gatcggcctg | aaccgtcggg | atcccagagg | gggtcagagt | tggcgctgga | gcgacggcgt |
| 30181 | aggggtgagg | gggcctgggg | tacttgggac | tgcgaggccg | ggagcttggt | gggcggggcc |
| 30241 | tgtttggggc | ggggcctgga | ggtgggcggg | ttttcttgag | ggccgggccc | ccagtcggag |
| 30301 | ccggcttcag | gaaagcaggt | ccgagagggg | tcacccggcc | cccggtgaga | attcgcagct |
| 30361 | caggccaggc | ctcttgttca | cctgttccgg | gcatgggggc | ggcctgcacc | ttgcgcctca |
| 30421 | cgttcctctt | ccctccaccc | gcctcctcca | gttctcttac | cacaatttcg | accggagccg |
| 30481 | gcacgacgac | gacgacatcc | gaggctgtgc | ggtgctggac | ctggcctccc | tgcagtgggt |
| 30541 | ggccatgcag | tgcgacacac | agctggactg | gatctgcaag | atccccagag | gttggccgga |
| 30601 | gtggcgctgg | gggacgcggg | atggagcgaa | gggtggcggg | gccaggaatt | ccagggaggg |
| 30661 | tctcctttcc | tcacaacgtc | tttgtccttg | ttcccctagg | tacggacgtg | cgggagcccg |
| 30721 | acgacagccc | tcaaggtgag | caccccccag | cccatcccgc | tacccatcgc | gtgggagagg |
| 30781 | gcgtcaactc | tggggtaagt | cccgagaggc | ctgaatgaaa | tggaggggat | gaggagttcc |
| 30841 | ggtcgagaga | agggggacgg | ggttggctcg | gacggaggca | gccacagccc | tgtttgcttc |
| 30901 | cgtgtgggag | gcgaggcaag | cctggggcct | ggggcctggc | acggtgcctg | cctgggtccg |
| 30961 | gtgtgcctgc | aggctcgcct | gctgccgcct | ggctctgccc | cggccctgcc | gcgcccgcct |
| 31021 | ccggggcctg | gcgtgcagcc | tctgcctggc | cgccgctccc | acgcccgcgc | tgctcctgca |
| 31081 | ggccgacggg | aatggctgcg | cttccaggag | gccgagtaca | agttctttga | gcaccactcc |
| 31141 | acgtgggcgc | aggcgcagcg | catctgcacg | tggttccagg | ccgagctgac | ctcggtgcac |
| 31201 | agccaggcgg | agctagactt | cctgagccac | aacttgcaga | aggtgggcat | tggattgagc |
| 31261 | agggggctgc | aggctggggg | agggcaggcc | cgggatgagg | gcagggggct | gcctacccac |
| 31321 | acctgcccgc | ctggcttctc | cagttctccc | gggcccagga | gcagcactgg | tggatcggcc |
| 31381 | tgcacacctc | tgagagcgat | gggcgcttca | ggtaggaacc | caggcaggca | gcagatgtgg |
| 31441 | aagggggctg | gctgtccaca | cacggagcac | agaggcagac | ttgcatttga | atccagccct |
| 31501 | gccccttcct | tgctggctgt | gtggccttga | gcaagatact | taacctttct | gggccttggt |
| 31561 | tttctcatct | gaaaatggga | atatgaggcc | tgcctggtaa | gatttttgtg | aagattagaa |
| 31621 | ataacatatg | cgaagtgctt | acgcagcatc | cagcacatta | gttactaaat | aatggcagct |
| 31681 | cctgttacgt | ctggtgtcat | tcatcatgac | taaggccctc | tcggggaagt | gggcagaaca |
| 31741 | tatgatgatc | cgggggcctc | tttgtatttg | gtgcctactt | accatgtact | ggggggcttg |
| 31801 | aggcaggaag | gaccaggagc | acctgggact | cagaatttga | gcttgtcttt | ttgcccttgg |
| 31861 | gcttcctgcc | ctggagtcct | caggcttctt | ttctggagca | gcaagtgtcc | agggacagct |
| 31921 | ggggccttat | ctagaggcac | caggtccttg | ggtctttgac | tgccctgagc | tcagtagctc |
| 31981 | cacccctcag | cctacagcca | ctaaacccca | gaacctgggc | ccttccctgc | ccccattcct |
| 32041 | gcggccttca | ttgctgcatc | cggtggaggc | ccagctgggg | gccggtgctg | gagttacctc |
| 32101 | tgctccatgc | cattgcagat | ggacagatgg | ttccattata | aacttcatct | cctgggcacc |
| 32161 | aggcaaacct | cggcctgtcg | gcaaggacaa | gaagtgcgtg | tacatgacag | ccagccgagg |
| 32221 | tgagggcaag | gtgggggcag | agtgcgcagt | cagctgtgca | RagggttRat | gctgggcgag |
| 32281 | cctgggcaga | agtcatcagt | ctgttctcat | tctctttgtt | cccacaaccc | aataaaggct |
| 32341 | agactcagcg | aggggtggac | ctgtctccat | ggtccagaag | acttgaattc | ggagctggac |
| 32401 | acaggccctg | agcctctgcc | cagactcctc | tcagggcatg | aggctgagtg | tgctgccagc |
| 32461 | catgccctgc | cctgctctgc | cctgggggtg | actgccctcc | cctccccttt | ccgcagagga |
| 32521 | ctggggggac | cagaggtgcc | tgacagcctt | gccctacatc | tgcaagcgca | gcaacgtcac |
| 32581 | caaagaaacg | cagcccccag | acctgccaac | tacagccctg | gggggctgcc | cctctgactg |
| 32641 | gatccagttc | ctcaacaagg | taggaggtgg | caatggggca | cccaagggag | tcaggggaga |
| 32701 | ggacgtgaac | agggaaaggc | tgagcctcag | gagtcctggc | cttttggcag | caccaaactc |
| 32761 | atggcctctt | gcctgtaccc | accaacttct | ctttcacctg | gctccagtac | cagggccaga |
| 32821 | ctccttggaa | aatgactcac | tgtagtagca | ccttccaatc | ctccgggaag | caMggttgtc |
| 32881 | caaataattt | ggtgagtgtt | ctagatcagt | gattgccaaa | gatttttttt | tttttttttt |
| 32941 | gagaaggagt | ttcactcttg | tcacccaggc | tggagtgcag | tggcgcgatc | tcggctcact |
| 33001 | gcaacctctg | cttcctgggt | tcaaccaatt | ctcttgcctc | agcctcccga | gtagctggga |
| 33061 | ttacaggcac | ctgccaccac | ccctggctaa | tttttgtatt | tttagtagag | atggggtttc |
| 33121 | accatgttgg | ccaggctagt | ctcgaacccc | tgaactcagg | tgatcacctg | ccttggcctc |
| 33181 | ccaaagtgct | gggattacag | atgtgagcca | ccgcgcccgg | cctgattgcc | ggcctgatta |
| 33241 | tcaaagattt | tgataatgaa | ctcctattag | taaaacaaaa | tattgagctt | acatgaccaa |
| 33301 | actatgtgtg | tgtacattta | acttatatat | gtatatgtgt | gtgtaaataa | aacctacttc |
| 33361 | catactaata | tgactaatat | tatatatagt | aaaatatgta | taagaacaaa | agagtataaa |
| 33421 | acattagcta | aagattttta | aatactttta | agtttaaaac | atttttttgg | ctgcgcgtgg |
| 33481 | tgggtcatgc | ctgtaatccc | agccctttgg | gaggctgagg | cgggtgaatc | acctgagatt |
| 33541 | aggagttcga | gaccagcctg | accaacatgg | agaaacccca | tctctactaa | aaatacaaaa |
| 33601 | ttagctgagt | gtggtggcac | atgcctgtaa | tcccagctac | tcgggaggat | gaggcaggag |
| 33661 | aattgcttga | atcctggagg | cggaagttgc | ggtgagtcaa | gatcgtgcca | ttgcactcca |
| 33721 | gcctgggcaa | caaaagcgaa | actccgtctc | aaaaaaataa | acaaagaaaa | aaaaaacaac |
| 33781 | aaaaaaaaca | cctttttttt | tttttttttt | ttttactagt | ggtatgaaaa | catttttttg |
| 33841 | tgctcactca | aaagtgatta | attggctggg | agaggtggct | cacacctgta | atcccagcac |
| 33901 | tttgggaggc | tgaggcagga | ggatgacttg | agcccaggag | ttcgagacca | gtctggacaa |
| 33961 | catggagaga | ccatgtctct | acaaaaaata | ggaaaattaa | ccaggtgtga | tggtgcacac |
| 34021 | ttgtagttcc | agctacccag | gaggtgggaa | gtgggaggat | ctcctgaacc | caggaggtag |
| 34081 | aggcttcaat | gagccatggt | catgccactg | tataccaccc | tgggtaatgg | agtgagaccc |
| 34141 | tttctccaaa | aaaaaaaaag | atcaattata | ataattattg | tttaatatgc | tcactttggc |
| 34201 | agcacatgta | caaaaagtga | aacaatacag | agaagattag | catggccctg | cacaaggatg |
| 34261 | ctatgcaaat | tcctgaaggg | tccatatttt | gcgtagttca | tgaaagttga | aggttgttca |
| 34321 | acaaaaaaat | tgtctaaata | attttaataa | gagcaatgtg | attacatgta | cttcacgttt |
| 34381 | ttaaaaaatt | ttagtctgac | actttatgac | tcatgactca | caggtctaac | gtgttgaaaa |
| 34441 | tcaaagttat | ttcaaaacat | gcatttcatg | gtggctatag | attaaatatg | cctcacaaag |
| 34501 | acaaatagat | tgaagtgaaa | aaagttcatc | catcatttct | aaataattct | ccccattttt |
| 34561 | agtccttcta | ccaattaaac | aaacattttt | gtttaaaaat | tggctagtag | gctgggacgc |
| 34621 | tgagcactgg | gaggatcact | tgaagccagg | agttcgagac | cagcctgggc | aatgtagcaa |
| 34681 | gaccctatct | agaaaattct | ttttccaagt | gttttgagtg | tgcaggtagg | aaagtttggg |
| 34741 | gcactgacat | taatcactat | ggatgcattt | ctcaacacgt | ttttacaaca | ccccctccat |
| 34801 | agtgtgagtt | tcttccccac | agtcgttatt | tttccatgca | caggctgatt | tattagacct |
| 34861 | gattagtcct | tcattgtttt | tacctggcga | ttgacaacga | gttcatacca | ggaagagaag |
| 34921 | gttgtcattt | tccttgttat | gtgccagggt | aattggcatt | aatctcaagc | catggcttct |
| 34981 | ttgcaggaca | ttttaagaac | acttagccct | gttgtgaaga | ttagattagt | taacactgaa |
| 35041 | taatataatc | cacaaatcct | tgtcatacac | aggaaggcag | ccagcctctt | agtgtcatgg |
| 35101 | atctcctgcc | ctcctcagaa | tattcaaaag | aacattacac | ggccaggcgc | tgtggctcat |
| 35161 | gcctRtaatc | ccagcacttt | gggaggccga | ggcgggtgga | tcacctgaga | tcaggagttt |
| 35221 | gagaccaacc | agggcaacat | cgcaaaaccc | tgtctctact | aaaaatacaa | aaaattatct |
| 35281 | gggcatggtg | gtggacgcct | gtaatctcac | ctactcggga | ggctgaggca | gcagaatcgc |
| 35341 | ttgaacctgg | gaggcagagg | ttgcagtgag | ctgagatcga | gccactgcac | tccagcctgg |
| 35401 | gcgagagatt | gagactctct | ctcaaaacaa | aacaaaacaa | aaaccacaMa | aaaaaaaaat |
| 35461 | tacacctctt | ctaccttaaa | aaattaaggc | atgtatatta | ttaaaacttt | ggaaaataca |
| 35521 | gaagagaaac | caaaactcac | ccaacactct | ctagagatca | cagctatcaa | tatttgttat |
| 35581 | tctccttcct | gcctttttcc | atggaattga | gctcatacta | aataatataa | tttcctaccc |
| 35641 | aactcttctt | tctctgtatc | atattttgag | catctctcat | gttataaagt | ttttccaaaa |
| 35701 | acttgttctt | tttccatcca | ctttttaaac | agaatgtatt | ttatatatgt | aataggtccc |
| 35761 | attcttaatc | atcattgtca | agataccagt | tactctcctg | ccctgtggaa | agttctgttt |
| 35821 | gtttttgttt | ttgagacagg | gtcttgctct | gtctcaaaaa | ctgctggagt | atggtggtac |
| 35881 | catcatggct | tacttactgc | agcctcgacc | tcctaggctt | aagtgatcct | cctgcctcag |
| 35941 | cctcctgagt | agctggcact | acaggcttat | gccaccatgc | ccagctaatt | tttctatttt |
| 36001 | ttttgtagaa | atggggtttt | gccatgttgt | ccaggctggt | tttgaacttc | tgggctcaag |
| 36061 | cgatcctcct | gccttggcct | tccaaagggc | agggattaca | ggtgtgcgcc | actgtgcctg |
| 36121 | gcctgtggaa | agctctggaa | gtatgctagg | gtgttcactc | cttcactgcc | tgccctcaca |
| 36181 | catcttgctt | tactagttct | gtgcccttct | ttgtgtccct | atagcatttg | gatgtaggta |
| 36241 | aagcatttat | ggcagctgtc | cccaaccttt | ttggcaccag | ggactggttt | cttggaagac |
| 36301 | aatttttcca | tgtactaggg | gaggggaggg | atggttttgg | gatgaaactg | ttccacctca |
| 36361 | gatcatcagg | cattagattc | tcataaggag | cacgcaacct | agatcccttg | catgtgcaat |
| 36421 | tcacggtagg | gttcgcactc | ctatgagaat | cttggctggg | cgcggtggct | cagcctataa |
| 36481 | tcccagcact | ttgggaggcc | taggtgggca | gattacctga | ggtcaggagt | ttgaaaccag |
| 36541 | cctggccaac | atggtgaaac | cctgtctcta | ctaaaaatac | aaaaattagc | caggtgtggt |
| 36601 | ggtgggcgcc | tgtaatccca | gctactcggg | aggctgaggc | aggagaatag | cttgaacctg |
| 36661 | ggaggcggag | gttgcagtga | gctgagattg | cgccattgca | ctccagcctg | ggcaacaaag |
| 36721 | caagactcca | tctcaacaac | aacaacaaca | acaacaacaa | caacaacaaa | gaatctaatg |
| 36781 | ccctgctgat | ccaacaggag | gtggagctca | ggctgtaatg | ccccttcgcc | ccKctgctca |
| 36841 | cctcctaaga | ggccggggac | ccctggtttg | tgggacagcc | tcttccacta | gctttaagct |
| 36901 | cctggagggc | agggccacta | gcacctgcca | taccccttaa | tacacttgca | cRtggcaggg |
| 36961 | atgccctcag | tgtcggctgg | actgagctga | ataccctccc | cctttagctg | tcacttcttg |
| 37021 | ctggtactgg | tgtgaccttg | ctgcttccaa | gtttatgatg | aattctactc | atgatttaag |
| 37081 | ttgggcctca | gcatggctct | gaaactactt | ctgtgaaacc | aaatccttga | actttgtgac |
| 37141 | agacctccaa | aaatctgcat | tatttctggg | cttacaattt | tttaccccaa | atctgccatc |
| 37201 | tgcatcagta | aggatggtgc | tggagctaat | tattaatagt | cacagctctc | actggttaag |
| 37261 | tgcctactgt | gtcatgtact | gttatcttta | atccttaaaa | tgatccccaa | agacaggctt |
| 37321 | tgtattacca | ctttacagaa | gagtaaattg | aggccataag | gttgtcagag | ctggaatctg |
| 37381 | acatctcccc | cttacctgaa | tcccacccct | gtctttatga | ctccaaagct | tccctacacc |
| 37441 | ctttcttttt | ttttcttttg | agacagagtt | tcgctcttgt | tgcccaggct | ggagtgtaat |
| 37501 | ggcgtgatct | cagctcactg | caacctccgc | ctcctgggtt | caagcgattc | tcctgcctca |
| 37561 | gcctcccaag | tagctgggat | tacaggcatg | tgccaccacg | cctggctaat | tttatatttt |
| 37621 | tagtagagat | ggggtttctc | catgttggtc | aggctggtct | cgaactccag | acctcaggtg |
| 37681 | atccacccgc | cttggcctcc | caaagtgctg | ggattacagg | tgtgagccac | cgtgcctggc |
| 37741 | caacaccctt | tattttccat | cacactgcct | ctgagcagaa | aatcattaga | tattcatttt |
| 37801 | cctaagaaag | agtcttcact | tgatgagaga | tttcctaccc | agggcttttg | tttgtttgtt |
| 37861 | tgtttggaaa | ccttatagag | ctaatgaaag | tgggaaaaga | gaaatgtaaa | attacagagt |
| 37921 | tagaagctgc | tgctaataga | tcattcaaca | atagagataa | aagYgtccta | gggctgggcg |
| 37981 | cggtgaaacc | ccgtctctac | taaaaataca | aaaaattagc | tgggcatggt | ggtgggcgcc |
| 38041 | tgcagtccca | gctactgggg | aggctgaggc | aggagaatgg | tgtgaaccct | ggaggtggag |
| 38101 | cttgcagtga | gccgagatgg | cgccactgca | ctccagcctg | ggagacagag | cgagactctg |
| 38161 | tcttgaaaaa | aaaaaaagtg | tcctagagat | gatcatgtaa | gttcaaaggg | caagataatg |
| 38221 | gatgtcagac | aacattcgga | agggtgttta | gcagaaagca | agatagatcc | ttgtccccat |
| 38281 | cttcctagtg | cttggcctag | gcactgttgt | catagccttt | gacggcccaa | agaaatcctc |
| 38341 | ccatttgaag | atcccctgga | gtgtgggcag | gtgtgtgtga | atctgcagtg | ccaagctggg |
| 38401 | aggtgctgga | ggctgccaag | cagacccagc | tgtcctggtt | ctcagatact | gttctctccc |
| 38461 | ggccctcaaa | ggccccccag | gacctgctgc | tgaggggcgc | acagtggcct | ttctgggttt |
| 38521 | gtggatgggg | tggtggggct | ggccattaca | tccccacctc | tgccccacag | tgttttcagg |
| 38581 | tccagggcca | ggaaccccag | agccgggtga | agtggtcaga | ggcacagttc | tcctgtgaac |
| 38641 | agcaagaggc | ccagctggtc | accatcacaa | accccttaga | gcaaggtagg | gccagcctat |
| 38701 | ggggagcccc | cagtatcctc | tgacactgtc | cccccaaata | gctatagcag | agtcactcac |
| 38761 | aactgtcttc | tggggaccat | agcattcatc | acagccagcc | tgcccaatgt | gacctttgac |
| 38821 | ctttggattg | gcctccatgc | ctcgcagagg | gacttccagt | gggtggagca | ggagcctttg |
| 38881 | atgtatgcca | actgggcacc | tggggagccc | tctggcccta | gccctgctcc | cagtggcaac |
| 38941 | aaaccggtga | ggatgccctc | cctgttcccc | tccgcaccgc | gctgccctaa | gcctgtgggg |
| 39001 | ctgcctttgc | cccagcatct | cttgccccag | ggtctccctt | cttccagcct | ctttgctcac |
| 39061 | attgcagcct | tctggggaca | gtagcacagg | gaggcagctg | ctggggctcc | cctgagcagc |
| 39121 | tccctccccc | cagaccagct | gtgcagtggt | cctgcacagc | ccctcagccc | acttcactgg |
| 39181 | ccgctgggac | gatcggagct | gcacggagga | gacccatggc | ttcatctgcc | agaagggcac |
| 39241 | gggtatgtgt | caccagtcac | ctgggaaacc | ccagtggggc | cctggcaccg | ggctggatgc |
| 39301 | tgggaccatg | ccaggaggaa | ggaatcatgg | tccctggcag | agcagggctc | tgcttgggaa |
| 39361 | gcagatgggc | agaaaggcaa | ggtgccaata | agatggaaaa | gcaatagcac | ccacaggtct |
| 39421 | caggaggaag | atttgggggt | atggagggct | cgttgggtcg | gggggagagc | tagacgctgg |
| 39481 | gctgcatgaa | gtgctggcac | gtgtggcgtc | aaggaccctg | aggttccagg | tccaagacac |
| 39541 | ctccagctcc | atgcgtaggt | ggcctctgcg | ccgggttcct | tgggtaggag | gaggtcaagg |
| 39601 | ccctcacctc | cctgtccatt | tgttagcaaa | catgtattat | taataacaac | aggRctgaca |
| 39661 | cctaccaagc | cttggtctgg | gccaagcgcc | gagtctggtg | gagaccKggt | ctttgcctgg |
| 39721 | tgtgcggcct | tctcctggcc | ttgtgcctgg | gaacggggtg | agggaagcag | agcccagcct |
| 39781 | gaaccctgac | cccttccctg | tagacccctc | cctgagcccg | tccccagcag | cgctgccccc |
| 39841 | cgccccgggc | actgagctct | cctacctcaa | cggcaccttc | cggctgcttc | agaagccgct |
| 39901 | gcgctggcac | gatgccctcc | tgctgtgtga | gagccgcaat | gccagcctgg | cctacgtgcc |
| 39961 | cgacccctac | acccaggcct | tcctcacgca | ggctgcccga | gggctgcgca | cgccgctctg |
| 40021 | gattgggctg | gctggcgagg | aggtgggctc | ccgacacttt | tgccctgggc | cYcagccttg |
| 40081 | cccgggttcc | cctccctgcc | ccttcctgcc | cccgccttat | ccgcacccta | tcctgactat |
| 40141 | tcccttcctc | ccaccccatg | gccccccatg | cccagggctc | tcggcggtac | tcctgggtct |
| 40201 | cagaggagcc | gctgaactac | gtgggctggc | aggacgggga | gccgcagcag | ccggggggct |
| 40261 | gtacctacgt | agatgtggac | ggggcctggc | gcaccaccag | ctgtgacacc | aagctgcagg |
| 40321 | gggctgtgtg | tggggttagc | agtggtgagt | gcccacctgc | cagggcgggg | gcatgggcaa |
| 40381 | gctgtcggtg | gccccgggga | gggtgctgag | ccacttctta | atcctgtacc | cccacagggc |
| 40441 | cccctcctcc | ccgaagaata | agctaccatg | gcagctgtcc | ccagggactg | gcagactccg |
| 40501 | cgtggattcc | cttccgggag | cactgctatt | ctttccacat | ggagctgctg | ctgggccaca |
| 40561 | aggaggcgcg | acagcgctgc | cagagaggtg | ggtgaccagg | ccagagccca | cacatggcgg |
| 40621 | gcaggtggca | cctcctctcg | tgggcactca | gacccatgag | tacatcccca | cactgctctc |
| 40681 | tacccatagg | cagcacttac | acaagatgcc | ctcgtgctct | cacatgtgga | gaccatacat |
| 40741 | gacactatat | gtgcatatgc | tgccccccca | cacacttgca | catgtgcaca | cacacacaca |
| 40801 | tgaatccaca | gactccacac | catcctctac | tcaggctgca | gagaagaggg | ctggagcagc |
| 40861 | tctgggggac | tctgcccccc | ccggagaccc | atccgccctg | acgtgggcct | tctttgcatc |
| 40921 | cagcgggtgg | ggccgtcctg | tctatcctgg | atgagatgga | gaatgtgttt | gtctgggagc |
| 40981 | acctgcagag | ctatgagggc | cagagtcggg | gcgcctggct | gggcatgaac | ttcaacccca |
| 41041 | aaggtgggtg | ccctgtgtgt | ggggtggaga | ggtcaagcct | caggctgtaa | ggggctgccc |
| 41101 | tccactttgc | cctgagcctt | gtcctggggc | ttgtcggggg | acagggagtc | ggttctagaa |
| 41161 | cacRcctgct | ctcctccacc | tactcctgcc | ccaccttgtc | cctgctccct | cagtgccttc |
| 41221 | tttcctgcag | gaggcactct | ggtctggcag | gacaacacag | ctgtgaacta | ctccaactgg |
| 41281 | gggcccccgg | gcttgggccc | cagcatgctg | agccacaaca | gctgctactg | gattcagagc |
| 41341 | aacagcgggc | tatggcgccc | cggcgcttgc | accaacatca | ccatgggtgt | cgtctgcaag |
| 41401 | cttcctcgtg | gtgagcgccg | ggcaggccca | cgctcagcct | ccttccaccc | cgtgccgctg |
| 41461 | gtcctgggct | ggggctgggg | gctgcttcac | aactgcaggg | ggcacagccg | atgggaaggc |
| 41521 | ctgaacagaa | cggactgcgg | gcagctggga | cccccgggat | aaattagtca | gtgggatgga |
| 41581 | ctggacctga | aacttatcat | ccaaactaga | ccacaaggct | cgggggtcag | caaccccctg |
| 41641 | tcactttgta | gtggtcaagg | gcatggacag | agttcaagtc | ctggccatgt | cacttatcag |
| 41701 | tggtgtgacc | ttgggcagag | tctgtggcct | cagtttcctc | gtatgtaaaa | tgggaataat |
| 41761 | agtagtYcca | gactgatgtg | gtggctcatg | cctgtaatcc | cagcactttg | ggaggctgag |
| 41821 | gtgggcggat | cacttgaggt | caggggttca | agaccagcct | ggccaacatg | gtgaaacccc |
| 41881 | gtctctacca | aaaagtacaa | aaattagctg | ggcgtggtag | cacatgcctg | tagtcccagc |
| 41941 | tacttaggag | gctgaggcat | gtgaattgct | tcaaccgtgg | aggtggaggc | tgcagtgagc |
| 42001 | cgagatcgca | ccactgcatt | ccagcctggg | caacacagca | agaccctgtc | tccaaaaaaa |
| 42061 | aaaaaaaaaa | aaaaaagtcc | cgacctccta | gagtgctagt | gaagctagta | aagatgaggt |
| 42121 | gagttaggga | aggggctgac | acccaggcct | cccacgaggg | ctgggctgag | acctagctga |
| 42181 | gtgcagcatg | gcgttttccc | tttctggggg | aacctgaagt | cttgtctctt | gggcctgtgc |
| 42241 | ccccacagta | accagaggta | ggatgggagg | gggaactaga | gcctctttct | ccccagacct |
| 42301 | cccggcccag | gcctgtgtgc | tttgtatgtt | tacttaagtg | attattacga | tgatcactgc |
| 42361 | tattattaac | tggccccctc | ctcttgccca | cagctgagca | gagcagcttc | tccccatcag |
| 42421 | gtgagtgaaa | ggcaatgccc | ccaggtgggc | aggcaggaag | cactgctggg | cctaacgccc |
| 42481 | acttggcctt | tcacgcccac | tcgccttggc | agcgcttcca | gagaacccag | cggccctggt |
| 42541 | ggtggtgctg | atggcggtgc | tgctgctcct | ggccttgctg | accgcagccc | tcatccttta |
| 42601 | ccggaggcgc | cagagcatcg | agcgcggggc | ctttgagggt | gcccgctaca | gccgcagcag |
| 42661 | ctccagcccc | accgaggcca | ctgagaagaa | catcctggtg | tcagacatgg | aaatgaatga |
| 42721 | gcaRcaagaa | tagagccagg | cgcgtgggca | gggccagggc | gggaggagct | ggggagctgg |
| 42781 | ggccctgggt | cagtctggcc | ccccaccagc | tgcctgtcca | gttggcctat | ggaagggtgc |
| 42841 | ccttgggagt | cgctgttggg | agccggagct | gggcagagcc | tgggctggtg | gggtgccacc |
| 42901 | ctcccacaag | ggctgggctg | agacccagct | gagtgcagcg | tggcgtttcc | ctttctgggg |
| 42961 | gggcctgagg | tcttgtcacc | tggtcctgtg | cccccacagg | aaccagaggt | aggatgggag |
| 43021 | ggggaacgag | agcctctttc | tccccagagc | ccccggccca | ggcctgttga | tccgcgcccc |
| 43081 | aggaccccct | tctttgcaga | gcccgaggag | cctcccctgt | cccctcgggc | agatctgtKg |
| 43141 | tgtctctctt | cccacctggc | agcctcagct | ctgtgcccct | caccctgctc | cctctcgccc |
| 43201 | cttctctccc | accccttcct | tctgagccgg | gccctgggga | ttggggagcc | ctcttgttcc |
| 43261 | tgatgagggt | cagctgaggg | ggctgagcat | ccatcactcc | tgtgcctgct | ggggtggctg |
| 43321 | tggggcgtgg | caggaggggc | ctaggtgggt | tgggcctgag | aaccagggca | cgggtgtggt |
| 43381 | gtctgctggg | ctggagataa | gactggggag | agacacccca | acctcccagg | gtgggagctg |
| 43441 | ggccgggctg | ggatgtcatc | tcctgccggg | cgggggaggg | ctctgcccct | ggaagagtcc |
| 43501 | cctgtgggga | ccaaaataag | ttccctaaca | tctccagctc | ctggctctgg | tttggagcaa |
| 43561 | ggggaagggt | tgccagagtc | ctgggggccc | cagaggagca | ggagtctggg | agggcccaga |
| 43621 | gttcaccctc | tagtggatcc | aggaggagca | gcacccgagc | cctggagtgg | cccagtaccc |
| 43681 | ttccaagagg | ccacagtccc | agccaggaca | aagtatgcgg | cccatcctgg | tgcgacagcg |
| 43741 | tgggacaatg | tgaacatgga | ctcgaagaca | tggccctttc | tctgtagttg | attttttaaa |
| 43801 | tgtgccatta | ttgtttttaa | aaaaaaagga | aaaaagaaaa | gcaaacaaat | aaaacacctt |
| 43861 | taagaggctt | gaaagagaag | gttctgcctt | gtcctgtggt | gagcctgctt | cttcacgtct |
| 43921 | gtcccttcgg | agataactgt | tgttttggag | cttgcaccac | aggtgttttc | tgcagctggc |
| 43981 | cattttgcgt | ctgtctctgg | gcctctaggg | gacactctca | cccccgatct | ttaggagctc |
| 44041 | acccctctgg | gcctgcgcct | ccctctctct | gttcctgggg | gcttgaatgt | gggtgagaac |
| 44101 | tcaggagcca | ggccatctgc | tgctgagcag | ccagccgtgc | aagcttgggc | aacttagcac |
| 44161 | acctctcaac | atgtgcacag | tggggagaac | cggacccacc | tcctagggtt | gttttaagga |
| 44221 | tatgtgagga | tcaagggagc | tcatctgtgc | agagcactta | gaacagtgcc | tctgggctct |
| 44281 | gcaagcgcta | ggcgttactg | ccgtttctgg | gctgcggctc | caggggagca | cttgatgtgt |
| 44341 | ccaggccggc | cctgggtggg | ggctccacat | caccgtcctt | ccaaggcagc | catcatacct |
| 44401 | gccctctaac | agcctgaaag | ttcacatgtg | tccttaggga | gcctgggctc | tgaaaccaca |
| 44461 | gaaggcagaa | tggacggcag | gacctttact | cacctgcttg | ttcattcagc | cggtatttga |
| 44521 | gctggagatg | ccaacaaagg | aaggttaacc | aacgccaatc | gggcgaggca | gggggaagct |
| 44581 | gctaatgtgg | cctgtgtgtc | tggggagggt | ctcactagag | ggagtgagat | tgactgcagc |
| 44641 | ctgaaggagg | tggcggagtg | agcaatgcaa | atatctgggc | cagggctagg | ggggcatccc |
| 44701 | tggtgcttta | ggggaccagc | agggaggcga | ggtgggctga | ggctgagatg | agtcaggcag |
| 44761 | ggccagattg | tccaaggccc | tgaaggcagt | ggtagaatgt | agcattattt | tcaggatagt |
| 44821 | gggaagtcaa | tgggaatggc | taatcaggct | gaccttttaa | caagatcatt | ctggctagga |
| 44881 | gttgagaaat | agacaatgga | tgggaaacgg | gggagtgaaa | gcaggaaggc | tggtgcggaa |
| 44941 | gctcttgtaa | taattcaggc | acaacaagac | ggtggtttgt | tgggatcagg | actgcagtag |
| 45001 | tggtcagatt | ctgtgtgtgt | gtgtgtgtgt | gtgtgtgtgt | gtgtgcgcgc | gcgtgcgtgt |
| 45061 | gtgtgtgttt | tgccctgttc | atgatattta | aaataagttt | ccacttttat | gaatagacaa |
| 45121 | tgcagtcacg | cagtctgcat | tgcgtgggat | acctaaatga | tatcccaaga | tagatgttgc |
| 45181 | gcaagtcctg | cccctgcccg | tcgccctcca | gcctataccc | tcctgccctg | tcagtttcct |
| 45241 | gtctttccag | tgtttctttg | tgaaagcaaa | tgaacgccag | attggactga | agggatggcg |
| 45301 | gatgcccttg | ctggggatgg | gatctgcctg | gggaaaggca | gtgggagtca | ggaagcactc |
| 45361 | caagcttccc | gcctggactg | cagggtgaat | ggggtgctgt | ttcctgagac | aggagcccta |
| 45421 | gggagagccc | atgagctgct | ggcctgagtc | caggactacg | gtggagccca | ggggaccccc |
| 45481 | tccagcaatc | ctctctgtgc | cgccccagca | acagcaggac | ccatggaagg | cgcctaatca |
| 45541 | ctcaggggag | ggagtcccag | tcctgtggga | ggttcaccct | ggagtgagtt | gctatctagc |
| 45601 | cctgcagccc | aaacagctcc | ctttgtgagt | taagtgccct | gccactgccc | acagtgagag |
| 45661 | gtgaagccag | ctggacttcc | taggttgagt | ggggacttgg | agaacttttc | ttacaagagg |
| 45721 | tttgtaaaat | cagtgctctg | taaaaatgca | ccaattggtg | ctctgtagct | agctagaggt |
| 45781 | ttgtaaaatg | caccaatcag | tgctctgtaa | aaatgcacta | atcagcactc | tgtacctggc |
| 45841 | tagaggtttg | taaaatggac | caatcagcac | tctgtaaaat | ggaccaatca | gcaggacatg |
| 45901 | ggcggggaca | aataagggaa | taaaagctgt | gtcctcccca | cccaccccac | ccctagcagc |
| 45961 | aacccgctca | ggtcctcttc | catactgtgg | aagctttgtt | ctttcactct | tcacaataaa |
| 46021 | tcttgctgct | gctcctcact | ctttgggtcc | gtgccacttt | taagagctgt | aacactaggc |
| 46081 | caggcatggt | ggctcacgcc | tgtaatccca | gcactttggg | aggccgagac | aggcagatca |
| 46141 | tgaggtcagg | agatcaagac | tatcctggct | aacatggtga | aaccccatct | ctattaaaaa |
| 46201 | tacaaaaaat | taactgggcg | tggtgacagg | cgtctgtagt | cccagctact | cgggaggctg |
| 46261 | aggcaggaga | atggcgtgaa | cccaggaggc | ggagcttgca | gtgagccgag | attgtgccac |
| 46321 | tgcactccag | cctgggcgac | agagcgagac | tctgtctcaa | aaacaaaaaa | aaagagctgt |
| 46381 | aacactcacc | gtgaaggtcc | gaggcttcat | tcttgaggtc | agtgagacca | cgaacccact |
| 46441 | ggaaggaacc | aactccagac | acaacaccac | aagaaacatt | gggggagagg | ccccccaaga |
| 46501 | ccctagtgac | tctaacctcg | ggagagaaga | aaggtcaagg | agtgaggtcc | ggatcactag |
| 46561 | cttgaggggt | gggagggagc | agcggaggga | cggaggaccc | cagtggccag | agctgccctc |
| 46621 | gtcagtggct | ggtgcctgct | ctcctcacgt | ctctctgcct | tgctcaggtg | acgagtctcc |
| 46681 | ccagggccct | gcacccacct | gcaagcattt | cagacagcgt | cacgggtgcc | tggaggctac |
| 46741 | ccctagacct | cgttctaagc | ctgcccagag | atgactaagg | gaggctcctg | catctctctg |
| 46801 | aaggggcagg | aggcaggctc | tggatgccgg | ccactcaccc | tctggctcag | gatagccctg |
| 46861 | gtggcagtct | ccttcctttc | tgctcatttc | atttcaatac | atcaagtacc | tcctctacat |
| 46921 | accaggctct | ttgctgggtg | gcagatgccc | ccaagcctat | cctggcaggc | ctgggcaatc |
| 46981 | agcactgtat | gccaggcgct | aggcccagcc | ttcacttgca | gggttaaccc | gccccgctct |
| 47041 | ggccacctgg | atccgtgaat | gaatgggaga | atgcggctgc | tctccagaga | acagcattct |
| 47101 | ctcaaccgct | tcttgtcgcg | ccagagatgg | cctcctcgaa | ggtgtgctgg | cagagctggc |
| 47161 | ctggcccagt | gggcagccct | gtgaggccgc | aggggtgagg | agccgccagc | cacaccacat |
| 47221 | ggctctcctg | gaacggagcc | Rcctgagccc | ggccaagccg | cctcgagcac | cagggcttga |
| 47281 | gtccattgca | ggcacaaggc | aaagaattgc | gacttgaggg | aggacccgga | ggaaggcagg |
| 47341 | ctggagggag | ggagggcggg | caaaggacag | cagggaaatg | aagggggctg | tgaaagacca |
| 47401 | gcaccagatg | aaagtaccga | gatgttccgg | acaggcctga | ttttctttat | cttcggggct |
| 47461 | gggactgcca | agaggccccg | gggaaagcct | gcaggcttca | ctcccaccag | aaccagcacc |
| 47521 | atttgaggga | cagatttcaa | tccttccttg | gccagacaag | caggcgggat | gccagcgctg |
| 47581 | gagaaagggg | aggaggagga | gcggtgggca | ggcctttcct | gctggaggtg | gacacgctgg |
| 47641 | gggcgctgag | gttcaaggcc | acacagtgag | gagtgcagaa | ccttaactgt | ctccttgcat |
| 47701 | cggctgatgg | cagagcttta | gttatagcca | tttcagggaa | atcctggtca | gttaaggttt |
| 47761 | tctagtgcag | ttgggatttt | cgcctatatt | tccaggtgcc | tctaaggtcc | tatggagcgt |
| 47821 | ccaggtttca | cagctggggg | agggacatct | ggttcccggt | catttatgtc | cccgctggct |
| 47881 | tatgcagggg | tgtcctgccc | ttctgcatct | ccctcaggga | cttggggaac | cttggatggt |
| 47941 | gtctttcctc | atactggggc | tgcagggggc | tcagaacact | gcctctgacc | cactattcct |
| 48001 | ccatgacaat | gataataaca | aaataggtat | tatttactat | gtacacgccc | tgttctaact |
| 48061 | gcttaacacg | tattaactca | cttaatcttc | ccaatacaac | ataaggtagg | tgctactgtc |
| 48121 | cccctcttac | agatgagaaa | gctaaggcac | aaaggtgaag | aacttgccat | gccacctctc |
| 48181 | ctccctgcac | tggaaaacgt | gttgtggggt | gggcaacagg | gctacagaga | gcccctcgag |
| 48241 | atcttgtagt | caggcctggg | gactaaagaa | ctttgtccca | acctctcatc | tctggagagt |
| 48301 | tttagggcta | ttttgagact | tgagggctaa | gaatgacctc | tttccttctg | agcctccacg |
| 48361 | gcctcccttc | ccaaagcagt | ggcggaggga | cacctgggtc | tcctgggctg | tggggtacaa |
| 48421 | tgcatagaaa | taaagtgggg | ctcggttgac | gtattagaca | tttataccat | tggggatgct |
| 48481 | ttattttata | ttttagcatt | atttattttt | attttttatt | tttctgagac | ggagtcttgc |
| 48541 | tctgtcaccc | aggctggagt | gcagtggcgc | gatctcagct | cactgcctcc | cgggcttcag |
| 48601 | cgattctcct | gcgtcagcct | cctgagtagc | tgagactaca | ggcaccctcc | accaggcttg |
| 48661 | gctaattatt | tttttatttt | tagtagagac | caggtttgac | catgttggcc | aggctggtca |
| 48721 | tgaactcctg | acctcaagtg | atccgcccac | ctcggcctcc | caaagtgcta | ggactatagg |
| 48781 | cgtgagccac | cgcacctggc | ctcatcactg | ccttttaata | aatgaggctg | tcctcttgga |
| 48841 | atctcaagcc | atgaaaagcc | ccatatggca | ggcagactcc | tgccaggaaa | tcaaattgat |
| 48901 | agtgtgggta | gtccccagct | cctcaatgtc | taccccaccc | tggacctcat | gggaagcatt |
| 48961 | cctggttggg | taaatccctg | aaacagtttt | ggccgggcgc | cgtggtttac | gcctgtaatc |
| 49021 | ccagcacttt | gggaggctga | ggtgggcgga | tcatgaggtc | aggagatcga | gaccatcctg |
| 49081 | gccaacatgg | tgaaaccctg | tctctactaa | aaacacaaaa | attagctggg | tatggtggcg |
| 49141 | aatgcctgta | atcccagcta | ctcgggtggc | tgaggcatga | gaatcgcttg | aacccaggag |
| 49201 | acagaggttg | cagtgagctg | agatcatgcc | actgcactcc | agcctgatga | cagagcgaga |
| 49261 | ctccatctca | aaaaaaaaaa | aaaaaaaaaa | aaagagtaca | gtttttcaaa | aacctgattt |
| 49321 | tctgatgtaa | acctgtgatg | gtctcctcag | ctgcctcaaa | cactgtcaca | tcctactgtg |
| 49381 | gattcaagct | gtcaggatgt | ttacacggcc | acctcaaatt | ccattccgct | ctctacaaaa |
| 49441 | acttattttt | agacgtgcac | tgtcccttct | tatacattcc | tcagtactgg | atcctggaca |
| 49501 | aatctttagt | caaccttaaa | gtttagttca | aaaggctcat | tttctcatag | agtttgaggc |
| 49561 | catttttgtg | agtggaggaa | cttgaggttt | taggaatttt | atttaaatca | tggagatatt |
| 49621 | ttcgactcga | atggctctta | actttgatct | tgggtctagg | ctgcctggtt | gaaactacta |
| 49681 | ctggttttct | ttcttttttt | ttttttgaga | tggagtttcR | ctgtgttgcc | caagctggag |
| 49741 | tgcgatggcg | caatctcggc | tcactgcaac | ctccgcctcc | tgggttcaag | caattctctt |
| 49801 | gcctcggcct | cccaagtagc | tgggtttaca | ggcatgtgcc | actatgcctg | gctaattctt |
| 49861 | tttgtgttta | gtagagttgg | gttttcgcca | tgttggtcag | gctggtctag | aactcccgac |
| 49921 | ctcaggtgat | ccacccacct | ctgcctccca | gtgctgggat | tacaggcgtg | agccactgtg |
| 49981 | cccagcgaac | ctactggttg | gttttcaaga | gttgcatcac | tttaactgga | acataNcgga |
| 50041 | agagtgggac | cagattttaa | ggtgagtcaa | gcctggcctt | cctaaaaccc | tattgactcc |
| 50101 | ttaattcagc | tcagggcacc | cctttgccgc | aaaaatctca | tcctttgaga | cccctaggag |
| 50161 | agggtgagtc | ggggaagcag | gggcctctcc | tagtctgaga | agtcttaaag | gcagggggga |
| 50221 | gaagtgccgc | ctcctcattt | catctttctc | tctcgttctc | ctctctctgc | cctttctctg |
| 50281 | tccctccctc | cattttttag | aaaatctata | gaggcaatga | gctttagcaa | atttatccag |
| 50341 | ttgtgtaaat | attgccacaa | agcagcttta | taacacctca | ctcaccccca | atagtcccct |
| 50401 | ggaccctatt | tgcagtcaat | ccctgctcct | ttcccagccc | ccagcaacta | ctgatctgct |
| 50461 | tgctgtctct | ctagttttgc | cttttctaga | aattgtgtaa | aatgaatcat | atactatgta |
| 50521 | gtgtttggtg | tctggctttt | ctcacttagc | ataacatttt | tgcatgtacc | gtagtttgtc |
| 50581 | ccctccatct | tttattatta | ttattattat | ttgtgacagg | gtcttgtagc | cttgtctaca |
| 50641 | aggctgcagt | acagtggtgc | gatcatggct | cactgcaacc | ttgaccccct | aggctcgtgc |
| 50701 | gatcctccta | cctcagcctc | ccgagtagct | gggcccacga | gggtatgcca | ctacacatgg |
| 50761 | caaattttta | aatttttttt | gtagagacag | ggtctcgcta | tggcccaggc | tgatttcaaa |
| 50821 | cttctgggct | caagcRgtct | acctgccttg | gcctcccaaa | gtgctgagct | tataggcatg |
| 50881 | agccaccata | actggccctt | ttatttttat | ttatttattt | ttgaggtgga | gtcttgcttt |
| 50941 | gttgcccagg | ctggagtgca | gtggtgcgat | ctcagcttac | tgcaggggtt | caagtgaatc |
| 51001 | tcatgcctca | gcctcccaag | tagctgggac | tacagatgtc | caccaccacg | cccagctaat |
| 51061 | tttttttttt | tttttggtat | ttttagtaga | gacagggttt | tgccacgttg | gccaggctgg |
| 51121 | cctccaattc | ctgacctcaa | gtgatccatc | ctcctcggct | tcccaaagtg | ctgggattac |
| 51181 | aggtgtgagc | caccatgtcc | agctctttta | ttgttttttg | gcttaaaaaa | caattttttt |
| 51241 | taaacaaaaa | cacacacaca | cacacacgta | ctcaaacaca | ctcgcacaca | caattttctc |
| 51301 | actaaccctc | tgaccttcca | ccccaagtcc | tgacttggtc | tttgggccct | aagatgcaat |
| 51361 | caggaaaaaa | agtgtggagt | ccacacccca | gttgggtcct | aagtgggtac | ctacaggggc |
| 51421 | agccaccatc | cagcctgccc | cagcccatgg | gggcccagga | tgtagcatcc | ccagtgatgg |
| 51481 | tgggagcaag | ggagaagtag | aggtgagatg | tgggctgtga | catgtgcttc | cctctccctc |
| 51541 | cagcttgaga | cccttggcct | ctgaccaagg | caagtgaaat | aaacatgaac | gaagctggga |
| 51601 | gaataatgtc | agtttactga | atgaatacat | ggctcgagtc | cattcagggt | ggagtgaggc |
| 51661 | ctggcaggtg | ccctcagcag | tgggacccca | ggccactgga | cctggcaggg | cttctgggct |
| 51721 | aagggggcag | caccaggcca | gcctgccagg | ggctccacag | tcccacgctg | cttgacctgt |
| 51781 | gctgtctggg | actagactgg | tcgctgtggc | tgcccataga | tgctcaagcc | agaccccaaa |
| 51841 | agagagtggc | acRaggtgaa | aatctaactg | tgaacgcttt | ggtttcagtt | tcagtaaaga |
| 51901 | ggaggaggga | gggaggcact | tggggacgta | gaaagaaggg | ctggcRggag | aggtctccgc |
| 51961 | cgagctccac | agttggcttc | cttgctagac | gacctggctt | tgagaaatgc | tgccttcatt |
| 52021 | tccatctccc | aactccgaat | ctggaaggca | agaaggtgtt | tcaggctgga | gggccgcagc |
| 52081 | agaccgtggg | tctgttacag | ggggcacggc | actgctgctt | catcttctcc | caccccatcc |
| 52141 | ctgaggccca | gccacccaca | tggccacagt | gcctggaacc | gtgtggggaa | cagagacctg |
| 52201 | cagtctgggg | ccggggaatc | cacagactgc | cccattgggg | tttcagccac | accagcttcc |
| 52261 | cccaaagaca | gcatcttggt | catctccacg | tccccacata | gcctggtgca | gaacttgttt |
| 52321 | tcaggctgaa | tagctgcagg | ggccattgct | tcctctcagg | gtaaggcccc | acttgaaccc |
| 52381 | ccgcttccta | atttggcagg | gaagggggaa | cggacccagg | tgtttcagga | gtctttcaga |
| 52441 | aatcaatggc | tggatgcaga | gggacaagtg | ataggacggg | gaaagggagg | gagagcaaga |
| 52501 | ggcactggga | cgcagtgcag | tcccagtgga | ggaggtggag | ttggtggggg | tgggtggtct |
| 52561 | cctttattct | tcggggaaac | tcttgtctca | atatgtttct | ttcccccttg | gcttgagtga |
| 52621 | atgctctctg | acaatttaga | aacagtttaa | gccaggcgtg | gtggctcacg | cctgtaatcc |
| 52681 | cagcgctttg | gaaggccaag | gggcgggggg | cggggggcgg | cgaatcactt | gaggtcagga |
| 52741 | gttcgagacc | agcctggcca | acatagcaaa | accccatctc | tacttaaaat | acaaaaaatt |
| 52801 | agccaggggt | ggtggcgggt | gtctgtaatc | ccagctgctt | aggaggttga | ggcaggagaa |
| 52861 | tcgcttgaac | ttgggaggca | gaggctgcag | tgagccgaga | tcgtgccact | gcacttcagc |
| 52921 | ctgggtgaca | gagcgagact | ccatctcaaa | aagaaaaata | aaaaaagaaa | aaagaaaaga |
| 52981 | aagaaagttt | ctgcctgttg | tggaagaaac | tgttgcacgg | agcaggagca | actgacatcc |
| 53041 | cctcaatcct | acctggaccc | tctcaccctc | tcagttctac | ccaaggaagc | ccaagcccct |
| 53101 | gagctgcccc | catcccctcg | tccatcctgc | gctttctcca | aaagccagta | gccttgactg |
| 53161 | ggtgtttggg | gaggtgctct | ggcccagcct | ttggccaagg | gccctcacac | ccgcccctgt |
| 53221 | tcctgcccct | gtcctcatct | gtttgcatat | ctgcaggagg | tattgccccc | actaccgacc |
| 53281 | caccacccct | tgtcttggtg | acactgggaa | gttatttgtg | ccactgggaa | gcagctgtcc |
| 53341 | tggtgctggt | tcctgggcac | acccagggac | tctccccagc | tgccctaacc | tcggtttcac |
| 53401 | ttgtggcatt | tgtgtgaggc | tttggttctc | acgtgcaagg | gaggggggcc | tggtcagcag |
| 53461 | gctggcatgg | gcaccacaga | gtcacaaaga | ggctggcacc | tctctgtgct | acctggtgtg |
| 53521 | ccctgatgtt | ttgcctcttt | tcctcctgtg | tacctcaacc | gagctagggc | agcaggtcac |
| 53581 | agctgtgact | cacctccggc | aacccatatt | aacaaagtgc | tgccaaccag | gcgagtcatg |
| 53641 | ccaggatgcc | aggggaccaa | gggagtgtgg | cctatggggg | aggctgggtg | cccatggctc |
| 53701 | tgccaatggc | taccctggct | cttgtcttga | gaacggctct | ggcgggaagc | caggccaagg |
| 53761 | ctgcgactgg | tcagcacccc | aggatggtcc | ctctgtgctg | ggttctcctc | ggcctcggct |
| 53821 | gtgcccaccc | gtgtctgggg | tgtgtgctga | gctgggggca | ggtggagatg | gcctgggcct |
| 53881 | ccaggggaga | actggctgtc | tccaccttgg | ggccgagagc | ggcaggggag | ggttggtgat |
| 53941 | ggggtggatc | ctccagcggg | tcccagttcc | cggaggaagg | atgacaaatg | agtgtgggtc |
| 54001 | gctccctctg | tgggaccatt | tgcctcggag | cagcagggaa | gggcggcagg | ttccctgcct |
| 54061 | cccgtggcga | ctcttttcgg | agcaccaccc | tagggatgtt | cttctgccct | ccggagacgt |
| 54121 | cggtcttccc | tggggccggg | gctcaaggcc | agggtgcccc | aagcccaggc | tccagggtgt |
| 54181 | gcggggaaag | gctcggtcct | gctcggcgcg | gctcgccctg | ggcactaggg | gtcgcccctg |
| 54241 | cccagcggaa | gccacgcggg | cgcgcccggt | cgccgcaggt | ttccccggac | cgggaaaagc |
| 54301 | gaggcgtctc | cggggcgggg | ctggggaggg | ggcgcctggg | cgcggcgccg | ggaccctgct |
| 54361 | gccctgtcct | cccggccgcg | gctggacgga | gcgcggggcc | gcagcaacca | ggccgggctc |
| 54421 | cccgtcgcgc | cccttccgga | gcgggaggcc | ctcgccgagg | cggaggctgg | gacgcgcggg |
| 54481 | gcgggcggcg | gcggggagcc | gcggggccga | ggcgcagggc | gttttccccg | gaggtgcggc |
| 54541 | gtgcgcccgc | gacacaaagg | tcttttaaag | acctcgcagc | gcccttcccg | gctgcggagg |
| 54601 | ctggaggccc | gggggggcga | ggccggggtc | gggggtggcc | gcgcgagggg | gcgctgggat |
| 54661 | cggccagacc | ccgcggttgc | gcccgggctg | gggctcccgg | gctccttttg | tggaaaagag |
| 54721 | cggaaagcag | ggccctctcc | gggtctctga | aaatggtcag | ttcggcttcg | cagggctgcg |
| 54781 | tgcccggaat | aataaattgg | cttctctagg | gtcgtaatct | gggccccacc | ccgagcagct |
| 54841 | ttgagctggt | tcgcgccttc | caggcaggac | tactgggagt | tctcaaaagc | acccccgcag |
| 54901 | tcggctgtgt | caggatcacc | tgggtccggt | gtgaacacga | ttccagggcc | ccaccccatc |
| 54961 | gcctgaagcg | gaatctctgg | gcggggccca | ggaaaccgag | tttttggtga | gttcccgggt |
| 55021 | gattctgatg | gcaaagccca | tgtgggagcc | actgttctaa | gggcttttgg | acaggcagtt |
| 55081 | gtgaaatttt | cccccagaag | aatctctggg | gacccagttg | tcacacaccg | ctggattcac |
| 55141 | tgcggtgttg | attctgcctc | ctgatgaagt | tcttcccgag | tctgacttcg | agcccgcctg |
| 55201 | ctttattaag | cacgtttttc | tccagtcgtt | tttttttttt | tttaatggaa | aaaggaacag |
| 55261 | ggagaaggct | tttctcgaac | ctgtttgcag | agagccgtct | tgcccgcttt | gagggccctt |
| 55321 | tcttgggaga | cctgtttgct | ttgttcaacc | tctgaattca | gcagacccca | aatccaccaa |
| 55381 | ggccactagc | ttgtgagtga | atgtggtccc | ttcaggtaag | ggccagacag | cccctgccag |
| 55441 | ggcttgggga | gggttttcct | agaggaatcc | tggcagtaaa | aaaaccaacc | cccaaactct |
| 55501 | ccaagtgctg | gacgctggag | aatcccagca | aggaaaagga | acaaatctta | tctcatgccg |
| 55561 | gaagataacc | tcttcccttt | gttggcgcct | ttccttgcgt | tcaggatgaa | ttaaaattct |
| 55621 | tatctgctct | gcatccagtg | aacttggcct | cgccttcctg | attgtttgct | gcggctgact |
| 55681 | ttcccaagct | ttgcggggtt | atttttgcct | ctgggtggtg | gtcattcctt | ccttccatgg |
| 55741 | catttggttc | cacacagtca | cagaaaaaga | gtaatttagc | aaacaccctc | aaaatggcag |
| 55801 | gactggcccc | caaaacctac | tttcattttc | ctctgttctg | ggttgtgggt | aatttcttga |
| 55861 | caactacaag | aaagaagaca | atgagaaatg | aattgttttt | tccaatacga | ttgtgaacag |
| 55921 | atgtataacc | tcctagtcgc | agcaacgttc | taggaatccc | ttacaccact | ttaaaggggc |
| 55981 | aggaaaatgg | gcagaggtgc | tgagggctgg | gtgagtgcta | agcattggca | gggctcagcc |
| 56041 | tcatcctgac | gagagctgca | gccataacct | gggtcacctt | tgctgtctcg | ggcttctaaa |
| 56101 | tttggcagcg | agaacagact | cgcttccttt | gagcaattga | tctgaatagc | agaatcccat |
| 56161 | tctgatggtc | cgaatcacat | ttacaggctg | tgggggtgaa | ggccgtgcta | attggccact |
| 56221 | tgcctcacca | ctggctttga | aacaatcgct | tgtattctct | gcgcggcagg | aagcagcaga |
| 56281 | cccatcaagg | ccctgggctc | actctttgtt | ctgactttat | ggcattttat | gccacaataa |
| 56341 | gaaacactgt | gtactccgtg | ggagcccatt | gtgccaggct | gcaaggatgt | gggctccggt |
| 56401 | gcagagggcc | tggtgctctt | ccccaggaag | aagaggggct | ttgtgacagg | ctgggggaca |
| 56461 | aaacaagggg | tgctggacac | tgttactgag | gcaactgcct | ggccagtgtg | gaggcagctt |
| 56521 | tagtccctgg | gaccgtttct | gtgcccaagg | agagggaggg | caggggaagg | atggaatttc |
| 56581 | atctagagta | gcaggtcctg | gttgcctggt | aaattatgca | tcaggactgg | gctagccact |
| 56641 | gcctccgttt | tctctgggct | cctgagataa | cctccacctg | cctctagatt | ctgtgccagc |
| 56701 | tgatcgttat | ctatctcatc | aactcttgtt | ccatcaggaa | actgcatctt | ggtgagtggg |
| 56761 | aagtttttgt | gactgtggac | caaacctggg | cttcccatat | tttgggttga | tctgggcctt |
| 56821 | ggagggaaga | gctttcggtt | ttgtagaaat | gatgtggata | ggaactgggg | taggcacagt |
| 56881 | ccatccagtc | tcacacgtga | atgaaccaac | ggggatttga | gaatggcttc | tagatcgtac |
| 56941 | ctgcccctcc | ggccagcccc | ggggctccct | tcccctgcac | ctagcctgaa | gttttccacc |
| 57001 | attaacggca | cacctggggc | agaagacaga | gtagggtcct | tgctccagga | gatcgttgta |
| 57061 | ataatcagat | agagacaaga | cctgagctgg | gaggaagcca | ggacacgggg | gatgggggac |
| 57121 | caggccctga | caaacggagt | gctccgtgtg | ggggctgaaa | gggaaggagg | agagggaagg |
| 57181 | cagggccatt | tagcttcacc | tcaaaggtgg | ggtaaagtga | ggctggtggg | tcctgtcttc |
| 57241 | tctggagatg | ctccccgatg | ggaggaaagg | ccagcttcct | gccggccact | tgcagaagcg |
| 57301 | agcatccagt | gcttccgcca | gcctgtccct | caccccagac | ccagccctgt | ccttcccatg |
| 57361 | tctgatgctc | ccacacgggc | ccccttatta | ccccgtgcca | ttgcagcagc | ctcccggctg |
| 57421 | gtttctttcc | cctgcaatgt | ggctgcccaa | gttctcctcc | caaaacccac | atctcctggc |
| 57481 | ctgccaatcc | caggctggaa | actgttccat | ggcctccaga | gccgctcaca | aacccttccc |
| 57541 | gccccactcc | ctgcctgcac | cttaggctca | ttctctgccc | tccctggcca | cacacctccc |
| 57601 | tgactttccc | caaacatgca | ggtctttcca | tgtctctgtg | cgtttccatt | tccctctgca |
| 57661 | tggaacactc | tttcccatca | cacctaccct | tgcccatctg | ccaaaccctc | caggattctg |
| 57721 | ggtcaacatc | atccccacgg | tggatccatt | ggtgagagca | ataatgaagc | aggtttgcca |
| 57781 | ccaggctccc | aaagaagtgg | gaacactgac | tgcaaagggc | acaatctgat | tggatgaaaa |
| 57841 | gcgaggcctc | agaagcattg | gcaYgtgtcc | gcgaagagcc | tagctggagg | tctgtcctag |
| 57901 | aggaggggct | gaaggagggg | cgagaatggc | agcgctccat | ttcatccgac | tgctctgtgt |
| 57961 | tcctcacatc | tgacccaacc | ttccgttaca | acacaggcac | atttgtggat | actcttggtt |
| 58021 | acatgtgtgt | tctctcctta | gattctgagt | ctcttctagg | aaaggagtgt | cttaatcatc |
| 58081 | tttgtattcc | cagaagctag | tttagaggct | agtatgtagt | aggttcgcag | aaaatctttg |
| 58141 | aatagcggct | gaggtgggaa | gatttcttga | gactaggaat | tcaagaccag | cctgggtaaa |
| 58201 | ctagcaagac | ccccatctct | acaaaaaata | aaaaattagc | caggtgtagt | ggtggatgca |
| 58261 | tcccagctcc | tcaggaggct | gaggtgggag | gatcacttga | gcccaggcat | tcaaagctgc |
| 58321 | aatgagctgt | catcccgcca | gtgcactcca | gcctgggcaa | cagagtgaga | ccctgtctca |
| 58381 | aaaagagaaa | gaaaatcttt | gagtaaataa | atatatgttg | aacttggaag | tattccttta |
| 58441 | cctagaatat | tgttctctgc | aaattttgta | ctatcacttt | ctccaaaaca | taactcagga |
| 58501 | ctcgcctcct | ggaaaaagta | ggttcttttt | tttttttttc | tttttgagac | agagtcttgc |
| 58561 | tctgttgccc | aggctggagc | gcagtggcgc | cgtctcggct | cactgcaagc | tctgcctccc |
| 58621 | gggttcacgc | cattctcctg | cctcagcctc | cccagcagct | ggggctacag | gtgcacgctg |
| 58681 | ccacgcccgg | ctaatttttg | tatttttagt | agagacaggg | tttcaccgtg | ttagccagga |
| 58741 | tggtctcgat | ctcctgacct | tgtgatccac | ctgtctcggc | ctcccaaagt | gctgggatta |
| 58801 | aaggtgtgag | ccaccgcacc | tggccaaaaa | gtaggttctg | attcagtagt | tgactgtgca |
| 58861 | tcagaatgtt | actaagcacc | tattaggtac | taggcatttt | ctaggcactg | gaggcctgtg |
| 58921 | agatataaaa | gagataaagg | ttcccgtgcc | caagttgtta | atgagtgccc | ccaacccatg |
| 58981 | caggccactt | ccttatctgg | caaaatcatt | tgggacgtca | gtaaacactc | aatgttcctg |
| 59041 | gtatccatag | tgccagggac | atggtcagca | gtcagatgtg | actcactgtg | ggcccagcat |
| 59101 | cataagaggc | aggcatagaa | ggcctggctt | tgcattcata | tagctcacaa | cttcactgag |
| 59161 | gagaccagtt | gtttatggga | ttgcaggaaa | ccccaggaag | ccccagccag | ggaagagctt |
| 59221 | atgttgtcaa | attcacagcc | tgtaggaatt | cactgggtcc | actagttctc | ttgcacaggt |
| 59281 | gctactttcc | agttatttcc | tggttttata | aatatgtgtc | tcctctttca | actccttgac |
| 59341 | aagctcttta | cggaaaggga | catcagggcg | tcatgtctag | cgtctagtag | cttctttccc |
| 59401 | tgatcctcat | ctcYgcacct | ggtacagggc | agtgcatgaa | tggatgacaa | atgatcacca |
| 59461 | ccattaacag | gcatttgtca | gccgggcacg | ggggctcaca | cctgtaatcc | cagcactttg |
| 59521 | ggaggccaag | gcaggtggat | cacctgaggt | caggagttcg | agaccagcct | gaccaacatg |
| 59581 | gtgaaaccct | gcctctacta | aaaatacaca | aattagccgg | gcacaataca | ccgataatcc |
| 59641 | cagctactca | ggaggtggag | gcaggagaat | cactggaacc | cgggaggcag | aggttgcagt |
| 59701 | gagctgagat | tgagccactg | cactccagcc | tgggtgacag | agcaagactc | ttgtctcaaa |
| 59761 | aaaataaaac | aaaaaaacaa | aacaaaaaaa | acgaggcatc | catcactggg | tctcttcatg |
| 59821 | agacacaacc | aagaaacaca | atgtgagtga | attctccagt | tctggtagat | gaaggagagg |
| 59881 | gaaggagggc | ttctctgctg | acttagtgcc | tctcttttgc | tcaatgttac | catcagattc |
| 59941 | tgaatgtttt | ctacgagaaa | ttactgagat | gcaaatcaag | aagtgtgaat | ttaacaagag |
| 60001 | ccagttggtt | tcctactttc | atttgggtgc | ctttttttgt | tttttgtttt | aaacagggtc |
| 60061 | tcactctgtc | acaYaggctg | gagtgcggtg | gcaccaatca | tggctcactg | cagcgtcagc |
| 60121 | ctcccggcag | catcaacggc | accgggcctg | ggtgcctctt | aactggttat | ctgtacctgt |
| 60181 | tactgctcgg | tggtctaagt | gggtgagatc | aatttcaggg | tttctccagt | tcaatgtgag |
| 60241 | cttgtggtga | gagaagagtg | tccctggtcc | ctcctgctgc | tttttgctct | ggttcccagc |
| 60301 | ttgctccttg | caacttcttc | ctagtccagg | acagatttac | tggaagtttc | tttagaggca |
| 60361 | gtgctttagg | gctttggtct | tcatctgttg | ggttgtactg | tttgtcgttt | gtcatgtttg |
| 60421 | ctttgcaagc | ctggtggcta | atgttgcttg | cattggctca | ctgctcttgg | ggccatctgt |
| 60481 | aacctgtatc | aaccccggat | gctttgaagt | tcagtgtgca | ataaaaagtt | cattaaaaag |
| 60541 | atgtggaatc | atgaaaataa | ggttacagaa | atgttaacaa | aggatcatat | cctgcttctg |
| 60601 | gtacttttct | tttttaaaaa | atgtatttct | ttgagaaatt | tatgtcagca | tttaaaccat |
| 60661 | ggaagagttt | aggaaggaca | ggcctgggtc | aaaaggaggg | tgagattctt | gaggagatat |
| 60721 | cttggcagag | ccccagcctg | ttgtcttaat | tgagagctca | gggtctaagg | agagaatgtg |
| 60781 | agccagaaaa | aaaaaaatct | aggtttctta | tgactaacag | tttgaaccca | gctttttttt |
| 60841 | tttttttttt | ttttttttga | gacggagtct | cattctgtca | cccaggctgg | agtgcagtgg |
| 60901 | cacaatctca | gctcattgca | acctctgtct | tctgggttcc | agcgattctc | ctgcctcaac |
| 60961 | ctcccaagta | gctgggatta | caggcgtgtg | ccaccacacc | cagctaattt | ttttgtaatt |
| 61021 | ttagtaaaga | cagggtttca | ccatgttggc | caggttggcc | tcaaattcct | gagctcaagt |
| 61081 | gatcctctag | cctcggcctc | ccaaagcgct | gggattacag | gcatgagcca | tcacacccag |
| 61141 | ccaggttcct | ctttttcgca | ggtcctccat | gttcgggtgc | ccacacgtaa | tacacaacca |
| 61201 | gcccctgcat | ccttgctctc | ctctattttt | ggggggcaaa | tatcctagag | gctcattcgg |
| 61261 | ttatatgacc | attgatgacc | atttaattac | tactcatgtt | taataataag | caattatgtg |
| 61321 | atcgtcactt | acttttcagc | cagagggtca | ccatgtgtac | acttagcagc | tgctaaatct |
| 61381 | tagtcctaac | aatgatagtc | ccatatcctc | ccaagcgacc | gtgaggacct | caacagcttg |
| 61441 | cacatctaat | aaacagcact | gcagggtttt | cagggctgcc | accggacagc | tctgggtcac |
| 61501 | agccagactt | accctctctc | tttccacctg | gttgtggttg | agcctcatga | gttctgcaaa |
| 61561 | cctctgctca | tagaggtgaa | gcagcagcac | caggtacaag | cctgaattca | tcagctcgtt |
| 61621 | ttgtgcctac | aaatggaaaa | gaaagctctt | cagttcatct | gtaaaatagt | catggtctaa |
| 61681 | attgtgggat | gcagggtaac | aaggctaaga | ggtgacaaga | actgggagaa | gagcccaagc |
| 61741 | tccaaggcaa | ggcctgctct | tggggaccag | aagacagagc | aggaggagat | ccagggtaga |
| 61801 | ggccagggca | gccactcccc | tgggattttt | tactagctag | tggaggatgc | tcggagaaag |
| 61861 | ttccattttc | caggctcaat | cccattccac | atttattgag | cagttacata | tgcaaagcct |
| 61921 | tgagctggat | gttgtgggga | acacagggca | aaaagcctca | gaactccctg | ctgggaaccg |
| 61981 | atcacctggt | gtgggggaga | gagcttcatc | ggtaaacagc | tccagtacaa | gctggatgtg |
| 62041 | tcgggtgcta | caacagagat | gcaccaggcc | ttgtccataa | cgttctcagc | agtcacagat |
| 62101 | gtccccttgt | gaaaggcttc | ctgaggagcc | tcctttgccc | ccatcattcc | tttgcttccc |
| 62161 | agtccctaag | cattcgtctt | gatcttgcat | atcacctttt | gcacttgtta | acgtttccgc |
| 62221 | tttctcttga | ggagtgggct | gtgctttttt | tctcgctggt | gctcagaaca | ggcgctacac |
| 62281 | agggaggatg | tgatggtctg | gctgcccatg | gcacttctct | gtccttagca | cttgggcttt |
| 62341 | gctgccaaga | gtggctgtgc | tcactctaga | cctttcacag | accttccata | aatcagtggt |
| 62401 | attagtaaca | actactccta | tccctctata | ggggaaggag | gggaaggccc | tttcagtccc |
| 62461 | catctttccc | ttgagtgggg | gtcttgggat | gggccaggct | gtgctcacaa | agccacccag |
| 62521 | cccccgggga | atagagtccc | cagcagaggg | catggcccct | tccgtcaccc | ttgggaaagc |
| 62581 | ttgcaactgt | gactctgtcg | gcccaggata | acgatgcgtg | ggcatgaatg | acacagtgag |
| 62641 | cactcagtgt | ctcatctgcc | ttcttcctgc | agcagcgcgg | gtgccacatc | ttcctgttct |
| 62701 | cattagatcc | agggcagctc | aggaggttgg | cacagaaccc | acctctccca | ggaccaagca |
| 62761 | gctgccagaa | aaggcacaga | agttgagcaa | ggattgatat | gctcttcctt | ctgaatcagg |
| 62821 | gccaactctg | gatcttggtg | acagccggcc | ctgagatcac | tgctgcaggc | ctctccacca |
| 62881 | ttctacagat | ccatcttcga | aggccccagt | cctctgtcct | tagcagttag | cctcctattt |
| 62941 | ttagcctttg | gtcgtgtttt | ttgtgctctg | ctgaccttgg | cctagagtga | gtagagaaga |
| 63001 | agttgcttgt | ttgcattatt | tctgttttat | ttatttaatt | ttatttttga | gacagacaca |
| 63061 | gtcttgccca | ggctggagtg | caatggcgtg | atctcggctc | actgcaacct | ccacctccca |
| 63121 | ggttcaagca | attctcctgc | ctcagcctcc | tgagtagctg | ggattacagg | tgtgcaccac |
| 63181 | cacgcccagc | taatttttgt | atttttagta | gagatggggt | ttcatcatgt | tggccaggct |
| 63241 | ggtctcgaac | tcctggcctc | aggtgatcca | cctgccttgg | cctcccaaag | tgctgggatt |
| 63301 | acaggtgtaa | gccaccctgc | ctagccactt | tttctgtttt | aattgcctcc | tcctgccctc |
| 63361 | cccgtgagtc | tgtgcctgcc | tccgactgtc | tcgccaacca | cctcttctga | gaagccttcc |
| 63421 | tcacctggct | tttagctcct | cctcaccaac | aaagccttgg | tccttatttg | tttacctcat |
| 63481 | ccccggcttc | ctcctgcaaa | agctgccagt | ggttctctca | ccagtgctct | ttgccagcca |
| 63541 | gcttttatgc | ttgagacacc | gggatgcatg | atgacaaatc | aatcagctag | aaaatggttt |
| 63601 | ggaactatca | ggtttgtcaa | agcattcagg | aaaatcattt | actttttatg | actcaataag |
| 63661 | gcccagaggc | atgcagatgg | gagaatatcc | actaatgttt | tctgcagata | atttgaaaag |
| 63721 | tcacttctag | cagattgtgt | ctcttctctg | atctgtggga | gggaacctgc | cacacagtgc |
| 63781 | catcaatctc | gccggctcgg | ttcctttatg | tgcgcccttt | gcagagctgg | tgatgctcga |
| 63841 | caaaggggct | cagcccacca | atcagagcag | gcgtgtgata | gtcaactccg | aatcagcctt |
| 63901 | tacactcctg | agctacaagt | gggagggatt | tgttaactca | aagagttcca | ccgcacttgg |
| 63961 | ctggccaggg | ccaaatgaac | gaggttttgg | agcagggctt | actggagcta | cggcccatcc |
| 64021 | accccttcct | tgtcccaatc | acatcacact | ttgactccca | cgtggagttc | cctttgtgtg |
| 64081 | ccagactgaa | gggaatgggg | gtggcattaa | taggaggggg | cggggacatt | aaaaaggaga |
| 64141 | aggcatgtga | aacagcgcaa | aagggctccg | gagatctcag | cccacctgcc | agctgttttc |
| 64201 | tccattttgc | taatattcat | ttaggccaag | agacttgcta | ctgacggaaa | ttctcaatat |
| 64261 | attccactta | aagaagctga | cagcctggct | gaatgttcta | tcaagtcttc | aaatatattc |
| 64321 | aagagtgctc | ctaggtataa | gggtgattag | gtgactgcgt | gtgtctgagg | acatctgtcc |
| 64381 | ttggctgaga | tggagccagg | ccaggcctgt | gcttcacagc | aatgacagcc | tgaggcccac |
| 64441 | tgagactctc | ctgtgcggca | gagcaaggat | ggcaggggtg | tgttccaatt | cagtgagcag |
| 64501 | gggcccctgg | agtgcatctt | acctggaaag | cgctgccggc | taaggtgggc | agtgcagggc |
| 64561 | caccggacac | tgtgacttta | ggcagagttt | taactgtgca | ggaccaaagg | gactcaattt |
| 64621 | gtaatggaag | ggaagtaacc | tctgttctgc | ctgtttcctc | aaagcatgat | agtaaagtga |
| 64681 | gctaatatat | gggatctcgc | aacaccaaca | catcctacaa | gagacaagat | ttaaagacag |
| 64741 | gattccaggt | aaattgtctg | ggcgttaaga | tctgtgaggg | caagacctgc | atctgtcttg |
| 64801 | tttgctcctg | actcctagtg | cctggcccag | tgcctggtat | actgtgcatg | ctttataaat |
| 64861 | atatgcacag | atgaagcaat | gagaaaaaaa | tgcataattg | tgatggatca | gacagtgacc |
| 64921 | cattcagcca | caacttttga | atctagtggc | tccaggttta | acatgcatca | gaaatactga |
| 64981 | gcaggccttt | tcaaacacag | ttgccaggca | gtcccgtccc | ctcccctccc | ccttctcctc |
| 65041 | tttttttgag | acagtctcac | tctgttgccc | aggctggagt | gcaatggtgt | gatctcggct |
| 65101 | cactgcaacc | tccgcctccc | aggtttaagt | gattctcctg | cctcagcctc | ccgagtagct |
| 65161 | gggatttcag | gtgtgtgcca | ccactcccag | ctaatttttg | tatttttagt | agagacgggg |
| 65221 | tttcaccaca | tttggccagg | ctggtctcca | actcctgacc | tcaaatgatc | cacccacctc |
| 65281 | ggcctttcaa | agtgttgcga | ttacaggcgt | gagtcaccgc | gcctggcctc | actgctcctc |
| 65341 | ctttcctccc | tgcatttctg | attcagctgg | actggagtgg | gcccaagaat | gtgtacttct |
| 65401 | aacaagttcc | cagatgatgc | taacgctcct | ggtttgggga | ccacccattt | ttgagaacca |
| 65461 | ctgatctaaa | ctaagggtga | gcacgtggta | gctgctaagc | tgaatctggc | ctgtggatgt |
| 65521 | gctctggttg | gatggaagga | agcaagaagg | aaggtagaaa | agaagagcat | gaccatcttt |
| 65581 | agttggggag | agcactttcc | attttgccac | aaatgccacc | acgcccaatg | gcaggatacc |
| 65641 | tagaagatta | ccatcatttg | ttgtctgcct | ggcctctgtg | gtatttgagt | gtgtaacctg |
| 65701 | tgacctaagc | tttccttaaa | Yctgttttca | cctcatccat | tttggaggct | agaatgttat |
| 65761 | ttggctcgcc | gcatgctgct | gtccaggagt | tctgccctcc | accgcaaaac | cgagtgccca |
| 65821 | accagcatcc | aatatgagtc | ctgcttccag | gcgacagcca | acgctcacag | gagcaaatgt |
| 65881 | aggcttgcct | cacacacagc | actaccgaaa | gagtcaggag | aaaggagggc | tgtactgcca |
| 65941 | caggccctga | cagacctaag | atgaaaccgc | caggtccctg | gggtcccaca | gctgctcttg |
| 66001 | tctaatgatc | ccctctccag | atgcttgtag | gagttcctga | tctagccgct | tgaggcctgg |
| 66061 | taacagacct | tgaccttggg | cctcatacta | gaataatccg | agttcagtgc | cctgttagca |
| 66121 | cctgtgagca | gcatctgccc | tgctcccacc | agaagtacag | aactaaggta | ggaaaacgtg |
| 66181 | ctctgacggg | agcccctcca | cactctcaaa | ggccaccgac | agtgctgacc | cggcacctag |
| 66241 | aggacacgcc | ctgatctgtg | ctcaggtccc | aggggaggcg | tccgtcccag | atgtcacaga |
| 66301 | cttcactcca | aggcccggtt | gatagaaggc | ccgctcctct | ctttccttcc | gctgttaaac |
| 66361 | atctcttctt | cagaaaggcc | ttctctggtt | cagagagcaa | gtcggagtcc | cacagctgtt |
| 66421 | cccgcagcgg | cctgcatggt | ccttgttttg | acctccacct | gcaggaattg | ctggcttcag |
| 66481 | tcttcgctgc | tggtcggtag | ggccctgcgg | gtcttgggta | cggcagcatt | ttcggcacag |
| 66541 | tatctcacag | gcagcaggtg | ctcaataagt | atttgaaaat | ttagttaaac | tggctgggtg |
| 66601 | tggtagctca | cacctgtaac | cctagcactt | tgggaggcca | aggtgggaag | atcacttgag |
| 66661 | cccaagattt | tgagaccagc | ctgggcaaca | aagtgagaca | gaccccatct | ctcaaaaaaa |
| 66721 | aaaaaaaaga | aaaaaagcag | ggtgtggtgg | cacacacctg | tggacccagc | tacttaggag |
| 66781 | gctgaggcag | gaggatccct | tgaacccagg | agggaccctg | gccccatccc | agagctgggg |
| 66841 | ttagacctgg | gttgggcctg | tgtatcacca | ttttaataga | aaaaaagaag | acctgcagct |
| 66901 | aaatgttata | agttatcatt | ttgggtggta | aattataaga | aatttttgta | tggtttttgt |
| 66961 | aattaaacaa | aagcaaaaaa | aaaaaaattc | taagtgggaa | aatatataag | aaaaacagtt |
| 67021 | cactagtcag | ttagtttgag | gttaagtgag | aaccaggcta | agctgaagcc | cgttctaggt |
| 67081 | ttcttttggt | tttgcccctt | ttttggtaca | tgtctagact | tgtacacgtc | tagaagtggt |
| 67141 | gccacttctc | cagaaaggaa | cccatttgcc | aaggacaagc | agcaggaggg | tggccaggag |
| 67201 | gcccaggtga | gccaggtttg | gaagcgaggg | gaagggacca | ggcccacaag | gcacaggtgt |
| 67261 | ccatgtcacc | tccacggcca | ggctggtggc | ctcctcagct | gggcttgtcc | cctcagtggg |
| 67321 | ctcaccgcgc | ctcggggacg | agctgagttt | cagtgacgtt | gtagatatct | ttgtgttcaa |
| 67381 | caacagttgc | ttcagtcttt | atctatggcg | agcttcagaa | ttcgcccaag | tcaggctccc |
| 67441 | tggggcctgg | ggtttcgccc | aggctgtgag | cagacaggca | ctatgaattc | ccagcaggtc |
| 67501 | atggggacag | tcagggctgc | agtgaggtgt | gagccctcag | ccgcctccag | acagcagccg |
| 67561 | cccatgctat | gacctggctc | tccccacagg | gcgggcctgg | cagcctagct | gtcacctggg |
| 67621 | atgataatgc | gggcatttcc | caaaggctct | gatggcttgt | gaagttctca | ctgaaacgaa |
| 67681 | tttaaaatca | ttcctgcttc | cacgtggagc | aatgactgtg | ctggatgctt | tctgtctctc |
| 61741 | ctgcagatcc | gcacccctcc | tgctctgtgg | cccaggggcc | ggccccatgg | ctttccccag |
| 67801 | ccgggcttcc | ttgccctctg | gcttcccgtt | gggtttggcc | catgggaggc | cccggcagga |
| 67861 | gacccgaggg | cggaggaggg | agaagctggg | tgtctcttcc | caatccctct | ctgctgggcg |
| 67921 | tggtgtgaca | gtggttgctg | tcttctcctg | aaggccacag | gctgctggtg | gctgatcctt |
| 67981 | agctacagcg | ctcaKgaccc | tggccacact | gctagtccca | ggacgcctcc | tcgttccttg |
| 68041 | ctgctttctt | ttgctgccca | gatttccctc | ctcccttctg | cgttcttcac | ttgccccctt |
| 68101 | tgagtgtgcc | accatctctt | cccacccaga | tcctggctga | tacaattact | gtcatagttc |
| 68161 | tttctagaag | gtgcttgtag | ccagttgtct | taacatggag | | |

Following is a CDK10 genomic nucleotide sequence (SEQ ID NO: 4).

| >16:89456451-89555700 | |
|---|---|
| 1 | gttaggaagg gtgcagtatt ctaaagtgta ctgcagacgt gtgtaaacgc catgcctgac |
| 61 | acacactcaa gcccccacgc cagccggagc aggacggggc agcagcaggg cggctcgctt |
| 121 | gggggcggct gcagctgggg cctctgagtc gagatcaggg ctccctggtc ggagcctcag |
| 181 | cccaagctct cctccagcct ctaacccaca accctctctc ccttcccaca gcaYcagagc |
| 241 | gtggggttgg tgacacagcg tttcggggac cgaccctacc tccgtgacag ctggtcctcc |
| 301 | tggctgcgca cagagctctc gcccacggca gaggcgccct cgggcagctg gctgagctgg |
| 361 | tccagcacct ccaggccatt ctcctcggcg atctgcatga tgaggctgtc cacctgctcc |
| 421 | tgcggcgtgg tcagggtggt ggccgagctc atggagtcct ccatcacctg ggggcagggg |
| 481 | catgctctgg acaacaggtg gggccaggcc catggggcYc cactcagctt cacaagggta |
| 541 | cgactgcact tttcgttccc tcacacagcc agccaccttc ttgccctgtt ctctcagtga |
| 601 | ctctctctgt ctcaccttcc accaggagcc tttcctgccc ccccacccag ccccaccctc |
| 661 | tgactgtgcc atgcctgctg ccgcgggtgg acatctttcc acacctctgc atgcttgtct |
| 721 | gccatccgag cctccccaca aggagccagg tgcctgcagg aggcccccgc cgccctgccc |
| 781 | accctacctg tcagggtcct ggcagggacc cagcacagag gatgcttggt gacgtcaggg |
| 841 | agcaaccaca ggtatgcaga gacagcacgt cagcctgtga gccacccctc ccgatgagct |
| 901 | gcccacacac gctccttgtc cccaggccac agccccaagg gtaggggcca cataccgatg |
| 961 | tatggacgtc caggttctgc acctgctgct cgaacctgtc catcactgag gagaccttct |
| 1021 | gcaggtccat ggtgctcagg gccttgtcca gggctttggt cacctgggcc atattcttgg |
| 1081 | tcacctgaga caggagagag cgcaggaggg aacaggatga aaggcaagtg gagctcacgc |
| 1141 | accagggacg ggtcccctgg actctgacag gagagctggg gcaccccaac cctgacccac |
| 1201 | aaaaccccaa ctctgggctg agccatcatc tgggtcctat ggaaagacca ccaactcgac |
| 1261 | ggaaaaggcc atggagaccc agtgcggggt cagtggagaa aaggccgccg acgtggagac |
| 1321 | ccagtgcggg gtcagtggag aaaaggccgc cgacgtggag acccagtgcg gggtcggtgg |
| 1381 | agaaaaggcc gccgacgtgg ggacccagcg cggggtcggt ggagaaaagg ccgccgacgt |
| 1441 | ggggacccag cgcggggtcg gtggagaaaa ggccgccgac gtggggaccc agcgcggggt |
| 1501 | cggtggagaa aaggccgccg acgtggagac ccagcgcggg gtcggtggag aaaaggccgc |
| 1561 | cgacgtgggg acccagcgcg gggtcggtgg agaaaaggcc gccgacgtgg agacccagcg |
| 1621 | cggggtccgt ggagaaaagg ccgccgacgt ggacacccag cgcggggtcg gtggagaaaa |
| 1681 | ggccgccgac gtggggaccc agcgcggggt cggtggagaa aaggccgccg acgtggggac |
| 1741 | ccagcgcggg gtcggtggag aaaaggccgc cgacgtgggg acccagcgcg gggtcggtgg |
| 1801 | agaaaaggcc gccgacgtgg ggacccagcg cggggtcggt ggagaaaagg ccgccgacgt |
| 1861 | ggggacccag cgcggggtcg gtggagaaaa ggccgccgac gtggagaccc agcgcggggt |
| 1921 | cggtggagaa aaggccgccg acgtggagac ccagcgcggg gtcggtggag aaaaggccgc |
| 1981 | cgacgtggag acccagcgcg gggtcggtgg agaaaaggcc gccgacgtgg agacccagcg |
| 2041 | cggggtcggt ggagaaaagg ccgccgacgt ggagacccag cgcggggtcg gtggagaaaa |
| 2101 | ggccgccgac gtggagaccc agcgcggggt cggtggagaa aaggccgccg acgtggggac |
| 2161 | ccagcgcggg gtcggtggag aaaaggccgc cgacgtgggg tctccaggac tgctgtgccc |
| 2221 | tcctggccca cctgcactgt gtgaaggaaa tgcctgcaac agccaaggct ctgctgtgac |
| 2281 | gtccctgacc cagcagatga gcctggccat gcaggctctg cctcttgtgg gagttgccaa |
| 2341 | gaccgaggcc tggccaggta cagtgacagt gagtggttca cgcctataat tccagcactt |
| 2401 | tgggaggccg aggtgggaag attgcttgag gccaggagtt tgagaccaga gcggccaaca |
| 2461 | tagacacect gtctctacat aaaaatgaaa aaaatcagct gggcatggtg gtggatgcct |
| 2521 | gtggtctcag ctacttggga ggctgaggca ggaggatcgc ttgagcccag gaggttgaga |
| 2581 | ccagagtggc caacatagac accctgtctc tacttaaaaa tgaaaagatc agccgggcat |
| 2641 | ggtggtggat gcctgtggtc tcagctactt gcgaggctga ggcaggagga ttgcttgagc |
| 2701 | ccaggaggtt gaggctgcag tgagctgtga ctgcaccact gcactccagc ctgggtgaca |
| 2761 | gagcaagaca gtctctaaaa aatatatatt taaaaaaaaa aaagcccaag tcttatcaag |
| 2821 | aggtccttcc tggggcatca gtgcaagctc agggtgcagc ctcacctgtg tccctgccag |
| 2881 | catttcccac cccacgctga tccagcttaa ctcaacctcc ccaccccacg ctgatccagc |
| 2941 | cctcaacctc cccaccccac gctgatccag cttccctcaa cctccccacc ccRcgctgat |
| 3001 | ccaccttccg tcaactctga gctgccccag atccagaccg cagagcccat tcctgcttca |
| 3061 | gccagcgaac gccacccatc cagcaccagg gcccagcact cacccccttc atagtcacag |
| 3121 | ctgtctgcac cttggaggcc actgcgtcta cgcgggacgc catccgaagc cagttcacac |
| 3181 | cttcgttctt cttgcggatg gcgttctcgg catacacacg ggcacactct acatttttct |
| 3241 | gcagaagggc ctgaaacccg Sgggggaaag cagctggaag agcttggtgg tgtgtgtgcc |
| 3301 | ccctgctagg agcccagttt ttgttttgtt ttgttttgtt ttgtttgaga cggagtcttg |
| 3361 | ctctgttgcc caggctggag tgcagtggca cgatctcgac tcaacgcaag ctccgccccc |
| 3421 | caggttcatg ccattctcct gcctcagcct cccgagtagt tgggactaca ggcgcccgcc |
| 3481 | accatgcctg gctaattttt ttgtattttt agtagagatg gggtttcacc gtgttagcca |
| 3541 | ggatggcctc gatctcctga cctcgtgatc tgcccgcctc ggcctcccaa agagcYggga |
| 3601 | tcacaggcgt gagccaccgM gcccggccct aggagcccag tgttgaggcg gtgggcgggc |
| 3661 | ctctccccag aaatgtttga gctaaaaatg caaacaccca ggccccagcc cagcccccca |
| 3721 | agcaggcttc tgcttccccc cgcacccctc cagcactcca ctgggcacgg gacacagcgg |
| 3781 | gtgacactca gagcctgtgg caggcagttc tccgagctcc attctccctg ggagcctggc |
| 3841 | tgtgggtgaa gagttaccaa agtccaatac gctgggacct acttcaccga ttacgcagtt |
| 3901 | ttaagagctg cacaagatcc taccttcttt tctttttttt ctttttttgg agatggaatc |
| 3961 | tcgctctgtg gcccaggatg gagtgcagca gtgtgatctc ggctcactgc aacctccacc |
| 4021 | tcctgggctc aagcaattct cccgcctcag ccttccgagt agctgggatt ataggtgtga |
| 4081 | atcactgcgc ccggcctaga tccctcctct ccttctgccc tgtccatcat ggggagggga |
| 4141 | cccctcctgg agaggcaccc ctgtaagcct ccagccccat cccaccccat ggggcagggg |
| 4201 | catcatgact ctgcccagac cctgatgccc tagacaccag ctctgtaggg aggagagags |
| 4261 | taaaaatgat aggatccgct gggcccagtg gctcatgcct gtaatcccag cactctggga |
| 4321 | ggctgaggca ggtggatcac ctgaggtcag gagtttgaca ccagcctggc caacacggcg |
| 4381 | aaaccccatc tctactaaaa atacaaaaat tagccaggtg tggtggcgca tgcttgtaat |
| 4441 | cccagctacc caggaggttg aggcaggaga atcactcgaa cccaggaggc agaggttgca |
| 4501 | gtgagccgag accgcacctc tgcactccag cctgggcaac agcgagactc catctcaaaa |
| 4561 | aaaaaaaaga taggatcgtg gcagacagct agctagcagc cctcctcctg cagatggcaa |
| 4621 | aatcaagttc agaatgttgg aaatgcaaag aaggaaaaca atgaaggcaa tgcactcatc |
| 4681 | tctgtgctct ggacgccctc agtgggctgg gaaggtctgg caggggctgt agggcaggga |
| 4741 | cRgaaaccat tctgggctgc accacacaca gcacctccct tcctgagtgc ctgtttgtct |
| 4801 | tatgtgggaa gcaaatactt tgaggctcag aaatcatagt gaggattagg ctgggcacag |
| 4861 | tggctcatgc ctgcaatacc agcacttttg gaggccaagg caggtgggtc acttgaggat |
| 4921 | aagagttcga gaccggcctg gccaacatgg cgaaacccca tctctacaaa aaaattaaaa |
| 4981 | aattagctgg gtgcaagcct gtaatcgcag ctacgtgtga ggcgaaggcc ctagaatcac |
| 5041 | ttgaacccgg gaggcggagg ttgcagtgag acgagatcgc acctgtgcac tccagcctgg |
| 5101 | gcgacagagc aagactccgt ctcaaaaaac aaacaaacaa acaaacaaac aaacaaaata |
| 5161 | tatatatata tgtacctcaa ggtgacgatt aaatcatgat gtagaaagtg ccctgcccct |
| 5221 | ccccaaaagg catcaaaaca aaacaggaca caaaaataag ggcagaaaag gacagaagac |
| 5281 | aaaccaggag agtcatgacc ccacagcccc cagggtctca ccttcttcac tttggcctgc |
| 5341 | tccgccttgg agtccttctc cgccttcttg gccagcttct ccagctgctt cgccgtgaac |
| 5401 | tgagcggaag ccggaatgtc ctgggtcaga catgcggagc ccatccccca ggcccggctc |
| 5461 | tccacacccc cacacctgtg cccacacctg tgcccacaca ggttcctaag cccccatcag |
| 5521 | ccagcctggg cccagcacac cgcagggaag gcgtgaagcc tgtgggcgtg catcctgggc |
| 5581 | ggcgagagat ggcaccagct ggaggaggaa gctgcctgct ctgcctcagg ctctgtctgg |
| 5641 | gttttctcca gtgtctgtct tctctgtgaa cagcctcacc caaggcctcc tccagagcgg |
| 5701 | aagggtcaaa aacctctcac tctgatctcg gctcctgctt atggatggct tttcaacgtc |
| 5761 | aaagaaagac tgaaggaaac tcatttcagg ctacccccaa gtcataacac agaactccag |
| 5821 | gtaaaaacac acctgaaagt gttatctcga gagaatgctg tggatgcagg aggttgctgg |
| 5881 | gcagcacaag cagcttccgg ggatctaact gctctgccag caccacacgg aaacagggcc |
| 5941 | tctggcaaca ccacaaggac acaggacctc tggcaaccca cccggaacca cagacgccct |
| 6001 | ggtaccaggt agccctggcc accagcacct caaggaaaca gggcctctgg caacccatct |
| 6061 | ggaatcacag acaccctggt atcaggtagc cctggccacc agcacctcaa ggaaacaggg |
| 6121 | cctctggcaa cccatctgga accacagacg ccctggtacc aggtagccct ggccaccagc |
| 6181 | acctcgagga aacagggcct ctggcaaccc atctggaacc acagacgccc tggtaccagg |
| 6241 | tggccctggc caccagcacc tcgaggaaac agggcctctg gcaacccatc tggaaccaca |
| 6301 | gacgccctgg taccaggtgg ccctggccac cagcacctcg aggaaacagg gcctctggca |
| 6361 | acccatctgg aaccacagac gccctggtac caggtagccc tggccaccac agcaccacag |
| 6421 | ggtgcaggac acaagcaggc ctcagccggt cccctctgtg cccaacagca actgacaggc |
| 6481 | ccaccgtgat gggaaaacat cccaaagctg catacggtag ctccggcaca tcactcctgc |
| 6541 | ccagtagctc agagccagcg acaaggcaag ataaagctgg ccacagatga gcagccagga |
| 6601 | gcacatccca gggaaatgca cggaaacctg ccctggatta ccggaagctg caacctccac |
| 6661 | cccacgtggg aggaatactc agaaagctct ccttcaaatg atctcaaaga atgagtccac |
| 6721 | acagcggaac accaaggcct aatgcccaag ttgtcttttt tttttttctt ttcttttttt |
| 6781 | tttttttttt ttgagacgga gtctctgtca cccaggctgg agtgcagtgg cacaatcttg |
| 6841 | gcttactgca agctctgcct cccaggttca cgccattctc cggcctcagc ctcctgagta |
| 6901 | gctgggacta caggtgcctg ccaccgcgcc cggctaattt ttttgtgtgt ttttagtaga |
| 6961 | gatggggttt cactgtggtc tccatctcct gaccttgtga tccaccccac ctcggtctcc |
| 7021 | caaagtgctg ggattacagg cgtgagccac cgcgcctggc cccaagttgt ctttcttaag |
| 7081 | ataacactcc gggccaggca cagtggctca cgcctgtaat cccagcactt taggaggccg |
| 7141 | aggtaggcgg atcacgaggt gaagagatcg agaccatcct ggccaacgtg gtgaaacccc |
| 7201 | gtctctatta aaaatgtaaa aaaaaattag ctgggcatgg tggtgggcgc ctgtagtccc |
| 7261 | agctactcag gaggctaagg caggagaatc gcttgaaccc gggaggcaga ggttgcagtg |
| 7321 | agccgagatc acgccaatgc actccagcct gggcgacgga gtgagacgcc gtctcaaaaa |
| 7381 | aaaaaaaaaa aaaaaaaaag ataacactat ttctttcatg gctttcatgg atgaccctaa |
| 7441 | ggatctgcct ccacttagcc tcagataaga aaagagctaa tatgtcacca aggtggctca |
| 7501 | ataatcccct ccttggtcag cagtctgagc cctgtgtggc ggtcactcaa ctgttatata |
| 7561 | atctgcccga ctgtttctgt ggctctgagt gagcgtggct tatactatct gtcctcacca |
| 7621 | gaacagggct ggggtgaacg gccattcggt acataccttc aactggaaca gggtatctgc |
| 7681 | aaagaaagag ggaattaatg gtttgaggat tgggaatggg acgttacact tctggcaggc |
| 7741 | aggactcact aggttccttc tagacaccag gagctagaac acacacacag accacacctg |
| 7801 | cctggggcct tcggggagcc ccccccaaca ggggaggcac gagacagatt cgtgtgccag |
| 7861 | ccctggctcc ttgctagcca cagggcccag caaatgcaga ccctccctcc ctgtttcctt |
| 7921 | gttgggaaca tgagggtggt accagccaat ctcaaagggt tagtgtgagg acagaagagg |
| 7981 | tcatggacgc agcatctttt ggccatggtt agaatccaaa agatggtggt ttataattaa |
| 8041 | tgcaactttg caaaWgtccc ttgggattct ttcccaattt aaaagttcac ctgtaatccc |
| 8101 | agcactctgg gggtgccgag gccagtggat cacttaaagc cagggtttga ggccagcctg |
| 8161 | ggccaattta aaagtKcagt acttgacagt ttacttttta cagactcttt ctcctcaaag |
| 8221 | gtaaatggat aaaaatagca aggtggtaac tcttagacat caaagagctg atttccagcg |
| 8281 | gtacagtcRc tcacgcctgt aatcacagca ctctgggagg ctgaggcggg cagatcactt |
| 8341 | gaggtcagga gttccagacc agcctaggga atatagtcag acctcatctc tattagaaaa |
| 8401 | atatatattt ttttgccggg tgcggtggct cacgcctgta atcccagcac tttgggaggc |
| 8461 | cgaggccggc ggaacatgag gtcaggagat cgagaccatc ctggctaaca cggtaaaagc |
| 8521 | ccgtctctac caaaaaatac aaaaaattag ccaggcgtgg tggcgggcgc ctgtagtccc |
| 8581 | agctactcag gaggctgagg caggagaatg gcgtgaaccc gggaggcaga gcttgcagtg |
| 8641 | agccgagatt gcgccactgc actccagcct gggcaacagt gcaagactcc gtctcaaaaa |
| 8701 | aaaaaagaaa aaatattttt tttaaaaagt aggattgatc tcaattcatt caactgctaa |
| 8761 | atttggagag cgaccctgac cccagattcc tgaaagggct ttaccacact agccacaacg |
| 8821 | aggaaatgga gcctgtaaaa gaatgaggtt actgagtgtt cacaaatgca caccaaggct |
| 8881 | ccaagatgag atctcccggg gttccagaaa gctctgggca tcgttcaatc tatcgcctgt |
| 8941 | cagtgcaggc gtggtcaagg ccaagttcaa cctgtcatct atcagttcag gtgtggtcac |
| 9001 | ggccaaggtt atctgtgact ggaaaacagc cagcctgacc cagatggcac agtgcaggcc |
| 9061 | aggcgggccg tcccacccaa ctcactgtgg gatgctggcc aaaccgccct cctcatctca |
| 9121 | gctttccctc acctcagctt tccctcacct cagctttccc tcacctcagc tttccctcac |
| 9181 | ctcagctctc ctcacctcag ctttcctcac ctcagctttc cctcacctca gctttcctca |
| 9241 | cctcagcttt ctcttccagc cgtgtgtggc agcacaaaaa cccagctaag gcaaaatctg |
| 9301 | actctgagga aatctgacat gggcttgaga gaagtcccct caaaagagtt gagtgagctg |
| 9361 | tggagtgaaa agccacaatt tttctttttt ttttttgaga cggagtctca ctctatcacc |
| 9421 | caggctagag tgcaacggca ccatcttggc.tcactgcaac ctctgcctcc caggtttaag |
| 9481 | cgattctcct gtctcagcct cccaagtagt tagaattaca cgtgcacgcc accaggccca |
| 9541 | gctaattttt gtatttttag aagagacggg gtttcaccat gttggccagg ctggtctcaa |
| 9601 | actcctgacc tcaggtgatc aaccaacctc ggcctcccaa agtgttgggg ttacaggcgt |
| 9661 | gagccaccac atccggcctc ccaaagtgct ggaattacag ccgtgagcca ccgcgcccgg |
| 9721 | cctcccagag tgctRgggtt acaggcctga gccaccacac ccggcctggc cagcatattt |
| 9781 | tttgtggcgg gaggtagatg ttatctttca tcttctctca gggcagaaaa tctctacaca |
| 9841 | tttccttcat gatgatgaca cggtggtact atagcttttc acacttctaa ctaaaagaat |
| 9901 | tttttttgag acggagtctc gctctgtcgc ccaggctgga gtgaagtggc gcgatctcgg |
| 9961 | ctcactgcaa gctccgcctc ccgggttcac gccattctcc tgcctcagcc tcccgagtag |
| 10021 | ctgggactac aggcacccgc caccacgcct ggctaatatt tttgtatttc tagtagagat |
| 10081 | ggggtttcac tgtgttagcc aggatggtct cgatctcctg accttgcgat ccgccggcct |
| 10141 | cggcctccca aagtgctggg attacaggcg tgagccaccg cgcccagccc ttcaaaaagt |
| 10201 | atttttaaaa gcctgcccat cacaccagcc caacgatgtt tcagcaaaca taggacttca |
| 10261 | ttttgccaga ttcttcctgt tctttggtat ccctgagcac acagccggct agccccagat |
| 10321 | gtagaaggta agaattaggg cgacccattg agcacagaaa cgcttgtgct ggctcctgct |
| 10381 | caaacctctg cttccccaca tggagaatgg acgcaaagag ctgctgtgac cagggcctgg |
| 10441 | cactcaggga gcgctttctg tctgagtcct tctgatttgc agtgatgctg tttggggcag |
| 10501 | ggggtgcaac tgtcactttt tctggtgccc tctgacccga cattccaagt cccgaaaggc |
| 10561 | cgaacaggca tcgcacagtc ccccctaccg agctggcggg aaagggcgcc tgacgctgag |
| 10621 | gagtgccagg ggcgggggaa agggtcctgg gtcggtggtc gggggtccag gccctgggca |
| 10681 | aggggtcctg ggtcgggagt ctaggtcccg ggtccatggt tgggggtcag aggtcgaggc |
| 10741 | cccggggaag gcataccggg ttgagatttg gggatggagg ccccggggaa ggggtcccga |
| 10801 | atcgggggtc ggggatcgag gcctgggggg aggggtcccg ggtcgagggt cgaggcccgg |
| 10861 | ggtaaaggat ctcgggttgg gggtcggggg cctgggaagg gtcccggtcg gtggtcgggg |
| 10921 | gtcgaggccc gggagaaggg gtcccgggtc ggtggtagag gccctgggga aggggttccg |
| 10981 | ggtcggggat cgagggtcga ggcccgggaa aaggggtccc gggttggtgg tctaggccct |
| 11041 | ggggaagggg ttccgggtcg ggtatcgggg gacgaggctc gggaaaaggg gtcccaggtc |
| 11101 | ggtggtcgag gccctgggga tggggtccgg gtcggggatc gggggacgac gaccgggaaa |
| 11161 | aggggtccca ggtcggtggt cgaggccctg gggatggggt cccgggggat cgacggtcga |
| 11221 | ggcccgagct ctcctgagtc ctgcccgcgg ccccaggtgg gcggagccca cgccagtggg |
| 11281 | agaagcggcc ccgccccgcg cgcgagtccc cggaggacgg ccgcgacctc ttaccgtcca |
| 11341 | tggccacaat gacaggagca gcactcggag agggagaagg gacgccaact ccgggcggtg |
| 11401 | tcaggtcccg gcggcgatcg aaccgaccaa gctgcacccg gcggggactt ccggggtcgc |
| 11461 | cgccccactt ccggtcgcac gggatgctct cgcgagagga tggtggccgg tcccgccaga |
| 11521 | ggggcggggc gcggctgcgc ggcgcgcggt cgccatggtg acgcacgccg ctggcgcgag |
| 11581 | gaccttttgt gagtcgctcc cggctccgcg gccgcggagg tgtggggacc cgggcttgcg |
| 11641 | tcggaggggg cggtgggctc acccatgggc ctttccaaaa gcaaagagaa acccaggaaa |
| 11701 | ggtaaaggag gcgcaggcgc tgggctcccc gcgtctccgc ccctgggcgc gggcccaggg |
| 11761 | cggggctggg ctgggcgggg cggtgtgagc gcaggcgcca ggctcggccc ggtgcgaacc |
| 11821 | gttcctgccg ccgagtccgc cacggacggc gcgtttcccg cctgaggcgg ttacggaaac |
| 11881 | gcggagactc gagacttgaa gccagcttat tctggatcca cgcaggcgac tgaagacgat |
| 11941 | gggacccact gccctgcatt cggtaagtcc cgggtctaac ggatgatcca tatatttcag |
| 12001 | gtgaggagca aaagaaggga tccacctatt cagttccaaa atctaaggag aagttgatgg |
| 12061 | agaagcattc ccaggaagcc aggcaggcag acagggagtc ggagaagcct gtggacagcc |
| 12121 | tccacccggg ggccgggaca gccaagcacc cgccgccggc agcttcgctg gaaggtagga |
| 12181 | gacggcggga gggagcgaag cgaggtcagt ggcttggagg atctggggct ggggtgagga |
| 12241 | gcctactgta agaccctacc ggatggacac tagtagcagg cacgcgccgt aaagatgaaa |
| 12301 | ctggtttgtt ttgggaactt tatgtaggag gtaaatatcc agaaatctta gttgaaaaca |
| 12361 | taagtttaat gaaaatgtag gtcagttccc gagtgataag tgcgtaccag gtggtcgagg |
| 12421 | ggctggggca gtgtgcgtgt gtctcacctg aggttctcct cgagggctgg gtatcttggt |
| 12481 | gccactgaga atccctgagc ctccgcacac tggaacagag cattgggagg ccgtcccagg |
| 12541 | ctggactgta gctctccgag ggctgtgggg ataagtacat tgcggagggt gaccgaggag |
| 12601 | tagaacctgg gtgcgacaaa ggtgtctttt ataacagcct agtgtggcca gacctttggg |
| 12661 | tgtgtgtggg gaagatgagg ccccaaaagt aggaagaagc tagaatacaa agttttccat |
| 12721 | cagttttacc acgtggagct ggaagcgtta aatattttaa acaacggagg tgctgcctgg |
| 12781 | ggcggtagtt cacacctgtc ctcccagtta tttggccgag gcaggaggat cttttgagcc |
| 12841 | caggagttca agaccagctt gggcaacata gtgagaccct gtctctacaa aaaataaata |
| 12901 | tttaaaaata aaaaacaagg gcggcatgat gaggtctgtg cctaccaaca ggggaagcca |
| 12961 | gttcgcaggg gaggtggccc ggtcagagac ccattgctgt ggcccagtgg gagccagcaa |
| 13021 | ggccttgcag taggaattgc agaagggtca ggcagggaga acccgcagag cctgccgtcc |
| 13081 | gaggagattg ggggcctgtg ccaggaggag gtgttttaag tttcatcaca agaatgaacc |
| 13141 | actcggctgg gcgcagtggc tcacgcctgt aatcccagca ctttgggagg ccgaggcgtg |
| 13201 | tggatcactt aaggtcagga gttcaagacc agcctggcca acgtggtgaa accctgtctc |
| 13261 | tactaaaaat acaaaaatta gctgggcgtg gtggcatgtg cctgtaatcc cagctactca |
| 13321 | ggaggctgag gcaggagaat tgctcgaacc ttgaaccggg acccgggagg cagaagtagY |
| 13381 | ggtgagcgag atcgcaccac tgcactccag cctgggctac agagtgagac tctgtctcag |
| 13441 | aaaaaaaaaa aaataataag gagagaacca ctcgggtcag acaagaaaga atgtcagggg |
| 13501 | gatgactggc ctgctaggca gaggagctgg gcctggactg cttcacactg acacgcttgt |
| 13561 | aggatcaagg gctgaaagta ggaggctctg ggccttggac ttggaagaaa gacttggatt |
| 13621 | tgaattccca actttggaac ctcccactgg agggtggaac ccctcacagg agggcgcaac |
| 13681 | cccccaccgg aggatggaac cctgcaggaa tagcatcacc cttgggtggc agtttggaaa |
| 13741 | atgctacctg tctgtcacgg tgcttttagc tgaaagttac agaaaagctc actaaacgca |
| 13801 | gctttaacac agggatttaa tgtctcacga atgaagggtc agaggtggag cttccaggtt |
| 13861 | ggcttgttag cacaacagtg acatcaaaga ctttgcgtgt ctcttcttga ccactctcag |
| 13921 | tgtccacagc ccccgatgag tgtacagccc cccacacgca ggcaggactg catctactgc |
| 13981 | aaaggcattg ctccttgctt catattagca aggaaacctt tcccaggaag gcttcgcctg |
| 14041 | cccagcctct gcaggactca ttcaatggtg caaattcacc aggggctgga gggagctgtg |
| 14101 | aagcatggga gtgggcagga cctctgccag taacggaagt gggggaagga ggaccgcctg |
| 14161 | ttggaagggc cccagcagtg tctgtcacac cagagggtgg gggaggaagg tggacatgaa |
| 14221 | gggaaaacag cagagcaaga gtgagaacag agtttcagca gataaaatga agtgtgcacg |
| 14281 | ctctccatgc agtgacgggc tgagcgacag tgaggagcag gctgctgact cccttgccac |
| 14341 | accgtgaagg agtcccaaag gtgggacact gagtgagaca agccagagga aagaccactc |
| 14401 | atgttgcagt tccctttcca tggaatgtcc agaaaaggca catttacaga gagaagcaga |
| 14461 | tgagtggttg ctgggcagaa atgggagccg gccacagctg agcacaagag gactttggag |
| 14521 | ggtgatggaa atggttttcg acctgaatct gaacatggga agcactgcag tttagtagtc |
| 14581 | ctggtcccta agcccttcca gcccaggagc cagacctgtg agcaaacaag cctttagtga |
| 14641 | ttccaggctc tggctggaac cttgagtctt ctcagcttgg ggcatgcacc tcagggggag |
| 14701 | ccagcatcag tgtccagccc caagagcttc cctgtacgtc tcagtgagtc ttcacatgcc |
| 14761 | tccaactgcc tggacaacca cacgtgatac ctgtcctgcc aaacgtgtcc tgaacccata |
| 14821 | aaatccagag aaaagaaaat cgttttaaac tgctgaggtt tggggtaagt tactatgaag |
| 14881 | cagtagtgag aagaacagaa gggccatgat ggggagaaag tttggccata agttaatcaY |
| 14941 | tgtggcagct gttaggatat tgatgtggct tggctgtgtc ctcacccaaa tctcattttg |
| 15001 | aattcccaca tgtgggagga actgggtRgg aggtgattga attatggggg caggtctttc |
| 15061 | ctgtgctgtt ttcgtgatag tgaatgagtg tcacgagatc cgatggtttt aaaaacggga |
| 15121 | gtttcctgca taagctctct ctctgcctgc tgccatccat gtaagatgtg actcgctcct |
| 15181 | ccctaccttc cgccgtgatt gtgaggcgtc cccagccatg tggaactgta agtccattaa |
| 15241 | accttcttcc tgtgtaaatt attcagtctc tggtatgtct ttattagcag catgaaaacg |
| 15301 | gactaataca gatagatgta ttatactgtt acccctacta cttctgtata tgcttaaatt |
| 15361 | tttgcattac aaaattcaca aacatacaaa cattatagag aactaacatc acagatttac |
| 15421 | agtcacctta ttccacaatg ttggaaattc taccacaaat aaagaacttg aaaaaaaagg |
| 15481 | aggggatgtt agaaggaaat ctgagcgacc tagacctggg ctcagtcacg cctgagacag |
| 15541 | tcagatgggg tatctgagga tgagtcagtg ttaattttct cagacctggg ctccgtcact |
| 15601 | cctgagacag tcagattggg tatttgagga tgagtccgtg ttaattttct tagatgtggg |
| 15661 | aatgctatta tagttacgct aaaacacaat ccctatctaa taaatacact gcaacattta |
| 15721 | tgggtaaaat catatgtctg aaattcctta aagatactca gaatgccagg catggtggct |
| 15781 | catgctgtca tcccagcact ttgggaggcc gagacgggtg gatcacttga ggtcaggagt |
| 15841 | tcgagaccag catggccaac atggtgaaac cctgtctcta caaaaaatac aaaaaaatta |
| 15901 | gctgggtgtg gtggtgcaca cctgtaattc caactacttt ggaggctgaa gcaagagaat |
| 15961 | agctggaacc caggaagcag aggttgcagt gagccacgat catcccactg cactccagcc |
| 16021 | tgggcaatgg agtgagactc catctcaaaa aaaaaaaaga tactcaggaa aaaaacgaag |
| 16081 | aaagaacaag ataacaaaat actgattgaa gctgagtgct ctataggtgc aggtgctcat |
| 16141 | tcactattct attttcttgt gtgtttgaac actttttttt ttttttgaaa tggagtttcc |
| 16201 | ttcttgttgc ccaggctgga gtgcaatggc atgatctcgg gtctcaactc actgtagcct |
| 16261 | ccgcctccca agttcaagtg attctcctgc ctcagcctcc caagtagctg ggattacagg |
| 16321 | cgcccgccat gacgcccagc taattttttt tgtattttta gtagagatgR ggtttcgcca |
| 16381 | cgttggctag gctggcctca aactcctggc ttcagctgat ccacccgcct cggcctccca |
| 16441 | aagtgctgag atcacaggcg tgagccaccg cacctggcct cccagagcgc tgggatcaca |
| 16501 | ggcgtgagcc accacgccca gcctgtttga acacttttgt gacgttttgt tgtttgtttg |
| 16561 | ttttgagatg gagtcttgct ctgttgccca ggctggagtg cagtggcaca atcttggctc |
| 16621 | attgtgactt ccgcctcccg ggttcaagca attcttctgc ctcagcctcc cgagtagctg |
| 16681 | Rgactacagg tgtgcgccac catgcctggg taatttttgt atttttagta gagacggggt |
| 16741 | ttcaccatgt tggtcaggct ggtctcaaac tcctgacttt gtgaaccacc cgcctcagcc |
| 16801 | tcccaaagtg ccagtgttac aggtgtgagc caccacgccc ggcctgtaac attttcatga |
| 16861 | aggtagagta tacttaattg caagaaaatt ttgaaaaaga agagaaatgg aacacttcKa |
| 16921 | ttggctatta aaaaatacca tgctgagtga aaaaagccaa tcccagaagg tcacatgctg |
| 16981 | tatgattcca tttctttttt tttttttttt aggctggagt gcaaatggca cgatcttggc |
| 17041 | tcactgcaac cttggtctcc cgggttcaag caattctacc tcggcctccg gagtagctgg |
| 17101 | gactacaggc atgcgccacc atgcccagct aatctttgta tttttagtag cgatggggtt |
| 17161 | tctccatgtt ggtcaggctg gtctcaaact cccgacctca ggtgatccgc ttgcctcaaa |
| 17221 | ctcccgatct caggtgatcc gcttgcctcg gcctcctgaa atgctaggat tacaggcgtg |
| 17281 | agccaccata cccggccgtg attccatttc tgtaacattc tctgaatgac aaaattatag |
| 17341 | acagggagga cagattgttg attgctaggg ccttggcgtt ggggtaggga gtgagggggt |
| 17401 | ctgtgaaggg cagggctttg gggttggggt agggagtggg tgtgtctgaa gggcagcagg |
| 17461 | aggaagatgg gattgttggt tttggtggtg attgattaaa tggcgtagct gtacatacac |
| 17521 | tcatagtacc ggagtccatt tcctcgtgtc gataatggta ctatagttat ataaaacgtt |
| 17581 | cgctttggga gccaagcaca gtggctcacg cctgtaattt cagaactttg ggaagcctag |
| 17641 | gcaggcagat cacttgagYc cagaagttca agaccagcct gggcaacatg gtgaaacctg |
| 17701 | atttttacaa aaaagacaag aattagctgg gtgtagtgga gtgtgcctgt agccccaact |
| 17761 | aatcaggggc ctgagatagg aggatccctt gaccccatga ggttggggct gcagtgagcc |
| 17821 | aagatcgtgc cactgccctc cagccccggc gacagagtaa gaccctattt atgcttccta |
| 17881 | gcaaggaaag acagtacaca tattgaagaa tttgactgcg ttaaaatgtt aaacctctcc |
| 17941 | ccaaactctt ataaacaatt gtagaacaaa tgataagctc aaaaaaatat ttacatctat |
| 18001 | gagaggccaa agaatatcag ttgactctgg atcaggggct cggacacgtt gctgtggagg |
| 18061 | cacacatctt cctggaaggc ggtttggtaa caaggtggca ggactcaaga cagcccacac |
| 18121 | gatccctgcc cctgggaaac agccatgtgt aaccctcccc tcgagtgtgg gYggaatgag |
| 18181 | gaagagggag gtgtgtctct cctgcgatta gctcactcat ccattgagtt tgacgtcatc |
| 18241 | aaaatgcgag atgatcctgg atgggcccga cctgatcagg gaagccagac agtaaaagtg |
| 18301 | tcagaggctc tttagggccc gacctgatca gggaagccag acagtaaaag tgtcagaggc |
| 18361 | tctttagggc ccgacctgat cagggaagcc agacagtaaa agtgtcagag gctctttagg |
| 18421 | gcccgacctg atcagggaag ccagacagta aaagtgtcag aggcttttta gggcagcgag |
| 18481 | aaaacgggaa cttcattcct tgaaccggga ataaattctg cctccaaccR gaatgggctt |
| 18541 | agaagagggt cctccagctc ctcatgagaa tgcagtgcag ccggcccctg gaattcagcc |
| 18601 | tgcgtcccag ccacgctgca cctgcacccc tgacgacgaa tgcagtaaga tgataagtgg |
| 18661 | atgtgctggt ttcagccctg aagttggtgg tgatttgttg tgcagcaata gaagtacaca |
| 18721 | caatatgtac caagaacctt gagaagagtc acatttattc tttctgtttt gtttctgaga |
| 18781 | tggtctcacc caggctggag tgcagtggca ccatcttggc tcattgcact cttgacgttc |
| 18841 | tgggctcaaa cgattctccc acctcagctc cctgagtagc tggggccaca gtgcacgcca |
| 18901 | ccacccccag ctattttttg tagtttttgt agagatgcgg ttttgtcatg ttgcccaggc |
| 18961 | tggtctcgaa ctccagaggc caagcaatct gcctgcttca gcctcccaag gtgctgggat |
| 19021 | tacaggcgtg agccaccgca cctggcccaa gattaatatt tctttttttt tttgagatgg |
| 19081 | agtcttgctc tgtcgccacc caggctggac tgcaatggtg catctctgct cactgcaagc |
| 19141 | tccgcctccc gggttcatgc cattctcctg tctcagcctc ccgcatagct gggactacag |
| 19201 | gtgcctgcca ccacgcctgg ctaatttttt gtattttttt tttttttaag tagagatggg |
| 19261 | gtttcactat gttaaccaga atggtcttaa tttcctgacc tcgtgatccg cccgcatcgg |
| 19321 | cctcccaagg tgctgggatt acaggcgtga gccactgcgt ctggcctaat atttctaaat |
| 19381 | gaagaaacta tactYtgagg aagatgaaat aagtcttgtt ttagactaga aaatttcaat |
| 19441 | agtgtaaata cgttaacttc tgtaaaatca gtcaacacca agtcaaaaca caaaagatct |
| 19501 | tttgggaata tgagcagatt attcaaaggt tcatctaaag aataaaaatg aggccaagtg |
| 19561 | tagtgattca cgcctgtaat ccYggcactt tggaggccga ggcaggtgga tcacttgagg |
| 19621 | tcaggagttc aagaccagcc tggccaacat gatgaaagcc catctctact aaaaatacaa |
| 19681 | aaattagcta ggcgtggtga tgggtgcctg tagtgccagc tacttgggga ggctggggca |
| 19741 | ggagaatcac ttgaacctgg gaggtagagg ttgtagtgag ccaaaattgt gccactgcac |
| 19801 | tccagcctgg gcgacaagag tgagaatcca tctcaaaata aataaatgaa taaaaatgaa |
| 19861 | agcagtagga aacatctgga aaaccagagg catgaggggg tcgggccctc tgatactcaa |
| 19921 | gtaggttaga gtaaaaaaaa gccagatatg gtgatgtggg aatatatggt cccataggtg |
| 19981 | ggatctgtgg ttccagctac tcagtgaggt ccgaggatca cttgggcctg ggaggttagg |
| 20041 | actgtagttt gctatgatca catccacgaa tagccactgc gctccttcct gggctacaga |
| 20101 | gtgagaccct tctctaaaaa tggaagaaaa tggccggcac agtggttcat ggctgctgta |
| 20161 | atcccagcgc tttgggaggg tgaagtgggagaataacttg agcctgagag ttcgagacca |
| 20221 | gcctgggcaa catggcaaaa ccgtgtctct acaaaaaatt tcaaaattag gtgggtgtgg |
| 20281 | tggtgtgtag ggtccagccc cacagggtcg gtgggttttc tccccatgtg cggagatgag |
| 20341 | agagcgtaga aataaagaca caaggcaaag agataaaaga cagctgggcc cgggggacca |
| 20401 | ctaccaccaa gccgtggaga ccggtagtgg ccccaaatgc caggctgcac tgatatttat |
| 20461 | tggatacaag acaaaggggc agggttagga gtgtgagcca tctccaatga taggtgaggt |
| 20521 | catgtgggtc atgtgtccac tggacagggg gcccttccct gcctggcagc caaggcagag |
| 20581 | agagaggaga cagagagaca gcttacgccg ttatttcagc atatcagaga cttttagtac |
| 20641 | tttcactaat ttgctactgc tatctaaaag gcagagccag gtgtacagga tggaacatga |
| 20701 | aggtggacta ggagcgtgac cactgaagca cagcatcaca gggagacgat taggcctctg |
| 20761 | gataactgcg ggcaagcctg actgatgtca ggccctccac aagaggtgga ggagcagagt |
| 20821 | tttctctaaa ctcccctggg gaaagggaga ctccctttcc tggtctgcta agtagtgggt |
| 20881 | gtttttcctt gacactgacg ctactgctag accatggtct gcttggcaac gggcgtccta |
| 20941 | ccagacgctg gcgttaccRc tagaccaagg agccctctgg tggccctgtc tgggcataac |
| 21001 | aaaaggctca cactcttgtc ttctggtcac ttctcaccat gtccccccag ctcctgtctc |
| 21061 | tgtatgacct ggtttttcct aggttatgat tgtagagcga ggattattat aatattggaa |
| 21121 | taaagagtaa ttgctacaaa ttaattaatg atattcatat ataatcatgt ctgtgatcta |
| 21181 | gatctagtat aactattctt attttatata ttttattaca ctggaacagc tcgtgccctc |
| 21241 | ggtctcttgc ctcggcacct gggtgacttg Ycgcccacag tggtgcgtgc tggtggtccc |
| 21301 | agctacttgg aaggctgaag tgggaggatc atttgagccc aggaagttga ggcttcagtg |
| 21361 | agccatgatc atgccacagc atttcagcct gggccacaga ccttcaagat cctgtctcaa |
| 21421 | aaagaaaaag ggRtatcKct cagtgcttag tgcagctgtg ggtcattcgg cagctgacac |
| 21481 | agatgaggct atgtaacgca gcactgcagg cagaggggga ggccaaggcc tgaatacccc |
| 21541 | gtctgactag ccttaggcag gcgacttggc ctctctgcct cccccatctg gttagccttg |
| 21601 | ggcagtcgag ttggcctctg tgtctcccct gtttgactag ccttgggcag atggcttggc |
| 21661 | ttgtctgtac ctcagttttg tctgaaatat gggYtgtcag agacetatag cagaagctgc |
| 21721 | tgtgtgtcat tattcactgt tctagggctc agaacaggca cgcgataaaa gccagcaccg |
| 21781 | ttggccgggc gcattggctc acgcctgtaa tcccagcact ttgggaggcc aaggcaggta |
| 21841 | gatcacctga ggtcgggagt tcaagaccag cctggccaac atggagaaac cctgtctcta |
| 21901 | ctaataatac aaaaattccc caggtgtggt ggcaggagcc tgtagtccca gttgctcagg |
| 21961 | aggctgaggc aggagaatca cttgaactca ggaggtggag gttgcagtga gccgagatca |
| 22021 | caccactgca ctccagcctg ggtgacagag cgagactccg tctcaaaaac taagaagaag |
| 22081 | attaataata aaccagcact gccattgcca gcagtccatc ctacctcgca tccaggttcg |
| 22141 | tgagctacca agtgtcccca caaagctgta aaacaRcatg accactgtag ttacgtttct |
| 22201 | gtaaaaaggt gactgagtct tctaaccctg accacacata tgtgtgcaga gagagccagc |
| 22261 | tccatcctgc tctcccagaa gcctgaacag ctgtgcccta gctcgtagca gtggcactcc |
| 22321 | aggaaggggg cgggcggctg tgcccgagct cgtagcagtg gcactccagg aagggggcgg |
| 22381 | gcggctgtgt ctgagctcgt agcagtggca ctccacgaag ggggtgggcg gctgtgcccg |
| 22441 | agcttgtagc agtggcactc caggaagggg cggccggcca gccaggagag cagccggtga |
| 22501 | aggagcactt ttgctttgta cgtggcaacg ctcggcaggc tcagcagttt cacaggtctc |
| 22561 | aggcgttcct tgtaacctta acaggtttaa gaaaatgtga gcaacatcag acatgtccat |
| 22621 | ttctagaaca ttctaatgga tgaagttctg tacagagggc tttagcagaa ttagagacct |
| 22681 | ttggagtgga aatgatgtct ctgaaccttg agctctgagg cggttcctgc ctggacgtgt |
| 22741 | gccctccctc cttcctttgc tggtcgccct gacaccatcc tgccacggat agtggaagtg |
| 22801 | tttctcctgt tttcttccct gaaaaagggt gtccagcatc agcaaccttc atggctgagt |
| 22861 | tgcacatccc ttaagccctg taatgttcat ggacagtttt gatcactttt ggactcaccc |
| 22921 | attttttctg ctaaccatac atcttaattc ctttactctg accaatggca ggaggtgtgt |
| 22981 | gcatcgtgga aggagctttc cacacatcta ctaaatgcct ggatgttttt tcctctagag |
| 23041 | aaacctgatg taaagcaaaa gtccagcagg aagaaagtgg tcgttccaca gatcatcatc |
| 23101 | acgcgagcgt cgaatgagac gctagtcagt tgcagttcca gcgggagtga ccagcagaga |
| 23161 | accattcggg agccggagga ctggggcccc taccggcggc acaggaaccc cagtacagca |
| 23221 | gacgcctata attcacatct caaagaataa acaagcacct ttgcatggca ctcgctactc |
| 23281 | cctgcgatag gcgtctcggg aacagccgcc tcaccctgtg agaagccgag gccccttctc |
| 23341 | cagtgctctc ggggaggttg caccaggccc accccaccct gtgagaaact gcagccccct |
| 23401 | tctccagtgc tttcggggag ggtgcaccag gcccgcccca ccttgtgaga agccgtggcc |
| 23461 | cccttctcca gtgctctcag ggagggtgca ccaggcctgc ccccgccgtg agaaactgca |
| 23521 | gYccccttct ccagtgctct cggggagggt gcaccaggcc caccccaccc tgtgagaacc |
| 23581 | tgcagccccc ttctccaatg ctctcgggga gggtacacca gggttgcccc accctgtgag |
| 23641 | aagccaccgc ccccttctcc agtgctctcg gggagggtgc accagggctg ccccacgctg |
| 23701 | taagaagccg ccgccccctt ctccagtgct ctccagcccg ccccaccctg tgagaagcca |
| 23761 | tggccccttc tccagtgctt ttggggaggg tgcaccaggg ccaccccacc ctgtgagaag |
| 23821 | ccgtggcccc cttctccagt gctttcgggg agggtacacc aggcccgccc caccctgtga |
| 23881 | gaagccgtgg cccccttctc ccgtgctctc agggagggtg caccaggcct gcccccgcct |
| 23941 | tgagaaactg cagccccctt ctccagtgct ctgggggagg gtgcaccagg cctgcccacc |
| 24001 | ctgtgagaaa ctgcaccccc ttctccagtg ctctcgggga gggtgcacca ggcccgcccc |
| 24061 | accctgtgag aagccgcagc ccgttctcca ttgccatttg gtcctgcctt ccccagagct |
| 24121 | gctagcagct cggtcgaggc aagagtgact ggctcagaga gcgagctttg tggtattgat |
| 24181 | aaatgaataa attagtatgt tcatgttgga agtggctggc tctgcagagt gatggacagc |
| 24241 | aggtggcatc ttacattgtg atttgtggtg cacagttaaa acacttccct tcgtttcaga |
| 24301 | aggagacaat gtgaatactt ggaaattata gtgaataaga tgcctcccaa acttgaggaa |
| 24361 | aaggtcagat tctaagagct aaacgttaag atgtaatgtt ccagaacaaa gaggcccaga |
| 24421 | gtcactgaac ggggccccag ggctcctagc ttgagtgtaa tctgagtcag cttatgaaaa |
| 24481 | ggccccaaag aagaaagtgg aatgtgtgct gggcacatgg gacagccatg gcactgagac |
| 24541 | tttggaagtg gccttgccag cgctgtcctg tctttctggc ccacagatga gggctcctga |
| 24601 | ttgtggaagg ccacggaagg gacctgaaca cgatggcacg agacaagtta tctcctgtga |
| 24661 | ctgctcctct ccagcaaccc tcagctcagg ctagactttc cccctgtgtt gagaatgccc |
| 24721 | tgccctgtgt ggaagcctga tgcatttagt gacagtgtgt tagctttgtt agtttctagt |
| 24781 | ctggccagca ggctttccat gtgatctgcc attggcttgc ttcagtggcc agtggagcca |
| 24841 | ggggtgtcca ccagagtctt ggacacagac ttctgaggtg gcagcgggag ggagcctgct |
| 24901 | ccctgccaca cacgtacaat ttctaatcca tacaggccta tgtagtatat acacacatac |
| 24961 | gcagagcctt aggaattgtg aaaggccaat caaaacttgg ggccctaatt tcactctgcc |
| 25021 | aaaagaaaaa aaaaaaaagc tgaaagatga gtcacccaag aaactgcttc ctttttgttc |
| 25081 | ctaagcagag agctaccaat aaaaagttaa agtttctcca taggtagcta ctctgttcac |
| 25141 | cttctgtgaa gtgccaaatt ttttttttat tattatactt taagttttag ggtacatgtg |
| 25201 | cacaacgtgc aggtttgtta catatgcata catacgccat gttggtgtgc tgcacccatt |
| 25261 | aactcgtcat ttacattagg tatttctcct aatgctttcc ctcccccctc ccccctaccc |
| 25321 | cacgacaggc cccagtgtgt gattttcccc ttcctgtgtc caagtgttct cattgttcag |
| 25381 | ttcccaccta tgagtgagaa catgcggtgt ttggtttatt ttacttgcga tagtttgctg |
| 25441 | agaatgatgg tttccagctt cacccatgtc cctgcaaagg acatgaactc atcctttttt |
| 25501 | atggctgcat agtattccat ggtgtatatg tgccacattt gcttaatcca gtctatcact |
| 25561 | gatgggcatt tgggttggtt ctaagtcttt gctattgtga atagtgccgc attaaacata |
| 25621 | ggtgtgcatg tgtctttata gcagcatgat ttataatcct ttgggtatat acccagtaat |
| 25681 | gggatggctg agtcaaatgg tatttctagt tctagatccc tgaggaatcg ccacactgtc |
| 25741 | ttccacagtg gttgaactag tttacagtcc caccaacagt gtaaaagtgt tcctatttct |
| 25801 | ccccatcctc tccagcacct gtttcctgac tttttaatga tcgccattct aactggtgtg |
| 25861 | agatggtatc tcattgtggt tttgatttgc atttctctga tggccagtga tgatgagcat |
| 25921 | tttttcatgt gtctgtaggc tgcataaatg tcttcctttg agaagtgtct gttcatatcc |
| 25981 | ttcacccact ccatcaaaaa gtgggtgaag tgccaattta ctgagcacga gacaaataca |
| 26041 | tcattgactt cttttgtctt gtggattcaa ggatgtgtcc acgcccgccc tccttcccct |
| 26101 | ccagctcctc cccttgaaat actgaagccc tcaacatcat ctttggagga aggtacagag |
| 26161 | ttggctctca cgcgcacatc cttaaccttg gcctaataaa cctctaaggt ggctgagacc |
| 26221 | tgtctcagat acttgttggc ttacaaattg accaccatgc agggactcag agtggaggtg |
| 26281 | gccctgccct ttggcaggtt tcctcctggt gcttaggacc agtttgggcc ttctttattg |
| 26341 | ctcaggacag taggacaatt tgctgagatc cgggagccct accaccctcc agagaatctc |
| 26401 | tgatctcccg aaacttggtt gaggtcgaaa gtttattttg cgcacaatgc cttttccttt |
| 26461 | tccggtgttt tacttgcttc caacaaagaa ggcgagtttt cctgcttccg tgatgatggg |
| 26521 | gagcaggaga cttccctgga gtttcagctc ggaaacaact cgagtttttc ctgattccac |
| 26581 | gatgggggag agcagtctaa agcctgggcc ccattcctag gtaagtggct gaattggggt |
| 26641 | tctgtcttgg aagttctcct taatgactaa agctaacatt aacgactggc tggtcttaac |
| 26701 | ttcttactat tagcatgctc agtaagtgta taaattgtgg gattgtttgt tttgcttaac |
| 26761 | tgctttttgt ttgtttctgt gtttgctatt tttgctgttt cagtcttttt cccgttggat |
| 26821 | ttgacgaatt ctgttagact tggtcaaatc tgaagcaaac ttccaagttg tgggaaacaa |
| 26881 | gcctctgaat tagctgaatt cccacagctg ggaaaaagga aaaaaagcca gaaaaaggga |
| 26941 | aaaaagattt tgaccacgtg atgggcttta tttgcataac gaggccacgt ttttccagcc |
| 27001 | aagccacact cgaagagtaa gtgccgtcac ctcacgctgc agttcggtag gtagagtccg |
| 27061 | ccatttttcc accaggacag cccgggtttg gttcctacat cctttctggt ttgaaatttg |
| 27121 | tgttactttt gaaatatcag cagtttgtcc cggctgaaat aaggtagtga gatttaaaga |
| 27181 | tttaaaagga ttttcttttt ttttgagacg gagtctcgct ctgtcgccca ggctggagtg |
| 27241 | cagtggcacg atcccagctc actgcaacct ctgccgccca ggttcagcca ttctcctgcc |
| 27301 | tcagcctccc aagtagctgg gattacaggc acttgccacc gcacctggct aatttttgca |
| 27361 | tttttagtag agacggggtt tcaccacctt gaccaggctg gtcatgaact cctgatcttg |
| 27421 | tgatccaccc tccttggcct cccaaactgc tgggattaca ggcgtgagcc actacgccca |
| 27481 | gccaagagga tttttaaaag ggcttaatgg tcaaaagtaa acttaactaa aagctaatat |
| 27541 | cggatatatt ccaacatagt agcttaatta agtccgtatg ccacatacac atatacagac |
| 27601 | ccctgtgtag aaaagaaggc accagactcS tttttgggga gaaacttctg tttttcttat |
| 27661 | ggaactccaa gagtggaaac agacacgttc ctctcagatc ttaaattgct tgctattgta |
| 27721 | ttgtgttacc tgattttttt ttactaaaat agttgacaca ggactttttg gggtgctgct |
| 27781 | tctctagctg gaggcctctg tggccggtgg tgcccctacc cgggtctcct gggctccagg |
| 27841 | gttggccctg gagtaccccg cccacctagc ccggccgtgg gatccgccct cagcccatgg |
| 27901 | ctggactgcg catgcgcacg ccgcaacccg cttcagcctt gagcactgga gtcYggacaa |
| 27961 | gaggagtgtg gtggcgcaca aaacgcccaa agatgccagg aacggcagag ccccaagggt |
| 28021 | gtcacagctc gattgttccc cccgcctgcg gtctggtgag tgggggtgtg ttacagtgtc |
| 28081 | accccccctt tttttttttt tgagacagag tctcactctg tcactaggcc agagtgcagt |
| 28141 | ggcacagtct cggctcactg caacctccgc ctcctgggtt caagcgattc tcctacctca |
| 28201 | gcctcccaag tagctgggag tacaggtgcc tgccaccaca cctggctaat tttttttttt |
| 28261 | tttttttttt ttttttttga gacagagttt ctctcttgtt gcccaggctg gagtacaatg |
| 28321 | gcacgatcgc agctcactgc aacctccacc tcccaggttc aagcgattct cctgccttag |
| 28381 | cctcctgagt agctggaatt acaggcatgc accaccatgc ccggctaatt ttttgtattt |
| 28441 | ttagtagaga cagggtttct ccatgttggt caggctaatc ttgaactccc gaccttaggt |
| 28501 | gatccacccg cctcagcctc ccaaagtgct aggattacag gcatgagcca ccacacccag |
| 28561 | ccagtttttg tgttttcagt agagatgggg ttttaccatg ttggccagga tggtcttgat |
| 28621 | ctcttgacct tgtgatccgc tcacgtcagc ctcccaaagt gctgggatta caggtgtgag |
| 28681 | ccaccaggcc tggccgtcac cccctttttg aatctgtgat tcagcaagtc ccaaatgctt |
| 28741 | ctcccatgtc caagaaaaat gaaggtatgc agacaaccgg agggtgagca ggacaacgtt |
| 28801 | ttgttgagca acagaacagt tctcagtgaa gaggggagcc aaagtgggca gcccctactc |
| 28861 | aaagttgggt acgcccccac tgaagtcccc caccatgcac agcctataat tttatctttg |
| 28921 | acttggcatt cttttttttt tttttgagac agagtcttgc tctgttgccc aggccggagt |
| 28981 | gcagtggtgc gatctcggct cgctgcaacc tccgcctcca gggatcaagt gttctcctgc |
| 29041 | ctcagcctcc tgagtagctg ggattacagg tgcccgccac tacacctggc taattttttg |
| 29101 | tttgtttgtt tgtttgtttt ttgagacaga gtcttgctct gttgcccagg ctggagtgca |
| 29161 | gcagcgcaat ctctgctcac tgcaagctcc acctcccggg ttcacgccat tctccggcct |
| 29221 | cagcctcccc agtagatggg actacaggcg cccaccacca cgcccagcta attttttgta |
| 29281 | tttttattag agatggggtt tcaccgtgtt agccaggatg gtctcaatct cctgaccaca |
| 29341 | tgatccgccc acctcggcct cccaaagtgc tgggattaca ggcgtgagcc actgcgccca |
| 29401 | gcctaatttt tgtattttta atagagacag ggtttcacta tgttggtcag gctggtcttg |
| 29461 | aactcctgac ctcatgatct gcctgcctcg gcctcccaaa gttctgggat tacaggtaac |
| 29521 | attcattttt aatgtccctc taacacaccc agactctccc tctgtatttt gagatgtaaa |
| 29581 | ttttgctatc tgatttttca cctaagagtt cttcctttgg tatgtcaatt tagggctatc |
| 29641 | aagctgaaaa ttgcctaaga caatgaaaca ggttatcaag aaattggaag tttaggccgg |
| 29701 | gcgcaatggt tcacgcctgt aatcccaaca aattgggagg ccaaggcggg cagatcactt |
| 29761 | gaggtcagga gttcgagacc agcctggcca agatggtgaa accccatctt tactaaaaat |
| 29821 | acaaaaatta gctgggtgtg gtggcacgta cctgtaatcc cagctacttg ggaggctgag |
| 29881 | gcaggagaat cgcttgaacc tgggaggcgg aggttgcagt gagctgagat tgcgccattg |
| 29941 | tactccagcc tgggcaacaa gagcaaaact ccatcttaaa aaaaaaaaaa tacaaaaatt |
| 30001 | agccgggcgt ggtggcaggt gcctgtaatc ccagctaccc aggaggctga ggcaggagaa |
| 30061 | tctcttgaac ccaagaggtg gaggttgcag tgagttgaga ctgtgccact gtactccagc |
| 30121 | ctgggtgaca aagtgagact ccaactcaaa aaaaaaaaga aaaagatcga aagaaagaaa |
| 30181 | ttgtaagttt aaaatagtag aaaaaaaaga aggggtctta tgcatcttct gtgtgtcgaa |
| 30241 | tgcatctatg tatctatgag tcatgtatac aatgtttcac taccaaaaat atataaaaga |
| 30301 | gttttaatta attgggttaa aggaaaaaaa agcacttaaa tactgtatca gaaaaatata |
| 30361 | aacttttttt tttttttttt tttttggaga cagaatgttg ctctatcacc caggctggag |
| 30421 | tgcaggggca cgatctcggc tcactgcaag ctccgcctcc agtgttctca cgccattctc |
| 30481 | ctgcctcagc ctcccgagta gctgggacta caggagcccg ccaccacgcc cggctaattt |
| 30541 | tttgtatttt tagtagagac ggcgtttcac cgtgttagcc aggatggtct cgatctcctg |
| 30601 | accttgtgat ccccccgcct cagcctccca aagtgctggg attacaggtg tgagccacca |
| 30661 | tgcctggcca gaaaaataga aactttaagc ccaaatgctt tttcaggttc atgtgactta |
| 30721 | agtaaatcct taaaaaataa gctagtttta aaattattgg taaaataaaa tagaaatgcc |
| 30781 | ttcagaattg tcaacataca ttattgttta gatttattgg taaaggagtt caatatttat |
| 30841 | cattacgaga tactgtaagt tgtcaaaatt tggcatgagg gttataaggc ttgcaacaca |
| 30901 | aaagggaatt atgtttgact aatttcttga taaataaggc atttaatatt gtttaatgga |
| 30961 | aagagataaa tcctgagttg ctggcaaaaa aaaaaagagg aacatttatt taaccataag |
| 31021 | gttcttactt aggtaaacac ctaaaattca caggctataa aaatggttag gccaggtatg |
| 31081 | gtggctcacg tctgtaatcc cagcaatttg ggagtctgag gtgggcagat cacctcagat |
| 31141 | caggaattca agaccagcct ggccaacatg gtaaaacccc gtctctacta aaaatacaaa |
| 31201 | aattagctgg gcatggtggc aggtgcctgt aatcccagct acttgggagg ctgaggcagc |
| 31261 | agaatcactt gaacccagga ggcggaggtt gcagtgagcc aagatcacac cattgtactc |
| 31321 | caacctgggc aaaaagatca aaagaaaact ccatctcaga aaaaaaaaaa aaaagaaaat |
| 31381 | ggttaacaga gaaataactt taaatgaaga ctatcacagt tttctttcct ttttcttttt |
| 31441 | tttttttgag aagagttcac tctgtcaccc aagctcgaac gcaatggcgt gatcttggct |
| 31501 | caccgcagtc tccgcctccc aggttcaagc tattctccca cctcagcctc ccaagtagct |
| 31561 | gggattatag gtgcccacaa ccacgcctgg ctaatttttt tttttttttt ttttgagaca |
| 31621 | gagtctcgct ctgtctccca ggctggagtg cagtggtgct atctcggctc actgcaagct |
| 31681 | ccgcctcccg ggttcatgcc attctcctgc ctcagcctcc tgagtagctg ggactacagg |
| 31741 | cacctgccac cacgcctggc taattttttg tatttttaat agagatgggg tttcaccgtg |
| 31801 | ttagccagga tggtcttgat ctcctgacct tgtgatccgc ccagtctggc taatttaaaa |
| 31861 | aaaatatttt tatagtaggc tgaggcgtga gaatggcatg aatctgggag gcggagcttg |
| 31921 | cagtgagccg agactatgcc actgcactcc agcctgggca acagagtgag acaccatctc |
| 31981 | aaaaaaaaat ttattttaaa taaacaaata aataattaca gatgtaaaga tagttataag |
| 32041 | gagatatttt tagtatgaaa agttaaaagg aaaataattt catatgagaa agaatcttgt |
| 32101 | gtggtaaatt tttgttctaa aatggctagt tattttggaa agagggatgt tcagaataaa |
| 32161 | acaggaagtc caagtatatc ataaatggtt tgtgtaagtt ttgataaagt ttgtaaaaag |
| 32221 | cttttttttt ttttttttaa gatggagtct cgctttgttg tctgggctgg agtacagtgg |
| 32281 | tgcaattttg gcctatgtca gattaacaag gctttcttgg agcattaacc cactctttaa |
| 32341 | ttaaaaaaaa aaagttataa aaatgtttat ggaaattatc ttttttgttt ttgaggcaga |
| 32401 | gtttcgctct tgttgcctaa gttggagtac agtggtgtga tctcagctca ctgcaacctc |
| 32461 | tgcctcctag gttcaagcaa ttcttctggc tcaggctccc aagtagctga gattacaggc |
| 32521 | gcccacaacc atgcctggct aatttttgta tttttggtag atacgtggtt tctccatgtt |
| 32581 | ggccaggctg gtctcgaact cctgacctca ggtgatcgac ctgcctcggc ctcccaaagt |
| 32641 | gctgagatta caggtgtgag ccaccgcacc cagccttatg gccttatttt ttttgttttt |
| 32701 | ttttagatgg agtcttgttc tgtcacccag gctgaagtgc agtggtgtga tctcggctca |
| 32761 | ctgcaacctc cacctcccag gttcaagcag ttctcctggc tcagcctccc aagtagctgg |
| 32821 | gatcacagac gcacactacc atgcctggct actttttgta tttttaggaa agatgaggtt |
| 32881 | tgccatgttg gtcaggctgg tcttgaactc ctgacctcaa gtgatccgcc tgccttggcc |
| 32941 | tcccaaagta etaggattac aggcgtgagc caccacaccc agcctgaaaa ttatatcttg |
| 33001 | tgctcaagat gattacaatt taatagattt gtttataaaa ttttagagag atttaattgg |
| 33061 | ccttatgctg tctttatctt ttgagaagaa attccctcaa agaataaagg tttctacatt |
| 33121 | tttttttgaa atctttgagt tatctcagca ccataggaaa ggtaaaaaca gaaaaaaaaa |
| 33181 | aagaaaataa aaaaagaaat ctgtgagtta tcactgtggc taagtgaatg acttatttta |
| 33241 | caatgacctg tgatcctatt tagtgatagc aactgtttta aaactttttc taggccaggc |
| 33301 | acggtggctc acacctgtaa tcccagcact ttgggaggct gagatgggca aatcaagagg |
| 33361 | tcaggaaatc aagaccatcc taactaacat ggtgaaaccc tgtctctact gaaaatacaa |
| 33421 | aaaattagct gggcgtggtt gcaggcgcct gtagtctcag ctgcttggga gactgaggca |
| 33481 | ggagaatggc gtgaacccgg gaggcagagc ttgcagtgag ccaagattgc agcactgcac |
| 33541 | tccagcctgg gtaacagagc gagactctgt ctcaaataaa aacaaaaaca aaacaaaaca |
| 33601 | ctttttatat ttgacaaact tKccaaaatc aaattctttc ttcagtcctc attaattttt |
| 33661 | gtgtgtgtgt gtctgatgga gtctcactct gttgccccag ctggagtgca gtggcacaat |
| 33721 | ctcggctcac ttcaacctcc acctcccagg ttcaagcgat tctcctgctt cagcctccca |
| 33781 | agtagctgga actacaggtg cccgccacca cacctggcta atttttatat atttttttct |
| 33841 | ttttgagata gagtcttgct ctgtcaccaa ggctggagtg cagtggcacg atctcagctc |
| 33901 | actgcaagct ccgcctcccg ggttcatacc attctcctgt ctcagtctct tgagtagctg |
| 33961 | ggactacagg cgcctgccac cacgcccagc taattttttt tttttgtatt ttttagttga |
| 34021 | gacagggttt cactgtgtta gccaggatag tctccatctc ctgacctcat gatccacctg |
| 34081 | cctcagcttc ccaaagtgct gggattacag gcgtgagtca cctgcctcag cctcccacag |
| 34141 | tgctgggatt acaggtgtga gccactgcgc caggcctccc aaggtgttgg gattacaggc |
| 34201 | gtgagccacc gctccgggcc tcccacagtg ctaggattac aggtgtSagc tgctgcacct |
| 34261 | ggcaattttt tgatattagg tcccctgaag tccaaaaaga gatatatggc ttatttggta |
| 34321 | taatgaaatc atacaggaag cattgttaaa ggtgaaatgg tgtttttctt gggattatat |
| 34381 | ttatataaat gtgttgttag tgtgtgttcc aaaattgtat gagatgcctg tgattctgat |
| 34441 | acatcttagt atatggtatt agtagtaatt atgattatta tgtaaaattg ttgtatgcca |
| 34501 | cataagtaac caaatttgct tgtcaattat gtcttttttt tttctttttt ctgagacaga |
| 34561 | gtcttgctct gtggcccagg ctggagtgca gtggtacagt cttggctcac tgcaacctct |
| 34621 | gcctcccagg ttcaagcgat tctcatacct cagcctccag agtagctgga ctgcaggcgc |
| 34681 | atgctaccac ggccagataa tttttgtatt tttaatagag acggggtttc tttttttttt |
| 34741 | ttttccctga cagagttttg cttttgttgc ccaggcttgg agtgcaatgg cgcgatcttg |
| 34801 | gctcactgca acctccactt cctgggttca agcaattctt ctgcctcagc ctcccaagta |
| 34861 | gctgggatta caggtgtaca ccaccatgcc tggctaattt tgcatttttt tagtagagac |
| 34921 | atggtttcac catgttgatc agggtcgtct caaactcctg acctcaagtg atccacccgc |
| 34981 | ctcagtctcc caaagttttg ggattataaa tgtgcgccac tgcacccggt caagacaggg |
| 35041 | ttttgccatg ttagccaggg aggtctcaaa ctcctgacct caagttatcc gccctgcttg |
| 35101 | gtctcccaaa gtgctgggat tgcacacatg agccactgtg cctggcctca attatgtctt |
| 35161 | taactgtggc tgttctaaga cttttgtcat ccacaattgt tgttttactt ttatcctttc |
| 35221 | acagagcagt ttataatcag ctatagaact ctgaggtact cttcaatcta agtttctgat |
| 35281 | cacttttgag attgtgccat tggaatagag caaaaaactt ccaagactct cattgagagc |
| 35341 | tgctatattc atgaggattg ttcatccaat attgggcaaa atgagagtta attttatgga |
| 35401 | ctaaactaat ataagactga aataatcttt ttatgttttt gcttaaaaca ttactgattc |
| 35461 | tttctgttct ttcagaacca agaaaacttt tttttttttt tttttgagac agaatctcac |
| 35521 | tctgtcgccc aggctggagt gcagtggcgc catctcggct cactgcaagc tccgcctccc |
| 35581 | aggttcacac cattctcctg cttcagcctc ccaagtagct gggacztacag gcgcccacca |
| 35641 | ccgcgcccgg ctaatttttt gtatttttag tagagacagg gtttcaccgt gttagccagg |
| 35701 | atggtctcaa tctcctgacc ttgtgatccg cccacctcgg cctcccagag tgctgggatt |
| 35761 | acaggcgtga gccacggtgc ccggctggta acttttttaa ctttgagcta tttgcagctt |
| 35821 | ttagcaattg agtaaagtac actcctgtga ggaagatttg gaacatattt ctgttttctt |
| 35881 | ttttgttttt tatattttgc ttgggtagca tatttctttc tgatttctcc aaaaattggg |
| 35941 | aactatctgt gagcactatt taaaaaaaaa aaaaaaactt ttccctctcc cttcccctat |
| 36001 | ttgtattcta atgtcaatat aatgatgtgt gcacgtgcag taacaatctg ttttctcttg |
| 36061 | taataggaca tagagacact gcatatttta ccaaggcttt gactggaatg gcatgctttc |
| 36121 | aaatataaac agacggcttt aaggaatcag agttgaccta tagtgcccat caaagccctt |
| 36181 | gggaaaactg gcttcatact gtatccaccc agtggattct tcttgcccag atggagccca |
| 36241 | tgtatcaagg tagggaaact gcaatagaga aagggtttaa ttcacacaga gacaagtgaa |
| 36301 | tgggagacca gaatttgatg attactgaaa tcagtctccc caagaatttg gggactaggg |
| 36361 | tgttccaaag gtagtttggg ggaaggggtg gggtagcttg gcagtgggtg cttgctgctg |
| 36421 | atgggttgtg ggggcggggg gccatcgtag ggtgtgagac aggatcctcc cgtgtgctga |
| 36481 | accgcttctg ggtggggctg caggagtagt tggcaggtcc aggtggagcc atcatcagtc |
| 36541 | agtgggtcct gttggagccg taggtatcag acatgcaaaa aacccttgSg aggtactgca |
| 36601 | aaaggccaat cttaggttac aatagcattg ttatctgcag gacctctgga ataatggctg |
| 36661 | ataattgttt atgtctacat cttagccaaa ttcaggctcc tctcctctcc ctagcctggt |
| 36721 | ggcccctcat tagttttaca aatacagttg agttttgggg aggggctatt atcatgtaaa |
| 36781 | ctataaacat ctcccaaagc tagcccaaga ataactgaag gcagcttgaa ggctaaaggt |
| 36841 | caaagggggt ttggctacat cagatcttcc ccaattggca taattttctc aatgatacaa |
| 36901 | tttttgtggc cgggctcggt ggctcacgcc tgtaatccca gcactttggg aggccaaggc |
| 36961 | gggcagatca cgaagtcagg agattgcaac catccttgtc aacacagtga aaccccgtct |
| 37021 | ctactaaaaa tacacaaaaa attagccggg cgtggtggcg ggcgtctgta gtcccagcta |
| 37081 | cttgggaggt taaggcagga gaatggcgtg aacctgggag gcggagcttg cagtgagccg |
| 37141 | aggtcgcacc actgcactcc agcctgggcg acagagcaag actccgtctc aaaaaaaaaa |
| 37201 | aaaaaaaaaa aagcctacat ggtaaatatt tattcttgct acagtttata caaataacca |
| 37261 | ggccaagtac aataagacta aaacttattt tgaaaacaaa tcagtactac ctgatttcac |
| 37321 | tttggtaaaa ataggagact agagagagaa aaattatgtt tcaaaataaa ctatagtata |
| 37381 | cctgttatta gattctagcc ttgcctaagg tttttcaact tttattattt tctctttttt |
| 37441 | tgtttgttta tttgtttttt cagacagggt ctcactctgt cacccgggca gtggtgtggt |
| 37501 | atcagttcac tgcaacctct gcctcctggg ttcaagtgat tctcgtgcct cagcctcccg |
| 37561 | agtagctggg attattggtg tgcaccacta tggccagctc atttttttgt atttttagta |
| 37621 | gagatgcgtt tcaccatgtt ggccaggcta gtctcgaact cctgacctca tgatccaccg |
| 37681 | gcctcggcct cccaaagtgc tgggattata ggcatgagcc actgcgccgg gccaaagccg |
| 37741 | tgcttttctt aaagctctgc gaacgaaagc tagacaactt aaacctcaga agagaataat |
| 37801 | ggtacctatt tatatacata aaccactttc acgcctgcct gctgtgtatg gacttcagag |
| 37861 | gaacacagcc tacgtcagtt ttctaggatt gttctctcca cgtgctcttt gttttctttc |
| 37921 | tttcttcttc cctctatttt cctccctccc tcccttcctt ccttcctttc acaaagtctc |
| 37981 | actctgttac ccaggctgga gtgcagtggc acgattttgg ctcactgcaa cctccgcctt |
| 38041 | ccgggttcaa gcgattctcc tgcctcagcc tcccgagtac ctgggactac aggcaaccac |
| 38101 | caccacaccc agctaatttt gttagtttta gtagagacgg ggtttcacca tgttggccag |
| 38161 | gctggtctcg aactcctgac ctcaagtgat ctgcctacct cggcctccca aagtgttggg |
| 38221 | attataggca tgagccactg tgcctggtca caacacctgc attttctttt ttttttgttt |
| 38281 | ttttttttga gacagagtct cactctgtca cccaggctgg agtgtagtgg tgtgatcttg |
| 38341 | gctcactgca agctccgccc cgtgggttca caccattctc ctgcctcagc ctcccgagta |
| 38401 | gctgcaacta caggcacccg ccaccatgcc tgcctaattt tttgtatttt ttagtggaga |
| 38461 | tggggtttca ctgtgttagc caggatggtc tctatctcct gacctcgtga tcgacccgcc |
| 38521 | tcagactccc aaagtgctgg gattacaggc atgagccacc acgcctggcc aacaccccca |
| 38581 | ttttgtatac cacactcaac tcccccattt tctacatgct gatcaaaacc aatctgaatg |
| 38641 | gagtctaggt ctggctgtgg gctgttggga aSaggccccc aaatctggcc atcaactggc |
| 38701 | cgcaaaactg gccataaaca aaatctctgc tgcactgtga catgctcatg atggccatga |
| 38761 | ctcccacact ggaatgaggg caaggaacac ctggcccacc cagggcggaa aaccgcttaa |
| 38821 | aggagttcct gaaccacaaa caatagcatg agcgatctgt gccttaagga cgtgctcctg |
| 38881 | ctgcagataa ctagccagac ccatcccttt atttcccgta agaagtgctt ttaatctata |
| 38941 | atctatagaa acaatgctta tcactggctt gctgtcaata aatatgtggg taaatctctg |
| 39001 | ttcgaggctt tcagcttcga aggctgtgag acccctgatt tcccactcca caccctatat |
| 39061 | ttctgtgtgt gtttctttaa ttcctctagc gccgctgggt tagggtctcc acgaccgagc |
| 39121 | tggtcccgca gtgggcagtg gtttgggccc gaccgagctg gtcccgcagt gggcggtggt |
| 39181 | ttgggcccga ccgagctggt cccgcagtgg gcggtggttt gggcccgacc gagctggtct |
| 39241 | cgcagtgggc ggtggtttgg gcccgaccga gctggtctcg cagtggtttg ggccctgctt |
| 39301 | gctcgactgg atttcaacag tgaagccaga ttagaggaca tgctggctgc cccgatcctg |
| 39361 | gacctggacc aatggtgctt cctaaacatt ccatgtcaac ttttcttttt ccaccttcca |
| 39421 | gggctgggct gtgaaagaga ttggagaggc aggtgcctgt gagagaagct tctaccagcc |
| 39481 | tgggaagtca ggtcttccat gagtgagggg aggtatctac tttgccaaac cttgttagRg |
| 39541 | aatgctgaca tagctgcctc ttcctgccca gaagctaagc ttgaatgcca cccctcacag |
| 39601 | agcttctctc ctgtctttca ctcactcctg cttatttctg tgacagaact tgacagcatg |
| 39661 | gacattgcca cttgcttcct tcagtctcct ggagtcccca tgtcctgaac acagaggagg |
| 39721 | cacccaaatg cttgctgaag ccacatccct ggaccaagtg acctgaccct cacctggagg |
| 39781 | accttcacct tcacctctca cctggaggac cctctcacct ggaagactct ctcacctgga |
| 39841 | ggaccRcccc acctgaagga ctcccaactg gaggaccctc ttacctgggg gaccccctcc |
| 39901 | gctggaggac cctctcacct ggaggaccct cttacctgga gggcccttca cctgggggac |
| 39961 | tcccccacct agaggatcct cttacctgga ggtcccctca cctggggaac ccccccccca |
| 40021 | cctagaggat cctcttacct ggagggccct tcacctgggg aacccccccc ccccacctag |
| 40081 | aggatcctct tacctggagg accccccacc gagaggaccc tcccccccca cctggaggac |
| 40141 | ctccccacat gaaggactcc cacctggagg accttcttac ctgggggaac ccgtctcacc |
| 40201 | tggaagacct tcacctgggt aatcgccgtg gcctcccact acggcgcagc cgggtcggct |
| 40261 | gcccgggctt caccctaaaa taaggcccga actgcagcag tcggagccga tccctggagc |
| 40321 | ccagcgccta gggcggaccc gcgggagcgt ctcttggtaa ccgttgctaa ggagaSgaag |
| 40381 | cccgggaagg agctttcgcg cctgcgccgc ggggccgtcc gcgtctgcgc ctgcgcgcaa |
| 40441 | gagaggcggg gccagcgctc ggcatggcgg agccagatct ggagtgcgag cagatccgtc |
| 40501 | tgaagtgtat tcgtaaggag ggcttcttca cggtgcctcc ggaacacagg gtgcgcgggg |
| 40561 | tgccacccgg gcagctctgc ccgcctcgct agcggcactg cccggctggg tctggggagc |
| 40621 | ctcgtgtcgc gctgccgcgc cgaggcttcc ggcacgggcg ggaacgacag tcccagagtt |
| 40681 | ccccgcggcg ggggcggaag ccggggcggg gcgggctcag gacccccgac agccggtccc |
| 40741 | tggagatctg aggggccggg gcgggctcag ggatgcctcg Mgcccgcgga gagacgggcc |
| 40801 | cgggacttgg gaagagcaag ctccgggatc cagctcgggc gctgctgggt tcagcgcccg |
| 40861 | agctgggctt tgcaggctga gccgcgagcc acttgtttgt ggggagaatt tacaYccgcg |
| 40921 | acgagttcga gcttgaaggc tcccgctggg cttgggctgc atggagcggg gtccagcgct |
| 40981 | gcctggcggc ttcacgcgaa ccctgggcgc gtcccttgcg gaggtgccgg tgcctccctc |
| 41041 | tgcagagacc aggagagagc ctctgggcgt tgggcggtgt tgcccaggtg cacggctggt |
| 41101 | gggggtggag agccctgaac tttggccgct ggtttgtgtt tttaactgct ggctggtgct |
| 41161 | ctgagaggcc aagccacgtg ttcagtaaga atcattaaca gatcgtggct ggatgacaac |
| 41221 | aggagecaca ctttactcat ccagacgtgc agcaggcgct caccgcgttg agagccgtgt |
| 41281 | gctggactct ggggtgaagt agtgagttgg ccttttcttt tttgttgttt ttttgagaca |
| 41341 | gggtctagcg ctctcgccca ggctggaatg cagtggcgtg atcacggctc agtgcatcgt |
| 41401 | cagcctcctg ggctcaagtg atcttcctgc ctcggcctcc caagtgctgg aattacaggc |
| 41461 | gtgagccacc gcccccagcc tggcctggca tttctttgag ttcaggaagt gtgacaagga |
| 41521 | tttggacacc cagaaataag cgtgtcgaga agagcacaag cagaggatgt gagaagggga |
| 41581 | attaaggcgg gaaagggagK ttgagactag gccatcagtg agtgacaggc cagatgcttc |
| 41641 | tgactcggtc cccaggtgtc aggaaggaat cagactgagg acaaatgcgg ccagtggctc |
| 41701 | acacctgtaa tctcagcaat ctgggaggct gaggtgggag gattgcttga gcccaggttt |
| 41761 | gatactgagg gttaagagaa gggaagccgg gaaatggggg ctgtgaggtt tcaaaccttg |
| 41821 | ctacgtgtgc tttttttttt tttttttttt ttgagacgga gtctcattct gtcacccagg |
| 41881 | ctggagtgca gtggcgcagt ctcggctcac tgcaagctcc acctcctggg ttcacgccat |
| 41941 | cctcctgcct cagcctccca agcagctggg actacaggcg cccgccacca cgcccagcta |
| 42001 | attttttcat atttttagta gagacggagt ttcaccgtSt tagtcagggt gctctagatc |
| 42061 | tcctgacttc gtgatccacc cacctcagcc tcctaaagtg ctgggattac aggcgtgagc |
| 42121 | cactgcgccc ggcttacatg tgcttcttag atccatggag aaggatacag actctactag |
| 42181 | ttcagagacc ccaaaataca gggcctcaag atgatgcctt ttttcactct cccctaacat |
| 42241 | tctggagaga agcagttgtt ttgcacaggt gggtcaggca ttctggttcc attttttctt |
| 42301 | cccgtcactg aggtgagaag aggtgaaagc ttcctttcac ctggaagtca cacggtccct |
| 42361 | tcagctcaca ttccaaggcc agatgccact gggtagcgtt tcataacttc caggaggtag |
| 42421 | tgatacaggg atgctgggac agcagcctct gccacagatg tatttttgta tgtgggattg |
| 42481 | aaagggagta tctgagtggc cgcctctgat atttctttca aagcattggc ccagtatcgt |
| 42541 | caccaccttt tctcatcaga Raagcccaag aggccgggta cggtggctca cgcttgtaat |
| 42601 | cccagcactt tgggaggcca aggcgggcag atcacttgag gtcaggagct caagaccagc |
| 42661 | ctggccaaca tggtgaaacc cagtctctac taaaaataga aaattaggcg gttgtggtgg |
| 42721 | cacctgcctg taatcccagc tactcaggag gctgaggccg gagaatcact tgaacccagg |
| 42781 | aggcggaggt tgcagtgaac catgttcttg ccactgcact ccagcctggg caacagagcg |
| 42841 | agactcagtc tcaaaaaaag aaaaaaaaag cccaaacgtg tggttgcctt tctgtttggt |
| 42901 | gagcaagacg tgagtgggct ccctggtaca aattccattt gaaatttcct cagttgttcc |
| 42961 | atcagctgcc aaatttccac actggcaaca cccttctgtt tcagctggga cgatgccgga |
| 43021 | gtgtgaagga gtttgagaag ctgaaccgca ttggagaggg tacctacggc attgtgtgtg |
| 43081 | agtggccaag gctaggacat gtggccgcag ctcgtggctg tgacagtgtg ggacgagact |
| 43141 | gtcgaagcag cagccgcgtt ggggctggaa gggactcaga ccctgtgctc tgaaacaggg |
| 43201 | cagaaacctg ttcaggaact tcaattcctg atgtagttat cacaaacRtt gtaccatttc |
| 43261 | tgggtaaacg taagcatccg gttgctaatg cccataatcc aaacgtgtca ctgcagagta |
| 43321 | cacagaactg ttagagaaat aaaatacaga actttccaga agcagcagga gctggacatg |
| 43381 | gtgctcatgt ctatcacctc agcaactctg gaggagtcac ctgagcccag gtgttttttc |
| 43441 | tttttgtttg tttttgagat gggtcttgct ctattgccca gactggagtg cagtggcatg |
| 43501 | atcatagctc actgcggcct cgacctccta ggctcaagtg atcctcccac ctcagcctcc |
| 43561 | tgagtatctg ggaccacaag agtacataag cacacccagc taatttttaa atttttggta |
| 43621 | gagatgggat ctccctatgt tgcccaggct ggtcttgaac ttctgggctc aaatgatcct |
| 43681 | cctgcctcgg cctcccaaag cactgggatt acaggtgtga gccactgcac ccggccaagc |
| 43741 | ctaggagttt gaggctgcag tgagctgtga tcacgccact gcaccccagc cttggcaaca |
| 43801 | gagtgagacc ctgtctcRaa aaaaccaaaa acaccacaca caactaaagg ttataacaga |
| 43861 | tgtgaaattg gcgtaggtca ttctggaagc tgcacacatt agcaggatgt tttgtagagc |
| 43921 | catcttcaca caaaataagg tttgcttttt tctgcataga gtaccttatt tgcttttagg |
| 43981 | acattacaag agagcacaaa gcagtgatct gcatgctaag gaggacatgg agagcctgga |
| 44041 | gccacagaac tgagtggctg gaggccaggg tccagcacag agcaaagttg ggcaccccct |
| 44101 | gcccagttca aaKgaggaaa ccctcNgggg gaacgcgtct cgggctaggg gcgttgcaca |
| 44161 | gagtggggac tgagtgtcac tgggcatgag gttgtcagag gaaagaacgg ggacccctgt |
| 44221 | ggctcaggga gagcctcccg ttcagcgcta gggagcccac gaggggcatc gagatgatgt |
| 44281 | catcaccaat gtgtttccat tccagatcgg gcccgggaca eccagacaga tgagattgtc |
| 44341 | gcactgaaga aggtgcggat ggacaaggag aaggatggtg agcaggaaat tggggtgttg |
| 44401 | ggacctcgca ctgggaggag gcagaaggat gtgagttacc tgaagtttcc tcagagcgac |
| 44461 | tgcacggtgc ttgtagcggg ggccggcctc tgggagggtt ctggtgtggc ccagcacatc |
| 44521 | acccccaggt ccacacgcag cctYagctgt ctgccaggag ggctctgcag tctccatccc |
| 44581 | ctcctccccc tcctgtctgg tgaggccacc tcactgcagg ctcagctctg ctgtcccctc |
| 44641 | cacccccagc acctgcctgt gcaggcaggg ccctgccatg ccccacatag atgtccacgg |
| 44701 | tgtttagaga ggtgccgatt cgtatttggt caaggaagag ttaaaagtgc ttattgggcc |
| 44761 | gggcatggtg gctcaYgcct gtaatcccag cactttggga ggccgaggca ggcggatcac |
| 44821 | ctgaggtcgg cagttcaaga ccagcctgac caacatggag aaaccccatc tctactaaaa |
| 44881 | atacaaaatt agccaggcgt ggtggcacat gcctgtaatc ccagctacta gggaggctga |
| 44941 | ggcaggagaa ttgcttgaac ccgggaggca gaggttgcgg tgagccgaga tggtgccatt |
| 45001 | gcactccagc ctgggcaacg agcgaaactc cgtctcaaaa aaaaaaaaat gcttattggg |
| 45061 | gtcgccccaR ttctccctga ggtggggaca ctgcagcacc tgctatcagg tgttcgtgaa |
| 45121 | gcccaagagt ggctggggtt ggggcttccc cgccatcact ggggtggggc tcgctgaggc |
| 45181 | cacctccctc cccaggcatc cccatcagca gcttgcggga gatcacgctg ctgctccgcc |
| 45241 | tgcgtcatcc gaacatcgtg gagctgaagg aggtggttgt ggggaaccac ctggagaggt |
| 45301 | acgtggtctc ctggtctgca cattgggccc tagggagcat gtgtcttggg ctagaggtgt |
| 45361 | tgcacagagc gaggactgag tgtcactggg catgaggttg tcagagaaga gatgacccca |
| 45421 | cctcaccgcc tggctcagcc cattgtctga gggggacaca ggttgtcctg cccatgtccc |
| 45481 | ctcctgcagc aggggaggct ccacttccac cccttaggag aaggccggag agtggcatgc |
| 45541 | atcttctgtt tcttccagca tcttcctggt gatgggttac tgtgagcagg acctggccag |
| 45601 | cctcctggag aatatgccaa cacccttctc ggaggctcag gtgcgtggca gaggggcctg |
| 45661 | gggtggggga atgggcttca tgggcccttg tggtgcacat ttataacgaa acagggcact |
| 45721 | tggggacttg aagagctgct gctgcctctg cctccacctc ccagtgtttc agcgagcttc |
| 45781 | agtgaggtgg aattcctgtg gtgggggcat ctgctgggag cgggcagccc cagggccgag |
| 45841 | agccttatga gggcactggt ttcctgggct gttgggagca ggcagccccg gggctgagag |
| 45901 | ccttctgagg gcactggttt cctgggctgc tgggagcggg gagccctggg cggagagcct |
| 45961 | tctgagggca ctggttttct gggctgctgg gagagtgcag ccccggggcc aagagccttg |
| 46021 | tgagggcact ggtttcctgg gctgctggga gcgtgcagcc ccggggccga gagccttctg |
| 46081 | agggcactgg tttcctgggc tattgggact gctagtcctg ctggccagtg tgggtgtggc |
| 46141 | agggcccagc tgggcttccc tgcaggctca ccctgactgg tacctctgac cctctgcaca |
| 46201 | ggtcaagtgc atcgtgctgc aggtgctccg gggcctccag tatctgcaca ggaactteat |
| 46261 | tatccacagg tgggtgacag ctaggcagag ttggaagcac aaattcggct gaggcctRac |
| 46321 | tctgctgccc ctgtggagtt actaaaagct caggggttcc cagctgcagc agcctgcccg |
| 46381 | ctgctcgcac tccatttgtg tttttttcta aaatagagaa ggggtctctc tgtgttgccc |
| 46441 | aggctggtct cacacacttg ggctcaagtg atccacccac cttggcctcc caaagtgctg |
| 46501 | ggattacagg tgggagccac cacacccagg catgcactcc gatttttaaa aggtccaaac |
| 46561 | atcaacagca tgtaagagtt aatgaaaaaa aaaataataa agatactacg ccgggcacag |
| 46621 | tggctcacgc ctgtgatccc agcactttgg gaggccgagg caggcggatc acgaggtcag |
| 46681 | gagatcgaga ccatccttgt taacacagtg aaaccccatc tctactaaaa atacaaaaaa |
| 46741 | attagccggg cgtggtggcg ggcgcctgta gtcccagcta cttgggaggc cgaggcagga |
| 46801 | gaatggcgtg aacccgggag gcggagcttg cagtgagccg ggattgctcc actgcactcc |
| 46861 | agcctgggcg acagagtgag actctgtctc aaaaaaaaaa aaaaaagata ctaaaaagcc |
| 46921 | caaagctgag gaaacgtgca ggaagtctac gggcattggt gccgtggggg agctctcagc |
| 46981 | ccctgtggcc ctctgggagc cacctgccac tgtttttcca tcacagggac ctgaaggttt |
| 47041 | ccaacttgct catgaccgac aagggttgtg tgaagacagg tgggtgcaac ttgggccagg |
| 47101 | ccctgtccct agatggcact tggtgacaca cactcccctc tctgctgcag cggatttcgg |
| 47161 | cctggcccgg gcctatggtg tcccagtaaa gccaatgacc cccaaggtgg tcactctctg |
| 47221 | gtaagtcctt ctgaagcatg gtggcccctg gggaccaggc ctgtctggtg gaggtctcct |
| 47281 | tggggatgtc aggccgaagc tgcaactggc cttgggaatg ttaagctaca gggtctgtgc |
| 47341 | acactcagaa acgttgggtc taggacagcc tccaggacac agcagggctg tgccacaaaa |
| 47401 | tggtgagaga cgggctcagt ggcgtcaagg gcctgcctag atcagacaca cctcgccttg |
| 47461 | agagctggaa acaccgtggc cgtgaaacca tctctggagg gaaggctgct tcacggactg |
| 47521 | aaggacaggc agcttgcatg ctttccctgg ttcttcctcc tggatacttt taaccaaaca |
| 47581 | gggtcatgct tagccagggc tgaatatcag agttggtcag aatcaggtca ggaccccagt |
| 47641 | gtcgtgtact ccccatctga gaacgggttg gtagtggctg gtgtggctgt gtgccgaggg |
| 47701 | tccgtggtcc ctgcgactct caggacaccg ctgcacgtga cagctctcgc ccccggcaac |
| 47761 | cattgctgtg tgtgccacct ggatctgcag ggcctggggc agagggtggt ccgagttggg |
| 47821 | acaggtttcc aggccaScac aggctctcgg ggaggctgtg tcctgtggag gcctgggctg |
| 47881 | ggggagagga gccggctgta ttgaggtggg tgcttctgtg tgtaggtacc gagcccctga |
| 47941 | actgctgttg ggaaccacca cgcagaccac cagcatcgac atgtggtgag gagataYggt |
| 48001 | taccgctcct ggggcctcag gaagggctgg gacaggagcc gggtcacctg gttcctgagc |
| 48061 | tcagcctcag gggaggggca ggagtgcagt ggggtctttg ccagcctccc actcccaggg |
| 48121 | aggtgggcct gagcctggaa acccaggagg aggtgtgaga acttagcttg ctgttctcag |
| 48181 | gctgggagca cagaggtctg gaggaggcac gcctgcccct gcacttgtca ccccgggagg |
| 48241 | cctgcggggc ccaggagggg agccagtggt ccctgcctgt ccttcacagt gtccctgacc |
| 48301 | cagSgtgcct cacactggca gggtcagcaa aggtctggct gcagtcaggt cctctgttcc |
| 48361 | tcgcgctggc ggggtcagca gacgtctggc cgcagtgagg tccactgttc tctgcagggc |
| 48421 | tgtgggctgc atactggccg agctgctggc gcacaggcct cttctccccg gcacttccga |
| 48481 | gatccaccag atcgacttga tcgtgcagct gctgggcacg cccagtgaga acatctggcc |
| 48541 | ggtgggcgtc ctgggcagac ccgcagcccc cgcccgtgcc cacgccctct gcgcccgcag |
| 48601 | cccccgcccg tgcccacgcc ctctgcgccc gcagcccccg cccgtgccca cgccctctgc |
| 48661 | gcctcagctc ctgcctccca tagggctttt ccaagctgcc actggtcggc cagtacagcc |
| 48721 | tccggaagca gccctacaac aacctgaagc acaagttccc atggctgtcg gaggccgggc |
| 48781 | tgcgcctgct gcacttcctg ttcatgtacg accctaagaa aaggtgctga tctctgcacg |
| 48841 | gggggcaggg accctcacca cccacactgt ccagaccgtt tcccagagcc cagcctcact |
| 48901 | gcggtggaga ggcccctccc caggcacagc cgctcggagt ggccacctgg cttcctttga |
| 48961 | gcatttgaat cacaggctgc tcagctgggt gggaggtgag gaagccgcac tcacaagtcg |
| 49021 | cactaacgca ggctgcctcc tccagggcga cggccgggga ctgcctggag agctcctatt |
| 49081 | tcaaggagaa gcccctacgt gagtgtgcag ggttcctgac tcgctctgtg gggtatggct |
| 49141 | gggagcccgt tttgcccggg gtgggtggtg gagaagtggc ctgctgccct cactgacggc |
| 49201 | acaccctttc tggggtaaga ggacaggggc atgtggaggc acagacagcc cagggccagg |
| 49261 | tccagaggca gagctggact cagacctggg cctgctcctc ctatctgggg ccctgccccg |
| 49321 | cccagcactg aacccttctc cctgcagcct gtgagccgga gctcatgccg acctttcccc |
| 49381 | accaccgcaa caagcgggcc gccccagcca cctccgaggg ccagagcaag cgctgtaaac |
| 49441 | cctgacggtg ggcctggcac acgcctgtat tcccacacca ggtcttccga tcagtggtgt |
| 49501 | ctgtgaaggg tgccgcgagc caggctgacc aggcgcccgg gatccagctc atccccttgg |
| 49561 | ctgggaacat cctccactga cttcctccca ctgtctgccc tgaacccact gctgccccca |
| 49621 | gaaaaaggcc gggtgacacc ggggggctcc cagcccgtgc accctggaag ggcaggtctg |
| 49681 | gcggctccat ccgtggctgc aggggtctca tgtggtcctc ctcgctatgt tggaaatgtg |
| 49741 | caaccactgc ttctYgggag gagtggtggg tgcagtcccc ccgctgtctt tgagttgtgg |
| 49801 | tggacgctgg cctgggatga gagggcccag aagaccttcg tatcccctct cagtcgcccg |
| 49861 | gggctgtccc gtgcatgggt tggctgtggg gaccccaggt gggcctggca ggactccaga |
| 49921 | tgaggacaag agggacaagg tatggggtgg gagccacaat tgaggatacc ccgagactac |
| 49981 | caggagagcc cYgggctgga ggctgagctg catccctgct ccccacatgg aggacccaac |
| 50041 | aggaggccgt ggctctgatg ctgagcgaag ctataggctc ttgttggata aaagcttttt |
| 50101 | taacagacat ggtttcacca gcgtttaatg tgctctgatg ttgaccgtcc ctctgagtgt |
| 50161 | tctggggagg agggggtggg gcggagggtc aggaaagcag gctcagcttc cagggtcagg |
| 50221 | gagttgtggg cccagagggg ctgtcacagt ggatgcaccY tgccctctcc ctcgccagac |
| 50281 | ccgagggtag ggcagaggca cctcctcgcc agcctgtggg ctgcacccac agggaatgga |
| 50341 | ggggaggggc accattacca ctggacccac caagaccccg ccgcctgggc gggctgtcca |
| 50401 | caggcccttc cgcccagcag cagtgacgct cagggccttc ccagctgtca gccactgccc |
| 50461 | gttcctgcgc ctctcgtgca cctccacatc tggtttgagg tcaggtcatt tcctgtctgt |
| 50521 | gggcccagct gctgtgacac ccaaggggag ggccggcgYc cccgaagcca ggtcagccgt |
| 50581 | gcacccggca gagccccgca gattggcYcc agcagagctt ggggtggggg gagctcggtg |
| 50641 | tcaccaacag gcccttgagg acactcgtgt ggagaatccc tgggacacgt ggaggacccc |
| 50701 | caaStcctga gccccgtact ccgtactgca gggagcaggc caggagccac gggccttggg |
| 50761 | gcacagggtc cttctcaggg acaggttcag gcactggctg gaacaggctg gacccctcta |
| 50821 | cccagcacat gtgggcatgc gtctgggttc cgagggtggg gaaatgtgga aaggctcctg |
| 50881 | ctggccggct taggcctgct gcccttggag ccttccatat acagagagtc ccacctccag |
| 50941 | caagggttgg cctggactct ccaWccccct gctgtgccca ggactccccc agggacaagg |
| 51001 | caaccagagg cccagacccc tccccagcaa agagaagcac cacgggagct gtctcccaag |
| 51061 | agcccttgac ctggggccca gctggcctag ggccgggccc cggggctgtt gtagagcaga |
| 51121 | gtgtccatct gtgcacgctg tagccacaag cgccgctggg cgtcgccaag cagcacacgc |
| 51181 | agcgagtggc ctggacggca ggcgggcagg gctgcacagt gggcagcatg cagctggcga |
| 51241 | caggtgtcgc agcagagggt gggcagggcg ccaggggaca ggcagctgtg tgcctggtag |
| 51301 | cctgggggct ccgagacaga cctataggct gaggctggta ccccctctgc ccgaatacgc |
| 51361 | ctcgggctgg ccgctgcaga gctttcaggg gtggccaggt ccccaggcct actcagctca |
| 51421 | cggcgcagag agaggaagga gaaggcggaa ggttcaggtt ccagcccctc ctccaaggcc |
| 51481 | ccatatggtg ggctgctggc ctgcggcagc tcctcagctg ggggctccca ggcccggccc |
| 51541 | ccagtgcccc acagtttggc actctgctcc cagagtggtg gcctgtaggc cagctccgcc |
| 51601 | gggggcgagt cagggcctgg tgacggctcc ccatacaggc tggcgtcctc cgacccctcg |
| 51661 | tcttcctgca agtcccggta taagtccagt ggggccctgt aagcagcagg ggagcctcgg |
| 51721 | gggggcaggg gtggcggctc ctcatcctgg gccagccgct gctgcagcca ggccacacag |
| 51781 | gaggccacgt ccccgctggc acgccgtgcc tgcagcagct cctcagctgg cagcacactg |
| 51841 | gtgcccagct gggccagcac ctcacccagg atctcacact ccagccggag ggcgaagcag |
| 51901 | cccagggcca cctgcaccag ctggcaggcg gggggcaggg cggtcaccat gagccgatgg |
| 51961 | ctgtctctgc gtacgtagcc catcttctgg aagctcttca ggaggaggtc gtctgagagc |
| 52021 | acacccttca gcacgtgcac gtagccccca gagaaggtct gcaagggagc ggccaggtca |
| 52081 | gtgactgccc accctcccat aggccagacc ctagggtaca gtgggtccgg gatctgttgg |
| 52141 | ctcaggcctt ggcagcttct gatgctcaga cccaacccca gagactaggc ttagccagct |
| 52201 | tgggccaggc ccaaacatga tgatttcttt tttttttttt tttttttttt ttttttgaga |
| 52261 | cagagtcttg ctctgtcatc caggctggag tgcagtggct ccatctcagc tcactgcagc |
| 52321 | ctctgcgtcc caggttcaag caagttcccc tgcctcagcc tcccgggtag ctgggactac |
| 52381 | aggcgcccac caccacaccc ggctaatttt catattttta gtagagacag ggtttcacca |
| 52441 | tgttggccat gctggtctca aactcctgac ctcaagtgat ccgcctgcct tgagctccca |
| 52501 | aagtgctggg attacaagcg tgagccactg tgcccggcct aRaatttttt tttttttttc |
| 52561 | ttttttgaga cagagtctcg ctctgtctgg agtacagtgg cacgatctcg gctcactaca |
| 52621 | acctcctcct cccgggttca agcgatcttc ctgcctcagc ctcctgagta gctgggatta |
| 52681 | caggtgcgca ccaccacgcc ccactaattt ttgtatattt agtagagacg gggtttcacc |
| 52741 | atgttgacca ggaaggtctt gatcccttga cctcatgatc cacccacctt ggcctcccga |
| 52801 | agtgctggga ttacaggtgt gagccaccgc acctggccga atttcttaaa tttaatggaa |
| 52861 | aaaacaatga atacattaac ctagctcaca aatcagaaca aaatgaagag cccctgtact |
| 52921 | gagaaattcc actgtcttct gtcccaccgc tcccttcacc cctggagatg gcctcatcta |
| 52981 | attagttttg aatgtacttg cccaggtgtt tgttttgcaa acccaaatac agatttcgtt |
| 53041 | tgctcctcgt atgaggtagt cccccctttt ttttcacctc tttgtaaggt ctctaaccaa |
| 53101 | agctagacag gtagcctctt aaacgtgttg ttctgaactt tgtttttttc cactacagtc |
| 53161 | ttgcagacaa ggacagaagc tgtaacgttc attccccctg ctgtgtagta ttccaccatg |
| 53221 | ggggtgacac gtctgttttc agcgctctcc agaggctgtc catgcactgg gctgacagcc |
| 53281 | gtgggacggt agaagggtga gtgctcNttt tttttttttt ttgagacgga gtctcactct |
| 53341 | gtcaccagga tggagtgcag tggctcgatc tctgctcact acaacctcca cctcccaggt |
| 53401 | tcaagtgatt ctcctccctc agcctcctga gtagctggga ttacaggcgc gtgccaccac |
| 53461 | acccagctca tttttgtatt ttttaataga gaYggggttt caccatgttg gttaggctgg |
| 53521 | tgtcaatctc ctgacctcgt gatctgccca cctcggcctc ccaaagagct gggattacag |
| 53581 | gcgtgagcca ccgcgcccgg cctggctgag tgctttctag cctacactta ggtgaagaac |
| 53641 | taaaggagct caggacaatc actgcgagcg tctcctgact ctcaaattcc ctaactccgg |
| 53701 | gactcctagc atctaatgcc tggacacggc ccacgtgtgg agcgcccagt gcctgcagca |
| 53761 | ctgagctcgg ggctttgatt cattcctctg gccccatgaa ctggactgtt cccgtgtcgc |
| 53821 | tgtctcagga ggaaaggggc tgacagagat tagctaattt ggccaagtcc tcgcggccag |
| 53881 | gaagcagcag aactaagtcc aaaccagggc tgtccSgcac tgaagccggt tattcccccg |
| 53941 | ccccgccccc tgggaggcct caagggcggg cctgtccgtc ctctctcatg aatgaaagtc |
| 54001 | ctggcgcctc cgaccagcag gtgtccccgg ggagccgcgg agagactgcc gctgccatgg |
| 54061 | caaccggcgg aggcgtagct cgcccacgtg cccagtgacg tcatcggctt cctactcttc |
| 54121 | cgaattagct cagagctccc ggcggccctg ccccctcctg ccgccaggag ctcacctgga |
| 54181 | gaggccaggc cagccctggg cacagcctcc cgccccaggc cgtggagttc actaggtctg |
| 54241 | agcccagccc tgagacttca cagggatgca cccatctgca gtcctcccag cctcggcggc |
| 54301 | ctcctctgca aatgggattg gcccataacc tggaggaatt aggatgacca tgggaactca |
| 54361 | agtcccttct gtgcccccgt cctaagattt ccatggcaac tggggccgcg aggcctctta |
| 54421 | gggaagacac tgacgcaagc tctcagggca ggaccccgta ggcagcaccc aagcctgccc |
| 54481 | tcgcccaccc catgttctca gccctaggag tgggtgcccc cacacgccca cgacccctca |
| 54541 | gcgctcggag ccttgaggag cccaccaggc actgaggaca cttgaagccc ctctctctcg |
| 54601 | aaaggcgtgg tcccccatgg tcggcggccc cgcgacccgc aggccaggac gaggggcgcg |
| 54661 | ctcctcctgg cctggcgcct accttgatgg tggtgaactc cttcctccag ggcagcaggt |
| 54721 | acaggtgcac cgcggcgagc tccagaagct cgaaggcgcg agccaggccg cgtagcgcgg |
| 54781 | gcgccaggtc ggcgcggccc cacagcccgt cggtgagcag agccagcgcg tcgtcctgca |
| 54841 | gcgccccgtg caggtcgaag tcctccacca ggatctgcca gagcaccgcg cgcagcgagg |
| 54901 | ggtccccgca cacgcccgcg cggccccgtc gcagctcgcg ctccaggcac tggcggtagt |
| 54961 | cctcggacag cgagctgctg cccatcctgc ggcggacggg ggtcgggggg gtcagggacc |
| 55021 | taaggccgcg cccagcccgc cgcgctctcc ccgaaccctc acccagcgtc cccaggaccc |
| 55081 | ctcgagcctg gagaccccgc ccccagcggc tgggcgcccc cggtatcttc ggcaccccct |
| 55141 | acgctggaga cctcggcccc atcagctgcc cgcccaccca gcgatccagg cccctccagt |
| 55201 | tgagcgcccc cgcacctcca gcccctccct ccagcctggg tacctcggcc ttccaggggc |
| 55261 | gcccccggtt atcgaccgca cccttctcta gagacgcgct tccgcccgct gagccctctc |
| 55321 | ccgaccagag ccccttcgcc cggcagatgg agctgcggat cccgcgggtg acggcgctac |
| 55381 | ctgtctgtcc ccagcggtct ccggccgccg cggatcagtt cgggcctccc cgcgcgccgc |
| 55441 | gcgcttccgc cgggagtcgg ctccgcccgg ggtccgcggc cgtgacgctg ggggcggggc |
| 55501 | ggggcttggc gcaggccccg cctccgaata atttaggagg ggcggggcct ggtgaaaggg |
| 55561 | gaggaggagg tgtcagcccg gccgagcccc tgcgcgaggc accccgagag cctcctgtca |
| 55621 | ccgcgtcgcc cgtctcctcg cgggaccggt atcgttcacg acgccgctgg gccgagggct |
| 55681 | gcgggctcat ggcaggcgct cagggaacgt tgtgcctggg ccgctcctcc ccacgagaca |
| 55741 | gggacccagc cccaggcgac ccggccgcgg ggaagcaggt gcgcgggctc gtcccctggg |
| 55801 | gggtggggcc accggcatta cctggcgggg attggctgga tcctgggaag agcctttcct |
| 55861 | gcggggcggg gccacctgca ttacctggcg gggattggcc ggatccgggg gcgggcctcc |
| 55921 | cctgtggggc ggggccatcc ccttgaggga ggggcccgcg gcacggaagg tgggcggaag |
| 55981 | gacaagcctt tcagcctcgt ctgaaacagc agcctcaggt ggatgcgggg acctcagatt |
| 56041 | ctgtttatgc tggctccgct tcccggcctc ctgcacttgc catctgcagc ccttgagagc |
| 56101 | atcgtctggg cgggtctctg aggcagggcg aagccgcccc cctctctagg ctaaaagctg |
| 56161 | acctgggctg cggatccacc caggctaaat ctgggccacg acttcccggg gacgttttct |
| 56221 | tcgctcgggt tcctctgttc tgtgctacag ccactttctc gcattgccca gaagtagagg |
| 56281 | aggatgaggg agacccttgg aggtcccagc cgaaacccac ccctacccca cacccccgct |
| 56341 | ttgggcaggc tttgctactt cacgtctgtg tccctcatct ctccaggcca ccaaaaccaa |
| 56401 | ggcccaagtt tgttgaaact ttccctgggc ctgggcggcg aggtcccccc tttttcttag |
| 56461 | attttttacc tggaagttcc ctgctggcat cttagaaata gtgatcatga ccgggtgcgg |
| 56521 | tggctcacgc ctgtaatccc agcactttga gaggccgagg cgggcggatc acctgaagtc |
| 56581 | aggagttcaa gaccagcctg gccaacatgg tgaaaccccg tctctattaa aaatacgaaa |
| 56641 | gttagccggg cgtggtggca catgtctgta agcccagcta cttgggaggc tgaggcagga |
| 56701 | gaattacttg aacctgggag gcagaggttg cggttgcagt gagctaagat cgtgccactg |
| 56761 | cactccagcc tgggtgacag agcgagaatg tctggaaaaa aaaatcgtga tgcagtcggt |
| 56821 | ggatctccct gagttatcta ggtggccacc tctggacagg gagctctttt acttactgtt |
| 56881 | tactgaactc actcttggtg cccagtgcca ccatgggtcc acaccagtcc tcaggacagc |
| 56941 | taacttcgta agtctcatct ttattcttct tctgatgcgg aggagggtgg aggtaactgt |
| 57001 | tctgcctttg tcctcactga atttggcaac accagtccat ccatccagca gtgggcctag |
| 57061 | ctctcctccc ataggcctgg gtgggttttt gcacctgcct caacccatgg aatagaactg |
| 57121 | atgtggaatg aattccaagg ctaaatcata aacatgccat accctggcct tgtcttgaga |
| 57181 | tccttggcct ctgacatggt ttggttctgt atccacaccc aaaatctcat cttgaattgt |
| 57241 | aatcccacag gtctggagca ggaccaggtg gagataatta aatcttgggg gcagcttcct |
| 57301 | ccatgctgtt ctcgtgatag tgaattccac gagatccgat ggttttataa ggggttctcc |
| 57361 | cctttgttca gtgctcattc tcgctcctgt cgccctgtga ggagacgcct tctgccatga |
| 57421 | ttgtgagttt gctgaggcct tccccagcca tgcggaactg tgagtcaatt aaacgtcttt |
| 57481 | tctttataaa ttatccagcc tcaggcagct atttacagca gcatggggac ggactaacac |
| 57541 | accctcagaa cccagccact agagtgagaa ggccacggcc gtgtgcagag cttttggcct |
| 57601 | gcaacttcga cctgcgactc acattcacct gcagcctatt cttgggactt ttttggaaat |
| 57661 | agtttttcca gacaaaaatc aagatgagtt tgttctgggc cgagcgcagt ggctcacgcc |
| 57721 | tgtaatccca gcactctgga agaccgaggc aggtggatca cgaactcagg agatcgagac |
| 57781 | catcctggct aacacactga aaccccatct ctactaaaaa atacaaaaaa ttagctgggc |
| 57841 | gtggtggcgg gcatctgtag tcccagctac tcgggagact gaggcaggag aatggcgtca |
| 57901 | acccaggagg cggagcttgc agtgagccgg gatcgcgcca ctgcactcca gcctgggcca |
| 57961 | cagagcaaga ctctatctca aaaaaaaaaa aaaaaaaagg atgaggttgt tctgggttgg |
| 58021 | catgggccct caatccagta tgactgctgt ccttatgagg agagagaaac ttagatatga |
| 58081 | acgcacagga gagaagaccg cttaaagacg gaggcagagt ggaacgaggg tgccgtgagc |
| 58141 | gaaggtaaca cagctgtgtt agagccacca gaaactggag gcaaacagaa ttctacccta |
| 58201 | aagcctccag agggagtcag ccctgctgac agcttgattt tgacttcagg cctgcaagat |
| 58261 | ggtgagagag aacatttctg ttgctgtaag ccacccattt tgtggtcgtt ttggcagccc |
| 58321 | ctggcaacac atccagtcct cacgtcgtcg ttccgacagc cctgaatgag gtcccagccg |
| 58381 | acagctggca ccaaccatag acagcagaca gctcttcgga tgaatccaac ccctggtcat |
| 58441 | tgggttacca cctagaaaca gcctcccatg atgctgagga gccatctggc tgatttttgt |
| 58501 | actttttgta ttttcagcag agacagggct tcactatgtt ggccaggctg gtctcaaact |
| 58561 | cctgacctca ggtgatccac ctacctcggc ctcccacagt gctgggattg caggcgtgag |
| 58621 | ccacggtgcc cagcctaatt tttaactttt tagtagagag gggggcctta ctatgttgcc |
| 58681 | caggctggtc tcaaactcct gggctcaagt gatccccccg cctcagcctt ccgaagtgct |
| 58741 | gggattccag gtgtgaacca ccatgcctgg cgtcttgagt atttaagccc ctgagttggg |
| 58801 | gtagtttgtt aggcagcggt ggctacgtgg gctgtacaca ttcacggctc cctcccttcc |
| 58861 | tgcccgggga tggacactcc atgagctgcc gtgtggattc tccgtccttc cctgccctcc |
| 58921 | cgggacagac actccattgc ctccactgca tatacatcct ctaaaaccgg cagcagcctt |
| 58981 | gccaggtcgg aggttccgtc cacacttacc tggactccag gactgcacct ggtcccctgg |
| 59041 | ggccactgcc gtgtccaccc cacagtgtgc gtgagctccc tccccctcct gtactcagca |
| 59101 | tcgtccagct tcctcatctt ctccggtcca atggatataa ggttacatct tgtttacttt |
| 59161 | tcatttgatt tttgttatga tttggacact gcttatgcag aatagtttta tttttatttt |
| 59221 | tttttagatg gagtctcgct ctgtgctcag gctggagtac aatggcgtga tctcggctca |
| 59281 | ctgcagcctc tgcctctcag gttcaaacaa ttctccggcc tcagcctcct gagtaactgg |
| 59341 | gactaccgcc accacgctca gctaatttat gtatttttag tagagacggg gtttcgccat |
| 59401 | gttggccggg ctggtctcga actcctgacc tcaaaagagc cgtccgcctc ggcttcccaa |
| 59461 | accgctgggg ttacaggcat gagccactgc gcccagccat tactttttat tttattttta |
| 59521 | ttttttgaga cggagtttct ctctgtcgtc caggctggag tgcagtggca cgatctcagc |
| 59581 | tcactgcaac ctctgcctct caggttcacg ccattctccc gcctcagcct cccgagtagc |
| 59641 | tgggactaca ggtgcgggcc accacgcccg gctgattttt tgtattttta gtagagacgg |
| 59701 | ggtttcaccg tgttRgccag gatggtctYg atctcctgac ctcgtgatcY gcccacctYg |
| 59761 | Rcctcccaaa gtgctgggat tacaggcgtg agccaccgcg cccggccatt tgtaggattt |
| 59821 | ctaaaaggaa gctgctatta ttcattcctg tcttatgggg gagctgcagg ctcagcgagg |
| 59881 | gcaaagctgc acagcaaaca gcagcacttg aacatgtctg ccgggtctca gcgccaggga |
| 59941 | tacagctgag ccaggacgtt cccRgccccc acagtgtgca ggctgcaggt gtggccccga |
| 60001 | tgccaagcca gcaggggctc ccacgccagg atttgttcaa gacacagctc atctccacca |
| 60061 | ttctatgaag cggactttaa aatcccatct tctagacacc aacgctgagg ccggagcagc |
| 60121 | ccaagctcca aggcaccgtg gccagctggg gttcgtatcg agggctgggg cagatgctcc |
| 60181 | agctctgccc cacccagaga gacacttctg tctccctatg gggtccttgg cgggtaaacc |
| 60241 | agcccctgct Sagggtgcaa gcacagagca tcccaccgag gtccacagct actgctttta |
| 60301 | agactgtttc cctgagaaca gcaacactgc cccccagcca gctcctggta gcatctaggg |
| 60361 | ttgggacctc ccaccccacc agccctgcca cctgcctcct catggcatct caaggtgcca |
| 60421 | ggggtgcctt tcttgcagaa gagccctcca tcctgatctg cccacaccgc cctggcagat |
| 60481 | gctgtccacg cccggcagcc ttctgtgcac ctcagagcat gtgtgtgtcg ccatgcacag |
| 60541 | aacgatgatt cctgagaccc tcgggtgact gctctgggct caggtcccca gtgatgacct |
| 60601 | ggaccccagc ctcaccccac ccttcccacc cactgatgcc ctgcagagct ctcctgtcca |
| 60661 | agagggtgaa gcccacacct gcctggccag gcacagcccc aagaggaccc tgcttctatg |
| 60721 | cagagggtct cggaggctgg cagatgttca agaggttggg ccaggcttgg tttggggctg |
| 60781 | ggctgtggct cgtcccccac atcctcagcg gcctctgcca tgggcagcag gggtggttgt |
| 60841 | gctgcctggc cagtggggtg gggattgagg gagccacacg gcgaagcaca gcagtgagct |
| 60901 | ctttatttta ggctgggtgg gggcagcatg tgacagtcaa gagggctgac ccttgggctg |
| 60961 | cacggaccta gataagatgc ccagggtccc ggaatgttca cagcccacag ccaaggtgag |
| 61021 | taaggggcag ggaatgtcct gggctcagag cccgagaccc tgcaggggtg tccctgccca |
| 61081 | tcagcaaaca gcataccctg acaccagctg caccccaggc cccagccggc ccttgttcct |
| 61141 | gacccagaca agaaactagt tcatggggct acaactcccc ttctaggggt cagaggggag |
| 61201 | agaaactgga ctgccgtctc cactgctggc cggctcatgg ccccctcttc cctgatgctc |
| 61261 | cccaggagct gccccagcca gatgttcatc gctgagcctg cacccaccga agcccaaagc |
| 61321 | ttcccttctt ggcctctctg aggcctacac aggagagcag ggcctggttc ctcctggagc |
| 61381 | tgattcagcc ccattctgtc ggggcagagg tgccaacccc aagctccagg gtcagatgtg |
| 61441 | aggtgaggaa ggtgaagagc tggagagcac accacagtgg acaagccccc accatgtgca |
| 61501 | gagcagcgtg aggctccagg atggtcctca gtagatccca tggctccagg tgtaggagag |
| 61561 | acagccctgg gaggcgaggc caggccctgc agggaccctg ggctctagct gtactacttg |
| 61621 | gccaggcctc cccggggcag cagctccacg tagctcaggg cactctgcag tgatgtgagg |
| 61681 | cagtagccct cctctccgat caggtacctg catggatggc aggggcagcc ggtgtcatct |
| 61741 | Raacgaacag aaggataccc ccggccctcc agaaggtagt gtctgccctc ccagttcagg |
| 61801 | acctgggtgc tgagggtggg caggtggggc tgtcagggcg gctgggctag aggaggcctt |
| 61861 | tcccacccac tatgtgctcg tccttggacg gtagagccag ggatggcact ggatccttat |
| 61921 | cccctcccca ggcagatgct gctcagggga cagcccccag ctcctgctgt ctgggagagg |
| 61981 | ctgagctaag cgaagggcca cgggacatgt ggcagcaggc actctgcaca ggtggaccca |
| 62041 | caggggctgc ccgaaggcat ggctgtgacg gagccacacg ctaccctccc cagctgctac |
| 62101 | tgacaaccac cgtttaggtt gagggatgag gtccctgctc ttcgcacaga gacccccacc |
| 62161 | ctgacccgcc agggtctacc cctcgtggat gaactcctcc agggccgcgc actccgacac |
| 62221 | cagctgaggg aggccgctcc tcagcaccac gaaggacagg atgggcagca ggtcatcggc |
| 62281 | accactgtcg ggagggcata gcggccttgg gttggggcca ggagcctctc actccgcaaa |
| 62341 | cccagcagct gtggagccat cctggccgtg ctggccgcca tctctgtgcc cttctgcgca |
| 62401 | gaggcaccgg aaggcagctg ggcatggtga ggctgtccct aaagggagct cccgtactgc |
| 62461 | ggagccaggg ctgggcccac acggccctcc tgcttctaca gtgcagccag ctggcacaag |
| 62521 | gctaaggggc aggaagatag tccctcccgt gaccattgcc tttctagggc ccttcctccc |
| 62581 | tggccacacc tcgccctcct gggactcaca tggcagctgc agcgatgggc gggggcccgg |
| 62641 | cctggggtgt ggcctctggg gtggggcagt agtcttccgc acagacacag atgatccgca |
| 62701 | gggtccgcac tgcgccccag acagggtttc aggggcagtt aaccccgtcc agcagcctga |
| 62761 | gccacccctt ataccccgcc cacccaccca cctcctgatg tgcacggctg ggaagagcca |
| 62821 | cggtgaccct ggcgatgttg ctcagacacc acttttgttc tggtccccca acctccccac |
| 62881 | cggcacgtga caagagaagc tcagtggtga agacacaggc tccaggtcac ctactgggat |
| 62941 | gggagcctgt ccctccccct gactctcgaa cctgctgacc tatgcactcc agcttcttct |
| 63001 | gggggcagct ctccaggacc agcagtccga gctcctgggc cgccgcgcag taggggtagc |
| 63061 | cagtggcccc cttggcctca gggttctggg ggaggagctt ggtggggatg ccaatggcgg |
| 63121 | tggggggtgc attcctgtag agctccatgc tcctgctcag ggcagcctcc cgggctcggt |
| 63181 | gcacgctcct ggggcagaga gaggccaggc taagctgccc cagggacctt gccctgggga |
| 63241 | cagaagcagg gctgtggctg gagctcagtc ctggagaaaa ctggctatgt atctcaagca |
| 63301 | aaaagcccca gcgttgccaa gaccccaggc acgggagagt ggcaataatc ctacctgcaa |
| 63361 | actggaagcc cctgaggatg gcctcagggt aaagcacagg ctagggggag caaacacgag |
| 63421 | tcttggggat ggaggggagc agacagtgcc agggagcagg gcaggcctcc tgggggactg |
| 63481 | ggctggaaga cactaggcca cgcaggtggt tgtacagctg ggtccatacc tgtacagggc |
| 63541 | cagcagcaga ggccacagcg gggagaaaaa gggttcctca atgcaggcca ggcagcggtc |
| 63601 | cttggaggca gctgtgttta ggccttcgaa ggccagaagg gtcagcgaga gcagcctgtc |
| 63661 | tgctaggaac agagccgggg acgtccacag aggctcctcc cctgctgggt caagtggctc |
| 63721 | taagccacgg ggcctttctc cgcccaagaa acctctttcc accgccttca agcctcctgc |
| 63781 | acccacccct gaccctcctc ggcccagggc agggagtccg ggactggaaa cggtcaccta |
| 63841 | cagctgctgc cctgttgggc cagcagcctg agaagaaacc ggagaatgat ccttttctca |
| 63901 | aaaggggctt tacccaaaac tgcaggaacc agggaagact ttagatccaa ccccctgtgg |
| 63961 | gtggggaccc tgaccaagtt ccttagtgtg tgggcagcac agtgagaatg acgcctggac |
| 64021 | tgttgggcct ccctcagtgg ttcttagaaa actctgggct gggcgcggtg gctcatgctt |
| 64081 | gtaatcccag cactttggga ggccggggtg ggcagatcaa ctgaggtcag gagtttgaga |
| 64141 | acagcttgac caacatggtg aaactccgtt tctaccacac tgtttccaaa atgagaaggg |
| 64201 | ctgaggcgtg agctgggtgg atgtaagtct gatcagagtc tctgatcaac agacccttcc |
| 64261 | ccaaacagaa gcttgaaacg gaccaataag caggggtgaa gagctgcggc ggctctccca |
| 64321 | gggagggcct ggcctcacct acggcattgt ggatctcctc caccgcggtc ttcagctgct |
| 64381 | gcagctgggg ctccggctgt accccacggc cccggccctg ccgctcagac gtccccagga |
| 64441 | actgctccag gtcctcgaac gagctgtcct tgtctggcaa tccagatgcg gtgtccccca |
| 64501 | ggggtgaggg agaccccacg gggccgggtt ggagtggggg cgtgggggga ctgtcctggg |
| 64561 | gccgcggggc tgcgctgggc tcgggcgggg acagcatgca atggaggctc tgcgagggcc |
| 64621 | gcagccgtcg gcttccgggg ttgggggtcg gggggcagca gcctggggct ggcgcggctg |
| 64681 | ctctgctcac ggcgggatac agggcgctgt acacggagca ctgcagccgc ctcaggagct |
| 64741 | gcgcgatcgg gtggtcgggg aggctgcggg agaagaggac gtgagaacgc ggccagcctc |
| 64801 | ggcctctccg tggcatccac aggtgccgcg ctatgcccgg tttcagagaa gcgacttgca |
| 64861 | ttacctcctg acagtgaatg ttggaaagtc tgccccccgc ccccgctggg gaggttggag |
| 64921 | aagccgagga aacgccctca gcgcaaaggg agctgcagtg tccacgtggc cgggcacagg |
| 64981 | ctccctgtgt cagtccagcc cctctccggc acctggcacc aggcagaggt gcccaaggcc |
| 65041 | ggtgcgcagg ggctctgtgg ggcctgaagg cccagctacc tgagcaggtg tgagaccagg |
| 65101 | ctggtcacga gtgacaggtc ccccggattc ctcttgagct tggccttcca gtgcttcggc |
| 65161 | cagtcctacg ggacaggggg ccttgaagga aagcacggtg ggtccgccac gaccggacgc |
| 65221 | gcctcatctc ccaccagtgc cgacccctag agactgtggg agcccgtcct ggggtgcagg |
| 65281 | agacccagcg ccagcaggcc ccgcgacccg cccccgcccc cacccacacc cgagggaaac |
| 65341 | cttaggcgaa ccctgggcct gcaccctcgc tgggaccctc cctcctcgca gggaccctcc |
| 65401 | ctcctcgctg ggaccctccc acgcgacccc tgacctcgcc tcctcgctct gccccgcccc |
| 65461 | acgcagccct ggccccgccc cctcaccggg ccctcccacg ggcctctggc cccgccccct |
| 65521 | cactgccccc cacagcctct ggctccgccc cccacggggg cccacccagg cggctctgac |
| 65581 | cccgcccccc tcgctgggct cctcctacaa agcctgggcc cgtgggggac gcacatggtc |
| 65641 | ctgttcgtac tccaggatgg cggcgtaaag ggcccgctgc tcccgttcct ccggggtcag |
| 65701 | ggcgacttgg ctgcaaaacc tcctggggag aaaggcacgc ggtgggtgcc ggggccaggc |
| 65761 | cctccccgca gcggccccag gggcgggcag gagtcccacc tgttggcggc ctcctggagc |
| 65821 | cgcagccggc gctcctccat ctttctctgg agctgggtgc agagtcaagg ggccgagcgt |
| 65881 | gggatctgag cgggccctcg gttcccaacc ccgtcccaca cccggagagg ccgggacgtc |
| 65941 | ccagggacct cctctcggtg aggggctgtc cccaggtaag gcttctgggg cagaaggcag |
| 66001 | cctgcgctca agccaggagg gccgggccag agaacatggg ggacggcggc cgggccttcc |
| 66061 | cctgagaccc tgcctctgca aagcggggag cccctcggcc ctgtaccccg accccggaac |
| 66121 | ctccgctccc ctcgctgccc ccttctctgc ccttggctca aggccacact gagtttcccc |
| 66181 | tcggggacca ccagggcggt cagaaaggct gctgtctcct cccgggcttt ggcaatcact |
| 66241 | aggttctcca tcatctgccg ctgtagagag agggtctggg gggggaggag ggagtaaggc |
| 66301 | cgactcccct ctgccccgcg cccacgcacc cccaggccct ccctagcccc cactcaccag |
| 66361 | ggatgtcttc tgcatggcct ggctggggtc tagccgcgcc attcgggcct catacgcagc |
| 66421 | cttcagcttc tgattctgca gggaggcctc ctccagtggc gtcagctctc tggaaatgtg |
| 66481 | acaagctgct gtctagaccc caggaagccc agtggtgtct ggacaccaga ggagtctctc |
| 66541 | tcatcctccc ggattgctct gaggccagga ggtgaggaga aagaggaggg gagccatctg |
| 66601 | gggtgggctg caacatctgc cccttcttgc ctgcccttcc aaaacacagg ttttggccag |
| 66661 | gtgcagtggc tcccgcctct aatgccagca ctttaggagg ctaaggccgg gcggatcacc |
| 66721 | tgaggtcagg agttcgagac cagcctggcc aacatggtga aactctgtct ctactaaaaa |
| 66781 | tacaaaaatt agctgggcat ggtgcacact tgtaatccca gctactcggg aggctgaggc |
| 66841 | agaatagctt gatcccggga ggtgatgcag tgagccgaga tctggccact gcactccagc |
| 66901 | tggggtgata gagtgagact ctgtctcaaa aaaacaaaac caaaacaaaa aataacaaaa |
| 66961 | agaaagaaaa cacaggtttt ggtattgcca ggacttttgg tttttctttt tttctttttt |
| 67021 | tttttgagac ggagtctcgc tctgtcgccc aggctggagc gcagtggcgc aatctcggct |
| 67081 | cactgcaaac tccgcctccc gggttcatgc cattctcctg cctcagcctc ctgagtagct |
| 67141 | gggactacag gtgcccgcca ctatgcccgg ctaatttttt gtatttttag tagagacggg |
| 67201 | gtttcaccgc attaaccagg atggtcttga tctcctgacc tcgtgatcca cccgcctcgg |
| 67261 | cctcccaaag tactgggatt acaggcgtga gctaccgcgc ccggccggac ttttggtttt |
| 67321 | tcaagagaag ctggaccact ggacttctat ggagatccgt tatatgtgtt tggcaacagt |
| 67381 | atgaatcttg ttaaagcaag cttgtgcaac aatccgaggc tgtatgactt cgaatgtggc |
| 67441 | ccaacacaaa ttcctaaact ttcttaaaac atcactgagg gctgggcacg gtggctcacg |
| 67501 | cctgtaatcc cagcactttg ggaggatgag gcaggcggat aatgaggtca ggagattgag |
| 67561 | accatcctgg ctaacatggt gaaaccccgt ctctattaaa aatacaaaaa attagccggg |
| 67621 | tgtggtggca tgtgcctgtg ctccagcctg ggtgatagag actctgtctc aaaaaatcac |
| 67681 | tgaggttttt ttgcaaaatt tttttttttg agtcagtctc attctgtcgc ccaggctgga |
| 67741 | gtgcagtgac acaatctcaa ctcactgcaa cctccgcctc ctgggttcaa gtgattctcc |
| 67801 | tgcctcagcc tcccgagtag ctgggattac aggcatgtga caccacgccc agctaatttt |
| 67861 | tgtatttttt agtagagatg gggtttcatc atgttggcca ggctgatctc gaactcctga |
| 67921 | cctcaggtaa tctgcccgcc ttggccttcc aaagtgctgg gattacaggc gggagccact |
| 67981 | gcgcccagcc ttgcaatttt tttttttaag ctcatcggct gttgttagtg ttagcgtatt |
| 68041 | ttatgtgtgg cccaagacaa ttcttccagt gtggcccagg gaaggcaaaa gattggacac |
| 68101 | ccctgcttta aaggcactat gtgggtcaaa cagccagatt agtctgtgac ccatgaaagg |
| 68161 | aagaagtgtc ctgtctcctt tatacagttc ccaacagcca ctccaccttg aaagctttgt |
| 68221 | tccctcgtac cttcccactg tcagggacaa tggaggacaa ggaggaaagt cagagcctga |
| 68281 | cgcctggcca ccctgccctg aagccacatt gcagaaacaa ggggactggg aagccaggcc |
| 68341 | ccgccagagc aggggcagga ggagtggacc cttctcgtct ctcccacttg ggagtcactg |
| 68401 | ggctttttgc atttgtgtct ttgtatcttt catcagttct agacaactgt ctgccattct |
| 68461 | aacttctaat aatgcctctc tcccattttc tttcttcttt ttttttgagc cggagtctcg |
| 68521 | ctctgttgct aggctggagt gcagtggcac gatcttggct cactgcaacc tccacctcct |
| 68581 | gggttcaagc gattctcctg cctcagcctc ccaagtagct ggggttacag gcacgcgcca |
| 68641 | ccatgcccag ctaagttttg tatttttagt agagatgggg tttcaccatg ttagccaggc |
| 68701 | tggtcttgaa ctcctgacct tgtgatctgc ctgccttggc ctccctccca ttttctttct |
| 68761 | ggaactccaa cgaacacgtt aggctttcca tctcttgttt tctgtcttta ttttttgttt |
| 68821 | ctgtaattca ttctagctat cttttgacct tttttccaat acatgaattt gctcttcagc |
| 68881 | tatatctgct ctgctgttat aaacttgtct gctcagtttt gtatgtatgg atttctacat |
| 68941 | atatatagat atataactca tccttgttcc cagctaaatt tttttttttt tttttttgag |
| 69001 | atggagtctt gctctgtcgc ccaggctgga atgcagtggc gcaatctcaa ctcactgcaa |
| 69061 | cctctgcctc ctgggttcaa gggattctcc tgcctcagcc tcctgagtag ctgggactat |
| 69121 | aggtgcccgc caccacgccc agctaatttt ttgtattttt agtagagatg gggtttcacc |
| 69181 | ttgttggcca ggatggtctc gatctcctga cctcatgatc cacccacctc ggcctcccaa |
| 69241 | agtgctggga ttacaggcgt gagccaccgc gcccggtcat tttttttttt tttagacaga |
| 69301 | gtctcatttt gttgcccaag ctggagtgca ctggcacaat cacggctcac tgcaccctca |
| 69361 | acctgtcagg ctcaagtgag cctcccacct cagcctccct agtagttggg actacagggg |
| 69421 | cacaccacta tgcctaattt tttttgagac ggagtttcac tcttgttgcc caggctggag |
| 69481 | tgcagtggca caatcttcgc tcactgcaac ctccaccttc caggatcaag cgattctcct |
| 69541 | ggactcagcc tcctgagtag ctgggattac aggtgcacac caccacggcc agctaatttt |
| 69601 | tgtattttta gtaaagacgg ggttttacca tgttggccag gctggtcagg aactcctgac |
| 69661 | ctcaggtgat ctgctcgcct cggcctccta cagtgctggg attacaggcg tgagccacca |
| 69721 | ggccgcctgc atttgcttgt gtccggccag gggatattcc cattctggga ctcattttaa |
| 69781 | aaacattcgc tgtcaggcca ggcgcagtgg ctcatgcctg taatcccagc actgtggggg |
| 69841 | ccgggcgcgg tgactcacgc tgtaatccca gcactgtggg aggccgaggc aggcgcatca |
| 69901 | cgaggtcagg agatcgaaac catcctggct aacatggcaa aaccccgtct ctactaaaaa |
| 69961 | tacaaaaaat tagctgggtg tggtggtggg cgcctgtagt ctcagctact tgggaggctg |
| 70021 | aggcagaaga atggtgtgaa cccgggaggt ggaggttgcc gtgagccaag attgagccgc |
| 70081 | tgcactccag cctgggtgac agagtgagac tctgtctcaa aaaacaaaaa aaaaatcgct |
| 70141 | gtcatcagct catctttctc agcctcaaaa acccaatccc aggctcatcc cacctcccat |
| 70201 | cttgtccatc ttacttctta cagctttgtg actctgcccc ctgaagcttc tggaagatct |
| 70261 | cgggtggcag aaaaggagag agctttcctc cttcatcgga gtatacacgg cggtgtcggc |
| 70321 | cggcaggctg gggaatggga gcagctgcag gcatggttgg cttcaggcgt gttttccctg |
| 70381 | caagccatgg gtaaccaggg gtcaagcggt gggccacatc ccacagagga gatgcgggct |
| 70441 | tgggggatgc cccaggagag ccgcctactg caggagaccc accaagcttg gcggccgtcg |
| 70501 | actgggccct ctccagacac tgctgtgcta gcttcagcat cttggaggtg tcggggggca |
| 70561 | cagtttcccc agcttctggg ggagggacaa gaaggctggt cacagtcggc tctaccactg |
| 70621 | ttacttactg ctgagataaa atgaggcaac ggaaaaggca ggcaagccca ctgcccccct |
| 70681 | cagcaccccc actgaccccg ggcaagccca ctgccccccc atcccctcac tgaccctggg |
| 70741 | caagcccact gcccccccat cccctcaccc cgggtaagcc cactgcccct cccgtccccc |
| 70801 | aactgactga ccctaggcaa gcccactgcc cccccatccc ctcaccccgg gtaagcccac |
| 70861 | tgccccttcc accccccact gaccccgggc aagccgacta ttcctcatgg accccaggca |
| 70921 | aggcccctgc ccccaccatg gaccccaggc aagtcccccc acccacgcat ttctaatcat |
| 70981 | ctgccctggt tttgcctcct gagtctgcta aggctgtggg cccctcatcg aggcccgtca |
| 71041 | cagcctcgac cctaaacagg cctttagtgc tcactcagct tcacttgcaa ctgcaggcct |
| 71101 | ctgggaagtg gccgttttac agagacaagg acgtgctgag gggatcgcct cacccatctc |
| 71161 | ccaccggctg tgtccccaaa gtcctgcctt cccgtagctt ggcatggagc acctctgcgc |
| 71221 | gaccatggac ctgctcacac agcccttctg cgcgccgtgg gagctgcctc tctgtgactc |
| 71281 | gcccctggac ccaccgcccc agcgcctgac ccacctccct gctgcccttc acagaaaact |
| 71341 | tctcatagac tcccattttc tcatcccgcc ccgatttagc ccccgactcc ccccggaaac |
| 71401 | tgcccttacc cgtccccacc ttgccagaca cggtgggcca ctggagttcc tctgtcttct |
| 71461 | gggactttgc tctccccagg ggatcctctg ccactcctcc acccaactcc tcaatcttgg |
| 71521 | agcgccagag ctcagccgag ctcaccggct ccctagggag ctggggcaac tgttagctcc |
| 71581 | ccctgacctg tgtgatccta cgtccagtgg ctgccccgac ctctgtgatc ccacgtccag |
| 71641 | gggctccccc gacctctgtg atcccacgtc cagtggctcc cccgacctct gtgctcccac |
| 71701 | gtccagtggc tcccccgacc tctgtgctcc cacgtccagt ggctcccccg acctctgtgc |
| 71761 | tcccacgtcc agtgggtccc cctgacctct gtgatcccac gtccagtggc tccccctgac |
| 71821 | ctctgtgctc tatgtccagt ggctccccct gacctctgtg atcctttgtg cagcggctgc |
| 71881 | tggcctctca aacttaatgt gttcattgcg gaacttgcta ttcctctcct tgccagtttg |
| 71941 | ctcctgcccc atctgcctcc tgcaagtctg ctctgttcca ccttcaggac gcaccccgag |
| 72001 | gccgtttact gctcccatcc tgggtggtct ggccactgcc atcctcagtc ctcacctgag |
| 72061 | gctccactcc caggctgtcc tctccctcaa agctcaaagt ctcttcaagt ttccctgcac |
| 72121 | accagaacgg ccccagttcc acccaaggca tgcacacagc cccaccccgc ccactcctat |
| 72181 | ttcagggcct ttgcacaccc cacagctgga atgctgcccc acgtctgtgc cgctcactgg |
| 72241 | agccagtgtc ttgccacacc ccagtctcca tcttcttatc cacaacgctg gtcccttcct |
| 72301 | gccactatat cacactgagt gtgggctggt ccacctacta caaatggtca ggctagaagg |
| 72361 | atacaagaga cccccgccga agcctggcac ccatcaaggg ctaggatgcc cacaatttgt |
| 72421 | ccagcatgga ggaagcagca gggctcagcc atgccttttt ctttttcttt tctttttttt |
| 72481 | ttttttgaga cggagtttcg ctcttgttcc aggctggagt acaatggggt gatctcggct |
| 72541 | cactgcaacc tctgctcccg ggttcaagca attctcctgc ctcagcctcc cgagtagctg |
| 72601 | ggactacaga cacccgccac caggcccagc tgatttttta gatttttagt agagacgggg |
| 72661 | tttcaccatg ttagccagga tggtttcgat ctcctgacct caggtgatag cccgcctctg |
| 72721 | cctcccacag tgctgggatt acaggcgtga gccaccgcgc ccggctggct gagccgtacc |
| 72781 | tttagtggtt tccacttctt ctagtaacac ctgggagata tagtggatgc tcctcaggta |
| 72841 | ttccgtgtat gcctcctgtg tccaggaaag agaaagagtg gggtcaggcc agtggaggga |
| 72901 | agtgactcca ttattccggc caggtgccct tgtgggataa gcaaggaaca ccaccacctg |
| 72961 | cttatctctg agatacaccc agagacattg agcctgcggt ggttagctgc agccggaaca |
| 73021 | cggttttctg cgcagggcac aggcagcatc gctgctctcc agacttggaa aagacatcag |
| 73081 | gacctgagag agagagagag ccagggacga gctggacctc attttctcct ctgactgcct |
| 73141 | ttctctccag cacaggaact gatattctca ttcagtgggc ctggggaagt gacctcagca |
| 73201 | gtttgctgtc atttttcatt ctatgcaatg tttccttctt gtgcaactca agggtgattc |
| 73261 | attcagtggc ttaaaaaaat acttttctga gaggataaag ggagtgatac tggaagtttt |
| 73321 | caaatcactt cccggtttaa gttccgagaa atccctttcc cagaacctgc gagttacgta |
| 73381 | tttccagtgc acacaggagc ctagaactgt tacctttcaa ggctgtgtga cccctctgac |
| 73441 | aaggcacagg tcattggaac cacagaatgg tggtttgttt tgttttgttg agacagagtc |
| 73501 | tcactctgtc gcccaggctg gagtgcagtg gcaggatctc gagtcactgc aacctccgcc |
| 73561 | tcctgggttc aagtgattct catccctcag cctcccaagg agctaggatt acaggtgtgc |
| 73621 | accgccacac ccagctaatt tttgtatttt tagtagagac ggggtttcac cacgttggcc |
| 73681 | aggctggtct caaactcctc acctcaggtg atccaccgcc tgggcctccc aaagtgtcag |
| 73741 | gattacaggc gtgaaccaca gcgcccagca atatgctatg tttttttgtt ttatccctag |
| 73801 | ctgttaagtt tcctatcttt aagtctatgc ctcagagaga aagacgaaat ataatccagg |
| 73861 | caggatttct gacttgcggg ggcacctgct gccctaaaaa ccaacttggg gaaagcagca |
| 73921 | gattcaggaa cgaggcttct cctaggcaca ggcaggctcc ttccctcctt gggcagccgg |
| 73981 | gtgtggctgg gccagcttct ctacctggtc gccaacctta gattcggccc catctcagga |
| 74041 | acgggccaca cccctccggc ctgcttttcc ttcctaagac gactgcaaac agcatcacaa |
| 74101 | atgagcaggt atgtggcctt ctctgcatgc cgtcccctgt ggcaaccgga ttaaatctac |
| 74161 | catctctgcc cagaaaaatg cacgaacgca cggacctgcY cgtcactcgg attttggaag |
| 74221 | gtggcctggc ggaccctcct gctacatctc ctctacaggt ggagcccagg ctgtgatgca |
| 74281 | ccggctcagc gagcggagtg gaaagccaag gtccgcagga tctgagctgt ccaaggtcac |
| 74341 | tgcacgcccg gctgcgcccc gcccccgtcc tcgcccggga accgcggccc gggatcccgg |
| 74401 | ctggcgcccg ctcccaagtc tccagcccga gccggccccg tcctcgccct ccccgggcgg |
| 74461 | gggtccctcc aggggccacc ctcagcggcc aagccccgcc cccgccccgc ctcacccggg |
| 74521 | gccggttgcc ggtgtccagc tcgatggccc cgttggcaag cttcatggcg ctctgcagcg |
| 74581 | gcttcaccgt gccgtccccg gccgcagcgg ccatggcgcc gagcggggga ggcggcggcg |
| 74641 | gtagccgagg ggctggacgg gcgaccggag gcccggaacg cagccggcgc tggtgctgga |
| 74701 | gccgcccgag cagcggaccg caggaaggga cgcggccgca cttccggcga cgggccccct |
| 74761 | ccgcgcgtac tgcgggcccc acgggtgtta gtggcggggg cggcagagtc cgggtgggtt |
| 74821 | gtcgcgacgg agccgggcct cttcgccgtc ttgagacggg gctggcgaga agggcccctc |
| 74881 | acggagttgc catgggcgtc taaccgcggc agccaggccc ctctctacgt gagaccccgg |
| 74941 | cccccctccc ctttctgcag cccgcccgcc acctgcgcgc cgcgtggcct ccgccggcgc |
| 75001 | ctgcccgccc cgcgcctccg tctcccacgg taagaggcgg tgagggccgc gtcgcctctc |
| 75061 | cctggaggac tcgtcgtgag gtgtcggcga gcccctccgc ccaccgcccc gccccaggcc |
| 75121 | agcgctccat tggcgacaga ggcgggcacg gccgccctcg ggcccgagag gggagcggta |
| 75181 | cgagcggggg cgctggcacc gcgggtggaa cctcggccgg cggggtcccg cccctggccc |
| 75241 | ctggcccgcc ccgcccgcct cgctttgctt ctcgctccgc ccctcccccg ccccgcctcg |
| 75301 | cttccagcgc gccgagcgga gcctaacgcc gggtcctcta ggaacctcgg gccgggcagc |
| 75361 | acccgcggga ttctgctggc gtcctccgct gccatgaagc gggaccggct gggccgcttc |
| 75421 | ctgtctcctg ggtcgtcccg acagtgcggg gcctcggacg gcggcggcgg cgtcagccgg |
| 75481 | actcggggcc gcccttccct tagcggtggg ccgagggtgg acggggcgac ggcgcggcgc |
| 75541 | gcctggggcc cggtggggtc ctgcggggac gcgggcgagg acggcgcgga cgaggcaggt |
| 75601 | gggtccgcgg cccgggcgtg gcgggttggg gtcgcggcag gggttgctcg tgggaggagc |
| 75661 | cgcactgacg cccggaagcc ggcgcggcct ctcctccacc cggccaaggg cgtagcggac |
| 75721 | tcggggctgc gtcgggatag gcgctgggcg gccggctcgg ttttcctcgc gctccgtgac |
| 75781 | gccggcgtcg ccggcccggc ctccccggcc gcgggctcgg ccagaaggga cccggcgtga |
| 75841 | ggagcgctgt caccgcgggc aggtcgccct ggggtgcccg cgcgtgggaa tcgccctccc |
| 75901 | ctgcttcgtg ccctcgcgga gccggaaagg aggcggcacc tgcaattggc agacaggaaa |
| 75961 | ccgcggcgtt tcctggaggg cgcagcgcct gccccgggtc tcgcggtcgc tgggaggagg |
| 76021 | gtgcagttcc aggtttcact cttgaagttg cttcgctgga gaggccttag aggcctttga |
| 76081 | aatttgaaag atttcttgga ccagcggcca catcccccac agggcggctt gtcccgccga |
| 76141 | gagccgcgcg cccgagccgc ttgctgtgtc cgggagccgc gcgaagggcg ctgcaggcgc |
| 76201 | tgccctcgga cctggagtcc gggacgccct gtgctcagga gcctcctttg ccagctgcta |
| 76261 | acacttcctg ccgctctgtg caggagcagg ccgggctctc gccatgggtc actgtcgcct |
| 76321 | ctgccacggg aagttttcct cgagaagcct gcgcagcatc tccgagaggg cgcctggagc |
| 76381 | gagcatggag aggccatccg cagaggagcg cgtgctcgta cgggacttcc agcgcctgct |
| 76441 | tggtgtggct gtccgccagg accccacctt gtctccgttt gtctgcaaga gctgccacgc |
| 76501 | ccagttctac cagtgccaca gccttctcaa gtccttcctg cagagggtca acgcctcccc |
| 76561 | ggctggtcgc cggaagcctt gtgcaaagta cgccctagtc tgttcagagc acgttcaggc |
| 76621 | tgtcagtact gcagtgtgac gggtgttgag agagggacag ggcgtgcctc cgcgggagcc |
| 76681 | tctgggtggg gggaatgggc catgcccggg ttcagtgcca acagccctgg gactgttgtg |
| 76741 | gagagatgaa ctgggaggag ctggggtgag ggaagagaaa gttgcctatg gggaccgctg |
| 76801 | aggtttgaga tctcgagagg gtcccgtacg acgagcgctg tgaacctccg cctgcttgtc |
| 76861 | ctgctcatgg ccacactgat cctttgcagg gtcggtgccc agcccccaac aggggcagag |
| 76921 | gagggagcgt gtctgggtga gtcctccccc ggtggagggt gggctgggtg ccgaccagcc |
| 76981 | gtggatctga catctctgtt gactctctgc agtggatctg atcacatcca gcccccagtg |
| 77041 | cctgcacggc ttggtggggt gggtgcatgg acatgcggcc agctgcgggg ccctgcccca |
| 77101 | ccttcagagg acactgtcct ccgagtactg cggcgtcatc caggtcgtgt ggggctgcga |
| 77161 | ccagggccac gactacacca tggataccag ctccagctgc aaggccttct tgctggacag |
| 77221 | tgcgctggca gtcaagtggc catgggacaa agagacggcg ccacggctgc cccagcaccg |
| 77281 | agggtggaac cctggggatg cccctcagac ctcccagggt agagggacag ggaccccagt |
| 77341 | tggggctgag accaagaccc tgcccagcac ggatgtggcc cagcctcctt cggacagcga |
| 77401 | cgcggtgggg cccaggtcgg gcttcccacc tcagccaagc ctgccccttt gcagggcccc |
| 77461 | aggtaggagg cacctcttgc tggtgctaga ccaggatgtg tgctcctcag tSgggcaggg |
| 77521 | ttttcagcag aaagtgaatg tctccactgc tctaggtggt ggctggggtg tggtgtgaga |
| 77581 | aggagcagag cttggggctt ctgcctgcgg ctgctcacca catccagaga gcagggaggg |
| 77641 | gcggccagcc tgtctccgcc tcttgctcct ctgtgagcag ggctctgttc cagcagtttc |
| 77701 | tgtggagaca cgctcatcag ccgtcgagtc tttttctaag cctgtgcttg gggctgaggg |
| 77761 | gactgaggaa gagggaagaa tagggccttg tctgtggatg tgggagaaaa gaatggccgt |
| 77821 | tgcctctgcg gctgctgccc tgggccagct gcaggtcatt tcctggcact cagccagccg |
| 77881 | cctggtgaca ccctggtgtg ggccagtgtg gcgcagcttt ctctgctgac aggctgaggg |
| 77941 | gaaacacaag gcccttgttt gggccccaga acccatagca ctgtgggctg gtgcagctgc |
| 78001 | tcctggatgt tgttttagga gggccagggc cagtttccct ccggcctctt gtcctgtgtg |
| 78061 | tgctctgtgc acctgtcccc acctgagcac tcagttccca ctttgagtct tgaggggagc |
| 78121 | ttccgaaaag cgtagctacc tgtaatccca gcactttggg aggctgaggt gggtggatca |
| 78181 | cttgaggtca cgagatcaag accaacctgg ccaacatagc gaaaccccat cgctaataaa |
| 78241 | aatacaaaaa ctagccaggt atggtggtgg gcatctgtaa tcccagctac tctggaggct |
| 78301 | gaggcacgag aatcacttca gcctgggagg cggaagttgc agtgagccga catcgcacaa |
| 78361 | ccgtactcca gcctgggtga cagagcaaga ctctgtccca aacagaaaca cagctaccca |
| 78421 | gatagtttag ctagatacgg gtgcacagat gagacagtgc ggctcggggg cctcaggtag |
| 78481 | gaacgggatt gtgagtgtca catcttagag ctgcttggct ctgtaaactt gtatgcagtg |
| 78541 | tgttgagaac aattctatct atctgtgtat ctatctacct acctacctat ctatcttcct |
| 78601 | acttctttgt ctctctctat ctacctacct atctgtatat ctatgtatct acttatctac |
| 78661 | ctatttatct atttatttac ctacttatct atctatctat gtgtttattt atttttgaga |
| 78721 | cagagtctcg ttttgtagcc caggctggag tgcggtggtg ccatcttggc tcaccgctac |
| 78781 | ctcagcctcc cgagtagctg ggatgacagg cacatgccac cacgcccacc taatttttgt |
| 78841 | atttttagta gagatggggt ttcaccatgt gggccaggct tggtctcgaa ctcctgacct |
| 78901 | cgtgatccac ctgcctcagc ctcccgagta gctgggatga cgggcacgtg ccaccacgcc |
| 78961 | cacctaattt ttgtattttt agtagagatg gggtttcacc atgttggcca ggcttggtct |
| 79021 | cgaactcctg acctcgtgat ccacctgcct cagcctcccg agtagctggg atgatgggca |
| 79081 | cgcgccacta cgcccaccta atttttgtat ttttagtaga gatggggttt caccatgttg |
| 79141 | gccaggcttg gtcttgaact cctgacctcg tgatccacct gcatcagcct cccagaatgc |
| 79201 | tgggattaca ggtgtgaacc accgcgccca gccaagaaca atacttttta aaacttattt |
| 79261 | taggccaagt gcagtggttc acgcctgtaa tcccagcact ctgggatgct gaggtaggcg |
| 79321 | gatcacgggg tcaggagatc gagaccatcc tgactaacac ggtgaaaccc catctctact |
| 79381 | aaaaatacaa aaaattagct ggatgtggct gtggttgcct gtaggcccag ctacttggga |
| 79441 | ggctgaggca ggagaatcgc ttgaccctgg gaggcggaga ttgcagtgag ctgagattct |
| 79501 | accactacgc tctggcctga ccgacagagc aagattctgt ctaaaaaaga aaaaaacaaa |
| 79561 | acaaagaaaa cttattttaa gtagagacag ggtctcactc ttttgcccag gctggtcttg |
| 79621 | aatttctgtc ttcaagtgat ccttctgcct tggcctccca aaaggctgag attataagca |
| 79681 | tgaaccttca ggcctggccg agagaacagt aatatttatt tatttattta tggagtctcg |
| 79741 | ctctgtcgcc accaggcctg gccgagagaa cagtattatt tatttattga tggagtctca |
| 79801 | ctctgtcgcc caggctcgag tgcagtggca caatctcggc tcactgcagc ctctgcctcc |
| 79861 | tgggttcaag tgattctcct gtctcagcct cccgagtagc tgggattaca ggcttgtgcc |
| 79921 | accacaccct gctaattttt ttgtatttgt atttgtattt gttttgagaa ggagtttcac |
| 79981 | tctttgttac ccaggctgga gtgcagtggc acgatatcag ctcaccacaa cctctgcctc |
| 80041 | ccaggttcaa gcgattctcc tgcctcagcc tcccgagtag ctgggattac aggcatgcac |
| 80101 | cactatgcct ggctattttt gtatttttta gtagaaatgg ggtttctcca tgttggtaag |
| 80161 | gctggtctcg acctcccgac ttcaggtgat ccgcccaccc cggcctccaa agtgctggaa |
| 80221 | ttgcaggcat gagttaccac gcccagctca attttttgta ttttttgtag agacggggtt |
| 80281 | taaccatgtt agccaggttg gtcttgatct cctgaccttg tgatccaccc gcgtcagcct |
| 80341 | cccaaagtgc tgggattaca ggcgtgagcc actgcacccg gactgagaat agtaattttt |
| 80401 | aattatttta tttttttgag tcggagtctt gctcttttgc ccaggctgga gtgcagtgcg |
| 80461 | cgatctcggc tcactgcaat gtctgcctcc cgggttcaag cgattcttct gcctcagcct |
| 80521 | cccaggtagc tgggattaca ggtgtgtgcc accatgcccg gctaattttt gtatttttaa |
| 80581 | tagagatggg gtttcaccac gttggccagg ctggtctcga tctcctgacc tcgtgatcca |
| 80641 | cctgcatcgg cctcccaaag tgctgggatt acagacatga gccaccgtgc ctggcctagt |
| 80701 | aattttttaa atgtctcaga acatccacca agtccaggct gtcctagaag gactggatcg |
| 80761 | gagtcagtgg aaggactggg ctttctggtt ctgctgttcc tgagggttct gatgcttgtt |
| 80821 | gctgcctgtg tgcttctgga cgttgggctg gcagggcagg atcagaggct gcatagcacc |
| 80881 | cttggtgggg atggggccaY ggtggggaga acttgagggt cagggacccc agtctccctt |
| 80941 | ctagtcatca atagtagaat catgcctcct tgcaggtgag acctttctgt gctccgtgtt |
| 81001 | ggtaacagga gccttgttct ttgtttctca gggcagttgg gtgagaagca gcttccatct |
| 81061 | tcaacctcgg atgatcgggt aaaagacgag ttcagtgacc tttctgaggg gtgagagaag |
| 81121 | agtgggttta aacagcctaa aatttctcct tcaaaaacca tcagtgacct ttctgagggg |
| 81181 | tgagagaaga gtgggtttaa acagcctaaa atttctcctt caaaaaccaa acagccatcg |
| 81241 | tagggtcggc tgcccatgcg ctggtaccaa actaagccat cacaggggcc ggctgtccgt |
| 81301 | gccctggttg ggacacgcct cctctcgagg tggcgggaga ggaggggctg gggagcttcc |
| 81361 | gaggcttctt gtctctgatt gatggtttcc tgggtgccag agagcactcc cagacctgag |
| 81421 | gcagctccag agggagcctg atagggtttc tgacccagag cagcctagaa atgagtttgt |
| 81481 | tgaaaccact ctggctgggt cctttgcttc tgctactgtt ttagggccat agttttgtgt |
| 81541 | tcgctccaga tgtttctgga gtagagaaac cccgacttta gcccttgact atgacactgt |
| 81601 | tgtggcttga agccgaggct ctgcgaggag gtgttgcagg aggacgggcc tcagaaggga |
| 81661 | cctcatcagt gtctgcaaac tccctgggcc acccctgcct ctgagccaag cgccattcag |
| 81721 | ggcagctgtg ccaggctggg gtttgcaccg tttccaccct ctcctgtaaa ccttggctgc |
| 81781 | aacacagatg cacctcatcc aaatgaggac accccagttt gggggccaat cccagtggga |
| 81841 | tgtgcagtga tgaaatcggg caggtgccag gaggcacctg gggtggctaa gactgtcctc |
| 81901 | aggaccacca ggacacaaaa ccgcagctga ggttatagca taaaggccac aaatcttttt |
| 81961 | tttttttttt ttttcctttt gtgaactttt ttttttagac cgagccttac tctgttgccc |
| 82021 | aggctggagt gcagtggtgt gacctcagct cactgcaaac tccacctccc gcgttcaagt |
| 82081 | gattctcccg cctcagcctc ccgagtagct gggattacag gcgcccgcca ccacacccag |
| 82141 | ctaagttttg tatttattta tttttttttt atttgagacg gagtctcgct gtgttgccca |
| 82201 | ggctggagtg cagtggcaca atctcggctc actgcaagct ctgcctcccg ggttcacgcc |
| 82261 | attctccggc ctcagcctct gagtcgctgg gactacaggc acccgccacc acgcccggct |
| 82321 | aattttttgt atttttagca gagacggggt ttcactttgt tagccgggat ggtcttgatc |
| 82381 | tcctgacctc gtgatccgcc cgcctctgcc tcccaaagtg ctgggattac aggtgttagc |
| 82441 | caccgcaccc ggccaatttt tgtattttta gtagagacgg ggtttcacgg tattggccag |
| 82501 | gctggtcttg aactcctgac ctcatgcgat ccacctacgt caccctccca aagtgctggg |
| 82561 | attacaggag ttagcctctg cacccagcca cttttgtgaa cttagaagtg actcatggca |
| 82621 | aacccatcag ttgcataggg ttcgcagtta tggggtgagg tggtcgattt ctcaaagtct |
| 82681 | gcttagcagg aagctacctt tgagcgcctc caccccttct gggccgctgt ggaaagggct |
| 82741 | gtttccaagt ccagtgtcct ggtgcccaga gcacatggct tgtggggatt gctggcttgt |
| 82801 | gagcctggct ggccctttgt gttcggaaac gggattggaa atttggggat tttagattta |
| 82861 | agacattttc agaagaactc aaatgtggga catgggggtc catttggtat ctttaggcag |
| 82921 | gaggtcgtgt tgggtctgtc actgcccagg cccagggctt tgctgaagat tctctcttaa |
| 82981 | ttcctagaga cgtcttgagt gaagatgaaa atgacaagaa gcaaaatgcc cagtcttcgg |
| 83041 | acgagtcctt tgagccttac ccagaaagga agtaagtggg cagcccgggg tctgctggag |
| 83101 | gcatccgttg ggcgacctag acacccgcct gtgctccagg gcctctcccc ggtgcccact |
| 83161 | cagttcactc tctcgtgtgc ggatctcccg agcccagtga aacgtggctt ccctcagtga |
| 83221 | gcgggaggcg ggggaggggc aggcgggcag gtgagaaggt gggtcctcct ggtgctttgg |
| 83281 | agctgggacc tggtctcggc actgtgaggc tgcctcagag tcccagcagt ggatggcagg |
| 83341 | tgtctctgtc gacagaaccc cgccttgctt gagaaggagg ctcttacgtc ccttctgccg |
| 83401 | gtcctgccct gggttcacac cccagtgtac ccccgaagcc cccaacccgt cgcttctgct |
| 83461 | ttgtgtgcgt ccccagaggc ccgaggaccc agctctgtca tcctcccagt tctgacccca |
| 83521 | tcactgatgt ccatggccag gcagccacgg tccacttccc tgttgaatga cttctgtgag |
| 83581 | cttgttttga ctttgttttt agggattttt attttctctc ttcagttaga acacaggccc |
| 83641 | tggtagtgag ggctggtttt cacttgtgct gttttctttg cagccctata cccagcagta |
| 83701 | cccagaacac tcccaggtag ttggtgactg acccagtccc tgtccttgag catttcacag |
| 83761 | cccagaacaa aggtccagag ggcaggtggc tctgagcacc accgtcagcc actagaaccc |
| 83821 | tgctgtgcag actgagaggt caacggcctt ttttcatgga agttctgatg ctgttaagtt |
| 83881 | ctgaaaagag aaacgggagc agtggaggag gccYggagag agatgcgaag cagagggcag |
| 83941 | gactgagcca gaggggctgc agcagcttca gcatgaacag aaaactggtc aggcccagaa |
| 84001 | aaggtgcccg ggggtcccta cccccaggct tgttcccagg agtcatggct gggagtgccg |
| 84061 | cctgaggctg ctgtacggaa gcccccactc cgtgaccctc Ygggttgagt ctgaatccca |
| 84121 | ggacacctga gaatgtgtct ctggcttgct gcccagaagg tctggggctg ctggtgtgag |
| 84181 | gagggctggg agctgggatg ggaggagcca ggcgagcctt ttccggatcg aggcagattc |
| 84241 | aggaggggag ggtcctctct gctgagcccc tgctgctgtg acagggacag cacactcaga |
| 84301 | ctgtgtcact agtccctctg ggaccccagg agtgcagaca aagacgtgct ttggaacaga |
| 84361 | gacacgggag atacgttttc agaggtctct aataaaaggg gtgacttcag tggaaaccgc |
| 84421 | tgcgcaggag gcaggactgg gtggtagcaa gcatggcctc ctgactgcgg cgagtaccag |
| 84481 | gggcacttgg ttccccagct gccaccgtga gacccccctg ggaacccaga agggcctctg |
| 84541 | cacagccccc ggcccgtgtg ctgcctccca gtcttgtcaa ccctgacctt ggagttctag |
| 84601 | cgggcgtgcc cgcctgtgtc ctcagctggg ggcgcatgag aagagacttc tgtgatgtgt |
| 84661 | taatgagggg tcagtggttc ccgccagagg ggaaggccag ggcctgcacc cccatgggga |
| 84721 | aagaactgac aggcagcagt gaggcctgag cccagcccac ccggtccgca aagattcccg |
| 84781 | tcccaaacac gagggcacag agactgcggg tgctctgaag cctcctgtgc tgaggaggtg |
| 84841 | agtggtcatt gtgggaaYgS catggccagc cactcacaaa caaggggcca cccaatccag |
| 84901 | gcaggcaggg caggtgaggg ggacatgtgg ctaggtcatg aaaaccagtt attagggaca |
| 84961 | gggagagaag gYgcagcctg tgcgaagtca acagttcttc tcagtgcgtt tttgtcttgt |
| 85021 | ttctgagatg gagtcttgct ctgttgccca gactggagtg cagtggcacg atcttggctc |
| 85081 | actgcaacct ccgcctcccg agttcaagtg attctcctgc ctcagcctcc tgagtagctg |
| 85141 | ggattacagg catgtgccac catacctggc taatttttgt atttttagta gagatgggat |
| 85201 | ttctcccacg ttggccacac tggtctcgaa ctcctgacct catgatccgc ccgcctcagc |
| 85261 | ctgccaaagt gctgggatta caggcgtgag ScaccgcgSc ggcctcaatg cactctttaa |
| 85321 | agagaagggc agttagcgac agggtctcat gttgttgcct aggctggggt gccctggcac |
| 85381 | gatcatggct gactgcagcc ctgtgcgctc aagtgagcct ctcacctcag cctcccaggg |
| 85441 | tgStgggact gccaacatga gccactggac tcggcagtgt aaactacttt tttcatgcta |
| 85501 | ttgcatatgt attgttcatt gataatatat agaaacataa ctgggttttg tgtatggatt |
| 85561 | ttgtgccctg caactttgct gaaataatta attagggttt tttgtatgtg gatctttagg |
| 85621 | gttttctacg tagaaggtcg tgtcatttgt gaatagggag aatttgtctt tgttttcttt |
| 85681 | ttcttgccca gttactctgg ctagggcctc cagtgctgcc ctgagagcag tggtgagaga |
| 85741 | atccgtatMt tgttcctgac cttggaggaa gagctttcag tccttcacag ttgagcgtgg |
| 85801 | tgtctgctgt gggtttcatg tagaggtttt atcgtggtga agaagttcct ttctgttctt |
| 85861 | Sagatatttc cttaactatt cttacctgga tccagaaagg aaccacaagt gcgtctctct |
| 85921 | ctttaagtct ccgatagacg cacactggaa gctgctgcat ttgctggtcc ctcagggact |
| 85981 | tgccagacca actaaaatgc acgaccccag acttttggag gacaaggtct cctgcccacc |
| 86041 | atggtcccag ccagcagctc caggaacaca ggctgggctc cctgcagtgg aggcggggtt |
| 86101 | gaggaatgct ggggtggcag ctactcaccg tgtgcttccc agggagcatc acctgttcct |
| 86161 | ctgaagccct gcctgggtgg cttgagcttg gaatcaagct gacccatttt gagagagtgg |
| 86221 | attctgagct ttttcctagc gtaaggtcgt tttgataaag gagtggtcct ttggacatcc |
| 86281 | ctgttctgcc gtcctgatac ctctagttgt gggttttatg tgtagcacag ggtgaagctt |
| 86341 | tagttctcag aacccacccc tgctgtaccc tgtgccctcg ccctccgctg tgtttgccct |
| 86401 | gaccctcctg taccctgcgt cctcgccctc tgctgtgctc acccgaccct gctgtaccct |
| 86461 | gcgccctcgt cctctgccgt ttgcccctga ccctgctgta ccatgccctc gccctctgct |
| 86521 | gtgttcaccc tgaccctgct gtaccctgcg ccctcaccct ctgctgtgtt cacctgaccc |
| 86581 | tgctgtgtcc tgcgcccttg ccctctgctg tgctcgccct gaccctgctg taccccgctg |
| 86641 | taccctgcgc cctcgccctc tgctgtgttc accccgaccc tgctgtaccc tgcgccctca |
| 86701 | tcctctgctg tgccctcccc ctctgctgtg ttcgccctga ccctgctgta ccctgcgccc |
| 86761 | tcgccctctg ctgtgtttgc cctgaccctg ctgtaccctg cgccctcgcc ctctgctgtg |
| 86821 | ttcacccctg accctgctgt gtcctgcgcc cttgccctct gctgtgctcg cccctgaggc |
| 86881 | ctcctgtcca gagttgctct ggctttggtg gcttcaggta gattctgagg gtcagtcagc |
| 86941 | acagaagcaa ctcagggaag cttcagaaaa gaccatttct caggttggag agacaaaaaa |
| 87001 | cattttgcaa atggagatca gaaaccattg aatttgggaa cctctttttt tcagagtctc |
| 87061 | tggtaagaag agtgaaagca aagaagccaa gaagtctgaa gaaccaagaa ttcggaagaa |
| 87121 | gccgggaccc aagcccggat ggaagaagaa gcttcgttgt gagaggtgat gcctgcaacg |
| 87181 | cgaggctcgc cctgcctgtc ggggccgggg ctccatgcat gctcttcatt gcagggagga |
| 87241 | gcttcccacc atctacaagt gtccttacca gggctgcacg gccgtgtacc gaggcgctga |
| 87301 | cggcatgaag gtgagcactg gctgtgcctg acccaggccc ggcagctgtc agtgtgaacc |
| 87361 | tgggggaggt agcaggtgtt tctgcagcct aactcagact tgYgcccaag gacactttga |
| 87421 | cctaggttaa ccgagacctg aagcagtcct agccagtgcc atccttgggg gtagatgtgR |
| 87481 | ggccagactt tcacttgaga caccccccgg ggggtctaga agtttcttgg agtgaagctg |
| 87541 | tgaaggagcc agtggtgtcg ctggaggagg agttggatct gccttccagg ggcctcagct |
| 87601 | gtcacctact gagggctcgt gccatgtggc ccgggtgcgg ctctgagggt ctctcaccga |
| 87661 | gtctctcctt cagaagcaca tcaaggagca ccacgaggag gtccgggagc ggccctgccc |
| 87721 | ccaccctggc tgcaacaagg ttttcatgat cgaccgctac ctgcagcgcc acgtgaagct |
| 87781 | catccacaca ggtacgccta tcgccagtgt cgcccacgcg ggtgacagcc aggggcacgt |
| 87841 | gacctgcttt ccagtcttcc tcatacccat ccccgcccca agagttgggg gtgcgtgaag |
| 87901 | gtggctcatg gggtctttgg tactcagtca cgttggtaga tgaaggctag agtagggcgt |
| 87961 | cagtgtgaat ctgcccttcc ctgtgatgga aggaattcct gacttcccca ggatctaggc |
| 88021 | agtttgtcaa aactaatagc caagcaatct tggtgtcctt ttgcaaattg gtcattttat |
| 88081 | tttttaattt ttttttgaga cggagtcctg ctctgttgcc caggctggag tgcagtggca |
| 88141 | ccatcttggc tcaccacaac ctctgcctcc caggttcaat caattctccc gcctcagcct |
| 88201 | cccaagtagc tgggattaca ggcacccgca accatgcctg gctaattttt gtaatttttg |
| 88261 | gtagagacgg ggtttcacca tgttggccag ggtgatctct aactcctgac ctcaagtgat |
| 88321 | ctcagccttc caaagtgcag agattacagg tgtgagcaSt gcacctggcc caaacaagtg |
| 88381 | tatcttaaaa ggcaaggcag aaggcatgaa ggcacaccag ctctggggag ctgcccacgt |
| 88441 | ggacaggcgg gaggcacgtg tggttctgcc tYttcacRtg tgtgtaggtc gtgtcattaa |
| 88501 | gaccggaggc ggatggggga attatgactg agggtgctgt ctctgtgccc gtcttatttt |
| 88561 | tcacaatttc tataatgcac aagttacttt tataatccaa caaggatatg ttttcaaaat |
| 88621 | gacaaacttg gccgggcgtg gtggctcaca cctgtaatcc cagcactttg ggaggctgag |
| 88681 | gtgggtggat cacctgaggt cagcagttca agaccagcct ggccaacatg gtaaaacctg |
| 88741 | gtctctacta aaaatacaaa aattatctgg gtgtggtggc acacgcctgt aggcacagct |
| 88801 | acttgggagg cggaggtgga ggttgcagtg agccgagatc acaccactgc actccagcct |
| 88861 | gggcaacagt gagacactgt ctcaaaaaaa aaacttgttt gaaccacgtg gacccacata |
| 88921 | tccatggtca gggactcgaa tccctggtat tcagagggct gccttttcct aagtgcaggt |
| 88981 | tctgtagggt ctttgcaggg ctaacttcgg gacttcagta tgagcagatt ttggtaaggg |
| 89041 | gtgggggggg gcctggaacc agctgaggac aactgtatat tgatccttac acacacagtt |
| 89101 | ttccttggtt ttgaacttat tacagaataa gaattattta ttcttacttg aatttactga |
| 89161 | agtatttttt cattaggatt gcttttcact gctgggtcat ctcccgacat gcgggttgtt |
| 89221 | attttttggg ttctccgaca tctctcctga tggcagcact tccttgggtt tggtccttca |
| 89281 | gactctgcat ctgtccggtc actgatggtc acaaccagca gttccttaaa aacctgaaat |
| 89341 | tgggccgggt gcagtggctc atgcctgtaa tcgcagcact ttggaggcta aggcagtagg |
| 89401 | attgcttgag gccaggattt tgacaccagc ctggRcaaca tagtgaggcc gtctttacca |
| 89461 | aaaacttttt ttttaattta ttgatggaaa cagtctcact cttatcacca tggctggagt |
| 89521 | gcagtggcgc gatcttggct cactgtaacc tccacctccc tagttcaagc aattctcctg |
| 89581 | tctcagcctc ctgagtagct gggattacag gcatctgcca ccacgccggg ggtgtttttt |
| 89641 | tttgtttgtt tgtttgtttt tttgactgag tcttgctctt gtcgcccagg ctggggtgca |
| 89701 | gtggcacaat ctcagctcac tgcaagctcc tcttcctggg gtaaagccat tctcctgcct |
| 89761 | cagcctccca aatagctggg actacaggtg ctggccacca cacccaacta atttttcgta |
| 89821 | ttttcagtac agacggggtt tcaccacgtt agtgaggctg gtctcaatct cctgaccttg |
| 89881 | tgatccacct gcctcggcct cccgaagtgc tgggattata agcgtgagcc atgacgcctg |
| 89941 | ggtaattttt ctatttttag tagagaggca ggttttgcta tgttggccag gctggtctcg |
| 90001 | agttcctgac ctcaggtgat ttgcctgcct tggcctccca aagtgctggg attacaggca |
| 90061 | tgagccaccg tgcccagccc ccaatttttt tttttttttc cgggacagtc tgtgtcgccc |
| 90121 | agcctggagt gcagctgtgc aatctcagct cactgcaacc tccacctcct gggttcatgc |
| 90181 | aattctgctg cctcagcctc ccgagtagct gggactacag gtgtgtgcca ccacacctgg |
| 90241 | ctaatttttg tatttttagt agagacaggg ttttaccata ttgattgggc tggggctggt |
| 90301 | cttgaactga ctKcatgatc cgcctgcctt ggccttccaa agtgctggga ttgagcactt |
| 90361 | tgtgtaagcc actgcaccgg ccccccaaat tttttaaatt acctgggtgt ggtggtgcac |
| 90421 | acctgtggtc ccagctactt gagaggttga ggcaggagga tcacttaagc ctgggagttc |
| 90481 | caggctatag tgagccatga ttgtaccatt gcacttcaac ctgggcaaca gagcaagacc |
| 90541 | ctgtctccaa aaaaaaaaaa aaaaaaagaa tcccttgaac ctgggaggtg gagtttccag |
| 90601 | tgagctgaga ttgggctgtt gcactccagc Stgggcaaca gagcaagact caagtctcaa |
| 90661 | aaaaaaagaa cagcgtagag taaaatgcca agatagggtt tcccttcgaa gagaaaagtc |
| 90721 | tgtcttgtga agcgtttcag ggcttgacag atgtggaggc agcacagggt gagtgggtct |
| 90781 | gcatgtccac atgtggcatt tgtggactag caagtgagat ctctttgttc aagggtgtga |
| 90841 | gaaagatttc cagagccagt acagcaatgc atacceccag accctgggta tggacccctc |
| 90901 | tcatatggag atgaattcta gagaacagcc atagctgtga caatcagtat tctatacaac |
| 90961 | cttagtgata cggcttcctt taagaatttg taggccaggc acggtggctc acacatgtaa |
| 91021 | tcccagcact ttgggagacc gaggcaggca gatcacaagg tcaggaattc aagaccagcc |
| 91081 | tggccaatat ggtgtaaacc ctgtctctac taaaaataca aaaatcagct gggcgtggtg |
| 91141 | gggggcgcct gtaatcccag ctacttggca ggctgaggca caagaatcgc ttgagcctgg |
| 91201 | gaggcgcagg ttgcagtgag ctgagatcac gccaccgcac tgcagcctgg gtaacagagc |
| 91261 | gaaactccat ctcaaaaaaa aaaaaagaca agaatctgtg gttaaggaaa tagctttctg |
| 91321 | aggtttcttt aaaaaccatc ctgaaatgca cacagctgat gaagccacgt gacagtgtat |
| 91381 | aaagcagttt aaagatctta ataaacgagg ccctcatagg ccccttgctt gggcccactg |
| 91441 | catggtgaac catgtgcaga aatgtcttcc cagctgtgat ggtttcacat tgtcatcgtc |
| 91501 | gtccccccgg gaggttggag catcaggggc ctggactcac tggactctcc cctctcagag |
| 91561 | gtgcggaact atatctgtga cgaatgtgga caaaccttca agcagcggaa gcaccttctc |
| 91621 | gtccaccaaa tgcgacattc gggagccaag cctttgcagt aagtgtgagt caggaccccc |
| 91681 | tcccagggct gtggccctcg caccttctta tctgcctctg tcccccaggt gtgaggtctg |
| 91741 | tgggttccag tgcaggcagc gggcatccct caagtaccac atgaccaaac acaaggctga |
| 91801 | gactgagctg gactttgcct gtgaccagtg tggccggcgg tttgagaagg cccacaacct |
| 91861 | caatgtacac atgtccatgg tgcacccgct gacacagacc caggacaagg ccctgcccct |
| 91921 | ggaggcggaa ccaccacctg ggccaccgag cccctctgtg accacagagg gccaggcggt |
| 91981 | gaagcccgaa cccacctgag gacggcagtg aggatgagca cctctagcag cctggactcc |
| 92041 | gcagtggctg tgtcagcctc acccttcgtg tgcacccgca tgggagggtc ggagggtgct |
| 92101 | gcccgccctt ggtgctggag gcgggcttgg tgtccggctc aagtagcctt cctctgctct |
| 92161 | gggaccagtg gtttattttc ccgcaaacgc tgagtgactY ggggccggac agttcataaa |
| 92221 | taattgattc ctttccccac taaagcagtc gaggagattt gtaatccact ttttagtgca |
| 92281 | acaagagctc catgttatgc ttgtaataaa ttatttacac gggagctggg ctggtgtgca |
| 92341 | gtggcaggtc ccgtcagaag agatgaggct cctgggacag gtcagcgtca ggggcagcct |
| 92401 | gctgtctgct ctggagggcg gcgctcacct ctgggtcgca gtccccacga tcagccagca |
| 92461 | gctgtgagag aggagcaggt cctcagccca tgccgcccac taggcctcag accacagggg |
| 92521 | aggggctctg gcagaaatag tcgagttgta ttgccagcca ggcaggcaca tggcccaggc |
| 92581 | agctgtcaat tctcatgtcc cccacRtggc ccaaggtggg catcttgacg ttacctctgc |
| 92641 | cacgtgtgag aagctctttt tcgggcaccg aggtattaac tgcagcagaa aaagacgagc |
| 92701 | ttttgttatc agttccacgg ggttgcccta gagagaaaac aggcaaactc acaggttaga |
| 92761 | agacatacag aaacagggct ggtgtgtccc ccatagtctg catgctgtgc cggaacattc |
| 92821 | tttggcagaa ggagcctccg gctgggggga gctcccctgg aggtgggact ggcccttgca |
| 92881 | cctgcctgac ccttgagctc caggctcctg ccagctggag gtgaaactgt gcttgtatcc |
| 92941 | ccagccacga agagctggac cagcttcaag tacatgtcca cagcaacatg caggaaggcc |
| 93001 | tcttccctga tggccgcgtc ttcatggaag taggagagaa gactagaggt aaagacatag |
| 93061 | tgacaaatgg ctacagactg ctggaaaggt agcaggtgat gccaagggat actgctcatc |
| 93121 | tgtggagcag aggcacagac aacccttccc atctggcggg acccagaggt gctgagatgg |
| 93181 | gggtctggga aacactgccc agccctgacc agccctgtgg gtggaggtac ctgtaaaaag |
| 93241 | cgaaaggcag cagcctggtg tgctgatccg gggccacacg gaggaggagc cgccccagcc |
| 93301 | tgaggtctgc aacaccaaga aRtggctcag gcaactctgg acatctctgc ctattatcag |
| 93361 | tgctggggac acccctgggg gtcgggacgt gtaccctggg aggcctggct gtggggatag |
| 93421 | tgtggggcga acagcctgag ctgaggatac ccaggtacct gtcagcagct gggagaggat |
| 93481 | gggggggtcg acctcttgca ggagggtggg tgtggtgcag agagaggcag tccccatgat |
| 93541 | aggcccattg gtcctggggt tgaccagtga gccagtaaat tatcttattg ctttaaacaa |
| 93601 | gtttgtgctt aatctgtccc aactaaaatg gagcttataa acttacttag caaggaacct |
| 93661 | caaggagggc tcgttcttaa ccatttgcaa gatgcctctg aaaagagcgg ccctccgcat |
| 93721 | ttgtgcctca gcagcgtgtt tcttaccact ctctgtcaac tgaaagagtg ccagccagga |
| 93781 | tatcttcctc ttctctaaac actcgaggat tgctgcacaa acgtggaaag cctttggcag |
| 93841 | gtctgtggtg ctctgtaaac cgcaggagac caaccctgag aatggccgac ctggtgctcc |
| 93901 | catgggtagg agggtacagc cctcagcaca gaagagggca tttcctcttt gcttattgta |
| 93961 | agtcttaaaa ctggtgacag ttttacctat agaaggtaat actgggcctc tggacagaag |
| 94021 | aggctcaggt aggaggccag ggacttcgag cacccacacc aaggctgctg caccacgtcc |
| 94081 | tcaaataaga cattaaaaga aaggcccaca ggccggatgc agtggctcat gcctgtaatc |
| 94141 | ccaacacttt gggaggccga ggtcggcgga tcactgaggc cagttcaaga ccagcctggc |
| 94201 | aatatggtga aaccccgtct ctactaaaaa tacaaaaatt agccgggtgt gacagactca |
| 94261 | cgcctgtaat cccagctact cgggaggctg atgcaggaga attgcttgaa cccaggaggc |
| 94321 | tgaggttgca gtgagctgag atcacaccac tgcactccag cctgggtgac agagtgagac |
| 94381 | ccccatctca aaaaaaaaaa aaaaaaaccc acggcctggg agttctcact cacacttccg |
| 94441 | caaacacaag gagctcctga gctagtctgg aaaccctgac ttggaagctg gctgcctggt |
| 94501 | gcccctgcct ggcccacagt gggagaggac accttggctg gtaaggtctg acttacattt |
| 94561 | gaggtcagat gtgacgacag caggcccatc aaggagaaga agaaaaggaa aaccaatagc |
| 94621 | tgtaaataaa aacgtgcact tattattaca ttaaaattac ctgtgctgtc attctaaata |
| 94681 | aggctgacac attcctcttt aattgaaatt ttttacatct aggccataaa tcctttaagt |
| 94741 | ggatcttaga aaactttcca atcacttcta gagagacagc ttaattgaga attaattact |
| 94801 | actggctggg tcatttcaca cttgccttta aacaaagcta gaaacagttt gaaaaagtga |
| 94861 | caagagccag aaagagaaga ctgctgcggt ctccttggct gtgcgcagtc ccaactcagc |
| 94921 | atcatctccc tgcaaggcct cctgtcagaa gtgtttgtaa aatccttgaa gataagccca |
| 94981 | caggctccag cctaaagtag ggcctgactg ggcagggtct ctgtggacac tcctcgctgc |
| 95041 | acactagggt cctaaggccc tggagccact aaaatccctt ctaacaaaac tacacaagaa |
| 95101 | acctgaagac gtactcagaa gaacccctgc agggtggtta taatcaccaa aaccctgggc |
| 95161 | gctgctgtcg atccagcttt gagctggtgt aagttgtgtg ctgacatggc acctatgcgt |
| 95221 | tgcggatctc gggctgggca cagagggctg ctgcctccag ctctcttgtg ggatgcttgt |
| 95281 | ttctgttcct gttctgcaca caccaccctt ggctccctgc acagagagct gtgtgggtgt |
| 95341 | cactggtctt gtgggggcag tcaggctttc cagggtgcag caaccaggtg ggcagtgggt |
| 95401 | ctgagtcgaa catggtccct ggcctggaat tcaagagcag cctctcccac tcctttccct |
| 95461 | atgcctatgg ctgcagacgg tcccatcctt gctgcctctt gtccccagca ccccaggtct |
| 95521 | gagctgtctc actgcaagct tatctggggc tgtccctcag cacatgaatc aaggctactg |
| 95581 | cagcatgaac acagaaggca ccacttgaag gctgacagca tttacgctgg gctgtgatga |
| 95641 | ggataaaaat aagctgaaga aacgcacttc aagcctgggc aacacagtga gacccccatc |
| 95701 | tcttttaatt tttactttaa acaaattttt tttttttgag acaagtctcg ctctgtcacc |
| 95761 | cagactgcag ggcagtggcg tgatcttggc tcactgcagc ctctgccccc tgggttcaag |
| 95821 | tgatcctcct gcctcagcct cctgagtagc tttacaggct atgggattgt aggtgcgcac |
| 95881 | caccacaccc gactaatttt tgtgttttta gtagagacag ggtttcacca tgttgcccaa |
| 95941 | gctggtctca aactcctgac accaagtgat ccgcctgcct cggcctccct aagtgttggg |
| 96001 | attacaggca tgagccacca tgcctggact acagaccccc atctcagaaa aaattacaaa |
| 96061 | attagccggg catggtggca cacacctgta gtctgagcta ctccagaggc tgaggtaaga |
| 96121 | ggatggcttg agcccagaag aggctgcagt gagctgactg caccactgca ctccagcctg |
| 96181 | ggcgacagtg agaccctgtc tcaactacat acaaccattt aaaaaactga aattattatt |
| 96241 | tttactagta aacttgatag tcaaagtctt actccaagcc agctgggcgt ggtggctcac |
| 96301 | gcctgtaatc ccagcacttt gggaggccaa ggcgggagga tcacggggtc aggagatcga |
| 96361 | gaccatcctg gctaacacag tgaaaccccg tctctactaa aaatacaaaa aaaaaaaaaa |
| 96421 | aaaaaaaaag tcttgctcca agccacatat ttgtctttag aaaaacagtt catcagacaa |
| 96481 | gcccagagaa atagcactga ttgaaaccaa gcttgcgaga aaataaatca gtaaaagaat |
| 96541 | ttcctatctt gcctcctctc tctcgcagtc cagcttcttt agctgcttcc tgatgttttc |
| 96601 | ttccctgact tgttgaatcg caaagtgcag tgcagcagct gagagccagt ccgggttggg |
| 96661 | tgcYggggag gcagcctcag gggagaggaa actgggacag agagaacggg gtcattgcag |
| 96721 | ggccttacaa ccatacaacc acgccataga aaccaagtcc ttattcccac ctgtcacctt |
| 96781 | tggggcctgc ctttccatca gaggacagag aagggtttca ggaccatcag aaactaggtc |
| 96841 | tgcatccccc aggtccacgt gagagtgtgg gcaggtgctg gtgctgcccc cacRgcatcc |
| 96901 | tggcgttctg agagctgctg actgagcagg cagctggcct ggtcctgtct accttgccct |
| 96961 | gtgctcttgg gccccagctg cctacatttt ggaagaagag gcctcatttt cctcagttgc |
| 97021 | ccatgcctgg ccctgtggat caggcattcc ctcctacaac agagaacaaa ggactgcctg |
| 97081 | gctctggccc cagtgtggtg tctgagtttt ctggctggtt aaaaaattgg taggaaatta |
| 97141 | atgaaacaat gctgtggttt aagagacttt aaataagctg ggcatgacgg ttcacatctg |
| 97201 | caatcatagc acttcaggag gatcgcttaa gcccagcagt ttgagatctg cctgggcaac |
| 97261 | acagtgagac tcaatctcaa ataaacaaaa acgaggcagg gcgtggtggt tcacgcctgt |
| 97321 | aatcccagca ctttgggagg ctgaggcagg cagatcacct gaggtcggga gttcgagacc |
| 97381 | agcctgacca acgtggagaa accctgtctc tactaaaaat acaaaattag ccaggcatgg |
| 97441 | tggctcacgc ctgtaatccc agctactcag gagactgagg caggaggatc gcttgaacct |
| 97501 | gggaggcgga ggtcatggtg agccgagatc gtgccatctc actctagggt gggtgacaag |
| 97561 | agtgaaactc tgtctcaaac aaaacaaaac aaaaaacaaa acttcagaca atctgatctg |
| 97621 | tgtttgtaga attagaactg gaggtgtgac ctgctttctc cttttttgag atggagtttc |
| 97681 | tctctcattg cccatgctgg actacaatgg catgatctcg gctcactgca acctccacct |
| 97741 | cctgggttca agcaattctc ctgcctcagc ctcctgagta gctgggacta caggcgcaca |
| 97801 | ctatcacacc cagctcattt ctgtattttc agtagagatt gtttcacatg ttggccaggc |
| 97861 | tggtctcaaa ctcctgacct caggtgatcc acctgccttg gcctcccaaa gtgctgggat |
| 97921 | tacaggtgtg cgccaccgcg ccggggccct cttctcttct ttcagcagac ggcgagcagc |
| 97981 | caaaccctga ggggacccgg tgtggcatca gtgattcaaa actagtcaca aagtaaggtg |
| 98041 | ttctccaagg aggcaggatg ggtactagca gtgccctctg acgacccctt aagaacacgg |
| 98101 | cacccacatt cagcaggtgg agtggctgtc agctctgtgg tcctcagaag ggtgtgtgag |
| 98161 | cctggattgc gccaggagct tgatggtttg caaggacccg tgaatatctg actggggtcg |
| 98221 | acaggtcagg gtaggctgag aatctgcatc cctgaggtcc ccatgacact gaccctacca |
| 98281 | gcacgcggtg cactctagga gcccacatta gatttaagag gaaggggagc aggtgcaaag |
| 98341 | gaatcactcg agctcctcac tcagacggga gcctgggaga ggccgttcct cactcagacg |
| 98401 | agacactggc agaggctgtt tttttttttg ttttttttct ggacagagtc ttgctctatt |
| 98461 | ggccgttgga gtgatctcgg ctcactggaa actccaccte ctgggttcaa ctgattctcc |
| 98521 | tgcctcggcc tccccagtag ttgggattac aggcgcccac caccacgaga actcggttct |
| 98581 | tttctccctg ctcacacgag aggctgccca caccgcttct ctcaagcaag ccagggtgtt |
| 98641 | taggagatga ccttgagcag gtcccgaagt gcatctgggc gggcacaccc catctcacca |
| 98701 | cccacacgta ctcgctggca aactgccggc cttcttgtag cttctgcagt tcccggggca |
| 98761 | gcgggctctg gcagtgtctc ctccaccggc agagcagcac aggctccagg ctcggccacc |
| 98821 | acacctatgg agagagcacc agcacacaga tgagggtggc tgagatggac acacctccgc |
| 98881 | tgccccagcc Rtgacaagcc cccagtgctc ctacaggcca ctacaggccc acactcgccc |
| 98941 | tgcgctctgg aactccaaag ccagtatttt ttacgtcaat taaggctcaa agcagttcca |
| 99001 | tgaggcaYtt attacccaca ctcatggaag acgtggaatt tggagaagga agagcctggc |
| 99061 | ctaacgcaac agtgaaggga ccacactccc ctgagctggg aacgaaacag tgaagggacc |
| 99121 | acagtcccct gagctgggaa tgaaacagtg aagggaccac actcctctga gctgggaaca |
| 99181 | aaacagtgaa gggaccacac tcctctgagc tgggaacaaa acagtgaagg gaccacactc |
| 99241 | ctctgagctg |

Following is a FPGT genomic nucleotide sequence (SEQ ID NO: 5).

| >1:74033451-74130850 | |
|---|---|
| 1 | gagatcttct atcaggctat cctggggaac tgctccaggc tgaaacaaac caccagttag |
| 61 | gacgaaccgc ctttgatgtg aaagctggga agctagaatg tagtttcgcc ttatccttag |
| 121 | cgggtcggtt ctaacaattt tgtcacccaa actgaaccac tcaccgggtc aactgggctg |
| 181 | caagccctta tgagttggag acaagKggcc cagccccaac acagtcagac aaatcgctgg |
| 241 | gcggccatct gctcctgaga ccgttgctag agaaacaaga caacatccaa gttctccaSa |
| 301 | tcatggtttt ccgggcgcca gccaaggcgc tccgggggcg tggttacgtg ggcgacgcac |
| 361 | ggagggggcg ggtcagaaac aaccgggcgg aggcgcaccc cagggcgcat gcgtgctgtg |
| 421 | cggcgcggtc tcagggaagg tggggctatg gcagctgcta gggaccctcc ggaagtatcg |
| 481 | ctgcgagaag ccacccagcg aaaattgcgg aggttttccg agctaagagg taccagagaa |
| 541 | ggcaccggag ttctcctggg caatgcagag ggagggtgct tttacgtgcc aggaggtttg |
| 601 | ctggactgtc acttcccgtt tacttctcat ctgYaggcct atgacgctcc ccaggagtag |
| 661 | ctagctaata atccttttga gttttttatt cttttcactg tccctgcttg acatgcttgt |
| 721 | tgcttacttt attcttagta gtatagatat taaaaatgag ggcggaggaa gaagaagaaa |
| 781 | atcttactag tgaagttatg cattgattag ctcattaata aaagttgctt taaataaatc |
| 841 | taaaagatac gaagcacagt attttgaact ttatgagtag atgccagccc cctatctact |
| 901 | tcacgcttta cattacgttc atctacttct gtaatgtaac cgtttgttga gggagtaaca |
| 961 | gaacatggag gaattatgga ttaaaaattN aaaggaagaa atttgttatc aaaaaattgg |
| 1021 | ctatgacact taatccctgt acacctgtct aataaaagtg attatatttg tgttgcagtg |
| 1081 | actgcttaaa aaatactggg actaattctt ggataacagt aaaactttca ggactattag |
| 1141 | attctagctg acaagttctg tttgttaact aattaaaatg ctaaatcatt ttgtttatca |
| 1201 | ataaagagat tccaactgtc tactaaaagt gcacactgtc ttttaaagat gtcatcagag |
| 1261 | agttgggacc ttctgttatc cagaatttta tgttatattc taacagccta tgtaagtttg |
| 1321 | catggttcat cttaaagtaa tagaaaataa agtacccaca tttataaaca gcaacattga |
| 1381 | gggaagggat ctttgggttg tgtctttctt agtcacctaa caccaagtgt ccStgataca |
| 1441 | taagaattga tttgcaaagc tggagatttg tttatgttta tctcctcttc ttattgaaga |
| 1501 | attgaggcaa tagtctttgg caagttaaaa gtcgctctct taatcaagaa acattatgtc |
| 1561 | attcagaatt tataattaaa ttggattgca aaccataaaa ggagggagtg atctaaaaga |
| 1621 | aaaaaaagga acttgtgaac agtcttcatt ataactattt tgaatcgtat taatgcacca |
| 1681 | ctaattattc cctaactttc aaaagccaaa gatacatttt ggaagatgat aatggatatt |
| 1741 | accttcttta tcatctttct caataggcaa acctgtggca actagaaaat tctgataatt |
| 1801 | tttgttttca tttccttgct ttttccaaat aggcaaactt gtagcacgtg gagaattctg |
| 1861 | ggacatagtt gcaataacag cggctgatga aaaacaggaa cttgcttaca accaacagct |
| 1921 | gtcagaaaag ctgaaaagaa aggagttacc ccttggagtt caatatcacg tttttgtRga |
| 1981 | tcctgctgga gccaaaattg gtacgcgctt tatggtcagt tttataatat aggtcctaag |
| 2041 | cagaataatc attgaagaaa aggtttctgt gacttacagt gtataagctg catatctcta |
| 2101 | cccacagctt tacaaacttt aaaatttgaa cataaattga ctttttttag aaagataaaa |
| 2161 | ggaatagcaa ctgttcttac ccattatgac ttaccttgaa aaacttccag aatgttactt |
| 2221 | aaaagaWaat tttattagtt ttaatgaaat tcacactatt cagataggaa tactcctttg |
| 2281 | gaccttatca gtagaaccct tgaggtcctc tccactccag agttcctcca gcaatttaca |
| 2341 | tgaagaaagc aattagtggg aaaaaatact cttgacctct tagggacata atgcagaatg |
| 2401 | caaaagaaca ctgtcacatg gcttaaatat aatacatata tttgaaaacc ctagtttagg |
| 2461 | tcaactttgc cctttgaccg tccattgcaa atagaaataa atgctgtttt tctgaatgct |
| 2521 | cttttctatg tgatacttaa agttgaagcc ccctgatacc agagataaga tatctgtata |
| 2581 | tgacattaat atgacattag cattattcta attagccaac attattttaa tccttacaac |
| 2641 | tatgaggtca gtactgttat tatttctttt tttttttttt tttttttttt tgagaaggag |
| 2701 | tcttgctctg ttgcccaggc tggagtgcag tggtgcgatc ttagctcact ccaagctccg |
| 2761 | cctcccaggt tcaggccatt ctcctgcctc agcctcccga gtagctggga ctacaggtga |
| 2821 | ctgccaccag cctggctaat tttgtttttg tattttttag tagagacggg gtttcaccat |
| 2881 | gttagccagg acggtctgta tctcctgacc tcgtgatcca cctgcctcgg cctcccaaag |
| 2941 | tgctgggatt acaggcgtga tattatttcc atagatgaga atctgaggta cYcaagagtt |
| 3001 | aagtaactgg cccaagataa ctagtgtggt agttatattt aagatttttt tttgggaatc |
| 3061 | caagttctaa aaatcttttt tcgaaaggaa ctctaacttg tgtttttgat tgtgtcattt |
| 3121 | gggagaatta tttaaatggg ctttgtgatt tgtaactctg tatgaatgtt tatatttatc |
| 3181 | agtcttctag tgttacagat tgtgtaattg attcttcttt cttataatta ctattttggg |
| 3241 | gagaatatta cctcatcatt tattactgag ggttgttgtc tgggtttcat tgctaattta |
| 3301 | ttgattggtt gtttgctatg agatgttttt aattttgttc tattgccaat acctttgatg |
| 3361 | tccatctatt tacttattcc tgaaaggcaa atccttaagg ttatataaca cctaaattta |
| 3421 | ctgtcatgac aaagttttag taggtttcta ttaaataaat tgtgcttttt taactttgtt |
| 3481 | tttaaggaaa tggaggatca acactttgtg cccttcaatg tttggaaaag ctatatggag |
| 3541 | ataaatggaa ttcttttacc atcttattaa ttcactctgg taatgttata ctttactaat |
| 3601 | ttacattttc tttttagtct tccaccttta atactttaga gacttttttc tttcttgcaa |
| 3661 | acactttcct tcttttttaa aagaatcttt gaagttattc tgctttgaaa acaaatattt |
| 3721 | tatgattttc atgacttttt ttaggttgta agtagtatag tgattttcac aaattttact |
| 3781 | cgtaaaattg tagatcacgt atatcgtcag tcttttatga atattgtttt gttttgggta |
| 3841 | tgtatataga aaattatttt cctaagacaa ctcattcaaa tatgaatagt tctcgggaat |
| 3901 | actttaatgg ggatttttct tttaatttta atttgtattt taagttccag ggtccatgtg |
| 3961 | caggtttgtt acataggtaa acgtgtgcca tggttggttg ctgcacctgt caacccatca |
| 4021 | cctaggtatt aagccagcat gcattagcta tttttcctaa cactctttct cccttaatgg |
| 4081 | ggattcttgc tttgagggaa gggtggttta acactgtcat tactagactt ttgccagtac |
| 4141 | tttttatctt gcatttttgt gtgattattg Rtagccagag gccacaatgc ttaaaaataa |
| 4201 | aaggaaaatg tgacaagata cagagtggac aagtaaatat ataaaagaga aatcacacac |
| 4261 | acaaaataag acattttaaa acagaaaagt aggaaggaca tggtctcaga atRaagaata |
| 4321 | gctttttaaa ttgaatacat ttagttgtgt gaaaattttg gttacatgaa aacctttgtt |
| 4381 | gtttttctat gtatgttaag gggctaatga aaactgtcat gaactgacac cagtccaggg |
| 4441 | acaggtattt tagaattatt catctacatg gcttccaagg cactttttca ttcttatgat |
| 4501 | ttacatacaa aacaatgatt ctttgtaaag atgatgtcaa agtaaagcat ccaagttctg |
| 4561 | gtttgaagcc taaaaatgtg taaaaMaatc taatttacaa ttttatttaa gaaaataatt |
| 4621 | attatcagtt tagtaaaaat taaattagat accaggaaat gcatagttag ataatttttt |
| 4681 | tatttttatt ttttggaaat agataattct tagtgctaaa cttgactatt ggctgctctt |
| 4741 | tgaaaaaaga aaaaaacaga aacttattag ctctcttttg ttgaacaatg tactgtgtgc |
| 4801 | tgccaagtgc tttacaggta ttattgatct tcagtaataa attaacctta gcttactata |
| 4861 | acatttttac tttataaact tcttaatttt tttgactctc ttgtaataac attcagctta |
| 4921 | aaacacaaac actgtacagc tgtacaaaaa tattttcttt atattgttag ggtttatttt |
| 4981 | gttgttttta tttttacttt ttaaatattt tggttagaaa tgaagacaca aacatattag |
| 5041 | cttaggccta cactgggtca ggatcatcag tatcactacc tctatagtcc tactaaagtt |
| 5101 | caggagctgt catctcctat gataacaatg ctgccttctg aaagacctcc tgaaggacct |
| 5161 | gcctgaggcc attttatgct taactttttt ttgtatgtat catatgaatt tattctaaaa |
| 5221 | taacaatgaa aagtatagta tagtaaatac ataaaccagt aaaatagtca tttattacca |
| 5281 | attattatat actatataca taattgtatg ttctgtagtt tagtaaactg gcagcacagc |
| 5341 | aggtttgttt ataccagcat caccacttga gtggtgcatc Rtgctaggac tttacaatgg |
| 5401 | ctccaacatc actaggtgat aggaattttt cagttccatt atgatcttat gggacaacct |
| 5461 | atgtatatgc tgtccataat tgagYgaaat gttgtaaaga agcaagtatt ttagcattag |
| 5521 | tatattagtt ttctataact tttattttct gtgaagtaga tttttttttt tttttgagat |
| 5581 | ggagtctggc tctgtcgccc aggctggagt gcagtggcac gatctccgct cactgctgca |
| 5641 | agcttcgcct cccgggttca cgccattttc ctgcctcagc ctccagagta gctgggacca |
| 5701 | caggcgcccg ccaccacgcc cagctaattt tttgtatttt tagtagagat ggggtttcac |
| 5761 | cgtgttagcc aggatggtct ggatctcctg aYctcgtgat ctgcctgcct cggcctccca |
| 5821 | aagtgttggt attacaggct tgagccaccg cgcctggcct tctgtgaagt agattcttaa |
| 5881 | ttagaagtct aaacatggtt gaaatttgtt gtttaaaatg tagatacagg ctgggtgtgg |
| 5941 | tggctcacgc ctgtaatccc agcactttgg gaggccgagt tgggtgaatc acctgaggtc |
| 6001 | aggagtttga gaccagcctg gccaacatgg tgaaacttcg tctctactaa aaatacaaaa |
| 6061 | attagccagg cgtggtggca ggtgcctgta atcccagcta ctcgggaggc tgaggcagga |
| 6121 | gaatcgcttg aacctgggac acggaggttg tggtgagcca agatcgcacg tcactgcact |
| 6181 | ccagcctggg tgacagtaag acttcatctc aaaaaaaaaa aaacaaatgt agatgcaaca |
| 6241 | tcatgttttg ggccgtggtc cttcttattt gttatacatg taattcaaaa catttcacag |
| 6301 | attctacagt atatttaaat tttttcctga cctacagttt ttaaaattgt cttatatcaa |
| 6361 | atggcaacaa gaaaatttct cttcgaattt ttaatYtgga tttcattatt ctttcctgat |
| 6421 | ttgcagtaac aagtggcata tattaaatat cgttaaaKat attttgattt gtaacttatg |
| 6481 | ggttataaaa ccctggggtt ttgaaaattt tataaataat actgcatttt taaatggtta |
| 6541 | tttcgtttta attttatagg tggctacagt caacgacttc caaatgcaag tgctctggga |
| 6601 | aaaattttca ctgctttacc tcttggtaac cccatttatc agatgctaga attaaaacta |
| 6661 | gccatgtaca ttgatttccc cttaaatatg aatcctggaa ttctggttac ctgtgcagat |
| 6721 | gatattgaac tttatagtat tggagaattt gagtttatta ggtttgacaa acctggcttt |
| 6781 | actgctttag ctcatccttc tagtttgacg ataggtacca cacatggagt atttgtctta |
| 6841 | gatccttttg atgatttaaa acatagagac cttgaataca ggtcttgcca tcgtttcctt |
| 6901 | cataagccca gcatagaaaa gatgtatcag tttaatgctg tgtgtagacc tggaaatttt |
| 6961 | tgtcaacagg actttgctgg gggtgacatt gccgatctta aattagactc tgactatgtc |
| 7021 | tacacagata gcctatttta tatggatcat aaatcagcaa aaatgttact tgctttttat |
| 7081 | gaaaaaatag gcacactgag ctgtgaaata gatgcctatg gtgactttct gcaggctttg |
| 7141 | ggacctggag caactgtgga gtacaccaga aacacatcaa atgtcattaa agaagagtca |
| 7201 | gagttggtag aaatgaggca gagaatattt catcttctta aaggaacatc actaaatgtt |
| 7261 | gttgttctta ataactccaa attttatcac attggaacaa ccgaagaata tttgttttac |
| 7321 | tttacctcag ataacagttt aaagtcagag ctcggcttac agtccataac ttttagtatc |
| 7381 | tttccagata taccagaatg ctctggcaaa acatcctgta tcattcaaag catactggat |
| 7441 | tcaagatgtt ctgtggcacc tggctcagtt gtggagtatt ccagattggg gcctgatgtt |
| 7501 | tcagttgggg aaaactgcat tattagtggt tcttacatcc taacaaaagc tgccctcccc |
| 7561 | gcacattctt ttgtatgttc cttaagctta aagatgaata gatgcttaaa gtatgcaact |
| 7621 | atggcatttg gagtgcaaga caacttgaaa aagagtgtga aaacattgtc agatataaag |
| 7681 | ttacttcaat tctttggagt ctgtttcctg tcatgcttag atgtttggaa tcttaaagtt |
| 7741 | acagaggaac tgttctctgg taacaagaca tgtctgagtt tgtggactgc acgcattttc |
| 7801 | ccagtttgtt cttctttgag tgactcagtt ataacatccc taaagatgtt aaatgctgtt |
| 7861 | aagaacaagt cagcattcag cctgaatagc tataagttgc tgtccattga agaaatgctt |
| 7921 | atctacaaag atgtagaaga tatgataact tacagggaac aaatttttct agaaatcagt |
| 7981 | ttaaaaagca gtttgatgta gagRtatttt aaatattgta cactttgcct ttttgagtaa |
| 8041 | cattccagag ataggtattt tttgtaggct gtttcactga actcagttaa tgaaaactgt |
| 8101 | attaacataa ttgttgtagc ataatattaa tagtgcaaaa gtacatataa gtcattttga |
| 8161 | tgaaaaatat tccaagacta agttgagaaa agagatacta ttttggatgt gtatcagtat |
| 8221 | ttttgttttt aataatgatt gatttgtgga gcattgtttt ttcacataat tagttttaaa |
| 8281 | ggtaattttc taagcatacc tttggaattt ttccatcttt tttgaggctt ttggtccagt |
| 8341 | gaagttctaa gtattcactg gcacttctct cctcaactgt aattctattt ttaataataa |
| 8401 | aaatggcata ctgtagggtc ttcagagtag tgtaggaata ctgtagaaat actttttcag |
| 8461 | aaacgaatcc atagctgaca aattcactca gtgcccaata tattgtgatt attttcgttg |
| 8521 | ataaagaact agatacaaag acctctgaaa ttgatgataa aatttgtatc tcattaattt |
| 8581 | tatcaaaatg aaactaaaag tacatatgta ttatacactt gaacatgtgt ttgtatatct |
| 8641 | ttaaaatttt cctttatgtt ccattccata ggaaaacaca catatgcaca caaaactcag |
| 8701 | ttatctgtgg ggaaagtggt agtaataatt gagattcatc aataatcata taatttcact |
| 8761 | atagacatta cttgcattat ctcctatcag tccttctaac aaaacttMaa ttaccagatg |
| 8821 | aactgacttt agttttcatc ttattttttg tccacatgct ggtgatgctg agaaacaata |
| 8881 | attggcccaa atgcagacat caagaagcag gcaggaaaat ggaaaaactg agataataca |
| 8941 | caggtgataa acagtttcRa gaaaataaaa attggctaga atctcaagtt atgtgtttct |
| 9001 | atattggtat aagcatataa gtgaaaagtc ttataagtgt aatagagaaa agatttgtca |
| 9061 | gtgtgttttt ttaaatgaaa taactagtct gtgctacttt atgtcaatat aaaaattggt |
| 9121 | aaacWagaag taacttgtcc acaaccctca gttatgatac ttatgtgcgt gttttttttt |
| 9181 | ccaaagtttt tcccaaggag aagatacaaa tatatggtag ctattgttta aaaatgattg |
| 9241 | atttacttgc agatttttca Raggatgtta tgtctttgtc attcattaaa tgttcaaaat |
| 9301 | tgtagtttta ccaaatctga agtgccatca gttataagaa tcaccattat tttatgttcc |
| 9361 | attaagaaaa acacaataaa acatgacact gctttttttg ttacttgaaa ttttcatatt |
| 9421 | tattggagag ctcttttaga tgtttttaaa tacagatttt tatttaaccc tcttgcatat |
| 9481 | acatataaaa taacaaaaaa ataaaatatt gagatatcat caagaaattt ctacagcttc |
| 9541 | acattcaaac tcttcagcac tttcagagct attgatatgc atatgtccac ctagtaccat |
| 9601 | cttctctgct ttcagaatta ttggtaaggc tgcatttcct gagtgctcta ccacacttat |
| 9661 | gtctggattt tttttaccca gctacagacc cacactcctg caagatttga tacccttcta |
| 9721 | tttggcaaaa ctcagtttca cttttctttc agtttaatca caggtcctta gaagatcgga |
| 9781 | cctgtgattc tattcaaagt cagaaagaga tttctcacaa attgaYgtca tattaacatg |
| 9841 | tcaatatgaa aattaattgt ttggtaaacc agaagtgacc tgtctgaagc cctcaattgt |
| 9901 | ggtatttgtg tgtttgtttt ttctttttca agattctttt taaagaaaat atatggtagc |
| 9961 | tgttgtttaa catttatcta cttacagagc gttcagagga tgttatatgt ttattaagtg |
| 10021 | ctcaatactg tattttaacc taatctgaat tttaatcata atcctgYaca atgcatggat |
| 10081 | tctggttgct ttgaaatggt tctgttcaaa ggacttatca atgtctcttg ctttgagatg |
| 10141 | tattgcttgg tgtgtagtag cacaaacgta ccaccaataa tgtacctaat tcaactgaag |
| 10201 | tgacagtcat atacgcagat atgaccaagt tcacatgtga gcaagtaaca actccatgac |
| 10261 | tRttgctgcc tggctaatag ctaYtatact atcatttgta aggcacattc tgttttctgg |
| 10321 | ggtgtcgaaa tgtggataaa aaaagatgaa ttaggaatga ttctaaccta agtatgtgtt |
| 10381 | ttaataaaag gatctaggca aaaaaaaaaa aagtaagtca ctcctaaatt taatccctga |
| 10441 | ataatgcagt gtacatgatg atagaggaga agtgtgtttt tgataagaag tgactaaatt |
| 10501 | ttgtgacaat cttaattttt taggcttttc atttagtctt ccaataaaga taattttttt |
| 10561 | ctgagtttta tcagcagtag aaaaataaga cctacatgga taagtttgaa attaaacatt |
| 10621 | ttgtctcatt ttataatttt tatttttatg aaatcatggc Rcttacatca tatactcagg |
| 10681 | tgtgtaaaag cctgctattt gtaactataa tccataaact tattgatgat ttaataacta |
| 10741 | tcaacaaggg catgggaaaa attcaacatt tgataataat cttttacctt aggcatttat |
| 10801 | tctctaagca aagcagaaat aaaggatatg tatcagtgta tactgattga aataaagctt |
| 10861 | taaaaatcca agtgatactc tgaaattgtc aaaagtttaa tggcatgaat ccaggattaa |
| 10921 | ttcccataac ctgtgatgat ctagtcatct tctgtagcta catagtactc ctaatgttaa |
| 10981 | gattaccaaa ttcagcaata aagccaaaat caaaactgcc ttctgtcaca tatagctgtt |
| 11041 | aagtaaacta cacaaaaKca gagttgctgt ttacatacat catgatgccg tctggacttg |
| 11101 | agcaccttgg cggctcagga ttaaggcacc tcagtctagc ctagcagagt tacaatggag |
| 11161 | agaagtgaaa actcattagt agctcaaaac aagctaaagg atttcagatg ctctttttct |
| 11221 | tgttcatcta aatcaagtct tcatctgtgt caagaactaa attgttaaag acctttttaa |
| 11281 | tttgcttagt gcaagaaaaa taatagcaac catatatcta gttaacttga gtcttctgtg |
| 11341 | cctctaaaat tttgtttaga gaaagcctat ttctattgag gttgaggaac acaattctaa |
| 11401 | tacttagtaa aattattaac attcattgca tttaactttg tcgtattttc cttcctccaa |
| 11461 | tactagtgtg ccaaattctg taacatagta gcatttttca tgctcccatt tatctgtata |
| 11521 | tacagaaact tctgcttagg ttttttttta tatataaaga aagtctagtc ataccagttt |
| 11581 | ggttgaagaa tactaattct gtccctggaa gagctgtcta cgtgagccac aacaaaccaa |
| 11641 | acttgccaca tcattcaaac ttgccaattt ggccgtagtt tagtagatga caactttggg |
| 11701 | tcaggttaat atttggggaa atggaataat aaaagctgct gattttccta ctgtattgct |
| 11761 | acttggagga ttggaggtgt agagggaaga tgcctgtaaa tgagtcactg ttatcaccta |
| 11821 | cctgcacatc agcttgattg ttgacactta tgtctaaaga tgttcatccc Ragagtatta |
| 11881 | ttgtgcatgc aagtaactga ttgatactaa cgagttaaca ttttattttg ggctgcttgg |
| 11941 | acaaaaagtc ttatcttttt gaagcctctg gctgcaggga tctcttagcc gttgcacata |
| 12001 | taaatttcaa ccattcacac agagcaattc atttctgatt gctgtagact agtgcaatcc |
| 12061 | atctgctagt gtcatttcct agctatctgt gccagaatta cactccttca attgtgcttc |
| 12121 | agtgcatttc tgaaacaatc taaactaaaa tgtctgtatt ctttggccac tggcttatga |
| 12181 | tgtgttactg atatgcttgt tgaatctcca gcctctttaa cactttttaa aatctttgtt |
| 12241 | acaaccaaca catcatagct aagtaatctg cacatgtaaa tgattttttt Waatttcttt |
| 12301 | tcttaccagt atccactgtt ttctctctgt agcacattca gatggttcaa gtgagtttta |
| 12361 | atttaccact tccttgtagg agagtgtcac atcgcccaga atagaattaa agcatttaca |
| 12421 | tcttgaggca aaaaaatctt ttaaaataca actatgtaga aatcaagtaa gtgttcattt |
| 12481 | catttgcagg tttagaaatg ttgcactgct taatgaagtt ttacgtgagt ggtttcttac |
| 12541 | ctattatttt tagccttggg gtttgagaaa gtaaattatg tccatcaatt ttctgtactt |
| 12601 | tagagaagtt tcaaatattt ctagaactag aaaacttggg actacacaaa gaaataatta |
| 12661 | tgattatatc aggtggccca agtgcagatt ttaaatgtac aggcacactg acaaagtcat |
| 12721 | aaattaaaaa tgcactaatt atatatcact gggatcaatc tttaatgaat tgaataagaa |
| 12781 | agatcactta tgactggtta aagttgatag agcttctaat taagcattat ccagtcaaat |
| 12841 | aaagagctgt gtatggaagc cagcctatga ggaaggaagg aaaaagtttt agagtaattc |
| 12901 | ttttttactt gttgcaacag ctgattcttc aatacaaaag aaatatttac tttgatttgc |
| 12961 | atttctctga tggccagtga tggtgagcat tttttcatgt ttttttggct gcataaatgt |
| 13021 | cttcttttga gaagtgtctg ttcatgtcct tcgcccactt tttgatgggg ttgtttgttt |
| 13081 | ttttcttgta aatttgtttg agttcattgt agattctgga tattagccct tcgtcagatg |
| 13141 | agtaggttgY gaaaattttc tcccattttg taggttgcct gttcactctg atggtagttt |
| 13201 | cttttgctgt gcagaagctc tttaattaga tcccatttgt caattttggc ttttgttgcc |
| 13261 | attgcttttg gtgttttaga catgaagtcc ttgcccatgc ctatgtcctg aatggtaatg |
| 13321 | cctaggtttt cttctagggt ttttatggtt ttaggtctag cgtttaagtc tttagtccat |
| 13381 | cttgaattaa tttttgtata aggtgtaagg aagggatcca gtttcagctt tctacatgtg |
| 13441 | gctagccagt tagaggatgt ggagaaatag gaacactttt acaccgttgg tgggactgta |
| 13501 | aactaattca accattgtgg aagtcagtgt ggcaattcct cagggatcta gaactagaaa |
| 13561 | taccatttga cccagccatc ccattactgg gtatataccc aaaggactat aaatcatgct |
| 13621 | gctataaaga cacatgcaca cgtacgttta ttggggctct attcacaata gcaaagactt |
| 13681 | ggaaccaacc caaatgtcca acaatgatag gctggattaa gaaaatgtgg cacatataca |
| 13741 | ccatggaata ctatgcagcc ataaaaaatg atgagttcac gtcctttgta gggacatgga |
| 13801 | tgaaattgga aatcatcatt ctcagtaaac tatcacaaga acaaaaaacc aaacactgca |
| 13861 | tattctcact cataggtggg aactgaacaa tgagaacaca tggacacagg aaggggaaca |
| 13921 | tcacactctg gggactgttg tggggtgggg ggagggggga gggttagcat taggagatat |
| 13981 | acctaatgct aaatgacgag ttaatgggta aagcacacca gcatggcaca tgtatacata |
| 14041 | tgtaactaac ctgcacgttg tgcacatgta ccctaaaact taaagtataa taataataaa |
| 14101 | ataaaaaaat taaatattta ccatgagttc agtgagaaga agcaatttac aaaattagcc |
| 14161 | atgtttctag tgtctttgag tatgtatctc taagactaca aacgtgctgg aattttccat |
| 14221 | aacataaaaa taaacaaaaa caaaaaacaa tacttctctt agccatatta tttcatcagg |
| 14281 | aatagtcagc ctagtttcat ctacaatgaa accgaagttt tactagccat catacccaac |
| 14341 | actgaattct agattcagat atgtacctcc ctgcttcgta tccctccctt actggaaaaa |
| 14401 | aatacatagg catctctatt tggagagctc taatcttcta tcaattaagg taatcttttt |
| 14461 | catctagata aatgacacag ccattcacca aattgctcaa ggaacaaacc aggaaatttt |
| 14521 | ccatgactct tcaggtactt cccctcatcc ttttcatcct gttttcaaca agtgaagtct |
| 14581 | atgtatgaaa aacacaacca aaatccatca acctctctca acttgttgag ctaccactgt |
| 14641 | tttctagact aatggaatag tttttttttt ttttttacat cccacaatgc cttattgtgt |
| 14701 | gcagtaataa caatgatMtg acttttataa ttgcacatga acgcaaagtt gcagaaagcc |
| 14761 | tgcaaaggaR agatttagaa gattggcatt ggggtgtatg aggcattgaa agaaaaagga |
| 14821 | atactgtggc catctcaacc ttacttcact aatttggtac tgtttttgta tcccattttg |
| 14881 | ctcccctgaa gactaccttt taggacaaag attcatacta ctttctaaca ttctacactt |
| 14941 | tattttctcc atgaagtttt gcctggtgat agtctgcaat ctctttacca cccacattgt |
| 15001 | tgttttttac ctttttcata tttgctatgt gtaatcagat gtcagctgga aatctgcttt |
| 15061 | ccctttagat gtaggcaaca ttagttgagc ctctattcta aaccccagag atgatgtcaa |
| 15121 | aaacttctct caaatatctg tgaaattcct gaagatcata tatctccata attgctttgg |
| 15181 | actgtaagct tccatttctg gtgagttagt tctgtttggg cttgcatgtg attttctcac |
| 15241 | aattctatga gctgagcgcc tatttttcac tatgtgagtc tcaaactttt cctctttcct |
| 15301 | gcaattaggg catcaccata tcctctccag aaaaaggcct tctcacacaa attttgtgtg |
| 15361 | tagcaaattt ctctgaatca ttggagctag ggaggaagRt gctggaagga aagagaagga |
| 15421 | tacatatcag agcacagcat taagtaataa ttgttcttag aaatcctttg gcatcttcat |
| 15481 | ctccaagact ttcttcacta gctatttttt ttataatcca aaattatgtc taactttttt |
| 15541 | tctcttcctc agaccaactt cttccatccc cttacgagat atgcacacgt tgtttacatt |
| 15601 | gtagacatat agcatttatc tcttctttct cttgtgtcat tagttttctt ccttcaatga |
| 15661 | attattctca tcagtacact aatattctgt gatttttcca gtttaaaaaa aaaaaaccct |
| 15721 | tcactacact tccttcaggt attactttat ttccttgttc cttttacact aaaattttta |
| 15781 | gaatacattg tctgtttttc cattcttcag ttcccgtctt cctatttact cttcaaccca |
| 15841 | attcaggctt ttagcacccc tctccattac cactactcca ttaaacttgt tctgaagatt |
| 15901 | tccagtgctc tcgactttgc taaatccagg ggtcattctt ggtctcatct tcttgccctt |
| 15961 | taaacagcat ttatctcaat tgattacttc tttgacttgc attctttacc tggatgcaag |
| 16021 | gaaccccaca ttactggttc tttttctaac tttctggtca cactttttaa gtctctttaa |
| 16081 | ctgatttctc ctcatcttcc tcacctcata tcactagaag gtcacagata tttgtRtttg |
| 16141 | aatttctcta tccacattca ctcctttgga ataccatcca ttttcatagc atctatgtgt |
| 16201 | tatcaattct caaatctgta tttctcatta tcaaaatgta actctcaaat tgttaaaata |
| 16261 | tgaatccata aattgtgaac atgggtcaga atttaatcag tgaggaagcc agcaggatta |
| 16321 | tgaagttgat ttttatattg ttccRgatgc tacaaggaag aaaatttata tacaacatta |
| 16381 | taaagaattt tttccaataa cagagtacat tttcttatga gataataggc ttaaaagaca |
| 16441 | atttagcaag agttttctca agaagttctt attttagata agaggtagga ttgaggcagg |
| 16501 | aaaatagggc ctggaggcag ggaacgtaag gccaattcac acttaagcta tgacaggaaa |
| 16561 | tatccttttc atagggtgta tgcagagtaa ataactttgt aactttactt cttcctctcc |
| 16621 | atttacatat ggcatacacc aagtaaccag tggaaatctc tagagggtat ttaaaccccc |
| 16681 | acaaattctg taacggtgtc cttgagcccc tatgctcagg cccaYtccca cactgtagag |
| 16741 | tgtactttca ttttgaataa agttcttcat tctttctttg ctttgtgtgt tttgtccagt |
| 16801 | tctttgttca aaatgccaag aacctggtca ctctccacca gtaacaggat tagataatga |
| 16861 | ctgtgggttg gatttcctga aaaccagaat ctgagatgta ctttagaatg caaaatattt |
| 16921 | ttaaggaggg cctgtgggat taatacttgt acaaggaagg ggagagagca aaacagagta |
| 16981 | gaagaaagtg aagctgagat gcaatcccaa agacagcctt aattggctaa tcccaagagg |
| 17041 | agctctggat attagaatag cacttcagtg tttcccaagt tgaacctgat tggccaggat |
| 17101 | ttataccaca catcaattat cttttctcag gtgcccaatt gataaggaag acaatggaca |
| 17161 | agatatctct ttaggaataa gccaatctgt gaaagggggc tgaccaatga aagctgtcta |
| 17221 | ctgacagcac ttgcagtagc tgaggtaaca agcccttcac tgaagaagca atatgggtgc |
| 17281 | catcacagtg tccaccacaa taacattata aagtccactc aattccaaga ttcaatttct |
| 17341 | atatgtaact attttaatga aacattgtta tgttttaatt caagttgaat tatgacttcc |
| 17401 | aaactaatca tgcaatattg tacatatttt aagtgtaccc aatccaagaa gaaactatac |
| 17461 | tttagtagat atgtcaatca ttgatttaca gaagcttttt ttttgtaata ctgtcacaaa |
| 17521 | tctacatgat aatccctagt gccagatttt acttttgact agctgaRcaa gctaaaatat |
| 17581 | tcataaattc acatgaagcc agatggcttc ccactgaata tgcatttgaa tgattcctgt |
| 17641 | acattattta ttccaagaag tgcacaaaat ataccatcgt catagtagag attcYtaata |
| 17701 | agtttgtttc aaaaaggagt caaggaataa caggtgtcca gtgctaacta tattttggac |
| 17761 | tgaaccatga ttctatgtat atttgagaat tactaacagc agattcaata tagttagttt |
| 17821 | ctattagtct ctgaaatatc aggaatctga aataaggaag gttcagcaag aatgactagc |
| 17881 | attttggaaa aaagaggatt taatgtggct agcataattt cttaataatt tgcttagttc |
| 17941 | tctaaaatat tcaaatgtac aacacaaagt attaaccatt tttgtcaagt ctatcagtat |
| 18001 | gtttttaggg gtactttttt caggttaaaa taacagatgt gaatttgaaa tgaaaatata |
| 18061 | acttaatcat taaaagcttt ttctccaaca tatttgggga gagaataaat gaaaatagta |
| 18121 | tttttttctt ttttaattca tagagttttc ctttcattta aatctgctgt gtcccttttt |
| 18181 | aaagtttcct gttctctgga catctgtcat cattgctgtc cccaccttcc atcttctgtt |
| 18241 | tttttccttc aaatgtggta atcaatgttg ttttgtaaaa caaatctaac aattctacta |
| 18301 | tctgaagtca ggatggatat gcttctgatc cttcttcagt gttttgttgc tatgctttca |
| 18361 | tgtgtatctg attgactttg tttgttggtc attgtcctta gcaaagtatt tgcagaggtt |
| 18421 | tcccatggcc taagatgaat ggactctcct gtRgaggctt tgcatttgct tctatcagga |
| 18481 | gaccagagaa gcattgccac ttaagagcaa cctcagatta agttcaagag tgagcttttc |
| 18541 | tgacttagaa ggctctgtat aaatccaaag tgaatatttt tgtagcaata attgctcagg |
| 18601 | aaccttattt tactttcttc tcttttttcc tttcttcttc tttttttaaa tttcttctgc |
| 18661 | tctgcttaac agcaaggcaa gctgcttgac aattctttgg tttgggataa aggaggacat |
| 18721 | atttacttct agctgatWct tatMcaaagg catatttttg ggaggcttta gattattagg |
| 18781 | gtgtcaaaga caccctctaa gaatttccga cctgaggaaa cacttggcta ccctctggtt |
| 18841 | ccttagaacc taagtttaaa tgtgtgcttt tggaaaattc ctcagggcag aaacttactc |
| 18901 | catacgacct acatttctta tacaaagaaa tatccactta tatctctgtg tttaagcatt |
| 18961 | ttcctgaaat ggtttgtgta tacaacttta tggtttttag ttcctcattg ttttttgcga |
| 19021 | ttatgacttt tatacttagc atttaaaatc agtccattat tctcacttat gtgaaacata |
| 19081 | actctagttc tgtattatct tattgactta ttatccaaca ttttcactag tgatatattt |
| 19141 | tagttagtag agggtatggt atgttagtgg gggggatagt actagtagat gatatatttt |
| 19201 | tataatgaat gttacagtca tggaatgtaa tgattatgaa aatgctaaac atatgaagaa |
| 19261 | aaaatgagga ataaagtaaa tatttatacc cttatccctt ttatccccca tcccataagc |
| 19321 | cacatgacgg agtagacagc tcagcaccta gcatgatgct gctatgagat gataaaccat |
| 19381 | gttaaatctt ttacagagca ggtcagaata aacataagca gacaaaagta tgttaaatgt |
| 19441 | atactttttg aaataggaaa taagcctaca aaatttgctt taaaaaagtt accatttata |
| 19501 | gtagatagaa cacatagacc agtgttagac cccctctgac ttcaaattat cacaatttga |
| 19561 | agttaattat gtgccaggat atttgatcag cactaagaat attttatccc ttgctctcag |
| 19621 | ggttcttcca gtttagttga ggataagata ggaaaaaaaa taattgcaat aattcctagg |
| 19681 | tagagctacc ataagtaagg catttgaata gtcagccaaa gagactaaca aaagctacat |
| 19741 | gatgaggaaa gatctcttag tttggtacgc gagagtgctc ggtgcataRc aaagtctgaa |
| 19801 | agaaacccag tcaaagtgta gtgtgtcact tcagaaatca catggaagtg taagagtggc |
| 19861 | ttgggaaaag gtctacactt gattaattta tagtatttta tataaatatg cagataattt |
| 19921 | catatttata taaatacaca ggtaccttag aaattaaacc tctgaccaaa ttatgattaa |
| 19981 | caatttattt caaagaagag gaaagaagag agaaagtcgt atgtgtttat gtgtgtgtat |
| 20041 | gtaatatggg agtgatattc ccagtatttg gctaccaaat gacaaaattg agtttagaca |
| 20101 | ttgatgaata gtaatgaata aggaaacaaa taataataat aattaatgtc ttcccacaga |
| 20161 | tcacttccta ataaaacact gtaaattaaa ggatatggta atttatgtga tagctttgct |
| 20221 | taacgataag cagcacttat tgaactgtgt agaactgaaa caaaagagaa ctggaaaaga |
| 20281 | gaaacttcac aaatgaatta acatagtaca aatattgtac agcagtttat ttactttgaa |
| 20341 | cctgaaaaaa tttaattaga tttcaaaacc ttataaaaac cttattttat Rttctcgtta |
| 20401 | aaacacatat ttcttggaaa tgattttgtt tagacatgag taggtacttt tgtctttaca |
| 20461 | aaagtttatc cttatctgtg ctgagctaaa aattcagtat acagctggtg ttttgattga |
| 20521 | tacagtatag aagtgacacc acatattatt atatcagtct gatttaaaga gctgaaagaa |
| 20581 | ggtgtttatg gagtaaacta cactgcataa actttttttt catggctctt agtagaaaaa |
| 20641 | taacaacagc aacgaccaaa atatgtacac aaatgcacat tttatatttc atagtcagac |
| 20701 | attaactaaa ccaataaaac aatcttcaaa gatttttatt gcataaaaac aaaatataac |
| 20761 | tttctagtta agcattaagc tcaacaaagg aaatgtataa aaatgaaaaa acattttagc |
| 20821 | tttgttccta gcactgctat taactcaccg tgatattcat taaaccaaaa gtcaaatata |
| 20881 | tgaacagcaa gggccactgg gcattgcaag tctgaaatgg tgtgctagaa agtgttttga |
| 20941 | aatttaaagg aaaagaaaaa aaaaaacctt cccgacaggt gaaaataagc ctcctattca |
| 21001 | gttatgatat ctaagaaaat ttaatggaat tattaaagga gcacttacag aatatgtgca |
| 21061 | attactttct ttagatggga caaatatatt tcataactga tttcttcatt tccaagaaca |
| 21121 | gttttatttc tttgatcttt ctttcatgat aatgaggtag atgtgtacag gattgaacat |
| 21181 | tacatttcaa atgtaatccc aacagacttg caacaagcaa attttttatg ttattcttta |
| 21241 | taaaatgata cctctgttta aaccactcac tttcttcaac atactatgaa ttcatatata |
| 21301 | atttgtgtat ttctcccact tacagaacac tggcttatac cactcttcag gtacttctct |
| 21361 | cctccaataa ttatattgtg tttttgtggg gttttttttt tttagctttt ctaagttctt |
| 21421 | ttggtttgat tacatgtttc tttttgtaac tctttttatc ttttctgttc atagtttcta |
| 21481 | ccattgctgc tactttatat aatttctctg tcatttgata tttatgactt tgcctaattt |
| 21541 | agcctcatca atttttagtc cccaaaaaaa atccaagatg aaatttaaat tactttggtc |
| 21601 | ccatcaattt tgtttaattt gtttactgtg taaaacaata atagagattt taatgaacat |
| 21661 | catcttaata agttttctag ttctctgatg tgatataatc taaattgtgt ccagactctt |
| 21721 | gattYatcat attttagata ttaaattgtt atttattttc ccactgtcta agatttttaa |
| 21781 | aaatctacaa gtagcaagac ttattgcaaa tcatgccaga ttcaaaaatc cttctgtact |
| 21841 | actagcccga ttaacccttt cttcctcttt catagcatgt ggaagttttt tttttttttt |
| 21901 | tttgatactc atcaaaagct actttgtaaa ataagttagt tagatttttg aatatcaaat |
| 21961 | taggttgtaa gctctgtaag caagggccct gccttatact tccttataga caagctgtgt |
| 22021 | caaaccactt tttggagttt tattttattt ctaattcaag gacctaacct cagcagattc |
| 22081 | tgatttagtt gatcacgagt aggttcttgt aattgtagtt tgtaaaatct ccctgaaagt |
| 22141 | ctggtttgta agttattgct agaacatcaa atcccttgta taaattacta atgctagcaa |
| 22201 | ctccaactaa tgcaaaaacc tcttaaggat tacatataca ggaagatgag gaaaacaaag |
| 22261 | agactaatat ttgtgtgaat tttccttgca tagaatttca aaagcctgta gtatttaggc |
| 22321 | tgcagaattg caatggccta ctctagtttc actttacctc ttttccgacc ttagcaatat |
| 22381 | tcctcttact gaaggaatgt attctaagca attcaattgg ttcagtccct ttcagttcag |
| 22441 | ttcaatttta ttcagttcaa ttcattccaa aacaattaaa tttatgtcta tacttataag |
| 22501 | gaactgtggt gggctatttc aaatgcaaag atgcctgcat tcaagttact tattgcctgg |
| 22561 | gaggagggat aaaagcatta attctatatt ctataataaa tcccttaata gaagcagatg |
| 22621 | ctgcatggat agcaacttag agtaggattt aaatgtgttt aatttcttgc ctattcttaa |
| 22681 | actacatata tgtattgtat atatgtatat atatttatgt aatgtataca ttgtattata |
| 22741 | tatagtatat atgcacacat ataaaattat agtataaact taccaaatat aaatatgttt |
| 22801 | agtcttcaaa atgaaatagt tactaacagc catcttcagt aacattaaag gatgcagccc |
| 22861 | tgatagatca tctcaaggtg actttgtaaa ttagtacatg tccagactct gtagaagaaa |
| 22921 | tgagttttat ataataagtc aaagagagaa tatatagtct ttaggggacc cgaacataac |
| 22981 | tattgtagtt cactaatagg ttaactgtta tatagctgca ccgagcatca tgggcaatga |
| 23041 | gagtttcaga aagataatga tggagttaat ttgtgaaaac tctcatgtga taatatgcat |
| 23101 | ttctctctcg ttctgatatg cttaaggcct acacattttc caaatgccgt gttacccttt |
| 23161 | accataggcc ctatcattta ttctctggct gtatatatga actagggaaa tatgggtgat |
| 23221 | aattttaggt aactatttgt gtactcagac ctagacctca acctgggaaa ctccggccag |
| 23281 | cagttaggaa atgataacac atggtgagcc aacttaaaca aatagactca agacttgtaa |
| 23341 | tcaaatccag tatattctat tatgatttaa taaaccYagg catcagaaac accacagtta |
| 23401 | gaattgtctt tgcatcattt gctttggacc tcaagagggg gtgaccttaa attataaagt |
| 23461 | tactggtacg ggaagaatga gacccccgga caacacRgag gtgcttctaa cacctttgca |
| 23521 | tgtacaattt ccttatgtaa ttcagaacat gcctcttagt attgatgaca atttcatttt |
| 23581 | taaagtttct agcttgagct taagtccatt aattcacaat ttttcttctc cattagtgtg |
| 23641 | aacttcatgt gaccaaatta tgggtttaag agacaaaaaa gggtatgaga tacaacggga |
| 23701 | gcagcaagga caacatggcc aaaatgttct gtgaagttgg taaagactcc tcagaacagc |
| 23761 | tgacattttg aagcaggaat atgactttcc tggagataaa ggcaagaata ctagagcaat |
| 23821 | cactctccat attaccagac aaggaagtac aatttaggct tactagtagc aagaactcct |
| 23881 | aaaaatggct ttctgtgaaa acaagtggtc ttgaaattca taataattat agtctactct |
| 23941 | tgataatcca tgttgaagaa ggggagtaca gttgtaagga aaacatgaag tttttcacaa |
| 24001 | attgtaatta aaatgtaaat ctttttgtat atacttagat actaaacttg aattctggtt |
| 24061 | gggaagggta tattatttgg atgccaataa aatgagatat aaatctatga ggacatatgt |
| 24121 | tgaattggaa gcactcaact tgggtttatt attaaaagtc tgcctggcat taaggatgca |
| 24181 | ctcaaatatt acttgctata acaagtccct aaataattgg cttcagagac aatagtctct |
| 24241 | gcaaaaagaa atcagaaatc tgtgtgacca tacaaactgc aactttctat taattcacca |
| 24301 | aatgaaatgt gttgatccac taatggggtt atgaatgaaa gctgagtttt tttttttttt |
| 24361 | tttaccccaa cagcattata ctaactccta gcactccttt aaattttatg caagatgtta |
| 24421 | aagattatat atgtggaatt tcaagtatga aaagagccag tctagctcca caactaaata |
| 24481 | tacaatgtat agaaacaaaa tacaattgaa tcagagttag ttcttattgg tggagtgctg |
| 24541 | agcttgccaa tacgatgaag gaagatcatg aacctttgcg tacacacaaa ttgctaatgc |
| 24601 | tatttctgcc actgttatag agaatggtga caagggggtt aaaaaacata gtacatgata |
| 24661 | actataatca ccattctggc attaagaaga tataggagaa gatgtaacaa aaataatggt |
| 24721 | aactacagac agagagctta ctgagacttt ctctgcttgg cgggaataag caaagaactg |
| 24781 | gtcctatgga atcactcatc atgctaactc aaagatctta cataaaccaa gaggcatggt |
| 24841 | gcttagatRa tgctcttctg tctcagggcc ttcaactttt tttttttgag acagggtttc |
| 24901 | gctcttgttg cccaggctgg agtgcaatgg cgcgatctcg gttcaggcat ggcatttccc |
| 24961 | ttaaagttga agaactacca ggaccgattt taattttaag tggccacagt gtagcagaca |
| 25021 | cagcagcttc ctgctttctc tatcactgga gtctgctgca gtaagcaaca gggaattagt |
| 25081 | cactgagtca acaaggggga accacccaaa cttgtttatt taattttgct acataatagc |
| 25141 | tacatatcta tggattctct aacaaccaca ttatatagtc ccacttttca aatctttaaa |
| 25201 | taatttgtcc tgcctatatc aaaatgtatt tcttttcact ttgaggaaca gccacaggta |
| 25261 | gataattgac aagtgaaaag aaaactctca aaaagaagag gaaagagtaa agaggacttg |
| 25321 | gtcatccatc ttcgatacca gctcagtcac ggtagaatat ggcaccagtc agagtcatga |
| 25381 | ggtgcccctt ctaggctcta gcttcaggac aacatttcta aacataccct gggccagaaa |
| 25441 | cgaacctgct gccctgaagg gaaggaccaa gttttggcag gacccatcac ctgctgacta |
| 25501 | aacagccttt gggccctgaa taaccagcag tgatacttag gttgtatgct gtgcactttg |
| 25561 | ggtgagactc tgagacttgc tggattcaga tgagactcag cacttttcca gctgtggtag |
| 25621 | ctacagggag agacaccttc tacttgagaa aagcagagag aaaataaagg ggactttgtc |
| 25681 | ttgcagctta ggtatcagct cagccacagt caggaagagc accaagctct taaggtccct |
| 25741 | aattccaggc cttggctctt ggacagcgtt ttgagacctg ccctgggcta gaggtgagac |
| 25801 | cattgccctg aaagttgagt cccgggccag gtggcattca ccacaagctg acttaacagc |
| 25861 | ccttgggtct taaaggaaca ttggcagtag tctggctgta ctcctcatgg gccagtgatg |
| 25921 | gtggtagtca ctgtgtgacg ttcctctgcc tatggaaagg ggagggaaga gtggaaaaga |
| 25981 | ctgtgtctca ttgtttgggt gccagctcag ccaaagtgca ataaaacacc aggtagactt |
| 26041 | ccaaggtttt gactttagta cctggctcca agatggcacc tctggacctg cacaggacct |
| 26101 | ggagaatctt gccatcctga aaagtaggac acaaacctga ttggctttcc cctcctcctg |
| 26161 | ctgattatag agcccagagc cttgagtgaa cataggcggt agccaggtag aggttatagt |
| 26221 | ggatcttgag tgagacccag tgtgaagccc cagtggtggt ggccacaggg atgcttgtgt |
| 26281 | catcccactt ccagtttcag tcagctcagg acagagagaa agattttgtt agtttgtgag |
| 26341 | aaagtaaggg aagagaacaa gagtctctgc ctaaaaattc agagaattat tccagatctt |
| 26401 | atccaagact atcaaggtaa tacctccaca agtctccaac agttatagca ttaataggct |
| 26461 | tggggtgccc ctgaatacag atgtggctta gacaacaaca cccaagtact ttggaatacc |
| 26521 | tggaaagtct tcccaagaag aacaaatacg aacaagccca gacagcaaag attacaataa |
| 26581 | acaccttact cttcaatgcc caaagatgaa tatccacaaa catcaaggct tttcgggaaa |
| 26641 | acatgatgtt gccaaatgaa ctaaataaga cacgagtgac caatcctgca gagacagaga |
| 26701 | tatttaattt ttcagagaat ttaaagtagc tgttttgagg aaactcaaag aaattcaaga |
| 26761 | taacacagag aactcagaat tcactcagag aaatttaaca aagagattga aataattttt |
| 26821 | taaaaatcaa gcagaaatta tggagtttag aaatgcaatt ggcatatatt gaagaatgca |
| 26881 | tgagagtctt aatagcagaa ctggtcaaac agaaaaaaga actaatgagc ttaaagacag |
| 26941 | gctatttgaa aatacacaga ggagacaaaa gaataaaaaa gaatgaagca tgcctacatg |
| 27001 | atctagaaaa tagcctcaaa aggaaaaatc tcggagttaa tggccttaaa gaggaggtag |
| 27061 | agaaggagct agatagtgat aaaatatcca aagggatcat aacagagaac ttcccaaatc |
| 27121 | tagagaaaca catcaatgtc caaatacaag aaatctatag aacaccaagc agatttaacc |
| 27181 | caaagaagac tacctcaagg catttaacaa tcaaacaccc aaagtcaagg ataaagaaaa |
| 27241 | aaatcctaaa agcagcaaga gaaaggaaac aaataacata taatggagct ccagtacatt |
| 27301 | ggctgaagac ttttcagtgg aaaccttaca ggtcaggaga gagtggcata atatatttaa |
| 27361 | agttctgaag aaaaaaaaac tgttaccgta gcatagtata tctggtgaaa atattcttca |
| 27421 | aacttgaagg agaaatactt tctcagacac actaaggctg aggaatttca tcaacaccag |
| 27481 | acctgtccta taataactgc taaaggaagt acttcaatcc gaaataaaag ggagttaacg |
| 27541 | agcagtaaga aatcatctga aagtacaaaa ctcactggta atagtaagta aaaacacaga |
| 27601 | atattataac actctaactg tggtatgtaa accactcaag tagaaagact aaaagacgag |
| 27661 | ccaatcaaaa ataactacaa caacaaattt ttaagacata gatagtacat taagatataa |
| 27721 | atggaaacaa caaaaaatta aaagtggggg cacaagttaa ggtgtagagt ttttatttgt |
| 27781 | tttctctatg cttatttgtt tatgcaaaga gtgttaagtt gttatcagct taaaataatg |
| 27841 | ggttataaga tagtatttgc aagcctcatg gtaacctcac atcaaaaaac atgtggtaga |
| 27901 | tacacaaaaa atagaaagca agaaaacaaa tcataccacc aaagaaaatc accttcactg |
| 27961 | aaagggaggc aggaaggaag atagggagag aaaatcacag aacaactaga aaataaatta |
| 28021 | aaaaatagca ggaactaagt ccctatttat caataataat attgaatgta aatgggataa |
| 28081 | actttcaaat taaaagacat agactggctg aatggatgga aaaaaagact caataatctg |
| 28141 | ttgcctacaa gaaacatact ttgtctataa agatacatat aggctgaaaa taaagggatg |
| 28201 | aaaaaagata ttctatgcca atggaaacca aaaaagagca tgagtagcta tacttatatc |
| 28261 | agaaaaaaat agatttcaat acaaaagcta aaaaaagagt caaagaagtc attacataat |
| 28321 | ggtatagggg tcaattcagc aagaggatat aacaattcta aatatatatg cacccaacac |
| 28381 | tggagcgctc aggtatataa aacaaacttt gttagaagta aagagagaga taggccctaa |
| 28441 | tacaataata gctggagact tcaacacccg acttttcagc actagataga tagtctagac |
| 28501 | aggaaatcaa caaagaaaca ttggactttg tctgcactat gcaccaaata gacgaaatag |
| 28561 | gtatttacag aacatttcgt caaatggctt tggaatacac attttttccc tcagcacttg |
| 28621 | ggtcattctc aaggacagat catatgttag gttacaaaac atgtcttgaa acattcacaa |
| 28681 | aaattgaaat aatatcaggg atcttctctg gccacaatgg aataaaacta gaaatcaaca |
| 28741 | tgagaaattc tggaaactgt acaaacacaa ggaaattaaa caatatgctc ctgaatattc |
| 28801 | agtgggtcaa tgtagaaatt aagaaggaaa ttgaaatatt tcttgaaaca aatgttagtg |
| 28861 | gaaacaaaat ataccaaaat ctttgggata cagcgaaagc agaacaagaa ggaagtttat |
| 28921 | agcgataagt gcctacatca gaaaagtaga aaaacttcaa atacacagcc taatgataca |
| 28981 | ccttaaagaa ctagagaagc aaaaccaagc caaacccaaa attaatagaa aagaaataat |
| 29041 | aaagactaga gcaagaaaaa tgaatttgaa ataaaatgtc aatgaaacaa aaagggtttt |
| 29101 | tcttgaaaag ataaacaaaa tgtacaaacc tttagccagg ctaagaaaaa aagagaaatg |
| 29161 | atctaaataa aaaatgaaaa aggaaacaat acaactgttg tcacagaaat tcagaggatc |
| 29221 | attattggct actatgagca actatatgcc aataaattgg aaaatataga agaagtgaat |
| 29281 | caatttcaag acacatacaa cctaccaaaa tttaaccatg aggaaatcca aaacctaaac |
| 29341 | agacgaataa caagtaaaga gaccaaagca ataataaaaa gcacctccca gcaaagaaaa |
| 29401 | gcctggactc aatggtttca ttgcttaatt ctaccaaaca ttttaaaaaa gaaataatac |
| 29461 | caatcctact ctaactgttt ggaataatgg aaaaggaagg aatacttcca aacttattcc |
| 29521 | atgaggctag tagtaccctg atactaaaac caaagacctg tcaaaaaatg aaaactatat |
| 29581 | gccagtatca ctgatgaata ttgatgcaaa aaccttcaat aaaatacgag caatccaaat |
| 29641 | tcaacaacac attgaaaagg tcaccatcat gaccaagtgg gatttatctt agggatgcaa |
| 29701 | gaatggttca acatatgcaa atcaattaat gtactacatt gtatcaacca aattaagtac |
| 29761 | gaaaatcaca tggacatttc aattggtgct gaaaaaccat ttgctaaaat tcaacatctt |
| 29821 | ttcatgataa aaatcataaa actggatatg gaagaaacat acttaaacac aataaaatcc |
| 29881 | atctatgaca aatccatagc tagtatcatt gtaaatgata ggctttcctc taagatgtgg |
| 29941 | aacacaagga tgcccatttt tcaccactgt tattcaacat agcactggaa ctcctagcta |
| 30001 | gagcaatcag agaagaggga aaaaaggact tcRaaattga aaagaagcaa gtcaaattat |
| 30061 | ccttatttac acatgatatg atcttatatt tggaaaaacc taaagactcc acaagaaaac |
| 30121 | gacttgaatt gataaattta gtaaagttgc aggatacaaa atgaacatac acaaaaatca |
| 30181 | gtagcatttc tctatgccaa cagcaaacaa tctgaaaaag aaatcaagaa attaatccct |
| 30241 | cttatagtag ctacaaataa aattaaatat ctaggaatta atgaaaaaat tgaaagacct |
| 30301 | ctacaataaa aactaaaaaa cattgatgaa ggaaattgaa gacacacaaa aatgaaaaaa |
| 30361 | ttgtccatgt ttgtgaattg aaaaaattaa tattgttaaa atgttcatac tacccaaagc |
| 30421 | aatctacaga ctcaatgcaa tccctatcaa aataccaatg acattattca gtgaaataga |
| 30481 | aaaaaaaatt taaaatttat atggaaccac aaaagaccca gagtagccaa agctatccta |
| 30541 | cgcaaaaaga acaaaactgg aggaatcaca ctacttgact ttaaattatt ctacagagct |
| 30601 | acagcatcca aaatggcatg atactggcat aaaaacagac acatagatga acggaacaga |
| 30661 | atagacaacc cagaggtaaa ttcatacatc tacaatgaac tcatttttga caaggatgcc |
| 30721 | aagcacatac attgggaaaa ggacagtgtc ttcaataaat ggtgctggga aaactcggta |
| 30781 | tctatatgca gaagaaagaa actagacctc tatctcttgc catatagaaa aattacatcc |
| 30841 | aaatggatta aagacttaac tacaagacct caaagtatga cactactaaa ataaaacatt |
| 30901 | ggggaaactc tccaggacat tgaactgggc aaagatttct caagtagtat cccatgagca |
| 30961 | caggcaacca aagcagaaat ggacagatgg gatcacatca agttaaagag ttgctgcata |
| 31021 | gaaaagaaac aatcaacaat gtgaagacaa cacacgaaat gggtgaaaat atttgcaaac |
| 31081 | tacccgtctg atagggattc attaatatcc agaattttat aaagagctca gacaagttta |
| 31141 | caggaaaaac atcaataatc ttatttaaaa atgtgcaaaa tatttgaaaa gatatttctc |
| 31201 | agaagaagac atacaaatga caaacaggca tatgaaaagg tgctcaacat catttataat |
| 31261 | caggaaaatg caaatcaata ctacaatgag atatcttctc acctcagtta aaatggctta |
| 31321 | tatcaaaaag gcaataacaa atgctggcaa gaatgtgcag aaaagggaat tcttgtacac |
| 31381 | tgttagtatg aatgaaaatg aatacaacca ctatggagaa cagcttgtag tttcctcaaa |
| 31441 | aaaataaaga ttgagctatc atataatcca gcaatcccac tgctgaatta catatacaaa |
| 31501 | agaaaagaaa taagtgtatc aaagagataa cttcactcct atgtttgtgg catcattgtt |
| 31561 | cacgatagct aagatttgaa agcaacctaa gtgtccatca atagatgaat ggataaagaa |
| 31621 | aatgtggtac ataaatgcaa tggagtacta ctcagccata aaaaagaatg agatactgta |
| 31681 | atttgcaaca acatagatgg tattggaaat cattatgtga agtgaaataa gccacacaca |
| 31741 | gaaagacaaa ttttgcatgt tctcatttat ttatgggagc taaaaataaa agcagttgaa |
| 31801 | ctcaaggaga tagagaatac aagagtggct accagaagct gggaagggta gcaggtggtg |
| 31861 | gggttgggga tggttaaggg gtacaaaaaa tagttcaaaa gaacgaataa gacctagtgt |
| 31921 | ttgttagcac aacagggtga ctacagtcaa aaataattta attatacatt tgaaaataac |
| 31981 | taaaagtttc taatttgatt gtttgcaaca aaacagataa atgcttgatg ggatggatac |
| 32041 | cctatttgcc ctgatgtgct tattacgcac tgtatgtctc tatcaatata tctcatgtgg |
| 32101 | cccttaaata tatatatatc tgctatatac ccacacaaat taaaattgaa aaattaaaaa |
| 32161 | aaagaaaata aaaattgaga aaagaaaaaa aaactctcaa ataagtagaa ctattatttt |
| 32221 | gttttccctc cccccaattt tttatgtttg tttattatac gatttagaaa cctctaatta |
| 32281 | gagaaacctc taatttttaa aacaaacaaa cataaagttg attacattag attctaaata |
| 32341 | acatttttaa aaatgttatt tgtccaaaac aataattagt tattaattga actataatta |
| 32401 | tcacacaata tcatttttaa gctggttaat taactcttag gatgggtgaa gaaagaatta |
| 32461 | agaaggaacg gaagtggcct gagtcatgaa gtttagatta ttctgattat tttaattcct |
| 32521 | caaagaaact ttttctgatt gaaatttagc ctttaataga tggactttct catgccaatc |
| 32581 | atgtatatct catcatagga caaatactgc attgcggtta cctgtttact catgcatctc |
| 32641 | tggccccatg agctctgtgg atttcttggt acaatacgta tttgttgaat ggataaatgg |
| 32701 | actgatcagt tcgtaaattg agctagactg acaattcaaa gaagtcatat aaatttggaa |
| 32761 | aagaaatttt agccagttac agaaatgtga ctcttacctc tatttaatct agattattta |
| 32821 | atcaacttta tttcatgttt acccaccaac aaaggaaatc acaggaaaaa ctatgatctt |
| 32881 | tgcatttgct gttatacttt attggacaca gaaatagcaa tattgtttat tcctttttaa |
| 32941 | aatatactct gacagttaaa gatactgaga acacatactt aaattactgt gacattatat |
| 33001 | tatcatatta taaatatata ttcattataa tgtattctca agtcttactc atttttgagg |
| 33061 | attaatctga atattacctc aacatgttca gactgatgat ggtgatttat acatcatcca |
| 33121 | acatactaaa aacttagata ttctagtgaa aaagaaagaa aattgtttga taaataagac |
| 33181 | tcttacaaat tatgcttgtt taaatgcaga aacacattat ttaaaagaaa attaatcaaa |
| 33241 | taattgtgtg cacttgaata gttgtatgca tcttaggcaa tggtcaattt atttttaaaa |
| 33301 | taattttatt tcaacacaaa ttatttaatc tttattaaaa ataaaggaaa aaaacctaaa |
| 33361 | tataaactaa ctagtaattc caccttctta tgtttaaagc aatgattaat ttgtcaacca |
| 33421 | gatgcaacgc attgaaactc tatgcttccc caaaacatgg aaatatatta ataaggacta |
| 33481 | atatacaatg aagaaattat caaagtataa attaattcaa cttctaaatg tgataataat |
| 33541 | tgttccaagt gcattagttt gatttcatgg gatacatttt cttttatcat ttctctgttg |
| 33601 | gaaccataaa ggtagaagtg tcaaggtcaa tttttacgtt tttcattttg aaagcatcct |
| 33661 | tttaaatctt tataaagaaa tcagtataaa ataaatataa atagcagatg cgtaagtgta |
| 33721 | gattgggata aataaatcgt ataaacccca aataaaataa tgcagaaaga acataagtgg |
| 33781 | gtttattgaa ggagtattag aatataacta tttgtatgca gaagtggtct gagaaatcag |
| 33841 | ttgaaaagac agaggcatta aaactagatt taggcaattc agtagaatat caaacttagt |
| 33901 | tgaaacaaag tagttgaaga aataatattt cataaatgtg ttctgacatt ttctaatgaa |
| 33961 | gaaacgagca agcataaaag aaagaaacct tactattaat catgtatttg agttggcttt |
| 34021 | tctttataaa ttattaaatt tccttgagta cttcaaatgc tctttttaag gaattattat |
| 34081 | tttttgcatc atttgtttta tatttttcca taatttttta aattaatatt ttgttagaat |
| 34141 | tgttttcagt ttacaaaaaa catggatcag aaagtacaga gttactatat actcccccta |
| 34201 | cacacacaca atttttctta ttaacatctt atatagtata tttgttaaca tgaatgaacc |
| 34261 | aatattgata aatgattttt aactaaagtc catagtttac gttagtgttg tgtagttcta |
| 34321 | tgatttttgc caaatggata atgtcatgta tccaccatta gagtatcata cagaatattt |
| 34381 | ctctactcta aagatttcct gtgcttcacc tactcatctc ttcctctcct ctacccctgt |
| 34441 | aaccactgat ctttttatgg tttgtatagt tttgcctttt ccaaaatgtc atatagttga |
| 34501 | aatcacacag gatgtagcct tatcagactg gcttatttca cttagtgata tacatttaaa |
| 34561 | gttcttccat gaattttcat ggctcctttc ttttcattgc tgaataatct tccatattct |
| 34621 | ggatatacca cagtttgtct ctccattcac ttatgaagga atatcttggt tggttcaatt |
| 34681 | tttgtgaatc acttttaaaa tacgagacta tacttgcata gccaaatcat gtattgcccc |
| 34741 | aggaattacc acaggataga actctttgat aatctgaagt aatctttctt ttgtttttat |
| 34801 | tctacttatc tcttttctct tgcatgaaat tcaaaggtaa aaaaatttct gttaagtagc |
| 34861 | atctcacttt aagcaacctt aaaatagtgc ttcttaaatg atgctccaag tagcatctac |
| 34921 | attagaatca tcagagttgc ttgttaaagt tcagattcct gggctctact cttgatgtac |
| 34981 | tgaatcagaa tttctgggga tggagcccag gtttctattt ttttaaacaa agctcctcat |
| 35041 | gttgcctacc ctcactaaac tttgaaagca gactactttt gtaatcaagt tgtgtttttg |
| 35101 | ataatgtatg atataacatg ctttctccac ttttggacgt ctcaggactg gggaaagtag |
| 35161 | gattcaactt agagtatgtg gccaactctt tattagaaag aagtactttt tacagaaatg |
| 35221 | tactgagtat tagggaaaat attttttgat tctatgttgt ttctttccaa cattctttgg |
| 35281 | aaaaggaatg caaggatatt atcctggacc atttacattt ccagcatttc ttcatcaggc |
| 35341 | tgaaggtacc cagaaaaaat tactctagta cctcaaacca ttacctagat ccacattctt |
| 35401 | aagaagggct tagagataaa tattgtttct ttcaaaccag gaaactgtta tagttcaata |
| 35461 | gtggttaata ttttgggtaa tattttgagt aattttctgc atttcttata ttgctatcgt |
| 35521 | ttcattttat ttccagcaaa atgtattgca ttagaatgtg ggagaattca actgagttgc |
| 35581 | tgaKtggatt ttcaaactca tcgcagaaat cacacctttg aaagccaaag ctttcatttg |
| 35641 | taaaacctag gctttttaag agaataaaag tattcttgaa aatcaaaagg aaaaattcta |
| 35701 | aaaagcagta catcagactt gtgtcccaag caggaaatta gaaacacagt ggaagatgat |
| 35761 | gtaagagcta atttttcctc catatgtgga aaacatacat gtacacacac acacacggat |
| 35821 | ggaagatccg agatattaga aataagaaaa gagatcaaga aaaatatatt tagttgtcct |
| 35881 | ctagaaggct cccatctttg tccttctgga ttcagagtat agaaaccatg attattttaa |
| 35941 | gaaactctga gaggagaaag agctggtgca ttatttccta aagtgtagcc ctgggagatt |
| 36001 | gcaagtcttt taaagaaagc tatccagtat ggtcttcaag ataaggaaga agagattaag |
| 36061 | caggaaggtc cctggacaaa acaaagactt tccaaactga gaaaatgggg gtcacagaag |
| 36121 | tgcttagaga aagctggtgg aactgaagat gtctggtttt caaacaagga gaatgtagag |
| 36181 | tgtatggacc aaggtaacca gatgccactg cagtatagca agtatcaaca gctgtggtag |
| 36241 | gcatgtactg ataagaaggg aaaaggagaa tatgtccaga tgaggacaaa aagtgtctat |
| 36301 | gaagccagac acctgatgcg ccaactatat ctcatcagtc tggaagcctt acctctgaaa |
| 36361 | cttctattac ttttgctgag caaaaatata tggtgaRtcc taactttgtc taatcattta |
| 36421 | ttcaaaaaga gagttgtttg aacatttaac tatagaaaat atgaaatctg tatctgttaa |
| 36481 | gttaaagcta tatcctctgg atgtgaatct gaagattaaa atttatactt tctaggtata |
| 36541 | agaagattat ctgaaataaa tatatagtga aaaatttaat gttttctttc tgggtttccc |
| 36601 | atttattctc catcatagac tgggttcttt atatcagtag aaataggcat acaatatgca |
| 36661 | tacaaagaga aatgtcctaa taaagtaaat ggaaggtgca tgaaagagaa attctggaag |
| 36721 | acaaatcaat aatatYtagc ttggatatta gagtcacatg gtcagaactg attccagact |
| 36781 | tccaacttac tagtaacttt gtgttttctc atccataatt tgtgaataat atctacctag |
| 36841 | cagcactgta tgtatgagat taaataatga gatatatgta aagcatgtaa actagggtct |
| 36901 | gacacatagc aaatgctcaa ttttcttatt cttaatctta ctgttcttgt catttattat |
| 36961 | tttacgaatt gctatttgta gaacttaatg ttttacattc tgatctatga ttcttatggt |
| 37021 | cggaataatc cacaatagta taaatcttgg tctatgttga gaatttcttc atacaaacat |
| 37081 | aattatgtag ctcttataca ggccaaacct gcattcattt ctattagtta tatgctagag |
| 37141 | gtaatgaatt tttattattt atttatgtat gtagacacat tctattacca ctctttacca |
| 37201 | actgccatta ttttggctta ggagaaaatg atcttgaaaa caggtctatg tagttttaac |
| 37261 | agctcttttg tgaacttcgg atgtaactga atcattttct agatttatct gttaaataaa |
| 37321 | tggtccatga aagtatgtat gggtagataa ttgatggaag caaataatcc tattgaaaca |
| 37381 | tttgctattt atttttgata cagttctttt aaacagactg attagttccc tttatgattt |
| 37441 | caccctcgaa agcacacacg attttgttct ttatcattgt tgcatgacat tccatttggc |
| 37501 | agttgggctt atttttagcc agactatcaa agtatttttc aactggactg tcactgcact |
| 37561 | tgaacttgga atcttataac ttgaagaact gYcctggaga aaggaagaaa cttataataa |
| 37621 | atgggaaatt ataaatctag accaacccaa acttgtactg gtaattattc taattattct |
| 37681 | tatttcctta agtgtaatat ggttcaatta tagttactga taactaacaa ctcattggaa |
| 37741 | tatgtgttaa tttgtatacg gaagataagg cagcatgttt tgtgaaagtc tttgaaaaac |
| 37801 | tatctgaact caaattattt ttttctgaag atctttatag gaaacaagaa atttcttaca |
| 37861 | aaaatattat tttagttcct tggtataatg ttacagaaaa aaatttggaa tattgctgaa |
| 37921 | tgtagggatt ttagaattat atcttaagta tctagtattt aaaaactaaa aaccgtcttg |
| 37981 | caactagaga ttgctataat taaaatgcaa taaaatcaaa tgaaataagt caaatatatt |
| 38041 | tttctatgta aatatattat ccaatatcga atgggttctt cattactaca attcaatatg |
| 38101 | attgcatttt aaatataaac atctgcattt taaacWcaca atcagtaatt taaattaagt |
| 38161 | attgtagaat ctagaataaa aaacagtttt gattacttgt atgttatgat ctgtgtctac |
| 38221 | attttgcaaa acacaactat gaatcaatct catttgttct tctttactag atgaatggaa |
| 38281 | gaaaaaagtc agtgaatcat atgttatcac aatagaaaga ttagaagatg acctgcagat |
| 38341 | caaggaaaaa gaactgacag aactaaggaa tatatttggg taaagttgta agagtcatta |
| 38401 | tttctttgta taagtgtctt cagtattcac cttaWttttt aaagtatctg tatatttttt |
| 38461 | aaacccgtag aacctcagac ataagcagtc atgttctaag ccttaggatg tcagattatc |
| 38521 | tctctttgac cactcatata gctctcaaaa tgcaaacata acatctcacc tccttaaaat |
| 38581 | ataaattgta atctgccatc attaaagttt aaaaataaaa acctaaaggc tagcctttga |
| 38641 | atgcaagcta ttgttgatct cagtaatctt ctcaggaatt agatcattct tgtaaaccat |
| 38701 | gccggcaatg aaaagcaaga tttttcttcc cacatagtgg tcgcatattg gccacattgt |
| 38761 | tctctttgaa ctatgacggt aagtccaatt ttaacaaacc ctctggtctc ttgacattga |
| 38821 | aaggctgtcc cttaatggaa ctgagtatca ttctgcccct gaacaatcat ataacaacat |
| 38881 | ataatattgc cttcaggatt tcagtagatt ttgttccatg ataacaagag aaaaataatc |
| 38941 | taagtgtcac aaccttggaa gacacaaaat gttcagaaaa gaggagcaag acccttatat |
| 39001 | acacaaaaag ccttctggtt aaattgaatc tgacattgag aaactagatg cccattagag |
| 39061 | ggtcaagaaa gttacctgcc tgactaagga aaagttccat ataggccaat tagccatgtt |
| 39121 | catttatagc tgtgttatgt aatgcactca tggcaaagaa ctccctctat tttacttaga |
| 39181 | aaacaagtgt aaagcaatga ccaaatgaca catctttaaa aYaaaatttt agatatagta |
| 39241 | aacatataaa tatcacataa ttggcaaatR aaatcaccca tgtataatat agtttatatg |
| 39301 | aaattcgtca atggatgtaa caaggctttc acaagtgttc aacttatggt tcaaaaagta |
| 39361 | gttggttctc tttatagttt ggcccaggat gatgaactca acactgacac ttgtgatgag |
| 39421 | ataccactga tctgactttt gcaagtagta tcacatgttg agatatggaa ctacatttga |
| 39481 | aattcctgta gaaatatgac tggcatgtta gtacagtaga attataaata tgtatttgtt |
| 39541 | ttgttggaaa agtatgaata tagaaattaa tcataaacat ctagctctta ttgacttaaa |
| 39601 | taaatatgct aaatgaaata tgattacatt gaagagattt tcctcaactt ttaaaggtca |
| 39661 | aagcctcagt tatttagtat tattttttaa tttagaaagt attgagcaca taggtgaagg |
| 39721 | ctagcttaaa ctagatgtat ccaaacctga atattaggat taacaatgga tttaaaatgt |
| 39781 | aggttctttc tgataccaca tcgacaacta caagctgttt tgacaactgc tataatggac |
| 39841 | ttttcctgtg tttctactaa atgcaacaaa tgtgaccttc ttttttctga ttagcattgg |
| 39901 | gggtgagatg caggaatctg tgtcagggag cttacagtcc agaatttgtt gagtgccagc |
| 39961 | tcgtattcaa gtgttgttct ggatacatca aatacaagac attatttaat ctatagtcat |
| 40021 | atgtagattg ttgtcatttc atgtgtgccg aataatatta actccggtgg atgataaact |
| 40081 | aaggattaga gacaaggcag tgggagttgt caagtcacat ggtcagtaaa ttctggaatg |
| 40141 | tgatttgtgt caaggtctgc ctgactccat agatagcgct cttcctacca taccctgctt |
| 40201 | acaaaaacaa gatttaggtc ctgatttatt cattgatttt tgSaatcatt gtagataaaa |
| 40261 | agcatagtta agtaaaggtg tgtaggcaag aatgaatgag taccatagaa agtttaggaa |
| 40321 | aagtgagaaa aaataatgtc ttcaccatat tatctgactt gagtaagaaa gctatgtgaa |
| 40381 | tcaatgtaag aattttggag tcatacaaac atggttttaa atacagcttc ttctatttat |
| 40441 | aagctgtata tccatggata tgttttttaa tctccttaat cctcattttc atctataaaa |
| 40501 | tgaggtgaat aataatcacc tcacaggata ttttcaaaat gaggtaaatt aaatactaag |
| 40561 | tacagggccc aacccatagt aagcattgag tagttggaaa tttctattat catgattgat |
| 40621 | tggataaata aggacagttt gaaggataga agacttatca taaagtgaga gtaacaaata |
| 40681 | gcttattaaa agttagaaag agtgaaagtt tttatgaaga ggatgatttc aaaattaaaa |
| 40741 | atgtaaagta ttagcacaat ttaggaagat gccgaaatct gaaatatagc ttttaatgat |
| 40801 | tttgattgaa agggatttga agaattttga gactagagta ttagaaggct aatcaaaatg |
| 40861 | gctatcaaat ttactgagga taatggtgag acatttttgg agagaaagac caatagcctg |
| 40921 | atgctgatga cattaaagaa gctacagaat gtgatgataa ggaagctgag gagaggatta |
| 40981 | tccagtcYag aatgatgttt ctgcaagaac taggggcctc ttatgttaat ggagatggag |
| 41041 | actgtagtac ttgctaaaat tttcgattcc atgactccac cctttatcat gtgaatcaga |
| 41101 | atctctggag atgtcttcca ataacctgaa tgtttaacaa gctccccaga taaaactatg |
| 41161 | cataataaaa atgatacttc taaagtttga aaactatact tctagaaaga tatccaaatt |
| 41221 | gcactgagaa ctctttgcct gaggtgatat tagtgaaggg atgaggaata tgggaacaga |
| 41281 | aagtaagcaa ctggtgtggt ctaaagagag tcaagagaat caaaaacaag caagcaaaaa |
| 41341 | cagaataatc tgcttttgtc tgatttcgtc aaggtagaga gcataatctg ggagaaaaaa |
| 41401 | gtgaattgct tccagggcag agttctaagg attttctttt ctttggaata aaatgatgga |
| 41461 | tgaaagaaag aaaagaatag tgtgctgaga atgtaaattg tcttactcac tcattcacaa |
| 41521 | aattttaaag gtagttggga agatcaaacc taacactgta tgaaatgaca tttaaatgag |
| 41581 | tatctagagc attataggtg ctaaataaat gttagaatct cattactcac ccttattcat |
| 41641 | ttgtcttata aagagcattc ctgtgataaa caattatgtt atgttgcctg acatcagcct |
| 41701 | ttctctaaac atttagatag caaggagact gtatacaatg cccaaaaagg aacatttaaa |
| 41761 | ctagcatgaa acgtaaagga tcactgatgt aaacagtttt tcatttgcta accaatttct |
| 41821 | ttatggcatt cccggtagat gctaatttca ttcattataa gagatttttc accacataat |
| 41881 | acaccattgc tctatcagct cgaatacaca atcttcctgg catagctttt acccacaaca |
| 41941 | taataaacag caatatagtg ctaaatgtaa gacagaaaga gctaaatagt tatcctgctt |
| 42001 | atgccaattt atagcagctt tggaatggga gcttgatgtg ggagagaggg gaaatggaaa |
| 42061 | tggaatgttc tttcatcttc tgctataaag ttgcattcac tcataacttt ggacatttgt |
| 42121 | ttaggtaatg gattggtgac aggaagaaat gaagaaaaag agaaggcagg aggagaaaga |
| 42181 | aagacagaaa gggcacctta gagtaccatt gtctgattat atcctcctct gtgctgcctc |
| 42241 | taatctttat caacataaca gcacttacta catggtgttg tttttatcca tttataggtc |
| 42301 | tatctcccca ctagtatatg tgttcattaa aggcagcttt ttttcttatt tagccttatt |
| 42361 | tatatttgta cttatttgta aaacaaacta ataacttagg gcttaccata tgccagaaat |
| 42421 | gcttctacgt atctttttgt gcatcagctt atataatcct aatggtgtat atgtaaagtg |
| 42481 | ctttagcaca gtgtctggca catggcaaac cctcaaaaat gttagttaac acttaatcat |
| 42541 | tattgtttat gttaacaacc ctctgaagta agtgttgcaa ttgccatgtt atacctggag |
| 42601 | aaaatgaggc taatcaaatt aaactggctt gaactagatt aaacaatttg tatgatttac |
| 42661 | ctaggattgc atccatcttt atttgatttc aaaatgtatc atattcataa tcttggttat |
| 42721 | gaatattata caaccttttt gaaaaaacta tggaagtgat ggctctccac atgtgacttc |
| 42781 | aaatgttttc aattaaatta ttttgtttca atagttttta tcataataaa ttttaagact |
| 42841 | atgttttgct aatttcattg tcaagccaaa tttgtatctg cttaatctat aatatgaaat |
| 42901 | ttaaaaccat gagaatactc cagaaaagtt ataatttttc tttttctttt tctttttttt |
| 42961 | tttattatac tttaagtttt agggtacatg tgcacaacat gcaggttagt tacatatgta |
| 43021 | tacatgtgcc atgttggtgt gctgcaccca ttaactcgtc atttaacatt aggtatatct |
| 43081 | cctaatgcta tccctccccc tccccacacc ccacaacagg cccccgtgtg tgatgttccc |
| 43141 | cttcctgtgt ccatgtactc aattcccacc tgtgagtgag gacatgcggt gtttggtttt |
| 43201 | ttgtccttgc aatagtttgc tgagaatgat ggtttccagc ttcaaccatg tccctacaaa |
| 43261 | ggacatgaag tcatcatttt ttatggctgc atagtattcc atggtgtata tgtgccacat |
| 43321 | tttcttaatc cagtctatca ttgttggaca tttgggttgg tgcttagttc caagtctttg |
| 43381 | ctattgtgaa tagtgctgca ataaacatac atgtgcatgt gactttatag cagcatgttt |
| 43441 | tataatcctt tgggtatata cccagtaatg ggatggctga gtcaaatggt atttctagtt |
| 43501 | ctagatccct gaggaatcat cacactgact tccacaatgg ttgaactagt ttacagtccc |
| 43561 | accaatggtg taaaagtgtt cctatttctt cacatcctct ccagcacctg ttgtttcctg |
| 43621 | actttttaat gactgccatt ctaactggtg tgagatggta tctcattgtg gttttgattt |
| 43681 | gcatttctct gatggccagt gatgatgagc attttttcat gtgtcttttg gctgcataaa |
| 43741 | tgtcttcttt tgagaagtat ctgttcatat cctttgccca cttgttgatg gggttgtttt |
| 43801 | tttcttgtaa atttgtttga gttcattgta gattctagat attagccctt tgtcagatga |
| 43861 | gtagattgca aaaattttct cccattctgt aggttgcctg ttcactctga tggtagtttg |
| 43921 | ttttgctgtg cagaagctct ttagtttaat tagatcccat ttgtcaattt tggcttttgt |
| 43981 | tgccattgct tttggtgttt tagacatgaa gttcttttcc tttttttttt ttttttcaga |
| 44041 | cggagtctcg ctctgtcgcc caggYtggag tgcagtggYg cgatctaggc tcactgcaag |
| 44101 | ctccgcctcc tggattcacg ccattctcct gcctcagcct cccaagtagc tgggactaca |
| 44161 | ggcacccacc accacacctg gctaattttt tgtattttta gtagagacgg ggtttcactg |
| 44221 | tgttagccag gatggtctcg atctcctgac ctcgtgatct gcccaccttg gcctcccaaa |
| 44281 | gtgctgggat tacaagcatg agccaccgcg cctgacccag aaaagttgta atttttaaac |
| 44341 | actatgacat caacataatc caaaacaact tttttggcat tgagtagata catcttggtc |
| 44401 | agtaattaca gtgcctcact ttaaaattga ctaggtttaa ttatgtacaa aaaagtggtc |
| 44461 | aaaagaatgc tctgttccat taaatagtca caatgtatag aggaaataaa aaaatgtttt |
| 44521 | ctgagataca cctttttgta ccttttctct ctgaaaagtt catctgaggc ctgttgcatt |
| 44581 | ccactatact catccttaaa acaaaatatc ttctaaaatt attgcactat agccaactat |
| 44641 | gaaatttggc tattataaga tgaatagcac tggaaacttt tcaagctgat cttatttaag |
| 44701 | aggagtagac cctttctgtc aaactactaa gtaggatatt gaaggggaaa tgtcatctta |
| 44761 | taattaacat agaatgaaaa tattatttgg catgtttggt agtgtgtttt gccaattagc |
| 44821 | cacaaaacaa aaaataagaa tcgtcaacaa caaaaaaaac cctcaaatcc aaaaggaaac |
| 44881 | aaagaattca taatgcccta agtcagtcta tttctctaat tcttttcttg tttgtccagg |
| 44941 | taaaactagt taagaaccat aattttacag gaatatcttg tgaaatcatt gagaagcaac |
| 45001 | ttgacctcaa tctctctctg cctctctctc tctatctctc cctattgttc tccctctcct |
| 45061 | tctccatctg tagctcgcta tctttctatc attttctctc ccccttaccc ttcatctctt |
| 45121 | cttcttttcc atcctgtacc tccatgattc cttctttcct atccctgttt tgaagagcaa |
| 45181 | aacaatacaa aaacagtaaa aataatatca atgccttaat agaaataact attatagcag |
| 45241 | ctaatattta taaaagcttt acaagctcta acttatttac acttcacaac aatcttataa |
| 45301 | gataacaata attatgccaa ttttatcata tctgtcactc taactataat tgtttattgt |
| 45361 | atgaatggct gaataaatga atatacttca cagtattagt ttgcagattt tatttgcaag |
| 45421 | atatcaaaca aatatatttc tggatatttc ttggagatat atattagaaa cagatcttat |
| 45481 | tgatatagtc tgcttctatt gtttggaggg catggggcag ggaagaatct gcaatgaata |
| 45541 | ggcaagaagt tgcaactagc catttgaaac ttaaatgatc aatttgggaa actagacaac |
| 45601 | ccataaaaaa tccttacctt ctttctacat tcttggcttt tccctaagtt ttaaccaatg |
| 45661 | aattagcttt aaaatatatc tacctgtctg aataccttgg agagcagtgt gttacttttt |
| 45721 | ggccaactgt tcaaatgtta cattttaata agttcccact tgtggtcatg ttagatatct |
| 45781 | tcttgaaagg atgaggtcat gaaaaagaag tttaattgga tcacaaaact gcacactttt |
| 45841 | ctaggtMaaa ggtaaaggct aggaaaagaa ttcattatgc tacaagaaag aaagtaacct |
| 45901 | ttgggtacag aactatcatt tttaatgatt aagaacagca tttactatta gaaatatatt |
| 45961 | cataatagtg gctttatttt aattaggaac ttcattcatg ccccagcatg cactatgaac |
| 46021 | tctttaggat atgttaagtt acagtgtggc atggcaggtg atgaaaaatg taaggggaaa |
| 46081 | ttaaaaaata aattgagctt agggtattag aatacataag tgatgctcaa ttggcagtga |
| 46141 | atttgacata cacagttact ttagaagagg caaaagaaga gaaaatggaa tttctStcct |
| 46201 | aggaatgatt gcttaaatat cttgaatatt ttaattacat gtttcaaaac agaaacgttt |
| 46261 | gactagaagt taatgggaag tgagaattag gattcaaata tagcccccaa agtaactctc |
| 46321 | attttaaaaa tgtacattcc agttattaaa gttgagctaa aaattgtact attaaaaatc |
| 46381 | acacttgact gccaaatatt ataggagtct attctctgtt gattcatttt gttgacatta |
| 46441 | agagaagctg catgagttca aggaggatat aattcaagag gagaacaaaa gcaacaagca |
| 46501 | gcggctcagt ttaaagcaag gaactcttta ggactctata ctgagtcact tatagatggt |
| 46561 | aaagtctagt tcaagattca gttagctgag gaacaattgt tgaataaaga gtcattaatt |
| 46621 | ttgctttcaa ttttatttat gtaaatgata aacattcatc tagtgccttt cttttttaaa |
| 46681 | aataaagcaa tggaccctgg catcttgtat tttctgttta ttccatgttc atttgttttg |
| 46741 | caaagctgat cagagtatta gatatcagga cgaacacagc agtcaatttc aggattttat |
| 46801 | ctagtatgtg tatttccagg actgatgcat tcaaaggatc cgggatttag tgagtagttt |
| 46861 | atatcaagga gactcttgaa aacaacctga agtgagcctt aagagcagaa gtgaggcgta |
| 46921 | tagtaggtgt ttagYaggtg ttgtgccaga cttattgaat ggatcatcaa atttagatca |
| 46981 | cagaataaga aagcaattga gtttaccctc tcaaataaag tgattttcat ctcaacttta |
| 47041 | actttttacc ctcctttgca tttttcattc tttgagtctc tccactgttg aaaatgtagt |
| 47101 | aatattaaaa gttYgagaga tcaagtttaa ataagagaat ctcatgtgca gttctgtgtt |
| 47161 | ctatgtataa tttaaacttt tcagagcacc atgatgaata aaaattatgt atacatagaa |
| 47221 | aacagacatc tgaaagtaaa ctcatttcac tcacacacaa aatctcacat aatatctacg |
| 47281 | gagaaattca ggaaaatcct tccaaaaaga aaggtggggg gaagtagcat ctgataagat |
| 47341 | attggaaaaa agaatataat cccaatgtat tacaacttct gcctgaagca aagttctctt |
| 47401 | gtactctgac tgggtcagat ttcacagata aaggtctaaa ctctggtagg gtactctgat |
| 47461 | atagtccctg gtgagaagtg gagtgattct attaatgaaa tccttgtctt tgtctgaaca |
| 47521 | aagaccattg ccccaggagg gcccttgaga gccacaggta gcctttcaaa aggaatgtaa |
| 47581 | agctaaaatt ctcgacctct tgaggtccac ggtgctccca ggacattttt cagtagaaat |
| 47641 | gagatcatat ccttggcttc acaggtcaat ttcaagctgc ctcctgccta tttaaatctc |
| 47701 | tagcagcttt agagagatag catagtattt ggtgagtgag ccaaagtttc atatcacttg |
| 47761 | taaagtactt acacttgaaa ggtactataa attcagaaca ttatcatcaa ggcatccagt |
| 47821 | taagaacatc aaaagctatt ggaattttaa aatagaaaaa gaaaaagtgc tattctccag |
| 47881 | ggtactgcta aaaagagaaa ggctctaaat ccagggtcat tgaagaacct cagtgaaatg |
| 47941 | ctttaattag aaatggcaag ccttgtggtt tattttcaca gccatgggaa tgcagtattc |
| 48001 | ccaaactttt tttaccctcc tacccttctt gctttctgtg ttcccctgca gcaaaacaca |
| 48061 | tagacatgct tactaatact gggaaacaca cctcaacttg ctttggaaag ccttttgtgg |
| 48121 | aagtacaata aactccagtg tttataaatg ttaataatat aagaaggggg ttcagaattt |
| 48181 | catgaagata gttaagaatt taactcactt ctatcagcaa tttccctcag ctcaaaaaat |
| 48241 | actgcaatca aaatcatttc tgatgaaaat gccactgttt taaatgtcta cttataataa |
| 48301 | aatgagccta cttataaatg atagagctat ttatggtatg gcagtaactt cacaaaataa |
| 48361 | catgattctt agaaatgtgt ggtctttggc ccagcctggc ttttaaaccc tcaaagccaa |
| 48421 | atatattatg gatgtcttgg aagaaagtaa actaggtact tggaacacac ttggaaatgc |
| 48481 | aatcacaggt cacagaggtc ttctttgtgg ttatgacttc aaatagtaga ggtattcagc |
| 48541 | ggagctgaac tctgcctttc aagctaggta gattggattt ttctcttaaa gtttgtaggg |
| 48601 | aatcagtgtc cataaataaa aagggaatga aaactgtgac tggaaactga gaatgttgat |
| 48661 | ggacaRtgat tcagctctga gctgcttcgg cttttcttcc ctggtctttc tattgtccag |
| 48721 | caaatgaaac acactataaa accacaagaa aaaaaaacaa agtattggac tgggggagga |
| 48781 | gagagaggcc ttgtgatcct aagaaaaact atgtaaccag cagcctaagc aaaacaggga |
| 48841 | aggatgcaca ttcttctgag ctaatcataa tgaacttaat ggcaaatact gaaagaagaa |
| 48901 | aaagctttgg agctcttctg tagcaataaa cacctgagaa aatgcagttt ttcaatatgt |
| 48961 | atgtccaata ttgtatagca atacctgtta tgttttgctg ttttcaaagt ggaaaaaatc |
| 49021 | atttcatcag tgtggtaatc cttgaaagcc tcattcttat agttatacca ggattcaaat |
| 49081 | cctatcatga ctctgccttt taagtagttt gttctttttt tccaacttgg gcaagttatt |
| 49141 | taacctattt aagcttcaat ttcttcaatt ataaaatgag taaaataata tatgtgacaa |
| 49201 | tatgcatgac tttgtcctgt tgtgtccgga attggtgggt tcttggtctc actgacttca |
| 49261 | agaatgaagc cgcggaccct cgcagtgagt gttcagttct taaagatggt gtgtccgggg |
| 49321 | tttgttcctt ctgatgtttg gacgtgttcc gagtttcttc cttctggtgg gtttgtggtc |
| 49381 | tgctggcttc aggagtgaag ccgccgacct tcgcagtgag tgttacagct cttaccRcgg |
| 49441 | cgcatctgga gttgtttgtt cctcccgtcc ggagttgttc attcctccca gtgggtttgt |
| 49501 | gatcttgctg gcttcaggag tgaagctgca ggccttcatg gtgagtgtta cagctYataa |
| 49561 | aggcagtgcg gacccaaaga gtgagaagca gcaagattta ttgcaaaaag cgaaagaaca |
| 49621 | aagctcccac ggtatgaaag gggacacaag caagttgccg atggtggctg gggcagcctg |
| 49681 | cttttattcc cttatttggc cccacccaca tcctgctgat tggtccgttt tacagagagc |
| 49741 | tgattggtcc gttttgacag ggtactgatt ggtgcattta caatccctga gctagacaca |
| 49801 | gagtgctgat tggtgcattt acaatcctct agctagacat aaaagttctc caagtcccca |
| 49861 | ccagattagc tagatacaga gtgctgattg gtgcatccac aaaccccaag acagacacag |
| 49921 | agtgctgatt ggtgcattta caatcctcca gctagacata aaagttctcc aagtccccac |
| 49981 | ccaactcagg agcccagctg actttgccta gtagatccca cgcaggggcc gcaggYggag |
| 50041 | ccgcctgcca gtcccacgct gcgtgcctgc actcctcagc ccttgggtgg tggatgggac |
| 50101 | tgggcaccgt ggagcagggg gtggtgcccg tcggggtggc tcaggttgcc tgggagccca |
| 50161 | cgggggtagg ggggtggagg ggccttgggc atggcgggct gcaggtcctg agccctgccc |
| 50221 | catggggagg cagctgaggc ctggcaagaa ttcaagtgca gcgcaggtgg gctggcagtg |
| 50281 | ctgggggacc cggtgcaccc tctgcagctt ctggcccagg tgctaagcct ctcactgccc |
| 50341 | agggctggtg gcaccagcct gcggcgcaga gtgtaggacc cactaagccc acgcccaccc |
| 50401 | agaactcacg ctggcctgcg agcactgtgc acagccccag ttcctgccca ctccactccc |
| 50461 | tccacacctc cccacaagca gagggagccg gctctggcct cggccagtcc agagaggggc |
| 50521 | tcccacagtg cagtgtcagg ctgaagggct cctcaagtgc agccagagtt ggcaccgtgg |
| 50581 | cctgaggagg tgccgagagc gagcgagggc tgctagcacg ttgScacctc tcactgtggt |
| 50641 | gaggaataag taagattaag cgcttactac agtatctggc atgtgatatg caatcaaaaa |
| 50701 | atgattattg cactgttgtt accattgcca acttcactgt gaatatacaa ctttcttttt |
| 50761 | gaagtataac tcttgggata attttagcat catttttgtt gaggcttaga aaattacacc |
| 50821 | tcaaaatgaa ggccttacaa gcagccttag aagccaaact ttctctctga ccttctcctg |
| 50881 | tcctcttgtc tctggcccct cattttcccc agagggtagc catagagact agaatccctc |
| 50941 | tcccctcaag gcaggtcata gaaactagaa cttacagagt tattcctgta tctttgagtc |
| 51001 | ttcattctga aggctcttat gtcttgtatc tcttgtgcct tttaaaacta tgatcaaata |
| 51061 | tattttatgc cttttcttct attaatgtcc cttttgtcag ttgatttcag caaaccttca |
| 51121 | gagaggcaaa ggggaaggtc tttcttggcc cctataattt catgccatat ttgacttttc |
| 51181 | ttggcctttt atatttgaca acaattttct gatttctgat ttcctgattt acataataat |
| 51241 | caattttata tatgtattat gtaagtattc cctactaaca gtgactctca ggttcctaga |
| 51301 | tgatgtctga ccttggtttt acttgtcttt aaaaagtgcc actaaattcg ctattgtgac |
| 51361 | atctagtagc tgtcagtgat tctggagcaa ctattggaat catagtgtta tttctggtga |
| 51421 | catcattaca atgtaaaact acagaatttc ctctcaattt taattcttca tatagagggt |
| 51481 | tcaagattct ttctgtaagt gcaaaataat cttagaaaaa atacatttct gaattgatag |
| 51541 | taacaggatt tgcatttata attttcaaat gaaagacaat atgctaaaag atgaattttg |
| 51601 | tgatcatctg taacatgttt ttttcagctc tgatgaagcc ttcagtaaag tcaatttaaa |
| 51661 | ttaccgcact gaaaatgggc tgtctctact tcatttatgt tgcatttgtg gaggtgagta |
| 51721 | cttgaaactt agtacttctt aaactggtta tatgtgtata tcgtcagtga cttgagctgc |
| 51781 | ttaatagtct gcaaaagaat tctattcata ctcaattttc ccttctgctt tgccttttcc |
| 51841 | aaccttgata atctcagttt aggcagtaac tatcaggcag aatattccaa ttgatttagc |
| 51901 | tttaaatata agcttcattg tgttgctacc ggaaaccaag tccctcagtt tgcttggaaa |
| 51961 | tcaaaaagca cttttcaata ttgggcatgc cagtcaggga ttaacttaaa ttcagaagca |
| 52021 | cttaaagatt ccccaaaata tcatcaagca gttgcttcac aRtattcaac aagagtatgt |
| 52081 | ggaaaagtga taaatgccaa agataagatc taaaagtgaa gattgatcga agtgaagatt |
| 52141 | aagagtttaa tttggcctac actttagttt gttttgttta tcaatgtatc tcaagtaatt |
| 52201 | tgaatggtgc ctggcctgta atagccactc agtaaagtta ttgaattaat gaatgaacca |
| 52261 | ttaatattta ttttgttaat ccaaactaaa ttcattgagc ttaacttctt aaaatggaac |
| 52321 | agaaatgtgt aagtgtcaac tctaaccata ttccttaacc atttctccca aacaaaccaa |
| 52381 | acagactctg cttaacagat tgttctctca acttcaaacg tgccttccca accttggtta |
| 52441 | tatccttcat atttctttga tctaaataag ctctactgtt aataatgttc agataaatcc |
| 52501 | tctacaatta gattttttaa tttaagcttt ttcactaata gtttattatg gtgaattctc |
| 52561 | ttatgtgttt atgtttcaaa accatctgat gatgagggta ctaaataaaa aatctaagac |
| 52621 | agagtcaaaa tacacagcat ttgaataaag ttttataaat ggaaaagaaa tatcccctag |
| 52681 | taagtaaggt tgtcaattaa tagaaaactt tacaaatggc ctgcagaacg cagcttgaca |
| 52741 | tttttataca gctgtatggc atttatcatt aggtcaaaaa gagtctcaaa ctcaccccat |
| 52801 | ccccacaatg atttagcctt ttttcatttt tctctttaag gcaagaaatc acatattcga |
| 52861 | actcttatgt tgaaagggct ccgcccatct cgactgacaa gaaatggatt tacagccttg |
| 52921 | catttagcag tttacaaggt aggacacttt aattcccata aacactgcat tggaactaag |
| 52981 | gatagtgtgt atctttaggc tttttttttt ttttttcaaa ttagtaatca tggaaaattt |
| 53041 | tcagtgacca gaggcgttac attactaaag gacttctttc tctttattga gcagatacct |
| 53101 | gctgcatact agacacatac tctttttaat gctttctgta tttaactcat ttaattctca |
| 53161 | caagaacccc atgagtgaat atgattatta tccccaaggt tacatggtta gccaggggtt |
| 53221 | agccaggatt taagacaagg cagtctagtt ctgttgccaa agctcctcat cagaacacca |
| 53281 | tgatgcctct gtgcagagag agatgaggtt gtatttaaag acaaaagaag cacactgaag |
| 53341 | ctgtatttct ttactgcaaa tttttcttaa aaaacctttt tgctttagct gtttattttc |
| 53401 | ttatattagc ccctggctcc aacctaatct cagcacctga tagaataata aatataatag |
| 53461 | tggattggac aaactgggat acttagtgtt ggaattgaac cttgagattt tcataaatat |
| 53521 | aaatttataa aacatacaaa ttatattatg cattgatcat tttataatta taataacaat |
| 53581 | ttctactttg tatttattat gagctaaata tcatactaag tttaatgtat tttagtcatt |
| 53641 | aaaataactg catattagga tttactttac agaaaatgca attaaagact aaagtagata |
| 53701 | cctggacaat attagaatag tgattaaagt caagtacagt tggctcatta ctcccagaat |
| 53761 | cttatttgta tattgcgtat tttgttcttt ctcattttca agcgagtctg tgacagaatt |
| 53821 | ttatttcagt tttgtgtgtg cacgtgtggg aggagggaca actttttgct tgtattagga |
| 53881 | accccctgca ctatctccct cccccaactg ctagagagtt tgtcattggg agggaagagg |
| 53941 | accttatttt cccatgatct agcatcacaa attcctgtaa ttcatagcgc aaatactcta |
| 54001 | aagtttcatc tcttttcatg cccagtcaat gcctagtcaa ttcttctaat tccttctggg |
| 54061 | agaaagcctc agttaagtgc taaattgtct ttagtacctg ttaattatcc tgctaaacta |
| 54121 | tgaaacattc tgagtttcca gtgtagaaaa cagcatctga tgttgacttt tcttctgaaa |
| 54181 | ggcaggaggt gaaagtagaa gagctccaag gaacaggaag gtagtgaatg atagtcatgg |
| 54241 | cactgtatga aaccatgaat tctgaacctc accgtctggg agattttttc tgtattagtt |
| 54301 | cattttgcaa agatttagca tgacattggc acacaaatgg gttctgaatc atttagtcct |
| 54361 | acaatattta gagaatatat ttcttcaacc tctgtttata ataattaaat ttccactttt |
| 54421 | agagttttct ttgctctatt tttatgtttt tttctatttt agagaagggt gcataaacta |
| 54481 | taactttttc atagcaagtt taaatgaaaa tttagattct tgggaaacaa agcaggcaaa |
| 54541 | aatattaaaa ggccataaga aaatgcatag ttattttgca aatttcttaa caaaagtgaa |
| 54601 | aaacaaatta aagttactat ttcattttaa acacaaaaat aatataaagt agttgtgtgt |
| 54661 | ccagaattgg tggattcttg gtctcactca cttcaagaat gaagccaggg accctcgcgg |
| 54721 | tgagtattac agttcttaaa ggcggcatgt ccagagtttg ttccttctga tgtttggatg |
| 54781 | tgtttggagt ttcttctttc tggtgggttc gtggtctcgc tggctatgga gtgaagctgc |
| 54841 | agaccctcgc agtgagtgtt acagctctta aggcggcacg tctggagttg ttcattcctc |
| 54901 | cccgtgggtt cgtggtctcg ctggcttcag gagtgaagct gcagaccttc gcggtgagtg |
| 54961 | ttacagctca taaaggcagt gtggacccaa agagtgagca gtagcaagat ttattgcaaa |
| 55021 | gagctgaaca acaaagcttc caaagtgtgg aggggcaccc gagtgggttg ccactgcttg |
| 55081 | ttcaggcagc ctgcttttat tctcttatct ggcccccacc cacatcctgc tgactggtcc |
| 55141 | attttacaga gagctgattg gtccgtttta cagagagctg attggtccat tttgacaRgg |
| 55201 | tgctgattgg tgcatttaca atccctgagc tagacacaaa agttctccac atccccacta |
| 55261 | gattagctag atacagagtg ccaattggtg catccacaaa ccctgagcta gacacagggt |
| 55321 | gctgatggtg tgtttacaaa cgttgagcta gatacagagt gctgattggc atatttacaa |
| 55381 | tccctcagct agacacaaag tttctccaag tccccactag actcaggagc ccagctggct |
| 55441 | tcacccagtg gatcccgcac aggggccgca ggtggagctg cctgccagtc ccacgccatg |
| 55501 | tgcctgcact cctcagccct tgggcagtca atgggatggg gcactgtgga gcagggggca |
| 55561 | gcacgctttg ggtaagctcg gccatgcaag agcccatggc agggggtcgg ggggaagctc |
| 55621 | aggcatggcg ggctgcaggt cctgagccct gccgcacggg gaggcagcta tggcctgctg |
| 55681 | agaaatggag cacagcgcca gtgggccggc actgctgggg gacccggagc accctccgca |
| 55741 | gctgctggcc cgggtgctaa tcccctcact gcccggagcc ggcaggactg gccggccgct |
| 55801 | cggagtgcgg gcccgccaag cccacgccca cgcccaccca gaactctagc tgggccgcaa |
| 55861 | gccccgccgt gcggccccag ttcctgcccg tgcctctccc tccacacctc cccgcaggcc |
| 55921 | gagggagcca gctccagcct cggccatccc aggaaggggc tcccacagtg cagcagcggg |
| 55981 | ctgaagggct cctcaagtgc agccagagtg ggcgccaagg ccgagaaggc accaagagcg |
| 56041 | agcgagggct gcaagagctg ccagcacgct gtcacctctt agttgttcac actaccaggc |
| 56101 | attaaaccta gtaaccatta aatgcattaa acctacttct aattatccct tgacagaact |
| 56161 | actccagtaa gtatataatt cattttgggc tccagttccc ttcccccata tagtattaat |
| 56221 | aactgtttaa ttttaatatt atttcaaact tacaaagaca attgcaagaa tagtacaaga |
| 56281 | aactgcttat ccaggttcat taactgtttg cattttgtcc catttccatt attttttttW |
| 56341 | agaaccattt gacagtaaat tgtataaaac atgccctgtt gccacttcag tcttcatttc |
| 56401 | ctaagaacaa gaaaattcac ttacataact ccaatgcaat tataaaatta aaaagaacat |
| 56461 | tgagatgata ttaacattga taagatatga taatctatag ttcaaattca agttttatca |
| 56521 | attgtcccaa ttgtgtcatt tatagattat attttcttgt ccaggatagg attcaatcca |
| 56581 | caatcatgca ttttcagttt tcttatttct gtaatctccc ttactctgaa agaatttctc |
| 56641 | acccgatcct tgtatttttt tatcttaata tttttcatgc ataccagtta tttatacata |
| 56701 | tttctttgaa tttttttggt attgcaatcg aattttattt tgttcataac actcacaaaa |
| 56761 | tgaataatgt aactctccca tccttttgtt cactacatca aatagctctt tcaattatac |
| 56821 | accctggcat gctgggcctg actcattgtt agaggaaagg ggtcccgatc caggcctcag |
| 56881 | gagagggttc ttggatctcg cgcaagaaag aattcagggc gagtccacag tgcaaagtaa |
| 56941 | aagcaagttt attatgaaag taaagtggtg aaaggacagc tactccatag acagggtagg |
| 57001 | atgttcctga aagtaagagg aggagcacat ccaccctagg tacaatgctc atatacatgg |
| 57061 | ggagatgtgt tctgctgcaa gggtttgtga taacatatta attttcttaa ttgctatatt |
| 57121 | ttgcaagaat cgatattatt atctttaaag caaaattagg aatgcctttg ttctacagat |
| 57181 | atcaggatat ctggacactc ccaagtctgg gtctgttcag taaacattat gaacttgttc |
| 57241 | tttaactgta aacatctaga ggttaaaaat gcctaacttt tgggaggccg aggcgggcgg |
| 57301 | atcacgaggt caggagatcg agaccatccc ggctaaaatg gtgaaacccc gtctctacta |
| 57361 | aaaatacaaa aaattagccg ggcgtagtgg cgggcgcctg tagtcccagc tacttgggag |
| 57421 | gctgaggcgg gagaatggcg tgaacccggg aggcggagct tgcagtgagc cgagatcccg |
| 57481 | ccactgcact ccagcctggg cgacagagcg agactccgtc tcaaaaaaaa aaaaaaaaaa |
| 57541 | aatgcctaac tttctgagaa tgtaaccccg taagtctcag cctcattttc ctattttcct |
| 57601 | agccctcact caaaatggag ttgttcactc tggttcgaac gcctctgaca tcatgactgt |
| 57661 | atattttcta atcaatagag taatctctgc tttcattaat aaaagtgaag cagatattta |
| 57721 | gctatttaat accacatgaa tgattaatcc tcatacatct ttttgtttta gtttgttgag |
| 57781 | tttttcacag tgtttagttt tattgaggtt taatatatac caaatacaat tatcaatttt |
| 57841 | aagtctacaa ttgttagctt ttgaaaatgt atgtaatcat gtaaccatca tcacagtcat |
| 57901 | gttataggac atttctacta taccaaaaag ttccctcatg ccattttgca gtcagtctcc |
| 57961 | ttctcacctg gtctctggaa acaagtgttt tctgtcacta tagttttctc ttttctagaa |
| 58021 | ttttaaataa atttattaca tccagtatgt tttcttttct atatggtttc ttgcacttat |
| 58081 | cataaggcta ttgggattca cccatgttgt atattctgta attaatgctt ttcttgtaga |
| 58141 | attgtattcc cttatatgga tataccacaa tttaccattt acctgctgat ggacattttc |
| 58201 | ccagattttg accattataa acaaagctgt tctgaacact cacatacagg tatttgtttg |
| 58261 | aacatatgtt tttatttctc ctgggaaata cctaaaagtg gaattgctga gtcacataat |
| 58321 | aagtatatgc ttaattttat gagaaactaa caaacttttt aacaaactag caattccttc |
| 58381 | ctacattccc atcagcaagg tatgagagct gtagtttttt cacatccttt ccggcacttg |
| 58441 | gtgtggtcag tctttttttt tttttttttt tttttttttt tttttggcta ttctattgga |
| 58501 | tgaattttag tatttcatta taattttaat ttgcatttcc ctgaagacta aagaggttga |
| 58561 | ataggttttt atgtgcttat taggcatcct ttatttttta atgtagtgtc tgttaaatct |
| 58621 | ttttgccatt tgtaattttg tttattattt tcttgaattg tgagggaact ttatctattc |
| 58681 | taatacaatt cttttcttga atatactttt gcaaaaattt ctctcagtgt ctgacttgcc |
| 58741 | ttttcatttt cataacagtg tcatttgaag agtaaaaggt ttttgcatta tttttaaact |
| 58801 | tatgactgta gtgattacca tacagttata gattctcaac atttcatagt ccactccctg |
| 58861 | caaatgttga tctacttcac ttaaaatata agaaccttcc agtagtatag tttcatttta |
| 58921 | tcttcattct ttgtgccatt gttgccatat acattgcatc tatacatgat aagaacctaa |
| 58981 | atatattatt agaatttttg tgttaaacat ttatatcttt taaagaaatt aataagagag |
| 59041 | aaaatatgct tttcggaagt ttgattataa taatcctaca tatggttttc tgtatttaaa |
| 59101 | actctttgag attcactgag tttttatatc tgtaaattta tggtttcatc aaattcagaa |
| 59161 | attttgacca ttagtttttc aaatacatat gttagattgt ttgataattt tccacatatc |
| 59221 | actgagtctg ctttttttct tccttcttct ttctttctgt tctccaaatt agattatttc |
| 59281 | tacttatttg aattcaagtt cacaaaccac ttctctgcca tctcccatct gataacaatc |
| 59341 | caccagtgaa ttattcattt tggttgttta cttttccgtt ctaggatttt tatttattaa |
| 59401 | gattaattaa gatataattt caatttctct gctatgatga cacatctgtt atttcatttt |
| 59461 | tccatatttt ccttcaaggt tttgaaccta ttatttgtga ctactttaaa tgtccaatct |
| 59521 | gctgttttca acatctagat tagctcagta tttgtttcta tttcatgttg actacagRgc |
| 59581 | acatttttct gtttcttcat atatcaatta attttttatt gaaagctaga aattatgtat |
| 59641 | tatgtgttat aggaaatctt tatcatgtca tcttccttta aagaatgttg agtttttttt |
| 59701 | atgtttgttt ttctttcatt tttggcgggt agttacactt ctggtggata catttcaatc |
| 59761 | aaatcagtct tgatttgatt ttttgttagg ctgaaggtat ttcagtttga acttagcctc |
| 59821 | ttggcttacc tctttttttt tttttttttt tttttgaggc ggagtctcgc tctgtcaccc |
| 59881 | aggctggagt gcagtggtgc gatcttggct cactgcaagc tccgcctccc ggttcaagcc |
| 59941 | attcttctgc ctcagcctcc ggagtagctg gggctacagg cgcccgccac cacgcccggc |
| 60001 | taattttttg tatttttagt agagacgggg tttcaccatg ttagccagga tggtctcgat |
| 60061 | ctcctgacct tgtgatccgc ccacctcgtc ctcctaaagt gctgggatta caggcgtgag |
| 60121 | ccaccgtgcc cggccgcctc tcggcatacc tcttattcct agcatgtggc tctcacacct |
| 60181 | gaacatggtg cttactccta aagtatgatt ttttggcatg tcacctgaat tcccaagtct |
| 60241 | ctcagtaaag tctcttcata ccagctgaca ggagttccaa aatccccccg tactacatga |
| 60301 | cctccagtat caccaccatt caccttttag caataaatca ggtgatcttt tctagatctt |
| 60361 | ccagaatctt tctctgtgct tgtgcagccc agctcaacaa tacataatgg atctccatgc |
| 60421 | agatttctgc agtgccctct ctgtgcagct tcttcttctc ggttaccctg ctccacaaat |
| 60481 | acagcccctt cataatatca gaactctcat ttacactctt aactcagtga gacttcctat |
| 60541 | tatgctttgt ggcagaaaag tatttcaggc agaaaaccat ggcaatcatg ggatattgtg |
| 60601 | tgacttgctt tatatctttc tcttaaagga cacagctgtt tgctcatgct cttcagtgct |
| 60661 | tgagaacagt tatgtcatat attttgttcc attttgtaga ttttatttgc cagtagagaa |
| 60721 | agtctatata agttgttctg tcatgcccat atctactttt gcattgcttc caaaatgatc |
| 60781 | Wgaatgaaac aaaaatctat gatatcattg ctcactttaa cacttactat tgtcatccat |
| 60841 | tgcctatata atgacattca aactcctaaa tatgagactc aagatccctt aatttataac |
| 60901 | ttttctggaa tcactttcca tactcttttc ctgtagtctc tgcctgttca accctaatgg |
| 60961 | ttttcaatgt aatgtgcatg aggatcatat agtaacatac gtttaaaatg aatatttccc |
| 61021 | tttctacttt actctcattt aaattggtac atctagggta ttctaggaat ctaaattttt |
| 61081 | aacaaacaac ctaggaattt tgatgtaaac aatctaggtc tacatattgt ggattactgt |
| 61141 | tcaagcgtgg caactcaaat tatggtcaca tgatcagtag catccacccc aactaaaaga |
| 61201 | tttcagaaat gctgctctca tttcctttcc agccctattc aaccagtact attcaggcgt |
| 61261 | ttctagagaa atttaaccaa taggattaat ggatctatct atctatctat ctatctaatc |
| 61321 | tagagacata tttattatag gaactggctc acactattat ggagcccaaa agtcccacga |
| 61381 | tcttccgtct ataagctgga gaatcaggaa agctgatagt atgatttggt ccatgtccaa |
| 61441 | agacttgaga accaggggag cctatggtgt aaatcctatt ctgagtctaa aggcccaaga |
| 61501 | acgaggatca gtgatgtctg agggcagaat atggatgtct cagctgaaat aaaaacaaat |
| 61561 | tcaccttccc tctacctttt agttctattc aggccttcca aagattagat gatgcccacc |
| 61621 | catattggtg agggtgatct tctttttcag tctactaatg aaaatgctgg cagggcatag |
| 61681 | tggctcatgc ctgtaatccc atcactttgg gaggctgagg caggtggatc acttgagctc |
| 61741 | cggagtttga aactaacagg gtgaaacctt gtctctacta aaaatacaaa aattagccat |
| 61801 | atgtggtggt gtgcgcctat agtcccagct acttgggagg ctaaggtggg agaatcactt |
| 61861 | gaagtcagga ggtggagatt gcattgagct aagattgtgc cactgtactc cagctgggca |
| 61921 | atagagcaag accctgtctc aaaaacaaac aaacaaacaa acaaaaaact aatgccttct |
| 61981 | ggaaataccc tcacagacac ccagaaataa tgttttacta gctaactagg catcccttaa |
| 62041 | cccagtcaag ttggcaccta aaattaagca tcacaaccag aatgtacatt tttaaaaaaa |
| 62101 | tctcagctga tgtatttgta gattaaagtt tgaagattgc cattttagat aactccattt |
| 62161 | tttaggccaa acagttccac atgtctttat ttttgttttt ataatttctt ctgcttaaat |
| 62221 | attttttaat ttcttttcct gatgcaaaat tattacttat ttaaaaccaa gataaactga |
| 62281 | gttttattga actcgcagac agaaattatt gcactctctt ttgggtttca gttattaagt |
| 62341 | ttacattatc ttttattgta gcacttataa ttatttattt atttgcatat ttcaaatgtt |
| 62401 | ggtctgtgaa ctttggacac tgggactaag tctttttctt tgattcctca gtatatgtga |
| 62461 | cctgtaaagg atccttgaga aatacttgtt tggaaattag tgagtgtcca aacattagaa |
| 62521 | caatacttga catttgttga tattatgtag cttaagaaag actgtcagct tagaagctag |
| 62581 | aatacttctg ttcatatctc agttatgaga cttgctaatt atgcatattt aggcaaattg |
| 62641 | agttttcctt atgctcctca attttctaac ctctaaaatg aagcagatag aattatctat |
| 62701 | tttattgtgt agtttagagt tttaaggaaa caaatcatat gcagtactta gcaaaatacc |
| 62761 | tagcatatag ttaatactta ataaaaatta accattattg ttgttgaact ctaactttga |
| 62821 | caaacattta ttggaaacta acatgttttt cattttgcat agaaaaatta aaagaaatgg |
| 62881 | attggatttt ctggattatt acattctaaa gagatgagaa aaggtaatcc taacaatata |
| 62941 | aaagaaatgg catctatact tagatcaatg gactacaaac atcttttatc acgtcctcta |
| 63001 | taaaattagc actcccaaac acatatatgt gttcataaat tatatacatt aacaaaaaaa |
| 63061 | cccatcagtt aatagcttaa gacaaaatct actctttgga aaacaaccag tgttaaagtt |
| 63121 | tgtagctata catttatatt tcaaatagcc tttttgataa tataaaatat tttgacaagt |
| 63181 | ttattacaaa tatgattaga ttaggaaatt gatttatatg aagctcagat tggtagaaaa |
| 63241 | aagtaccagc tttttctttg ctgcttctta ccttagtaat atatatgttg tgtattttct |
| 63301 | tcacaggact cttttaaaga tacctgccaa tttcgtctat tcagttgaag tgtctattca |
| 63361 | gttaaaattt gatattaaaa tttataatcc cacttacttt ggctcacata agctacagca |
| 63421 | catccccaat aactaaaagc taagaaaaaa aaaagagtga gaggcagctc tcagtccttt |
| 63481 | cccatgtaga attttaagta tatattatac tttgggtact atatgtgaca tatgactagc |
| 63541 | tgatatatga actgaagccg ccagtgtaaa tttacatgtt aaacatgaat gaaacttcat |
| 63601 | ttatctttat atataaatgt atataagcag attttctaat tctttttgta ccctaatgaa |
| 63661 | ttttattaca agattcctgt cacatttata tccctctata gggcatactt ctttaggtgg |
| 63721 | tatctaaatt gtattcctaa aatgaaacac aagactttgg ggaaaaaatt gagacctctg |
| 63781 | atatagttta gaagatctta attgagaaag ctaatgaaca aaatcctatt agaccatcta |
| 63841 | atttagcctt acatgtaact ctcattcaat acacagacaa gccacttagt gaaatttctt |
| 63901 | tagtgagaaa gaagtagcat tttgattcat taacttattt ttaacatttc gtatgataaa |
| 63961 | agcagatatt gaatggtaat ggtcatttat cccatcctat atatttcttg caagatttaa |
| 64021 | ttcctatcat tcagaaacaa ctcattttat atatgtatta caaaagggaa ggacatgaat |
| 64081 | gggggaaaaa tcgaattaaa cctatgttgc ctggcttacc agacctaggt attttttgct |
| 64141 | acgtaactgt gcccatcaga aaactctgac ttttctatca gtggcgattc gtagcctgaa |
| 64201 | tcattgtatt aatatctttt atcctgactt tttttttcct ccttagcaca tatatttgat |
| 64261 | tgcaaagttg aaattttcaa ctatcctgaa actaaagacc caaagtaagt gtttcatagt |
| 64321 | ttctaactgc tgtcgattta gaccaaggtt caacagcctt ggcactactg acaatttgga |
| 64381 | ttggataatt ctttgctgtg ggaggctgtc ctttgtgtta taaaaatgtt tagcagcatc |
| 64441 | cctgtcctct gttctctaga tgagagcagc cacttccctc atgcccagtt gtgagtacca |
| 64501 | ataatatctc cagatatcac ccctgagagg caaaaatcac tcccagttga gaacaactga |
| 64561 | tttagaggac agatttcaat attcattaac ttaaatggat tttttaaatc ttaattacct |
| 64621 | ttatatttga acaaataaag tttcctgatt aaagactgca ttttccctaa atgggaattt |
| 64681 | gacaagatag taataagaaa taatttgaag aaaaaaaaaa ctcaaaaaaa aaagaatccc |
| 64741 | actttttgca tatttaacta aattctccag ttagtgatga cttttaaaca ctttaatagt |
| 64801 | agattttcac ttaagaaact agagaaaatt tttactcata aattacagca atcattaaga |
| 64861 | tagcttgttt atatgcagat ttccctttag taatgaaata tttcataatc tctttctaaa |
| 64921 | tgaacacatt ctcaataaaa agttaatagg atcctagctg aagatggtaa tttgcaattt |
| 64981 | atgcttcaat tettacacca gacagtagta tttcctggaa aaccttccct gatccctccc |
| 65041 | aagtttgggt taggtattgc ttcattgtac tctcttagct cctagtactt actatgctgt |
| 65101 | gtatgctact gtatattatt acttagttaa atctttatat cttcctctat attagcggtc |
| 65161 | acaatctcac atattttaag gatgacataa ataaatgaaa gacagacaat gataaatggg |
| 65221 | aagtaaaact caagactgaa tatttttgag atatgagaga gtggcaaaga ctgtggtaac |
| 65281 | ttagaaaaca tatgccccat gtaaagggaa tggtcactct tcagcttcag ctaatcattg |
| 65341 | tcatgtaaaa atgtagaccc attatttccc aaaatttcag agaatcataa attatgtgaa |
| 65401 | gtttcctaat atttaaatgc tggccattaa tgcagatttt tagtcataaa tactcaacag |
| 65461 | gctaagccat atatatttcc attcctcttg tagcctgttt gtaatctctg atagtaatta |
| 65521 | tgagcatcat caaggcagag accagatcca agttgcttat catcttattc tgagtgctta |
| 65581 | gaacaacgct tgatatatat acttaataaa ttgatgtttt ataagtgaag aatgaagtta |
| 65641 | tagttctctg gtaactgggg ggaagaaaag tgtttcaaag gaggtaggat gaggttgaca |
| 65701 | gtgggcaaaa tggtcctatt tgggccatta gaataggaaa ttaggaaatt gtcagatacc |
| 65761 | tctttaaggg atattttagt aaattttggg gagaagagga tataaaaatg ggttgaaaat |
| 65821 | aaatacagtt agggaaatgg aggcaataaa cattaatttc tcttccaaaa agtataagat |
| 65881 | tgggcaatat ttgaggaatg gccagatcag gaaggtttct gaaacagcat tgtcctatct |
| 65941 | ttttaaactc ttgctcctta aataaacata aaagttcaga gtttatttaa atgttatttt |
| 66001 | taaactttaa aatagatatt atgttatagc taaaaacaaa tttaaaaaat tagaatattc |
| 66061 | attatgcctt ttcaagtagc atgtatttac ttcccccgcc ccactgcaat aggtagagat |
| 66121 | tcttattatc ctcttcctgt taaatcatct ctatattatt aaatgagaga tgatttaagg |
| 66181 | atgttcaaag gccaattgga ggcagcctgc aggaataaat acattatgga aaatgagaaa |
| 66241 | atagaagaag gagggatggc agaggtgagg ggaaaaaata ggtccaaagc aagggtacag |
| 66301 | ataatctaag agtatgttac ttctcttttt ttcttttttt ttttccaaaa tttcaagcaa |
| 66361 | agtcaagtat ttccaccaca ctgaaactct taaaaactga tatcttttgt gaaaataaat |
| 66421 | ccagaattgt ttcaacagga gacatatagg tatgaacctt tgtgagttga gcagttattt |
| 66481 | tcatactgat taatggatac tttcactggg gatgtctatg atgcacgagg cagctctagt |
| 66541 | ccattaaatt ttaatgccta gaactaagtg catttcatac tgaagccctg aatagttcct |
| 66601 | tgtcctaaag gaaaacttaa taatattctt caaagcctcc aaaattactt ttctaaaaaa |
| 66661 | acctttttct ttataaaaac aaataaactt taacaattgc tatgctggtg aagaaaaaat |
| 66721 | atacttagag aaacatttta aaaactttta tttatatgtc cctattttat atccagaaac |
| 66781 | ttagttttct ttccaatcaa agttcagctc cttcttggag tttggtatga cacttcaggg |
| 66841 | aatattagag gtgaataagg tattacagaa aaaaatttag cccaactctg taaccctgca |
| 66901 | gataaagaaa tggaagtatt ttatgactaa acatctcttg ggacagagtg gactaaatca |
| 66961 | taaggtgaat attctcatag aagtaggcca atcttggggg tgatacattt aatatgaata |
| 67021 | taagcttaca cctgtaagta tcttgagttt ataagcattt gcacatacat tatttcattt |
| 67081 | gatctttatt acaatcttgt aaggtaagta aggtaggtat tatatttctt tttataaaaa |
| 67141 | tgaggaatat aaggtcaggg ctgatacatg aaagacaaat tattagcagc tgaagggtaa |
| 67201 | ttggtgggga gggagaaata acatgatgta ttaagaaaag cataatatta aaaatcagaa |
| 67261 | gacctgattt caaatcccag ttcagcctca gtttaaagtt acattaaaga aacacatttt |
| 67321 | atgctatgga atgccaattg ggttctctaa aaagcattct ctgaaaagaa tgtaggagta |
| 67381 | caagaggttt atttaaaggt gacacctgtg aaaggaaaag gaatgaaaca gaattggtta |
| 67441 | tgaggtgcca tcagatccca atacagagct gctaaaatct ccgccagccc attgaggagc |
| 67501 | tccagaggaa aaattgccta tgRaaggagt gccacacggg ctagaaatgt cgaggccctt |
| 67561 | cttttaccac caccttactc agtcattgac tgggagtaag gtccaccctg agaagagact |
| 67621 | gaacacagct ctaatgctgc agtgggtcct gaaataactg agaaagctgc cagtgaacta |
| 67681 | ccatcctgcc acactcttca taaagggagg tctcagtggc atatctattt ctgccacaca |
| 67741 | tgaggaaatg ctcatgattc tataatatat gaaaaaagga aattacctat atgtagaatt |
| 67801 | agatcaatag tgtacataat aacaattatt atgtgtcaag cattgtacta aatgaattac |
| 67861 | agcattatct gatgtaatcc tcacaacaat gctatgagaa tgatacaatt gtatcttcat |
| 67921 | tttacagaca aaaactggac agctcaaaag gttaagaaac atgtcgcaaa tgtaggaagt |
| 67981 | gcccatcaat attgaaacac gagtttgctt tgctgggata gcttgtgctc catctggttg |
| 68041 | cacaaacata tgtacatagg acaaagaatg gaagggaaaa tacacacata aataatagtg |
| 68101 | attatatggt agtactatgg gttattgaat tatttattca ttttctttgt tttttacatt |
| 68161 | ttcttctctg aggtattctg gttgtctgtt gaggcataac aaactatccc aaacttggtc |
| 68221 | ttctaatgca actattttat tattctcatg gattcttagt tcaaaaattc agacaggatg |
| 68281 | ccgtgtggat ggcttgattc tgtcccacaa tatttagagc ctcagctgag aaaatgtgaa |
| 68341 | tgaatgggtt tattcatcag ctggtagctt ggaattctct ggaggcttaa cacactgact |
| 68401 | ggaataagtg taaggctgga cttagctaga acagttgatt ggagagcctt catgtgcgat |
| 68461 | gtaccttctc catgtggcct gcattttctt ataccatggc tgacacaggt tagtcagatt |
| 68521 | tgttacgtta tagatcaggg ctcccagagt gtgtttttca gcaaacagaa aggaagctgt |
| 68581 | gtggtcttct atgtcctagc cttagaaaag aaacactctg ctccacgcta ttgatcacaa |
| 68641 | tccacccagg ttcaagggga aggggcatag atagcaaata tctgtgggtt gagtatagat |
| 68701 | gaatgctcac catgttttaa aaccaccaga aggtatattt tgtccataat cagaagaaag |
| 68761 | tacattttta tacatcagtt tcttttctat actgaaatag agttgaaatt tttttgttta |
| 68821 | ataaataaat tttaaaaatg gatgtaatat ggtagtagag gatttgagct ctgtgatata |
| 68881 | gtggttgctt atcttggctt tatccatagc aataatcaca gagctaacaa taaggcagga |
| 68941 | tattaagtaa gcattgtttt agagttttat ggctagtaca gagttgcagt tactaccatt |
| 69001 | acaaaatatt tttatccatt tacatacatt ttgggagatc tattctacct caaattctat |
| 69061 | ttctcaagga tatagctgga gYgctttttt gtctaagcct cattcttcca cacatggatt |
| 69121 | ttcttctgct taaaaataat aaatatgaca cacacaaata cattacaaag agcttattta |
| 69181 | tctcacactt atagggtgcc tctgtgtgtc acattgaata ataatgcttt aaatttggct |
| 69241 | agagagtgca taaaggaaac ttgtcagttt ccacaagact cttttctaaa caagtcttaa |
| 69301 | gagactttct tttaaaaaca ttgccattag acatttctta tatatggtaa actgcaagca |
| 69361 | ataaatgaga tcaaacttag agtattcaaa atttgcctaa tatttgcatt ttacaggaaa |
| 69421 | attttttcca ggaggtgtgt accatattgc tgactcttaa aattattcca ttaggacaaa |
| 69481 | atattccagt agagtaagag gcttatcagt taaatacagc aaaagtatgt cctctcaagt |
| 69541 | ttttccttga gactcgatca ggaaaaccac tatggaaagt cagaagtgaa gtacagacgt |
| 69601 | tggagtccaa cagacctggg tttgcatcta ccttcaacac ttacaaagta aggttcagtt |
| 69661 | agcactttaa attctttgag cctgagtgac atcggctata aaatagggtt aataattatt |
| 69721 | ttctctcata ggtaaattgt gaagcttaaa tgagaggata tagatagaac atttttcatg |
| 69781 | gaagttggct catagtaagc aatttatatg cagcaattac tgctttatgt ttattatctt |
| 69841 | tagcatagct gcataagata aaagttttat tttggcagta ttcagttaaa aagtatttag |
| 69901 | atcaattaga ggtcaatctt gagaaatcca accaaggtat gaaaggataa attattactc |
| 69961 | ttaacttttg accttaccat ttgcccattc tgaaggaatc ctaatRtact ttaaaaaaat |
| 70021 | tccacaaata ttgactgaat aaggagaaaa aacaaagata cttgaagaaa ggataaatgt |
| 70081 | gtgtttacta aaactgccta atagataaat gtttcacatg ctctgtgatt tacataaaat |
| 70141 | ttattttaat ttagcctagt acagtgcttg tcataaaata gacattcatt aataaatatt |
| 70201 | tgttgaataa aagagtatcc atctttctac tgatttatac aagttattta tgtattaaat |
| 70261 | atgtaaacaa tttgtcagtt ttatatgttg tgaatatttc tccaactcgt cacttgcctt |
| 70321 | attgtgtttt taacacttta atttgctcaa atctatcata ttttctattt aaaatataac |
| 70381 | tttatgctta aaaagacctt cectactact ctgcattgaa atttctgacc atctgcMggg |
| 70441 | aaaatgtttt aaggcgatgt ttttcctatt tatctcaaga ctaatcatgc ctttagccct |
| 70501 | caccaagatt attggtaaag tgagagttaa ctcctgtgaa ctgttttctc tggaggagga |
| 70561 | attctcagtt tctggcagcc tatccatggc taaactagta gacgttttct cgcttccagt |
| 70621 | actggtacag cctttttcca tgttttattc ctttgtttgt tgcagtgcct aactcgtgaa |
| 70681 | gcagagggag tggagttagg aggaagaggt aaagggaaag gagaaagaga agctaagtga |
| 70741 | ttaaatcaaa tcaatatttt atttggaagg tactataggt attatcagaa caattttaag |
| 70801 | gtggttacaa ttactctttt tttatataat caaggaaatt agcaaaaagg agtgwcagaa |
| 70861 | ctgctccagt gcacatagtc attgtgagag ctgaaattct aattagggag gctaattcta |
| 70921 | tgacctgcat cttaaccctg ccttatatgc agctcaaatt taacatgtga aaacgaaaca |
| 70981 | ttttacttct gcctcaaaat ctgcttcttc ctggttcttc cctatctctc tatcattaat |
| 71041 | ttccctacta cttattgcac ttggaaaaaa atccaaatac cccataccat ggcctagaag |
| 71101 | gccctgcaag attatacatc tttgacctca tcttgaaaat ttcttttcct ttgtttaatc |
| 71161 | atttactcaa cattggcctt attgctggtc ttcaaacaca acaagcttat atctgtcttt |
| 71221 | cgacttttgc actagctatg tttcatattt taatacttat ccttgaaatt gtgacttttt |
| 71281 | gccatctacc atttatctca aaagtcacct ctgcagtgaa gctttccctg attaaccagt |
| 71341 | ttaaaatagc catctaatca tttgtctatt tttatctcat atcagttggt actttaagct |
| 71401 | actgtatagc aatttcttat ttgtttactt tttgtctctc cattcacgta aacatagtaa |
| 71461 | aatataagct ctgtgagagc agcgatttca ctatctttac tcactgcagt gtcaccactg |
| 71521 | ctcagaaaag agtttgaaac accagatatg ctcaatcaat atttgtttag catataaagg |
| 71581 | gtatattgaa aagagaaatg cataatcaaa actgggtgta ggaaaatgta tctaattgta |
| 71641 | tgcaggatgg actgcaatgg gaaaaagtat tagttgagga gactagttag gaagctgcgt |
| 71701 | aatacagagg atgatgctag aaataagaag aaatgtgaga agtataagtc agttaaaacc |
| 71761 | agtagaattc tgaaactgag ctgaagggac tctaggtccc ttggaataaa aaagtgccat |
| 71821 | tcatataaat aaggaatgtt agatgacaag tcagcctggg aaaattataa acgataatta |
| 71881 | taagaacaat ttcaaatttt gcatttgaga tgccattgat aggcaaaata Yatgggactc |
| 71941 | agacttagca gagaagtcag gaccaaagat gcatttatca gattgtataR taattaccaa |
| 72001 | agtggcattt aggaatggga aagcagctta gggctacatt tccaaatcat acacttttag |
| 72061 | aagacagtca gaaacaaaga ataggcaaca aaaaggaatc aaggaatgta tagttttagg |
| 72121 | aaagcaaagg agataagaat tttgagaaga aagagatgat ctaatcatag agagaatgac |
| 72181 | tacaaggaca ctggatttga tagtttacct ttagaaattg aagaagtgat ttcgggagac |
| 72241 | tggtgaggtc cttaatgcct caggtggaag aaacagtcat ggtgacttca cctggtaata |
| 72301 | cgatgaaaag agatgtggga aataaatctg ccttagtgag gagtagtgtg agaaaagcaa |
| 72361 | aattcaatgt agaataagta tggtgaactc atttgagatt tatgtggaac ttgggtatac |
| 72421 | ctacctaaag attgctaaag aaatgagact gcaactgtca acagattcaa tatctttttt |
| 72481 | cagaaggctg gaaaatgaag ggaataatag agataaaatc agcttagctt cagtgggaaa |
| 72541 | aaaaaaacaa aacttttttt ttaaaacgga gaaaacataa gtttatagtt caagcatata |
| 72601 | gagccaatga aagaagaaag gttgaagatg caatatttat ttcactttct tgggagagca |
| 72661 | ggtagaaaaa cgggaagatt tggagactat aacattaatt ttactatttg tagctcttta |
| 72721 | ccaaagtttt ttcatttttt agaatttctt gtaggccttt atgcaggtgt gacagcatta |
| 72781 | agaaatcaaa ctttgccttt taatggtaaa attttcttaa caaacagtat ttgatgttat |
| 72841 | atgtatgtaa gtcataaatg cactggccta tcttatgact tgactttatt gctacttggt |
| 72901 | gtgttgatag accaaatatt atagaagttg tggtttaaat tcctactttg aggcttaaac |
| 72961 | atggaaatct ttgtataaca aagattaaat acaaaacaaa attacatatt gaatttcaaa |
| 73021 | ataaaaattc aatgactgtt aattgctatt aatattataa atattatttc atgtatctcc |
| 73081 | aaaggcagaa atacaataca aataatatta taaaaaagta agaattactt taattgttgc |
| 73141 | acaaattttt caaatttgga ggctcaaata atagttctaa catattcact ggtaaaacga |
| 73201 | gtcaagttaa gaaagatatt atgaattaat tgtgcctaga tttaatgata ttatttatta |
| 73261 | gtaagaagaa attactgaac tatcatgaaa cagcctactg ggctgcatgt actttgttca |
| 73321 | gtacttggtt catactctct tcttaaatgg catatttcat cctcatctct aaaagaaaaa |
| 73381 | aaataaacct ttaaactgtt actgtaaatt cctgacctca ctgggagtta attctagttg |
| 73441 | ctggaaaagc atattcattg gagtaaccta cctgtaaaac tgagtttgtg gttgcaagag |
| 73501 | tctcaccata ggtaatgtcc tttctgtctt tcttgttcaa gcggtagagg attgattaga |
| 73561 | actgaRcacc tctgtcattt ggtcctactt tccaccatct gagaactatt ctctggtatc |
| 73621 | taatgtgctc ttatgctaga aatcacactt aagtttcctt tgagcacctg aactggctga |
| 73681 | taggcagaaa atcaatacat cctgttgcac agcttttgaa cctgttatta gcagaaaagt |
| 73741 | aacactaaag atatgtttcc ttacaggata atgcagaatt gatcacttct ctgcttcaca |
| 73801 | gtggagctga tatacagcag gttggatacg gtggcctcac tgccctccat attgctacaa |
| 73861 | tagctggcca cctagaggta agtcatgcct ttggcacctg tgaaaacaaa ggttatttac |
| 73921 | cttttcggtt gttgttctta actgttttga aatactgtct ttgagacttt acaagttggt |
| 73981 | ttatttttta gctactgaac acattggggt aaaatttagc tgtataaatg agctttggat |
| 74041 | agtcaattca gtcaattttc taacagtgta tttaatcatc aRttaacata aaggtagcta |
| 74101 | gatatacaga ggctttatag cagctcagtt tccacaactt gctattttaa ccttggggaa |
| 74161 | gttactgaat ctcatgacct ccctcttttc ttcacttata aaataggaat attagtaagc |
| 74221 | ctattataat gattaagtta gataatttat ataaatcact ttgtagtttg cttaatgcag |
| 74281 | agtaaggaca aaatgaaaaa tcattcactc attcatctac ttatgtatta cttgaatgaa |
| 74341 | tatttattca tcacctatta tgcaccggat actgttttag atgctgggat gacagtagca |
| 74401 | agaaagtcag gcaaaatttt cattttatag agtttagctg taaagaataa aatagacaat |
| 74461 | aaagacaaga aaatattaca taattttaat tagtagtgag taccataaaa ataataaagt |
| 74521 | gaggtaaaat gaaaacaacg agagagaagg gggcactatt taaaataagc tgtacagtaa |
| 74581 | atatctctga aaggaggtca tataggaaaa gagatttaaa tatattgaga atgtcagcca |
| 74641 | tgtaaaagga gagaaaagaa tttcaagctt aaggaatggc acttgcaagg accctgaggc |
| 74701 | ctaaaaaggc tgggtgtgtt tgagggacat gattgagcca gagttgttag agtggagtga |
| 74761 | gcggaggaag aatgatgaag acaaggtctg gtgatgaatg gttccaagcc aaatcacaca |
| 74821 | gtgttttaaa ggtcatagga aggactctgg atcttgtctt aaatttaaag tattaatttt |
| 74881 | atatatccta ttatgggaag gacttccatt tgcatgattt ggctaacttc acaaatcctc |
| 74941 | atcgaatagt ttttattgcc gctgccattt gatagatata aaactaaggc tcttatatag |
| 75001 | taattgcctt gacaaattac aaagctattg aggaaaagag ccatgtttgg aatcctgatc |
| 75061 | attttactca aaatcccatg attgtcacac tctatcaatt gtggaaacac caatagctca |
| 75121 | acaatgtaaa agatgagaag tctgacatcc ttattattaa atagttatgt tccccctttt |
| 75181 | agtcatgttg caattcaatt ataccttctt cactaataca cacacatata cgcacacaaa |
| 75241 | ctgtgaacaa atagagctgc atataaaatt atacttgaag cctgtaaaac agaggtgatc |
| 75301 | catctccctt aacagtgtca cttcaaaggg ctcttacatt caatacaaat taaaatatct |
| 75361 | cgttgagagc aaaatagttt ccttcatgtt gacatttcca ctcctttcca tatcacctta |
| 75421 | tttcctggaa aataaatcct tttagtgaga ttcattgtaa atataggtga gtttgtgtta |
| 75481 | cttatgagga aaggggaaat tccattagat tcacctaaat ataaatgttt tccaaaaaag |
| 75541 | aaagaaagaa actgaggcca gtgataaagt aaatgagtgg taaacattaa gggatctggg |
| 75601 | tgaaataatt tatgagttag acactaactt ctagtaaaac atgatcaacc tcttaatgcc |
| 75661 | aggaaaatta cagactatgt cttcaaaaca aaatcctttt tttcctaatg ttgttttgat |
| 75721 | gatagcatta caatggcttc taaagcaaag tggttaagag cttagtttct tcatctccta |
| 75781 | ttcaaatagc tctgttttct ttctttctaa atcataatat ggaagaaggt attatattga |
| 75841 | ttttaccaga cttagggcaa acttcaccaa agtattccaa atagatctct actcttgcct |
| 75901 | tgcaattctt catgggtttt ctaattttcc tcattagctg atatttgatg acttttatag |
| 75961 | tcaatagcta acactgcaaa aagcaagtat agatacatta gcctttattc tcttcttata |
| 76021 | tgggggacta attaaattat tgatcaaata ataaagaaaa gggtatccaa aactatctaa |
| 76081 | aataattata ttcaaaatga atatttttgt tggagtattt ctttgaaaac tattaccatt |
| 76141 | gacttttgac caatgtggga gttaggagca ctgatcccct gtacagttga aaatctatat |
| 76201 | atacattttg actaccctaa aacttaatta tttatagcct actgctgatt ggaagcctta |
| 76261 | ttgataatat aaacagtcca ttatcatgta tttttaatgt tacatgtatt atatactata |
| 76321 | ttcttaaagt aagctagtga aaagaaaatg ttataaagaa aatcataagg attagaaaat |
| 76381 | atatttacta tttattaagt ggaagtggat aatcataaag gtcttcatca ccattgcctt |
| 76441 | cacattgaat aggcaaagga ggaggaggaa gaggaggggt aggttttgct gtctcagggg |
| 76501 | aggcagagat gaaagaaaat ccatgtatat gtgaacttac atagttcaaa cctgtgttgt |
| 76561 | tcaagggtca actataattg tagtaaatag tcactgtcaa tatatcttta cccatacgtg |
| 76621 | tgaattcatt taacattttt tcagccccta ctttgtctat aaatggttag aatatagaaa |
| 76681 | tataggaatg atacaacaaa ataagacata gatttttccc ctaaatattc tgtaatgcaa |
| 76741 | ctgggcacac aaaataaata tgcagaaaaa atatataagc aattcaaatg taaaacagca |
| 76801 | atatagttat aagtaaggag taggatggat gagagtaggt ttgataattg ggaatacagg |
| 76861 | tgttcctcaa cttatgatgg ggttatatct gagtaaatct atcatgagtg gcaaatatca |
| 76921 | taatggaaag tgcatttaac acacctagcc tactgaacat catagcttag cctagctgac |
| 76981 | attaaatgtg ctcagaactc ttacactatt ctacagctgg gcaaaatcat ctaacagaat |
| 77041 | acctcttttg taagaaaatg ctgaatatct tatgtaattt attgaatact gtactaaaaa |
| 77101 | tgaaaaagca gaatggtcgt atgggtactc aaagtatagt ttccactcat atgtatcact |
| 77161 | ttcacaccat tgtaaagtca aaaaattgta agctgaacca tcgtaagtta gggattatct |
| 77221 | attattagaa acataataat gtagagaaaa tgtcagagaa cacaaaatag aagagttaag |
| 77281 | agttcagtga ggatagagat tgtattagtc cattttcaca ctgcttataa agacataccc |
| 77341 | gagactgggc aatttacaaa agaacgagat ttaactggac ttacagttcc acatggctgg |
| 77401 | agaaggctca cagtcatggc agaaggcaag gagaagcaag tcacgtctta catggatggc |
| 77461 | agcaggcaaa gaaagaataa atgagacgaa aaagcagaaa cccctgatca aaccaccaga |
| 77521 | tttcctgaga cttattcact gccacgagaa cagtatgggg gaaaccaccc ctatgattca |
| 77581 | attatctccc actttgtccc tcccacaaca catggggatt atgggagtac aattcaagat |
| 77641 | gatatttggg tgggaacaca gagccaaagc atatcagaga ttaatgtgga ctcaaagagt |
| 77701 | tagggaagga gttatgagag gggatttaaa attgaactag tctaaagaat gatgagaact |
| 77761 | agacagcaaa aataagaggg atctggccca aaaataggaa ggaaagaatg agataagcac |
| 77821 | agtaagggac agtgagtaaa atgataacat gaaacaaatt gtgaagagtc cctctaggta |
| 77881 | aaattaggtg ttaagaatag acgagactaa agaaacttgg agagtagaag tctaaggcgt |
| 77941 | ttaatttaat gcaagaagca attactgtgg tttacaatgt ataacacaat ttctgtggtt |
| 78001 | tacaatatat aacacacaag agtagatggc atggcagatt aagagattag tagaacacag |
| 78061 | aatttctcac tctttaaaga aaattagaag aaccaatgtg atttttcccc cataggaaac |
| 78121 | agggactaat tgagaaaata tttcagcaag agaataattg gagatttgat aagtaacatg |
| 78181 | tttaaaagga ttactttact ttgactgcat gatttagcat gaattctagt gcgattacat |
| 78241 | gatgattaaa caaagaaaga taaaagcttg ggtaaaggat tcaatttgga ggaaaaatca |
| 78301 | attagctcca cacatgttga ttttctgatg acatccaaat tttcaagtgg aaatgttcat |
| 78361 | taatcattta gaaacataca aacggcattt aatggacaca tttgggttta acgtaaattt |
| 78421 | ttagaaatta tatatagaga ggtaatattt aaaaccataa taacagctta aagttgtata |
| 78481 | ttgggcataa tggatggtca taggaccgag cctaagagaa tctccccgga gacttaagga |
| 78541 | attattaaat aaggaattat tgaaggaaac aaagaattgc tcagtgatat ggaaaggact |
| 78601 | aagattattt taatattgca attaggagag tttcaagagt taaaggatcc caggtatcaa |
| 78661 | aaaagttagt gcagaaaagt agagcacatt gggataaaac acacatcgag tatttcctta |
| 78721 | ctatgctaaa aatttaggtg cttattccac tcctattttt tattatcatg gttagcagaa |
| 78781 | atagcaacgg atcagtgggt gttctgtttc attactagtc actatctttt attgcccacc |
| 78841 | tttctctgtg aaacaatagc tgatgctaag tctgacagga ctctttgtct ccacatatca |
| 78901 | aattcagacc ttccattagt gtctcaagtg tgtttgtcct cccataattg tgaattctat |
| 78961 | taaacaatct tacaataaaa tttacattga ctaggcagct gtcttcttca ataatagaag |
| 79021 | agaacatgtg ctgcagattc ctagacatgc tgactacaat ttgggactca gatttaaatt |
| 79081 | tactgtgctt tgaagagtag gcaaatatta gttgtcccag gtctcaatac ttctctttga |
| 79141 | ctgatacatc catctatcaa tacatttgca tatgcttctt tgtacaaggc ctaagatcct |
| 79201 | gtcagcaatc aataaacatg cattaaatcc tctttttccc tttaggtttg gggaatgtga |
| 79261 | gtcatacgct ttcccaggca tggaaaagga aaaatattag ctaacatttg gtggtacttt |
| 79321 | atcattcaca tacatttgtc atttggttgt tttgtgagat aactactgtc agactcagtt |
| 79381 | tattagtgag gaaactgagg actggagaag ctgaatgaca tctacatggg cattcagatg |
| 79441 | ataaataaat aagctggatg ggactccaac ctggggatct accttcacat ctgtagtctt |
| 79501 | atacataaaa gagcgattRc tccccagaga ctcttctgtt tattgctata acacagctta |
| 79561 | tacccacaag acacaaaaaa agtatttgga aagtgaaaaa agaaacggaa tatgttacta |
| 79621 | tataaagccc aagcattact acatatacga ttaaagcaac attaaatgac tcaaaactaa |
| 79681 | tttcataatg cattcactat atgggcataa cctatggaag tataatgtag gtatatgagc |
| 79741 | acacaacaca cacacacctc aagtgtaaaa cactttatac ttaaataact ccttaaaaag |
| 79801 | ttgttactct ggtgttacgt gctgtagttt ctgcagttgc cataagtcaa atcagttttc |
| 79861 | tgcagccgtt gacaactgtt tgagcagtga acagaaaggc atgcagcaaa catgtaaacc |
| 79921 | aatgtgtaat gtatagaggt tgtggagtgg ttttcttatt acagaaaagt gtactttcat |
| 79981 | atacaccttg tgaaggtgag cgtattattc atagttgaag ttattctatc ctaccatatc |
| 80041 | ccattttcaa attgtaaaca cttattcaat cactgcttca gaaatgcttc ccagttattt |
| 80101 | tggaacttat ttctgatttc aaggtaatgc actcagtatt gtttcagttc attctttact |
| 80161 | aaatcatcca aatgtttcag tagagaattt tgtttaccaa gaagcctcRg accctgtgta |
| 80221 | agttattttt taataatcgc tttagtttcc tctccaaagg cacaaatgac agaatgctta |
| 80281 | gaattataaa atatggtttc tctctctctc ttatttttaa ttttttagag agagggtagt |
| 80341 | catgagtttt ttacctaaac agtagcaagt ctgccccaat cttgtgaata ggcaatagtc |
| 80401 | ccttgtgaac tccatttgga atcagaacaa ataatgaaac aaaataatag ttaatgcttt |
| 80461 | gaatagataa ctttgtactg aacatattaa ttgacttgat cctcagttga gagctctctc |
| 80521 | actcactgtg ctgtactgct tctctcctga tccaaacagt aacctctcag ctcacctgac |
| 80581 | cagaatttaa gctttggaca tgatacacag aagcttagaa tatgtcagga aaacatgcca |
| 80641 | tatctgcaga tctgaacccc ctcaaagact tagcacatta agatattctg gtgccttttt |
| 80701 | gtatggtccc cattcttttg ggctgtataa tttttctcta aaatttctgc attacatttt |
| 80761 | aatagctttg ttgagataaa attcatatac catataattc atccatttaa aatgtacact |
| 80821 | tcaatggttt ttagtatagt tgcagagttg tgtaatcatc accaaaatct aattttagaa |
| 80881 | tatttttgtc acccaaaaag gaaacttaat acccattaag tagtcagtcc cacccagccc |
| 80941 | tcactgccaa gacaaccatg aatctacttt ccatccctac agattggcta ttccagacat |
| 81001 | tttatataat tggaattatg taatgtggtc tttttgtgac tggcttcttt tattttgtgt |
| 81061 | aaagttttca aggttcattc attttcagta aggttgattt ctatcaaaat tatgttataa |
| 81121 | ttacatatga ctttatttag caaaacagaa aaagatacag attcaactga tagtgtaaat |
| 81181 | gcaatttctt tctactacct gtctattttc attataaatc aatgctggaa ggaaatcttc |
| 81241 | aactaaaact tcaagtcccc aaactacact tctagtcaca aaaatgtgcc taaattatca |
| 81301 | gactaggaac acccccatga gagaaagtgg ctaaatgggg tatcttcctg aatgggcagg |
| 81361 | ccacctctta cctgggtatt ggacaaggcc cctactccag acgattaatc ctccttccca |
| 81421 | ccacaggctc actctttgca ttcttaggca gttatgactc tgtaaaaatg caaaagtttc |
| 81481 | taatgcttca gttgcggtta gctttttgta gttaaaattt tagtaatcag ttcataggct |
| 81541 | aaagctttgc taatcaaagt ctgatttatt gattagcaat atagtataac cagtgaactt |
| 81601 | ctttgaaaat agaatctcaa gactacctca gacctgccta taattagcat tttaagattt |
| 81661 | ccagatgatt catatgcata ttcaaattta agaaccctcc cctaaagagt attagaagaa |
| 81721 | attagattta aaagagatat ctgaggaaga taaatagaaa caatgaatga atgaatgaat |
| 81781 | gaatagaaag atgtagcttt catttgtttt gctttacaat ttgcttttgg ttggtactga |
| 81841 | tttaagtgct ttagtaacta ttaaagtggc attcagggct cattctagag cccaggatca |
| 81901 | taatcactca ttgtgcacaa tatatgccta gtacttcaca cattgtgggt gctctaaatg |
| 81961 | tatgttatct ctcttttcct ttctaagata taaaatttca tagtagtagt attaaaataa |
| 82021 | gaagaaagtt ataatgcaat ggaactacaa cacaataatt ataacgtaat aaataaccca |
| 82081 | attgaaaaat gggctgagaa agaggcagag caaaatagct gaaatgaact ccccagcaat |
| 82141 | cgtcccctta acgccccaca ggataaccga gttgaacaac tgcccaggca agaaagcacc |
| 82201 | ttcataataa ccaaaaatca ggtgatgatc acagtgcctg gttttaacat caagaaaaga |
| 82261 | ggcactgaag gctgggtgcg gtggctcacg cctgtaatcc caccactttg ggaggccgag |
| 82321 | atgggctgtt cacgaggtca ggagatcaag accatcctgg ctaacacggt gaaaccccgt |
| 82381 | ctctactaaa aatacaaaaa attagctggg cgtggtggca ggtgcctgta gtcccagcta |
| 82441 | ctcaggaggc tgaggcagga gaatggtgcg aacctgggag gcggagcttg cagtgagcag |
| 82581 | agatcgtgcc actgcactcc agtctaggtg acagagcgaa actccatctc aaaaaaaaaa |
| 82561 | taaaataaaa taaaaaagga aagaagcact gaagagggtg ggaaagacag tcctgcatta |
| 82621 | cctaaactct tccccacctc cccactagtg gcacagcatg geacagagag agaatccatg |
| 82681 | tgtctgggag agagagagag caaagtgatt gtcagacttt gcattggaac tcaatgatgg |
| 82741 | cctatcatag tgaaaaacaa tacagggcag aattctgcca gcccccatgg agggagcatt |
| 82801 | tagaccagcc tcagccagaa ggtaaccctc cactgcagca gtaggaacct gacttccagc |
| 82861 | tagctctacc attggttgac tacagtgctg gggtcctgaa aaaatttgaa agacaggcct |
| 82921 | cagggactac agtacttggg caagtactga tgctcttctg ggcttgcaac cagtggactt |
| 82981 | ggggtgcacg tgacccagtg agacaccagc ggtggtgacc aagggagtac ttgcatcacc |
| 83041 | catctcccaa ctctgtgcag tgcaacttgg ggagagactt cttttgcttg ggaacgggag |
| 83101 | aaggaagagt atagaggact ttgtcttgca acttgggtaa ccagctcagc tacagtaaac |
| 83161 | taaagcaact agcagactcc ggaagccctc tcctgattcc aggccctggc tactgaacag |
| 83221 | catttctaga cccaccctgg gacagaaggg aacccagtgc gctgaaggga aggacccagg |
| 83281 | cctggaagga ttcaccactt gctgattaaa gagccctcag gccttgaata aacatgagtg |
| 83341 | gtagccaaga aatagtcacc acaggctttg ggcaagaccc agtactatgc tgacttcagg |
| 83401 | tgtgacccag cgcagtctca gctataggaa agtaatttat cctcccttgg cctcaatttt |
| 83461 | ctagttttag aaagtggatt atactatcac ccacaataat agatgtgtgt gtgtgtgtgt |
| 83521 | gtgtgtgtgt aatgtcaata tatgtcaaat gcttagacac tacctagcac ataaacttta |
| 83581 | ggaaagttag ctctcatttt tatttgttct gttttgtttc tgattttact gttatatagt |
| 83641 | tgtaaagaaa aaatgtggta tcctaggtag ggtaaaacat taatgtacaa tttaacttag |
| 83701 | ggaaatttaa tgtccccatt atattgagtc ttctgtttca gagcaaattc tttccattta |
| 83761 | ttcaagtttc tttttcgatc tttcagcaga atattcttct cataaatttg ttaaaattta |
| 83821 | ttcccaggta ttttatcttt tcattgcaat gtgaatgaca ttttctagtt tgttatagtg |
| 83881 | tgtgtatcct ataaacacac tatataaaac atattaaaaa tcaagaaaca ttggttcttg |
| 83941 | atatttaagt ttaaatcatt aaacttactg atttgtttta tcatatggtt tagatgagtc |
| 84001 | ttttggaact tcctggttta caattaaatt ccaatatatg gtgataattt tgcttccctt |
| 84061 | ctgataatta aacatcttgt ttctcttttt cctaattgca gtggttgata ttttcagaac |
| 84121 | aattatcatt tttcaattga attatcattg tactcctgga atgaaacaca attggtcatg |
| 84181 | gtacattatt cccttagctt gctgctaaac ttgttttgct aatattcata agtgaggttg |
| 84241 | gcctatggtt ttcttttttg tattatggca ttagtttcca aggctgccaa taacaaatat |
| 84301 | cacagactga gtgaaatgta tctcactgta cattacagta gagttagatt agaacacaga |
| 84361 | gaccaagatg ccagacatgt gcacacacag agatatgacc atgtcatatc tgctactgct |
| 84421 | accatggccc atctgctact tatcacttgt tcatgtacaa ctatacattt ctgtacaaca |
| 84481 | gaaatatatt ttctcacagt tcagggcaat agaagttcat tatcaaggtg ttggcgaatt |
| 84541 | tgatttcctc tgaggcctct ctccttggtt tgcagatgca ttcgctgtat gttaacatgc |
| 84601 | tcatatctct gtgtgtgcac atgcctggca gcttggtctc tgtgttctaa tctactcttt |
| 84661 | ttatgaggtc acttgtcata ttaattaatg cctaccctaa tgaccttatt ttaacttaat |
| 84721 | tacttcttca aaggctctat ttgtaaatag tcacatcctg aggtattatg gggttagggc |
| 84781 | ttcaacacgt gaatttcagg ggacacaatt ccccatagca attatccttg tcagattttg |
| 84841 | aaataaatgt catgttgaat ctgtaaaaat aatttggaag cttttctctg tctctttctg |
| 84901 | tctctctctt tctctgaatc tgttatgtag cctgatgttg tttttatttc acacaatgca |
| 84961 | tcaatattac cttgtctctg ataagtctcc caaaatttca caggcagaga aacaagtcct |
| 85021 | gtttctgtag gagaccacca atatgcacat ctcagactac cataatttgg atatggaaaa |
| 85081 | catcgcaagg gccaaatatg ccagccgatg cacatcatta taaattattg tttcaatatc |
| 85141 | aatctaggga atgtcactaa tgattgatag gtattataat caccattcat tgtcattaac |
| 85201 | tttataggtt tccatacttg gtttttgtag tcatgtgcag ttccacaatg ccaaaacttg |
| 85261 | atataccttc attttaaaca gttaagtttc taaaagtaaa cctttctttt acacatagtc |
| 85321 | ttaaatatct aagactcaat ttaagaacac tgcatattct gtggcattcg aaaatggttt |
| 85381 | cctcgggagt tgaaaccata aactctatac cccattactg agtgtgccct atgttacaga |
| 85441 | atactcctta ttaaaggtga aaaagcaatc tactactctg caggcttttg gtctggagat |
| 85501 | aaacacaaga tcatagatca tgtattatta tcagttattt taaggtctca gagcaattct |
| 85561 | ttaaacatgt atcaattgta aggagtaata taaaaggacg ctagcttgtc aatttctttt |
| 85621 | tgtcactcag gtctttggca tagactctgt tagctttatt aaacattaag tattgctttc |
| 85681 | aggKtgaggt agaagagcat aagcaagaaa agctttatag gctaataaaa atgtaaatga |
| 85741 | gatgttcatc agcttgctgc cctggaaaat gatggaagaa acattacctg aaaaattaga |
| 85801 | ttcaacRatt attgctaacc aaatcaacct gatatgagaa atccctttgt aaccatttgt |
| 85861 | gttaatttgc atccttatgt caatctttct atgcactgaa tttgcttcca acatcctagg |
| 85921 | tcctctRtac atatatatgt agataatgta tatacacatt tttatatata catagaaata |
| 85981 | tatacacata tttgtataat tcatgttatt aattaacatg tattagctaa acagaaatat |
| 86041 | ttctgttaca tatttgcagc agttggtaag aaacgcctat atattttaat tcgtctgcac |
| 86101 | ttagccttct ttttgcacac catactcctt acaaggaatt tgaagactga gttgtaaatc |
| 86161 | agaatttcta tagcttgtta aagtcacatc ctcagagttt ggaaacctct gagctcacta |
| 86221 | tgtctcagga agacgggaaa ttcttcttct Wcttcttctt ccccttaatt aggaatacat |
| 86281 | tgtaataata atattttcta tgtgaccttt gaatacagta gtaaaataac aagtgggggc |
| 86341 | tacttacttt tctcagggat gattcatcct gggatggtgt ttagacagtg gcctttgagt |
| 86401 | attggatatt ctataaacat cgtgtgagtt gttcttcttg atgactccct ttagtggatc |
| 86461 | tccgaagacc ctttagaagc ttgctgaggg tgggatggag gaagaggcta aagcccccat |
| 86521 | aactgactct cagcaactgt cctctctcac agaatctctc ttttgcggtg acccttaaag |
| 86581 | gcacttggcc catctgctac ttatcacttg ttcatgtaca actatacatt aggccaatat |
| 86641 | attaggttgg tacaaaaata aatgtggttt ttgccattat ttttaatcta aaaaacctgc |
| 86701 | aattactttt gcacaaacct aatagttatt gtaaaggatg gaacttttaa gcaaggcaca |
| 86761 | caaaaccttc taaagtcact ctgagaagca attcagagta gccccttgct gcatctttca |
| 86821 | ttttgcagcc attttactct tttctctgcc ctaaagcaga tctcaggtta ctaactaacc |
| 86881 | catttatgcc agaggttgca catttttttg tgtgaaaaat cagaccttgg cgatgacctt |
| 86941 | gagaagtagg atataaataa ctcccacaag cttagcgttc caataatgga acactaggca |
| 87001 | taaatgggct aactccttca gctctgggct gagtttctca caattactgc agatctccac |
| 87061 | atctgggcag gtcctgacaa tagttaaaga ttttttttct cataatttgt cctgaaagga |
| 87121 | catatattga gctattcagg agacttgaga aaagttccaa gttaaatagg ataaagggca |
| 87181 | atggatacac attttttgtt cagaaatata aagattattc ttagttagag gtgaatatgt |
| 87241 | tccttcactg ttctctgaat tcattctgtc attctgctcc tggacttcct acaatagtac |
| 87301 | cttgacttat aaggcccgtg tccagatctt ttacagtcca cttcattcaa aatgctcctg |
| 87361 | aaaaattcaa gggactcact aacacatggc tttggtaaag gcattctcct gaacaaaaac |
| 87421 | ctttctgacc tcaaaatMca cctagcaagg gatttagtgg tgataatcta gctctcctac |
| 87481 | cttttcagcc ctggatcttt atgctcttta tcttgcaaaa catgctctaa attaaattaa |
| 87541 | ctgctaccca tttgtatgcc ccgtgtttct acaacactat agtctcttta tggttcacct |
| 87601 | tttctaccca tccccaaagc ccagatgaaa tgccactttc atgaaactgt tggatccatc |
| 87661 | tatcctgaag tttttctatg ctctgaacaa ccacaatgtt tttttgtacc tttctaataa |
| 87721 | catgtaacac cacctgtttc atgagcatag ttttatttga ttattttatt atactctttt |
| 87781 | ttgagcacag gaactgcaca ctagaaatca tcatatttct cacaggttcc aatattgtga |
| 87841 | tttttgcata cagtagattt ttaataagtt tctgtcaaat gactaaacta ataattattc |
| 87901 | aagtgtttta ggaattaaac agaatgagga aaattatttc tactctccat aaaagcagaa |
| 87961 | gagattataa tgatacgttt catgaagaaa gtagcttaca aaattacaag caattttcat |
| 88021 | gaataacttc aaacaaatta gtcttaatac ctataaaaag tatcacagcc ttaaaaaaat |
| 88081 | gatgacaatt aagtgatgtc agcagtatgg cagaataggt gtttccattg cttgtcatct |
| 88141 | cacagaaata tcaacttgaa gaactatcca cacataaaaa taccttcaca agagctaaga |
| 88201 | aattcagatg agagattatg gcatctgaat ggagcagaga aatcagcaaa gacacattga |
| 88261 | agaggacaag aatagtttta cattatgcat gtcacccttt cctaaagcct atgcagttca |
| 88321 | gtacaaatag agatagcctc catgtaaagg aaaagaataa agtgagtccc taacttcatt |
| 88381 | gcaggcccca gcaccatgcc taccccagtg gattccagaa ccaggccagt cctcatgtcc |
| 88441 | ccagacctct ctctggcccc catggaccca gcatccaggc ccatgtcgct gccaaactag |
| 88501 | cccctgtagc ctgtggcctc aggcctcagg ctcattctaa tatcagacca gccctcacag |
| 88561 | ctccaggcct gctctgggcc accccagtgc caggaaggcc cccataagtc aggctgcagg |
| 88621 | ccagccctag tagtcccagg ctactaggga ctagagttca tgtaagccct cctacatgaa |
| 88681 | ctcaggtacg aggctcattc atatggaccc aatgtccagg ccctccccag tagacccagg |
| 88741 | ctccaggctt gcccctacaa aaaaagacat cagattgacc cctgtggact taggcactag |
| 88801 | gcccatgaac ccactgacct aggcagcagg ccagcctgcc tgaggactcc cgtggcaagc |
| 88861 | ccactgcaaa ccccaatgga taacctggtc agaatctctg gacaagctgt ctggtgaagg |
| 88921 | gctttccttg ccagtctgta aagactggaa gaagtgtcta cttcaaatgc acagatacca |
| 88981 | atacaaggcc acaaggatca caaataatta gggaaacatg atgctaccca ggaaacaaag |
| 89041 | caccaggaac tgaccctaag gaaatgaaga tttctgaact gcctggcaaa taattaaaaa |
| 89101 | taatcacctt aaagaggctc aatgtgctcc aagagaacac agatatcaga agttcaacaa |
| 89161 | agaggtagaa accataaaaa gaaatcaatc agaaactcag aagctaaaga gcacaatgat |
| 89221 | tgaactaaaa aatttcacag agaacttcaa cacaaacttg atcaagcaga agagagaatc |
| 89281 | agcaagaaaa aaaagaaact aaggaaaaaa acaggtcatt tgaaattact cagataaaca |
| 89341 | aaaaggagaa aaagaatgac aaacgtggaa aagcctacag cacttttggt acatcaacaa |
| 89401 | gtaaaccagt gtatgcatta ttgaggtcca agaagaaaca gagaaagaga aaggggaaga |
| 89461 | aagcttattg aaagatatca tgacagaaaa cttttcaagt ttggaaaggg aaatgaatat |
| 89521 | ccttatccat gaagcccaaa gaaccccaaa gatacagaga tattcactga gacacatcat |
| 89581 | aattaaattc tcagaaatca aagacagaga aagaattttg aaagcaccat gagaaaagtg |
| 89641 | actcatcaca tatataaggg tataagaata tttattcaac aaaggtggca ggaacacaca |
| 89701 | ataggaacag gacattctct tcaataaatg atactgagac aaatcaactc aaaattgact |
| 89761 | aaagacttaa atgttaaggg ctgaaacttt aaaactacta gaagaaaaca tagagaaaag |
| 89821 | cctttttgaa attggtttgg gcaatgattt cttggctatg acctcaaaaa cacaagaaac |
| 89881 | aaagcaaaaa gagattaaca ggattgcatc aaactaaaaa gcattggcat agcaaaagaa |
| 89941 | acaatcaaca gaataaagag acaacttatg aaatgggagg aaatatttgc aaaccataca |
| 90001 | tctgataagg ggttaatact cgaaatatat aaggaattca actcagcaac tcagtaaaag |
| 90061 | gacaacaaat attccaatct aaaaatgtgc aaaaaacctg aaaagacatt tctcaaaaat |
| 90121 | aggaaataca aatcgccaag aagtatatga aaaaaatgct taatatcgtt aattatcagg |
| 90181 | gaaatgcaaa ttaaaactac aatgagatat taccccatac cattataaaa tggctactat |
| 90241 | caaaaaggca aaaaatagca agtattgaca agaatatgca gaaaagtgaa acggtacagc |
| 90301 | agttatggaa aagagtataa agattcctca aaaaattcaa aatatgacta ccatgtgatc |
| 90361 | caacaacctc atttctgggt atatattcaa aagaattaaa atcagcatat tgaagatata |
| 90421 | tctgcattcc cattttttaa aaatggtgtc tcattctatt gctctctcat tcgtattgca |
| 90481 | acattattca caatagccaa gatgtgaaat caaccaatca acctaagtgt ccatctattt |
| 90541 | atgaatggat aaagaaaatg tgggtataga atagtataca tatcatatat gtgaatatta |
| 90601 | tttatcttaa aatgaagaaa attctctcat ttgcagcaac atgaatgaac tgggaggaca |
| 90661 | ttatgccaag tgaaataagc taggcacaga aagacaaatg cttcatgatc tcacttacat |
| 90721 | gtaaaatctt aaagccttaa aattacagaa gcagagagta gaatggtggt tgccgggggc |
| 90781 | tggatgtgaa gaaacaggga tgcgttggtc aaaggatacg aagttttagt tatgaagaat |
| 90841 | gaataaattc tggagatcta atgtacaatg tagtgtttat aattattaat attatgttgt |
| 90901 | atactttaaa tttgctaaga gagtaaatct taaatgttct taccacaaaa atttaactat |
| 90961 | gtgaggtgat ggatatgatc attaggttgg ttgtggtaat cacttcacaa tgtatatata |
| 91021 | aatcagtcaa agcatcatgt tgcacaaaat aaatacatac tttttatttg tcagttatac |
| 91081 | ctcaataaag gtggaaaaat aaatttttaa acactgaata taggtaaagc taaaagttat |
| 91141 | atgtggggat atggggaaag agtgtatgtt gtggcttgga gcaacaagta aaaggattac |
| 91201 | atcttcatct tccacatagt gtggttaaac agaaaaaaaa taaatattaa aaaaatcaat |
| 91261 | tatttttaca ttgtgtatat aaatatccaa agaagcagct aaattagtca aagtgcttat |
| 91321 | ctttgggaaa taataatgga aggagaagta aaccagagaa ctttgttttt caaagttgac |
| 91381 | cttgtagaac catttttgac ttaaaaaatg gatatatgct gttttgattt ttaaaaatta |
| 91441 | attaaaaaat gatgatcaca ttttgtcttt aaaaattttt gtttaaaacg tatccatttc |
| 91501 | ttctccccat ttaatatatc ttttgaccat ttaaagtata aaaaaagaat ttggacttaa |
| 91561 | tctaatgaag aagtttgacc ctaacaaaag agagtaatgg ggtttaaatt tatgccactc |
| 91621 | tacaaaaaag aagtcgaact cacccatagg tagtgtcatt agtcttttag gtctgcatgc |
| 91681 | ctctctctat gacttttccc aatctttgtt ttaatcaata atcttgtctc cagtctaaca |
| 91741 | tgggaattgt gctctctggc ataaaatggg ctactgaatt acagtcctgt gctatatcac |
| 91801 | acagccttgc cattgatagc atggaaatac ttcgaaatac tgctgtcaaa atttgcaaac |
| 91861 | tgtcaagata aataagttca ttcagattat ttatatctgc tgtttctccc cctaacctca |
| 91921 | tacttagaaa attatatgtg tctagtcctt ctccaacttg attttatgtc ttttagccta |
| 91981 | atacattggc aaaccaaatg catgactctg acaccgataa tactaagaag ggctgaaatg |
| 92041 | aattgttaga atgagagaca gtcactttgt ttagaaggga gtgctccatg agcagtgtga |
| 92101 | taacatagct ccttgaaaat ctagtcaatc accattttat agtaaaagcc tatgaatttt |
| 92161 | tgtagcttgg tgtcttattg tctctattca atagttgctt ctaacttttc tgggcatccc |
| 92221 | cagaaaagtg gaaacctcct gtgaatgagg caaatttagt aacctccaat taatcaagca |
| 92281 | cactaagcac ataccacatc tgcaggaata aactaggtat gggattaaag agatgtatag |
| 92341 | aattaattct ttgcttaatg tattttgcca ttctatattt cttttttttt tttcaagaga |
| 92401 | gcaagtctaa gtcctccctg gagaattgtt gaaataatgc ctaatttagg taactaagca |
| 92461 | ttttaagcct ttctacactt ttataatatt tgtaattgaa aaaaacccag caccaatata |
| 92521 | taaataatta atttaccttt gaaatactgc tgtcagaatt atgatgcaaa ctctgcatca |
| 92581 | catttttgca taccttttaa cccaggaaat tcacttctag aaatttattt tacgaaaata |
| 92641 | ataagagatt gaattgcatg ggtgttcatg caaaataaaa aagtaaaatg ctctaaatgc |
| 92701 | ccagcattaa atattgatta aatcaaaaca gtgcatggat atggtgggac actatgctat |
| 92761 | cattaaacat gacattgtac aagattatac attgacatga aaagacacat ataaagactt |
| 92821 | tgtgaaacat gtataatgtt atttaagctt tgttgtgaca ctcaagtttt ttctttcttt |
| 92881 | ttagtttttc ttattactag aatgaatgca ttttcttaaa tattcacctt tacagaagtg |
| 92941 | actacacaat taatggctac acacctctcc ccacttattt ctgcccaaag acaactactg |
| 93001 | ttgacatttt ggtggatatt ctcctaggtt gtttcatgta tgtctttttc aaatgtagga |
| 93061 | tcacactaaa tgcttctgaa gcatactttt cttatttaaa aaatacagca gacagcttct |
| 93121 | aagtgtgcac catgttcttt tgtaaacata tgtattttaa atatatgcac acacataaag |
| 93181 | agagagaaag aggctgttta aggagtaatt gttaagtgga tatgcatgca acgaccacca |
| 93241 | gtttaagaag taatttctag ttccccggag aatcttcatg agaacttctg atttcatatc |
| 93301 | tgtctccttc ttctagagta atccctctct ctctatggta actattcctt tgctttccta |
| 93361 | gatattaagt atatgtaaat gtatgtgtaa ctctgtgaat atgtatctct gtataaaatc |
| 93421 | attaattaaa tatttttaat tttttctgtt cttgaacttt atgtaaatga aatcaaaggc |
| 93481 | atatttttgt gactttatta tttattgtga cttctttgtg agtgatcact tatgacattt |
| 93541 | taacaatttt tttatgcatg tggctgttac atagccacat aatatatgtt tacataggtc |
| 93601 | ttatgtacaa gggaaggttc taagcacatt atacagatta tcttgtttta tgtcgggaat |
| 93661 | aggtatcatt attcgtttca ttttacagat tgggaaactg agttccagaa aggttaaata |
| 93721 | acttaatgta ggtcacacag ctagtaagtg gtgtagatta tattctatcc taagttgact |
| 93781 | gacttcatag tctgtgatct taaccatagt tccaacctgc tttctgttac agatgagtat |
| 93841 | gctacagatg ccttctgctt atttagggtt aatgctttca tatattaaga atcccccaag |
| 93901 | actcaatttc ataaagatat tttaaattat tatcttaagg tttgatggtt tgccttcagt |
| 93961 | atttaagtgc tttacctacc taatattgat tccagtctat ggtacaaagc atgggtttaa |
| 94021 | cttaattttt tgacatgttc aattgtctca acacctttta ttaaaaagct tatattttcc |
| 94081 | ttattaataa tatacaatac taattctgtc atatatcaag tgtccattta attgcaaatc |
| 94141 | tgtttctaga tctctgttct gttcatttat ctacttatct agtcatgcaa catcctatat |
| 94201 | aaagttgttc agaatagtct cttaataatt ttaattttct accaatagtg gcatattctt |
| 94261 | ttttattcct gatactgatt attttggtct ctctttttgt cggtcttatc agaaatcaga |
| 94321 | agtttagcaa ttttgtgtct ttgcaaagaa ccaacttctg gctttgttga atttctctag |
| 94381 | tattatatgt ttgtcttttt tttaatcaat ttctgctttt atctttctta acttcatata |
| 94441 | atttcactac tgtttttcta atttcttaag atggatgcat agcttagctt attaatattt |
| 94501 | agcctttttt ttctaacagc attttaggct acacatttgt aagttctgtt tttggcacat |
| 94561 | ttaagttgta atatattttg ttactgttca gtttaaaaca tttttccttc tactagaaat |
| 94621 | tctttttgga cccataaatt atttagaagt aggtttccaa ctttacaaac ttagtgtttt |
| 94681 | taaattacct tgatattgat ttttaagtta gttgtactgt aagcaaagaa cttcgtcatt |
| 94741 | agttttccag tatttgaaat gcattgagtg ctgctgtaag ggccaatatt tagtcaattt |
| 94801 | tgtgaatgtt ccctttattc ttgaagtgaa tatgcattct gcagttgttt gctacacagc |
| 94861 | ttataggtac aaatttgatc aaggtcttaa ttgtgtcctt catgtctttc atatctttat |
| 94921 | tgtttcttgt tggctttatc aattttgtgt taaaaatttt tcaccatgat tgtagatctt |
| 94981 | gctgtttatt gttgtagttc tataaggttt tgcctaatat attttgatgY tatgtgttat |
| 95041 | tgatataaaa tttaacatgg tttatagttt cccagggtga attaatgaat acattacaat |
| 95101 | agattgaact tctctatctc tagcaatggt ttactttacc catgaagtcc aatttttccc |
| 95161 | ttatcaattt aataatatgt gactttattg cttagaattt attggacata tctttttcta |
| 95221 | tactgttatt ttaagacttc ataactgttt tactttaaag gcattttgta aatggcatat |
| 95281 | aattgaattt tattctaccc tagacagata ttcttttgcc tgaaagatta attcactttt |
| 95341 | attagatatt attacttaca tatttggagt tgaatctacc aacgtatttt gtatttccat |
| 95401 | ttgctccatc ttgtcttacg tcaaatatta agtgcctgta tatgtgatga tctgtttcta |
| 95461 | gactctatat taacctactg ttctatttgt ttgttgtatg tgagtaccac tgtgttttcc |
| 95521 | ttgctgaagc tttacaatga gttttgacat ccaagcatat gaatgtaata atcttcttct |
| 95581 | ttaagatttt ctttgttact ttttcgcttt ttacatttta ttatatatta aaatcagatt |
| 95641 | ggtaattctg tgatttttga tagaactatt ttttaaattt aaattaaaat tttcatttgt |
| 95701 | ttcttgttgc taatgaataa aaatataatt tttgtacact ggccttgttt ctagtgattt |
| 95761 | tgtttaattc acattttaat gatttatatt taccatggga atgattcgat tgcaaataga |
| 95821 | ttcttctgtc cttagttttc tatttctttt tctttcctta tttcattggc actctagaac |
| 95881 | aatgtcaaac agaaatggtg acaaagaata gacctgtttt attcctgatt ttagggataa |
| 95941 | aattatattt cagcattagc tatagagttt ctataatttt gtgattattt tctaagtatg |
| 96001 | tatagacatt tttatcacat taagaatttt ttccttctat tgtttgctaa gaatttagtc |
| 96061 | cacaattact attaacatta aattgttatc aaatattttc tcatctattg agatcattat |
| 96121 | gtaattttct tctattattt cattaatatg ctaatttaca ttggttttga atgccaaatt |
| 96181 | aattttggat tcctaacata agttgcactt tttctgacat gtcattttta tactcgttga |
| 96241 | attcagattg ttgatgtttt tattttttaa tttacaaata aaaattatat atatttatgg |
| 96301 | tgtacaacat gatgttttga tagattcttg atattttctt tagaattttt gcaactgtat |
| 96361 | ttgaaagaca ttgtcctata atttattttc tcataatgtt agaaaaccct ggcctcatag |
| 96421 | aacaaattag aaagttctat tctttggaaa agtttgtgaa gatttatgtt atttcctcca |
| 96481 | taaatattta ataacattat ctagtaaatt tatetaggat atgggttttc tttctggaaa |
| 96541 | tgtgttcaaa ttatgaattt gatattttca gaagatatgg atagtggttt gtgggaatct |
| 96601 | gttgatactg acactggctc agggtgcatt ttgcacacct ctttctaact ctctcctcag |
| 96661 | tgatgtcatg caagtagctt aaaattagcc attgtggaaa tatttacaac acaaaaatca |
| 96721 | aaaaaagtgt ggcttatttt ttgagagctg aaggaaaact taaatgtttt cacttcttcc |
| 96781 | tatgttcatt ttttaaagtt gtgatttcca aggcatttga ttatttcacc caaatctcca |
| 96841 | aatttattgg cataaatttg atcataagtg cttcattata tttaatgatc tgcaataatg |
| 96901 | atctttttca ttactaatat tatgtatttg ttccttctca atatttttcc tcaggtatct |
| 96961 | ggtcagaaat ttttcaattt gtaattattg tttgtttaaa aaccttttgt gcttttgtta |
| 97021 | tcttctctat tttttttact caatttcatt gttttcttct ttttaactgt ttcttttagt |
| 97081 | tttctgggga aaatataact tggaattttt tctaacttct taagatgaat tattagatta |
| 97141 | tttattttca tccttttttt attgtttaat atttRcattt aagactatag tttttcattt |
| 97201 | aaatacaggg ttagtggtat acctgaattt ttatatattt acacactcat tcacttcaaa |
| 97261 | tattttcaga tttccattat tatttgtctc atgggtcatt tattttttca ttgctaaatt |
| 97321 | ttcaaacatt ttgacagtta ctaattattt ttttactttg atttgcagta taattgcatt |
| 97381 | atgctcagaa attatagttt |

Following is a PCLO genomic nucleotide sequence (SEQ ID NO: 6).

| >7:82022801-82112500 | |
|---|---|
| 1 | agttgcctcc ctttgccccc ccacccccca acaggccctg atgtgtgttg ttcccctctc |
| 61 | tgtgtccatg tgttctcaat gagtaactgc catttctgct aatgcatgta attacatttg |
| 121 | tgttcttatg atatatattg gttttcatcc agggttcctg gtttataact cccacagccc |
| 181 | ttattacagt cttttgttac aatgttgggt gtgtgaggcy tcaggggcag gcctcaggaa |
| 241 | atagaatctc tctcctgccc tcctttcagc tgccccaagg cagaactcta atcttctccc |
| 301 | acctttctga tgtgagtctt aagaccctcc ccagggaagg tcccaccctg tactctgtgg |
| 361 | gaagaaatgt tgacatcatg aagcttccat aaaaacccaa gaggattggg tttgaggagc |
| 421 | ttctggagag ctgaacacat ggaggttcct ggagggtggc atgcccaggg tgggcatgga |
| 481 | agcttctcgc cccttccccc ataccttgcc ctgcacatct cttcatctgt gtccttgata |
| 541 | gtaaacctgt aaacataagg tttccctgag ttctgtgaat tgtttcagca aattaatcat |
| 601 | acccacagag gagatcacag gaaccccaac ttgaagccag tcggtcagaa ctttctaagg |
| 661 | cccagacttg tgacaatact tgtgtgtatt gggaggcagt cttggggact gagcccttga |
| 721 | cctgtgggtt ctgacactag ctccaagtag atagtgctgg aattaaattg gaggacaccc |
| 781 | agctggtatc tgtaatttgg tgtgtgggga aaacagcccc ctaacctctg acgtttggtc |
| 841 | acagaaatct ctgttgcttg ttgtggtgtg agaattgtgg aaaaacatga tttgaggaga |
| 901 | ggtttcttga aataacattc ccttcctttt ccagaaaaaa atcttctata cataaatgct |
| 961 | cagttacagc atgcaaacta ctaataattt atttcattat tttcatttat tttgcttaaa |
| 1021 | tcacagggac tggctatgtc tgttttagag gactgatttt acagtgcatc caatattgta |
| 1081 | aatctactgt ggcaacatat gcttagccag aagaagtcga aagagaagag agtagaatgt |
| 1141 | tctattctgg aaatagtttt catgtcacat ataaaaatga tatactaaat gaattatgta |
| 1201 | taaattctaa atgagctttg aaactttttt acgttaatat aaaatagtat agaatatcaa |
| 1261 | aatccatttc agaaatttac tacggttcac tgttatgttt ttcatttcaa gcaaaactct |
| 1321 | tctaaagttt ccataatata aatatatttg aaagtgtctt atatRttagt atatacagag |
| 1381 | atgccttaac acaatctctt attttatctg tctagtatct tttctcctct ttcaccacta |
| 1441 | tctcaagaac atctactccc aaatgcatat ggttctctta agctggaaac caccatactc |
| 1501 | aattttcttt gtcacgatgt ttgttccagg taagtgcatc tgacccacat aggaaaaacc |
| 1561 | agagtgcttc actgaggatg ttagacatat tttctccttt gggtcaagag tgtaaaacta |
| 1621 | ggcctcaaag gagccagcaa cttcatccct tgccttatag aacaacctga gaaaaggcca |
| 1681 | actttcagag aaaagctgag aaaagatgtg agaactaaac aggcttgagg ctggtctttc |
| 1741 | ctgagactat gatatctaca tgtgaatttc ttagtatggc caccagtaaa cattctttgg |
| 1801 | ttttgagttt gtttgtgtag ttttccatct cttaccagtg aaaatactgc cttatcttag |
| 1861 | agttaaagaa atatcacatt taaaaattgc accctagagg gccgggtgtg gtggcttacg |
| 1921 | cctgtaatcc cagccctttg ggaggccgag gcgagcggat cacgacgtca ggagatcgag |
| 1981 | accatcccgg ctaacacggt gaagccccgt ctccactaaa aatacattaa aaaagaaaat |
| 2041 | tagccgggca tggtggcggg ctcctgtagt gccagctact caggaggctg aggcaggaga |
| 2101 | atggcgtgaa cccgggaggc ggagcttgca gtgagctgag atcgcaccac tgaactccag |
| 2161 | cctgggcgat agagcgagac tcggtctcaa aaccaaccaa ccaaacaaaa attgcaccct |
| 2221 | agatgtaatg aaaaatttaa tgaatgttaa aattattcaa tattgttttt aaattaatta |
| 2281 | aaaataattt aggcataaga gaacataaag aatatatcat aagaactcat gcaatggctg |
| 2341 | gtcagcttaa gaaatcaaac aaattcagat gaagccaata gggtaatcct ttcccaattg |
| 2401 | cttctctctc tcttacccct gaagtaatca ttattaactt gattattata attaccttgt |
| 2461 | tattttaatt atatataatt atatatgtat agttatatgt attacatgta tgtatatata |
| 2521 | gacacataca tacatgaatc cggaagaaat aggcaaattc ttagcccttc tcactgtatt |
| 2581 | aaatgataaa cctgatacag acatcatcat ttctttcctg aataattgca acaaatattc |
| 2641 | ggaggtcttt cttctttgac cctttttctc ttcagaatat tctcagaaga gtaattagga |
| 2701 | tcatcctttt aaaacttgag ccagactgtt tcattcctca cctcaaaacc ctccaatact |
| 2761 | gtcccacacc atgttgtgag agttcttgca gtgttcaaca aggcttctat tctctctctg |
| 2821 | acctcatcac ctaccattct ctccactgct cacttcacct gggtcatact cttcctgaag |
| 2881 | gtgctgggca cacttctgct ggtcagccgg gaacgttatt tgtaggattc acacagctct |
| 2941 | ctccctcatt ttcttcagtg attacccaaa tgtcattttc ttgtgaggcc tttccggata |
| 3001 | atattatttc caagattttt tttctttttt aattgcagat gcccagaatt gaattgcccg |
| 3061 | tattcatcaa ttcacagcac acaaccacac aattaacatc tgtctctgcc agaacctgta |
| 3121 | tttgtttggt actttgttta gtttatattt ttccctttta tcttcctaac tttatcatcc |
| 3181 | tcattaccct cccaaccaaa ggcaaccagt cttgagtatt tactgtattt cttttcaatt |
| 3241 | caacttctat gtatttgttt acatgaatgt ttacaaaaag gtatatctgt gagaatgtct |
| 3301 | agtttggctt tctatctatt tattctgaac taaaataata tcatgctgtg aatctcatta |
| 3361 | tttgtactca tttatttctc aaatctatat ttttgaaatc tattgatttt attatatgta |
| 3421 | gatctagata atttttttct gacagttgca tggtattcca ttacctgcct ataatatatt |
| 3481 | ttatgtatct gttcctctag ttatggagaa ataaatatat tgcctccagc tttttacaac |
| 3541 | cacaaaaaac tggaaaacat ttctttacta atgcctttgt aaatgtttac ttaccaaata |
| 3601 | atttaagttt ctttggtatt attttatatt tcattttttc ctttagcatc acaaaacaat |
| 3661 | tattctttat tattccagga cagtggtcct caaccttttt ggcagtagag actggttttg |
| 3721 | tggaagacaa ttattccatg gattgggggc tgtgggggat ggtttcagga tgaaactgtt |
| 3781 | tcatctcaga tcatcaggca ttagattctc ataaggagca cacaacctag atccatcaca |
| 3841 | tgcgcagttc acaataggat ttgcattcct aagagaatct aatgccactg ctgatctgac |
| 3901 | aggacgcaga actcaggagg tgatgctcgc ttggctgcca ctcacctcct actgtgtggc |
| 3961 | ctggtttcta acaggccaca gaccaataca ggtctgtggt cctagggttg gggacccctg |
| 4021 | tactagaaga aataaagctt tcaaagttat aagctaagaa attattctat atatttctat |
| 4081 | agcccactat cttcccatct cccaatcatt tcagctcata gttttgcaaa agagatagaa |
| 4141 | cttaaaaaga tcattacaat tacagggtga ttggcgcaat gatggaggca ctgacaggag |
| 4201 | caagagaaaa cttccttaac agagccagat ggagagcgcc cacagatagg ttcttgaaaa |
| 4261 | agtaagctga ggggtgggtc tcaaaggatg aaaaggtgtt ttccaggatc aagcattttg |
| 4321 | ggaatgggtg atcatattaa gcaaagagaa cagcaggagc aaacttaaca agagaaactg |
| 4381 | cagagcctgt gtggaagact caacacaatt ttgatataac cagatgtaaa ttgggagaaa |
| 4441 | ggacatgtca ggagataaac tggagaagta gaaagagggg ctggttgtgg taaaacctag |
| 4501 | ctagagttat atggttcatg aggtccacag aaatatttta tgctagagag tgacaaagtc |
| 4561 | agattagata gtggctgtaa aagagatgtg aagatgatga taattcatct tcaaaaactt |
| 4621 | ctaaggtaga ttaggagatc agtgtggaca ctaggcattt cttttggtga aatgtgatgg |
| 4681 | aaacctaaag ttaggcagta gttagaatga aaaaaaggaa agagatttaa gaaatcctga |
| 4741 | agaggtaaaa tctgcaagga tttggcaatt gcctaaccgt ggaaatgaga cagtgattag |
| 4801 | aagaataatg taactaaaac caaacctgag atataaaata caggaggaga agaaatttac |
| 4861 | aggttaagag ccatagtatt ttcttctatc ctctagcaaa acaaacaaac aaaactacaa |
| 4921 | tctgatatat ttttctatac caataaattt gcatatttag agttttaaat cttaaaagtt |
| 4981 | tgtttaaatc gttaacaaat tattcttaat tagttaaaca attatgttta aggaacataa |
| 5041 | aattgtatat ttccatatct ccatattatc atgtttgcca ttacaacaat tcaacctaga |
| 5101 | ttgattctag atcgtatttt gcaaaatgaa taattagtga ctcattaatt aataaagaca |
| 5161 | caactttttg ttttgatact ttaagttctg aggtacatgt gcagagcatg caggtttgtt |
| 5221 | ataggtatac acgtgccatg gtggtttgct gcactcatca acctgtcatc tacattaggt |
| 5281 | atttctccta atgctatccc tcccctagct ccccaccccc caacaggccc tggtgtgtga |
| 5341 | tgttctccac cctgtgtaca tgtgttctca ttgttcatct cccacttatg agtgagaaca |
| 5401 | tacggtgttt ggttttctgt ccttgtgtta gtttgctgag aatcatggtt tccagcttca |
| 5461 | tccatgtccc tgcaaaggac atgaactcat ccttttttat ggctgtatag tattccatgg |
| 5521 | tgtatatgtg ccacgttttc tttatccagt ctatcactga taggcgtttg ggttggttcc |
| 5581 | aagtcttttc tattgtgaac acacaataaa catacgtgtg catgtgtctt tataggagaa |
| 5641 | tgatttataa ttctttgggt atatacccag taatgggatt gctgggtcaa atggtatttc |
| 5701 | tagttctaga tccttgagga ataaccacac tgtcttccac aatggttgaa ctaatttaca |
| 5761 | ctcccaccaa gactgtaaaa gcattcctac ttctccaYat cctctccagc atctgttgtt |
| 5821 | tcctgacttt ttaatgatag ccattctaac tggcatgaga tggtatctca ttgtggtttt |
| 5881 | gatttacatt tctctaatga ccagtgatga tgagtttttc ttcatgtgtt tattggctgc |
| 5941 | ataaatatct tcttttgaga agaagacaca acttttaaaa gcaggttttt aaataactca |
| 6001 | ataatttcat ctcattctat tgttttaacc catgccacaa ttatttattt ccaggagcgt |
| 6061 | tttagtagaa tattttattt aaaatatatt taaatacttt attaagaaaa ctatgaacaa |
| 6121 | gaaaatgatt aactttagtt ctacaaaagt ttaaaatatc acatgacgtg ctagacaaat |
| 6181 | ctcaatctga gactgctaat ccatgttcct aaagtcattg gaaaatgaca ttctggaaat |
| 6241 | agatcatatt cagagctcta aaacacccat aaaccaatga gcaacagctc atattaattt |
| 6301 | tactcagata aactcacggc tgactattta aaaaaaccct gtaaattggc tgaggaataa |
| 6361 | cacagcagtc tcaacatcaa actaaattgc aaaatttgac ctaaattttg acctagccaa |
| 6421 | gtcatcatta attaaaagga aatgaactca aactatcaga ctatcaaaag taataaacac |
| 6481 | ttgaagaaaa agctgtcttg aaaataaatt gttgagaaat agttcctgtt catttggcct |
| 6541 | cttgcagtaa ctgaagtcat tataaactac cttttccacc aactaaaata gaagtcaaat |
| 6601 | agagcatgcc tctggttagc taaacaczaga accatagaat gtgaaatgga ctcaggcact |
| 6661 | tcagcactaa aagatggctt caaccaataa gtgtatttat ctgatttaat ttaatcaatc |
| 6721 | tgatttaaat ttataattta tatatttatg atttaaattt aatatatatt taatatataa |
| 6781 | ttaataatta tatataatat ataatttata tataattaat aatttatatt agtatataaa |
| 6841 | tttaatatat ataattgata tagttatgat ttaaacttaa taatctgatt taaaaagcat |
| 6901 | taatgaatga aataaatatt ttttaaaaac caaaataata actcagttaa ctgccttact |
| 6961 | ctagaagatg tagtattttg ataaattaca tttcttctgg ttcaagaaat tccttaatgt |
| 7021 | tctcagcgaa ctttgtgaaa tgcatatagc aacatttaag tcttcccaaa atagaattgt |
| 7081 | aatttctttt gattaagcct gcttttcata ccttatggta gcattctgaa tttctgatct |
| 7141 | ctttgtataa taccatagat gcataaaata aaaagtatta aactaattct cctactattt |
| 7201 | tcaggatatg cccaaaatgt tttaattaaa aacgtcaaat ccttcttcta gacttgtctg |
| 7261 | ccagagattt tctgccttta ctgagagtgc tccacacaca cactcacata ctaatcagaa |
| 7321 | tgtggaagag tcgaattgaa tcaattcttt aagtaacata aaattgtgta tttccataac |
| 7381 | acgtctccat gttatcatgt ttgccgttgt aacaattcaa cctagactga tactagataa |
| 7441 | tatttctgca aaataaatag tgactcatta attaataaag acaaatttta aaagcaggtt |
| 7501 | ttaaaataac tcaatcattc tatctcattc tgttctttta atctgtatca caattatttt |
| 7561 | caggaggatt ttagtggaat attttattta aaatatatct aaatacttta ttaagaaaac |
| 7621 | tatgaaaaaa aatgattagt tatataaaag tttaaaatat ataatgaaat gctagacaaa |
| 7681 | tctcaatctg agactgttaa tccatgttcc taaagtcaca atcctaaagt cacaagaaaa |
| 7741 | ccacattctg aaaatacata atattcagag ctctaaaaca cccataaatc aatgagcaac |
| 7801 | agctcatatt aattttactc agataagctt gcttgtaact gtaaaaaata gttgaatcaa |
| 7861 | ttagattgat tcaattagat tcttaaatcg aagaagcatc aattagaata acctaattga |
| 7921 | attagagtct tccatatttg aaacaacagg tcaatgtcat tatctatata aagctaagaa |
| 7981 | agcaaaatgt ggccaggcac agtggctcat gcctctaatc ccagcatttt gggagactga |
| 8041 | ggcgggtgga tcacctgagg tcaggagttt gagaccagcc tggccaacat ggtgaaaccc |
| 8101 | catctctact aaaaatacaa aagttaactg ggcgtggtgg caggcacctg taatccagct |
| 8161 | actcgggagg ctgaggcagg agaatcactt gaagctggaa ggtggaggtt gcagtgagtc |
| 8221 | gagatcgcac cactgcgctc cagcctgggc aataaagtga gattctgtct caaaaaaaga |
| 8281 | gagcaaaatg caaacattct acctaattca aactccaaat tactctttta actcatcttt |
| 8341 | aaagtactat attgtcttat aaagcatagt caatttattt cttcaaaatc ccgtgaaggt |
| 8401 | ttgcttttca tcatgttcga acagtttccc taacttttaa aatcagtttt tacattgccc |
| 8461 | tctgctgtta acatttcttt tctgtttaat ctttctaaat catacttgtg tttagtgatc |
| 8521 | attttacatg atgaccatta tcacatcatt tcatctttat gatgctggtg cccgaagctg |
| 8581 | ttttatttgc attttgattc tgaatcattt ttagaacaaa gaacctcatt tgtcttccta |
| 8641 | gatcactctc agagctgtct gtcctgcttt tcgcacctct gcccctgctt gcattccagt |
| 8701 | ccttatctat tgaattgcct ccaaacgagt cccccaactt cagataagat cctctctaac |
| 8761 | ccatccttca gaaccccaaa tctgtgtaat tttccttctt aaaatcaatc tatatcttcc |
| 8821 | aattgcctat ttacagtgtg gagtctaagt tcttcctgac agcttcagat atttcaYaat |
| 8881 | ttatctgaac tgtgactgct ctcaaatcct atcttgcact actccctcca gcgcagaact |
| 8941 | atgctacagt caggcaactt gtccacaagt tcaccaatat cacgtatttt ctcatgttgc |
| 9001 | aaaggctttg acaatgatgt ttccccatat tggaatcctt gccagcattc cttctactat |
| 9061 | gaatacatat tacttttatt tcaagaacta tttaaaaaaa tattcctgcc aggaaggctt |
| 9121 | ttcttatcct tcctacagca gagtacctta atattcaata tacattcaca tctgtcagaa |
| 9181 | ctctatgcca attgttcatg aacatgtttg tctgtcctac tacactggtt ctttttggca |
| 9241 | gagacactgc attaataatc ttctgtagat ccagctgcgc ttgtgaaaag agaagcacag |
| 9301 | tcaatataaa ttcagagtta ctatactttt aagtaaatta tgtagttttt aattgaagta |
| 9361 | ataatctctc tgagttcagt tttagggtgg taaactcctt aaaacctgta taacaacata |
| 9421 | caggattaac aagacttttc aaggtgtctt aggtatggca ggcatatgca aaaatctaat |
| 9481 | gttttaacac actctctcaa tgtttttgtg taaacattta tttcagtttc gcaattatgt |
| 9541 | tattaaggtt ggtttagcct ttggtttctc tgagttttta ttacttgatt caaacatatt |
| 9601 | tatcaagtgg gcactttgtg caagtccctg tgccagacac tgggaatagc ataaatacgg |
| 9661 | taatacctta ttataaagga ttcctgtgag aattgggttc accagatttt ttaaaggacc |
| 9721 | tagccaaatg cttggcaaat aaatgaaagt tgtgttgttg ttattgttat taaggggttt |
| 9781 | gtgtagattt ttttaaaaaa gatgaatcat caattagaat gaatataaat aatcattcaa |
| 9841 | tatgacaata tcataataat atgtgtaaga taaagccctg ctactgctac ttagcattcc |
| 9901 | ataacaccaa caatagttgt ctgggctcca gagtgcaatg taaattagag aaatcacaat |
| 9961 | ggtgtgctat ttcttggata agtgagcatc cactttggat gataacagcc taaaattgca |
| 10021 | tctgcttagt gcaggacaaa acatgacttg agtagatagc aagtcctttt tcaaagtaaa |
| 10081 | agatttaaac agaataatcc acctccacag tttttagtgg tacaaagcaa ataatagaat |
| 10141 | atattgatga ttttagagat tagcagtcaa catttaataa acaagctgtg ttgtgctttc |
| 10201 | ctcagagtct attatctcgt gtccatagta ctttcttatc tgtaaaactg gttccttaag |
| 10261 | ggaaagtaat acttggttga ccaagggctt cttttctgaa atgtgctggt cttttctttt |
| 10321 | tccagtttta cttttttact tacctataaa atattttctt atacagaaaa ttatgtaaac |
| 10381 | ttccaggatt ttctagatat aataaatttt aaaaaatttt cattaaagta ataaactaaa |
| 10441 | aggtgggaaa gtgaattatc ttcaataaat tttgtcattt ttaaaaattc tgaatttttt |
| 10501 | cttcctgaac atttataaat ttatttttaa actttcagtg aactggaaaa agaagttaaa |
| 10561 | agttaaaaac tttgaatttc atagactttt tttcctccag agattttgta ttttacatca |
| 10621 | tcaaagtcag gttttttaaa agtgaaaaaa actttctcta ttcaacaatg aagctatcag |
| 10681 | gaggtcttta atgagtgtgt cagataaaaa gagtacttaa ttgtaatacc tactactgaa |
| 10741 | gagttttaag agaatggata ttcataataa tttaggggaa tggtagaaaa ttatttactt |
| 10801 | caaataatac catttttttc ttcttattat tatgcagttt gattcctatt gcttgttttt |
| 10861 | catttcttca tataacaaaa ggttgaataa ttttatggct cttttttttt tttgacaagg |
| 10921 | tctcactctg tcatccaggc tggggtgcag tggcatgatc tcagctcact gtaacctctg |
| 10981 | cctcctgagc ccaagcgata ctccctcctc agcatcccaa gtagctggga ccacaggcat |
| 11041 | ggcgccacca cacccagcta attttcgtat tttttttaga gatggggttt tgtcatgttg |
| 11101 | cctaggcttc tcttgaactc ttggactcaa gcaatctgcc cactccagcc tcccaaagtg |
| 11161 | ctgggattac agggtgagcc acctcacccc accaccattt tatatttttg acttcttttt |
| 11221 | taaaaaaata aaatctcaca tctcacatat ctcaacccgt tattttaccg aactcatagt |
| 11281 | caaattgcta agagaaaccc actgccaatt atttttttta aatctgtttc ctgggttctt |
| 11341 | tacattcata tttcttaaaa taattttgta ttgcctttat aatatatggg ctttttaaat |
| 11401 | tttggtatat gaagatttag cttttctcat ccttgtcata tattacgatc ttggttaaac |
| 11461 | taacatttgg aatttatatt aacttgcctc tttaatattc atttttgaga aaatttggtt |
| 11521 | cttatatggc aagctttact taattttctt tttttcctta agttattaat tgcttttttt |
| 11581 | ttttacaatt cgctcagctt tctgcgtacc tatctctaac tgttctcaac acttccaaga |
| 11641 | gcacctcttt caatactatt tttcacttgg ccagacacat catgaatttt gtcaatgctc |
| 11701 | ttctctcctg catgagcacc ccattcactg cccacgtgca gcctagaact ctccatatat |
| 11761 | ttttgttttt gaaattcgtg ttatggtgga gatgtcaaag acaaggaaga gtcacattct |
| 11821 | atgccagtgc tatccctggc tttgaccttc ccacctgggg tttttaaggg tagtacctat |
| 11881 | gtgtgtggcc gacgctcagg tgaagatgca aagcagtatc agctctggtt ttgagtccca |
| 11941 | gccaagttga tggacactct gccgcagttg cacccatatg ttgagaaaac aaaactacag |
| 12001 | gctggctctt ctgctagtgc ttggtcacag tgtataccag ttcacctccc tggagacaaa |
| 12061 | cgccattcga aattgggttt tcagggccct ggaagtattg aattaatctc taaacttctc |
| 12121 | tagtttctgt ggagggccaa ttttttttat tgggctacta gaaatgtctg taccccaaaa |
| 12181 | tacatgggaa tcaaattcag atttttaata actgctaatg aactcttcat atttgatatt |
| 12241 | tgatctccac atttgatatt tgatttccat atctctatat ttgataagat tttctcaggt |
| 12301 | gttctttatg cctgctgcat ggctattgtc tttagattct ccttatctca tcatggaagt |
| 12361 | cctctattct ttgtgtctct ggattcacaa agtattgtgt gtggggtcag ggtatgcttc |
| 12421 | tatgtggtta attcgctatt ttggtggagc atatcttcaa ggagcttcct gagataggta |
| 12481 | gcatggaaag taaaatgttt gtacttagcc ttctcaaaaa tcatttcctt ttacccttac |
| 12541 | acttgatgga tagtttgtgt tagattttta gtttacaatc ccttccattt aaaatttcaa |
| 12601 | aggcattaat tattatcctt ttctttttaa gcctctatta ttgctgttga gatatcgttt |
| 12661 | gaattcttat cttttcttct ttgtagttaa cgtacattaa ttaattatct ggagaaacct |
| 12721 | ttagcatctt ctctacgttt tctgttttct gaattattat gatgtctcac gctgcaagtt |
| 12781 | tatttatttc ttgtctttct aatgtggaga tttgttttct ttagtttaaa gaaatgtgtt |
| 12841 | gtattttgaa taacgtttct tttcatctct cccttttctg tggaattact gctagttaga |
| 12901 | atttgagcct gataggttaa tcacttaatt gttttttaat aattataagt ttttttgttg |
| 12961 | ttattgctaa tttctcttca gtgaaaagcc attgttactg acatgctagt tcataaattt |
| 13021 | tttttctttt tttttttttt tttttttttt ttttttgaga cacagtcttg ctttgtcatc |
| 13081 | caggctagag tgcagtggca cgacctcggc tcactgcacc ctctgcctcc tgggctcaag |
| 13141 | cgatgccccc tatcttagcc tccctagcag ctggaaccac aggcatgcgc taccatgccc |
| 13201 | agctaatttt tgtatttttt gtagagacgg ggtttatgcc atgttgccca ggttggcctt |
| 13261 | gaactcctga gttgaagcaa tctgttctcc tcggtctccc aaagtgctag gactagagac |
| 13321 | atgagccact gcgcctggct ctccagttta taatttttga ccttgtttgt tcttgtttgt |
| 13381 | ccttttttga gcaaaacatg ggagatcctt atttagtata gtgatcattt agtgatcata |
| 13441 | caagttacaa tattttaatc ttatttatca tttgctcatt gcttttattt ttggtttatt |
| 13501 | ttactatatg tgttattatt atttatagac aaaaatgttt attatttctt tgaagcctta |
| 13561 | tggcttctta tcttgcttag gaaattgccc ctcatattcc actaagcctg tacaaatctt |
| 13621 | acttggattt ccttcaaaca tttttattat tccattttta aatttaatct ccttaattaa |
| 13681 | cctgaaaatt atttggtata tggtaatgac tagaagtcta attttctttt cttactgaag |
| 13741 | aaaattatgg cagaactatt tgataaacca actttttcta tggaatgaaa tatctgttcc |
| 13801 | attggaattt ataataatca tttactttaa aaccatatat ggactggtta agttatccat |
| 13861 | ttgcccattt atctatttgt ctattttgtg taatttaatg ttgttttgaa tacagaaatt |
| 13921 | tttaatatct gataaaacaa gatttttaat tttggcaagc tacatagcac ctgtaatgta |
| 13981 | aatggtgtta ttagttgatt gcctatcatc attaagtatt ggatgttttc ctgcccaaac |
| 14041 | caattaagat agtcttatga atttaaaatg ttttttcatg aaatagctct gtaaccttca |
| 14101 | atatcaaaat aaaaatgttg gtaagtacaa tggtaagagt caggtgatgt tagattagct |
| 14161 | atctgtagat tctaactctt tttaataagt tgactataaa actctgcttt gaatggtttt |
| 14221 | actgctctag ttctctgttt tcttgactat ataaggtgga aattattatt tcaattattc |
| 14281 | catatctata tctcctactg tcttgtgaca aatgaaccag acaatgtgaa ggcatttaga |
| 14341 | acaaaataaa cattatatat ttgtgatatt tacacatctt ttgttaattt tctacatcta |
| 14401 | tactaagaaa atattcttgg cttatattcg ttgtgttatt tgctaagttt tagtattctt |
| 14461 | atcttgattc aaaagcctct ctctcataag taaatcagag caacaactcc cagtgcaaca |
| 14521 | tttcagagac ttctaggtaa agaagacatt tcaaaaaata ttgaaaacta ctatttcaga |
| 14581 | tattaatctc tgaaagtcta aaatattctg ggctttcatc aagcaattga caatgctttg |
| 14641 | atgtgtgcta agacgaagaa atacccaaga tcgcttccag aaatttaatt catttaactt |
| 14701 | tttagtggtc aatcgatacc ctccagagat gaatctggct tagatttaag acacagattt |
| 14761 | ttaaaaaata tttcaataat agggtatttt gcagtaaatg ccattaattt taaattccca |
| 14821 | gtgaataatc caggactaga aataagtgac actggatttt agattcacgt aatttaggaa |
| 14881 | aatgtgataa ggtgataagg tccagggaaa tcacacccca aataggagga ggctttcccc |
| 14941 | ttgcaccctt tgctctagct tcactgaata acttctcttc tatatacccc caatcttttc |
| 15001 | ttccatctgt gatgttcctc atggtgttct tcagtctgaa cttggcctca ttcattgcca |
| 15061 | catgtctaga ccttgttcaa ctttcaaagc agtcactact tgtaacctct ttcacaaaac |
| 15121 | catgctcccc accatcactt ctacagatgc tgccatgctt ttcaatgctg aaattccaaa |
| 15181 | gcatataatt ttccctctgg cttatatatt tttattgtaa ttagtcattg catcatggtt |
| 15241 | attaaagccc cttctcagga tgattgttaa aatatacaat aggtgtggta cagtcaccaa |
| 15301 | ccaatctgaa tggatcctgg ctgtacacac ctggttcagc aagcatgctt actggctgct |
| 15361 | gatgaaatag aaccagccat actagatgga cagtatgctc cctcagatca gtatatgtac |
| 15421 | tgaattgatc tctgtaggat ccaaagtttt aatatagtgt tttgcatata atagatacac |
| 15481 | tcatatatag cactaatcaa ctttggaaat ataagaaagg tagcctaagt attgggaaaa |
| 15541 | ataagacagc aaggtcacat atttgtggag tgaattattg gatgctataa taatagctgt |
| 15601 | tattatactc taattgagga tcttccataa tggtttcata ttttctcttt gagagtagag |
| 15661 | cctagcaaat ttctagaatg tttgagattt atttgttctg gaaactgaat ttccattctt |
| 15721 | actgaaacta aattggattt tataaagata cttaagaaaa taaatgattt attaggggta |
| 15781 | tttgttcttc aaaggtatac agccaagaaa attaagcttc atggaaagtt gtgattgtaa |
| 15841 | tactttttaa agatgataaa cacctggttt aggagactta atggatcaag gcagctacta |
| 15901 | catggatgct cactggcaaa aaataactat taaatacatc catcttaact aatggaaggg |
| 15961 | tcagctagct taaaggatgt aattagatgt ttttagtgta aattgggaaa acactcatct |
| 16021 | gtacaatttg aaagtctaat tggtttgatt taaatgttaa agaaaagtgg tcgtgtatag |
| 16081 | ctctcattgt caacaagtgc acaatatttt atcaatcctg aataatgatc atttctttgg |
| 16141 | atgggatcaa aatgactctc acatatactt ctaatttaat aatagctaca acttaaagga |
| 16201 | tgttaactgt atgccaaata caacacttat ttttgcttta catatgcact taattaaatt |
| 16261 | gtggaaattc aatgattgat tagtggagat agtctgaaaa agtattactg ttttatacta |
| 16321 | caacttaggt tgaatcccag aacccttcat agggctcaca ttctgaccca ttgaactaat |
| 16381 | ctcatgctgg gattaaaatt cataaacaac ttctgtgaat aaggtaaaac attattagag |
| 16441 | aaaatgacta agtggaaaat tattgttttt aaattagaag cagaataaat ggacttgctt |
| 16501 | aataagaata acaagaacat ggctatttaa cattaaaaat actcaatgac attctcaaca |
| 16561 | gaaattgtaa ctccctaaaa cattactttt tacattctga accccataaa gcaaaaacaa |
| 16621 | gaataaaaat tgtatgtgaa caaaccgtct aaaagtgatt acttaaaatc tatgatatac |
| 16681 | tctggattaa agtatgctgt cctaaagcaa atctttgtag atgagtattt cttagagtgg |
| 16741 | actaaggtat tcataaaaca aagtgcttaa atgaataata taagtaatcg agatgaagtg |
| 16801 | gaacatttca aggatctata atttcaagaa aactgtgctt atctacagag tcacagtttt |
| 16861 | tctgatgtcc tggtgaagga ttgaatggag gaaaaataca ataaaagaac tatatatttt |
| 16921 | gtctaaaaaa aattgaattg actcgtttgt ttttgttccc ccgctaaatt ggctgtttat |
| 16981 | gttcactgac aacaaaggca aataaagata attatttgga tcactcaaaa caaacaaaac |
| 17041 | actgattaaa catatatctc aagtacaaca caaagttgat tccatatttc tacgtggaca |
| 17101 | aaacaaaatc ttgcactttg cattgatgtg tcctcctatc tgcatagagc aaatacattc |
| 17161 | ttaatcattt tctcctcact ctgtttatat attctttcta atttaaaatt gcctctgaaa |
| 17221 | gtctaatatt attgcagttt tcttctccat attattaact ttttaaaata ttttgcttat |
| 17281 | agtgattcat gcccaattca tatagataYg tagattataa atttgattat tgtcaaatgt |
| 17341 | taatagcaac tcagtgaaaa acaacttgtc caaaatgttg ctaccaaaag ggcacttgac |
| 17401 | tataaataac tcacattata ttacatattc tgtcttgttc ccatcYtcaa cccccgcccc |
| 17461 | tctgctcaat aactaatcta agatcaactg ttgatcaggg tcagttcgct aaaggggaaa |
| 17521 | catattgact cataagttta aataaaaata aagtcgcctc tgattggctc cttcttggaa |
| 17581 | agcaagcaag ttttgtgtgg gactgatatg aaagaagaaa gcagtattag gatgaataat |
| 17641 | ttctgttcat tgacagctgt taacatttat actaaaacaa ttttatttat aaactttagg |
| 17701 | attaaagaaa ggtaaaatta agtagctaaa actatgcatg aattgcttca ctatatatct |
| 17761 | aaattctaga ctaatacgaa aatcaatagc tgctaattgt atagatgatc atatttgtta |
| 17821 | ttcatttgct caacaaacat tctctaagca actctgtggg caaggctgtc tgctaagtgc |
| 17881 | tgggatacca agaggaataa gcgctatctc tggtttgaga gttgttcagc cttatggagg |
| 17941 | agacaaaaat agaaataggt aatttaaatc aaagaacaat gataaaatat gcaaattctg |
| 18001 | tcaactgtgt tttaagagca tagtaggaaa aggaggcaaa attggggaga gggtgaatta |
| 18061 | agtgcagggc atacgtagct tgatgcatcc atgtaaaata ttgtgctggc cctatactta |
| 18121 | tatgctctca gatgcattct tcggtctctt ccttcctttc ttctcagagt agctgccctg |
| 18181 | tacagttttc cagacctctc cgtcaactgg cttccagttg gttttgatca ggcactggca |
| 18241 | gagattggag aatgggagga agagagaacc aggatattte tccccatcct tgtctgtctc |
| 18301 | aggatatatt tctggcattg gctgaatttc tttcatgttt cttgctccca gaaagcaggc |
| 18361 | ttgtcctggt ttcggctctc gctgtcctct agcttatggg gacaatagct atttttctgc |
| 18421 | tgcttcacta tcccccactt agtttctcag cttctttcac gagtatatca tcaattgcta |
| 18481 | gtcatttgtt tcctctattt caaatattca gagccatttc tagtttcctg gttggacaca |
| 18541 | aatgcctgac atactgagtc taaattccag gagtttgggc agcaggtttg ggactaatca |
| 18601 | acatataaga gagagttcat gagtgtacac agagatacgg taggtaaaaa gattggtaga |
| 18661 | ttgagggtgg aaccctgaag tatatagcaa gagataaagg aaacatagct tttgaaggcg |
| 18721 | tccaaagatt aatttacaat ttcgaataga acaagaaaaa tgtggcattc acttaagccc |
| 18781 | ctgagaaaac ctaggtatca cttagaccca gcatttgtca gatgaggttt ctgaagRtag |
| 18841 | agaaattagg tcacatatct aataaagacc agaattgaaa cctaaacctg tttccctatg |
| 18901 | tagtgctctt gtgataacac acccattatc atggtaatag tgtgacattt ggtgtcattt |
| 18961 | tcacttgttc tgtccttgtt ccatgtgtgc acttcagttt ccttttatat aaagccaata |
| 19021 | tatgttatta gttacctcat aggtaattgt aagaattaaa tgagacagtg catgaaaagt |
| 19081 | acttagaata aactacttag aatagttaaa tgtatagaga gaggttggaa tgacacatga |
| 19141 | aggactatag ggctactaKa ttttatttat tttatttttt gagatggagt ctccctctgt |
| 19201 | cgcccaggct ggagtgcagt ggcgagatct gggcttactg caaactctgc ctcctggatt |
| 19261 | caagcaattc tcctgcctca gcctcctgag tagctgggat tacaggtgcg tgccaccacg |
| 19321 | actgtctaat ttttgtattt ttagtagaga tggggtttcg ccatgttggt ctcgaactcc |
| 19381 | tgacctcgtg atctgcccac ctcggcctcc caaagtgctg ggatttcagg cgtgagccac |
| 19441 | cgtgcccagc cagggatact agattttatc acagtattat gaaaggtaag cttgtcatcg |
| 19501 | taaagcttat agatactcct tttatatgtt tgctgacgtc aaggattttt cacatttggt |
| 19561 | aataatttat tatttgttta ataaaacgaa atttgaactt aaatttggtt tgtctttaga |
| 19621 | agttagttta gcaattaatt tttctgtaaa atgattttta caccattaat ttcttctcag |
| 19681 | cttcgtatat ttaagaattt ctatggactt aaatgaggtt ccatgattga ttttctattt |
| 19741 | gcatgatatt ttaaacaaaa tatatgttat atttttaagt tcaagatttc cctcccaccc |
| 19801 | tgggaaagtc ttctgagtct ttttcatgtc atccttctca tttttctttc tcttttaatt |
| 19861 | tttcagtacc ccaagttact ttaatgttaa acacaaatga gaaaattggg gaattgtaaa |
| 19921 | atgaatacag tcattctcat ttaatagata atgatatctt actacctatt gcttttgcca |
| 19981 | ttagtttgta acatttaaat tcatgttaaa agaaaaacaa ctgaaacact tatactgtag |
| 20041 | aatgcagatt cagccaaaat ccaatattgt aaatttttac ttaccttcag tgtctgctag |
| 20101 | tcctactcct acccctggct gttttacagg ctcttgtcca ttctgtccca tcttccctct |
| 20161 | gtttagaaat aaaataaaat gatatattca gttatgatct catgaatgca aaagaggcta |
| 20221 | aatttgcata aataaaatga cattgatagt aatggcaaaa cctgcaatta cttttgcaca |
| 20281 | accgaatatt tttggacttg caataattat ttgaatggca catacataca cagttaattg |
| 20341 | ctctatatca taattttaaa atgtgaactt gtttattatt cctattcagg ccaaacaata |
| 20401 | tacctcaaca ttcagtagtt tttatttgta gactatttat caaaaaataa gaccatcatg |
| 20461 | acatttagaa attttgtttt agaatttttt atttatttct aactcaacaa tgagaagggg |
| 20521 | ctttatattt ttatatgttg tactgatgag ttattccagt ttttttggta aaaagagcaa |
| 20581 | tattgaatta aataatatat aaaaatgata ttttaataaa atatttctta tttcatgtat |
| 20641 | ttttttttca actcctttct gtgttttcca ctgtgttgag caaactggct tttgtagtca |
| 20701 | ggctgcttgg tctctgtttc tggctttgct atttcttgct atgggtaatg taaggcaaag |
| 20761 | tacctggtca agactgtttc tccttttgtg atataaagat aataatatta gctacatttt |
| 20821 | gattatgaaa aaaaacacat gaaaaatgcg tgaaaagggt taacagagtg cctgagaaat |
| 20881 | aataagcatt caataaacat gRcatattaa caggtaggca tgStcgtagt agccttagaa |
| 20941 | ttcaaccaaa atctgctaat cacttttagt tttcagcttc aaactttttg gaaagtcaac |
| 21001 | tagatgaaga attcaggata ctttgaggac tgtaagctta gaatgttaat aatttaacag |
| 21061 | ctaaaSgaca gtagaccact tcttatttct tcggctagag aaaaagctac atggcacatg |
| 21121 | aagaagtttg gaatttgcct ttatatRtgc ttagataagg cgttctaaaa agatggaata |
| 21181 | aaaaattgtt acagaaagat gcttgaacaa aagagcatct tgatactgca ctttgtagac |
| 21241 | aacacttcta gcatagtggg atttactaac aattaattat aactttcata ggaagtattt |
| 21301 | ggtttgctag acctgctgaa aatgtaacca tatttatttc ttaataatcc aattatatct |
| 21361 | tacttgaaaa ctgctctaaa ggtcctctta ttctacttca gagaaggtgc tgaaatgttt |
| 21421 | ccgcacataa taaaagaaca catgtaaatt cagaaattat tttatctcaa aagccattac |
| 21481 | attgttccat tataataatt ctaggttaaa ggtttaagta caaattttta ttaagtgtgt |
| 21541 | atgtgaggga ccaagttcat gcaaattaag caatttccaa catttccatt ttaccatata |
| 21601 | tatgttaaac catggtctgc atggtcaaca tctgcataca tatttaagta acacacaaaa |
| 21661 | ttttagctta tgtaattgct tcatcaagat tcaagacaag tagttaagaa aaaaaaaaga |
| 21721 | actctgctct atctgttagc attagagggg aaaaaaagct taatagcagt aatcttactt |
| 21781 | ttatcagtac gctacactgg tagagtgaac caaaacatac cttgttgctg agccaaactc |
| 21841 | tattgaaagt gcaggtcttt ttagtattgg taatctagta gatcagaata ctaaaaaata |
| 21901 | aatattctta tactatccca gacttttaaa aagtagttac tgtaagtctt agtaattgta |
| 21961 | gtacaagtac attttgataa tttttggggt tgttcttgag tcttagactt tatatgttaa |
| 22021 | tggtgatacc cagccagtct ttgctttcaa ttgaaactga ttttaacata ggtacttagc |
| 22081 | caatatgtgt agtaagtaca ttttgaagat tatacaaaaa actttcatag gattgtgaag |
| 22141 | ctaaattaag tgcaaaggaa atgtaactgt gagtcgtgtg aaggatgtgg tttctctgaa |
| 22201 | ttcccaatga ctacacttta gctcagaaca ttctggctat acattttata atttatattt |
| 22261 | ttaaataatc gctagatatt tattaaacac ttttactagc ccaagctata attttattag |
| 22321 | atacaatggg ggatataaaa gaactgccaa atactacttc ttacacatat cctataatgg |
| 22381 | ttttcaaaag taaatattcg ttgtgctcat atggcaaagc aagagtctcc tagacagttg |
| 22441 | tgagaagctt tgatttacaa gagaaacagg aaaatgggaa aaatcaaaat tagaatgtgg |
| 22501 | tatatatata aaatacataa tctacttata ctgcagacat tcaaatattg acaaaatgac |
| 22561 | atatttactt tttggaatta taactttacc atcagtgtta aattatatat tttgaaaggt |
| 22621 | gcagaaaaat tggaaggata acaaattatg tgtggtttaa aacctttcta aattcacatg |
| 22681 | acatttacag cttagaaaga aatttaaaga atttgtagaa attcttttct gtcaaattgg |
| 22741 | atcatcagaa gtcaaagacc aatgggatta gatttctttt aggaagaatt tccaataaaa |
| 22801 | tataactgat taagtacaaa taactctatt gaatccatag actaaatctc ataaaagaaa |
| 22861 | agaaagctaa gatggacagg tgatacaata agaatttgct taatttaaat tgaaaaaaat |
| 22921 | gtagttcaaa acaacaaaat gaactcgttt ttcactgtaa aaatctattt tcactaatgc |
| 22981 | tttgctttta taaactgaac cattatcaaa atattataat tgttgtggac atcagaagga |
| 23041 | aagatttata agaaccttta ttttaaaaca aagagaaata gacaatccac cccctgaaaa |
| 23101 | gtcttcactt agcattggag aaaatgaaaa agaccctttc acaacccagc tcagggttgg |
| 23161 | caagctctgc aacgtgccag ttagtaaata ttttagatgg gctgtattct aacgacttaa |
| 23221 | ttctgccctg cagcacaaaa gcagcatcag gcaacacaga gctaataggc acggttgtgt |
| 23281 | tccaatacaa cttagtttat attcactgaa atttcaattt cctataattt tcaagtgtca |
| 23341 | caaaatatca cacttctttt gttttttcct acactttcga aaatggaaat gccattttta |
| 23401 | gctcacaggt cttagaaaac caggtggtag actgcttttg ccctggcgta aaccctggga |
| 23461 | cctgggtggc ccagtggtat tttcaaaatc cagaagacta gttaatgggc aggaaaagga |
| 23521 | aaagtcagga agtttggatt ttgttattgg aacagacata atctacatat ttatcttaaa |
| 23581 | tgccaccaat atgaccaagg cgattctaac acattaagta tgctaaagtt ttgcatttga |
| 23641 | tttgcaaaat gacttttttt ttattttgag gcttaacatt atgagtgatt cttattccaa |
| 23701 | gagtacctac tcagggacct atgcactact cggtctttgt tctagaaagt atggaaacac |
| 23761 | ttcgaaacct tcaatgttaa ttgtagaagg atttatagtt gaaaagtctg tatgattatt |
| 23821 | cactatgtcc tctatttcaa atatgatata tggattcttt gaatatgttt aaatatctcc |
| 23881 | tgattcaagg taaattaata tatgtttgtc tcaaatacca gtaatgtttg tatttggtca |
| 23941 | ttatgtgatt aaaacattac tgtattcctg ctgaacttta ttctggcatg gctaagtgat |
| 24001 | tagcagtttt aacatcaaat acagattgta ttgattaagt agttctggca gtgactaaca |
| 24061 | tttatcctta gaattaactg aattatccaa tctttcttga actatctatt tttacatagg |
| 24121 | cattaacagt tcaagtgtct cagggacaat gactcagaat tgggtaatta tccagttatt |
| 24181 | tcctgttaac tgttgagaat gccttactca tgatgctgag taagctttgt agtaacaaaa |
| 24241 | agagacccac ttacatcctc ggaataggaa atagattagt ctcccctgca gtgctgctgc |
| 24301 | ttccttcact gtccacgcta tacccactgc caaagctgct gcccgaggag ccggtggaca |
| 24361 | cagagctttc tgcaggccgg tgattgggag gctttgctgc atggtgtggg aagattcaac |
| 24421 | accacagtca ataaatgaag ctgacattga tctacaaatg catcattata catcttctta |
| 24481 | atttacatgt gacaagaaac agctactgaa gaactctaca tttttcaacc cgccttttcg |
| 24541 | tttcctttac tgataatgaa tggtggcaac aataaattat tttactgttt ttattggaca |
| 24601 | tcaaggaaaa gcaatcacct ttagggaaaa acacatttaa tttttagacc aagttaaata |
| 24661 | gtatgtaact atgctactat tattattttt gtggtttctt tgtacagtat tgcaaaaatg |
| 24721 | atatggtaaa taaaaagaat tttggctatt tcatggagaa aagaatttag aaagacttgg |
| 24781 | gtagatagaa atggcaagat aaagaaaatt gcttattttt ttcctattac tagactataa |
| 24841 | tctgtttttc tccttagaaa caaaaatgca aaatagaatt ttaagaagtg attgaaaatt |
| 24901 | ctgtttgttt gtatgtacct ctgcctacta tctccagcaa ggttttaaaa tttaaagata |
| 24961 | tttgtttctt ttcaggcatt tcctagttta tcattgagac ttgtctcaag aatttcctaa |
| 25021 | gtttgatatt tgaatataag aatccatatt cccatacaga cattacaaat ttgtgcaata |
| 25081 | aattatgcag taggggtata gtactaatat tgttgtcaga tttgagaaga aattttgatg |
| 25141 | caaaaagaag gaagataagg caagtgtttc cctgattttg ctggggagaa gtttctctag |
| 25201 | taagtttttg aaatcgttga tatttgtctt ccgccagaga gagtgaactt ttcggtcaca |
| 25261 | ccgatctggt tttgactccc aacccactct ttagcaactg tacactgctt ttctcatttg |
| 25321 | aaaacaatgg gagtcagtaa tgcctacctc ataaaattgt gattatgagg agtgagatat |
| 25381 | gtaggagccc agtaacttac tcttcttcct cttgtgatat tatttgccac atcaccactc |
| 25441 | ttctcatggg ctttactgtc aacagctgtt tcttactgtc tttgagctta aatagtcttt |
| 25501 | gacaatgaat ttaactctat tctcttctca aggtatcccc actcttctca agggctttac |
| 25561 | tgtcaacagc tgtttcttac tgtctttgag cttaaatagt ctttgacaat gaatttaact |
| 25621 | ctattctctt ctcaaggtat ccccactctt ctcaagggct ttactgtcaa cagctgtttc |
| 25681 | ttactgtctt tgagcttaaa tagtctttga caatgaattt aactctattc tcttctcaag |
| 25741 | gtatccccac tcttctcaag ggctttactg tcaacagctg tttcttactg tctttgagct |
| 25801 | taaatagtct ttgacaatga atttaactct attctcttct aaatctagat gtgttcaaac |
| 25861 | ctacagttct acttagagtc ttttttcttc ataaatgcgt ccctatccca ttaggagttt |
| 25921 | attagccagt tggatagagt tatactaatg ggtgaacagt tgataatcat ttcacgatcc |
| 25981 | taagatatct acatatttct taaggatatg tataaaaaca caattattct taatctggtg |
| 26041 | gaattttaac caatataata accacatgaa gtgaaacaca taaacattgt gtgggaaacc |
| 26101 | atttgaggac aggaaatcca ccctcagggt agctaggatc aagtcttgca aaggatttaa |
| 26161 | cttttccccc atttcttcac tttttcaatt tcgtcggtag caggttctct aaagccacaa |
| 26221 | ggaattttta tatatattcc agtggtagta ggatcccttc ccgagaatag ggaacaagaa |
| 26281 | agaaattgtt aatagttagt caattgtata tcagatgtgt ataagttaga aacggctatt |
| 26341 | taggaaaatg tgttcctaac ttatagacat ctgatataca attgactaat tgaactgaat |
| 26401 | gctgttaaac aaccagtcat caaggaattg ctagtaaccc acaggactgg atttgtggaa |
| 26461 | acagaatagt tatgtttctg aatattttct ttattttatg tagaagaaac ccagatggtg |
| 26521 | tagttagtgt taaaaaaaac ttcttaaaag aataatttta aaaatgtaaa ggtaatgtga |
| 26581 | gtaggatgta aaatgatgga catgtataaa gtagattaaa atgcataacc tacataatga |
| 26641 | gtcagattag tcaattgatt ataaacaaaa gaaagctgtc atactttttg atgggattct |
| 26701 | ataatccaat tcataatttt tagtatgaca ttgaatggaa gacattaatt tcatcaattg |
| 26761 | gaaaatcaca atttactatt ttcaaagata aaccttaatt attagacact aatattcaat |
| 26821 | caatttatat ttattaaatt aaaataattt taattcaaga gcaaactcac aatgaacaaa |
| 26881 | aatagctaca ttataaaaat gatttattat ctatgtttca aaatcgtcca tcctttgttt |
| 26941 | tgtcaacttt ggttttacct gatgcgattt ttagttctat tcctcttctg gaaaagttga |
| 27001 | gaaatgtgta cagaatataa aatagcataa taagaagaaa gaaacaaagc tagaaataaa |
| 27061 | tttaagaaaa gacaggtttt ttttccatct tgccaatctc tttgaataga attctcattt |
| 27121 | tgaaagcctg tgcaagtgtt gtcaatattt taaatgcaaa caaataacat tttataagaa |
| 27181 | atattaaaaa agaaaaccaa atccagggga catttaccta tgatggaatc agtctaacat |
| 27241 | ctctgcaaaa catgattaca tctaaggttc tgtctatgga aattttaggc agttagaaat |
| 27301 | taggtgaata attcaaatga atctagttat ttggagttat cacaacaaaa catccggtgc |
| 27361 | tggattaaat tctgtattct taacttggta tctaagtgta gtgcagagat atatagcctc |
| 27421 | acatttggtt agtttctgaa aaatgaatta aattaaaaca cacaacagat cttttcttct |
| 27481 | tgctgggctg tatctgcata tcataaaatg tgataaaaca aaaaatggag aggttttgat |
| 27541 | aagctggttc taagaatccg tattttaata tatcttttcc agatacttga aacatgtatt |
| 27601 | tactcatttc tttctcttga aacagacttc aagaaataaa acatcaattc tattaaattg |
| 27661 | agctaaatct gcataaaggc aaactttttt ttaacaaata aagtgtattt aggactttat |
| 27721 | tatctgaggc tggatcttag gtaatttcaa gacatctaat aatttccacc acattcttta |
| 27781 | ttttgcctct caaaagagtt ttttattggt ctggtctttc caaatatatc tcttgtaatt |
| 27841 | ctccttcaaa cactttacat ggaagccctc tcagtccatt cactctggct ccctttggtt |
| 27901 | tggctttttt ctttccaact ttactaagct ctcttttatc ccatcacttt cttgtatcta |
| 27961 | aattaaagtt atctgtaaag gcctagctca gaaatcacct gcttcatgca tttttcctca |
| 28021 | ttctcccatt cagaagtgac ctctcctatg tctggactct tgtagcacat actctgtcgt |
| 28081 | tattggaagc aaagcctatg ttttctcctt ggtaacttcc ttaaaagctt gtcccggaac |
| 28141 | catgtttgaa caatatttaa tgaataagta ataataataa taatacatta atacttcttt |
| 28201 | cctttaacca atcaaaacat ctgaatgatt tagtagataa tttcactttt taaatcaagc |
| 28261 | tttgtgtttt atacaaatca taaatttctc aacaagaagt ccacttgata tggaagcttt |
| 28321 | ttttccatgt tctttgtcaa agaaacttta gggagtcatt ttttttagct tttaaaatca |
| 28381 | gagataactt tactctgtca tactgtatag aaaaaggtga tcattcaact atatttttta |
| 28441 | accccaatct cccaaaacac tggtttgatt gaaggaaata tttaatgaga atacactaac |
| 28501 | catcagagat tataagctta tgaatttcaa aaagttcata atcacaaata ttacatatat |
| 28561 | atttaaaatg aaagtgacaa aagaaaataa tatgtagagt aacattctgt gggagccagc |
| 28621 | actattacag actctagaac ttgtcctgat cccatgtttg tatgtaggta gttggagagc |
| 28681 | tctgggttat atttaagtgt aatatatctc tattttttat gttattgagg gaaataatat |
| 28741 | tttaaacaca ctaagtataa tcttccataa ttttataatt tcaaaatcaa aaatttagaa |
| 28801 | atattcaact tctgctttat tcttccaact cttttaatta gagtttcaga aaccaaggac |
| 28861 | ctactatgtg tgaatgtatt acacacctgt gtattacata tgaaaaaagt ttatataaac |
| 28921 | attgagaggc ttttcttagt aataagatac atctttgggg ccatttttta tgaattacat |
| 28981 | ttttgtgtct actttctttt tgaaatcYta agtgtattac taagtaccaa gtaaaggaaa |
| 29041 | gagtggatgc tataaatata tgatagaatc agctcactca tagaaagggt cctgtgccct |
| 29101 | aaaagaataa atcttagaac gttgaatcat ttttgtaact caattaattg agggggcatt |
| 29161 | tcaggtaaac aaatgttacc ttctgtgatg caattcaatc tgttttaaat tattctttga |
| 29221 | ccatttcact ttctttggct gttagatggt tcaacatcat tgtgttttat tactccattg |
| 29281 | tggctaacct taattaaatc ggtagtgtct ctttgactgc acatctgtct tcaattagtt |
| 29341 | tgctctgttg gcttttcttc cctctaccac acagtactta aatatgcaaa gctatcactg |
| 29401 | tacttaccac atgatggcag cactttgctc tgagagcacg gattaaattg tagacttcac |
| 29461 | ttttactact tttatatcac atccagttat ctgaaattta aaaggcaagg tctctgccta |
| 29521 | tcctactaaa ttccaaaaaa ttagtgtttg gaatgtaaat aggttgctgt ctatgatgtt |
| 29581 | ccattattgg ttttatatct ttggtccagc acagagctat ttccaaatat tctgaggagt |
| 29641 | aatttattat gtaggtagct tgtactttcc caaattactt ggttaataca gtagtaaata |
| 29701 | tcttcctaga tacagtctct aaaattttaa gattaaccga aataaaaagc aatcaataat |
| 29761 | tttgtttatt ccaatactta atgatggaac ggaaataact acctagcagt tttagaaatc |
| 29821 | aagtatttgg gaatttttac cccctgtagc tagctttaat ttagctagta tttacttcag |
| 29881 | gtacctttct cctctctttc tttgtttctt tctctgtcca tccctctttc ctttccttct |
| 29941 | tccctcctcc tgtctctgtc atctacctgt tgttattgct gttactgttt ttgttttttg |
| 30001 | atatttttac taaagggatt ttgcagtttt atctcatgtt ttaattttct tttgaacgtg |
| 30061 | gacctgaaaa tttcaggttt taagtgtcat tgtgccagca ttatttggaa tgttttccct |
| 30121 | tgaattatgt acaatcacac ttcatgtatg ttttctatta cagttcaggg gttctagttt |
| 30181 | aagaagaggt actgcatctt aaaagtaatt tagaagttta atttttttcc aaatctactt |
| 30241 | cagtataatt attatctatt tgggaaaatc taaagcaaac tggacactag aaaaatgggc |
| 30301 | tagcttcaat agagctattt gtagttttca cacaccacca aatcacaagt tttcacaaat |
| 30361 | acaggtgttg aaactctgat atctctatca gtcatttgta agtcgttagg catggaagtt |
| 30421 | gaaggaagga tgtcattgac aatttctttt ttaaatgaca aattattaga tgactattag |
| 30481 | tcattacaga tatattttat atatatatgt aaacaatata tctattataa aggtatgaca |
| 30541 | agtcatgaga aatggaatgt tgcaggcttt tttcttataa aaataaaaat atttccctta |
| 30601 | agatatgtca tattcacata gtagctcctt ttagagaaga agatgaagac gggttgcctc |
| 30661 | catttgctgt gttccagaaa gatggtaaat ctcgtatatt cacagttttc tctctccatg |
| 30721 | cacattggcc ttatgactga attcttaatg acatcttaat gaacaattta gtttgtttat |
| 30781 | aatgattcat tatctggctt tatagataat aagatcaatg atcacttcag gaaacagaag |
| 30841 | taatcgtata aaacaatgta acaaatgaaa atgaagttag cttaactaat agcacggtat |
| 30901 | agaggaattt tttcttacat gaaaaaatac atgttggctc cgtatactat aatccttgct |
| 30961 | tttaaaagga aatcataatg ttgcctttta agtaggtctt cctttttttg aggggttttt |
| 31021 | agttcagaag tgattttgga agaaaccact taaaatatag tttaagtgag aattaaaatt |
| 31081 | gtatagtaaa taatgaaaga aagtttgact aagacattat catagatgaa aattaagaaa |
| 31141 | gtaaattcat tttgtcttta ataaattcag gccgtaattc cagctatgtt cgaataaata |
| 31201 | gaatgaaaaa agaatgtatc cagtgctgag ttccggtttg gtgctataaa atgcaaatac |
| 31261 | aataatgatc tttatcttta aaagattaat agcatggggg atcatatgtg aaagaaaaaa |
| 31321 | cattcaattt tgagctacat caaattttca tgggcgattt ttaatttttc aatcttcctt |
| 31381 | ttattctctt ttgaagcaga agcattttct ttcttgctgt ttgcttgcac tacacttgca |
| 31441 | ttaggctgtc agataggtat ttccaactga caattggaga aaaaaaaaac tcaagcagga |
| 31501 | ataaagacta ctgtaaaaca tgcaggtttg tttgagtaaa aagtcattct aaaaatagtc |
| 31561 | attttgaaaa aagttcttac ataatcctga aactgtgaga acttacatca cgactgctta |
| 31621 | gataacaaga atgctgtcct taaatggaat ccgaacattt tgaaaattca ttaaattttg |
| 31681 | cccaaatgaa aaaggttgag cacatttcca gtttgttttc atattattcc aatttggtca |
| 31741 | atggccacga agtaaagagg aagtttggac agttatttga taaatgcacg tctggtctca |
| 31801 | tgagacctaa cctctctagc taatgtcatt aatctttctc caaacagatt atattaatga |
| 31861 | tctattaatc tataacaatc atttcatcaa tctttctcca aatagatcct taaaaaatac |
| 31921 | tttctgtcta tatatagatg gttatattgc agtggattgt acaggttatg atagaaccag |
| 31981 | aggcagaaca aataaattat aattaattat attttccaga tatttttaaa catgatgatg |
| 32041 | tttggttgca cctaaatgta aatcagtgac caattgcatt tttctttttc ctctcagttg |
| 32101 | aagatagaca aaactcactt ttctgactct tcataatttt ttccagatac ctttaaattg |
| 32161 | attattcaat aaatattact agcatgagag attcatgaag acagtaatat ttgtctattt |
| 32221 | tgtttgctgt agtatcacca acaccttgaa gaggttctgt gacattattt gcactaaaaa |
| 32281 | taatatcagc aaatgtatgt aaaaatgatg acctaataaa agtaattcag atcatatttt |
| 32341 | tcataaatga taaacacgtt tgcaaatatc cttttggcat taaatatttt gaaactcatc |
| 32401 | aaattatata cttgaaattg aggatcaagt atttcgtaag aagctttcca tttttcctca |
| 32461 | aatttgaatg aaagtgataa tcttcagttt ttctattgtt atgccaatac aattaatttc |
| 32521 | cactatatgg gatcagtaat gtagataatg gaagatgaaa ttaattgata atttataaac |
| 32581 | aactgttttt ctataaatgt tactttcaag ataagaacaa aaaattttat tctaccttta |
| 32641 | taacacttct gtcactctac ttcagcttgg aagcagcttg attaaagctg ataggcaact |
| 32701 | tatgaaattt caaaaggaaa aagtaacaat tacggatgtg taaatataat ttaatctata |
| 32761 | tcattcccaa taaaataaaa aattgctata taaattgaaa aaatgtcatt tgtcaatctt |
| 32821 | aataattata aattaacatc aaatgagaaa ggaaagaagg ggttctgaaa tgcaatagaa |
| 32881 | gagatatata agatacagaa aatttggaga caaacatgat aaacatgttt tggaagaagt |
| 32941 | ccttggttat tcagtctttg actattgtaa aaattatgat tgtgcataag tctaagttgg |
| 33001 | tgacttttat atatttttaa gcttaaatat atttaaacac ttcaacataa tttaatttat |
| 33061 | aataatttat aaattataat ttaatatact taaatgttta agctggacaa caagcttaaa |
| 33121 | tattttagtc ttatcttaga acttatttaa taatccaaag aactttatgt tatgtaggtt |
| 33181 | atatctactg atatttatca tattcaaaat aaataaaaca ttaaaacaaa aaatcaacaa |
| 33241 | gcacatattc cattagtcat caaagcaatg ataacatgac accttatgta gctgctggaa |
| 33301 | aactccactg tacactcgag aaaaaaatga gagcaaaaaa gcaaataaca tgttaatctt |
| 33361 | ctYaagaata ttttgacctt gcagatccct gaaagggttt ctgggacatc ccccgcccct |
| 33421 | accaggaatc agtgagaatt actggtcaag acccacagtc atggaaatga agtactgtga |
| 33481 | aaaatgagaa gaaactgatt agcatcagtt tctttgacag tcaccttaac aattacatat |
| 33541 | gtcaagatca ttttatcatg ctatttattt ttaattttgt gttttgtact gaaaaaaata |
| 33601 | tcaagttttc agctttcttg aaRagattca ttattgatat tagcattcat tttaattgtc |
| 33661 | tctaagctNc tNgtttcact cagaacttca caactctaga aatatatggc atgaattaaa |
| 33721 | aatatatttt atcataaaat cttgtctatt tttggttaaa aacgaacaaa acaagaaaat |
| 33781 | aactatgtta ttgtgcacag aaacccggag taaataatga taattggcag gcataaaata |
| 33841 | atgcagattc ttccataaag aggttttgtg ttcataaagt atcttttgta cattaataat |
| 33901 | agttggatgg aagcaaaatg gtgattcaat agttattgac tttattaaat gtcattggca |
| 33961 | atgaggatgc tttaaaacaa aaactacaYa atagacctat atagaaagac atttgaatta |
| 34021 | ggtagttgta aaaatatgtt ttctatattt ctctatattc caagttcttt ttataaaaag |
| 34081 | aatcagataa aaatacagtg tgatctatag aacttgaaat acatggtttt caatttgaaa |
| 34141 | tgttttaatg aggaaaagtg aattttgcta tgttcttttt aagaacaata tggtagaaat |
| 34201 | tatatttctc tacctttctt atacatttgg aaatagaaac ctgtgatcct gacttttacc |
| 34261 | aaacatttct tctgaaaaat ttttctgcct tgtttgagtt gtggtattag gttggtgcaa |
| 34321 | aagtgattgc cggttttgcc attactttca atggtaaaaa ccaccattac tttttgcacc |
| 34381 | aacctaatat tattttgatt aatctgttca aattataatt aaattaacga attatacttt |
| 34441 | tcagtatcta aaacacatta aaatataaaa tgatggttag atctacgtca ttcctattca |
| 34501 | cacatctacc aaactcaaca ccccgatcaa aattttatat ttcggatata atttggtttc |
| 34561 | atttgaaaat tcatgtgttt ttggcttttt agtgaYtcat tatatattta catgtacagc |
| 34621 | acagtaataa aaatggaaaa gaatattttc atggtcttgt tcagtataca tgaaaattcc |
| 34681 | cttcatctta ccccataccc acgtttcttc ccccagatca tgttgcacaa tgRagaatgg |
| 34741 | gtgaaatatt atggtttaca tgatgtatgt aatacatgtg gatgaaaagt ggaaaaataa |
| 34801 | ctaagcattt catggatagt tacatatctg gatttgggga ctattattaa atcaacaatt |
| 34861 | agtgtctttg gcattcctct accactcctt gggcagcata tccctggttc catgatcctc |
| 34921 | ctatccaagt gagaaagttg cctgccccat gctcttattg taccctgact gccccccact |
| 34981 | gtgtctccat tggcattctc attgccctcc cccatcagca tttacttgcc tgtcttactt |
| 35041 | ccacaggtcc tgctccctgg tggtaatttg atgaactaac tttttgaaaa taattgcatt |
| 35101 | acaaaaacta ggctttatgg gtattatttt taaagacatg tcataaataa tatttaagaa |
| 35161 | accttggtat gttaaaattt aggcctttct cttcttctta gatcatatat taatagtctc |
| 35221 | atcctcatta tttaggccca tagctagacc atatgactaa ggcataacaa ttttgaagcc |
| 35281 | aagtttggtg tttagatggg ccagttggtc ttacttttca cgtcagatga aatgaacata |
| 35341 | taaatcccca ttgtggacca gaccaaagta tgcagatggc tcggtgcaga tccatggcta |
| 35401 | ctactggaaa agaacagaag tacccattga tggtaggtca gtgaattcat cttcaggaat |
| 35461 | gaagatccat aaacaagaga tagtgtgata tggtcagagt tctacattta ctgtctgatt |
| 35521 | ctgtatgtat agcagaggta tttccagcaa taaatttaaa tgactataga aatatttgaa |
| 35581 | aatgcatagt caatcattcc aagaaaatgt agaatctctc aaaaccaaaa cgttagaaaa |
| 35641 | gcattttgat tttcacagac aacaaaataa atccttatca ccagagaaat aggtatagaa |
| 35701 | ctctgaaagc tgaaattgtc caaattgatt ctctcagatt ttgttggatg tttgctatgc |
| 35761 | gcatgtcatt acagtttgaa ttttcttttt attttaaact gctcaaggtt taaaaaattt |
| 35821 | ttttgcacaa acacatggaa atgaggaatg taaaatttgt ttttattaat cttgtacata |
| 35881 | caatgttaaa gaagacttca cctccaaatg aaattgattt ctcttatgct ttttccttta |
| 35941 | tttttcttat gggcctgacc tttacagaaa cagggaaggc taaattcctt ctgaccagaa |
| 36001 | gtgatcatct gcttctggtc aaaagaaaat gggatgctga ttagctacta tcatctgtac |
| 36061 | ccagcaattc cctccactcc cagtcccacc cactcccaaa cccatccacc cactaccact |
| 36121 | tgcccaaggc agcagctccc acagagtgat ggcttagtaa gagttcacca aggcaacaca |
| 36181 | tggatatgaa actcttcttt gacaggtgcc gcaaatgagc tttattgaaa gcggagtttc |
| 36241 | cagtagtggt ctgtggaagt agcatcaggc ttttctccta caattcagcc acatcaggga |
| 36301 | actcgaaatc atatttcctt gcatgaccta tttaataatt tattgtctca cctaatctct |
| 36361 | ccaacctgtc ccagtggctc ttgttttatt gaccagggta gagggatatt tctcctcatc |
| 36421 | ctgtattgcc tatttctaaa tttaaaactc atacctaaaa tcttagtatc aaacaaaaat |
| 36481 | agctcaggtt tttcaMactt aaatttagta tattggattt caactaaata aatcttcaag |
| 36541 | gttttaggaa tgcttcaact tgttttggtc ttgatatagt tttcaaacaa accaacaaat |
| 36601 | aaaaaatctt ttgaaatggc aaggtttgtg ttcaaccaaa ccaactagca tggggaaaac |
| 36661 | aacaacaaNa accgaaaaca tttccagagg tttttaacta cggagagttg tttaggcctc |
| 36721 | aggatgtttt tgcacaatct gtctaatctt tcccgtcagg tgttttagca tacaactaaa |
| 36781 | ccccagaggg aaagaaaaaa ttccttttga ctcactccat aattcttgtt tgacccatat |
| 36841 | tttatgctag ttcaagcaga aatctgctga gaagcttaca aaaaatcttt gaaaagctct |
| 36901 | ttagggacaa ttttgtcact cttgatagaa atgcctacag atggacccgc agccttttct |
| 36961 | aatggagcta caaacattgc ctaatgcctc actgggcttg cacagtggga aggcttcctt |
| 37021 | ttgttgggtt aaatcaacaa cctttagaaa ggaggaccat gtaggcagag agtataaatg |
| 37081 | gaattccgcc atcatttcca gctacctcta atcgtttgta agaacttaag agataagtca |
| 37141 | taaataagta catgactgtt cttattaaaa ttaggagagt ttgattttaa aggagacaat |
| 37201 | gtattcaaaa attataactc aaaaagtcac aagacaatgt tatatttgtc ggttctgaaa |
| 37261 | atttctagct gtgtgttctt atatcagcca gcctacccct ctgactctga ttgctcacgc |
| 37321 | ataaaacagg gataagaata ttgaaataac Yacctcatcc acttaatgtg ataaaatgag |
| 37381 | aaatggatga caaaacactc tgtacaacat gaagaactat tcaaatgcag agtattattt |
| 37441 | tcagaatgcg gatgggatta tacgaactcc attgtgaggt ttgaaatggg acacttttat |
| 37501 | ctcaacactt atgattatta ctaatacttc tgcagttagt gaacaggaac ttgcaggttc |
| 37561 | tttttatatg tttgtatctc tgcctagttt ttctctctgc actttaaaca aattatattg |
| 37621 | cattttatat aaatttaaca ctatagtatg tttgcYtgaa gtgcctggaa atatacatgg |
| 37681 | atcaattata ttgttacact tataatagca gaaaaattat actttagaaa acaaggcttt |
| 37741 | ttaatttatc gtctattaga acaaaattaa acatgaaagc atgctgtgtt atctagtttt |
| 37801 | cctttcaatt atgattttaa tgaagagtga cttctaagtc aatcacaaga ctaaatgttt |
| 37861 | gagaatatct attagaacac tattcaaaaa aagggattaa aaacacttta atataaattt |
| 37921 | taaggactat gtctaggaag aaaatcatat aaaatgtcat atagaaacac ttaagtcaca |
| 37981 | ataattaaaa ataaggtgtg aaataaggga agtgtttgat tactgtgaaa tttataattg |
| 38041 | aaagaatatt taaaNaacgt gaataacatg aagaaatcaa ctgaacttat ttgatggaag |
| 38101 | aaaaggtgag aggtggaatt ttaaaaatga aagttttagt aaggctaaca agaaaaaaga |
| 38161 | aattgtgctt tgatgataat ctataaactg ggctgagtaa cacatattca gaattatcct |
| 38221 | aatgtttaag ctgattgttt ccttaattac ctaatttagg aaacacctca gtggtttacc |
| 38281 | tctgtaatgc gacatatatc ttacattgcc accacatata taattaatag ggagaaggac |
| 38341 | atatgaaatg tttttaattt tatgagaata aaaagaacgc aataggaaag tataaggctt |
| 38401 | gattttaatt ttgtgccaaa tatagactca taaaatcaaa tattttaggg ctgagaggta |
| 38461 | cttaaggggg caaatagtac tatcctattt taaaaaaaca ttattttaat atgtgctttt |
| 38521 | gaattaaatt tgaagataat tgtataatca ccctagcttc attccttgtc actggcagtg |
| 38581 | gcactctttc ttgggggaaa attctagcaa tgttggggtc aggttacaga tggatgtaaa |
| 38641 | tttgctttcc tttctgaaca gagctatgag ttgaaggttg gccacttctg caaagcaaga |
| 38701 | gctcatggaa tttaccttcc tctatccagg aacctgagaa gattgcaaag tagatcagac |
| 38761 | gtgggagaga gaaatagcag aagtgttgcc tagggcagcc caatcccatt atttatttac |
| 38821 | attttaatgc aatctctgat gctttcattt ggtgacaatt ttccatctat cttatagtgt |
| 38881 | ttccaagatg gaactttgtc ctgctctcaa atgtccaatc attacagctg tcactagcaa |
| 38941 | tgaggcagta attaaaatct attgcaacga aatcaattga gaaaacaaaa ctgacacatt |
| 39001 | cattttaagc tgcatcatat aacaatggct cagaacccag tagtttatga atgaaataaa |
| 39061 | gatactttat ttattacaca ggcacttgga gattcacatg cccaacacat ctgatacact |
| 39121 | taacaactat ggaaacacga gtggaaaatg tctgtttgta ggatcaagag agaactttca |
| 39181 | ccatgtaaag gtaaatgagt gcattaacaa tttttccttt actttcctct aagaattttc |
| 39241 | aaaatccact aaatttttaa aagttacact tgaaataata ggcaaMaaac actgatccac |
| 39301 | attccaactg gtaggctaac ttgaactgtg agcaatttaa acttttatca caatgaaaca |
| 39361 | tatttatagt catgcttgaa cagattaggt tagtagcatg cattccattc tgcaatatga |
| 39421 | atattgttaa tctatttccc aacaaaagtc aaaagatgcc cagtgcatag aaaaatacaa |
| 39481 | atcactgtag tcaatatatt acgggactga tcttgggact tacatggtga tgatgaagtc |
| 39541 | ctgaaatgcc atgcccacaa aagcacatta aaaagtggat aaacatattt tatcagacaa |
| 39601 | tctctaaaaa ttaaRgatga gagctttaaa tttggggatg ttcttgctgt gtgctttgaa |
| 39661 | cagggccttt ctgctcacct ccttctggct tgactggtcc atcagacatc accttcctga |
| 39721 | gggctagctg gctttggtct tggttgccat tggctggtgg aggcagatta tcagactgag |
| 39781 | ttctttgaat ggggaggact cctgctatgc tgtgtctgtg ctgcttcctg gcatgattag |
| 39841 | actgaccgct tttatgtgct gctgtggcag tgtgggtgaa atggaaaccc agagtatgta |
| 39901 | tcctcagatg gaaccagctt aaatgttatt catgcaaaca gaagtgctct tttgctctat |
| 39961 | ttaaataggt tgtgttttca aatgccaaaa tgacaccaca tgcacagcac taaaggggtt |
| 40021 | taaaactggc tgtttctttg aaaatgatga ctagggagat ggagagaaca atgatctatc |
| 40081 | tccattgcct ggctaatgcc ttttagaagt aacagggtac ctacatttgt gaaatatttg |
| 40141 | cacatattaa gtttttatat aattatatca atcgactaaa ttttcacaaa tgaaatgaaa |
| 40201 | atacagcagt taagataggt caattgttat actttgattg acttatagct accagacata |
| 40261 | gtatatcttg atttccaatg gttagaaaaa atatctgtaa aatctgtcat cacatacaaa |
| 40321 | gtgtgatcca acagaatgga cccaattgtg ctcctcagtg aggttgcatt gccttatcac |
| 40381 | atatgcttat acatatatta atttaatgta gttttttgag aatgtaagaa ataagatttt |
| 40441 | cttcacagat ttttcagaag actacaattt tttgcacatg gaaagctacc atatttcaga |
| 40501 | agcaaaattt gttagtgttt taaagcactg tcttgaaatg gcttttcctc caaggttcta |
| 40561 | gaagttgact gttggttgca ttatcttctg cctagacttt ttgagtgttt tcagatacaa |
| 40621 | gaatctaatt gcaaatctgc tctaaatact tatatgtgtg cacttttttt tttttggtat |
| 40681 | aagtcctgga aaactggcag ccacttgtgc ttataatttc tgtattctaa aagatattta |
| 40741 | agcgcaaaca aaaaggacac acaacattgc tttgcttcca aacatcacag acactgtatc |
| 40801 | aaatgctaKg agccaggttg gatggatggt ggaaacaaag gagaaacagc cagttttatt |
| 40861 | tcagtctatg taaataaaaa aaatctaaat aagaaagagc atattaatgt atttatatcg |
| 40921 | ttcttacaca gacaaaggga cagagaacag ggtgagcaga ggatgttgaa aactgaaagc |
| 40981 | aaacaggaaa ggacagcagg aaagaaaggc aacagatgaa cattaagctg ccatgctgag |
| 41041 | gaatttattt gcgtctttta cttggttgaa tgcggagttg ttgcacggca gcttcggcag |
| 41101 | cagctatggc agcccctgca tcttccaggt gggtctgagt gacgctggtt ttgctttgac |
| 41161 | tgcgagatgg tccatgagaa cggagatggc cttctgatga tgattttgag ctaccaggac |
| 41221 | tactatggga tttctcaatg gtgggaacct gcatgtctgg tcacaaaagg gtaaaacatg |
| 41281 | agttaaagtt gttccagcat tcctcctatc aagcttggtt tacacataat ttgaaactgc |
| 41341 | ctaaaattta tgttaattct gagtcttata tgattcattt atgcaactgg tcatcataca |
| 41401 | gaatgatgat taaacatctg ggaaacaatt tgggggagga aaggcaaaag gatgatagca |
| 41461 | atgagaagag agaactttga aaaaagaaat acattctgac tagcttctag taataaatat |
| 41521 | gttaactttt agtttcacca agcacctctg ctaattccag cagaggtcag gtaggagaca |
| 41581 | atacacagga ggagttttaa gcaaaaacag gatgaaggat gtgaatcact aggacatatt |
| 41641 | aagtgtggag attgtagaat catgcctcaa accattgtag agaatgtatg gatcacgtat |
| 41701 | ttcctaaggc agtcatagaa agagatcttt atactaatga tcatgcagta tgttaagcaa |
| 41761 | gataagagtt gagatggcta agtggaaccc ataaatgatc tataaaactc attcataaat |
| 41821 | aatctacaaa actcgtagtc agcagtttat gatatcaact cagtggccta ctggacatgg |
| 41881 | actcactttc tcatctccta gaacacttgc atttatgatt cagagaatct catgtacaaa |
| 41941 | gaaccacttt acaatttgct ggactttcct agaaaaggtg acaaggaagt aatactcaag |
| 42001 | tccaatataa aaattatatt taatacttac aaatagttca gtatatctag gtaaggtacc |
| 42061 | acWtgtttta tcccacagag gcagtagctc agtttatgat gaggattaat gttgtgttgt |
| 42121 | aggacagttg gttatgaacc tctcaaagga aaagctcatt tgaatgagag tgctgtacat |
| 42181 | atttgcatat tgccaaaccc aatgtatggc actgttattt aaaaaataat aatagttatt |
| 42241 | aaaaaattag cttgagtcca acatttagga aagacttaag taaatttcag cataccaatt |
| 42301 | gttttgatga caggggaaaa tattctttgt atgataaata aaaagtagca tgcaaaatta |
| 42361 | tatttattgg gtaagacaat atttatggac aaccagagat tcaaacaatt agaaggatta |
| 42421 | taacttaatg ataatgatga ttatgtctag tcaatgaact cgtagggatt tttgtattat |
| 42481 | ctttatgaaa tacccaaatg cattttctga aatcatgtta tcatggattt tgaggtggaa |
| 42541 | tagaccaaat agctcatttt acatgttgaa attggacagt tagagaagtg aagattacaa |
| 42601 | agtactacac ctaagtatgg gtgatgatct gagaataaag cccatttctc ctgattccac |
| 42661 | tctttgcatg acattatatt aagtattttc cagtcgtgtc taatctatta atataggtta |
| 42721 | aatgtacaaa cattcacact aagaagatac agactgaaca aatagacaca aaactttttc |
| 42781 | tacttaaaaa aatatttcct accctttgat gggtcaggga agataccatg gcttctgctt |
| 42841 | ttgataacag atggctttgg ggactgctgg ctgctctgac tggaatgaga cttgccatga |
| 42901 | tcaatgcttt cagtctgttc tttgagagga taccaccttg gagtgttatc gaggtgagMt |
| 42961 | gtgctagata aatcaatcaa tacctgaaaa aaagtgtaac aaataaatga aatttaggga |
| 43021 | ctaaagtata attgattcat tactttttct atgttctaaa atttattcaa ggatataaac |
| 43081 | caaggaatac agtataactt ttgaggatga tgattctttc ttcctcagtt ttcttcacca |
| 43141 | atgatactgg aaacaatgtc ttttacatac tagatgctta ataaatgttt gcttgaactg |
| 43201 | aactgtataa aatttttgac tattaactaa attatttgga tatgatgtta aaagataggg |
| 43261 | tcacaagtca atctgttcat aaaacaaaac ttctttttgt tgtaagaact tattttaatc |
| 43321 | ttacacggtg aaatcccgtc tctgctaaaa atacaaaaaa aaaaaattag ctgggcgtgg |
| 43381 | tggcgggcac ctgtagtccc agctgtgtgg gaggctgagg caggagaatg gcatgaaccc |
| 43441 | aggaggcaga gcttgcagtg agctgagatc acaccactgc actccagcct gggcgacaga |
| 43501 | gcaagattct gtctcaaaaa acaaacaaat aaacaaacaa acaaaaactc ataacttatt |
| 43561 | ttaatcttaa actggactag caaccttgaa aaatgtttga ttttagtcac aaaagggaca |
| 43621 | aaatggaaaa gaatgagtaa gttgcaatta caaaSttatt tattctattt taaaacatac |
| 43681 | tttagttcac atttgaaata gggatatgac taccacattt tgtcatggtt taattggcaa |
| 43741 | cttttttttt cttagtacat tggtgcattt tacagttgct gacatcttag atatgatgag |
| 43801 | ctaaaaagaa atgaaaacca gaccaatgga tatatgcttt aggctcatgt aaacataaga |
| 43861 | ccataaatta gaaggacttc agtaagtcac ttttttgaaa tgaaacataa agtcaggcat |
| 43921 | aaYactaatc tagagtactc ctgcctagga tcaagattag tccaagtggc tagagctgga |
| 43981 | tggaactgga aggtaagggt caaatgctcc agttttggtg aaaagagatg tgcaaagtct |
| 44041 | taagaaccaa acaagtcctc attatcacct tgattacaca aaaggatgtc gtattttgta |
| 44101 | ggttttggat tttttggaga ttatttttaa cattaattca taacttctgc cctaagcaga |
| 44161 | acactaaaca atgtgtatgt gtgtatatat attatatata ttatacataa tatgtatagt |
| 44221 | atatatacat tgtatatata acgtgtatat atacattata tatacactgt agacatacaa |
| 44281 | ttgtatacac tgtatagtat atattataca ttgtatatag acatatatac attgtatata |
| 44341 | cacatgatat atacaatata taaaatatat gtatatacaa tgtataatat acgtatatat |
| 44401 | aacatataat gtaaatgtgt atatatatac acaatgtgta catatctaca ttatacatat |
| 44461 | aatattccta tatataaaca atatgtgtat atattatgtt catatatata aacaatgtat |
| 44521 | atatataata gattaaaaat cacaagatct gttcaYgcta ccaatttcat gattatttgt |
| 44581 | ttatttttaa atttttacaa gtacatagta agtgtatata tttatggggt atatgagata |
| 44641 | ttctgataca ggcatacaat taggttattt tgattccttg atgttaatgt aaaaattgtt |
| 44701 | tttattgttt tctcctactt gtagttttgc ttcttaaaat gttttcagtg atttttctca |
| 44761 | gaatacttaa ttataaataa caatgcaatc aatatgtcac atcaaatcaa ttaaagaacc |
| 44821 | tgtaaatgca tttttaaaag tccaggccat tttataatat aataaatttg ttacttcaaa |
| 44881 | tctcatttgg actcacagta tgattacata tcttcctatt gaaagaaaat ttcatcttag |
| 44941 | aaattaatta taaagtacat caagtgatag agtgaaaacc atttcttgaa ttggtattaa |
| 45001 | tctactcaaa tagtaaatca ggaaataaat tctatctgcc agaagatttt tttcagaaac |
| 45061 | ggctatctta taaacttcag aaacatgctt tgcatcgtgc tttaattggt ggtttaccat |
| 45121 | agcagcaaat aagggaaagg aagtcRgagg cttacctccc caaggaagtc gttggatgaa |
| 45181 | aatctatcat aatcccaaac tgtcacctcc agtgttttct tcttgagcta tatagttcaa |
| 45241 | atagaaatct aattaaacct atctttccat catacatttt cattacacac atacagtaga |
| 45301 | catatttatt tttttcctta aactccttcc atttatgtga aaatacttaa aattttaccg |
| 45361 | aaaactatgt agtttaaata ttaaatatta tgtaaaattt gaatgatttt gcactctaaa |
| 45421 | ctagtgtttg atcatgggga acattattat tttgtgaata aaatgataat tcttacttat |
| 45481 | ttattgtatc cccacctcat tttcaaaaat agaaatcaga atttataaat actctaaata |
| 45541 | taagaaattc atagattgca aaaatgtcta gtattatcta gatgaagtat tggattaaat |
| 45601 | gtttcagcaa tttccaaaaa acttagtagc attgccttat ttctgcaggt gagtttattt |
| 45661 | taaattcaca aacccagatt ataatttata tccagcttca tctttgtaat gaaattaatc |
| 45721 | tactagaaga caaattaaga atctcatttt gaaagacagt tcacttgtaa attggtttat |
| 45781 | gtgggaactg tctttatagt tttctctctg aattcaacct tatacatttc agttattgtc |
| 45841 | tgttaagcaa agatataaaa atgcagattt tcagaaatat aataatgtaa tttttgccta |
| 45901 | cagttactta tctStggtga taatccttta tactagtcat cattttgcct tcttctgtag |
| 45961 | agattagcac cacaatgcca gattcttagg aggcctcaat ttagcaaaga gatgagatcc |
| 46021 | taaaatattg tctagtggag taatacagtt ctatgcactg aatcttattt tttttgtgta |
| 46081 | ttgtgtatga aagtcaaaat atagccagtt ggaaaatgtg tttctctctc aaatatgata |
| 46141 | aatgtggctt agtctattaa aaattttaaa ttctgaaata acaaatggaa aatattccac |
| 46201 | tagttttctt attttatttg tttatgtttt ggctgctttg tcaaaatgtg cacatttact |
| 46261 | ttcaccctgt gcttaaatgt cactcatttt tttccattgg ctacatttct tacatgtatt |
| 46321 | ttactcaact ttttttgttt gattggcaaa atgtatacag atcccaattt ttgaataatt |
| 46381 | gtgttatcac tgtacttata ttagcctaat tctgtcacct agaaataatc ttgacatacc |
| 46441 | tgttccatgg aaatactttt ataaattact gtttgattcc actcaggatt aagacttttc |
| 46501 | tggacatgtt tagtccttct cttgtactca gcactgaatt gggagaaaag aaagagttat |
| 46561 | cttgattatt caccttagtt tatgacttca cagttaaact aaaggacatg cagactattc |
| 46621 | aagatatttt atttcaaatc actatataat tatctcatca tcctcatgtc ttaaacctta |
| 46681 | gtttaagttg aatgcacagt gacaatttaa aaatatgttg gatgcttgta gttttctttg |
| 46741 | ccctWatgat ttttttttaa agaaatgctt gtaggtgttc taaatatact ataatttcag |
| 46801 | aaatcttgct cattcctgaa tgtaggagat aattacatga actaaatagt aacatcaatg |
| 46861 | ttactgcccc ataaacaata atttggtgtt taaccttact atagttttgt acatacRgaa |
| 46921 | aagcaaaact aaaacattgt aatgagtgta ctgagaaaaa gatgaagaat ggctgttgct |
| 46981 | gtggctcaca ttccataaac acttgttact gatatctaaa gtatattcca acactcttgg |
| 47041 | taaagtatat ttaatgcagt tataaagcaa ttttaattaa atataaattt ttggtttatg |
| 47101 | tcaatatgYt attttacaat tgatgaacca aataaaaatc tttcctcaat tattagaact |
| 47161 | tgggacatct gcattaatcc ttgactttcc atttcctctg agcaccaatt agaagcttat |
| 47221 | atgctacaca tttaaaaaca atttattttc aaagcctatc ctaatgaagt ctggcagaac |
| 47281 | atctatagct ggcaccaacc tgctgatggg gttgaaatta gattactgtt acttgctatt |
| 47341 | accaaatggt gcccacacag acaaggcctt tctctaactc tcactgcttt tttctgaatt |
| 47401 | ttctgcccaa gcttatgtct cacgttctaa ataatacttc atctgtggtg ggcactagag |
| 47461 | tcctgtaaac caacctctct gtctgggggt cttgacatgt gtctggattt cacaagttgc |
| 47521 | ctttccctaa aagggggctg gaccctgact cttgtaatct gtagttctat tccaaactat |
| 47581 | agccagacta actcttgcta cattcctggt ctcaagtatt gccatatcct ggatgcctag |
| 47641 | aaattttccc cagccactaa tttacctata cattgtagag aaagctattg aggatcaaac |
| 47701 | tccagctcac cactcacttt taaagatggg cctgcaggca agtactttcc ctgtatctct |
| 47761 | gattttattt tcttaccagt cagatacaag ggaaataacg ctcaacattg actgcataat |
| 47821 | gggatcacct gagttctggt ggtgggaggt gggtcagtca accctccacc cacttccctc |
| 47881 | ttgcccctgc ttactccccg aaggaaagtc tgacttgatt agtctgggat gaggcttgag |
| 47941 | tattaggatt tttgaaggct cgcaggtgaa tctaatatgt agcgaagatt aagaagcacc |
| 48001 | gagttggtgt ttcataacac ttcacaagtc agccattgtg agctgaatcc aactgaagaa |
| 48061 | ataaagcagc attcctcgca cttcaaccta tttgaatgta cacaattaag ggcagaattt |
| 48121 | gacagctgtc actcaagata aattacaaag aattttaggg aaatatacag acaatgattt |
| 48181 | aaagaaagag gagctctcat agactatgga taggaagttt ctatatatac tttttagata |
| 48241 | aatttgtcaa tatctagtga agtttaagtt tatataccca aacatccaaa tattcaattt |
| 48301 | ctactaattc cacttctgcc atagagaaat tctcctacct gtgaacaaga aatttgttca |
| 48361 | agaacattaa ttgcaacatt atttcaatag taagccaata gaagaacgaa taaatacatt |
| 48421 | gtgatatatt catttcaatg aaatagtaag tggcactgaa aatcaattaa attttaaaag |
| 48481 | cctccatatg tcaagataga tacatcttga aaaaagtttt gatcaaaaac ttaatatggt |
| 48541 | atggtataat ggtataaaca taaaatcatg tgaaataagg ttacacatta tatatacatg |
| 48601 | tatatatgac atacatatat attaatatcg taagaaaact tgtaaattat gaatattaaa |
| 48661 | ttcaatttag tgagtttttt aatggatagg gaagtgattt cagagggctt cagggtatta |
| 48721 | tcttttaaaa aatctaaaac aaatatagca aaatcttaaa atttgacaaa attggatagc |
| 48781 | aagcacacag gtatttgtta gggtattttc tataattttt agtaaacttg aaatatttta |
| 48841 | taatgaaaaa ttcaaaaatt tttagtaaga tatgttcctt taaatttagt tttggattct |
| 48901 | ttttttgatg gtgacttaga actaatgact taatttgatg gtgatagaat tagtgtctac |
| 48961 | aaaaaagtgt ctacatagaa ataatacata taaattaata catagaaaga gttaataact |
| 49021 | ttaatttgat agtgacatag aaagaattag tgacgacttt taagttaata gtaaacttgg |
| 49081 | attgctactt tagaatcatg gctattctca taggtgaata tacataggaa acaaacacat |
| 49141 | ggtagtatga gttttaaagc ctctgttttg gacttactta tcaaggtggt gaggttatgg |
| 49201 | aaaccatatt aagatttata atacactgaa cagtaataaa attatgtttt ttaagttcta |
| 49261 | aaaaatacat aatttgttta acaagatggg aattaatatc aaatgcaaac ccatgtacta |
| 49321 | tcactaaaat tctgggtctg ccaatacata ttaaaatagt ctgaagtagt attaaaatac |
| 49381 | ctttgaaggg atttatcagt cactatcttt tcttaatcca tacatgtgaa taattgttat |
| 49441 | tcaaattttt caaatccaca tttagtcttt ttttcactta tacacctagc aaaaattagt |
| 49501 | gaatgccaac tctgtatcag gaattgtaga tgtctatcaa ctaaaaaacc cttaattgta |
| 49561 | ttgacatatg agtatatatt taattcatgc atgaaaagca tttttaaaag taagcatatt |
| 49621 | aaaatcataa ctaacattta ttaaatgcct cctttttcct ctggtaaagt tctaagtgct |
| 49681 | tttcatgtgt taactcatgt aatactcata ttaaccccat tatacaggta ttacttctta |
| 49741 | ctccatttta taggtgagaa aacttgaagc tcagaaaaat taaggYgtat tgtcaaagtt |
| 49801 | gcatagttat taactggacc aggatccaaa cataggtaat ttgatttctt gcttttaaat |
| 49861 | aaatgttata tgaactcaat tgattaggcc catcataaaa tgtgacatcc atctattttc |
| 49921 | ttaattgcag gcttcactac ctgaagtatc tagtttgtct tctttataat ctaaaattct |
| 49981 | ccatctcaat ttaaatacat aatttttata catataatat aaattatgta taatgtgtgt |
| 50041 | agacacgcat tataaacatc gtgaacaagt taggccttct ttagagttgc ctagggaaat |
| 50101 | tctgaatagt acatccKaag tgagtgttgt ggaacaatta cgcctactta ttgtaaatac |
| 50161 | tgatgtttgg ccagatacat ttagaaataa cctaaaYtga ttggacaaaa gttaaaatgt |
| 50221 | tcattttaat agagtatctt aatcatagta atgagtttga ttctccaatt ctctccagac |
| 50281 | ctaaaacatc ctttagaatt aatgaaagta aaaacagctt gctcctaaac accaatgaaa |
| 50341 | gcctttgcag tattttttgc ccttgcattt cctgacatgc agagctggtc acacaaagcc |
| 50401 | attcttcaat aacagttgtt ttataactac catgctctga tcatatcata cacaatttca |
| 50461 | atgaactgtg ttgccaccta acacaattta aaactctaca caaaatagtc atatcaaaat |
| 50521 | ctgtggagtt gaaggtaggc aggKaaacag ggggattctt aaatgtgagc ataaagtcag |
| 50581 | gctgagaaat aagtggagtt catcctccct ttcctttctt gtctagtaaa cacactatat |
| 50641 | tctcttgtgg gaagattaca ttaatgattt aacattagga caattaggac aattatatct |
| 50701 | aataattaaa gaagtttatt aataagggaa aataaaaagg tcaatacata aagatgggac |
| 50761 | atggtgctta tcaaaaaata tctaaaactt caccctcttg aagttttgcc ttgtttgttg |
| 50821 | gtttgattat ttattttttg agatggagtc ttgctctgtt gctcaggctg gagtgcagtg |
| 50881 | caatctcggc tcactacaag ctctgcctcc tgggttcacg ccattctcct gcctcagcct |
| 50941 | cctgagtagc tgggactaca gacacccacc accatgcccg gctaattttt tttgtatttt |
| 51001 | tagtagagac ggggtttcac catgttagcc cggatggtct tgatctcctg accccatgaa |
| 51061 | ccaccctcct tgggctccca aagtgctggt ggtttatttt tttatttgtt tttattggtc |
| 51121 | attttgtcat gcataggtat atatactttt aaggctgtct gaaaaaattt ttgaaattca |
| 51181 | attattctac tcaagaccag caggaaaaca gtttccatgt acatattttt taatttcaaa |
| 51241 | agaaaaactg agaaaagata atgaaatttg aaggcttctt tggtgtatct actttcataa |
| 51301 | aattRtctaa aacattgaaa gtattcattt ttaatgattt atctggtttt cctttactaa |
| 51361 | acttactaca cacacacaca cacacacaca cacacacaca cacacacaca cgtttttcaa |
| 51421 | aattactcct ctgaggttat ttctcttaat ttagaaaaaa tacaacttgc atcccatttt |
| 51481 | gtaagctctg tttaatagtg cgatggtgtg cttattgtct atcattttat tttcctcttt |
| 51541 | gctctctgat ggcagagact atatggctac attgtcaatt gtaacccctg cacctggcac |
| 51601 | agtgtctgac acatagtctg tgctaagtaa atattcagtg atccttagaa catgcttgta |
| 51661 | ttttgttttc ctttaaaatc tactgaagta aaataaattt tggacaccat attgtaaata |
| 51721 | cacacaccac ctgtacctgc tttccacagg tcccagagga aatagtttga taggatacca |
| 51781 | atgttgttta gagaaaggtc cagccaggaa ttgggcttcc aggtataaag atagctaaaa |
| 51841 | ataattatat tcataacaaa atctcagtaa tgagaaaata ttttcacttc gctcaacagt |
| 51901 | gtgatgagag aaaaccagag cgaggccctt catatatgct catttccatt tcttaattat |
| 51961 | ccaacaggaa gtattgagtt cacgcacgac ccagaccttt tcctagtcat tctctcacct |
| 52021 | tatcttgcct gacattttgc ccctcaaagt ttgagtatat tgatcagagg gtgggaagaa |
| 52081 | aaaaRtaaag gagacaagga aaccaaatca gtgtactttt taactagcta acaggactct |
| 52141 | tattttaaat ttaagaatga aagatgtatt acattttaat attaaagaaa agttgtttaa |
| 52201 | ttttctatat agtattttaa aataatttag tataattcag aagctcaagc attctgtacc |
| 52261 | tttctgttgc agagataacc aaccactggt cgcaaaaaaa tgtatgatac acattgctag |
| 52321 | caataataat aacaactact attgattgaa agcttattat gttccagaca ttatgcttat |
| 52381 | cacttttgat gtatttattt catgtatttc acagaaggcc tgggaaggga gctgttatta |
| 52441 | ttcctatttt gcagaagata aaatagtttt agagaaatct taaagcttct aagtgacagg |
| 52501 | gctaaaattc aaacgcaagt tcttaaggac ctcacttatt ggcttccaca gtctaactta |
| 52561 | caatgtcagt accttgtcaa tataattccc tcttacattg acaattctca aagacatggt |
| 52621 | gaaagtgatt tctagatgaa agatataaga aagtagcttc tagatctttt aaatagttat |
| 52681 | ttaagacatg gtattattta taatttataa aatattc~ct cttttataag ttaaatacaa |
| 52741 | tctctatcat tctgataatt ttccccaagt ttctatatat aaaagatagg gagattctga |
| 52801 | ataatgtgat taataatatc tgttctaaaa ataacactat ttctttgaaa caaaactaat |
| 52861 | tttgcatatc agcctgagat acctggattg tgttctgtga tgagagaacc agccataaca |
| 52921 | aggaatctat cacagcgtca gttacattgt ttgccataat tatgtggctg taaggaaaca |
| 52981 | aaaagtttct gggcaatagc aaggctgtgg taacaaatcc tattgtttga aatatttacg |
| 53041 | tatttatgct tttaaaaatc agaaacagcc tcatttggga aacagttcat tcgtagtaaa |
| 53101 | tatgtgaaat tttaatgtca tagacaaaaa cgaatgctgt accattaata atctgggtgg |
| 53161 | gattaaaatt cccatcaccc tattgctaca gatcatacat gatttgcctt cccaactaaa |
| 53221 | tgacacattt aatttataat gcaatgagag atgtgcaaaa taatgctttt ggctatgtac |
| 53281 | taagaaattg tcttagttga cagtgtaatg atgcaacttg tcatgagctg tattagaaaa |
| 53341 | cgctcctact attgggaaca aaggaagatt aacttattgc taatatacta cagcaagcca |
| 53401 | attatggcat cctacattgc atagcagaaa ttttgtgggg cagaagttac aaaggcatga |
| 53461 | aaatgtttca acattaggtt caagtgaggg accaagttca aacacctatt atcttttttt |
| 53521 | ttNtttgttt gtttgtttgt ttgtttgttt tttgaggctg gagtgcaggg gtgcgatctt |
| 53581 | ggctcactgc aagctccacc tcctaggttc acgctatttt cctgcctcag cctcccaagt |
| 53641 | agctgggact ataggtgtcc gccaccatgc ctagctaagt ttttgtgttt ttagtagaga |
| 53101 | cggggtttca ctgtgttagc taggatggtc tcgatctcct gaccttgtga tctgcccacc |
| 53761 | tcggcctccc aaagtcctgg gattacaggc gtgagccacc gtgcctgatg ggatatacac |
| 53821 | ctgcactgga catccagaag acScattaat tacttttgaa aaatggctac agatattttt |
| 53881 | aatagaagac tcgagaactt aaatcatgat tattatgtga gaagctaaat gagttttagt |
| 53941 | gaatagatta tgagacttta gttatgtcta atttcatggt aataatattt ttttcattgt |
| 54001 | ttctatgctt ttaatatttt ctacttttct gtgttctatt gcacacatct gctaaagcta |
| 54061 | tttagaaata tgctcagaac aatttgtcgc agtgctgatt caatagatac aaacatgtag |
| 54121 | aaaaatataa gttacagccc atgttgaagt gccagtgaat gtacataaaa atgccaaaag |
| 54181 | tataattcaa gacatagcag cagagcttga cactgaaaga gaaacaactc taccttgcat |
| 54241 | tctggacaac catgacttga ctgcattgat tccaagagcg agagtgaagg ggtaaaacag |
| 54301 | gaaagtagaa aagacaggaa gaaaatagtt aggaacagaa ataagaaaga aaacatgaaa |
| 54361 | ataataacta gaggagetat cccataacat aaatgacatg aggcagaaaa tgctagaact |
| 54421 | acagtgaaaa taataccttg caagtgtaat acattccaag gcctttgctt gtctaataat |
| 54481 | taggMgtgtg attaaaagaa agacccagga attgaatagg ggtagcttat tagctgcctt |
| 54541 | tgagccaaac ctcaccaagt agggacgttt tatttgacaa attgcatgag gatagcttta |
| 54601 | ggtggggcat ttgttctcca tttcaccatg gttcccacca ttccttgttg cttaccccag |
| 54661 | aatggactga cagattcatg ttacctgcct gtccttcctg gttctttaag tttgtaacat |
| 54721 | tctaattgac acatatcctg taataaataa aagatagata ctcccatatt gtgtaaaaga |
| 54781 | tgatattcca ggatatcatt agagaatata gaatataaac aacaatgctt tattttcatt |
| 54841 | ctttcatttg caaatgctta ttacagacaa atccttggca gtgatttccc cttgtatcat |
| 54901 | aatattgtca taaataacat tgaaggtcta ataaatcaaa ggcggaatga agttatcagt |
| 54961 | ttgttgttgt tgttagactc taaaagatat tgccaaatta tagttacaac tactatatat |
| 55021 | agatataaat gaattaacat aaaacaaaaa ctaccttttc ctatactatg tgactattgg |
| 55081 | taatctctac ataaaattat tggtttttca aaatgttcaa taaagtaaaa ctagagcaac |
| 55141 | cataccaaca attccaagtt tttaaaataa aattattgag aaggatgaaa ttaaaagctg |
| 55201 | tgaactaact ttgatatagc acctggcaat ttaaaaatta ttttctgagt attatctaat |
| 55261 | ttggttctcc tgacaagcta aaaatgatga ttctgcctac ctcggctctc agggttgaca |
| 55321 | agaatactga atctcaaaga aattggtgat ctgcctgtgg tgatataaat actaagcagc |
| 55381 | agattcaagg gcagtgtata gctctaattt ggatcctgtt taaacaaacc aactacagta |
| 55441 | aaagaataca cttcatataa ttaaaatgct tttaaatttg ccacagatta ttttatgtcc |
| 55501 | tagaaaacag tctattttgg tgaatgctcc atgtgtactt gaagggaata tgtaatttgc |
| 55561 | tattgatggg tagtatattc tgtaaatacc aattagatca tcaaaccagc taggaaaata |
| 55621 | ttaagaggca accaaacaat tatttgaaaa atttaaaatt atgcttatac gaggtattaa |
| 55681 | ggctattttt aaatgagttt tatttttcag aaatatatac taaaatattt gcagaaataa |
| 55741 | ttataatagc tgggatttac ttccaagtaa tctggattaa gggagtgtat gaggaaaaga |
| 55801 | aacatgattg gtcactaagt gataactgtt gaggctgagt gatgggtaca tggtgtgtca |
| 55861 | aattattcct tctacttgtg taccacaatg gaaaatttcc aagttaaaag gaaaattcaa |
| 55921 | atatcactcc acaaattaaa atgaaaatga ataaaaaata aaaccagttt ctttaaatta |
| 55981 | ccaactcaaa gttcttttta ttttacctcc atttgttgat cttatgtttc catcttgcta |
| 56041 | atttggctga tatgaattgc tcaagtgaag gataataatt tgtagggttc aaatcagtgc |
| 56101 | tttgaaacta tccttgagta ctgacttata taccagaaca cagcaatttg cattatgtga |
| 56161 | aagttaaata tctccaaaga acacttgctt atatcaacaa tttatacatc acacaagaga |
| 56221 | ttcaaagaga tattttttat ttatttacca aagtcaatac ttcaaatatc actttgtgct |
| 56281 | attttataat ttttaaatca gtgatttgga aatatgtgta taaataagga tacatcaaga |
| 56341 | agccacttaa tcaacactaa cataatggaa aatacacatg tcctataaaa ataaacagta |
| 56401 | acttgtaagt ccttgaatat tgatttctca gataaatttg ggaaggtaag ccaattcttt |
| 56461 | aactttgaca caaataattc cttaaatgat ttcatagtga atctgatagt tatcacagga |
| 56521 | gtggtttcca gaattgaaag gctgatttgc tctcttggag gctggtttca ttatgatcac |
| 56581 | tgttcttcta caaccctgta aggcttaatt acaagttttt aattccacct tatctactaa |
| 56641 | ttggaaataa ataaatatga catgtggagg aaagtaagga aaagataata aagtcaatta |
| 56701 | aaatgaggtg tggaaattaa agagtagaaa atactatact attgttttaa agcatgagat |
| 56761 | gaagtacttc ttaattttac ttaccctctc cctggaagaa ggtacacttt cacaaaaggg |
| 56821 | tcagaataac cattgttgtc tcgaggaaca agatttcttg cttggagaat atgtattatg |
| 56881 | agatttccaa gatcatagtt aatttgaagc tttaggagag agaaaaatat tccataagtt |
| 56941 | tttaaaagca tgttatacat tatcattcaa tatttactag ttttttttaa agaaaattct |
| 57001 | tataagaagc atcaactttc attttcattt tatttgaaac caaacagacc agttaatgtc |
| 57061 | taatttaatt gagagctccc aggcatattt taattcctag caaacatgct gttcattctt |
| 57121 | tccacagtga aaaggcctgg cctgtttaaa agtttattta gaaggaaaat cataaggtag |
| 57181 | aatgctggtt atctttttta attaaattgc tactcttagg aaaatagtaa atgttgttaa |
| 57241 | tgtttattaa caacaattcc tagaattttg aaaagaaatt aactaattgg taaattatta |
| 57301 | taaagtatta tttgcatttt gccaaataat tagcagtggg gtggaactgt catttaaaga |
| 57361 | taaagctgtc agcactgaaa tgtaagaata tttaagaata cacagattat tactttttaa |
| 57421 | tgttatatta tctgWtatta aacctataga tacctttcgg taccagcttt cctaatagag |
| 57481 | tgagatacta caatactctt tttttctttt aattttagaa gctgaaaaaa gaagagacat |
| 57541 | tgctttatag atgcttattg gctgctgcac aattgtggct gaatactaaa agacataatt |
| 57601 | caaacaaata tttgagaaaa ctgcRtaatt caaaataatt gagttaagca tttttttctt |
| 57661 | ctacaattga aagactcttt tggcatcatt tagaataaag cttaattctg tttgatgtta |
| 57721 | cttttgtttg ttaaataata gagtggaagg gatccatatt tcaaagggca aatccatata |
| 57781 | gaaagatact aacttttaaa ataatccata taaaacagat atgctgcatt tatatttaaa |
| 57841 | ataggataac tcagatgagt catactttcc tgtaaaactt cataatgaaa taaatcctca |
| 57901 | ttttagtata attttgatct tattatgttt cctgtcagaa aatacatatt caattgctta |
| 57961 | tctccatcca gaaaagacta gagtccctga ccataataaa agcacagtga aaaagaaagc |
| 58021 | aaagtaccat taaagaagag gagaaagggg aagtttagaa ctctatgcca tcatgcagca |
| 58081 | actgtatgct ataatctggt actgaaatac agaatataaa ataacctgta attaatacca |
| 58141 | ctgaaatttc aaacaacttc ctgatcttta tttaaggaga aacacttccc cattctctcc |
| 58201 | tttctctgcc agccttagaa tccactttta agtgatgaag cacccaaacc gtttcttatt |
| 58261 | gaaatgaaaa cttgcaggcc acccgaagag agtgcttttc aaaatttgct ctgtatggcc |
| 58321 | ttagggaaca tctagagtta ctcagggtca gctttgagag agaatgggat gccccagcaa |
| 58381 | tcttcaatca cagcgactcc agtgctgata gattcatggt ttggttgtct acataagatt |
| 58441 | tcatttgaaa tgagcctttg ctaaaataaa ataataataa taataattga aatcactgcc |
| 58501 | ttacgaaaat atcccccaaa ttaagaagga ataatagtag ggtaaatttc aactacagaa |
| 58561 | ttacatctta ctccttaagg gaaagcaaaa taaaattaga aaaaacaaca acaataaaaa |
| 58621 | caaaaaacac agtccaaaag aaacactagt ccaaggacag aagtttaaaa atagtaggaa |
| 58681 | agagaaagaa ggctattaag aatcagaaat tctagcattt tctttgctgt ttgtgaaatg |
| 58741 | aagaaagatc aagtcagtga aaaacaggct ttctggaagt tggaaatagc agagaaacag |
| 58801 | ccggatctgg agtacagagg tcatcagggt agtactgtta gtttgttctt agtaacStgt |
| 58861 | caaccttctg cacattttct ccatttccac gctcacactc cacttctgct ttgtagtagg |
| 58921 | ttcctggata atcacttaat tgatttcttt gcctcaatgt cctcccttta taatatttct |
| 58981 | tgtaggtcac tgccatatga atttttatta cacattcttt tcaaatagtc tctctcttac |
| 59041 | tccaaaatag agatcatggt atattatgta gtggctaaca ttcctaggat tgtgttcaag |
| 59101 | tttcttttta atttagtctt aacctacttc tcgaagctga ttcttcaacc attactctcg |
| 59161 | gacaacaata cctttaattg tactctttct tctccaggaa cataatctcc ctttctttat |
| 59221 | gcttatcttc aaattaattc gagtttctgt tatcctttgt gaagtctttt gtgaaaattt |
| 59281 | cctctagaat gtattagaga tgcagctaag actctctttc atctgaaaca tactgttttc |
| 59341 | aattagttac tattttatag tgttaattct ttctatatgc tatttcttat ttagtctaaa |
| 59401 | ttacttcttg gtatcaccta aggtgcaggt tttaaagttt tatttttact atgacccaca |
| 59461 | atcagaaata tataaatatc aaaacacaga gagaatttaa aaagatgatt atctttttat |
| 59521 | ttttatatat ttttctttat ctatttttat tttattttcc tgatgaaaat taattttata |
| 59581 | agttacaaag cagttatgac ttatgctatg aaaaatactt ctctacaaaa actattcagc |
| 59641 | aatttttaaa gcaatttttg attaaaagaa gaaaaaatcc cagtagactc atgaaactct |
| 59701 | taaagaggaa acttcaaatt tccaggagaa tctgtcatgt ttaaaataca ttcgacaaat |
| 59761 | ataaagtaac tgaggcacaa aacatgttga cactgctaag atatcaggca gatttcaaag |
| 59821 | tatagaaata catagctatg attggacttc ataaaacata tatttgtatt aatatactat |
| 59881 | tctataaaat tgcatttttc ctttacagtt ctgtatatat tacagaataa gaaaaatcct |
| 59941 | aacagtgtgt ttacaatttc aacaaaccag tggcaaaaca accaaaaaag gttatataat |
| 60001 | ggcgactttt taaaaaactt catacctgaa tttctcctgt aattggatga gagacaacct |
| 60061 | ttgttccatc ggtaggctgt aatattaaag aacatatatt acattaatag atacattttt |
| 60121 | cacataattt atcatttcgg cttccttctg aaattagttt ctatttggtc agagcaactg |
| 60181 | cttggaaaat atattttatc catttggctg cttcagaata atggtgtcta cttacggttc |
| 60241 | tcaatacaca tttaatttta cagatgcata aatctatttt tcattttggt aagccggctt |
| 60301 | tgaagctgta attttatacc agcagtattt tcaattactt aagaaaatga acactttagg |
| 60361 | tttaaagcac ttaattaaag tatctacagg ttttaaagat ctatacagta atgtttaaaa |
| 60421 | tacattcaac aaataaaaga ttggtttaat acaagaactg ccgaggagca cagaaggtag |
| 60481 | aaaagtcctt ctttcacttc tttgctgaat gctcagcacc tccctgctcc cttccagagt |
| 60541 | gaatcaccca ctcaatcact ttccctataa aaattccaaa gaaattttta tagaactagt |
| 60601 | aaaacattcc taaaattcat ttacagccac aaaagaccct aatagccaaa gcaattttRa |
| 60661 | gctaaaagaa caaagccaga ggaattatgc tacttgattt caaaatctac tacaaagcta |
| 60721 | tagtaatcaa agcagcatgg tatggcWtaa aagcagacag acatatggaa tggaacacaa |
| 60781 | tagacaaccc ccaaataaat acattcattt acagtcaatt gactttcagc aaagatgtta |
| 60841 | agaacacata atgggcaaag gatagtctct ctaataaatg gtaccaggaa aattggacat |
| 60901 | ccacatgcag aggaataaac ttagatcctt atctcacacc atatacaaaa atcatctcaa |
| 60961 | aatagattaa agatttaaat ataagacctg aaaatttaaa attattagag agaaacataa |
| 61021 | gggaaaagct ctatgacatt ggtctgacca aagatttttt tggagatgac tccgaaagca |
| 61081 | caggcaacaa aagcaaaaat aaaaaataga agtacaacaa actaaaagtc ttttgcatag |
| 61141 | ccaatcagct tctgctgatt gtttcaatca acagagtgaa gagacaacct atagaatgtg |
| 61201 | agaaaatatt tgcaaaccgt acatctgaca gggtgttaat atccaaaata tacaagaaag |
| 61261 | tcaaacaact caacagcagg aaaacaaata acctgaatta aaaatgggca gagatttgga |
| 61321 | atagacattt ctcagaagaa gacatacaaa ggactaagag gtatataaaa aatcaacatc |
| 61381 | attaatcatc agagaaatac aaattaaaac cacaatgaga tatcaactca ccccagtcaa |
| 61441 | aatggctgtt atccaaaaga aaaaagatag tgagtgatgg tgagaatgtg gagaaaggga |
| 61501 | acccttgtac actgttggtg ggaatgtaaa ttgatatatc cataatggaa aacagtatgg |
| 61561 | aggttcctca aaaaaattaa aaatagagct accatatgat ccagcaatct cactgctgag |
| 61621 | catatateca aaggaaacga gatcagtatg tcagagatac caacactctc aggttcattg |
| 61681 | cagcattatt cacaatagcc cagatatagt accaacctaa gtgtctatca attgatgatt |
| 61741 | aggtaacgaa aatgtgttac atatacacaa cagaatatta tttagcctta aaaaacaagg |
| 61801 | aagtcctgtc atttgtaaca agatggataa acctgaagga cattatgtta agtggaataa |
| 61861 | ttcatgcaca gagagataaa tgccacatta tctcactcat atgtggaatc taacaaagtt |
| 61921 | gaacttatag aagcatagag aagaatggta gttaccaggg gctaagagaa aaggaaggat |
| 61981 | tggaatgatg gtcaaagggt acaaacttct cttagaagga agtcaaaagc tctgttatac |
| 62041 | aacatggtga ctacagttaa tagtaaatgt atatattctt caaaattgtg aagagagtag |
| 62101 | atttaaaaaa aattttttta agccaaatat ttcagaggaa tgaagagtag atttcaagtg |
| 62161 | ttcggaccac aaaaaaataa gtgaggtaat ccatgttaat tagctcaatg tagccattcc |
| 62221 | acaacgtatt catttttttt ttttttttgg agacagagtc tcactctgtc ccctccggct |
| 62281 | agagtgcagt ggtgtgatct ctgMtcactg caactccgcc tcccatattc aagtgattct |
| 62341 | cccacctcag cctcctgagc agctgggact acaggcatgt gccaccagac ctggctaatt |
| 62401 | tttgtatttt ttggtagaga tggggtttca ctgtgttggt tagggtggtc tcgaactcct |
| 62461 | ggtctcaagt gctctgcctc tcctggcctc ccaaaatgcg gggattacag gcatgagcca |
| 62521 | ccatgccagg ccacaatgta tacatatttt aaaacatcat gctgtacagt ataaacatat |
| 62581 | acaagttttg ttaattaaaa ataatttaaa atttaaaaat ctaagcaata ttgcatgaga |
| 62641 | tggttaattg cttaatttaa aaagaagttg tcttcaaata tgttcaaatt gtatttttat |
| 62701 | aacatagaaa catctacaaa acagaattta gtattaatta tgttggtatt ttagttggct |
| 62761 | agtttatgaa ttatttttaa atgattatca aaatagagcc tctataaagt aattattcaa |
| 62821 | aattagcaca taattgcttg tggtaaaatg tagttgtgtt aaaatttatt ttaaaattta |
| 62881 | gcctaactgc ctttgaaatt actttgctta tgaagctttt ctatatttag tttcaaacaa |
| 62941 | atcattttac gatgcaatga ttaaggtcaa cctacttttg ttgtttgctt ctgaataaaa |
| 63001 | ggattctttt tttggtgaca tctgctgtat ttttatagtt aaaaaacaaa ctttattttt |
| 63061 | tatgtctggt cacttgacct aagcatatac agcctgtcct tcccatatat ctcaccacat |
| 63121 | aaagacaaac ctttattcat ttttaaacaa aaggacactt ttttcgcatg tatttatcaa |
| 63181 | ctacagggat agtttttctt aagaatcaaa tctatgaggg aaatcataaa ttttgtatat |
| 63241 | ttcctctgct ttttataagg ttaaaaaata attttatgct tttttaaatt ccccggcact |
| 63301 | ttgaaacaat agcattggaa gcatttgttt cgaatgaaga tttgcatcaa taaaaaaatt |
| 63361 | cttaatatgg aattgaggat aaagttcaaa gtagtgataa catgttactg gttttccttg |
| 63421 | tttgttattg tcttaaattt tctttatcac tgtgtccttt ttacttttta cttaattata |
| 63481 | aaatctaatg taatatgtac cttttgttcc aaaaagatgc aaagctaata ttctatatac |
| 63541 | gtatctcaga ttcatgcaac aacaaagaga accagaaaat ataaagtatc agagacattt |
| 63601 | ttcaagttcc tcataataac tacKgaaata tttaaaagat gtagaaatac atcatcttga |
| 63661 | tacatcttga tcagttttgt actcagaaga gaaaaaatgt ttcttgtttt gatagatcat |
| 63721 | ctggaaatga gaaatcataa gaaaaatact ttagaaaatg atattttatt ttctgtctct |
| 63781 | atgctgaata aagagctaga aaacaagtgg gtcagaggtc acatcaacaa tcctcacctt |
| 63841 | gctgctgccg tgcttctttt tgctcactga gggggaccct ggttgcccag ggctgggaac |
| 63901 | ggaactggat cctgctgatg taggacctga atgaacatga gatccttttt caacaactga |
| 63961 | tgacagaacc agcggtgact gctgtagtga aaccttctgg agttctgctg ccaactgctt |
| 64021 | aggatcaacc cctggggatt tcgccttatc cactacaaca aaatgaaaga gatttacata |
| 64081 | cttctccatt tcataggtac tttataccat ggaactatgt gagttctagg tgacaaaggt |
| 64141 | aaaacattac ctataaaata agctaaactt taaaaatgaa attaactcta ttgtactttt |
| 64201 | atgtatcaat ttctaaggaa gtctacaagg ctctgagaag agtgacataa tgctgaatta |
| 64261 | cagaggaata gtgaagttcc cgttgcatct tcagaaaaat gtttttaata ttttacataa |
| 64321 | agaattgttt gaatgatgca atatcctatt ttatccattg gaacttaatt tgtatgatta |
| 64381 | gctgtttttc cttccttaaa ttttacatag ttctttctct aaggggaaaa ttttctaagg |
| 64441 | ggaaaatttt aatgggttta tttatattta caaatgtatt aaaattcgta tagcacttgg |
| 64501 | tctattctgc ttagtacaat aacccagact actagaattg tggctgtcag agtaggaagt |
| 64561 | gctcaacaaa atttattaaa cataggaaaa agtgatgttt ttaatcttac aatggattta |
| 64621 | acaaatttgt agRttatatt tgttattcaa gaaagagtta aatctatctc atcatgtact |
| 64681 | tatgaaatta catgttgatt tttgtttact ataaaaaggc aatgaaagag ttcaggaata |
| 64741 | attttttcaa aaacccagtt attctaaaaa gaatcaagta cgctctaaaa attatgtaaa |
| 64801 | accaaaaWga aatggctcca agcattactt tcacaggatg aattcatatt taaagaatta |
| 64861 | aaactgtgaa ctcatgcagc aaggatttta cgtgaacttg tttaagggga ctaacatgca |
| 64921 | aatatgtatt tccccagaat gctaacactg tagctacaat gccctagatt ttgtttataa |
| 64981 | ttatataaat acataaaaaa atctatccat aactttatat ttgtctatat ctgtgtttNt |
| 65041 | gtctacatct tcatctacaa ttacatctgc ctctgcaatt acttctattt ctatctcttt |
| 65101 | atttacagtt gaaactagat ttcttttacc taccagcttt tggtggctca tgaagttcca |
| 65161 | gatgctggga attttcagaa tctgatagca tattgaggtc cctaaaaatt aaaacaaaac |
| 65221 | attaagaaag atattgagga aatctgagac aaagagcact tttttccaac tacccttaat |
| 65281 | ttttttcata ggtaaatgag aagtggttaa tgatataaag caactccaaa gcatgcctac |
| 65341 | catcctaacc caatatatat ttgcaatgca gttaaaagtc agagctgata aaaatgatta |
| 65401 | tttaaggtga aatctttatg aattaaatgt aagttcattt ttaaatacat atccatgaaa |
| 65461 | tctaaatggg ttcaggcttt catttaaatg aagcatgttt ccataaaagc atgaaaggga |
| 65521 | agaggctgtt gacacaatca gaaactaagt tagctgaatt ctttctatgc tttaggcttt |
| 65581 | gattcacttt attttataat tccagaaaat cttgctaact ggcaaaggtt ccaccttaac |
| 65641 | aattttaaat taaaagagtc atggatagcc aaaaaatgga aacagaaaga tggggaaaaa |
| 65701 | ctcacagtct tacacatatt tctgcttccc cactttgctg actaatgata ctctgaactt |
| 65761 | cttcatatgt tttagaagtc aagggaattc cattccattc caatacttgc atccctagaa |
| 65821 | agacaaattt gaatataaaa tcaaacgtgt gctatctcta gtctgggtac atatggcatt |
| 65881 | gaagacattt atttgcattt agaagtcaac tcagcacacc attttagaga acatcagttc |
| 65941 | caagttctct gcaacattaa taccaaaacc attatgtagt ttgatctcat taacttacct |
| 66001 | gaacacataa aatatattta atacaacatg caaaattttg cttttgaaaa tctgtgtaat |
| 66061 | attatcaaac gcatgcattc tgttcacact gccataaaaa acattattga atatatattg |
| 66121 | ctcagaccaa ttttgtatag gaaacaaaca aacaaacaat tagaaacccc aatataattt |
| 66181 | tcctgcgggg aacaagctac tagaagaatg tatttggtac tttatcatga aatgggagca |
| 66241 | gtagaaaaaa gtcatttgga tttctccatt tcagaaactg ttgtagcttc tattttgtcc |
| 66301 | agatgaaaca gaatgccact tcgtgtcttt ttcttcacgg tatgccagac atccctgaat |
| 66361 | tgaggaatag caggaatgtt ataggaaggg cagctgcagg ggtctgtgac ctgtgcacta |
| 66421 | gtacaaggcc ctgtgctcag aaaggcctat gcttggttta atgctctgct gttgtcatct |
| 66481 | tgacattctt agtaacattt gaacaaggaa cccacatttt aattttgcac tgggttttgt |
| 66541 | aaattatgta gctggcctgg cctgacctgg tcagggcatt tcatttttcc tctctatcta |
| 66601 | gaaagataat ttcatagaaa aaagaaaagt gtaacataat taatatttca atattttaca |
| 66661 | tctagctcct ttggtccatt ttatatgacc tgtcaaaatc attgaggggt ccacgtgtgg |
| 66721 | catgattcac attgtcctga catgcagcta tgtgaactgg gtacagtaga tgaacccatc |
| 66781 | caagatagtg ctcaaatatt tgtgaacttc accagcttta tttgagaaaa atgtctatat |
| 66841 | accacaatta tgttgttgaa ccattagtta gttttttttt tttttttttt tttttttaRa |
| 66901 | caggtcccac tctgtcgccc agactggagt gcagtggtgt aatctctgct cactgcaagc |
| 66961 | tccgccttcc aggttcaagc aattctcctg cctcagcctc ccaagtagct gggattacag |
| 67021 | gtgtgcgcca ccacgcctgg ctaatttttg tatttttagt agagacagag tttcaccatg |
| 67081 | ttggccaggc tggtctcaaa ctcctaacct caaatgatcc gcctgcctca gcctcccaaa |
| 67141 | gtgctgggat tataggcata agccactgtg cctggcctga accattaggt ttttaatgag |
| 67201 | cttcacagat gcctatgtga aattacctat tgatcatcat tggctccaat gggaaattct |
| 67261 | aaacatggca tgattgtata tttaaaagtt ttgatgatta tgatatatat tagaaagcat |
| 67321 | attaccagtt gcaagtaagt aaaggaaaaa cttttacact atagtcaaat ggtaaaagaa |
| 67381 | ttccataaaa aggctcacaa taacacaaac aagaatgact tttctataac ttactccaag |
| 67441 | gaaactttat cttcagatgg gccttaggtt tatttcaaca atcccaggct gttgtacata |
| 67501 | taaaatgaga aaattatgta aaagacagtg acaacaacac taaataaaaa tatagcacgc |
| 67561 | tgaaagagga aaataaagca ctttttttaa ataaggagga agagtacatt aataaacaga |
| 67621 | tgattttgaa ttcaatcaaa ttaaagttat tatatttcag aaattKcatt gtcacatttc |
| 67681 | tgcaggagca catactatga aaaggaatag tcacatattt tcatgctttt tctgttgaat |
| 67741 | ttacctgttc attcttcaca tcccaaaata aaagggcaaa tttacaaaac aattatcctg |
| 67801 | taaaattaaa aaaatgcttc taaagaacta gtatggttaa taaaatctct taaaaaatta |
| 67861 | tacagataac tagggtctcg cctggatata cacctgacac cataaactac atgatctgct |
| 67921 | ccataccaca atgcaccaat ggaaattctt tttgcgccac ctcagtttcc caccctagtt |
| 67981 | gtttcagtgc tgtagttcaa aagaatggga caattctttt atcattgctt gttgcctaca |
| 68041 | gtgagacacc atagggaaga tgaccactga acacagtcat ttaataatat tttccttttc |
| 68101 | tattgtctat cttcctacaa attctatgag ccatattaaa ataatgaatg atatacaggt |
| 68161 | ataacaaatc gctccttatt tatctcaaaa gaggttctag tagttgtgaa aattaaatat |
| 68221 | ggcatttcga atatatatga aaaatatttg ttaattgtaa cattatataa actatttaaa |
| 68281 | atgcatgcaa aatttgttga aaattattat gatctgtgga tgttcaataa tgtatcttgt |
| 68341 | catttaatag gtttgaatat aagcattttt agtctcaaaa cagcttttca cactgcgttt |
| 68401 | tagaaagatt ttctggaaaa tattacccat gtgaattatg atatKtatta tttagtatta |
| 68461 | taagtaaaca tttatatttt ctttacattt aaattactac aaatttcagg tatgaaagta |
| 68521 | cacagatgag acttaaattc cttgaaagta cctatttaaa tagcttatta ttattaNtat |
| 68581 | Ntatttattt tttgagacag ggtcttgcac tatcacccag gctagagtgc agtggcacaa |
| 68641 | tcttggctca ctgcaacctc tgccaaccgg gttcaagtga ttctcctgcc ttaggctcct |
| 68701 | gagtagctgg gattacaggc acccaccacc acacccagct agtttttgca tttttagtag |
| 68761 | agacagggtt tcaccatgtt ggccagcctg gtcttgaact cctgacctca agtgatccac |
| 68821 | tgacctcagc ctcccaaagt gctgggatta caggcgtgag ccaccatgcc tggcctttaa |
| 68881 | tagcttttaa taggaaatta agacagtctg gaRttttgct gaatatgtag catcgatgat |
| 68941 | aggatttgtK gcaaagtctt ttgcttaata ggMtactaaa cactgaaaaa gacaataatt |
| 69001 | aggtaattca agaacttagt attaacataa aacattatca aatatatgtt ttcataacct |
| 69061 | ctaggattac ttaatattgt atgctcctta aatgggcaaa agctattcct ctctttataa |
| 69121 | tacaaaattt catgttattt cttgatctat atttaatatt taccatcaca ttaaaaacat |
| 69181 | cttactgact aaaattattc agttagttaa aaatcttaca ggttactcta catccaaaca |
| 69241 | tatgccacac aaaagctcct gacgtctgtt cattttattc attccacatg tatttttcta |
| 69301 | acacttcata tgtggtaggc actacacaat tcatcacctt tgYttatgtc taacatttta |
| 69361 | tggtgttata aaaattaata aaaatgatag tctactagaa aacaattgga aatatctaag |
| 69421 | caattagtaa aataaagatc atteacacag gaggaagttc caaaactttt gcaagtatat |
| 69481 | gaagaaaacc ccagattcat tagtcattag aaagtgcaaa ttgaagccat aatatattac |
| 69541 | ctaactgtca tcatattggg acatatttga aaaccataaa atgctatgtg ttgattggaa |
| 69601 | tgtgggattg gtgaccttga gtgactataa atagataaaa caacccctta tttacaattt |
| 69661 | attaacatgt aattatttta atgagaatgc acatgagtgc aactgttctg gaaggcaatc |
| 69721 | tggcagaatg tagttaaatt aagcatatgc atactctatc acctaaaaat tctttgtggg |
| 69781 | tatgtatttg tgaggaggtg gaggtatcaa gagaaatagg cattgagagc attgagagtg |
| 69841 | ctgtgaacgc tcacgatgaa aatacttggc agaggctgga agaaacagac cataccaggt |
| 69901 | gttaacacca gaaggtggat ccatcctgaa gtctttgcca agttaaaaac aagtgagaaa |
| 69961 | caggatctga cctatagggc aatgttattt atgttgattt aaataacacg cgcacaaaat |
| 70021 | aaatcattac atattttatg aataaatacc aagcatctca taaattataa tagttgttca |
| 70081 | cagaagggag agcagaatgg gaaaataaga tagaggaaaa tgaatgtgtt aataaatgaa |
| 70141 | taaagaaatg aaaaaaatgc aggagcagac cttgaaaaaa aacaaaggcc caaacctaat |
| 70201 | ttaaaaatgt gtagtataaa ttatttgtaa tttgtttgct atcttttcca tactaagatt |
| 70261 | attaatcacg actataggtt aattttccaa atttgaacaa tcactctcag gagtcttcta |
| 70321 | taaagcgcta gcagcagcag caaaagatca gctgaacatg gccatctccc ctggcggact |
| 70381 | caacagcaaa cacatcttgc aagtggggtt caatacaatt gtccaaatca attatactca |
| 70441 | ttgctcattc attttaatgt taggcagtgt taatgtatga gaaagacata gatagagaag |
| 70501 | tggattttat tttttaaatt tatatatttt tcatatttta caattttgat ctatttatat |
| 70561 | attaacataa aagggggaaa ggtaagaagg atggaagaaa ggaaagaaga aggatcaaag |
| 70621 | aaaggaagga gtaaaaagaa aggaaggggg gaatctttgg taaaggaaaa aactagtatt |
| 70681 | tgaatagtgt cctgcttatt tacagaacaa ggagatacac tcagtcataa taagagattt |
| 70741 | aaactagggt atattttcct gagtatattt attttgaaat atacaataaa ctatttgtaa |
| 70801 | tggttaatat tgagtgtcaa cttgattgga ttgaaggatg cagtattgtt cctgggtgtg |
| 70861 | tctgtgaggg tgttgccaaa tcagattaac attttagtca atgagctggg agaggctgac |
| 70921 | ccaccctcag tctgggtggg taccagctaa tcagctgcca gtgtggctag aataaagcag |
| 70981 | gcagaagaag ctggaaagag ccgacctgct gagtcttccg gccttcatct ttctctagtg |
| 71041 | atggatgctt cctgccctag aacatcagac tccaagttct ttagcttttg gactcttgga |
| 71101 | tgtacaccag tggtttgcca gggactcttg ggcctttggg cactgctggc ttcctacttK |
| 71161 | tgaggttttg ggattcgggc tgatccacta ctggtttcct tgctcctcaa cttgcagatg |
| 71221 | gcctatcatg cgactttata ttgtgatcgt gagtcaattc tccttaataa actccctttc |
| 71281 | atatatacat atatcctatt agtgctatct ctctagagaa ccctgactaa tccactgttg |
| 71341 | ttaagtatat tcactctact gtgctattga acactggatc ttatttattc tctccaactg |
| 71401 | tatttttgta cccattaacc aacctctttt catgcctcac ttaaaatcct tcccagcctt |
| 71461 | tggcaaccat cattttattc tccatctgta tgagatcaac atttttagct cctacatatg |
| 71521 | gagtgagaac aygcaatatt ttattttcta tgactggctt atttcatcta acataatgac |
| 71581 | ctccaattcc agccctgttg ctgcaaatga tgagatctca ttccttttta tggctgaata |
| 71641 | gaattccatg gtgtatacag gtacaatctc tttacccatt tataaaattt tgaagttgaa |
| 71701 | aagtgttgtg ttgaagagct aatctcataa taatattgat attttttaaa ttattattgc |
| 71761 | tattattttt attttttgta ctcatggttc aaacaaaact taaaatagag cttcaatata |
| 71821 | caaaatatat aaaaatgggc ctactttcat caaagcaggc tcaatctgcc ttctagcctc |
| 71881 | ctcaccattc tccagatcct attctatggg tacccttaga cacacYctgt ttccctgatg |
| 71941 | tacatgagtt gtgagtttaa aataacataa caacctgaat aagggcataa gaacaattgc |
| 72001 | tattaactag gacactgttt cttgagatgt actggaagga aggtactctg aatttccgtt |
| 72061 | tattgaaaat gaaatcttaa ttggtttaaa aactataatt tttttaaacc caataactat |
| 72121 | aggaataagg ctttcaaata gagctctgtg taatatttac cattaaaaat taaaatttgc |
| 72181 | ctatctctca ataaacagaa tacacactat ttttatatag ataaatattt tctttatcag |
| 72241 | aaatatggtt tatgagccac cttgctgttg gtagctgact gttgacatgt tcttttcacc |
| 72301 | ctaattgaat tgtgttcaca tgtagtttgg ttatttacta gttgttgtgc ccttgagcaa |
| 72361 | gttggttaat cctccttcat ccttgactcc ctcaagggga ttatacttgc taggtgactg |
| 72421 | ctctagtttt atttccatcc tatgatagag aatacaatat gacagaatgg gaaacacaaa |
| 72481 | acagctgctt gtttacaact tgtaaatatg cattttttga gctgaaggtt tatataagaa |
| 72541 | atttaaaaaa tactttagga tcctaaaact tccattttgt tcttaattat tttccttatc |
| 72601 | tatagctgtt cttgacatat acagtaaaac ttccccaagc atagaaatca tgtcatctaa |
| 72661 | tttttctttt ttgcacttcc cacatctcct ctcatggccc tctcagtggc agttgtccag |
| 72721 | gttctttggc tttgtctttg ctcccctggg tgccaatggt gtccccctta ctgagtttct |
| 72781 | tttctctcgg cattgttaca ttgttatatt tgtaacattt ggattggtta actctgcatg |
| 72841 | ggacatccaa tctgcagtct gaatctaaat ttggtttaaa tctacatgcc aagcagacaa |
| 72901 | atatttatca ttaatgccac taacccaaag ttgatttaaa tgcaaaatac caaaatttta |
| 72961 | ctccatcata cacttaaaaa acatgtgata cgtcataatc ttatctcttc ataatataaa |
| 73021 | gataatataa ttttaaactc attaaaaatt ttaatgcaaa ttttataaat gaatattctt |
| 73081 | tcaaatttta gtagtattaa atatacatac acaggagatt tgcagtatca tcctactctt |
| 73141 | ttgctttaag tcactgtatt tttatattga atataaaatg aatgaaacag attgttatgc |
| 73201 | aaacttatat gctatgataa tatactctgt ttccattttt gggcaggact gttgacaaga |
| 73261 | attctttaga taaatgttat acagcatgga tatcttttta aatcgctcag agttagagaa |
| 73321 | ggtaaattct gtatttgaaa ctctcattga tttgggatgt gatcttggac aatttacttt |
| 73381 | ccatgtactt tctttcactc tcaaattttg tgaagaacaa gcaattaaaa ttagcaaagt |
| 73441 | gtggcttgca tatataaact ccaaaatgaa ataaatttac atatttttaa aacttttcat |
| 73501 | cacattttga ataaaagaaa tttaagaaat accaaagact acagttgaat ggtctgagct |
| 73561 | gactggtatt tcttttctag gtctgagtac attgaaagta tttagagtca atgacttctt |
| 73621 | tactcacata tctaaaatag atttagctcc aatcacaaaa aagtataatt gtaaaataaa |
| 73681 | cagattctta cttatatcac agtattaata gttaacaata aaaatattgt tcaaaaggat |
| 73741 | ttcatcgtca aatgtgttgt aaaagctcaa gtcacaacca ttttttaggc taatgttata |
| 73801 | tagtaagcaa gatacttata agtactgtag ttttaatctg ttatactata tatttaaaaa |
| 73861 | gtgacaccta agaggtgata taacttgtct aatatcccac agaatggtag tacagaaata |
| 73921 | tgaaccccaa ccatctccta ggccaatgca tgtatcagta aataaaaaaa ctaattcaac |
| 73981 | ttatgccaac taagcctaca tgctagccta cctgaaggca aactatgttc aaatatgtga |
| 74041 | aacttagcaa aaataacata tattatttgt aatgccattt aaattgcaac ataatatgat |
| 74101 | aatttggaaa ttttattaat tgcctacatg ctgatattat ttggaggctt ggacatgcaa |
| 74161 | aaactgaaga cattattgaa cgaagagaaa gttgctgtgg gatatatcac ttatggtgag |
| 74221 | taaacagtga cagaaatgaa taaaatttga ttgggtctct tatccctttt ccccatctca |
| 74281 | ctcccagaag agaagacaat gtcgtatttc tccagggaga ttgatgaaaa attatgctga |
| 74341 | atcttactgg gaagaaaaat aatgtaagaa ggatgaaaga ataggaggtg cagattcagt |
| 74401 | gctagggagg aaatggtggt tggaggttcc aaaggaagta gggaccagca tcctgcttgt |
| 74461 | ttatattgcc tatggaagat gtctggcata ttttttcaat aaaaattgcc aagaggtgcc |
| 74521 | tttagaaagt tgaacactag attctgtgac tctcaatgta ggcaaaacta ctcttgtgaa |
| 74581 | aacttgggca cagttaaggg agctactcat tatcaggaaa gtgtgctagc tttgacattg |
| 74641 | cactcttagt ggcgtggact gatgtacaga ggcccaaact ccagtgttct agaatgtgta |
| 74701 | ataccctcac tgggaagttc ctcaaggcat gtggtgcagg aaacaccaaa tgatgaatgc |
| 74761 | aattcaattt cctaccaaca gatgaaagaa agattggact tgattacatt tgatttacag |
| 74821 | aaaaagtaat atttcttata ctcaaatatg actgttgcaa agatatgctt tttaaacata |
| 74881 | cttagtgtga ataagggaat acaattagcc atgatttgtg gaccaacctg ttagaagatt |
| 74941 | atcatgataa gattcgattt ctaatcaccg tgaccactaa aacaatccag tccagtattt |
| 75001 | ccttgaagga ttggtataga tgccaaaagc cactttatgg ctacagttca ttgtatgtct |
| 75061 | cattctatgt gttatttgga acagactgaa tcagaaagtg gctgaatgag gcagaataag |
| 75121 | ggatatgagt atattagtat aatttgattg gcccctagaa aataatgtta catggggagt |
| 75181 | tatccaataa tagatatgta atgtcatcat ctcacttgaa gtagattgca gtttataagt |
| 75241 | ttataacttc ttcctagttt aatatcacaa tttatttgtt agaatgctca gaatggatgt |
| 75301 | tttttactaa ctttctaata aaatgatatt aacagattaa tactggtctg ctgtggtttg |
| 75361 | aacgcctcct caaaaactca tgttgcaatt taattgccaa tgttatggta ttggggggtg |
| 75421 | ggtagggcct ttaagagatg atgaggccaa gccctaatgg atggattaat gccattattt |
| 75481 | caggagtggg ttagttatcc caaaagtgag ttccagatac aaaggatgag ttcaacctga |
| 75541 | tttccttact ctctctgtgt tgtatgctta cttgaaagag catgcctctt cactatttta |
| 75601 | tgacacagca agaaggtcct ctcaagatgc cagcactgtg atcttagact tatcagcctg |
| 75661 | aagaacttta agaaatgaat ttcttttctt tataaattac tcagtctctg ctattctctc |
| 75721 | atagtagcag aaaacagact aaaatgctat caaatcttat gaataccata atcatttaac |
| 75781 | aataacaaac taaaccaata ctatatggat tgtgtagaaa tccacaatta attgaatgga |
| 75841 | cttaggaagc tagggtagtt gatgcactta ggaatgagaa atgcttgcct taactgttgc |
| 75901 | ctcctctgct ttaagtattc ttactgaatt attaataatt taacttcagt tatgttgata |
| 75961 | acctttatga tattcatata ttctgtactg ttctttttta actgttactt aaatgtatct |
| 76021 | tactactcag atgatagatt ttaacataaa atatcacttt atactaggaa agtgtctcct |
| 76081 | aacaaatata aatgctatct gaactagtgg caaaatcatc atcactgcca ccaccatctc |
| 76141 | cactgtacat agatataagg acaatataaa tttcatttta taacataatt tgacagataa |
| 76201 | aatacccaga agtttgcata gattactgag gtataacttt aaagcatgtg taaaatgaga |
| 76261 | taaaatggaa atcatgtttc tgaaaaagaa agttagaaga aagaaatctc tcaatcaaaa |
| 76321 | tttaagagta aaaggaaaat tcaaatatat tcatcctata gtataagaaa ataaaatgtt |
| 76381 | tcttttgaca aaggaggaga gaggttttat cacaacccaa atattaaaag tatgcttcac |
| 76441 | aagaaataat acaacatggt caattgttat gtataaaaat gttaattaaa gattaatatt |
| 76501 | ttaattgaag aggaagaaat acttccaaac tcattgtata aggccagtat taccctgata |
| 76561 | ccaaggccag acaaggatgc tacaaaggaa aattacagac cattatccct gatgaacata |
| 76621 | gatgcaaaac ttctcaataa aatactaaca tactgaattc aacagcgcat taaaatgatc |
| 76681 | atccaccatg atcaagtggg atttatcatt gggatgcaac aaggtttcaa catatggaag |
| 76741 | tcaatacacg tgctacacat taaaaaaatg aagaacaaaa agcatatgat catctcaata |
| 76801 | aatgtagaaa aagcatttga caaaattcaa catcctctca tgacaaaaaa ctctcaacaa |
| 76861 | attgtgtata caaggaatat acctcaaaat gttaaagtcc atgtatgaca aatctgcaac |
| 76921 | taatatcata ctcaatgaag aaaagttgaa agtttttctc taagatgttg gtatccattc |
| 76981 | tcactaattc catcaacata gtactggaag tcctacccag agcattggga aagaaaaaga |
| 77041 | aagaaaatac atctatactg gagaagaaaa ggttaaaatt gtctatttgc agatgacacg |
| 77101 | atgttatata tggaaaacag tcaagactcc accaaaatac tgttataatt aataaacaaa |
| 77161 | ttcagtaaag ttacaggata taaaatcaac aacaaaaatc agcctatact atactcagct |
| 77221 | tatactatgc acttctaaac actgacaaac tatctgaaaa agaaattttg aaatatcata |
| 77281 | tatgatagct acaaaaagaa actatttggg aataaattta gccaagaagg tgaaaaatct |
| 77341 | atgcactgaa aatgataata ctgatgaaag aaactaaaaa agacacadat aatagaaaaa |
| 77401 | atactatgcg cctatggatc agaagactca atattgttaa aatgtccata ctaaccaaag |
| 77461 | tgatctgcag attaaatgca atccctacca aaatttcaat gtaatttttc atagaaatag |
| 77521 | aaaaaaatct tgaaattcat gtggaaccac aagagatatg gtgtctatgg ggtgaggctt |
| 77581 | ggctcctctg aaaaggcagg gaagagtggg aaggattgcg tttggttgtt tgagggccag |
| 77641 | ctcagctgca atacaataga acaccaggta gacttctaag atttttgact ctggttcctg |
| 77701 | actcctggat ggcacttctg gacccacctg gggcctggtg gacctcacca ccctgaaggg |
| 77761 | aaagacaggc ctggctggct ttgccacctg ctaattgtag agttccaggg ctgtgagtga |
| 77821 | acataggcaa tagacaggaa gtggttatag caggccttgg gcaagatcca gtgctgtgct |
| 77881 | gactttaggt cttacccagt gtagtcacag tgatgttgcc cacaggggtg cttgtgtcac |
| 77941 | tcaaccccca gctttaggtg gctcagaaca gagagagaca gacgttatgt ttgggagaaa |
| 78001 | gtaagagaag agaacaagag tctctgtctg gtaatccaga gaattctccc agatcttgtg |
| 78061 | ctacaccacc aaggttgtac ctttacaagt ctgccagaac cacagcatta ctgggcttgg |
| 78121 | ggtgccccct aaagcagaca tagcttagac cataacacca agtcatttga aatatctgga |
| 78181 | aagccttccc aaaaggacag ttacaaataa gcccagacag taaagactac agtaaatacc |
| 78241 | taagggtctt caatgcccag acaggaaaac atgacctcac ctaatgaact aaataaggca |
| 78301 | ccaagaaaca atcctggaga aacaaagata tgtgaccttt tagacaaaga attcaaaata |
| 78361 | tttgtgttga ggaaatgcta agaaactcaa gaacacagag aaggaattca gaattctatt |
| 78421 | agataaattt aatagagtat gaaataacta aaaggaatca agcagaaatt ctggaggtga |
| 78481 | aaaatgcaat agatgcaact gaataatgca tcagaggtct ttaatagaag aattgatcaa |
| 78541 | gcaaaagaaa ggattagtga gcttgaatac aggctatttt aacgtacacc aagaagacaa |
| 78601 | aaaaagagag aattttaaaa ataaagcaca cctacaggat ctaggaaacg cctcaaaagg |
| 78661 | gcaaatctaa tagttattgg ccttaaagag gaagtagagg aagagattgg ggtagaaagt |
| 78721 | ttattcaaag ggataatgac agagaatgtc ccaaacctag agaaatatat caatatccaa |
| 78781 | gaacaagaag gttttagagg gccaagcaga tttaacccaa agacaactac ctaaaggcat |
| 78841 | ttaataatca aactcccaaa ggtcaaggat aaagcagcaa gagaaaaaaa aataacatac |
| 78901 | aatacagctc caatacatct ggtagcagac ttttcagtgg aaaccttacg ggccagggag |
| 78961 | agtgatatga cttatttaaa atgctgaaga aaaaaatctt ttacgttaga atagtatgtc |
| 79021 | tggtaaaaat acccttcaaa catgaaggag aaataaatac ttaacccaga caaaaaaaag |
| 79081 | ctgagggatt tcaataccag agctgtccta caagagatgc taaagggggt acacttcaat |
| 79141 | cacaaagaaa aggacagtaa tgagcaataa ataatcacct gatggtccaa aattcattgg |
| 79201 | taatagtatg tacacagaga aacacatatt attataacgc tgtaactgta gtttgtaaac |
| 79261 | tactcttatc ctaggtagaa gattaaatga tgaaccaatc aaaaataata actacaacaa |
| 79321 | cttttcaaga catagcataa taagatataa atagaaacaa caaaaagtta aaaagtgggg |
| 79381 | ggatgaaatt aaggcataga gtttttatta gttttctttt tgcttgtttg tttatgcaaa |
| 79441 | tagtgttaag ttattatcag gttaaaataa tgggttataa gatagtattt gcaagcctca |
| 79501 | aggtaacctc aaaccaaaaa gcatatgata tatacgcaaa aaataaaaag caaaaactaa |
| 79561 | attatatcac tagagaaaat catcttcact agaggaaaac aggaaggaaa gaaaggagga |
| 79621 | agagaagacc agaaaataaa caacaaaatg gcaggagtag gctctaactt atcagtaata |
| 79681 | acattgaatg taaatggact aaacacttcg atcaaaagat atagagtggc tgaatgggtg |
| 79741 | aataaacaag acccattgtt ctgttgccta caagaaatac acttaacctg taaagacaca |
| 79801 | catagactga aaataaaggg agggatgaag atatttcatg ccaatgtaaa ccaaaaatgg |
| 79861 | gaacctagaa attaataaca ggaggaattt tggaaactat tcaagtacat ggagttatta |
| 79921 | cacttagaaa aagtagattc caagacaaaa gctataagaa gagacaaaga agttcactat |
| 79981 | ataatgataa agaggtcaat tcagcaagag aatataacaa ttgtaaatat atatgcaccc |
| 80041 | aacactggat tacccagata tgtaagggaa atattgttag agctaaagag agaggtaggc |
| 80101 | tccattacaa taatagctgg agacttcaaa accccatttt cagcagaaaa tcagacaaag |
| 80161 | aaacatgaga cttaatctac actatagacc aaatggatcc aatagatatt tatagaacat |
| 80221 | ttcagccaag agctgcggaa tacacattct tttcctcagc acatggatca ttctcaagga |
| 80281 | tagaccacat gttaggtcac aaaacaagtt ttaaaacatt caaataatgg aaatcaccaa |
| 80341 | gtattttctc ttactacaat gaaataaatc tagaaataac cagaggaatt ttggaaagta |
| 80401 | ttcaaatata cagaaattaa acaatatgct cctgaatgac cagtggatca ataaagaaat |
| 80461 | ttagaaattg taaaatttct tgaaacaaac gataatggaa acacatacta aaacccatgc |
| 80521 | aaaagcagta ctgagaggga agtttatagc tataagtgtc taaatcaaaa agagaaaaaa |
| 80581 | gttgaaatga aaaatctaat gacgcatctt aaagaactag aaaagcaaga gcaaaccaaa |
| 80641 | ccccaaatta gtagaagaaa acaaataata aagaacagag tggaaataaa tgaaatcaaa |
| 80701 | atgataaaaa tacaaaagct caacaaaacc aaaaattggt tttttgaaac agatatatga |
| 80761 | aaaggtgctt aacatcattg atcatcagag aaatgaaaat caaaactaca acaaaatata |
| 80821 | atttcacccc agttaaaatg gcttatgtcc aaaagacagg caataacaaa tgctgatgag |
| 80881 | gatgtggaga aaagagaacc cttgttcact gttggtggga atgtaaattg ctacagtcaa |
| 80941 | tatgcagaag agtttggagg ttcctcacaa aactaaacat tgatttatca tatgaaccca |
| 81001 | caatcctact gctgggtata tatccaaaag aagggaaatc agtttattga ggagatatct |
| 81061 | gcactcctat atttattgct acactgttta caacagctaa gaattggaag caaccagagc |
| 81121 | aagactctgc ctcaaaaaaa aaaaaaaaaa aaaagaattg gaagcaacct aactgtccat |
| 81181 | caacagatga atgaataaag aaaaggtgac atatacacaa tggggtacta ttcagccata |
| 81241 | aaaaagaaca agatccagtc atttgtaaca acatggatgg aactggagat catcatgtta |
| 81301 | tgtgaaataa gccaggcaca gaagacaaac atcgcatgtt ctcgattatt tgtgggatct |
| 81361 | aaaaatcaaa actattgaac tcatggatat agagagtaga aggatggtta ctagaggttg |
| 81421 | gaaatagtag tagggagttg gagggaaggt ggggatggtt aaagggtaca aaaaatagaa |
| 81481 | taaataagac ctgctattcg atggcacaat agtgtgacta tagtcaataa taacttaatt |
| 81541 | gtacatttca aaataaccta atgagtataa atgtattgtt tgtaactcaa aggataaatg |
| 81601 | cttgagggca tggataccct attctccatg atgtgtttgt ttatctttgc atgcctgtat |
| 81661 | gaaaacatct catgtacctc ataaatatat atacctacta tttatccaaa aaattaaaaa |
| 81721 | ttaaaaagtt atactttgct tactttgctt atattggagt caattatact acatgattat |
| 81781 | tatgaatgat ataatcaaaa ttaaaagggt aaagttgatg cacaatttct ttcataatta |
| 81841 | gaagtgtcaa aagatgtaaa taacatagag aagtcagttc aaacaacaaa aatatcagta |
| 81901 | acaattttat catcctgaga gaatcttcaa agaaataaaa ttatgtgtca agttatttca |
| 81961 | taaaaagtgc aaatattctg ttctttatat cacaatgaag aattagcaaa taattcactt |
| 82021 | tgacttggta gatgtttaat aaataacata aaaaatcaaa tgtcatttga tccctgaatt |
| 82081 | ccatttgaaa agttatcaat ctctttaata aatagaattc aatttcaaat gttatcaatg |
| 82141 | ttagagtgta tacaatatcc tgcaataatt gattttccat tgaaaaataa gaattgacta |
| 82201 | agaagaaaac agtttaacag caatttctgc caactgaata gaataaaact taactatgtt |
| 82261 | ccctccagct ttctaatatt catgtagaga aattctttct ctaattgtaa ataggattac |
| 82321 | aactgtaaaa gtaatggcaa tatagttcta tatgaattat agagggtttt catatgtata |
| 82381 | ccatctgatt ggtggttatt tcagttaaaa ggagtaaaat ttaacccatt tcacttgtgg |
| 82441 | taataccttc ggtattaata aggagcacca atacaggact cacacaaaaa agttattttg |
| 82501 | cccatttttt ttctcatttt atcttcacag caatccagta aaatagatag ggtaagcaaa |
| 82561 | tccccatttt cacaataaaa cccccatagt tttagaaacc acatttggtg gcagtttgaa |
| 82621 | aagccaggtc tcctcattcc tcatctaagt tattttggta ttataaaggt ttttttaatt |
| 82681 | tttaagttat acagaatatc taattataaa cttctggcta ttttatagct aatatcttag |
| 82741 | atgtaagtgc ataatcagta taaataaata tattgaatta caataaaaca tcacatcaag |
| 82801 | tccacaaata tttagtcagt aagtgccaaa gagaatacta aattctgggc cacttatatc |
| 82861 | aattagttat aatttctgac atgattactc ctactatgga gtaggaacat catgaacaaa |
| 82921 | tgctcacaga aggcaaataa catcatggat ctgggatcag agtggggctg gtgggtgtat |
| 82981 | agatgggagg tgagaatgga taaagatgag tttgtatagc tggaatgtgt tgtggaaggc |
| 83041 | ttacagctaa aattttattt ggacttaatt aattttctaa tattaagaca taaaaggaaa |
| 83101 | ggtttataat aattagatca ttattttaaa aacatcttgc catttggaga ggatgtatgc |
| 83161 | atttctcctc caacttcagc aggttagttt cagtttcttt tgtgttttct ctgtttcttg |
| 83221 | tacacacttc tgttatcctc aaaacgttat actttaattt attcagttat gtaattgcct |
| 83281 | ctattttgac aatggagaca ctagtgaatg acaatttgct gaatgaatga atggataaaa |
| 83341 | tcagagagac tggaagcccg gagaatggtt aagatttaaa aaaaataaga caggaattca |
| 83401 | aaattgcaat tgattattaa ttcttacatg aaataaaatt ttcagaaaat attgtcatga |
| 83461 | tgaaaaaata ggactgctga gaatcagttc ttggaatcaa tatagtactt aaattcattt |
| 83521 | tttccacagt ttattaatgc aaactgtcta agcactctaa aRtattgaga gattttattc |
| 83581 | acctttgttc tcataaataa agtgatattg atacagccat aattttctta aataaagaag |
| 83641 | tattacagaa tgattttcct tcataaacat taaaactgtg gctcttggct gccttttgtc |
| 83701 | ctactgagat gcaaattaca aatgatagaa ataaaaaaaa aaattgaact gccacctgta |
| 83761 | gaaccacaca aatgccgggc atggcggctt acacctggag tcccagcact ttgggaggcc |
| 83821 | aaggtgggca gatcacctga gatcaggagt tcgagaccag cctgaccaac atggtgaaac |
| 83881 | cccgcatcta ctaaaaatac aaaaagtagc caggcatggt ggccagcgac tgtaatccca |
| 83941 | gctactcagg aggctgaggc aggagaatca cYtgaaccag ggaggcagac agtgcagtga |
| 84001 | gctgagatcg cgccattgca ctccagcctg ggcgacagag caagatgccg tctcagaaaa |
| 84061 | aacaaacaaa caaacaaaca aaaaacMcac aaatatacat ttatatggta tatttcctac |
| 84121 | taaaatatat ggatttattg tgttaattta aaataagatg caagacttag atacaaaata |
| 84181 | ctctgttggc ttgctgtatt atgcagggat ttaagttttt aaaaaatggt gaagggattt |
| 84241 | tggcttataa atatgaatac ctaataaatg aatgaacata ttgagatcat tttcattaac |
| 84301 | taaattaaat acacatattg tgttgataca tatgtatatc tattatacat atatataact |
| 84361 | atttcttaac acctccctgt tcccaccttg gtcttaactt acataggttc ttaccaactc |
| 84421 | tccttgcttc tactctttac ctctttaaaa ctattcttaa tacagcagta agagtgatcc |
| 84481 | tatttaaacg tcattcagct tatgtcaggt caattgcgtc tcatctcaca taagtacacg |
| 84541 | ttaaagtcct tacaatggcc tgcaagattt gacccctgct acttttggta tccgctcttt |
| 84601 | cacgtgttca ctcagtttca gccacgttgg cttcctttct gtcttgctgt tcttggcaca |
| 84661 | caacaagcat gctcttgctc aggatatttg tgtccttgtc acttctctgc ctataatgca |
| 84721 | agacaactcc cagatcattc tttcccctcc ttggtggctt tactgaattt ttgcctactt |
| 84781 | gccaaagctt tctcttacca cctcacctca aattgcaacc acatcctcct acgtgccata |
| 84841 | cccaccctcc ttgctatatt tttctcctta gcgtttatta tcgtctaata tatgatactt |
| 84901 | aatatattat atgagatata tttaacattt atatatgata ttaatatttg ttataatctg |
| 84961 | tcttagctat tagaatgtaa gacccatgct ttcttcactg aagccctcac atgtccaaga |
| 85021 | agagaaatta ctttatacaa tatgtactaa aaaaaacttt gaaaaaatga atgaataaat |
| 85081 | aatggcattc agcaaactca agtgctgtgg ctgacagtga ctacgatttg gggaatgagt |
| 85141 | agagtgtaaa aggaaacaaa agtagcttaa gagaaaaata aaggaatctt attttccttc |
| 85201 | actactctca atgaaatttt atacacacac acacacacac acacacacac acacacacac |
| 85261 | acaccccttt aatatgaatt tcttgaggac tgaagtaaaa acctgcattc ttgtaggcta |
| 85321 | ttgtttccac ttcggcactt atcattgtat ctggaatagt actccataac tatgctgaac |
| 85381 | tattaaaaat taattaaaat aaatgagaac cttaaaagat ctgtatagtt cctggacagt |
| 85441 | gtcttgccct caggagctta tagtctgctc ttagaaacta cctgaaggtt agttggaatt |
| 85501 | ctttcgagtg ggggaaaaat ctgtaagctc catttagtca tggtgttggt tctaatgcaa |
| 85561 | gcagagtcaa ccaatgtatt ctgcatttct ttatttctgt attttctttc aatgaaacta |
| 85621 | aaagaataca tatatcaaaa ttaaaaggaa gacaaacatc attaatttta aaaaaagagt |
| 85681 | tttggtccag gtgcagcggc tcaggccttt aatcccagaa ctttgggagg ctgatgcagg |
| 85741 | aggatcgttt gagcacagaa ggtcaaggct agcctgggca acatggcaac actcctgtct |
| 85801 | ctacaaaaaa tttaaaagaa ttagcatatg tggtagtgca tgccttttta ctacttacaa |
| 85861 | tattctaagc atgacattat gtttcctata tcttattcct aaatttgata acaattttgc |
| 85921 | caactattat cccWgttatc cagctaagaa cacctgggta ctaaaagttt taaaaaatcc |
| 85981 | aggcaataag taccagatct gaatttaaat cctgatctaa ctctaaaagc atggctgtct |
| 86041 | acaaaccact ggagatacta aagcattggc tggtggaaga tctggtatgg atattaaaaa |
| 86101 | gagaggttag catccttgct tccaaagtaa ccactccaag cttacgaaaa agtgcagtct |
| 86161 | attccaagaa atattggtta gacaaatccg gaaatctaac tgtaggaaca attgttcctt |
| 86221 | atgaagcaat atcactgaac acagaaatac ctcaaagtat gtcaaccttg atgaaggcaa |
| 86281 | tcaaattagg atttggggca ttgtaccaca taaaacagga cattatttag tttccttcat |
| 86341 | gtccctatca caaagattat aatgtaagca taattctcct ctgataaatt tcaaaataaa |
| 86401 | tggataaaga agagaaatat gtttctttgt actggcaatg tatctaatca ctagtatcgt |
| 86461 | gatgtggtgt gatatcacta catcttgaaa ataatatatt agaacctatc agcccaggga |
| 86521 | ccagtaattt gtatttttct tcacagaaga gagtttccta aatatatatt agaaaatctt |
| 86581 | catatcaaca tgaWatgtgg tctgtgtcat gtgtacctaa cgcaccagct ttaaggaatt |
| 86641 | ttgagaaatc tcatgctaat gaccaatccg tacctggtaa tccccctgga acaagaacaa |
| 86701 | ggaagctcac tcatctgtgt gctgtgtttc ttgatctcca accttgatca ctgccatgcc |
| 86761 | atctgtaaca tatgccaaaa aagcataagc agaaatagag actttcttgc ctaggagcat |
| 86821 | tcttccttat gttatgctgg agtgcgtcct ctgtcctcta gtaatgctac tggaattagc |
| 86881 | tcttctacca aagacttttt catgacaagg aattccagag gccaacttct aaaatcaaaa |
| 86941 | ccaccactcc aaaaacagct aatttcatcc ttacttcaat ccctgattct cagcattaca |
| 87001 | tccaacagca ttttcttgcc agaaaccctg tcagttcatc tttaagaaga agagcaagct |
| 87061 | agctggacca atttcactca cctactctga aacaactaaa aactaagaat ataaaagtat |
| 87121 | gcatgaacta taattcatgt cacagaggca tttcctcaca gaatacctat ggacaaacaa |
| 87181 | tgaagtttct ctcatttcat tacaagtctt atggccttgg acgtgcttac ttgccatgta |
| 87241 | gctctggcta aatctataag caaacatagg atatcactgg ttttagcttt gtgctcttcc |
| 87301 | atgctgtgtt accactgatt cattaagaat gttcttgaag acacattcag attttaatgt |
| 87361 | tatatttcta tcttagctcc aaagagtttt acaaaaattt tctaaattta tcaattataa |
| 87421 | taatatagat acaagttatc ttcaaaagat tttattaaat taacaaagtt gaacaactta |
| 87481 | tcaatataaa caattgtttt aggttcattg gatatatgat atttagtcct tgtggattca |
| 87541 | ttgttctctt acaaaaattg ctgaagtttt aaaagtatca cgatggtaat gtatttcaca |
| 87601 | attatgttat aacacagaca actaggatga aaattaattt ggagaactag ttcaaaaaat |
| 87661 | ccagtgatta aataaagctt agaaccaatg atatgtatat aaaagtttag atttagtcca |
| 87721 | tgatattaac caagtaaaat tgattttatc tatattttct actctacctt ttctggagat |
| 87781 | ataatattgg cataaattga taaaaccata taatattgtt aataagttta acaattgcta |
| 87841 | tggataattg ttttaatgtc ctaactcttg ttattgtatt aattcagaat acaatgacta |
| 87901 | agtctctaag tagagcaaaa acctgcattg tgaacttatt gaagactcaa ctggacatga |
| 87961 | ttatatgtct aatttttttc caaattaatc agtctcttca atgaaattac tatcaaatct |
| 88021 | caacagagat tgtcatgaaa attgaaaaac agattccaga atttatatgc aaaaccaaag |
| 88081 | ggtcaagaat tgttccgaga gcatcctgac aaacggctct aggatggatc cacctacgaa |
| 88141 | atgccaggat ttaatataaa gatgttacaa ttaaaacagt gtgatatcgg tgtagggata |
| 88201 | gaaaaataga ccagaacaga gaacctggaa aagatctgtg catgcctttt aaaacttaat |
| 88261 | atatgacaga tgggactagc agatcagtgt gagtaataat acactattca aaaaatggtg |
| 88321 | cacagtagta gtaaagaaaa ataagaaagc aatcctattt aaaacagcta cagtataatg |
| 88381 | aaatacctaa gaataaattt aactaaataa atgaaagatc tttacaatgg aaactataaa |
| 88441 | acatgaaata tcttttcatt cttatgtgtc ctcttcaatt ttttcaatgg ataaaaaata |
| 88501 | gaaaggacac ataaaaaatg gaaagatatt tcatgttcat ggattgaaaa aattaatatt |
| 88561 | gttaaatgtc cacactatgc aaaatgatct gcagattcaa tgcaatctct atcaaaatat |
| 88621 | caatcaatgt cattcttcac agaaatacaa aaaaatttct aatagttgag tggaaccaca |
| 88681 | aaaggccctg aatagacaaa gcaatcttaa gtgaaaagaa caaagctgga ggcatcacac |
| 88741 | tgcctggctt caaaatatac tacaatggta tactaaccaa aagagcatgg catggcataa |
| 88801 | aaatagacac atagactaat ggaacagaat acagaaccca gaaataagta catgcattta |
| 88861 | cagccaacac agtttcaaca aaggcacaaa gaatatacgc tggcaaaagg atagtctttt |
| 88921 | taagaaatgg tgctgacaaa actggatatt catatgcaga agattaaaat tagagcatta |
| 88981 | tctcttacat atcaaaataa actctaaatg aactaaagac ttaaatttaa gacctgagct |
| 89041 | tatgaaacta ctagaagaaa aacattaaaa gaatactcca ggacattggt atgggcaaaa |
| 89101 | atgttttgag taagacctca aaagcacaag caacaaaagc aaaatttgac aaatgggatt |
| 89161 | acgtcaacct aaaaagcttc tgcatggcaa aggaaacaat caacaaagtg aagagacaac |
| 89221 | ctacagaatg ggagaaaata tttgcaaact atccatctga caagggatga ataggtagaa |
| 89281 | tgtataagga actcaaacaa cttaatagca aaaaaaaccc caaataattt gatttaaaaa |
| 89341 | atgagcaaaa gatctgaata tacatttctc aaaagaagac atacaaatgg ccaacagata |
| 89401 | cattaaaatg ctcaatatca ctagtcatca gggaaatcca aattaaaacc acaRtgagat |
| 89461 | ttcacctcac cccattaaaa atggctcttt tcaaaataga gatgctggca aggagatgag |
| 89521 | aatagcagat gctggcaaag acttgaagaa agagaaatgc tcatacactg tttgtggcca |
| 89581 | tgtaaattag tccaaccact ataaaaaaca gtatggagat tcctcaaaaa accaaaaata |
| 89641 | gaactaccat acaatacact gatcccactg ctaggtatat atcaaaagaa aggaaataag |

Following is a REPS2 genomic nucleotide sequence (SEQ ID NO: 7).

| >X:16315151-16528450 | |
|---|---|
| 1 | ctctgtcacc caggctggag tgcagtggtg ccatctcagc tcactgtagc cttgacctcc |
| 61 | caggctcaag tgatcttccc acctcagcct cccaagtagt tgggactaca ggagcacacc |
| 121 | accatgcctc gctaattttt gtagttttta taaagatgag aactcgctat gttgcccagg |
| 181 | ctggtctcga actcctgggc tcaagcaatc catcggcctt ggYctcccaa agtgctggga |
| 241 | ttacaggtca ccatgccagg ctgagacagt aacttgtaag atttaattgt tggtcgggtg |
| 301 | cagtggctca cccatgtaat cccagcactt tgggaggctg aggcaggtgg atcatgaggt |
| 361 | caggagttca agaccagtct gaccaacatg gtgaaactct gtctctatca aaaatacaaa |
| 421 | aattagctgg gcgctgtggc gggtgcctgt aatctcagct actcgggagg ctgaggcagg |
| 481 | agaattgctt gaacctgggt ggcagaggtt gcagtgagcc gagatcatgc cacggcactc |
| 541 | cagcctgggt gacagagtga gactctgtct caaaaaacaa acaaaaaaag atttaattgt |
| 601 | cattagtggt ttgtggggca cataagggtt caagaacttt aacccctgtc tttcttgatg |
| 661 | agtttagaac tacctgggta aggtccatgt gcacatcaac cttgttgttg ctagacttgg |
| 721 | gttggagatg atgattagga ttgcagccac actcaggcta cttcttacat attctatccc |
| 781 | cttacttccY ccacctcaga gtctataaaa cagacctata cctgggaagc tgcWaggaaa |
| 841 | ttgctgtctt ctaatcatca agtcctgcag gtggtaacag agaggaccca aattacgtgt |
| 901 | gctttctaga tggaatcggg gttagagtgg gggtagagtg gagtcccctc tgagggcaac |
| 961 | agaattcaga ggtggtagta ggagagggag gtaggagaga gagaaagaaa gagacagaga |
| 1021 | gagagagaga aagaatatta gcctgttctc acactgctaa taaagacata cctgagactg |
| 1081 | gataattcat aaaggaaaga ggtttaattg actcacagtt tcacatggct ggggaggcct |
| 1141 | cacaatcatg gcagaagttg aaggagRagc ataggcacgt cttacgtggc agcaggcaag |
| 1201 | agagcttgtg taggggaact gctctttata aaaccatcag atctcatgag acttaatcac |
| 1261 | tatcatgaga agagcatggg aaagactcac ccccataatt caattacccc ccaccaggtc |
| 1321 | cctcccatga cacgtgggaa ttatgggagc tagaattcaa gatgagattt gRgtggggac |
| 1381 | acagacaaac catatcatgg aggaaaagag agggggagag agacagagag agagagaatg |
| 1441 | aaccaagaat gatcaatttc ttctcccttt catcacttct ctttccttag agaagagtga |
| 1501 | gggggactag gagaagtgcc aatgttactt catcaatatt ccttcctgag gagtagagag |
| 1561 | gaagctttct catgaaaata atgaggccag gggaatagga ttctctctca cactgttaaa |
| 1621 | gatctgcctt ttttcactaa tagcagaaaa aaatattaaa atcattcaaa caggtgatta |
| 1681 | tttagtgttt atattggtcc atcccttaat aaataccttt caaaattcac caatctttga |
| 1741 | gatacactca atagtacagt catgcgttac ccaatgacgg gtatgttctg agaaacacgt |
| 1801 | tgttgggtgt cttcgttgtt gtgtgaacat cacagggtgt acttacacaa atctagatgg |
| 1861 | tacagcctac tacacaccta ggctatgtga aaattgtcag aatcaaaatg gagtcgcttg |
| 1921 | tgttaaaaaa aaacacacaa aaacctgaca aatagagctg aggaaggcta cgaagagagg |
| 1981 | gttctcacac ttgtatgcct gataacaaaa ttgcaacaaa agactgcaaa aactgcaaac |
| 2041 | ttacacaaat gtcatcacaa ccttctgtaa agacatctga ccagcaactg cctgtctaaa |
| 2101 | cttgaactgg tgccatcctt gttattgatc cttgtcacca aaaataacca tttcaaaaca |
| 2161 | actatgtaac cttcattttt ccttgaaaaa tttttgtctt cctttacctc cacgaatatg |
| 2221 | cacaagttta ctgtggcata catattccca ttgcaatgcc ctattcccac atgaacaaca |
| 2281 | ttttcttttc ttttttcttc ttcttttttt tttttcttta gacagagtct ccctctgttg |
| 2341 | ctcaggctga gtgcagtggt acaatcacga cttactgcag cctcagcatc ctgggctcaa |
| 2401 | gtgatcctcc cacctcaggt tcccaagtag ctaggactac aggtgcaYga cacaatgccc |
| 2461 | cacacccagc taatttttgt atttttttgt agagacaggg ctttgccatg ctgcccaggc |
| 2521 | tggtcgtcaa ctcctgggct caaatgatcc acccacgttg gcctcccaaa gcactagcat |
| 2581 | tgcaggcatg agccaccgtg cctggccata aacatcattt tcttttagag agcctctctc |
| 2641 | tgacatttaa gttgacagct atatggtaca gcctattgat cctagactac aaacctgcaa |
| 2701 | gcacagcatg attgtattag tcaattctca cactgctgta aagaaatgcc tgagactggg |
| 2761 | taatttataa agaaaagagg tttaataggc tcatggtttt gcaggctgta cgggcagcat |
| 2821 | ctcttctggg aaggcctcat ggagctttta ctcattgtgg aagacaaagt gggagcaggc |
| 2881 | atcttacatg gcagaagcag gactgagagg ggttgtgggg aggtgccaca cacttttaaa |
| 2941 | ccaccagatc tcatgagaac tcactcacca tcgtgagaac agcaacgagt ggaaggggct |
| 3001 | aaaccattca tgaaggatcc acccccatga tccaatcatc tcccaccagg ccccacttcc |
| 3061 | aacactgagg attataattg agcatgagat ttggatgggg acacagagec aaaccatatc |
| 3121 | attttgcccc tagcacatcc taaatctgat gtccttctca ccttgcaaaa tcccatcatg |
| 3181 | ccttctgaat agtccctgaa aatcttaatt cattccagca ttaactcaaa agtccaatgt |
| 3241 | tcaaagtctc atttgaggca aggctagtcc cttctaccta tgagcctgta aaataaaaaa |
| 3301 | caagttagtt acttctgaga cacaatggag agataggtat tgggaaaata tacccattca |
| 3361 | aaaaaaaaga aattggacaa aagaaagggg ctacaggcct catgcaagtt tgaaacccag |
| 3421 | cagggcagtc attaaatctt aaagctccaa actaatctcc tttgacacca cgtcttgtat |
| 3481 | ccaggacaca ctggtgcaag aggtgggctc ctaaggcttt gggtagctcc acccctgtgg |
| 3541 | ctttgcaggg ttcagccccc atgacagctc tcatgggctg gcattgagtg cttgtgcctc |
| 3601 | ttccaggtgc agggtgcaag ctgttggtgg atctaccatt ctggggtctg gaggatggtg |
| 3661 | accctcttct tacagctcca ctaagcagtg ccccagtggg gattctgtgt gggggcttca |
| 3721 | accccacatt tcccactctg cactgcccta gtagaggttc tccatgaggg ctctgcctct |
| 3781 | gcagcagtct tctgcctgga atccaggctt ttccatacat cctctgaaat ctaggtggag |
| 3841 | gctcccaagc ctcaaccctt gcactctgca cacctgtagg cttaacacga ggtggaagca |
| 3901 | gccaaggctt gtggcttcta ccctctgaag cagtggtctg agctgtacct gggccccttt |
| 3961 | gagccagggc tggagcaaga gcagcttgga tgcaggaagc aatggcccaa gggtgcacag |
| 4021 | ggtggcaggg ccctaggcct ggctcaggaa acctttcttc cttcctaggc ctctgggtct |
| 4081 | gtgatgggag aggctgcaat gagggtctct gaaattcctt tgaggccttc tccccattgt |
| 4141 | cttaggtgtt agcacttggc tcctttttac gtatgcaaat ttctgcagcc tgctttaatt |
| 4201 | cctcccctga aaatgggctt ttcttttcta ccacatggcc aggctgcaaa tttttcaaac |
| 4261 | ttttacactc cgcttccctt ttaaatgtaa gttccagttt caggtcattt ctttgctcac |
| 4321 | atatatgaac ataggctgtt agaagcagcc aggccatatc ttgaacctta gaaatgtctt |
| 4381 | ctgccagata ccctaaatca tctctctcaa gttcaaagtt ccacagatcc ctagggcagg |
| 4441 | ggcacaatac aacaaagctc tttgccaaaa tgtaacaaga gtagcctttg ctccagttcc |
| 4501 | cagtaagttc cttatttcca tctgagacct catcagcctg gccttcattg tccatatcac |
| 4561 | tattagcatt ttagtcataa ccattcagtc agtctctagg aagttccaaa ctttcccaca |
| 4621 | tcttcctgtc ttcttctgag ccctccacac ttttccaatc tctgcacgtt acccagttcc |
| 4681 | aaggtcactt tcacattttc aggtatcttt atagcaatgc tccactccta agtaccaatt |
| 4741 | ttctggaaaa aaaaaaaaaa accaaacaaa aaaaacataa tgtaaagaat atctctgcaa |
| 4801 | tgggaataat tggattccat gagaaacaat aaaaaagaat atctcttgat gtctcttggc |
| 4861 | tatttctgaa aatatgatcc ttctggcttt tctggccatc atggagccaa aacaagtgtg |
| 4921 | taagctttga accacaaaaa agttcttgtt tactgtcagc cttaagggat atgagtgctt |
| 4981 | ctagtttcta aattcttatc tgtgtttatt ttgaagtttg ataactattt cttctattga |
| 5041 | aaaaactttt agaaataaag aacagtactt ggcattcctt agatattaac atatttttac |
| 5101 | aagtgccatt tttttttcct cctgtgccct gggtattact gctgttgggt cccctttggc |
| 5161 | tgtgccccac atcttttgtt gagcagcttc tttcttacat cctggagacc ttcagagctt |
| 5221 | ccctttcttc tgacccagcc tgctgtccct cactcattat ttccaggttg ccaagtctct |
| 5281 | ggggctcctc Yctttctgtc gtgtcctttt acctccccag ttccacccac tctcagtcaa |
| 5341 | cagccatctc agccccactg atcaagcctt cctggctgag ctctggctca tgaattttct |
| 5401 | ctcttgtttc aactcccatg gcagttaacc cagagtatac agtttagcac ttaattgtat |
| 5461 | attttctttc atttattttt atttgttgtg ttagttttgt ctcttccagt gaggcttcaa |
| 5521 | actccttgaa agcaacacat cttctattaa agaaaaattc ctcttagtac tctggtaggg |
| 5581 | atacagtaaa tactctcttt tctatcaata aatgtcaatt gtctcactga tagattgata |
| 5641 | ggcacagatg tgagactttc tacctgtctc cgtcatatct ggtgcttaat aaatatctgt |
| 5701 | caaatttatt atacttagtt gagtcataaa gatcagttct gaatctggta ttcagtggga |
| 5761 | caaatcacac tgtctctgca tccattttct catctgcaaa atggagatta aaaatgtctt |
| 5821 | cccaatctac ctcagagagt tactatgagg cttaaatgag ataatagtta caaaagtgct |
| 5881 | ttgaaaagct ttgcagcttt tttcctctac tgcatgaaga ttagggatta ttatagctca |
| 5941 | catttccttt gacagtagtc acagggtaaa gtagggtttg gtgtttatca cacagagtgt |
| 6001 | gtccctttct agcctttatt agaatggttg gccattctca gggaaacctg agtacaacca |
| 6061 | gaaaggtagg aagagaataa acatctggct tccagttttc ctgagaaggt atcacagcta |
| 6121 | tggaaactac aaaggtggtg aaatttcttt tcttttcttc ttcttcttct ttttttttaa |
| 6181 | gacagtttcg ctctgttatc caggcgggag tgcagtggca cgaactcagc tcactgcaac |
| 6241 | ttccgcctcc caggtccaag agattcttgt gcctcagcct cccgagtagc tgggattaca |
| 6301 | ggtatgagcc atcacgccca gctaattttt gtatttttag tagagacagg gttttgccat |
| 6361 | gttggccagg ctggtcttaa actcttggcc tcaagtgatc ttcccatctt ggcctcccaa |
| 6421 | agttctggga ttacaggcat gagccaccat gcgtggcctt ctttcctttt ttaaattctt |
| 6481 | tttttctttt taaattttaa atagaagcag ggtctcatta tgttgctcag gctggtccgg |
| 6541 | aactcctggg ctcaagtgat cctcctgcct cagcctccca aagtgatagg attgcatgca |
| 6601 | tgagctacca cgcctggcca ggtggtgaga ttttgaaatg gaaattccca agtgaagtta |
| 6661 | gtctttattg ctcctaattt acatatcctt tctgcactct tgttccattc cgaagctttc |
| 6721 | tgttgtttta gttccctcct ggcaggttgg atgcaagacg tggtgtaaag aaagcttagt |
| 6781 | gaagaggtga gaacttcttc cttctggaga agatcctctt ctctgttcca tttttccaga |
| 6841 | ttccaaaagg aaaatattac tctggttatc actgaggttg acatcatttt ttatttttct |
| 6901 | tcttcttatg tatctctctt cactctctcc ccctttcctt tccttctttt tgtatttaca |
| 6961 | atttcatgat tttttcccct tctcctctct tcctgacctt ggtcctttag ttgaccataa |
| 7021 | taccaaagta tatttgttat cgtcagtggt atttcttgag atgttttttg tctctgtttt |
| 7081 | taataaaaat aaaactattt ggggtgtaag ttttatatat ccctttatta aagcaaatta |
| 7141 | taaaatgtat taattttttt attgatacat aataattgta catatttacg gggtatatgt |
| 7201 | gatattttgg tacatgtata caatgtgtaa tgatcaaacc agaataactg gcatatccat |
| 7261 | aatctcaaac atttattatt tctttgtgct cggaacattt caaatcttct aggtattttg |
| 7321 | aaatgtacaa taggctgggc atggtggctc atgcctgtaa tcctagcact ttaggacgct |
| 7381 | gaggcaggtg gatcacttga gccctaggag tgggctagcc tgggcaacat agcgagacct |
| 7441 | tgtctctata aaaaatacac aaattagctg ggcatggtgg tgcacgccta tagtcctagc |
| 7501 | tactcaggag gctgaggtag gagaattgct ggagcccagg aggtcgaggc tgcagtgagc |
| 7561 | cgtgattgca ccactgcact ccagcctggg tgacagattg agaccatgtc tcaagaaaaa |
| 7621 | agaaatacac aataaactgt taattatact caccttactt tgctatcaaa cactagattt |
| 7681 | tactccttct aactgtattt ttgtatccat tagctaacct cccttcgtcc cccagcccac |
| 7741 | ctttcccagg ttctgataac tattgctcta cctccatgag atcaactttt tcagctctcc |
| 7801 | catatgagtg agaacatgtg atatttgtct ttctgtgtct ggcttatttc acttaacaca |
| 7861 | atgaccttca ggtccatcca cgttgctgca aatgacagga tttcattctt ttttatggct |
| 7921 | gaatagtatt ccattgtgta tatataacac attttcttta tccatgcact attgagggac |
| 7981 | acttaggttg atccatatct tggctattgt gaatagtgct gcaataaaca tggaggtgca |
| 8041 | ggtatctctt tgattactga tttactttcc tttgaataaa tgcccagtag tgggattgct |
| 8101 | ggatcacata gtagttttgt ttttagtttt ttgagaaacg tccatactgt tttctataag |
| 8161 | ggcagtacta atcgtattct caacagtgta aaagactttc cttttctctt atatatggag |
| 8221 | tgagatgata tctcactgta gttttgattt gcattttcct gatgataaat gatgttgagc |
| 8281 | atgttttcat atacctgttg gccatctgta tgtattcttt tgagaaatgt ttatgcagat |
| 8341 | cctttgccca ctttttaatg ggattattat tatttttttg ctgttgagtt gagttccttc |
| 8401 | tatatgctgg atatcagtct cttgttggat gaatagtttg caaatatttt ctcccattct |
| 8461 | tcaggttatc tcttcatact gttaatgttt cctttggtgt gcagaagcct tttagtttaa |
| 8521 | tatatttcca tttgcttatt tttgtttttt tggcctgtgc tttcaaggtc ttagccataa |
| 8581 | aatccttgtc cagaccaatg tcctgaagca tttccccaat gttttcttcc agtagtttta |
| 8641 | cagttttagg tcttatgatt aagtctttaa tacatcttca gttgattttt gtataggtga |
| 8701 | gagagacata aaggtctagt tttattcttc tgcatatgga tatctggttt tcccagcacc |
| 8761 | atttattgga aagactgtcc tttcccaatg tatttccttg gtggtttgtt ggaagtcagt |
| 8821 | tggctgtaaa tgtgtgggtt tatttctggg ttctccagtc tgttctattg gtttatgtgt |
| 8881 | ctcttttcat gtcagtacca tgctgttttg gttactatag ctttgtagta tattttgaag |
| 8941 | tcaggtagtg tgatgcctcc agctttgttc tttttgctca ggattgcttt ggctattcag |
| 9001 | ggtcttttgt ggttttatac aatttttagg attgtttttt ctatttttgt gaacaatgtc |
| 9061 | attggtattt tgatagggat cacagtgaat ctgtagattt ctttgggtag tatggtcatt |
| 9121 | ttaataatat taattcttcc aatccatgag catgggatgc ctttccatgt ttttgtgtcc |
| 9181 | tcttcaattt tttaaatcaa tgttttgtag ttttctttgt agtaaaatat attttttatt |
| 9241 | agtgcttaac cctagactga tgttttcttt tgggttattg tttcccagtg aggcttcatg |
| 9301 | catatatttc tgcaggtctt cccaggaaaa atgacattta aaaagagaat ttttggaaaa |
| 9361 | gagatgcaga tatgtgggcc tgatgctgga acttccagtg caatggtggg acagactttt |
| 9421 | tttccagtgg cttcttttct ggtctctctt tctctctctc tctttgagac agagtcttgc |
| 9481 | tctgttgccc aggctggagt gcagtggtgt gatcttggct cactgcaacc tctgtctctt |
| 9541 | gggttcaagt gattctcctg cctcagcctc ccaagtagct gggactgcag gcactcacca |
| 9601 | ccatggccca gctagttttt gtatttttag tagagatggg atttcaccac gttggccagg |
| 9661 | ctggtctcga actecaggac tcaagtgatc tgcctgcctt ggcctcccaa agtgctggga |
| 9721 | ttacaggcat gacccaccgc gcccggcctg gactttcttt taaaagcggc tagagggcca |
| 9781 | ggcgtggtgg ctcaagcctg taatcccagc actttgggag gccgaggtgg gggggtgggg |
| 9841 | gtggggtgga tcacctgagg tcaggagttc gagaccagcc tgacaaacat ggtgaaaccc |
| 9901 | cgtctctact aaaaatacaa aaattagcca ggagtggtgg caggcgcctg taattccagc |
| 9961 | tactcgggag gctgaggcag gataatcgct cgaacccggg aggtggaggt tgcagtgagc |
| 10021 | caagactgtg ccattgaact acagcctggg caacaagagc gaaactcctt ctcaaaaaaa |
| 10081 | aaaaaaaaaa aaaaaagtgg ttagagtact agccaggggg aagcaagcac atttgaaaaa |
| 10141 | tatagtgtgt atgctttagg atagggagga atggagagag agaggaggac atggaccggg |
| 10201 | gtggaaaaat gtggggtttc gcattgattt ttggtgtgag cttgaacaag ttttgtacta |
| 10261 | aaaaaaatgc ctgttttact actaaaataa gtgcttcctt tggagagtga ggatcctctg |
| 10321 | ttactgaggt ttgtattttc aggccctagc atataaatct gctacatacg agatattcaa |
| 10381 | ctggttttgg aggaatgaag gtgaacatgg cagtttcctc atctgtaaaa gcagggtttg |
| 10441 | gacgaaatga tctgcaaaat gggtcttttt gtactcttac tactctagta tttacgagta |
| 10501 | accgatgtta ttccttataa agtctcaact cctggtKtga gccatgtctg acgagggggc |
| 10561 | ctaactccaa taccaccctg aacactctgg ggtcagtggg accctgtttg tacagacgtc |
| 10621 | tgcgtcttcc ggctctttag gacataagca gtactgctat ggaagaaagg aaacctaatt |
| 10681 | tcccatgcct tgtccctttc tggaaagggc atggcgtggt gtgggcacca cacctgcccc |
| 10741 | agccttctag caagaaaaca acacaacctt ccccgctccc cgaaaagagc ttctctccag |
| 10801 | ttgggtctag atgccccctt cccaaccagg gcggatggcc aaaggctccg ctcccttgca |
| 10861 | gggtaggctt ggaaaggcac ggggcccgcc ttcctgggca ccggagcctg tgacgggcgg |
| 10921 | agggcgtgcg gcgcgctggg ttagttacct ggcgctgggc ggggcaagcg ggaggcgggg |
| 10981 | gcggggccgg ggtgacatct caggctgtga ttgggccgcg ccgcgcctgt ctccatgtgc |
| 11041 | cgcgcagctg aggcacaaca ttcaagcccg ggggtggggc cggcgcgcgc cgggaggaag |
| 11101 | cggccgcgcg gcagctgcgg ggcgtggggg tggtggtggc ggcggcggtg gtggcggcgg |
| 11161 | cggcggcggc ggcagctgag gccgaggagg cggtggctgt ggcggacgca gcagcacccc |
| 11221 | agctagggac agggctccgc cgcgccccct tgctggcccc atggaggcgg cagcggcggc |
| 11281 | ggcggcggcg gcagcggcag cggcagcggc gggcgggggc tgtggctccg ggccgccgcc |
| 11341 | gctgctgctg agcgagggcg agcagcagtg ctactccgag ctcttcgcgc gctgtgccgg |
| 11401 | cgccgcgggc gggggccccg ggtctgggcc ccccgaggcc gccagagtcg cccccggcac |
| 11461 | ggccactgcg gccgccggcc ccgtggctga cctgtttcgg gcatcgcagc tgcccgccga |
| 11521 | gacgctgcac caggtgggtc cctccgcctc ctgtccctgc gggtctgtgg ggcagtgtgt |
| 11581 | gcgggggcgg aggttgcgag tggggcgaca gggctgcccc cagggacccg gaggcctcgg |
| 11641 | ccagttctga gagagaggac tctgcagccg gggatggcgg cgacgccctt gtccccgggg |
| 11701 | gacaccagcc tccttcgcag ctcagctcct cgcccagtgc tgggtgcgct ctccccatct |
| 11761 | tccttccctg agacagtctg acactctctc ccctcccccg gccatttctg gggttctgca |
| 11821 | gccgcccaga cccaaagttt gtcctgttct ggaagtggtg tttgttttgg ctgctgcctg |
| 11881 | gtttaacctc tgctcgctgc tcctgcccct ccctctcctg agtgggttct cagggtgcct |
| 11941 | tgctgggatt tcaacttgaa gggtgtgtgt gtccgtaggg ttcgaggccc agggctgaat |
| 12001 | gttgtcctct ctatgtagtt gtaatatata agtatctact atatgtaatt gcccataaca |
| 12061 | tatttctaaa tattaatatt aaatcgaaaa ccttaatgac aagttgcaga acagggacac |
| 12121 | gggactgcaa gaggacgttt gtcaagtcaa aaccaaattt cccatttgag attagaggtg |
| 12181 | gacttcagaa gtccaccatg ggaaacagct gtagtttccc atctatcatt acttggactt |
| 12241 | gaaatagaat tgaaaggtaa gggatgtcat agctaccacc ttttggctgt tctatacttg |
| 12301 | gaaaagtact aggggatgat acataccaga cgctgttttt gttttgtgct ttccttggag |
| 12361 | cttggagaaa gactacttga taagcctttg tctgtcattt catgtcacca gcacttcatg |
| 12421 | gtgataagac tgttaatatc aggttcaaaa cgtgaaatga agcttttcct aaaagcaata |
| 12481 | gtatgaatgt tattaacacc aatggaatat ttagagtggc gagtggtatt ttaaaatcta |
| 12541 | gtttcaaagg agaactgagg gttttatggt tcacttgcaa tgtcattgtg ttcatacgta |
| 12601 | tttttaaaaa aattgcttaa gactgcgttt tatactgctg gagtgttttt actgctatat |
| 12661 | gtttgtgagc ataactttag acactttgta atctttcacc cttttgaatt tgggatttct |
| 12721 | gaaactgtaa ctagtattca cagaacttag catggctgtt tatccctaaa gaagtccttg |
| 12781 | atatgtagac agttctgcca ttgctgatga gtagttgagc tggatcaggt aaagagaaag |
| 12841 | tcagtgtctt aaaacacaaa ccgtggctat tacatgaggc cctcagaata gtcccgagca |
| 12901 | taatacattt attcagtgct gtagctgctt cccagctgaa agaggaataa ttgactgagg |
| 12961 | ctatgcagca agtaagtgac agctgggtac ctattaaggc ttattttgtt gtcagtaatg |
| 13021 | catcggataa gaggaggtgg gcatagtaaa tctgaatatg ttggttgatg cctctttagg |
| 13081 | aattgggaag aaggatttcc tgatgttaga acccatgctt tcttgaggta catatagttt |
| 13141 | taaaaggacc caaggcacag tggctgtgcg tgaatgagat tatatttttg atgtccagtt |
| 13201 | gaaatctgct tgcaaaagac tgcttcagat actggcaaaa ggggacatcc ctgcgagatt |
| 13261 | gcttggagta aatctctcag agctcgaagc tcccttattc agcctagcaa agcaaaacac |
| 13321 | atcatccacc tttaatccaa caaaattacc ataactcaat attcttggta caggtgaaaa |
| 13381 | tgaacattga aggcatcctg gacatgtgat cctaagagag tcaaacaaag tcagcaacca |
| 13441 | gcagacacct aattttcacc catcactttg gtggtggtgg tggtggtggt ggtgNtggtg |
| 13501 | gtgtgtgtat gctccatcca ctttaatttg cattacccca tcagtgtatt agccattctt |
| 13561 | taaaacaaac gtagatgttt ttaccctttc tctgttaaac aggaacctgt aagttgagat |
| 13621 | ttaagtgccc caacaggtat aactgctcaa atcttggagg cactaaccag tcctgatatt |
| 13681 | ttctatggta acaaccaggt tttcctctta ataagcatac aattaagatt accataataa |
| 13741 | tgttccagga agagtgagaa tcttgcattc ttaccacaag taaatggtct ataaaaatac |
| 13801 | ttatttttta tttatttatt ttttgagatg gagtctcgtt ctgttgccca ggctggagtg |
| 13861 | Yggtggcgtg atcttggctc actgcaacct ctgcctcccg ggttcaagcg attcttccgc |
| 13921 | ctcagcctcc tgagtagctg ggactacagg cgcacgccac ctctcccggc taatttttgc |
| 13981 | atttttagta gagacggcgt tttgccatgt tggccaggct ggtctcgaac tcctgacttc |
| 14041 | aggtgatcca cccgcctcag cctcccaaag tgctgggatt acaggcgtga gccaccatgc |
| 14101 | ctggccttaa catatttttt aatagactat tttttttttt cagagcagtt ttaggttcac |
| 14161 | agcaaaattt agcagagtac ccatatatcc cctgccccga cacacccgta gctttcctta |
| 14221 | ctctcaacgt cccacatcag aataatagag tttttacaat tgatgaacct acattgacac |
| 14281 | ctcattattc cccaaagtSt atatattagg gctcactctt ggtgttgtac attctgtggg |
| 14341 | tttggacaaa tgtataatga cggttccacc attgtagtat catacagaat agtttcactg |
| 14401 | ctctaaaaat ctgtgctcta ttgattcatc cctccctctc cactaacatg tggcaatcac |
| 14461 | tgattttttt ttttttttta ctgtccccat agttttgcct tttttagaat gtcatatagt |
| 14521 | tggagtcata cagtatatag gcttttcact ttggcttctt tcatgtagta ctatgcactt |
| 14581 | aaaattcctt catgtctttt tatggtttgg tggctcattt ctttatagtg ctgaataata |
| 14641 | ttccattgtc tgcatgtact acaatttatt tattcaccta ctgaaggata tcttggttgc |
| 14701 | ttccaagttt gggcaattat ggataaagct gttataaaca tctgtatgca gccttttttg |
| 14761 | ttacatgcag gagataagct tttgactcat ttgggtaaat agcaaggggc atgattgcta |
| 14821 | gattgtatgg tgagagtata tttagtttta taagaaactg ccaaactgct ttccaaagtg |
| 14881 | attgtaccat tttgcgttcc catcagcaat gcattggttt ctttacagag tatttatttt |
| 14941 | cctttgctga cttattttaa cagcaggatt ctagcactag gtggaaaacg tatatgagtt |
| 15001 | tctgatgtgt gtcaggtact gtcctaggtg ctgaggttat aatagtgaat ccaacacaat |
| 15061 | ctctgtcttc atggagctta tgttctcatg tgaatgtttt tacacttcag agtttttatt |
| 15121 | tggttgagaa aagacttgaa atataacatc caaaaataaa atgtcagcct ggtgctgtgg |
| 15181 | cgcacgcctg tagtcccagt gctttgggag accaaggatg ggaggattgc ttgaggccag |
| 15241 | gactttgaga tcagcctggg taacatagtg agaccctgtc tctacacaaa ataaaatagc |
| 15301 | cgggcatggt ggcatgcgct tgtagtccta gctactctgg aggctgaggc aggagaattg |
| 15361 | cttgagccca ggagttcaag gttatagtga actatgatcg caccattgct ctccagcgta |
| 15421 | ggcaacagag caaaaccctg tatcaaaata aagtaaaata agcctgtcaa acccaaacca |
| 15481 | aatacatgtc aaacccagac ctgattctta ccacacacta tgccttctag taaaattctg |
| 15541 | gcctttgaat accaacaaaa tacttcataa cctgggaaac caactactct aacgtttgtt |
| 15601 | gctctgtctt gaaatactaa acaaaaccat ttacctgttc gtagacatat tttctttttc |
| 15661 | tagtttaaaa gtatcccatt ctgccatttt ccacctagtg ctagaatcct gctcttaaaa |
| 15721 | taagtcagca cagggaaata aatactctat aaagagacca aaagaccttt caaaataggc |
| 15781 | tatggccagg cccagtggct cacgcctata aatcccagca ctttgggagt ctgaggtcgg |
| 15841 | aggattactt gagcccagga gttctagact agcctgggca agatggcaaa accccatcta |
| 15901 | cacacacaca cacacacaca cacacacaca cacacacaca cacacacaca cacacacacc |
| 15961 | cccgctacct acctctctct ggggcatgcc tgtagtccca gctattctgg aggctgaggt |
| 16021 | gggaggatcc cttgagccca ggagtttgag gctgcaatga gccattatga caccactgca |
| 16081 | ctccagcctg ggtgaaagag ccagaccctg tctcataaaa taaataagta aataaataaa |
| 16141 | atatctacct taaagcagca agttttgtat ttgatgtaat gaattaacag accgctttat |
| 16201 | aatatctatc ttttaggata gtgtcagtct cttggaatgc tgtgcatgag tcatcattaa |
| 16261 | aattggtttt cattaaaaaa atttttttaa catatacaca tcttattgga gttttctttt |
| 16321 | aaaattcaaa aatgggaaca ttattttttc aaagccaagt aacagaagag tatataaaaa |
| 16381 | agtcaatttt gtgcatatct ttttacattt tctatgctca tgtaaaacat acatgactta |
| 16441 | tgtaattata cacatatgct ccagatctca tctgtattaa ttttttaaat tatactttag |
| 16501 | ttctgggata cacttggaga acgtgcagtt ttgttacata ggtatatata tgtgccatgg |
| 16561 | tgatttgctg cacccatcaa cccatcatct acattaggta tttctcctaa tgctatctct |
| 16621 | cccctagccc tgcacccctg aataggcccc ggtgtgtgat gtttccctcc ctgtgtccat |
| 16681 | gttttctcat tgttcagctc ccacttatga gtgagaacat gcggtgtttg gttttctgtt |
| 16741 | cctgtgttag tttgctgaga atgatggttt ccggcttcat ccatgttggt tttcattttt |
| 16801 | gtgatagtta attcttggac attaagggga ataattataa aacactgttg cttccatatc |
| 16861 | aagggaaatc actgaatgat taaaaaaaac ccaataagtg ttgtctgatt gtttaaacag |
| 16921 | ctttcattga ggtaaataag taaaacaaag ctgtgagaag tagggaatat ttaaacaatt |
| 16981 | tgtgcgcatg ccctcacctt tggggagaag tcttttctga aggctctctt ctgatttttt |
| 17041 | gaagcaactg tttaatcatt aaagaaaatt ataggtaaaa gacaaattct tgtgaacttt |
| 17101 | cgctcattgc tagattatta ggcagaacta ttttttaaaa atttaaatga aacaacttta |
| 17161 | tatttttaca tctttagatc atatcatttt gtaactatac tttagggaaa tagaggccag |
| 17221 | catattaaaa cagtaactag gggctgggca gggtggctca ttgcctgtaa tcctagcact |
| 17281 | ttgggaggcc gaggcgggca gatcagctga ggtcaggagt ttgaaaccag cctggccaac |
| 17341 | atagcgaaac cccatctcta ctaaaaatac aaaaattagc aggagaattg cttgaaccgg |
| 17401 | agaggcgaag gttgcagtga gctgagatca ggccattgca ctccagcctg ggagacaaga |
| 17461 | gtgaaactct gtctcaaaaa caaacaaacc aaaaaacaaa cacacacaca cacacacaca |
| 17521 | cacacacaca cacacacaca cacaaacaca caaaccagta actaggacta actatatgct |
| 17581 | gttttagatt gacagtattc tttttttcta aaagagaatg aatgccagca gatgtaggaa |
| 17641 | gtaccagagc taaaccattg cctggttaaa aggagtacct ctgcataata gttacttgtt |
| 17701 | gactcttttg taataaaata tttcaggcat gtaaaactag acagttaccc aagaaataac |
| 17761 | tgtgtatcca ccactccgta taagtaaaac agcccagcta gagtgaaagc ctgctatgtc |
| 17821 | cccttctctg gtcccattcc cctccctccc taccctggag ttaccagagt cctgaatttg |
| 17881 | gtgtgcatga tttgaagatt tctttctact tttattgtat gtatatgtgg atatatatat |
| 17941 | ttgtgtatat ataaatcact aaaaaagata ttattttgga tgtcttttca acttaaggaa |
| 18001 | tatcgtattc tgtgtaacct tctgcaactt gcttttcctc tcattgtttt gtttttggaa |
| 18061 | ttcatcttgt tgacacatgt aatttcagtt catttatgtt tccttttaaa aaatctttac |
| 18121 | tacttacgtt tacaatgact cattgttaca gtgcggtaga gacccttatg ccaaaaggaa |
| 18181 | gtaggtggca tttttcctta tttatataga atgtcacatg actgggggac tgttcgaaac |
| 18241 | gtgtatgaaa attaagaaca ccttcataga tagcagaatg cacacttata ttcttgagcg |
| 18301 | tgctagggga tgtttgagct aattaattga ctttcatggg ctttcttttt ttctcatctt |
| 18361 | ttattttttt gagacagggt cttgctgtgt cacccagact ggaatgcagc aatctgatca |
| 18421 | tgactcactg taccctcaac ctcctgggct caagcaatct tcctgcctca ggctcccgag |
| 18481 | tagctgggaa tacaggtgtg cagcaccaag cctggctaat tttcttt-tta aaaatttgtt |
| 18541 | gtggagatgg tgtctcatta tgttgcccag cctggtctca aactcttggg ctcaagtgat |
| 18601 | ctcccgcctc agcctcccaa agtgttggga ttacaggtgt gagccaccac accctgccat |
| 18661 | ttttcatctt tttaaaaatg gaacatctca aataagcaag tagatagaat aatgcattaa |
| 18721 | accctgctgc acttgccact caggcccaac aactgtcaat gcaaggttaa ttcaattcca |
| 18781 | tctacatttc tatctttgtt ccctctcacc tttgttatcc tgaagcaagt cgcaaacatc |
| 18841 | atatggttca tagggcttat ctttaaggaa ggaaccaggg tgggcagaag gaaatggaaa |
| 18901 | catgcagggg tttaggttgg ccagaaaagg tacctgtggc ctatggtcaa atggcaagac |
| 18961 | tgatgatggt tcaatactgt caagcctttg gctgtcatgg agatagacgt gcttgagaac |
| 19021 | tgatttagaa tagttgggaa acctgtaggg agattgaaaa agtaaccaac tgaagaccag |
| 19081 | gaatacaaga tggatttgaa tattgttcta gcttcacact cctaagtagg ggctgtccta |
| 19141 | ttttaagggt gtagacaatt ggttttgagg aaatcttggg ttgtttttgc taattattta |
| 19201 | taactttttt tttttttttt tttttttgag acagagtctt gctctgtcgc ccgggctgaa |
| 19261 | gtgcagtggc gcaaggtcgg ctcactgcaa cctccgcctc ccaggttcaa gcagttctcc |
| 19321 | ctgcctcagc cttctgagta gctgggacta caggcgccca ccaccatgcc tggctaattt |
| 19381 | ttatattttt tagtagagat ggggtttcgc catgttgact aggctggtct tgaactcctg |
| 19441 | acctcaggtg atctgcccac cctggcctcc caaagtgctg ggattacagg tgtgagccac |
| 19501 | cgcgcctggc ctatttataa ctttaaaaag agggtttggt tcctttccag ggtttttcaa |
| 19561 | actttctgtt tgtttcgtat aggcaagttt ctcatgtttt gtattgatgg cattctatta |
| 19621 | ttcataccta tctgttggtg ggcaactaat caacctgggt tgcgcaggac tgaaggattt |
| 19681 | cctgggggca tggaacttca gtcttcagac agtcctaggc aaactgggat ggtcaccccc |
| 19741 | tctcttggtt gctgcattag ttatctattg ctctataatg aattaccaca aacttagcag |
| 19801 | cttaaagcag cacacagtta ttatctccca gtttctgtgg gttcgggagt ctggacatgg |
| 19861 | ctcagttggg tcctcagctc agagtttcat aaagccacaa tcaaggtgtc tggagcttgg |
| 19921 | agtcctctgt ggaggtccaa cacttggttg cagaattcag ttccttgtga ttttagaact |
| 19981 | gaggctctca acttcgaggc ttcctgcagt tccctccaca tggccttctc cataggcaat |
| 20041 | tcacaacatg gctcttggct tcttcaaggc cagcaggaga gtgtctctct ccagttagct |
| 20101 | cagacacctt tgccatagtc cattagttag aagcaagtca caggttttgt ctgctctcaa |
| 20161 | ggggagggga ttatacaaaa caacagagac caggaggtga gaattttgcg ggggacacct |
| 20221 | taggatcttt ccaccacact tgctttccag gatttgtctg aagtagaaaa ttattctgtt |
| 20281 | gagccttttt gtggccagaa tatacatatg ttttgctcag ccactaattt agaattcctt |
| 20341 | tatcttttta agaaacattt tcatgcgtaa ttcatcctaa gtagaactgg caaatatgta |
| 20401 | acttcttagg tattttcaat tttagtcttt aggcaattgt ttttcctctt aactagtagg |
| 20461 | aggcaggaaa aaaaagtgga aaagcagatg caaatgccct gcctggctac tcctgcacgc |
| 20521 | agtgctctaa agcaggcctt ctcagcctgg gttagttcag gggtctgtga atttagatgt |
| 20581 | ggaaaagaac tacatctttt tttttttttt tactaactgc ccttccccca actgaaattg |
| 20641 | agcacttcct tctattatgc atgtaagcaa aaaaccacag cagttttttt tttttttttt |
| 20701 | tttttttttt ttttttttga gacagggtct tgctctgtca cccaggctgg agtgcaatct |
| 20761 | cacaattttg gttcactgca acctgtgcct cctgggccca aatgatcctt ccatctcagc |
| 20821 | ctcccaagta gctgggacta cacgtgcatg ccaccacacc cagctaattt ttgtattttt |
| 20881 | tgtagaaacg ggattttgcc atgttgccta ggcaacaggg ttcactgctt tcggtcaaat |
| 20941 | gacagcaatg ctgctggcaa gtccatggtc aggagtgaga ttccctcagg ctgctgtggt |
| 21001 | ccctcctcct ccagtctgtc ctgcaggaag ctcagagtga atggatgctc agctgtttcc |
| 21061 | tctgagacct caccagggac agttcctgca gtcccggctc tgagctctgc tcctgaccgt |
| 21121 | tttatctggc cctctcagct gtgcttcact gcaaaggagg aaggacaagg ccctctggag |
| 21181 | ccagtgccag ctgcttgtgg ggcctgtttt atttactact gctatctcta cttcagagac |
| 21241 | ttgggctagg acatagactt gaagaaaaaa gccattttaa cagacttgag aaactcagcc |
| 21301 | atcctcataa taccattcag aaccaggaga ggccattatt aacactaggc tagtaggaaa |
| 21361 | tgttcacttt gactgaattc atttgtaaca ctttgccact ggtttacaag aaaaatactt |
| 21421 | tggttcagtc tcctggaggt aggtaattaa atgtttcacc tgtgctaatt ttccaaataa |
| 21481 | ttaaagtgaa tcccataaat gccagcaatt aagaattgtt gtttttggcc aagtgtggtg |
| 21541 | gctcatgcct gtaattacag cactttggga ggttgaggcg ggcggatcac ttgaggccat |
| 21601 | gagtttgagg ccaacctggg taaaataggg agacgctgtc tctacaaaaa atacaaaaat |
| 21661 | cagctgggct tggtggaatg tgcctgtagt cctggctact tggtaggctg aggcaggaga |
| 21721 | attgcttgaa tccgggaggt agaggtttca gtgagccaag attgtgccac tgtactccag |
| 21781 | cctaggcgac agaggcgaga ttctgtctca aaaaaaaaaa aaagttgttt cttaagtgca |
| 21841 | gaccttctga gatgtgtagt gcttgcccca tgccttcaaa agaacatatt taatacaata |
| 21901 | atctctttgt ctctccttta cttcaaagtc agccttgtga aaaacagatg atgatatacc |
| 21961 | aacagttttt tgctcttgga ggtgtttggt gtgctctgct ttttgagttt tgttttttgg |
| 22021 | acttctttaa aattatttta aaatgtttta actcctctcc tttgcatact ctctggtacc |
| 22081 | cttatagtcc caatactgag taaagcatgt aaaatttagc atgcagatgt tttagcctct |
| 22141 | tttatttttt gttcgagatc cagatttttt agtctcagca tgactgacac tttggggctg |
| 22201 | tatcattctt tgttgtggag gactgtcctg tgcattgtag gttgtcaagt aacatccctg |
| 22261 | gtctctaccc acaagatacc agtagctatt cttctcctga ttgtgacaac aaaaaatgtt |
| 22321 | tccagacatt gtcagatgtc tcctcggggc aaaatttagt tctacatata tgatgataaa |
| 22381 | aaccatggta gcagccaaca tttgttcact atcatcccca ttttgaagaa agcaaaaacg |
| 22441 | gaggcactga gggtgaggtt cacttacttg cccaaggtca catagataaa agtcacttct |
| 22501 | tgaaatactt ttcataatat atatgggttg aatctcagtc ttaattgagg aggcagacta |
| 22561 | aaaaagaatg taaaaacggg gagtttcctg atgatgtctc aattgagagg ctattgcttt |
| 22621 | catagtgatt ctgtgtaaat gctggaattc ttgcttaaac ggggtgattc catgggcatg |
| 22681 | tgtctgagta aaggacttgg atcatttcta agaagcttct gtggcatggt gaagcagaac |
| 22741 | ataagacagt ctaggatcaa ggataactga tcaaactttg aaaccaagga agccatagga |
| 22801 | aattggggta aggctatgta tcacttaggg tcccacagga aatggcatgc tcaaagggtt |
| 22861 | ttagtgaaaa caggaatgtt gaggcacgaa gggactagca gcaggaagct attgccattc |
| 22921 | ctagacctgg tggggcaaag ggaggggagg gaatgtcgtt gctggagctg tgaaaggtac |
| 22981 | tccctctaca ggaacaatag tcatgaaaga atacagcctt tgctagaact atggccagag |
| 23041 | aggggccagt gggatgaggg gatgggagag aagtaaccaa tttctgtttc ctgccctctc |
| 23101 | atttgctgca gtgccttttg ttgttctaac ccaactagaa gccacaggta acagagcctg |
| 23161 | ggtgttgaag cttattgagg ttagcctctt gcgctgagtg gatggaaagg gctgagagtg |
| 23221 | gttcaagggg gcaaatagag caacagccag cacaggctat gagcaggagt gggaaacact |
| 23281 | accctggatc ctaggaagga tggggcatgg ccagtgatag gagcaatgaa agtctttttg |
| 23341 | tgtgaaaaag agcctgggga tacagacctc catgaaggag aagagagggt tggataggag |
| 23401 | tgactggttc atctgaggtt cttcctattt ctccaaggaa gccttgagga atgtggggtt |
| 23461 | gtctgtttgc atttggatat ggaagtatgt gtgtgtgtat acttttattt attttgagac |
| 23521 | agggtcttgt tctgtcaccc aggctggagc acagtggcac catcacagct cactgcagcc |
| 23581 | tctacttcct gggcttaagt ggtcctccca cccaggcccc cccaggtagc tgggaccaca |
| 23641 | ggcatgtacc actatgctcg gctgattttt aatattttgt agagacaggg tctcactata |
| 23701 | ttcccctggt tggcctcact cctgggctca agtgatcctc ccaccttggc ctccagaagt |
| 23761 | gctaggaata caggtgtaag ccactacacc cagtctgttt gtatactttt tggtcagagt |
| 23821 | gaggctttcc gtgagttgga gtgtttgtcc ttattcatga cactgggaaa catacaacag |
| 23881 | tggagtgacc ctcttgcaga ttctgctagg agtagctaca tatggtggga aaggttgcta |
| 23941 | tgggaatgga gataaattca aRgcagtaga gagtgcttta taatgtttgg gggtatggcc |
| 24001 | aagcattagt tttgcttttt ccttcccagt aggatacgat ttctgtagca attcttcctt |
| 24061 | acccaaaaat agctatatgt gctcccaaaa agaaaaagcc caggaccaga tgacttcatg |
| 24121 | gctgaattct accaaacatt caaagaagag ttaatactaa tccttcttaa actcttccaa |
| 24181 | aaaacagaag tagagagagt acttccaaac tcattttttg aggccagcat caccctgatt |
| 24241 | tcaaagccag acaaaagata ccaagatacc ataagaggag aaaaccacag gttagtatct |
| 24301 | ctgttgaaca tatatgcaaa aatcctaaat aaaatactag cagaccaaat ttaacaacac |
| 24361 | ctcaaaaaga tgatacacca tgaccaaagt ggcacttatc cctgagatgc aagattggtt |
| 24421 | taacgtatat aaaacaatca gtgtaatatg ccacattacc aaaataagag gtaaaaaaca |
| 24481 | agccaggtgt ggtggctcac acctataatc ccaacacttt gggaagctga ggtgggagga |
| 24541 | ttgcttgagg ccaggagttc aagaccagcc tgggcaacat agtgagacac tgtctctaca |
| 24601 | aaaaattaaa aaaaaatgag ctgggcatag tggcatgtac ctctagtccc tgctacttgg |
| 24661 | gaggctgagg cagcaggatc gctggagccg aggattttga ggctgcagtg agcactgatt |
| 24721 | gtgccaccgc actccagttg ggcagtacag caagaccctg tcttcaaaga aaaaaaaaga |
| 24781 | gagaaaaacc catgatcatt tcaatacatg tagaaatagc atttgacaaa gttcaacagc |
| 24841 | ctttcgtgat aaacattttc aacaaaataa gtttaaaagg gaatttcctc aacacattaa |
| 24901 | agaccattta tgtagagtcc acagctaata tcataatcag tgggagaaaa ctgaaagctt |
| 24961 | tttttctaag atctggtaca aagcaaggat gcccactctt gccactttta ttgaacacag |
| 25021 | cactggaagt actaacatga gctatcagac aataaaaaga gataaaaggc atctaaattg |
| 25081 | ggaaagaaga agtaaaatta tccctgtttg taaatgtcat gatcctatat gtagaaaacc |
| 25141 | ctggactcca caaaagccct cttaagacta ataaatgagt tcagtaaagt tgcatgatac |
| 25201 | aaaatcagcg tacaaMaatc agttggaaat caagaaaact gtcccattta tgataacata |
| 25261 | aaaataataa aatattcagg aatacattta atgaaggaag tgaaagatct gtacattcaa |
| 25321 | cattataaaa tattgatgaa agaaattgaa gacacaaata aatggaaaga tatgccatgt |
| 25381 | ttatgcattg gaaaaattaa tagtgttaaa ttgtccacac tacccaaaac aatatacagg |
| 25441 | ttcaatgcaa tccctatcaa aataccaatg gcatttttca cagaaataga aaaaacaatt |
| 25501 | ctaaaattca tatggtgtga caaaagacct agaatagccg aaacagtctt gagaaacaaa |
| 25561 | aacaaggttg gaggcatcac acttccttag attataatac agtataaagc agtagaaatt |
| 25621 | atgatagtat atcctggcat aaaaacagac atatagatca atagaataga acagagatcc |
| 25681 | gagaaatata cagtatatat acagaaatcc aagtatatac agtcaactaa tttttgacaa |
| 25741 | ggccaccaag gagacacagt ggggaaagga tagtctcttc aataaatgat attgggaaaa |
| 25801 | ctggatattc acaggcaaaa gaatgaagct ggatgcttat atacaaaaat caactcaaaa |
| 25861 | tgtgtcttta aaatatacaa caaaacctga aaatggaaaa gaagacctaa atgttaataa |
| 25921 | ctcaaaccat tgaactccta gaagaaaaca taggggaaaa gctccttgat atcggccttg |
| 25981 | gcaatgattt tttagacatc acaccaaaag cacaggcaac aaaagcaaaa agaaacaggt |
| 26041 | ggtactacat cagactaaaa agcttctgca tgacaaaggg aacaatccgc aaataaaagg |
| 26101 | gcagcctatg aattggaaga aaatatttgc gacccatata tatgataagg agttaatatc |
| 26161 | aaaaatatat aaggaactcc cagaactcaa tggtaagaaa atgaataacc cgattaaaaa |
| 26221 | ataggcaaag gacttgatta ttcattttcc caaagaagcc atacaaatgg ccagcaggtt |
| 26281 | gtgaaaaggt actcagtatc actaatcata agggaaatgg aaatcaaaac cactatgaga |
| 26341 | tatcagctca caccttttag aatggctatt agaaagacaa cagataacaa acgttagggt |
| 26401 | gtggagaaaa gagaaccctt ttatactttt gatgggaatg tagattggta cagccattat |
| 26461 | ggaaaatggt atagaagtgc ctcaaaaaat taaaagtaga actaccatat gatccagcaa |
| 26521 | ttcctctgct ggatatgtac ctaaaggaaa tgaagtcagt gtgtcaaaga ggtatctaca |
| 26581 | ctcctgtgtt cattgcagca ttattcgcaa tagccaagat atggaaacaa tttaagtgac |
| 26641 | tgttgatgga tgagtggcta aaaatatcgt gggatacaca cacacacaca cacacacaca |
| 26701 | cacacacaca cacacacaca cacagatgac agaatattat tcagccttaa aaaaggagat |
| 26761 | catgctattt gtgacaacat ggatgaactt atgctaagta aaataagtga gacacagaag |
| 26821 | gaaaaatact tcatgatctt atttatctgg aacctgaaaa aaagagtcaa atatatagaa |
| 26881 | acagaataga accacggtta ccaagggtgg gaatggggag gaaataaggc aatgtaggtc |
| 26941 | aaagtgtaca aacttgcagt gatgtaggct caataagtct agagatgtac agcaggagga |
| 27001 | ctatagttaa caatattgta ttgtatatgg aaaatgtgct aagagtagat tttaggtgct |
| 27061 | cttacctctt ctccccacaa aggtaactat ggaaggtgat gagtatgtca gattgcttac |
| 27121 | tggtagtaat catttcacta tggatatgta tatcaaaaca tcatgtcata catgttaaag |
| 27181 | ttatgccata aaaatagcta tatgtgggcc aggccaggca tggtggctta tgcctgtaat |
| 27241 | accagcattt tgggaggctg aggcaggagg attgcttgag gccaggagtt taagaccagc |
| 27301 | ctgggcaacg tagtgagaaa cctgtctctc caaaaatgtt tttaaaagtt atctgcgtgt |
| 27361 | ggtagtgcat gcctgtgatc ccagctactt gggaggctga ggcaagagga ttgcttgagc |
| 27421 | ccgaaaggta gaggctgcag tgacctgcga ttgcaccact gcactccagc ctgcgtgaca |
| 27481 | gagtgagacc ctgtctctta aaaaaaaaaa agattaaaaa atagttgtat gtgtatatgt |
| 27541 | atacgttcag gatttgttct atttgttttc tttttctttt ttttcatagg cagtggtcaa |
| 27601 | tggcctaagg atttctttat acattgctca tatccactaa ggctctattg agcaaaccac |
| 27661 | gccttaaaaa tcttacaatc ctcttcatat attttaaagg caaatgcaaa gaaatagctt |
| 27721 | tgttctgttt tgcattcttc atgggttaac atatcttctg gaaaacttgt tgctatggca |
| 27781 | atgggatatt atctatcttg gtaatgtact acataaaaat agccctttgt gtttgagaat |
| 27841 | gatgttagtg acaggtggtt gggtgaatgt atagcaggct gctttgtcaa gaatagtttc |
| 27901 | ccaacattcc attgtttaaa gttgtgaaat caaaatgagt ataatttttt tctctctcaa |
| 27961 | aatgtaattt tatctggaaa aggcaggcag tggtgctctg tttaattatg aattacaagc |
| 28021 | cttcctttgc aggtcaggtc aacatgatga ttgaagccac ctctcactgt aataagtaaa |
| 28081 | cgacttctct attctttctg tcagggtgtg cagcacaagg ttgcgctggg ggggccagta |
| 28141 | gggataattt tcccattctt gaacacatcg ttcttcctaa gcacctcgta cttataaacc |
| 28201 | aagaagtagg aagtggtcag gtggaagaga tgagctctga gctgttaatc aaatccgaat |
| 28261 | ttcttgtagg ttcttaggtg aaacagttcc ttgggaaagg tcagatttcc atgttgcttt |
| 28321 | tagtattgaa ctcacacata tagccaagga tagaatctga cccaaatctt aatttttcct |
| 28381 | ttgaaaaatc ctttgggcaa ttttttttta atttaattta attttattat tattattatt |
| 28441 | attttttatt gatcattctt gggtgtttct cgcagagggg gatttggcag ggtcacagga |
| 28501 | cagtagtgga gggaaggtca gcagataaac aagtgaacaa aggtctctgg ttttcctagg |
| 28561 | cagaggaccc tgccgccttc cgcagtgttt gtgtccctgg gtacttgaga ttagggagtg |
| 28621 | gtgctgactc ttaaggagca tgctgccttc aagcatctgt ttaacaaagc acatcttgca |
| 28681 | ccgcccttaa tccattcaac cctgagtgga tacagcacat gtttcagaga gcaccgggtt |
| 28741 | gggggtaagg tcaccgatca acaggatccc aaggcagaag aatttttctt agtacagaac |
| 28801 | aaaatgaaaa gtctcccatg tctacctctt tccacacagt cacggcaacc atccgatttc |
| 28861 | tcaatctttt ccccaccttt cccccctttc tattctacaa aaccgccatt gtcatcatgg |
| 28921 | cccgttctca atgagctgtt gggtacacct cccagacggg gtggtggccg ggcagagggg |
| 28981 | ctcctcactt cccagtaggt gccgccgggc agaggcgccc ctcacctccc ggacggggcg |
| 29041 | gctggccgga cggggggctg acccccccca cctccctcct ggacggggcg gctggccggg |
| 29101 | cagaggggct cctcacttcc cagtaggggc ggccgggcag aggcgcccct cacctcccgg |
| 29161 | acggggcggc tggccgggcg gggggctgac ccccccacct ccctcccgga cggggcggct |
| 29221 | ggccgggcag agtggctcct cacttcccag taggggcagc cgggcagagg cgcccctcac |
| 29281 | ttcccggacg gggcggctgg ccaggtgggg ggctgacccc gccacctccc tcccggacgg |
| 29341 | ggcggctggc cgggcagagt ggctcctcac ttcccagtag gggcgcccgg gcagaggcgc |
| 29401 | ccctcacctc ccggacgggg cggctggccg ggcggggggc tgaccccccc acctccctcc |
| 29461 | cagatggggc agctggccgg gcggggggct gaccccccca cctccctccc ggacggggcg |
| 29521 | gctggccggg tggggggctg acccccccac ctccctcccg gacagggcgg ctggcagggc |
| 29581 | agagtggctc ctcacttccc agtaggggcg gctgggcaga ggcgcccctc acttcccgga |
| 29641 | cggggcggct ggccgggcgg ggggctgatc cccccacctc cctcccagac ggggcggctg |
| 29701 | gccgggcggg gggctgaccc ccccacctcc ctcccggacg gggcggctgg ccgggcgggg |
| 29761 | ggctgacccc cccacctccc tcccggacga ggtggctgcc gggcggagac gctcctcact |
| 29821 | tcccagacgg ggtggctgct gggcggaggg gctcctcact tctcagacgg ggcagctgcc |
| 29881 | gggcggaggg gctcctcact tctcagacgg ggcggttgcc aggcagaggg tctcctcact |
| 29941 | tctcagacgg ggcgtccggg cagagacgct cctcacatcc cagacggggt ggcagggcag |
| 30001 | aggtgctccc cacatctcag acgatgggcg gccgggcaga gacgctcctc acttcccaga |
| 30061 | tgtgatggcg gccgggaaga ggcgctcctc acttcctaga tgggatggcg gctgggcaga |
| 30121 | ggctgcaatc tctgcacttt gggaggccaa ggcaggctgc tgggaggtgg aggttgtagt |
| 30181 | gagctgagat cacgccactg cactccagcc tgggcaccat tgagcactga gtgaacgaga |
| 30241 | ctccgtctgc aatcccggca cctcgggagg ccgaggctgg cgggtcactc gcggttagga |
| 30301 | gctggagacc aacccggcca acacagcgaa tccccatctc caccaaaaaa atacgaaaac |
| 30361 | cagtcaggcg tggcggcgcg cgcctgcaat tgcaggcact cggcaagctg aggcaggaga |
| 30421 | atcaggcagg gaggttgcag tgagccgaga tggcagcagt actgtccagc ttcggctcgg |
| 30481 | catcagaggg agactgtgga aagagaggga gagggagacc gtggggagag ggagagggct |
| 30541 | gggcaatttc ttagagcagt aactctcaaa cattttgtcg tcatcatctc acttttcttc |
| 30601 | tacttatcaa aatgagtatt gatttaattg taatgagtaa ataacagctc cctgctactg |
| 30661 | aaatgaaatt tactctagct ttgagaattt gagagaccct cttaagaaaa taccacagct |
| 30721 | ggaaatttga gacctaaaag taatacaatt tgagtatctc atatctgaaa tgcttgggac |
| 30781 | cagaagtgtt ttggattttg gattttttca gactttggaa tatttgcgtt tacataatga |
| 30841 | gatctcttgg ggatgggacc caagtctaaa cacgaaattc attaatgttt tatatacgcc |
| 30901 | ttatacacat agcctaaagg taattttata caatatttta aataagcttg tgcatgaaac |
| 30961 | aaaattttga ctgtgttttg actgtgacct gtcacataag gtcaggtgtg gattttttca |
| 31021 | tttgtgactt catgtcaaca ctcagaaact ttcacatttt ggagcatttt ggatttcaga |
| 31081 | ttagggatgc ttaatctgca gctctaattc tgatatgcac ggtactactg cagagatgga |
| 31141 | accattgcct ttgtcttttc tgttggagtc agtatagata tcttgctact aataatatca |
| 31201 | gaagagagtg ttgagtggtt tgctgggtgc ccaatactac ttttagaaca ttttatgtat |
| 31261 | taactaattt attcctgtaa acattcttga cagtttttgt gactatttcc caaatgaggc |
| 31321 | gtacttgtta gcatgtcttt ctgtttgtgg tgagtaagca ttcttgtgta ttggaagact |
| 31381 | tgcttactag catgtcacag tgttcttcat attgaaagca ttcttctatc aggagttgac |
| 31441 | gtaattcagc ctgtgggcca aatccatcct gccacccatt ttttgtgtat tttataagct |
| 31501 | aagaatggtt tctgcatttt taaatggttg ggaaaacata aaaagaataa tttgtgacat |
| 31561 | gtgaaaatta catgaaattt aaatttcagt ttccataaac agttttattg gaacacagct |
| 31621 | ttactcattt gtttacatat tgctaaggct gcttttgaac tagagtggca gagttgagta |
| 31681 | gttgtaatag agaccatata gctcccagac cctaagaaaa agtttgccaa tcccttctct |
| 31741 | atattcttct gtatctagaa tggcagatgg tcctcaataa taacagcaga ttggtcaggt |
| 31801 | gtcatagttt atgcctgtaa tcccagcatt ttgagaggcc aaggtgggag gatcacttga |
| 31861 | gctggagttc aagaccagcc tgggcaacac agtgagacct agctctgcaa aaattagcca |
| 31921 | ggtgtcatgg tgcacgctgg tggtcccagc tacttgggag gctgaagtgg gaggattgct |
| 31981 | tgagcctggg aggtcaaggc tggagggaga cgtgatcaca ccattgtgct ccagcctggg |
| 32041 | tgacagagcg agaccctgtc tcaaaaatat ataaacagat tggatttatg tacaggttca |
| 32101 | tatatatatt aattttcttt tggtatgcaa tatatttttc tttcttccac taaacgtttc |
| 32161 | tcttgtcata agcttgatgt catgtactat ggcatatctc aaatatacgt gatttcagct |
| 32221 | ctttgcatac ccatattggt tttctttttt ttttttttaa ttgatcattc ttaggtgttt |
| 32281 | ctcgcagagg gggatttggc agggtcacag gacaatagtg gagggaaggt cagcagataa |
| 32341 | acaagtgaac aaaggtctct ggttttccta ggcagaggac tctgcggcct tccgcagtgt |
| 32401 | ttgtgtccct gggtacttga gattagggag cggtgatgac tcttaaggag catgctgcct |
| 32461 | tcaagcatct gtttaacaaa gcacatcttg caccgccctt aatccattca accctgagtg |
| 32521 | gacacageac atgtttcaga gagcaccggg ttgggggtaa ggtcacagat caacaggatc |
| 32581 | ccaaggcaga agaatttttc ttagtacaga acaaaatgaa aagtctccca tgtctacctc |
| 32641 | tttctacaca gacacggcaa ccatccgatt tctcaatctt ttccccacct ttcccccctt |
| 32701 | tctattccac aaaaccgcca ttgtcatcat ggcccgttct cagtgagctg ttgggtacac |
| 32761 | ctcccagacg gggtggtggc cgggcagagg ggctcctcac ttcccagtag gggcggccgg |
| 32821 | gcagaggcgc ccctcacctc ccggacgggg cggctggccg ggcggggggc tgacaccccc |
| 32881 | acctccctcc cggacggggc ggctggccgg gcggggggct gaccccccca cctccctccg |
| 32941 | ggacggggtg gctggctggg cagaggggct cctcacttcc cagtaggggc ggccaggcag |
| 33001 | aggcgcccct cacctcctgg atggggcggc tggccgggcg gggggctgac ccccccacct |
| 33061 | ccctcccaga cggggcggct ggccgggcgg ggggctgacc ccccccacct ccctcccgga |
| 33121 | cggggcggcc ggccgggcag aggggctcct cacttcccag taggggcggc cgggcagagg |
| 33181 | cgcccctcac ctcccggatg gggtggctgg ccgggcgggg ggctgacccc cccacctccc |
| 33241 | tcccggacgg ggtggctgct gggcggagac gttcctcact tcccagacgg gatggctgcc |
| 33301 | gggYggaggg gctcctcact tctcagacgg ggcggctgcc gggcggaggg gcccctcact |
| 33361 | tctcagacgg ggcggctgct gggctgaggg gctcctcact tttcagacgg ggcggttgcc |
| 33421 | aggcagaggg tctcctcact tctcagacgg ggcggctggg cagagacgct cctcacatcc |
| 33481 | cggatggggc ggcagggcag aggcactccc cacatctcag acgatgggcg gccgggcaga |
| 33541 | gacgctcctc acttcctaga tgggatggcg gccgggaaga ggcgctcctc acttcctaga |
| 33601 | tgggatggcg gccaggcaga gacgctcctc actttccaga ctgggcagcc aggcagaggg |
| 33661 | gctcctcaca tcccagacga tgggtggcca ggcagagaca ctcctcactt tccagacggg |
| 33721 | gtggcggccg ggcagaggct gcaatctcgg cactttggga ggccaaggca ggctgctggg |
| 33781 | aggtggaggt tgtagcgagc cgagatcacg ccactgcact ccagcctggg caccattgag |
| 33841 | cactgagtga acgagactcc gtctgcaatc ccggcacctc gggaggccga ggctggcgga |
| 33901 | tcactcccgg ttaggagctg gagaccagcc cggccaacac agcgaaaccc cgtctccacc |
| 33961 | aaaaaaatac gaaaaccagt caggcgtggc ggcgcgcgcc tgcaattgca ggcactcggc |
| 34021 | aggctgaggc aggagaatca ggcagggagg ttgcagtgag ccaagatggc agcagtacag |
| 34081 | tccagactcc cacggcatca gagggagacc gtggaaagag agggagaggg agaccgtggg |
| 34141 | gagagggaga ccgagaggga gagggagaga gggagaggga gagagggaga gggagagagg |
| 34201 | gagagggaga gatttggttt tcataataat cataaccctt tccaaatgag ggagagtaat |
| 34261 | gaattttatc attttattag ttagtgtgct tcctttgcta gatcaggaat tatatctcct |
| 34321 | agtaattata accctctatt tattattttt aactatattg ctttctactt ttgggctttc |
| 34381 | agccaacgtg taaatattga cagtttaata atctgaacca agccacctgg actgattgta |
| 34441 | atattttaat ttaataaatc ttgcatatgg ttaagattac atcttctaca ttcatcagat |
| 34501 | tggatttaat gcactcacca taatcaactc tggaacattc ttgcaattat tattgttatt |
| 34561 | tttaaaggtt ttctcctttg tctgttgttt tcccactttt tttttttttt tttaagacgg |
| 34621 | aatctccctc tgtcacccag gctggagtgc agtggcgtga tctcggctca ctgcaacctc |
| 34681 | cgccccctgg gttcaagcga ttctcctgtc tcagcctcct gagtagctgg gactacaggc |
| 34741 | gtgtgccacc acgcccagct aattttttgt atttttagta gagatggggt ttcaccatgt |
| 34801 | tggccaggct ggtctcaaac tcctgacctc aagcagtctg cctgcctcgg cctcctgaag |
| 34861 | tgctgggatt ataggcgtga gccactgtgc ctggcctgtt ttcccacttt ttaaagtaac |
| 34921 | agccttgttg agatgtaatt cacataccat atagtcacct atttaaagtg tacaattcag |
| 34981 | tggttttagt atattcacag aattgtgcaa ccatcacaac tatttccgga atatttttgt |
| 35041 | cacccaaaaa agaaacccca tactcactag caatcacccc cagttccacc attcctaggc |
| 35101 | agccatggat ctactttttg tctatggatt cgtctattcc agacatttca tagaaatgaa |
| 35161 | accacatagt gtgtggtcat ttgtgattgt cttctttctc ttagcgtgtt ttaaatgtag |
| 35221 | cacgtatcag cacttcttat tgccagttct gctgtatggc tataccacat tttatttatc |
| 35281 | aattcatgag ttgacagaca tttgggtcat tttcactttt tgcctcttat gtataatgct |
| 35341 | actctgaaca cttgtgtata ggtttttgtg tggacatatg tttttatttc tgtcgaatat |
| 35401 | atacctagag gtggaattgt tgtgtcgtat ggtaactcta tgtttaacct tttgaggaac |
| 35461 | tgccaggctc ttttgcaaag ctgctgtact atttgacatt cttgtgagca atatatgagg |
| 35521 | gttccagttt ccccacatcc ttaccaacgt ttgttattgt ctatcttttt gattatggct |
| 35581 | atcctagtgg gtgtataatg gtatctcatt gtggttttag ttggaatttc cctgatgact |
| 35641 | aataatgttg catgtctttt cacgtgctta ttggccactt gtatatcttc tttagagaaa |
| 35701 | tgtgtattta gatcgtttgt ccattttaaa ttgggttgtc tttataaaat gttgaattgt |
| 35761 | aagagttctt tatatattct ggattcaata catatgatac aatacatata atacatattt |
| 35821 | tagtcatcta tcagataaat gatttgcaaa aattttcttc cattttgtgg gttgtctttt |
| 35881 | tactttcttg atggtgtcct ttgaagcatg aaagctttta attttgatgg agtttattta |
| 35941 | ttgttttcta ttgtcacttg tgtttttggt gtcatattta agaaaccatt gcctaatcca |
| 36001 | agatcacaaa gatttcactt atgttttctt ctaagagttt tgtagtttta gctcctatat |
| 36061 | gtcagtcttt gattcatttt gagttaattt ttgtacgtgg tgtgaagtag gggtacaact |
| 36121 | tcatactttt gtatatggat atccacttgt cttagcatca tgttttgaac aaactgttct |
| 36181 | tttcctaatg aattgttttg gaacccttgt gcaaaaccaa ttgaccttat gtaaatgtaa |
| 36241 | gggcttatgt ctagactgtg tcagtatcac actgtcttga tttgtttaat ctatttttga |
| 36301 | ggcagacttt cgctccctga tctaaaatta aaatgtttca caattgatga tgtcttatat |
| 36361 | gcatcaaaac ttgcagactg ggtgtaagtg gcttgagaaa atatattgga gactaaagtg |
| 36421 | gggctgttgg agactcatag gaatcaaata gttagaaggt gggggagtga gagaggcagt |
| 36481 | aagttggatg cttcccaggt gtttagcagc catcccaaag tggcagtcaa aaataggcta |
| 36541 | gctttacata atttcaaagt taggtactga tggttccagt ttctttttat ggattctttt |
| 36601 | aaaaagtgct gtatcaccaa atgctgttga tggcatagaa gacaacactg tggaaaaatg |
| 36661 | tggacattga ccagtctgag tcaaaaagat ggacagatat tcagttctaa atgtgaggaa |
| 36721 | tttttaggaa aaccttgagc agttaatttt gcttatttcc ctttctatgt gtatatgaat |
| 36781 | gataaatgaa aagtctaaaa gagctccttt aataagcctg agataaatgc taagtcgtag |
| 36841 | actactgcat catatcttat ttggcttttt tttagtgcca cctaaaatac tggtgtttta |
| 36901 | gctttgagat gctcttatcc tcctgcttcc agctctatct acacctcaga aggagctggg |
| 36961 | tcctttccca gagtctattg tataaattaa ccttcttcct gtagtcactc tatgtccatc |
| 37021 | tgtttcccag ccactttaat tttgttcttt ttcctttctc ttgtgcttgt agatttaagt |
| 37081 | cttaaaaaca agccaggtat tattgtttat agtggaattt tgagtaggag tggaggttaa |
| 37141 | cctgtacatc aatctagtag gaatattcat tcaatcactc aagggatgag cttatggaaa |
| 37201 | agttacaaaa atagtagcat tcctttatat tcctcaccct gctcccttaa tgttaacatt |
| 37261 | ttatataaag gatcaagaat aggaaattag tcttggtaca gtattattaa ctaaactaca |
| 37321 | gagcttattt gaattccatc agtttttcca ctggtggtct atttctgttc taggatcctg |
| 37381 | tccaggatcc catattgcat ttagttatta tttctcctta gtctcatcca gtttgtaata |
| 37441 | atttcttaat ctttaatgac acctttgaag agtacagatc aatattttgt agaatgttcc |
| 37501 | tcaatttgta tttgtttgat gctttcttat gaataaggtt gttcattttt ggcaagaata |
| 37561 | taatgatgca tatttttcag tgtatcatat caaggagttc atgatgttga tgtgtcttgt |
| 37621 | tactggtgac atttaccttg attccttgtt taaattgctg tctgcagggt ttctctacag |
| 37681 | taaaattatt actttttctc tttatggtta aaatatcttg ggggagatgc tttgagacta |
| 37741 | ggcagatatc ctatttatcc tcaaattttt gtccattgat ttttttcatt ctttggtgga |
| 37801 | tcactctgta tcatagagta tttgtgacaa atatttgatt agcagaaata aagctatgcc |
| 37861 | ctacaagaag caaagatcaa ttacattgaa atcctattgt attggttaat ataacgctag |
| 37921 | cagctgtgtt cctcaacttt caggagctta acacaacaga tgcttttctt ctcaatatgg |
| 37981 | gcatttctgg ttgtgtaggc agttttctgc catgttaatt caagtatcca gactaaactt |
| 38041 | tttatactga agtaacttca aactgacaga aaatagggat gcaagaatag taccaggaaa |
| 38101 | tcctgtatac cctttactag atttacttaa catttgctac atgtgcctta tgattttctt |
| 38161 | tgtctattca tatatgtata tgtattttat atatatttta tatatctgta tttatatatt |
| 38221 | gattgccata tactaaatca cttgagatta aatcacatac atcatgttct tctatgcctt |
| 38281 | aatagtttaa acttaatata tttatttgtt taaatctagt atacagtttc agaattgcta |
| 38341 | acccatatct gtttgagaaa cactttcact aactggatta cagcatttat acaattcttt |
| 38401 | tattctttag ttttgcaata tctagtcaag atacgatttt ccagtttccc actgaagtat |
| 38461 | taagtttgaa aactgtacat aagcacactg ttattcaaat ttaggaaatt taatatcagt |
| 38521 | actttagtat gcagtctgta ttccagtttc attaattatc cccaaaatgt tcttcagtgg |
| 38581 | aatatttttt ttctcctgat acagtatcaa gtccaggatc atgtattgaa tttatttgtc |
| 38641 | atgacttttt agtttccttt gatctggaac agatcttata agcctttctt tgtctttcat |
| 38701 | gacatcgaca ttttgaacaa tgacaaattt atgacattgt tttgtagaat gtaccaaatt |
| 38761 | tgggctgggt gctgtggctc atgcctacaa tcctagcact ttgggaggct gaggcaggtg |
| 38821 | gattgtctga gttcaggaac tcgagaccag cttggccaac atggtgaaac cccatctcta |
| 38881 | ctaaaaatac aaaaaacaaa aaaaattagc catggcacct ataatcccag ctactcagga |
| 38941 | ggctgaggca agagaatcgc ttgaacccgg gaggcagagg ttgcagtgag ccgagattgc |
| 39001 | accactgcac tccagcctgg gcgacagagc tagatgctcc atttcaaaaa aaaaaggaac |
| 39061 | gtaccaaatt tgggtttggt tgatggctcc tcttgtttag attgagatta tgcattttta |
| 39121 | aaaaaacttt taggttcagg ggtacatgtg ctgatttgtt gtataggtaa attcttgtca |
| 39181 | cgggggtttg ttgtacagat tatttcatta cccaggtact aaacctagta tgcagtagtt |
| 39241 | ttttttttct cttctccctc ctgccaccct ccatcctcca ataggcctca gtgtctgttg |
| 39301 | ttcttctctg tgtgtccatg tgttctcatc acttagctcc cacttacaag tgagaacatg |
| 39361 | cagtatttgg ttttccgttc ctgcattagt ttgcttagga taatggcccc cagctccatc |
| 39421 | catgttcctg caaaggacat gagctcattc ttttttatgg ttgtgtagta ttccatggtg |
| 39481 | tatatgtacc acattttctt tatccagtct atcattgatg ggcatttagg ttgattttat |
| 39541 | gtttttgcta ttgtgaatag tgctgcaatg aacatacgct tgcatgtgtc tttatggtag |
| 39601 | aatgatttat attcctttgg gtctataccc agtaatgggg ttgctgggtc gaatggtatt |
| 39661 | tccgttttta ggtctttgag gaatcgccac tctgttttcc acaatagttg aattaattta |
| 39721 | cactcctacc agcagtatgt aagcattcct ttttctctgc aacctcactg gcacctgttt |
| 39781 | ctttttgact ttttagtaat agccattctt actgatgtga gatgttatct cattgtggtt |
| 39841 | ttgatttgca tttctctaat gatcagtgat gttgtggttt ttttttcatc tgctcgttag |
| 39901 | ccacgtgtat gtcttctttt gaaaagtgtc tgagattatg catttttgtc tggaatactg |
| 39961 | cataagtgac atgtccttct caggcaatgc atcaggaggc acatgatgtc tcaacccctc |
| 40021 | attggtgatt gtaattttga tcatttggtt aaattactgt ttgcttaagt gttaactatt |
| 40081 | tttagatatg acgtatcaga gtgatcaata ctgtgaagcc tcttacaaca gtgtttagat |
| 40141 | tgaaaaatta tgagtgaaaa gcctttcaca gtcattcgga ttgaatatgc ctaacatgaa |
| 40201 | atccaaactg tttcaacatt tctttcaagt cattttctac tatggaaata ataatgcatg |
| 40261 | atttagccat gcctgagttt tatttcctca cgcaggcatt tatttcttta aaaaatttaa |
| 40321 | tttaaagttc actataaaac ttaccatttt aaccatttga agtgtacact tcagtggcat |
| 40381 | taagtacatt cacaatgttg tgcaaccatc actaccatcc atctccagga cttctttatc |
| 40441 | atcccaaaca taaaccatat ctactaaaca gtaactcccc aattccttct ccagcccctg |
| 40501 | gtaacctcat tttatatgcc atcacaatat tttagaataa acgccaactt cctggggtaa |
| 40561 | gatcatttga cttttgctcc cagatcagaa ctatgataaa aatattattt gctatatact |
| 40621 | aacagggaaa tttctggatt ttggcatgag ttactgtatt ataactctaa atcctggtaa |
| 40681 | gggaagcatt tttattctga aagtaaacaa aaataaccat tagtatgata atctttacct |
| 40741 | ctgggggtgg ggtattttgg agggggtgag tacaaagaga ggtgaaatta ttcctctagg |
| 40801 | gtggcagacc agagtttaaa ttgtttctta Kgtttttcat cttggttgtc taccaattgg |
| 40861 | atatagtcac tcagatcatc catgtttttg tacctggttt gataggtata aatacctata |
| 40921 | aattggaata aattctagca attagaggtc catgttcctt ggtctgtaaa gtgaatataa |
| 40981 | caaaactgac ttctctccaa agggtgcttt gagacaaaac tataaaacac tttggagagt |
| 41041 | agaagggggg tccttatttt tagtaacatt cctcagtgtt cttcaagaac tcacctactg |
| 41101 | ccaatcattc ctctgagtta agaatatata ttagaactag ttttaatagc actataaact |
| 41161 | atagatagca tggccatatg tccctgtttg cctggagctg aataaattac aggttcaaaa |
| 41221 | atactcaaat aataaaattg cctaggtggt gacccaccta gtttgttgct gtatggtgtt |
| 41281 | gggtatctct tttttttcct tgacaaattt gaaaaggcct tgaaaccatg agcagattcc |
| 41341 | ttggttcaga tggtctgtaa taggagacac caggacaagc ctgagtctcc tgctcttgaa |
| 41401 | agattagcat attgatagat tgcRtaccca ttatcagtta ggtatctgcc aaaccatttg |
| 41461 | cagcgtttct ctggagttca tacagttggg aagacttcct agaggcaaga ggtcttttac |
| 41521 | ctgaagattc agcagttact ataggggcct tcttttggct tcttgctctt gggacacctt |
| 41581 | tacttgaatg aggtagtctt ttgccattag tgcctgtggg gccttaatga ttcctattgg |
| 41641 | gatgagttgg ccagaagtga ggctctccac ttcatgttga aactaacttc tgcctgagta |
| 41701 | ttaaggatgg gtgattctga ggtcctgagc cagctggaag gactttcatt cactttctac |
| 41761 | tcttgtctga agaatgctta ggctacctta ctgttacttc tctggtggta ccaaaggcca |
| 41821 | tgtaatgaca tggctggatg cgatcactca cgtctgtaat cccagcactt tgggaggcca |
| 41881 | aggcaggagg atcacttSag cccaggagtt caaccagcct gggcgacata ggaagacccc |
| 41941 | catctctaaa aaatttttaa aaaatctagc tgggcatgtt ggtRtactcc tgtagtccca |
| 42001 | gctactcggc aggctgagat gggaggattg cttgagctta ggcgattgag gctgcggtga |
| 42061 | accatgatca caccactgca ctccagcttg ggcaacagag caagaccctg tctctaaaaa |
| 42121 | aaaaaaaaaa aaacaaacaa aagatgtgat gattgcctct ttcggttata tcctgctgcc |
| 42181 | taagagccca ccccagaatt tagtggctta ggataaccat tttcatagtt ttgtcagtga |
| 42241 | gggattaggg aagatgtcag ctgagctctt catttctgct ccagatggca ttggttgggc |
| 42301 | aatgggactg gaggttctaa ttctaagata gcttcttcac tcttatgaat agctcctcag |
| 42361 | tgtctgtggc cagtctctct ccatgtggta tctcatccac cctggccttt ccacatgcct |
| 42421 | taggcttccc acatcatggt ggttcctggg tagttactct tcttacagag tagggatgaa |
| 42481 | atgaaagcct ctattctaaa ttcagttaaa tactggtaca aatatttagt actgggattg |
| 42541 | tgggatcatt ttcttatatc ataattataa cccaatcagg gtagaaaagt gaaagaaaga |
| 42601 | aggacacttc attttctttg ctctttgtgt ggatgatggg ttgtacaaat agcaggaggt |
| 42661 | tcaacttttg gcttgttttg gattactgag tactatcctt cagaaatgat ttcctaatac |
| 42721 | tttcccggcc atctggcatg agcacagaag accttttact caataaaagt aagctaataa |
| 42781 | taataagttg aaacatcttg ataataaata tatttgtaca gcttttcaaa tcccaggtta |
| 42841 | ttgaaattct gtcagccttg gctggtttcc atgctccttc tgtgcccttg tgtttccttt |
| 42901 | ttttcttttt ttttttgttg agccactcct gctgacttcc aaaggatgct ctgagctcta |
| 42961 | aaaggaggag cagttttgct ggcatgcaga agtggtgaag gtaaggcttg gattcaatta |
| 43021 | cagtaatctc agaagttctt ggtagaagag gggcacagaa tcccagcctc cattttgcag |
| 43081 | gtaaggaaga agaattataa aaagataaaa tggcttttct taatgtcagc atctgattct |
| 43141 | ttaccttctt tcaccctagc aaagattgtt aacataaaac agatgttcct tggacaagag |
| 43201 | aaaagtaata gagctggatc ttttcatgat cctgcattta ttatttgtag cactggatct |
| 43261 | ctaaactagg aaaagcaatg tcgattctaa ctagaaatta tggggcattt aataacacgt |
| 43321 | gaggcaaaat tatgaggaaa ggcaagaaac caagtggctc ctagcttcta acaccttatt |
| 43381 | ttctatggtg taagttaatg aatgatccat ttttaatgcc ataaaaaagg aaatttccga |
| 43441 | taggctttac cttaacttca cgttgctgtt tatattacag acaaaatgct aaagttaaca |
| 43501 | agtgtaaaat gccctaggtc agggtttcga gtgcccataa gcaattctct agaattattt |
| 43561 | tgagtgctca gattatagat ttgcccccat accatatact gtattaccta tcactgcaag |
| 43621 | attctttata gggtgtcaag tatgtgggag gatttaaact aattgtctta aatgcttcat |
| 43681 | gggataggag atgatccaat gggaactgaa tgttttcaat taggatgttt ccatggatac |
| 43741 | tattaagcat gccaagattc atagccacag gagattttta cttacttcaa tattgattaa |
| 43801 | gaaatctgag aacaatctca agaagtgtag gatgtaggcg gaggataaat tttgaggata |
| 43861 | gtctaaaatt tgctttctct tccatagtta aggcctagtc cagaaaaagt ctaggcagtc |
| 43921 | agccaagtct aacagcagga tttatgtcaa gagaatgtcc agaataaaag tgaacatggg |
| 43981 | ttgtttcatt taccccaggt gtatttatta acatttattt aatgttactt attatttgtt |
| 44041 | tattaccatt agctttctta tgtactcggt acaaagcatt agaattacaa ccctttgtat |
| 44101 | ctttgttgtt tatgaggaaa acatcctaaa tagtgtctac agacatctta aaattcacat |
| 44161 | tcctctgaga aaggtttatc ccttaggtta ttactttttt ttttttttcc acacatgggt |
| 44221 | tttttacctg ggttctcaga tctccaagaa atcggtggat agaattcaag gtgacttctt |
| 44281 | ggatggagaa aaagtacgtc tttattttga ccaaccatgg atatttagca ttttcatcaa |
| 44341 | ttaatgaatg caggtgacaa atcacagtac tattggcccc atctttgagt ttgtcctcaa |
| 44401 | tagctcacag atattttcat attgtatcac agttgttgca gatatcttga aatattgttt |
| 44461 | attctaatca ctccttcaaa atttgggtgg ttattggcat tatcaccagt tttttttttt |
| 44521 | tttttgaggc agtctttctc tgttaccccc aggctggagt gcagtggtgt gatcttggct |
| 44581 | cactgtgacc tctgcctccc aggttcaagt gattctcccg cctcagcttc ctgagtagct |
| 44641 | gggattacag gcacatgcca ccacggcccg gctaatttat ttatttattt attctttatt |
| 44701 | ttttattttt aaattttttt tagatggagt ctcactctgt cgccaggctg gagtgcagtg |
| 44761 | gtgtgatctt ggctcactgc aacctccgcc tcccaggttc aagcgattct cctgcctcag |
| 44821 | cctcccgagt agttgggact acgggagcgt gccaccacgc ccagctaatt tttctatttt |
| 44881 | aagtagagat ggagttttac catgttggcc aggatggtcg cgatctcttg acctcgtgat |
| 44941 | ccacctgcct cggccaaaat gctgggatta caggcgtgag ccaccgtgcc tggccacatt |
| 45001 | accaccagtt cttgatactt aatgccataa caaaaggcac atgtatcact atatcacaaa |
| 45061 | ttagcatttt ataatatttt aataacgttt tcaatataag tggcttccct tgtaatcata |
| 45121 | tattttattt ttgcacttaa aaatataatt cctaaaaggg atccataaag cttcaccaga |
| 45181 | ctgccaaagc gtccatggca caggacgtta agaaccctgt tccaaatgga atgaagatta |
| 45241 | gaagcactat catatgttca ttttctgatc gcagtttaag aaagagtttc ctacttaagt |
| 45301 | gcttttcaca gaaaatctaa acttaaaggt taaaagggaa ttcttttgtg ctggctcatg |
| 45361 | tgtgggccct acaaagtact ttgcagtcat atctgacttt cgtttgagag tttgtcttca |
| 45421 | gaaatggaag aagaaaaggc attcaggtat atgacaggtt gcattctgta gaacattgtt |
| 45481 | aaatctatgg gaggggagaa aagttccatt cagtgtcttg tggattgtgg taggagtcag |
| 45541 | ttgtgttctt gcagaaattt ccctagacag tgcctcttgc ctcctccttt cttgtcccac |
| 45601 | agtactccat ggagcagatg tgggaacttg gatgcctgat gggaattgta accccatgct |
| 45661 | ttgccttatc ctggtgggga ctgatctgag gtttatccat taaactgtat cagttcatgg |
| 45721 | ctgggagtag gaatgcagaa aacacaagca aaatgactgc aggaaggttt aaataaaatc |
| 45781 | tcatatgtgg cttttcctta acacattatt ttgcaaaaag tggcagagtg aatttgctgt |
| 45841 | gatatgatag gatcattctg gacttttact taatttacgt gttctctagc agaatgctga |
| 45901 | aggagcacac ataaggagtg gggagttggt atttggttct gtgacaagtg ctatcttata |
| 45961 | acagaaaaat acatattttt gtgaaacttc aaatgcattt ttattcccca gcacatctgt |
| 46021 | gattctggat ccactacctt cttccaagtg tgatcaagca ctgtacctga cagtatcgat |
| 46081 | tatccttacc cgtaggcaaa aattgtatta catttatttt acagtgatct ggaaaaaaaa |
| 46141 | ggatgacaac aaaaaccaga cacacttcat ttctattccg gtaacatttt ttggtaataa |
| 46201 | tttattactg tacaagtcac acaccatata agtcacacat ttaaagtgtc aaattaaatg |
| 46261 | gtttttagta tattcacagg atcgtgccat catcacagtc aaatttagaa cacttattac |
| 46321 | ccccaacaag aaacctcata cgctttagca gtcaccctcc attgcccccc agttttctaa |
| 46381 | ccccaggccc taacaaccac taacctactt tctgtctcta tgaatttgcc tcttctggcc |
| 46441 | atttcatata aatggaatta cacattttgt aaccttttgt gactggcttt tttcacttag |
| 46501 | cataatgttt tttaaggtct acctatatta gagtatgtat cagtacttta ttccttttta |
| 46561 | tggccaaata atattccatt ttctggatat accagatttt gtttatccat tcatctgttg |
| 46621 | atggacattt gagttgtttc cactttgtgc tattattatg aataatgctg ctatgaacat |
| 46681 | tcatgtttac atcatgtata cattttaatg tggacatatg tttttagtta tcttgagtat |
| 46741 | atacctagga gtggaatcat atatatatgt ttttaaacca aaatcaaatt ttaatgttgc |
| 46801 | agaatccctg aagcactttt aggaactgtt cacttccata gaacagtttg caaatcagtg |
| 46861 | ttaaagtgga gagagtgttg atttttttgt gcatgtattt ggtagacatc gtttgaatct |
| 46921 | tgattttata ggctgtgtga attttgtcaa gttacctgtg aacatctttc ttctagtata |
| 46981 | gaatgttagt acctctggat gtttcacgag ttgatagact gtattattgc ttgaaaaatt |
| 47041 | cactgtccat ttattaccat caactttggc cacggatttg ctttggctag tggcctgtga |
| 47101 | ggggaagtga tgtgtgcctt cttccaggca gaagctggtg gtttgcctca tctcttttcc |
| 47161 | tttctgccat gatgaccatt gatgttccag ttggacacag gctccatcag cttgtgtatc |
| 47221 | agaatgaaaa tgatgtgggg cagagccctc atagattttg ttgcatgagt atggaaaaag |
| 47281 | tgtcatttta agccgtcaag atttggtgac tatttgatac tttagcacag tggtcctcaa |
| 47341 | gcaatggcag ttttgcattt ctcacactga gatatctggc aatgtctgga gacatttttt |
| 47401 | ggttatcaca catggggaag tgctcttggc atttagtggg cagatgctgc taagcatcca |
| 47461 | acactgcaca ggacagctcc cacaacaaag aattatctga tccaaaatgt caatagtgct |
| 47521 | gaagtttaga aaccctgctt tagtataacc tagtttatcc tggttaagat agggtgttga |
| 47581 | gaagattaaa tggaaaaaaa atatataaaa tacagcacac agggtggtct ttttttggta |
| 47641 | attatgtgac agagcttggc aatggctctg aggtcttccc ttgcatttgt gggtcttttc |
| 47701 | caacttccct agggttcagt attcagttta attgtagttg tgtttataaa tcaaggctcg |
| 47761 | tttttagcag aatcatatac cctttaaaaa agggggagaa ctaacattag atgcctgttt |
| 47821 | ggcaccaggc tctttgtatg tgttttgaat ttttacagat attctctgaa actgatattc |
| 47881 | cttacctcca ttttgttttg ttttgttttg ttttgttttg agacaggatc tcactgtgtt |
| 47941 | gcccagactg gagtgcagtg gtgcgatctt gtctcactgc aacctccacc tcctgaattc |
| 48001 | aagcaattct cctgcctcag cctcctgagt agctgggatt acaggcatgc gccaccatgc |
| 48061 | ccagttaatg ttttgtattt tcagtagaga tgggatttcg ctatgttggc caggctggtc |
| 48121 | tcttaacttt tggcctcctg tgatccccca gccttggcct cccaaagtgc tgggattaca |
| 48181 | ggcatgagcc accatgcccg gccctccatt ttgtttttga tgaagacatt gaggcactga |
| 48241 | agttaagtaa tttgaccaag atcacatggc ttataagtgg tgggcctaga attcgaagca |
| 48301 | catctgtttg cctccagaac cctcttcttt ccacactgct atactacctc tttagcagtg |
| 48361 | actttaacat agagaaatga aggtaatacc tgatttctaa caattatgtg ttgtattagt |
| 48421 | tatctattgc tgtataacag gttacctcaa aacttagcac tttaacacaa caaacagtta |
| 48481 | ttgtctgaca tagttttgag aatcagtaat ttaggggaag ctaagctggg tggttctggc |
| 48541 | tcaggatctc tatggagatt gcaaacaagt tgtcttccag ggctgcagcg atctgaagac |
| 48601 | ctgattggag caggaagatc cacttccatt gtggtgcact cacagggctg ttggtgggag |
| 48661 | gcctcagttc ctcaccatgt gggtctctct ttagggttgc ttgattgtca tcatgacatg |
| 48721 | gcagctggcc tccttcagag tgagggatct cagagagaca gcaaggcaga agccacaata |
| 48781 | tcttttgtga cctagcctta gaagtcacag atgatcactt ctgctctttt ctattggtca |
| 48841 | catacacaac cctgatataa tgtgggagag gatgatacaa gggcatcaag aatactatga |
| 48901 | gatggaaatc actgggggcc atcatggagg ctggttacta catgttaatg atcctcattt |
| 48961 | gccatagtta caaaattatg aattatattt ccttttcata ttctaatatt ccaaacacct |
| 49021 | gttttgtact ttaagatact taaatcattt agaatgtgag tgaaaataca aattggcttt |
| 49081 | tgtaaacact tactgaatgg gtaaaataaa ggccgtggta cagatacttc attggggaat |
| 49141 | ttttttttta aataggtttg actagctgag gcttcttgaa tcctgaaaag tttggtctga |
| 49201 | tgtacttttt tttttttcct ggaaaatctc cttgtaccaa acactgaatt cttcatttgc |
| 49261 | caaagtccac ccaactttct gatgttttta ttgctggttt gatgtgcttg aatgccagcc |
| 49321 | agagtataag gtgaatggta gatgcaagga ctgtgcagtt tccacggaga cttctttcag |
| 49381 | gagcccttca ttttttcatt cagctattgc tgggaggcat ccccttccag ttaatgtgtg |
| 49441 | gtacacatca atttgagtgg ctcttagaaa ggggctcagt ttatacaaat taagaattga |
| 49501 | ggtttttttt ttctctgtat atcttcaacg ttaaattaga tttctatgaa acttgaagat |
| 49561 | gttaacagga aatcttcctg aaacagttta actatacttt tgtttattac acttgatttt |
| 49621 | gagcttttgg gaataaaagt agggtcctgg ctaggtacag aggtatatgg atttactgtg |
| 49681 | tctaatcgtg caaccatgaa ataaacgttt gcacttcagg aatcaaccca tcacttttta |
| 49741 | accactgttg tttgcattgg cttagagctt tgtgcttcct tcaactattt tccttttttc |
| 49801 | tttcaaaact gtgttcagtt ggttgtgacc aattttacac agagactggc tcactcactt |
| 49861 | atgttcttga ctcctgctgt cccaatatca tgcttcattt acacagggat agcttttcct |
| 49921 | ggctttatct gttgacttga catcctgtgc atcatatttc ttttcttaga tatagagaag |
| 49981 | actgaaaaaa atccacgatt tgaagaaaat ttRcaaaagt gcagagcaat gtgaaagaga |
| 50041 | tagctttaat tttaattttc actgtagtca gaataggtgg cctcctgatg ggcagacttg |
| 50101 | ctttctcatt ttactctgtc cagtgtagat acccagatca atcaaggtca agctggtctg |
| 50161 | gtcattaata caacttttgg ttttaaacac agctgtgtac gggggttctt agcattatta |
| 50221 | atagaggtta taatgaaaac aaggtatagt catacagttg tctgtgtggg tagtggcatg |
| 50281 | gaggtggagt tgttgcttaa gatctgaaag tcaggaagct ggaggttttt gtaattttat |
| 50341 | tcattatcaa tcttaagaat ttattttgat cattataaat gcatgactgg tgctattttc |
| 50401 | caatcctgag gttttttggg aaattttttt gaaaagaggc caggatgagg ataaaaatta |
| 50461 | attttgaaaa tccatttcaa agactcatta tgctatgctc catcactgca attttaatgc |
| 50521 | aacttatata ttgtgctttt acctttggga ctagcattct taaaagctga cttttaattt |
| 50581 | ggactttcta atatgtgtcg gctctgtcag ctcacgagtg tgtttttatc tStgcatcat |
| 50641 | gttatattgg gacctgggtg gaatgtaaat cttttactgg cctaaatctg agtctaatcc |
| 50701 | tctgcatggc tttggtagaa aatcaatttc ctctgctctg tcaagatttt tcaggtcagt |
| 50761 | gctctttgga agtttcccag ttctcttaga ttaaaataga ttttgtttct gtgctacata |
| 50821 | ctggtattag ttagtaggac actgggatct aaaagcaaga gaacagaggc agacaagata |
| 50881 | tccatgagtt cttctatttc agttaacttg ttgacacact gcacctttga ggaaccaagg |
| 50941 | agtttgtgtg ctttcaggca tcatttgggg gagcaattgt aaatgactcc tgtaggtgag |
| 51001 | gctgtgcact taattttaat gagttcaggt tcattaagtg acctgctcac tgggtcatta |
| 51061 | gaagtgacct cactagaagt gaatggtaaa gagaagaact acctaagttc cactcagctt |
| 51121 | gtttcactac tgtggggtat tggaagtaga gtgacaactt ggactttctc agttttagca |
| 51181 | ctgaaaagtc ccacatccca ggaacttact cagtccccag caaactaggg ggagcggggg |
| 51241 | atggttggtc cccctagcag tgaaaagtaa ggggatggat gatttggaac tggttagtac |
| 51301 | ccttatagat tttttttttt ttttctgaga caggttcttg ctgtgtcacc caggctagaa |
| 51361 | tatagtggca tgatcatggc tcactgcagc cacgatctcc tgggtccaaa tgatactccc |
| 51421 | acttcagcct ccccagtagc tggaactacg gggacaagcc accacaccca gctaattttt |
| 51481 | atttatttat attaattaat taatttattt ttttgtaggg acagggtctc gctttgttgc |
| 51541 | ccaggctggt cttgaactcc tgggctcaag tgatcctcct gccttagcct ctcaaagcac |
| 51601 | tgggattaca ggtgtaaacc accatgcctg gctgtcttat aaatacttta aggagggatt |
| 51661 | tattagagac atgtagcacc ttgctgccca gtgatgctag taaatgagtg actctacttt |
| 51721 | gatttgacag aagaaacaat ggaaagagga gagatggaag tttttctacc taaacttcct |
| 51781 | aagaggatgt gtttagggga tgtgttggta aaaagttatt agcattaatg tttacctgta |
| 51841 | tctttctcat agataatgca aagaccttta actccactta tccctccttt taacttatgt |
| 51901 | tgttattgcc ttgtatttta atactatata ttaaactcta caagacatca ttattttatg |
| 51961 | caggcattgt ttatttaatc agagttcagt aaactttttc tgtaaatgcc agatggtaaa |
| 52021 | tattttaacc cctgcaggcc ttatagtctc tgtcacaact actcaactcc actcttaata |
| 52081 | gtgccaaaat ggctatagac aatatggaaa tgattgtgtg tacccatgtt ccaataaaat |
| 52141 | ttatttacag aaacagacag tgggccagat ttggcctgca ggccttactt tgacaactcc |
| 52201 | taatttagat ttatctacat atttatttat tttcccttta tttcttgcta tatctttgag |
| 52261 | gttctacctg ggatcatttt caaggcaagg atgtctattc tcatcactgt ttttcagcgt |
| 52321 | tgtgctagaa attccaacca gtgcaataag gcaagaaaag gaaataaaag gcatacagag |
| 52381 | cagaaaggaa aaaaaaaatg aaactggcca ggtacagctg ctcatacttt gagaggcgag |
| 52441 | ctaggaggat tgcttgagcc caggagttcc agaccagctt gggcaatgta gggcgacccc |
| 52501 | catctctaca aaagtaaaaa taaaaaaaaa attagcaggg tgttatggtg catgtctgtg |
| 52561 | gtctcagcta cttgggaggc tgaggtggga ggatcacttg ggccttggag gtgaagtgag |
| 52621 | ccatggtgac tgtatcactg cattctagcc tgggcaacag agtgacaccg cctgtctcag |
| 52681 | aaaacaaaac aaaaacctgt ctctatttgc agatgacatg attgtctaca tagaagaccc |
| 52741 | tgagaaatct acagtctaca gaataactcc tagacctaat taagtgagtt cagcaaggtt |
| 52801 | tcaggatata agataaacat acaaaaatga gttgtatttc tacatactag ccataaacaa |
| 52861 | atggacactg aaattaaaaa tacaatacca tttacattca ctcaaaaaat acttgggtat |
| 52921 | aaatctaaca aagcatgcac aggacttgta tgctgaaaac tatacaatgc tgatgaaaga |
| 52981 | aatcaaagtc taaatagatg gggaaacata ccatgttccg ggtttcagga tataagataa |
| 53041 | acttacaaaa atgagttgta tttcacatac tagctagaaa caaatggaca ctgaaattaa |
| 53101 | aaatacaata ccatttacat tcactcaaga aatacttggg tataaatcta acaaagcatg |
| 53161 | cacaggactt gtatgctgaa aactacaaaa tgctgatgaa agaaatcaaa gtctaaatag |
| 53221 | atggggaaca taccatgttc caggattgga agactcaaca tagtaaagat atcatttctc |
| 53281 | ccccaaattg atatacaggt ttaatgtaat tcctgtcaaa atcttagtaa gatttttttg |
| 53341 | tagatataat tataaaattt atatggaaag ggaaaagaac tagactagct gaaacaattt |
| 53401 | tgaaaaagaa taataaagca ggaggaatca gtctgtaaga ttttaagaca tataactaca |
| 53461 | gtaatcaaga ctgtgtgata ttggtggagg gatacatgta tagatcaacg gaacagaaca |
| 53521 | aagaacctag aaatagacca acagaaatgt gcccaattga tctttgacaa aattgcaaca |
| 53581 | gcaattgaac ggagaaggaa agatagcctt tttgacaaat ggtgctaggt caattaaaca |
| 53641 | tcaatatgtc aaaaaaaaaa aaaaactctt gctgtaaata gtacacgtta tatgaaaaat |
| 53701 | aactccaaat gtatcacaaa cttcaatgta aaacataaaa ctataaaaat aattagagaa |
| 53761 | aatcttcagg atctaaggct aggcagagtt cttagacctg acaccaaagg tatgatccat |
| 53821 | aaaagaattg ataaattgga tttcatcaaa ataaaaaata cttactctga gaaagacctc |
| 53881 | ataagaggat gaaaagacag actacagagt ggggggaaat atttgcaaac tacgtatccg |
| 53941 | acaaaggact ggcatctaga atatataaat aactttcaaa acttaacagt agaaaacaac |
| 54001 | ccagttagaa agtggaaaaa atacaagaag ggacatttca ctgaagagca tatacagatg |
| 54061 | acagataagc atgtgaaaag atgttcaaca taattagcca ctagggaaat gtaaattcag |
| 54121 | accaaagtga gctatcacta catacctata agaatggcta aaattaaaaa tagtgacagc |
| 54181 | atcaaatgat ggcaaggctg tagagaaatg gaatcattca tactttgctg atgggatgta |
| 54241 | aaatgataca gcccctctga aaaacagttt ggcagtttct taaagacact aaacatgcaa |
| 54301 | ctaccatatg acccagcaat tgcactcctg ggcatttatc ccagagaagt gaagatttat |
| 54361 | gttcatataa atgccagtat gtgaatgttt tataggagtt ttattcataa tagcccataa |
| 54421 | ctggaaacaa tacagatgtt ctttagtgga tgaatggtta gacaaactgg tatatccata |
| 54481 | gcatggaata Ytacccagca gtaaaaagga gtgaagtact gatacatgca atgatctgga |
| 54541 | tggatctcca gagaattatg ccgagtgaga aaagccaatc ctgagtggtt taaataccat |
| 54601 | atgattccat ttatgtaaca ttcttgaagt gacaaaatta tggagatgga gaacagatta |
| 54661 | gtccatgcca ggggttgagg gggcaagggt tgggagtaaa atggatagct ataaaaggca |
| 54721 | tgatgaggcc aggcgtggtg gctcacgcct gtaattccag cactttaaga ggccgaggcg |
| 54781 | ggtgaattgc ttgaggtcag gagtttgaga tcagcctgac caacatggtg aaaccctgtt |
| 54841 | tttactaaaa atacaaaaag tagctgggtg tagtggcagg tgcctgtaat cccagctact |
| 54901 | ccagaggctg aggcaggaga atcgcttgaa cctgggaggc agaggttgta gtgagctgag |
| 54961 | attgtgccac tgaactccag cctgggcaac agagcaagac tctgtcttag gaaaaaaaaa |
| 55021 | aaaaggcatc atgggtcctt gtgagcatga aaatgttctc tattgactgt gtcaatagag |
| 55081 | acacacaatt atgttgtgtg tcacaaccat aatcacaaca ttatggttgt gatattgtac |
| 55141 | tgtagtttta caagatgtta ccattgggag gaagtgggca aagggtacat aggatctctg |
| 55201 | ggtatcattt cttacctctg catataaaac tacagttgtc tcaaaaggtt taattaagaa |
| 55261 | agtatttaag aaaaagaacc ccaggatcta aaaagatcag agtcagagaa tcacaaatgc |
| 55321 | taaataagaa gcacttttgt atttggtgat gttctgaaat tctggctcag ttttgttatg |
| 55381 | aaatggagcc acgtgggtcc atgtgagtga gtccctgtgc aYaggtgtat gattgacttt |
| 55441 | ttccctcaag tgcttctaat ttgggatgtt cttcactgtg tgccctttga gtgtctcttg |
| 55501 | cctctggttt tttgttagac aaaaaaacca gtttttaaat gtcagttcca aggtcttttc |
| 55561 | tgggtacggt taacttttat gatttcattt atgcagtggg ttttcaccta agggccatgg |
| 55621 | tttctagaca ctagactcgt ttttcccaag cgattcatct ctttgcctgt gggatggaga |
| 55681 | gattcattct cttcttctct tttgcagtag aaactccagt tttgagctgg gcacctggat |
| 55741 | gcccagagta aaggttgtac tgcccacatt tcattgtagc taggtgtgcc tgcgtgacca |
| 55801 | agtatgaacc ttctatttag attaagccac aagataagtg gagctatcaa ttggctttaa |
| 55861 | caaatattta gtgaataggt aaaataaaat actttatata agagctgtgt gttctgggtt |
| 55921 | gaattgtgtc ccccacaaaa agatatatta aagtcgtaac ccttagtacc tcagaatgtg |
| 55981 | accttatttg gaaagagggt ctttgcagat ataattaatt tcaggtgagg ttataccgga |
| 56041 | gtagggtggg ctcctaatcc aatatgatga gtgtcctcag aagaaggcag tcaggcacat |
| 56101 | gtaccctaaa acttaaagta gaataaaaaa taaataaata aataaataaa taaaaaagaa |
| 56161 | ggcagtcagg tgaagacaca gacacacagt gggaaccccc tatgaagatg aagatagaag |
| 56221 | caatgtatct acaagccagg gaccgcctgc ggctgcaaga agccaggtga gacacatgga |
| 56281 | gctgattctc cctcacagcc cttagaagga acgtgccctg ctgacacctt ggttttggac |
| 56341 | ttccagtctc cagaaccatg ggataataaa tacatttctg ttgtttgaag ccacccaatt |
| 56401 | tgtggtactt tattatagca gccctaggaa accaatatac tatgcttttg atattatttt |
| 56461 | taggatgttg tggtttgaat atttgtgtct cccccaaatt cctatgttga aacctagtct |
| 56521 | ctgatgcgat agtattaaga ggtggggcct ttaggaggtg catgagggca gagcccttat |
| 56581 | gatgggatta gtgttcctag aaaagagact tgagggagct tgtttgcccc ttccagtatg |
| 56641 | tgaggataca gcaagaaagt gccatctatg aggcagagag tgagccctca ctatacactg |
| 56701 | aatctgctgg caccttgatc ttagacttcc cagcctccag aatggaaaga aatttctgtt |
| 56761 | gtttataaat tactgagtct aaggtatttt gttgtagcag ccccaacaaa ctaagacatg |
| 56821 | gagtatttta aaaaatgggt tgaacattca gaggcttctt aatctgagtc cttagatatt |
| 56881 | tggactgact ctgccttctt tttccagtga aaagatctcg ctgtatgaaa catagacatt |
| 56941 | cttcattgcc ccaaagccct tccaccttcc ttatgttttt attgctggtt tgatgtgctt |
| 57001 | gaatgctagc caaagtataa ggtgaatggt agatgcaagg actgggcggt ttccatggag |
| 57061 | acttcttgca ggagcctttc ctttctcagt tcaacagttg tcaaggaaac atctagtttc |
| 57121 | aattaaataa atgtttgtgg ggtctgccaa tttgagtgtc tcttggaagg ggatttggtt |
| 57181 | aaacttctta aacatcgtaa taaacaaatt cagagcagag gctttttagg ttcccttcca |
| 57241 | gttatattag gtttttctgt aacttaggga tgttaactag tcctctgtct gaagtgatca |
| 57301 | gactatttat tcatatccag aaaatattat ttttgagtct tgtggggagg aaaactgagg |
| 57361 | cttaaagaaa gagaggccaa agccagggag ctggtgttac atggcaccag ctatatatac |
| 57421 | atgactatag tccatgctct ttagtacgtc ctgcaggata agagatccaa ggggcctgac |
| 57481 | ctttacaaac tgtttacagg ctatggagtt tgctggctca gcactgtgtg tttattgctt |
| 57541 | ctagttagct attgttgtat cagtggctgg agttggtgat ggttcacgga aatagtggtg |
| 57601 | agagtacaga ttctaggtat atgcaattct aataaatgag acttccctag tgttatctgt |
| 57661 | attgtgacag cctccccatt gtgctcttta tcaaacagta aggtatggta tattgcaaaa |
| 57721 | tgctcacaat actttgcaac tcttcccatc aagaggtaga gtcagtttct ctattcttag |
| 57781 | gatcttggct gtcctgttga tttggtctga ccctggaata cagtagaaaa gaccctgtgt |
| 57841 | gggttttgat cctaggcgtg aggtagagct tcagtttctt gctgctttgg gaactctgcc |
| 57901 | accttgtaaa aaaagccctg gctggcctcc tggaggaaga gagactatgt ggaggaaaat |
| 57961 | gaaggtaccc agacaaccca atagtcaact gcagaagcat aagtgaccct catctgagat |
| 58021 | caatggtgcc tggctcggat cagtataatt gtccagctga actcagccca aattactgac |
| 58081 | ccccagagtt atgaattaaa taaatgctgt tgctttaagg cacttaagtt ttggggtggt |
| 58141 | gtgttataca gtagtagata attgatacag ccaagtttgg aagctctgga atgatcaggg |
| 58201 | ctcacctttg acctgtcctc tataaaaccc aggattctta gtaacacagt cctgttaata |
| 58261 | acagaacttg cctatggcaa atctaagcgg aaaagcagtt tattggcagg atattgtgta |
| 58321 | gctacatctt tctttaggaa gaccagggac aaggcttgaa aactgcacaa ctagggggca |
| 58381 | gtactgccag aactacagcc agataacctc acagaattgg tagctactga atattggatg |
| 58441 | cctccaccta caccaccaac attgcctgca ttggacattg ggtatggggc tgctcctgga |
| 58501 | actggatgtt gttcctgtca ctagaaaaca atcatctact gttcttgctg tttcacagca |
| 58561 | ctcattactg attcagatgc ccgtgtgcca cgtgtttata gcagtgcaat ttgtaattgc |
| 58621 | aaaaataggg aaccagccca aatgcccatc aatcgagtgg ataaagaaaa tatatcaaaa |
| 58681 | atatatttta tatgtgtgtg tgtatgtata tgtgtgtata tatgtgtgta tgtatatgtg |
| 58741 | tgtatatata cacacacgta catatatata catatatata cacacacgta catatatata |
| 58801 | cacacacgta catatatatg cacacacaca tatatataca cacacgtaac atttataata |
| 58861 | cgccatggaa tactactcag ccataaaaag gaatgaaata atagcatttg caacaacgtg |
| 58921 | gatggagttg gagaccatta ttctaagtga agtgactcag gaatggaaaa ccgaacgttg |
| 58981 | tattttctca cttataagtg ggagctaagc tatgaggatg caaaggcata agaatgaaac |
| 59041 | agtggacttt ggggcctcag agggaagggt gggaggtgag ggataaaaga ctacacattg |
| 59101 | ggtacagtgt acactgctcg gctgatgagt acctcaaaat ctcagaaatc agcactgaag |
| 59161 | aacttaactc atgtaaccaa ataccatctg ttcccccaaa gctatttaaa taataataaa |
| 59221 | aacaaaaaca aatgcctgtg tgggtggtgt ctggcagagc gtaggtcttg tgcccatacc |
| 59281 | ctgattgaaa agaaatcagg aaagtgagaa tctgacactt ttagcttatt tagctgaaag |
| 59341 | caggtggaaa atttcataaa cactggcaaa gggttcggat aacaggcagc caaaagaaaa |
| 59401 | atggcaaatg ttcattagtt tctgttgtat actctggtta gctcagttgg ttacagcaca |
| 59461 | gttatgatga agccagcatt gtggtttgtt cttaagtttt attgaaagaa aatagttctg |
| 59521 | agattgtccc ccttacacct gctccctctc tcacagatgt gtgctgatag ccatttgtaa |
| 59581 | ggccaggcaa tagtggggcc taacttgcat ctgtctctgg ctgctaaggt acaaagatta |
| 59641 | tgtgtctttc agttggtggg cccagagact ccattcactg gcatagtgtg ccacataaga |
| 59701 | gaactgaatg aatggtatct gtgtgaatct aaggggttgg ctaattgcgc agtgaatttg |
| 59761 | gttcatgtaa catttataat acaaattgtt gggcagcaag atctttcaat ccagacggtt |
| 59821 | tctagttttt cagaatttct ctgataactt ctctgaactc tgaaattctt atttgcaaaa |
| 59881 | ccatctatat agggttcatt taaatattag aaatagtact cataaagaga aaagcataat |
| 59941 | gcctagagga tgtgtaatag atgttcaata aatgttgatc atttccctca aacttcttag |
| 60001 | attcacacac cccaaccaga ttagaaactc tgacacggct tgcccttttt gtgttcaatt |
| 60061 | ctctctcagt tgttaagtga agacccaatc cagttgttaa aataataaat gcacctcttg |
| 60121 | cctccctgac attctggcaa ccttactctg aggaggctgg cccggttcga agctggctgc |
| 60181 | tccagggcac taaagctgga gaagggccct tccaccctgt tcataactgt ggacagtaaa |
| 60241 | ctgtatttgg gatagaggat cttgggaaca ataaggacct agatccaagc tcatgcattg |
| 60301 | tggtgggact gtcaatttag ggggatgggt agattttatc agatctgtca gtggaaagtt |
| 60361 | tggggagatg gaatagtatc tacaggtagt tccaggtagg aggaagagat ctacctggct |
| 60421 | cctagggatg tccatcctgg caagcattgc tcagtggcag gagttgtaag agtagggaag |
| 60481 | gtttaggaat ttggatatca gggaagtgtc agcaggtagc tggggatgag gtgcaggaca |
| 60541 | aagacagacg agaggaagac caagccttaa agtgatgagt atctagatag ggaagaggat |
| 60601 | ggatgggtat agcttggaca cagaaagggg attcagtcat ggaaagaggt cagtctgcaa |
| 60661 | tgtaatgtgg ctatcaggtc acaggagggc caagagcagg acaggtttgg ggcttggttc |
| 60721 | tggttagtcg aggcagagcc ccttagaggt gtttggattt gccatggtga tgcaggggac |
| 60781 | ctgagtgtgc tggttgtcat gagatcttgt tgctgggaga acagtcaaga acagggtaga |
| 60841 | aaggcccttc cccagcttta gtgtgccttg gagcaggcag ctttgaatag ggccagcttc |
| 60901 | ttcagagaga gctgccagaa tgtcagagag gcgagaggtg catttgttat tttaacaatt |
| 60961 | ggattgggtc ttcactggga gctctacaaa gaaaccaaat tagtgaagtt agattgccta |
| 61021 | tcccaccaac aatggatgtt tatggtatga gtttagctct gtgctagaga atctcaatat |
| 61081 | atttagattt gggcttgaga gagagaactt cagtgtgatc tgagactgaa cttccattca |
| 61141 | cattcattcc tcgaggaagc atgttgagga tctatgtgta atatactggt gttccattta |
| 61201 | cttgaaatat cctggcttgc tctattcctc tcacttaggc actcactcct ctaaaaaata |
| 61261 | aatgtgtata tgtgtatgtg catgtgtgtg tacatgtagg tgtaagtgtg cataaaatat |
| 61321 | ggaatagact gaaatcaaca aacttataaa aataccaaat aacctcaatc tgtaatttta |
| 61381 | tttttgagaa gcactaaaaa tgtcaataaa tgaattgtct gaaaaactaa tcctttggct |
| 61441 | ggtgagataa gtctgagttg ccagaaccag ttaaagcttt atataatttg gcagtacatt |
| 61501 | tgcgttcacc tgagatttct ctgcctccag ctgtacaaaa attctgtgat atgcagagcc |
| 61561 | tcgaaacctg ggccagaatg ctaaaactaa ttttctttcg taatctgagc cgtaattgca |
| 61621 | ttttaactca gttctagtgt gaagactgct tttgtatgtg tgtgtgttta cttcatgtca |
| 61681 | ttaatttctg gcatttaagt tagtgcctgg cacatagtct accctcagta aatgttagtt |
| 61741 | cttactgccg agtctactgt tctttccttc taaaccagtg gggtagatgc ttcctttgtg |
| 61801 | cctcagtggt tcattccctt gccctcttcc cctctcctca cccttgagtt gttagatcct |
| 61861 | ggttttgccc caggcaggtg ccaaccccga ggaactcctg accctgtggt gatgcatgcc |
| 61921 | tgcatgtgtg cagccagctg ggcagcttgg aaagccatag tattgacttg ccaaataagg |
| 61981 | actagtttac aaaagtcata agagatcact gtcatcaact gctatcacta tctattatcc |
| 62041 | tttctctttc caaaacacaa tttaggtgca taataaaaac atctgtagaa ttactctgtc |
| 62101 | aagcctatgt atgcccatca gttgtgcaag agcctcagtt cctacttcta atgaatttta |
| 62161 | tactgattat cccagacctt tctgccagct ccctaatagc agaggagaga atacactgtt |
| 62221 | ggagtcaggg gcaaccaaag cagctagtct gagtatagaa tttctttgaa ttctcatctt |
| 62281 | taagaaccat gctcagctgg gcacagtggt tcatgactgt aatcctagca cttcgggagg |
| 62341 | ccaaggtggg aggatagtaa tggcctggtg tggtggctca tttccataat cccagcactt |
| 62401 | tgggaggcca aggtaRgcag attgctggag tccaggagtt caagaccagc ctgggcaaca |
| 62461 | cagggagacc ccccatctct acaaaaaaac aaaaaagtag ccaggtgtga tggcacttgc |
| 62521 | ctgtagtccc agctatttgc agagggctga ggcgggagga tcacttgagc ctgggaggtt |
| 62581 | gaggctgcaa tgagctgtga ttgtgccact gctctgtagc ctcgaggaca gcgtgaccct |
| 62641 | gtctaaaaaa aaatggatga tatatccaca tgattcagaa ttgaagaggt acaaaacaat |
| 62701 | aaccaataaa ctctttctcc ccactgtgtc ctagccacct tgttttcttc cctggaggca |
| 62761 | tccagaagcc aattttgtta gtttcaactt ctggagatgg cttatgaaaa tataagtaaa |
| 62821 | tgcatctata ttcaacccca ttttagaaga aatgatacct actatatcag ctgctttctg |
| 62881 | tgcaccttgc tttttttctt tttcacactt tagcatgatg atttttctgt attggcacat |
| 62941 | aaagagcttc ttctttgtta gttggtggtt cttatggcag acaactgaat ccaccctaga |
| 63001 | tggttaagcc aaaaagggat tcggatttta ggtaactcat ggaatccttt ggagggctgg |
| 63061 | aggaacaggc tagcatccgg gtgcccagaa acaaagcccc aagacacacg tagtactagc |
| 63121 | attaagtgga agccacctgt gctgctgcct ccactgaaca gcagatgcta ggtatttgac |
| 63181 | ttgactgcaa ctgcctctac ctccactcct gcaccgggaa ggcaacctag cagtgacacc |
| 63241 | tgcaaaagta gaagctctga gcagagcctc tttccttaca tttctcactt ctaaagcaaa |
| 63301 | atctggtgtg gatgtatctg attagtggtg actaggtgac acatttgcat cttagcttca |
| 63361 | atagagtcta ggaattttat tctcaacttt taaacttgga ggtgggactt gtaataggtg |
| 63421 | aaatttttgc aaatagagaa ataattttca aagtatgatg gacagtcaca gatatggcaa |
| 63481 | atgtccacta gagctgctta gaattccatt gtatggatca accacaattg attaaactaa |
| 63541 | tatcttagat ggtagacctc taagtgtttt ctagttttct agtctgtgac ttaggcaata |
| 63601 | ctgtattaat gaccatatac atgtgtcatt ttgctaatgt aaaatggaat ataaatttct |
| 63661 | agcagtggaa ttgctagatc aaaaggtaaa ttgtaattct tgtggatatt gccaaatggt |
| 63721 | ttctggatgc tatactgatt ttttgcttcc actagtgatg ttcctgattt tttttttttt |
| 63781 | tttttttttt tgcacagagt cttttccttt tttagattcc ttctgagcca gagtggcagg |
| 63841 | ctgagtgttc cctgctactg gggtgagttt tgggccttgg gccaagaatg ctgacaaatc |
| 63901 | agatctgggc cataacctat gccaatatag actgtattta gcatctggac tggatttggc |
| 63961 | ttatagttta ttgtagtcct tctagcccta aattaaatgg catcttgggg tatctaaaag |
| 64021 | ataatgttgc ttccaaaatt gatttgcaat gtgggagtag cttacatctt ggattgttat |
| 64081 | gtgttcacaa atgcataaag atgttttcaa agaatggttc taaagaatta taataccttt |
| 64141 | tctttttaaa aaagcaaaat cttggccggg cRcggtggct cacgcctgta atcccagcac |
| 64201 | tttgggaggc cgaggcgggc ggatcacgag gtcaggagat cgagaccatc ccggctaaaa |
| 64261 | cggtgaaacc ccgtctctac taaaaataca aaaaaattag ccgggcgtag tggcgggcgc |
| 64321 | ctgtagtccc agctacttgg gaggctgagg caggagaatg gcgtgaaccc gggaggcgga |
| 64381 | gcttgcagtg agccgagatc ccgccactgc actccagcct gggcgacaga gcgagactcc |
| 64441 | gtctcaaaaa aaaaaaaaaa aaaaaaagca aaatcttatt tctagtgtgt gaactattgc |
| 64501 | atagattttg ccaatatctg aactatggca tccaattgct ctggttggtt gccatgaaag |
| 64561 | tttatagtaa cataggaagc tcctgtttga aagctcctga aaaatttaaa caaatgtata |
| 64621 | taaatgggtg aaaaatgggg gctgaggaag aaggtgaata catgtgtttt cttctcatgg |
| 64681 | aagtgttcac tattttttat taaatttttt ttagagacag ggtctcacta tgtttctcag |
| 64741 | gctggtctcg gactcctggg cccaagtgat cctcttgtct cagccttcca agtagctggg |
| 64801 | actacaggca cgggcctcca catctggctg tgtttgcagt ctttaagttg gcatttacct |
| 64861 | agtgcagttg attatttcaa gaaaatacac tttctacatg gctactgagg gtatctgaaa |
| 64921 | ggatataaag tatagcccag ggaagtagca tgttgttagg caggggttag taactctttc |
| 64981 | tggaaagggt cagttggtat tttaggcttt gcaggccaag aaacagaatc aaagatatta |
| 65041 | cataggtaat tctataacaa gagaaaaaat aaacttctac aagcttttta ttgacaaaat |
| 65101 | tcaaaacata ataactgagg actttttttt ttggtaatac aggtctactc ataagaagaa |
| 65161 | cagcattctt actttgggga taacattttg cttaatggga ttcaaagaat agtgttccct |
| 65221 | ttcatcaaat cgattgcaaa tgttcatctg taaataccat ttattttctc cttgtgggcc |
| 65281 | atagaaaacc aagcagtggg ctggatttga cccatgagcg gtagttttcc aaccactgat |
| 65341 | gtaatggaaa gaccatgagc tttggacctg gatgcatgta ggtttgagta ctggctctga |
| 65401 | atttgtgacc ttgaacatat cacttaactt tctgaattgg gaaggtttat ttgtgtataa |
| 65461 | actgagtgta atgacaccta tctcatggaa aaaatattac ccccatcttt gtgattttgc |
| 65521 | attagtattc tagttctgta gatgctgata tgatttagct agacttacct agaaaattag |
| 65581 | tatttaactg attttaaagc tgacttttca agggatagaa cactcacatg gtcttaaagt |
| 65641 | acaatgtaaa aacatagctt tgcaatgaag agatctggca gtcaccacat taatcaaatg |
| 65701 | accaagcttt gcaatcagta ccaatggtgg gatgttctga cattgtgtgc ccctcactgc |
| 65761 | tgtgtacctc tcactatatc tcagtatcac ctgtgtggtg ttcttgccag aaacttttga |
| 65821 | ctttaatgta atcacaataa aacagacaaa tccaaaacat gggatgttct gaggacaact |
| 65881 | ggcctggatt cttaaatgat tcagtgtcat taagaaccaa gggggattgt tctagattag |
| 65941 | ggggagacta agggaaaatg agataaaaat gcagtgcatg aacattgact aaatcctgga |
| 66001 | tgaggaaaag aaaattcagc tctgtaggca ttttctggat aatcaaggga atgtgaatgt |
| 66061 | ggaccatatg ttgtatgtta gtccattttc acgctgctga taaaggcata tccgagactg |
| 66121 | ggaagaaaaa gaggtttaat tggacttaca gttccacatg cctggggaag cctcagaatc |
| 66181 | atggcaggag gcaaaaggca cttcttacat ggtggtggca ggagaaaaat gaggaagaag |
| 66241 | caaaggcaga aacgcctgat aaacccacca gatctcatga gacttgttca ccatcacaag |
| 66301 | aatagcatgg gaaagaccga tccccgtgat tcaattacct ccccctaatt gttcaagtcc |
| 66361 | ctcccacaca cgtgggaatt ctgggagata caattcaagt tgagatttgg gtgaggacac |
| 66421 | agccaaacca tatcatgttg tatgataata ttgtgttaaa gttgatttcc ttgggtgtgg |
| 66481 | tgatgatggt gcggtggtgt acgaaaatat ccctgttctt agaaggtgaa tagtaaagta |
| 66541 | cttaggggtg gtgtgtcatg aggtgtgtct gtaacccact tttcagatgg tttggcaaaa |
| 66601 | tgggagggaa ggagggaggg agagagagaa tgtgtgtgag tttgtgtgta tgtagaatag |
| 66661 | gtgattctgt gcattgctgc tcagttctta gcatagcact cagcacattg cttttgatag |
| 66721 | ggaggtgccc aaccaaaact tgttaaacta acaagtactt taaggtttta gtaggattct |
| 66781 | ctgaaatcac acctcagcac tgcctgtctg ccctctcttt agcagtgtca tttggcttag |
| 66841 | gcatatacta taatgcctaa tgttgggtaa tcagatatgg gggttcatgt tttcttgagt |
| 66901 | agctcaccca tgtccctctg gactgaaagt ctctcttgaa tgttacagta taaatgaatg |
| 66961 | ggaaaggaaa acccagcata gaggttgacc tgcgtgttca gctgttccct gagaggctga |
| 67021 | ggtacaattt gtaagcatgt acttcagatt ctcttctgta tttctgttgc tgagtttgcc |
| 67081 | cacttgggga gggttgacac ctcctggggc tgtgctgagt aggaggtgca tctctggagt |
| 67141 | tctgactggc tggagcatca ctgaggtatt ctcctgggct atgagggcta gggccaggat |
| 67201 | aaagaatggt ggatgccttt ccttatgcct tgatcctaac cggtaccctc ttagctgagc |
| 67261 | aggaagggaa cagagaaatt aatattctct tatattcctg caggaactct tatggctata |
| 67321 | tggtgtgaga tgactaaagc caatttagca gctacttgta aaagtaataa aaatggaggg |
| 67381 | gaagaagaaa gattctgatg caacaggctg tgaagactaa ttcaacattt atttattgag |
| 67441 | ctactgagca tatagcaggc atagtaaagt atgagtcctg ggggatacaa ataataagaa |
| 67501 | tataccctcg ctttctgctg ggcacttagg gcatagggaa aggagatggg ctacagtgga |
| 67561 | gataattatc aaaataccaa ttgtgctgtg ttgcaaggct agggatgtgt tagaaagcta |
| 67621 | gtaaaaagtg ttatgggaat gcagagaaca tgccatgtgg aattgactac agtttcaaag |
| 67681 | ataaggttgg tcttgaggtg acttgaagaa tgagcctggc aggcaaagaa atgggcatct |
| 67741 | ggtgtgacta aagtcttaat tagctgtagg gtggggagga gagagacatg acgatgttgg |
| 67801 | aaaggtagtt tgggccagac aggagggcct cagaagccat gcttggtttc acaggaaggg |
| 67861 | cgtgaagagt cccagaagcc ctttgaagtt ggagggtgat gtggttttga taagaggtca |
| 67921 | cggagggaat gtagaagctg gtgtgtgaag gtgtgtgagt ctggagacag gacttgaaac |
| 67981 | agtcgaggcg agagcaaaga atctggattt tgctagctgc agcagaggga gagtagaaaa |
| 68041 | tggggtgctc ttggagaatt cttccagccc ctcctactag gagtttttac attttcaaat |
| 68101 | ctttctaagg aaacctttaa taatacttga gagaagctta tttaaacctt tgaacagttc |
| 68161 | tgaaaatgta ttacatttca gccaggaaag aaagtattat ttcaggcaaa ctggaacatg |
| 68221 | gttactcagc acagtcgcct aatcttatag aattaggtag gagggaagct atgttagtgg |
| 68281 | ccttcaacct tcttccagaa ctttgttcag atttctccca aatggtcggt gaatttttct |
| 68341 | gatttgtatc cagcatccca gccatttagt gtacatccgt aagctgtaag gaagttgatt |
| 68401 | taatctattt ctttttttca tctcttgcct tccacctgag aaaacactac tccataggtt |
| 68461 | cttattctgt attaggaatt tgcacaatgt gtaacctaag atggatccag ctgggagttg |
| 68521 | gatccagctg ggagtgttac aggtttggag tttagactaa agaagaggag tggaatcttt |
| 68581 | tcattttttt cccctttatt ttgaggaaag cccagtgatt tggattgttt gaaggatcaa |
| 68641 | ttatatcaaa tatctccact aattgagtat ctgggataaa cttttttttt caccttattt |
| 68701 | tctttagata ccattaagca tttcgaccca caacttacag cttttccatg gagattttaa |
| 68761 | acttagaaaa ctttgaacct tgatgaatga aaaaaacctt tcagaactaa cctttttttt |
| 68821 | tttttttttg attcagaagt ccatgtgtaa gttccaaagg cttataattc ttttctcttc |
| 68881 | cttgaggctt gactatttca gaacgttctt ggtatgtctc ttggaggttt ggtgtacaaa |
| 68941 | gagcaaggaa agtaacctat gggcttaact cacttatgtg tatatattct actttggctc |
| 69001 | caaagcaatg attctagtgg tgatatggga ccaagggaca gatttcaagg taaataaggc |
| 69061 | tgtttactaa tgtcagatta aggtattagt cttcaagtac cataaatgtt ttactagcaa |
| 69121 | tttcagttac ctgtagtaaa gagctttcca gttccatgtg gtagttatgg taaaggagca |
| 69181 | actgaaagtt aacttgatag tagattaaca ctcactgaaa atatagttgt tattttttca |
| 69241 | cataccaagg attacaatgt cttttttttt Ntctattttt aaaatagaga cagggtcttg |
| 69301 | ctatgttgcc catagtcttg aactcttggc ctcaagcgat cttccttgag gcctcccaaa |
| 69361 | gtgctgaggt tataggcatg agccattgtg cccggcctac aatttcttaa tattaggagt |
| 69421 | tattttgcaa gacaactatc tcaatgttgg aactacacta atgttggtgt gtttttttcc |
| 69481 | ttgtgtggga tagtctgtat cttaagatgt gtaatgtata tattcacaat aagggctaca |
| 69541 | ttataaaatt cagagacatt ttatttttga ggtaggtttc attcacttaa ttgagaattt |
| 69601 | gttgactgta tcttgggtgc ctatccttat getaggagat gaatggaagt tgccccctgc |
| 69661 | cattgaagag aagacatttt aatgggaggc agagataaac agataattaa ttataatgtg |
| 69721 | atacatttgg aaatagtcac aaaaaacttg aatatggaga ttttcaaaag tacaaagaat |
| 69781 | aggatattga accctcatga acccactacc aaacttcaac aattactaac tcttggttat |
| 69841 | gcttgtttca cccatgtgcc ccacctcctc ccttccccct tttattttga agcaagtcta |
| 69901 | agacactgta tcatgtgatg gacattaata gatacttgga tagagtactc tgagaataca |
| 69961 | ctgggaaaac atactgtatc gggacaggga aaggagctac agaaaagtaa catttgagtt |
| 70021 | caatctaggt ggggaggagg atgttgttcc aggtgatggg atggaaactg acaattatta |
| 70081 | caatgtcttg aaaaatgtgt atatacaact tttttgttcc tctgaactga gcacccaaga |
| 70141 | tgtaatttcc aagattagga aattgttttg cctgaggact ttataaaaat aattaattca |
| 70201 | cttttaattt agataaacta gaataatcca tgagtagtgg taaYgactaa tctgttctct |
| 70261 | tttcacccca gtgactttct agactttgaa cattagaatc cactccctga aaaagtcact |
| 70321 | ctcctttgta actagagcaa cagctatcct ttgtcacctt tatttttctc tttggaagag |
| 70381 | gaggaataat cagtggctca cttctgaaac atcatgaata ggtagaattt agtaatctga |
| 70441 | attaaaaacc tccctgtaaa ctgaaggaag aggaaattga ccagtgggca cacagaggtt |
| 70501 | ctcaagggac ttttttggtt agccactagg tgaaatcctt gatatttcat catgtacctt |
| 70561 | gtctcatttt cctgttgtga aattgggtgt tcagcattgt ttttcttgtc tgttctcaga |
| 70621 | tcacagaact gtgtggtgca aagcgggttg gttattttgg tccaacacag ttttacattg |
| 70681 | ccctgaaatt aattgctgca gcacaatctg gcctcccggt acggatagag agtattaaat |
| 70741 | gtggtgagta tctcacattt gtatgtttta ttgtccatgg cgagtcgctc atttatgaga |
| 70801 | accattttta atgagtttaa acttttgtgg aatttctcct tgcttcctcc aactcttctg |
| 70861 | caaaataaaa taagaaaact tggcaggtag gtagcttagt ttctttcttt tttaaatggg |
| 70921 | gaaaattctg agagtaacct gagattagtt cattttaact gatttgcagt taaaatagat |
| 70981 | tttattcttt agcaggaaga gattgggcca ggaattctag agcctgtggc aaataatcac |
| 71041 | attgacttta attttccact tgaggtatct ataatgttat tgaaggctct gttagaggag |
| 71101 | taacaaggga gtttgagtgg aactatgcct tttctctttc ccatacctgc cagtgctcag |
| 71161 | cctttaattc ttagtttcca aagcactgtt ctgatgctag taagtacttg ggtgctacca |
| 71221 | aaattttggc agaatgaatt tatcttctta aggttctgat ttaatatttt ctcatcttgc |
| 71281 | catccctgcc ctcaacccac tgtatgcttg agcactttct acaatttaaa caaaaagcaa |
| 71341 | aacccccaac tattagcagt atttctgctg cctcaggatt ggttttaaaa atctgtatga |
| 71401 | aaaaggtgat tattcaaggg gtgtgccaag gtcctctgat aattcagttt gatacagatt |
| 71461 | actttcatat gtccatacag gtatgtagga actttttttc tttttctcat tctgagacta |
| 71521 | aaaacctctt aactgctcat gcctgtcctg tgaagtctct gatagacata caagtggatt |
| 71581 | caggatttat ggtgagccca tggaaacctc gtaggacaat ccaaatggcc gagaagattg |
| 71641 | ccatagatgg ctggctgtgg aagaccaagc ccttcacact aaccgtYagc aggtggcagt |
| 71701 | catcatgcta atctttatca gggggctagc ttgtgccaga cactttcatt taatcctcat |
| 71761 | gacaccctac taaattaggt acttgtgtca gtttgattgc ttaggataag ctgcgataac |
| 71821 | aaatgtcaca atctcagtgc ctcaaccaga cagaggttcg ttttccactt tgcagtgggt |
| 71881 | cagggtgacc ctccagggca gctgccctct atgtggtgac catgtgatcc tggctgcttt |
| 71941 | atcttgtggc acctcccaac acaacacctg ttcccataat cgccacacca gggaaagagt |
| 72001 | atctcaccct agcggttaaa tgctttggcc cagacatgac tgaagttatt gctgcccaca |
| 72061 | ctgcattggc cagaacttgt cacattaatc tgcttcagtg cagagcaggt gggagaacac |
| 72121 | aattctctcc tgtgcctgca aggagaggac aactgcgagt ctctactatg gctgcacact |
| 72181 | gcaagtctct actatggtta tggctgcaca ctgcaggtct ctactatggt gctgccgttg |
| 72241 | ttcgtatctt acaagtatgg aaatttgaac tttagggagg ttgagtaact tacctaagaa |
| 72301 | tataaggtta gtagaatttg gagccaggat ctacttccag tccccatgct cttaatttct |
| 72361 | atgcaaaata gccttgtctc aattttttct atttagtaag atgctgacag taatctgtta |
| 72421 | tacaatattt tctgaatggg aaaaatattg gacttgaaag gcatctcttg gtatgacaga |
| 72481 | attttgttct ttgaaatagg tttcttgatg gagtgtcatt tgacaggcag gcttagtatt |
| 72541 | cattgctgca agacacagcg gccatttgct ttgattagtt gaaagttgca tcacactgaa |
| 72601 | tttggatttg tggtaggcac tgatagtttt tattccattg taaatgatca ttaagcttta |
| 72661 | ttatagtatc ttccaatttt atcttctgga aaagttagag ctgctttatt gtatacagaa |
| 72721 | taaacttttg acacttttgt tacttatttt aagttagacc taagtctttt acctgatttg |
| 72781 | tgcccagatt gacagtttaa tttcctgagg gcagggagca ggattgttga agttatcact |
| 72841 | tgtgtaaagg attaacagtt tatcacaaat ataaggcttt ctcagtttta ttttataaac |
| 72901 | ccagttgctg ccttttacct ctgagagttt atatatgtcc agagttaatt ctgaattatc |
| 72961 | ttccttcacc actctctact aatccattta aagattgatt aaataaaatt atttgcagtt |
| 73021 | agcatatgtt gaataagaat tagggcaaga ttgggccaaa ctttgagctg ccacttagaa |
| 73081 | ttttccagtt tgttgcttga aatctattag cttaatgctt gatgacacaa atcttgaagg |
| 73141 | ccaaagggat cactctgtga gaacctaatc tgaaaggctg tgctagccct gtcacaccca |
| 73201 | gggcatcata cccagggagt tgtggatgga tacccttatg tcatgaataa catgcacctg |
| 73261 | tgtgtatgtg cgtgaccctc taatccctag tccttcccat ttcggtaaat gccaccacca |
| 73321 | tcctcagctg cacaagccag aaactcagca gccatcctgg attcctcctt ttccctcact |
| 73381 | ccttaataga atccatcaaa agtcctagca atgtggctcc aatctcactc cctgttgcct |
| 73441 | ctatctcttt gtccctgcta gaccagacaa ccttcatcta cttacctgga caccacaatg |
| 73501 | gattgacatc ctagcccctc ctctcttctc tactgtgatc tctcttttcc cactggtttt |
| 73561 | ttgtttgttt Nttttttgac agagactcgc tctgtcaccc aggctggagt gcagtggcac |
| 73621 | gatcacagct cactgcagcc ttgacttcct gggtgcaagg gatcctccaa cctcagcgcc |
| 73681 | ccgagtagct gggactacag gcatgcgcca ccactcccta ctaatcctac taatttttgt |
| 73741 | attttttgta gagatggggt tttgccatgt tgcccaggct ggtcttgaac tactgagctc |
| 73801 | aagtgattgg cctgcctcag cagcctccca actttttttt tttgagacag ggcctcactc |
| 73861 | tgtcacccag gctggagtgc agtggtgtga tcacggcttg ttacagcctc tgcttcccag |
| 73921 | gctcaaatga tcttcctgcg tagctgagac tacaggcata catcaccatg cctggctagt |
| 73981 | atatatgtct ttttgtagag atgggggtct tgttatgttt cccaggctgg tctcaaactg |
| 74041 | ctgagctcaa gtgactctcc tgtgtcagcc tcccaaagtg ctgggattat aggaatgagc |
| 74101 | caccataccc agcctctttt cctacttttg aaagtgtgat cttttcaaaa tacacagcag |
| 74161 | attattccat cctcatcctt taaaccctcc agtagtctgc ttgttgtact ttgtaaaaca |
| 74221 | ttcaaagtcc ctacactgat ctgtaacacc ctacacaatc tagttcttgc ctacttttcc |
| 74281 | atcctcatct caaatcttaa acccttgctc actacctgtt ccccagtctc agtggccttc |
| 74341 | tagccatttc tgggtgactt ttcctgctgc aggccatatg tccattttat cttgcagtct |
| 74401 | tgggttccta gggatctgtt tttcctttct gcttcccctg gttggctcct tcccaacctt |
| 74461 | caggattcag agtctgtccc tactatccta cctaaagtag ttctgtcaga ttcattaatt |
| 74521 | catgaaggaa ccagcatatg gtaaaactgg ctcaagaaag tatagagagt cagatttatt |
| 74581 | tatagtcata gaaagaaagg atgctggaat agaattgcta aattaggtat aaagctaatt |
| 74641 | aacgccctac ggggtaatac aagtgcagcc tggctattgt acttttttct aagatgaaag |
| 74701 | tatttgcatt gccgtcagac aagaattact tgttagctaa cagttttttc caagttaata |
| 74761 | tctacccttt caggattatc aaatgcccaa ccaagagtaa acagtaactc agagcagaag |
| 74821 | tccatatccc taggaagtga ggtgatcctt gggtgggcct gctgggtaat gcttctgcag |
| 74881 | ggcaatctcc tccccacttt gttctttccc agcatctgtt tcctgatagc acttagcaga |
| 74941 | gtttgcaaat attccttaca ggtttcacta ttttttttgt ctgtcttcct cacttgtcca |
| 75001 | gtttgtgtaa gggggaggga agaggatctg acaccccttg cattgtgggg cattataggc |
| 75061 | actctgtgtg tattggttga atgaatgaat gaagaggtgc atgtctacgc atgaggaggg |
| 75121 | tgtcttaatc atctgccact ctttaatcta aaacgattgg aattcagatt ctcctggaac |
| 75181 | tccagaggag tttttacaga agtcatctca gtctgcatat catctttctt aggcactgat |
| 75241 | tttcaacctt gtcggcactt gagaaccacc tggagagctt taaatataaa ataaaaaaat |
| 75301 | acagatgcct gggtccatct ccagaaattc taatttcata tatctgcagt ccggtctgag |
| 75361 | catcagaatt tttaacagct ctccaggtga tttttgtatg gagacatgtt taagaataaa |
| 75421 | agctgcatca agcattttta agagctttgc ttaaactgtg tgtgtgtgtg tgtgtgtgtg |
| 75481 | tgtgtgtgtg tgtgtgtgta gttacagtag ctgcatctga actagggtgc tgtacgacac |
| 75541 | agggcacatg atttaagggg gcacttactc tcagggcaag tgccgactct acatttacac |
| 75601 | caccctgaga gtgcgtgcct ccttaaatgt tggcactagg tatctctcta gccccaccct |
| 75661 | cgtctgagtt ggggttaaac agatggcaaa acagctaggg cagaaggaaa gaaggtgaaa |
| 75721 | attaaggact ggtagagaat ttctgatgag gaaatcatgt ggtgaacatt tgccctggga |
| 75781 | cctgctgtta cctttctttc cagggtctga agttgagcac gtgtgaggac tgagggaagg |
| 75841 | ttaggcagaa cctagagtag ggtttctcat catttttgtg ccaggagccc ctttggcagt |
| 75901 | ctgaagcctg tgggcccttt ctcagaatta tgttttaaat gcaacaaaga cataagatta |
| 75961 | taatggaaac caattatact gaaataatca ctaaaatagt taaattgtta tataacaaca |
| 76021 | tacatgcttc taggcctggc acagtttctc atgcctgtaa tcccagcact ttgggaggcc |
| 76081 | gaggtgggcg gatcacttga ggccaggagt tcgggaccag cccggcaaat gtggtgaaac |
| 76141 | cttgtctcta ctaagttcga gaccagcctg gccagcatgg tgaaaccttg tctctactaa |
| 76201 | aaatacaaaa attagccggg tgtagtggca catgcctgta atccctgcta ctcaggaggc |
| 76261 | tgaggcagga gactagctta aacccaggag gtggaggttg cagtgagtcg agattgtgcc |
| 76321 | actgcactcc agcctgggca acagagtgag actatatctc aaaaaaaaaa aaaaaaacca |
| 76381 | acccccaaaa catatatgct tctttattaa tgcattaaat aaagcatcta acagtgtttg |
| 76441 | aaataaccac tactttttca aattagtaat atgcataaac agtacttcaa gatgttggcc |
| 76501 | cggcatggtg gctcatgcct gtaatcctag cactttggga ggccgaggag ggtggatcac |
| 76561 | gaggtcaaga gattgagacc atcctggcca acatggtgaa accccgtctc tactaaaata |
| 76621 | gaaaaaaatt agctgggcgt ggtgacacac acctgtagtc ccagctactc aggaggctga |
| 76681 | ggcaggagaa tcacttgaac ccgggaggcg gaggttgcag tgagccaaga tcacgccatt |
| 76741 | gcactctagc ctgggcgaca gagcgagatg ccatctcaaa acaacaacaa caacaacaac |
| 76801 | aacaacaaca acaaacaaaa ccctaatgtg ataggtgaca aagttaccgg tattgctagt |
| 76861 | gataatacag tggcttgttg cctacattca taatggaaag aaatgctaaa tttcagttat |
| 76921 | agtagggaaa atgaagttgt aatttttttt tttttcctaa tcaaggtgac agaccccgtg |
| 76981 | atttcttttc actagtccct ttgggagcct gtgaactctg atggtgcaat gacatgattc |
| 77041 | atgtagtctt tggggctgtg ttccccaaac tggtttatgg tctggttgca gtctggcaca |
| 77101 | gtttagaatt aggacaaaag gatgagttat ttataaagct aaatgtattc aaatctaaga |
| 77161 | ttgggttttt tttggttcct cttgttaagt gtcctttgtg aaactgctaa tgggtaatag |
| 77221 | tttggggtaa aattcttgac gagataaaac attggcaact ttatgttggc tttcttcatt |
| 77281 | tctttttttg ttgttgttgg gggagagatt tttcctattt tgtgaaatcc ccaaatttgt |
| 77341 | gagccactgt tgtggtcacg attctactgc caagaagata ctgaggcaaa tcattgaatt |
| 77401 | ccctttgact cctcccttat ctgcaaaaca gttgttctcc acctaggtga tttcgtccct |
| 77461 | ttagggactt tcggcaaggt gaggagacat ttttggttgt cccagctgcg gaaggggact |
| 77521 | gcctggcatc tagccggtag aggccaggga tgctgctcaa caccctacac tgcagaggac |
| 77581 | aggaccccct cttccaacaa caacaaactt atctaatcta aaatgccagt aatgttgaga |
| 77641 | ctgagaaagc cttctgtaaa ataaggatca agacttagga actctaatcc tgctgccttg |
| 77701 | cttcctgtgc tctagttagt ttttttgttt ttttaatcat acaagaaagg tagaaggatt |
| 77761 | ttgcagcatt gagtcctatt tctgtggctt ttgttttcaa agaagaatta ttttattcaa |
| 77821 | ggaaaaataa aagcagttat tctctggttg aattgagggt ggggttccct gtgctttgcc |
| 77881 | tatttgacaa accctcccta tctcttaggg ctaggagaga aatcttttga gtgtgacaac |
| 77941 | cagtttggta attcatagaa aagacaggat aggaagccca agggtaaaga aagaagccca |
| 78001 | ggaggtaagg ttgtagctgg ctctgtgtgt gtgtgtgtgt gtgtgtgtgt gtgtgtgtgt |
| 78061 | gtgtccacac agtgtctctg tattttgaac actaaatact atgttgggat gtctcattaa |
| 78121 | gctaaggtca actcatcctt tctgactgaY gtccaaccca atcccccttt cctttttttt |
| 78181 | cctcagtgtt cctttgtcaa tttcctttac cccctctccc agccctgcta cagggacaca |
| 78241 | gagctgcttt ggacaaagtt cgcctatgca ggttcactga ctgtacggct ggacatttgt |
| 78301 | gttttctagg ggtctgtccc tctaccccat agcgctgtca ttagctctat tgttgctcct |
| 78361 | ccccaaaggt tctccccaac cttttgagct gtggattttc accttatttg taaaaatctt |
| 78421 | tgttgtggga ttctttgaca atctagtaaa agctctggat cttagcagga aaatacacat |
| 78481 | tgacattatg taaaaaatgt tgccttcaat gtcaggggtt tctggacccc tagagctggt |
| 78541 | tcatggagcc cagtgaagag cccttcttcc agagaaatgc agtgtatggg atggatatag |
| 78601 | aggaggtaag ttggaagatg catagtgggg aaattgaagg ccaaagaggt aaaataaaag |
| 78661 | ggatgctgct tgctagcagc agagcccagt ggattgtgga cctcctaact cactcctggg |
| 78721 | gttctacccg tggtatggcc aggaaattga acttatccac catcctcctc tgccaaacat |
| 78781 | ttacaagatc acccttccca ttgtaacctc caccccgcca tctagcgatt acctacccat |
| 78841 | ttcctaattg acaagaggta tttcagatca atgggcagat aggaggataa taggagacta |
| 78901 | gatagctttg attactcagg cctgggttga taagactggc ctgatttttt tttttgtttt |
| 78961 | gttttttgtt ttcccccaaa gtaacttcag gaaaatgtgt agggatggtt tcactttgga |
| 79021 | attatagaac attattgtca taattctttg ttaacgtata agtagctggt ataagtgaat |
| 79081 | aggtaacaaa aagcagtggg agagaaggga gccagcagtc aggagtcaag aatttttgtt |
| 79141 | acggtcttag ctctgccact gtattgctgc atgactaagg caagtcttgc tctctctttc |
| 79201 | ctgggcttat ttcctcacct gcaaaatgca gacatttgag caggttttct ctagattatt |
| 79261 | ttctgaaatg ccacagtttc atgctaaatg ttcaaatctg tactagaaag aattttaatg |
| 79321 | ttcaggatgg tagtatagcc ttgtggctgg gaatgtaggt cctagcaata ggctacctca |
| 79381 | ttatgaatct ttccttcatt accttttatt tgtgtggcct ccggccagtt tcttaatttc |
| 79441 | tctgtttcaa atgactccct ggaggaaaag gtataccaat aacacctgcc ttatagactt |
| 79501 | gttgtaagca caaaatcagg gaatccctta tcaaacattg tagaaaagca tctacaactt |
| 79561 | tgttatttaa tatcgtagcc acttgccatg tgtggctatt gagcacttga agtgtggcag |
| 79621 | tgtggctagt gtgactgaga aactgaattt ttcattaaat gatttaaata aaaggtttta |
| 79681 | aacagtgtaa aatattttct gttaaatgca accttattga tttagtagga ccacttttca |
| 79741 | cttcaaccat caaattctgt aagacaaggg tcaacaacct ttttcttttt ttttcttctt |
| 79801 | ttttaaattt tattttatta ttattatact ttaagtttta gggtacatgt gcacaacgtg |
| 79861 | caggtttgtt acatatgtat acatgtgcca tgttggtgtg ctgcacccat taactcgtca |
| 79921 | tttaacatta ggtatttctc ctaacactat ccctcccccc ttcccccacc ccacaacagt |
| 79981 | ccccggtgtg tgatgttccc cttcctgtgt ccatgtgttc tcattgttca Rttcccacct |
| 80041 | atgagtgaga acatgcggtg tttggttttt tgtccttgca atagtttgcg gagaatgatg |
| 80101 | gtttccagct tcatccatgt ccctacaaag gacatgaact catcattttt tatggctgca |
| 80161 | tagtattcca tggtgtatat gtgccacatt ttcttaatcc agtctatcat tgtgggacat |
| 80221 | ttgggttggt tccaagtctt tgctattgtg aatagtgccg caataaacac acatgtgcat |
| 80281 | gtgtctttat agcagcatga tttataatcc tttgggtata tacccagtaa tgggatggct |
| 80341 | gggtcaaatg gtatttctag ttctagatcc ctgaggaatc gccacactga cttccacaat |
| 80401 | gattgaacta gtttacagtc ccaccaacag tgtaaaagtg ttcctgtttc tccacatcct |
| 80461 | ctccagcatc tgttgtttcc tgactttttg atgatcgcca ttctaactgg tgtgagatgg |
| 80521 | tgtctctctg tggttttgat ttgcatttct ctgatggcca gtgatgatga gcattttttc |
| 80581 | atgtgttttt tgactgcgta aatgtcttct tttgagaagt gtctgttcat atccttcgcc |
| 80641 | cactttaaag ggtcagatag actttgtggg cagtatgatt ttggtcacaa ctactcaacc |
| 80701 | ctgttgtagt atgaaagtag ccatggacag catgtaatca gaggatcatg gctgtgtgct |
| 80761 | aataaaactt tatttatgaa cactgaaatt tgaattttta atttttttta ttttttattt |
| 80821 | ttgagacaga gtctcactct tgtcacccag gctggagtgc tgcagaggca cgatctcagc |
| 80881 | tcactgccac ctccaccttc tgtgttcaag ctattctcct gcttcagcct cctgagtagc |
| 80941 | tgggactaca ggcctgtgcc accacgcctg gctaattttt gtattttagt agagatgggg |
| 81001 | tttcaccatg ttggtcaggc tagtctcaaa ctcctgacct cgaatgatcc acccatcttg |
| 81061 | gtctcccaaa gtgctgggat tacaggtgtg agccactatg cctggcctga catttgaatt |
| 81121 | taatataatt ttcacatcat gaaatatttt tcttttttct tttttttttt tttttttttt |
| 81181 | gcaaatattc aaacattgtg cttgcgggat gtacagaaat aggcagcaga ctggatttgg |
| 81241 | ttcttaggta tacacccaag ataaataaat tcatgtccat caaaaaacat ttataagaat |
| 81301 | ttcacagcag ctttatttac aatgggccca cactggaaac aacccaaatg tttatcacag |
| 81361 | gataatggat taaaattttc aatatattct tttagggtct tgctctgtca cctagactgg |
| 81421 | agtgtaatgg tgctgtttca gttcactgta acctccacct tccaagctca agccatcctc |
| 81481 | ccacctcagc ctcctgagta gctgggacta taggtgtaca ccaccatgcc tggctaattt |
| 81541 | ttgtattttt tgtagagatg gggttttgcc atgttgccca cactggtctc gaacttctga |
| 81601 | gctcaagaaa tctacctacc tcggcctccc aaagtgctgg gatcacaggt gtgagctact |
| 81661 | gctcctggct caatatattc ttaaaagtgg attgctccat agcaatgaaa aagaatgaac |
| 81721 | tatgtgtaca atacattggg tgattatcac agacaaaaag gtcgtaaaaa aagcttgtca |
| 81781 | caagaacaaa tactgtatga ttcaatgtat gaagttccat aatggcaaag ataatctgca |
| 81841 | agtatagaaa ttagtacaga ggttgctcgg tggaacaaag ggaggggatt actggaaatg |
| 81901 | aacatgaagg aaaattctgt atcttaattt gggaatttgg gtgttggtta cagggatata |
| 81961 | tatatatata tatgtatttg tcaaaattct tcaagtggaa cacttacaat tccctataaa |
| 82021 | tcatcccacc atacagaata ctctttacat aatttaaaaa gaaatcaatt agttgcttac |
| 82081 | atttaacatt gtaataataa taaaatacag taaaatacag atttctggcc acaatggctc |
| 82141 | acattcccat gtagcaacta tcaactagag ctgtgctgtt tcagtatggt agccagtagc |
| 82201 | tgcatgtggc tgttgagcac ataaaatgtg acaagcacaa attgaggtat gcaaggaggg |
| 82261 | taaaatatac atgggagttt gaaggcttaa tatgaaaaaa aatgtaaaat atttcattaa |
| 82321 | tagtttttaa tgttgattac atgttgaaat gataatattg ggttaaatta gattattaaa |
| 82381 | attttgccat taaaaatgtt ttgaaatgga aaatttttaa ttgtgataaa atacaaataa |
| 82441 | catttcccat cttaaccatt ttaaatgtat aattcagtaa tgtgtaaata tagtcacatt |
| 82501 | gttgggtaac tagtttctag aaccttttca cctcacctta caaaatggaa actctgtatt |
| 82561 | cattaaacac cttcccattt tctttttcct acccagatcc tggcaactgc cttttttttt |
| 82621 | tttttttttt ttttgagtgc agtgacgtga tcatggctta ctgctacctc tgcctcccag |
| 82681 | gctcaagaga ttcttgtgcc tcagcctccc gagtagctgg gattacaggc gcatgcacca |
| 82741 | ctacacccag ctaatttttg tatttttagt agagatgggg ttttgtcatg ttggccaggc |
| 82801 | tagtcttgaa ctcctggcct caagtgatct gcccacctca gcctcccaaa gtgctgggat |
| 82861 | tacaagtgtg agccactgca cctggcctgc ccatctactt tctggttYta tgagtttggc |
| 82921 | tactcttgat gcctcatata agtgaaacca cacagtacct gtctttttgt gaccagcttg |
| 82981 | tttcacttct ttctttcttt cttttttttt ttttttaatg tggctcacat tcttttttgg |
| 83041 | cagataacat tgaaagactg tgaacagctg ctgccatttt gatggaacat gagttctctg |
| 83101 | cccagcaccc tccactccat attgtctagc taatgaggct caatgtcact tgccatttat |
| 83161 | tattgcattt atgctgttta cattttttct aatggattga ggaaattgag gtattttttt |
| 83221 | ttcttttttg agtgacaggg tttcactgtc acccaggctg gagtgcagtg gcatgatgca |
| 83281 | gccttgaact ctcagactca agcgatcttc ctgcctcagc ctcctgagta gctgggacta |
| 83341 | caggcatatg ccaccatgcc cagctgattt tttattttta tttttttaag agaaggggtc |
| 83401 | ttactgtgtt gtccaggctg gtctcaaact gctgggctca agtgatcctc ccacctcggc |
| 83461 | ctcccaaagt gctgggatta caggcatgag ccaccactcc cagcctaaag tattcctaat |
| 83521 | atatctgtat ctctatcaaa tatggaaaaa caaaggattt ttctctcttg tagagggtca |
| 83581 | agtgtttaaa aaggagcagg cataattttc tttgtggaag ggaaaaatag tcatttatgt |
| 83641 | ttaatataca aagcatttct gtgttcaaag aatgtaacct aagatgaaac aacaatgaag |
| 83701 | caaattgtca tcttggatgg ccactctcat tgtctcctca gaccctctaa acatttagtg |
| 83761 | tgaactctta ctttgtttgc tgtgtgtaat tcaattcagt ttgggagaca agattctaac |
| 83821 | caatgactac aaatagacct tcttcttgac agacatcatt gtatagcagg tactttattg |
| 83881 | attaattttt tcaaagtttc actattaaat tttgtgtatg tgtgactagg aaaaacattc |
| 83941 | agttgagtat ggtgactcat gcctgtaatc ccagcacttt aggaggccaa gacgggagga |
| 84001 | tcgcttgagc tcaggaattt gagacctgtc tgggcaatat agcaagaccc catctttacg |
| 84061 | aaaaataaaa aaagaaatta gccagcatgg tggcaagcac ctggagtccc agctacttgg |
| 84121 | gaggctgagg caggaggatc agttgagtct gggagttcaa ggctgcaatg agctatgtta |
| 84181 | gtgccactgc acttcagtct gagagacaga ccttgtctca aaaaaacaaa aaccaaaaaa |
| 84241 | aacattcaga ctgaaaattt acaaactact tttacctttc actttttaaa agcttgcatc |
| 84301 | agtttaaggg aaatggcacc ttcatgaaag ttttccatga taagaatatt attatgtatg |
| 84361 | gttatacttc tgttaagtga gagttgcttg gaaccacctg accaaagaaa gtatgagaga |
| 84421 | acttcagctg tattttgtct tttgctgttt ggtggctgtc tgggggagag aggtggtttt |
| 84481 | ctaggcacat agctctaaag aaatgccata gacagggtgg aagaatgtgt gaagtgacaa |
| 84541 | tgtcctagac aatatctggg agccctactt aagtcctaat ataataattg atattcataa |
| 84601 | ttcagttagt gtattccatt gttccttgga ggtatctgtt gaatgccagt aaggcactgg |
| 84661 | tcagggagct tataaatact gagcatttat ttcttttctc cctcaagaag tacttggaag |
| 84721 | tagagtcatc ctcttatggg tagatcaggg tgcaaattat tttatgtgat cttgtttcat |
| 84781 | ctttttagat cctttttcct gactgagctg gagcacatac ctgagaccaa aaccatagag |
| 84841 | ttagcttata tttgatttac taagaatcta gagtttcttt tctcttgctt ttttgctttt |
| 84901 | gaagcccatt cctgctttga gtgctggaga gctgcccgag attgggaaac attaagattg |
| 84961 | gttactctat ttatgtctct tgggcccttg gctgattgat cacacaggga tttgtcatat |
| 85021 | tgcctgcttc tttttttttt ttctggtaca gaattttgct cttgttgccc aggctggagt |
| 85081 | gcaatggcat gatcttggct cactgcaacc tccgcctcct gggtttaagc aattctcctg |
| 85141 | cctcagcctc ctgagtagct gggattacag gcgcctgacg ccacgcctgg ctaatttttg |
| 85201 | gtatttttgt gtagagaYgg ggttttacca tgttggccag gttagtttcg aactcctgac |
| 85261 | ctcaggagRt ccacccgcct ccgcctccca aagtgctgga attataggcg taagtgagcc |
| 85321 | actgcgcccg gccgcctgct gctttgaatc aaatgtgaac atagcaacta aaagtaaaca |
| 85381 | tcatgtggat gttagagaac caggggcctt ctttgcctgg ggaactctag gatacttgct |
| 85441 | tgctgttgct aagatgtttt ctggccttag cacagcatca gtcattgaaa gtgcagctgt |
| 85501 | gtttttggaa tagagagctg atggaagtgc aaaggcacac tgatgagtat tatcctgaac |
| 85561 | aatggataca gatgcaaatc attatgcata atccataatg tccccatttt tgtagggttt |
| 85621 | tgtgctacgt gattgtgtgt gatttgatgt gctgagaact agaagtgtgc caaagtatca |
| 85681 | tttccgcttt cagaatagtt ttgttagaat tttgaaagat gagtaaatat accagaccgt |
| 85741 | tgcttttcaa ctctttgtag cattttgagt ggctaatggt tttgacagaa gaatgtggct |
| 85801 | gaatgataga ttgattgctc tgacttgata ttgttttcct tattccatta tgaaaagaca |
| 85861 | accagagatt ggatgtgaaa attccttaaa gaattacata ggatgagaaa ctgttctacc |
| 85921 | cactaagttt gccataggaa caagggacca ttctgtagag cactggtaaa gtaccagctt |
| 85981 | gcctgcattc aaaccgtggc tctgccacag atgagttgtg tgatcctggg gacattactt |
| 86041 | agctccttta tgactgtttc tacttttgca atgagggggt ggtaatgata gttttatcat |
| 86101 | acagagttgt tgtgaggatt aaatgagtta atatttgcaa atctttaata ctgtgcctgg |
| 86161 | catatttaag cattatatga attttacctg ccaccatttg tcacttatta ttgtattata |
| 86221 | cagtgtcata tcctttttct tggttattga aggatgaaat gggagatcag tcccaaatct |
| 86281 | gaatttattc tgcgatttgg caacattgaa aaatgttaag ccagaatact ttggttaaac |
| 86341 | catagcattt taaaaatcag gacgttatta taggagtact cattgtatct aaatctaaat |
| 86401 | ttatatttag tgctctggaa tttttcctca aaaatgattc atcgtttggc cattgagttc |
| 86461 | cccttttttg cagtttttaa attaagtctg actagagctt ctctgatttc tggtcccaaa |
| 86521 | gaattgcctc tgcctcgctt tatgatgtca aagaatgatg gtgagatacg atttgggaac |
| 86581 | ccagctgagc tgcatggaac taaggttcag attccatatt taactacaga aaaaaattcc |
| 86641 | ttcaaaagaa tggacgatga ggataaacag gtaaacagtt tgttcagatg tgttcatttg |
| 86701 | attctgacca tgaaaccgca tgtagcaggg cctcgtatct actgttcagc ttccagcaaa |
| 86761 | tctcaccatc tggaacccaa ttacagactt ggaagtgagc cagtaaagtt ttctcctagc |
| 86821 | aagaaagaga gaccgacaca tatgaatcac caccacccac ttaagggccc tttagagcct |
| 86881 | actttaaagt agaattgtgt ctaaaataag gctaccgatt taatacattt attgatttca |
| 86941 | ctagctcagt ggttttctaa atggattctt ggaaatgtaa cttccaggag tttttaatag |
| 87001 | gttctcttct ccaaaaagaa gtctgtagaa aaggtttctc ctattataat aaatgtgttt |
| 87061 | ggagaatgct gcttcatatt cccttgttga gggaaggact aaatcctcag tcttggaaat |
| 87121 | tcgtagtgca cattagcacg gtaaaagccc tgagaagttc tgcagtgaag agacctggaa |
| 87181 | atgtgtgttt actaaacttc tttcaccagg aaattctttc ttggggaaca tatttggaat |
| 87241 | actatcttgg gaaatgctgc cattacccat ttagcaggtt tggaaccaga acagggagga |
| 87301 | taggctttta aaattagacc actaattgca atggtcagat gatgattggt ggagacaaca |
| 87361 | gtataaaatc tcagaagagt gggcagatcc agaagaagta aatagtctac acattRgtgt |
| 87421 | accctgcagg catcaaggta tagctcatac ttccagcaaa gatactgaat catgcagaga |
| 87481 | agtcaaatta tgttcaacat agtgtcttct ataaggtaaa gaaatttatt ataggaacac |
| 87541 | ttgaggctgg gcacggtggc tcacgcctgt aatcccagca ctttgggagg ccaaggcggg |
| 87601 | tggatcacaa gaggtcagga gttcaagagc agcctggcca acatggcaaa accctgtctc |
| 87661 | tactagaaat acaaaaatta gccgggcgtg gtggcatgtg cctgtagtcc cagctacttg |
| 87721 | gaaggctgag gcaggagaat ggcttgaatc cgggaggcgg agattgcagt gagccgagat |
| 87781 | cacatcactg cactccagcc tgggccacag agcaagactg tgtctcaaaa aaaaaaaaaa |
| 87841 | aggaacactc gaatgaaaat gtttttaaaa tgcaatgaat attaaatgat tatgttggaa |
| 87901 | aactaacttc tgtgacttac cctgtgatgt tgtgtagatg aggtgttgct acagaagttt |
| 87961 | tatatggata gttaggggag gtgtagcaca tcctgttaaa caattccatg tactgtcact |
| 88021 | ttcataattc tgacacattt taaatcagtt cttcagggat atatttaaaa aggaagaggt |
| 88081 | ataggaattt tcagtcaata ttctttggca tattttccca ccagcatttg gagaggaatg |
| 88141 | agggtccact gtcccacggt ggttgatgcc ctgggaaatt tccaggccca gtccctgatt |
| 88201 | gtgtgaatga gaatacatcc tttcacttca ggctgagttt atgcatgccc cacccaatct |
| 88261 | atgggtgtga gctcatgctt tagagcaaac atattttgct ttttcttgaa gaagagtatt |
| 88321 | tgatcagtat ttcagattac atatttctct ttagactaag ggagcctata tatatagtct |
| 88381 | gtgtgttgat agaaagcatt gttttggctg ggtgtagtgg ctcatgcctg taatcccagc |
| 88441 | agttggggag gccgaagcag gaggattgct tgagcccaag aattcaagac cagcctgggc |
| 88501 | aacatagcaa gacctcattt caaaaaaaaa aaaaaaaaat tagctgtgtg tggtggcgca |
| 88561 | tgcctgtagt agctactggg ggtgctgagg tgggaggatt gcttgagccc aggaagtcaa |
| 88621 | ggctgcagtg agctatgatc acgccactgt gccccagcct gggtgacaga gcgagaccct |
| 88681 | gtctcaaaca aacaaacaaa caaaaaaaag cactgttttc ttggtacttg aatactccaa |
| 88741 | agttaccagt ctactagtaa agtttttttt tttttttttt ttttttaaag aggaaactca |
| 88801 | gcagcagata aggttgctta ctttgcctag ggttctacat caggttcata tttgaattga |
| 88861 | gattcaaatt gtagcttgtt aagatttcat cttagtgttt gggagtaagc tggttggaat |
| 88921 | ggtttaccta taatgaattg ttttgcagtc tctcattgtt aaaaaaaaaa agctagcatt |
| 88981 | ttactttgac atggtaagct ccaagaatat taagtattaa gcatttagaa atatatattt |
| 89041 | taatattcac atttgctgga agacttgata agtataaatc caggaaataa atttattaat |
| 89101 | gaatgaaaat gaaatctatc cacagagaga tcacaattgg gagcgggtat gaatgttatt |
| 89161 | tcatgtgatc agtgtctttc attttccaaa ggaaaatatt attggaataa ccaaaagtgt |
| 89221 | ttcaggctgg ttttagtaat ttaggtaact cagcactgat gagattttat tctctgggag |
| 89281 | acttYttgaa tcctcagaca ctgctgttta accatgctgc atttttgaga aaacatttcc |
| 89341 | ccccatttgt taaaccagct gggtcaccag cagtagagtt gcgttatgct tcctgactgc |
| 89401 | agctcagaac gccaggcttg agtgagcgcc tctgaactct gatctggttc tttgaagcag |
| 89461 | gaaacacagt ctcccacgat gtcacccctc gcctcccctc cttcttcccc gcctcattac |
| 89521 | cagagggtgc ccttgagcca tggctacagc aaactgcgga gcagcgcaga acagatgcat |
| 89581 | ccaggtaaga ggcgacctgg gggccagctg tcccaggcag tgtcttagag actccttaat |
| 89641 | ttaYcaggcg gtaacgcttc aagctgctta atttcctgat ttaattaccc ctgtctttgg |
| 89701 | aaacatggct caatttaatt ttatattctc aagcaaggtt tttttttaaa aattgattta |
| 89761 | aacatatttg agtaatgttt catctcctgt cctagaaact agttcgtctt tagacttgta |
| 89821 | aacagctaga tgatttaatc tttcatatac agattttatt gaatagaaca taccatatta |
| 89881 | agggcttgta ttttttttgg aattgttatt cctttctgta agcacaccaa agatatttat |
| 89941 | atagtaactg ctttaatgaa atttcttaag aaatataact gcaaaagcaa aagagttgat |
| 90001 | ccaacctttg aagactgagc acgtaaatga aaatatatag gtcctgtgct ttggaataag |
| 90061 | gataaaggaa atgctagggg tagcattttg ctgaggggta tgtagcatgc acagttgtgg |
| 90121 | ggttgccttc ttgctcacat ttatgctctg ctggcctcgt taggtggtaa aatacgttag |
| 90181 | ttctgaggat gacttgcaga gttggctgtt taggctgcag tgcctaaagc actggaaatt |
| 90241 | cactgcagca tcttgtgcct cctggggttc agactcctag tttaagatat gaccaattaa |
| 90301 | aggtattcta gaactggata tctgtaaacc cccacccccc cagttttgtt ttgtgggctg |
| 90361 | tggccccaaa tgacccagtt tctgcaggtt tttactgcat taaaaatagt ggtgactttt |
| 90421 | agagggacac agaaataaaa ctgcgcgcta ggctgaataa agagaggctg taatcttgca |
| 90481 | gagagaagca ggcgtggcat aatggcaata gcttttgatt ggaagcagaa ggtgtgtggg |
| 90541 | tttctgtgac tagctgtagg actttgggca ggccattgaa catctctgaa ccacgattac |
| 90601 | ctcttctctc ttaaaatgga gtaacagtat cctagagggg tattagaaga ttaagtgaga |
| 90661 | atgtgagtat aaaatctgtt tggaaattaa gtcctatgca catgcaagat gatgttgcta |
| 90721 | atggatagta acaaaaaaat cactgtcctg taacttgaat atctcagtgg ggttagaaac |
| 90781 | taaatggagt tgagaattct tcattttcct cttatagttt ccctgatctg atttccctat |
| 90841 | tttcaggagt gggttttttt tttttttttc ctatttctaa tcttacttgg gtttggaagt |
| 90901 | tggtttttaa ttcacaggtg aaactttttg ttctactgag tcagcctgtc agcaagttct |
| 90961 | acctttggta ttccttagaa aacttttctg ttcctcctcc ttaatgcccc attccctcct |
| 91021 | cattccatac aaattgggct tccatttccc cagtggtaag gaagcagctt gcttctctcc |
| 91081 | atctttcttg actctcagca gcctgtgcca aagctgtcca tgccttcatt tcttttcata |
| 91141 | cttttttaaa agtgaggtaa aatatacata tgataaaatg tacccgtttt atttgttcat |
| 91201 | ttttgagaca ggttctcact ttgtcactca ggctggagtg cactggcgcg atcatggctc |
| 91261 | actgtagcct tgatctccta ggtttaggtg atcctcccac ctcagcctcc tgagttggtg |
| 91321 | ggattacaga catgtgccac cacaccgggc taattttctt tttgtatttt tttgtagaga |
| 91381 | tgggatttcg ccatgttgcc caggctggtc tcgaacccct ggactcaagc gatccgctct |
| 91441 | ccttgtcctc ccaaagtact gggattacag gtgtgagcca caatgcctgg ccatgcacat |
| 91501 | gttttaaatc ttcaatttga gttttagcaa atggataggc ccttgtagct accactttga |
| 91561 | tcagtaattg aacatatcca ttatgccaga gatgaccctc tccggtcact ccctgccccc |
| 91621 | taacgcaatc actgttctga ttttcatcac catcattagt tttgcttctt ctagaacttc |
| 91681 | atatgaatga aaacacacag aatgactgac tgtactcata tgtgtttgcc atctttcctt |
| 91741 | cagcataatg gttgaaattt atctgtattg ttgtatcagt agttcattcc tttttattat |
| 91801 | ttagtagtgg gatatattgt gtgaatgcat cacagttcga cgatctgttc tcctggtgct |
| 91861 | ggacatttga gttgtttcca gtgtttggct attatgaata aggatactat gaacattcat |
| 91921 | gtatgagtcc ttctgtgaca tatgctgtca tttccctggc tcctaggaat agagtcactc |
| 91981 | agttttaggg tagttgtata tgaaactgcc aaagagttct ggtttgacac tgaacttcag |
| 92041 | agtgatgaaa tctttaggtt tttttttttt tttttttttt tgtacacctc cccttggaga |
| 92101 | aagatggtac ttccagttct tattaaacct cttgtaaggg gatcaaagaa ttatttgttt |
| 92161 | tgcactgtat tgcatgtttg gggtttcatt tgggaagaaa gagagttgag taatagctac |
| 92221 | tttttaacct tctgaattca gatccaatta aggaaggagc cagaagtcag acaattgatg |
| 92281 | gcctgaatat cagctttatt actgatatag ctgatcagag cacatgtctt agatttgtga |
| 92341 | ccagatgggc tttagacact cacctttcca gtcacaggca ggcttggcca tcccagattt |
| 92401 | cccacacaag taggaagaga aaatctgcct ctccaaaaaa ggccccccat gcagctgagt |
| 92461 | cttcatccaa gccctactga tgagataggt ctgtctcttc caagggaagg cccttgtagg |
| 92521 | tgagggatag aagccaaggg ccagatggtg gcctgttccc ccatgtgtct cccctggaag |
| 92581 | tagaagtaga agctcttatg ggaagcagag gcacgtggga ttcacttcct aaggaaagct |
| 92641 | catgacYgct agcaccccaa ccatgccaca gttcactgtg tttcaaacct tttggcagag |
| 92701 | cgcaattggt attagtgatt taattcagtg ctccatcaac acagaatgat tttaaaaatc |
| 92761 | ctacttctca gaagacaatg gtggcctacc agctgagact tactttttaa aatttggatt |
| 92821 | atgtgctctc tatgtatgta tgttttgcag tttttatatc tgtattcatt cttagcaggt |
| 92881 | tctgaaaatc tacttgatgg gttgccaggc aattgttttg ctattttaat gtgtgttaat |
| 92941 | gtattgtaac attgtgttcc agttactagg aagaaataca aaaagcagat gtgagcccaa |
| 93001 | gagtgattta tcattcttga tgtgctattg ttgtaccttc tttattttaa aaagagcaaa |
| 93061 | ctatgttaaa catgaggtca taacaggctg ctctgctctc tacctaattt tatttgtaag |
| 93121 | taagctacag aaataaaaca ttttacttat ttatatattt tcaaaaactt gaactattaa |
| 93181 | cctatagaga tgagaaagaa tttcaacttg ggtgataatt tctgctttct gttggagctg |
| 93241 | catactacct ggttatgttt aactgtctcc ctgccatatt ggtaaggtca ttattaagtc |
| 93301 | ttctggaaat aaaaggagtg aaaacataca ttcatttttg tggcttaaaa agttatttta |
| 93361 | aaggcagcaa aattgttttt taaaagtttg gttattgaat agtagaataa taaatataaa |
| 93421 | atatatttat tcattttcgt agtcttctct acatgatttt acattctgtt tgatttattt |
| 93481 | ttcaaaaata atgttggttc ttagtgttca aaatgcacat tagttttctt cttggggaag |
| 93541 | tggatgatca atggaagaca tttacttagg ggggatatat agaatttacc tcatatttag |
| 93601 | aatttgaaca cttatgacac ttttctagct attgttgctt tctatcccaa ttgaagttct |
| 93661 | cactgttcca ccttcacaat ttattcctaa tcataaccca gtgtaaatat ttttctgctt |
| 93721 | atcatgaatt ggttgaatgt ctttagattg tttagtgaaa ttgtgccaaa gagaatcagg |
| 93781 | ggttgtcagt cctagatggc cctatgagta ttctgttgcc agttctttcc tgaaaagcag |
| 93841 | atgctgtatc agtttgaatg tgaagtcata atttttgaat ggaatctttg cataccatac |
| 93901 | tgactttcct ttctgctgtt tcagcacctt atgaagctag gcagcccctt gtccagcccg |
| 93961 | agggatcctc atcagggggc ccaggaacca agccccttcg gcatcaggct tcccttatcc |
| 94021 | gggtaagtga tgatgcaggg ttatgccaat gggacagtag agtcaagctt tgggcttcgg |
| 94081 | ggacatggct ttttgatgga ccctttttca ttgcaaggaa gatataaaac tgatccttca |
| 94141 | tgcaaatatt gcatgtcttg tttggttgct tgtataaatt actaaatctt ttctactgtg |
| 94201 | tatgtctcta cgccttctca gtgtctctag aataaactag gctgcttctt gatctttagt |
| 94261 | atatctaaaa tgtccctctg ccttcaccta tctaatcatt agccaccact ttcaggagag |
| 94321 | cttcactttt tctaaagcag cccatcttgt ctctgcctta ttctttatta ttactcattt |
| 94381 | aatgcatagt taactgtttt ccatttgttt tgtgcctcta attacatggt aaacttttgg |
| 94441 | aaggcagaag tagttctgta ttctctctca gtgcctagac caagagaaca gttattgtac |
| 94501 | aatggcagca gctgctgttt ccttatgtaa taaatacaaa agaaatatta tggcaaaagc |
| 94561 | ttctggctat accacagctt agacaatttc agcttccctt tctgcagtgt tttcctttgg |
| 94621 | ataaagagct cttattttgt tctctgtctg cagctgttgc cttgtgtagt gtctctgggt |
| 94681 | cctgtgctgg ggaagttata ttctcaaaga agggtgtgtg tgtgtgtgtg tgtgtgtgtg |
| 94741 | tgcacgcgcg ggtgtgcagg tttactattt gacactaaag aggaagtggg gcagtttgta |
| 94801 | taacatttca gtcatctaac tcgggaataa ggaacatttt tgcacaggca cttctactca |
| 94861 | gaatagaagg gtggtgtcag acactcccat aaagatgtgg gctgctaggt tggaatagga |
| 94921 | taaacaaagt ctaaagaagg tcctggggtt catggaatac cttctgggga tggggcactg |
| 94981 | tgttagatgt ggtgcagaac ttgattttct aaggtgggac tctgtgtaca aataatttct |
| 95041 | taaagataat aatttagcca taggctattt tttatattta atgcttccta caaccataag |
| 95101 | atctagttac tgctgcttct aatagtataa gtgagttgac tgtggttgag agaggtcaaa |
| 95161 | tagctggttt aaggtttcat tcataactag tcagtgacag agccagaatt tgaatccagg |
| 95221 | cctgtccaat gtcaaaacct gctcctttca ttactttgtc ctacattaga gtcccatatt |
| 95281 | acaagaaggc taggttctag agttgctaca taaagaaacg ttgggtgtag taaaaaatac |
| 95341 | tcttgaaaaa tcccttattg ataccacact ggagaaccaa tatgccattt gctcccaggg |
| 95401 | ccagtacagc tcagttttat ccccaagttg gaaaacacat ttgtttcttc agctgagctc |
| 95461 | cagatgtagt agttggctgt gtttagagat cctgtggtac aaaaatatgt gcctttaaac |
| 95521 | acgagaatca cacccactgt ggagtgaggt ttacgttaga aaaggtatca gtaggactgg |
| 95581 | gatttatccc taagctctcc aaaatgtggc tgtgtaggtt cctacagagt gtgtcctggg |
| 95641 | aatacagagg ccagatcctg aatggttagg atgatagtca aactaccagt gcatactgag |
| 95701 | ctttacagca gtgtgactgg gctcactaga ccctttctac ctagcccctg gctggcatgg |
| 95761 | gcatggatgc tcatttggat tggcaccctg tggcttctct tctaatctcc atgttccctt |
| 95821 | ttctgctgac aaacatgctt catgagccct tcactcaact aatatttagt gaacacctat |
| 95881 | taagtgccag gcactgtgtt gagaatacaa aaatgaggta taacactaac agtttctgcc |
| 95941 | ctcatggagc tactggagag atggtcatta accaaaccac cacaaagtaa atgtataatt |
| 96001 | atgatatggg atatcagtag gaattacact gtactttaag gatgaaaatg ggcatggggt |
| 96061 | atgacctagc cttgaggggc aggggctgat caaagaaggc ttctggtggg ggagcaacat |
| 96121 | tcgagttgga ataagggtac atgggtgtta tccaggtgac agggtgggag tgcgggagca |
| 96181 | aggtgacaag atggagggaa ccgcatatac aaagacattt cctatgggtt tgccatttta |
| 96241 | tgaacattat ctctttcagt gcccacagct atcttatgaa ataattagtg ttgttatccc |
| 96301 | attttcagat gtcagaacct gaggtttaga gaccttattt aatttgccca agttcacagc |
| 96361 | taggaagtgg cagggctgga gttcaaacac tggtcaaggt tcctcagtct ctggctcagg |
| 96421 | aaccagatag atggtggtgt cacctgttga gctaggaagc cctagatgaa atactggcac |
| 96481 | tggggaaaga tcacaggttc agtgtttgat gtcaggaact gaggttttga gatagtccag |
| 96541 | agaagctgtt gaagaactgg tagattacat gggtctggag cttgggagag agcttaggct |
| 96601 | ggacataggt atttggcagc aattggctat cagaaggtct catggtatag tgcagaaaaa |
| 96661 | accagacagg tgctcgccca agtccagcaa aactctgtgt gaccttgggc aagttccttc |
| 96721 | tcttttctgt aatacaaatg acttcgaatg gatttaacag cccattgtaa caatcagcaa |
| 96781 | cagtccgatt ctcttaatgt gtctgtggtt ttcatggctg gtgtgttttt ctttcctctg |
| 96841 | ttcttaggtt tccttcttat gcctgttaac ttggtttggc tggactccgt gttccagaaa |
| 96901 | gcaaaatgcc aacctggatt agtcatgtag atgaggggat ttgaagtgaa ggttcttggt |
| 96961 | gataagggat taatgtttac tgggcatggt gctaggccta ggctggggtg actaatcccc |
| 97021 | ctcatgttgt cacgttgatg tgccgatggc aaccttcttt ctcttctaac tagttctctg |
| 97081 | gccttgtatt caagttcttc ctgcctgatt acaagggggc atgtcctctg ggtcctgggg |
| 97141 | gaagccttta gggactccca gccagtgctg ttgcaaccca tggtgttggc attttagagg |
| 97201 | tgcttatgcc aatccagtgc ctaaacattg gaatgatttg tctttttgct tactatttta |
| 97261 | ggaatttcac ttttgttcgg gtgttggatt tttttccctt tgaaatttca gacactaagc |
| 97321 | atatcctatg ttggtagatg attgatagtg ctttgtaaag aaattataag acttattttc |
| 97381 | tgcttcatct gttatcatat gtttagatgt tgttttgtgg tttgtttttc acaaattacc |
| 97441 | ctcaacatct agatgatgta gtcatttgta attttgctgg caaaagattt agaattgcac |
| 97501 | gctttattcc tactgttctc agctgcctct gtttttgcca gggtcatgag aaactttatg |
| 97561 | ttaagccgtt gtctttattg tctccttggc agctgtggac actgaccatc ttctcgaaat |
| 97621 | gctctcatca tttggtgtcc attatgcctg ctctcctggt tttcctcatg cttctatggc |
| 97681 | tgcccctgcc tcatttgttt caatcctcct tcctctgccc tccccttgtt atgctggaga |
| 97741 | ctgctgggcc tacttctcac tctacttact ctttagatgt gtggatgctc atgaccacat |
| 97801 | agcaccatct gccagacatc ttcactctaa tgttccactg tcactttcag ctgacatgtc |
| 97861 | tttctcataa aatcagcttt cactcttcca ctcctagctc cacccaccca tcagcacagg |
| 97921 | ccagaatgct ggtcattgtc tttgacttct ctttctctcc ttatgcctgt tcagtcccca |
| 97981 | agtcctgacc ataccacttt tcaaatagct tgtgtctgtc catgcctctg tacttcttcg |
| 98041 | ttcaggccat tatccttttc actgattgta ttgtattagc ctctctcttg tttcttcttg |
| 98101 | cttccaccct tgccttcttc caattcatta ttctcattgc acacaaaaac aatcttaaaa |
| 98161 | acacaaatct aatcatcctg ctgacttgat tgaagcctgt ccctctctgc ccatggctct |
| 98221 | taacatgtca tccatgttgc tgtctggccc tcgcctacct cctcggccac agcagctcac |
| 98281 | tgccgctttc cctccactct ctggccattt cccggatggt cagtgttctc tgcgtgggag |
| 98341 | ccttactctt caccctcact tttgccttct ttgttttctc ctttgtctca attcatctat |
| 98401 | gcttttttcc ccattaagaa cgtgaaacgt aagtgttttc tgatattttc ttgtatatgc |
| 98461 | gatgtttcct ttttttttca ttcttaaaaa gaaaaagatc tattgcttgt cagctcattt |
| 98521 | atgtgtgcaa ctcagccaga gggatgggcg tctgatcatt attggtttcc catctcagag |
| 98581 | gcaaccaggg ttgcctgttt cttttgtatc attctaggaa cattttacgc atgtacatgc |
| 98641 | aaatataggt attcatatat attaatatgt gtgtgtatgc actatatgta tttatatatt |
| 98701 | tatttatttt tttagacgga gttttgctct tgttgcccag gctggagtac aatggcacca |
| 98761 | tcttggctca ctgcaacctc cacctcctgg gtccaagcga ttctcctgcc tcagcctccc |
| 98821 | gagtagctgg gattacagge attctccacc atgcccagct aattttgtat ttttattaga |
| 98881 | gacggggttt ctccatgttg gtcaggctgg tcttgaactc ctgacctcag gtgatctgcc |
| 98941 | cgcctcggac tcccaaagtg gtgcaatata tttttacctg aatttaggga gacatgtttg |
| 99001 | gcaaatgaat atattcttcc agctagattg cactagaaaa aattatcttt tatttaaaac |
| 99061 | caaaacacat tcacttttca aaccctcagg atactgttgt gatgttttct ttgctttttg |
| 99121 | gagggatgaa agtaacagtt ttctagaaca tattctcaac actcactact cttcttcaga |
| 99181 | gatgaaaaaa tgacaaacag aatttagata cctctattac tctaccattc ctcagggggc |
| 99241 | ttagctatat ttttggtgta agttttctaa tgatcaggta ggggagggga tgttaggcat |
| 99301 | tgttagccag caccatattc atctcgaagt gcataacttg aaaaatgttt atttaactta |
| 99361 | gttatctcct ttcctcctca tctcagtttc ttgccgtacc tcctccccta gatataactg |
| 99421 | caggtactta cggccagaag caggtgatct ccactccttg ggtcctcttg gaacccaaga |
| 99481 | gaaaaggcaa gtatatgaaa gatgattaaa gaaatgaata ttcctgaggc tctgtcagag |
| 99541 | tggtgttggc ctgagtcaaa agataattga tagtcaccac aaataaattg ttcattctaa |
| 99601 | gagctttgat gaaaacagca ggaaatagcc tttgggggag gctgatattg caccgagaga |
| 99661 | tcttcttgtg gcatgaaaga tattttatgg gtagactgga ccaggatgca agcaggataa |
| 99721 | ggagagcgga ggagtgagaa tgactgatgg tttgggatgg gattgagtga ggtgggttag |
| 99781 | agttgagtca tgagagagtt ggtcatctga gtttggaaga agagtgaagc aggtaaacaa |
| 99841 | gcggggcata ctgggtactg tcttggctac tctggttgtc taacctaata ctcagcccag |
| 99901 | ggaattgacc cacagagcag gtcaggcccg cccggattct tttggggttc aagaattatt |
| 99961 | taccaaggca ttacttggta agtaagtgtt catacgaacg tgtgtgagct ctcaaacaca |
| 100021 | cttgttcttc agagcaaata cagattttaa aaatatgtca aacattcaac agatacaaaa |
| 100081 | gggcacagtg aaaactgtcc ctaccatttc cccgcacagc cactcatccc aggggcaact |
| 100141 | gatatgaccc atttattttg tatagtacta tacaaaacat ttatataata ttgtatatga |
| 100201 | atatcttttt tatatacaag tgaaaatagg tattatatat aattacatgt aatatatgca |
| 100261 | aaacacagtt atgctttttt acacatatgg tggcatacta tacatctaac atgcatttag |
| 100321 | cactaacaat aagctttatt aacgttctat agcagcacat aaagtacttc ctcatctttt |
| 100381 | tgttttttac agatgcatag tatttccttg tctgcgtctt gtgtaaatca tttagccagc |
| 100441 | cccttttgat tgacatcttg tacttgcaat cactgcgttg tttgcagata tatgggagac |
| 100501 | caattcctag aaattgaatt attgggtgaa aatgcctata tatttgtaat ttttgataac |
| 100561 | gactttcaaa ttggaggtat agcagcctat gtacttgcca acaatatatg aaaatgtctg |
| 100621 | ttttctcgta cctttgtgaa aacaatgtgc taacaaattt atgggtcttt gccaatctga |
| 100681 | taggtgaaaa atgatagctc actgtagttt aattagcata tctctttatt agtgaagtga |
| 100741 | aatatcttaa gtttatgagt cacttgtatt tccttttctg tgaactgttg attcatatca |
| 100801 | agtgcccctt tttcttttga gtattggttt gtcattctta atggcatgaa accagaagct |
| 100861 | ggtctgtctt ttcctttgga ccagagatat actggagtgt gtgtggaaga tttctccctg |
| 100921 | agtggtgaga ggagatgctg gcacctgagg ctctcgaagg aggttatgga ccccagctgc |
| 100981 | ttcccagttc tggagaaccc caggcctgcc tgcagtgcag atttcttgtt tataaggtca |
| 101041 | tgacagtaaa gttgaattac ttagtttatg ggtagagatt agggctaaaa aaaaatcctc |
| 101101 | tgttgttaga gatggtaaat gtcaacactg tgtgtgtgcg ttgatctctc agcattgctt |
| 101161 | agacagctaa attatgttcc tttcactgaa atgggtggga ggaggagata gaggtagaga |
| 101221 | taaagataga tagagggaga gagtataagt gtgtgtgtgt gtgtgtgtta ggaagagtga |
| 101281 | gtttgatgcc ttttctaggt atgtatgtgc cttattcttg agatcttctt tcaccaaatt |
| 101341 | cattcctcgt ttcatttgag accccagctg gacgtttata ccatctccta aggggtcaaa |
| 101401 | ggttaacatg tcctccaggt gtcctacttc actgagtcta ctatagaacc cacatatacc |
| 101461 | tgaacggaca ctattgccac aaggaggtcc tattttttca tagcctccag tctcctgctt |
| 101521 | acttattttc ttcacctgct tgtagttctc tttctgattt tctagccctt aacttttgag |
| 101581 | gaaggatttc tagctcttaa cttttgggga aggaaacatt taatctcaac tgatactgaa |
| 101641 | gtttattttc tcagtgccaa tcttttctac ttgtttcttt tttctttttt ccttttcttt |
| 101701 | tcattgattg attgattgat tgattgagat ggagtctcgc tctgttgccc aggctggagt |
| 101761 | gcagtggcgc gatcttggct gactacaacc tctgcctccc aggctccagc aattctcctg |
| 101821 | cctcagcttc ctgagtagct gggactacag gcgtgcgcca ctgcgcctgg ctattttttg |
| 101881 | tatatatttc ttttagtaga gacggggttt caccatgttg gccagggtgg tcttgaactc |
| 101941 | ctgacctcaa gtgattcacc cgccttggcc tcccaaagta ctaggattac aggcgtgagc |
| 102001 | caccatgccc ggcctctacc tgtttcttag tgtattcctc aggtgctcac tgtgtaccac |
| 102061 | acttcttgac aataaaagag atgtttagtt aatttagcct cccagaggta gctgaaaaca |
| 102121 | ctggtttgaa ctgctggttc caggcagtac tttgtaaatc tggaaaggat agtagcttat |
| 102181 | tccccaggac cttttggtct cagttatatg ccaggctatg gatatggaag acttctttac |
| 102241 | aactggcacc attttagccc ctgggcccat cagtctttcc cttgatctca ggcRagcagg |
| 102301 | gagtgtcatg cccccccagt tgcctatccc aacaatagtc actactgact gaggtgcttc |
| 102361 | tttggtccca gtgctgtgct ggcccttttc agtcctccca acaatctcac aaggtaggta |
| 102421 | ttattacatc ttccatttgt ccatttggaa atacttaagt ggcttccatg agtcagccac |
| 102481 | catgccaggg gtacactgag gagtacacct atagcctctt gctctctagg tgcttgcagt |
| 102541 | caaaaggagg agatagattt gaatcaaatg atcccaccta tgcatgtgga attaaaaact |
| 102601 | gacaattgcc ctaaaggcct taaaacatgg taccatgtag caaaggaatc cagcctatag |
| 102661 | ctagaaagtg agagaaggct cctttaacaa agtatgtgtt gagctgagct gtgaaggatg |
| 102721 | aatagaagtt aaccaggcta aggaaggatg gtcctgggag gtgaggagag gagtggagtg |
| 102781 | tttcctgtag aggtaagagc ctctccaaaa ccgcacagca gagctgggct gctgagagag |
| 102841 | ctttgcttct gtcacaatca gaaagctaca gcattgggaa actgctgccc tagtgtcagc |
| 102901 | tccaaccaga tccacactac aaactacctt ggcagccaaa tgaaagcttt gttccctgcc |
| 102961 | tgtaacttgg gtctgcatcc gtcttccgtg ggtgtatttg ttgagtggaa cctaagtcct |
| 103021 | atttggaatt ctagcagcaa aggtgttttg acagtggagt ttttagctag Ycagcttgtg |
| 103081 | cagtgctggg acttatgctg acgggtggaa tggactcgtg acccagtctc taccatattt |
| 103141 | cacagggaga gcagatacat ttgaagaact gaaagaaggc agctgtggct agagcctgga |
| 103201 | gtgggaggga gatgaggtcg gagagggagg cagggccctg tgggctatgt gactgccttg |
| 103261 | caggtgggag ctgatggctc agaagttaat ttactggtta gtaagtgacc atgccaaatt |
| 103321 | ggaacccaag tctgtctgcc tctggaacct gtactctttt tgcacctcca gtcagtagcc |
| 103381 | tcattctcca aagcattctt gaattctgtc caagttgtgg atgcatctgt ttcccatctg |
| 103441 | aactcagact gccttgagtg tgaaggggag aacgtagtga agagacaggc agccttggcc |
| 103501 | tgaccagcaa ggacaggggt gggaacgtca tcgcttcgtt aatggaagtc tgcagccttg |
| 103561 | tcagagtctc ctcttactgc tgatttctca tttttcttgg ctttctcctc taagcagagc |
| 103621 | acaaaatcct gataatagtg aaggaaaagt gcccttctgg aacagcaaat cttggcccct |
| 103681 | gaggggagaa ttggaggcag tggttactaa tgagcatttc ttatgttaat caaaatactg |
| 103741 | ttagcgccac atactgcaat cacagtaaaa tgattacata cctggattgt atggctagct |
| 103801 | ctaggattct tttgtgatga cgtgccagat gcttacctgc aaaaattatg tgagaaataa |
| 103861 | aatggtgctc tgtcagtatg atttaatagt tcgccagcca aaatctaaaa ctaaactaga |
| 103921 | tgatggggag gaaggtgtaa tacagcatag tcatagctca ageacagate ctcaatctgc |
| 103981 | aaaattgcag caatgattta ggcattgcag tgaggttctt tttgtaataa atgctctatt |
| 104041 | tttacaggat ggatgaacag atagagataa ctatagagct tgatgatcaa gtgtacgtac |
| 104101 | tttggaccca gtttgtattc tggctctacc atgtgccata ggtgtgtggt cttggtaaat |
| 104161 | tacttccctt acctaatcct cagtttcctg ttctgcaaaa tgggaattac cggatttacc |
| 104221 | tcgcagggtt gtgaagatga aaattgattg tgtgcattag gtacttacag tgcctggcac |
| 104281 | ttagcaaagc aatgaatgct tgttacctga cattcttctg tgtctaagtg atcgtttcaa |
| 104341 | aaagtatagc ttattgtaat aagcagcaac tgtgtgttgc acattaggct cagagagatt |
| 104401 | cctacagttt ggcagtcagc cttctgaact attgaaatta gatccctaag taagttaggt |
| 104461 | aatatgtggt taaaggcatc tttgtgggtt tatttccctg aagaaacaga taaggttaaa |
| 104521 | tttctctgtt gggacttaat tttaactaac ttctctacag aaaaaccaaa ttatttattg |
| 104581 | caacttcctg tgaatctata attattttga aataaagact agaaagaagg agttttttaa |
| 104641 | aaagttctct gacttcagag gtaagggaga gggctcagcc tttatagtgt tggatgacat |
| 104701 | tatgtgataa atgcatataa agttcagctt cagtcagctt aagagaactc aggttaacct |
| 104761 | ttctctgtct ctagactggg attgagaatt ttttcgaatg tttgtttgtt tactcattca |
| 104821 | gcaaataact tattgagtgt tttgtatgtg ttagtgactt ttccaggcat ttgggatata |
| 104881 | gcagtgatca ggatagacaa aattttctgt cctcctgggt tttatgggat agcatggggt |
| 104941 | agggtgagag gcagagcata aacaagattg atcaagcata atttggaatc gtttcatgtt |
| 105001 | aaaacaaaat tgctaagctc tgtctccttt tctaggtcct tttcttcatt tctcattgaa |
| 105061 | cactttcccc acaattttac ttgagctgtc ttttcttttt aactgggaat ctccttcaaa |
| 105121 | tccatggaag atgaacctat attactcaaa cacagaaaga ctactataaa aatgaatcag |
| 105181 | catcctggtc ctatagattg tctgcataga taatcaaatt agaatgagaa agttgtttgg |
| 105241 | gctcataatg gtctaatgtc attggtcaga ttgcaaaacc acaatagctt caggcttcca |
| 105301 | tctccaggat gggctttgtg gttgtgaagg gcagatcttc ctttcctttg ggtcttgctt |
| 105361 | gaaggataca ttcattgagg gaacagctgc tataacaaat aggccccaca ttttaatgac |
| 105421 | tttgcacagt aggtatattt ctcactaata taatagtttg gcaggccgct gtcctcagca |
| 105481 | tggtgattca tgcaccagag ctccttctgt cctgtcactg tgccatcgtc tgttcagtgg |
| 105541 | gcacatagga aagagagtag aggagtgcta cagggtacta gtcctgcttg gaagtagggt |
| 105601 | aggccactgg tgctcacatt gcaatggcta caactcagtc acatggctgc atgtaacttc |
| 105661 | aagggaggct ggaaaatgtc atttctggtt gggcagccac ttagtgacac etetacacca |
| 105721 | ggcatagtag agcatgaatt attggaggag tctgctgtaa tcccacctat cagcactgcc |
| 105781 | accacttgct tctgtgactt ttttttctca gccccatgac tgaccaattt ccttgatctt |
| 105841 | tctttagaca aattgatatt cacttgggct atagtgtagt ctcaccagcc cattagctat |
| 105901 | ccttttcttt acagtagtca agtcttctca catctttgct aatgagatcg ttttgatcag |
| 105961 | caattgcctt gtggtagtct tatttccaaa atatttttgg cggaggaggc atcctcagat |
| 106021 | attcactgtg ggcttagtat gagcttttgc tactccaaat agtttgttta ctaaggcact |
| 106081 | aacaggacac tgttaaaaga tcattacccc tcataggtga aatggctgga ctctaaaaca |
| 106141 | gcatttatag gatgccttcg gggtaggttt atattttaaa cagaaatgtt ctatgattga |
| 106201 | aacaatacac acacccttct taagttctct ccaacaatgc ttccttgcaa ttcatattat |
| 106261 | ttagctttgt ttgattttta agatagtttg atagtagggg taaatgttac cttttgggat |
| 106321 | tttattaata gtattacttt ttaatgagtc ttttgaagtc tttcattata aacagcctgc |
| 106381 | tttccccaga gataataact tgccatgcca aatatttgca tttcacttga aacaaaacca |
| 106441 | aggggttgaa tgttttaaat ataagcaaat ctaattctcc tgtgtctaga gaaactgtat |
| 106501 | aatgaaccat atgtatgtgg cttaagcaga cagatgtgaa agatttctcc ctctctcctt |
| 106561 | ttatttaaaa aacaaaacac tggcagagct caaaattggc accaagtagc aaaYatctct |
| 106621 | gctcagccaa caaaaccact ggagctggag ttgatggggg gaggcctctt tagggtgcat |
| 106681 | gcaaaagtgg agcttttcct ggtcctggct tctacttgga aagctaaggc tccacagctt |
| 106741 | atttgccctc atttctcagt gccttcaggc ttcaaaatga gccattctca ggtgactgac |
| 106801 | ttttcagatc tttgagaaga ttgttgtggt tgattgttgg actggttcag aagacatggg |
| 106861 | tccacgcatc tgggtcctgc ggtgacatgg cctctagctg ctgacagagt ggtgctgagc |
| 106921 | taccaaacag actcataggc ttcctttcac aaagaacatc atagggcagt gtttccacat |
| 106981 | ctcttgcaat acaagacata cctctgaact tcctcaagct tgccaataag tagagctcac |
| 107041 | actacctttt agtatctgtg atgtacctgc cagggactat aggtgggata aagtatctga |
| 107101 | tatggctcct gtccttcaca ttgaaacctg ggtgaagatg tttaggaaca ctttaaatgt |
| 107161 | tcaaactgtg gtacttacca tgactatcat aaagattcat atatattttt ttgagacagg |
| 107221 | gtcttgctct Rtcacccagg ctggggttca gtggtacgat catggctcac tgcagccttg |
| 107281 | acctcctgcg ctcaggtgac tctcgcacct cagcctcctg agtagctgga actacaagtg |
| 107341 | catgccacca tgcttggcag tttttaaata ttttttgtag agatggggat ttgccatgtt |
| 107401 | gcccaggctg gtctcaaact cctagagtca agtggtctgc ctgccttggc ctcccaaagt |
| 107461 | gctgagatta tgggtatgag ccaacatgcc cagccaagat tcatattttt taagacgctc |
| 107521 | aaatcacttg gcttagtaaa tcaatttcaa gcatacagcg agaagcaagg ggaaagagat |
| 107581 | gggggggtag gttagcacac ttagtgcaaa ccagtagaag ggccactcta tgggttacac |
| 107641 | agaactcatg tgccctctcc ctttgtccta aggcctttct aggtttagga gtaggaactc |
| 107701 | tccttgccca cttaagtttt cttggaagag gggttgggtt ctcatgggaa tctaagaaat |
| 107761 | gagagccaag gaatggccca ctggtctgta ggggtgggag ttcttggctg ttcatgcttg |
| 107821 | ttctactgtt aacaggcctt gccctggttg tgttaagttt gctccactgt ccacttatct |
| 107881 | ttaggcttct gcagcctttc ttgccccccc ccccggcttt gtgtcctttc tgtacctacc |
| 107941 | tcccagcttg tgctgatgct tggttctggc gccctcataa ctttaagtcc cacctaccct |
| 108001 | ttctctatac tacagcccca ctgagcatct gtttagggtc tactttctct tctcattggc |
| 108061 | cttgatattt ctcatccaca ttcccaagaa tgagcatctg acctaagccc ttgtcagagc |
| 108121 | atctcaaagg ccagtatact gtgtgcccat gaacccttac tcccactcag gtgacgccag |
| 108181 | gtgtgaggtg ggtcatggtg cccacacaag gctgccaggc ccacctttgc agcagagcat |
| 108241 | gggaacgagc agtgccgtct gtcatggccg tgggtacggc aggtacattg gaggatttga |
| 108301 | gcaggggaag agtgttctgg gaagaagaca aacatcttac ccctgaggag tgagcatgtg |
| 108361 | cagtcggaaa gcctgctgag aggaggctgg ggtgcgaggc ttctgaaggc caggtggagg |
| 108421 | aggtggactt ttctgttagg cagcaggtca tctctgcagg cttttaagca gaggagttaa |
| 108481 | tcaccccctt ctgttcattc ctgatttctg ttgttagagt agacctgaga cctgcctgct |
| 108541 | aagaggccac taatgccacg ctggagtgag gtgatgtcaa gaggagctag aaagcagatg |
| 108601 | gttatggcat tgaggaaaga gcacacatgg gctgacaggg acagctaagc agcagcttca |
| 108661 | tgagcaggag ggaaagggcg tgttctaggg gagcagcagc ttccatgtca gactcaggaa |
| 108721 | tgcagcagac ccaacatcca gcttggagat ttcttctctt tttctttttt ttcaatggaa |
| 108781 | ctgtattttc gcaaaatgtt acagatcact tatagcaaac agaatggtga tggtcaagga |
| 108841 | aactgactct gggctccttt ttgaccgcag cagctatgtg gaagcagctg cagctgtgat |
| 108901 | aagggccgcc cagcatggat atttctgtgt cttcgttcac tgggaagtgg agggacagag |
| 108961 | cttccgaccc acatcccacc ttcttcacga atcatctttg ccaccaaatc gttggctcat |
| 109021 | aggtgtctgt tcttgtcttt cctgcccatg cctggctttt aggttccttt gctccRgctc |
| 109081 | tttctgtcag ggctgacctg gagggcagtg ctcctgtgag ttctccacat tatatcatgg |
| 109141 | ccggtaatct aatccaattc aagctgacat tctccaccct atgttctacc aatggaacct |
| 109201 | ggtcagacat ctgggctcat tcaggaagtg ctgctagggc atggctctgg aatttggtgg |
| 109261 | gagtattttg gaaggttgtg tggtctgttt ggttgtttag tgctcggact ggattttgat |
| 109321 | tttttttgct ttacttattt ttactgagac ggagtttcgc tcttgttgcc caggctggag |
| 109381 | tgcaatggcg cgatctcggc tcgctgcaac gtccacctcc caggttcaag tgattctcct |
| 109441 | gcctcagcct cccgagtagc tgggattaca ggcgcctgcc accacacctg gctaattttt |
| 109501 | tgtatttttt tagtagagaY ggggtttcac catgttggct aggttggtct tgaactcctg |
| 109561 | acctcaggtg atccacccac ctcagcctcc caaagttctg ggattacacg tgtgaaccac |
| 109621 | tgctctgggc ctgtttattt ttataataat aagaggtttt tgaaaaaaat tcagatagat |
| 109681 | gcaacccagt taaaaaaaat cccggtatac aaaatcctgc ctttgaataa gagattaaaa |
| 109741 | aaagatggtc attatttgct gcctgcaggg gttcactaag ggatatgggt aagttccctt |
| 109801 | agctcggaaa aaggataata attaataccc gcttacatct ccaaagtctt gaggtgctca |
| 109861 | aacaagtagg agtgtgtgaa atgctttgta catatcaaaa cactctgtga aatttaaggt |
| 109921 | attaactaat ggataatgac ttcttgaaca tttaagagtg gtttgattta cgtttagtag |
| 109981 | aaactcatct agtatgaaaa tgatacattt attacaaatg attaacaacc ctacagctct |
| 110041 | caacctcttt atgtaacctt ccttctgact aaaggaccca gatgactttg aggaatagcc |
| 110101 | tgtttgctgg gtgcattctc actcagcact tctggaagta tccattctac agttagtatt |
| 110161 | tcatctcatt gtcctggcct tccgaaagca cccttaaccc aaggtgtgag atatcaaagt |
| 110221 | aatacatatt ttacttcaga gatgacatct caaggtttta cagtagctca acattttcta |
| 110281 | gtctcctttc tctgaacatt gtcagagggg agacaggcgt tcctgggaag aattaattcc |
| 110341 | tggaatggtg tgtgtgtgtg ggtgtgggga atgggaggag atggccggcc ttcttttcta |
| 110401 | gggttagtgg gtggaattgc ccgcccaatg attaattccc tccttgacag tgtgttcaga |
| 110461 | agcaggcacc ttgatggctc tttagcagca ataccctcct cttctttcct agcctccttt |
| 110521 | tgtcataaag cctcatcttt tttatttatt tttatttatt tttttttttt tgagacagag |
| 110581 | tctcactctg ttgcccaggc tggagtgcag tggtgcgacc tctgctccca ggttcaactt |
| 110641 | ctgcctccca ggttcaacct ctgcctccca ggttcaagtg attctcttgc cttagcctcc |
| 110701 | cgagtagctg ggattacagg cacgcaccac catgcctagc taatttttgt atttttagta |
| 110761 | gagatggggt ttcatcatgt cggccaggct ggtcttgaac tcctgacctc gtgatccacc |
| 110821 | tgcctcggcc tcccaaagtg ctgggattac ttgcgtgagc cactgcaccc ggccaagcct |
| 110881 | catccttctg gtgttcacct cttggtcctg ttaaggtggc ccttggcaag tacctcttta |
| 110941 | gtcttagggt tcatgggagg gatagaacaa aggcccacgc ctccccctag catcgacatt |
| 111001 | tcccttcgtc tgccaatgcc taccatagct ttgttttctc ccgcactgct tccctggttt |
| 111061 | tctaggctct taaatcctca aatttgactt gcatgccctc tagtggcagg tctgatgcac |
| 111121 | cccttgttac cctgtttatt gtggtaccat aacagctgtt ctcaccaccc agctctgtta |
| 111181 | ctaaatattg tctgtaattc acaaatgccR aatgagaggt tcagtggtcc tgacttgcca |
| 111241 | ctcaagtagg agctggaaat gtgggatttg aattggagct tttgattcct ggatttatac |
| 111301 | agcatttcag tgtaacggca acagtaaaaa agatgtggtt atagttgttc tagtttcttt |
| 111361 | tttgtcattt aaaaagaatc cagattattt tactatccac tcagctaatc tgaaagaatg |
| 111421 | aaaatgtgag gaatagtgga aaataagaat gatcatcttt ttttaaaccc tttattacaa |
| 111481 | ttagaaattt ttttgtcact tactgagtat tcagtatttc atgcctcagt cctgttaact |
| 111541 | ttgaagaata caaaaaatac ataaagcatg atgtctgccc ctaagtttct gttctagtta |
| 111601 | gattctagtt atgaggcagt taaatttctt cacctgagga aaaaaattaa gcataacaca |
| 111661 | tgataacttt tattggtgtt taatttttct acttctcact aaagacaggt tctctgagca |
| 111721 | tttacttttt aatttattct tacagtcctt ttcagtggag agggaactac aggataacag |
| 111781 | cagttacccc gacgaaccct ggaggataac agaagaacag cgcgagtact atgtcaatca |
| 111841 | gttccgatcc cttcagccag acccaagctc tttcatttca ggtaagaatg tgggctccct |
| 111901 | aggcatcact ctcaccagaa catagccacc atcttgagaa atcattcacg caaaatgagt |
| 111961 | atggcagcct tctgtgattt cagtttgttt ttcatctaaa gtcgaatttc ttatcagatg |
| 112021 | taaaccatgc agaagaaaag ttgccaatga cgtttggaaa attatatttc catttcaaag |
| 112081 | ggcttaagtg ggaatttggg ggcaacttgg cccagaccct gcaccttatc agctgaggca |
| 112141 | gttcttgaaa tgctagcgtg tcgccccaag gggccacctc tcattgcttc ctcgttatat |
| 112201 | tagagctcgg gcaaggacag aatgtggcct cctaaggagt tggtgtcaca agtcttttta |
| 112261 | tgttatatgt gccttcaaac ttcaaactca ggctcgtttt ctttggctca aatgtttgtc |
| 112321 | ctccagtttt tctgagagaa attaaaatgg gggaggaaaa ttggccagtg ctttcccact |
| 112381 | tgtcagttct tctgggttgt cttcagatat atagattcct gttgagttct catgtaaaat |
| 112441 | tcaagctcag aggaactggt ttaatgccag tgtggctaca ttttaccagg ctgaaagact |
| 112501 | aacagtgaaa tagtagagtc tagaagaaaa attcataaag ctttcttttt agactttaaa |
| 112561 | atttgaagaa taacaatatg tccttaataa ttgtaacctg ccatattgaa gtgtgggcaa |
| 112621 | agattagttt tatcttgtga aatgaggcac ataattgaaa tatacttgta taagtaattg |
| 112681 | caaacatcat ttactgattg gtggtaatgg ctttattgac ggcagaatag tacagataca |
| 112741 | acaaattaat gttgacgtca tgattaagga aaataagcag aaacccatat tgcacgattt |
| 112801 | gaccacttgc taaccttacc ccgtgctcag gtgagtttct ttacgacatg tgcagatatt |
| 112861 | tagccaaata caaacaggta cttgttagaa atgtttttgt tttcttttaa cttttgtatc |
| 112921 | ttgcagaatt tcagatatag acacaactag tctggctgat acaacaaacc tctgtgtacc |
| 112981 | tatcacatag ccccaatgat tttctcttac ctacagttaa cacataaggt tgtttcagtc |
| 113041 | agtcaatgat ggcggtacca taacagtatc acggagctga aaaattccag ctccattaat |
| 113101 | cacctagtga tgctgtagcc atcatgatgt tgtagcacaa ggcagtgctc acataaatgt |
| 113161 | gatgatgctg gtaaaaacaa atctgtgcta ccagtcgtat aaaagcctag cacatatgat |
| 113221 | tatgtacagt atataatact taataataaa tggctattat tagcttatta tgtatttatt |
| 113281 | atgctatact ttttttcttt cttttctttt ttgagatgga gtcttgctct gtcgcccagg |
| 113341 | ctggagtgca atggcacgat cttggctcac tgcagcctcc gcctcccggg ttcaaatgat |
| 113401 | tcttctgcct cagcctcccg agtagctggg actacaggtg catgccccac accaggctaa |
| 113461 | tttttgtatt tttagtagag acggggtttc accatgttgg ccaggctgct ctcaaacccc |
| 113521 | tgacctcgtg atccacccac ctcagcctcc caaagtgctg ggattgcagg catgagccac |
| 113581 | cgcacctggc cttttattat gctatacttt tattgttatt ttagagtata ctcttactta |
| 113641 | tatataaaaa aaagttaact gtaaaacagc ctcaggcagg aacttcagga ggtattccag |
| 113701 | aagaaggcat tgttatccta ggaggcggca gctacctgca ggttattgtc ctcttctagt |
| 113761 | cacctccaca gtatagaggt ggaagacagt gttattgatg gtcctgacct tgtgaaggtc |
| 113821 | taggctgata agtgtttgtg tttcagtttt taaccaaaaa gtttatacgt gtttgtgttt |
| 113881 | cagtatttaa ccaaaaagtt taaaYggtaa aaaaattaaa aattttaaaa atagaataat |
| 113941 | agaataaaga tataaagaaa gaaaatattt ttgtacagct gtgcaatttt tttaaatttt |
| 114001 | taaatttttg tgggtacata ggtgtatata tttatggggt acatgagatg ttttgataca |
| 114061 | ggcacacaat atgaaataag cacatcatga agaatggggt atccatttcc tcaagcattt |
| 114121 | atctattaag ttgaaaacaa tttgactaca ctcttaagtt attttaaaat tgtacagtgt |
| 114181 | gtttttgttt taagccaagt gttattacaa tggagtcaag aaggttaaaa taaattttaa |
| 114241 | agtttatgaa gtaaaaactt acactctaag gttaatttat tattgatgaa aggatttttt |
| 114301 | tttttggtaa atttagtgta gcctgagtgt gcagtattta taaagtctac agtagtgtac |
| 114361 | agtaatgtcc taggccttca cagtcactca ccactcactc tctgacttac ctagagcaac |
| 114421 | ttccagtcct gcaagctcya ttcatggtaa gggacctaca aaggtgaacc atttttcatc |
| 114481 | ttttatacca tatttttact ctgccttttc tatgtttctt ctttctttct tcctttcttc |
| 114541 | cttYctttct ttctttcttt ctttctttct ttctttcttt ctttctttct ttcttttttt |
| 114601 | tttttttttg acagggtctt actctgtgcc taggctggag tgcagtgatg tgatctcggc |
| 114661 | tcactgcagc cttgacctcc tgggctcaag tgatcctccc acctcagcct ccctagtagc |
| 114721 | tgggactgca ggtgcacatc actatgcctt gcctggctaa tttttttgta gagaggaggt |
| 114781 | ctcactatgt tgcccaggct gttttctata tttagttgtg tttagataca caaatagtta |
| 114841 | ctattgtgtc ccaactgcct atagtattca gtacaggaac atgccRtaca ggtttgtagc |
| 114901 | ctaggagcaa taggttctac catctagcct aggtgtatag taggctatac catctaggtt |
| 114961 | tgtgtaagta caccctgtgc tgttcgcaca atgaggaaat cacctcatga tgcatttctc |
| 115021 | agaacgtatc cctttttcta gaaatggaat ctcataatat gtggtctttt gtgtctcttt |
| 115081 | catttagcat aatgttttag tttttttttt aattaaaaca tttgtgtggg tacataggtg |
| 115141 | tatatattta tggggtatgt gacatgtttt gatgcagaca tgcaatacat aataatcaca |
| 115201 | tcatggtaaa tggggtatcc atcccctcaa gcatttatcc tttgagttac aaacaatcca |
| 115261 | Rttacattct ttaagttatt taaaatatac aattaagtta ttgactatag tcaccctatt |
| 115321 | gtgctatcaa gtagtaggtc ttagtcattc tttctaacta gttttttgta cccattagca |
| 115381 | atccccgcct cgcccctact ctctccctgc cactacccct cacagcctct ggtaaccttc |
| 115441 | cttctactct ctgtgtccat gagttcaatc attttgattt ttagatccca caaatgagaa |
| 115501 | catgcgatgt ttgtctttct gtgactggct tatttcactt aacataatga tctccagttc |
| 115561 | catccatgtt gttgcaaatg attggatctc attatttttt tttaatggct gaatagtacc |
| 115621 | ccactgtgta tacataccac attttcttta ttcatctgtc aatggacaca taggttgcct |
| 115681 | ccaaatctca gctattgtaa acagtgctgc agcaaacata ggagtgcaca tatttctttg |
| 115741 | atacactgat ttcctttctt ttaggtatat acccagtagt gggattgctg ggtcatatgg |
| 115801 | tagctctaat tgtagttttc tgaggaactt ccaaactgtt ctccatagtg gttgtactaa |
| 115861 | ttcccaccaa cagttagcat aatgttttaa aggctcattc atgttgtatg taccagaatt |
| 115921 | tcattccttt ttatggctga ataattccat tatatcagta catcccattt tatttatcca |
| 115981 | ttcatcagtt gataggcata tggtgttctt ccacttttgg agtattaaga ataatgctga |
| 116041 | tttgaatatt tgtgtaccag tttttatgtg aacatatatt tttatttctt ttgaatacat |
| 116101 | acctaggagt ggaattgcta gatcaaactg tactgggagc tgaaagcatt caaattcaat |
| 116161 | ctcagtaagg tctaacattt caagaacagc tgggtcttgc agttatgtaa catgtaaagt |
| 116221 | ggaatgattg cctgatttca gttgatacag gtaaatcaac aatgaaggaa gaaagattcc |
| 116281 | tggataagtt tctcaaaatg ccactaggaa ttcttaaatc tcaccaaaag ctttcccaag |
| 116341 | atgtctgtct tatctctgtg tggtgtgatc aatggtatta attttcttct ttttggtcag |
| 116401 | ttttctatta cttttctatt tgaaatttct atttgaaatc tgccaaattt gttcaaagtt |
| 116461 | ttgaacttcg tcttctaaat ttttggaaaa attttgcaga ttttggtaaa aatgaagcag |
| 116521 | aaattaattt atgtgatttg catctttcat gagcattatg ttattgcttc ttttgcccta |
| 116581 | ttgtaattta catattttta aaaaagctat tcatgtgttg cttttggtcc catgattcat |
| 116641 | aattgctttg aggaataaat atgtttgtgg ttatacagaa gagattgctg aatgaaaaaa |
| 116701 | ttgtgattca tttgtatata aatgtaaaag ctactctagc tgagtaatta atggtttatt |
| 116761 | taaacaatgg cacttgacag gttctgtggc caagaacttc ttcaccaaat caaagctttc |
| 116821 | cattccagaa ctctcctata tatggtaagt acaattaatg ccatatgaca ttcataccat |
| 116881 | catccataac tcatccttta gttcctacct ttgcattttt taaaggctta tgttttgaat |
| 116941 | gatgttttta acagactaca ggaRtgaata caactcagat ttttgttgct gtgaaaatta |
| 117001 | aatgttatat gaaaagtaca atgaacaatg tctggcacgt attaagcact atgagtgtta |
| 117061 | actattattt taaagtaatt tcctaattat tatccccatc tcaatccctt ttcagtctag |
| 117121 | gcagttgtgg gcacatgaga cttaccatcc tgtagatttc attttcagac ttttctgcta |
| 117181 | ggtgtgatac aagtaaccag aggagcttga caaactattt tttatatttt ataaggctga |
| 117241 | gtgaacagaa ggttgaggaa tattttggag ttaggttaga tttgactatt ctagctgcct |
| 117301 | ctgagctttg caaatttggc tggtgtttat cagttgggcc ctaattcaga cttgcagctg |
| 117361 | tggtatccac tgcaccttta tgcactctgg ttccaaaagg gctatgctgg gatggtccag |
| 117421 | atggctctat ttgcattcat gaaaatgctt ggtgctgctg taaactctcg gctgtgtgtg |
| 117481 | ggctagttct tccccaaaac tgggcaggag atcgcactgc tataaatata taattaaaaa |
| 117541 | cattataatg tttatataaa gctttttgca tgaggattta cagaattatg ataggattta |
| 117601 | aaattgcttg tcatgttttg tgcatcttat tggaaaacta ctccaataat actcatgcat |
| 117661 | gtcaatgtat ctgggattct gagctggatt taggttcttc aacaatatat actctggcat |
| 117721 | aatttttttg tttttacaag ccttatatta gttttgtttt tgttttgttt tttgtttgtt |
| 117781 | tgtttgtttt tttggagatg agggctcact ctgtcaccca ggctggagtg cagtgacatg |
| 117841 | attatagctc attgcagcct ctgactccta agctcaagcg atcctcctac ctcagcctcc |
| 117901 | cagctagctt ttcatttttt tctagagaca gggtcttgct atgttgacca gtctggtctt |
| 117961 | gaactccttg cctcaggcaa tcctcctgcc ttggtttctg aaaactctgg gattacaggc |
| 118021 | atgagccacc atgcccggcc cttatattag attttctggc tagggaagaa taaagaaatt |
| 118081 | atcattttga aaagaatacc atttgttttg gcagaattcc cagtctaaac attgttggca |
| 118141 | tgagacctcc tatctttttg ttgagacacg cctatgaaag ctgaatggta ttctttcgat |
| 118201 | ctggcgtatg aagcagtttc ctgacccctc actctgggga acagctattt cagggcccca |
| 118261 | gtgcattcga aattggagga ggaggatgta aatggtctaa aggaaagtta tggagcttac |
| 118321 | cttagaaata tattttcttt caaatagtaa tacctttcta gaataatttt gtatttgtct |
| 118381 | tatctctatg cttgtcctaa cacaagtaca cttgtccctg tctacctaag agatggttaa |
| 118441 | gcttatgggg tcttagatat aatactttgg caaataagtt tcttttgtgc tgagtaaata |
| 118501 | ggtctgtgcc ttttagtggg aaataatgac tgaagttaag cctgtttgag actccccctt |
| 118561 | tcttcctttt tgtcttatct ggttccagtc agctttagtt actagtttta tcctaaaaca |
| 118621 | taattcattc cattttttat tgagcttact tgtgttttcc caggagggag ttctcaagca |
| 118681 | aagaacagtt tggtacaaaa atttacgtga cttgaaaagt tcacggtttt cctttaaagc |
| 118741 | tgaaaaacaa agagcttatt tctgtgctcc aggtaaactg cataatggct aggacaacta |
| 118801 | taaaatggct ggggccaccc ttcctctgac tgttttgaac accatgtcca gggcttttca |
| 118861 | ccccatgcag cctgcattta ctgaatgact gtggacttct gggtatcatt tttatttctg |
| 118921 | tgccatagtt tcttgtccta tgtcctcagt gttgtaagac acaccaacta gggaacctga |
| 118981 | agaatttaaa tttttattta taaaatgcta ctgatgctga tcccatcaat cggcattttt |
| 119041 | cttcctcgga gtggccccca gagatttcaa atggatttgg ctaggagggt ttgggtatct |
| 119101 | ggtagggcca tcaccacaga gtctgggagt tttattggca cgtttgggcc aagtaacttg |
| 119161 | ttctgtaagc cccatggtag gtactcatga catttgtttc atttagggag cttagtgatg |
| 119221 | ctgactgtga tggagccctg accctgcctg agttctgtgc tgcgtttcat ctcattgtgg |
| 119281 | ctcggaagaa cggctaccca ttgcctgagg gcctccctcc aactctgcag ccagaatacc |
| 119341 | tgcaggcagg taaggcctca ccatcaaYtg taaaccttaa gtacttttcc tccaggctgt |
| 119401 | gtaaaaaact tttgtttcct gactttttaa tgattgccat tctaactggt gtgagatgat |
| 119461 | atctcatagt ggttttgatt tgcatttctc tgatggccag tgatgatgag catttcttca |
| 119521 | tgtgtttttt ggctgcataa atgtcttctt ttgagaagtg tctgttcatg tccttcgccc |
| 119581 | actttttgat ggggttgttt gtttttttct tgtaaatttg tttgagttca ttgtagattc |
| 119641 | tggatattag ccctttgtca gatgagtagg ttgcgaaaat tttctcccat gttgtaggtt |
| 119701 | gcctgttcac tctgatggta gtttcttttg ctgtgcagaa gctctttagt ttaattagat |
| 119761 | cccatttgtc aattttggct tttgttgcca ttgcttttgg tgttttggac atgaagtcct |
| 119821 | tgcccacgcc tatgtcctga atggtaatgc ctaggttttc ttctagggtt tttatggttt |
| 119881 | taggtctaac gtttaaatct ttaatccatc ttgaattgat ttttgtataa ggtgtaagga |
| 119941 | agggatccag tttcagcttt ctacatatgg ctagccagtt ttcccagcac catttattaa |
| 120001 | atagggaatc ctttccccat tgcttgtttt tctcaggttt gtcaaactat gcagccataa |
| 120061 | aaaatgatga gttcatgtcc tttgtaggga catggatgaa attggaaacc atcattctca |
| 120121 | gtaaactatc gcaagaacaa aaaaccaaac accgcatatt ctcactcata ggtgggaatt |
| 120181 | gaacaatgag atcacttgga cacaggaagg ggaatatcac actctgggga ctgtggtggg |
| 120241 | gtcgggggag gggggaggga tagcattggg agatatacct aatgctagat gacacgttag |
| 120301 | tgggtgcagc gcaccagcat ggcacatgta tacatatgta actaacctgc acaatgtgca |
| 120361 | catgtaccct aaaacttaga gtataataaa aaaataaaaa ataaaaaaac aaacaaacaa |
| 120421 | aaaaaaaaaa caaaaaaact tttgtttctt aaaatgatgt ccagatactt tggggagtcc |
| 120481 | ttggtattta cagttattgt tctttaactg ctattagact ttcacatctc ataatgtgag |
| 120541 | gaatagggct gatctgggta agtgtgtatt acctgttttt ctcattctat accctctgga |
| 120601 | tggatttgaa ccaaagtcac ttaccctttc atgacagttg tgaaataggc aagtactgaa |
| 120661 | cctcccagct ggtataaaat gaagaggaag atcctgtaga gccaagtgtt aacttggcat |
| 120721 | ttttaggcac acccgagtgt ctccgggtgg ccctgggcaa tggagtggaa tggaactctt |
| 120781 | atacctaatt taaaagtcaa aaaattagcc gggcgcagtg gctcatgcct gtaatcccag |
| 120841 | cactttggga ggccgaggcg ggcggatcat gaggtcagga gatcgagacc atcctggcta |
| 120901 | acacggtgaa accccatctc tactaaaaaa tacaaagaaa aaaattagcc gggtgtggtg |
| 120961 | gcgggtgcct gtagtcccag ctactttgga ggctgaggca ggagaatggc atgaacccgg |
| 121021 | gaggcggagc ttgcagtgag ccgagatcgc gccactgcac tccagcctgg gagaaagagt |
| 121081 | gagactctgt ctaaaaaaaa aaagtcaaaa aattacatgc agtgaaatgt gcaaagtgtt |
| 121141 | gtcaccttaa atgtatgctt gatgaatttt tgtatatgta tacacccatg taactacaat |
| 121201 | cagatgaaga tatagaatat tctagcatat cagaaggtta gaatagaact gttatatttc |
| 121261 | tccttaataa aacagtctcg ctcaaattat tgttttggac cctttagatg ggaaagagtc |
| 121321 | atggtgagta taacatatgt attaattcct ttgggagagt agaaaaatta atagtgtgtc |
| 121381 | ttgccctttt caaaggcaaa tgggcactgg tctgatttcc attattcatg gaagtatagt |
| 121441 | gctcattgag aggccccaga aacagctggt gctttcaaca acctttctca ctttttaggg |
| 121501 | aagggctgtt aaagaaatga gagcttagtc atgggtgatt attagcaaga gagagcagtg |
| 121561 | tctaaagaaa ctttaattac ttattagtag agtatgaatt ttgtgattag atctgcaaaa |
| 121621 | ggccttagat tatcttgtct agccccttcc tcatgatgac accagattca aagtggcaaa |
| 121681 | tgtcttatct aagtttgtct cggggcctct gactctctta aataaaaagt ctgtcatttt |
| 121741 | ctgtgttaaa tagtgagagg agaggaactc aaccaaaggg cagaggccaa gtttgtggct |
| 121801 | gctgctaagt gacaggtaaa catgttgggg gaagagtgac ccattctggg atcttcttca |
| 121861 | aatgtcctca taaaatatgt gaaggggaga gttcagagag caaaacctac taatttctgg |
| 121921 | acttggattt tgctaacaat acctgctttc tgcctattgc aggctgtaac tctagcctgc |
| 121981 | tgcccttcct tagatgcttg gaagactctc atgggtccca ggaattagct tgcaattgag |
| 122041 | gagcatggga aactcctctt ttctctttgg tgtctttctg tctccatttc attctctccc |
| 122101 | tcgtcttgtc ttgtcttgtc tttttctttt cttttctttt ctttctgttt cttctgaatc |
| 122161 | agggcaaaga agcactggca ttctgggggc tatcttgacg cctacatggc tctcattgcc |
| 122221 | aaggcagtgc ggggtcatag aggagcccct tttcttggga ttggagacac atggggcttt |
| 122281 | tgtctctctc taaagggaat taatagctgg caaaRggcca agttctgttc tagggttata |
| 122341 | tttaatacct tcgccgtgtc ctctttcaag ccctggagag agtcctgggt aacctcatct |
| 122401 | cccataccca cactgagttt tctgtttggg ggtcatgact agtactgttt tattgttttg |
| 122461 | atgccatatg gccatggaac ctgtgggaca tgccatgact ccttatttga taagtcactt |
| 122521 | ttgtaggtgt ccaagtgttt cacagactat ttcaaaagtc actgacataa cggctgggtg |
| 122581 | tggtggctca cacctgtaat ctcagcactt tgggagtcca aggcaggtgg atcactggag |
| 122641 | gtcaggagtt tgagaccagc ctggccaaca tggtgaaacc ccatgcacta aaaatacaaa |
| 122701 | aattaaacag atgtggtggc gcacacctgt agtcctagct actagggagg ccaaggcagg |
| 122761 | agaatcactt gaacctggga ggcagaggtt gcagtgagcc aagattgtgc cactgcactc |
| 122821 | cagcctgggt gacagagcta gactccttct caaaaaaaaa aaaaaaaaaa gtcactgaca |
| 122881 | taatttttag acatctgaaa gccctgagct aaaagtatgt taacaggaaa gaagtaatct |
| 122941 | cttgattcct ttcctactgc caatggcttc ttttgacctg cttattcagt agaggggaat |
| 123001 | ctataaaaaa gagtgccaat gagccccatg tttcttcctg gacttttatg gagactgtca |
| 123061 | gcatgcattt atacaggaac aagggaagaa tccatcatat acacttcaat taaacaaaaa |
| 123121 | ttgcacatca tttctagggc ttgtgggatt aatcttaaat aataattata tagttctaac |
| 123181 | agaaaacatg aaagaaccca taaaactcta tcaaactcag tgacggtata ttttaaaatg |
| 123241 | ctttatcacc tatgtttatc attgtatgaa gcttaagaaa atggccagaa ccacctaggt |
| 123301 | ctcctctgtg ctttttttgc ttttctttgc aaaatatgag tgatcctatt catttttttg |
| 123361 | ttcattttaa tggcatctca tttagtgttt ttcttttttc tttctttctt tctttttttt |
| 123421 | tttttttgag acggagtctt gctctgttgc ccaggctgga gtgcagtggt gcaatctcgg |
| 123481 | ctcactgcaa gctccacctc ctgggttcat gccattctcc tgcctcagct tccaaagtag |
| 123541 | ctgggactac aggcacatgc caccatgccc ggctaatttt ttgtattttt agtagagatg |
| 123601 | gggcttcacc atgttagcca agatggtctt gatctcctga cctcatgatc cacccgcctc |
| 123661 | ggcctccgaa agtgctggac aacacgcccg gcctagtgtt tttttttttt tttttaaagc |
| 123721 | ttaactctta cctttttcca acttttttct aattgatcta tattcagtct ttaaaaaatc |
| 123781 | taatccagtt tttgataaga ttggtgaaga gtataaattt ggttgatatc ttcatgcatt |
| 123841 | cataactaga caagcttgtt ttctaaaata ataatttttt ttttgagatg gagtctcgct |
| 123901 | ctgtcaccca gacttgagtg cagtggcatg atcttggctc actgcaatct ctgcctcccg |
| 123961 | ggttcaagca attctcctgc ctcagcctct cgagtagctg gaactacagg tgcctgctac |
| 124021 | catgcctgac agatttttgt attttagtag agacggggtt tcaccatgtt gaccaggctg |
| 124081 | gtcctgaatt tctggcctca agtgatccac ccacctcggc ctcccaaagt gctgggatta |
| 124141 | caggcatgaa ccacccacca cacctggcct gttttctaaa ataatggaac aggaattgta |
| 124201 | tctgatgcca ttttacttgc aattatggaa atagtccctt ttccctaata gtaaaacttc |
| 124261 | aaggacagag ttgtattagt tggtggctta cccgctagcc ttttgatagt ttcatcaaaa |
| 124321 | gtctcctttt gatagtttca tcaagagtct ccttttgata gtttcatcaa gagtctcctt |
| 124381 | ttgatagttt catcaaaagt ctccttttga tagtttcagc tgggtggggt ggctcacgcc |
| 124441 | tgtaatccca gcactctggg aagctgaggc aggaggatca attgaggtca ggattttgag |
| 124501 | accagcctgg gcagcatggt gaaaccctgt ctccaccaaa aaatacaaaa attagccagg |
| 124561 | tgcggtggcg tgcacctgtg gtccccagct actcgggtgg ctgaggcagg agaattgctt |
| 124621 | gaacctggga ggcagaggtt gcagtgagcc gagattgcgt cactgcactc cagcctgggt |
| 124681 | gacagagtga gattccattt caaaaataaa ataaaataaa aataaaaaaa taaaaaaagt |
| 124741 | caccttttga taggtgattg ggtgggttct ggtagaaatt tttgccatag agtttgttaa |
| 124801 | aaaataccta tggagtactt actgcgtgag aggtaccaca ttgttcactg tggtcagaat |
| 124861 | gctgtatgac agagctccta ctcccatgaa cttgtgggca gttgatacgt gccatgaagg |
| 124921 | ggaagtacag gtgcagcaag agggtgcatg atcttaggga agatttcctt aaacgtttgg |
| 124981 | gctgagatgt gaaggctgag ctaaggacta cataggtgaa gaagagagaa gcttaggttc |
| 125041 | tgggaagggg gaacagcagg tatgctgggg aggctctgag agggggcttg gagcatagga |
| 125101 | ggagtgggaa tttcaggcca cagtggctgg cgtgcacaga accggggtag agaggctggg |
| 125161 | ttgaagctga ggaagaaggt ggggaacata tgacgggcaa taatgtcacc ttattttagg |
| 125221 | cagataagtg acacaatcag atttgctatg tgaaatgatc atgcaggtta ctgtccagag |
| 125281 | aacaggtttt gaggggcacg ggtagaaacc agaagactaa gttgtggtag ctggccagtc |
| 125341 | aggagcagcg gggtgcttgc aatggggagg tgacagtgat atggagagat gtggacacct |
| 125401 | tggagagaga tttagcaggt aattgacaaa attatagttt tggaaagtgt tagctttctt |
| 125461 | cacaggaaga tccgctcatg ataaagcatc agtcatataa taccagtcag agccttgatt |
| 125521 | gatagactag aaagagaaat cggtgaaatt cagaccagag taagcaaatg aacaaattaa |
| 125581 | ggtgcttgta ctcaactcca tgtggtgctg atactcagac cagtacttcc ttacttactt |
| 125641 | actgtatgta tgtatgcatg tatgtattta tttaaattag atagacacgt aggtaggtag |
| 125701 | gtaggtagat acaaagacag atgaaacagg gtctcactat gttgcccagg ctggtctcaa |
| 125761 | actcctgggc tcgagcaatc ctcctgcctt ggcctcccaa agtgctgtca ttacaggtgt |
| 125821 | gagccaccat gcctggccaa tggccagtac tttactgcag aaggatattg gcatttcagt |
| 125881 | ccctgatggg agtgggaggg tggggaagga ttaaaaacaa tgccaatcca cttttcacat |
| 125941 | ttgattcaaa tttttttaaa ccttgcatta caattttatt tcctaatagc ttgttgtgac |
| 126001 | aaatttgatt tgtagcttct gtttattatc tgccacagtc tgcttttcct ctttggaata |
| 126061 | ttgacaaatt ttctagctgt tgagatattt ctttcctaat tactttgtga caaagtagtg |
| 126121 | aagcagaatc cgcttctaag acatagcctg gtgtgaagta attagcttac caagtagcct |
| 126181 | gatctttcct cgtgtgaatt cactcaacta ccgaaataac agccagtgtc actattgcat |
| 126241 | attgaaagaa acccttaatg tgattctttt tttctttgtt gtaaatcagc aagaatttat |
| 126301 | gctcttattt ttatcattcc tgccaaccaa ggagataaat atttcgtaaa atcatgttgg |
| 126361 | gtgcacatca ttataagatc ttgcctattt ctaacaatag ttaaatgatg tatttcctta |
| 126421 | gttaatgtcc ctatacatta gaaactacac tagtaacata gttaagactt acatattgtg |
| 126481 | atagtgatgg ttattgtgga aaattactat gctataacat gaagtgttca gageacatac |
| 126541 | ccttcagcca gactgctgag agttcaagtc cctgctcttc catttatcag ctgtatgacc |
| 126601 | ttgagtaagt tgccctgttc tcagtttcat cacatgtgaa atggggataa tagaagtact |
| 126661 | gggttgtcca tataaagaac tgaaagtact acctcagcat gatgaatgcc atatgtggtc |
| 126721 | tatcagagct agctgatttt gttgttatat ttaacaagca cagaaaaRct gatgggaaac |
| 126781 | actgattact atggctatag gaaatattct gatatattct ttttgtttct tcttagcttt |
| 126841 | tcctaagccc aaatgggact gtcaattatt tgattcttat tctgagtcac tgccggcaaa |
| 126901 | tcaacaacct cgtgacttga atcggatgga ggtaaaagat cttcatatgc taataaataa |
| 126961 | ggatgtgtat ggagctaaat ccattggcct atgttcttta caaattctgg aaaatgaact |
| 127021 | ttttttttgt ttgaaaatga gcttttataa catttccttt tcatacttgg taattattaa |
| 127081 | tactttgatg ctcagccttt cagatctaga caccagatgt atctataatt tttttccctt |
| 127141 | ttctccctaa aactggatgt ggtaaagtaa gtaacataat ctctagacct tctagatgtg |
| 127201 | aaatgtagat atagccaaac taagacagaa aattctagcc aagttattta ctttaaccaa |
| 127261 | gctttaattc cctagtaaac aattttgatt Statttttat cagttgggca ttaataagct |
| 127321 | atactgaatc agaactgttt tgagaagagg tttgagaact ttatWtgagg gccccaaatc |
| 127381 | acattcggaa ctccctcagg gtttttagct tttgaaccaa atgaggactg atttggatga |
| 127441 | tccccagaag gaacttggag aattccccca gtgttgggcc atggatggaa gggcttagcc |
| 127501 | aagtgcttag gaagccttag ctatggatca ataaatagga gttgttatag ttattgttgt |
| 127561 | catcaccatt gtcattatca tcatcatcac cattatgatt actgtgatga caatttagga |
| 127621 | tacctgcttt tcccttcctt aaaagatctg gaaatgttta ataaatggag ctttattcac |
| 127681 | agactccata atagtcagag actattagag tcctctgacc ctagttgaaa ccactgcaag |
| 127741 | atgatagaag tcaaagtcag ggaacaggga catttcttta atttcttgtg actgtgcatg |
| 127801 | atgatttatt ctctcacatg aggccctcat gcctgtagca tgtgttatgg atggcaccgt |
| 127861 | ctgctcccct gtgagccagt caagcctgcc tttccagtgc ccctctgact aacatctctc |
| 127921 | tgccattttg cttctctttc tttgagttgt gtcctccatg gtactttcct cagatttcta |
| 127981 | ctcatgggaa aaagacataa agaaaagtca cttcaggagg aatgacagat gtgcatttga |
| 128041 | agaattttgg ttaaaatttt ttttttctgc agaatgctta tgacaaagag agagataaaa |
| 128101 | tagtttgcca cttttcatgg ccctcagttt actttggggc ttctagcagt gtttaaagag |
| 128161 | ctttgtttat caagccagga tggatacatc tatggctctc tgactatgtg tgtgtgtgtg |
| 128221 | tgtgatgggc cccctgcttg cataaaatga tgaagttatt agttggtctt ctttaaaacg |
| 128281 | gccataaaat ctgttacaca tgcacacgtc tgttttactg ttacacacac acacacacac |
| 128341 | acacacacac agtctctctc gcttgctctt tctctctctc taatgtcctg gatggcgtta |
| 128401 | catatattca cctttgtgga ttttatatgc agctagtttt atatactttt gcatacttta |
| 128461 | agtggcctcc ttggggaggg gagattaaaa actacctagt cttttttttt tttttgccct |
| 128521 | atccaaagaa gaaagcaaaa caacaacaaa aaatcccctc ttttccaacc aaattgttat |
| 128581 | ttttgaggat gctatggttt tagacaatga atgcagtaaa cagaattcaa ggacaaagta |
| 128641 | atagggttta ttgatggttt gttcatttaa gaagtacggg tcagattgca ataatgattc |
| 128701 | aatatttcat gttcttctaa actattactg attgttcttt ctactagtct tttaatctat |
| 128761 | catgaatgca tggagtttaa ttagattttg gaaccagccc tgtgatcgat gactccagta |
| 128821 | taggtggttg tgttctgaac aaagctgcag ctgatttagg tctcaSgtca tgagaaattg |
| 128881 | gcttagtgat acatcttttt ctaaagacag cagagctgtt agccatgtac ttatttccat |
| 128941 | taagaattac agatgataat ttcaagtata tttcttttta tgagtgtctc ttacattact |
| 129001 | ggatttcttt ctttttctaa acagctttat tgatagagac tttacatacc atacccattt |
| 129061 | aaagtataca attcagtaat ttttattata ttaacagttg ttccaccatc tctacagttt |
| 129121 | aattttagaa gaacatcatc agcccaaaga gaaacccaat actcatgagc agtcacttcc |
| 129181 | tattccactg tctctgccag cctctgataa tcactagtct attttctgtc tctagatttg |
| 129241 | cctattctgg acatttcata taaatggaag catatagtat gtagtgtttt gtgaccgtat |
| 129301 | tctttcactt agtataatgt tttcaaggtt cactcagatc ataacatgta tcagtgcttc |
| 129361 | attccttttt aatgctgaat aatattgcat tgtatggata tgctacattt tgtttgtcca |
| 129421 | ttcatcagtt gatggacatt tgcattgctt ccacttttgg ctattgtaaa taatgctgct |
| 129481 | atgaacatag atgtacaagt ttttgtgtgg gtgtgtgttt tcggttctct cgagtgtata |
| 129541 | cctaggagtg gagttgttgg attgtatcat aacttaactt tctgaggaac caccaaactg |
| 129601 | ctttccaaag caactatact attttacatt cttccactag cagtgtatga gggttccaat |
| 129661 | tttcccacat ccttgttaac acttattgtc tgcctttttg attttagaca ccctagtgga |
| 129721 | atgaagtggt atctcattgt ggttttgatt tgagtttcct aatgattaat gacgttgagc |
| 129781 | atctttttgt gtgcttatta gccatttgta tatcttcttt cgccaagtgt ctcgtccttt |
| 129841 | gtccatttta aaattgcatt acttgtctat tgaattgtaa gtgttcttgt gcttttggca |
| 129901 | tcatatctaa gaaaccattg cctaatatta aatcacgaag atgtacttcc atgttttctt |
| 129961 | ctaagagttt tgtttgtttg tttttttgtt ttgttttgtt ttttgagacg gaatctcgct |
| 130021 | ctgtcaccag gccagagtgc agtggcgtga tctcagctca ttgcaacctc tgcctcccgg |
| 130081 | gttcaagcaa ttctcctgcc tcagcctcct gagtagctgg gactacaggt gcgtgccacc |
| 130141 | aagcccagat aatttttcta tttttagtgg agacggggtt tcaccatgtt ggccaggatg |
| 130201 | gtcttgatct cttgacattg tgatccaccc acctcggcct cccatagtgc tgggattaca |
| 130261 | ggcgtgaacc actgagcctg gcctcttctg agagttttat agttttagac ctttacattt |
| 130321 | aggtgtgtga ttttgagtta atttttgtgt atgttctgag gaagaggtcc aactttattt |
| 130381 | ttttgtatgt ggatttctag tcgtcccagc accatttatt gaaaagatgt ttttttcccc |
| 130441 | attgaattgt cttgcaccct tgttggaaat caactgacca tagatgtaag ggtttatttc |
| 130501 | tgggttttca gttctattct attgatctat atgtttatga ttatgccagt accacagtgt |
| 130561 | cttgataatt ttagatttgt agtaagtttt gaaatcaaga agtgtgaatc ctttaacttt |
| 130621 | tttcttcttt ttcaagaata ttttgctatt ctgacttcct tgcatttcca tatgaaattt |
| 130681 | aggacaagct tgccaaattc tgacaaagat tgcattgaat ttgtagatca gtttggagag |
| 130741 | tatggccacc ttaccaatat taagtcttct aactcatgaa catgagatgt ctttccattt |
| 130801 | atttagatat ttaatttctt tcaacaattt tttgagattt ctttttatct acattgccta |
| 130861 | cttctaaatt atgttattct ccaaaggggc ctgatttata atgcaggtgg gaaaagacaa |
| 130921 | tgaaagaaat gcactgttat gatacattgt ctgttctatt ggagatagta gactttgaat |
| 130981 | agaaaattat atctggatac ttcgttttaa tgagtaaatc ccagcactat gtgaggccga |
| 131041 | ggtgggcaga tcacgaggtc aggagattga gaccatcctg gctaacacgg tgaaaccctg |
| 131101 | tctttgttaa aaatacaaaa aattagccgg gcgtggtggc atatgcctgt agtcccagct |
| 131161 | actcaggagg ctgaggcagg agaatcactt gaacctggga ggtggaggtt gcagtgagcc |
| 131221 | aagattgtgc cactgcactc cagcctgggc aacagagcag gactccatct caaaaaaaag |
| 131281 | aattacctga gtgaagactg ttaaatctta gaccagtttt ctactgagta atacttcagt |
| 131341 | ctctttcttc agaacagttt aaaaacaaaa gtattgctta tctctctggg gttgtttagg |
| 131401 | ttctgagagg taaaccaatt aactttttgg aacaccagat ggctccaggc atttactctc |
| 131461 | cttccttcaa gataaatgtt acaatttatc tttatttaat ttgtgactat gttttaaata |
| 131521 | taaacaatat caaaagacat atgttgacaa gtcttcctcc catctaccaa ctcacctttc |
| 131581 | aattcttaca tatctttcca gagtttgtgt atataaataa agcaaatgtg aatatatatt |
| 131641 | attcttgaac ttaatcttat aggcattaga gtgcatattc ttttccagct ttgaggtata |
| 131701 | attgacaaaa aatatatatt tatgatgttt tcatctatgt atatattgtg aaatgattac |
| 131761 | tgcaatcaag ctagttaaca tacctgtcac ctctcacata gttacctttt tttggtgtgg |
| 131821 | tgagaacatt taacatcaac tcccttagca gttttcaagt ataccataca ttgttattaa |
| 131881 | ctatagttag aatgtgtgtt aaattgagaa tttacaagaa gtaaatttac agtaagtaga |
| 131941 | ttttaaacgg tttaaaacta attcatactt gatagatttg ggtattcata ttagtgtgag |
| 132001 | gacttccccc ctgtttgcat taggagaaaa ttttaatctg ctcatcagta aaattctttt |
| 132061 | atcccacaac aagaaggctg gtttaatcag gtttagacac catctgcatg acagatcttg |
| 132121 | gcaaacaccc acattgtggg gccacttgtc agaacttctt tgtgtttgtg aaaaaataca |
| 132181 | taaagaacag gtttgttctt ggccaaatgg accagagttg ctatgcaatt ctgtggctga |
| 132241 | atcccagtct gaattcagat ttaatacatg tgccttaatc tgaatcattt ttttcctctg |
| 132301 | tgtatgtaaa atttcaatac aaattaacat atacctcact aggaacctaa aaaaaataag |
| 132361 | gtagtattaa atgcagggtt tactgcagat ggaaatggtg gcaagaaata atagctttta |
| 132421 | agacattttc ggctgggcgc ggtggctcac gcctataatc ttagcacttt gggaggctga |
| 132481 | ggcgggtgga tcacgaggtc aggagatgga gaccatcctg gctaacacag tgaaagccct |
| 132541 | tctctactaa aaaatacaaa aaattagcca ggcgtggtgg cgggtgcctg tagtcccagc |
| 132601 | tactcgggag gctgaggcag gagaatggtg tgaacccggg aggtggagct tgcagtgagc |
| 132661 | cgagatcgcg ccactgcact ccagcctggg cgacagtgcg agactctgtc tcaaaaaaaa |
| 132721 | aaaatttttt ttcatcatat gtgcgcatca cttaatttct gttccaacca gtttaattca |
| 132781 | tttacttttc tttttcaaag aagacatctg ttaaagacat ggctgacctt cctgtcccta |
| 132841 | accaggatgt aactagtgat gacaaacaag gtaggagaag aatgagtttt cttctttaac |
| 132901 | cagcgttcta cctgttacct tatctactcc tcttggaatg gtggcttcta gagcatatga |
| 132961 | gggtggttct cagcatgtaa acacatttgg atagactaag tagttcaaat cattgtcata |
| 133021 | aaacaatagt tcacaaagtg gtagcaagag cttgagtgtc cagagtcttc caactcattg |
| 133081 | gccccttagt cattgctgac cctcagccta ctcttaacac cttacactgg aatctgatcg |
| 133141 | tagcaattag gaaatgacag gaagctgaac Raattgagaa catgttgtgt ttgcggatgt |
| 133201 | gatcccagac atcagtgtaa atgcacattt gcatacagct ctaatccatg aggaagttct |
| 133261 | cttctggaca cagcttctgt ccttagtgtt tatctgtttc ttggctttgt gagtgtggga |
| 133321 | gtgtgacatg gagcccagct acaattttcc tatcttactg ggcagctgag aagcagtgca |
| 133381 | gcacagcatt ccgtgttaga gctactccca gggaagagtt ggcttgggaa tatctagaag |
| 133441 | tgtgggaggg accctctccc ccacctccta tactttgtat agtgctttat aatctaccag |
| 133501 | gtccttgctc atatactgtc ttattcagtc ctcacaaaac tgaatggagg ctggtaagtt |
| 133561 | ttatcacctg aaatttagag atgagtaaac tgaaattcag acactatgaa tcatttgcct |
| 133621 | agttattaag aaaaagattt gggacaagaa ccaagatttt ttctaagcct caagccagta |
| 133681 | ttctgcctgt tatttgatat tgatttacta cccatgaatg agaggcttat ttgaagggac |
| 133741 | taaaaatcag tgaagctatt tcaggtttta ttgttaaggc ctgccattga ttaggcattg |
| 133801 | tatgggggct taacatgaaa aataagtttt ccattaagtg acagtaattg catgttctct |
| 133861 | atttaaatac ttgcttggga gagacaggta gaactggaca ttgtctgcag tagcgtgtac |
| 133921 | tctgaccatt ctcctaactg cacagtggaa taactgggca aactgtaaaa atattgaagc |
| 133981 | ctggatttca ctcccaggga ttctcatgtc atcagtctgg gtgtcaggaa tgaaagaggg |
| 134041 | agaaactccc aatacttcct ggtaatactt ctcctatctg cttgaggtgc tgtctctgga |
| 134101 | aagttgaaaa tacatctttg ctctttgtag aaggagaacc tatttccata ctccaggagg |
| 134161 | tctttattgt tttagtaaat gctatagcaa cttttagagg tttgtgagac ctgtttttgt |
| 134221 | aagtgatgac tagcgattct ttgtttgaat ccaatttgcc tcagaaataa cattttatag |
| 134281 | ttttaatttt ttttcctctt ttctctttct ttttgcttaa aaaacaaaac acaactagct |
| 134341 | ttgaaaagta ctatcaatga agccttacca aaggacgtgt ctgaggatcc aggtaggaga |
| 134401 | aaaccatttt tgtatacttt atatataatt attttaaaat aaaaaaacag caagtcagtg |
| 134461 | agtagaagat tgatacctct gtgatagtgt gtctgtttct ctctatttat ttgtcttgta |
| 134521 | ttttattaca aggaaacctt ttggcttttt accctttctc tcttttcagg tatgagaacg |
| 134581 | gggtgatatc ttttcaagtt tttaagtttc actgaaagga tcacgaagga tcactccgtg |
| 134641 | tgaatatagg gtcttgttac taagtgcaaa attgagaagg cttgaggttg agatgatttc |
| 134701 | aacaagccat tttgggtttt gtgtagagta aggaacttat agggtagatc aattggaggc |
| 134761 | ccagagttgg tattttccat ttccaaagca gaaatctcaa atatttttat tatgcttcac |
| 134821 | caccaggaag gcaactatat gtgcttatat atggagtgga ttccaaggag aaaaataaaa |
| 134881 | gttgtccagt tttttggaga gtcagacctg tgtctgtgga gtcatggaga atggctgccc |
| 134941 | ctgtgtccgc agctctctgt tgatggagcc atcattgaac caagttgaat ctagttaaaa |
| 135001 | acaaagtggc tacagaagac attactattt ggagctctag ttataaggac atgaaaaaag |
| 135061 | ttgttgcttt ctcttttgct gttttatttc ttgtatattt ttaataagtc aatcatgttt |
| 135121 | gttgtgataa gctagctatc atcctaggct tatttgttga acattggaac agaggaatca |
| 135181 | atataatgtt gaggcttatg ctgtggtaca atattttcca aatgtcaagt tcattcagag |
| 135241 | tttcaaagag agatgggtgg agggagagga aggagttggg ggagtgggag aaaaatatgg |
| 135301 | tagggggaag gagagagaga gagaacatga gaatgagaat aaatatgaat gaatgtggat |
| 135361 | gatgaatttc aattatggaa gcctgagaag gtgtattttt aggcagctcc ccagttggtt |
| 135421 | ctggtacata gcctattttg ggaaccacca gctctggaag gtgatgctac aaaatagaaa |
| 135481 | attaaggtta tagatcaacc atgactccaa aagtgatggg aattcccaag cttaattttg |
| 135541 | actcttgaaa cagatttgaa ttcattcctt attgggtgaa atgctctgtt aatgtaggcc |
| 135601 | aacgcctggg gtcagttctt tctagctctt atttctggag ctaaagtgga tgcattccag |
| 135661 | tttttgtaaa cttcatgttg tagtattagt aaaagctgtt taaatttgga gtgttaaatt |
| 135721 | ggtgcttctc caacagcctt tgaggatcat aacttttcct actttttttt ttttttttaa |
| 135781 | gggattttag cacaagatca agtgtcctct ggggcatgag ctttagatct catttttaac |
| 135841 | attccttcat tgactaggac atggcatcac caaataaaac ttgtcaggtg tgccacagca |
| 135901 | tgagaagtat gtcggaattg atttctcgtt gtgcctggca gcatgctatg tgttttctga |
| 135961 | tttgttcttc ccactactgc ccctctctgg gtatgaggga gttcctcatc ttaaacatag |
| 136021 | ttaatccctc cttttctggg aaatgtttgg attcccagtg ggagacacaa gcgtttcaag |
| 136081 | gtggtgattt agaattctgg atggctgtcc ttgccatgtg gacctcagtt cagttgctgc |
| 136141 | tttttggagg gttaatctct aggcctgctc ctctttccat tcccatttcc tggaaagatg |
| 136201 | acatgccttg agtgttctag gaaggtggca gtccttttcc ctctttccag ctacacgaga |
| 136261 | agctatgatc cacttgttgt aattgctgag agtgtctatg ttagtggagc cttaagagat |
| 136321 | tttttaaacc aatatcccat tccaacctaa ttcagcagag caccatgcac aatgRtggtc |
| 136381 | tttttcttga tgtttctggc ccatgttgag gtgttttcag ctggttcacc ctcacagtgc |
| 136441 | tgtctttaga ctgttgttgg tgattctagc agctgctcca ttgccaacta tcaccccatg |
| 136501 | tttttattgg agtagagcag ttctgtcctt Scattacctg ctcctacctg cacctccttt |
| 136561 | gctgtcctag aagaaactaa atttagaaac cagatttcca ggcttaaaaa attaggagcc |
| 136621 | tttatttatg cactctacaa tagtaatatt ttgcaattct aaagtacgtt cagtttttaa |
| 136681 | attactttca ccttatctgt ccctgtgaga aagaaactgc atttttattg cctttgtttt |
| 136741 | ctaggtgagg agactgaggt tcagggaagt aacagctagt tagtgtcatg gccaggagga |
| 136801 | gggccttagt ctcaggactc ctggcctggt ctctagtcct gtctctccac tactcatcca |
| 136861 | caccctcacc ccatctgtct gtctgtctat ctctYaccag cagggctcac ccctccttcc |
| 136921 | acacccatcc ctaccagcaa aaagacaaag ataatggcac ttgacaaagg aattaacagt |
| 136981 | caccatggtc tgatttgggg aacagtattt tatcatggcg gggacctctc ctacccatgt |
| 137041 | atttcttgaa agcagtgggg cctgagggag aagagggcca cggagccagc tgcaggcagc |
| 137101 | ctgcgtagag ggcttcccca gctctgtggt gagtctggtc cactctgtcc acccagcccc |
| 137161 | aaggcagcct cctgagctgg gagagaggct tcctgaggtc gctttctttg ctgcctgggg |
| 137221 | tttgtaggag ggagggaggt ctttttctgt ctatatccag gtgtgggtgc acgataatgg |
| 137281 | gcaaaagggg actcaaactc cttgggcttt ccaggctttc ctgcactctg gcagcgtttc |
| 137341 | agctgtttga agttggaaga tctctgtgtt ctcactacaa caagccaaaa tgcttgggtt |
| 137401 | gggagaagtg tatctcttgt gcagctgacc agtagtttgc tttgtgcctt gttagcattt |
| 137461 | atgaggattt tcacctcccc tctctgtcct gttatctaac ccaaggattt taatgatcat |
| 137521 | cataaattca ggctgcttca taccaaggag ggtttttatc tttggagcaa tgtaaaagag |
| 137581 | gatgagataa tgcagtgaga cattttcttt ttggaagaat tcatgcagac aaagaaatta |
| 137641 | cattcttctt gttctgtgtt actagcacct tttctatgat tttttatgac attttatgat |
| 137701 | gaaaaatttg caatgtttgt aacacctgag catagctact ggttcctaaa gttgtcttcc |
| 137761 | atagaggatt gaaagcataa aaagaggatc agtgtttttg taaatgtttc taattactcc |
| 137821 | tcacaacttg tgatggggaa gaaggctgta tgggcgttgg tagtgagcaa gttagtttat |
| 137881 | gatttaaatt tgatgaggaa aagtcatatg gctgggaaaa gattcagcga tattgttcat |
| 137941 | aattctctgt atgatttggt cacagacatg tgggacacaa ggatattttc ttacaggaga |
| 138001 | agctgtatag ctcatttcca cttacttgtg agagctttgt ttttttgtgg ccatgctgct |
| 138061 | ctggtagtgt attttttttt ttttaaaacg tgtagttaga tagacttctt cctatccttc |
| 138121 | agggtagact agttcaaaat ctattactag actatcatga tagaacaaga aaccctgaag |
| 138181 | tgtgaatcca tcattatata aaaagacaac cccctttcaa gggggttgtc tttttatact |
| 138241 | ggaatgtctt cattaattaa cttggatttg agaatggcct ctcaaatagt gcttgaccaa |
| 138301 | aggaaaaata tgctggtttt tctttccttt taatcgtttc tttagatttt gaaataaaac |
| 138361 | aaggaaatca tgaaaatgtg ggagccgtcc agttctccac atgggcactt ttacctcaca |
| 138421 | tttacctKtt gatgtgttct gtactagccc aggtgctgca tataaacttg tttaactgca |
| 138481 | gaaatgaaac ccaagcaata tctttgtttc agcaactccc aaggattcca acagtctcaa |
| 138541 | agcaagacca agatccaggt agtgttcgtt taatttctgc tttaatgccc tttgtgccgt |
| 138601 | gcccctgcct agaaataaga accacaaacc aaaacccaac ccatgatttc ctcttccttt |
| 138661 | agatgtaaag cttaggatat gagccatttc gctcgtgcat ggcctgatgg gaacatgcag |
| 138721 | taatgctggg agccttggtt tctgtggtca ttagtgctag aaggtgacca tgaatgccct |
| 138781 | acggcccctc aacgtgaagt ggattgtgtg agacaaccca gttgttgcaa gatgataact |
| 138841 | tataggggga agtagctttg gaaagaggaa aatatgggta aggtaacaga cacgaaaatt |
| 138901 | tgctatcatg acaaggtgaa caccaaaaag taaaaagtct gggtaataaa atctacagta |
| 138961 | gggaaagaaa atgcttctga taacttcagt gaagcttgca gtttcaggta agcgatgttt |
| 139021 | atggtctgtt tgagcctatc ttagtgcccc tStaagcttc acatttttta agaggctagt |
| 139081 | acaagagaga aagccagtaa ggcagagctg tggttgatgt gagactcaga agagaggtgc |
| 139141 | agaggggaaa tacagagaca ctaatagaat taatccatta aggaagggaa atggtattgg |
| 139201 | tactgtattt cactggtaga actgcagtgt aagataggat aacgaggata ccatRtgtgt |
| 139261 | gttttctgtt ttgctttggc tgttagtgac atttttccct cctttgccta ctagacacaa |
| 139321 | tcctgaaaaa gtaaatttaa gtctgatcat attttatatg cttgaaatac tgatatttaa |
| 139381 | ggaacctcat ctctttgtaa gtcaaagagt tatcctttag tttatatgtg cattttccaa |
| 139441 | attggggttt tgtttccttg tttatgtaag atacagatat ctatattttt atatttatgt |
| 139501 | gtatttaaaa acaatagagt aattcattta ggtaggaaga aaaggtgtag gcattctttc |
| 139561 | tgtggtcaac acatttactc atcctttgta aaaggatgaa tggatctttt acaaactagt |
| 139621 | ctgtactagt tgtgggatat gctttcatag taggaattgc ctttgaattg cagaaatctg |
| 139681 | ttatgtgtac tatgttaacc ataaatcgaa cctcaaagtt ttagcttctg ctgacttcct |
| 139741 | ctctccctga aagccagtga aattataaaa tctcatagcc acacgttctg ttattaatgt |
| 139801 | gtctggctaa attttacata ttatcctttc agagattgcg cttttggtct aatttggagg |
| 139861 | attttatttt cattacttct cactgggaag aagagaattg aattgttttt atcctccttt |
| 139921 | ttgaaataat aaagttttgt agaaatataa gggaaagagg gaaaatcacc attttaacac |
| 139981 | cactgcattc atcattttgt taactccctg cacatttatg ttttaattgg ttgcagcagt |
| 140041 | catggttata caatctcact tattttatat tcagtaactt tccatatgaa gatataaaca |
| 140101 | tacaggttat atatgaaaat aaaatgaaaa tgaggaatgt gcgctgtggg attgtgtgct |
| 140161 | taacagtgtg cacgcataca cattatttac acacacatta tttctttttt gagtgaaaga |
| 140221 | tgtcactaaa gctatatgtg gcatagaaag ctcttttcat ttctagctag ggtctctaat |
| 140281 | tgtctttttt aagtgaagtg ttcccattta agaaccttgt ccttccgtat cagttccctt |
| 140341 | tgattcttgt tgaaagagcc agagcattct tccctgctag tttctgcctg ttgctgcctt |
| 140401 | gcctgtcttg ccagttatgt tacagggacc accccagaac acgtcacatg ccattatggt |
| 140461 | ggctttatta aagttacaac cttgtaagac agacagaacc ctcaactacc acaaagaatc |
| 140521 | cttgagaaat ttattcagga aaataaaaac tcacaatgtc aaaaacagct ctgtcaagaa |
| 140581 | ctgaaggggt ttaatagtaa ctgatatatt Staacttcat aaatgccact ctaatagcta |
| 140641 | agtacgaaga aggttttcag aggttaattt gtctctctag gatgtacctc tgactcagag |
| 140701 | gtcgttttct gctttgacat gaagctagtt gagaagctaa ataaaacaga tgtatacatc |
| 140761 | tacatcttag ggccttttcg tgatcatcgt attgggaacc cgtccttaaa cctattccta |
| 140821 | agcagtgtat ggagttgaat tttatttgga aagtaagtag gtctccaata aatattctag |
| 140881 | ttgctatcac caaagtattc ccccgccaac tcataggcag tatgagaaca ttttaaaaat |
| 140941 | gggatataga aacagagatt ggagtttgaa tcgaactgtg taactttcta tctgaccttt |
| 141001 | tgcaagttat ttagttcctt tttaaaaaga tgatattacc agagttacaa tctcagaagt |
| 141061 | tgcctagtta attatgcctt gctatagatt gaggaagaag gttgaaaatg agacttttgc |
| 141121 | tttctcaggg aaagacaagc tagacccctc aatctgattg atgtgtgttt tcctttccca |
| 141181 | gcttcgtttg tggcatattc ccctgaacat tgaattctta ccctcagctt atctgtccca |
| 141241 | attccacatg agcctgtcat cacatttgca agtgaacagg agaaaagttc tctgatcagt |
| 141301 | ttataccatg aaataccatg aagcccttct gttagaacat ggaaatgcct aagctgaggg |
| 141361 | gctccatatt attcttatta acaattccaa tagaaatgga tttgaattta cacaaagttt |
| 141421 | tttttggggg gttgattcag atctaaggta taataagaac aaagaggttg aaaagatccc |
| 141481 | aattcatagt tggatttgga caattattgt acatagtggc attcaatggg taatagaatt |
| 141541 | tatggctgct ttggtagctc atcggttctt tgtagtcact ttgacttcgt gggtattttc |
| 141601 | catttcaccc caacttcaga gagtaaagct ttgctatttc gcttctgacc ttcccttatt |
| 141661 | ttgctacaga tcttactcta gcacctccat agaagaggcc atgaaaaggg gcgaggaccc |
| 141721 | tcccaccccg ccacctcggc cacagaaaac ccattccaga gcctcctcct tggatctgaa |
| 141781 | taaagtcttc cagcccagtg tgccaggtga agtgcccctt gccccctgag cccttccatt |
| 141841 | tcctgttgga gccagcggat gtgtgtctgc cacagtggcc tctgtgagtc cggagaaaga |
| 141901 | gcagacctgg cagtttcgca ttccgtgaag gtcttcacgt ggctatggta ttcttgggag |
| 141961 | agaaagcaat tctagacaca tatgtagcac atgtgtctgg agccttgccc acacttgtta |
| 142021 | tggatcaaga attaggaaat tctctaccaa gaggataaaa atgcttcact cccatgcagc |
| 142081 | ctgctgagtg tgtggctggg gtcttactct ctgcatcatt ggtaatttgt gaacaagata |
| 142141 | gatSgctgtc tgtcatttcc tcagatgcct gtgtggtctt ctatgaaaga tcttggagat |
| 142201 | taaggcctag ccagaacctt ctcctgcatt ttacacttat ggagaagatc ctgtgtcctg |
| 142261 | taggatttaa tgctgagggg actctgaatc tgactgatgg ttgggtggtt ggtggagggt |
| 142321 | cctagggcaa gggctccttc aggtgggctg ctctgggctt ctggggatgg gatgttcttg |
| 142381 | agcccgcagt tgtgtagtgg ggctggaggt gggggaggcc cagaggggcc ctgtctgctc |
| 142441 | ccttggcttc ctgtgaagta gaagataccc tcctaaggaa cctaaggaat agaagatggg |
| 142501 | aggcatgcct ttgctttagt gaaggtgata ggatacggtg ctgtcccaag tgttattttg |
| 142561 | ggagcagcaa gggcctttcc actgactcat gcaggcaagc tataaataca atgatttcct |
| 142621 | ggaacttttt ctccatctat gtgcatttta aatcaggcca gcctcagatg ctagtgattt |
| 142681 | gcagattatt atcttttctg ctaaagcaag aaaagacttt ccttggctca cacattagca |
| 142741 | tgacctctgt gtctgggcag agctcagcat gttttatttg ttctaataaa gtattctcct |
| 142801 | cacagtaaag ttatagattg ttttttaata tactaaaatc tggaactctc tatcaaattt |
| 142861 | aattctgtgc catgagttca tcagacaaaa atttgccttg aatttcctct catactttga |
| 142921 | tctgaattct ggcataaatt tcatagtgac tggtatgtag ggtgggagta accacaggtg |
| 142981 | atgatgctat aaaaatttca catggctggc ggattatgac ctggaagatt ttcctatgga |
| 143041 | tgcgagaaaa ttaaaacaat ctgggaagct atctcagtac ctttaccttg caagaaggga |
| 143101 | ggctggaaat gctcacaggt ctctctcagg ttgtgtgacc cagtaaggag atgccagctg |
| 143161 | tactggagaa ttcacatgct gtggttttta cgacactgca tcacatagtg gaagagacaa |
| 143221 | aactgggttt ggagctcagc tcagctgctt attcactgtt tgattttcag caagtagctt |
| 143281 | aatgtttatg catctcagat tcctaatctg tacagtagag ccaatactcc atggagttgt |
| 143341 | aaggttttgg tgagataata aacataatcc aaatatgggt tgaatattcc ttatccaaaa |
| 143401 | tacttggggt cacaagtgtt tcaggctttg gatatttttg gattttggaa tatttgcatt |
| 143461 | atatgcttac caattgagca tataataaaa aaatctgaaa tgcttcaatg accatttcct |
| 143521 | ttgagtgtca tgttggcact aaaaagttta gggttttata gcattttgga ttttggattt |
| 143581 | ttgggattag ggatactcaa cctgtagtac tgtatttggc ccacaggcct tagtaagcat |
| 143641 | gtaaggccca tattcccgtg gcctgcaagt atcaagaatt taatgcctga aatggcaagc |
| 143701 | ctccaccgag gaggataaca ccccgacttc cttgacccct agcagacaat tctgtgattc |
| 143761 | ctcctaccca gtttctcaga aggctcgcaa aaggactgaa ccccagttgc ctgccgtagt |
| 143821 | acaccattca ttgatgtact ttttcttgtt ttgcttccct cctgccctgt ctcactttcc |
| 143881 | cctcactcga ataatatacc tgttctcaag ttcatatgtc aggggcttct ttagagaaaa |
| 143941 | cctgaattaa gacagctttc tttgatatct ctgtgtcaat gtgacttcaa gatgaaaagt |
| 144001 | attttggttt gataatgccc tgacgatctc ccttgtggta gggcctgata aatctaagat |
| 144061 | ttagacaaat aatagctagg tgggtgatgt ctctgttcat agcctaattc cattgtcttt |
| 144121 | tcagtacaaa tttatacaga tgaaatttta atgagcagtt taaatttatc acatgaagaa |
| 144181 | tagaacattt ctgcagtatg aaaagcaggt ggtataattg catctacttt atttattttc |
| 144241 | atgttggtta attctcacag aaaagtataa gggtctgtct aaattatata aaattttagt |
| 144301 | gtattgcata ttgctagaaa ttatcgaaat gctgacaaac tagaaatgca tgaaatagag |
| 144361 | cttaaaccta ccagaagagt ctactcactg agaatcatgg tgtacattta ggcatccctt |
| 144421 | tgctgcactt atctttatgt atcactttag ataaatggga tcacactatg cttgctgttc |
| 144481 | tgtatcctga tctttccaat taaccatctt tctatttcat atagaggatt gcaccgttcc |
| 144541 | ctttaaggtg tgcatgatat ggatatacca tggttgactg tgctttgatg gagagatctc |
| 144601 | tcccttcttc tctctgcctt ttctcttccc catactttcc tcttaagctc acccaccacc |
| 144661 | ccacgacccc agcccccgtt cctttctcac ccttgcaaac aaacctctct ctctctccac |
| 144721 | cccgaccccY gtgtcctctt catacccctc tgtttctctt tttgtccttc tcccccatca |
| 144781 | ctccttcaca ggtatgagcc tcccctcact ctccccttgc caagctcccc cacaccacca |
| 144841 | ccttgttctt ataatataaa cgaggcagag gttattaata atttcatgtc cgtatctttg |
| 144901 | tttcatcatc taattatttt cttaggttaa atcctgctgg ttggagttgc agggtcaaag |
| 144961 | gctgtagatg tttaaagtgt agctatatgt tgccaaaggg tgaggtggaa cacctgtcaa |
| 145021 | cttgctccat catcaacact gtctgaaagt gtttcctcac aatcttgcca acactgcttc |
| 145081 | atgttcatct ttttaatcat tacctgtgta ataggcagtt tcatgtccct gattcctagt |
| 145141 | gaggctgaga atctttttgt attgttattg gccataaata ttccttctct tatgatttgc |
| 145201 | ctgctcagaa ggcttggcca gcttctgttg ctacatctct ctttgcctaa ccacggggac |
| 145261 | cacttctttc ttcttcattt tgcctacgtt ctggacctcc ctcctgtcag gcacttggga |
| 145321 | atgtgagggt ggccatgggg aagcctacct cacctgctgc tgtgctgttc cttgtccctc |
| 145381 | cagccccgcc ccccatatcc cctccttctg ccaactttat cacttaagac agactgcctg |
| 145441 | ggtcaccctg tttatgacaa tagaagaata aggaaaggta aagaaaagag gggaaaataa |
| 145501 | tgtgagatga agcaagatgt ggacttgagt tgaaagggaa agataaaatg aagcccttgt |
| 145561 | ttaataaaat gagactcagg attgtgaaaa gcagtgattg aagctaaggt gctgatacag |
| 145621 | agtggcttaa agtggagtga tagtgtggaa aggaaatagg atggagagaa gatgcaaaga |
| 145681 | aaaatgaaag gaaaatgaag ggaaagatgg gatggttgtt acataggagt gatgatgttt |
| 145741 | atccaaagga ataagcagga aatagaataa aaaattgtag aaccaatgga aggagagtcc |
| 145801 | acagactctt ggtgtaggat tcaagcagca tttaaaggaa tagcttagaa gtaggtggca |
| 145861 | ttaatgcctt tacatttatg aaaataggta acatgatatg gggaaaaaga acaaagggct |
| 145921 | gatttagaat gtaaatctat aaaaatgaat aaggccaact ttggaaatat agaacagttg |
| 145981 | gcttcaactt tggtgtctgt aaagagagtg tgaatgtgtg atttatagta gcctagatga |
| 146041 | ttccagggct ctctaggtac tggcatcaga cccgaggcat gtgttttcat aaggagaagc |
| 146101 | ggatacacta tgatcgctgt gtgtaacagg gagatcacca tttgtaatag ctgacccagt |
| 146161 | tcttggagcc attgcctcac agtcccacac agctgctgtg tttgagagat agcaaaaatg |
| 146221 | tgcaagaaag tctaagtcag tgacaatgca agtatgcaaa acctagaggc agtcagacct |
| 146281 | ggaatttgct cagtgatgtt tggattttat gtctctatat ttatagctat gaaacatctt |
| 146341 | ttattactga gattcctgta agaaaaatca cactaagctg gtacctaacc tccttttcct |
| 146401 | caatgaaaca gttattttca tgaggtaatt tttttttcat aaaaaggggt ttcttctgtc |
| 146461 | cacggagtca gtaagaacta cccctctctt ctactcttga ttcagacctg cctcttctca |
| 146521 | aagccatgcc tgaggtttgt agcctgaggt ttccatggtg acagtaaaaa gccttagctc |
| 146581 | tgccttgttc cagtcattga gcatctgcct ggcagaaggg gcagagatgg tgtggaggag |
| 146641 | acatagccat tcctttaaag tttcagaccg ggcattttca tcatttccac tcaaatctag |
| 146701 | ctgggggacc taggcccatg gatagaccta gttgccaggc agctgggaaa tgtagtcctt |
| 146761 | ggcagggcag ccatttccca gaaatacgtt tcttactgca aaaaaggaga aggggatgtt |
| 146821 | gatggtcagt tgtttttttt taaaaccaca tcccatatgg ccaaatacat tgagattcac |
| 146881 | acattaaaac cattgtgatg gctgaacaga acacatccag ggctagtgtc tgtccatggg |
| 146941 | tagctaattt cctatctctg ctttagatat actgataata acaactcatt ccatggttaa |
| 147001 | gagactatgg tacatccatc ctatggaatt gtatgaagct gttaaaaaga acaggcacgt |
| 147061 | atactacatg gagagctatc tcggataaat taagtgacaa aagtatgatg cagaatgtgt |
| 147121 | atgtttataa atgtgtagac aatttcagga tgatgctaaa gaaactggta atgatagttg |
| 147181 | cttctgggca aggggaacag tatgactgag gacaggactg ggaaacaagc ttactttctg |
| 147241 | ttgaatgcct atttgtatct ttttaatttt ggctcatgta cagatgtcac ctattataaa |
| 147301 | aaatttgctt ttcactcttt agctgcaatg atggtatctg aaagtaccag agatgtcttt |
| 147361 | cctgcctgag gttacctatc tcaatcttcc ctacgattct gtattcaggg tatctttatg |
| 147421 | accatctgct accacctcct ggaatgcagc tgaaatgaca gaaRtgttcc gagcactttt |
| 147481 | tttttttttt tttttttttg agatgaagtc tcgttctgtc acccaggctg gagtgcagtg |
| 147541 | gcgtgatctc agctcactgc aagctccgtc tcccgggttc atgcccattc tcctgcctcg |
| 147601 | gcctcccaaa tagctgggga ctacaggcac ctgccaccac gccctgctaa ttttttgtat |
| 147661 | ttttagtaga gacggggttt caccgtgtta gccaggatgg tctcaatctc ctgacctcat |
| 147721 | gatccacccg cctcagcctc ccaaagtgct gggattacag gcatgagcca ctgcgcccag |
| 147781 | ccccgagcac tcttttaagc tctgatgcgt tcttcagatt ccatttcagt gcctttccaa |
| 147841 | cactgaggag tagttctgga ggacctccca aagattacat tgtgagatgt atagttgggt |
| 147901 | aaaacgtcat ggtaagggct ctgtcttaac ttggaagtgg ctgtgtttat tgagtggcga |
| 147961 | attgagaagt ctttcctcgg ttttctataa atgaatattt gtttctgtaa cttaagaagt |
| 148021 | aggtaaactc cttgggagta agagatgttc ctaataccaa acttaccttt caggttctct |
| 148081 | aaaagatgaa tttggccaca aaagcagctc tgaccttata tatttcctta ctgtattttt |
| 148141 | cctgtgagtg ctacttgttt cttttaaaat ctggtaggac tatgggattc ctgacccacc |
| 148201 | ccaaaccttt gggcccacag tgcacagcta tccaagggtt caatttggca gtctgggcag |
| 148261 | tttttttgtt ttgttttgtt tttttgtttt ttttaaatga aagcgttatt gagatgtaac |
| 148321 | ccatatacta taaaattcac ctttttaaag tgcacaattc agtgatattt agtatattca |
| 148381 | tagtgttata tataactatc agcactatgt aatttcagaa tattttcatc ccccYcaaaa |
| 148441 | gaaaccccat acctattagc aatcattccc attcccccct tttcttcctc tatcccttgg |
| 148501 | caaccactaa cttactttct gtttctatgg atttgcccat tccggaattc tgtattaata |
| 148561 | gaatcatata atatgtggtc ttttgtgact ggcttcattc acttagtata atgttctcaa |
| 148621 | ggttcatcca tgttgtagca tgaatcaata cttcattcct tttatggctg aataatcttt |
| 148681 | cattgtatgg ataaaccaca ttttggatat ttgggttgtt tccacttttt gactattgtg |
| 148741 | aataactctg ctatagacat tcatgtccaa gtttttgtgt aaacatattt tttctttctc |
| 148801 | ttgagtatat aggtagggct ggaatttctg gttcatatgg taactaactc tatgttgaac |
| 148861 | attttgagga gctattgaag tattttccag agcagccaaa ccaatttatt tcattagcaa |
| 148921 | tttatgagga ttccaattgt gtttcttcac gtcctcaaaa cacttgttat tgccttttta |
| 148981 | attttagcca tcctagtggg catgaagtgg tattgtgatt ttgatttgca ttttctaatg |
| 149041 | gctaatgatg ttatacatct tttcatgtac ttattggtca tatttatgtt gtctttggag |
| 149101 | aatgttcaga atctttgctc atttaaaaat tgggttgtct ttttattgtt gatttgtaaa |
| 149161 | tgttctttat atattttgca taaaagttcc tttacagata tttgatttac aaatattttc |
| 149221 | cttcgttctg tgggttgtcc tttcatcttt ttgatggtgt cctttgaatc ataaaagatt |
| 149281 | ttaattttga tgaggtctaa tttatgtatt ttgttgcttg tgcttttgct gtcatatcta |
| 149341 | aaaaaccatt gcttaactca aggtcacaaa cactcctctt ttcttctaaa agtcttatag |
| 149401 | ttttaggtct tacatttacg tcttcgttct gttttgagtt aattttttta tatggcatga |
| 149461 | gttaggagtc caaattcatt tttctgcatg tggatataca gttgtcccag taccatttgt |
| 149521 | tgaaaagact attaattatc catttaattt tcttggcacc tttgttgaaa atcaattaac |
| 149581 | cataaacata aggatttatt tctggactct cagttctgtt ctgttggtct ataggtctat |
| 149641 | ccttatgcca gtaccacagt gtcttgataa ttgtagattt atagtaagtt ttgaaattgc |
| 149701 | gaagcgtgag tcctcctgct ttattctttt tcaggattgt tttggttatt ctgagtccct |
| 149761 | tgcatttcca catgaaattt atgatcagct tgtcaatttc tgtaaaaaca aaaaagccag |
| 149821 | ctgggatttt ggtatggctt gcattgaatt tgtatatcag tttggttagt attgcggttt |
| 149881 | taataacatt aagttgtcca atgcatgaac acaggatgtt tttccattta tttagatctt |
| 149941 | taagtgtctt tcaacaatgt ttcagtatac aagtcttgca tttcttttgt taaatgtatt |
| 150001 | cctaaaagtt ttattctttt tgatatacat tatacattat ttcttggaga tatccatgtc |
| 150061 | agtactgata attttgtatt tcttttatat atcatctata tgctggctgc tctgttaaaa |
| 150121 | accgtattat gaaaaatttc aaatttatgc aaaagttgag aacctccacc cccatattat |
| 150181 | tttgaagcaa atcccaaata tcatataatt gtatatataa actctttgta tatctctcta |
| 150241 | agagataagg tcttaaaata cttgagcaca atacctttgt catacataac aaaattaaca |
| 150301 | ataattcttt aatatcattt aatatttggc cagtgttcct atttcctgaa ttgtctcaag |
| 150361 | tttgtctttt tatactagtt tgttccattc agaattcaat cgagggctac atgcacattg |
| 150421 | cacttgtttt ggcatatttt aagtctctta actgataggt tcttctctca gtcttttttt |
| 150481 | tcctctttgc ttttcattta ttcataaaaa gtcatctacc ttgtagactt tttcacattc |
| 150541 | tgtattttgc tgattgtttg ttcttccatc ccatgtattt cctataaact aataggtagg |
| 150601 | tctaggcttg attagagtta gtttagattt ttgacaagaa tactacctaa agatgctgtg |
| 150661 | tacttcttac tgcaccacat taagagatct ctagtgtcta atgcctttat ttttgtggtg |
| 150721 | tttgattgat taatgggttt acgtagtatt ggcctgatcc atgtgttatg ataaagatcc |
| 150781 | cagcgacctt tcaaataatg gttttatcag ctattgatca tcattgccta catccagtgg |
| 150841 | tgtgtcagtg aatgtttaac aaccggccac ttgccaattt ttatggtgta aacactcctg |
| 150901 | ccatggctaa tttcattcct actgatgtgt tactgaatgt ggagtttgga acagatgcac |
| 150961 | acagttggct ctcacaagct ggcatgagct ttttccagca aaccactgct tagatccatt |
| 151021 | atttcattgg tagttgcaaa atggcgatac tctatttttt ctacctttat taattgggac |
| 151081 | tatggtttat ttaagcattt tcctattgtt ggacatttaa tttgtgacta ttatgaagct |
| 151141 | tgtgagctga aggagatcat aaaacataat tctcagtaca tcagtaaatt ctttatatac |
| 151201 | atctgaattt atagaattgt atgaaaactg aaatagactt cagtaatggt gaccagaaaa |
| 151261 | gtgtcttatt caaccagtat ttaatgagca tttactgggt acttggcact ttggaggatc |
| 151321 | ccaagaggga tatggatatg cccccaaact ctgctaatca atgtcacagt tgtgtgtgat |
| 151381 | tgttagaagg gtttagagtt tgggtggttg ctaactagaa tgatcagtgg atttgtgggc |
| 151441 | agggggtgca cagggctggt gagctaaact ttgaaggata tgacttttca gtcaaatcct |
| 151501 | attctttttt gacatctgtc ttgatttact tcttatgact agtttcctgg gtgtctacta |
| 151561 | tagtagcctc aaatctggct tgcttgcttt gttctttcct tcataaagca acaggattaa |
| 151621 | ttttctgcag cacacctatg atcacttccc tactcaaaac tcttcaggac ctactatgta |
| 151681 | atcaccaaat ttctggtgga actgaacccg aagccatgtg attatattag ctcagtaagg |
| 151741 | gacacagtac tgtggcctgg agtgcctggg gaggagtgta ttacaggaag gaagtagaga |
| 151801 | ctagaagatt caagttggct ggctggccaa gaaaaatgga gactggtgga gaaggagttt |
| 151861 | gtagacggtg tctttgggaa gtcttaggag aacatcccat tagggtggta gggttaagcc |
| 151921 | agattgcaaa agattgtggc ataagcaagg aactttggtg tggagaagtt gtcatcacaa |
| 151981 | agtcattttg aaatgtcgaa ggtttagcag ttgttctgac tgtatgtcac attttaaaaa |
| 152041 | ataatggtag tggccaggtg cagtggctca tgcctgtaat ctcagcaccc tgggaggccg |
| 152101 | aggtgggtgg atcacctgag gtcaggggtt caaaaccagc ctggccaaca tggagaaacc |
| 152161 | ccatctctac taaaaataca aaaattagcc aggcgtggtg atgtgtgcct ctaatcccag |
| 152221 | ctattcggga ggcttaggct tgagcctggg aggtggaggt ttcagtgagc cgagatcgtg |
| 152281 | ccactgcact gcagcctggg caaggacaga gtgagactct gtcgatagat agatagatag |
| 152341 | atagatagat agatagatag atagatagat agatagataa agatacataa gatagataga |
| 152401 | tagaggtaga agagagccag ttccaagatg atagactgtt tttatagatt gccttcctcc |
| 152461 | taacacacat ttgtattctc tctctctccc ccaccgcatt attcagcgct aatgaaagtg |
| 152521 | tgatggaaac agctgctagt gaatgaatac taactaatcc tgaaaaaaag atgtgtactt |
| 152581 | cttgaaattt tagtttgatt tacctgaaaa catatacaga aacacaacta gtgatttcaa |
| 152641 | ccaaccctag agttttaata gtaaaatgct taggaggaat gcatttgtgt aaatgtaata |
| 152701 | tgagagagga aaccagacac ccatctttga ccaagattga atctcatgca tttttgtcac |
| 152761 | ttaagtctgc ccatgtctcc atccaggacc tagttaaccc ttattgcgta tcctatggta |
| 152821 | caggaaaaat gagttattag taaacatgga taaattttag aatcatgaag tgtcaatgct |
| 152881 | aaaagagaac ttgaatgtca tcttggccaa ctctcacata ttaccaacaa ggagactaag |
| 152941 | atccaggcaa tataatggct ttgttctcca tcagaatctt tctttccttt tttttttttt |
| 153001 | tttttaagat ggagtctctc tctgttgccc aggctggagt gcagtggcgt ggtctcggct |
| 153061 | cactgcaagc tctgcaggtt cacgctgttc tcctgcctca gcctcccgag tagctgggac |
| 153121 | tacaggtgcc caccaccacg cctggctaat tttttgtatt tttagtagag acggggtttc |
| 153181 | accatgttag ctaggatggt ctcaatctcc tgacctcgtg atccacctgc ctcggcctcc |
| 153241 | caaaatgctt ggattacagg tgtgagccac cacatcctgc ccagaatctt tcgaagttgg |
| 153301 | gaaatctttg tttagatctt tgttgcagca gatttggtat caaagaaagc tatctacatt |
| 153361 | aaattaattt ggagggcaca ccctactact tttctacggg caataacact aattaatatc |
| 153421 | taaagttaaa ggatgtatta aagaatgaat atggtaacct ttgcaaggca ctcttcaatg |
| 153481 | tgtttttttc tgtgtttcct tctatgtgat ttaaaatgtt agtagatttc tctgaggtac |
| 153541 | ttcagggaga taggaaagga gcttttgccc teatagatac ttggaaataa tatatctatt |
| 153601 | ttcttttttt aatttcaaaa atgaaattat tattaaaagc tctaaatata gcaagaatat |
| 153661 | aagacaaata gtatactgaa catccagctt caacaattaa tcaactgtgt cccatcttgt |
| 153721 | ttcatatctg tcttccccca cattattaat tatacttttt tctaccctta gcttaaaaaa |
| 153781 | aactttattg aggtatatag atcataaagt ttatccattt taagtgtaaa atttaatgat |
| 153841 | ttctagtaaa tttatcaaat agtgcaactt tcaccataat ccagttttag gtcattttca |
| 153901 | gcactccaat aatactgcac atacctgttt acagttaatc cccatgccca gccccaggca |
| 153961 | accactaacc tattttgtgt ttctgcagat tttccttttc tgaaYatttc atataaccga |
| 154021 | atatacaata tgaggtcttt tctgtctgac tttgtttact tagcatgatg tttttgaggt |
| 154081 | tcattcatgt tgtatcatgt atcagtactt aattcctttt tattgctgaa tagtattcca |
| 154141 | ttatatagct atatcacatt ttgtttatcc agtcttcagt ttgacagaca tttgagttgt |
| 154201 | ttctgctttt tgtctgttat gaataatgct gccattaata tttgaataca agtctttgtg |
| 154261 | tgaacatatg tttcatttct cttagctaga tatatagggg tggaattgct gggtatgtgg |
| 154321 | taaatttatg tttaactttt taagaaatgg acaaattgtt ttctaaagta tctgcaccat |
| 154381 | tttacattca catcagaaat gtatgagggt tactatttct ttatatcctc acccacactt |
| 154441 | attattatct ttttgattat cctacatcct agtgtgtgtg aagtgatatc tcactgtggt |
| 154501 | tttgatttgt attcccctga tgattaatga tgttgagcat cttttcatgt gcttattagt |
| 154561 | tattcctgta ttttatttga tgagatattg tgttagtctg ttttcacgct gctgataaag |
| 154621 | acatacccaa aactgggcaa tttacaaaag aaaggtttag ttggacttac agttccacgt |
| 154681 | ggccggggaa gcctcacaat catggcagaa ggctaaaggc atgtctcaca tggtggcaga |
| 154741 | caggagaaga gagcttgtac agggaaattc ccatttttaa aaccatcaga tcttgtgaga |
| 154801 | cccattcact atcacgagaa caccatggga aagacctgcc cccatggttc aatcatctcc |
| 154861 | cactgggtcc ttcccacaac acatgggaat tatgggagat acaagatgag atttgggtgg |
| 154921 | ggacacagag ccaaaccatg tcagatgtct actcaaatca tgctcattaa aaaattgggt |
| 154981 | gtttgtctta ttgttgagtt gtatattctg taattcttta tatattctgg atacaaatcc |
| 155041 | tttatcggat agatgatttg caaatatttt ccacagtctg tgacttgtct ttttgttttc |
| 155101 | ttaatgatat gttttgaagt acagaagttt tgagttttga caaagtgtaa tgttaaattt |
| 155161 | tttccttttg gattgtgctt ttgatgtcat atctgagaag tctgcctaac ccaaggtctt |
| 155221 | gaagattttt tcttgttgta aaatttttat agttttagct cttacgttaa gatcttgaat |
| 155281 | ccattttgat ttaatttttg tataaggtgt gaggtaagga tctaagttaa tctcttcgta |
| 155341 | tatgaatatc cagttatccc agcactattt gtcaaaaaat actctccttt tcctgttgcc |
| 155401 | ttggcacctt tgttgagaat cacttgtctg ttaaatataa gggcttattt ttggacactg |
| 155461 | cattctgttc tgttgattta tatgtttatt tttatgctag cactatactg aatgtttttt |
| 155521 | aacacagggg agtcatacat aatcttgcaa agaggtaatc ccacgactca gaagtcttta |
| 155581 | agtatttgct acaatggcat attttttaga tagtatatag tagcttattt acttcatttg |
| 155641 | tggtttgtag catgttcaga aggatccttt gagctccttg aggttgcagt acttctttgc |
| 155701 | tttgaagcct tctgtcatgt ttattccctg aatgtagaat accctctgtc tagctgttta |
| 155761 | tcctccatct ctgcattttt ctgctctacc tgccctgtag gttcaaaatg tggtactgat |
| 155821 | tactgctcga tagtagcctt gtgtccttgg caaggtcatt ttatttctcc aggtcttaat |
| 155881 | tttctcatct gtaaagtgaa agaatcagat tttatctcac tacttttcta aactctgtcg |
| 155941 | ttaatatttt ccttgttgaa cagttacaag gatgtgtaag ttaatctctt ttaactggtg |
| 156001 | aaaaagtata ttctgttcca tagagaatta aatgagcttg tatttcgggg ggtggtgtgt |
| 156061 | ggtcatcagg ggatagaggc aggtatttct ggaaaggtgt tcttggcaaa cattttgtta |
| 156121 | catacactgg tgtatatgac atacatgtcc atcactgtat gctgggtgat caagagatat |
| 156181 | gtataaggtc atacatgctt ttatgattag aaaggagaga aagaatgaac aatttctgtt |
| 156241 | acaaagaaat agccattttt atgaacttgc caagaagagg aatggtcctt actgatgaaa |
| 156301 | tccttaaagc tttgcatgtg gctctttact tgattgatat ttaaaagtat aggtcccact |
| 156361 | gggaggctcc tttcaacatt ttccattcct tgagtgtttt tccccccttt ggtattcgaa |
| 156421 | tgcttttatt ttcttcacat aaaatattcc tagttagggg ggatgggagc ccctttccac |
| 156481 | agctgaagtt attccaagtt ttgttccccc tgaagaatcg gctctctgtt cctgaagctt |
| 156541 | tcaatggcca catctgctgc tttatttcca ctcaataagt tggaatatat tttcatttag |
| 156601 | ttattgatta ggggctatgt aaaaatctga aggaaattag taaatagtaa tgtttatgaa |
| 156661 | taacatttta cctagaaatt tctaagcgta atgaaataag aggataaaaa ttgttagagg |
| 156721 | ttggagtttt ggatgaaggt accaggaagc agagatccaa taaatcagta tcagaatctg |
| 156781 | catgtctttc aataaactta agaaattgtt ttaaacacta ggaattatta gtctgtcttc |
| 156841 | tgaatttagg attaaatgac agtttctttt cctgaagttt ttaatgtggt tttcaggatt |
| 156901 | ggggtattta ttattttcct ttggattaca cttataaaaa tttttggctg gacaaagcct |
| 156961 | tgctgatacc tagtatccca ctttattaac tataacactt gacgaataaa cctgcaattg |
| 157021 | cttgacgaat atggtcactt tttaaagtat gtgggaacaa cattcttctc tcgtcttctt |
| 157081 | gccttctttt ccctcccagg aaagcagttg ttactattaa caagtatatt gtcatctttt |
| 157141 | gcaactgcac actagattat tcagctattt tttttctttt ttttttcctt tgccttccac |
| 157201 | catgcgatca aagtattttt taaatttcac tttgcatgaa ggtgtacttt ttcttttgac |
| 157261 | tgattagggc atactggata aaatatttgg atccaatttt aagtcaagag cagactgaaa |
| 157321 | atattgtaag agaagatttt taaaattaaa tttaattcca attttcaacc actgtgtgag |
| 157381 | tacctactaa acataaggca gtgtgtttag gtgctgtggg agacaaaaag atgaattaga |
| 157441 | cagaattgat agaatgtagc tagattaata tctcccacca ttaaaggaaa ggaacacaga |
| 157501 | attataaatg caaggaagag atttttcagc agcaaccgaa caaggaaaaa tgcatgagtt |
| 157561 | aatgctatat atatatatat atatatatat atatatataa tttttttttt ggaaagagtg |
| 157621 | gcaactgagg gaaacctgag gctttgggtg cacttctgac tctgcctgct tccttgcaga |
| 157681 | agctgtgtgt gtgagtaggc aaatcttgag agttctcatc gagaccctag gtctagagca |
| 157741 | aagtgaaatg aatgagtact cgtaattttc ctttttcttg actaagtgga aatgacttca |
| 157801 | ggaaagagag ttggaaaagt aaataatcat agctgatatc ctgatattgt agaggggaag |
| 157861 | gcccagagct tcatgctgaa ctggggacaa actgaagtct gagaagttcc tccaaggcca |
| 157921 | ttgatagaac tccaagactt aggagaatgc actctgctgc agggttttaa aatgttgatt |
| 157981 | attttggtat ttccttcgta attctaaata gcatatttgt gtaacttctt gtagacttca |
| 158041 | atttttgcca tcatcttccc tcccccaKta tctttctcct ccacccttcc aatatagtta |
| 158101 | tattataatt ttggttagat taatatttag tgtttatatt attattgtta tagccatgta |
| 158161 | agacatatag taaattactt ttttttttta cttgcacagg tttttgtttg ccttggagtg |
| 158221 | aataatcaac ttgtttcttt tcaaagtact tgcccttgat tcaaccacag acgtcttcaa |
| 158281 | ttttccagag ttcctcttat gaaattctca cacctcaggt gttctgtcag tttcagcttt |
| 158341 | ttgaagcaat agaaactgat tgtcttcaat gcctggtaca tagttttcat catgggatct |
| 158401 | ctctacactg tcatccaagg attcccttcc tctttttccc atgttggatt tcttttatta |
| 158461 | ctgtatcctg tgtctttctc tttctttgtt tactttcttg ttttggtgaa gcacatcctt |
| 158521 | cagcagcttc cttagaaagg gtgcatggag ctcaatttga gaccctgaat gtctaaaaat |
| 158581 | gtatttaatt atatcatcat gcaggctgct agtcttggat atacaatcat cagttggaaa |
| 158641 | taagttttct ttagaatttt gatggaattg tgccaatgga ttctagcttc taatgatata |
| 158701 | tgaagaagtc taaaaccatt tggacatctg atcctttgta tatgacttgc tctcttctcc |
| 158761 | tatgccctgc tgaaaacttg taaattattt tgactctagt actttgaaat atcatgatat |
| 158821 | accttgtcat gagtctgttt tcttccattg tcctggatat tcagtgagtc ctttaaattt |
| 158881 | agaaattcat attcttcatt tctgggaaat tttattgaac catttaattg attatttttc |
| 158941 | ctgctgtttt ctctgtttct ctttgtggat cttttatttg gctactaccc tattttctgg |
| 159001 | aagatttcct cagattattt ttgacctttc tattctcttt ctgctatctt ttaatttcca |
| 159061 | agtattcttt ttgttttgtt ttgttttctg aatgttcttt gtaaatagca tcccattatt |
| 159121 | tcatcttagc aaatagcatt tcttatgtct tactgagact ttctctggtg actctagtta |
| 159181 | aaatttcatc tgtcactctc taccccattg tctcttttct cagtagaata tgagcttcat |
| 159241 | aagggtatag cttttttttt ccctgttttt cccatggata ttttcccaat accttgaaca |
| 159301 | atacctccca catagttagt gtttaaatgt ttctggaata aacttaatga tcattctgat |
| 159361 | gatattaata atatatattt taacgttttc tcctagtaaa ggtctagtct gtttctttga |
| 159421 | ggtcatttct tcctgttttt attgatccct accttaggtt agaacatttt ctgcagatat |
| 159481 | ctggtaatac ttagtcatcc acttatattt aataatgaag gataaaaagt tgcttggaat |
| 159541 | atatgagcaa attgatggaa gttgccagct attggcttca tggtagaatg gtctggccag |
| 159601 | gccatcctgt tggagatccc ttgatatcaa ctctttagat gtctcttcct ggctgatctg |
| 159661 | attctctgga acacattctc ctaatctcta gaaggaagca gcagtctggg agcagaaggg |
| 159721 | aagaaggctg gaagaatctc agcatccaaa gtgcatattt ttactgaatc tgtctgtttt |
| 159781 | cagcatggcc ctcatgcctt catctgggcc tggtgccttc cctgaaaaga aaactatatc |
| 159841 | tagcattttg ctggagtgag aaacatgctg aaactgtgca gaatgtggga gaaaaaactg |
| 159901 | ctcctaaact gcttcaatca gtcttttttt ttttttttaa tgaggatgga acataatcct |
| 159961 | atagatttta agatagttta aagaactatt tgttgatcac ttactgtatt ttgagttatc |
| 160021 | aggtgagaca gatataaggc atagctacta cagcagaggg gactgtaaga gtagggcccc |
| 160081 | tgaagccaaa aacagcatga agaatagcta gaaccagatc ctgaagggtc ttgtcaaagt |
| 160141 | atgccctttt cctgaagggt aggaaataaa tccactatta gttcattgct ttaggttttt |
| 160201 | attgctttcc tcattttacc cctacattca gaggtgtttg atgccaccaa ttcctgagaa |
| 160261 | ctggggggct tctgtggtac aaattKgtta ctccctgctt tctcagatct gtctagtcag |
| 160321 | ctattagttg ctcatctgtt ttcccagttc caaatttaaa caagagcatt taaacagttc |
| 160381 | tcttcatttt tgtgttaatg agttaagagt gagattagtt acatgtattt gaactgctgt |
| 160441 | ctatactcaa aagtctttgg ataggacatt taataaaacg ttgggtcata cacaagaaac |
| 160501 | aaggaatttt acacagtatc aagccaagat tttagtttaa agtggtccca ggttgatagt |
| 160561 | accctcagat ttctggcaga agcgatagag ggaagaggaa ttcactttcc acatgcctca |
| 160621 | aagttttcct gtcggtaaag ttccaaggaa tttgagttta gagtcacagg ttgctaagca |
| 160681 | cactaggaaa tgaagcacaa tgagcaagaa caaacaggaa atacaaagcc ctcagatact |
| 160741 | gaaattatat aaatatgttg aaagaaaaga ggaagaggcc gggcgcggtg gctcacgcct |
| 160801 | gtaatcccag cactttggga ggccgaggcg ggcggatcac gaggtcagga gatcgagacc |
| 160861 | atcccggcta aaacggtgaa accccgtctc tactaaaaat acaaaaaaat tagccgggcg |
| 160921 | tagtggcggg cgcctgtcgt cccagctact tgggaggctg aggcaggaga atggcgtgaa |
| 160981 | cccgggaggc ggagcttgca gtgagccgag atcccgccac tgcactccag cctgggcgac |
| 161041 | agagcgagac tccgtctcaa aaaaaaaaaa aaaaaaaaaa agaaaagaaa agaggaagaa |
| 161101 | aatgaatggc caagaaatta taaaaatgaa aaaaacattt tcaaaaaaga ccaaatagaa |
| 161161 | cttctagaaa agaacaattc tgtaacataa actcgaagac tggattttgt tagacattaa |
| 161221 | gaacgactaa agagggaatt ggttacttgg acagtagatc gtagaaaatt gtctagaaca |
| 161281 | cagtacagtt agaaagctct tgaaggagag gggagagaaa aatggtagaa atacaacatt |
| 161341 | taaagaatca ctgagaattg accaaaatta atgggttgct acatggttca gaaattccag |
| 161401 | tgaagctcaa gcaagtgata aaatgatggt agagaaatac atgtgcagac ataggataaa |
| 161461 | gtttgatatt tcagagtacc caaaacaaag aggagatctt aaaagcaacc agagagaaga |
| 161521 | tgggccatct aacaaaagag tgatttgact gagagctggc ttcttaatag caatagtaga |
| 161581 | aaccagaagg cagtggaatg ttaccgtcaa tgtgctggga gaaaatagct gtcaatttag |
| 161641 | aattctatac ccaattaaat cctctttcaa gaggggaaaa atgacttttc aatcaaataa |
| 161701 | actaagtttg ctactaatag acttctcaat taaggaaatt ctaaaagaca tactttagac |
| 161761 | atcataaaat aaactgtaat ggaaattagg aagcttttag cactgagtga taatatcaca |
| 161821 | aataaaaacc tatgatattc tactaaagag gtacttggaa aaaatttata accctaaatg |
| 161881 | cttacattag aaaataaaag ctacaaatta gtgaactaag caaccaattt aagatagaaa |
| 161941 | aagaacaaga tatgcccaaa gaaagtaaat ggaagaagat aagcagcatt aataaaacag |
| 162001 | aaagcaaaca tacaatacag aggctcaaaa aagttaaaag ttggtttttt gaaaagagta |
| 162061 | acaaaactga caaatgccag ctgagattag tgaaggaaaa ctggagtatg aggaatggga |
| 162121 | aaagacaatt acacatgcag agattaaaat aataataaga ggggggtatg gacaccatta |
| 162181 | tgctaataaa tttgaaattt taggcaatga ataaatgcct agaaaaatat catttatcag |
| 162241 | aatcgactta agaattagaa agtctgaaaa gtcctataac aattcaagaa gcagaatcat |
| 162301 | agtttaaaat cttgtttaaa gaaaactccg ggctcaggtg gctctcctgg gaaaattttt |
| 162361 | atttgatatt caagaaaaaa tttccaatct tatacaacct atttaagagt ataaacaggg |
| 162421 | catggtaggg gtttgggggt ggtgctctcc acctgaatga agcataatag aggaccatat |
| 162481 | gagaaaggaa atcacttgca ggtctcactc atgaagatag ttgcaaaaat cctaaacaaa |
| 162541 | atatcagcta accaaatcta gcagatataa aaaacttacc aagttgagat tatcccaggt |
| 162601 | ctgcaagggt gttttaatat tagacaatcc agaatgtaat ttaccacatt aacaaattat |
| 162661 | aaggaaaaaa taataccata atctcaatgg ctagagttac agctctgatt aaaatccaac |
| 162721 | aacaaattat cctgaaaaga agtctcaaaa taaggatagg aattttcata ataatctatt |
| 162781 | tttttcaggg attatatatc tatagtttta ttaagacaaa aactgagagt gtggtgtgaa |
| 162841 | gtttacattc aaacaaagtt ttcaaagaaa tctaacacat gcctaaaaag attttacaat |
| 162901 | gtagctctag atccaagtct agataatatc aagaactgat ggatctcatg actcaagaca |
| 162961 | gagcattttg ggtatcagtt acttcttagg atttgttaaa aaatggtttt gtgtgtgtgt |
| 163021 | gtgtgtgtgt gttttaaagt gaaccactgc ccagtatgaa agtttaatct cctgagacca |
| 163081 | aggcttttga aatcactcaa ctcttgagtc aattcagtga aacttgtgct gtcagtgact |
| 163141 | gaactctgcc accaatggtt tcagagttca aagctcaaaa gagaatggct ccaaaagttg |
| 163201 | tcccaactaa gagcatgggc cctttacttc cctcctgtct tatctcctct accaccctct |
| 163261 | ttcccttccc caacacctca gccagtggct aggatgaaca agctgatatt tataacttta |
| 163321 | ttattggaaa agaaagggtc ttctaatttc aggaattagc acctctgagg cagaatgatc |
| 163381 | tgcttgtact gtatactctc caataaaaga ccttccttcc tcagtcaatt tccatctcca |
| 163441 | aaatggcaac tctggtaact tattaactgg cttagtcctt tgtgagacta agtacattaa |
| 163501 | gtttaggcag ggaacaccat ggcttataga tttcagacat tcacagcttt taaagtctct |
| 163561 | cacagtccgt atttatggtg ccagtgacat ttttttcaaa ggtaaaatat tctggacata |
| 163621 | gaagcaaatc attagttttc tgttctttgc attgcttcac catgtcccct atcaggcccc |
| 163681 | aaattttaat caaaatgatg ctacccatct ccctttccac cctccccatt cctccctctc |
| 163741 | ccacataata caccagtaat agccaaaaac tacacatKtg ctacgctgta aaaatgtaga |
| 163801 | gttaacacta ttgggaagaa agttgtgcgt tgcggagatg ctctttgatt atctacagta |
| 163861 | tttttttgct ctcccacaca cccagttctg caagttttgt ccttcataaa gggccctttg |
| 163921 | ctttttctca gcaaagtgca gaaaaggttg ccttgaagca cttatccccc ttcccctcca |
| 163981 | ctgggacggg tgtgcaaact atacagcttg gaatggcttt aaaacacttg ggctgctgca |
| 164041 | gggcacagaa acgatactgg ctcttcaaag ggcatcttct caagctacct ctatggtgtc |
| 164101 | aagacaagta gaacttcttt ccttcccacc tgaaacccac agtcagatgt gatagaggct |
| 164161 | ggtactcagg agagttaatt cttgtcatca tcctctttgg ggtaggaatg ccacgtcagc |
| 164221 | ctttcctcat agaaggatat gacaacccgt gggcatttga cattgacttc cttggcaggg |
| 164281 | acgaggtcag cctcatcaga atttttccct ttcatcagga acatgagctc tccactggag |
| 164341 | tctgtagctc caataatctg ctccggctcc aaaccctgag caaagcctcg tggcttttct |
| 164401 | gactcttctt tcttcttctt tggtttgctc tcctctccct tatcttcaga atcagaatca |
| 164461 | gctttgtgct tgcctccctc tgatttgtct atctcatgtg ctgttttctg tgacggcaga |
| 164521 | aactcatgag gtcagggcaa tccaagttct cttctgactc ccatgtgttg tcctcatctg |
| 164581 | agaatccctt ccactttagg aggtactcca ctttgccctt taccaatcga cggtagagaa |
| 164641 | ctttttccac cacatattcc tgttcctcct cttctagcac ctcctccact ttcttcttgt |
| 164701 | tttgtttttt ctccatagcg tgtgtgtaaa gggtgatgct gctcagagca gtgcccagga |
| 164761 | gccggaaagg aatcggtgct gcgctattgg cgcattgggt cggccagggc agtggcgctc |
| 164821 | agaaaagcag caagggtagc tgcaaaggag ccccttgctg aaatggtgta ccgcaggcct |
| 164881 | cggctaatgg ccctcctctc agccgaacaa aagagccctg cactctgtgc tgcccactgc |
| 164941 | ccactgcctg caccgccttc ataatctatt aaagggtatc tacaacatac ctacagcaaa |
| 165001 | caccaacacc ataccttatg gtgagctagt aaatgctccc ctttgagatc aagagaagac |
| 165061 | aagaatgccc attatcacca tttaaattca acttttacag tagataattc taactaggta |
| 165121 | agactggaaa aataaaaaac gtggcaaagg tactgaaaag aggaaacaaa gctgctctta |
| 165181 | gtatttgttt aatatggttg ggcacagtgg ctcatacctg taatcccaac actttaggag |
| 165241 | gccgaggtgg gaggattgct tgagcccagg agtttgagac tgcagtgtta atgatcatgc |
| 165301 | cactgcactc cagcctgggc aacagagtga tatcctgtca agtataatat ttttgatata |
| 165361 | aatgtggggg cttcccccca aaaaatgctc tcagaattta atgaggaaaa taacctatgc |
| 165421 | caactcttgt ttttttttct ttttcttttt ctttcttttt tttttttttt gagatggagt |
| 165481 | ctggctctgt cacccaggct ggagtgcagt ggtgtgatct cggctcactg aaagctccac |
| 165541 | ctctcgggtt cacgccattc tcctgcctca gcctcccaaa tagctgggac tacaggcgcc |
| 165601 | cgccaccatg cctggctaat ttttttttgt ttttgtattt ttagtagaga tggggtttca |
| 165661 | ctgtgttagc caggatggtc tcgatctcct gacctcRtga tctgcccgcc ttggcctccc |
| 165721 | aaagtgctgg gattacaagc Rtgagccatc gcgccaggcc tctatgccaa ctcttacaat |
| 165781 | gagaSattca gaatgaacaa cggacacata cctgtgatac tgttttgttg cttgaagctc |
| 165841 | aattgtgaga aattaaaaca attccatatt aaatggcctc agtcttttac cctatttctt |
| 165901 | cttcatattt ggggcaattt ctgataaacc acaataaatg atgttgtagg tgacatgctt |
| 165961 | ttatctaaaa gtgtattcag aaatagaccc acacatcatc ttttacaaga gcagggcatt |
| 166021 | tgtctttaaa aaaaattaaa tcatacaaaa gaacttaatg agatccgaat caggatctaa |
| 166081 | atactgtttc taataactct caaggcagtg agtagggaca gtcagccaca gggctcacct |
| 166141 | ggttggttcc tctctctcag gggtcactgt cttttgttgc ctgattttct agtgccttga |
| 166201 | aaactgttgt ttcatatttt tttttctatg tgtttttggt tgttttaaat gggtgggtaa |
| 166261 | atctctctgt tacttcatct tggctggaag cacaagtcaa tttaattaaa tggctcatca |
| 166321 | tttcttgtat agtcctcttt tttgttaata aggagttgtt ttaacacacg ttaatacaat |
| 166381 | tgcttggcct ttcccttaaa gtttgcgtag atttatttta ttttatttta tgttatgtta |
| 166441 | tgttatgtta tgttatgtta tgttatgtta tgttatgtta ttttatttta tttatgaatg |
| 166501 | agacagggtc tcactctgtt gcctaggctg gagtgcagtg gcacaatcat ggctcacgga |
| 166561 | agcctcaact cctgggttca agtgatcctt ccgtctcagc ctcccaagta gctgggacta |
| 166621 | caggtgtgca ctaccacgcc tggctagttt ttgtattttt tgtagataag gggtttatcc |
| 166681 | atgttgccta tgccagtctc aaactcctga gctcaaggga tccgcctacc ttggcctccc |
| 166741 | aaagtgctag gactacagca tgaggcacca tgcctggctt gtttcatttg ttttgaaagt |
| 166801 | aactgttaat tttaaaggtc caatctgtga ggatctaaag agtaggagcc ctgagatgaa |
| 166861 | taattcctga aggctttcag cattctctcc tgctgtgtgc tttgttattg acagctgttc |
| 166921 | aaggtctcac cacctctgga gttttaaccc accgatgtgt ttcataaatt ttaatgaaag |
| 166981 | acagatagag agaaatcata gataggccaa catgattgta ccatatccat gcactggctc |
| 167041 | aaaatgaaaa tctctgtgat attaccctga gttacatttg tggaacacac caaactccgt |
| 167101 | cttctagctt gtctgatcca gctagctaag ggaaatcaaa agtaatttgc aaaatctttt |
| 167161 | atgttctttt atgttcttgg tacattgctc tgatttattt tttaaggcac agaaaattgt |
| 167221 | ccaaatgcat agtacaaatt tgcaaataaa ctacatatag ttatttggac gatggtgttt |
| 167281 | ttctaaggag ttttgtaaag aatgtggttt aacttgccag tgtgtaatgc taaacttatt |
| 167341 | tcttgtgtgt tattatgaac tttatttata atttcatagc agctgtttat tttcattgct |
| 167401 | aaggtacaat tcagatgttc ttggcttaac tcagtattct ctatctcaca aaatttaaaa |
| 167461 | ttagactgat actttgtaaa gattcttcag atgtggccag attattttag Raacagcttt |
| 167521 | agttatccct gataataaat attagggaga gaaaaagaaa ttgaggtctt tgaagaatcc |
| 167581 | ctaaatatca tttcagtcta tcactgtcag cctccctcac tgctttcaga tccattccca |
| 167641 | ggagcattta cagggtggcc aagcttcctc atttgggttc cagctctctt agtttctgta |
| 167701 | gcctactcat tgagctaatg agcagtgtag tggctgagag ctagggagaa atatgagaga |
| 167761 | aatcaaagaa gaccctttct gacttttgcc ctagattgtt tgaaacgaaa taacattaga |
| 167821 | ataatgagga attgaccacc tttcccttgc tgcgttttag gtgtcttttc tgtaccattt |
| 167881 | tattatgtcg tcttaatcat aacagcctac ataaatactt gagtttgggt taggatgaca |
| 167941 | atgaaaaagg gattagaaat aacgtgtact cttttttctt aagccttcag aaggactgta |
| 168001 | aaaacttaaa gatgttaaat gcaaatgcca ttgccattcc aagttaacaa gcataagagt |
| 168061 | tttttgttgt ttatttttgg ggggtgatcc ttaatagtat gtgtttttct tttcagctac |
| 168121 | caagtcagga ttgttacccc caccacctgc gctccctcca agaccttgtc catcacaggt |
| 168181 | aggagacata tttgatttat gtaaactgtt cggtggtagg gaagttgtcc tatcgctgtg |
| 168241 | cttctttgca agggttgatc gccacagatc tggtccatga gattcccaag ctgataccct |
| 168301 | gtgaagagag cagagagtgg tttaatttat tctcatccag ccagctcccc agcctcatgt |
| 168361 | ggaagagaga aagtttcYtc aataagccac caagcataca tatactagtt cactcttggc |
| 168421 | tgtccagggg tgcgtctgtt ttcatcccca gagaagggta gaagaagggg cctctctcta |
| 168481 | attggaggtg gagacatggt ttttctgtat agtatgtgca atctgggtgc ctatattttc |
| 168541 | gtcagtactt taggagctat gttaattcta cataaatgaa aaataaatgg ttatcaacat |
| 168601 | ggggttagtg ccgtggtttt ggcctatggt gctttctgta ggatggtatt tttcctcaga |
| 168661 | acaaaaagct gagatagcag gagggggaga acatgtcacc aagatctgtt tcacccctaa |
| 168721 | attaattctg ccttatttac atgtgccact atggccttat tcatcaacac tttgcagagc |
| 168781 | tgaaaatgag aagcaaacag tggagtgtgg tatatggtgc tacagatctt tccttttgtt |
| 168841 | aattgctgct gaagaaactt tctatcttta aagacccttt ctgatttttg gcagaaaatt |
| 168901 | ttgggaaatt tctctgtaca cagtccctgt ttctacttca ttgttctggg tggttcagag |
| 168961 | ctatccttcc atacggggac atctcaggac tcatgagtca ctgaaaccac ttttaacaca |
| 169021 | tgcttgtcct tctttcagtg aatgaaatgg tttttagcat ccactcaacc ttgagatgca |
| 169081 | aatactatga gatattggct tcctgcttta gtgcgaaggg taataaaaag cccatttctt |
| 169141 | cctgacctgc atataaacca gcgagaactc ctttccagga tgtaacattg gccttaagag |
| 169201 | atattggttg aaccgtcaac aagtatttat tgagtccctg ctgtatatct aatgccaaac |
| 169261 | cttagttttg ccctaagcct ttcattggct gtaatttctt ctcattcaaa ttattagctg |
| 169321 | tgtctgttga gcatctccaa taaatcaggc attgtgctaa gtgctttcag tgcattattt |
| 169381 | aatcctcaca aaacccctct gaggtagata caggtactaa tattatccct tttttttttt |
| 169441 | taacagataa ggaagttgaa gtatatgggg gctaagcaag ttacccaagg tggcatagct |
| 169501 | agtaaatagt agagccagct cccaaattgc aatctgtctc ctaaaggctc ctcctgtcac |
| 169561 | catcactgga gtctgagtgt ctatgtgtac agccagacag cagatccatt acacttgcaa |
| 169621 | tggaggcttt catcacaggc ccctaaggag gaccttgatg agatgaggct cctgctccac |
| 169681 | accaacagca aacctggact tggtcacaac aaaatgagca ggcatttgct agctgctagt |
| 169741 | tcaagcctgg ggcttgagag ccacctggtt gaatgcatgg aagacctgtc tgaattgact |
| 169801 | tttgtaacca accttctagc tttctcttcc tgcacaaatg ccagtagtta ctacccggtc |
| 169861 | ccaaaatatg ctgctgctat cagcagtctt catctgagct gcttgaggtg ggcttcttgc |
| 169921 | ctgcttttgg agcttagtca ccaaagatct gactggggga tctgggaatt ctctagtcac |
| 169981 | tgttaatgaa tgagtctgct tctttgtcta ccagatttcc catcacaata taaacagtga |
| 170041 | gcccagtact cagcctatca gctgcctaac tatggagatt tcagatttca ggaacaggtg |
| 170101 | tgtaagaaat ggaggcagaa gatccacatt ccatttctct cccctattcc ctcctttcac |
| 170161 | ccactgtaag tgggacacac atattcagat atatgaagtc ctgggcaata aacacatggt |
| 170221 | tatatttagt ttttgtgcca tgtcaagttg gtattgaccc tatgagcgtc ttactaacca |
| 170281 | ctttgtatgg gacacctata tattctagtc tttcccaaac tgcttgtgat aaaacagtct |
| 170341 | ctgggagata ttaatagggc tctgtggtac agatttgcag actgctgccc ctctacccct |
| 170401 | ctgggagagt cgctgtgcaa gtgagctcac taaaggcttt ggaggcctac agtaagtaaa |
| 170461 | gctagtaaat tttgttaaag ccagcaattt cctagcttct gtgaccattg atatctttat |
| 170521 | ttataaaata cctgttagga tacaaaatca gtgtgcaaaa agcacaagca ttcctttaca |
| 170581 | ccaagaatag gcaagcagag agccaaatca tgaatgaact cccattcaca atcactacaa |
| 170641 | agagaataaa atacttagaa tacagctaac aagggatgtg aaggacctct tcagggagaa |
| 170701 | ctacaaacca ctgctaaagg aaattagaga ggacacaaac aaatggaaaa acattccatc |
| 170761 | cttatggata ggaagaatca atatcgtgaa aatggccata ctgcctgaag taactttttt |
| 170821 | tttttttttt gagacggagt gtcgcactat cgcccaggct ggagtgcagt ggggtgatct |
| 170881 | cagctcactg caagctctgc ctcccgggtg cacgccattc tcctgtctca gcctcccgag |
| 170941 | tagctgggac tacaggtgcc tgccaccacg cccggttaat tttctgtatt ttttagtaga |
| 171001 | gacgggattt aactgtgtta gccgggatga tctcgatctc ctgacctcgt gatccacctg |
| 171061 | cctcggcctt ccaaagtgct gggattacag gcatgagcca ccatgcccgg ctgcccaaag |
| 171121 | taatttatag attcaatgat attcccatca aactaccatt gacattcttc acagaattag |
| 171181 | aaaaaactgt tttcaatttc atatggaatc aaagaagacc ccatataacc aagacaatcc |
| 171241 | taagccaaaa gaacaaagct ggaggcatca tgctacctga cttcaaacta tactacaagg |
| 171301 | ctacagtaac caaaagagca tggtactggt atcaaaatag acatatagac tagtggagca |
| 171361 | taacagagac ctcagaaata acaccacaca tctacaacca tctgatcttt gacaaacctg |
| 171421 | acaaaaacaa gcaatgggga aaggatcaaa ggtcaaaata aagggctcta ttttaaagtg |
| 171481 | aaatcaggtt ctgataaatt tatcatttgc cctagataac attgttttaa gtttatcaag |
| 171541 | gttgaatatt caagtggaat gcttgttttg tgtaatgtat ttttcctttg ttaactttgg |
| 171601 | cagtaccttt tactgtatgt tccctagtta tagaatcggt tcatagattg ctcagagcta |
| 171661 | ctcatatctc tggattcatg tattatgaag aaacaattct gtttcagacc agcttgtgtt |
| 171721 | gattgaatct caagttttgg aagttctctt gtgaggattt cttaaatttg aaaactacta |
| 171781 | ttctcaagta gttagggacc ataggcacac actaccaaac ctggctttcc aggttttaag |
| 171841 | tatgtaagaa aaatgagttt gtacatttgc aatatgcata cagagatata taaagattgt |
| 171901 | ttctacatac accaaaagca tatattcttt tatcaaatga gagagatgaa gtataggttt |
| 171961 | tcagcataac atgcagttta gctggctcat acttattcaa gcaccactgt gatatgaaca |
| 172021 | tctggattaa gttatacaaa tggggccctg gagttaggaa aaggaataag ttaaaagaaa |
| 172081 | catcttttta tttttcatac tataggtaag atcaagaaga acctagtgac tatatacaaa |
| 172141 | attaagtagt ttgtgtttca cattttcctg tggcaagact agagactagg gcatggtctt |
| 172201 | ttggctcagc tcagaagagg tgactttttt ttgaaaaaag acttatttat agcataaata |
| 172261 | accaaaaagc aattaaagta aactatttca tatagagcag tgcattcttt tgtttgtttt |
| 172321 | tgttttgttt tcttaagacg gtctcgctgt gtcacccaga ctggagtgca gtggcatgtt |
| 172381 | cagggcttac tgcagccttg acctcccagg ctcaagtgat cttcccacct cagcctaaag |
| 172441 | tgttggggat tacaggcgtg tgctactgtg cccggcctag agcagtgtta aagtgagagt |
| 172501 | taaatgtatt agcccactgt ttgtatcatt ttaataaatc cagtaaattg ccatctgtag |
| 172561 | agtttgtgtt tattcttttt tttttttttg agatggagtt tcgctcttgt tgcccaggct |
| 172621 | ggagtgcaat ggcgcgatct tggctcaccg caacctccac ctccccggtt caagcgattc |
| 172681 | tcctgcctca gccttccgag tagctgggat aacaggcatg caccaccacg cccggctaat |
| 172741 | tctgtatttt tagtagagat gggggtttca ccatgttggc caggctggtc tcgaactcct |
| 172801 | gacctcgtga tctgcccact tcagcctccc aaagtgctgg gattacaggc atgagccacc |
| 172861 | gtgcccggcg agtttgtgtt tattctgtag gtcagttctc tgccaaggga ctgtttggta |
| 172921 | cttcagtagc taagtccttt taatagtgtt gatctcatga cagttttctt atggcaattg |
| 172981 | tgatagagcc acagcttcag tgtaactata ataaagaaaa gattttgggg gtgcaagaca |
| 173041 | ttatctttta agttttctat tagcaccaca tttgataaat tggggttccc tgcagactaa |
| 173101 | tgtgctcaga attttcctat ttctgccact aatttctcac cttatatgta acaaagtttt |
| 173161 | cctcactagc aatgtaatga gtactaaaaa tagcactgga aggtgaaagc actgtgggag |
| 173221 | aagcatttga tgaatacctt taagcttaga gagaccaaga agactcatgg aaggggtgag |
| 173281 | atgaccaatg aaattagttt taaagaaaca acagaacttt tttttttttt ttagctggct |
| 173341 | aaaagataca gagctttact gcagatttaa aaaaaaaaaa aaaacccaaa tgcagagaaa |
| 173401 | caggcttgca agaggtcctg gtatgtttga ggagctgcct gagatttgga aagccagatt |
| 173461 | gtagggaaaa tggctgtagt gttctggaag tggccatggg gagcaagcct cctgctcact |
| 173521 | tccagttcca gttctggtag ggatgtggga gagaaacagc ccctaccaaa ggagttctgt |
| 173581 | ctggggagca gaaactgacg ggaggacagg gttttagcct agtggttctc acccttggct |
| 173641 | gcacagtgga atcatctggg ggagctttga acgtgctgag gcctggagcc cctcccagag |
| 173701 | attttggcat ggggtgctga ggtccggcca ctgggaggtt taaaaacctc tccaggtgat |
| 173761 | tctgaagtgc agccagagtt gagaagcact gaattagggc aagaggaagg aatgctcaga |
| 173821 | gaagaggttg agaatatagg gaattttatt gatgagttct tatagggcac aatgaaaggg |
| 173881 | gttctggagt tggggaggca tgaggtcagc ttaggggatg tactcagacc ctatggggac |
| 173941 | aagaactcag atcacaaggg gcctgggagt caagggattc ttgtggtgac tgatgtgaac |
| 174001 | agggctagag gatgtgacag gaaggtcctg ctggaagaac tagtcactgt gtgcctcagg |
| 174061 | tcctgtgtgt actactgtga gaggctagcc atgaacacag atggcctcaa gaggaaacca |
| 174121 | gaacaactgt ggtccattga gttaattctt catctaactt cataatctgg ctagaatcag |
| 174181 | gtcctgaaag tgatattgaa cccaatagga atatggcagt catgctactc tttttcaaag |
| 174241 | actaattttt tagacacaac tttttataaa atactacatt tgttttattt ggaagtaaaa |
| 174301 | actgcagctc acttaaactt aaatggcagt tcgtaggccc tgaatttgtg aaatattcaa |
| 174361 | caccatttta aatactctat ccttttaatt aatatcctgt catttaaatt attctttcca |
| 174421 | caagttaata cacagttggt gattcaaaat agtaaattct tctgggagaa attaagagtt |
| 174481 | ttagaccaca catacttttt tggggcaggg gaaggaattc ctggtgatta tgtaagtctc |
| 174541 | ttacagggct atctaatcca tttttaagtg ttaattttat taaagcataa catacatact |
| 174601 | gaatctattt ttaatacata ctaaatcaat ttttaagata gtaaaatgaa gtataggttg |
| 174661 | agtacccttt gtctgaagtg cttgggacca gaagtggttt agatttcact tttttttttt |
| 174721 | tttttttggg aatatctacg ttacacttac ctagttgacc atctcaaatc tgaaaatcca |
| 174781 | aaatcctaaa tgctccagtg agcatttcct ttgagtgtca tgttggtact caaaaagttt |
| 174841 | tgggaggccg aggtgggtgg atcacctgag gtcagaagtt cgagaccagc ctggccaaca |
| 174901 | tggtgaaacc tcatctctac tcaaaaaaaa aaaaacaaaa attagccggg cggggtggca |
| 174961 | ggcgcctgta gtcccagcta ctcaggaggc tgaggcagga gaatctcttg aacctgcaag |
| 175021 | gcggaggttg cagtgagcca agaccacgac attgcactcc agcctgggca acaagagtga |
| 175081 | aactctgtct caaacaaaca aacaaacaaa caaacaataa aaaaagaaga ggccaggctt |
| 175141 | agtggctcac gcctgtaatc ccaacacttt gggaggctga agtgggcaga tcacctaagg |
| 175201 | tcaggagttc gagaccagcc tgaccaacat ggtgaaaccc catctctact aaaaatacaa |
| 175261 | aaattagcct ggcgtgatgg tacgtgcctg taatcccagc tactcgggag gctgaggcag |
| 175321 | gagaattgct tgaacccagg aggttgaggt tgcagggagc cgagattgag ccactgcact |
| 175381 | ccagcgtggg tgacagagca agactctgtc tcaacaacaa caacagaagt tttgggtttt |
| 175441 | ggagcatttt ggattttgta ttttYggatt ttggatactt aacctgtatt atattgttgt |
| 175501 | cagctttcaa atatctataa gaaaatagca agaaacatta tttaatgtga acaatagaga |
| 175561 | aatgtcactg cttttaaaat tgcatgtttt ttttatcaaa ttaaaactcc gtgaataaac |
| 175621 | accaaacatc atgaaatctc ttgctgtaat atattcaaat gacctcatgt aatatataag |
| 175681 | tcattgaatc accttaagat actgcctgtt aaagcatatt tatgaacttt caaaggtata |
| 175741 | tacaacatcc taatttttac tcatcaaaat acgaatttaa tatgtatcaa gatagtttct |
| 175801 | attcatgcat gtattagaaa atcagaaatt attccaaagt gaatttataa acgtatatca |
| 175861 | cttaagatgc caccttcatt ttcttccaaa gtgttacttt gttctgtcct acagtgtatg |
| 175921 | ctgtagatta gctgaaaatc acaacatgtt aatgagtatt tcagtgatta aggaaagaag |
| 175981 | atataaacca aggctgtacc ttcatttgtg atgatgaaag aacatactga aagctgtacc |
| 176041 | acactgtgct gaatgtagta atatgaatgt gtctgtctgt ggtcttatcc ttagtcctgc |
| 176101 | tactgtgtag ctctctggct ttgtgggaat taagtaagtt agtctcatgg aectatcgec |
| 176161 | ccttctataa agtaagaaga ttggactaga gggatcctac tagtgctaag atgtctgatt |
| 176221 | ctattttgta ttctctgttc aagaattttt tttgtaatat agataaaact tcatgaaaca |
| 176281 | cagttgaggg acaagatagg ttggactaat tttctctgct cctccctgct aagtacaact |
| 176341 | aaaaccccca gaaatactat aatggaaaga ggattttttt tttttgagac agggtctcgc |
| 176401 | tctgttgtcc aggctggagt gcagtggtgt gatcatggct tactgcaccc tcaacctcct |
| 176461 | gggctgaagg gactcagcct cctgRgtagc tgggactata ggtccttgct gccacaccga |
| 176521 | gctaatgttt ttattttttg tagagacagg gtcttcctat attgcccagg ctggtctcaa |
| 176581 | attcctgagc tcaagtaatc ctcccacctc agcctcccaa agccttggga ttacaggtgt |
| 176641 | gagccatcat gcctggccag aacagacttt gaaaggttta aagaggaaag aggactggca |
| 176701 | tgacagtgaa ctagagactt caagtagaca gaagaccaat aaggaaatag aaagcttgaa |
| 176761 | caataccatg gttcaacttg atataacata actatagaac acccagcatc agcacaatag |
| 176821 | acatttttct caagtgcact tggaatattc ttcaggatag accatatgtt aggccacaaa |
| 176881 | acaagtctca ataaatttca aaagattgaa attatattaa acatcttctc tgatgtcaat |
| 176941 | ggaatgaaac tagaaatcaa taacagaagt aaaatggaag attcactaat atgtagaaat |
| 177001 | taacatactc ttaaaaacac aaagaagaaa ttataagtga aattagaaaa taccttgaga |
| 177061 | tgagcgaaaa caaaatcata tcaaaaccta tgggatgcag tgatagcagt actcggcggg |
| 177121 | aaagttttag ctataaacac ttaacattaa aaaagaagag atccaaaatc aacagttagc |
| 177181 | tttacacctt aagaaactgg aagaaggctg ggcacagtgg ctcacgcctg taatcccagc |
| 177241 | actttgggaa accaagacgg gtggatcacg aggtcaggag atcaagacca tcctggctaa |
| 177301 | cacgggaaac tccgtctcta ctaaaaatac aaaaaaaatg ttattctggg catggtggcg |
| 177361 | ggcgcctgta gtcccagtta ctcgggaggc tgaggcagga gaatggtgtg aacccaggag |
| 177421 | gcggagcttg cagtgagcca agattgcacc actgcactcc agcccgggcg acagagcaag |
| 177481 | actctgtctc aaaaaaaaaa aaaaaaaaaa aaaaagaaac tggaagaaaa agagcacact |
| 177541 | agatccaaag ctagcaggac agaaataata aagagtagag tggagataaa caaaatagag |
| 177601 | aacagaaaaa caRcagagaa aatagttgaa aagttgattt tttgcaaaga tcaacgaaat |
| 177661 | tgacaaaact ttagctagac tgaccaagag tataattaag catgtgttaa cctctggtca |
| 177721 | aaataaaggg ctctatttta aagtgaaatc aggttctgat aaatttatca tttgccctag |
| 177781 | ataacattgt tttaaattta tcgaggttga atattcaagt ggaatgcttg ttttgtgtaa |
| 177841 | tgtatttttc ctttgttaac tttggcagta ccttttactg tatgttccct agttgtagaa |
| 177901 | tcggttcata gattgctcag agctactcat atctctagat tcatgtatca tgaagaaaca |
| 177961 | attctgtttc ggaccaacct gtgttgattg aatctcaaga gttttggaag ttctcttatg |
| 178021 | aggatttctt aaatttgaaa actattctca aatagttcgc actgaatgac aaattcaaaa |
| 178081 | ggcaaaggaa atggccatag tgataagtgc aaggagaaga gagggtgaga attagtaatg |
| 178141 | aaataccagc attggaatgg aggagaaaag tgtaagcctg tcccccttcc ttctttttga |
| 178201 | aagaagtRct cagcattttg ctctttatca ataagtgtct ttagaattaa tcctagtaac |
| 178261 | taattcaaaa gaaaRgaaaa gttctatgat gacatttatt ttagtattat aaaaataaga |
| 178321 | acaatacaga ccaccttaat atctaggaag aaggcactgg tcaagggtaa tgtatatctg |
| 178381 | cctgattaga atattaggca tctattaaaa tgattacatg acaaaacagt agaaaagagg |
| 178441 | ggtaacagct gaatacagag gtgtttcctg attgtaccac ataagtatat ggtagacctt |
| 178501 | tgatggtaag gttttctcat ctctgaggat gaccttgaag gtccatggtg catggtaatt |
| 178561 | ggcccctttc cagtggtcgg atttcgaatt acttttacat tagccactgt tccctcccca |
| 178621 | gtagggtttt gagattctta gctgcttagt gaatttctac taactctttt taggggtcta |
| 178681 | aagggatcgc ttaggtgact catttataat ttacctatct tgtcttataa cttgttttta |
| 178741 | taagttttta tattttggga gatgatgtat gtgtagtatt tatgtgaatt gggatatctg |
| 178801 | tgaaggtctc cagatggttc agtcctaaat gtcaagtgcc tactgttaaa tacttggaga |
| 178861 | agactgctaa gtaattggta gaagtttaaa tggctgtttt ggtggggaag ttatgcagtt |
| 178921 | tttcttttaa aaattttgtc ttaaaattaa ggattgggga aaagaaaaaa aagtcctctg |
| 178981 | atatgacaac tagaataaaa agtcatttga agatttacca gaataaaatg acctttggaa |
| 179041 | actactttta gagaccaget ctcagaaata gtgataagac atatattgca taattacatt |
| 179101 | gttacgatga ctctagcaat tgtgaggttc aaagaaatag tgtactgaag tattatcatc |
| 179161 | tagtcatttt ttaaatctat gttctttgga tagtaaactc ttattgccct cactcttaaa |
| 179221 | tagtaattct gctgtgtaca gaattttaga ctgaaagtaa tttttcctca gtattttgaa |
| 179281 | tgcattacct cctcttctag catttattgc tgccactgct ccggattgta ctgtcaatct |
| 179341 | attttttatt cctttggtgc agctttgtct ttcctctggg attgctttta aaagtttatt |
| 179401 | tttactttct gaaggggtgg gttgcccctc cacacctgtg ggtgtttctc gttaggtgga |
| 179461 | acgtgagact tggaaaagaa aaagacacag agacaaagta tagagaaaga aataaggggg |
| 179521 | cccaggggac cagcgttcag catatggagg atcYtgccag cctctgagtt cccttagtat |
| 179581 | ttattgatca tttttgggtg tttctcagag agggggatgt ggcagggtca taggataata |
| 179641 | gtggagagaa ggtcagcaga taaacacgtg aacaaaggtc tctgcatcat agacaaggta |
| 179701 | aagaattaag tgctgtgctt tagatatgca tacacataaa catctcaatg cgttagagag |
| 179761 | cagtattgtt gcccgcatgt cccacctcca gccctaaggc ggttttcccc tatctcagta |
| 179821 | gatggaacat acaatcgggt tttataccga gacattccat tgcccaggga cgggcaggag |
| 179881 | acagatgcct tcctcttgtc tcaattgcaa gaggcatgcc ttcctcttat actaatcctc |
| 179941 | ctcagcacag accctttacg ggtgtcgagc tgggggacgg tcaggtcttt ctcttcccac |
| 180001 | gaggccatat ttcagactat cacatgggga gaaaccttgg acaatacctg gctttcctag |
| 180061 | gcagaggtcc ctgcggcctt ccgcagtgtt tgtgtccctg ggtacttgag attagggagt |
| 180121 | ggtgatgact cttaactagc atgctgcctt caagcatctg tttaacaaag cacatcttgc |
| 180181 | accgccctta atccgtttaa ccctgagttg acacagcacg tgtttcagag agcacggggt |
| 180241 | tgggggtaag gttatagatt aacagcatct caaggcagaa gaatttttct cagtacagaa |
| 180301 | caaaatggag tctcctatgt ctacttcttt cgacacagac acagtaacaa tctgatctct |
| 180361 | cttgcttttc cccacacttt cRgtgttctg ttttgatggc gctgcgatgg gtttaagcgt |
| 180421 | agatttaaat ttgtttatgc caggtgacac ttggcatgca ttttccttct ggaaaatcat |
| 180481 | ctttaatttt aggacattta gtgatatctc tgtgggtttt gcttctctac ttatttcttc |
| 180541 | tgttttcttc tggaactaac tatatttctg tcaacacatt taaaaaaaac tctagtaatg |
| 180601 | tgtctttcat cttttttttt tcttttttga gacagggtct tgctctgtca cccaggctgt |
| 180661 | gcaatgggat gatccatggc tcactgcagc ctcaacctcc tgggctgaag ctgtcctccc |
| 180721 | acttcagcct cctgagtagc tgggactaca ggcgcatgcc accacacctg gctaattttt |
| 180781 | tgtatttttt gtagagatgg ggttttttca tgttacccaa gctggtcttg aactcctggg |
| 180841 | cccaagcagt cctcccatct caacctccca aagtgctggg atgtacagat gtgagcaccg |
| 180901 | caccttgtca ctttcatcta tttatgttgt gatctttctg ggcttgctct ggatgaaatc |
| 180961 | ttcatcttcc aaattaaaaa tcctctgcat aactatatag cctagaattt ttcctattct |
| 181021 | gttattaagc ctctatcact attctttgat tctaggtttt ataatccatt cttttttata |
| 181081 | acaacctgtt cttgttttac ttttccctat ttttgtttta tacccatcta ttctttttta |
| 181141 | ggatctattc tccactttta actttgagga ttctaaaata cttactttaa agttattttt |
| 181201 | aacttgttct atttttgttt ttgtcaggag cgaatttgcc ttttttattt ggctgttcta |
| 181261 | ccatgctttt tggaattttg ttttgtaggc ttattttgag ttggagttca atctcattct |
| 181321 | cagttctctg agctcctcac tttctgccag ttcagtggct gtctctacca actattcctg |
| 181381 | gcatccatgg cctccagtca agagctgatt cttatacttg aaatacagta cttccatctg |
| 181441 | gtgcaggtgt caggggtctt gtagatggca cccaatggtg cttagcccag aaccaggctt |
| 181501 | cagtgttata tctgcttcag ttcctttcct aagtaggcaa cccagagctt ctccacaggc |
| 181561 | ttcatcttgg ttttgcagct tcacaggtag caaagtgata ccttagcctc tggcatcagg |
| 181621 | gaatgagctt ggtaccagtc ccctgcctca tgcgggacat ttatttattt ttatttttta |
| 181681 | ttattattat actttaagtt ttagggtaca tgtgcacaac gtgcaggttt gttacatatg |
| 181741 | tatacatgtg ccatgttggt gtgctgcacc cattaactcg tcatttagca ttaggtatat |
| 181801 | ctcctaatgc tatccctccc ctcttccccc accccacaac agtccccagt gtgtgatgtt |
| 181861 | ccccttcctg tgtccatgtg ttctcattgt tcaattccca cctatgagtg agaacatgca |
| 181921 | gtgtttggtt ttttgtcctt gcgatagttt gctgagaatg atagttaaca gcttcatcca |
| 181981 | tgtccctaca aaggacatga actcatcatt ttttatggct gcatagtatt ccatggtgta |
| 182041 | tatgtgccac attttcttaa tccagtctat cattgttgga catttgggtt ggttccaagt |
| 182101 | ctttgctatt gtgaatagtg ccgcaataaa catacgtgtg catgtgtctt tatagcagca |
| 182161 | tgatttataa tcctctgggt atatacccag taatgggatg actgggtcaa ctggtatttc |
| 182221 | tagttctaga tccctgagga atcgccacaa tgacttccac aatggttgaa ctagtttaca |
| 182281 | gtcccaccaa cagtgtaaaa gtgttcctat ttctccacat cctctccagc acctgttgtt |
| 182341 | tcctgacttt tttttttttt tttttttttt ttttttgaga cggagtctcg ctctgtcgcc |
| 182401 | caggctggag tgcagtggcg ggatctcggc tcactgcaag ctccgcctcc cgggttcacg |
| 182461 | ccattctcct gcctcagcct cccaagtagc tgggactaca ggcgccYgcc actacgcccg |
| 182521 | gctaattttt tgtattttta gtagagacgg ggtttcacca ttttagccgg gatggtctcg |
| 182581 | atctcctgac ctcgtgatcc gcccgcctcg gcctcccaaa gtgctgggat tacaggcgtg |
| 182641 | agccaccgcg cccggccgtt tcctgacttt ttaatgattc atgtgggaca tttaaatccc |
| 182701 | tgttattagg aactctcagc tgcctctgtc tgcttctgca ccctctgcct gcttctgcac |
| 182761 | cctctgcctg acctaccact ttgtgatttg gccccacaga gacctttatc ttgcttctga |
| 182821 | gtgggactga gtcttttaaa ttttaacagt tcatgctaac actctcacat ctgtcattgc |
| 182881 | atatcatttt atgtaagggg aagttgagat tcagagagca gaagttaact acttgggcct |
| 182941 | cgcagttggt aagtaagcca gcctgctgta gaagaaaagg gctggaaaag gtttctgggg |
| 183001 | aagtgctaga aatttaataa gcatgtagag agagaaaaat cattcattat acatcacaaa |
| 183061 | aattaactgt agatctgcta agtaaaagtt gattaaattt ctacactctg tgcccaccaa |
| 183121 | taattttcta aatgtgaact ctttcctaca ttaatctcaa atgcttattt ttaaagtgcc |
| 183181 | ttccccagtg cagacttaca ttcatcttag ccaaaatggc cttcacattt ggtctgtggg |
| 183241 | acatatttga tgtagcaaaa ggccaagggt aacccttatt ctacccccta aaagggaaca |
| 183301 | gaaagagaac ttttgcataa taaataatta tttatgcatt tcaaaattga ttttaagagt |
| 183361 | acacaaacca tagaggcttt gcgattcttc atgtatttta ttaatttatc aagcacttca |
| 183421 | acagagtgcc cgatgctctt ctagatgctt tacaaatatt aactcattta agcctcacaa |
| 183481 | caaccacgtg cagtgggttc tattgctatc ctaacattgc agataagtaa acaaggcaca |
| 183541 | gagaacctca gcaacttgct tgaggtcagt gacagaaaga aagagttaag gctatgattc |
| 183601 | ctaccttggc tatttagccc cagtgtacca gctcctacta tactactatg ctgtgctgtg |
| 183661 | aaaccacgtc tcacaattat tttattaaca atcaaactga cacactatct ttgtcaggga |
| 183721 | cacattttat ttcatggaac tagttaaaaa ttagctttaa ctcacttctg gtgacttgtt |
| 183781 | ttctttctta ggttcttcct cattatatta tgcccatgct attgttatat aaagctcact |
| 183841 | ctgctggtat cattcttctg gaagatgcca taaattcatc taatcatttc atcagatgtt |
| 183901 | aacggaatgt gctcactgta ggcccacagt ttgctgagaa ctataggagc acaaagattg |
| 183961 | tgatgtgatc cttatcctgt agttccttta tcctgcgtca ccctctcaca gcagtcatgc |
| 184021 | ccaacactat ctattctata gtctctcatg gttcttacaa ctgagtcatt ttctgtgtgt |
| 184081 | ctcatttaaa cccatttctt aatgctttat tgacacaggt tgtaagcaat aaactggtca |
| 184141 | ccatgtttct aataactacc aatatagccc agttttcccc tggggggctc agttatggtg |
| 184201 | gttttctagg tttYtgagca atggaatcct catttctctt ctctgctgtc ccctatcgca |
| 184261 | ctgtgacttt tttttttttt tttttttttt tttgagaaag agtcttgctc cgttgcccag |
| 184321 | gctagagtgc agtggcgcga ttctggctcg ctgcaacctc cgcctcccag gttcaagcaa |
| 184381 | ttctcctgcc ttagcctccc gagtagctgg gattacaggc acatgccacc acgtccggct |
| 184441 | aatttttgta tttttagtag agatagggtt tcaccatgtt ggccaggctg gtctggaact |
| 184501 | cctgaccttg tgatccacct gccttggcct cccaaagtgt tgggattaca ggcgtgagcc |
| 184561 | actgtgcacg gcccacactg tgactttaag gaagcactcc tgagggggtc aggggaggag |
| 184621 | cagatgtgga ttgggaggat gcccaaatgt gctactgtgc taatgggaaa ggacaatgga |
| 184681 | ggacaaccag gggttgggct cagggaggca gcttgacaga ggatttaaga gcatgggctt |
| 184741 | ggggtttcaa atcctatctg cctcttactg gctttgtgac tttgggcaag ttagtctctc |
| 184801 | tgtgcctcaa tttcatcatc tgtaaaatgg ggatcatggt agtatccatt ttcatagggt |
| 184861 | tatgatgatt aaatgagtga agatctctaa agcacttaga atgatgcctg gtgccatagt |
| 184921 | caaccctcaa taacatttag ctgttataat tatcatcacc acaggcctag tgaaacagag |
| 184981 | gtggggtcta gttggcaaac acagaacaga ctccagatag ctagaaatta gttgcaacca |
| 185041 | gcccagaatg agtgctgaag tggctcgcta aagcacttgc agtcaaagca cattgttacc |
| 185101 | ctcatctgtg acctttcatg ggctgtctct ctgcctatgt caggggttgg caaactatag |
| 185161 | cctacaggcc aaatccagat ggtcacctgt ttctataaat aatggaaatg tgaaggagaa |
| 185221 | tcaattggca agaggcacgc atagcgcatc ttcaaccttt tccatcgggg aacatgagtc |
| 185281 | ttgagaatcc tgccatagat caaaagaatg gcaatgagaa gaaccagtgg acaggtggat |
| 185341 | aggcctcccc atcagccttt aaccctcctg ctcttatctc aaggatcata tgcccgtgtc |
| 185401 | tgtggtttct gatttttcct tgaggacctt tgaccgagat gatctgtctt tcagacttta |
| 185461 | ctagcaaaat tctgagcagt atcttttata caactggcag cttcaaaggc ttgcagtggg |
| 185521 | taggcctccc caactctgtt tcgcaagata attaaaagaa gccagactcc ttcatccttg |
| 185581 | aaggagagga agaagaaaac aagcctgtcc agagaaaatg gggtgaagga atttttctcc |
| 185641 | ccaacttcct cctaaatacc ctgctctgcc catcaaattt tgtaagtatc acccagtggt |
| 185701 | gtgttggtaa accttctccc ctgcggggtg ggggaaagcc ccaatttgca gccgttgttg |
| 185761 | ctgtctgtgt tgtgaatgct cccaccatgc tgactgcaag tggagctggg aagagatgtg |
| 185821 | taccatcggc tcactaacac tagcatgctc tgtggtagac agcttccctt gcctctgaat |
| 185881 | ccttgtgtca ggattctacS tgccaggcaa caagagcttt ttttctttat gtatcatgcc |
| 185941 | agtgatttta gtctagggca ctgttttagc ccaatcccag aatgtgctat tagaaatggg |
| 186001 | gccatatttt tgaagttgct aggggcaaaa cttccctctt gtttatgttt aacttgtgcc |
| 186061 | actaggtggc actttaagga cattacagga aatcggctct actggatttt taaaactttt |
| 186121 | ttttttttga gacggagtct cactctgtcg cccaggctgg agtgcagtgg catgatctca |
| 186181 | gctcactgca acctccgctt cccgggttca agcaatttag ctcactgcaa cctccacttc |
| 186241 | ccgggttcaa gtaattttcc tgcctcagcc ttccgagtag ctgggactac aggcatgtgc |
| 186301 | caccacaccc agataatttt tgtattttag tagagatggg gttttgccat gttggccagg |
| 186361 | ctggtctcaa actcctgacc tgagttgatc tgcccacctc ggcctcccaa agtgctagga |
| 186421 | ttacaggcgt gagccactgt gcctggccca aaacactttt tattgaagta taattgcata |
| 186481 | tagaaaagca agctgcacat ttaatgtgca actcaatttt cacaagcaga acacatgcat |
| 186541 | ataatcagca cccaaatcaa gaaacagaat attactagca ccccagaagc cctccttgtg |
| 186601 | ctgttttcca gtcgctatct cttattctta gggttaccac tattctgact tctaacagca |
| 186661 | taaattagtt tgacttgtgt tctactttat ataagtggaa ttatgcggta tatactctgt |
| 186721 | gtctggcttt ttattaaatg atgtttgtgt gatttattca tactgttgtg tgtaattgag |
| 186781 | atcattgctg tatagtattc cattgtatgt gcaaatataa cactgttttc tactattgat |
| 186841 | ggacattttt gtacttttca gatttgggca attatgaata gtgctgctgt gaacactcgt |
| 186901 | atccaagttg ttgagtggat gagtgttttc atttctcttg gatatatacc taagagtaga |
| 186961 | atttctaggt catggtgtag gcatatgttc agctttagta ggtactgcca aacagtttta |
| 187021 | cgaagtgttt gtactgattt acactccatt aatggtgttc ccactccagt tccatgtcct |
| 187081 | catcaatgct gaatttattt tatggatggt tcctaactct taacataatt tttatagctc |
| 187141 | cctttctggc cagctatttc taattgaaaa attttaaagc cacattattc tgatggacat |
| 187201 | ggctcatgca ggaagggcta tgtcaaccca aaaggggaca tttatcacag tctaagtgtg |
| 187261 | aactatcagt aagtcggaaa ggatgttact ctaatactcc atcacactga tacacagagc |
| 187321 | aaagaaacca ggaaaggaat tcagattgta agtgtgataa aatatcggtt cataggccag |
| 187381 | ggctctgagg cattttgaaa tttgtatata catttgttat tattgccagg cctatatatg |
| 187441 | tcagagattg gggcccaaca gaaagaattt ctaaaggcct cataatgaag tgcctttcaa |
| 187501 | ttgtgctgct ggtgggtgcc tgttttacta agggtctctt tggtttggca agaggcatgc |
| 187561 | atggggcatc tttcttcaac ctcatccctg gggagcgtga gtctctagag aatcctgctt |
| 187621 | tagatcacct tacctgtgag gctcatgttg ttgactctgc agatggtgtt ataatatggc |
| 187681 | tttaggtatg tgatatagag agtacagttc agacttgact cagatttctg ttccattact |
| 187741 | agaaattgga catagaagaa caaaacccag gtcatggtca aaatatttgt cttttagacc |
| 187801 | aaaataaagt gctcatagtc ctttagttgt aacagtaagt ctgtctcatg gatgttgtcc |
| 187861 | ataatctaat gggttccaag aatccccaag gcattctgcc atgtgactct tgaatccagc |
| 187921 | aactagttgc ccattatagt cactggtacc cactttcacc aaagttgttc ctgagcccct |
| 187981 | ctgtcagtgt tttcctgagt gtctccagtg ttggattggt ctaccgaggg gggtgcccac |
| 188041 | cagagatgtt cactgcacac tgcctgagat atccatgtta aagcagccgt gaaacatgaa |
| 188101 | agtctgaccg ggcttttatt tttaaaattt tctcccccca atattagtag cggtaatatc |
| 188161 | cagtggcaca gatttagact gtggatagcc tgctttgtgg gatctgctta tattctaaat |
| 188221 | tggagccagc attttctcag cctaggtttt catctgtttg gcaaggagga aaatgccctt |
| 188281 | atgttggatt ggctgctgct tgcagtagga ggaactatgc ctgggaaacc aatcataatt |
| 188341 | ttaatcctaa tgataatagt agctgctgtt gtcaaacagt tactgtgtaa ttgtgcttag |
| 188401 | cacactagac agatattttg cttaatcttc acaatattgt gagttaggtg aattatccca |
| 188461 | ctttgcagat gaagaaattg cagctcagag agactaagca tgtgaccagc tcacacaatg |
| 188521 | agcaactgca aagcctgggt ttgactccag agcctatatc ctgtaactgt tctgtgctct |
| 188581 | gtgggtgcag ttaccaagga atgaattact gacctcttct ccagtggaac atgaaacagt |
| 188641 | attgggttca ttgctggctt attccacaca ttgattagag gacctgttgt ttgctggcat |
| 188701 | gcagcttagg tatcaagaaa atgataggga agcaagacag atcctgccct ccaggtgctt |
| 188761 | accattcgtt tatcatgttc tgtctctcac aggtgacttg agtcatggat tttggtaatc |
| 188821 | attcttgctg agaattcagt ctcttggacc ttcttgaggc tctatttacc cctctctctg |
| 188881 | agcctggttg gggaatgatg gggtggaaca gtggtgtgct gggccattgg ttctacatct |
| 188941 | tctgtgactg ggtttagcaa atggctttct gttgtgttct tgcctagcgg agatggggga |
| 189001 | caggtgaaaa ctaggcacca aatgacattt agaatactga cagaatgctg gaaaggccct |
| 189061 | ggaggcagca gcaggattgt cttcttgtct gcctagaatt tagctgctgc ctttgtgatc |
| 189121 | cccattcttg gaatccacct ggatccttgg atgataaagc ctgagttcgt ttttctgacc |
| 189181 | tgccaatatt gcaaacctaa aaaaaaaaaa gttacctatt atccacacct tggcaaagtt |
| 189241 | ctacagatgg acttcttttt tttttttttt tttgagacag agtcttgctc tgtcacccag |
| 189301 | gctggaatgt ggagtgcagt agcgagattt cagctcactg caacctccgc ctcccgggtt |
| 189361 | caagtgattt tcgtgcctca gcctcctgaa tagctgggat tacaggagcg tgtcactacg |
| 189421 | cccagctaat ttttgtattt ttagtagaga cggggtttca tcatgttggt aggctggtct |
| 189481 | caaactcctg gtgtcaagtg atccacctcc caaagtgctg ggattactag cgtgagccac |
| 189541 | catgcccggc cagactattt tttaagtggg atttgacttt ttttttggtt gcaaatttga |
| 189601 | cttttaaaca aaatcttact gctgaagatt ttgcccagct gcaaatattt atctatagaa |
| 189661 | ccatgtggca tataccccca ttccctctat gatgaatgga tgagaaaaca attaagaagc |
| 189721 | ctagttagtt ttgcaagagc acctgtggcc ttgcccagac tagggccttg gagacttggt |
| 189781 | tttctcatct cttaaatgaa gagtttggac catcagtggt tcccagttga tctgggctgg |
| 189841 | ctacataaaa atcacttagg gtgcttgtta aaatataagt ttctaatgcc ccatccctaa |
| 189901 | gggattctga tgagggaagt ctggaatagc atctacaaat ctgaatttat aaaaagttcc |
| 189961 | ccagataatt atcctgctct ggttatattt gggagccact gggctagagg gtctagaaca |
| 190021 | aacataatta agttagggag aaacttctga tgtgcttgaa gatttgggga atacctatat |
| 190081 | gcctggagca ggttccatat gggcgaacta acccagttat gttgcagagg taaattggag |
| 190141 | ccagaggatg gaagaccttt aactggctga gggattgtgt tgtgtgccat aggctttggg |
| 190201 | gagccagtga agggctttga gaaaaagcat tttgattaga tggtgcttta ccagattaat |
| 190261 | ttctcagtgg tgtgcagggt ggatcagagt gcactagaac tgaaggctgg aaaaagaccc |
| 190321 | agtgaagtct ttgcagtggc ccaggtgata agtgtgacga gtacctggac tcagatggga |
| 190381 | ccagtgggac ctttgtaaag ctaacctgcc tcagtggaac tggaatctga gacatactgt |
| 190441 | tttaaactcc ttaaaaaatg cataatttat taatgtcatg ttcatctgcc taaactatta |
| 190501 | taattaacta tttttattgt gtttttgtcc aagaggtttt aaaggagccc tgtaaataaa |
| 190561 | ttcatgcaaa gaagtagttg gcagcagcaa cagctgctac cgcagggaga agggaccccc |
| 190621 | ttctggccag gcagcaacct cattctcacc ctgtctccat actctaagcc aaagcgagag |
| 190681 | gggaggggag tgcaacaggg gcccctggag ttggatctgc caaccacagt ggagaatgca |
| 190741 | cctccccaag ggttaggtgt ggaatatggc gtttggcatt aagtcaaata gaatattctt |
| 190801 | cctttgttcc tctaaaatcc agtctatagt caatgtttct caaagtatgt tccacagaac |
| 190861 | attagttctt caggatgtta atagaagtta catgagaata aggagatgaa accaaataaa |
| 190921 | tttgggaact gatgggctac gcaaaggcaa gtgacttcct ttcagaattt cagagtgtgt |
| 190981 | aatgttctgt gtgctcagtg aatctccaag Rgggagatag accaggcagt gtttcctaag |
| 191041 | cttatctgta gattcttctt ccccctaggc caggggctga caaattatgg ctcataggcc |
| 191101 | tcatctatcc caccactttt tttttgtaaa taaagtttat tggaacacag ttgtgcccat |
| 191161 | tcatcgtatt gtctgtagct gctttagcac aacaggagag ttgcagagtg gctacagagg |
| 191221 | tagtctggcc ctcaaagcct aagatattta ctatctgccc attacagaaa ggtttgctaa |
| 191281 | tccctgctct agacaattca tggaactggt gcttcctggt accccctttc agagatgcta |
| 191341 | ctagaagaag atatacatgg gttgttctag cactgcttta tgataaatct gcctcatttt |
| 191401 | attctgtttc catactgcca tcattcctgc ctcttgcttt aaactacttt atctctcaac |
| 191461 | catttaccca ttatccagga tccaaacctg ggttccatgt gtccttatct tttcttgcac |
| 191521 | tcacaacatt ccaaaaatgg ccaagggtgt cttcaatttc cagtctgggt agctcttaag |
| 191581 | tcagcccgaa cccattccta ccaacctggc ctacatgacc gtcatctttt ctttttcagc |
| 191641 | attcgaatta ggttcctttt ccccagatat gagttacctc tagtcctttt tactcactga |
| 191701 | atctagaata acctttgtaa aatacaaatc tagtcagatt gattccttac ttaaaatcct |
| 191761 | tcagtggtcc ctgtggccct caggataagc tcagatcctc aggatggatg tgtgtcagtc |
| 191821 | tggttgggca aacagaaact actcctggta tttcgtactg gaagagattt aatttaagga |
| 191881 | attagttact taagaaaagg agtagatatt tccaggcttc tgcctgctgt ggcatagaga |
| 191941 | agggcatgga aatgggggat ggtgttgagt gtttagcaaa tgacccagaa caaaccaggt |
| 192001 | ctgggaaacc atgtgttatc ccctgctgcc cttctccagc ttcagctctc ccatttttct |
| 192061 | cctgactcct cacccttcct ccaccctgca agtccccaaa ttctagatat atcaggatgc |
| 192121 | ctttttgtcc ccccagaaac ttaaaacaac tgacatttac gaactcacag tctctttggg |
| 192181 | tcaggaactt gggagtgact tagctgggtg gttcggggtt acatcctctc atgaggcttc |
| 192241 | tgtcaagatg tcttccaggg ctgcagtcat ctgaaggctt aactggagct gatgatccac |
| 192301 | ctctaagatg gctcactcac agggctgttg gcaggaggcc tcacttcttt gctgtatgga |
| 192361 | ccccttgtta ggactgcttg ggtgtcctca caatgtggca gctgactgtc tccagaacaa |
| 192421 | gtgatatgag agtgagagat agagatagaa acagagacaa caaggaggag catactagag |
| 192481 | tgctttcagt cccctctcct gggagtacgt tgcatagtgt ctcaggtcag cttccctaga |
| 192541 | agcagaaccc tgagatgagg attcttcaaa agtgatttgt tgaaggagtg ctctttagga |
| 192601 | aaaaacctag aaggaggaga gggaagaaaa gtagggaagg ggaaggagcc aagcaatgat |
| 192661 | atagtctcag ataaaatctg accttggcct gatccacaca ggcaggattc tggtacataa |
| 192721 | ttgcataata gagttaaccc tgtttgagac aggggactgg ccttttatat ccttctctaa |
| 192781 | gtcagtcatt ataggctgct ggaggtgggg gtgggaggta ggctcccatt agttgagaat |
| 192841 | aagtctctag agaagagggt ggctgtgagt cctcagtagc taacactcag agcagctggg |
| 192901 | ggatggtgtg ccagccaggg aaaggtgacc caggtggggc caacacagca cctactacag |
| 192961 | tgggcctccc cRtacccatg tcccaagcac ctaccatggt ccctgacata ccagaggtac |
| 193021 | gaaaaaaata tctgtgtaat tgaataaatg aatgcataga aatgcttagc ctctctggac |
| 193081 | ctgtgtccat ttctgggaag tcagctcagc agggatcata ttgtcccatc aaccatggga |
| 193141 | atggtcgaag acccaggaca gtttgccaca tcatgagtgg cagcactgtc taagtccagg |
| 193201 | attttcattg caaaattaga gtgaaagtat atttgccatt tgtgaataag gaagtattaa |
| 193261 | tattacaata ttatatattt ttaaaaggaa ctttagctca catttcagtg agctctttgc |
| 193321 | taaaagtaca ttttcctggt ttgtcacatc ctgactgggt acatgttgtt acacatggaa |
| 193381 | ccgtgagggc agaactgcca tttgttatat taattgtaat ccatatatct gtttttttcc |
| 193441 | cacaagaatc agtcactgga tttcagttca gaaaaccaaa actgtcggac aaagccatat |
| 193501 | tatcgtgttg gcagagttgt tccccatgac cccaaataca cagaggagga agagaaccac |
| 193561 | tgaggcccct tgttttcttg gattatcatt cttccaaagt tggtaactaa gaaaagtctc |
| 193621 | aagaaatagc aaagaaacct ccccacctta ttctgctttc cacatcccaa gcctaacata |
| 193681 | agcctcaaac tatttttatg aaggaaagct tattgtgaag agggtctttt acatgcctaa |
| 193741 | atctcatctt tagtttttct tatcacacag ccggttatct ttgcccatct cttttcctgg |
| 193801 | gataatgtct ggctctgtca aggctgtgcc ttattctggg accttcctaa tgcctgtgtt |
| 193861 | ccaggtccct cctgagattc gctcctccga atgcttgata gatggatcaa tttaggggat |
| 193921 | tgatggtcag atttagcctg acataacacc atgcagtggt ctgttaccag acatagagct |
| 193981 | ggaaaaccca ggcccttgca tacaagttca ggaaggtaaa tttgcaaatg aaaaacattt |
| 194041 | taagacacat ctcacagctg cttcctcatc ccccacccta ttgtttgtgt ttttagagat |
| 194101 | gtttaagaga aaaggaggca ataacaaaga cgctctgctg tgctttggaa tcctcattta |
| 194161 | tgttgaaaaa gggtccattt atcatctcct tggatatttc cacaaggaga ctttgctcat |
| 194221 | ctcaccaggg aatggagtgg tagagcaaac acttctctgt ccacctgatg atggaggaga |
| 194281 | accaactaac tgaaaggcag agagcctgaa attattatcc tctctctgcc tgcctgctgc |
| 194341 | aagatattgc tgcatacctg tggtccctgc aggaggctgc atgcgtagac ttgggccaga |
| 194401 | acatatatta ccttccaagc aatagatttc taggtaaaag gcaagaaggg gcctcaggtt |
| 194461 | caagttgcat tcttcagtgg ggttcacagg aggccctgca ttcagggggt tactgggccc |
| 194521 | tccttcagct gttaataaaa ctgacttttg gctcatagag cttggagtat gcgtccattt |
| 194581 | gtatgaacct ccagggaaag cagggtgagg gcttgtgttc cctagactcc tggcagtgaa |
| 194641 | tggctcctgg acagctttgc ttggttgttt cccaggtact tcaaactcaa tctgcctaaa |
| 194701 | gatgagttcc ccctcactcc tgcttctcct gtgtttccca tcttggggat tgaccttagt |
| 194761 | gttcttgtgg tagccctggc tgaaattatg gatgttggag catgggaggc cctttgtgaa |
| 194821 | cttacttttg ggtctttctc accattccca cacattgctc ttttagccca ggcctcagct |
| 194881 | gagacateac ctgctcctga gtctttgccg acccatgaga ctgggtgctt ctgtattccc |
| 194941 | catacttgcc ctccacagtc cactgaagca agttggcttt gtcacttccc tgttgctccc |
| 195001 | actagacttc aagcttctct taagcaggat ctgtatcttg ttcatccttg agtgccagtg |
| 195061 | ctagcacagg gcctgcctgt agtagctgct taataaatat gtcttgaatg agtaacactc |
| 195121 | aggcctgaga atacNtgaga ctgtatggca tagtggttat aagcttagct ttggaaatca |
| 195181 | aggcctgtgt tcagactctg ctatttgtgt gattttgggt gagttcctta acttcttgat |
| 195241 | cttcaatttc ttcaccaata agatggggat aataactact tcctagggct gtgatgatga |
| 195301 | attgcaatga tccatgcaaa gccctttgat caatgtatga cacatagtaa gtgttgtgtg |
| 195361 | catcatagta cctattagta acaacaacaa caatatagtt atcgttaatg gaagggagaa |
| 195421 | agctataaga actagttgga actagaagtc aggaatattg agtgcttgca tagtggattt |
| 195481 | agaggagatg gggaattgct tgggataaaa tgtaggaaca gatgggatta ccctgggaga |
| 195541 | gaatgaacag ggagggaaaa atgggtggag caactaaaga cagaaccttg agaaatccta |
| 195601 | catttgaggg ataagaggag tcaaaacagg aaaaagagtg aatgactaca gttaggagat |
| 195661 | tgcagcttct cagatgccag gggcaggagt ttaaaaaaga tagtcaaagg tgctatactg |
| 195721 | ggcagaagat caatgaggag aagacgagaa gaggtagcca taacgtgtta aatgggggta |
| 195781 | tgttagcgcc cttgggaact agccatttca gtggagtcag aggaagaagc catgctctga |
| 195841 | aggaggttag agaaggccac gttagggatg aagtgggcat actaagtcag tttgttgtgc |
| 195901 | tacaatgtgt gtttctgtaa tgaggattat ctgtgggtca gtagatgact ggtgtcagta |
| 195961 | tgatgtagaa gtcacattgg ttcatatgta gggttcattc tcagcacatg aatgttttgg |
| 196021 | catcactggc tactagtggc tgaaagcaga ctaacacacc tgaaggcaga agactggggg |
| 196081 | aaatggggga catagagagc tgctcatgag gcgcagtcac cctgtgttct tccacttgac |
| 196141 | tgattggcac ccactctgtc ttgaagtgct ttggggacat tccttgccaa tcttggtgag |
| 196201 | tttagtcact gattccattc cactcagcac ccttgaccct tctgataact ccttcgacca |
| 196261 | cagtcYccct tcctttttta agaagggcca tatttactag agtttttttt tttttttttc |
| 196321 | ccaaactgtg tccaggttta ttaaagatac tttttgtgaa cagtcatggt atttcaggca |
| 196381 | ggacatgagc gacaatcatt aacagtatac aactttcaga ctccctcctt caatggactt |
| 196441 | ccaaaatcag aaagccatgg taaaacccaa tgaagtcttc atttggtacc ctgaagaggg |
| 196501 | aaagttcaga gtgagggttg acatttcaca ttcaacgtgc tgtttaacaa cttttcatga |
| 196561 | gctgaccctg actttcagga aatgaaatga aaatggcaga atttatctga agatccacaa |
| 196621 | tctagaaaca gagcacgtgt ctttcaaggg ttctcgctgg aaagtccaga ttgccagcct |
| 196681 | gactggtaac cagttattgg gggccagacc ccaacaggta tctgggtttg agggagttaa |
| 196741 | gtgtatactg aaggcagaga gggagagggg gaaataaaga ggaatttgtt tctccacacc |
| 196801 | acaaggcctt tgtgcgaagg tgactgtgtg tgtcaacgtc agacagtccc tctttctggg |
| 196861 | agccaagaag atgtctttaa aactagaagg gaaaggtgtt ttctccacat cagtccagct |
| 196921 | tcatagacat tctttttctt ttttttcttt cctgagacga agtcttgctc ttgttgccca |
| 196981 | ggttggagtg cagtggcacg atcttggctc actgcagcct ccgcctcccg gcttcaagtg |
| 197041 | attctcttgc ctcagcctcc tgagtagctg ggattacagg aacccatcat cacgcctggc |
| 197101 | tagtttttgt acttttagta gagacggggt ttcgccatgt tggtcgggct ggtctcaaac |
| 197161 | tcctgacctc aggtgatccg cctgcttcgg cctcccaaag tgctgggatt acaggcatga |
| 197221 | gccacggtgc ctggcctcag agacattcta ttagtgacat ataccccttc tccataaaac |
| 197281 | aacaatgacg tgttccgtgt gctaacaaca tggcttaaaa taaaggcata aaaacaattc |
| 197341 | tgcattttta taaaacttaa taaaaaaata atacttcaaa ctgcacagtc accagaaaKa |
| 197401 | cacagttatc aaaaatgcac acacttccct tggcatcccc agcgccttca actttctgtg |
| 197461 | cctgctctgt tttggcctct gcattttctg cagagttatt cccctccttg ccagcatggg |
| 197521 | ctttttcctt tttccccttt accttctctc ccttctttgc aggggcctct ttaggcttgg |
| 197581 | gcttcggagg agcaggtttg gcagacaacc ttgcagatct tctctgtggt ttgtccttac |
| 197641 | tttggtttta tctcctttag cgtccccttc agcctttctc ttgggcatgg tggaggcgac |
| 197701 | tgcagtggaa tgtaggtgct ggatctgggg atgcagcagt gcgtgggctt tggttggcct |
| 197761 | gggggatagt tcttacctct tcttcttcac actgctcata gagtatttct ttattagaaa |
| 197821 | tttaacattt aaaaaattca gtgtttttaa tgatgttgat tgtttttgtt ggtgctcaag |
| 197881 | ttaaaatctt tgttttcaac cctgtttttc cgataagcct tgttactttt agagtttgtt |
| 197941 | gttgcgaagc attagttttt tagcaataca tatgttcaat tacagaagaa attcccatga |
| 198001 | ttccttttcc taaggttgaa caatctggga aggagggatg ggaaggttaa tagcagcatc |
| 198061 | tatgggaggt ttttcccaag gatcagcaga cttaagcatt cttggctgca acttggccta |
| 198121 | aatcagtttt cccttggtga ctatcttcct aagtgctctt tattgtatat ttaggtgatt |
| 198181 | gtggttttgc atttctgtcc tctctctgat ctcttgctac agtctgaaca agtgtcggag |
| 198241 | gccgagttac tcccacagct gagcagagcc ccatcccagg ctgcagaaag tagtccagca |
| 198301 | aagaaggtaa ggtcagcctg aggatgctgt cagatggcgt tgcgagtccc accccgggct |
| 198361 | tcttatctct attagctgat ctattgtttt aaggtttctg tgtcctgaaa tgaatgtcaa |
| 198421 | tgatccagat ttcctagaat gtgggttctt tgtgaagatt ggggtcctga agattcctgg |
| 198481 | gcttgaccct tgatgcattt tgtacatgac agtacatact caacctcctt agccccccag |
| 198541 | ctaaacatgg tggccttgtt tatggcaaga gctaaaaggg ctgtggggag ggacagatgt |
| 198601 | ctttagcaaM accccatcca cctgctaagc ctctgtgaat acctcttggg gtggggtagt |
| 198661 | ctctcctttt tggtctttgt accatggttt cctcttgcat cgtggcattt tcacctggca |
| 198721 | tctgcccagc catgccattg ggtcacctgt aatttgctca gtaagtaccc ctcactcatc |
| 198781 | atgctctcca ttcatctcct caccctcttc ttgtccttct cccaggctct tctttcagag |
| 198841 | agagtgggcc Ytgggctcag gagcacaggt gaggtagagt cctccaaaga agcagaccaa |
| 198901 | ctgtgtcctt ccagaataca ctgtggctct ttttcttgac tttatgtagg aattggggat |
| 198961 | aggagaatga tgagcatatg actaagacct ttaaaaactt ccaaagctta ggtttttttt |
| 199021 | tgtttttgtt tttgtttttt tttcctgaga cRgagtctcg ctttgttgcc caggctggag |
| 199081 | tgcagtggcg cgatctcggc tcactgcaag ctcccaggtt cacgccattc tcctgcctca |
| 199141 | gccttccgag tagctgggac tacaggcatc ttccagcagg ccggctaatt ttttgtattt |
| 199201 | ttagtagaga cggggtttca ccgtgttagc caggatggtc tcgatctcct gacctcgtga |
| 199261 | tccgaccgtc tcggcctccc aaagtgctgg gattacaggc atgagccact gcgcccggcc |
| 199321 | aaaatttagg tttaaaaggc ttcaaagtga ggcaggagcc tatcttgtct tcagttagga |
| 199381 | aaggccccat ggtgtgagat gcacacaggg tctgtgtgag tttcaggtga caagaagtaa |
| 199441 | tagacttctt gaaacattta aaaagaaaaa gaaaaaaggg aagaccactg gactctgatg |
| 199501 | cctgtgtccc cccagttctt gctgagccat gggtcagtaa atagagtacc ctcctgatct |
| 199561 | tggggttggg gctaaaggca tgtgggacac tggaaggaag agtggaggga acattgaaat |
| 199621 | gtttaacttt ttaattttta ttttgtttta aatccacagg atgtactgta ttctcagcca |
| 199681 | ccatcaaagc ccattcgtag gaaattcaga ccagaaaacc aagctacaga aaaccaagag |
| 199741 | ccttccactg ctgcaagtgg gccagcttct gcggcaacca tgaaaccgca tccaacagtc |
| 199801 | caaaagtaag tagaccacat aaacaagagt gaggtaggaa tctcgctctg agatgtcgac |
| 199861 | ggatatgtaa atgtgttgac atttacgtgc gcaccaacag aaggatttca tggttgtaag |
| 199921 | tttatcttgc tcatctctgt gatgggattc aagtctccca gtcacataga aggctttata |
| 199981 | agctatagga cttaacctga cctaaagggg caggcgagtc ctaggctgtg gtgtttggtt |
| 200041 | tggtttgatt tctttccctt cctctcacct tctgttgttt cctactcctc ttccttgtga |
| 200101 | gggcccaaat tttcctggca agatgaaagt attgtaagtg gcaattttgc ctctctgcct |
| 200161 | gtgttatctc cttttccttc agggcaaatg gctagctcct tcacccacag acacactttc |
| 200221 | agtaggtgtg gtatgtgatt cttttaaaat ctccgtaaag agtttctgtt gttggtcgcc |
| 200281 | tcagcacaga atggatacat gcagaacttc cattatattt atctaaccag ctggaacagg |
| 200341 | tattggaggg agggtgaaga cacctgtgga catggccagg gactgagaca atgatgctct |
| 200401 | ggacaaacta tccttctctg taaaactttg gctttgagaa gagccatcac ttgcttcaat |
| 200461 | ttctttgtag tgaaattgag ggaggtgttg aggacagaaa cctgctcctg tcacttgact |
| 200521 | gcaagtgagc ctgaccggtc actaacataa atgctcaaga cccatcatga ccctgctttt |
| 200581 | ggggtcttgc aggtctcaga tgctgctgca gattttcttc tgcttacacg ttgtagaaYg |
| 200641 | aaaagcagca ggttctgtgg gagatgttag caacttgtct ttgaagccaa agtcccattc |
| 200701 | tgaatctctg cagccctctc cttcagaaga ttttcttttt ttcaaatgga caggtccctg |
| 200761 | gtggtatcag tccataaagg aagtatattt tgttcatcac attcaccgag aactttgacc |
| 200821 | atcttccagc ccctgttaag cctggaaggt gaagaatccc tatcagggac aaagatggct |
| 200881 | caattttctt ccatgttcta ccccagcact ccactttaat ttcagcttta tgttcatttt |
| 200941 | agtcagcagt ggcttaacag tctccagagt agatgtcagg gcatctggtg gagttcatac |
| 201001 | agagatcttt tctttttctc ttttttataa cagctttatt gcaatataat tcacatagca |
| 201061 | tacagttcat gtttttaaat gtacaattca gtggttctta gtttattcac aaagtcatgc |
| 201121 | aaccatcacc acaatctaac ttttgtcatg cctaaaataa tatatatcca cgcccattag |
| 201181 | caatcactcc ccattctctc cttccccctc agcccctggc aaccacgaat ctcctttctg |
| 201241 | tctccatggc tttgcatatt cttgtcattt cacataaatg gaataatgaa acgtggtctt |
| 201301 | ttgtgactgg cttctttcac ttagcataat attttcaagg tttatccaag ttgtagcata |
| 201361 | aatctgaact tatcttttta tggctgaata atattccatt gtatggatag accacatttt |
| 201421 | attcatccat tcatcagtca atggacattt tggttatttc tgcttttttg gctcttaaga |
| 201481 | gtaatgccac tatactgttt gtgtacatgt ttttgtgtgg acttacattt tcatttctct |
| 201541 | tgggtctgta cctaggagtg gaattgctag gtcatgtggt aactctgtct ttaatatttc |
| 201601 | gaagaactac cacacttttc atagtggctg caccatttta tgttcccgtt ggcaacgtat |
| 201661 | gagggttcca atttctccat tttgtcaaca cttatctttt ttttttatta tagccatcct |
| 201721 | agtgagtgtg aagtgtgatc tcatggtttt gatttgcatt tcttttgtgg ctaacgacat |
| 201781 | tgatcatctt ttcatgtgcc tattggccat ttgtttatct tctttggaga aatgtgtatt |
| 201841 | cacacccttt gcccactttt aattggatta ttaatctttt tacttttgag ttatgaattc |
| 201901 | tttatatatt ctggatacaa ctcccttgtt agatatatga gaaaattgag ccctctttgt |
| 201961 | aaatattttc tctcattctg tgggctgtct tttcactttc tattttttat ttttattttt |
| 202021 | tagagacaaa gtctcactct gtcacccagg ctggggtgca gtagcatgat catggctcat |
| 202081 | tgtagcctcg aattcctggg ctcaagtgat cctttccccc tcagcctcct gagtagctgg |
| 202141 | aactacaggc atgagccacc attcccagcc aatttcattt tattttttat agtgatggag |
| 202201 | tcttactatg ttgcccagtc tggtctcaaa ctcctggcct caagtgatca tcctgcctca |
| 202261 | acctctcaaa gtgctgggat tacaggcctg agccactgca cccagccaat tatcaatatt |
| 202321 | aatcagatct gtacagtggt cacagtccat tctagttcag ttgctctttt ctagaaccta |
| 202381 | tctcccagta atggcacata tggctgtttt tctaggagaa catcagttgc tcttttgctc |
| 202441 | taagaattcg gtgtttcttg ctgtgggatg aaaggttgta gattatccct gtggtaactg |
| 202501 | cactgattta tcatcccaac catggacggt tttgaaaagt aaagagagtg ctaatgtact |
| 202561 | tatgctggac agacagtttt aaatggagac tgtaatgggc gcagtaggat gcgtggtcac |
| 202621 | tctggttatg cgtgttctgt gaatttgttg ttaattggga aatactggta ttaaaacgtt |
| 202681 | ctgtattttt ctcaagagcc acagatatat gctggtccat gcacatacac gagtgcacat |
| 202741 | ttatgcacac acacacatat acttaggcat tcatgcctaa taatacataa ggtagaggta |
| 202801 | ggtagtctgg ctaagttaat gtagaatttt tgctaaaaat aatgttcttg tattattttc |
| 202861 | cttcagactt gttcacaggg gacacctgca ggtcttctaa gtgctattaa ttcttacaca |
| 202921 | aaaagggcat tccagttaat atggtttgtc aggaagaaat acttattaaa atgtcttgga |
| 202981 | ataaatggaa cacaaatgtt tgaaaaggcc catttcacag cttttattca agttataaaa |
| 203041 | tgggaattac ttcaggtgta aataattcat cccaataaat ttgtttcatt tggtgtctat |
| 203101 | cttcctgacc gcctgtgaat atgagatttt atttttggag ccctgtgttg ttaaccactt |
| 203161 | gttagtttaa cacctctcat gaatcagagt tctgaccctc aagggtccag acctctaagt |
| 203221 | cctttttcac tgaagtgttt gtgcttttct ataggcagtc ttccaaacag aagaaggcca |
| 203281 | ttcaaactgc tatccgcaaa aataaagagg caaacgcagt gctggctcgg ctgaacagtg |
| 203341 | agctccagca gcagctcaag gtaaggaatg agtcccagat ttggatgacc aggcttttcc |
| 203401 | ctggctttta aaaagctttt ttttaattta ataggcatat aataattgta catatttata |
| 203461 | gggtggggta catagtgatg tctccatacc tatcatgtac agtgatctga tgtgtccatt |
| 203521 | ggctgaccat ttagttccat ccctgttgat aagacttgcc tcttcgttct caaacccttt |
| 203581 | ttggaaggtg gagaattact gtatttaagt ttcatgggtt ggttcttgca tagtcagagg |
| 203641 | aagactagtg actttaatat gtaggcttca tattgtgtat gagttaaaag gaaagacaat |
| 203701 | ctattacgaa ggctgtttga agcattcttt attatccaaa gcttctgggt aagcccttaa |
| 203761 | ttatttacat cctctctgaa agaggatgta aatacttatt tcagtagaag gaagtcacct |
| 203821 | tggtactatc aaagcccagc ccctcctctg gtgccttgca aatggttcca tttcaccttc |
| 203881 | tcaatgacat cacccctgtt tctcttcatc gttactctcc cctgtgttag tttctcgggg |
| 203941 | ctgctataac aaaatactac aaatgtggtg gcttaagata accaaaaatg attctctcac |
| 204001 | agctctggag gctagaactc tgaaatcaag atattggcag agctatgatc tctttgaagg |
| 204061 | ctctagaaga tgatccttcc ttacctcttc ccagcttctg atgttgctgg cagccctggg |
| 204121 | cattccttgg cttgtagctg cgtcactcca atctctgcct ctgttgtcac atggccttct |
| 204181 | tcctgtatct ctctgtgtct gtgtccaaat ttctcttctt attgtatcct cttataagga |
| 204241 | caccaatcat tggattagga cccaccctaa tctagcatga cctcatctta acttgattac |
| 204301 | atctgcaaag aacctatttg catataaggt cacatttaca gatactgggg gttaggactt |
| 204361 | aaacatatat tttgagagga catgattcta cccactatat cccccagttc tctactggat |
| 204421 | cattcgccat ccacatgtaa atatgctcca gtattgtcca ttacacttgg ccaattactg |
| 204481 | cctcatttct ttggtcccct tcacagcaaa gtttcttagg agtggtttta ttcattatct |
| 204541 | ttactttcaa accagtaatt ttctcttcct tccacttcag ttggcttcgt cctcctccac |
| 204601 | tcacacccgt cttgtggcta aatccaatag ggctgtttta atattattat ttttttttca |
| 204661 | gaagaattct acacaattga tcatttcctc ctctgaaatg gcttttataa ctggtttttg |
| 204721 | acataccata ctctcctggt tttccttctg cttcactggt gcttcccatt ttcattctct |
| 204781 | gctgtctctt ccttccctga cttaactttt ataccccaac cccatttgcc ccaatatttt |
| 204841 | agtatgaaaa ttttcaaaca tacaggaaag ttgaaagaat cataagtgac tgcttgtata |
| 204901 | gacatcactt cagttctaca ataacatttt actatactta tttttaatca catatctacc |
| 204961 | catttatcct tgtgtccatc agttcatctt atttttgaca tattttcaaa taaattgcag |
| 205021 | atatctgtat actttccctg aatattttag catgcgtata attagctaga gttcaatata |
| 205081 | tttttacagt tttttcttat gatgtaaaat gtttatacaa aatgcatgca caaatattat |
| 205141 | tattattatt attattatta ttattattat tattattatt attatttgag atggagtctc |
| 205201 | gctctgtcgc ccacgctgga gtgcggtggc gtgatctctg ctcactgcaa gctccgcctc |
| 205261 | ctgggttcac accattcttc tggcttagcc tcctgagtag ctgggactac aggcgcctgc |
| 205321 | caccacgccc ggccaacttt ttgtattttt agtagaggtg gggtttcact gtgttagcca |
| 205381 | ggttggtctc gatttcctga ccttgtgatc tgcccgccta ggtctcccag agtgctggga |
| 205441 | ttacaggcgt gagccaccgc gccagcgcac gcacaaacct taagtgtaca tttgctgagt |
| 205501 | ttgtaacaag tacatgtact tacggaatcc aaacccccaa aaagatagag aatattacca |
| 205561 | tcaccccagg aagctccctc atgctttttc ctagtcaatt cctgccctac ccacacagag |
| 205621 | gcaatcacta ttctaatttt tataccatag ttttgcctgt tctataactt tatataagcg |
| 205681 | gtgtcttaca acttatactt cttgtaaagc ttctttcact aagtattatg tttttgagat |
| 205741 | tcatttatat tgttgtgtgt atcagtaatt cattcatttt tattgatgag tagtatccca |
| 205801 | ttgggtgaat atgttacagt ttggttatca attctattga cagacacctg agcggtttcc |
| 205861 | agtttttggc aatttggaat aaaactgcat tttttaataa gtctctgtat ggaaatatgt |
| 205921 | tctcatttct cttggattgg gattcgtagg tatcatggta agtgtatgtg attagtttct |
| 205981 | gagaaactgc tagatcattt tccaaggtgg ttgtagcaat ctgtactcct accactaatg |
| 206041 | tatgagagtt ccaattgttc cgcgtctttg ccaacatttg gtgttatgat tcttattaat |
| 206101 | tttagctagc cctgtagtga tatagttttt atcatctcct tgctgactaa ttatgttgtc |
| 206161 | cacttttctt tgtgattatt tacctttcat atcttacttt atgaaatatc ttttcaaatg |
| 206221 | ttatttttct ccaaagtagt ctatagattt attacaaaac caataaaaat cccagcagga |
| 206281 | ttttttgtga gtatagacaa gctaatccca aaacttatat gtaaaggcca aggagctgga |
| 206341 | attgcttaaa caaatttgaa aaggaagaac aaagttggag gaatcacttt actgtcaagc |
| 206401 | tccagtaatc aagatgctga tgcacaggtc agtggaatag gatacagagt cctgaaatag |
| 206461 | accacatgaa aacagccatt tgatctttga caaaggtgca aaatcgattc aatgagaaat |
| 206521 | aatagttttc aacaaattgt tttataacaa tttgtcattt gcatttaaaa aaataacctt |
| 206581 | gatctaagcc tcacatctat acagagatta acttgaaatg agtcatagat gtaaatgtaa |
| 206641 | aacatagagc tataaaattt ttagaagaaa acagaaaaat cttcatgatc tggagttaga |
| 206701 | aataacacca aaagcacaat ctataacaga gaaaaactaa aaaaattgga cttcatcaaa |
| 206761 | attaaaacct tttattttgc caagcacagt taagaaaatt aagagacaag ctgctgagtg |
| 206821 | ggagaaagta tttgcaaaac acgtatttga taaaagactt atatctcaat tatataaaga |
| 206881 | actcttaaaa ctcaataata agaactctca aaaccaaagc atctcaattt aaaaatgggc |
| 206941 | aaaggatttg aacagacact tcactaaaga atatataaag atggcaaata agtacatgag |
| 207001 | aagatattca acatcattag ccatttggga aatgcaaatt taaaccacaa ttacatacca |
| 207061 | ctatacacat actagaattg ctaaaacaaa aaatgtgaca tcaccaagtg ctggcaatta |
| 207121 | gaactctcat ttattggtgg tgggaatgca gaatggtacc accactctga aaaatcgttc |
| 207181 | agcagtttcc tatgaagtta tacatacact aaccatgtga cccagctgtc tctctacctt |
| 207241 | agtgaaatga aaatttatgt tcgctcaaaa atctatatat gaacttacat agcagtttta |
| 207301 | ttcatgatag cccaaacctg gaaacaacac aaatgtcctt caaaagatga atgaataaat |
| 207361 | tgggatacat tcatacaatg gaatagtact caacagagaa aagaaatgat acacaacttg |
| 207421 | gatgaaactc agacattatg cagagtgaga caagccagta tcaaaaggtt gtgtactgta |
| 207481 | tgattccttt tatttgacat tcttgaaaag gtaaaactat agtgatgtga aacaaatcag |
| 207541 | tggctatcag gggttatgca tgagatgaga gtttgtatga gtagtcccag ggaacttttg |
| 207601 | gggttggttg aactgtaatg tgtcttcttt gtggtggtgg ttacacaaat ctgtacacta |
| 207661 | aaagaaaaaa attgttttac cctatgataa ttttaaaaag ttatttttta aatttcaaag |
| 207721 | ttcattctgt tttctaaaac tttgtatgtg aacttgtttt gttttgcatt ttgtttggtt |
| 207781 | ttggttttct cctctctccc tctgctttgg ggtaggccag tttcatccat tgttctctac |
| 207841 | atttggtggg cccttttgga aactgatatt ccccagttct gaagggtttt ttattttcat |
| 207901 | ttttgttgtc gttgattatt tcctcccccc tgccctcacc cccatttttt tctcattctc |
| 207961 | ccttactgat attcagacat tggacttctt agcttagttc tctaattttc tcatattttc |
| 208021 | tcttctgttt tctttctttt ctcttcctac attttcttct ttatttttgt ctatttctgg |
| 208081 | aaacttaatt atatattcta aatcttctgt tgtttaataa ccaataattt tttgttctct |
| 208141 | gtttattttt gacggtatcc tattcttgtt tcattgttgt gatctttttt ttaattatac |
| 208201 | tttaagttct gggaaacatg tgcagaactc gttgtgatct ttttttatgg tcatttttct |
| 208261 | ttcttcttcc tgaatagttt gtgtttgttg tgttgtgttg tatgtgcttt atcttagatg |
| 208321 | ctttcctttg atatctgatg gcccttcttt gctcatattt aaaaggaaga tgaaaacact |
| 208381 | gattagaagt tcattgtacg ttgtaaggct tgtcaactct gagcttcact gtctggcaat |
| 208441 | ttggctcggc tgtatagttg gggaaccccc aaagggagta tgattagctc tttcctcttg |
| 208501 | gtctggttgg agtgtccaga gtggagactg ttttggtcac ctacatggag ggatagagtt |
| 208561 | ggttcagtgg agcaagaggg ctgtaggtct cagcattcca ggatattcat tcactctccc |
| 208621 | tatttctggc ccagtatctt gtcctcaact gtgcttgttt cttgctaatc cagatgctct |
| 208681 | ctgttgtacc ttttcaagaa ataaaccttc agcattctgc cctggtggca ggaatgatct |
| 208741 | ggggatctaa ttgcttctga aacagatttt cagccaacac ttattttagc caactcccat |
| 208801 | ctccccttat gtactcccac tttaataggt atctggtgtt gccaatttga gtctttcagg |
| 208861 | aattctccta cgtaaaatca agttggttct tatttttctt catgttagcc tgggactcag |
| 208921 | ctttctttac ttgatctgta tgagtggaaa agttctaagt ctagtgaaca gaagtctgaa |
| 208981 | gaatcattga aacagaacta tagtcatagc ccctcaatta attcccaggc taagccagtt |
| 209041 | tatagatcca gaacctcttg aatgatgaag gggcatctat gtctccttaa ggaaggaccc |
| 209101 | cactgtgcag ccaaaaattt acactgcaaa tctttctcct agcctttcct aagggggacc |
| 209161 | ctgctgccat ttaccagggt gactgtgcat tagaggaagg gaaataatta gacttttggg |
| 209221 | gaattaccag acactggctc tgaactcaca ctaattccag gagatgcaaa gtgtcactgt |
| 209281 | ggcccatcag agttggggct tagggagatc aggtgatcaa tggagtttta gcttaYgttc |
| 209341 | gtctcacagt ggacccagtg gactcccaaa ctcaccctRt ggttatttcc ctagctctgg |
| 209401 | aatggcatgc ttggaataga catactcagc agctggcaga attcccacat ttattttcct |
| 209461 | ggatttccta agtcatttac cattcaccag tttgccactc agctttcaaa ttttattgct |
| 209521 | atcatcttgt cccttttgtt ttccctgtct tgtgggtccg tgactttgaa aagttgctgt |
| 209581 | attgttttag tggtgctcca ggagggagag agtgtggtac ccctggtcag tcttccatct |
| 209641 | ttacctgaaa cctctatcta tactgttttg ctagatgatg ttatatggtc tcatggcttt |
| 209701 | gaagaccatc cataaactaa tgaaacctaa tttatgtctc aagccctact tctcctttcc |
| 209761 | tctgtgctct agacttgcat atataacttc ttactcaatg ttttctcctt agatatccaa |
| 209821 | tacacatctc agataattaa ttgccaaccc tctccttgaa atactcattt cttatctaat |
| 209881 | cttctcattc tcaatatcac taccagccag ctactttctt catacaaaaa tcccaggagt |
| 209941 | catcctcaga tttttcttct ctcctacatc aactttatca gcaagtccta ccagttctag |
| 210001 | attcaagatt Katctagaat tcatccactt ctcttcatct tcatggcttc taccctgacg |
| 210061 | gaagccattt tcatttctca cctgggtccc tgcagtaacc tcccagttgc tcccctgctt |
| 210121 | ccactctcat cctgctataa tctactgtct acacagtagc cagagtgaaa attttgaatt |
| 210181 | ttaagttggg tcatgttcct tccctgatta gaaacactca atgccaggcc gggtgcagtg |
| 210241 | gcccacgcct gtaatcccag cactttggga ggccgaggtg ggcagatcac gaggtcagga |
| 210301 | gatcgagacc atcctggcta acgtggtgaa accccgtttc tactaaaaat acagaaaatt |
| 210361 | agccgggcgt ggtggcgggY gcctgtagtc ccagctactY gggaggctga ggcaggagaa |
| 210421 | tggcgtgaac ctgggaggcg gagcttgcag tgagccgaga tcgcgccatt gcactccagc |
| 210481 | ctgggcgaca gagtgagact ctgtctcaga aaaaaaaaaa aaaagaaaga aagaaaccct |
| 210541 | caatgccatc cccSctacat ttccatacta tttcctctgc ccacaaggcc ctgtacgatc |
| 210601 | tggctccttg cttaaatctc tgacttcatt tcctgccaat gtcttccttg ttccatctac |
| 210661 | tctggtcaca caggcttttt gttcatcaaa ttcaccaaac tcactcgctc ctcagggcct |
| 210721 | ttgcagttgc ccttttgtct ctgtgaatat ccttcccccc atatcttcat atggctcact |
| 210781 | cctcatttca ttcaagtctt tgctcaagta ttgctaactc tgagaagcct tcccagggca |
| 210841 | ctctctctcc tttctcccat atccccctct gttactctct attatgttat cttatttcct |
| 210901 | tctggaagta tcttgttcat taactttatt atctttctca agtcatttga atataagccc |
| 210961 | tattgctctg atctggcata ttctttgctg ataccatcac agctacagca ttacctcacc |
| 211021 | tgtaggagat agaaaccaag agacttccgg ctgggtggct aggagagcat gtgaataaat |
| 211081 | acttgtgaag ttgtatactg actattagta tgctgattct tctgaatctc acaattcagc |
| 211141 | ctttctacag gattctttcc caccttgact gattctactt gaataccttt atgtgtcccc |
| 211201 | tctcttcctg ccaaaaaaat ccgttttagg cccgaaagtt tgaattccca aagagcagat |
| 211261 | tgdtcagcct dttttctgcc tttcacccag atgttgtacc ttacaccata tgttgtgcag |
| 211321 | aaacttggat gagactttgg tggaggggca actagcgtac ctttttggcc ctcagaccta |
| 211381 | actgagtatg gtgcgggatt acgagccaga gattgcaact agaaatttaa gtcctgttgg |
| 211441 | ctcactgctc agtattggga gtctgttttt gctttctgct ttctcctagt cttttaattt |
| 211501 | ttaatgactc tgtctaccag ctactgatct tgtcaaaaca ggctaaaact agggcagggt |
| 211561 | ggtagagggg agtaggtcag tttcatttca tcacgttcag aaaggagttg agagattaca |
| 211621 | aattgtgaag ccccttttgt ctggtcacca gcattaaatc tgatacctcc cagtcctcct |
| 211681 | gttcttgagt tatacagcac ttgtatttgt gggtccagtt ataagtttcc ttttaaatct |
| 211741 | gatgaagtag ggatgaattc aagaaatgac ccctttgctt ttttgttttt cttgtgtttt |
| 211801 | gtacaactca ggaggttcat caagaacgaa ttgcattgga aaaccaattg gaacaacttc |
| 211861 | gtccggtcac tgtgttgtga cccccccatg gttcaagtga cagtgggtga ccttgtctgc |
| 211921 | caagatcttt cttttgaatg ttttgaaccc aactacttgt catagatgtt tgactgtgtc |
| 211981 | aaaagctgtg agcagcaaaa tataatccat atgacctttt ctcttgcact tcacttgtat |
| 212041 | gttttactgt ggtggaaaaa atacttcaac tgattatcac acttgaaatg ctaattatca |
| 212101 | gtggaatttg tctcctattc ttaagtactt ctgcaaggta ttagatgctg ctccttacca |
| 212161 | aaaatcagtc ttctagcaaa taaaaataac ttagagcatt atttatttaa agaatttatg |
| 212221 | ttttctataa aaccttataa ccaagaacct tcaggatgct tgtaaccaag tactttcagg |
| 212281 | atgctttttg tatgtatatc acatagagta ggttggtttc ctgagtaatt gggattccaa |
| 212341 | ctagatagtc attggtgtga ccataataaa aacaaaagct aaaataaaag catcagattt |
| 212401 | agactggcgc tgtgccctct accactatat gccaaaaatt gcttctctag tgccattttg |
| 212461 | atattatatc tagctataaa taatacatga ctatcgtttt ctattgaatg tcaaagactt |
| 212521 | actgggtctt tacacctctg taaagtggag gttttggcaa tgagctgttt aacttggcca |
| 212581 | agctaggctg tcaacccaga caactcagtc ctttcatgta aatttttggg caagagaagt |
| 212641 | ctgctttgac tgccttgcct gaccaaatta aataaatagt tgccagatct ctacttttat |
| 212701 | cctgcatggg ataatatatc tgtaaatgca tgagtttgga aaccacccat caaatatgaa |
| 212761 | aggctgatga gactgttagg attgataaac atttgttgtt tgaacccagc tatccaaact |
| 212821 | ggggaagaag gggaaatggt tgttaccaac aatctctagt agttatttta atctttattt |
| 212881 | taacctggat tgcaaatgta tggggtatga ggagataatg aaagaaaagt ggtcctcatg |
| 212941 | ctggatatgt gactgttttg ttctctgtaa agtgtattct tgggaagtga ttggtttata |
| 213001 | tcagattcag aggagcccaa ttaactccag tgtaggtaat gtaaaacagt aagctcaaat |
| 213061 | tgcagaagcc Rgtatcctac agaaatttaa gtagctactg cttctcctga aagcccaagt |
| 213121 | tgaaaatggg accacagggg cacagggcta ctgaccctca cagggcatgc cagcgttaac |
| 213181 | accactttga gagaccaatg gcaagaaatg ctgtctcttg tgcattttac taatttcccc |
| 213241 | attcttaggg tagcagggtg ggggtgtata gggtcttttt gaagcagttt tggataggtt |

Following are NRP1, NID2, ENDO180, CDK10, FPGT, CARK, PCLO and REPS2 complementary DNA sequences (cDNAs; SEQ ID NO: 8, 9, 10, 11, 12, 13, 14, 15, 16 and 17, respectively). Following is a NRP1 cDNA sequence (SEQ ID NO: 8).

>gi|4505456|ref|NM_003873.1− Homo sapiens neuropilin 1 (NRP1), mRNA
ATGGAGAGGGGGCTGCCGCTCCTCTGCGCCGTGCTCGCCCTCGTCCTCGCCGCGGCCGGCGCTTTTCGCAACCATGAATGTGGCGA
TACTATAAAAATTGAAAGCCCCGGGTACCTTACATCTCCTGGTTATCCTCATTCTTATCACCCAAGTGAAAAATGCGAATGGCTGA
TTCAGGCTCCGGACCCATACCAGAGAATTATGATCAACTTCAACCCTCACTTCGATTTGGAGGACACAGACTGCAAGTATGACTAC
GTGGAAGTCTTCGATGGAGAAAATGAAAATGGACATTTTAGGGGAAAGTTCTGTGGAAAGATAGCCCCTCCTCCTGTTGTGTCTTC
AGGGCCATTTCTTTTTATCAAATTTGTCTCTGACTACGAAACACATGGTGCAGGATTTTCCATACGTTATGAAATTTTCAAGAGAG
GTCCTGAATGTTCCCAGAACTACACAACACCTAGTGGAGTGATAAAGTCCCCCGGATTCCCTGAAAAATATCCCAACAGCCTTGAA
TGCACTTATATTGTCTTTGCGCCAAAGATGTCAGAGATTATCCTGGAATTTGAAAGCTTTGACCTGGAGCCTGACTCAAATCCTCC
AGGGGGGATGTTCTGTCGCTACGACCGGCTAGAAATCTGGGATGGATTCCCTGATGTTGGCCCTCACATTGGGCGTTACTGTGGAC
AGAAAACACCAGGTCGAATCCGATCCTCATCGGGCATTCTCTCCATGGTTTTTTACACCGACAGCGCGATAGCAAAAGAAGGTTTC
TCAGCAAACTACAGTGTCTTGCAGAGCAGTGTCTCAGAAGATTTCAAATGTATGGAAGCTCTGGGCATGGAATCAGGAGAAATTCA
TTCTGACCAGATCACAGCTTCTTCCCAGTATAGCACCAACTGGTCTGCAGAGCGCTCCCGCCTGAACTACCCTGAGAATGGGTGGA
CTCCCGGAGAGGATTCCTACCGAGAGTGGATACAGGTAGACTTGGGCCTTCTGCGCTTTGTCACGGCTGTCGGGACACAGGGCGCC
ATTTCAAAAGAAACCAAGAAGAAATATTATGTCAAGACTTACAAGATCGACGTTAGCTCCAACGGGGAAGACTGGATCACCATAAA
AGAAGGAAACAAACCTGTTCTCTTTCAGGGAAACACCAACCCCACAGATGTTGTGGTTGCAGTATTCCCCAAACCACTGATAACTC
GATTTGTCCGAATCAAGCCTGCAACTTGGGAAACTGGCATATCTATGAGATTTGAAGTATACGGTTGCAAGATAACAGATTATCCT
TGCTCTGGAATGTTGGGTATGGTGTCTGGACTTATTTCTGACTCCCAGATCACATCATCCAACCAAGGAGACAGAAACTGGATGCC
TGAAAACATCCGCCTGGTAACCAGTCGCTCTGGCTGGGCACTTCCACCCGCACCTCATTCCTACATCAATGAGTGGCTCCAAATAG
ACCTGGGGGAGGAGAAGATCGTGAGGGGCATCATcATTCAGGGTGGGAAGCACCGAGAGAACAAGGTGTTCATGAGGAAGTTCAAG
ATCGGGTACAGCAACAACGGCTCGGACTGGAAGATGATCATGGATGACAGCAAACGCAAGGCGAAGTCTTTTGAGGGCAACAACAA
CTATGATACACCTGAGCTGCGGACTTTTCCAGCTCTCTCCACGCGATTCATCAGGATCTACCCCGAGAGAGCCACTCATGGCGGAC
TGGGGCTCAGAATGGAGCTGCTGGGCTGTGAAGTGGAAGCCCCTACAGCTGGACCGACCACTCCCAACGGGAACTTGGTGGATGAA
TGTGATGACGACCAGGCCAACTGCCACAGTGGAACAGGTGATGACTTCCAGCTCACAGGTGGCACCACTGTGCTGGCCACAGAAAA
GCCCACGGTCATAGACAGCACCATACAATCAGAGTTTCCAACATATGGTTTTAACTGTGAATTTGGCTGGGGCTCTCACAAGACCT
TCTGCCACTGGGAACATGACAATCACGTGCAGCTCAAGTGGAGTGTGTTGACCAGCAAGACGGGACCCATTCAGGATCACACAGGA
GATGGCAACTTCATCTATTCCCAAGCTGACGAAAATCAGAAGGGCAAAGTGGCTCGCCTGGTGAGCCCTGTGGTTTATTCCCAGAA
CTCTGCCCACTGCATGACCTTCTGGTATCACATGTCTGGGTCCCACGTCGGCACACTCAGGGTCAAACTGCGCTACCAGAAGCCAG
AGGAGTACGATCAGCTGGTCTGGATGGCCATTGGACACCAAGGTGACCACTGGAAGGAAGGGCGTGTCTTGCTCCACAAGTCTCTG
AAACTTTATCAGGTGATTTTCGAGGGCGAAATCGGAAAAGGAAACCTTGGTGGGATTGCTGTGGATGACATTAGTATTAATAACCA
CATTTCACAAGAAGATTGTGCAAAACCAGCAGACCTGGATAAAAAGAACCCAGAAATTAAAATTGATGAAACAGGGAGCACGCCAG
GATACGAAGGTGAAGGAGAAGGTGACAAGAACATCTCCAGGAAGCCAGGCAATGTGTTGAAGACCTTAGAACCCATCCTCATCACC
ATCATAGCCATGAGCGCCCTGGGGGTCCTCCTGGGGGCTGTCTGTGGGGTCGTGCTGTACTGTGCCTGTTGGCATAATGGGATGTC
AGAAAGAAACTTGTCTGCCCTGGAGAACTATAACTTTGAACTTGTGGATGGTGTGAAGTTGAAAAAAGACAAACTGAATACACAGA
GTACTTATTCGGAGGCATGA

Following is a NID2 cDNA sequence (SEQ ID NO: 9).

>gi|33469988|ref|NM_007361.2| Homo sapiens nidogen 2 (osteonidogen) (NID2), mRNA
GCCTTAGAAAAGTTAACGAGAACCAGATGTGGTGGCCACTGCCGAACTTTCTCAGAGCCGGTGATTGGTCCCCAGCCGAGGGCCTC
AGCCAATTAGCTTGCTGGGTGGGCCTGGAGTCCCGCCCCGCCCAGGCGCCCGCGGAGATCCAGGTTCGAGGCTGGCGGGGCGCGGA
GAGTGGGCTGGAGGCCGGGGCGGGACGCGTTGTGCAGCGGGTAAGCGCACGGCCGAGCGAGCATGGAGGGGGACCGGGTGGCCGGG
CGGCCGGTGCTGTCGTCGTTACCAGTGCTACTGCTGCTGCAGTTGCTAATGTTGCGGGCCGCGGCGCTGCACCCAGACGAGCTCTT
CCCACACGGGGAGTCGTGGGGGGACCAGCTCCTCCAGGAAGGCGACGACGAAAGCTCAGCCGTGGTGAAGCTGGCGAATCCCCTGC
ACTTCTACGAAGCCCGATTCAGCAACCTCTACGTGGGCACCAACGGCATCATCTCCACTCAGGACTTCCCCAGGGAAACGCAGTAT
GTGGACTATGATTTCCCCACCGACTTCCCGGCCATCGCCCCTTTTCTGGCGGACATCGACACGAGCCACGGCAGAGGCCGAGTCCT
GTACCGAGAGGACACCTCCCCCGCAGTGCTGGGCCTGGCCGCCCGCTATCTGCGCGCTGGCTTCCCGCGCTCTGCGCGCTTTACCC
CCACCCACGCCTTCCTGGCCACCTGGGAGCAGGTAGGCCCTTACGAGGAGGTCAAGCGCGGGGCGCTGCCCTCGGGAGAGCTGAAC
ACTTTCCAGGCAGTTTTGGCATCTGATGGGTCTGATAGCTACGCCCTCTTTCTTTATCCTGCCAACGGCCTGCAGTTCCTTGGAAC
CCGCCCCAAAGAGTCTTACAATGTCCAGCTTCAGCTTCCAGCTCGGGTGGGCTTCTGCCGAGGGGAGGCTGATGATCTGAAGTCAG
AACGACCATATTTCAGCTTGACTAGCACTGAACAGTCTCTGAAAAATCTCTATCAACTAAGCAACCTGGGGATCCCTGGAGTCTGG
GCTTTCCATATCGGCAGCACTTCCCCGTTGGACAATGTCAGGCCAGCTGCAGTTGGAGACCTTTCCGCTGCCCACTCTTCTGTTCC
CCTGGGACGTTCCTTCAGCCATGCTACAGCCCTGGAAAGTGACTATAATGAGGACAATTTGGATTACTACGATGTGAATGAGGAGG
AAGCTGAATACCTTCCGGGTGAACCAGAGGAGGCATTGAATCGCCACAGCAGCATTGATGTTTCCTTCCAATCCAAAGTGGATACA
AAGCCTTTAGAGGAATCTTCCACCTTGGATCCTCACACCAAAGAAGGAACATCTCTGGGAGAGGTAGGGGGCCCAGATTTAAAAGG
CCAAGTTGAGCCCTGGGATGAGAGAGAGACCAGAAGCCCAGCTCCACCAGAGGTAGACAGAGATTCACTGGCTCCTTCCTGGGAAA
CCCCACCACCGTACCCCGAAAACGGAAGCATCCAGCCCTACCCAGATGGAGGGCCAGTGCCTTCGGAAATGGATGTTCCCCCAGCT
CATCCTGAAGAAGAAATTGTTCTTCGAAGTTACCCTGCTTCAGGTCACACTACACCCTTAAGTCGAGGGACGTATGAGGTGGGACT
GGAAGACAACATAGGTTCCAACACCGAGGTCTTCACGTATAATGCTGCCAACAAGGAAACCTGTGAACACAACCACAGACAATGCT
CCCGGCATCCCTTCTGCACGGACTATGCCACTGGCTTCTGCTGCCACTGCCAATCCAAGTTTTATGGAAATCGGAAGCACTGTCTG
CCTGAGGGGGCACCTCACCGAGTGAATGGGAAAGTGAGTGGCCACCTCCACGTGGGCCATACACCCGTGCACTTCACTGATGTGGA
CCTGCATGCGTATATCGTGGGCAATGATGGCAGAGCCTACACGGCCATCAGCCACATCCCACAGCCAGCAGCCGAGGCCCTCCTCC
CCCTCACACCAATTGGAGGCCTGTTTGGCTGGCTCTTTGCTTTAGAAAAACCTGGCTCTGAGAACGGCTTCAGCCTCGCAGGTGCT
GCCTTTACCCATGACATGGAAGTTACATTCTACCCGGGAGAGGAGACGGTTCGTATCACTCAAACTGCTGAGGGACTTGACCCAGA
GAACTACCTGAGCATTAAGACCAACATTCAAGGCCAGGTGCCTTACGTCCCAGCAAATTTCACAGCCCACATCTCTCCCTACAAGG
AGCTGTACCACTACTCCGACTCCACTGTGACCTCTACAAGTTCCAGAGACTACTCTCTGACTTTTGGTGCAATCAACCAAACATGG
TCCTACCGCATCCACCAGAACATCACTTACCAGCTGTGCAGGCACGCCCCCAGACACCCGTCCTTCCCCACCACCCAGCAGCTGAA
CGTGGACCGGGTCTTTGCCTTGTATAATGACGAAGAAAGAGTGCTTAGATTTGCTGTGACCAATCAAATTGGCCCGGTCAAAGAAG
ATTCAGACCCCACTCCGGGGAATCCTTGCTATGATGGGAGCCACATGTGTGACACAACAGCACGGTGCCATCCAGGGACAGGTGTA
GATTACACCTGTGAGTGCGCATCTGGGTACCAGGGAGATGGACGGAACTGTGTGGATGAAAATGAATGTGCAACTGGCTTTCATCG
CTGTGGCCCCAACTCTGTATGTATCAACTTGCCTGGAAGCTACAGGTGTGAGTGCCGGAGTGGTTATGAGTTTGCAGATGACCGGC
ATACTTGCATCTTGATCACCCCACCTGCCAACCCCTGTGAGGATGGCAGTCATACCTGTGCTCCTGCTGGGCAGGCCCGGTGTGTT
CACCATGGAGGCAGCACGTTCAGCTGTGCCTGCCTGCCTGGTTATGCCGGCGATGGGCACCAGTGCACTGATGTAGATGAATGCTC
AGAAAACAGATGTCACCCTGCAGCTACCTGCTACAATACTCCTGGTTCCTTCTCCTGCCGTTGTCAACCCGGATATTATGGGGATG
GATTTCAGTGCATACCTGACTCCACCTCAAGCCTGACACCCTGTGAACAACAGCAGCGCCATGCCCAGGCCCAGTATGCCTACCCT
GGGGCCCGGTTCCACATCCCCCAATGCGACGAGCAGGGCAACTTCCTGCCCCTACAGTGTCATGGCAGCACTGGTTTCTGCTGGTG
CGTGGACCCTGATGGTCATGAAGTTCCTGGTACCCAGACTCCACCTGGCTCCACCCCGCCTCACTGTGGACCATCACCAGAGCCCA
CCCAGAGGCCCCCGACCATCTGTGAGCGCTGGAGGGAAAACCTGCTGGAGCACTACGGTGGCACCCCCCGGGATGACCAGTACGTG
CCCCAGTGCGATGACCTGGGCCACTTCATCCCCCTGCAGTGCCACGGAAAGAGCGACTTCTGCTGGTGTGTGGACAAAGATGGCAG
AGAGGTGCAGGGCACCCGCTCCCAGCCAGGCACCACCCCTGCGTGTATACCCACCGTCGCTCCACCCATGGTCCGGCCCACGCCCC
GGCCAGATGTGACCCCTCCATCTGTGGGCACCTTCCTGCTCTATACTCAGGGCCAGCAGATTGGCTACTTACCCCTCAATGGCACC
AGGCTTCAGAAGGATGCAGCTAAGACCCTGCTGTCTCTGCATGGCTCCATAATCGTGGGAATTGATTACGACTGCCGGGAGAGGAT
GGTGTACTGGACAGATGTTGCTGGACGGACAATCAGCCGTGCTGGTCTGGAACTGGGAGCAGAGCCTGAGACGATCGTGAATTCAG
GTCTGATAAGCCCTGAAGGACTTGCCATAGACCACATCCGCAGAACAATGTACTGGACGGACAGTGTCCTGGATAAGATAGAGAGC
GCCCTGCTGGATGGCTCTGAGCGCAAGGTCCTCTTCTACACAGATCTGGTGAATCCCCGTGCCATCGCTGTGGATCCAATCCGAGG
CAACTTGTACTGGACAGACTGGAATAGAGAAGCTCCTAAAATTGAAACGTCATCTTTAGATGGAGAAAACAGAAGAATTCTGATCA
ATACAGACATTGGATTGCCCAATGGCTTAACCTTTGACCCTTTCTCTAAACTGCTCTGCTGGGCAGATGCAGGAACCAAAAAACTG
GAGTGTACACTACCTGATGGAACTGGACGGCGTGTCATTCAAAACAACCTCAAGTACCCCTTCAGCATCGTAAGCTATGCAGATCA
CTTCTACCACACAGACTGGAGGAGGGATGGTGTTGTATCAGTAAATAAACATAGTGGCCAGTTTACTGATGAGTATCTCCCAGAAC
AACGATCTCACCTCTACGGGATAACTGCAGTCTACCCCTACTGCCCAACAGGAAGAAAGTAAGTACAGTAATGTAAAGGAAGACTT
GGAGTTTACAATCAGAACCTGGACCCTAAAGAACAGTGACTGCAAAGGCAAAGAAAGTAAAAAAGGAATTGGCCATTAGACGTTCC
TGAGCATCCAAGATGAACATTTTGTAGTGCAAAAAGACTTTTGTGAAAAGCTGATACCTCAATCTTTACTACTGTATTTTTAAAAA
TGAAGGTTGTTATTGCAAGTTTAAAAAGGTAACAGAATTTTAACTGTTGCTTATTAAAGCAACTTCTTGTAAACATTTATCATTAA
TATTTAAAAGATCAAATTCATTCAACTAAGAATTAGAGTTTAAGACTCTAAACCTGATTTTTGCCATGGATTCCTTCTGGCCAAGA
AATTAAAGCACATGTGATCAATATAACAATATAATCCTAAACCTTGACAGTTGGAGAAGCCAATGCAGAACTGATGGGAAAGGACC
AATTATTTATAGTTTCCCAACAAAAGTTCTAAGATTTTTTACCTCTGCATCAGTGCATTTCTATTTATATCAAAAGGTGCTAAAAT
GATTCAATTTGCATTTTCTGATCCTGTAGTGCCTCTATAGAAGTACCCACAGAAAGTAAAGTATCACATTTATAAATACCAAAGAT
GTAACAATTTTAAAATTTTCTAGATTACTCCAATAAAGTGTTTTAAGTTTTCCTATGAAAAAAAAAAAAAAAAAAAA

Following is a ENDO180 cDNA sequence (SEQ ID NO: 10).

>gi|5174484|ref|NM_006039.1| Homo sapiens mannose receptor, C type 2 (MRC2), mRNA
CGGAGGAGGACGCGAGCCCCTTGCGGGCGGTCATCACAGCCCAGCCTCGGGGCTGCCACAGCGCGTTGCGCCTGTGCGCCCTCGGT
CCCCGCGTCCACTGAGCGCCGCGCTCGGGGATGGGGCCCGGCCGGCCGGCCCCCGCGCCCTGGCCTCGTCACCTGCTGCGCTGCGT
CCTGCTCCTCGGGTGCCTGCACCTCGGCCGTCCCGGCGCCCCTGGGGACGCCGCCCTCCCGGAACCCAACGTCTTCCTCATCTTCA
GCCATGGACTGCAGGGCTGCCTGGAGGCCCAGGGCGGGCAGGTCAGAGTCACCCCGGCTTGCAATACCAGCCTCCCTGCCCAGCGC
TGGAAGTGGGTCTCCCGAAACCGGCTATTCAACCTGGGTACCATGCAGTGCCTGGGCACAGCCTGGCCAGGCACCAACACCACGGC
CTCCCTGGGCATCTATGAGTGTGACCGGGAAGCACTGAATCTTCGCTGGCATTGTCGTACACTGGGTGACCAGCTGTCCTTGCTCC
TGGGGGCCCGCACCAGCAACATATCCAACCCTGGCACCCTTGAGCGTGGTGACCAGACCCGCAGTGGCCAGTGGCGCATCTACGGC
AGCGAGGAGGACCTATGTGCTCTGCCCTACCACGAGGTCTACACCATCCAGGGAAACTCCCACGGAAAGCCGTCCACCATCCCCTT
CAAATATGACAACCAGTGGTTCCACGGCTGCACCAGCACGGGCCGCGAGGATGGTCACCTGTGGTGTCCCACCACCCAGGACTACG
GCAAAGACGAGCGCTGGGGCTTCTGCCCCATCAAGAGTAACGACTGCGAGACCTTCTGGGACAAGGACCAGCTGACTGACAGCTGC
TACCAGTTTAACTTCCAGTCCACGCTGTCGTGGAGGGAGCCCTGGGCCAGCTGCGAGCAGCAGGGTGCCGATCTCCTGAGCATCAC
GGAGATCCACGAGCACACCTACATCAACGGCCTCCTCACTCGGTACAGCTCCACCCTGTGGATCGGCTTGAATCACTTGGACACGA
CCGGAGGCTGGCAGTGGTCGGACAACTCGCCCCTCAAGTACCTCAACTGGGAGAGTGACCAGCCGGACAACCCCAGTGAGGAGAAC
TGTGGAGTGATCCGCACTGAGTCCTCGGGCGGCTGGCAGAACCGTGACTGCAGCATCGCGCTGCCCTATGTGTGCAAGAAGAAGCC
CAACGCCACGGCCGAGCCCACCCCTCCAGACAGGTGGGCCAATGTGAAGGTGGAGTGCGAGCCGAGCTCCCAGCCCTTCCAGGGCC
ACTGCTACCGCCTGCAGGCCGACAAGCGCAGCTGGCAGGAGTCCAAGAAGGCATGTCTACGGGGCGGTGGCGACCTGGTCAGCATC
CACAGCATGGCGGAGCTGGAATTCATCACCAAGCAGATCAAGCAAGAGGTCGAGGAGCTGTGGATCGGCCTCAACGATTTGAAACT
GCAGATGAATTTTGAGTGGTCTGACGGGAGCCTTGTGACCTTCACCCACTGGCACCCCTTTGAGCCCAACAACTTCCGGGACAGTC
TGGAGGACTGTGTCACCATCTGGGGCCCGGAAGGCCGCTGGAACGACAGTCCCTGTAACCAGTCCTTGCCATCCATCTGCAAGAAG
GCAGGCCAGCTGAGCCAGGGGGCCGCCGAGGAGGACCATGGCTGCCGGAAGGGTTGGACGTGGCACAGCCCATCCTGCTACTGGCT
GGGAGAAGACCAAGTGACCTACAGTGAGGCCCGGCGCCTGTGCACTGACCATGGCTCTCAGCTGGTCACCATCACCAACAGGTTCG
AGCAGGCCTTCGTCAGCAGCCTCATCTACAACTGGGAGGGCGAGTACTTCTGGACGGCCCTGCAGGACCTCAACAGCACCGGCTCC
TTCTTCTGGCTCAGTGGGGATGAAGTCATGTACACCCACTGGAACCGGGACCAGCCCGGGTACAGCCGTGGGGGCTGCGTGGCGCT
GGCCACTGGCAGCGCCATGGGGCTGTGGGAGGTGAAGAACTGTACCTCGTTCCGGGCCCGCTACATCTGCCGGCAGAGCCTGGGCA
CTCCAGTGACGCCGGAGCTGCCCGGGCCAGATCCCACGCCCAGCCTCACTGGCTCCTGTCCCCAGGGCTGGGCCTCGGACACCAAA
CTCCGGTATTGCTATAAGGTGTTCAGCTCAGAGCGGCTGCAGGACAAGAAGAGCTGGGTCCAGGCCCAGGGGGCCTGCCAGGAGCT
GGGGGCCCAGCTGCTGAGCCTCCCCAGCTACGAGGAGGAGCACTTTGTGGCCAACATGCTCAACAAGATCTTCGGTGAATCAGAAC
CCGAGATCCACGAGCAGCACTGGTTCTGGATCGGCCTGAACCGTCGGGATCCCAGAGGGGGTCAGAGTTGGCGCTGGAGCGACGGC
GTAGGCTTCTCTTACCACAATTTCGACCGGAGCCGGCACCACGACGACGACATCCGAGGCTGTGCGGTGCTGGACCTGGCCTCCCT
GCACTGGGTGGCCATGCAGTGCGACACACAGCTGGACTGGATCTGCAAGATCCCCAGAGGTACGGACGTGCGGGAGCCCGACGACA
GCCCTCAAGGCCGACGGGAATGGCTGCGCTTCCAGGAGGCCGAGTACAAGTTCTTTGAGCACCACTCCACGTGGGCGCAGGCGCAG
CGCATCTGCACGTGGTTCCAGGCCGAGCTGACCTCCGTGCACAGCCAGGCAGAGCTAGACTTCCTGAGCCACAACTTGCAGAAGTT
CTCCCGGGCCCAGGAGCAGCACTGGTGGATCGGCCTGCACACCTCTGAGAGCGATGGGCGCTTCAGATGGACAGATGGTTCCATTA
TAAACTTCATCTCCTGGGCACCAGGCAAACCTCGGCCTGTCGGCAAGGACAAGAAGTGCGTGTACATGACAGCCAGCCGAGAGGAC
TGGGGGGACCAGAGGTGCCTGACAGCCTTGCCCTACATCTGCAAGCGCAGCAACGTCACCAAAGAAACGCAGCCCCCAGACCTGCC
AACTACAGCCCTGGGGGGCTGCCCCTCTGACTGGATCCAGTTCCTCAACAAGTGTTTTCAGGTCCAGGGCCAGGAACCCCAGAGCC
GGGTGAAGTGGTCAGAGGCACAGTTCTCCTGTGAACAGCAAGAGGCCCAGCTGGTCACCATCACAAACCCCTTAGAGCAAGCATTC
ATCACAGCCAGCCTGCCCAATGTGACCTTTGACCTTTGGATTGGCCTCCATGCCTCGCAGAGGGACTTCCAGTGGGTGGAGCAGGA
GCCTTTGATGTATGCCAACTGGGCACCTGGGGAGCCCTCTGGCCCTAGCCCTGCTCCCAGTGGCAACAAACCGACCAGCTGTGCGG
TGGTCCTGCACAGCCCCTCAGCCCACTTCACTGGCCGCTGGGACGATCGGAGCTGCACGGAGGAGACCCATGGCTTCATCTGCCAG
AAGGGCACGGACCCCTCCCTGAGCCCGTCCCCAGCAGCGCTGCCCCCCGCCCCGGGCACTGAGCTCTCCTACCTCAACGGCACCTT
CCGGCTGCTTCAGAAGCCGCTGCGCTGGCACGATGCCCTCCTGCTGTGTGAGAGCCACAATGCCAGCCTGGCCTACGTGCCCGACC
CCTACACCCAGGCCTTCCTCACGCAGGCTGCCCGAGGGCTGCGCACGCCGCTCTGGATTGGGCTGGCTGGCGAGGAGGGCTCTCGG
CGGTACTCCTGGGTCTCAGAGGAGCCGCTGAACTACGTGGGCTGGCAGGACGGGGAGCCGCAGCAGCCGGGGGGCTGTACCTACGT
AGATGTGGACGGGGCCTGGCGCACCACCAGCTGTGACACCAAGCTGCAGGGGGCTGTGTGTGGGGTTAGCAGTGGGCCCCCTCCTC
CCCGAAGAATAAGCTACCATGGCAGCTGTCCCCAGGGACTGGCAGACTCCGCGTGGATTCCCTTCCGGGAGCACTGCTATTCTTTC
CACATGGAGCTGCTGCTGGGCCACAAGGAGGCGCGACAGCGCTGCCAGAGAGCGGGTGGGGCCGTCCTGTCTATCCTGGATGAGAT
GGAGAATGTGTTTGTCTGGGAGCACCTGCAGAGCTATGAGGGCCAGAGTCGGGGCGCCTGGCTGGGCATGAACTTCAACCCCAAAG
GAGGCACTCTGGTCTGGCAGGACAACACAGCTGTGAACTACTCCAACTGGGGGCCCCCGGGCTTGGGCCCCAGCATGCTGAGCCAC
AACAGCTGCTACTGGATTCAGAGCAACAGCGGGCTATGGCGCCCCGGCGCTTGCACCAACATCACCATGGGTGTCGTCTGCAAGCT
TCCTCGTGCTGAGCAGAGCAGCTTCTCCCCATCAGCGCTTCCAGAGAACCCAGCGGCCCTGGTGGTGGTGCTGATGGCGGTGCTGC
TGCTCCTGGCCTTGCTGACCGCAGCCCTCATCCTTTACCGGAGGCGCCAGAGCATCGAGCGCGGGGCCTTTGAGGGTGCCCGCTAC
AGCCGCAGCAGCTCCAGCCCCACCGAGGCCACTGAGAAGAACATCCTGGTGTCAGACATGGAAATGAATGAGCAACAAGAATAGAG
CCAGGCGCGTGGGCAGGGCCAGGGCGGGAGGAGCTGGGGAGCTGGGGCCCTGGGTCAGTCTGGCCCCCCACCAGCTGCCTGTCCAG
TTGGCCTATGGAAGGGTGCCCTTGGGAGTCGCTGTTGGGAGCCGGAGCTGGGCAGAGCCTGGGCTGGTGGGGTGCCACCCTCCCAC
AAGGGCTGGGCTGAGACCCAGCTGAGTGCAGCGTGGCGTTTCCCTTTCTGGGGGGGCCTGAGGTCTTGTCACCTGGTCCTGTGCCC
CCACAGGAACCAGAGGTAGGATGGGAGGGGGAACGAGAGCCTCTTTCTCCCCAGAGCCCCCGGCCCAGGCCTGTTGATCCGCGCCC
CAGGACCCCCTTCTTTGCAGAGCCCGAGGAGCCTCCCCTGTCCCCTCGGGCAGATCTGTTGTGTCTCTCTTCCCACCTGGCAGCCT
CAGCTCTGTGCCCCTCACCCTGCTCCCTCTCGCCCCTTCTCTCCCACCCCTTCCTTCTGAGCCGGGCCCTGGGGATTGGGGAGCCC
TCTTGTTCCTGATGAGGGTCAGCTGAGGGGGCTGAGCATCCATCACTCCTGTGCCTGCTGGGGTGGCTGTGGGGCGTGGCAGGAGG
GGCCTAGGTGGGTTGGGCCTGAGAACCAGGGCACGGGTGTGGTGTCTGCTGGGCTGGAGATAAGACTGGGGAGAGACACCCCAACC
TCCCAGGGTGGGAGCTGGGCCGGGCTGGGATGTCATCTCCTGCCGGGCGGGGGAGGGCTCTGCCCCTGGAAGAGTCCCCTGTGGGG
ACCAAAATAAGTTCCCTAACATCTCCAGCTCCTGGCTCTGGTTTGGAGCAAGGGGAAGGGTTGCCAGAGTCCTGGGGGCCCCAGAG
GAGCAGGAGTCTGGGAGGGCCCAGAGTTCACCCTCTAGTGGATCCAGGAGGAGCAGCACCCGAGCCCTGGAGTGGCCCAGTACCCT
TCCAAGAGGCCACAGTCCCAGCCAGGACAAAGTATGCGGCCCATCCTGGTGCGACAGCGTGGGACAATGTGAACATGGACTCGAAG
ACATGGCCCTTTCTCTGTAGTTGATTTTTTAAATGTGCCATTATTGTTTTT

Following is a first CDK10 cDNA sequence (SEQ ID NO: 11).

>gi|32528262|ref|NM_003674.2| Homo sapiens cyclin-dependent kinase (CDC2-like) 10
(CDK10), transcript variant 1, mRNA
GTCTGCGCCTGCGCGCAAGAGAGGCGGGGCCAGCGCTCGGCATGGCGGAGCCAGATCTGGAGTGCGAGCAGATCCGTCTGAAGTGT
ATTCGTAAGGAGGGCTTCTTCACGGTGCCTCCGGAACACAGGGTGACCCACCGCCCCCAGCCTGGCCTGGCATTTCTTTGAGTTCA
GGAAGTGTGACAAGGATTTGGACACCCAGAAATAAGCGTGTCGAGAAGAGCACAAGCAGAGGATCTGGGACCATGCCGGAGTGTGA
AGGAGTTTGAGAAGCTGAACCGCATTGGAGAGGGTACCTACGGCATTGTGTATCGCGCCCGGGACACCCAGACAGATGAGATTGTC
GCACTGAAGAAGGTGCGGATGGACAAGGAGAAGGATGGCATCCCCATCAGCAGCTTGCGGGAGATCACGCTGCTGCTCCGCCTGCG
TCATCCGAACATCGTGGAGCTGAAGGAGGTGGTTGTGGGGAACCACCTGGAGAGCATCTTCCTGGTGATGGGTTACTGTGAGCAGG
ACCTGGCCAGCCTCCTCGAGAATATGCCAACACCCTTCTCGGAGGCTCAGGTCAAGTGCATCGTGCTGCAGGTGCTCCGGGGCCTC
CAGTATCTGCACAGGAACTTCATTATCCACAGGGACCTGAAGGTTTCCAACTTGCTCATGACCGACAAGGGTTGTGTGAAGACAGC
GGATTTCGGCCTGGCCCCGGCCTATGGTGTCCCAGTAAAGCCAATGACCCCCAAGGTGGTCACTCTCTCGTACCGAGCCCCTGAAC
TGCTGTTGGGAACCACCACGCAGACCACCAGCATCGACATGTGGGCTGTGGGCTGCATACTCGCCGAGCTGCTGGCGCACAGGCCT
CTTCTCCCCGGCACTTCCGAGATCCACCAGATCGACTTGATCGTGCAGCTGCTGGGCACGCCCAGTGAGAACATCTGGCCGGGCTT
TTCCAAGCTGCCACTGGTCGGCCAGTACAGCCTCCGGAAGCAGCCCTACAACAACCTGAAGCACAAGTTCCCATGGCTGTCGGAGG
CCGGGCTGCGCCTGCTGCACTTCCTGTTCATGTACGACCCTAAGAAAAGGGCGACGGCCGGGGACTGCCTGCAGAGCTCCTATTTC
AAGGAGAAGCCCCTACCCTGTGAGCCGGAGCTCATGCCGACCTTTCCCCACCACCGCAACAAGCGGGCCGCCCCAGCCACCTCCGA
GGGCCAGAGCAAGCGCTGTAAACCCTGACGGTGGGCCTGGCACACGCCTGTATTCCCACACCAGGTCTTCCGATCAGTGGTGTCTG
TGAAGGGTGCCGCGAGCCAGGCTGACCAGGCGCCCGGGATCCAGCTCATCCCCTTGGCTGGGAACATCCTCCACTGACTTCCTCCC
ACTGTCTGCCCTGAACCCACTGCTGCCCCCAGAAAAAGGCCGGGTGACACCGGGGGGCTCCCAGCCCCTGCACCCTGGAAGGGCAG
GTCTGGCGGCTCCATCCGTGGCTGCAGGGGTCTCATGTGGTCCTCCTCGCTATGTTGGAAATGTGCAACCACTGCTTCTTGGGAGG
AGTGGTGGGTGCAGTCCCCCCGCTGTCTTTGAGTTGTGGTGGACGCTGGCCTGGGATGAGAGGGCCCAGAAGACCTTCGTATCCCC
TCTCAGTCGCCCGGGGCTGTCCCGTGCATGGGTTGGCTGTGGGGACCCCAGGTGGGCCTGGCAGGACTCCAGATGAGGACAAGAGG
GACAAGGTATGGGGTGGGAGCCACAATTGAGGATACCCCGAGACTACCAGGAGAGCCCTGGGCTGGAGGCTGAGCTGCATCCCTGC
TCCCCACATGGAGGACCCAACAGGAGGCCGTGGCTCTGATGCTGAGCGAAGCTATAGGCTCTTGTTGGATAAAAGCTTTTTTAACA

Following is a seco-nd CDK10 cDNA sequence (SEQ ID NO: 12).

>gi|32528264|ref|NM_052987.2| Homo sapiens cyclin-dependent kinase (CDC2-like) 10
(CDK10), transcript variant 2, mRNA
GTCTGCGCCTGCGCGCAAGAGAGGCGGGGCCAGCGCTCGGCATGGCGGAGCCAGATCTGGAGTGCGAGCAGATCCGTCTGAAGTGT
ATTCCTGGGACGATGCCGGAGTGTGAAGGAGTTTGAGAAGCTGAACCGCATTGGAGAGGGTACCTACGGCATTGTGTATCGGGCCC
GGGACACCCAGACAGATGAGATTGTCGCACTGAAGAAGGTGCGGATGGACAAGGAGAAGGATGGCATCCCCATCAGCAGCTTGCGG
GAGATCACGCTGCTGCTCCGCCTGCGTCATCCGAACATCGTGGAGCTGAAGGAGGTGGTTGTGGGGAACCACCTGGAGAGCATCTT
CCTGGTGATGGGTTACTGTGAGCAGGACCTGGCCAGCCTCCTGGAGAATATGCCAACACCCTTCTCGGAGGCTCAGGTCAAGTGCA
TCGTGCTGCAGGTGCTCCGGGGCCTCCAGTATCTGCACAGGAACTTCATTATCCACAGGGACCTGAAGGTTTCCAACTTGCTCATG
ACCGACAAGGGTTGTGTGAAGACAGCGGATTTCGGCCTGGCCCGGGCCTATGGTGTCCCAGTAAAGCCAATGACCCCCAAGGTGGT
CACTCTCTGGTACCGAGCCCCTGAACTGCTGTTGGGAACCACCACGCAGACCACCAGCATCGACATGTGGGCTGTGGGCTGCATAC
TGGCCGAGCTGCTGGCGCACAGGCCTCTTCTCCCCGGCACTTCCGAGATCCACCAGATCGACTTGATCGTGCAGCTGCTGGGCACG
CCCAGTGAGAACATCTGGCCGGGCTTTTCCAAGCTGCCACTGGTCGGCCAGTACAGCCTCCGGAAGCAGCCCTACAACAACCTGAA
GCACAAGTTCCCATGGCTGTCGGAGGCCGGGCTGCGCCTGCTGCACTTCCTGTTCATGTACGACCCTAAGAAAAGGGCGACGGCCG
GGGACTGCCTGGAGAGCTCCTATTTCAAGGAGAAGCCCCTACGTCTTCCGATCAGTGGTGTCTGTGAAGGGTGCCGCGAGCCAGGC
TGACCAGGCGCCCGGGATCCAGCTCATCCCCTTGGCTGGGAACATCCTCCACTGACTTCCTCCCACTGTCTGCCCTGAACCCACTG
CTGCCCCCAGAAAAAGGCCGGGTGACACCGGGGGGCTCCCAGCCCGTGCACCCTGGAAGGGCAGGTCTGGCGGCTCCATCCGTGGC
TGCAGGGGTCTCATGTGGTCCTCCTCGCTATGTTGGAAATGTGCAACCACTGCTTCTTGGGAGGAGTGGTGGGTGCAGTCCCCCCG
CTGTCTTTGAGTTGTGGTGGACGCTGGCCTGGGATGAGAGGGCCCAGAAGACCTTCGTATCCCCTCTCAGTCGCCCGGGGCTGTCC
CGTGCATGGGTTGGCTGTGGGGACCCCAGGTGGGCCTGGCAGGACTCCAGATGAGGACAAGAGGGACAAGGTATGGGGTGGGAGCC
ACAATTGAGGATACCCCGAGACTACCAGGAGAGCCCTGGGCTGGAGGCTGAGCTGCATCCCTGCTCCCCACATGGAGGACCCAACA
GGAGGCCGTGGCTCTGATGCTGAGCGAAGCTATAGGCTCTTGTTGGATAAAAGCTTTTTTAACAGAAAAAAAAAAAAAAAAAAAA

Following is a third CDK10 cDNA sequence (SEQ ID NO: 13).

>gi|32528265|ref|NM_052988.2| Homo sapiens cyclin-dependent kinase (CDC2-like) 10
(CDK10), transcript variant 3, mRNA
GTCTGCGCCTGCGCGCAAGAGAGGCGGGGCCAGCGCTCGGCATGGCGGAGCCAGATCTGGAGTGCGAGCAGATCCGTCTGAAGTGT
ATTCGTAAGGAGGGCTTCTTCACGGTGCCTCCGGAACACAGGATCGGGCCCGGGACACCCAGACAGATGAGATTGTCGCACTGAAG
AAGGTGCGGATGGACAAGGAGAAGGATGGCATCCCCATCAGCAGCTTGCGGGAGATCACGCTGCTGCTCCGCCTGCGTCATCCGAA
CATCGTGGAGCTGAAGGAGGTGGTTGTGGGGAACCACCTGGAGAGCATCTTCCTGGTGATGGGTTACTGTGAGCAGGACCTGGCCA
GCCTCCTGGAGAATATGCCAACACCCTTCTCGGAGGCTCAGGTCAAGTGCATCGTGCTGCAGGTGCTCCGGGGCCTCCACTATCTG
CACAGGAACTTCATTATCCACAGGGACCTGAAGGTTTCCAACTTGCTCATGACCGACAAGGGTTGTGTGAAGACAGGTGGGTGCAA
CTTGGGCCAGGCCCTGTCCCTAGATGGCACTTGGTGACACACACTCCCCTCTCTGCTGCAGCGGATTTCGGCCTGGCCCGGGCCTA
TGGTGTCCCAGTAAAGCCAATGACCCCCAAGGTGGTCACTCTCTGGTACCGAGCCCCTGAACTGCTGTTGGGAACCACCACGCAGA
CCACCAGCATCGACATGTGGGCTGTGGGCTGCATACTGGCCGAGCTGCTGGCGCACAGGCCTCTTCTCCCCGGCACTTCCGAGATC
CACCAGATCGACTTGATCGTGCAGCTGCTGGGCACGCCCAGTGAGAACATCTGGCCGGCCTTTTCCAAGCTGCCACTGGTCGGCCA
GTACAGCCTCCGGAAGCAGCCCTACAACAACCTGAAGCACAAGTTCCCATGGCTGTCGGAGGCCGGGCTGCGCCTGCTGCACTTCC
TGTTCATGTACGACCCTAAGAAAAGGGCGACGGCCGGGGACTGCCTGGAGAGCTCCTATTTCAAGGAGAAGCCCCTACCCTGTGAG
CCGGAGCTCATGCCGACCTTTCCCCACCACCGCAACAAGCGGGCCGCCCCAGCCACCTCCGAGGGCCAGAGCAAGCGCTGTAAACC
CTGACGGTGGGCCTGGCACACGCCTGTATTCCCACACCAGGTCTTCCGATCAGTGGTGTCTGTGAAGGGTGCCGCGAGCCAGGCTG
ACCAGGCGCCCGGGATCCAGCTCATCCCCTTCGCTGGGAACATCCTCCACTGACTTCCTCCCACTGTCTGCCCTGAACCCACTGCT
GCCCCCAGAAAAAGGCCGGGTGACACCGGGGGGCTCCCAGCCCGTGCACCCTGGAAGGGCAGGTCTGGCGGCTCCATCCGTGGCTG
CAGGGGTCTCATGTGGTCCTCCTCGCTATGTTGGAAATGTGCAACCACTGCTTCTTGGGAGGAGTGGTGGGTGCAGTCCCCCCGCT
GTCTTTGAGTTGTGGTGGACGCTGGCCTGGGATCAGAGGGCCCAGAAGACCTTCGTATCCCCTCTCAGTCGCCCGGGGCTGTCCCG
TGCATGGGTTGGCTGTGGGGACCCCAGGTGGGCCTGGCAGGACTCCAGATGAGGACAACAGGGACAAGGTATGGGGTGGGAGCCAC
AATTGAGGATACCCCGAGACTACCAGGAGAGCCCTGGGCTGGAGGCTGAGCTGCATCCCTGCTCCCCACATGGAGGACCCAACAGG
AGCCCGTGGCTCTGATGCTGAGCGAAGCTATAGGCTCTTGTTGGATAAAAGCTTTTTTAACAGAAAAAAAAAAAAAAAAAAAA

Following is a FPGT cDNA sequence (SEQ ID NO: 14).

>gi|4503776|ref|NM_003838.1| Homo sapiens fucose-1-phosphate guanylyltransferase
(FPGT), mRNA
GCGTGCTGTGCGGCGCGGTCTCAGGGAAGGTGGGGCTATGGCAGCTGCTAGGGACCCTCCGGAAGTATCGCTGCGAGAAGCCACCC
AGCGAAAATTGCGGAGGTTTTCCGAGCTAAGAGGCAAACTTGTAGCACGTGGAGAATTCTGGGACATAGTTGCAATAACAGCGGCT
GATGAAAAACAGGAACTTGCTTACAACCAACAGCTGTCAGAAAAGCTGAAAAGAAAGGAGTTACCCCTTGGAGTTCAATATCACGT
TTTTGTGGATCCTGCTGGACCCAAAATTGGAAATGGAGGATCAACACTTTGTGCCCTTCAATGTTTGGAAAAGCTATATGGAGATA
AATGGAATTCTTTTACCATCTTATTAATTCACTCTGGTGGCTACAGTCAACGACTTCCAAATGCAAGTGCTCTGGGAAAAATTTTC
ACTGCTTTACCTCTTGGTAACCCCATTTATCAGATGCTAGAATTAAAGCTAGCCATGTACATTGATTTCCCCTTAAATATGAATCC
TGGAATTCTGGTTACCTGTGCAGATGATATTGAACTTTATAGTATTGGAGAATTTGAGTTTATTAGGTTTGACAAACCTGGCTTTA
CTGCTTTAGCTCATCCTTCTAGTTTGACGATAGGTACCACACATGGAGTATTTGTCTTAGATCCTTTTGATGATTTAAAACATAGA
GACCTTGAATACAGGTCTTGCCATCGTTTCCTTCATAAGCCCAGCATAGAAAAGATGTATCAGTTTAATGCTGTGTGTAGACCTGG
AAATTTTTGTCAACAGGACTTTGCTGGGGGTGACATTGCCGATCTTAAATTAGACTCTGACTATGTCTACACAGATAGCCTATTTT
ATATGGATCATAAATCAGCAAAAATGTTACTTGCTTTTTATGAAAAAATAGGCACACTGAGCTGTGAAATAGATGCCTATGGTGAC
TTTCTGCAGGCTTTGGGACCTGGAGCAACTGTGGAGTACACCAGAAACACATCACATGTCATTAAAGAAGAGTCAGAGTTGGTAGA
AATGAGGCAGAGAATATTTCATCTTCTTAAAGGAACATCACTAAATGTTGTTGTTCTTAATAACTCCAAATTTTATCACATTGGAA
CAACCGAAGAATATTTGTTTTACTTTACCTCAGATAACAGTTTAAAGTCAGAGCTCGGCTTACAGTCCATAACTTTTAGTATCTTT
CCAGATATACCAGAATGCTCTGGCAAAACATCCTGTATCATTCAAAGCATACTGGATTCAAGATGTTCTGTGGCACCTGGCTCAGT
TGTGGAGTATTCCAGATTGGGGCCTGATGTTTCAGTTGGGGAAAACTGCATTATTAGTGGTTCTTACATCCTAACAAAAGCTGCCC
TCCCCGCACATTCTTTTGTATGTTCCTTAAGCTTAAAGATGAATAGATGCTTAAAGTATGCAACTATGGCATTTGGAGTGCAAGAC
AACTTGAAAAAGAGTGTGAAAACATTGTCAGATATAAAGTTACTTCAATTCTTTGGAGTCTGTTTCCTGTCATGCTTAGATGTTTG
GAATCTTAAAGTTACAGAGGAACTGTTCTCTGGTAACAAGACATGTCTGAGTTTGTGGACTGCACGCATTTTCCCAGTTTGTTCTT
CTTTGAGTGACTCAGTTATAACATCCCTAAAGATGTTAAATGCTGTTAAGAACAAGTCAGCATTCAGCCTGAATAGCTATAAGTTG
CTGTCCATTGAAGAAATGCTTATCTACAAAGATGTAGAAGATATGATAACTTACAGGGAACAAATTTTTCTAGAAATCAGTTTAAA
AAGCAGTTTGATGTAGAGATATTTTAAATATTGTACACTTTGCCTTTTTGAGTAACATTCCAGAGATAGGTATTTTTGGTAGGCTG
TTTCACTGAACTCAGTTAATGAAAACTGTATTAACATAATTGTTGTAGCATAATATTAATAGTGCAAAAGTACATATAAGTCATTT
TGATGAAAAATATTCCAAGACTAAGTTGAGAAAAGAGATACTATTTTGGATGTGTATCAGTATTTTTGTTTTTAATAATGATTGAT
TTGTGGAGCATTGTTTTTTCACATAATTAGTTTTAAAGGTAATTTTCTAAGCATACCTTTGGAATTTTTCCATCTTTTTTGAGGCT
TTTGGTCCAGTGAAGTTCTAAGTATTCACTGGCACTTCTCTCCTCAACTGTAATTCTATTTTTAATAATAAAAATGGCATACTGTA
GGGTCTTCAGAGTAGTGTAGGAATACTGTAGAAATACTTTTTCAGAAACGAATCCATAGCTGACAAATTCACTCAGTGCCCAATAT
ATTGTGATTATTTTCGTTGATAAAGAACTAGATACAAAGACCTCTGAAATTGATGATAAAATTTGTATCTCATTAATTTTATCAAA
ATGAAACTAAAAGTACATATGTATTATACACTTGAACATGTGTTTGTATATCTTTAAAATTTTCCTTTATGTCCATTCCATAGGAA
GACACACATATGCACACAAAACTCAGTTATCTGTGGGGGAAGTGGTAGTAATAATTGAGATTCATCAATAATCATATAATTTCACT
ATAGACATTACTTGCATTATCTCCTATCAGTCCTTCTCACAAAACTTCAATTACCAGATGAACTGACTTTAGTTTTCATCTTATTT
TTTGTCCACATGCTGGTGATGCTGAGAAACAATAATTGGCCCAAATGCAGACATCAAGAAGCAGGCAGGAAIATGGAAAAACTGAG
ATAATACACAGGTGATAAACAGTTTCGAGAAAATAAAAATTGGCTAGAATCTCAAGTTATGTGTTTCTATATTGGTATAAGCATAT
AAGTGAAAAGTCTTATAAGTGTAATAGAGAAAAGATTTGTCAGTGTGTTTTTTTAAATGAAATAACTAGTCTGTGCTACTTTATGT
CAATATAAAAATTGGTAAACTAGAAGTAACTTGTCCACAACCCTCAGTTATGATACTTATGTGCGTGTTTTTTTTTCCAAAGTTTT
TCCCAAGGAGAAGATACAAATATATGGTAGCTATTGTTTAAAAATGATTGATTTACTTGCAGATTTTTCAGAGGATGTTATGTCTT
TGTCATTCATTAAATGTTCAAAATTGTAGTTTTAAAAAAAAAAAAAAA

Following is a CARK cDNA sequence (SEQ ID NO: 15).

>gi|7705747|ref|NM_015978.1| Homo sapiens cardiac ankyrin repeat kinase (CARK), mRNA
AATCTTATAACTTGAAGAACTGCCCTGGAGAAAGGAAGAAACTTATAATAAATGGGAAATTATAAATCTAGACCAACCCAAACTTG
TACTGATGAATGGAACAAAAAAGTCAGTGAATCATATGTTATCACAATAGAAAGATTAGAAGATGACCTGCAGATCAAGGAAAAAG
AACTGACAGAACTAAGGAATATATTTGGCTCTCATCAAGCCTTCAGTAAAGTCAATTTAAATTACCGCACTCAAAATGGGCTGTCT
CTACTTCATTTATGTTGCATTTCTGCAGCCAAGAAATCACATATTCGAACTCTTATGTTGAAAGGGCTCCGCCCATCTCGACTGAC
AAGAAATGGATTTACAGCCTTGCATTTAGCAGTTTACAAGGATAATGCAGAATTGATCACTTCTCTGCTTCACAGTGGAGCTGATA
TACAGCAGGTTGGATACGGTGGCCTCACTCCCCTCCATATTGCTACAATAGCTGGCCACCTAGAGGCTGCTGATGTGCTGTTGCAA
CATGGAGCTAATGTCAATATTCAAGATGCAGTTTTTTTCACTCCATTGCATATTGCAGCGTACTATGGACATGAACAGGTAACTCG
CCTTCTTTTGAAATTTGGTGCTGATGTAAATGTAAGTGGTGAAGTTGGAGATAGACCCCTCCACCTAGCATCTGCAAAAGGATTCT
TGAATATTGCAAAACTCTTGATGGAAGAAGGCAGCAAAGCACATGTGAATGCTCAAGATAATGAAGACCATGTCCCACTCCATTTC
TGTTCTCGATTTGGACACCATGATATAGTTAAGTATCTGCTGCAAAGTGATTTGGAAGTTCAACCTCATGTTGTTAATATCTATGG
AGATACCCCCTTACACCTGGCATGCTACAATGGCAAATTTGAAGTTGCCAACGAAATCATCCAAATATCAGGAACAGAAAGTCTGA
CTAAGGAAAACATCTTCAGTGAAACAGCTTTTCATAGTGCTTGTACCTATGGCAAGAGCATTGACCTAGTCAAATTTCTTCTTGAT
CAGAATGTCATAAACATCAACCACCAAGGAAGGGATGGGCACACTGGATTACACTCTGCTTGCTACCACGGTCACATTCGCCTGGT
TCAGTTCTTACTGGATAATGGAGCTGATATGAATCTAGTGGCTTGTGATCCCAGCAGGTCTAGTGGTGAAAAAGATGAGCAGACAT
GTTTGATGTGGGCTTATGAAAAAGGGCATGATGCCATTGTCACACTCCTGAAGCATTATAAGAGACCACAAGATGAATTGCCCTGT
AATGAATATTCTCAGCCTGGAGGAGATGGCTCCTATGTCTCTGTTCCATCACCCTTGGGGAAGATTAAAAGCATGACAAAAGAGAA
GGCAGATATTCTCCTCCTAAGAGCTGGATTGCCTTCACATTTCCATCTTCAGCTCTCAGAAATTGAGTTCCATGAGATTATTGGCT
CAGGTTCTTTTGGGAAAGTATATAAAGGACGATGCAGAAATAAAATAGTGGCTATAAAACGTTATCGAGCCAATACCTACTGCTCC
AAGTCAGATGTGGATATGTTTTGCCGAGAGGTGTCCATTCTCTGCCAGCTCAATCATCCCTGCGTAATTCAGTTTGTGGGTGCTTG
CTTGAATGATCCCAGCCAGTTTGCCATTGTCACTCAATACATATCAGGGGGTTCTCTGTTCTCCCTCCTTCATGAGCAGAAGAGGA
TTCTTGATTTGCAGTCTAAATTAATTATTGCAGTAGATGTTGCCAAAGGCATGGAGTACCTTCACAACCTGACACAGCCARTTATA
CATCGTGACTTGAACAGTCACAATATTCTTCTCTATGAGGATGGGCATGCTGTGGTGGCAGATTTTGGAGAATCAAGATTTCTACA
GTCTCTGGATGAAGACAACATGACAAAACAACCTGGGAACCTCCGTTGGATGGCTCCTGAGGTGTTCACGCAGTGCACTCGGTACA
CCATCAAAGCAGATGTCTTCAGCTATGCTCTGTGTCTGTGGGAAATTCTCACTGGCGAAATTCCATTCGCTCATCTCAAGCCAGCG
GCTGCGGCAGCAGACATGGCTTACCACCACATCAGACCTCCCATTGGCTATTCCATTCCCAAGCCCATATCATCTCTGCTGATACG
AGGGTGGAACGCATGTCCTGAAGGAAGACCCGAATTTTCTGAAGTTGTCATGAAGTTAGAAGAGTGTCTCTGCAACATTGAGCTGA
TGTCTCCTGCATCAAGTAACAGCAGTGGGTCTCTCTCACCTTCTTCTTCTTCTGATTGCCTGGTGAACCGGGGAGGACCTGGCCGG
AGTCATGTGGCAGCATTAAGAAGTCGTTTCGAATTGGAATATGCTCTAAATGCAAGGTCCTATGCTGCTTTGTCCCAAAGTGCTGG
ACAATATTCCTCTCAAGGTCTGTCTTTGGAGGAGATGAAAAGAAGTCTTCAATACACACCCATTGACAAATATGGCTATGTATCCG
ATCCCATGAGCTCAATGCATTTTCATTCTTGCCGAAATAGTAGCAGCTTTGAGGACAGCAGCTGACAGCATTCGGCGTATACCTAR
GGAGAGTTTTTTCCCCGAACTGACAGCAACGATTCCAACCACGGCAAGCTGGCTTCCAACTATAACATTTTACTCTCAAAGGTCTC
CTTAAATTGGGCTTGTTTTTACTTGTCCTATTTAATTCCCCACTATTAGCAGGCTTTGGATTTGTGCCTAAGGAATAATATGCAAA
AGAACCAAGACACAATGTATATGAAGAATTGTTTTTAATTTTGTAAATTAAAAAAAAATTTAGATCGTTACTTGGAAATGGAGCCT
AAGTCTGTGGTGGACAGATAATAATTATGTTTTCCTGGGCTGAATTATGTAGACTTGTGTTTGACAGCTATGGGTTTATTTCTTAG
AACATTGTTCATTTTCTTTTCTCATTATGTTACTTCTAGTGTTCACCTCTGTGATTAAAGATTCTTTGGTGAAATAG

Following is a PCLO cDNA sequence (SEQ ID NO: 16).

>gi|37538611|ref|XM_168530.2| Homo sapiens piccolo (presynaptic cytomatrix protein)
(PCLO), mRNA
ATGACATCCTCTGGAGCCCAGAAAAAAGTTAAAAGAACTCTGCCAAATCCACCTCCTGAGGAGATTTCCACAGGAACTCAATCCAC
ATTCAGCACAATGGGCACAGTTTCCAGGAGAAGGATCTGCAGAACCAACACAATGGCACGAGCCAAGATTCTCCAGGACATAGACA
GAGAGCTTGATCTTGTGGAAAGGGAGTCTGCAAAACTTCGAAAGAAACAAGCAGAGCTTGATGAAGAAGAAAAGGAGATTGATGCT
AAGCTACGATACCTGGAAATGGGAATTAACAGGAGGAAAGAGGCCCTATTAAAGGAGAGAGAAAAGAGAGAACGAGCCTACCTCCA
GGGAGTAGCTGAGGATCGTGATTACATGTCTGACAGTGAAGTGAGTAGCACAAGACCAACCCGAATAGAAAGTCAGCATGGCATTG
AGCGACCAAGAACTGCTCCCCAAACTGAATTCAGCCAGTTTATACCACCACAAACCCAAACAGAATCTCAACTAGTTCCTCCGACA
AGTCCTTACACACAATACCAGTACTCTTCCCCTGCTCTTCCTACCCAAGCACCCACCTCATACACTCAACAGTCTCATTTTGAGCA
ACAAACTTTGTACCATCAGCAAGTTTCACCTTATCAGACTCAGCCAACATTCCAAGCTGTGGCAACAATGTCCTTCACACCTCAAG
TTCAACCTACACCAACCCCACAGCCTTCTTATCAGTTACCTTCACAGATGATGGTGATACAACAGAAGCCACGGCAAACTACATTA
TATTTGGAGCCCAAGATAACCTCAAACTATGAAGTGATTCGCAACCAACCCCTTATGATAGCACCTGTTTCTACGGATAACACATT
TGCTGTTTCCCATCTTGGTAGTAAGTACAATAGTTTAGACTTGAGAATAGGTTTGGAGGAAAGAAGTAGCATGGCAAGCAGTCCAA
TATCAAGCATATCTGCAGATTCTTTCTATGCAGATATTGATCACCATACTCCACGAAATTATGTCCTAATTGACGACATTGGAGAG
ATCACCAAAGGAACAGCGGCATTAAGCACCGCATTTAGCCTTCATGAAAAGGATCTGTCAAAAACAGACCGTCTCCTTCGAACCAC
TGAGACACGCCGGTCTCAAGAAGTGACAGATTTCCTAGCACCTTTACAGTCTTCCTCTAGATTGCATAGTTATGTGAAGGCGGAGG
AAGACCCAATGGAGGATCCTTACGAGTTAAAGCTTCTGAAACATCAGATTAAACAGGAATTTCGTAGAGGGACAGAGAGCTTAGAT
CACCTTGCTGGTCTTTCTCATTATTACCATGCTGATACTAGCTACAGACATTTTCCAAAATCTGAGAAGTATAGCATCAGTAGACT
CACACTTGAAAAACAAGCAGCAAAACAACTGCCAGCAGCCATACTTTATCAAAAGCAGTCAAAGCATAAGAAATCACTAATTGACC
CTAAAATGTCAAAATTTTCACCTATTCAAGAAACTAGAGACCTTGAACCTGATTATTCAAGCTATATGACTTCTAGCACTTCATCT
ATTGGTGGCATTTCCTCCAGGGCAAGGCTCCTTCAAGATGACATCACTTTTGGCCTCAGAAAAAATATTACAGACCAACAAAAATT
TATGGGATCTTCTCTTGGCACAGGACTGGGCACATTAGGAAATACCATACGCTCAGCTCTGCAGGATGAAGCGGATAAGCCATACA
GTAGTGGCAGCAGGTCCAGACCTTCCTCCAGACCTTCCTCTGTCTATGGGCTTGATTTATCAATTAAAAGGGATTCTTCTAGCTCT
TCCCTAAGACTGAAAGCTCAAGAGGCTGAAGCTCTAGATGTTTCCTTTAGTCATGCATCATCCTCTGCCAGAACTAAGCCGACCAG
TTTGCCAATTAGTCAAAGTAGAGGAAGAATACCAATTGTGGCCCAGAATTCTGAAGAAGAAAGCCCACTCAGTCCTGTTGGCCAGC
CAATGGGAATGGCCAGGGCTGCAGCTGGACCCCTGCCACCAATATCTGCAGACACCAGGGATCAGTTTGGATCAAGCCACTCATTG
CCTGAAGTTCAGCAACACATGAGGGAAGAATCACGGACTCGAGGCTATGACCGTGACATAGCATTCATCATGGATGACTTCCAACA
TGCCATGTCAGACAGTGAAGCCTATCATCTGCGTCGTGAGGAAACAGATTGGTTTGATAAACCCAGGGAGTCTCGTTTGGAAAATG
GACATGGTCTGGACCGAAAACTGCCGGAAAGATTGGTCCACTCTAGACCACTCAGTCAACATCAAGAGCAAATTATACAGATGAAC
GGGAAAACTATGCACTACATCTTTCCTCACGCAACGATAAAAATAACAAGAGACTCAAAGGATCACACAGTTTCAGGTAATGGATT
AGGAATTAGAATTGTGGGTGGTAAAGAAATCCCGGGACATAGTGGAGAAATTCGAGCCTATATTGCCAAGATTCTTCCTGGGGGAA
GTGCGGAACAGACCGGGAAGCTTATGGAAGGGATGCAAGTATTGGAATGGAATGGAATTCCCTTGACTTCTAAAACATATGAAGAA
GTTCAGAGTATCATTAGTCAGCAAAGTGGGGAACCAGAAATATGTGTAAGACTGGACCTCAATATGCTATCAGATTCTGAAAATTC
CCAGCATCTGGAACTTCATGAGCCACCAAAAGCTGTGGATAAGGCGAAATCCCCAGGGGTTGATCCTAAGCAGTTGGCAGCAGAAC
TCCAGAAGGTTTCACTACAGCAGTCACCGCTGGTTCTGTCATCAGTTGTTGAAAAAGGATCTCATGTTCATTCAGGTCCTACATCA
GCAGGATCCAGTTCCGTTCCCAGCCCTGGGCAACCAGGGTCCCCCTCAGTGAGCAAAAAGAAGCACGGCAGCAGCAAGCCTACCGA
TGGAACAAAGGTTGTCTCTCATCCAATTACAGGAGAAATTCAGCTTCAAATTAACTATGATCTTGGAAATCTCATAATACATATTC
TCCAAGCAAGAAATCTTGTTCCTCGAGACAACAATGGTTATTCTGACCCTTTTGTGAAAGTGTACCTTCTTCCAGGGAGAGGTCAA
GTCATGGTTGTCCAGAATGCAAGTGCTGAGTACAAGAGAAGGACTAAACATGTCCAGAAA~GTCTTAATCCTGAGTGGAATCAAAC
AGTAATTTATAAAAGTATTTCCATGGAACAGCTCAAGAAGAAAACACTGGAGGTGACAGTTTGGGATTATGATAGATTTTCATCCA
ACGACTTCCTTGGGGAGGTATTGATTGATTTATCTAGCACATCTCACCTCGATAACACTCCAAGGTGGTATCCTCTCAAAGAACAG
ACTGAAAGCATTGATCATGGCAAGTCTCATTCCAGTCAGAGCAGCCAGCAGTCCCCAAAGCCATCTGTTATCAAAAGCAGAAGCCA
TGGTATCTTCCCTGACCCATCAAAGGACATGCAGGTTCCCACCATTGAGA~ATCCCATAGTAGTCCTGGTAGCTCAAAATCATCAT
CAGAAGCCCATCTCCGTTCTCATGGACCATCTCCCAGTCAAAGCAAAACCAGCGTCACTCAGACCCACCTGGAAGATGCAGGGGCT
GCCATAGCTGCTGCCGAAGCTCCCGTGCAACAACTCCGCATTCAACCAAGTAAAAGACGCAAATAAATTCCTCAGCATGGCAGCTT
AATGTTCATCTGTTGCCTTTCTTTCCTGCTGTCCTTTCCTGTTTGCTTTCAGTTTTCAACATCCTCTGCTCACCCTGTTCTCTGTC
CCTTTGTCTGTGTAAGAACGATATAAATACATTAATATGCTCTTTCTTATTTAGATTTTTTTTATTTACATAGACTGAAATAAAAC
TGGCTGTTTCTCCTTTGTTTCCACCATCCATCCAACCTGGCTCATAGCATTTGATACAGTGTCTGTGATGTTTGGAAGCAAAGCAA
TGTTGTGTGTCCTTTTTGTTTGCGCTTAAATATCTTTTAGAATACAGAAATTATAAGCACAAGTGGCTGCCAGTTTTCCAGGACTT
ATACCAAAAAAAAAAAAGTGCACACATATAAGTATTTAGAGCAGATTTGCAATTAGATTCTTGTATCTGAAAACACTCAAAAAGTC
TAGGCAGAAGATAATGCAACCAACAGTCAACTTCTAGAACCTTGGAGGAAAAGCCATTTCAAGACAGTGCTTTAAAACACTAACAA
ATTTTGCTTCTGAAATATGGTAGCTTTCCATGTGCAAAAAATTGTAGTCTTCTGAAAAATCTGTGAAGAAAATCTTATTTCTTACA
TTCTCAAAAAACTACATTAAATTAATATATGTATAAGCATATGTGATAAGGCAATGCAACCTCACTGAGGAGCACAATTGGGTCCA
TTCTGTTGGATCACACTTTGTATGTGATGACAGATTTTACAGATATTTTTTCTAACCATTGGAAATCAAGATATACTATGTCTGGT
AGCTATAAGTCAATCAAAGTATAACAATTGACCTATCTTAACTGCTGTATTTTCATTTCATTTGTGAAAATTTAGTCGATTGATAT
AATTATATAAAAACTTAATATGTGCAAATATTTCACAAATGTAGGTACCCTGTTACTTCTAAAAGGCATTAGCCAGGCAATGGAGA
TAGATCATTGTTCTCTCCATCTCCCTAGTCATCATTTTCAAAGAAACAGCCAGTTTTAAACCCCTTTAGTGCTGTGCATGTGGTGT
CATTTTGGCATTTGAAAACACAACCTATTTAAATAGAGCAAAAGAGCACTTCTGTTTGCATGAATAACATTTAAGCTGGTTCCATC
TGAGGATACATACTCTGGGTTTCCATTTCACCCACACTGCCACAGCAGCACATAAAAGCGGTCAGTCTAATCATGCCAGGAAGCAG
CACAGACACAGCATAGCAGGAGTCCTCCCCATTCAAAGAACTCAGTCTGATAATCTGCCTCCACCAGCCAATGGCAAC
CAAGACCAAAGCCAGCTAGCCCTCAGGAAGGTGATGTCTGATGGACCAGTCAAGCCAGAAGGAGGTGAGCAGAAAGGCCCTGTTCA
AAGCACACAGCAAGAACATCCCCAAATTTAAAGCTCTCATCTTTAATTTTTAGAGATTGTCTGATAAAATATGTTTATCCACTTTT
TAATGTGCTTTTGTGGGCATGGCATTTCAGGACTTCATCATCACCATGTAAGTCCCAAGATCAGTCCCGTAATATATTGACTACAG
TGATTTGTATTTTTCTATGCACTGGGCATCTTTTGACTTTTGTTGGGAAATAGATTAACAATATTCATATTGCAGAATGGAATGCA
TGCTACTAACCTAATCTGTTCAAGCATGACTATAAATATGTTTCATTGTGATAAAAGTTTAAATTGCTCACAGTTCAAGTTAGCCT
ACCAGTTGGAATGTGGATCAGTGTTTTTTGCCTATTATTTCAAGTGTAACTTTTAAAAATTTAGTGGATTTTGAAAATTCTTAGAG
GAAAGTAAAGGAAAAATTGTTAATGCACTCATTTACCTTTACATGGTGAAAGTTCTCTCTTGATCCTACAAACAGACATTTTCCAC
TCGTGTTTCCATAGTTGTTAAGTGTATCAGATGTGTTGGGCATGTGAATCTCCAAGTGCCTGTGTAATAAATAAAGTATCTTTATT
TCATTCAT2

Following is a REPS2 cDNA sequence (SEQ ID NO: 17).

>gi|4758943|ref|NM_004726.1| Homo sapiens RALBP1 associated Eps domain containing 2
(REPS2), Mrna
CCGGGAGGAAGCGGCCGCGCGGCAGCTGCGGGGCGTGGGGGTGGTGGTGGCGGCGGCGGTGGTGGCGGCGGCGGCGGCGGCGGCAG
CTGAGGCCGAGGAGGCGGTGGCTGTGGCGGACGCAGCAGCACCCCAGCTAGGGATAGGGCTCCGCCGCGCCCCCTTGCTGGCCCCA
TGGAGGCGGCAGCGGCGGCGGCGGCGGCGGCAGCGGCAGCGGCAGCGGCGGGCGGGGGCTGTGGCTCCGGGCCGCCGCCGCTGCTG
CTGAGCGAGGGCGAGCAGCAGTGCTACTCCGAGCTCTTCGCGCGCTGTGCCGGCGCCGCGGGCGGGGGCCCCGGGTCTGGGCCCCC
CGAGGCCGCCAGAGTCGCCCCCGGCACGGCCACTGCGGCCGCCGGCCCCGTGGCTGACCTGTTTCGGGCATCGCAGCTGCCCGCCG
AGACGCTGCACCAGATCACACAACTGTGTGGTGCAAAGCGGGTTGGTTATTTTGGTCCAACACAGTTTTACATTGCCCTGAAATTA
ATTGCTGCAGCACAATCTGGCCTCCCGGTACGGATAGAGAGTATTAAATGTGAATTGCCTCTGCCTCGCTTTATGATGTCAAAGAA
TGATGGTGAGATACGATTTGGGAACCCAGCTGAGCTGCATGGAACTAAGGTTCAGATTCCATATTTAACTACAGAAAAAAATTCCT
TCAAAAGAATGGACGATGAGGATAAACAGCAGGAAACACAGTCTCCCACGATGTCACCCCTCGCCTCCCCTCCTTCTTCCCCGCCT
CATTACCAGAGGGTGCCCTTGAGCCATGGCTACAGCAAACTGCGGAGCAGCGCAGAACAGATGCATCCAGCACCTTATGAAGCTAG
GCAGCCCCTTGTCCAGCCCGAGGGATCCTCATCAGGGGGCCCAGGAACCAAGCCCCTTCGGCATCAGGCTTCCCTTATCCGGTCCT
TTTCAGTGGAGAGGGAACTACAGGATAACAGCAGTTACCCCCACGAACCCTGGAGGATAACAGAAGAACAGCGCGAGTACTATGTC
AATCAGTTCCGATCCCTTCAGCCACACCCAAGCTCTTTCATTTCAGGTTCTGTGGCCAAGAACTTCTTCACCAAATCAAAGCTTTC
CATTCCAGAACTCTCCTATATATGGGAGCTTAGTGATGCTGACTGTGATGGAGCCCTGACCCTGCCTGAGTTCTGTGCTGCGTTTC
ATCTCATTGTGGCTCGGAAGAACGGCTACCCATTGCCTGAGGGCCTCCCTCCAACTCTGCAGCCAGAATACCTGCAGGCAGCTTTT
CCTAAGCCCAAATGGGACTGTCAATTATTTGATTCTTATTCTGAGTCACTGCCGGCAAATCAACAACCTCGTGACTTGAATCGGAT
GGAGAAGACATCTGTTAAAGACATGGCTGACCTTCCTGTCCCTAACCAGGATGTAACTAGTGATGACAAACAAGCTTTGAAAAGTA
CTATCAATGAAGCCTTACCAAAGGACGTGTCTGAGGATCCAGCAACTCCCAAGGATTCCAACAGTCTCAAAGCAAGACCAAGATCC
AGATCTTACTCTAGCACCTCCATAGAAGAGGCCATGAAAAGGGGCGAGGACCCTCCCACCCCGCCACCTCGGCCACAGAAAACCCA
TTCCAGAGCCTCCTCCTTGGATCTGAATAAAGTCTTCCAGCCCAGTGTGCCAGCTACCAAGTCAGGATTGTTACCCCCACCACCTG
CGCTCCCTCCAAGACCTTGTCCATCACAGTCTGAACAAGTGTCGGAGGCCGAGTTACTCCCACAGCTGACCAGAGCCCCATCCCAG
GCTGCAGAAAGTAGTCCAGCAAAGAAGGATGTACTGTATTCTCAGCCACCATCAAAGCCCATTCGTAGGAAATTCAGACCAGAAAA
CCAAGCTACAGAAAACCAAGAGCCTTCCACTGCTGCAAGTGGGCCAGCTTCTGCGGCAACCATGAAACCGCATCCAACAGTCCAAA
AGCAGTCTTCCAAACAGAAGAAGGCCATTCAAACTGCTATCCGCAAAAATAAAGAGGCAAACGCAGTGCTGGCTCGGCTGAACAGT
GAGCTCCAGCAGCAGCTCAAGGAGGTTCATCAAGAACGAATTGCATTGGAAAACCAATTGGAACAACTTCGTCCGGTCACTGTGTT
GTGACCCCCCCATGGTTCAAGTGACAGTGGGTGACCTTGTCTGCCAAGATCTTTCTTTTGAATGTTTTGAACCCAACTACTTGTCA
TAGATGTTTGACTGTGTCAAAAGCTGTGAGCAGCAAAATATAATCCATA

Following are NRP1, NID2, ENDO180, CDK10, FPGT, CARK, PCLO and REPS2 amino acid sequences (SEQ ID NO: 18, 19, 20, 21, 22, 23, 24, 25, 26 and 27, respectively). Following is a NRP1 amino acid sequence (SEQ ID NO: 18).

>gi|4505457|ref|NP_003864.1| neuropilin 1 [Homo sapiens]
MERGLPLLCAVLALVLAPAGAFRNDECGDTIKIESPGYLTSPGYPHSYHPSEKCEWLIQA
PDPYQRIMINFNPHFDLEDRDCKYDYVEVFDGENENGHFRGKFCGKLAPPPVVSSGPFLF
IKFVSDYETHGAGFSIRYEIFKRGPECSQNYTTPSGVIKSPGFPEKYPNSLECTYIVFAPK
MSEIILEFESFDLEPDSNPPGGMFCRYDRLEIWDGFPDVGPHIGRYCGQKTPGRIRSSSGI
LSMVFYTDSAIAKEGFSANYSVLQSSVSEDFKCMEALGMESGEIHSDQITASSQYSTNW
SAERSRLNYPENGWTPGEDSYREWIQVDLGLLRFVTAVGTQGAISKETKKKYYVKTYK
IDVSSNGEDWITIKEGNKPVLFQGNTNPTDVVVAVFPKYLITRFVRIKPATWETGISMRF
EVYGCKITDYPCSGMLGMVSGLISDSQITSSNQGDRNWMPENLRLVTSRSGWALPPAPH
SYINEWLQIDLGEEKIVRGIIIQGGKHRENKVFMRKFKIGYSNNGSDWKMIMDDSKRKA
KSFEGNNNYDTPELRTFPALSTRFIRIYPERATHGGLGLRMELLGCEVEAPTAGPTTPNG
NLVDECDDDQANCHSGTGDDFQLTGGTTVLATEKPTVIDSTIQSEFPTYGFNCEFGWGS
HKTFCHWEHDNHVQLKWSVLTSKTGPIQDHTGDGNFIYSQADENQKGKVARLVSPVV
YSQNSAHCMTFWYHMSGSHVGTLRVKLRYQKPEEYDQLVWMAIGHQGDHWKEGRV
LLHKSLKLYQVIFEGEIGKGNLGGIAVDDISINNHISQEDCAKPADLDKKNPEIKIDETGS
TPGYEGEGEGDKNISRKPGNVLKTLEPILITIIAMSALGVLLGAVCGVVLYCACWHNGM
SERNLSALENYNFELVDGVKLKKDKLNTQSTYSEA

The following Table shows polypeptide domain regions in a NRP1 amino acid sequence.

| | | AA | | |
|---|---|---|---|---|
| Prediction | Accession | start | AA | Domain |
| method | id | site | end | description |
| prints | PR00020 | 692 | 708 | MAM_domain |
| prints | PR00020 | 720 | 731 | MAM_domain |
| prints | PR00020 | 769 | 783 | MAM_domain |
| prints | PR00020 | 788 | 801 | MAM_domain |
| Pfam | PF00431 | 27 | 138 | CUB_domain |
| Pfam | PF00431 | 147 | 262 | CUB_domain |
| Pfam | PF00754 | 278 | 421 | FA58_C |
| Pfam | PF00754 | 434 | 580 | FA58_C |
| Pfam | PF00629 | 650 | 811 | MAM_domain |
| prosite | PS00740 | 690 | 730 | MAM_domain |
| prosite | PS01285 | 314 | 343 | FA58_C |
| prosite | PS01285 | 470 | 502 | FA58_C |
| prosite | PS01286 | 408 | 424 | FA58_C |
| prosite | PS01286 | 566 | 583 | FA58_C |
| pfscan | PS01180 | 27 | 141 | CUB_domain |
| pfscan | PS01180 | 147 | 256 | CUB_domain |

Following is a NID2 amino acid sequence (SEQ ID NO: 19).

>gi|33469989|ref|NP_031387.2| nidogen 2; osteonidogen [Homo sapiens]
MEGDRVAGRPVLSSLPVLLLLQLLMLRAAALHPDELFPHGESWGDQLLQEGDDESSAV
VKLANPLHFYEARFSNLYVGTNGIISTQDFPRETQYVDYDFPTDFPAIAPFLADIDTSHGR
GRVLYREDTSPAVLGLAARYVRAGFPRSARFTPTHAFLATWEQVGAYEEVKRGALPSG
ELNTFQAVLASDGSDSYALFLYPANGLQFLGTRPKESYNVQLQLPARVGFCRGEADDL
KSEGPYFSLTSTEQSVKNLYQLSNLGIPGVWAFHIGSTSPLDNVRPAAVGDLSAAHSSVP
LGRSFSHATALESDYNEDNLDYYDVNEEEAEYLPGEPEEALNGHSSIDVSFQSKVDTKP
LEESSTLDPHTKEGTSLGEVGGPDLKGQVEPWDERETRSPAPPEVDRDSLAPSWETPPP
YPENGSIQPYPDGGPVPSEMDVPPAHPEEEIVLRSYPASGHTTPLSRGTYEVGLEDNIGS
NTEVFTYNAANKETCEHNHRQCSRHAFCTDYATGFCCHCQSKFYGNGKHCLPEGAPH
RVNGKVSGHLHVGHTPVHFTDVDLHAYIVGNDGRAYTAISHIPQPAAQALLPLTPIGGL
FGWLFALEKPGSENGFSLAGAAFTHDMEVTFYPGEETVRITQTAEGLDPENYLSIKTNIQ
GQVPYVPANFTAHISPYKELYHYSDSTVTSTSSRDYSLTFGANQTWSYRIHQNITYQVC
RHAPRHPSFPTTQQLNVDRVFALYNDEERVLRFAVTNQIGPVKEDSDPTPGNPCYDGSH
MCDTTARCHPGTGVDYTCECASGYQGDGRNCVDENECATGFHRCGPNSVCINLPGSY
RCECRSGYEFADDRHTCILITPPANPCEDGSHTCAPAGQARCVHHGGSTFSCACLPGYA
GDGHQCTDVDECSENRCHPAATCYNTPGSFSCRCQPGYYGDGFQCIPDSTSSLTPCEQQ
QRHAQAQYAYPGARFHIPQCDEQGNFLPLQCHGSTGFCWCVDPDGHEVPGTQTPPGST
PPHCGPSPEPTQRPPTICERWRENLLEHYGGTPRDDQYVPQCDDLGHFLPLQCHGKSDFC
WCVDKDGREVQGTRSQPGTTPACIPTVAPPMVRPTPRPDVTPPSVGTFLLYTQGQQIGY
LPLNGTRLQKDAAKTLLSLHGSIIVGIDYDCRERMVYWTDVAGRTISRAGLELGAEPETI
VNSGLISPEGLAIDHIRRTMYWTDSVLDKIESALLDGSERKVLFYTDLVNPRAIAVDPIR
GNLYWTDWNREAPKIETSSLDGENRRILINTDIGLPNGLTFDPFSKLLCWADAGTKKLE
CTLPDGTGRRVIQNNLKYPFSIVSYADHFYHTDWRRDGVVSVNKHSGQFTDEYLPEQR
SHLYGITAVYPYCPTGRK

The following Table shows domain regions in a NID2 amino acid sequence.

| | | AA | | |
|---|---|---|---|---|
| Prediction | Accession | start | AA | DOMAIN |
| method | id | site | end | DESCRIPTION |
| Pfam | PF00008 | 763 | 799 | EGF_like |
| Pfam | PF00008 | 805 | 842 | EGF_like |
| Pfam | PF00008 | 852 | 890 | EGF_like |
| Pfam | PF00008 | 896 | 929 | EGF_like |
| Pfam | PF00086 | 940 | 1005 | Thyroglobulin_1 |
| Pfam | PF00086 | 1019 | 1084 | Thyroglobulin_1 |
| Pfam | PF00058 | 1154 | 1196 | Ldl_receptor_rep |
| Pfam | PF00058 | 1198 | 1239 | Ldl_receptor_rep |
| Pfam | PF00058 | 1241 | 1284 | Ldl_receptor_rep |
| Pfam | PF00058 | 1286 | 1326 | Ldl_receptor_rep |
| prosite | PS00010 | 501 | 512 | Asx_hydroxyl |
| prosite | PS00010 | 818 | 829 | Asx_hydroxyl |
| prosite | PS00010 | 907 | 918 | Asx_hydroxyl |
| prosite | PS00484 | 959 | 988 | Thyroglobulin_1 |
| prosite | PS00484 | 1039 | 1068 | Thyroglobulin_1 |
| prosite | PS01186 | 786 | 799 | EGF_like |
| prosite | PS01186 | 827 | 842 | EGF_like |
| prosite | PS01186 | 877 | 890 | EGF_like |
| prosite | PS01186 | 916 | 929 | EGF_like |
| prosite | PS01187 | 801 | 827 | EGF_Ca |
| prosite | PS01187 | 892 | 916 | EGF_Ca |

Following is a ENDO180 amino acid sequence (SEQ ID NO: 20).

>gi|5174485|ref|NP_006030.1| mannose receptor, C type 2
MGPGRPAPAPWPRHLLRCVLLLGCLHLGRPGAPGDAALPEPNVFLIFSHGLQGCLEAQG
GQVRVTPACNTSLPAQRWKWVSRNRLFNLGTMQCLGTGWPGTNTLTASLGMYECDRE
ALNLRWHCRTLGDQLSLLLGARTSNISKPGTLERGDQTRSGQWRIYGSEEDLCALPYHE
VYTIQGNSHGKPCTIPFKYDNQWFHGCTSTGREDGHLWCATTQDYGKDERWGFCPIKS
NDCETFWDKDQLTDSCYQFNFQSTLSWREAWASCEQQGADLLSITEIHEQTYINGLLTG
YSSTLWIGLNDLDTSGGWQWSDNSPLKYLNWESDQPDNPSEENCGVIRTESSGGWQNR
DCSIALPYVCKKKPNATAEPTPPDRWANVKVECEPSWQPFQGHCYRLQAEKRSWQESK
KACLRGGGDLVSIHSMAELEFITKQIKQEVEELWIGLNDLKLQMNFEWSDGSLVSFTH
WHPFEPNNFRDSLEDCVTIWGPEGRWNDSPCNQSLPSICKKAGQLSQGAAEEDHGCRK
GWTWHSPSCYWLGEDQVTYSEARRLCTDHGSQLVTITNRFEQAFVSSLIYNWEGEYFW
TALQDLNSTGSFFWLSGDEVMYTHWNRDQPGYSRGGCVALATGSAMGLWEVKNCTS
FRARYICRQSLGTPVTPELPGPDPTPSLTGSCPQGWASDTKLRYCYKVFSSERLQDKKS
WVQAQGACQELGAQLLSLASYEEEHFVANMLNKIFGESEPEIHEQHWFWIGLNRRDPR
GGQSWRWSDGVGFSYHNFDRSRHDDDDIRGCAVLDLASLQWVAMQCDTQLDWICKIP
RGTDVREPDDSPQGRREWLRFQEAEYKFFEHHSTWAQAQRICTWFQAELTSVHSQAEL
DFLSHNLQKFSRAQEQHWWIGLHTSESDGRFRWTDGSIINFISWAPGKPRPVGKDKKCV
YMTASREDWGDQRCLTALPYICKRSNVTKETQPPDLPTTALGGCPSDWIQFLNKCFQV
QGQEPQSRVKWSEAQFSCEQQEAQLVTITNPLEQAFITASLPNVTFDLWIGLHASQRDF
QWVEQEPLMYANWAPGEPSGPSPAPSGNKPTSCAVVLHSPSAHFTGRWDDRSCTEETH
GFICQKGTDPSLSPSPAALPPAPGTELSYLNGTFRLLQKPLRWHDALLLCESHNASLAYV
PDPYTQAFLTQAARGLRTPLWIGLAGEEGSRRYSWVSEEPLNYVGWQDGEPQQPGGCT
YVDVDGAWRTFfSCDTKLQGAVCGVSSGPPPPRRISYHGSCPQGLADSAWJPFREHCYSF
HMELLLGHKEARQRCQRAGGAVLSILDEMENVFVWEHLQSYEGQSRGAWLGMNFNP
KGGTLVWQDNTAVNYSNWGPPGLGPSMLSHNSCYWIQSNSGLWRPGACTNITMGVV
CKLPRAEQSSFSPSALPENPAALVVVLMAVLLLLALLTAALILYRRRQSIERGAFEGARY
SRSSSSPTEATEKNILVSDMEMNEQQE

The following Table shows domain regions in an ENDO180 amino acid sequence.

| | | AA | | |
|---|---|---|---|---|
| Prediction | Accession | start | AA | DOMAIN |
| method | id | site | end | DESCRIPTION |
| prints | PR00356 | 381 | 393 | AntifreezeII |
| prints | PR00356 | 393 | 410 | AntifreezeII |
| prints | PR00356 | 411 | 428 | AntifreezeII |
| prints | PR00356 | 441 | 457 | AntifreezeII |
| prints | PR00356 | 491 | 504 | AntifreezeII |
| prints | PR00013 | 184 | 193 | FN_Type_II |
| prints | PR00013 | 195 | 207 | FN_Type_II |
| prints | PR00013 | 212 | 227 | FN_Type_II |
| Pfam | PF00040 | 187 | 228 | FN_Type_II |
| Pfam | PF00059 | 255 | 361 | Lectin_C |
| Pfam | PF00059 | 399 | 506 | Lectin_C |
| Pfam | PF00059 | 538 | 646 | Lectin_C |
| Pfam | PF00059 | 693 | 810 | Lectin_C |
| Pfam | PF00059 | 842 | 952 | Lectin_C |
| Pfam | PF00059 | 993 | 1109 | Lectin_C |
| Pfam | PF00059 | 1142 | 1245 | Lectin_C |
| Pfam | PF00059 | 1284 | 1395 | Lectin_C |
| prosite | PS00023 | 187 | 228 | FN_Type_II |
| prosite | PS00213 | 1219 | 1232 | Lipocln_cytFABP |
| prosite | PS00615 | 335 | 359 | Lectin_C |
| prosite | PS00615 | 481 | 504 | Lectin_C |
| prosite | PS00615 | 927 | 950 | Lectin_C |
| pfscan | PS50231 | 54 | 121 | Ricin_B_lectin |

Following is a first CDK10 amino acid sequence (SEQ ID NO: 21).

>gi|32528263|ref|NP_003665.2| cyclin-dependent kinase 10 isoform 1; serine/threonine
protein kinase PISSLRE; CDC2-related protein kinase; cell division protein kinase 10;
cyclin-dependent kinase related protein [Homo sapiens]
MGRCRSVKEFEKLNRIGEGTYGIVYRARDTQTDEJVALKKVRMDKEKDGIPISSLREITL
LLRLRHPNIVELKEVVVGNHLESIFLVMGYCEQDLASLLENMPTPFSEAQVKCIVLQVL
RGLQYLHRNFIIHRDLKVSNLLMTDKGCVKTADFGLARAYGVPVKPMTPKVVTLWYR
APELLLGTFLTTQTTSIDMWAVGCILAELLAHRPLLPGTSEIHQIDLIVQLLGTPSENIWPGF
SKLPLVGQYSLRKQPYNNLKHKFPWLSEAGLRLLHFLFMYDPKKRATAGDCLESSYFK
EKPLPCEPELMPTFPHHRNKRAAPATSEGQSKRCKIP

Following is a third CDK10 amino acid sequence (SEQ ID NO: 23).

>gi|32528266|ref|NP_443714.2| cyclin-dependent kinase 10 isoforra 3; serine/threonine
protein kinase PISSLRE; CDC2-related protein kinase; cell division protein kinase 10;
cyclin-dependent kinase related protein [Homo sapiens]
MDKEKDGIPISSLREITLLLRLRHPNIVELKEVVVGNHLESIFLVMGYCEQDLASLLENM
PTPFSEAQVKCIVLQVLRGLQYLHRNFIIHRDLKVSNLLMTDKGCVKTGGCNLGQALSL
DGTW

Following is a FPGT amino acid sequence (SEQ ID NO: 24).

>gi|4503777|ref|NP_003829.1| fucose-1-phosphate guanyltransferase; GDP-beta-L-fucose
pyrophosphorylase [Homo sapiens]
MAAARDPPEVSLREATQRKLRRFSELRGKLVARGEFWDIVAITAADEKQELAYNQQLS
EKLKRKELPLGVQYHVFVDPAGAKIGNGGSTLCALQCLEKLYGDKWNSFTILLIHSGGY
SQRLPNASALGKIFTALPLGNPIYQMLELKLAMYIDFPLNMNPGILVTCADDIELYSIGEF
EFIRFDKPGFTALAHPSSLTIGTTHGVFVLDPFDDLKHRDLEYRSCHRFLHKPSIEKMYQ
FNAVCRPGNFCQQDFAGGDIADLKLDSDYVYTDSLFYMDHKSAKMLLAFYEKIGTLSC
EIDAYGDFLQALGPGATVEYTRNTSHVIKEESELVEMRQRIFHLLKGTSLNVVVLNNSK
FYHIGTTEEYLFYFTSDNSLKSELGLQSITFSIFPDLPECSGKTSCIIQSILDSRCSVAPGSVV
EYSRLGPDVSVGENCIISGSYILTKAALPAHSFVCSLSLKMNRCLKYATMAFGVQDNLK
KSXTKTLSDIKLLQFFGVCFLSCLDVWNLKVTEELFSGNKTCLSLWTARIFPVCSSLSDSVI
TSLKMLNAVKNKSAFSLNSYKLLSIEEMLIYKDVEDMITYREQIFLEISLKSSLM

Following is a CARK amino acid sequence (SEQ ID NO: 25).

>gi|7705748|ref|NP_057062.1| cardiac ankyrin repeat kinase [Homo sapiens]
MGNYKSRPTQTCTDEWKKKVSESYVITIERLEDDLQIKEKELTELRMFGSDEAFSKVNL
NYRTENGLSLLHLCCICGGKKSHIRTLMLKGLRPSRLTRNGFTALHLAVYKDNAELITSL
LHSGADIQQVGYGGLTALHIATLAGHLEAADVLLQHGANVNIQDAVFFTPLHJAAYYGH
EQVTRLLLKFGADVNVSGEVGDRPLHLASAKGFLNIAKLLMEEGSKADVNAQDNEDH
VPLHFCSRFGHHDIVKYLLQSDLEVQPHVVNIYGDTPLHLACYNGKFEVAKEIIQISGTE
SLTKENIFSETAFHSACTYGKSIDLVKFLLDQNVININHQGRDGHTGLHSACYHGHIRLV
QFLLDNGADMNLVACDPSRSSGEKDEQTCLMWAYEKGHDAIVTLLKHYKRPQDELPC
NEYSQPGGDGSYVSVPSPLGKIKSMTKEKADILLLRAGLPSHFHLQLSEIEFHEIIGSGSF
GKVYKGRCRNKIVAIKRYRANTYCSKSDVDMFCREVSILCQLNHPCVIQFVGACLNDPS
QFAIVTQYISGGSLFSLLHEQKRILDLQSKLIJAVDVAKGMEYLHNLTQPIIHRDLNSHNI
LLYEDGHAVVADFGESRFLQSLDEDNMTKQPGNLRWMAPEVFTQCTRYTIKADVFSY
ALCLWEILTGEIPFAHLKPAAAAADMAYHHIRPPIGYSIPKPISSLLIRGWNACPEGRPEF
SEVVMKLEECLCNIELMSPASSNSSGSLSPSSSSDCLVNRGGPGRSHVAALRSRFELEYA
LNARSYAALSQSAGQYSSQGLSLEEMKRSLQYTPIDKYGYVSDPMSSMHFHSCRNSSSF
EDSS

Following is a PCLO amino acid sequence (SEQ ID NO: 26).

MTSSGAQKKVKRTLPNPPPEEISTGTQSTFSTMGTVSRRRICRTNTMARAKILQDIDREL
DLVERESAKLRKKQAELDEEEKEIDAKLRYLEMGINRRKEALLKEREKRERAYLQGVA
EDRDYMSDSEVSSTRPTRIESQHGIERPRTAPQTEFSQFIPPQTQTESQLVPPTSPYTQY
QYSSPALPTQAPTSYTQQSHFEQQTLYHQQVSPYQTQPTFQAVATMSFTPQVQPTPTPQP
SYQLPSQMMVIQQKPRQTTLYLEPKITSNYEVIRNQPLMIAPVSTDNTFAVSHLGSKYNS
LDLRIGLEERSSMASSPISSISADSFYADIDHHTPRNYVLIDDIGEITKGTAALSTAFSL
HEKDLSKTDRLLRTTETRRSQEVTDFLAPLQSSSRLHSYVKAEEDPMEDPYELKLLKHQI
KQEFRRGTESLDHLAGLSHYYHADTSYRHFPKSEKYSISRLTLEKQAAKQLPAAILYQKQ
SKHKKSLIDPKMSKFSPIQESRDLEPDYSSYMTSSTSSIGGISSRARLLQDDITFGLRKN
ITDQQKFMGSSLGTGLGTLGNTIRSALQDEADKPYSSGSRSRPSSRPSSVYGLDLSIKRD
SSSSSLRLKAQEAEALDVSFSHASSSARTKPTSLPISQSRGRIPIVAQNSEEESPLSPVG
QPMGMARAAAGPLPPISADTRDQFGSSHSLPEVQQHMREESRTRGYDRDIAFIMDDFQHA
MSDSEAYHLRREETDWFDKPRESRLENGHGLDRKLPERLVHSRPLSQHQEQIIQMNGKTM
HYIFPHARIKITRDSKDHTVSGNGLGIRLVGGKEIPGHSGEIGAYIAKILPGGSAEQTGK
IMEGMQVLEWNGIPLTSKTYEEVQSIISQQSGEAEICVRLDLNMLSDSENSQHLELHEPP
KAVDKAKSPGVDPKQLAAELQKVSLQQSPLVLSSVVEKGSHVHSGPTSAGSSSVPSPGQP
GSPSVSKKKHGSSKPTDGTKVVSHPITGEIQLQINYDLGNLIIHILQARNLVPRDNNGYS
DPFVKVYLLPGRGQVMVVQNASAEYKRRTKHVQKSLNPEWNQTVJYKSISMEQLKKKTLE
VTVWDYDRFSSNDFLGEVLIDLSSTSHLDNTPRWYPLKEQTESIDHGKSHSSQSSQQSPK
PSVIKSRSHGWPDPSKDMQVPTIEKSHSSPGSSKSSSEGHLRSHGPSRSQSKTSVTQTHL
EDAGAAIAAAEAAVQQLRIQPSKRRK

Following is a REPS2 amino acid sequence (SEQ ID NO: 27)

>gi|4758944|ref|NP_004717.1| RALBP1 associated Eps domain containing 2; partner of
Ral-binding protein 1 [Homo sapiens]
MMSKNDGELRFGNPAELHGTKVQIPYLTTEKNSFKRMDDEDKQQETQSPTMSPLASPPS
SPPHYQRVPLSHGYSKLRSSAEQMHPAPYEARQPLVQPEGSSSGGPGTKPLRHQASLIRS
FSVERELQDNSSYPDEPWRITEEQREYYVNQFRSLQPDPSSFISGSVAKNFFTKSKLSIPE
LSYIWELSDADCDGALTLPEFCAAFHLIVARKNGYPLPEGLPPTLQPEYLQAAFPKPKW
DCQLFDSYSESLPANQQPRDLNRMEKTSVKDMADLPVPNQDVTSDDKQALKSTINEAL
PKDVSEDPATPKDSNSLKARPRSRSYSSTSIEEAMKRGEDPPTPPPRPQKTHSRASSLDLN
KVFQPSVPATKSGLLPPPPALPPRPCPSQSEQVSEAELLPQLSRAPSQAAESSPAKKDVLY
SQPPSKPIRRKFRPENQATENQEPSTAASGPASAATMKPHPTVQKQSSKQKKAIQTAIRK
NKEANAVLARLNSELQQQLKEVHQERIALENQLEQLRPVTVL

Modifications may be made to the foregoing without departing from the basic aspects of the invention. Although the invention has been described in substantial detail with reference to one or more specific embodiments, those of skill in the art will recognize that changes may be made to the embodiments specifically disclosed in this application, yet these modifications and improvements are within the scope and spirit of the invention, as set forth in the claims which follow. Also, citation of the above publications or documents is not intended as an admission that any of the foregoing is pertinent prior art, nor does it constitute any admission as to the contents or date of these publications or documents. U.S. patents and other publications and documents referenced are incorporated herein by reference.

1. A method for identifying a subject at risk of melanoma, which comprises detecting the presence or absence of a polymorphic variation associated with melanoma at one or more positions selected from the group consisting of rs6481845, rs10643659, rs7475391, rs7475394, rs7475396, rs9418057, rs5784335, rs2429390, rs7209331, rs4968706, rs8072984, rs9896444, rs4968617, rs7350907, rs8072519, rs6467914, rs3064869, rs10555175, rs6956848, position 63624 in SEQ ID NO: 6, position 65039 in SEQ ID NO: 6, rs489941, rs496008, rs4650251, rs531412, rs3765654, rs6660350, position 206 in SEQ ID NO: 5, rs5924619, rs5924564, rs4828523, rs5924569, rs2052763, rs1549529, rs5924573, rs4240072, rs5924580, rs4828544, rs5969758, rs1965051, rs1426800, rs1365529, rs5924545, rs5969761, rs6629202, rs5924584, rs1365530, rs6527769, rs2382813, rs2033120, rs2162394, rs1346246, rs1365527, rs4828524, rs5924588, rs5969764, rs5924589, rs5924590, rs5924593, rs4828547, rs5969768, rs4828526, rs5924595, rs5924549, rs5969769, rs4007744, rs2891073, rs5924599, rs1549531, rs5924601, rs5901609, rs5969739, rs5924602, rs1426798, rs4828550, rs5924603, rs1807947, rs2382814, rs5924606 and position 25216 in SEQ ID NO: 7;
whereby the presence of the one or more polymorphic variations is indicative of the subject being at risk of melanoma.

2. The method of claim 1, which further comprises obtaining the nucleic acid sample from the subject.

3. The method of claim 1, wherein a polymorphic variation is detected at one or more positions selected from the group consisting of rs6481845, rs10643659, rs7475391, rs7475394, rs7475396, rs9418057 and rs5784335.

4. The method of claim 1, wherein a polymorphic variation is detected at one or more positions selected from the group consisting of rs2429390, rs7209331, rs4968706, rs8072984, rs9896444, rs4968617, rs7350907 and rs8072519.

5. The method of claim 1, wherein a polymorphic variation is detected at one or more positions selected from the group consisting of rs6467914, rs3064869, rs10555175, rs6956848, position 63624 in SEQ ID NO: 6 and position 65039 in SEQ ID NO: 6.

6. The method of claim 1, wherein a polymorphic variation is detected at one or more positions selected from the group consisting of rs489941, rs496008, rs4650251, rs531412, rs3765654, rs6660350 and position 206 in SEQ ID NO: 5.

7. The method of claim 1, wherein a polymorphic variation is detected at one or more positions selected from the group consisting of rs5924619, rs5924564, rs4828523, rs5924569, rs2052763, rs1549529, rs5924573, rs4240072, rs5924580, rs4828544, rs5969758, rs1965051, rs1426800, rs1365529, rs5924545, rs5969761, rs6629202, rs5924584, rs1365530, rs6527769, rs2382813, rs2033120, rs2162394, rs1346246, rs1365527, rs4828524, rs5924588, rs5969764, rs5924589, rs5924590, rs5924593, rs4828547, rs5969768, rs4828526, rs5924595, rs5924549, rs5969769, rs4007744, rs2891073, rs5924599, rs1549531, rs5924601, rs5901609, rs5969739, rs5924602, rs1426798, rs4828550, rs5924603, rs1807947, rs2382814, rs5924606 and position 25216 in SEQ ID NO: 7.

8. The method of claim 1, wherein one or more polymorphic variations are detected at one or more positions in linkage disequilibrium with a polymorphic variation at one or more of the positions in claim 1.

9. The method of claim 1, wherein detecting the presence or absence of the one or more polymorphic variations comprises:
hybridizing an oligonucleotide to the nucleic acid sample, wherein the oligonucleotide is complementary to a nucleotide sequence in the nucleic acid and hybridizes to a region adjacent to the polymorphic variation;
extending the oligonucleotide in the presence of one or more nucleotides, yielding extension products; and
detecting the presence or absence of a polymorphic variation in the extension products.

10. The method of claim 1, wherein the subject is a human.

11. A method for identifying a polymorphic variation associated with melanoma proximal to an incident polymorphic variation associated with melanoma, which comprises:
identifying a polymorphic variation proximal to the incident polymorphic variation associated with melanoma, wherein the incident polymorphic variation is at a position selected from the group consisting of rs6481845, rs10643659, rs7475391, rs7475394, rs7475396, rs9418057, rs5784335, rs2429390, rs7209331, rs4968706, rs8072984, rs9896444, rs4968617, rs7350907, rs8072519, rs6467914, rs3064869, rs10555175, rs6956848, position 63624 in SEQ ID NO: 6, position 65039 in SEQ ID NO: 6, rs489941, rs496008, rs4650251, rs531412, rs3765654, rs6660350, position 206 in SEQ ID NO: 5, rs5924619, rs5924564, rs4828523, rs5924569, rs2052763, rs1549529, rs5924573, rs4240072, rs5924580, rs4828544, rs5969758, rs1965051, rs1426800, rs1365529, rs5924545, rs5969761, rs6629202, rs5924584, rs1365530, rs6527769, rs2382813, rs2033120, rs2162394, rs1346246, rs1365527, rs4828524, rs5924588, rs5969764, rs5924589, rs5924590, rs5924593, rs4828547, rs5969768, rs4828526, rs5924595, rs5924549, rs5969769, rs4007744, rs2891073, rs5924599, rs1549531, rs5924601, rs5901609, rs5969739, rs5924602, rs1426798, rs4828550, rs5924603, rs1807947, rs2382814, rs5924606 and position 25216 in SEQ ID NO: 7; and
determining the presence or absence of an association of the proximal polymorphic variant with melanoma.

12. The method of claim 11, wherein the proximal polymorphic variation is within a region between about 5 kb 5′ of the incident polymorphic variation and about 5 kb 3′ of the incident polymorphic variation.

13. The method of claim 11, which further comprises determining whether the proximal polymorphic variation is at a position in linkage disequilibrium with the incident polymorphic variation.

14. The method of claim 11, which further comprises identifying a second polymorphic variation proximal to the identified proximal polymorphic variation associated with melanoma and determining if the second proximal polymorphic variation is associated with melanoma.

15. The method of claim 14, wherein the second proximal polymorphic variant is within a region between about 5 kb 5′ of the incident polymorphic variation and about 5 kb 3′ of the proximal polymorphic variation associated with melanoma.

16. An isolated nucleic acid which comprises a portion of or all of a nucleotide sequence in SEQ ID NO: 3, 4, 5, 6 and/or 7 and comprises one or more polymorphic variations selected from the group consisting of an adenine at position 16727 in SEQ ID NO: 3, a guanine at position 36589 in SEQ ID NO: 4, a thymine at position 44544 in SEQ ID NO: 4, a guanine at position 48304 in SEQ ID NO: 4, a guanine at position 53843 in SEQ ID NO: 6, a guanine at position 63624 in SEQ ID NO: 6, an adenine-thymine deletion at position 65039 in SEQ ID NO: 6, a guanine at position 206 in SEQ ID NO: 5, an adenine at position 9261 in SEQ ID NO: 5, a thymine at position 37592 in SEQ ID NO: 5, a guanine at position 10537 in SEQ ID NO: 7, a cytosine at position 25216 in SEQ ID NO: 7 and a thymine at position 119368 in SEQ ID NO: 7.

17. The isolated nucleic acid of claim 16, which comprises an adenine at position 16727 in SEQ ID NO: 3.

18. The isolated nucleic acid of claim 16, which comprises a polymorphic variation selected from the group consisting of a guanine at position 36589 in SEQ ID NO: 4, a thymine at position 44544 in SEQ ID NO: 4, a guanine at position 48304 in SEQ ID NO: 4.

19. The isolated nucleic acid of claim 16, which comprises a polymorphic variation selected from the group consisting of a guanine at position 53843 in SEQ ID NO: 6, a guanine at position 63624 in SEQ ID NO: 6, an adenine-thymine deletion at position 65039 in SEQ ID NO: 6.

20. The isolated nucleic acid of claim 16, which comprises a polymorphic variation selected from the group consisting of a guanine at position 206 in SEQ ID NO: 5, an adenine at position 9261 in SEQ ID NO: 5, a thymine at position 37592 in SEQ ID NO: 5.

21. The isolated nucleic acid of claim 16, which comprises a polymorphic variation selected from the group consisting of a guanine at position 10537 in SEQ ID NO: 7, a cytosine at position 25216 in SEQ ID NO: 7 and a thymine at position 119368 in SEQ ID NO: 7.

22. An oligonucleotide comprising a nucleotide sequence complementary to a portion of the nucleotide sequence of claim 16, wherein the 3′ end of the oligonucleotide is adjacent to a polymorphic variation.

23. A microarray comprising an isolated nucleic acid of claim 16 linked to a solid support.

24. An isolated polypeptide encoded by the isolated nucleic acid sequence of claim 16.

25. A method of genotyping a nucleic acid at a position selected from the group consisting of position 16727 in SEQ ID NO: 3, position 36589 in SEQ ID NO: 4, position 44544 in SEQ ID NO: 4, position 48304 in SEQ ID NO: 4, position 53843 in SEQ ID NO: 6, position 63624 in SEQ ID NO: 6, position 65039 in SEQ ID NO: 6, position 206 in SEQ ID NO: 5, position 9261 in SEQ ID NO: 5, position 37592 in SEQ ID NO: 5, position 10537 in SEQ ID NO: 7, position 25216 in SEQ ID NO: 7 and position 119368 in SEQ ID NO: 7.

26. The method of claim 25, which comprises genotyping the nucleic acid at position 16727 in SEQ ID NO: 3.

27. The method of claim 25, which comprises genotyping the nucleic acid at a position selected from the group consisting of position 36589 in SEQ ID NO: 4, position 44544 in SEQ ID NO: 4 and position 48304 in SEQ ID NO: 4.

28. The method of claim 25, which comprises genotyping the nucleic acid at a position selected from the group consisting of position 53843 in SEQ ID NO: 6, position 63624 in SEQ ID NO: 6 and position 65039 in SEQ ID NO: 6.

29. The method of claim 25, which comprises genotyping the nucleic acid at a position selected from the group consisting of position 206 in SEQ ID NO: 5, position 9261 in SEQ ID NO: 5 and position 37592 in SEQ ID NO: 5.

30. The method of claim 25, which comprises genotyping the nucleic acid at a position selected from the group consisting of position 10537 in SEQ ID NO: 7, position 25216 in SEQ ID NO: 7 and position 119368 in SEQ ID NO: 7.

31. A method for identifying a candidate molecule that modulates cell proliferation, which comprises:
(a) introducing a test molecule to a system which comprises one or more cells and a a nucleic acid comprising one or more polymorphic variations at one or more positions selected from the group consisting of rs6481845, rs10643659, rs7475391, rs7475394, rs7475396, rs9418057, rs5784335, rs2429390, rs7209331, rs4968706, rs8072984, rs9896444, rs4968617, rs7350907, rs8072519, rs6467914, rs3064869, rs10555175, rs6956848, position 63624 in SEQ ID NO: 6, position 65039 in SEQ ID NO: 6, rs489941, rs496008, rs4650251, rs531412, rs3765654, rs6660350, position 206 in SEQ ID NO: 5, rs5924619, rs5924564, rs4828523, rs5924569, rs2052763, rs1549529, rs5924573, rs4240072, rs5924580, rs4828544, rs5969758, rs1965051, rs1426800, rs1365529, rs5924545, rs5969761, rs6629202, rs5924584, rs1365530, rs6527769, rs2382813, rs2033120, rs2162394, rs1346246, rs1365527, rs4828524, rs5924588, rs5969764, rs5924589, rs5924590, rs5924593, rs4828547, rs5969768, rs4828526, rs5924595, rs5924549, rs5969769, rs4007744, rs2891073, rs5924599, rs1549531, rs5924601, rs5901609, rs5969739, rs5924602, rs1426798, rs4828550, rs5924603, rs1807947, rs2382814, rs5924606 and position 25216 in SEQ ID NO: 7; and
(b) determining whether cell proliferation is increased or decreased compared to a system not containing the test molecule,
whereby a increased or decreased cell proliferation identifies the test molecule as a candidate molecule that modulates cell proliferation.

32. The method of claim 31, wherein the system is an animal.

33. The method of claim 31, wherein the system is one or more cells.

34. A method for treating melanoma in a subject, which comprises administering a candidate molecule identified by the method of claim 31 to a subject in need thereof, whereby the candidate molecule treats melanoma in the subject.

35. A method for treating melanoma in a subject or preventing melanoma in a subject, which comprises detecting the presence or absence of one or more polymorphic variations at one or more positions selected from the group consisting of rs6481845, rs10643659, rs7475391, rs7475394, rs7475396, rs9418057, rs5784335, rs2429390, rs7209331, rs4968706, rs8072984, rs9896444, rs4968617, rs7350907, rs8072519, rs6467914, rs3064869, rs10555175, rs6956848, position 63624 in SEQ ID NO: 6, position 65039 in SEQ ID NO: 6, rs489941, rs496008, rs4650251, rs531412, rs3765654, rs6660350, position 206 in SEQ ID NO: 5, rs5924619, rs5924564, rs4828523, rs5924569, rs2052763, rs1549529, rs5924573, rs4240072, rs5924580, rs4828544, rs5969758, rs1965051, rs1426800, rs1365529, rs5924545, rs5969761, rs6629202, rs5924584, rs1365530, rs6527769, rs2382813, rs2033120, rs2162394, rs1346246, rs1365527, rs4828524, rs5924588, rs5969764, rs5924589, rs5924590, rs5924593, rs4828547, rs5969768, rs4828526, rs5924595, rs5924549, rs5969769, rs4007744, rs2891073, rs5924599, rs1549531, rs5924601, rs5901609, rs5969739, rs5924602, rs1426798, rs4828550, rs5924603, rs1807947, rs2382814, rs5924606 and position 25216 in SEQ ID NO: 7; and
administering a melanoma treatment or administering a melanoma preventative to a subject in need thereof based upon the presence or absence of the one or more polymorphic variations in the nucleic acid sample.

36. The method of claim 35, wherein the one or more polymorphic variations is at one or more positions selected from the group consisting of rs6481845, rs10643659, rs7475391, rs7475394, rs7475396, rs9418057 and rs5784335.

37. The method of claim 35, wherein the one or more polymorphic variations is at one or more positions selected from the group consisting of rs2429390, rs7209331, rs4968706, rs8072984, rs9896444, rs4968617, rs7350907 and rs8072519.

38. The method of claim 35, wherein a polymorphic variation is at position rs8404.

39. The method of claim 35, wherein the one or more polymorphic variations is at one or more positions selected from the group consisting of rs6467914, rs3064869, rs10555175, rs6956848, position 63624 in SEQ ID NO: 6 and position 65039 in SEQ ID NO: 6.

40. The method of claim 35, wherein the one or more polymorphic variations is at one or more positions selected from the group consisting of rs489941, rs496008, rs4650251, rs531412, rs3765654, rs6660350 and position 206 in SEQ ID NO: 5.

41. The method of claim 35, wherein the one or more polymorphic variations is at one or more positions selected from the group consisting of rs5924619, rs5924564, rs4828523, rs5924569, rs2052763, rs1549529, rs5924573, rs4240072, rs5924580, rs4828544, rs5969758, rs1965051, rs1426800, rs1365529, rs5924545, rs5969761, rs6629202, rs5924584, rs1365530, rs6527769, rs2382813, rs2033120, rs2162394, rs1346246, rs1365527, rs4828524, rs5924588, rs5969764, rs5924589, rs5924590, rs5924593, rs4828547, rs5969768, rs4828526, rs5924595, rs5924549, rs5969769, rs4007744, rs2891073, rs5924599, rs1549531, rs5924601, rs5901609, rs5969739, rs5924602, rs1426798, rs4828550, rs5924603, rs1807947, rs2382814, rs5924606 and position 25216 in SEQ ID NO: 7.

42. The method of claim 35, which further comprises extracting and analyzing a tissue biopsy sample from the subject.

43. The method of claim 35, wherein the treatment is one or more selected from the group consisting of administering cisplatin, administering carmustine, administering vinblastine, administering vincristine, administering bleomycin, administering a combination of the foregoing, and surgery.

44. The method of claim 35, wherein the preventative reduces ultraviolet (UV) light exposure to the subject.
